(19) United States
(12) Patent Application Publication
(10) Pub. No.: US 2023/0200219 A1
(43) Pub. Date: Jun. 22, 2023

(54) A compound, an organic electroluminescent device and a display device

(71) Applicant: BEIJING ETERNAL MATERIAL TECHNOLOGY CO., LTD, Beijing (CN)

(72) Inventors: Jinhua HUANG, Beijing (CN); Lichang ZENG, Beijing (CN); Long SUN, Beijing (CN); Song LIU, Beijing (CN)

(21) Appl. No.: 17/924,692

(22) PCT Filed: Mar. 19, 2021
(86) PCT No.: PCT/CN2021/081915

§ 371 Date: Nov. 11, 2022

(30) Foreign Application Priority Data
May 11, 2020 (CN) 202010394523.4
Oct. 29, 2020 (CN) 202011181935.6
Oct. 29, 2020 (CN) 202011186015.3

(51) Int. Cl. H10K 85/60; C07C 211/61; C07D 333/76; C07D 307/91

(52) U.S. Cl. CPC H10K 85/633; C07C 211/61; C07D 333/76; H10K 85/636; C07D 307/91; H10K 85/626; C07C 2602/10; C07C 2603/18; C07C 2603/40; H10K 85/6576; H10K 85/6574; H10K 50/181

(57) ABSTRACT

The disclosure provides a compound, an organic electroluminescent device and a display device. Wherein, the compound has a structure as shown in Formula I. The introduction of a substituent Ar² at the 1-position of naphthalene ring can not only adjust the adjacent steric hindrance size, but also effectively regulate the distortion degree of a molecule so as to reduce the crystallinity of the molecule. Secondly, an arylamino group is introduced at the 2-position, and the introduction of a trisubstituted structure on the arylamino group can effectively regulate the stereostructure of the molecule, improve the packing density of the molecule, so that the materials designed can meet the requirements of devices for materials. The compound of the disclosure can improve the luminous efficiency, reduce the starting voltage and prolong the service life of the device when it is applied to the organic electroluminescent device.

### CROSS-REFERENCE TO RELATED APPLICATIONS

The present application is a National Stage of International Patent Application No: PCT/CN2021/081915 filed on Mar. 19, 2021.

### TECHNICAL FIELD

The disclosure relates to the technical field of organic electroluminescence, in particular to a compound, an organic electroluminescent device and a display device.

### BACKGROUND

In recent years, optoelectronic devices based on organic materials have become more and more popular. The inherent flexibility of organic materials makes them very suitable for manufacturing on flexible substrates. They can be designed according to needs to produce beautiful and cool optoelectronic products, obtaining incomparable advantages over inorganic materials. Examples of such organic optoelectronic devices include organic light emitting diodes (OLEDs), organic field-effect transistors, organic photovoltaic cells, and organic sensors, etc. In particular, OLEDs have developed rapidly and have achieved commercial success in the field of information display. OLEDs can provide red, green and blue colors with high saturation, and the full-color display devices made of OLEDs do not need additional backlight, and have the advantages of dazzling colors, light and thin as well as softness, etc.

The core of OLED device is a thin film structure containing a variety of organic functional materials. Common functional organic materials include: a hole injection material, a hole transporting material, a hole blocking material, an electron injection material, an electron transporting materials, an electron blocking material, a light emitting host material and a light emitting guest material (an dye), etc. When electrified, electrons and holes are injected and transmitted to the light emitting region, respectively and recombined therein, thus generating excitons and emitting light.

A variety of organic materials have been developed, combined with various peculiar device structures, to enhance the carrier mobility, regulate the carrier balance, break through the electroluminescence efficiency, and delay the device attenuation. For the reason of quantum mechanics, the common fluorophors mainly use the singlet excitons generated when electrons and holes combine to emit light, which are still widely used in various OLED products. Some metal complexes, such as iridium complexes, can use both triplet excitons and singlet excitons to emit light at the same time, they are called phosphors, and their energy conversion efficiency can be improved by up to four times compared with traditional fluorophors. Thermally activated delayed fluorescence (TADF) technology can promote the transition from a triplet exciton to a singlet exciton, and the triplet exciton can still be effectively used to achieve high luminous efficiency without the use of metal complexes. Thermally activated sensitized fluorescence (TASF) technology uses materials with TADF properties to sensitize the luminophors by means of energy transfer, which can also achieve high luminous efficiency.

As OLED products enter the market gradually, people have higher and higher requirements for the performance of such products. The current used OLED materials and device structures cannot completely solve the problems such as efficiency, lifespan, and cost, etc.; of the OLED product.

Therefore, there is an urgent need in the art to develop an organic electroluminescent material that can improve the luminous efficiency, reduce the driving voltage, and extend the service life, of the device, so as to develop more types and higher performance of OLED devices.

### SUMMARY

The main object of the disclosure is to provide a compound, an organic electroluminescent device and a display device to provide a new organic electroluminescent material so as to improve the luminous efficiency of OLED devices.

The first object of the disclosure is to provide a compound, which can be applied to the organic electroluminescent device so as to improve the luminous efficiency, reduce the driving voltage and extend the service life of the device.

To achieve this object, the disclosure provides a compound, which has a structure as shown in Formula I;

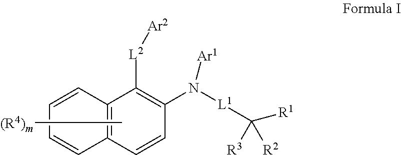

In Formula I, the Ar¹ and Ar² are independently selected from the group consisting of a substituted or unsubstituted C6-C30 (e.g., C10, C12, C14, C16, C18, C20, C26, C28, etc.) aryl or a substituted or unsubstituted C3-C30 (e.g., C4, C6, C8, C12, C15, C18, C20, C23, C25, C28, etc.) heteroaryl;

the substituted or unsubstituted C6-C30 aryl includes C6-C30 monocyclic aryl and C10-C30 fused ring aryl; and the substituted or unsubstituted C3-C30 heteroaryl includes C3-C30 monocyclic heteroaryl and C6-C30 fused ring heteroaryl;

In Formula I, the L is selected from the group consisting of a substituted or unsubstituted C6-C30 (e.g., C10, C12, C14, C16, C18, C20, C26, C28, etc.) arylene or a substituted or unsubstituted C3-C30 (e.g., C4, C6, C8, C12, C15, C18, C20, C23, C25, C28, etc.) heteroarylene;

In Formula I, the L² is selected from the group consisting of one of a single bond, a substituted or unsubstituted C6-C30 (e.g., C10, C12, C14, C16, C18, C20, C26, C28, etc.) arylene or a substituted or unsubstituted C3-C30 (e.g., C4, C6, C8, C12, C15, C18, C20, C23, C25, C28, etc.) heteroarylene;

In Formula I, the R¹, R² and R³ are independently selected from the group consisting of any one of a substituted or unsubstituted C1-C20 (e.g., C2, C3, C4, C5, C6, C7, C8, C9, C10, C11, C12, C13, C14, C15, C16, C17, C18, C19, etc.) chain alkyl, a substituted or unsubstituted C3-C20 (e.g., C4, C5, C6, C7, C8, C9, C10, C11, C12, C13, C14, C15, C16, C17, C18, C19, etc.) cycloalkyl, a substituted or unsubstituted C1-C20 (e.g., C2, C3, C4, C5, C6, C7, C8, C9, C10, C11, C12, C13, C14, C15, C16, C17, C18, C19, etc.) chain alkoxy, a substituted or unsubstituted C3-C20 (e.g., C4, C5, C6, C7, C8, C9, C10, C11, C12, C13, C14, C15, C16, C17, C18, C19, etc.) cycloalkoxy, a substituted or unsubstituted C2-C10 (e.g., C2, C3, C4, C5, C6, C7, C8, C9, etc.) alkenyl, a substituted or unsubstituted C2-C10 (e.g., C2, C3, C4, C5, C6, C7, C8, C9, etc.) alkynyl, halogen, cyano, nitro, hydroxyl, C1-C20 (e.g., C2, C3, C4, C5, C6, C7, C8, C9, C10, C11, C12, C13, C14, C15, C16, C17, C18, C19, etc.)silyl, amino, a substituted or unsubstituted C6-C30 (e.g., C10, C12, C14, C16, C18, C20, C26, C28, etc.) arylamino, a substituted or unsubstituted C3-C30 (e.g., C4, C6, C8, C12, C15, C18, C20, C23, C25, C28, etc.) heteroarylamino, a substituted or unsubstituted C6-C30 (e.g., C10, C12, C14, C16, C18, C20, C26, C28, etc.) aryl, a substituted or unsubstituted C3-C30 (e.g., C4, C6, C8, C12, C15, C18, C20, C23, C25, C28, etc.) heteroaryl; and the R¹, R² and R³ are connected to form a ring or not connected to form a ring;

In Formula I, the m is an integer from 0 to 6, for example, 1, 2, 3, 4, 5, etc.; when m is greater than or equal to 2, R⁴ is the same or different;

In Formula I, the R⁴ is independently selected from the group consisting of one of a substituted or unsubstituted C1-C20 (e.g., C2, C3, C4, C5, C6, C7, C8, C9, C10, C11, C12, C13, C14, C15, C16, C17, C18, C19, etc.) chain alkyl, a substituted or unsubstituted C3-C20 (e.g., C4, C5, C6, C7, C8, C9, C10, C11, C12, C13, C14, C15, C16, C17, C18, C19, etc.) cycloalkyl, a substituted or unsubstituted C1-C20 (e.g., C2, C3, C4, C5, C6, C7, C8, C9, C10, C11, C12, C13, C14, C15, C16, C17, C18, C19, etc.) chain alkoxy, a substituted or unsubstituted C3-C20 (e.g., C4, C5, C6, C7, C8, C9, C10, C11, C12, C13, C14, C15, C16, C17, C18, C19, etc.) cycloalkoxy, a substituted or unsubstituted C2-C10 (e.g., C2, C3, C4, C5, C6, C7, C8, C9, etc.) alkenyl, a substituted or unsubstituted C2-C10 (e.g., C2, C3, C4, C5, C6, C7, C8, C9, etc.) alkynyl, halogen, cyano, nitro, hydroxyl, a substituted or unsubstituted C1-C20 (e.g., C2, C3, C4, C5, C6, C7, C8, C9, C10, C11, C12, C13, C14, C15, C16, C17, C18, C19, etc.)silyl, amino, a substituted or unsubstituted C6-C30 (e.g., C10, C12, C14, C16, C18, C20, C26, C28, etc.) arylamino, a substituted or unsubstituted C3-C30 (e.g., C4, C6, C8, C12, C15, C18, C20, C23, C25, C28, etc.) heteroarylamino, a substituted or unsubstituted C6-C30 (e.g., C10, C12, C14, C16, C18, C20, C26, C28, etc.) aryl, a substituted or unsubstituted C3-C30 (e.g., C4, C6, C8, C12, C15, C18, C20, C23, C25, C28, etc.) heteroaryl;

In Ar¹, Ar², L¹, L², R¹, R², R³ and R⁴, the substituent is selected from the group consisting of one or a combination of at least two of halogen, C1-C10 (e.g., C2, C3, C4, C5, C6, C7, C8, C9, etc.) chain alkyl, C3-C10 (e.g., C4, C5, C6, C7, C8, C9, etc.) cycloalkyl, C1-C10 (e.g., C2, C3, C4, C5, etc.) alkoxy, C1-C10 (e.g., C2, C3, C4, C5, etc.) thioalkoxy, C6-C30 (e.g., C10, C12, C14, C16, C18, C20, C26, C28, etc.) arylamino, C3-C30 (e.g., C4, C6, C8, C12, C15, C18, C20, C23, C25, C28, etc.) heteroarylamino, C6-C30 (e.g., C10, C12, C14, C16, C18, C20, C26, C28, etc.) monocyclic aryl, C10-C30 (e.g., C10, C12, C14, C16, C18, C20, C26, C28, etc.) fused ring aryl, C3-C30 (e.g., C4, C6, C8, C12, C15, C18, C20, C23, C25, C28, etc.) monocyclic heteroaryl, and C6-C30 (e.g., C10, C12, C14, C16, C18, C20, C26, C28, etc.) fused ring heteroaryl.

The aforementioned “substituted or unsubstituted” groups can be substituted with one substituent or multiple substituents. When the substituents are multiple, they can be selected from different substituents and have the same meaning when the disclosure involves the same expression, and the selection range of the substituents is as shown above and will not be repeated herein.

In the disclosure, the expression of chemical elements includes the concept of isotopes with the same chemical properties. For example, hydrogen (H) includes ¹H (protium or H), ²H (deuterium or D), etc; and carbon (C) includes ¹²C and ¹³C, etc.

In the disclosure, the heteroatom of a heteroaryl group usually refers to one selected from N, O, and S.

In the disclosure, the expression of the ring structure crossed by “—” indicates that the connection site is at any position on the ring structure where bonds can be formed.

The above-mentioned C1-C20 chain alkyl is preferably C1-C10 chain alkyl, more preferably C1-C6 chain alkyl, for example, methyl, ethyl, n-propyl, iso-propyl, n-butyl, iso-butyl, tert-butyl, n-hexyl, n-octyl, n-pentyl, n-heptyl, n-nonyl, n-decyl, etc., can be listed.

The above-mentioned C3-C20 cycloalkyl is preferably cyclopropyl, cyclobutyl, cyclopentyl, and cyclohexyl.

The above-mentioned substituted or unsubstituted C6-C30 aryl, preferably C6-C20 aryl, and preferably the aryl is a group from the group consisting of phenyl, biphenyl, terphenyl, naphthyl, anthracyl, phenanthryl, indenyl, fluorenyland derivatives thereof, fluoranthenyl, triphenylene, pyrenyl, perylene, chrysenyl and tetracenyl. The biphenyl is selected from the group consisting of 2-biphenyl, 3-biphenyl and 4-biphenyl; the terphenyl includes para-terphenyl-4-yl, para-terphenyl-3-yl, para-terphenyl-2-yl, meta-terphenyl-4-yl, meta-terphenyl-3-yl and meta-terphenyl-2-yl; the naphthyl includes 1-naphthyl or 2-naphthyl; the anthracyl is selected from the group consisting of 1-anthracyl, 2-anthracyl and 9-anthracyl; the fluorenyl is selected from the group consisting of 1-fluorenyl, 2-fluorenyl, 3-fluorenyl, 4-fluorenyl and 9-fluorenyl; the fluorenyl derivative is selected from the group consisting of 9,9′-dimethylfluorene, 9,9′-spirobifluorene and benzofluorene; the pyrenyl is selected from the group consisting of 1-pyrenyl, 2-pyrenyl and 4-pyrenyl; and the tetracenyl is selected from the group consisting of 1-tetracenyl, 2-tetracenyl and 9-tetracenyl.

The above-mentioned substituted or unsubstituted C3-C30 heteroaryl, preferably C4-C20 heteroaryl, preferably the heteroaryl is furanyl, thienyl, pyrrolyl, benzofuranyl, benzothienyl, isobenzofuranyl, indolyl, dibenzofuranyl, dibenzothiophenyl, carbazolyl and derivatives thereof, wherein, the carbazolyl derivative is preferably 9-phenylcarbazole, 9-naphthylcarbazolebenzocarbazole, dibenzocarbazole, or indolocarbazole.

In the disclosure, the labels of substitution sites on the naphthalene ring are as follows:

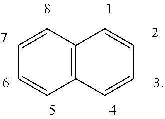

In the structure of compounds of the disclosure, the introduction of a substituent Ar² at the 1-position of naphthalene ring can not only adjust the 1-position steric hindrance size, but also effectively regulate the distortion degree of a molecule so as to reduce the crystallinity of the molecule. Secondly, the 2-position is substituted with an arylamino group, and the introduction of a trisubstituted structure

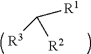

on the arylamino group can effectively regulate the stereostructure of the molecule, improve the packing density of the molecule, so that the materials designed can meet the requirements of devices for materials.

In the disclosure, the naphthalene ring parent core structure substituted at the 1- and 2-position at the same time and coordinated with substituents such as Ar¹, Ar², R¹-R⁴, etc., can achieve the best effect, so that the material can improve the luminous efficiency, reduce the starting voltage and prolong the service life of the device when it is applied to the organic electroluminescent device, especially when it is used as an electron blocking layer.

In addition, the preparation process of the compound of the disclosure is simple and practicable, and the raw materials are ready available, which is suitable for large scale production.

Preferably, the L² is selected from the group consisting of one of a single bond, a substituted or unsubstituted C6-C20 arylene or a substituted or unsubstituted C3-C20 heteroarylene, preferably a single bond or phenylene.

Preferably, the Ar² is selected from the group consisting of a substituted or unsubstituted C6-C20 aryl or a substituted or unsubstituted C3-C20 heteroaryl.

Preferably, the A² is selected from the group consisting of any of the following substituted or unsubstituted groups: phenyl, biphenyl, terphenyl, naphthyl, phenanthryl, anthracyl, dibenzofuranyl, dibenzothiophenyl, carbazolyl, 9,9dimethylfluorenyl, 9,9diphenylfluorenyl, spirofluorenyl, triphenylene, fluoranthenyl, benzo9,9dimethylfluorenyl, and benzospirofluorenyl.

Preferably, the Ar² is selected from the group consisting of any of the following substituted or unsubstituted groups:

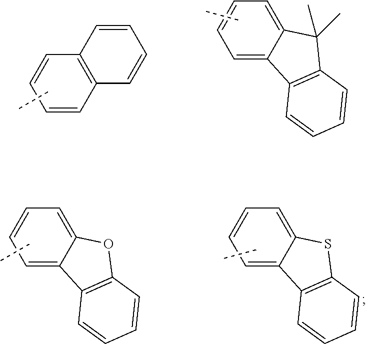

wherein, the dotted line represents a connecting bond of the groups.

Preferably, the L² is a single bond, and the Ar² is selected from the group consisting of a substituted or unsubstituted C10-C30 fused ring aryl or a substituted or unsubstituted C6-C30 fused ring heteroaryl.

Preferably, the L² is a single bond, and the Ar² is selected from the group consisting of any of the following substituted or unsubstituted groups: naphthyl, phenanthryl, anthracyl, dibenzofuranyl, dibenzothiophenyl, carbazolyl, 9,9dimethylfluorenyl, 9,9diphenylfluorenyl, spirofluorenyl, triphenylene, fluoranthenyl, benzo9,9dimethylfluorenyl, and benzospirofluorenyl.

Preferably, the L² is a single bond, and the Ar² is selected from the group consisting of any of the following substituted or unsubstituted groups:

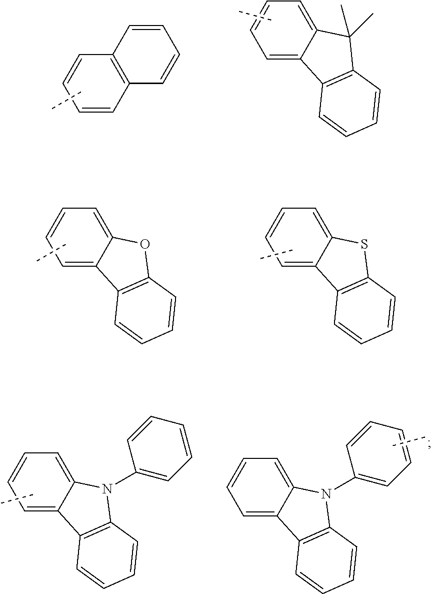

wherein, the dotted line represents a connecting bond of the groups.

In the disclosure, the expression of the ring structure crossed by the dotted line indicates that the connection site is at any position on the ring structure where bonds can be formed.

Preferably, the L² is phenylene, and the Ar² is selected from the group consisting of a substituted or unsubstituted C6-C30 aryl or a substituted or unsubstituted C3-C30 heteroaryl.

Preferably, the L² is phenylene, and the Ar² is selected from the group consisting of any of the following substituted or unsubstituted groups: phenyl, biphenyl, terphenyl, naphthyl, phenanthryl, anthracyl, dibenzofuranyl, dibenzothiophenyl, carbazolyl, 9,9dimethylfluorenyl, 9,9diphenylfluorenyl, spirofluorenyl, triphenylene, fluoranthenyl, benzo9,9dimethylfluorenyl, and benzospirofluorenyl.

Preferably, the L² is phenylene, and the Ar² is selected from the group consisting of any of the following substituted or unsubstituted groups:

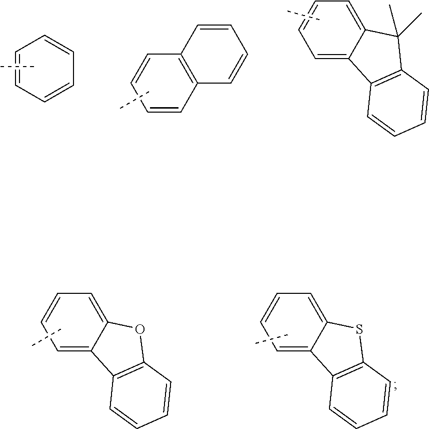

wherein, the dotted line represents a connecting bond of the groups.

Preferably, the R¹, R² and R³ are independently selected from the group consisting of one of methyl, ethyl or phenyl.

Preferably, the R¹, R² and R³ are all methyl.

Preferably, the L¹ is selected from the group consisting of one of the following substituted or unsubstituted groups: phenylene, biphenylene, naphthylene, dibenzofuranylene, dibenzothiophenylene, and 9,9dimethylfluorenylene.

Preferably, the L¹ is selected from the group consisting of any of the following substituted or unsubstituted groups:

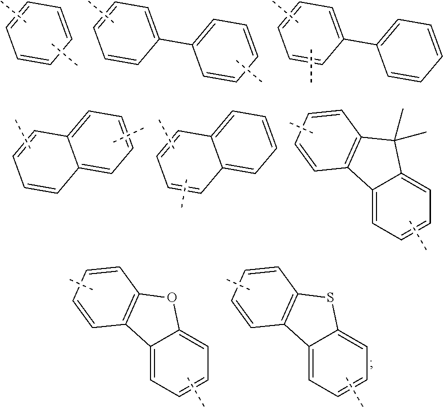

wherein, the dotted line represents a connecting bond of the groups.

Preferably, the m is 0.

Preferably, the compound has anyone of the following structures as shown in P1-P777:

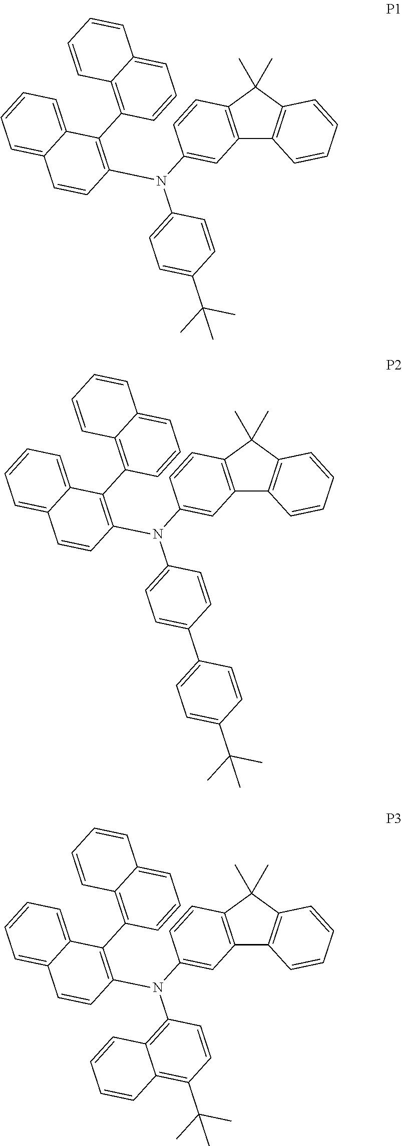 [SMILES: CC(C)(C)c1ccc(N(c2ccc3c(c2)-c2ccccc2C3(C)C)c2ccc3ccccc3c2-c2cccc3ccccc23)cc1 ; CC(C)(C)c1ccc(-c2ccc(N(c3ccc4c(c3)-c3ccccc3C4(C)C)c3ccc4ccccc4c3-c3cccc4ccccc34)cc2)cc1 ; CC(C)(C)c1ccc(N(c2ccc3c(c2)-c2ccccc2C3(C)C)c2ccc3ccccc3c2-c2cccc3ccccc23)c2ccccc12]

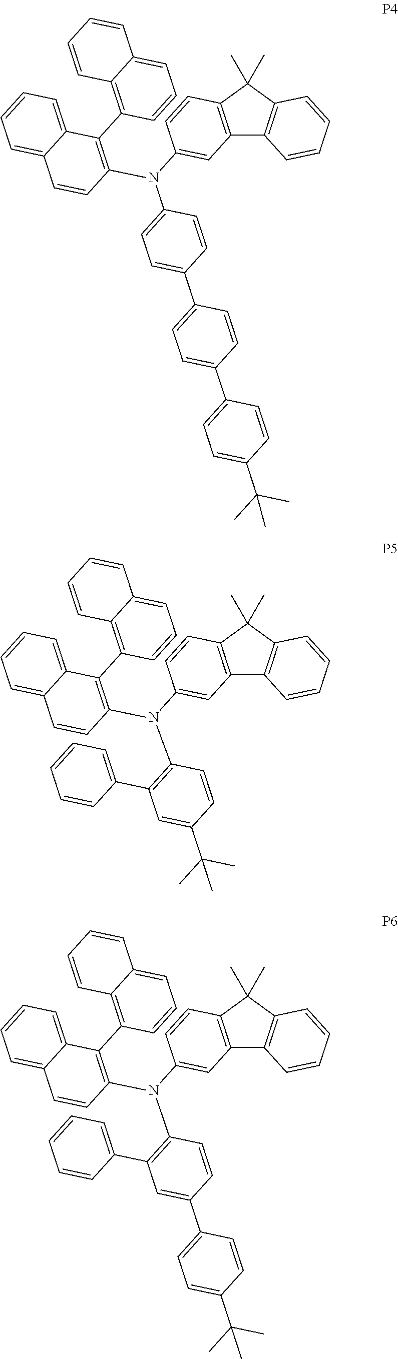 [SMILES: CC(C)(C)c1ccc(-c2ccc(-c3ccc(N(c4ccc5c(c4)-c4ccccc4C5(C)C)c4ccc5ccccc5c4-c4cccc5ccccc45)cc3)cc2)cc1 ; CC(C)(C)c1ccc(N(c2ccc3c(c2)-c2ccccc2C3(C)C)c2ccc3ccccc3c2-c2cccc3ccccc23)c(-c2ccccc2)c1 ; CC(C)(C)c1ccc(-c2ccc(N(c3ccc4c(c3)-c3ccccc3C4(C)C)c3ccc4ccccc4c3-c3cccc4ccccc34)c(-c3ccccc3)c2)cc1]

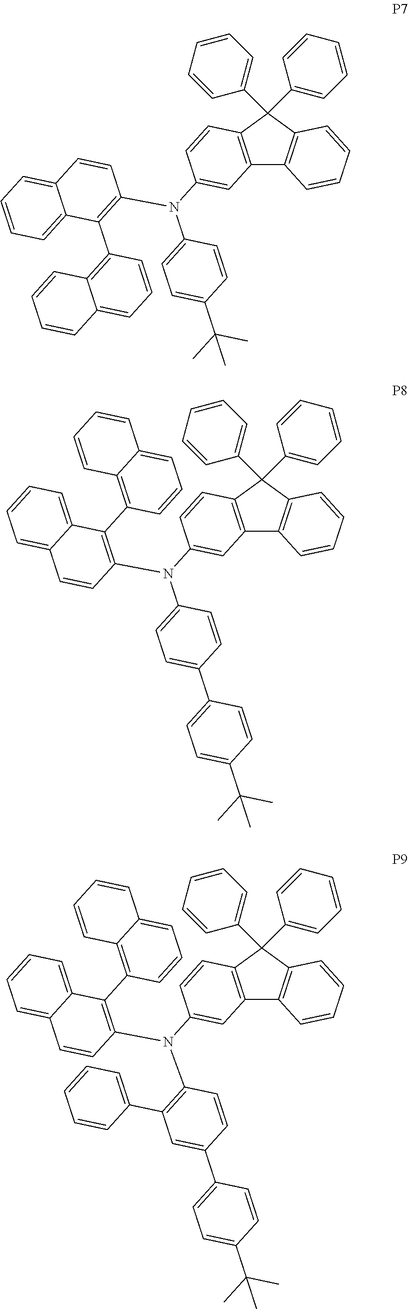

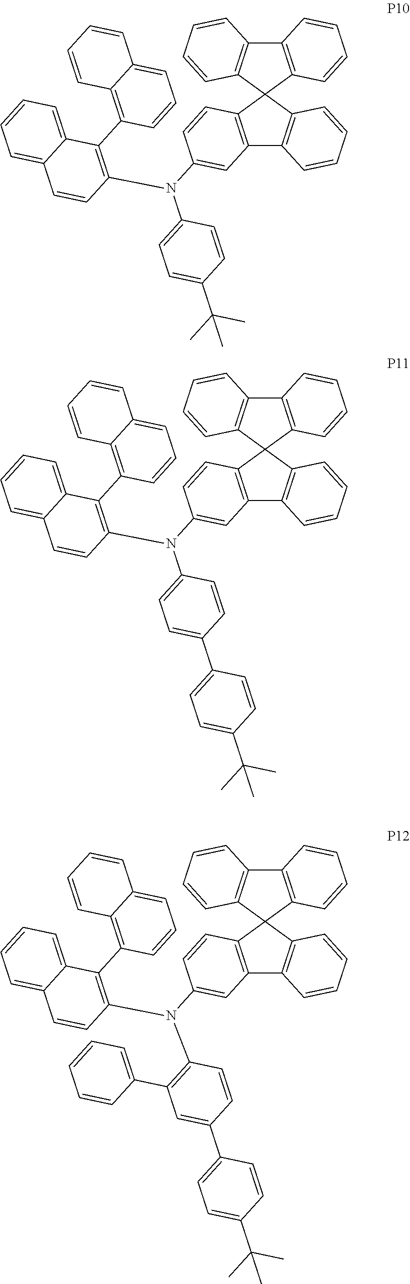

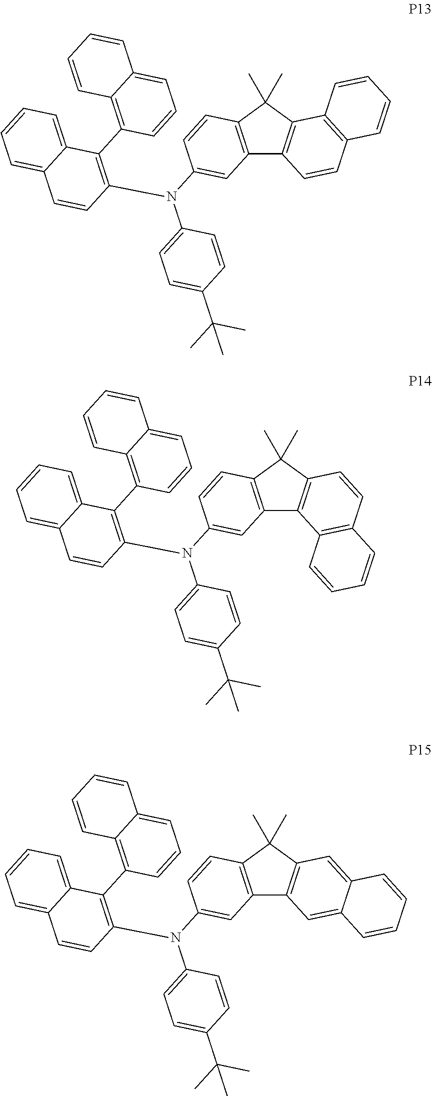 [SMILES: CC(C)(C)c1ccc(N(c2ccc3c(c2)-c2ccc4ccccc4c2C3(C)C)c2ccc3ccccc3c2-c2cccc3ccccc23)cc1 ; CC(C)(C)c1ccc(N(c2ccc3c(c2)-c2c(ccc4ccccc24)C3(C)C)c2ccc3ccccc3c2-c2cccc3ccccc23)cc1 ; CC(C)(C)c1ccc(N(c2ccc3c(c2)-c2cc4ccccc4cc2C3(C)C)c2ccc3ccccc3c2-c2cccc3ccccc23)cc1]

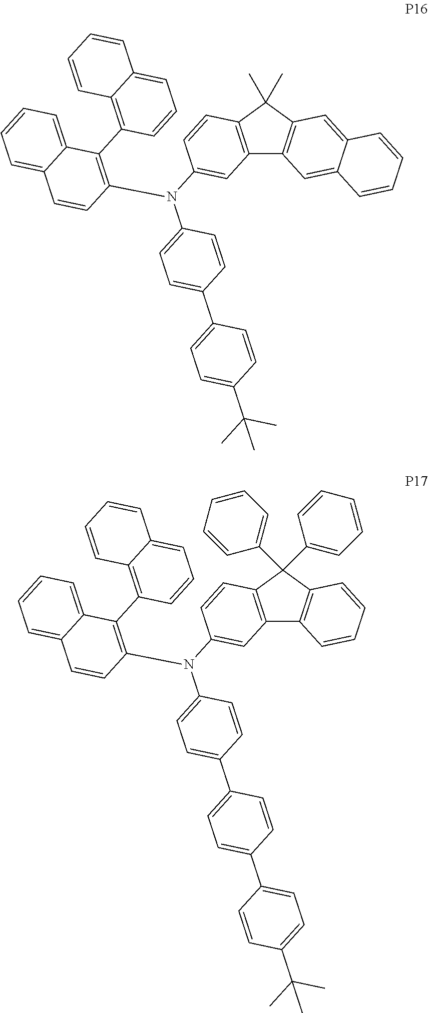

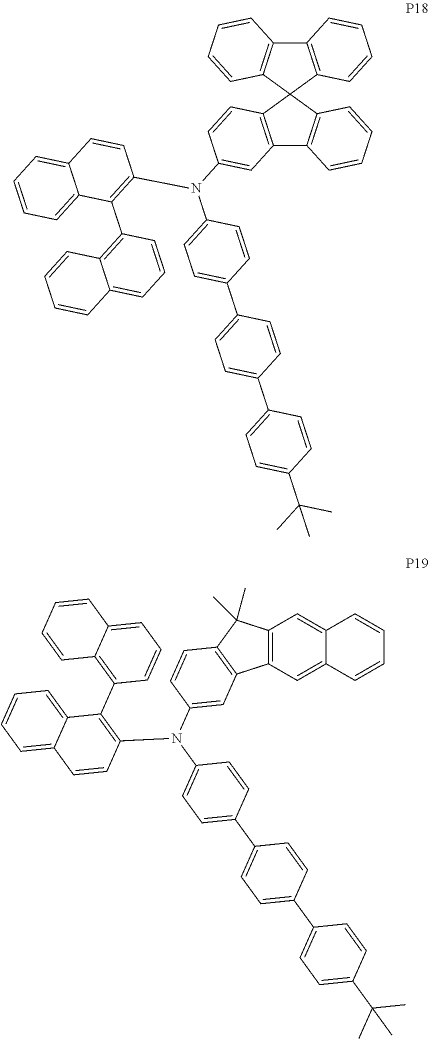 [SMILES: CC(C)(C)c1ccc(-c2ccc(-c3ccc(N(c4ccc5c(c4)-c4ccccc4C54c5ccccc5-c5ccccc54)c4ccc5ccccc5c4-c4cccc5ccccc45)cc3)cc2)cc1 ; CC(C)(C)c1ccc(-c2ccc(-c3ccc(N(c4ccc5c(c4)-c4cc6ccccc6cc4C5(C)C)c4ccc5ccccc5c4-c4cccc5ccccc45)cc3)cc2)cc1]

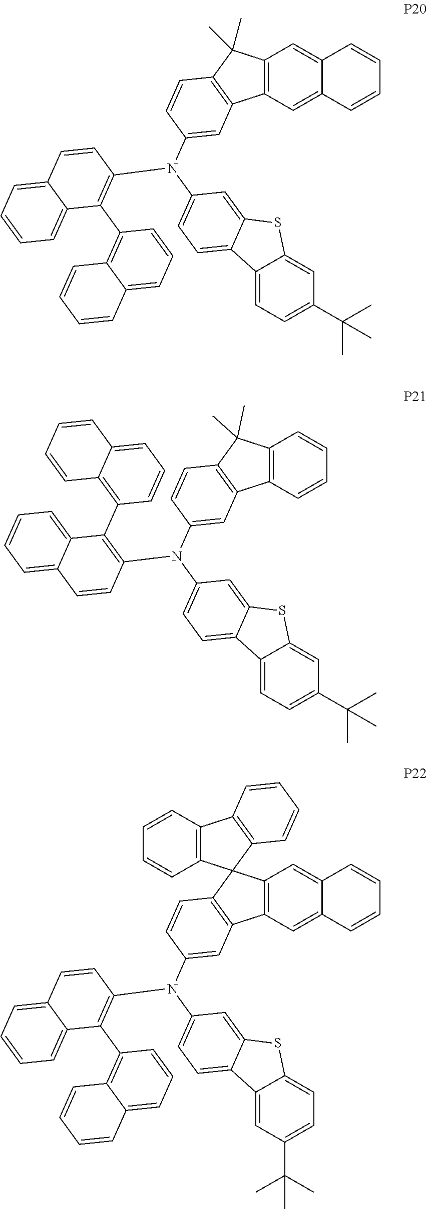 [SMILES: CC(C)(C)c1ccc2c(c1)sc1cc(N(c3ccc4c(c3)-c3cc5ccccc5cc3C4(C)C)c3ccc4ccccc4c3-c3cccc4ccccc34)ccc12 ; CC(C)(C)c1ccc2c(c1)sc1cc(N(c3ccc4c(c3)-c3ccccc3C4(C)C)c3ccc4ccccc4c3-c3cccc4ccccc34)ccc12 ; CC(C)(C)c1ccc2sc3cc(N(c4ccc5c(c4)-c4cc6ccccc6cc4C54c5ccccc5-c5ccccc54)c4ccc5ccccc5c4-c4cccc5ccccc45)ccc3c2c1]

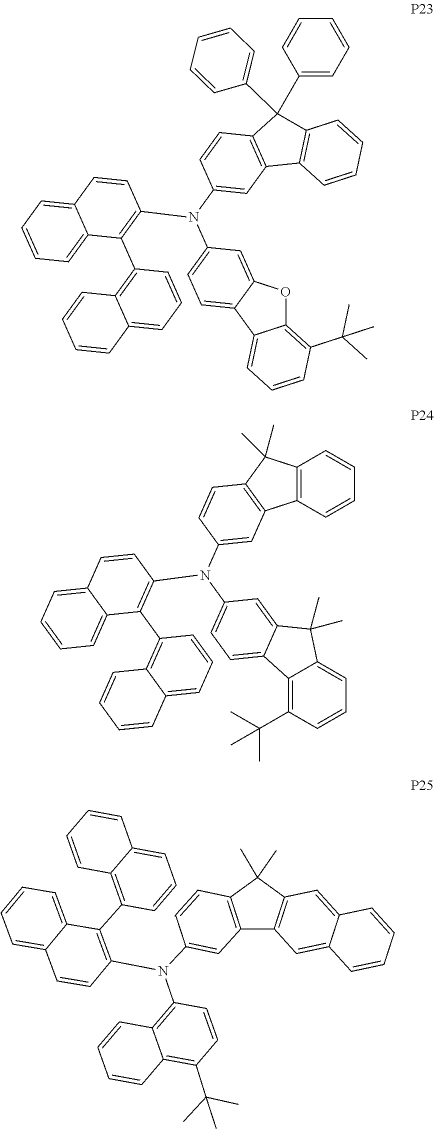

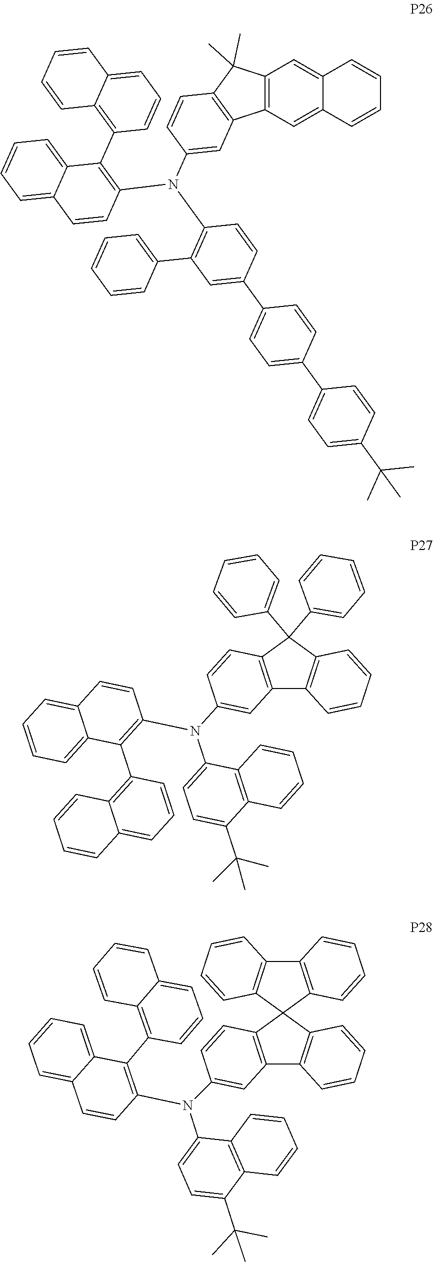 [SMILES: CC(C)(C)c1ccc(-c2ccc(-c3ccc(N(c4ccc5c(c4)-c4cc6ccccc6cc4C5(C)C)c4ccc5ccccc5c4-c4cccc5ccccc45)c(-c4ccccc4)c3)cc2)cc1 ; CC(C)(C)c1ccc(N(c2ccc3c(c2)-c2ccccc2C3(c2ccccc2)c2ccccc2)c2ccc3ccccc3c2-c2cccc3ccccc23)c2ccccc12 ; CC(C)(C)c1ccc(N(c2ccc3c(c2)-c2ccccc2C32c3ccccc3-c3ccccc32)c2ccc3ccccc3c2-c2cccc3ccccc23)c2ccccc12]

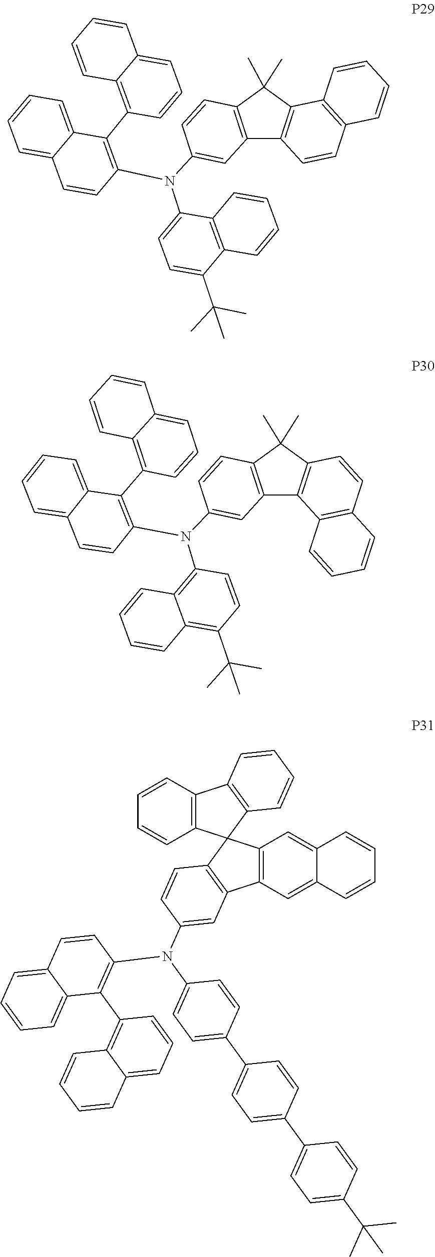 [SMILES: CC(C)(C)c1ccc(N(c2ccc3c(c2)-c2ccc4ccccc4c2C3(C)C)c2ccc3ccccc3c2-c2cccc3ccccc23)c2ccccc12 ; CC(C)(C)c1ccc(N(c2ccc3c(c2)-c2c(ccc4ccccc24)C3(C)C)c2ccc3ccccc3c2-c2cccc3ccccc23)c2ccccc12 ; CC(C)(C)c1ccc(-c2ccc(-c3ccc(N(c4ccc5c(c4)-c4cc6ccccc6cc4C54c5ccccc5-c5ccccc54)c4ccc5ccccc5c4-c4cccc5ccccc45)cc3)cc2)cc1]

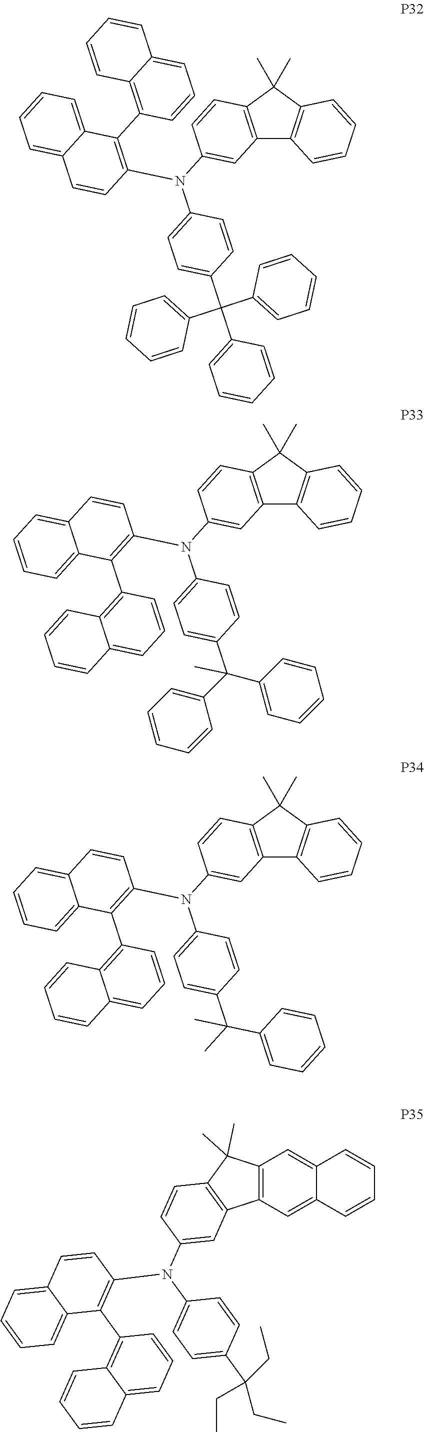

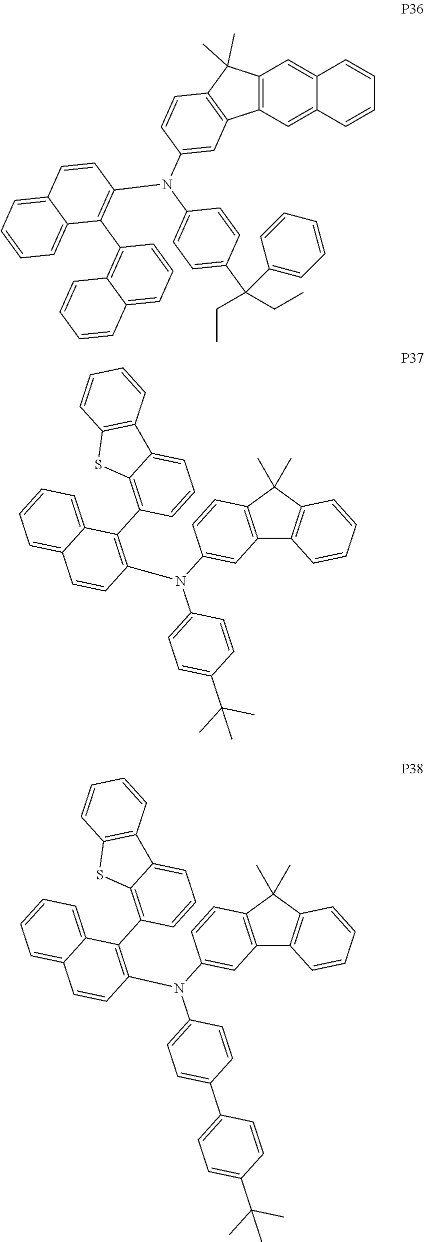

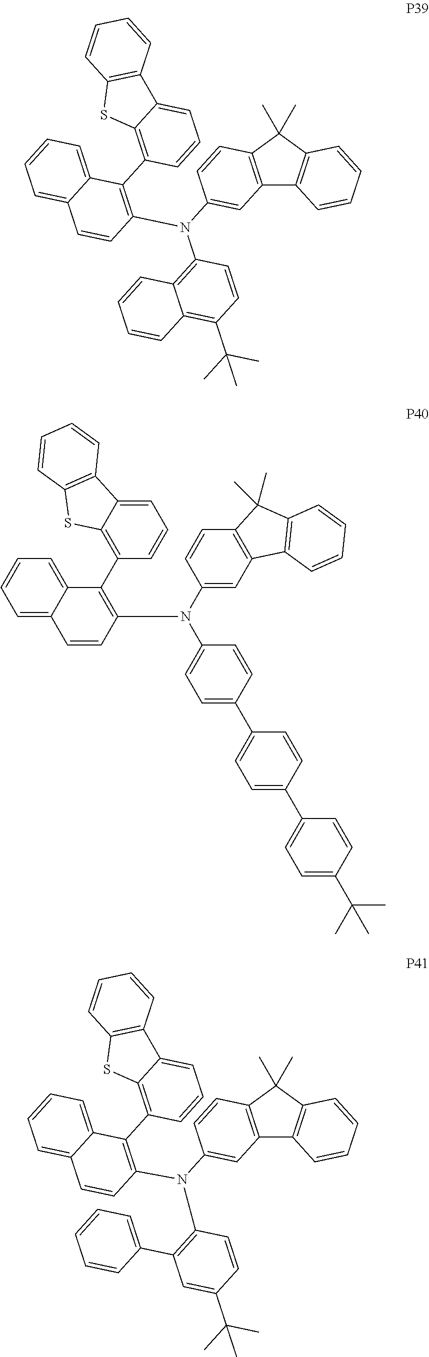 [SMILES: CC(C)(C)c1ccc(N(c2ccc3c(c2)-c2ccccc2C3(C)C)c2ccc3ccccc3c2-c2cccc3c2sc2ccccc23)c2ccccc12 ; CC(C)(C)c1ccc(-c2ccc(-c3ccc(N(c4ccc5c(c4)-c4ccccc4C5(C)C)c4ccc5ccccc5c4-c4cccc5c4sc4ccccc45)cc3)cc2)cc1 ; CC(C)(C)c1ccc(N(c2ccc3c(c2)-c2ccccc2C3(C)C)c2ccc3ccccc3c2-c2cccc3c2sc2ccccc23)c(-c2ccccc2)c1]

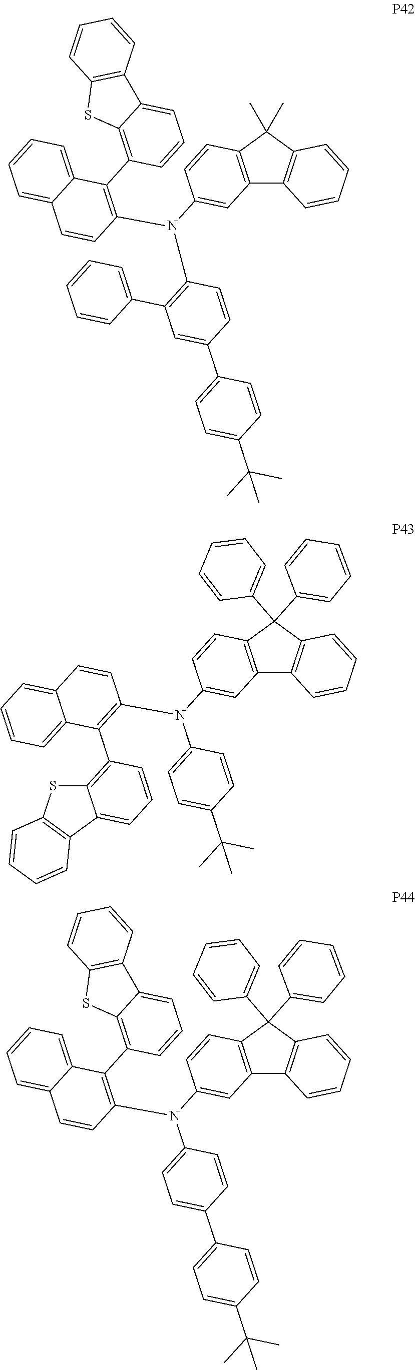

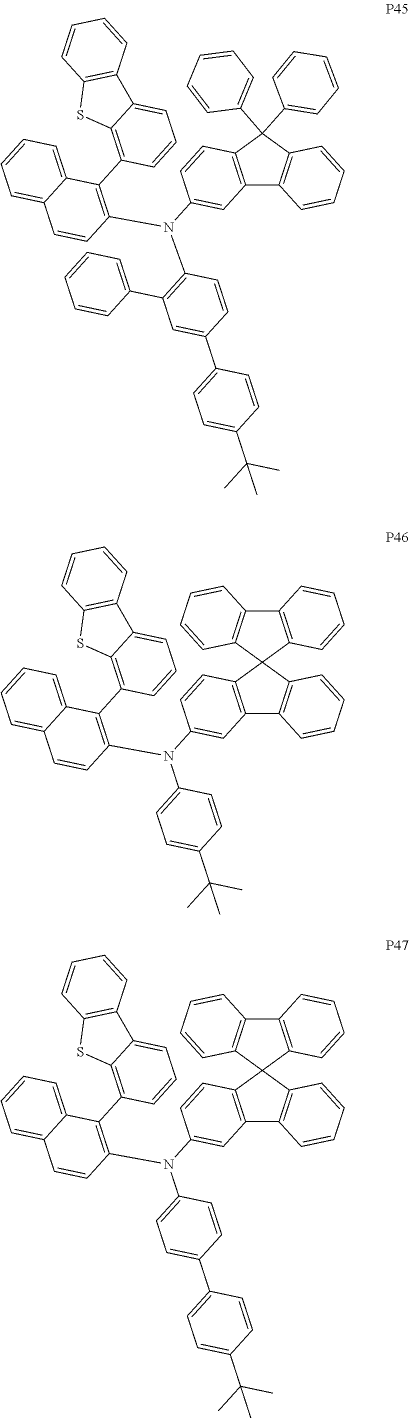 [SMILES: CC(C)(C)c1ccc(-c2ccc(N(c3ccc4c(c3)-c3ccccc3C4(c3ccccc3)c3ccccc3)c3ccc4ccccc4c3-c3cccc4c3sc3ccccc34)c(-c3ccccc3)c2)cc1 ; CC(C)(C)c1ccc(N(c2ccc3c(c2)-c2ccccc2C32c3ccccc3-c3ccccc32)c2ccc3ccccc3c2-c2cccc3c2sc2ccccc23)cc1 ; CC(C)(C)c1ccc(-c2ccc(N(c3ccc4c(c3)-c3ccccc3C43c4ccccc4-c4ccccc43)c3ccc4ccccc4c3-c3cccc4c3sc3ccccc34)cc2)cc1]

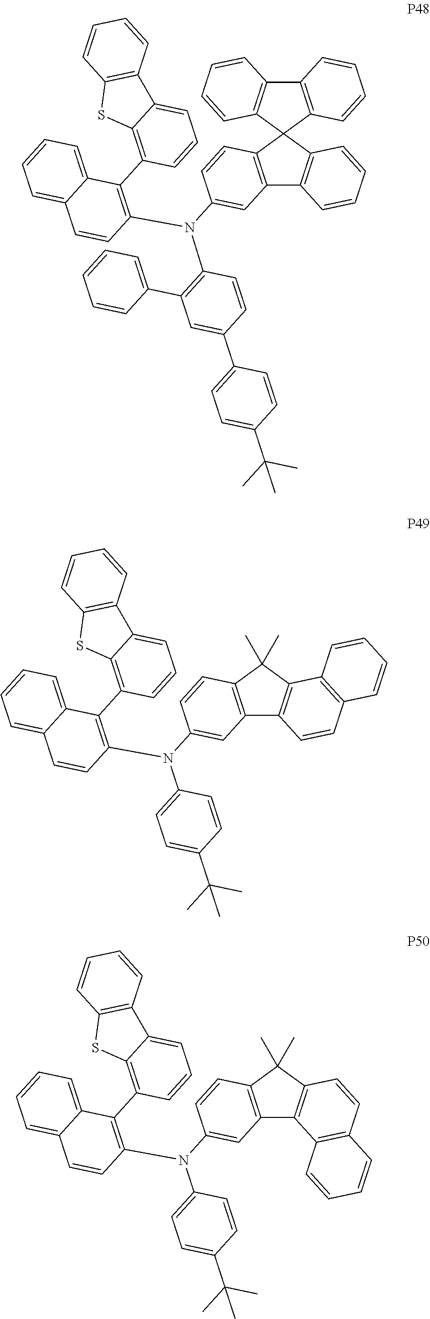 [SMILES: CC(C)(C)c1ccc(-c2ccc(N(c3ccc4c(c3)-c3ccccc3C43c4ccccc4-c4ccccc43)c3ccc4ccccc4c3-c3cccc4c3sc3ccccc34)c(-c3ccccc3)c2)cc1 ; CC(C)(C)c1ccc(N(c2ccc3c(c2)-c2ccc4ccccc4c2C3(C)C)c2ccc3ccccc3c2-c2cccc3c2sc2ccccc23)cc1 ; CC(C)(C)c1ccc(N(c2ccc3c(c2)-c2c(ccc4ccccc24)C3(C)C)c2ccc3ccccc3c2-c2cccc3c2sc2ccccc23)cc1]

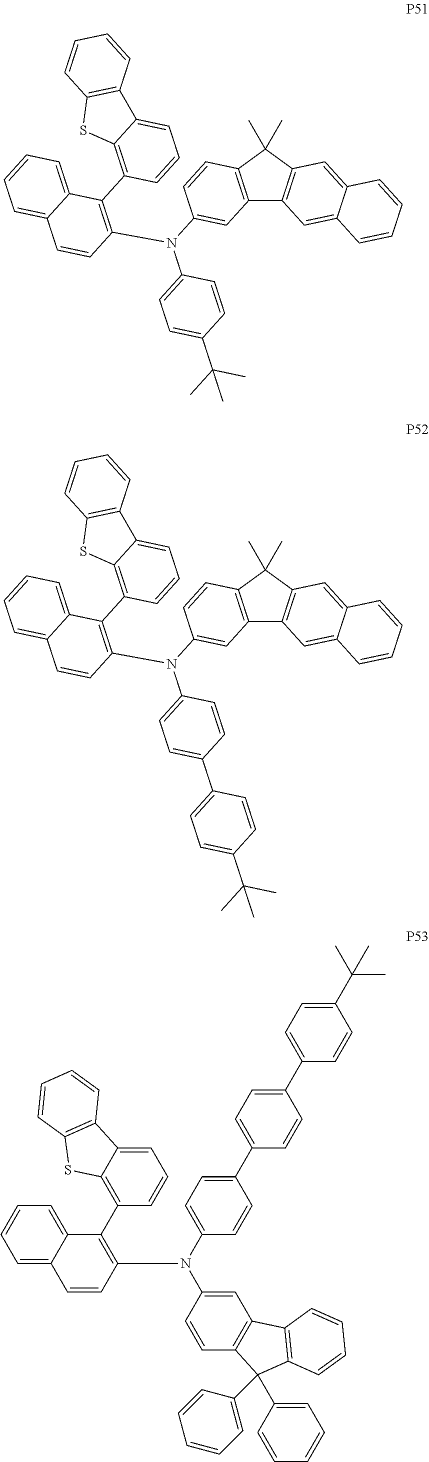 [SMILES: CC(C)(C)c1ccc(N(c2ccc3c(c2)-c2cc4ccccc4cc2C3(C)C)c2ccc3ccccc3c2-c2cccc3c2sc2ccccc23)cc1 ; CC(C)(C)c1ccc(-c2ccc(N(c3ccc4c(c3)-c3cc5ccccc5cc3C4(C)C)c3ccc4ccccc4c3-c3cccc4c3sc3ccccc34)cc2)cc1 ; CC(C)(C)c1ccc(-c2ccc(-c3ccc(N(c4ccc5c(c4)-c4ccccc4C5(c4ccccc4)c4ccccc4)c4ccc5ccccc5c4-c4cccc5c4sc4ccccc45)cc3)cc2)cc1]

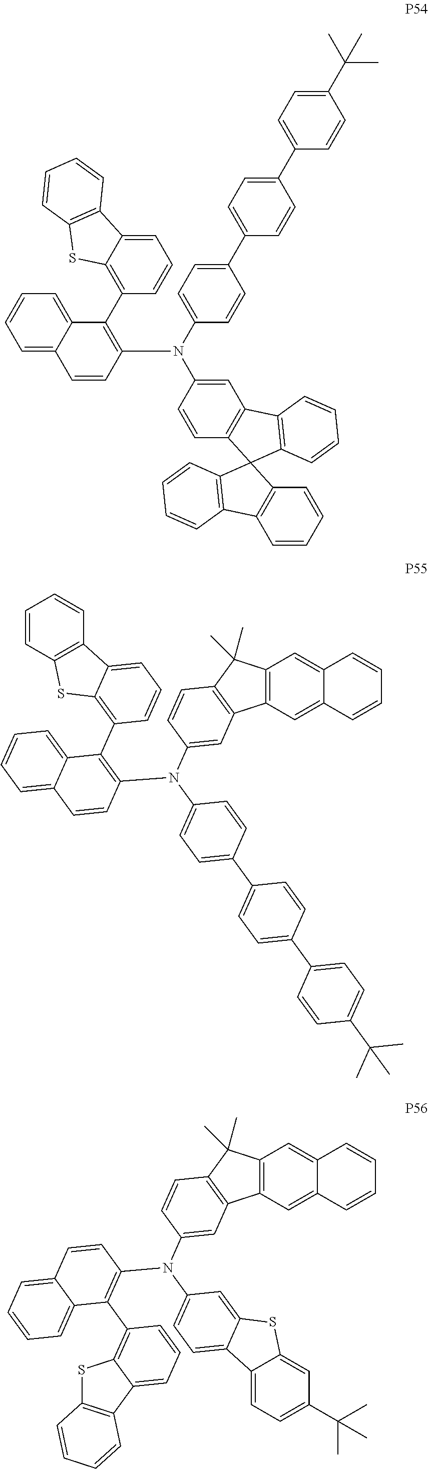 [SMILES: CC(C)(C)c1ccc(-c2ccc(-c3ccc(N(c4ccc5c(c4)-c4ccccc4C54c5ccccc5-c5ccccc54)c4ccc5ccccc5c4-c4cccc5c4sc4ccccc45)cc3)cc2)cc1 ; CC(C)(C)c1ccc(-c2ccc(-c3ccc(N(c4ccc5c(c4)-c4cc6ccccc6cc4C5(C)C)c4ccc5ccccc5c4-c4cccc5c4sc4ccccc45)cc3)cc2)cc1 ; CC(C)(C)c1ccc2c(c1)sc1cc(N(c3ccc4c(c3)-c3cc5ccccc5cc3C4(C)C)c3ccc4ccccc4c3-c3cccc4c3sc3ccccc34)ccc12]

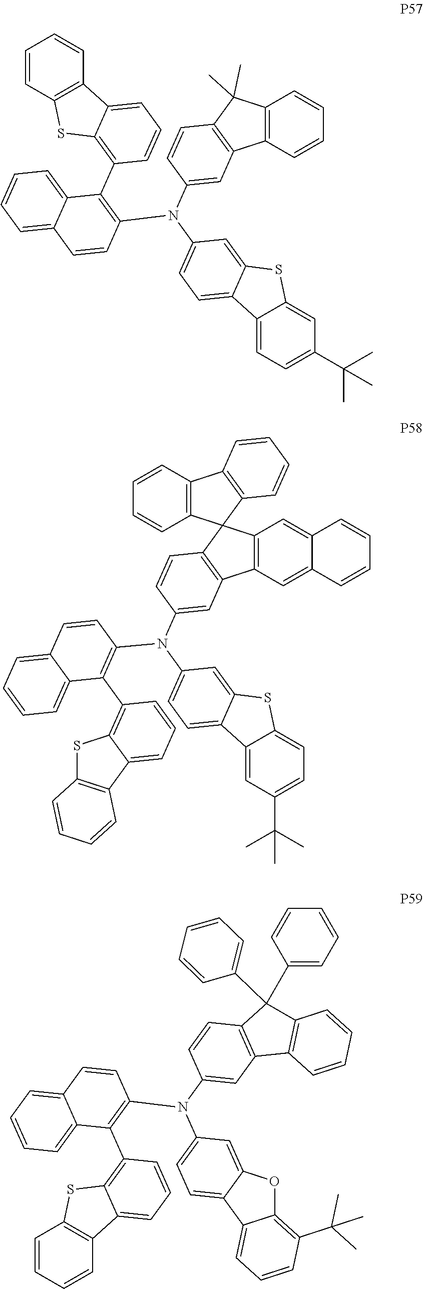

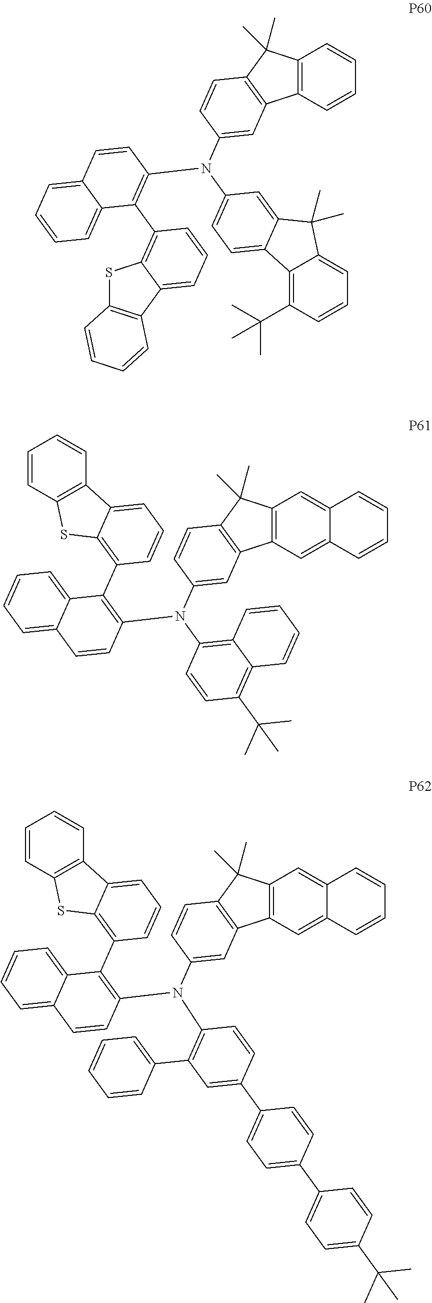 [SMILES: CC(C)(C)c1cccc2c1-c1ccc(N(c3ccc4c(c3)-c3ccccc3C4(C)C)c3ccc4ccccc4c3-c3cccc4c3sc3ccccc34)cc1C2(C)C ; CC(C)(C)c1ccc(N(c2ccc3c(c2)-c2cc4ccccc4cc2C3(C)C)c2ccc3ccccc3c2-c2cccc3c2sc2ccccc23)c2ccccc12 ; CC(C)(C)c1ccc(-c2ccc(-c3ccc(N(c4ccc5c(c4)-c4cc6ccccc6cc4C5(C)C)c4ccc5ccccc5c4-c4cccc5c4sc4ccccc45)c(-c4ccccc4)c3)cc2)cc1]

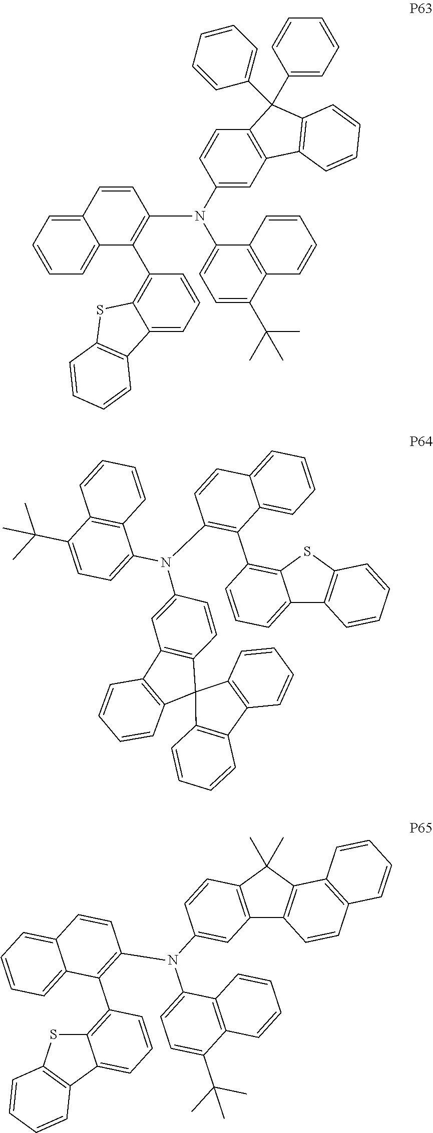 [SMILES: CC(C)(C)c1ccc(N(c2ccc3c(c2)-c2ccccc2C3(c2ccccc2)c2ccccc2)c2ccc3ccccc3c2-c2cccc3c2sc2ccccc23)c2ccccc12 ; CC(C)(C)c1ccc(N(c2ccc3c(c2)-c2ccccc2C32c3ccccc3-c3ccccc32)c2ccc3ccccc3c2-c2cccc3c2sc2ccccc23)c2ccccc12 ; CC(C)(C)c1ccc(N(c2ccc3c(c2)-c2ccc4ccccc4c2C3(C)C)c2ccc3ccccc3c2-c2cccc3c2sc2ccccc23)c2ccccc12]

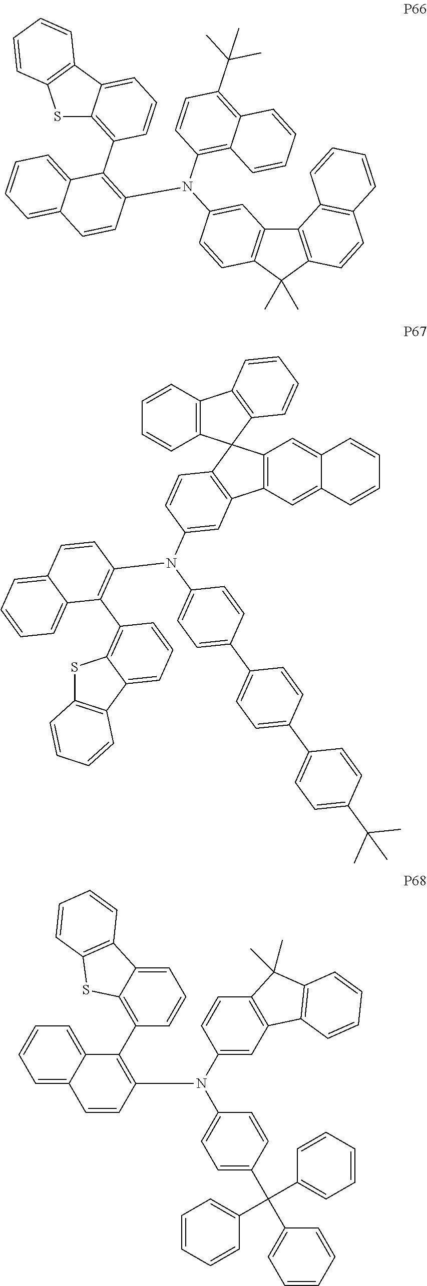 [SMILES: CC(C)(C)c1ccc(N(c2ccc3c(c2)-c2c(ccc4ccccc24)C3(C)C)c2ccc3ccccc3c2-c2cccc3c2sc2ccccc23)c2ccccc12 ; CC(C)(C)c1ccc(-c2ccc(-c3ccc(N(c4ccc5c(c4)-c4cc6ccccc6cc4C54c5ccccc5-c5ccccc54)c4ccc5ccccc5c4-c4cccc5c4sc4ccccc45)cc3)cc2)cc1 ; CC1(C)c2ccccc2-c2cc(N(c3ccc(C(c4ccccc4)(c4ccccc4)c4ccccc4)cc3)c3ccc4ccccc4c3-c3cccc4c3sc3ccccc34)ccc21]

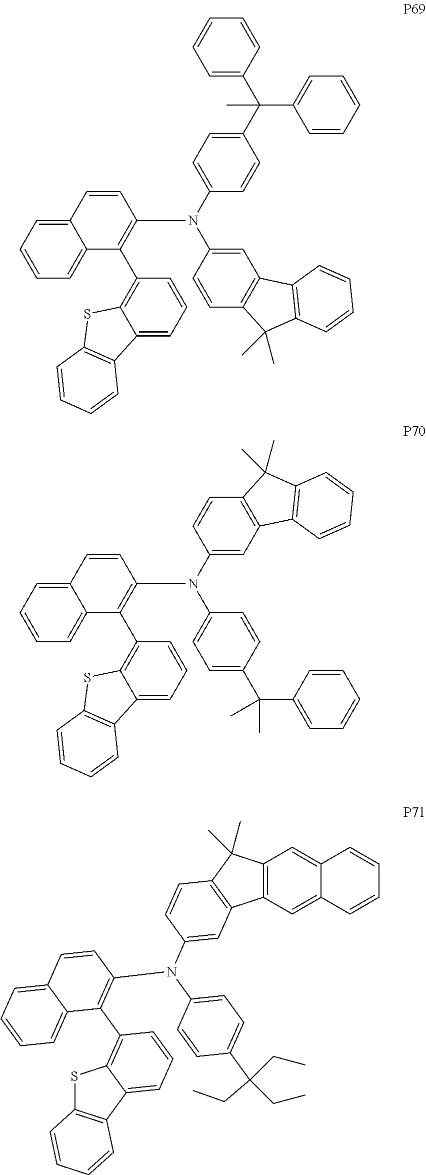 [SMILES: CC1(C)c2ccccc2-c2cc(N(c3ccc(C(C)(c4ccccc4)c4ccccc4)cc3)c3ccc4ccccc4c3-c3cccc4c3sc3ccccc34)ccc21 ; CC(C)(c1ccccc1)c1ccc(N(c2ccc3c(c2)-c2ccccc2C3(C)C)c2ccc3ccccc3c2-c2cccc3c2sc2ccccc23)cc1 ; CCC(CC)(CC)c1ccc(N(c2ccc3c(c2)-c2cc4ccccc4cc2C3(C)C)c2ccc3ccccc3c2-c2cccc3c2sc2ccccc23)cc1]

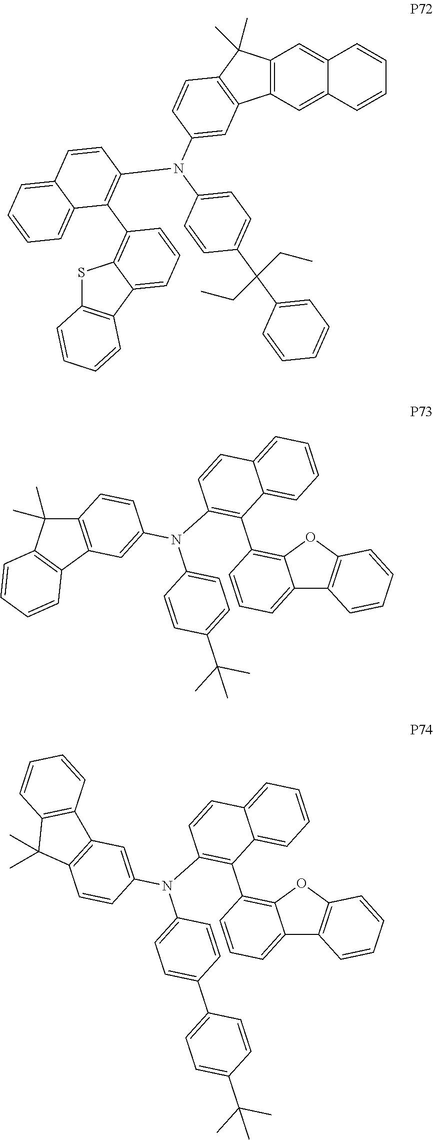 [SMILES: CCC(CC)(c1ccccc1)c1ccc(N(c2ccc3c(c2)-c2cc4ccccc4cc2C3(C)C)c2ccc3ccccc3c2-c2cccc3c2sc2ccccc23)cc1 ; CC(C)(C)c1ccc(N(c2ccc3c(c2)-c2ccccc2C3(C)C)c2ccc3ccccc3c2-c2cccc3c2oc2ccccc23)cc1 ; CC(C)(C)c1ccc(-c2ccc(N(c3ccc4c(c3)-c3ccccc3C4(C)C)c3ccc4ccccc4c3-c3cccc4c3oc3ccccc34)cc2)cc1]

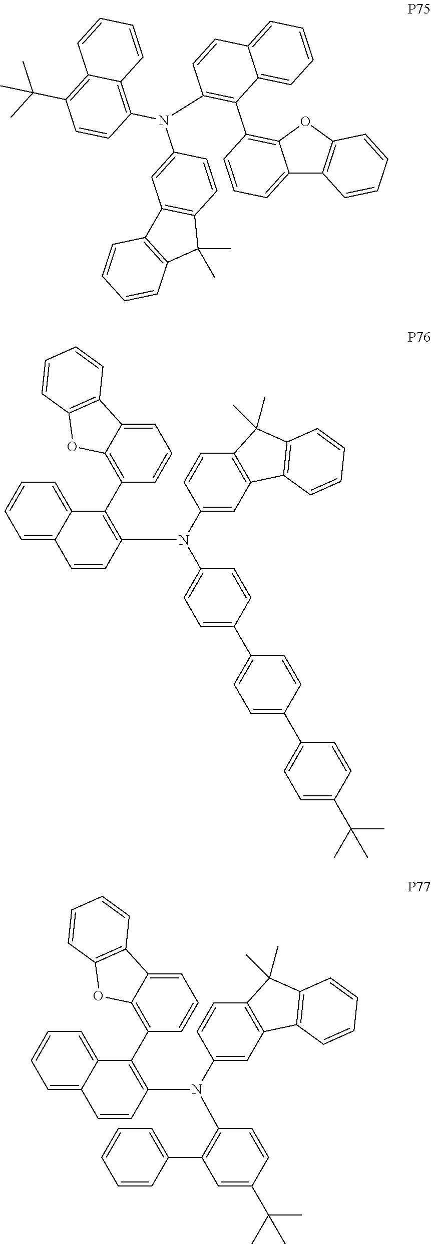 [SMILES: CC(C)(C)c1ccc(N(c2ccc3c(c2)-c2ccccc2C3(C)C)c2ccc3ccccc3c2-c2cccc3c2oc2ccccc23)c2ccccc12 ; CC(C)(C)c1ccc(-c2ccc(-c3ccc(N(c4ccc5c(c4)-c4ccccc4C5(C)C)c4ccc5ccccc5c4-c4cccc5c4oc4ccccc45)cc3)cc2)cc1 ; CC(C)(C)c1ccc(N(c2ccc3c(c2)-c2ccccc2C3(C)C)c2ccc3ccccc3c2-c2cccc3c2oc2ccccc23)c(-c2ccccc2)c1]

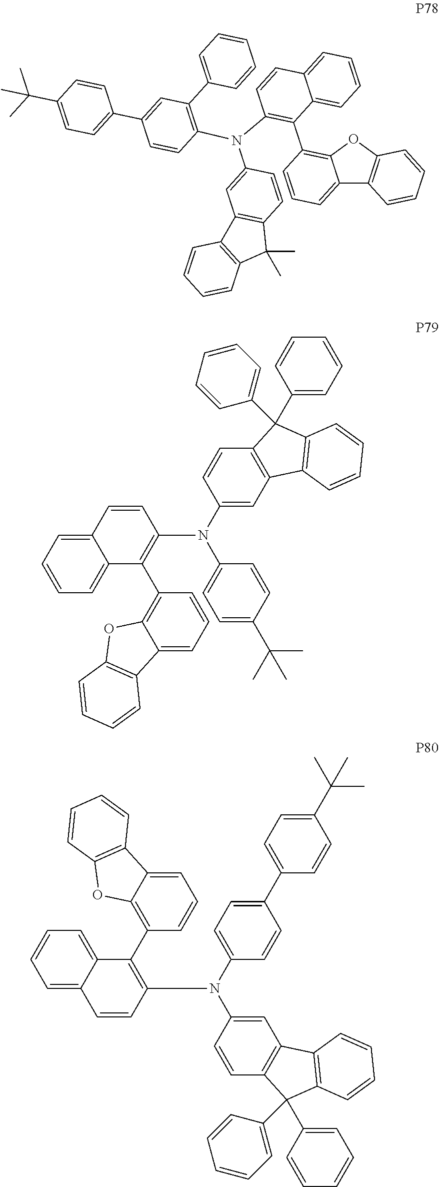 [SMILES: CC(C)(C)c1ccc(-c2ccc(N(c3ccc4c(c3)-c3ccccc3C4(C)C)c3ccc4ccccc4c3-c3cccc4c3oc3ccccc34)c(-c3ccccc3)c2)cc1 ; CC(C)(C)c1ccc(N(c2ccc3c(c2)-c2ccccc2C3(c2ccccc2)c2ccccc2)c2ccc3ccccc3c2-c2cccc3c2oc2ccccc23)cc1 ; CC(C)(C)c1ccc(-c2ccc(N(c3ccc4c(c3)-c3ccccc3C4(c3ccccc3)c3ccccc3)c3ccc4ccccc4c3-c3cccc4c3oc3ccccc34)cc2)cc1]

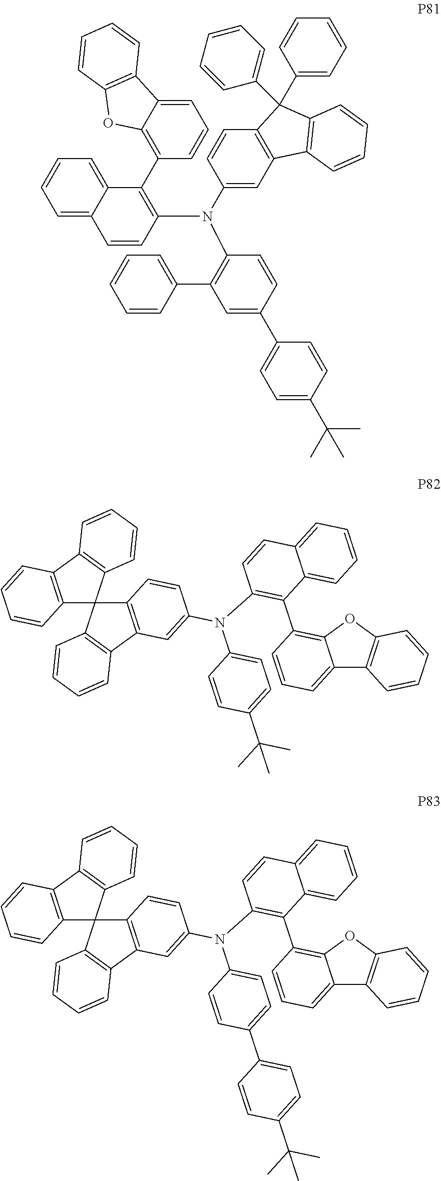 [SMILES: CC(C)(C)c1ccc(-c2ccc(N(c3ccc4c(c3)-c3ccccc3C4(c3ccccc3)c3ccccc3)c3ccc4ccccc4c3-c3cccc4c3oc3ccccc34)c(-c3ccccc3)c2)cc1 ; CC(C)(C)c1ccc(N(c2ccc3c(c2)-c2ccccc2C32c3ccccc3-c3ccccc32)c2ccc3ccccc3c2-c2cccc3c2oc2ccccc23)cc1 ; CC(C)(C)c1ccc(-c2ccc(N(c3ccc4c(c3)-c3ccccc3C43c4ccccc4-c4ccccc43)c3ccc4ccccc4c3-c3cccc4c3oc3ccccc34)cc2)cc1]

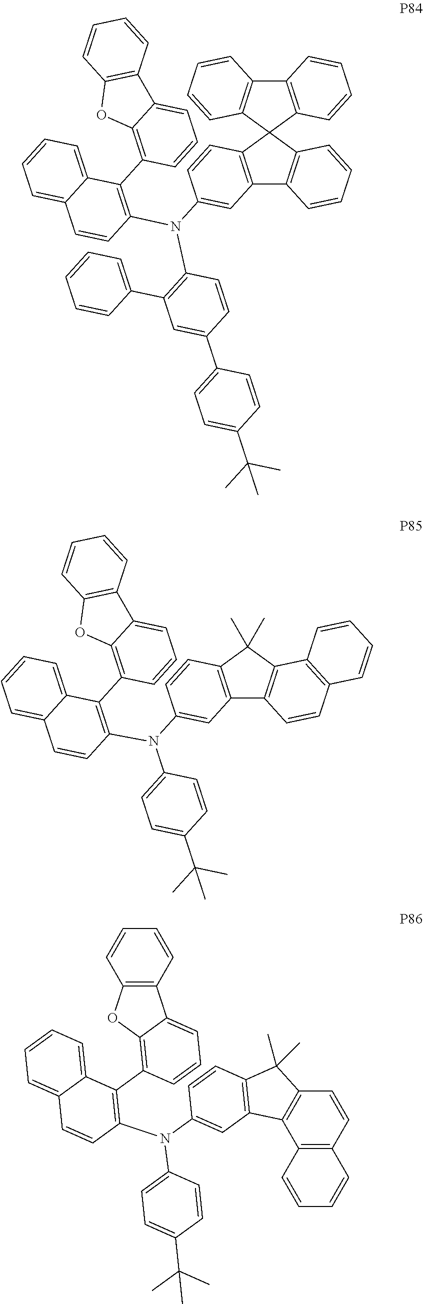

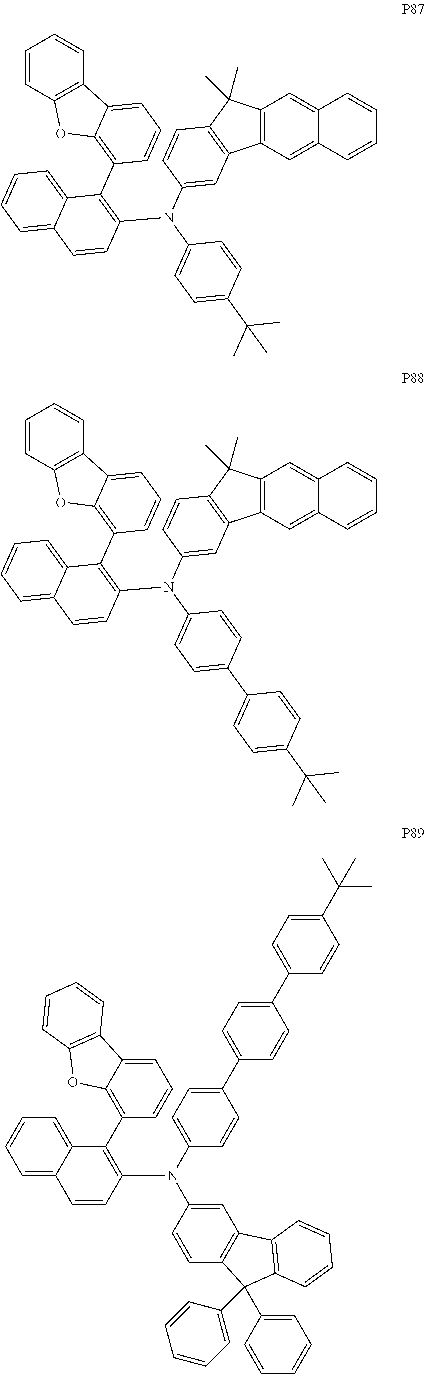 [SMILES: CC(C)(C)c1ccc(N(c2ccc3c(c2)-c2cc4ccccc4cc2C3(C)C)c2ccc3ccccc3c2-c2cccc3c2oc2ccccc23)cc1 ; CC(C)(C)c1ccc(-c2ccc(N(c3ccc4c(c3)-c3cc5ccccc5cc3C4(C)C)c3ccc4ccccc4c3-c3cccc4c3oc3ccccc34)cc2)cc1 ; CC(C)(C)c1ccc(-c2ccc(-c3ccc(N(c4ccc5c(c4)-c4ccccc4C5(c4ccccc4)c4ccccc4)c4ccc5ccccc5c4-c4cccc5c4oc4ccccc45)cc3)cc2)cc1]

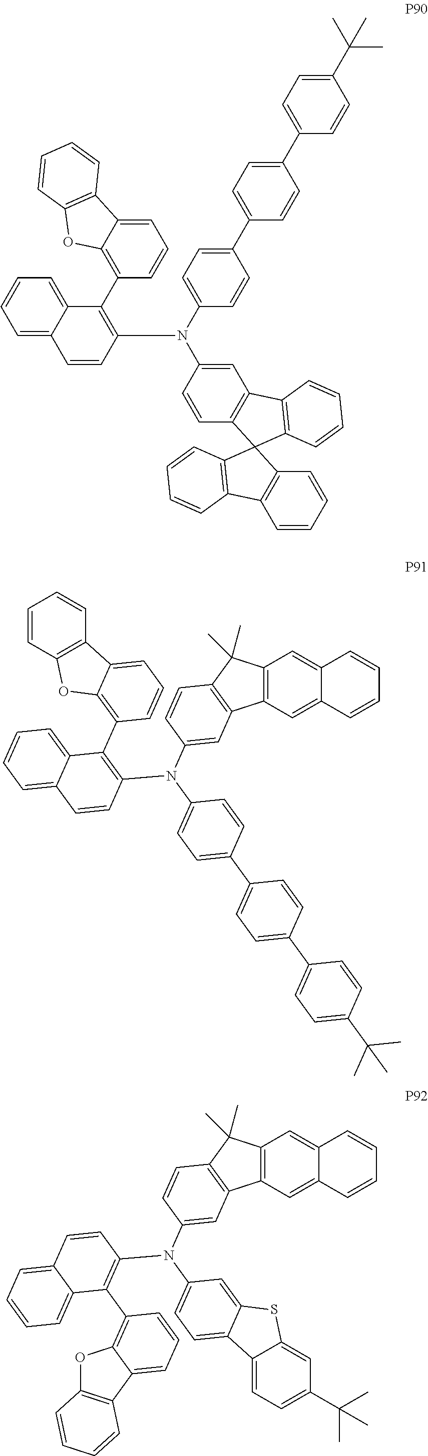 [SMILES: CC(C)(C)c1ccc(-c2ccc(-c3ccc(N(c4ccc5c(c4)-c4ccccc4C54c5ccccc5-c5ccccc54)c4ccc5ccccc5c4-c4cccc5c4oc4ccccc45)cc3)cc2)cc1 ; CC(C)(C)c1ccc(-c2ccc(-c3ccc(N(c4ccc5c(c4)-c4cc6ccccc6cc4C5(C)C)c4ccc5ccccc5c4-c4cccc5c4oc4ccccc45)cc3)cc2)cc1 ; CC(C)(C)c1ccc2c(c1)sc1cc(N(c3ccc4c(c3)-c3cc5ccccc5cc3C4(C)C)c3ccc4ccccc4c3-c3cccc4c3oc3ccccc34)ccc12]

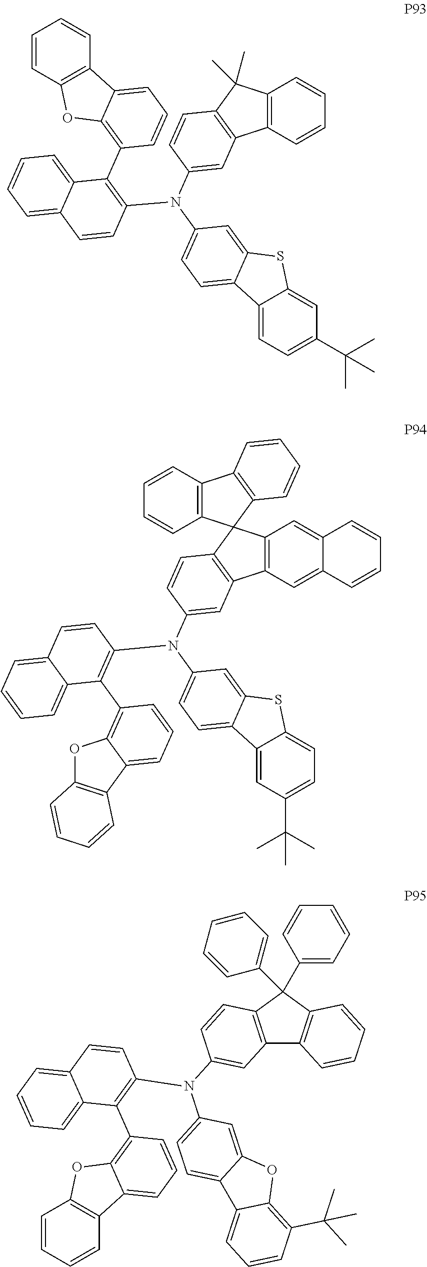

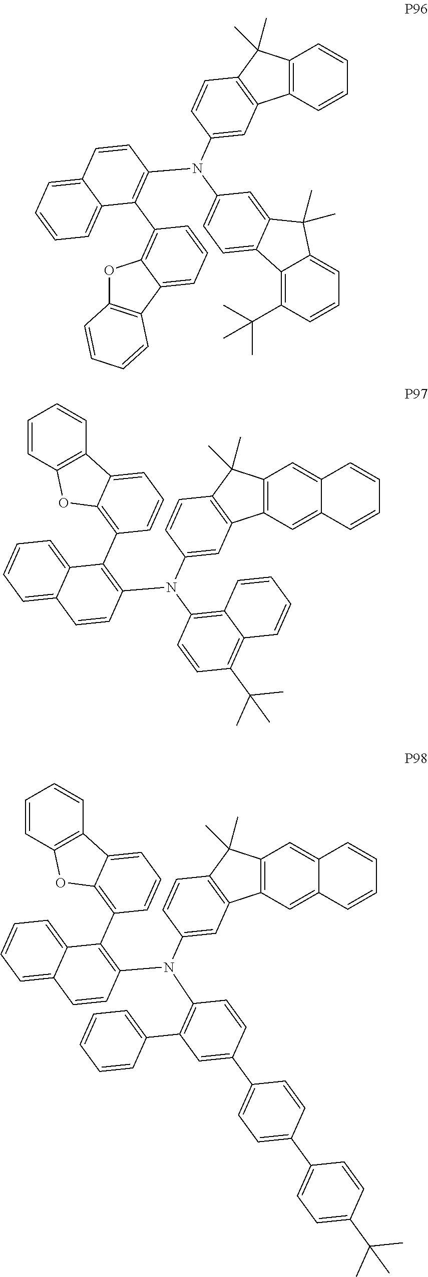 [SMILES: CC(C)(C)c1cccc2c1-c1ccc(N(c3ccc4c(c3)-c3ccccc3C4(C)C)c3ccc4ccccc4c3-c3cccc4c3oc3ccccc34)cc1C2(C)C ; CC(C)(C)c1ccc(N(c2ccc3c(c2)-c2cc4ccccc4cc2C3(C)C)c2ccc3ccccc3c2-c2cccc3c2oc2ccccc23)c2ccccc12 ; CC(C)(C)c1ccc(-c2ccc(-c3ccc(N(c4ccc5c(c4)-c4cc6ccccc6cc4C5(C)C)c4ccc5ccccc5c4-c4cccc5c4oc4ccccc45)c(-c4ccccc4)c3)cc2)cc1]

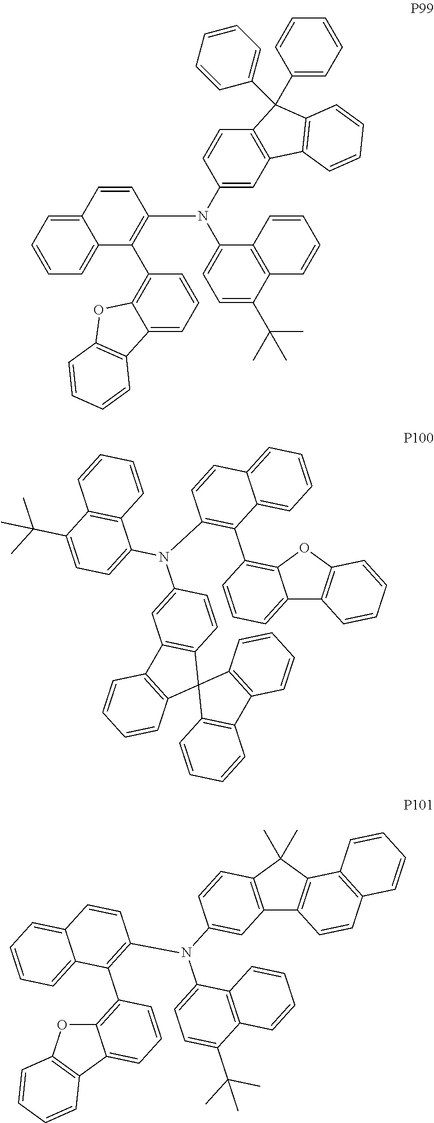

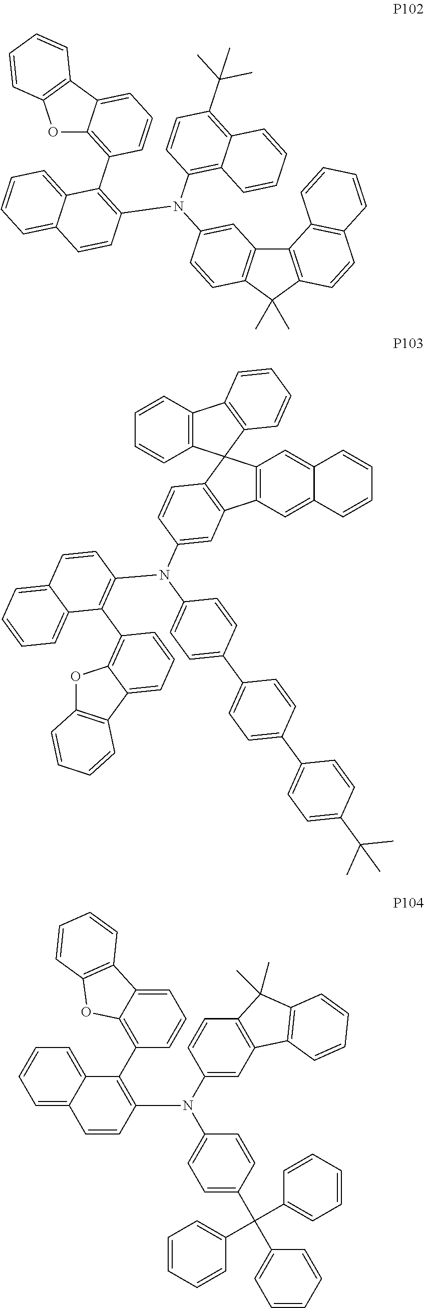

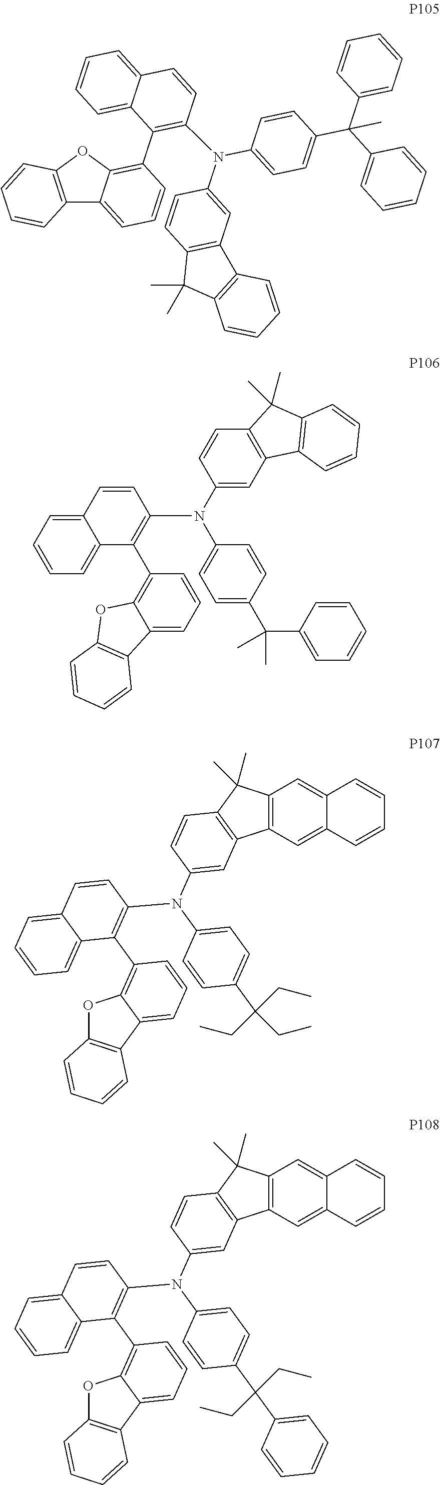

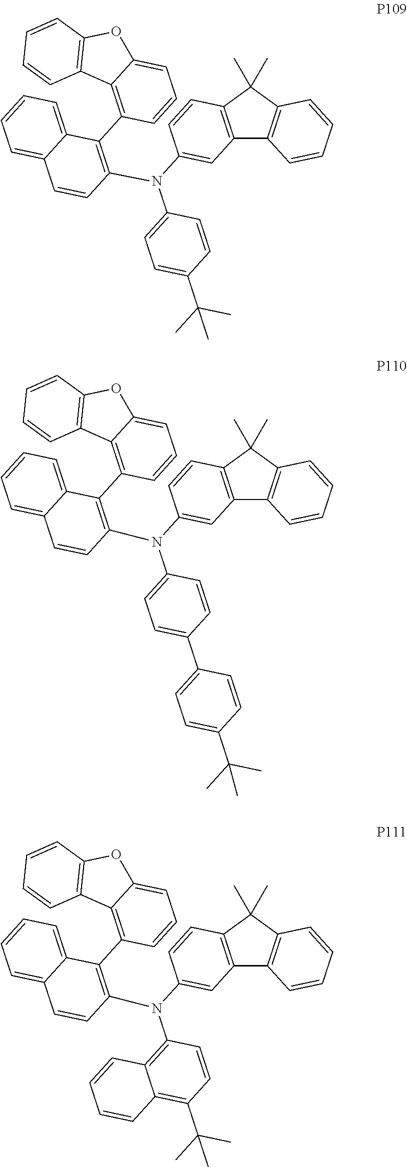 [SMILES: CC(C)(C)c1ccc(N(c2ccc3c(c2)-c2ccccc2C3(C)C)c2ccc3ccccc3c2-c2cccc3oc4ccccc4c23)cc1 ; CC(C)(C)c1ccc(-c2ccc(N(c3ccc4c(c3)-c3ccccc3C4(C)C)c3ccc4ccccc4c3-c3cccc4oc5ccccc5c34)cc2)cc1 ; CC(C)(C)c1ccc(N(c2ccc3c(c2)-c2ccccc2C3(C)C)c2ccc3ccccc3c2-c2cccc3oc4ccccc4c23)c2ccccc12]

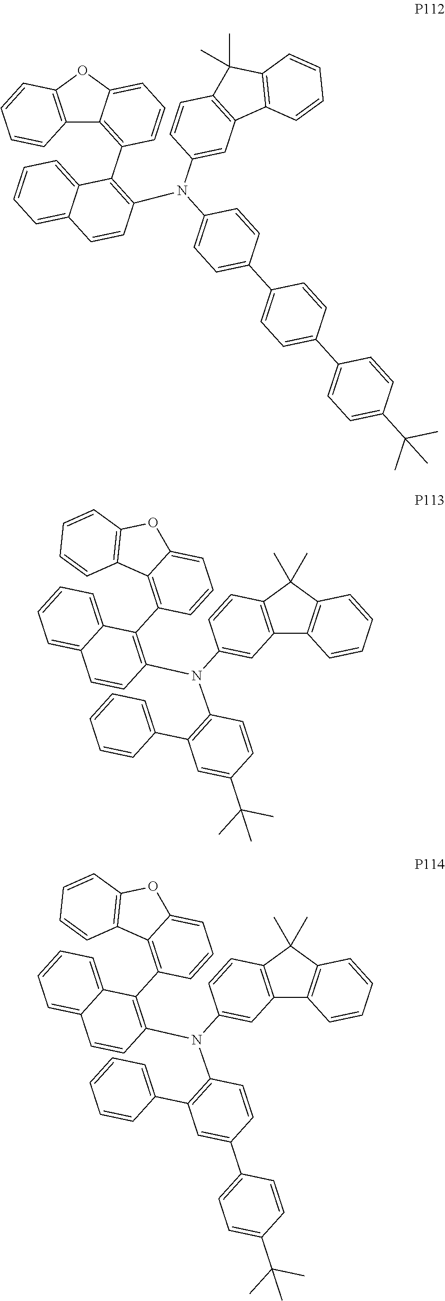 [SMILES: CC(C)(C)c1ccc(-c2ccc(-c3ccc(N(c4ccc5c(c4)-c4ccccc4C5(C)C)c4ccc5ccccc5c4-c4cccc5oc6ccccc6c45)cc3)cc2)cc1 ; CC(C)(C)c1ccc(N(c2ccc3c(c2)-c2ccccc2C3(C)C)c2ccc3ccccc3c2-c2cccc3oc4ccccc4c23)c(-c2ccccc2)c1 ; CC(C)(C)c1ccc(-c2ccc(N(c3ccc4c(c3)-c3ccccc3C4(C)C)c3ccc4ccccc4c3-c3cccc4oc5ccccc5c34)c(-c3ccccc3)c2)cc1]

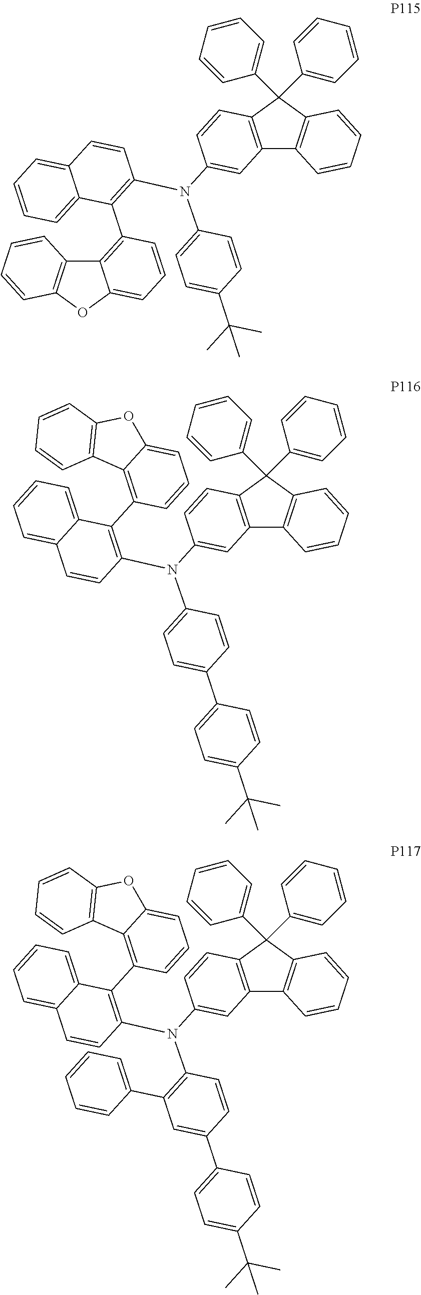

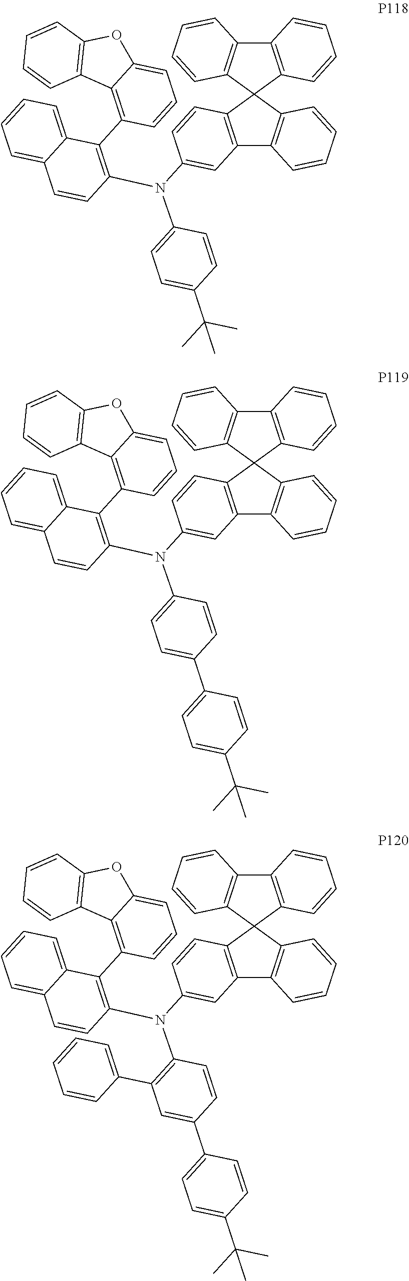

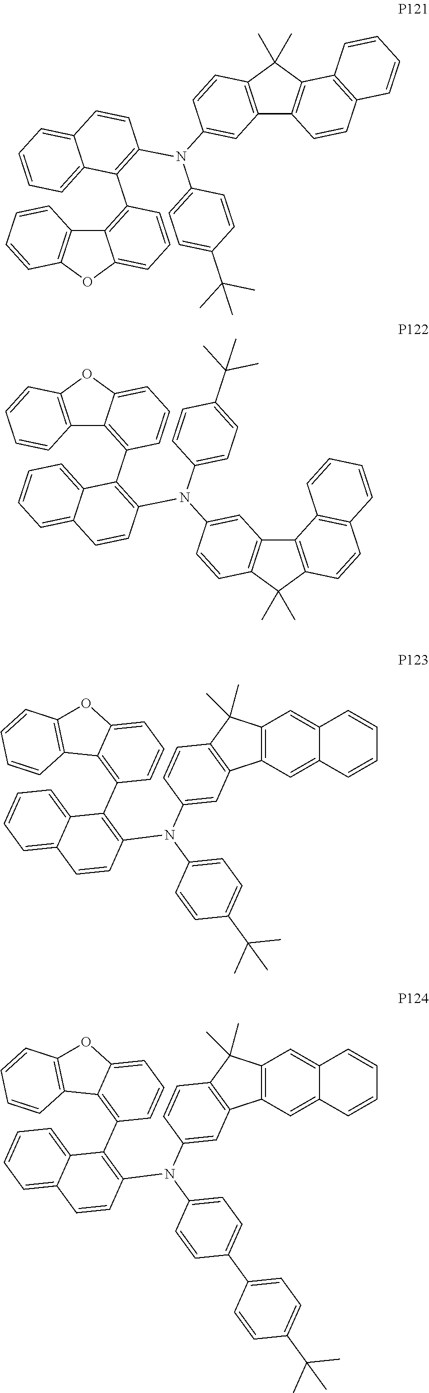

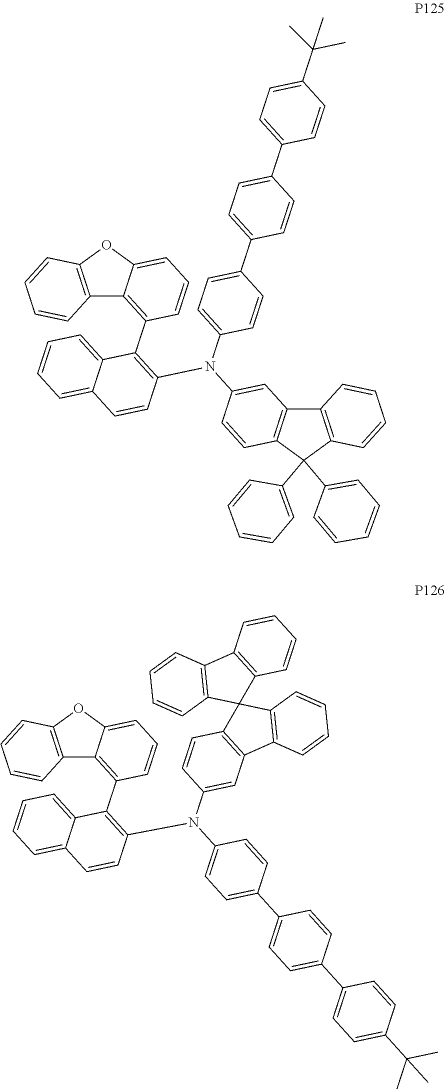 [SMILES: CC(C)(C)c1ccc(-c2ccc(-c3ccc(N(c4ccc5c(c4)-c4ccccc4C5(c4ccccc4)c4ccccc4)c4ccc5ccccc5c4-c4cccc5oc6ccccc6c45)cc3)cc2)cc1 ; CC(C)(C)c1ccc(-c2ccc(-c3ccc(N(c4ccc5c(c4)-c4ccccc4C54c5ccccc5-c5ccccc54)c4ccc5ccccc5c4-c4cccc5oc6ccccc6c45)cc3)cc2)cc1]

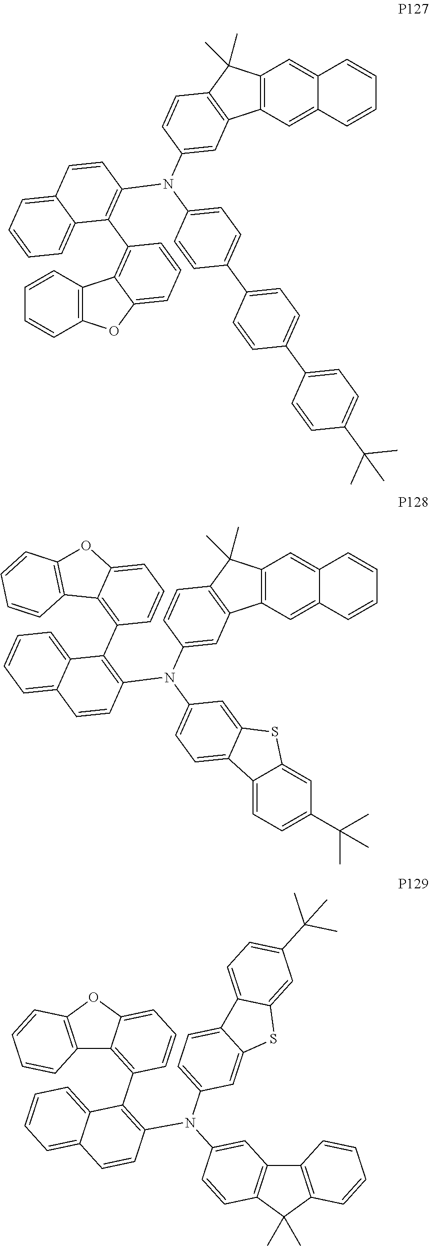

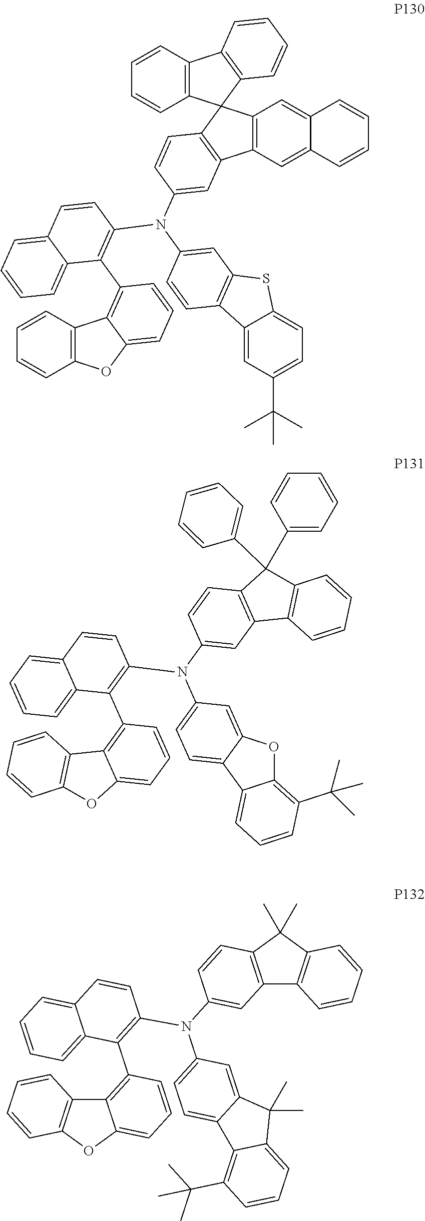

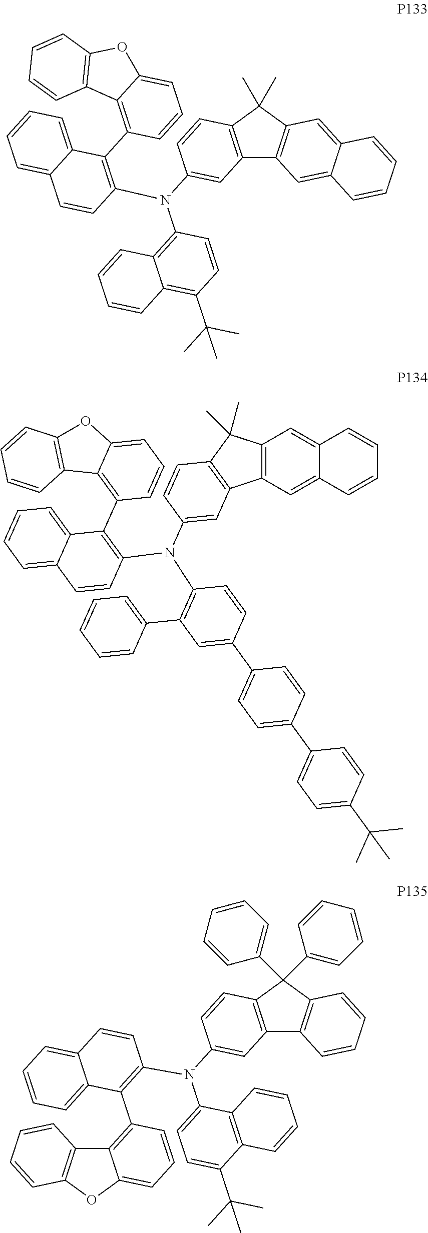 [SMILES: CC(C)(C)c1ccc(N(c2ccc3c(c2)-c2cc4ccccc4cc2C3(C)C)c2ccc3ccccc3c2-c2cccc3oc4ccccc4c23)c2ccccc12 ; CC(C)(C)c1ccc(-c2ccc(-c3ccc(N(c4ccc5c(c4)-c4cc6ccccc6cc4C5(C)C)c4ccc5ccccc5c4-c4cccc5oc6ccccc6c45)c(-c4ccccc4)c3)cc2)cc1 ; CC(C)(C)c1ccc(N(c2ccc3c(c2)-c2ccccc2C3(c2ccccc2)c2ccccc2)c2ccc3ccccc3c2-c2cccc3oc4ccccc4c23)c2ccccc12]

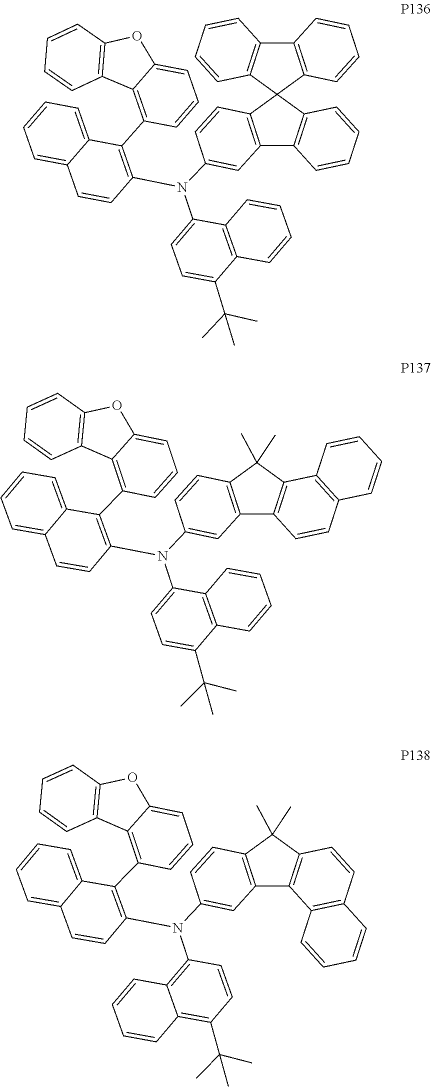 [SMILES: CC(C)(C)c1ccc(N(c2ccc3c(c2)-c2ccccc2C32c3ccccc3-c3ccccc32)c2ccc3ccccc3c2-c2cccc3oc4ccccc4c23)c2ccccc12 ; CC(C)(C)c1ccc(N(c2ccc3c(c2)-c2ccc4ccccc4c2C3(C)C)c2ccc3ccccc3c2-c2cccc3oc4ccccc4c23)c2ccccc12 ; CC(C)(C)c1ccc(N(c2ccc3c(c2)-c2c(ccc4ccccc24)C3(C)C)c2ccc3ccccc3c2-c2cccc3oc4ccccc4c23)c2ccccc12]

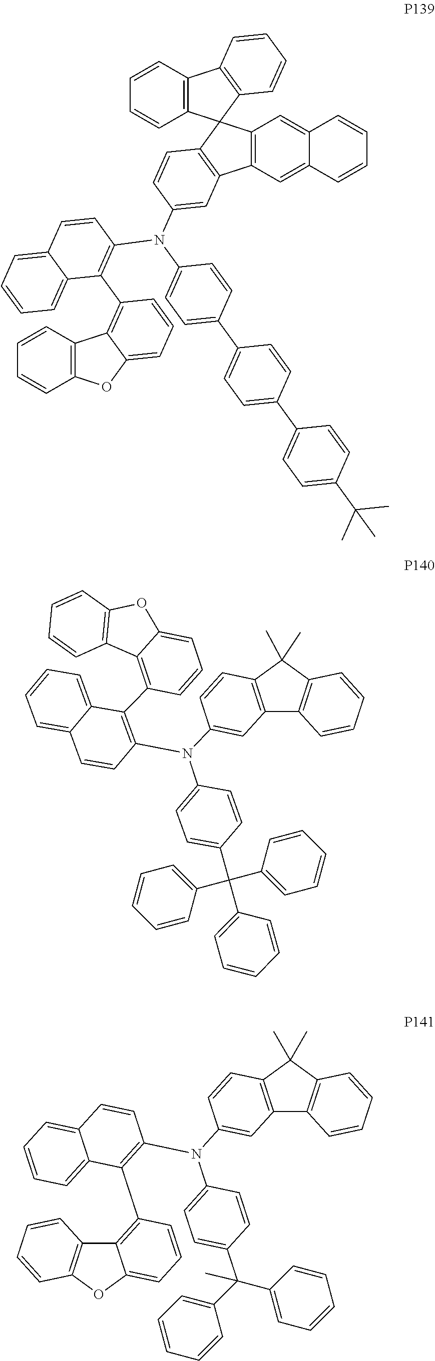 [SMILES: CC(C)(C)c1ccc(-c2ccc(-c3ccc(N(c4ccc5c(c4)-c4cc6ccccc6cc4C54c5ccccc5-c5ccccc54)c4ccc5ccccc5c4-c4cccc5oc6ccccc6c45)cc3)cc2)cc1 ; CC1(C)c2ccccc2-c2cc(N(c3ccc(C(c4ccccc4)(c4ccccc4)c4ccccc4)cc3)c3ccc4ccccc4c3-c3cccc4oc5ccccc5c34)ccc21 ; CC1(C)c2ccccc2-c2cc(N(c3ccc(C(C)(c4ccccc4)c4ccccc4)cc3)c3ccc4ccccc4c3-c3cccc4oc5ccccc5c34)ccc21]

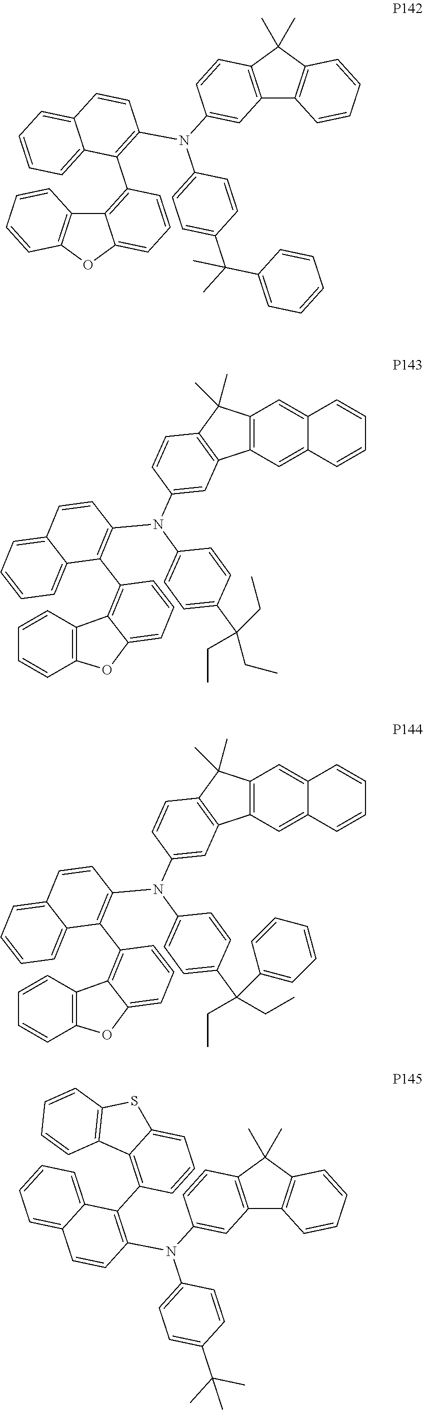 [SMILES: CC(C)(c1ccccc1)c1ccc(N(c2ccc3c(c2)-c2ccccc2C3(C)C)c2ccc3ccccc3c2-c2cccc3oc4ccccc4c23)cc1 ; CCC(CC)(CC)c1ccc(N(c2ccc3c(c2)-c2cc4ccccc4cc2C3(C)C)c2ccc3ccccc3c2-c2cccc3oc4ccccc4c23)cc1 ; CCC(CC)(c1ccccc1)c1ccc(N(c2ccc3c(c2)-c2cc4ccccc4cc2C3(C)C)c2ccc3ccccc3c2-c2cccc3oc4ccccc4c23)cc1 ; CC(C)(C)c1ccc(N(c2ccc3c(c2)-c2ccccc2C3(C)C)c2ccc3ccccc3c2-c2cccc3sc4ccccc4c23)cc1]

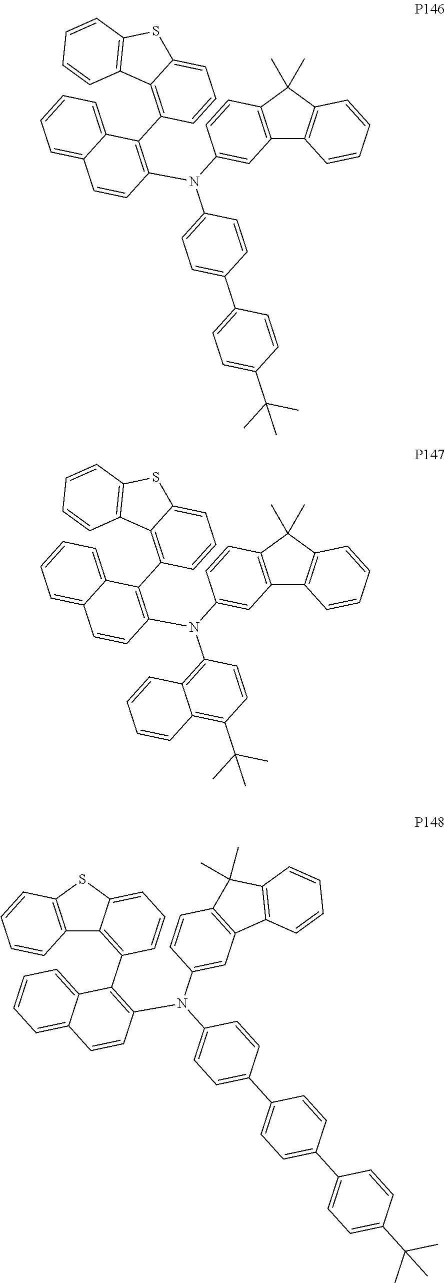 [SMILES: CC(C)(C)c1ccc(-c2ccc(N(c3ccc4c(c3)-c3ccccc3C4(C)C)c3ccc4ccccc4c3-c3cccc4sc5ccccc5c34)cc2)cc1 ; CC(C)(C)c1ccc(N(c2ccc3c(c2)-c2ccccc2C3(C)C)c2ccc3ccccc3c2-c2cccc3sc4ccccc4c23)c2ccccc12 ; CC(C)(C)c1ccc(-c2ccc(-c3ccc(N(c4ccc5c(c4)-c4ccccc4C5(C)C)c4ccc5ccccc5c4-c4cccc5sc6ccccc6c45)cc3)cc2)cc1]

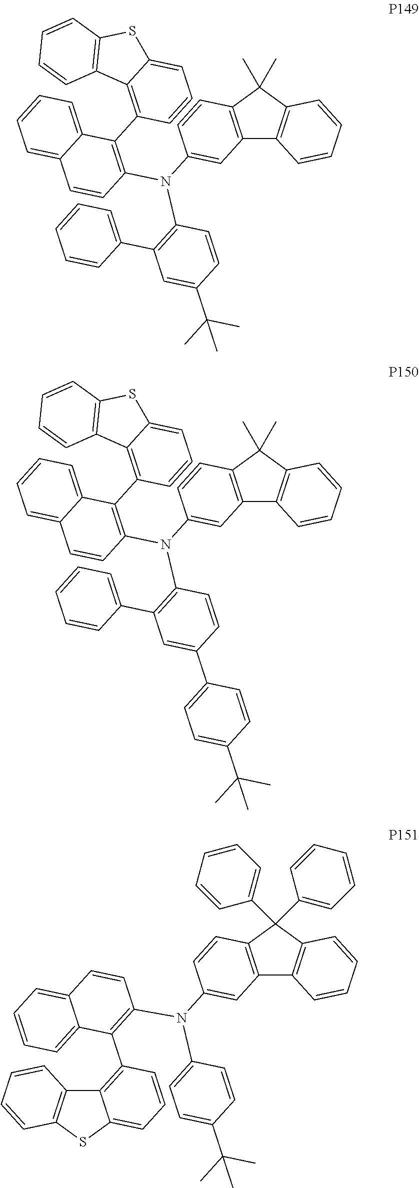

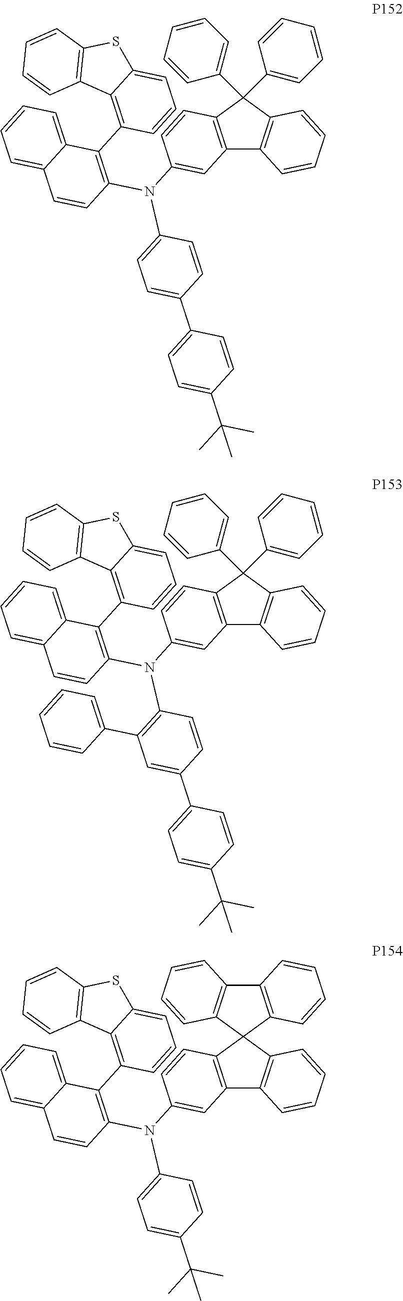

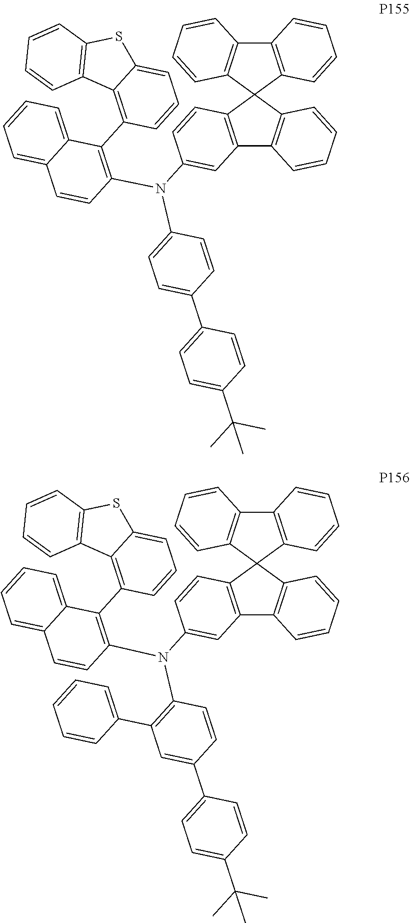

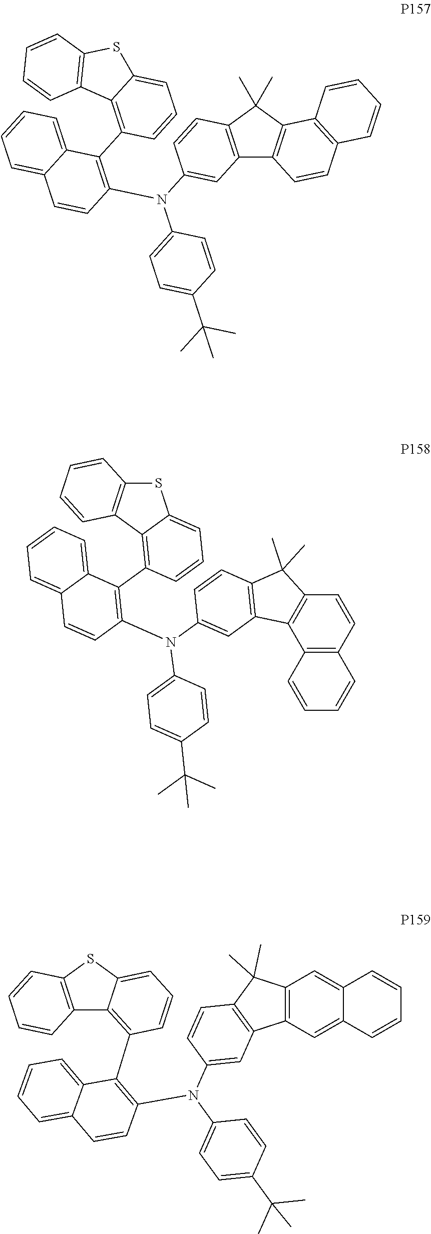 [SMILES: CC(C)(C)c1ccc(N(c2ccc3c(c2)-c2ccc4ccccc4c2C3(C)C)c2ccc3ccccc3c2-c2cccc3sc4ccccc4c23)cc1 ; CC(C)(C)c1ccc(N(c2ccc3c(c2)-c2c(ccc4ccccc24)C3(C)C)c2ccc3ccccc3c2-c2cccc3sc4ccccc4c23)cc1 ; CC(C)(C)c1ccc(N(c2ccc3c(c2)-c2cc4ccccc4cc2C3(C)C)c2ccc3ccccc3c2-c2cccc3sc4ccccc4c23)cc1]

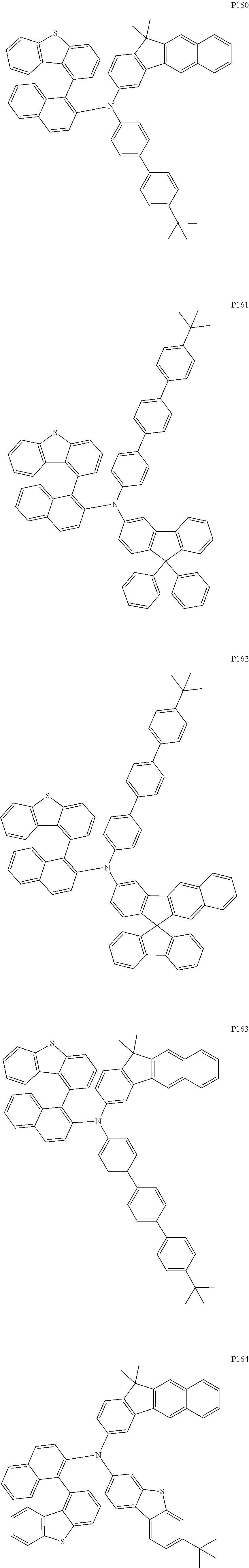

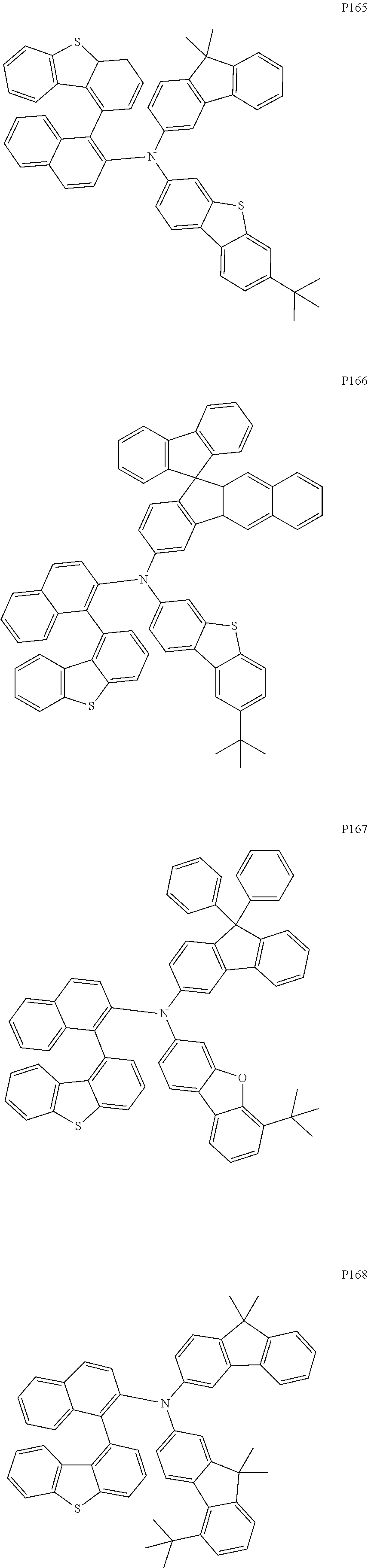 [SMILES: CC(C)(C)c1ccc2c(c1)sc1cc(N(c3ccc4c(c3)-c3ccccc3C4(C)C)c3ccc4ccccc4c3C3=C4c5ccccc5SC4CC=C3)ccc12 ; CC(C)(C)c1ccc2sc3cc(N(c4ccc5c(c4)C4C=c6ccccc6=CC4C54c5ccccc5-c5ccccc54)c4ccc5ccccc5c4-c4cccc5sc6ccccc6c45)ccc3c2c1 ; CC(C)(C)c1cccc2c1oc1cc(N(c3ccc4c(c3)-c3ccccc3C4(c3ccccc3)c3ccccc3)c3ccc4ccccc4c3-c3cccc4sc5ccccc5c34)ccc12 ; CC(C)(C)c1cccc2c1-c1ccc(N(c3ccc4c(c3)-c3ccccc3C4(C)C)c3ccc4ccccc4c3-c3cccc4sc5ccccc5c34)cc1C2(C)C]

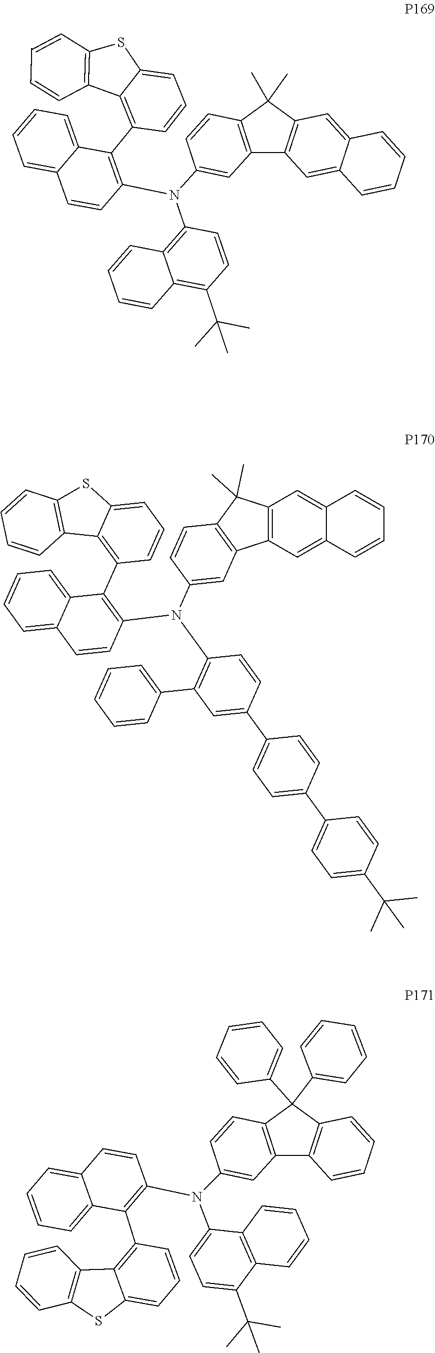 [SMILES: Cc1ccc2c(c1)sc1cccc(-c3c(N(c4ccc5c(c4)-c4cc6ccccc6cc4C5(C)C)c4ccc(C(C)(C)C)c5ccccc45)ccc4ccccc34)c12 ; CC(C)(C)c1ccc(-c2ccc(-c3ccc(N(c4ccc5c(c4)-c4cc6ccccc6cc4C5(C)C)c4ccc5ccccc5c4-c4cccc5sc6ccccc6c45)c(-c4ccccc4)c3)cc2)cc1 ; CC(C)(C)c1ccc(N(c2ccc3c(c2)-c2ccccc2C3(c2ccccc2)c2ccccc2)c2ccc3ccccc3c2-c2cccc3sc4ccccc4c23)c2ccccc12]

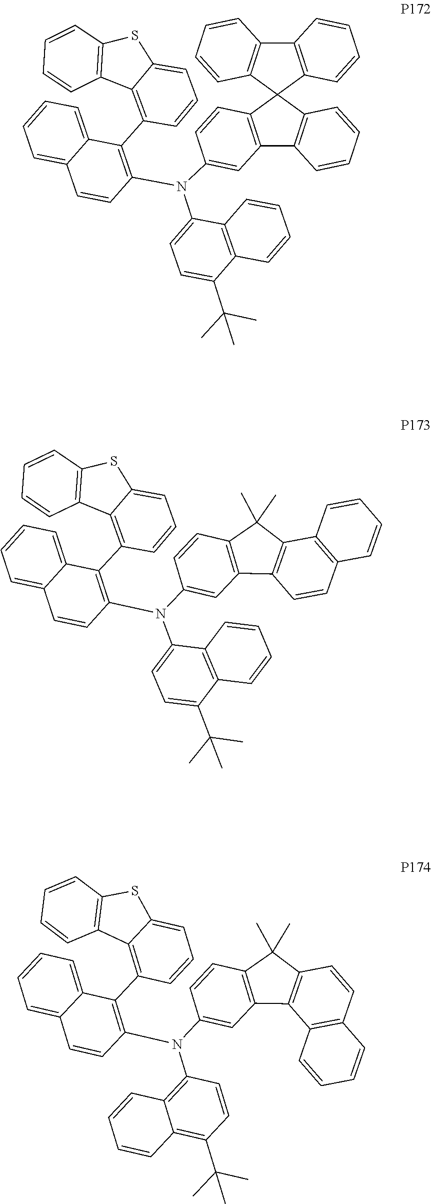 [SMILES: CC(C)(C)c1ccc(N(c2ccc3c(c2)-c2ccccc2C32c3ccccc3-c3ccccc32)c2ccc3ccccc3c2-c2cccc3sc4ccccc4c23)c2ccccc12 ; CC(C)(C)c1ccc(N(c2ccc3c(c2)-c2ccc4ccccc4c2C3(C)C)c2ccc3ccccc3c2-c2cccc3sc4ccccc4c23)c2ccccc12 ; CC(C)(C)c1ccc(N(c2ccc3c(c2)-c2c(ccc4ccccc24)C3(C)C)c2ccc3ccccc3c2-c2cccc3sc4ccccc4c23)c2ccccc12]

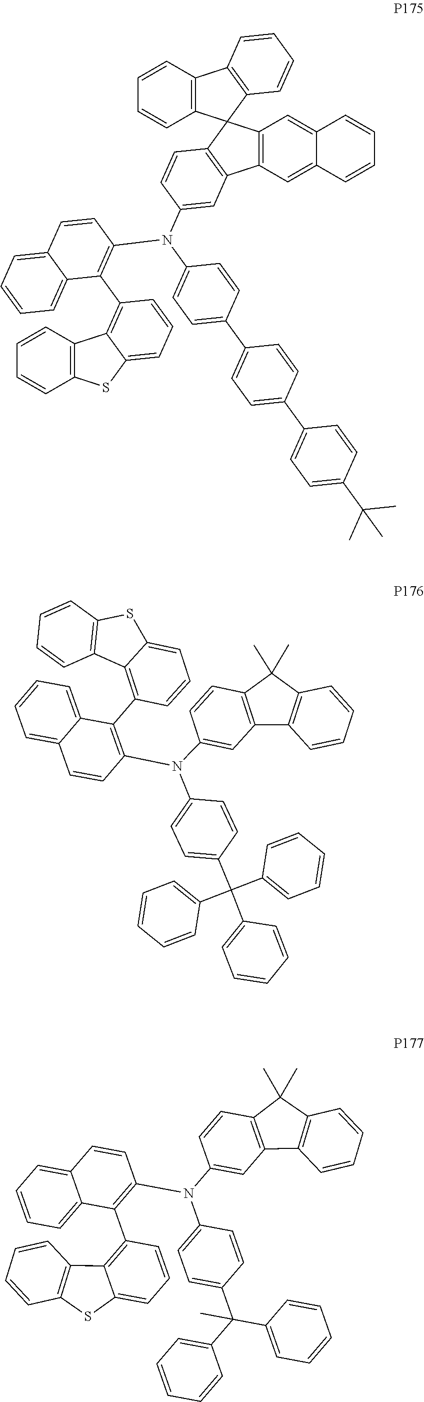 [SMILES: CC(C)(C)c1ccc(-c2ccc(-c3ccc(N(c4ccc5c(c4)-c4cc6ccccc6cc4C54c5ccccc5-c5ccccc54)c4ccc5ccccc5c4-c4cccc5sc6ccccc6c45)cc3)cc2)cc1 ; CC1(C)c2ccccc2-c2cc(N(c3ccc(C(c4ccccc4)(c4ccccc4)c4ccccc4)cc3)c3ccc4ccccc4c3-c3cccc4sc5ccccc5c34)ccc21 ; CC1(C)c2ccccc2-c2cc(N(c3ccc(C(C)(c4ccccc4)c4ccccc4)cc3)c3ccc4ccccc4c3-c3cccc4sc5ccccc5c34)ccc21]

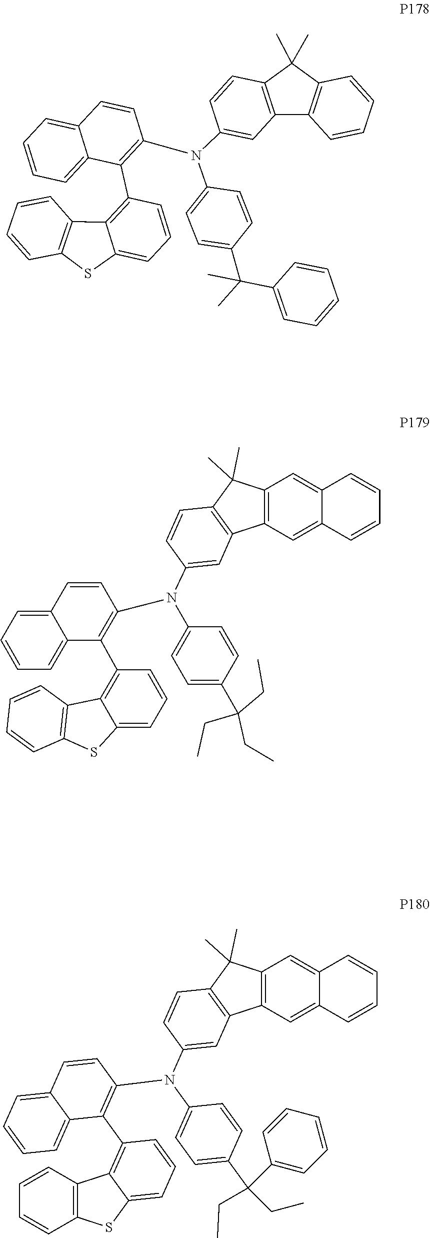 [SMILES: CC(C)(c1ccccc1)c1ccc(N(c2ccc3c(c2)-c2ccccc2C3(C)C)c2ccc3ccccc3c2-c2cccc3sc4ccccc4c23)cc1 ; CCC(CC)(CC)c1ccc(N(c2ccc3c(c2)-c2cc4ccccc4cc2C3(C)C)c2ccc3ccccc3c2-c2cccc3sc4ccccc4c23)cc1 ; CCC(CC)(c1ccccc1)c1ccc(N(c2ccc3c(c2)-c2cc4ccccc4cc2C3(C)C)c2ccc3ccccc3c2-c2cccc3sc4ccccc4c23)cc1]

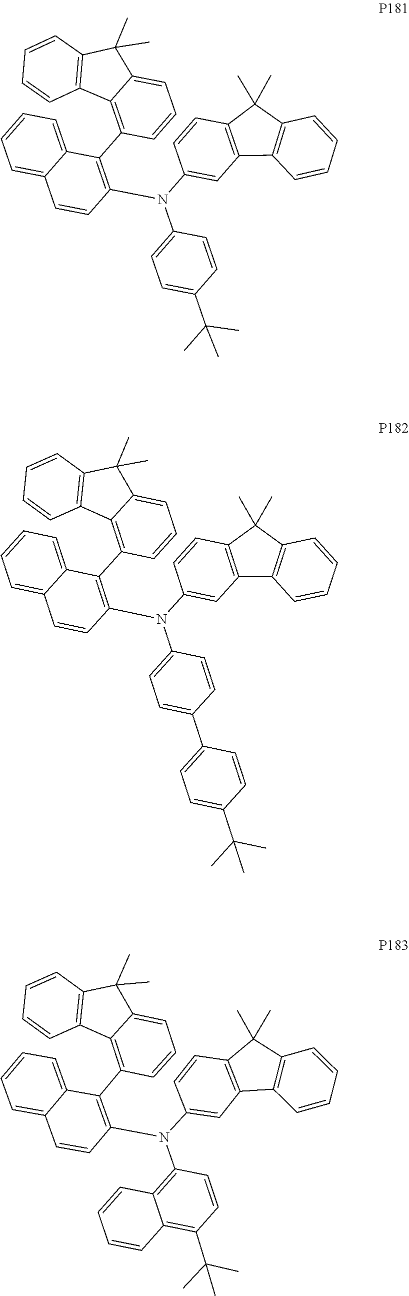 [SMILES: CC(C)(C)c1ccc(N(c2ccc3c(c2)-c2ccccc2C3(C)C)c2ccc3ccccc3c2-c2cccc3c2-c2ccccc2C3(C)C)cc1 ; CC(C)(C)c1ccc(-c2ccc(N(c3ccc4c(c3)-c3ccccc3C4(C)C)c3ccc4ccccc4c3-c3cccc4c3-c3ccccc3C4(C)C)cc2)cc1 ; CC(C)(C)c1ccc(N(c2ccc3c(c2)-c2ccccc2C3(C)C)c2ccc3ccccc3c2-c2cccc3c2-c2ccccc2C3(C)C)c2ccccc12]

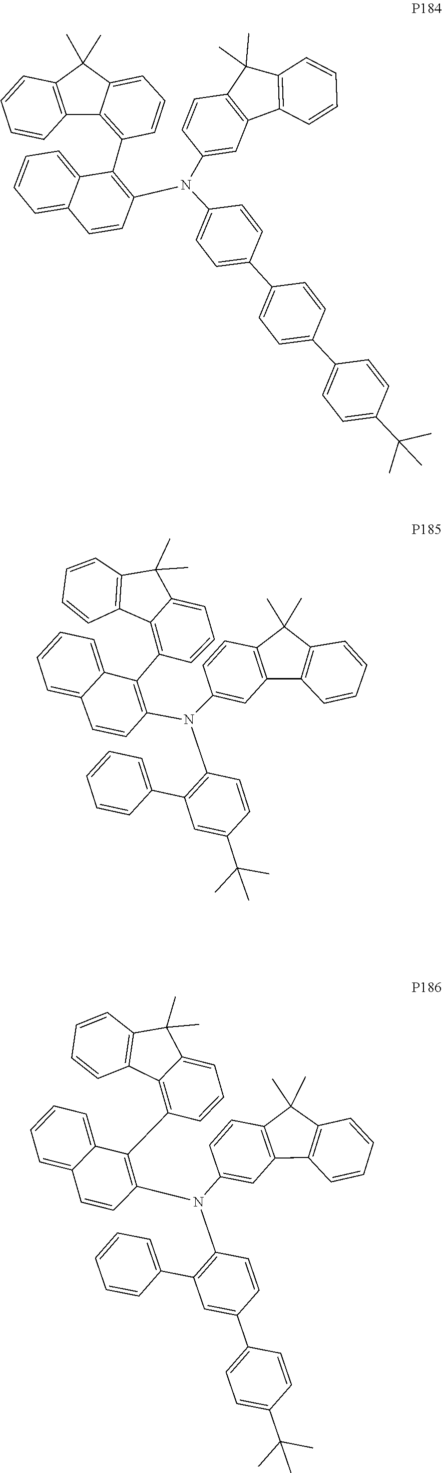 [SMILES: CC(C)(C)c1ccc(-c2ccc(-c3ccc(N(c4ccc5c(c4)-c4ccccc4C5(C)C)c4ccc5ccccc5c4-c4cccc5c4-c4ccccc4C5(C)C)cc3)cc2)cc1 ; CC(C)(C)c1ccc(N(c2ccc3c(c2)-c2ccccc2C3(C)C)c2ccc3ccccc3c2-c2cccc3c2-c2ccccc2C3(C)C)c(-c2ccccc2)c1 ; CC(C)(C)c1ccc(-c2ccc(N(c3ccc4c(c3)-c3ccccc3C4(C)C)c3ccc4ccccc4c3-c3cccc4c3-c3ccccc3C4(C)C)c(-c3ccccc3)c2)cc1]

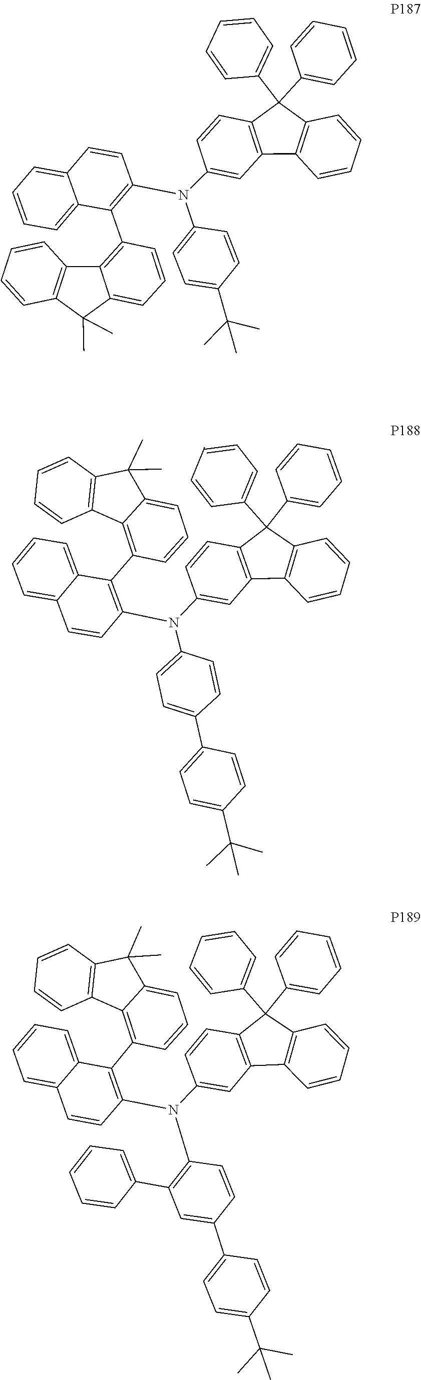 [SMILES: CC(C)(C)c1ccc(N(c2ccc3c(c2)-c2ccccc2C3(c2ccccc2)c2ccccc2)c2ccc3ccccc3c2-c2cccc3c2-c2ccccc2C3(C)C)cc1 ; Cc1ccc(C2(c3ccccc3)c3ccccc3-c3cc(N(c4ccc(-c5ccc(C(C)(C)C)cc5)cc4)c4ccc5ccccc5c4-c4cccc5c4-c4ccccc4C5(C)C)ccc32)cc1 ; CC(C)(C)c1ccc(-c2ccc(N(c3ccc4c(c3)-c3ccccc3C4(c3ccccc3)c3ccccc3)c3ccc4ccccc4c3-c3cccc4c3-c3ccccc3C4(C)C)c(-c3ccccc3)c2)cc1]

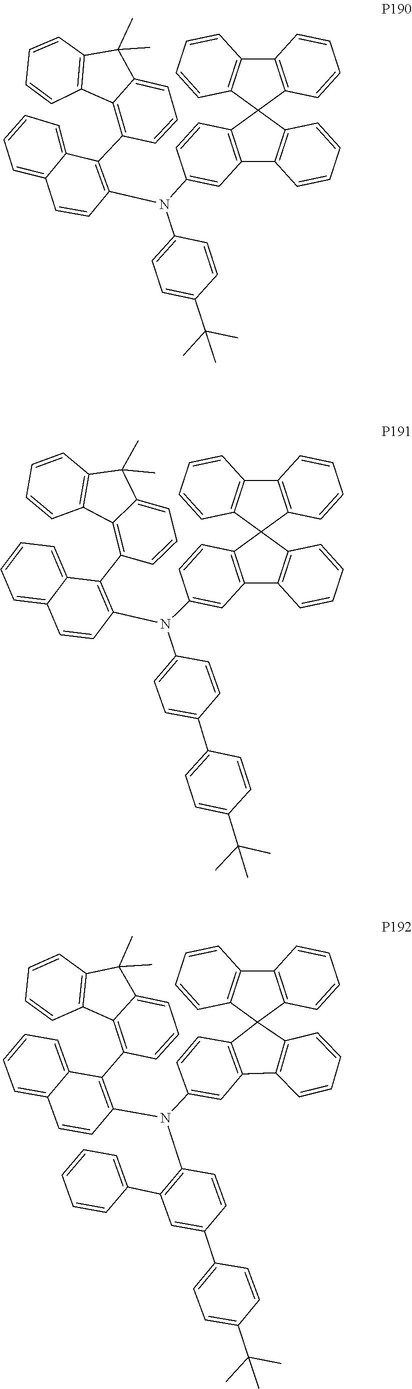 [SMILES: Cc1cccc2ccc(N(c3ccc(C(C)(C)C)cc3)c3ccc4c(c3)-c3ccccc3C43c4ccccc4-c4ccccc43)c(-c3cccc4c3-c3ccccc3C4(C)C)c12 ; CC(C)(C)c1ccc(-c2ccc(N(c3ccc4c(c3)-c3ccccc3C43c4ccccc4-c4ccccc43)c3ccc4ccccc4c3-c3cccc4c3-c3ccccc3C4(C)C)cc2)cc1 ; CC(C)(C)c1ccc(-c2ccc(N(c3ccc4c(c3)-c3ccccc3C43c4ccccc4-c4ccccc43)c3ccc4ccccc4c3-c3cccc4c3-c3ccccc3C4(C)C)c(-c3ccccc3)c2)cc1]

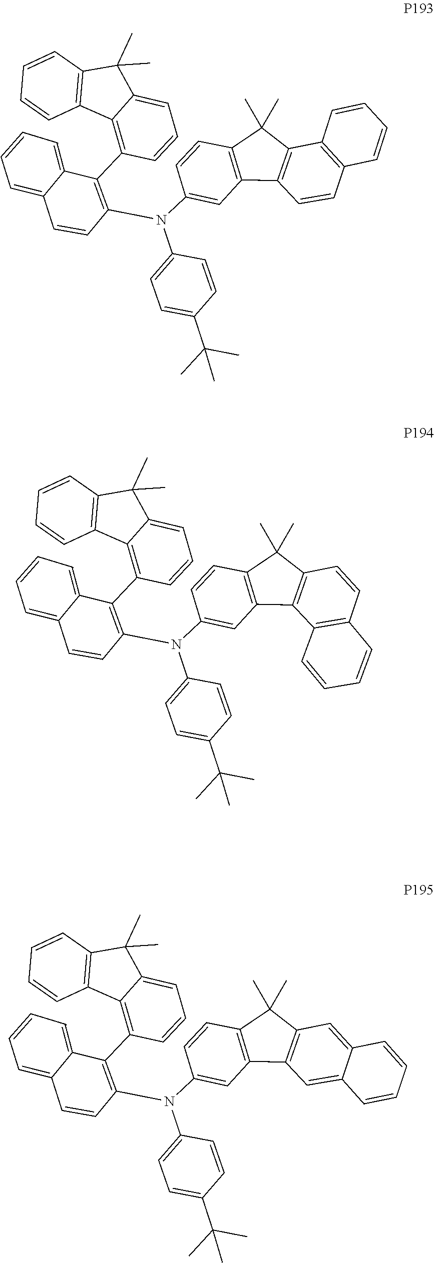 [SMILES: Cc1cccc2ccc(N(c3ccc(C(C)(C)C)cc3)c3ccc4c(c3)-c3ccc5ccccc5c3C4(C)C)c(-c3cccc4c3-c3ccccc3C4(C)C)c12 ; Cc1cccc2ccc(N(c3ccc(C(C)(C)C)cc3)c3ccc4c(c3)-c3c(ccc5ccccc35)C4(C)C)c(-c3cccc4c3-c3ccccc3C4(C)C)c12 ; Cc1cccc2ccc(N(c3ccc(C(C)(C)C)cc3)c3ccc4c(c3)-c3cc5ccccc5cc3C4(C)C)c(-c3cccc4c3-c3ccccc3C4(C)C)c12]

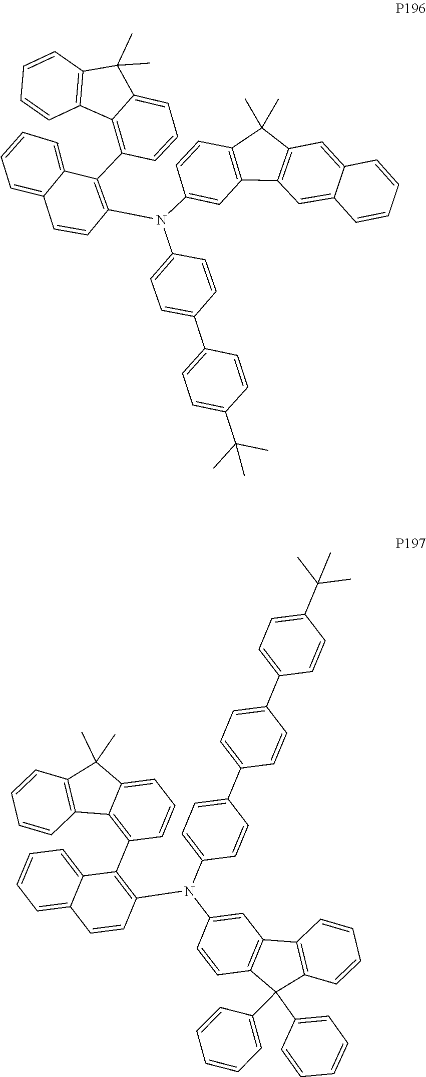 [SMILES: Cc1cccc2ccc(N(c3ccc(-c4ccc(C(C)(C)C)cc4)cc3)c3ccc4c(c3)-c3cc5ccccc5cc3C4(C)C)c(-c3cccc4c3-c3ccccc3C4(C)C)c12 ; CC(C)(C)c1ccc(-c2ccc(-c3ccc(N(c4ccc5c(c4)-c4ccccc4C5(c4ccccc4)c4ccccc4)c4ccc5ccccc5c4-c4cccc5c4-c4ccccc4C5(C)C)cc3)cc2)cc1]

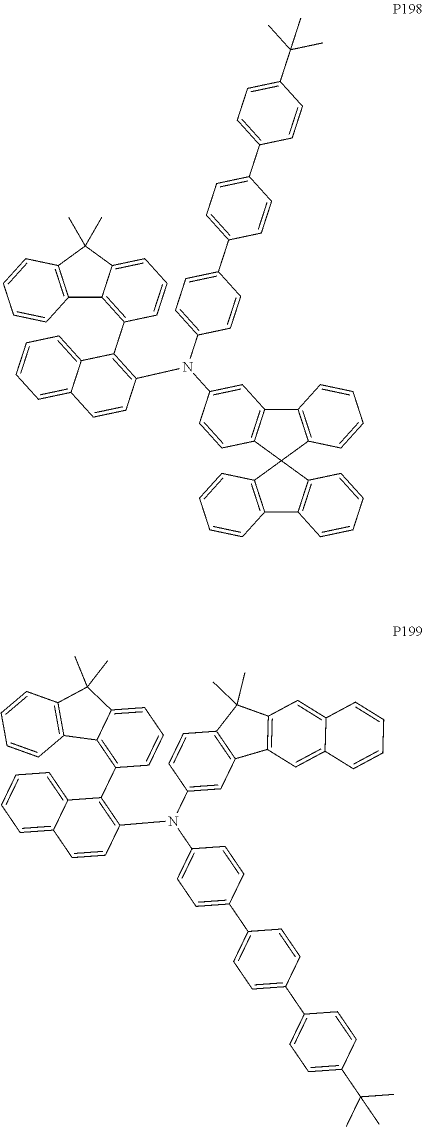 [SMILES: CC(C)(C)c1ccc(-c2ccc(-c3ccc(N(c4ccc5c(c4)-c4ccccc4C54c5ccccc5-c5ccccc54)c4ccc5ccccc5c4-c4cccc5c4-c4ccccc4C5(C)C)cc3)cc2)cc1 ; CC(C)(C)c1ccc(-c2ccc(-c3ccc(N(c4ccc5c(c4)-c4cc6ccccc6cc4C5(C)C)c4ccc5ccccc5c4-c4cccc5c4-c4ccccc4C5(C)C)cc3)cc2)cc1]

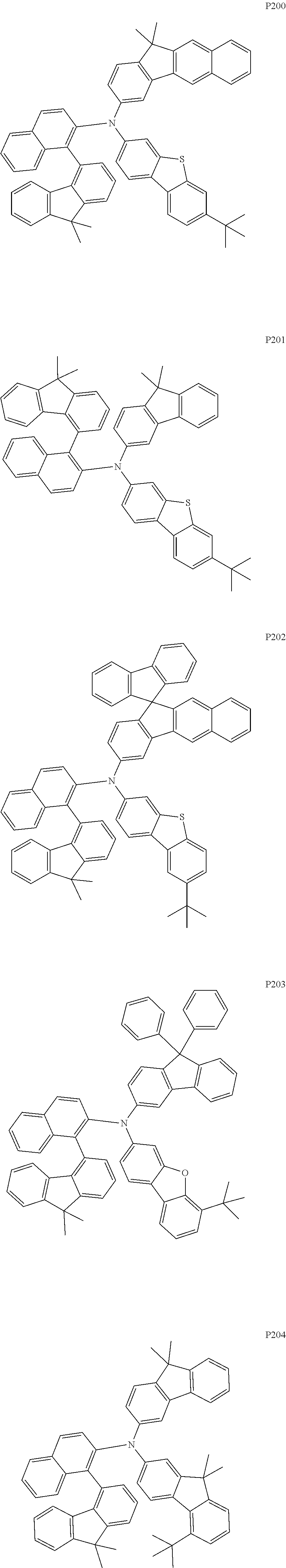

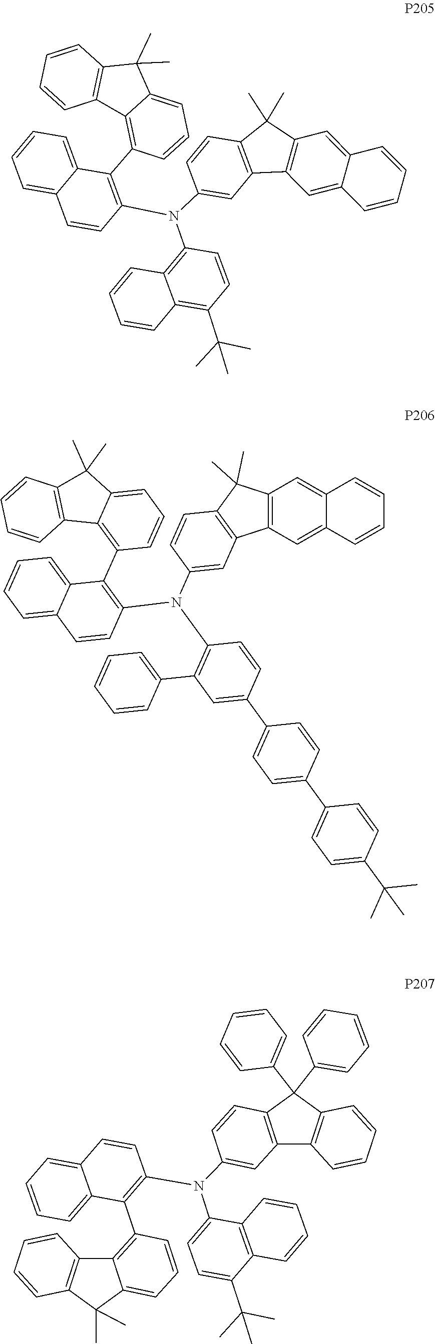 [SMILES: CC(C)(C)c1ccc(N(c2ccc3c(c2)-c2cc4ccccc4cc2C3(C)C)c2ccc3ccccc3c2-c2cccc3c2-c2ccccc2C3(C)C)c2ccccc12 ; CC(C)(C)c1ccc(-c2ccc(-c3ccc(N(c4ccc5c(c4)-c4cc6ccccc6cc4C5(C)C)c4ccc5ccccc5c4-c4cccc5c4-c4ccccc4C5(C)C)c(-c4ccccc4)c3)cc2)cc1 ; CC(C)(C)c1ccc(N(c2ccc3c(c2)-c2ccccc2C3(c2ccccc2)c2ccccc2)c2ccc3ccccc3c2-c2cccc3c2-c2ccccc2C3(C)C)c2ccccc12]

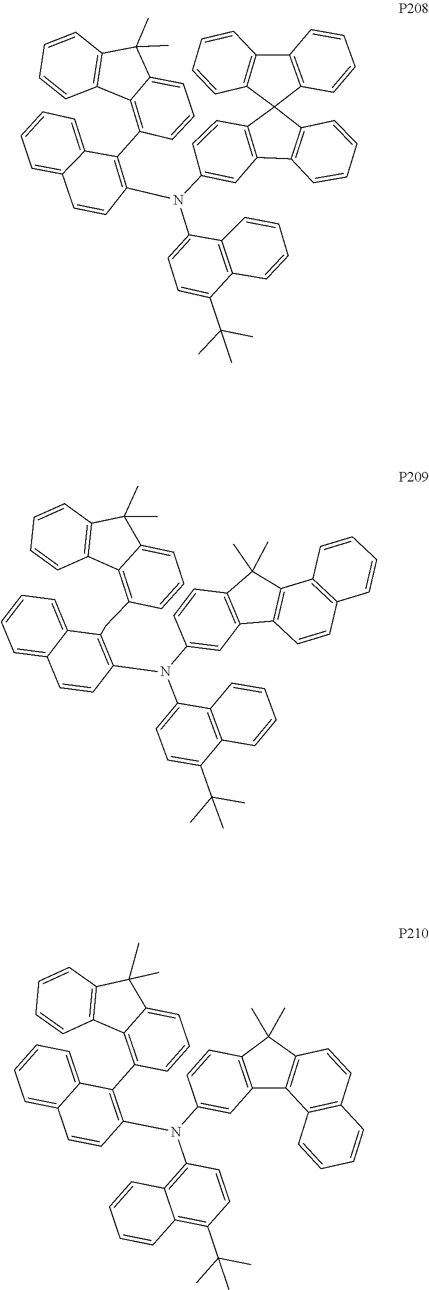 [SMILES: CC(C)(C)c1ccc(N(c2ccc3c(c2)-c2ccccc2C32c3ccccc3-c3ccccc32)c2ccc3ccccc3c2-c2cccc3c2-c2ccccc2C3(C)C)c2ccccc12 ; CC(C)(C)c1ccc(N(c2ccc3c(c2)-c2ccc4ccccc4c2C3(C)C)c2ccc3ccccc3c2Cc2cccc3c2-c2ccccc2C3(C)C)c2ccccc12 ; CC(C)(C)c1ccc(N(c2ccc3c(c2)-c2c(ccc4ccccc24)C3(C)C)c2ccc3ccccc3c2-c2cccc3c2-c2ccccc2C3(C)C)c2ccccc12]

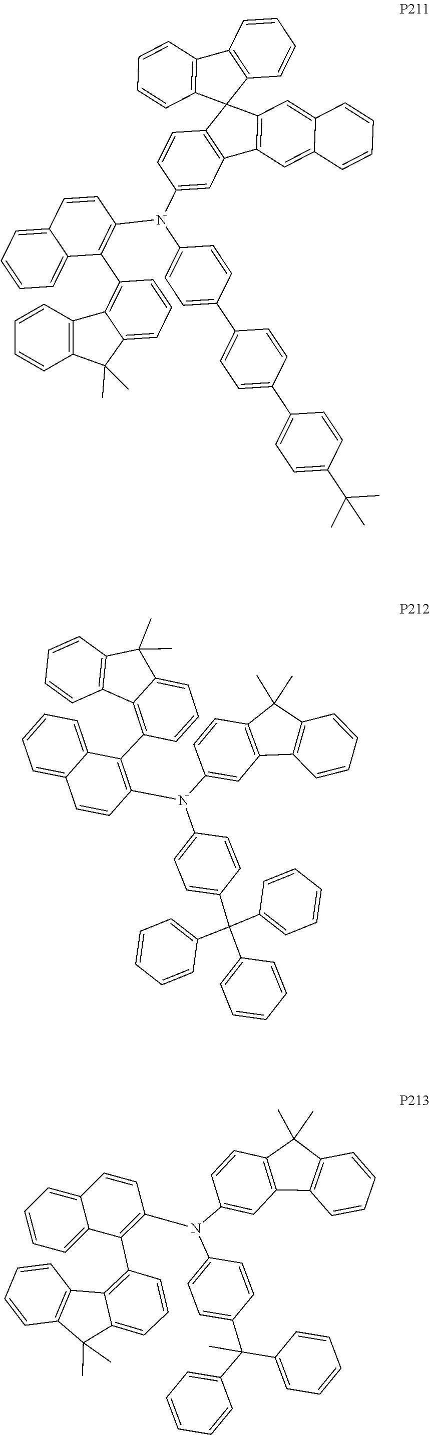 [SMILES: CC(C)(C)c1ccc(-c2ccc(-c3ccc(N(c4ccc5c(c4)-c4cc6ccccc6cc4C54c5ccccc5-c5ccccc54)c4ccc5ccccc5c4-c4cccc5c4-c4ccccc4C5(C)C)cc3)cc2)cc1 ; CC1(C)c2ccccc2-c2cc(N(c3ccc(C(c4ccccc4)(c4ccccc4)c4ccccc4)cc3)c3ccc4ccccc4c3-c3cccc4c3-c3ccccc3C4(C)C)ccc21 ; CC1(C)c2ccccc2-c2cc(N(c3ccc(C(C)(c4ccccc4)c4ccccc4)cc3)c3ccc4ccccc4c3-c3cccc4c3-c3ccccc3C4(C)C)ccc21]

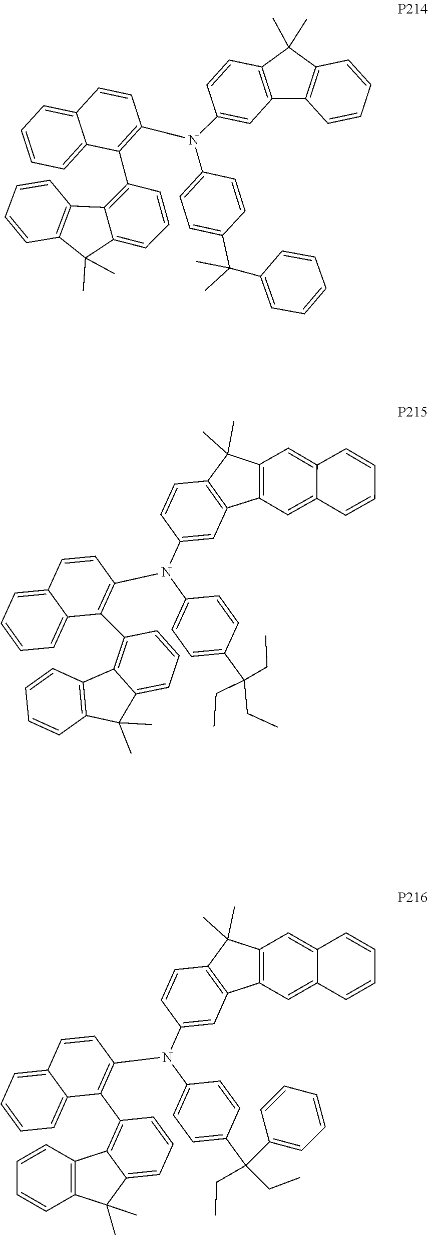 [SMILES: CC(C)(c1ccccc1)c1ccc(N(c2ccc3c(c2)-c2ccccc2C3(C)C)c2ccc3ccccc3c2-c2cccc3c2-c2ccccc2C3(C)C)cc1 ; CCC(CC)(CC)c1ccc(N(c2ccc3c(c2)-c2cc4ccccc4cc2C3(C)C)c2ccc3ccccc3c2-c2cccc3c2-c2ccccc2C3(C)C)cc1 ; CCC(CC)(c1ccccc1)c1ccc(N(c2ccc3c(c2)-c2cc4ccccc4cc2C3(C)C)c2ccc3ccccc3c2-c2cccc3c2-c2ccccc2C3(C)C)cc1]

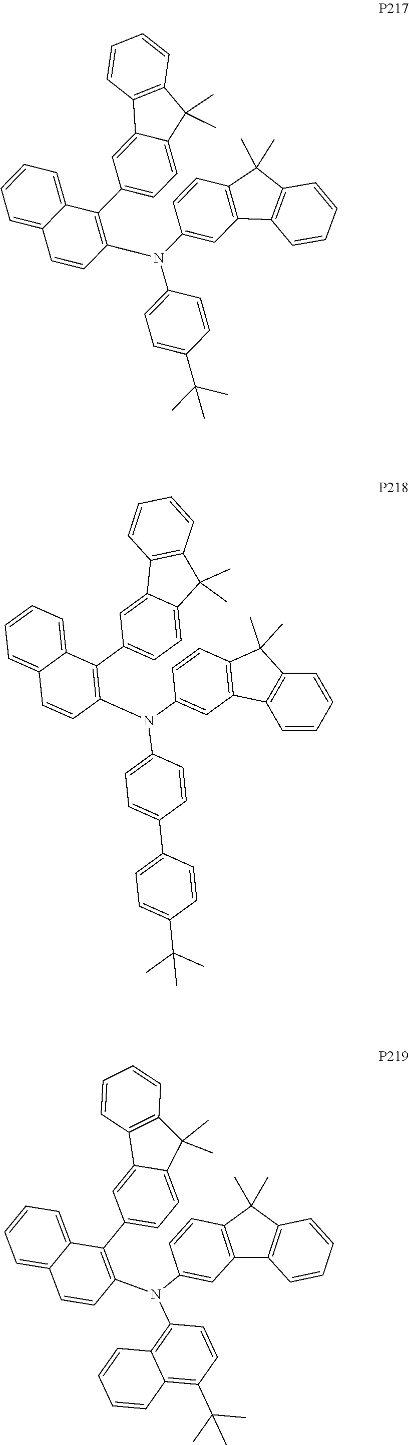 [SMILES: CC(C)(C)c1ccc(N(c2ccc3c(c2)-c2ccccc2C3(C)C)c2ccc3ccccc3c2-c2ccc3c(c2)-c2ccccc2C3(C)C)cc1 ; CC(C)(C)c1ccc(-c2ccc(N(c3ccc4c(c3)-c3ccccc3C4(C)C)c3ccc4ccccc4c3-c3ccc4c(c3)-c3ccccc3C4(C)C)cc2)cc1 ; CC(C)(C)c1ccc(N(c2ccc3c(c2)-c2ccccc2C3(C)C)c2ccc3ccccc3c2-c2ccc3c(c2)-c2ccccc2C3(C)C)c2ccccc12]

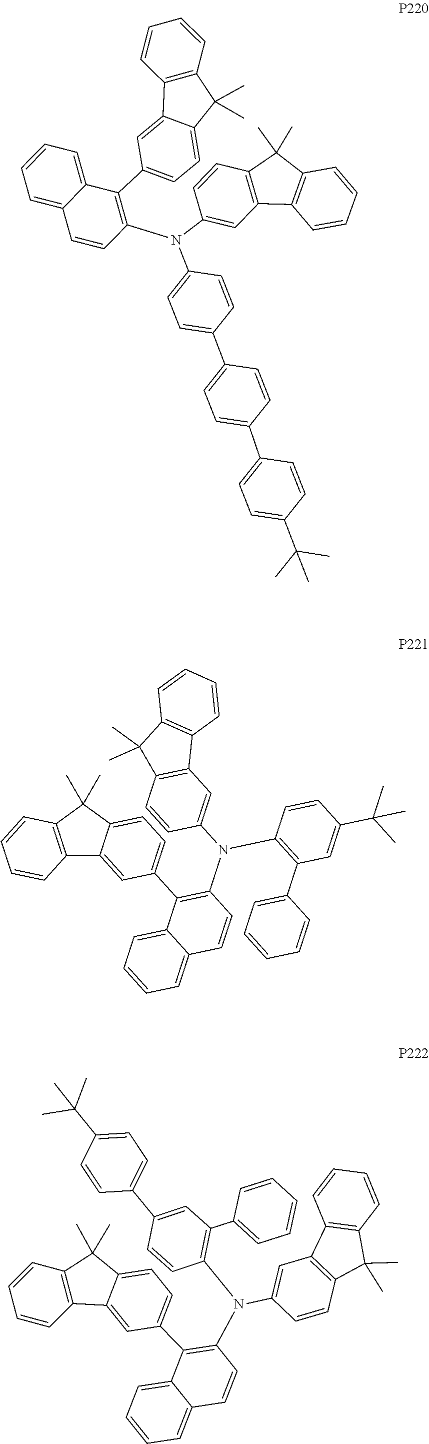 [SMILES: CC(C)(C)c1ccc(-c2ccc(-c3ccc(N(c4ccc5c(c4)-c4ccccc4C5(C)C)c4ccc5ccccc5c4-c4ccc5c(c4)-c4ccccc4C5(C)C)cc3)cc2)cc1 ; CC(C)(C)c1ccc(N(c2ccc3c(c2)-c2ccccc2C3(C)C)c2ccc3ccccc3c2-c2ccc3c(c2)-c2ccccc2C3(C)C)c(-c2ccccc2)c1 ; CC(C)(C)c1ccc(-c2ccc(N(c3ccc4c(c3)-c3ccccc3C4(C)C)c3ccc4ccccc4c3-c3ccc4c(c3)-c3ccccc3C4(C)C)c(-c3ccccc3)c2)cc1]

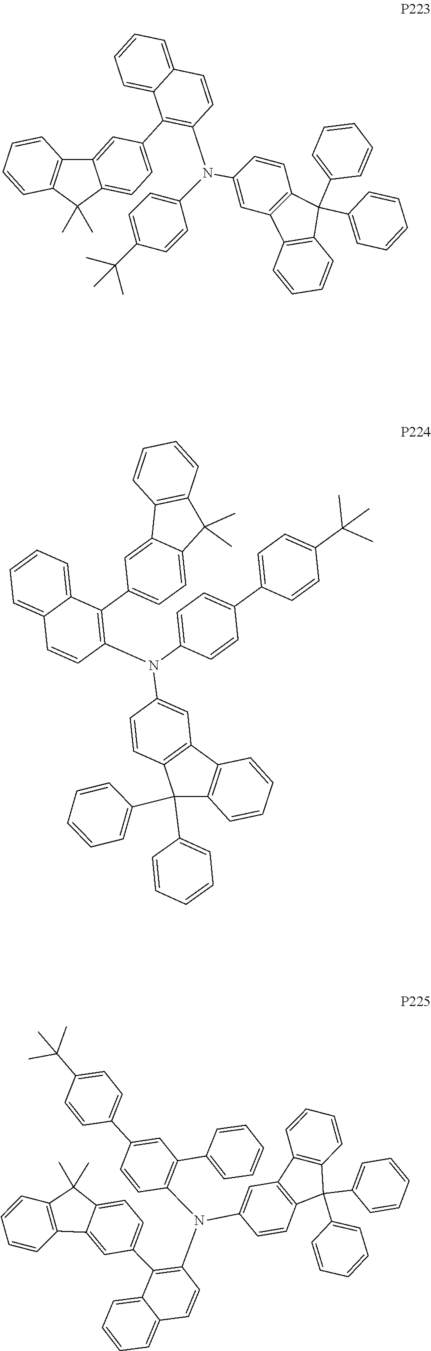 [SMILES: CC(C)(C)c1ccc(N(c2ccc3c(c2)-c2ccccc2C3(c2ccccc2)c2ccccc2)c2ccc3ccccc3c2-c2ccc3c(c2)-c2ccccc2C3(C)C)cc1 ; CC(C)(C)c1ccc(-c2ccc(N(c3ccc4c(c3)-c3ccccc3C4(c3ccccc3)c3ccccc3)c3ccc4ccccc4c3-c3ccc4c(c3)-c3ccccc3C4(C)C)cc2)cc1 ; CC(C)(C)c1ccc(-c2ccc(N(c3ccc4c(c3)-c3ccccc3C4(c3ccccc3)c3ccccc3)c3ccc4ccccc4c3-c3ccc4c(c3)-c3ccccc3C4(C)C)c(-c3ccccc3)c2)cc1]

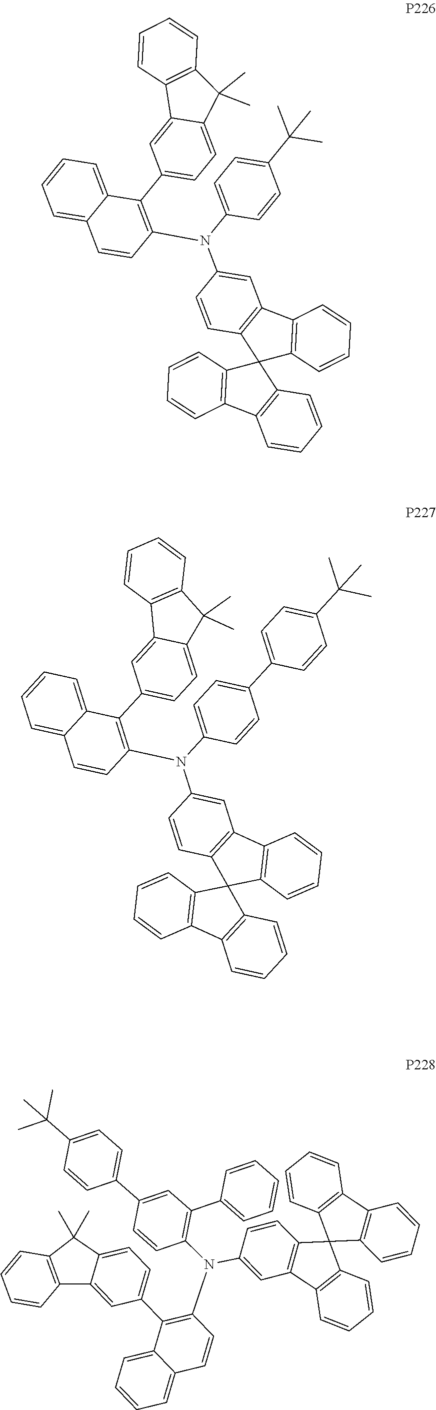 [SMILES: CC(C)(C)c1ccc(N(c2ccc3c(c2)-c2ccccc2C32c3ccccc3-c3ccccc32)c2ccc3ccccc3c2-c2ccc3c(c2)-c2ccccc2C3(C)C)cc1 ; CC(C)(C)c1ccc(-c2ccc(N(c3ccc4c(c3)-c3ccccc3C43c4ccccc4-c4ccccc43)c3ccc4ccccc4c3-c3ccc4c(c3)-c3ccccc3C4(C)C)cc2)cc1 ; CC(C)(C)c1ccc(-c2ccc(N(c3ccc4c(c3)-c3ccccc3C43c4ccccc4-c4ccccc43)c3ccc4ccccc4c3-c3ccc4c(c3)-c3ccccc3C4(C)C)c(-c3ccccc3)c2)cc1]

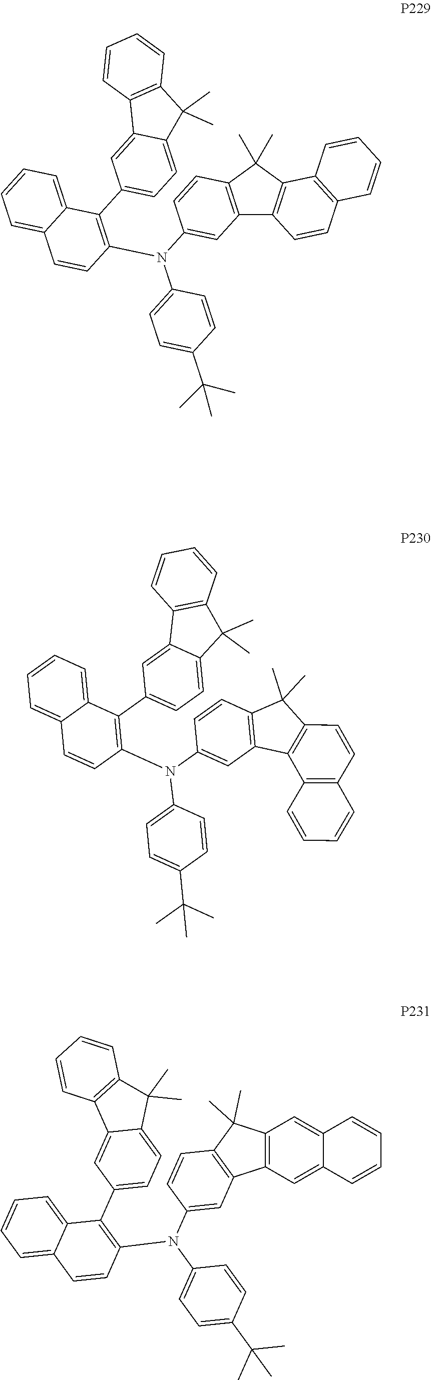 [SMILES: CC(C)(C)c1ccc(N(c2ccc3c(c2)-c2ccc4ccccc4c2C3(C)C)c2ccc3ccccc3c2-c2ccc3c(c2)-c2ccccc2C3(C)C)cc1 ; CC(C)(C)c1ccc(N(c2ccc3c(c2)-c2c(ccc4ccccc24)C3(C)C)c2ccc3ccccc3c2-c2ccc3c(c2)-c2ccccc2C3(C)C)cc1 ; CC(C)(C)c1ccc(N(c2ccc3c(c2)-c2cc4ccccc4cc2C3(C)C)c2ccc3ccccc3c2-c2ccc3c(c2)-c2ccccc2C3(C)C)cc1]

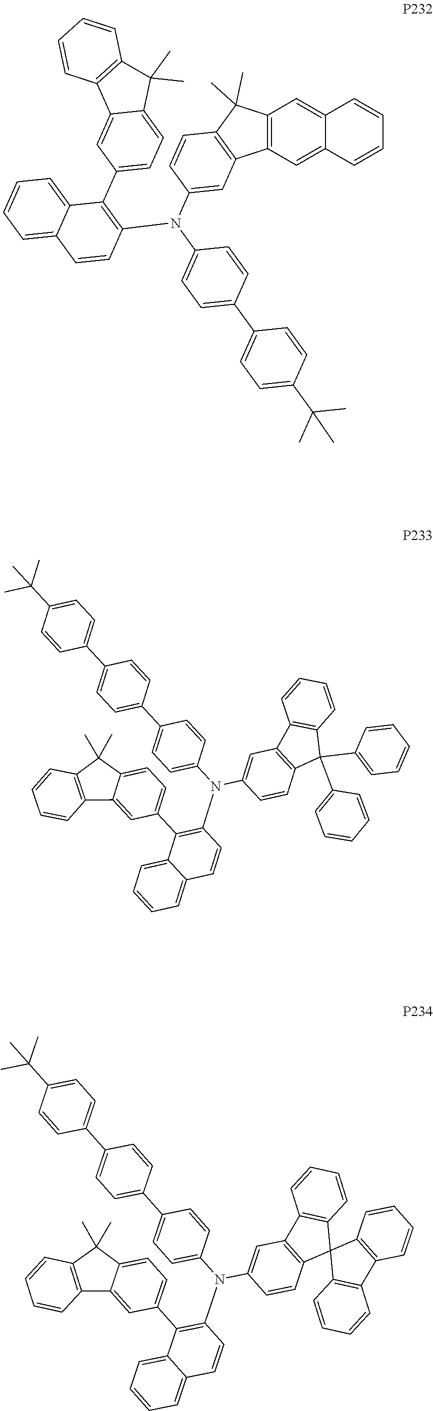 [SMILES: CC(C)(C)c1ccc(-c2ccc(N(c3ccc4c(c3)-c3cc5ccccc5cc3C4(C)C)c3ccc4ccccc4c3-c3ccc4c(c3)-c3ccccc3C4(C)C)cc2)cc1 ; CC(C)(C)c1ccc(-c2ccc(-c3ccc(N(c4ccc5c(c4)-c4ccccc4C5(c4ccccc4)c4ccccc4)c4ccc5ccccc5c4-c4ccc5c(c4)-c4ccccc4C5(C)C)cc3)cc2)cc1 ; CC(C)(C)c1ccc(-c2ccc(-c3ccc(N(c4ccc5c(c4)-c4ccccc4C54c5ccccc5-c5ccccc54)c4ccc5ccccc5c4-c4ccc5c(c4)-c4ccccc4C5(C)C)cc3)cc2)cc1]

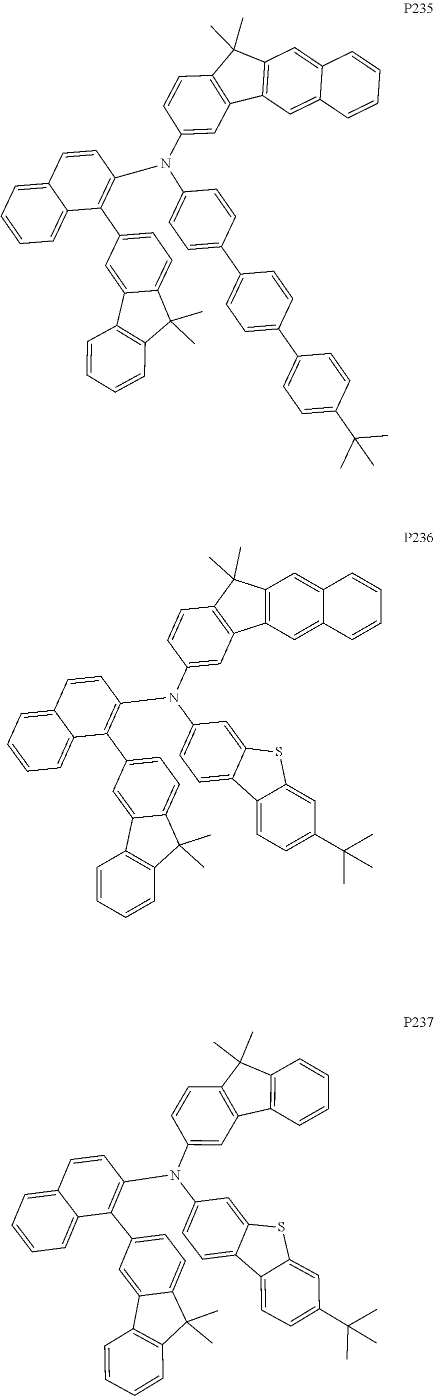

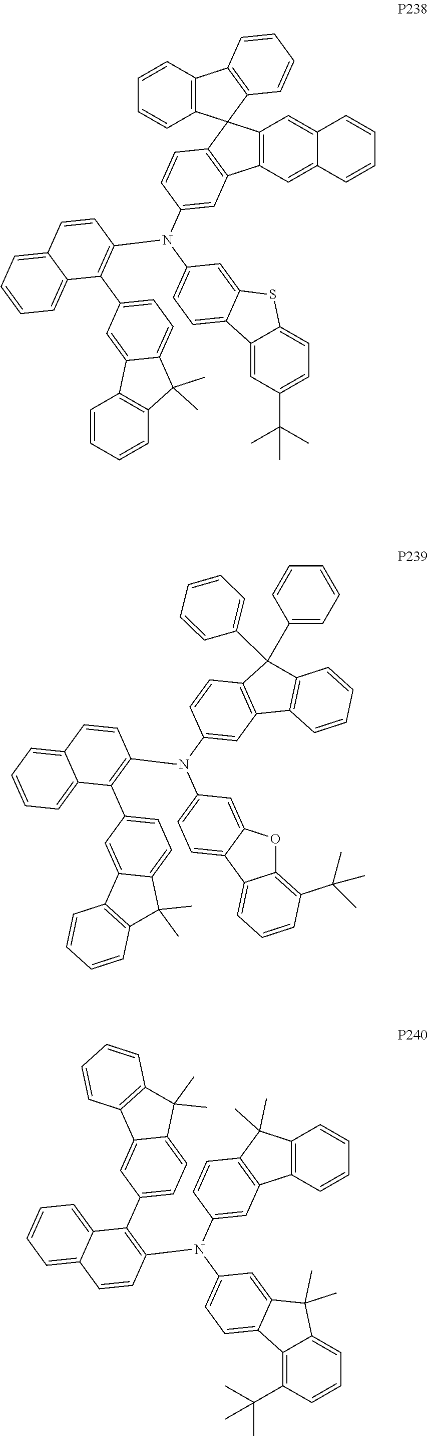 [SMILES: CC(C)(C)c1ccc2sc3cc(N(c4ccc5c(c4)-c4cc6ccccc6cc4C54c5ccccc5-c5ccccc54)c4ccc5ccccc5c4-c4ccc5c(c4)-c4ccccc4C5(C)C)ccc3c2c1 ; CC(C)(C)c1cccc2c1oc1cc(N(c3ccc4c(c3)-c3ccccc3C4(c3ccccc3)c3ccccc3)c3ccc4ccccc4c3-c3ccc4c(c3)-c3ccccc3C4(C)C)ccc12 ; CC(C)(C)c1cccc2c1-c1ccc(N(c3ccc4c(c3)-c3ccccc3C4(C)C)c3ccc4ccccc4c3-c3ccc4c(c3)-c3ccccc3C4(C)C)cc1C2(C)C]

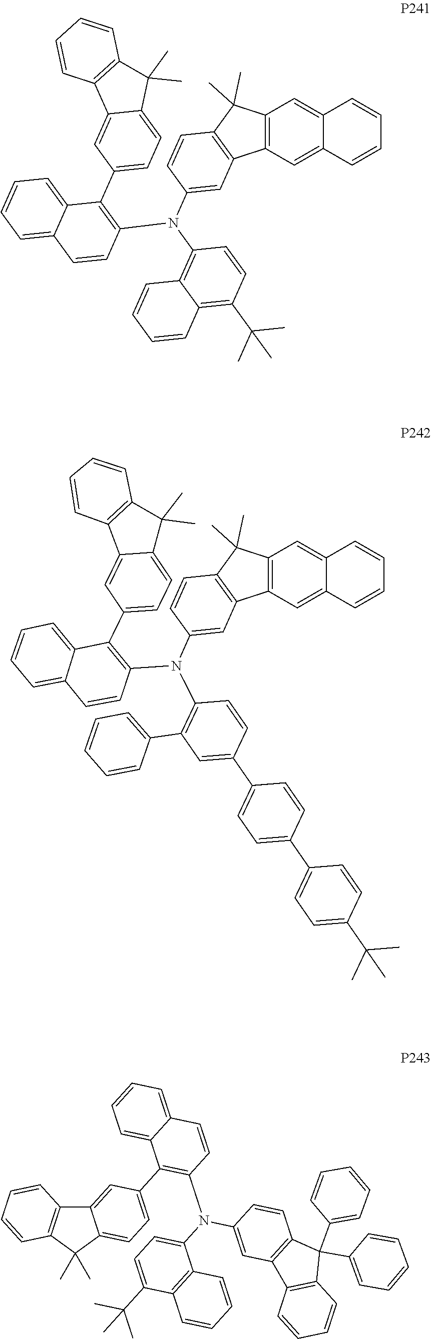 [SMILES: CC(C)(C)c1ccc(N(c2ccc3c(c2)-c2cc4ccccc4cc2C3(C)C)c2ccc3ccccc3c2-c2ccc3c(c2)-c2ccccc2C3(C)C)c2ccccc12 ; CC(C)(C)c1ccc(-c2ccc(-c3ccc(N(c4ccc5c(c4)-c4cc6ccccc6cc4C5(C)C)c4ccc5ccccc5c4-c4ccc5c(c4)-c4ccccc4C5(C)C)c(-c4ccccc4)c3)cc2)cc1 ; CC(C)(C)c1ccc(N(c2ccc3c(c2)-c2ccccc2C3(c2ccccc2)c2ccccc2)c2ccc3ccccc3c2-c2ccc3c(c2)-c2ccccc2C3(C)C)c2ccccc12]

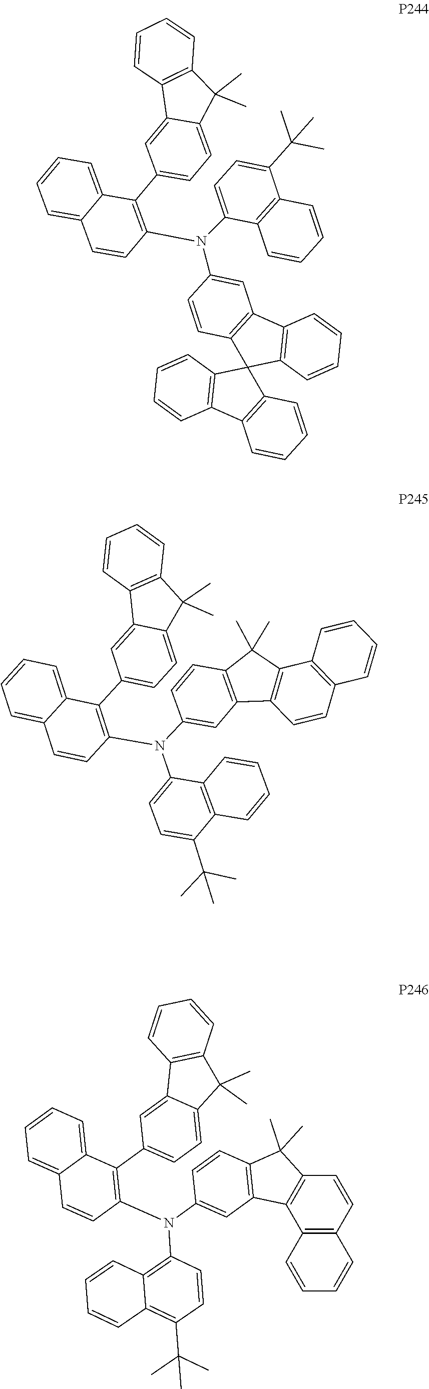 [SMILES: CC(C)(C)c1ccc(N(c2ccc3c(c2)-c2ccccc2C32c3ccccc3-c3ccccc32)c2ccc3ccccc3c2-c2ccc3c(c2)-c2ccccc2C3(C)C)c2ccccc12 ; CC(C)(C)c1ccc(N(c2ccc3c(c2)-c2ccc4ccccc4c2C3(C)C)c2ccc3ccccc3c2-c2ccc3c(c2)-c2ccccc2C3(C)C)c2ccccc12 ; CC(C)(C)c1ccc(N(c2ccc3c(c2)-c2c(ccc4ccccc24)C3(C)C)c2ccc3ccccc3c2-c2ccc3c(c2)-c2ccccc2C3(C)C)c2ccccc12]

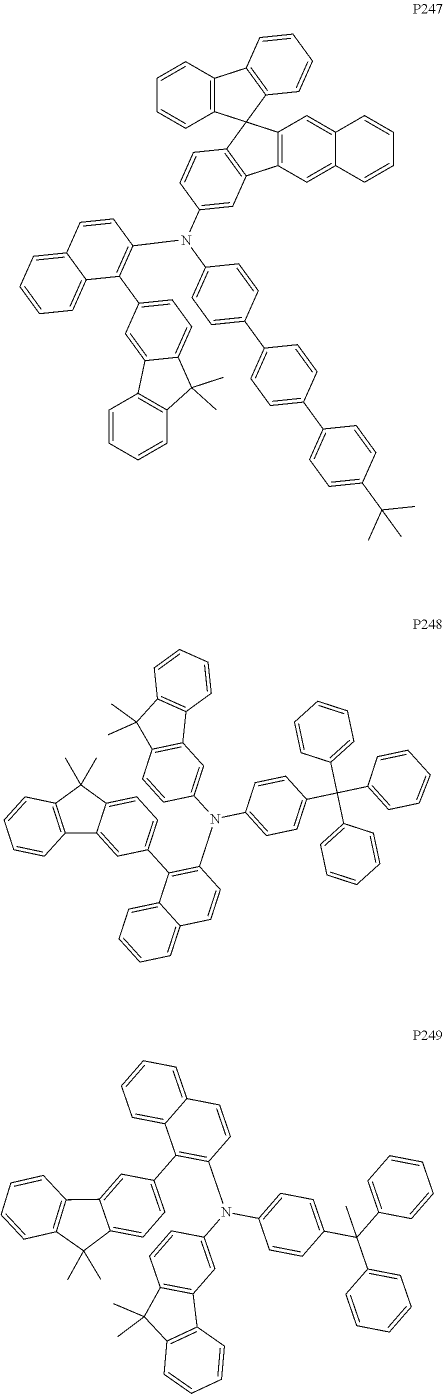 [SMILES: CC(C)(C)c1ccc(-c2ccc(-c3ccc(N(c4ccc5c(c4)-c4cc6ccccc6cc4C54c5ccccc5-c5ccccc54)c4ccc5ccccc5c4-c4ccc5c(c4)-c4ccccc4C5(C)C)cc3)cc2)cc1 ; CC1(C)c2ccccc2-c2cc(-c3c(N(c4ccc(C(c5ccccc5)(c5ccccc5)c5ccccc5)cc4)c4ccc5c(c4)-c4ccccc4C5(C)C)ccc4ccccc34)ccc21 ; CC1(C)c2ccccc2-c2cc(-c3c(N(c4ccc(C(C)(c5ccccc5)c5ccccc5)cc4)c4ccc5c(c4)-c4ccccc4C5(C)C)ccc4ccccc34)ccc21]

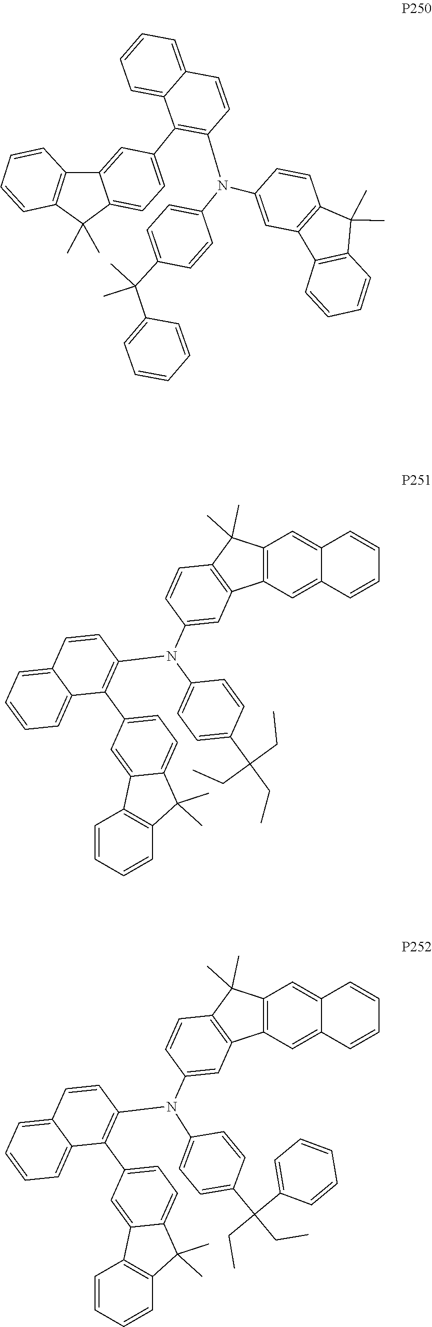 [SMILES: CC(C)(c1ccccc1)c1ccc(N(c2ccc3c(c2)-c2ccccc2C3(C)C)c2ccc3ccccc3c2-c2ccc3c(c2)-c2ccccc2C3(C)C)cc1 ; CCC(CC)(CC)c1ccc(N(c2ccc3c(c2)-c2cc4ccccc4cc2C3(C)C)c2ccc3ccccc3c2-c2ccc3c(c2)-c2ccccc2C3(C)C)cc1 ; CCC(CC)(c1ccccc1)c1ccc(N(c2ccc3c(c2)-c2cc4ccccc4cc2C3(C)C)c2ccc3ccccc3c2-c2ccc3c(c2)-c2ccccc2C3(C)C)cc1]

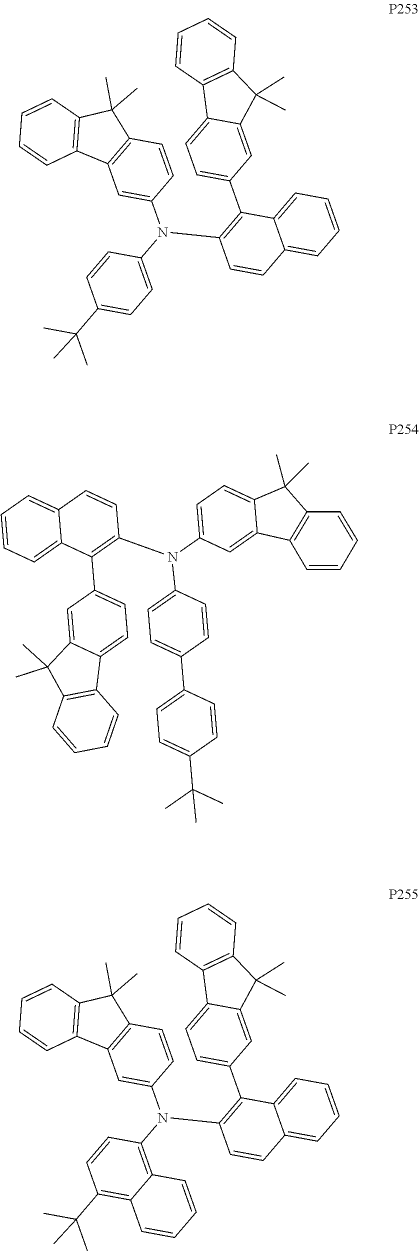 [SMILES: CC(C)(C)c1ccc(N(c2ccc3c(c2)-c2ccccc2C3(C)C)c2ccc3ccccc3c2-c2ccc3c(c2)C(C)(C)c2ccccc2-3)cc1 ; CC(C)(C)c1ccc(-c2ccc(N(c3ccc4c(c3)-c3ccccc3C4(C)C)c3ccc4ccccc4c3-c3ccc4c(c3)C(C)(C)c3ccccc3-4)cc2)cc1 ; CC(C)(C)c1ccc(N(c2ccc3c(c2)-c2ccccc2C3(C)C)c2ccc3ccccc3c2-c2ccc3c(c2)C(C)(C)c2ccccc2-3)c2ccccc12]

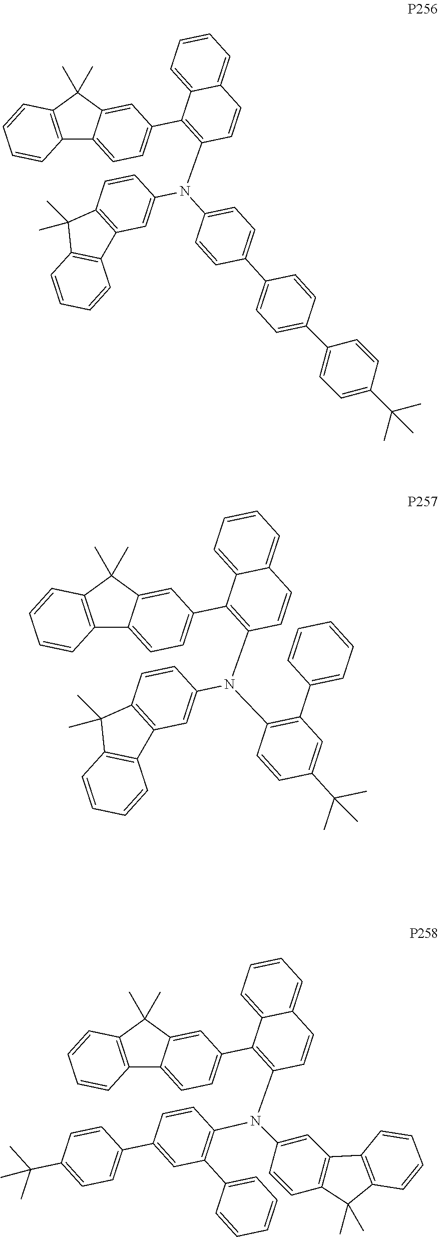

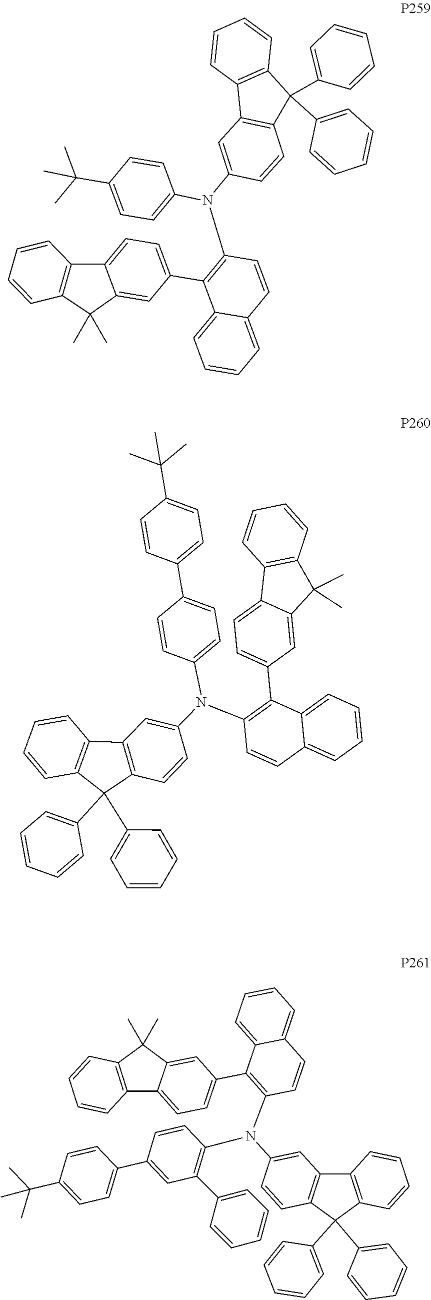 [SMILES: CC(C)(C)c1ccc(N(c2ccc3c(c2)-c2ccccc2C3(c2ccccc2)c2ccccc2)c2ccc3ccccc3c2-c2ccc3c(c2)C(C)(C)c2ccccc2-3)cc1 ; CC(C)(C)c1ccc(-c2ccc(N(c3ccc4c(c3)-c3ccccc3C4(c3ccccc3)c3ccccc3)c3ccc4ccccc4c3-c3ccc4c(c3)C(C)(C)c3ccccc3-4)cc2)cc1 ; CC(C)(C)c1ccc(-c2ccc(N(c3ccc4c(c3)-c3ccccc3C4(c3ccccc3)c3ccccc3)c3ccc4ccccc4c3-c3ccc4c(c3)C(C)(C)c3ccccc3-4)c(-c3ccccc3)c2)cc1]

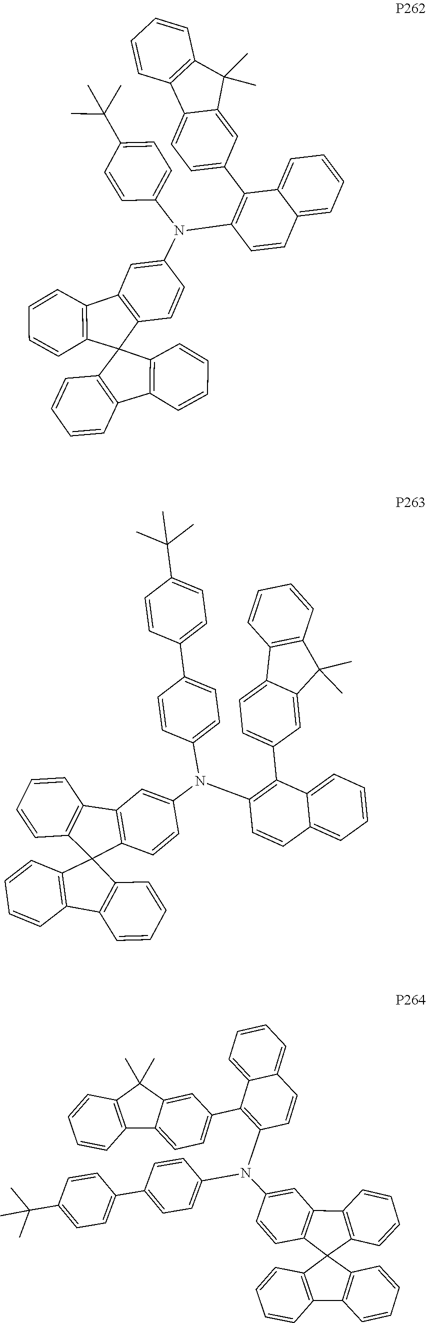 [SMILES: CC(C)(C)c1ccc(N(c2ccc3c(c2)-c2ccccc2C32c3ccccc3-c3ccccc32)c2ccc3ccccc3c2-c2ccc3c(c2)C(C)(C)c2ccccc2-3)cc1 ; CC(C)(C)c1ccc(-c2ccc(N(c3ccc4c(c3)-c3ccccc3C43c4ccccc4-c4ccccc43)c3ccc4ccccc4c3-c3ccc4c(c3)C(C)(C)c3ccccc3-4)cc2)cc1 ; CC(C)(C)c1ccc(-c2ccc(N(c3ccc4c(c3)-c3ccccc3C43c4ccccc4-c4ccccc43)c3ccc4ccccc4c3-c3ccc4c(c3)C(C)(C)c3ccccc3-4)cc2)cc1]

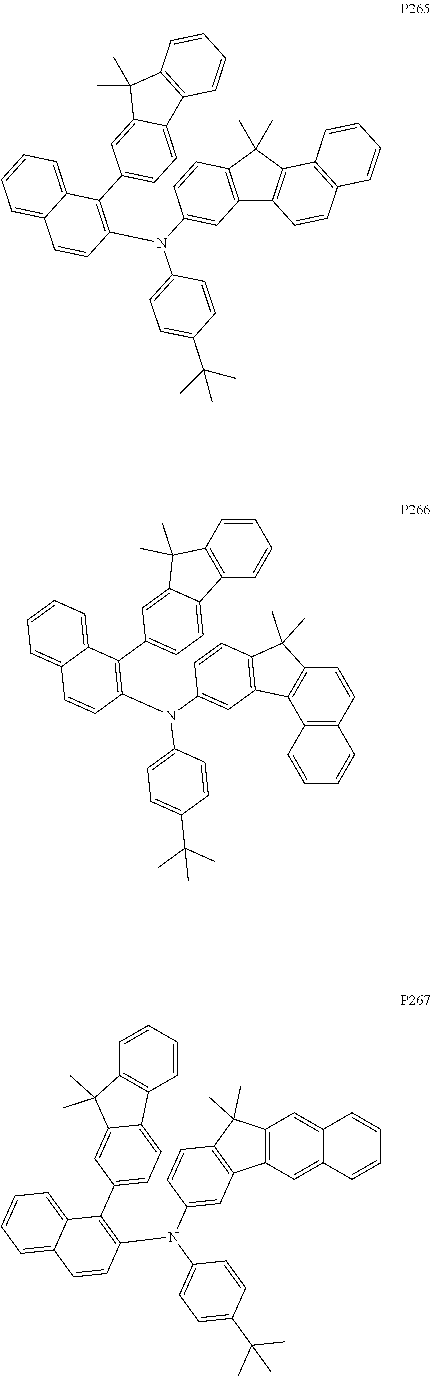 [SMILES: CC(C)(C)c1ccc(N(c2ccc3c(c2)-c2ccc4ccccc4c2C3(C)C)c2ccc3ccccc3c2-c2ccc3c(c2)C(C)(C)c2ccccc2-3)cc1 ; CC(C)(C)c1ccc(N(c2ccc3c(c2)-c2c(ccc4ccccc24)C3(C)C)c2ccc3ccccc3c2-c2ccc3c(c2)C(C)(C)c2ccccc2-3)cc1 ; CC(C)(C)c1ccc(N(c2ccc3c(c2)-c2cc4ccccc4cc2C3(C)C)c2ccc3ccccc3c2-c2ccc3c(c2)C(C)(C)c2ccccc2-3)cc1]

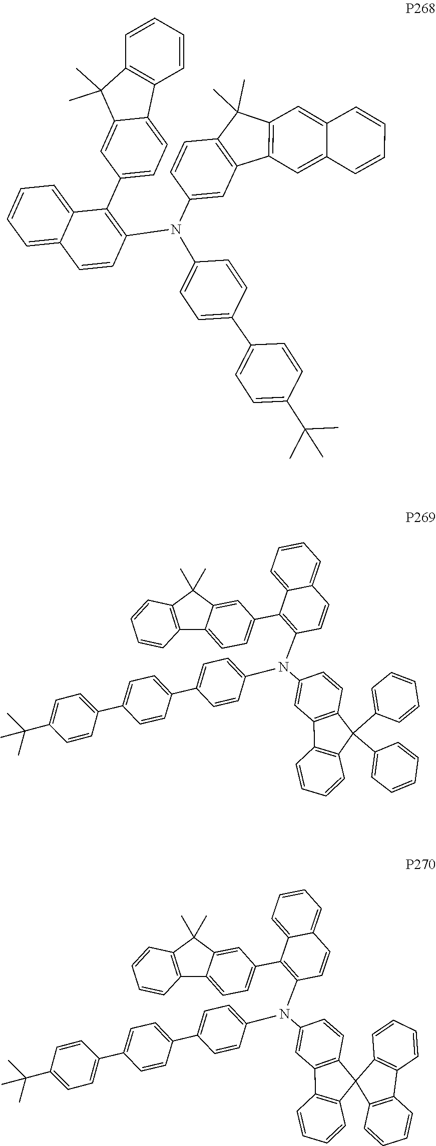 [SMILES: CC(C)(C)c1ccc(-c2ccc(N(c3ccc4c(c3)-c3cc5ccccc5cc3C4(C)C)c3ccc4ccccc4c3-c3ccc4c(c3)C(C)(C)c3ccccc3-4)cc2)cc1 ; CC(C)(C)c1ccc(-c2ccc(-c3ccc(N(c4ccc5c(c4)-c4ccccc4C5(c4ccccc4)c4ccccc4)c4ccc5ccccc5c4-c4ccc5c(c4)C(C)(C)c4ccccc4-5)cc3)cc2)cc1 ; CC(C)(C)c1ccc(-c2ccc(-c3ccc(N(c4ccc5c(c4)-c4ccccc4C54c5ccccc5-c5ccccc54)c4ccc5ccccc5c4-c4ccc5c(c4)C(C)(C)c4ccccc4-5)cc3)cc2)cc1]

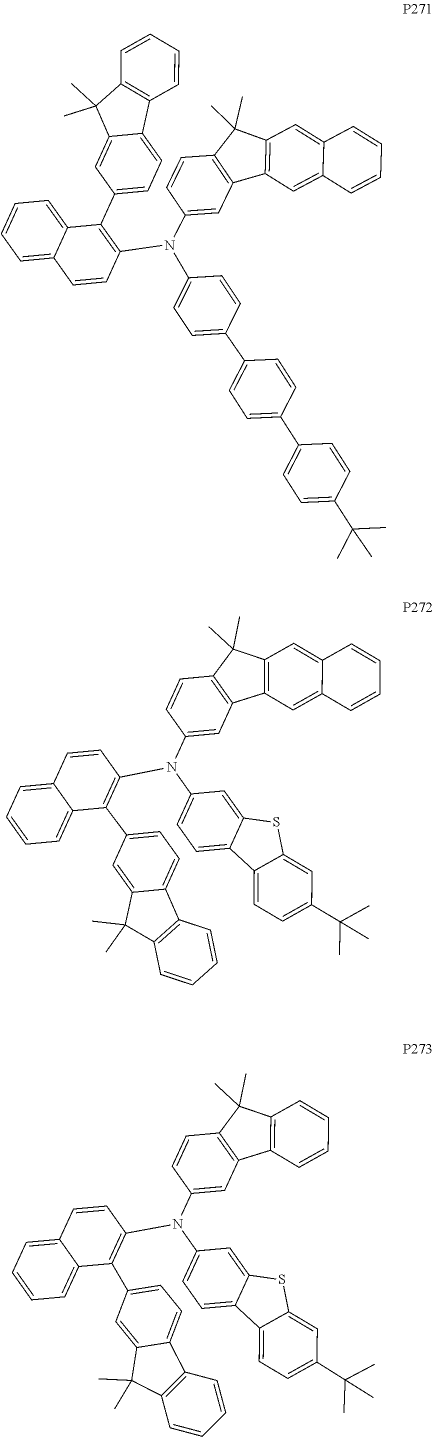 [SMILES: CC(C)(C)c1ccc(-c2ccc(-c3ccc(N(c4ccc5c(c4)-c4cc6ccccc6cc4C5(C)C)c4ccc5ccccc5c4-c4ccc5c(c4)C(C)(C)c4ccccc4-5)cc3)cc2)cc1 ; CC(C)(C)c1ccc2c(c1)sc1cc(N(c3ccc4c(c3)-c3cc5ccccc5cc3C4(C)C)c3ccc4ccccc4c3-c3ccc4c(c3)C(C)(C)c3ccccc3-4)ccc12 ; CC(C)(C)c1ccc2c(c1)sc1cc(N(c3ccc4c(c3)-c3ccccc3C4(C)C)c3ccc4ccccc4c3-c3ccc4c(c3)C(C)(C)c3ccccc3-4)ccc12]

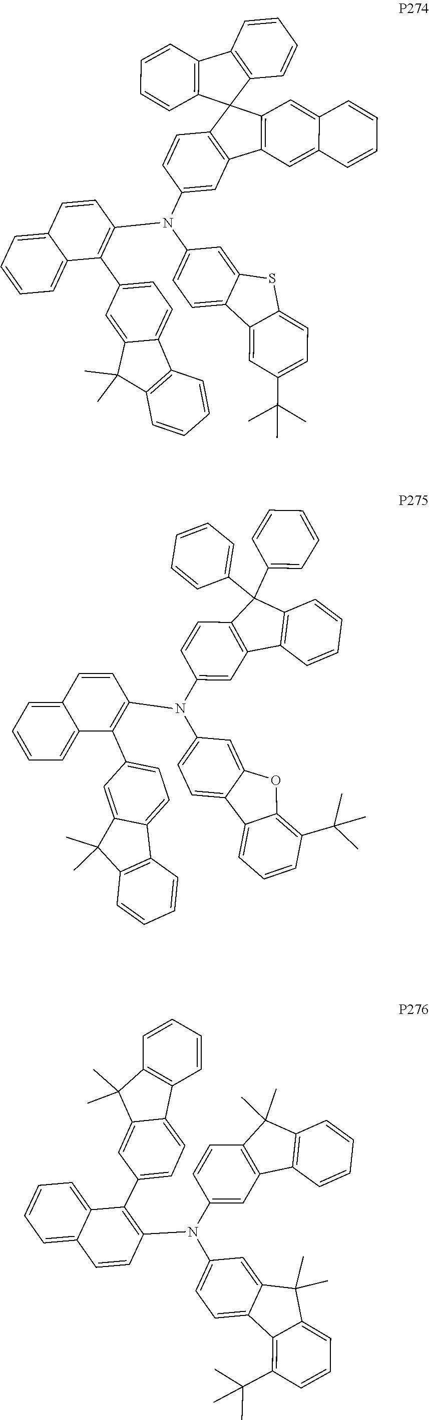 [SMILES: CC(C)(C)c1ccc2sc3cc(N(c4ccc5c(c4)-c4cc6ccccc6cc4C54c5ccccc5-c5ccccc54)c4ccc5ccccc5c4-c4ccc5c(c4)C(C)(C)c4ccccc4-5)ccc3c2c1 ; CC(C)(C)c1cccc2c1oc1cc(N(c3ccc4c(c3)-c3ccccc3C4(c3ccccc3)c3ccccc3)c3ccc4ccccc4c3-c3ccc4c(c3)C(C)(C)c3ccccc3-4)ccc12 ; CC(C)(C)c1cccc2c1-c1ccc(N(c3ccc4c(c3)-c3ccccc3C4(C)C)c3ccc4ccccc4c3-c3ccc4c(c3)C(C)(C)c3ccccc3-4)cc1C2(C)C]

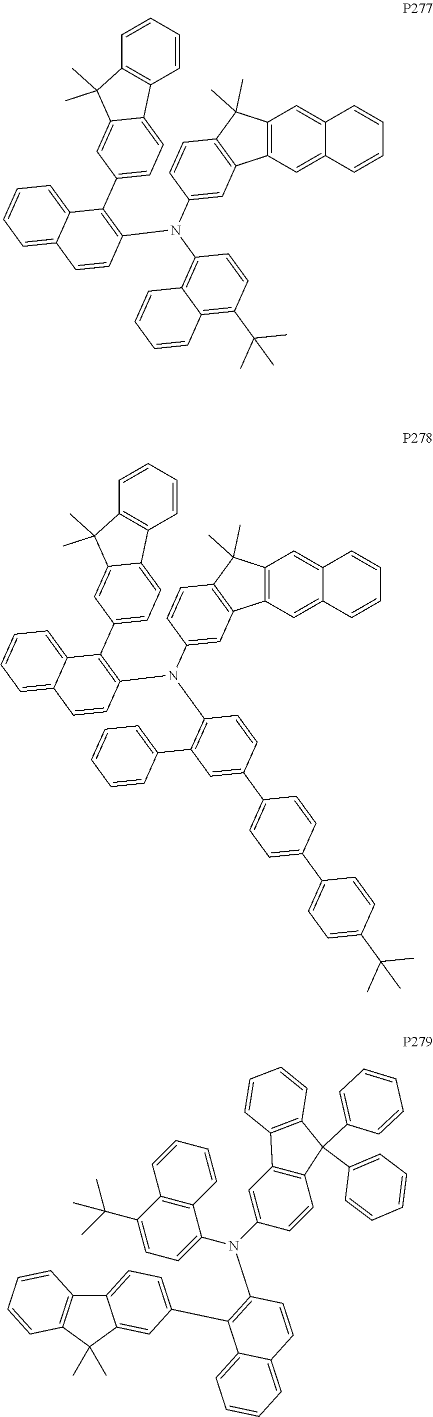 [SMILES: CC(C)(C)c1ccc(N(c2ccc3c(c2)-c2cc4ccccc4cc2C3(C)C)c2ccc3ccccc3c2-c2ccc3c(c2)C(C)(C)c2ccccc2-3)c2ccccc12 ; CC(C)(C)c1ccc(-c2ccc(-c3ccc(N(c4ccc5c(c4)-c4cc6ccccc6cc4C5(C)C)c4ccc5ccccc5c4-c4ccc5c(c4)C(C)(C)c4ccccc4-5)c(-c4ccccc4)c3)cc2)cc1 ; CC(C)(C)c1ccc(N(c2ccc3c(c2)-c2ccccc2C3(c2ccccc2)c2ccccc2)c2ccc3ccccc3c2-c2ccc3c(c2)C(C)(C)c2ccccc2-3)c2ccccc12]

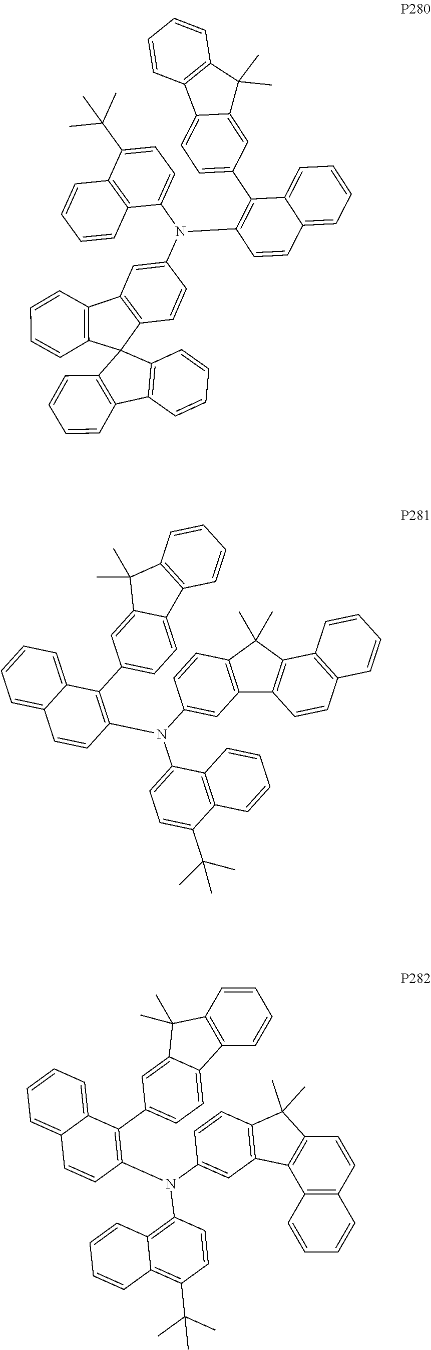 [SMILES: CC(C)(C)c1ccc(N(c2ccc3c(c2)-c2ccccc2C32c3ccccc3-c3ccccc32)c2ccc3ccccc3c2-c2ccc3c(c2)C(C)(C)c2ccccc2-3)c2ccccc12 ; CC(C)(C)c1ccc(N(c2ccc3c(c2)-c2ccc4ccccc4c2C3(C)C)c2ccc3ccccc3c2-c2ccc3c(c2)C(C)(C)c2ccccc2-3)c2ccccc12 ; CC(C)(C)c1ccc(N(c2ccc3c(c2)-c2c(ccc4ccccc24)C3(C)C)c2ccc3ccccc3c2-c2ccc3c(c2)C(C)(C)c2ccccc2-3)c2ccccc12]

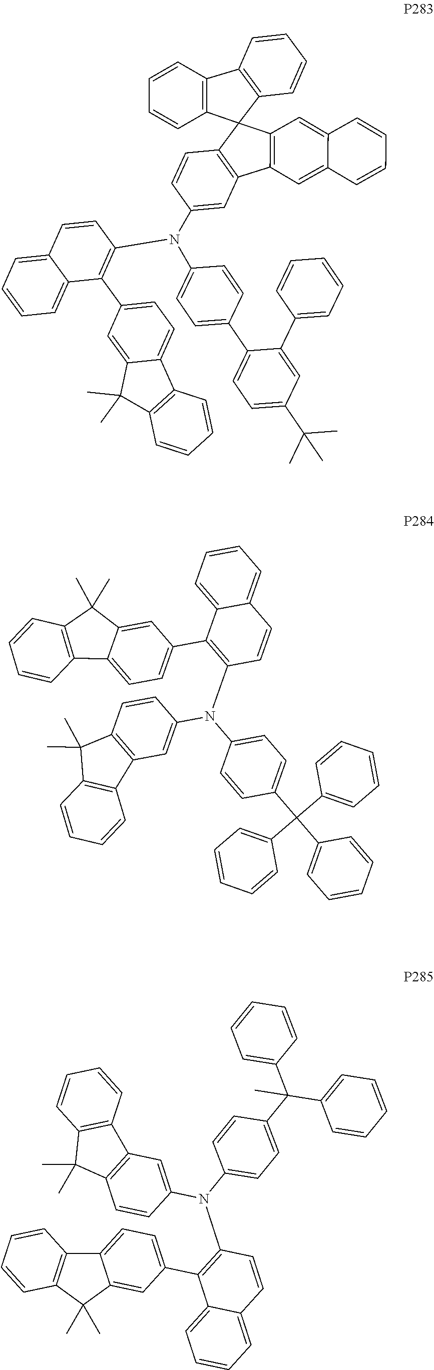 [SMILES: CC(C)(C)c1ccc(-c2ccc(N(c3ccc4c(c3)-c3cc5ccccc5cc3C43c4ccccc4-c4ccccc43)c3ccc4ccccc4c3-c3ccc4c(c3)C(C)(C)c3ccccc3-4)cc2)c(-c2ccccc2)c1 ; CC1(C)c2ccccc2-c2cc(N(c3ccc(C(c4ccccc4)(c4ccccc4)c4ccccc4)cc3)c3ccc4ccccc4c3-c3ccc4c(c3)C(C)(C)c3ccccc3-4)ccc21 ; CC1(C)c2ccccc2-c2cc(N(c3ccc(C(C)(c4ccccc4)c4ccccc4)cc3)c3ccc4ccccc4c3-c3ccc4c(c3)C(C)(C)c3ccccc3-4)ccc21]

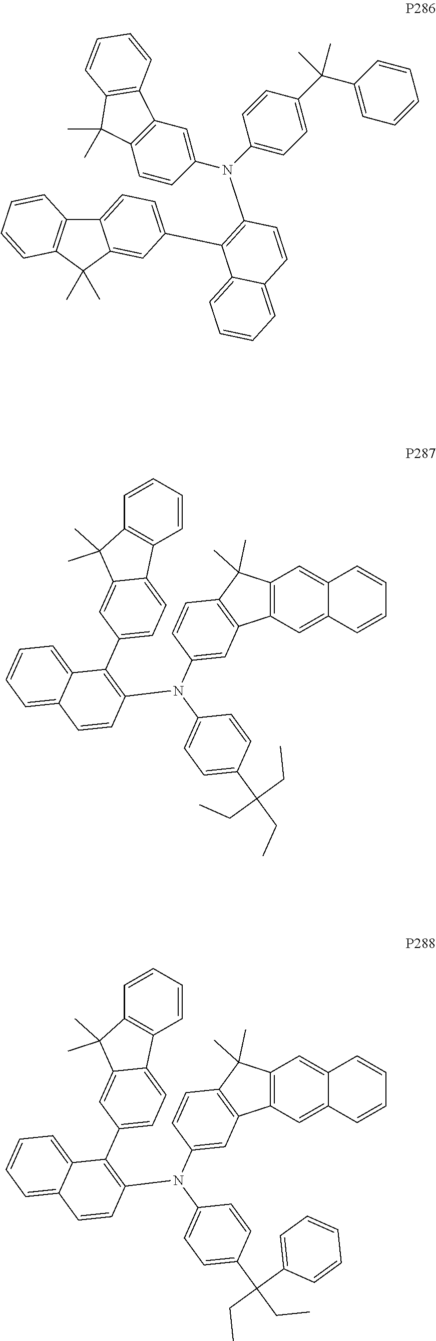 [SMILES: CC(C)(c1ccccc1)c1ccc(N(c2ccc3c(c2)-c2ccccc2C3(C)C)c2ccc3ccccc3c2-c2ccc3c(c2)C(C)(C)c2ccccc2-3)cc1 ; CCC(CC)(CC)c1ccc(N(c2ccc3c(c2)-c2cc4ccccc4cc2C3(C)C)c2ccc3ccccc3c2-c2ccc3c(c2)C(C)(C)c2ccccc2-3)cc1 ; CCC(CC)(c1ccccc1)c1ccc(N(c2ccc3c(c2)-c2cc4ccccc4cc2C3(C)C)c2ccc3ccccc3c2-c2ccc3c(c2)C(C)(C)c2ccccc2-3)cc1]

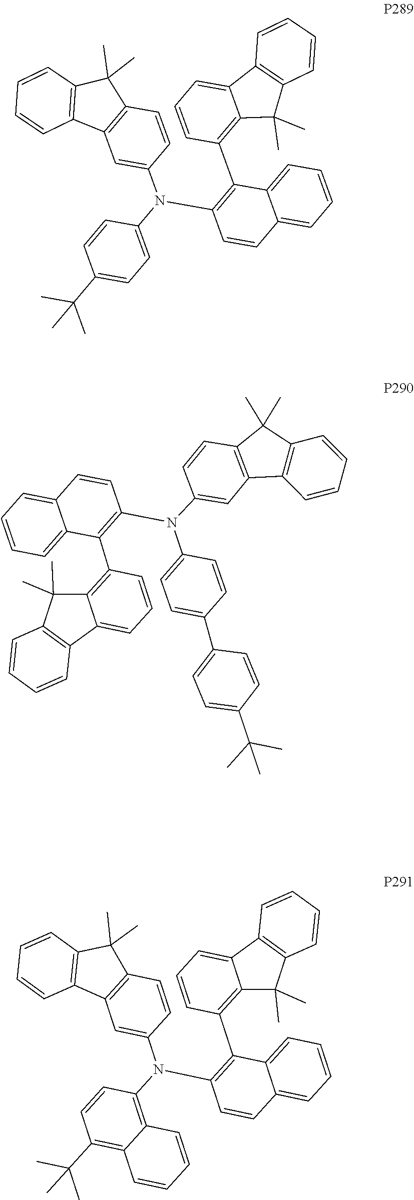 [SMILES: CC(C)(C)c1ccc(N(c2ccc3c(c2)-c2ccccc2C3(C)C)c2ccc3ccccc3c2-c2cccc3c2C(C)(C)c2ccccc2-3)cc1 ; CC(C)(C)c1ccc(-c2ccc(N(c3ccc4c(c3)-c3ccccc3C4(C)C)c3ccc4ccccc4c3-c3cccc4c3C(C)(C)c3ccccc3-4)cc2)cc1 ; CC(C)(C)c1ccc(N(c2ccc3c(c2)-c2ccccc2C3(C)C)c2ccc3ccccc3c2-c2cccc3c2C(C)(C)c2ccccc2-3)c2ccccc12]

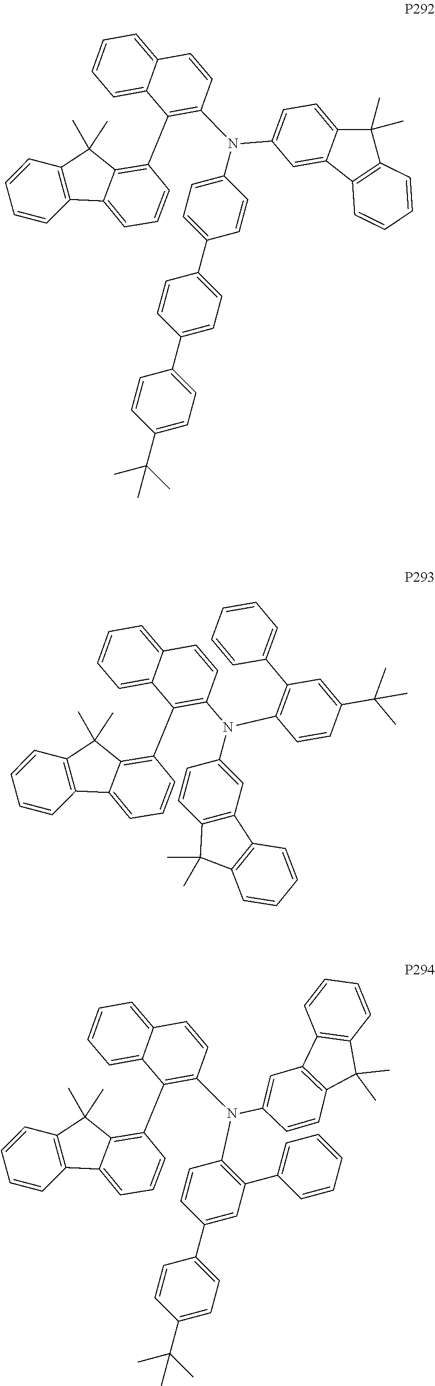 [SMILES: CC(C)(C)c1ccc(-c2ccc(-c3ccc(N(c4ccc5c(c4)-c4ccccc4C5(C)C)c4ccc5ccccc5c4-c4cccc5c4C(C)(C)c4ccccc4-5)cc3)cc2)cc1 ; CC(C)(C)c1ccc(N(c2ccc3c(c2)-c2ccccc2C3(C)C)c2ccc3ccccc3c2-c2cccc3c2C(C)(C)c2ccccc2-3)c(-c2ccccc2)c1 ; CC(C)(C)c1ccc(-c2ccc(N(c3ccc4c(c3)-c3ccccc3C4(C)C)c3ccc4ccccc4c3-c3cccc4c3C(C)(C)c3ccccc3-4)c(-c3ccccc3)c2)cc1]

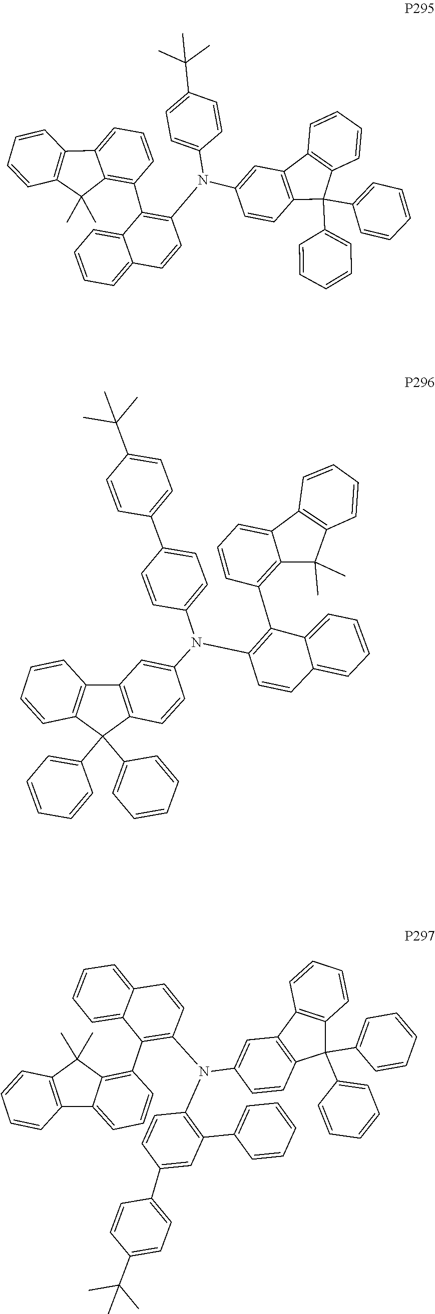 [SMILES: CC(C)(C)c1ccc(N(c2ccc3c(c2)-c2ccccc2C3(c2ccccc2)c2ccccc2)c2ccc3ccccc3c2-c2cccc3c2C(C)(C)c2ccccc2-3)cc1 ; CC(C)(C)c1ccc(-c2ccc(N(c3ccc4c(c3)-c3ccccc3C4(c3ccccc3)c3ccccc3)c3ccc4ccccc4c3-c3cccc4c3C(C)(C)c3ccccc3-4)cc2)cc1 ; CC(C)(C)c1ccc(-c2ccc(N(c3ccc4c(c3)-c3ccccc3C4(c3ccccc3)c3ccccc3)c3ccc4ccccc4c3-c3cccc4c3C(C)(C)c3ccccc3-4)c(-c3ccccc3)c2)cc1]

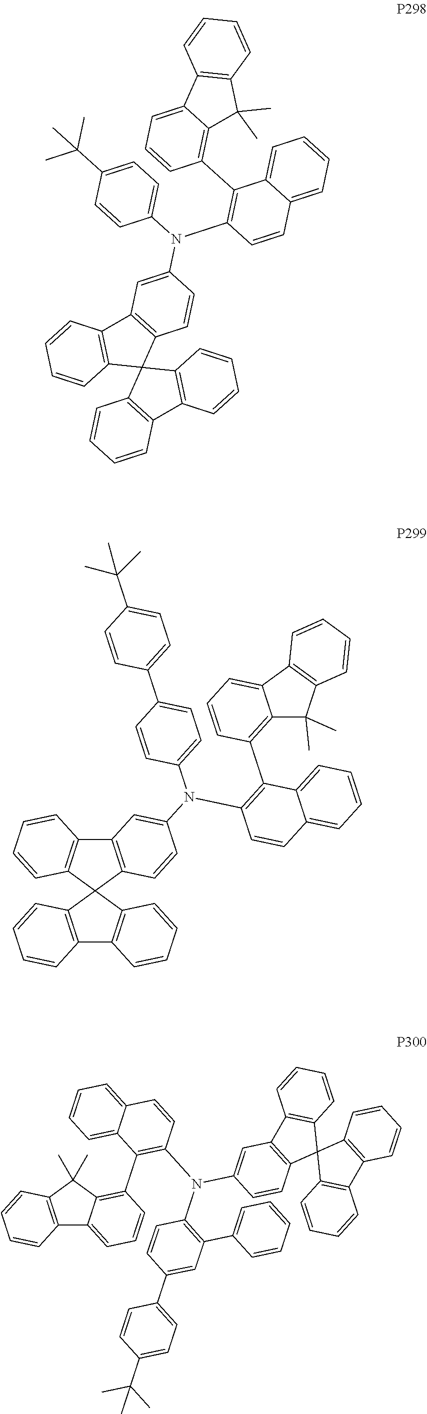 [SMILES: CC(C)(C)c1ccc(N(c2ccc3c(c2)-c2ccccc2C32c3ccccc3-c3ccccc32)c2ccc3ccccc3c2-c2cccc3c2C(C)(C)c2ccccc2-3)cc1 ; CC(C)(C)c1ccc(-c2ccc(N(c3ccc4c(c3)-c3ccccc3C43c4ccccc4-c4ccccc43)c3ccc4ccccc4c3-c3cccc4c3C(C)(C)c3ccccc3-4)cc2)cc1 ; CC(C)(C)c1ccc(-c2ccc(N(c3ccc4c(c3)-c3ccccc3C43c4ccccc4-c4ccccc43)c3ccc4ccccc4c3-c3cccc4c3C(C)(C)c3ccccc3-4)c(-c3ccccc3)c2)cc1]

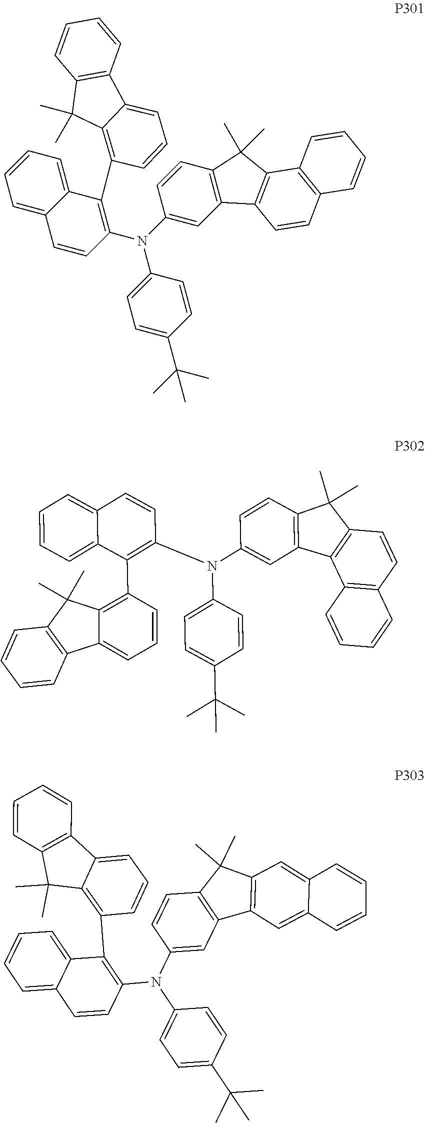 [SMILES: CC(C)(C)c1ccc(N(c2ccc3c(c2)-c2ccc4ccccc4c2C3(C)C)c2ccc3ccccc3c2-c2cccc3c2C(C)(C)c2ccccc2-3)cc1 ; CC(C)(C)c1ccc(N(c2ccc3c(c2)-c2c(ccc4ccccc24)C3(C)C)c2ccc3ccccc3c2-c2cccc3c2C(C)(C)c2ccccc2-3)cc1 ; CC(C)(C)c1ccc(N(c2ccc3c(c2)-c2cc4ccccc4cc2C3(C)C)c2ccc3ccccc3c2-c2cccc3c2C(C)(C)c2ccccc2-3)cc1]

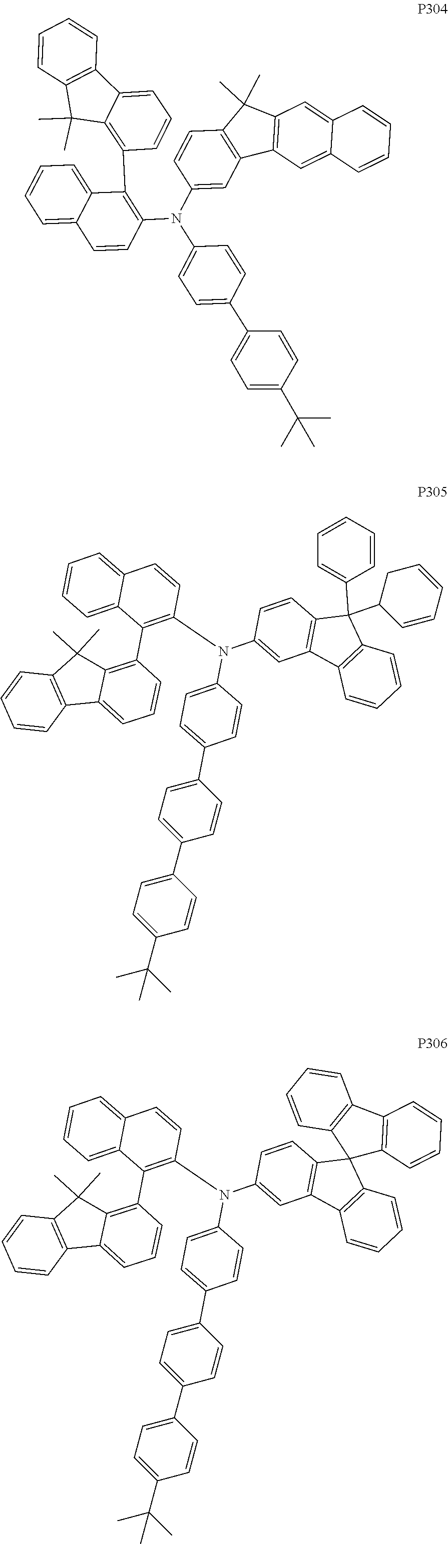 [SMILES: CC(C)(C)c1ccc(-c2ccc(N(c3ccc4c(c3)-c3cc5ccccc5cc3C4(C)C)c3ccc4ccccc4c3-c3cccc4c3C(C)(C)c3ccccc3-4)cc2)cc1 ; CC(C)(C)c1ccc(-c2ccc(-c3ccc(N(c4ccc5c(c4)-c4ccccc4C5(c4ccccc4)C4C=CC=CC4)c4ccc5ccccc5c4-c4cccc5c4C(C)(C)c4ccccc4-5)cc3)cc2)cc1 ; CC(C)(C)c1ccc(-c2ccc(-c3ccc(N(c4ccc5c(c4)-c4ccccc4C54c5ccccc5-c5ccccc54)c4ccc5ccccc5c4-c4cccc5c4C(C)(C)c4ccccc4-5)cc3)cc2)cc1]

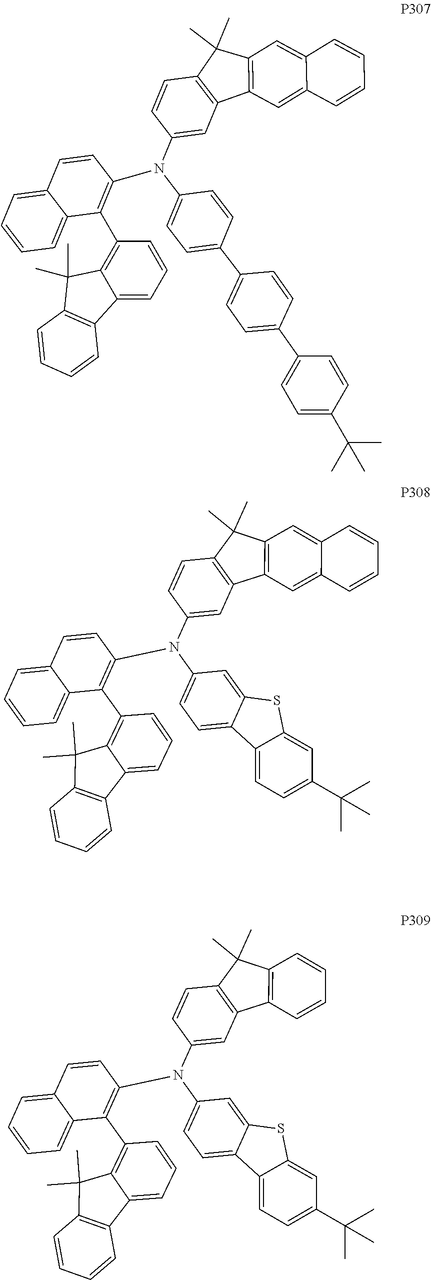 [SMILES: CC(C)(C)c1ccc(-c2ccc(-c3ccc(N(c4ccc5c(c4)-c4cc6ccccc6cc4C5(C)C)c4ccc5ccccc5c4-c4cccc5c4C(C)(C)c4ccccc4-5)cc3)cc2)cc1 ; CC(C)(C)c1ccc2c(c1)sc1cc(N(c3ccc4c(c3)-c3cc5ccccc5cc3C4(C)C)c3ccc4ccccc4c3-c3cccc4c3C(C)(C)c3ccccc3-4)ccc12 ; CC(C)(C)c1ccc2c(c1)sc1cc(N(c3ccc4c(c3)-c3ccccc3C4(C)C)c3ccc4ccccc4c3-c3cccc4c3C(C)(C)c3ccccc3-4)ccc12]

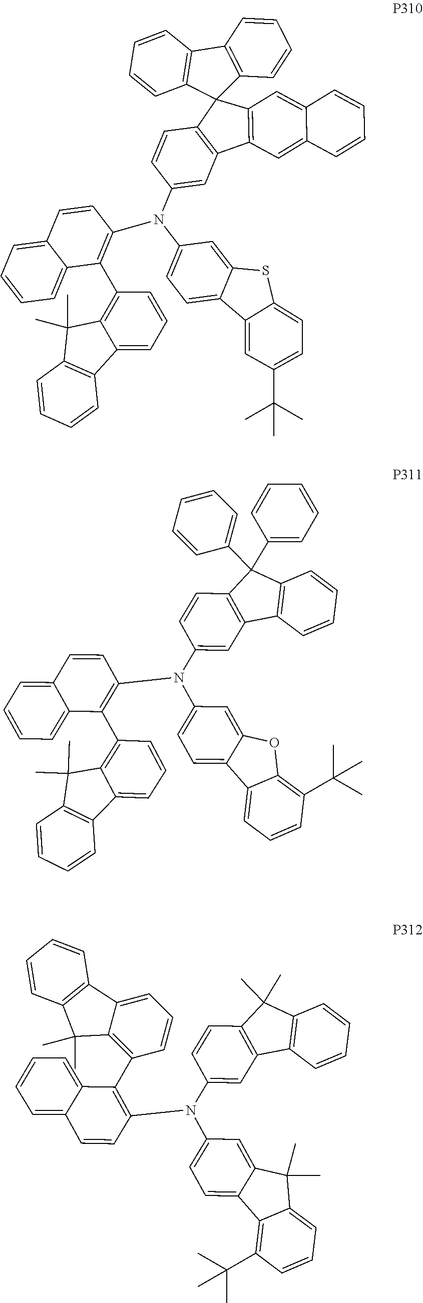 [SMILES: CC(C)(C)c1ccc2sc3cc(N(c4ccc5c(c4)-c4cc6ccccc6cc4C54c5ccccc5-c5ccccc54)c4ccc5ccccc5c4-c4cccc5c4C(C)(C)c4ccccc4-5)ccc3c2c1 ; CC(C)(C)c1cccc2c1oc1cc(N(c3ccc4c(c3)-c3ccccc3C4(c3ccccc3)c3ccccc3)c3ccc4ccccc4c3-c3cccc4c3C(C)(C)c3ccccc3-4)ccc12 ; CC(C)(C)c1cccc2c1-c1ccc(N(c3ccc4c(c3)-c3ccccc3C4(C)C)c3ccc4ccccc4c3-c3cccc4c3C(C)(C)c3ccccc3-4)cc1C2(C)C]

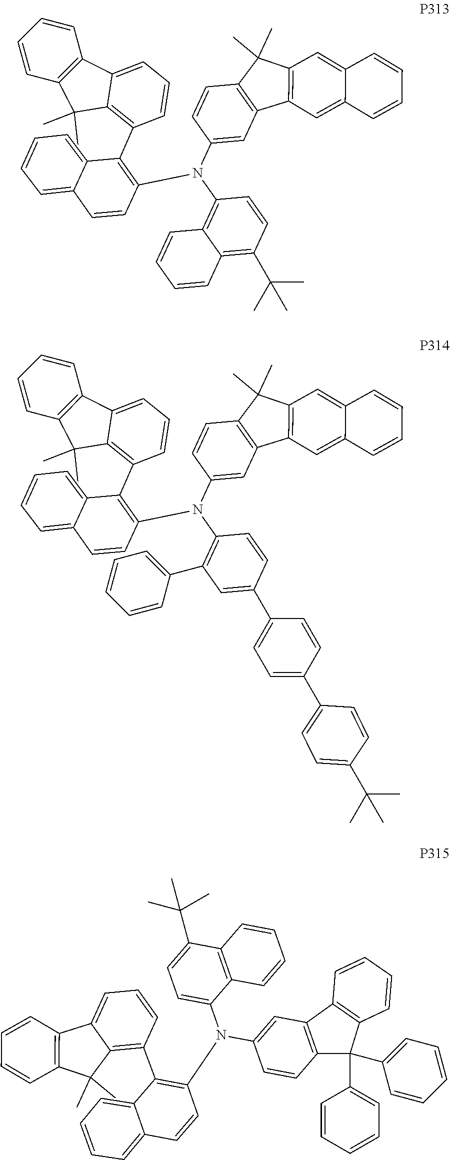 [SMILES: CC(C)(C)c1ccc(N(c2ccc3c(c2)-c2cc4ccccc4cc2C3(C)C)c2ccc3ccccc3c2-c2cccc3c2C(C)(C)c2ccccc2-3)c2ccccc12 ; CC(C)(C)c1ccc(-c2ccc(-c3ccc(N(c4ccc5c(c4)-c4cc6ccccc6cc4C5(C)C)c4ccc5ccccc5c4-c4cccc5c4C(C)(C)c4ccccc4-5)c(-c4ccccc4)c3)cc2)cc1 ; CC(C)(C)c1ccc(N(c2ccc3c(c2)-c2ccccc2C3(c2ccccc2)c2ccccc2)c2ccc3ccccc3c2-c2cccc3c2C(C)(C)c2ccccc2-3)c2ccccc12]

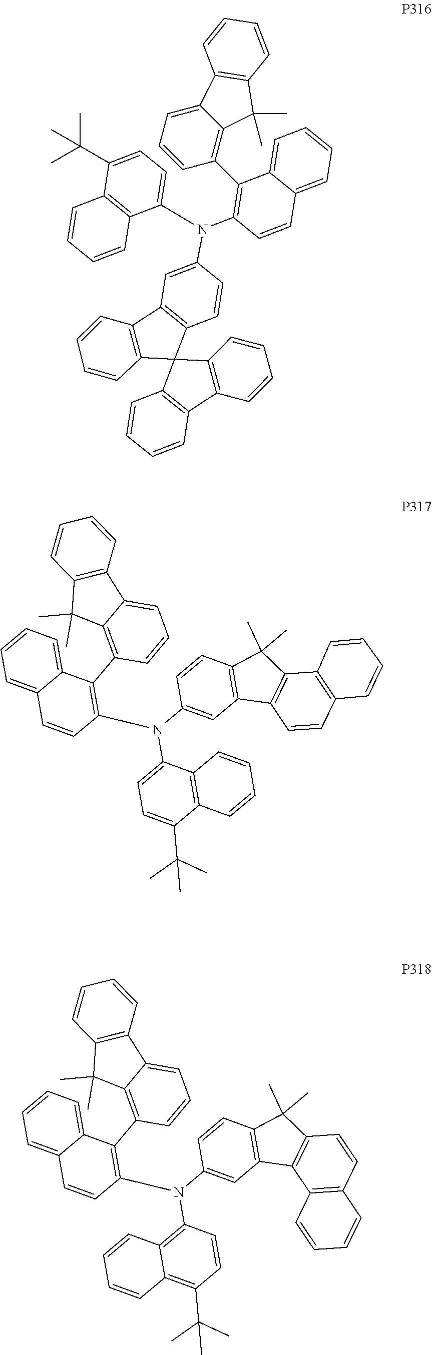 [SMILES: CC(C)(C)c1ccc(N(c2ccc3c(c2)-c2ccccc2C32c3ccccc3-c3ccccc32)c2ccc3ccccc3c2-c2cccc3c2C(C)(C)c2ccccc2-3)c2ccccc12 ; CC(C)(C)c1ccc(N(c2ccc3c(c2)-c2ccc4ccccc4c2C3(C)C)c2ccc3ccccc3c2-c2cccc3c2C(C)(C)c2ccccc2-3)c2ccccc12 ; CC(C)(C)c1ccc(N(c2ccc3c(c2)-c2c(ccc4ccccc24)C3(C)C)c2ccc3ccccc3c2-c2cccc3c2C(C)(C)c2ccccc2-3)c2ccccc12]

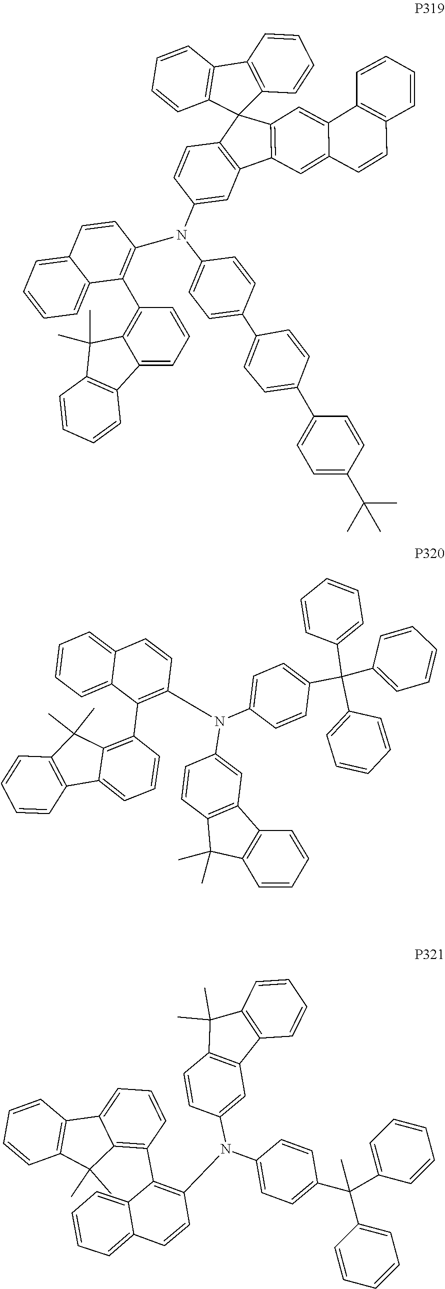 [SMILES: CC(C)(C)c1ccc(-c2ccc(-c3ccc(N(c4ccc5c(c4)-c4cc6ccc7ccccc7c6cc4C54c5ccccc5-c5ccccc54)c4ccc5ccccc5c4-c4cccc5c4C(C)(C)c4ccccc4-5)cc3)cc2)cc1 ; CC1(C)c2ccccc2-c2cc(N(c3ccc(C(c4ccccc4)(c4ccccc4)c4ccccc4)cc3)c3ccc4ccccc4c3-c3cccc4c3C(C)(C)c3ccccc3-4)ccc21 ; CC1(C)c2ccccc2-c2cc(N(c3ccc(C(C)(c4ccccc4)c4ccccc4)cc3)c3ccc4ccccc4c3-c3cccc4c3C(C)(C)c3ccccc3-4)ccc21]

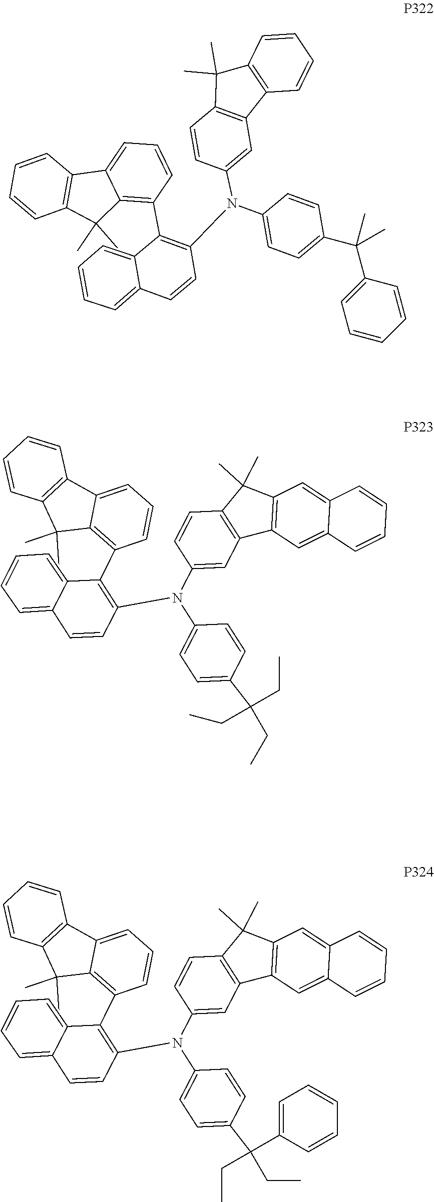 [SMILES: CC(C)(c1ccccc1)c1ccc(N(c2ccc3c(c2)-c2ccccc2C3(C)C)c2ccc3ccccc3c2-c2cccc3c2C(C)(C)c2ccccc2-3)cc1 ; CCC(CC)(CC)c1ccc(N(c2ccc3c(c2)-c2cc4ccccc4cc2C3(C)C)c2ccc3ccccc3c2-c2cccc3c2C(C)(C)c2ccccc2-3)cc1 ; CCC(CC)(c1ccccc1)c1ccc(N(c2ccc3c(c2)-c2cc4ccccc4cc2C3(C)C)c2ccc3ccccc3c2-c2cccc3c2C(C)(C)c2ccccc2-3)cc1]

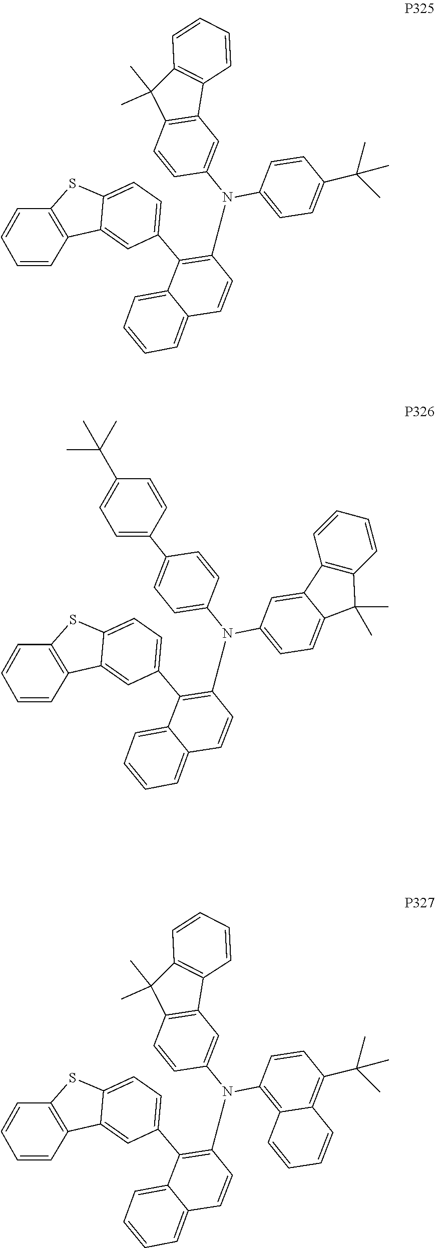 [SMILES: CC(C)(C)c1ccc(N(c2ccc3c(c2)-c2ccccc2C3(C)C)c2ccc3ccccc3c2-c2ccc3sc4ccccc4c3c2)cc1 ; CC(C)(C)c1ccc(-c2ccc(N(c3ccc4c(c3)-c3ccccc3C4(C)C)c3ccc4ccccc4c3-c3ccc4sc5ccccc5c4c3)cc2)cc1 ; CC(C)(C)c1ccc(N(c2ccc3c(c2)-c2ccccc2C3(C)C)c2ccc3ccccc3c2-c2ccc3sc4ccccc4c3c2)c2ccccc12]

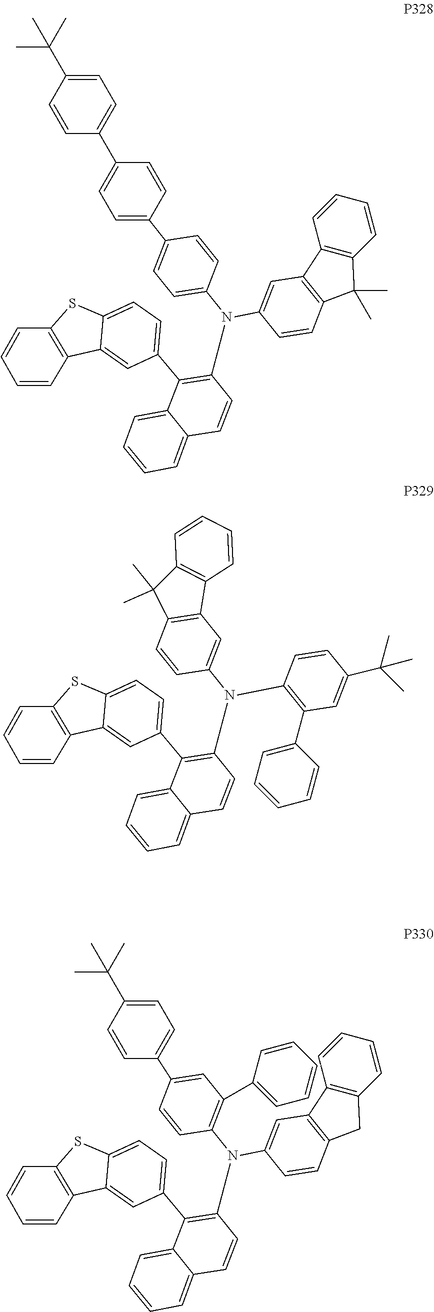 [SMILES: CC(C)(C)c1ccc(-c2ccc(-c3ccc(N(c4ccc5c(c4)-c4ccccc4C5(C)C)c4ccc5ccccc5c4-c4ccc5sc6ccccc6c5c4)cc3)cc2)cc1 ; CC(C)(C)c1ccc(N(c2ccc3c(c2)-c2ccccc2C3(C)C)c2ccc3ccccc3c2-c2ccc3sc4ccccc4c3c2)c(-c2ccccc2)c1 ; CC(C)(C)c1ccc(-c2ccc(N(c3ccc4c(c3)-c3ccccc3C4)c3ccc4ccccc4c3-c3ccc4sc5ccccc5c4c3)c(-c3ccccc3)c2)cc1]

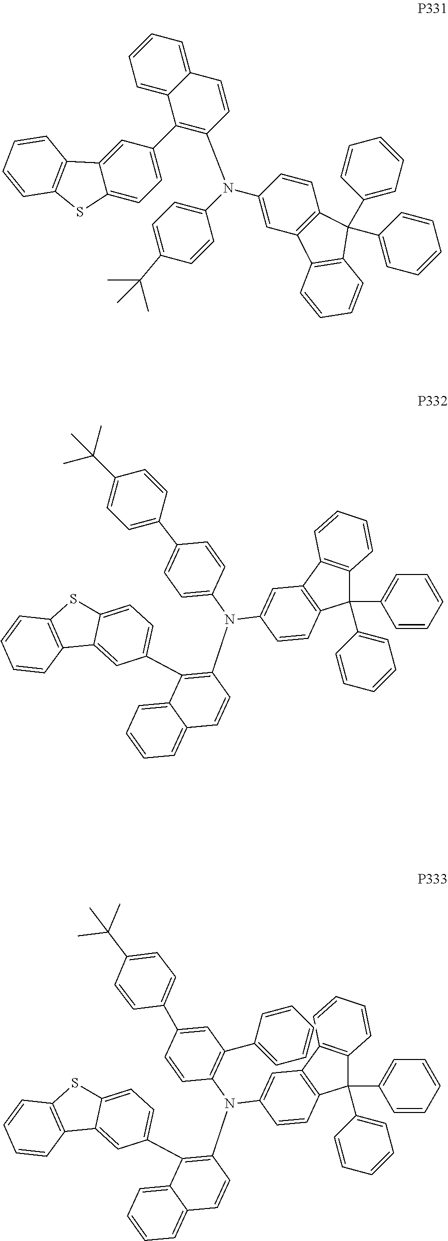 [SMILES: CC(C)(C)c1ccc(N(c2ccc3c(c2)-c2ccccc2C3(c2ccccc2)c2ccccc2)c2ccc3ccccc3c2-c2ccc3sc4ccccc4c3c2)cc1 ; CC(C)(C)c1ccc(-c2ccc(N(c3ccc4c(c3)-c3ccccc3C4(c3ccccc3)c3ccccc3)c3ccc4ccccc4c3-c3ccc4sc5ccccc5c4c3)cc2)cc1 ; CC(C)(C)c1ccc(-c2ccc(N(c3ccc4c(c3)-c3ccccc3C4(c3ccccc3)c3ccccc3)c3ccc4ccccc4c3-c3ccc4sc5ccccc5c4c3)c(-c3ccccc3)c2)cc1]

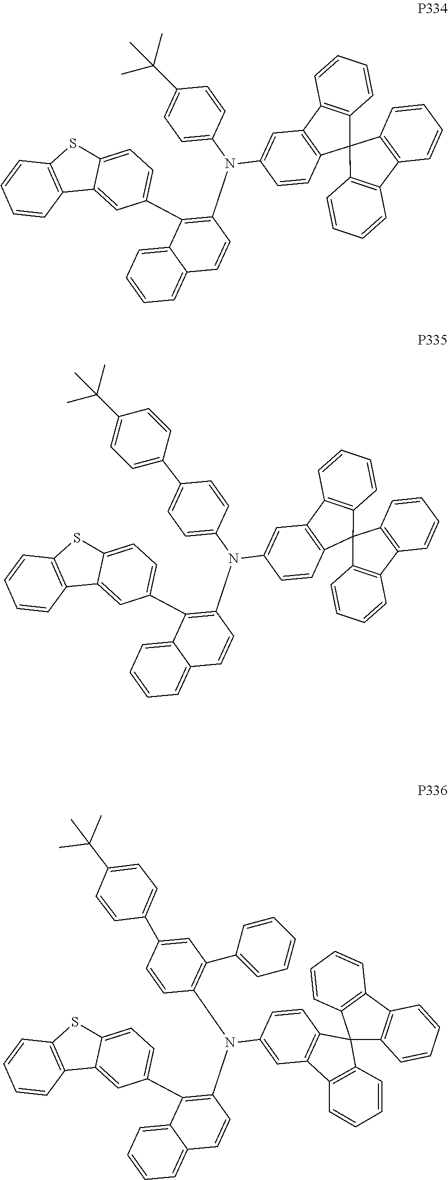 [SMILES: CC(C)(C)c1ccc(N(c2ccc3c(c2)-c2ccccc2C32c3ccccc3-c3ccccc32)c2ccc3ccccc3c2-c2ccc3sc4ccccc4c3c2)cc1 ; CC(C)(C)c1ccc(-c2ccc(N(c3ccc4c(c3)-c3ccccc3C43c4ccccc4-c4ccccc43)c3ccc4ccccc4c3-c3ccc4sc5ccccc5c4c3)cc2)cc1 ; CC(C)(C)c1ccc(-c2ccc(N(c3ccc4c(c3)-c3ccccc3C43c4ccccc4-c4ccccc43)c3ccc4ccccc4c3-c3ccc4sc5ccccc5c4c3)c(-c3ccccc3)c2)cc1]

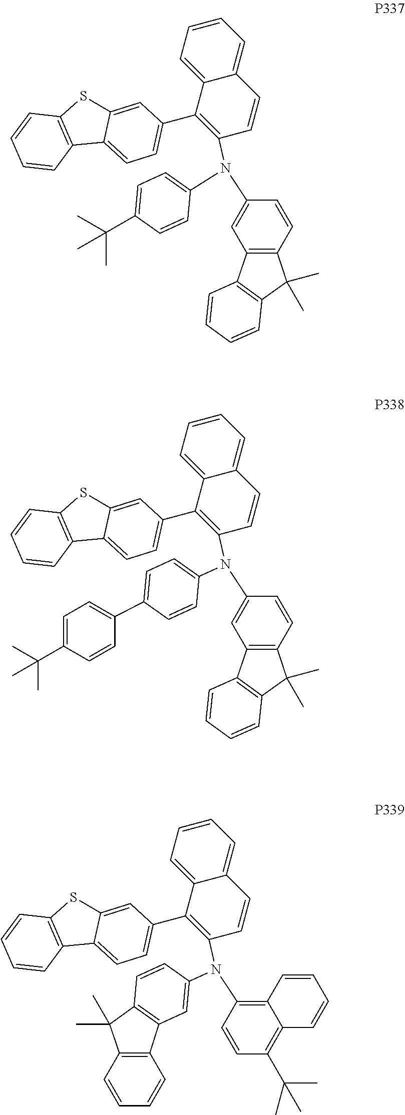 [SMILES: CC(C)(C)c1ccc(N(c2ccc3c(c2)-c2ccccc2C3(C)C)c2ccc3ccccc3c2-c2ccc3c(c2)sc2ccccc23)cc1 ; CC(C)(C)c1ccc(-c2ccc(N(c3ccc4c(c3)-c3ccccc3C4(C)C)c3ccc4ccccc4c3-c3ccc4c(c3)sc3ccccc34)cc2)cc1 ; CC(C)(C)c1ccc(N(c2ccc3c(c2)-c2ccccc2C3(C)C)c2ccc3ccccc3c2-c2ccc3c(c2)sc2ccccc23)c2ccccc12]

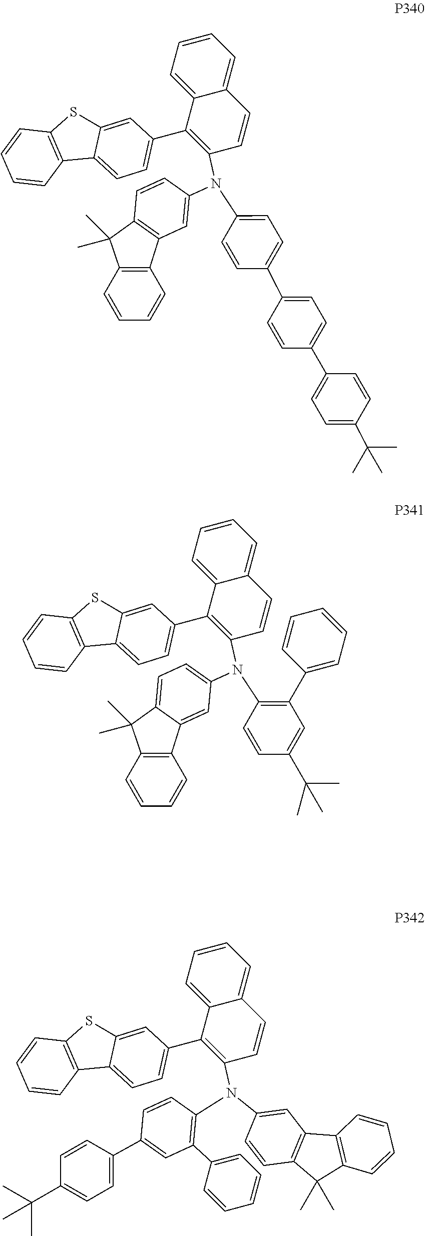

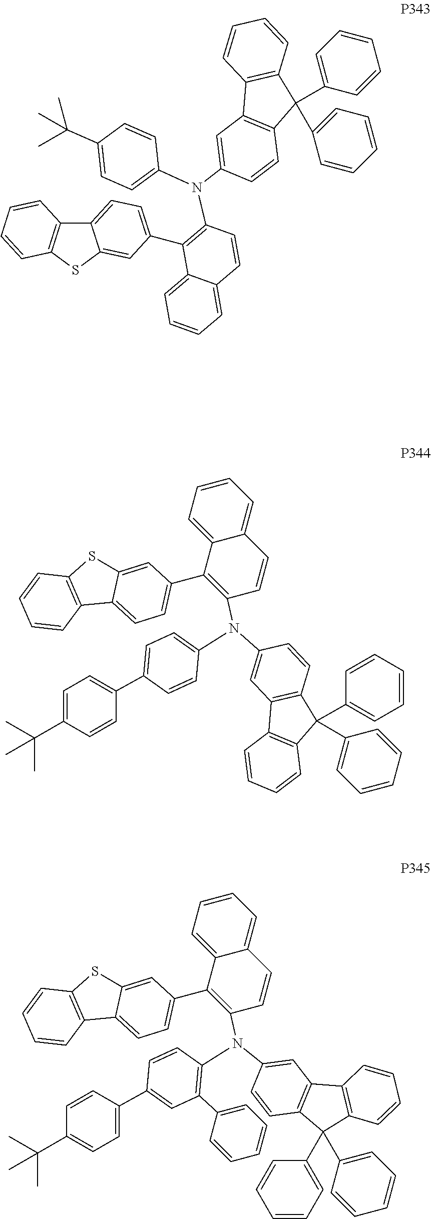 [SMILES: CC(C)(C)c1ccc(N(c2ccc3c(c2)-c2ccccc2C3(c2ccccc2)c2ccccc2)c2ccc3ccccc3c2-c2ccc3c(c2)sc2ccccc23)cc1 ; CC(C)(C)c1ccc(-c2ccc(N(c3ccc4c(c3)-c3ccccc3C4(c3ccccc3)c3ccccc3)c3ccc4ccccc4c3-c3ccc4c(c3)sc3ccccc34)cc2)cc1 ; CC(C)(C)c1ccc(-c2ccc(N(c3ccc4c(c3)-c3ccccc3C4(c3ccccc3)c3ccccc3)c3ccc4ccccc4c3-c3ccc4c(c3)sc3ccccc34)c(-c3ccccc3)c2)cc1]

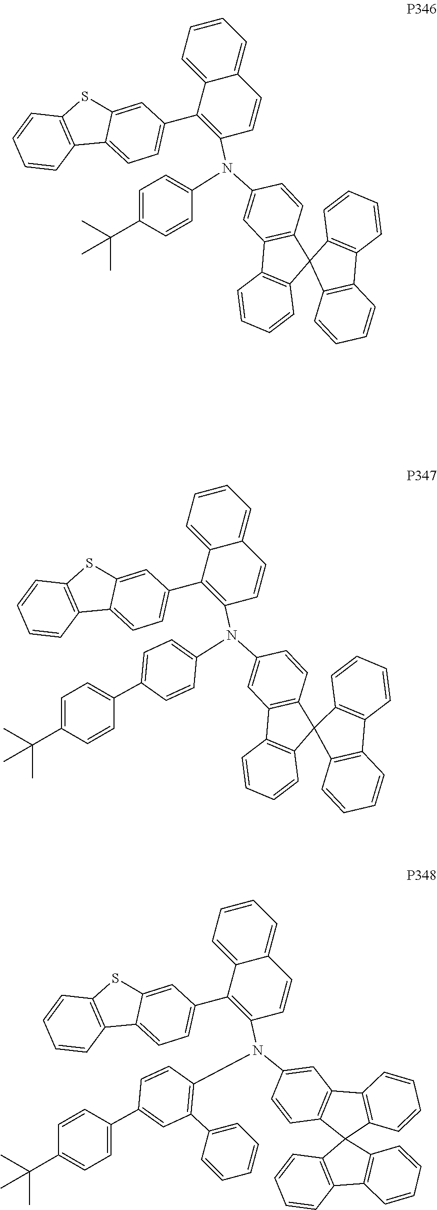 [SMILES: CC(C)(C)c1ccc(N(c2ccc3c(c2)-c2ccccc2C32c3ccccc3-c3ccccc32)c2ccc3ccccc3c2-c2ccc3c(c2)sc2ccccc23)cc1 ; CC(C)(C)c1ccc(-c2ccc(N(c3ccc4c(c3)-c3ccccc3C43c4ccccc4-c4ccccc43)c3ccc4ccccc4c3-c3ccc4c(c3)sc3ccccc34)cc2)cc1 ; CC(C)(C)c1ccc(-c2ccc(N(c3ccc4c(c3)-c3ccccc3C43c4ccccc4-c4ccccc43)c3ccc4ccccc4c3-c3ccc4c(c3)sc3ccccc34)c(-c3ccccc3)c2)cc1]

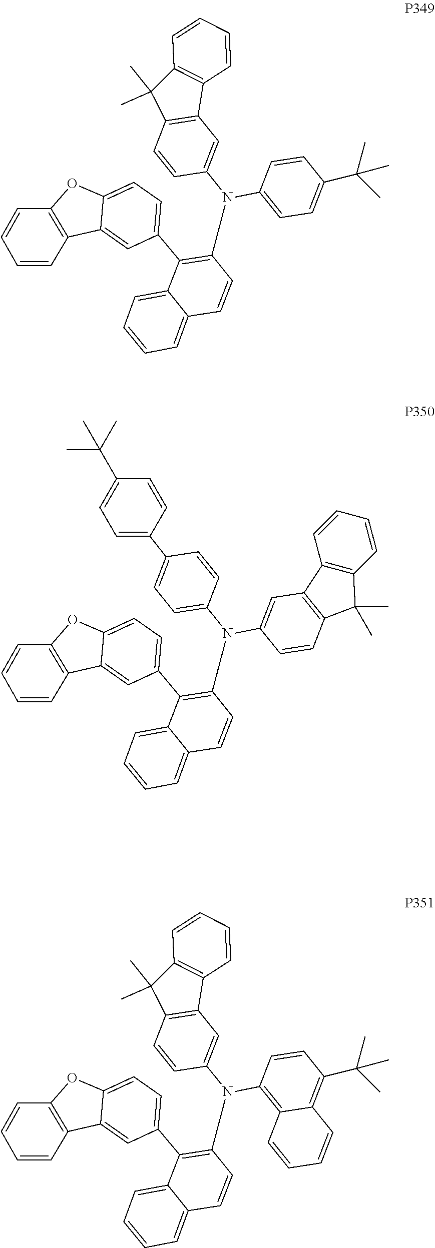 [SMILES: CC(C)(C)c1ccc(N(c2ccc3c(c2)-c2ccccc2C3(C)C)c2ccc3ccccc3c2-c2ccc3oc4ccccc4c3c2)cc1 ; CC(C)(C)c1ccc(-c2ccc(N(c3ccc4c(c3)-c3ccccc3C4(C)C)c3ccc4ccccc4c3-c3ccc4oc5ccccc5c4c3)cc2)cc1 ; CC(C)(C)c1ccc(N(c2ccc3c(c2)-c2ccccc2C3(C)C)c2ccc3ccccc3c2-c2ccc3oc4ccccc4c3c2)c2ccccc12]

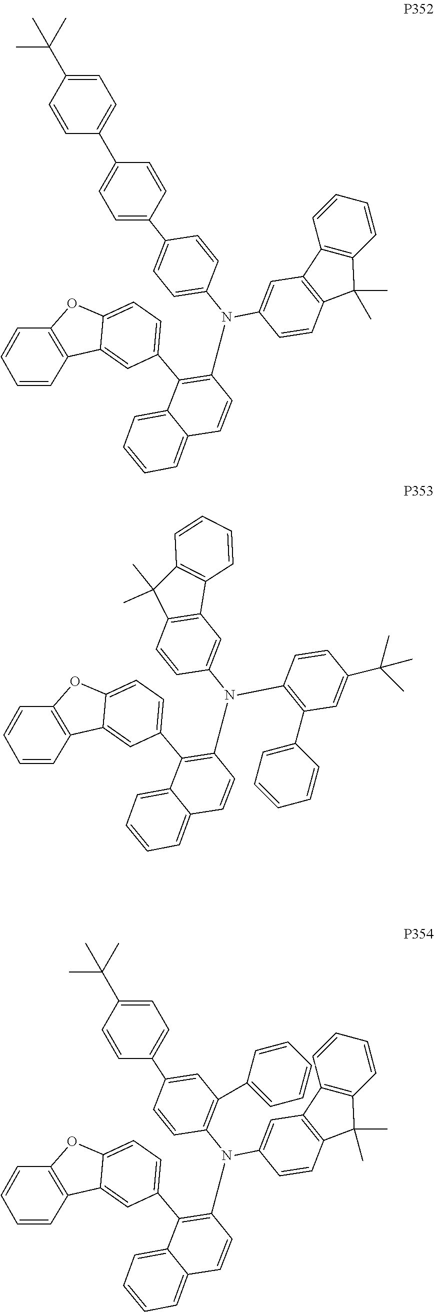 [SMILES: CC(C)(C)c1ccc(-c2ccc(-c3ccc(N(c4ccc5c(c4)-c4ccccc4C5(C)C)c4ccc5ccccc5c4-c4ccc5oc6ccccc6c5c4)cc3)cc2)cc1 ; CC(C)(C)c1ccc(N(c2ccc3c(c2)-c2ccccc2C3(C)C)c2ccc3ccccc3c2-c2ccc3oc4ccccc4c3c2)c(-c2ccccc2)c1 ; CC(C)(C)c1ccc(-c2ccc(N(c3ccc4c(c3)-c3ccccc3C4(C)C)c3ccc4ccccc4c3-c3ccc4oc5ccccc5c4c3)c(-c3ccccc3)c2)cc1]

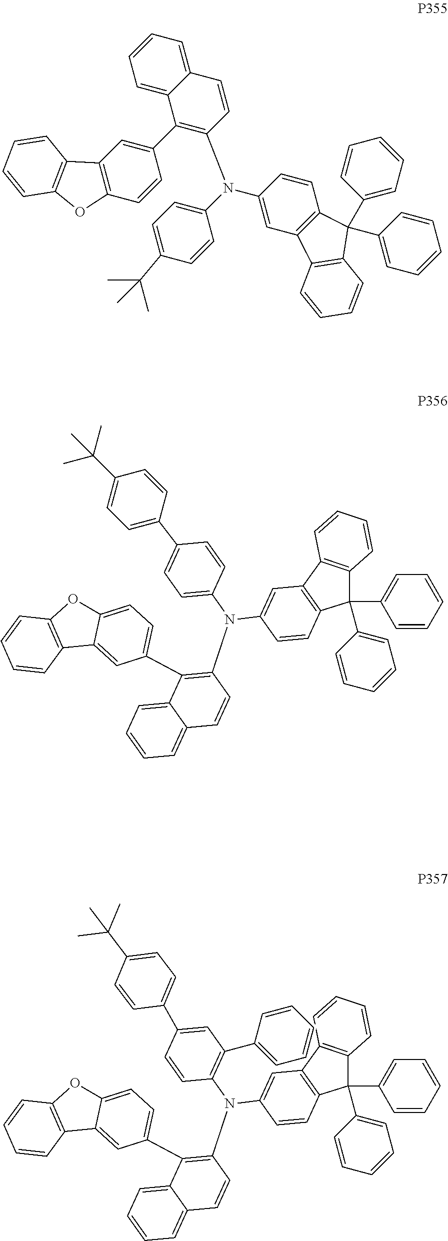 [SMILES: CC(C)(C)c1ccc(N(c2ccc3c(c2)-c2ccccc2C3(c2ccccc2)c2ccccc2)c2ccc3ccccc3c2-c2ccc3oc4ccccc4c3c2)cc1 ; CC(C)(C)c1ccc(-c2ccc(N(c3ccc4c(c3)-c3ccccc3C4(c3ccccc3)c3ccccc3)c3ccc4ccccc4c3-c3ccc4oc5ccccc5c4c3)cc2)cc1 ; CC(C)(C)c1ccc(-c2ccc(N(c3ccc4c(c3)-c3ccccc3C4(c3ccccc3)c3ccccc3)c3ccc4ccccc4c3-c3ccc4oc5ccccc5c4c3)c(-c3ccccc3)c2)cc1]

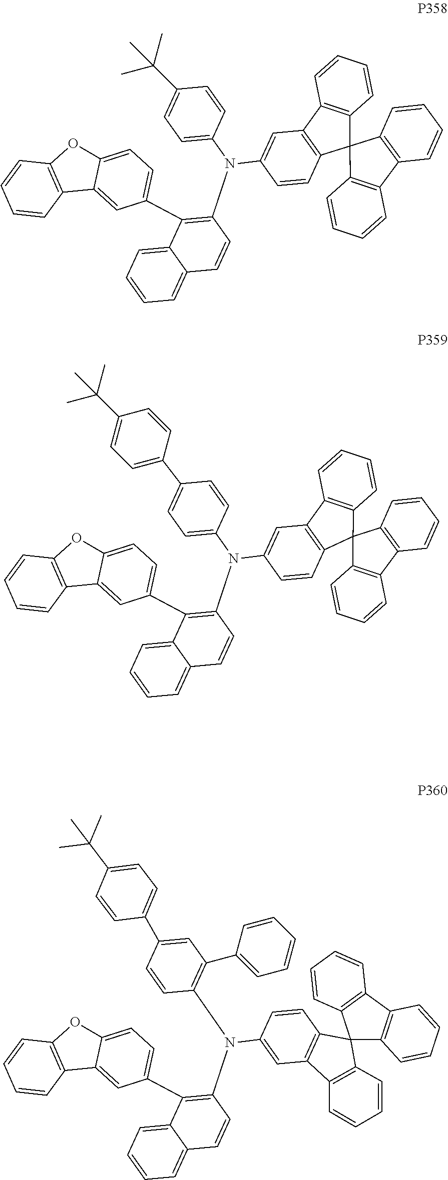 [SMILES: CC(C)(C)c1ccc(N(c2ccc3c(c2)-c2ccccc2C32c3ccccc3-c3ccccc32)c2ccc3ccccc3c2-c2ccc3oc4ccccc4c3c2)cc1 ; CC(C)(C)c1ccc(-c2ccc(N(c3ccc4c(c3)-c3ccccc3C43c4ccccc4-c4ccccc43)c3ccc4ccccc4c3-c3ccc4oc5ccccc5c4c3)cc2)cc1 ; CC(C)(C)c1ccc(-c2ccc(N(c3ccc4c(c3)-c3ccccc3C43c4ccccc4-c4ccccc43)c3ccc4ccccc4c3-c3ccc4oc5ccccc5c4c3)c(-c3ccccc3)c2)cc1]

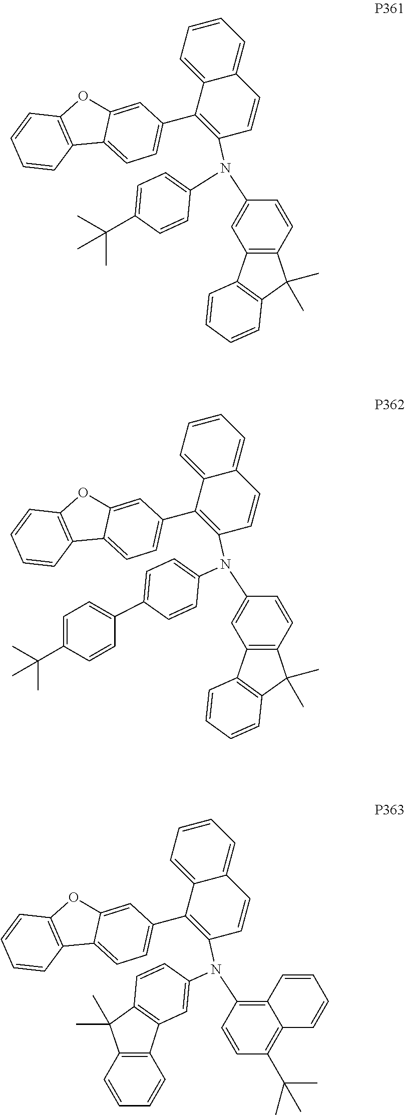 [SMILES: CC(C)(C)c1ccc(N(c2ccc3c(c2)-c2ccccc2C3(C)C)c2ccc3ccccc3c2-c2ccc3c(c2)oc2ccccc23)cc1 ; CC(C)(C)c1ccc(-c2ccc(N(c3ccc4c(c3)-c3ccccc3C4(C)C)c3ccc4ccccc4c3-c3ccc4c(c3)oc3ccccc34)cc2)cc1 ; CC(C)(C)c1ccc(N(c2ccc3c(c2)-c2ccccc2C3(C)C)c2ccc3ccccc3c2-c2ccc3c(c2)oc2ccccc23)c2ccccc12]

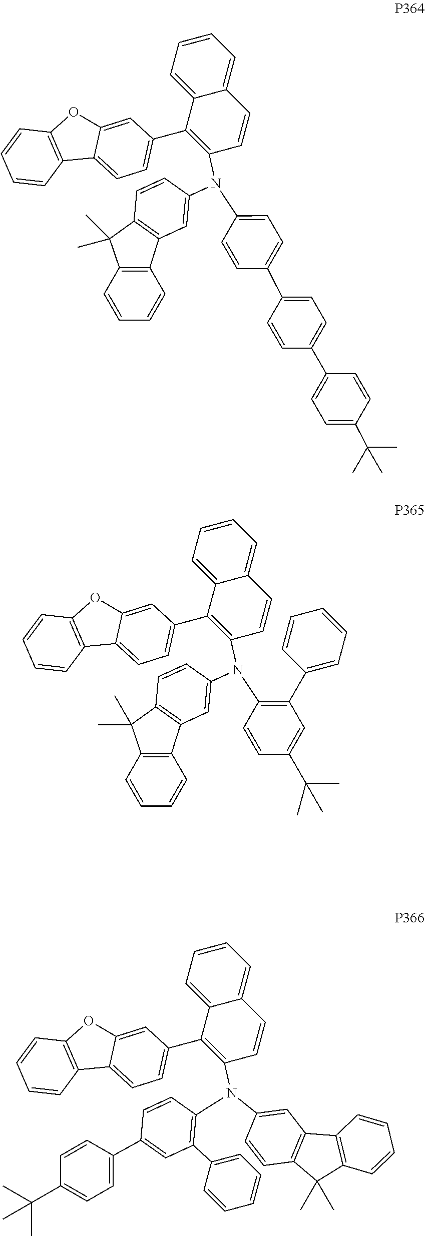

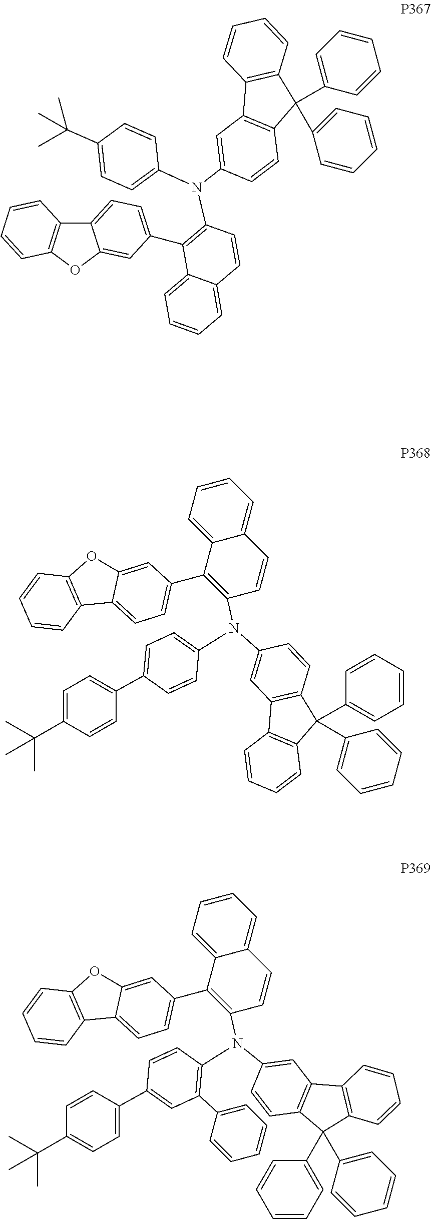 [SMILES: CC(C)(C)c1ccc(N(c2ccc3c(c2)-c2ccccc2C3(c2ccccc2)c2ccccc2)c2ccc3ccccc3c2-c2ccc3c(c2)oc2ccccc23)cc1 ; CC(C)(C)c1ccc(-c2ccc(N(c3ccc4c(c3)-c3ccccc3C4(c3ccccc3)c3ccccc3)c3ccc4ccccc4c3-c3ccc4c(c3)oc3ccccc34)cc2)cc1 ; CC(C)(C)c1ccc(-c2ccc(N(c3ccc4c(c3)-c3ccccc3C4(c3ccccc3)c3ccccc3)c3ccc4ccccc4c3-c3ccc4c(c3)oc3ccccc34)c(-c3ccccc3)c2)cc1]

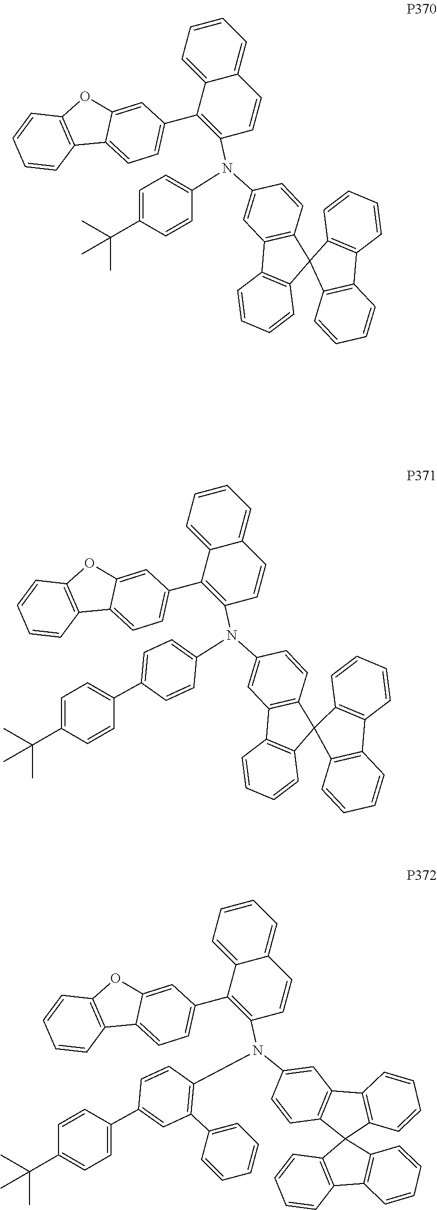 [SMILES: CC(C)(C)c1ccc(N(c2ccc3c(c2)-c2ccccc2C32c3ccccc3-c3ccccc32)c2ccc3ccccc3c2-c2ccc3c(c2)oc2ccccc23)cc1 ; CC(C)(C)c1ccc(-c2ccc(N(c3ccc4c(c3)-c3ccccc3C43c4ccccc4-c4ccccc43)c3ccc4ccccc4c3-c3ccc4c(c3)oc3ccccc34)cc2)cc1 ; CC(C)(C)c1ccc(-c2ccc(N(c3ccc4c(c3)-c3ccccc3C43c4ccccc4-c4ccccc43)c3ccc4ccccc4c3-c3ccc4c(c3)oc3ccccc34)c(-c3ccccc3)c2)cc1]

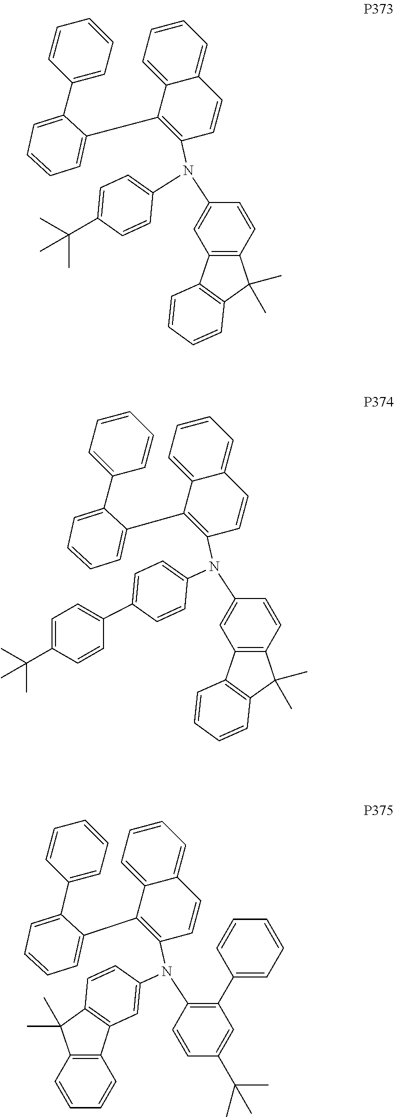

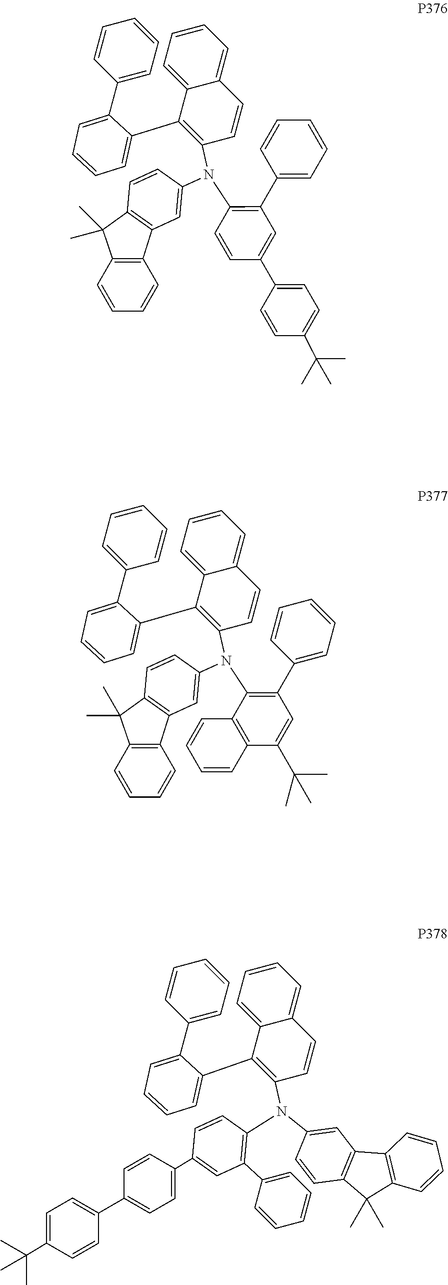

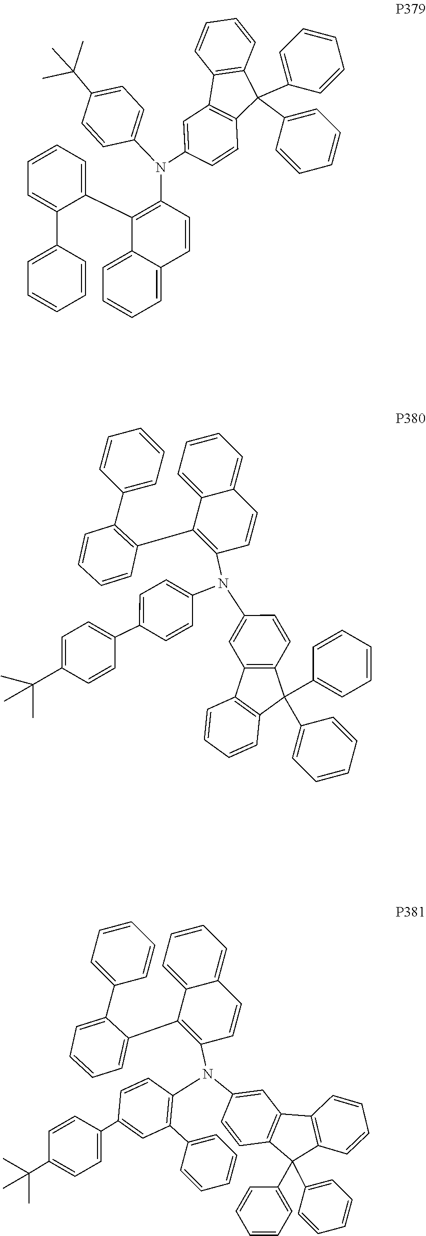 [SMILES: CC(C)(C)c1ccc(N(c2ccc3c(c2)-c2ccccc2C3(c2ccccc2)c2ccccc2)c2ccc3ccccc3c2-c2ccccc2-c2ccccc2)cc1 ; CC(C)(C)c1ccc(-c2ccc(N(c3ccc4c(c3)-c3ccccc3C4(c3ccccc3)c3ccccc3)c3ccc4ccccc4c3-c3ccccc3-c3ccccc3)cc2)cc1 ; CC(C)(C)c1ccc(-c2ccc(N(c3ccc4c(c3)-c3ccccc3C4(c3ccccc3)c3ccccc3)c3ccc4ccccc4c3-c3ccccc3-c3ccccc3)c(-c3ccccc3)c2)cc1]

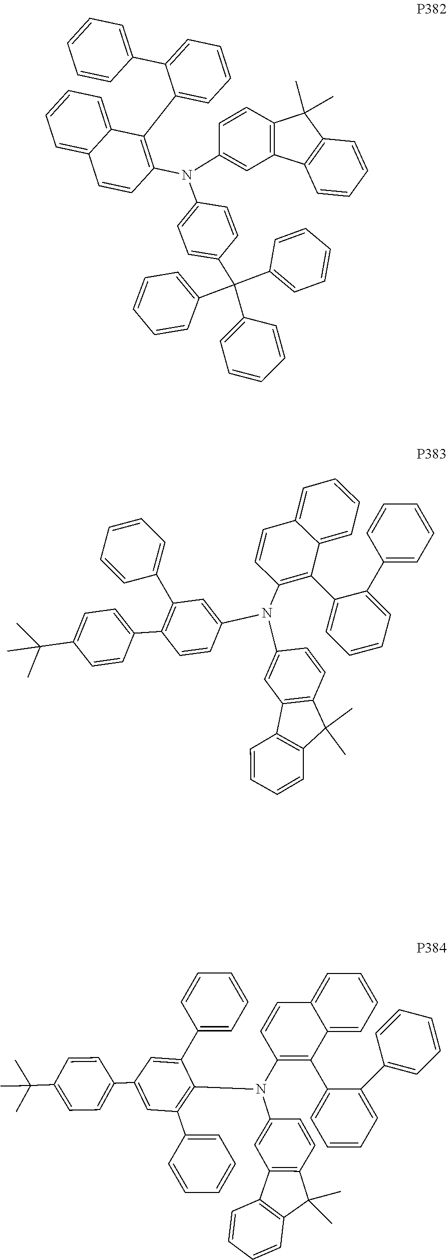 [SMILES: CC1(C)c2ccccc2-c2cc(N(c3ccc(C(c4ccccc4)(c4ccccc4)c4ccccc4)cc3)c3ccc4ccccc4c3-c3ccccc3-c3ccccc3)ccc21 ; CC(C)(C)c1ccc(-c2ccc(N(c3ccc4c(c3)-c3ccccc3C4(C)C)c3ccc4ccccc4c3-c3ccccc3-c3ccccc3)cc2-c2ccccc2)cc1 ; CC(C)(C)c1ccc(-c2cc(-c3ccccc3)c(N(c3ccc4c(c3)-c3ccccc3C4(C)C)c3ccc4ccccc4c3-c3ccccc3-c3ccccc3)c(-c3ccccc3)c2)cc1]

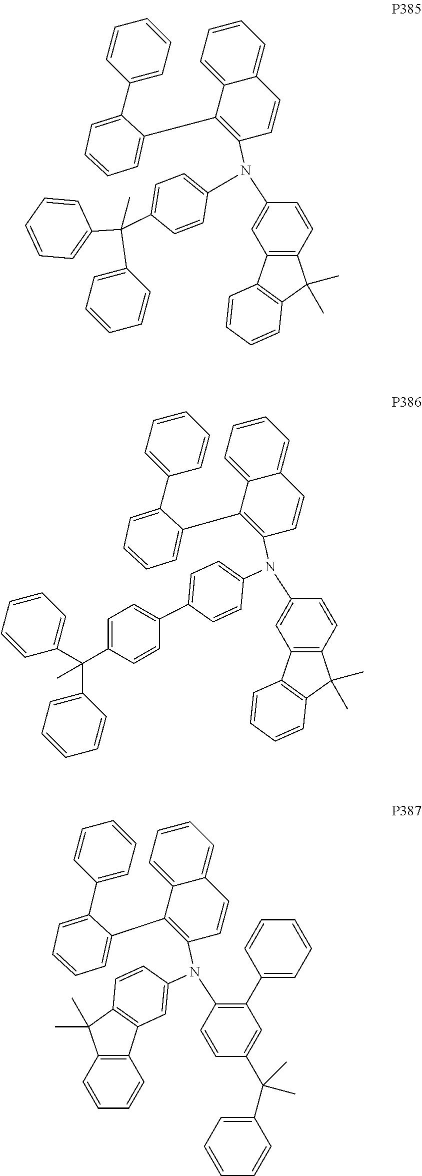 [SMILES: CC1(C)c2ccccc2-c2cc(N(c3ccc(C(C)(c4ccccc4)c4ccccc4)cc3)c3ccc4ccccc4c3-c3ccccc3-c3ccccc3)ccc21 ; CC1(C)c2ccccc2-c2cc(N(c3ccc(-c4ccc(C(C)(c5ccccc5)c5ccccc5)cc4)cc3)c3ccc4ccccc4c3-c3ccccc3-c3ccccc3)ccc21 ; CC(C)(c1ccccc1)c1ccc(N(c2ccc3c(c2)-c2ccccc2C3(C)C)c2ccc3ccccc3c2-c2ccccc2-c2ccccc2)c(-c2ccccc2)c1]

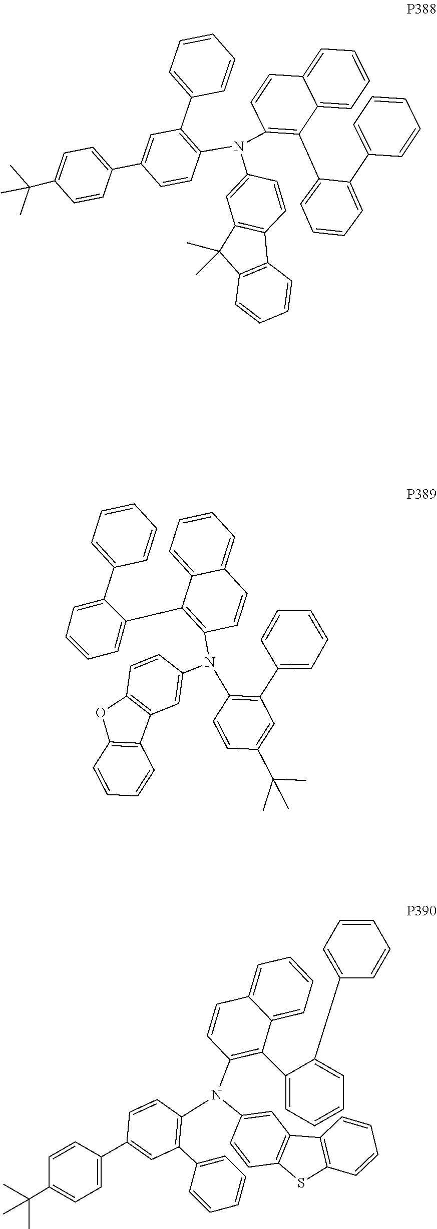 [SMILES: CC(C)(C)c1ccc(-c2ccc(N(c3ccc4c(c3)C(C)(C)c3ccccc3-4)c3ccc4ccccc4c3-c3ccccc3-c3ccccc3)c(-c3ccccc3)c2)cc1 ; CC(C)(C)c1ccc(N(c2ccc3oc4ccccc4c3c2)c2ccc3ccccc3c2-c2ccccc2-c2ccccc2)c(-c2ccccc2)c1 ; CC(C)(C)c1ccc(-c2ccc(N(c3ccc4sc5ccccc5c4c3)c3ccc4ccccc4c3-c3ccccc3-c3ccccc3)c(-c3ccccc3)c2)cc1]

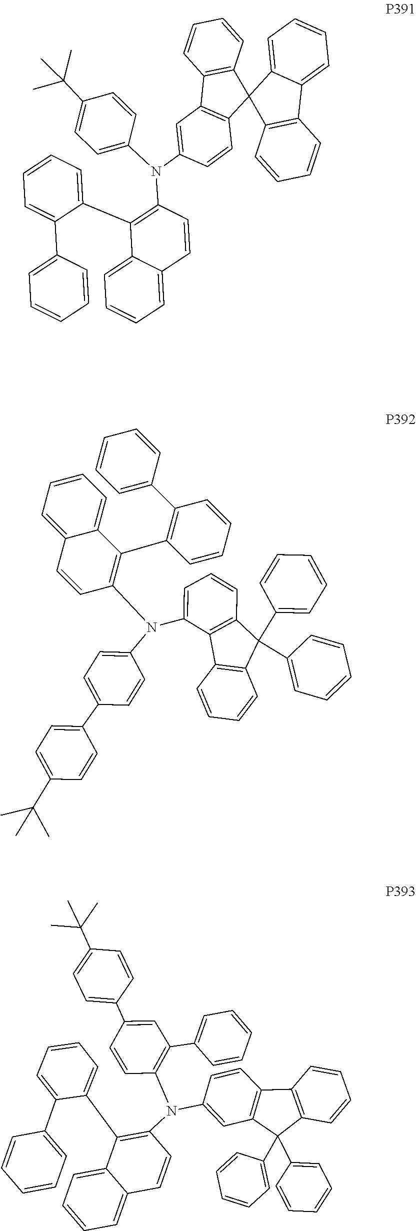 [SMILES: CC(C)(C)c1ccc(N(c2ccc3c(c2)-c2ccccc2C32c3ccccc3-c3ccccc32)c2ccc3ccccc3c2-c2ccccc2-c2ccccc2)cc1 ; CC(C)(C)c1ccc(-c2ccc(N(c3cccc4c3-c3ccccc3C4(c3ccccc3)c3ccccc3)c3ccc4ccccc4c3-c3ccccc3-c3ccccc3)cc2)cc1 ; CC(C)(C)c1ccc(-c2ccc(N(c3ccc4c(c3)C(c3ccccc3)(c3ccccc3)c3ccccc3-4)c3ccc4ccccc4c3-c3ccccc3-c3ccccc3)c(-c3ccccc3)c2)cc1]

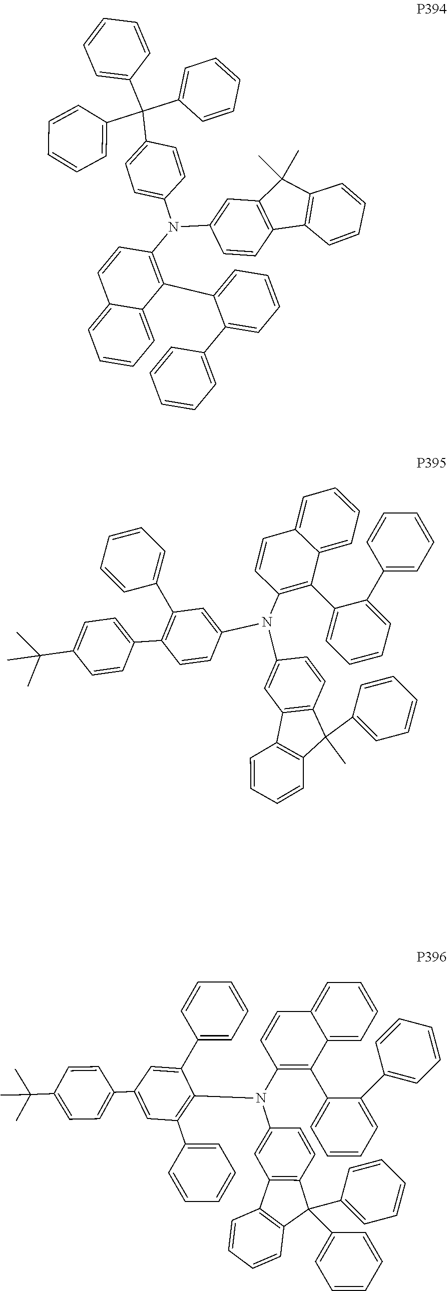 [SMILES: CC1(C)c2ccccc2-c2ccc(N(c3ccc(C(c4ccccc4)(c4ccccc4)c4ccccc4)cc3)c3ccc4ccccc4c3-c3ccccc3-c3ccccc3)cc21 ; CC(C)(C)c1ccc(-c2ccc(N(c3ccc4c(c3)-c3ccccc3C4(C)c3ccccc3)c3ccc4ccccc4c3-c3ccccc3-c3ccccc3)cc2-c2ccccc2)cc1 ; CC(C)(C)c1ccc(-c2cc(-c3ccccc3)c(N(c3ccc4c(c3)-c3ccccc3C4(c3ccccc3)c3ccccc3)c3ccc4ccccc4c3-c3ccccc3-c3ccccc3)c(-c3ccccc3)c2)cc1]

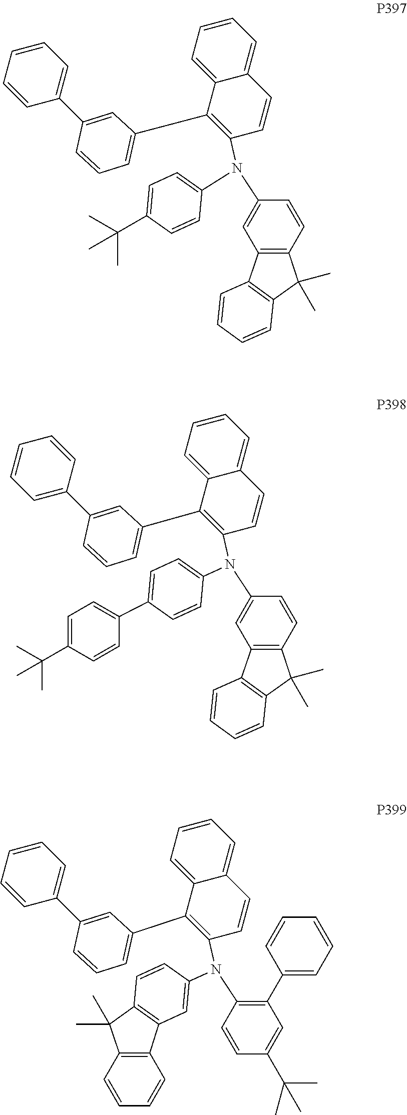 [SMILES: CC(C)(C)c1ccc(N(c2ccc3c(c2)-c2ccccc2C3(C)C)c2ccc3ccccc3c2-c2cccc(-c3ccccc3)c2)cc1 ; CC(C)(C)c1ccc(-c2ccc(N(c3ccc4c(c3)-c3ccccc3C4(C)C)c3ccc4ccccc4c3-c3cccc(-c4ccccc4)c3)cc2)cc1 ; CC(C)(C)c1ccc(N(c2ccc3c(c2)-c2ccccc2C3(C)C)c2ccc3ccccc3c2-c2cccc(-c3ccccc3)c2)c(-c2ccccc2)c1]

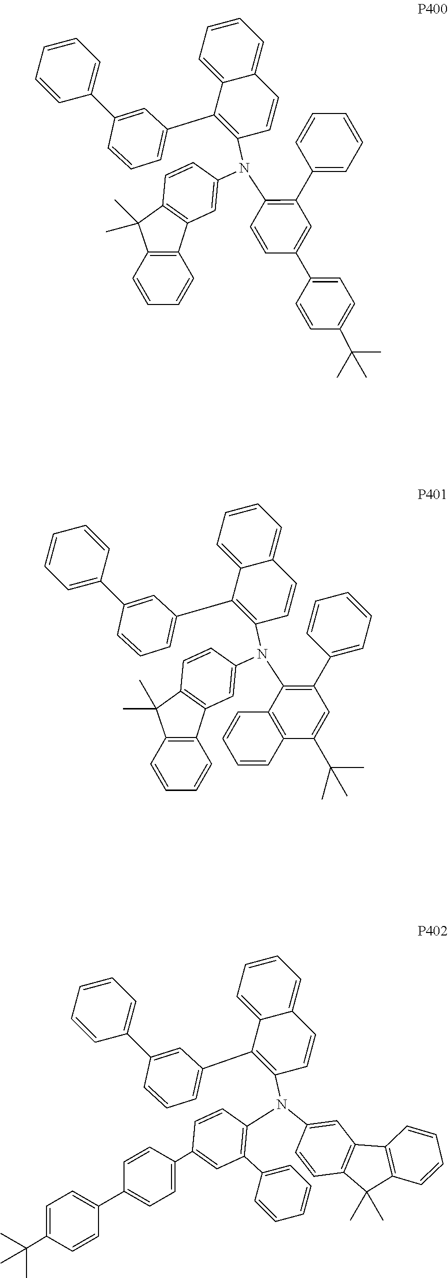

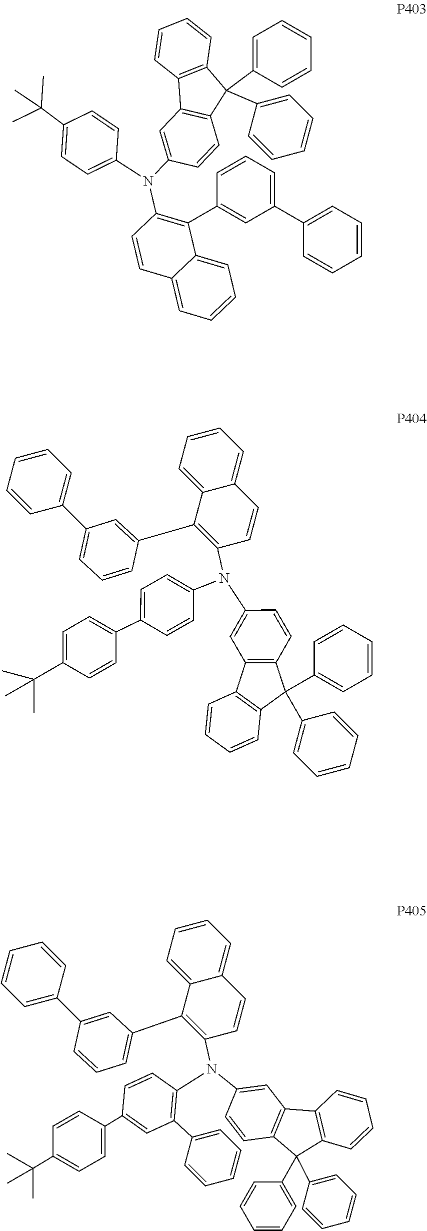 [SMILES: CC(C)(C)c1ccc(N(c2ccc3c(c2)-c2ccccc2C3(c2ccccc2)c2ccccc2)c2ccc3ccccc3c2-c2cccc(-c3ccccc3)c2)cc1 ; CC(C)(C)c1ccc(-c2ccc(N(c3ccc4c(c3)-c3ccccc3C4(c3ccccc3)c3ccccc3)c3ccc4ccccc4c3-c3cccc(-c4ccccc4)c3)cc2)cc1 ; CC(C)(C)c1ccc(-c2ccc(N(c3ccc4c(c3)-c3ccccc3C4(c3ccccc3)c3ccccc3)c3ccc4ccccc4c3-c3cccc(-c4ccccc4)c3)c(-c3ccccc3)c2)cc1]

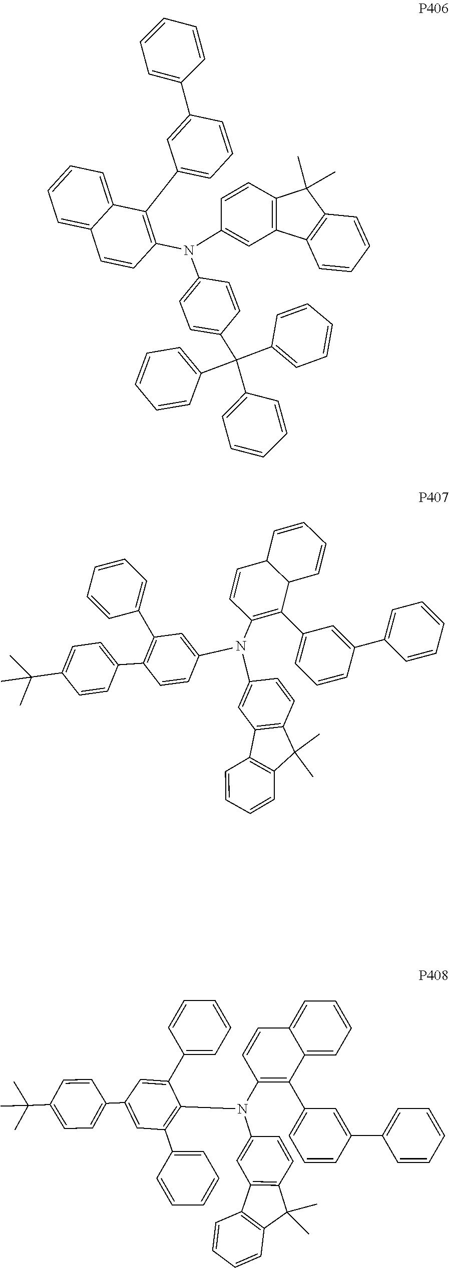 [SMILES: CC1(C)c2ccccc2-c2cc(N(c3ccc(C(c4ccccc4)(c4ccccc4)c4ccccc4)cc3)c3ccc4ccccc4c3-c3cccc(-c4ccccc4)c3)ccc21 ; CC(C)(C)c1ccc(-c2ccc(N(C3=C(c4cccc(-c5ccccc5)c4)C4C=CC=CC4C=C3)c3ccc4c(c3)-c3ccccc3C4(C)C)cc2-c2ccccc2)cc1 ; CC(C)(C)c1ccc(-c2cc(-c3ccccc3)c(N(c3ccc4c(c3)-c3ccccc3C4(C)C)c3ccc4ccccc4c3-c3cccc(-c4ccccc4)c3)c(-c3ccccc3)c2)cc1]

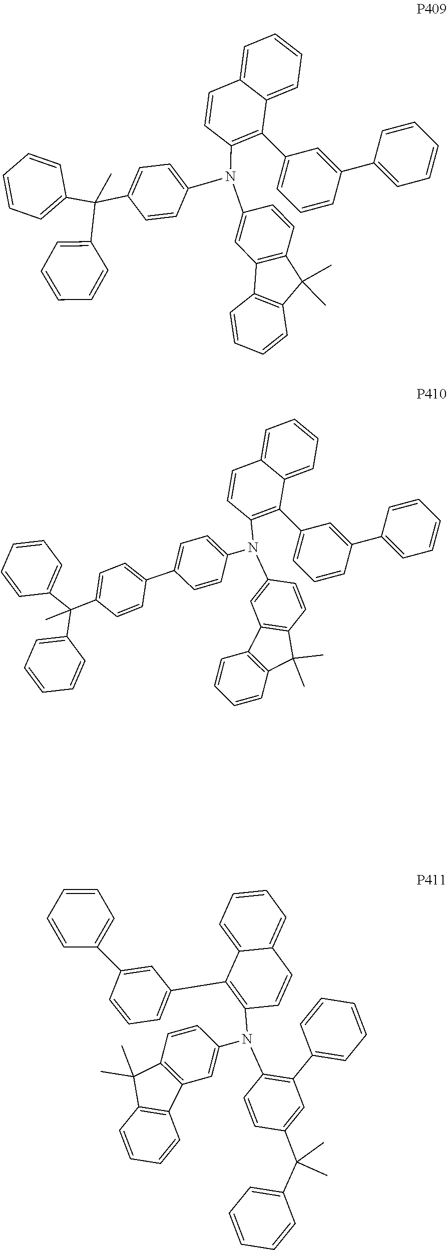 [SMILES: CC1(C)c2ccccc2-c2cc(N(c3ccc(C(C)(c4ccccc4)c4ccccc4)cc3)c3ccc4ccccc4c3-c3cccc(-c4ccccc4)c3)ccc21 ; CC1(C)c2ccccc2-c2cc(N(c3ccc(-c4ccc(C(C)(c5ccccc5)c5ccccc5)cc4)cc3)c3ccc4ccccc4c3-c3cccc(-c4ccccc4)c3)ccc21 ; CC(C)(c1ccccc1)c1ccc(N(c2ccc3c(c2)-c2ccccc2C3(C)C)c2ccc3ccccc3c2-c2cccc(-c3ccccc3)c2)c(-c2ccccc2)c1]

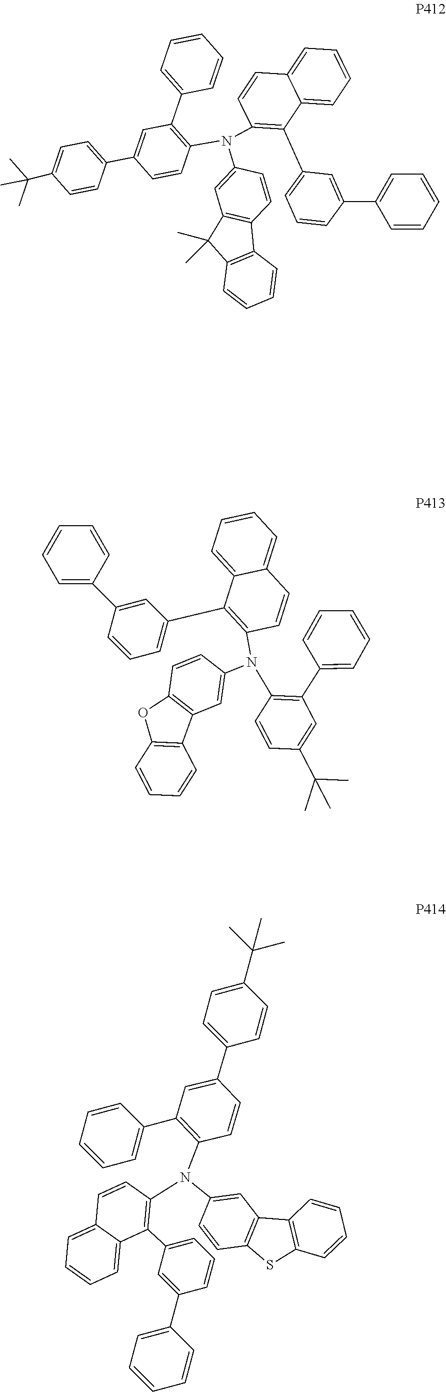 [SMILES: CC(C)(C)c1ccc(-c2ccc(N(c3ccc4c(c3)C(C)(C)c3ccccc3-4)c3ccc4ccccc4c3-c3cccc(-c4ccccc4)c3)c(-c3ccccc3)c2)cc1 ; CC(C)(C)c1ccc(N(c2ccc3oc4ccccc4c3c2)c2ccc3ccccc3c2-c2cccc(-c3ccccc3)c2)c(-c2ccccc2)c1 ; CC(C)(C)c1ccc(-c2ccc(N(c3ccc4sc5ccccc5c4c3)c3ccc4ccccc4c3-c3cccc(-c4ccccc4)c3)c(-c3ccccc3)c2)cc1]

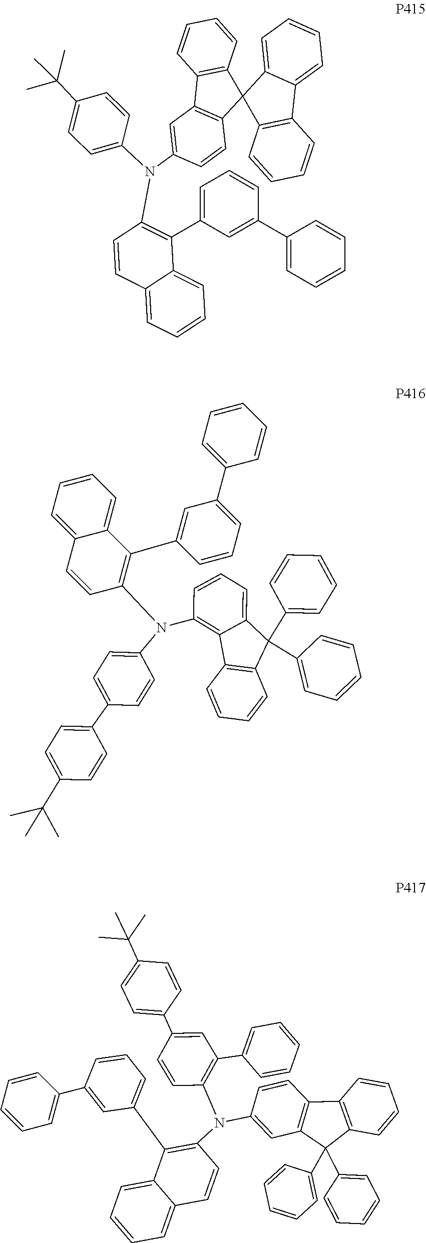 [SMILES: CC(C)(C)c1ccc(N(c2ccc3c(c2)-c2ccccc2C32c3ccccc3-c3ccccc32)c2ccc3ccccc3c2-c2cccc(-c3ccccc3)c2)cc1 ; CC(C)(C)c1ccc(-c2ccc(N(c3cccc4c3-c3ccccc3C4(c3ccccc3)c3ccccc3)c3ccc4ccccc4c3-c3cccc(-c4ccccc4)c3)cc2)cc1 ; CC(C)(C)c1ccc(-c2ccc(N(c3ccc4c(c3)C(c3ccccc3)(c3ccccc3)c3ccccc3-4)c3ccc4ccccc4c3-c3cccc(-c4ccccc4)c3)c(-c3ccccc3)c2)cc1]

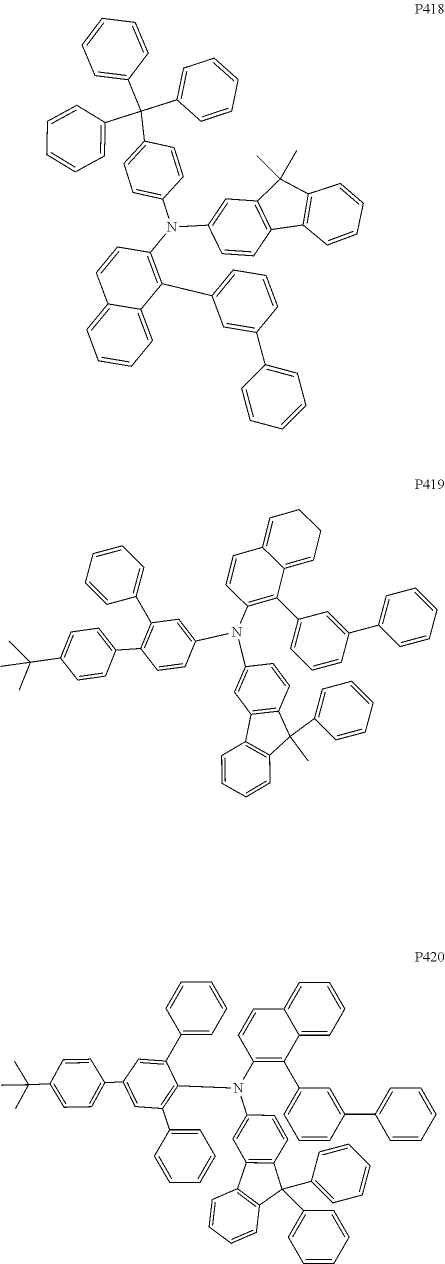 [SMILES: CC1(C)c2ccccc2-c2ccc(N(c3ccc(C(c4ccccc4)(c4ccccc4)c4ccccc4)cc3)c3ccc4ccccc4c3-c3cccc(-c4ccccc4)c3)cc21 ; CC(C)(C)c1ccc(-c2ccc(N(c3ccc4c(c3)-c3ccccc3C4(C)c3ccccc3)c3ccc4c(c3-c3cccc(-c5ccccc5)c3)=CCCC=4)cc2-c2ccccc2)cc1 ; CC(C)(C)c1ccc(-c2cc(-c3ccccc3)c(N(c3ccc4c(c3)-c3ccccc3C4(c3ccccc3)c3ccccc3)c3ccc4ccccc4c3-c3cccc(-c4ccccc4)c3)c(-c3ccccc3)c2)cc1]

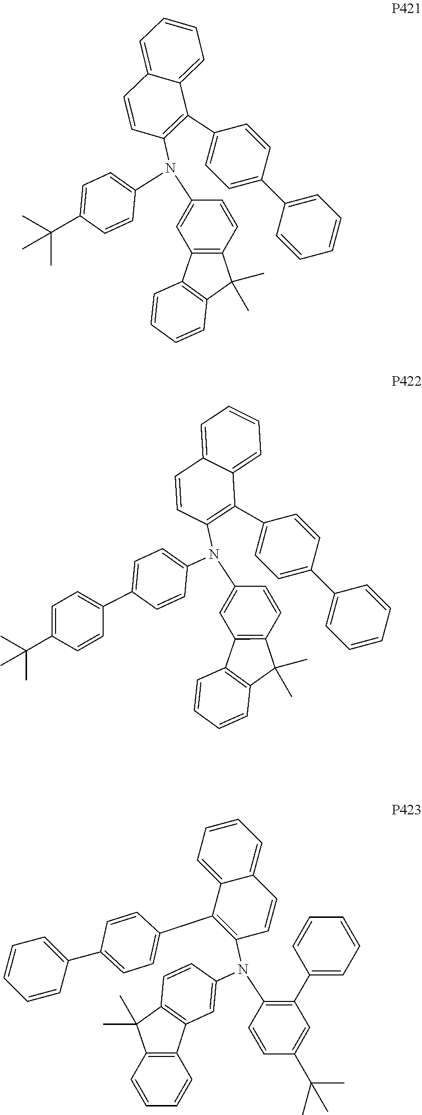 [SMILES: CC(C)(C)c1ccc(N(c2ccc3c(c2)-c2ccccc2C3(C)C)c2ccc3ccccc3c2-c2ccc(-c3ccccc3)cc2)cc1 ; CC(C)(C)c1ccc(-c2ccc(N(c3ccc4c(c3)-c3ccccc3C4(C)C)c3ccc4ccccc4c3-c3ccc(-c4ccccc4)cc3)cc2)cc1 ; CC(C)(C)c1ccc(N(c2ccc3c(c2)-c2ccccc2C3(C)C)c2ccc3ccccc3c2-c2ccc(-c3ccccc3)cc2)c(-c2ccccc2)c1]

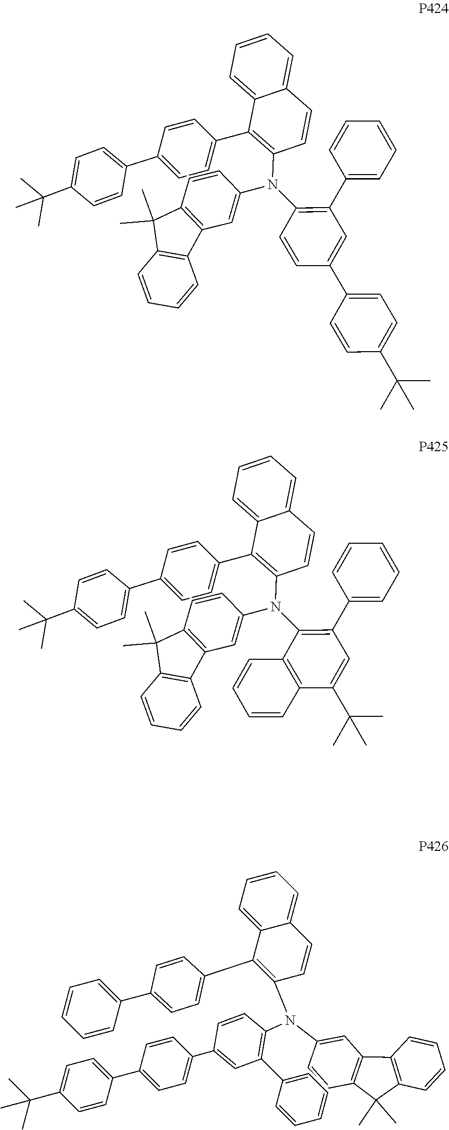

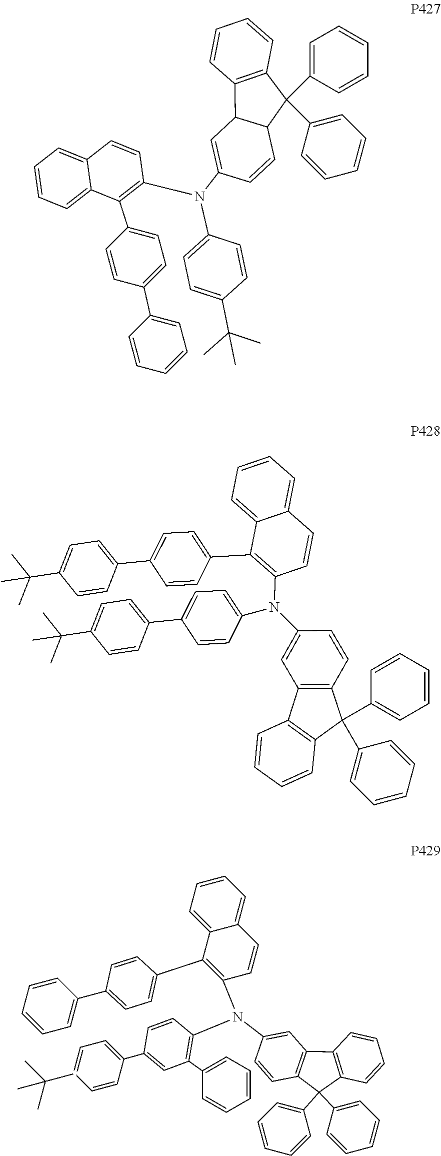

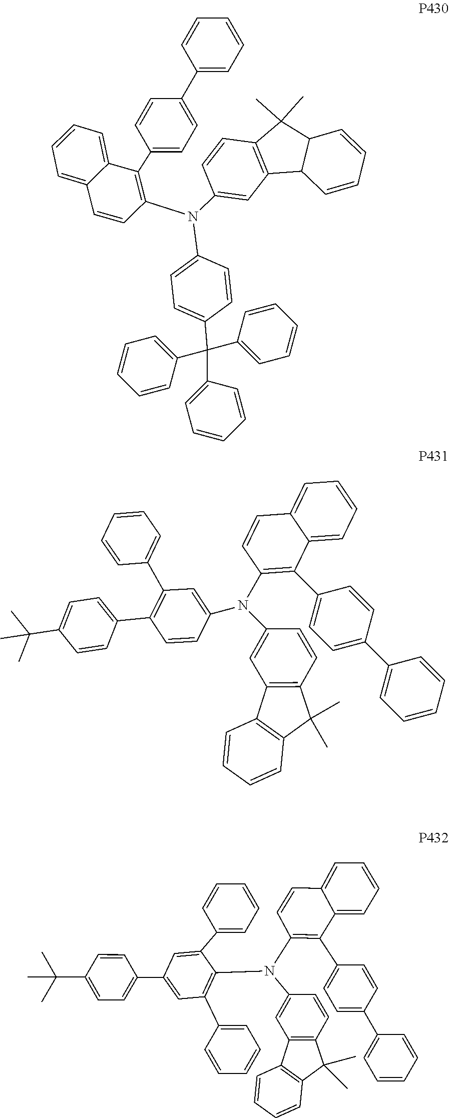

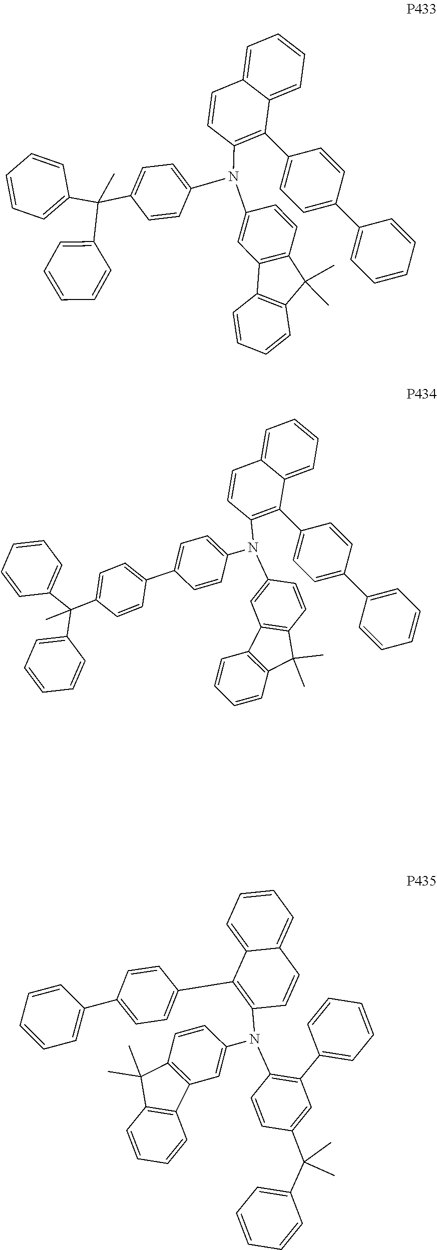 [SMILES: CC1(C)c2ccccc2-c2cc(N(c3ccc(C(C)(c4ccccc4)c4ccccc4)cc3)c3ccc4ccccc4c3-c3ccc(-c4ccccc4)cc3)ccc21 ; CC1(C)c2ccccc2-c2cc(N(c3ccc(-c4ccc(C(C)(c5ccccc5)c5ccccc5)cc4)cc3)c3ccc4ccccc4c3-c3ccc(-c4ccccc4)cc3)ccc21 ; CC(C)(c1ccccc1)c1ccc(N(c2ccc3c(c2)-c2ccccc2C3(C)C)c2ccc3ccccc3c2-c2ccc(-c3ccccc3)cc2)c(-c2ccccc2)c1]

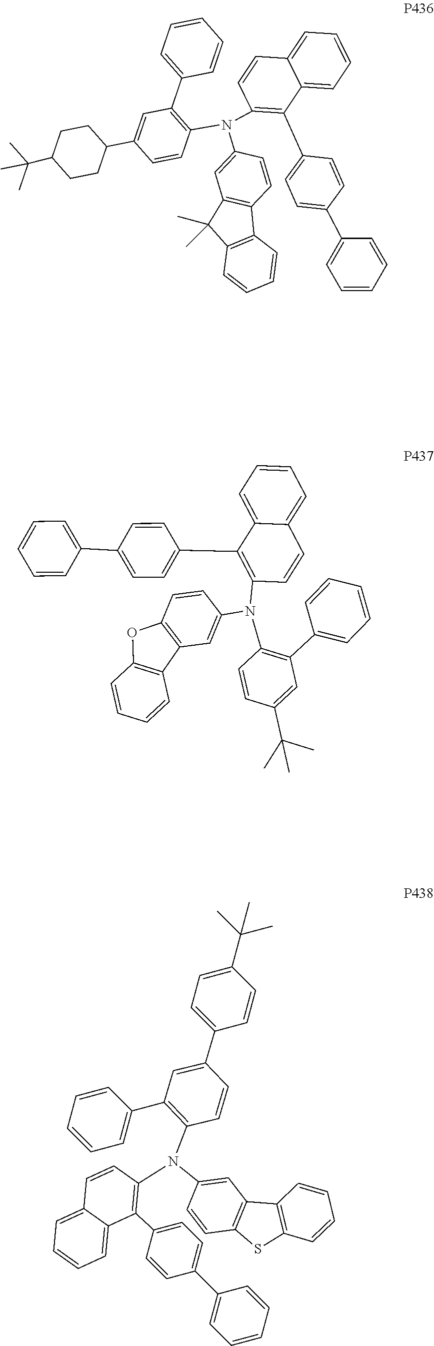 [SMILES: CC1(C)c2ccccc2-c2ccc(N(c3ccc(C4CCC(C(C)(C)C)CC4)cc3-c3ccccc3)c3ccc4ccccc4c3-c3ccc(-c4ccccc4)cc3)cc21 ; CC(C)(C)c1ccc(N(c2ccc3oc4ccccc4c3c2)c2ccc3ccccc3c2-c2ccc(-c3ccccc3)cc2)c(-c2ccccc2)c1 ; CC(C)(C)c1ccc(-c2ccc(N(c3ccc4sc5ccccc5c4c3)c3ccc4ccccc4c3-c3ccc(-c4ccccc4)cc3)c(-c3ccccc3)c2)cc1]

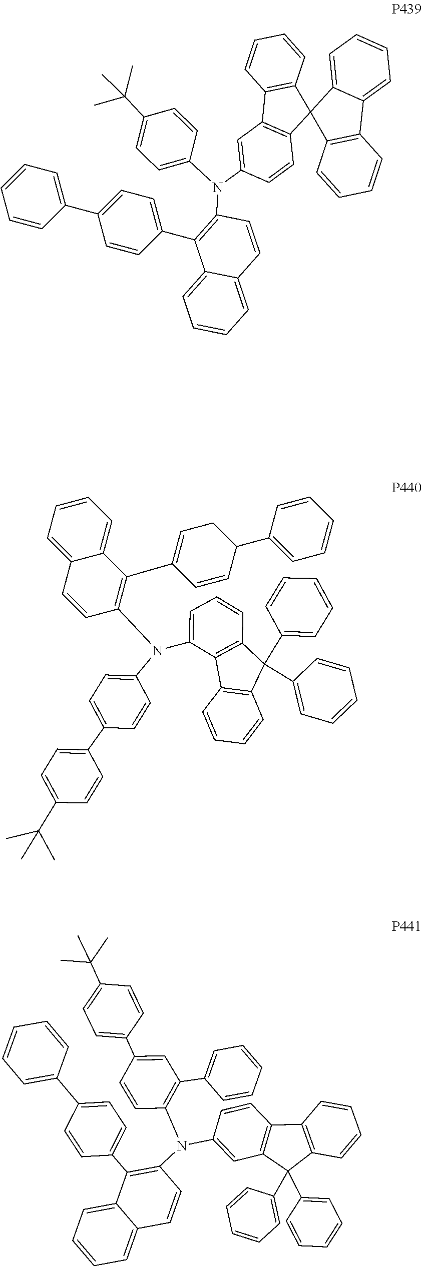 [SMILES: CC(C)(C)c1ccc(N(c2ccc3c(c2)-c2ccccc2C32c3ccccc3-c3ccccc32)c2ccc3ccccc3c2-c2ccc(-c3ccccc3)cc2)cc1 ; CC(C)(C)c1ccc(-c2ccc(N(c3cccc4c3-c3ccccc3C4(c3ccccc3)c3ccccc3)c3ccc4ccccc4c3C3=CCC(c4ccccc4)C=C3)cc2)cc1 ; CC(C)(C)c1ccc(-c2ccc(N(c3ccc4c(c3)C(c3ccccc3)(c3ccccc3)c3ccccc3-4)c3ccc4ccccc4c3-c3ccc(-c4ccccc4)cc3)c(-c3ccccc3)c2)cc1]

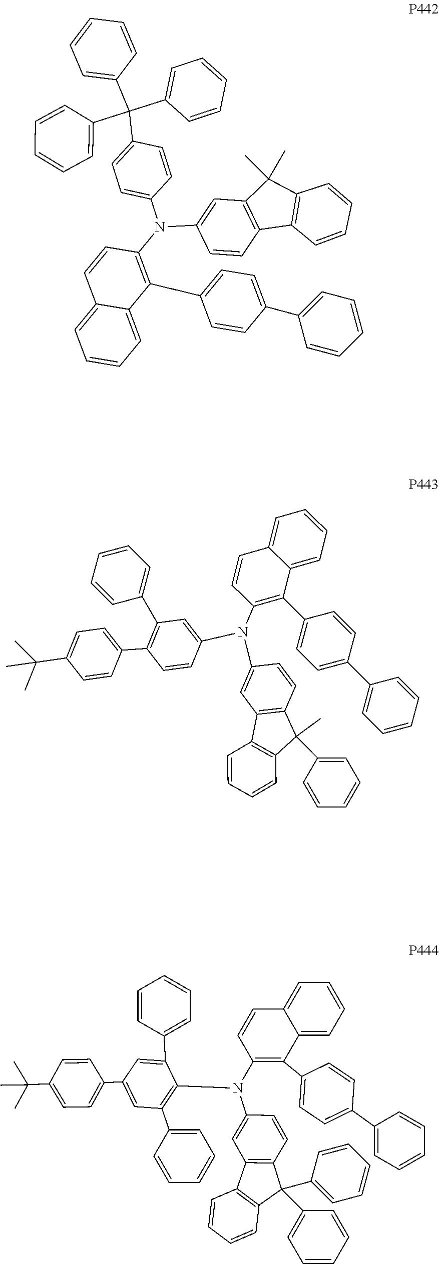 [SMILES: CC1(C)c2ccccc2-c2ccc(N(c3ccc(C(c4ccccc4)(c4ccccc4)c4ccccc4)cc3)c3ccc4ccccc4c3-c3ccc(-c4ccccc4)cc3)cc21 ; CC(C)(C)c1ccc(-c2ccc(N(c3ccc4c(c3)-c3ccccc3C4(C)c3ccccc3)c3ccc4ccccc4c3-c3ccc(-c4ccccc4)cc3)cc2-c2ccccc2)cc1 ; CC(C)(C)c1ccc(-c2cc(-c3ccccc3)c(N(c3ccc4c(c3)-c3ccccc3C4(c3ccccc3)c3ccccc3)c3ccc4ccccc4c3-c3ccc(-c4ccccc4)cc3)c(-c3ccccc3)c2)cc1]

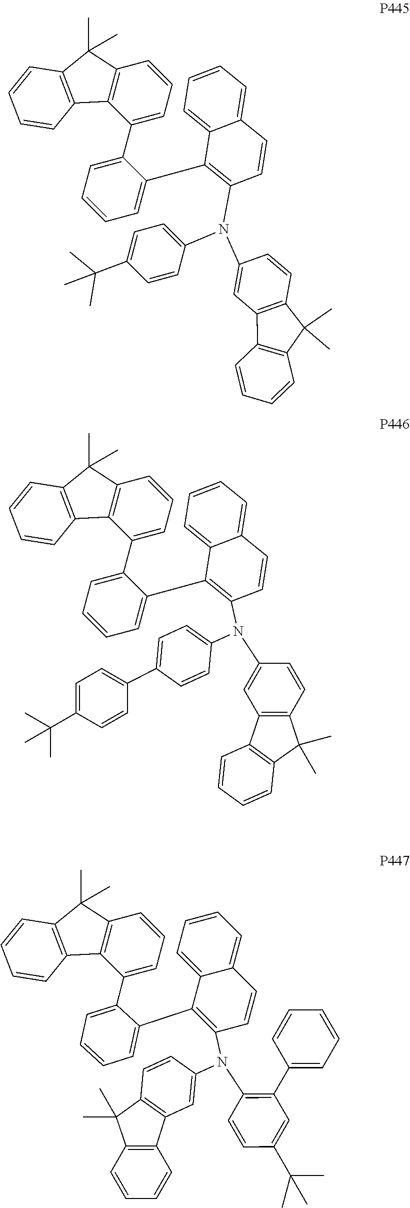 [SMILES: CC(C)(C)c1ccc(N(c2ccc3c(c2)-c2ccccc2C3(C)C)c2ccc3ccccc3c2-c2ccccc2-c2cccc3c2-c2ccccc2C3(C)C)cc1 ; CC(C)(C)c1ccc(-c2ccc(N(c3ccc4c(c3)-c3ccccc3C4(C)C)c3ccc4ccccc4c3-c3ccccc3-c3cccc4c3-c3ccccc3C4(C)C)cc2)cc1 ; CC(C)(C)c1ccc(N(c2ccc3c(c2)-c2ccccc2C3(C)C)c2ccc3ccccc3c2-c2ccccc2-c2cccc3c2-c2ccccc2C3(C)C)c(-c2ccccc2)c1]

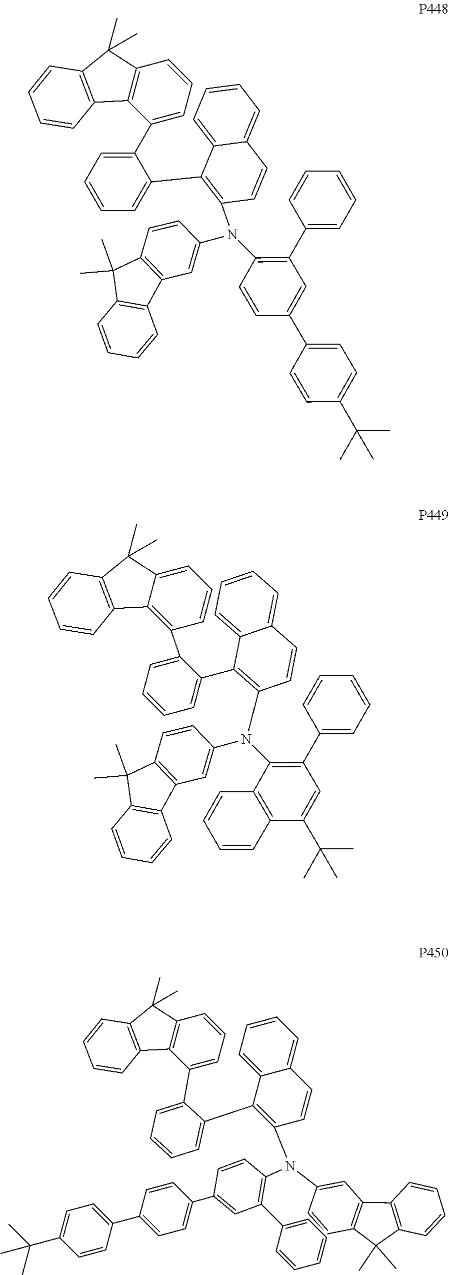

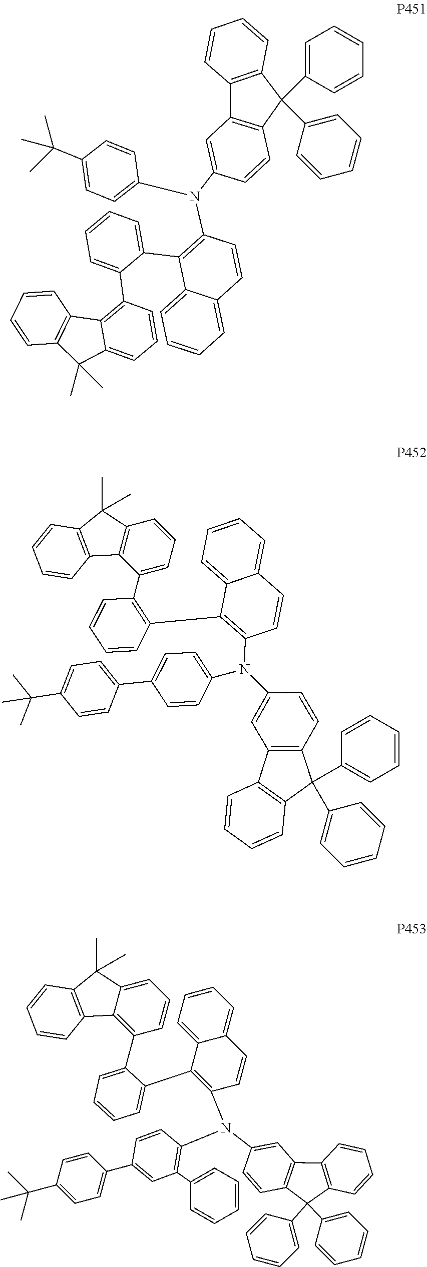

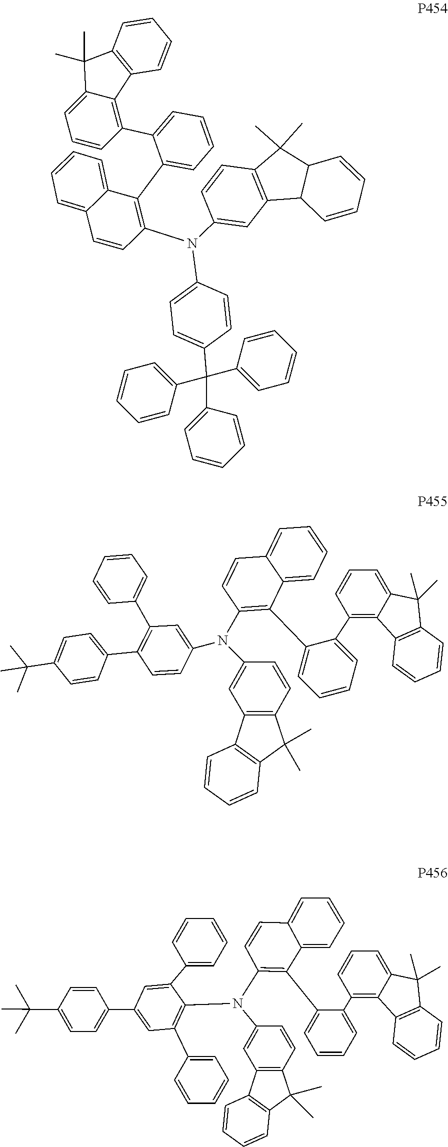

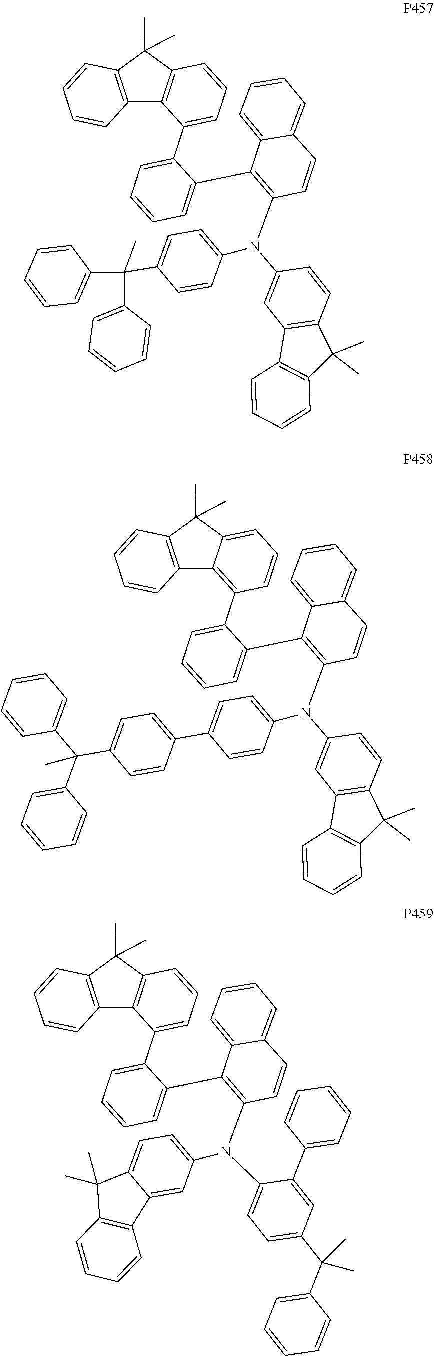 [SMILES: CC1(C)c2ccccc2-c2cc(N(c3ccc(C(C)(c4ccccc4)c4ccccc4)cc3)c3ccc4ccccc4c3-c3ccccc3-c3cccc4c3-c3ccccc3C4(C)C)ccc21 ; CC1(C)c2ccccc2-c2cc(N(c3ccc(-c4ccc(C(C)(c5ccccc5)c5ccccc5)cc4)cc3)c3ccc4ccccc4c3-c3ccccc3-c3cccc4c3-c3ccccc3C4(C)C)ccc21 ; CC(C)(c1ccccc1)c1ccc(N(c2ccc3c(c2)-c2ccccc2C3(C)C)c2ccc3ccccc3c2-c2ccccc2-c2cccc3c2-c2ccccc2C3(C)C)c(-c2ccccc2)c1]

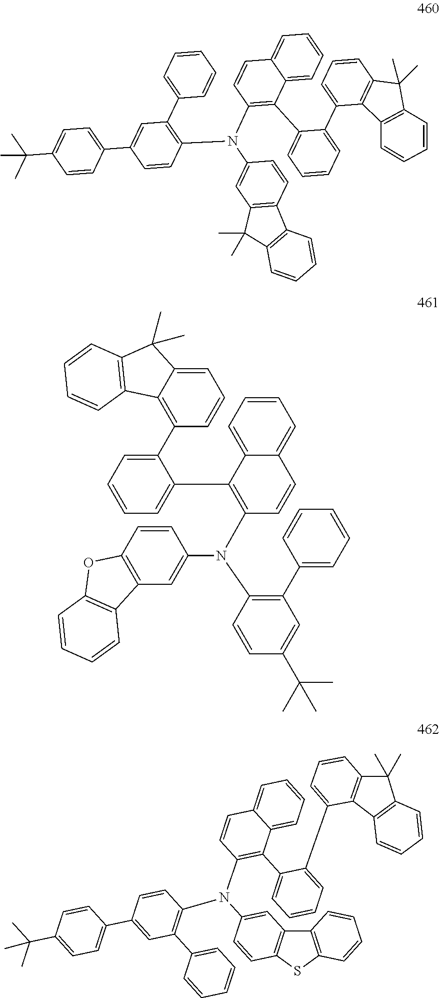 [SMILES: CC(C)(C)c1ccc(-c2ccc(N(c3ccc4c(c3)C(C)(C)c3ccccc3-4)c3ccc4ccccc4c3-c3ccccc3-c3cccc4c3-c3ccccc3C4(C)C)c(-c3ccccc3)c2)cc1 ; CC(C)(C)c1ccc(N(c2ccc3oc4ccccc4c3c2)c2ccc3ccccc3c2-c2ccccc2-c2cccc3c2-c2ccccc2C3(C)C)c(-c2ccccc2)c1 ; CC(C)(C)c1ccc(-c2ccc(N(c3ccc4sc5ccccc5c4c3)c3ccc4ccccc4c3-c3ccccc3-c3cccc4c3-c3ccccc3C4(C)C)c(-c3ccccc3)c2)cc1]

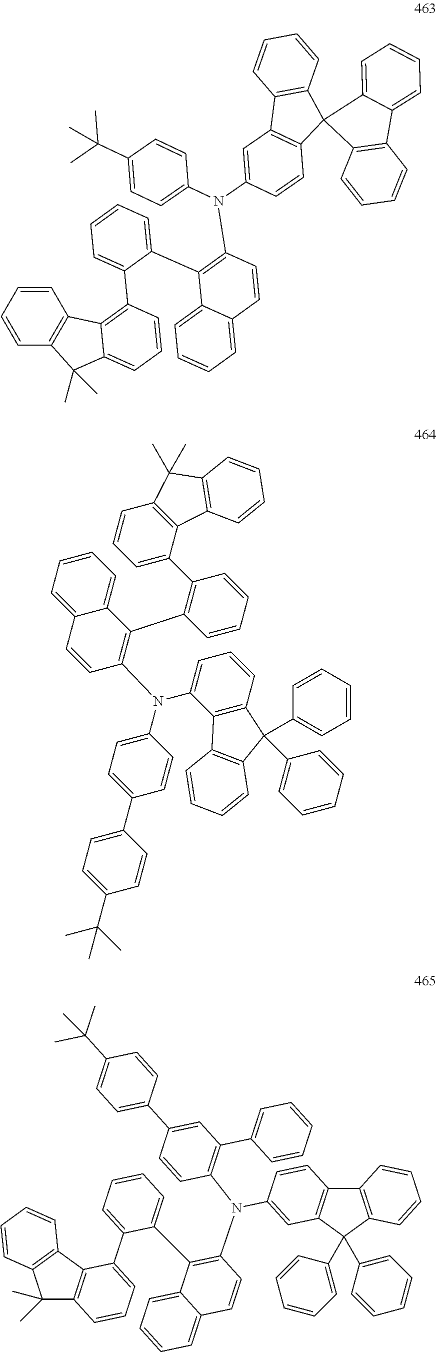 [SMILES: CC(C)(C)c1ccc(N(c2ccc3c(c2)-c2ccccc2C32c3ccccc3-c3ccccc32)c2ccc3ccccc3c2-c2ccccc2-c2cccc3c2-c2ccccc2C3(C)C)cc1 ; CC(C)(C)c1ccc(-c2ccc(N(c3cccc4c3-c3ccccc3C4(c3ccccc3)c3ccccc3)c3ccc4ccccc4c3-c3ccccc3-c3cccc4c3-c3ccccc3C4(C)C)cc2)cc1 ; CC(C)(C)c1ccc(-c2ccc(N(c3ccc4c(c3)C(c3ccccc3)(c3ccccc3)c3ccccc3-4)c3ccc4ccccc4c3-c3ccccc3-c3cccc4c3-c3ccccc3C4(C)C)c(-c3ccccc3)c2)cc1]

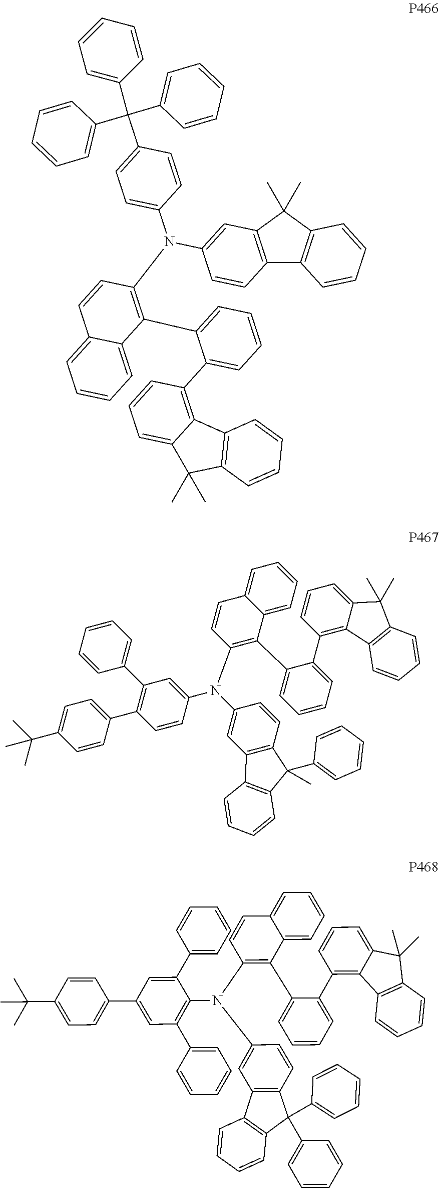 [SMILES: CC1(C)c2ccccc2-c2ccc(N(c3ccc(C(c4ccccc4)(c4ccccc4)c4ccccc4)cc3)c3ccc4ccccc4c3-c3ccccc3-c3cccc4c3-c3ccccc3C4(C)C)cc21 ; CC(C)(C)c1ccc(-c2ccc(N(c3ccc4c(c3)-c3ccccc3C4(C)c3ccccc3)c3ccc4ccccc4c3-c3ccccc3-c3cccc4c3-c3ccccc3C4(C)C)cc2-c2ccccc2)cc1 ; CC(C)(C)c1ccc(-c2cc(-c3ccccc3)c(N(c3ccc4c(c3)-c3ccccc3C4(c3ccccc3)c3ccccc3)c3ccc4ccccc4c3-c3ccccc3-c3cccc4c3-c3ccccc3C4(C)C)c(-c3ccccc3)c2)cc1]

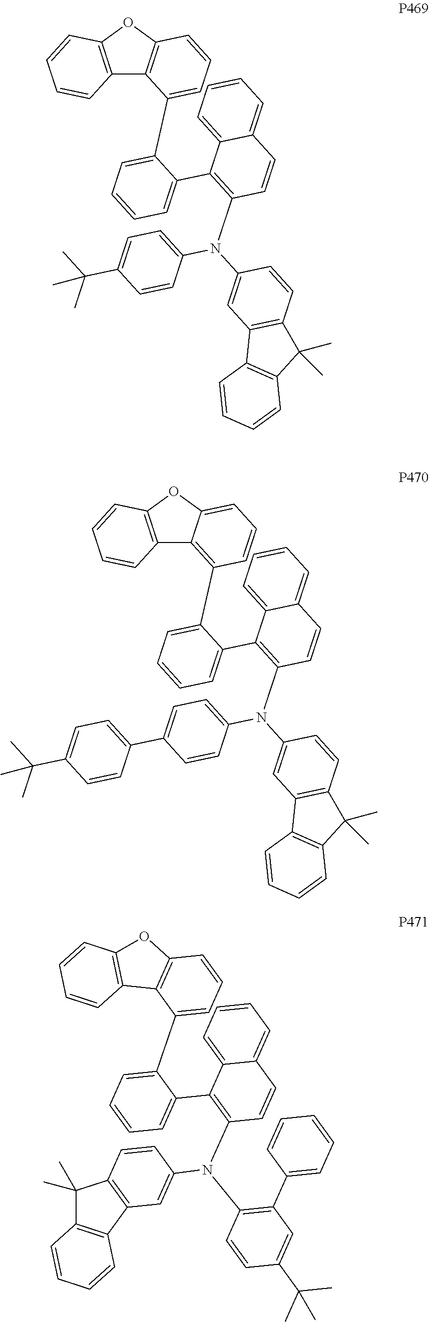

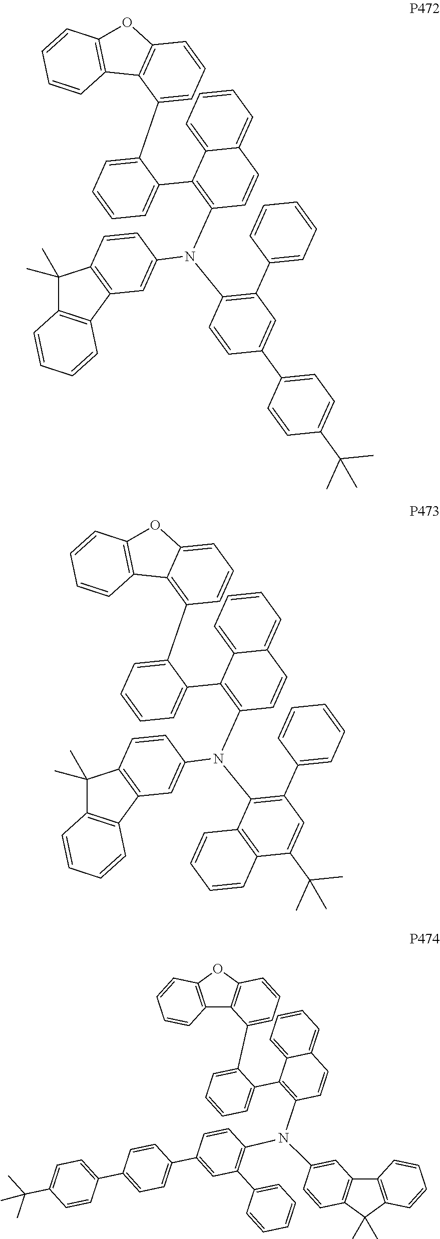

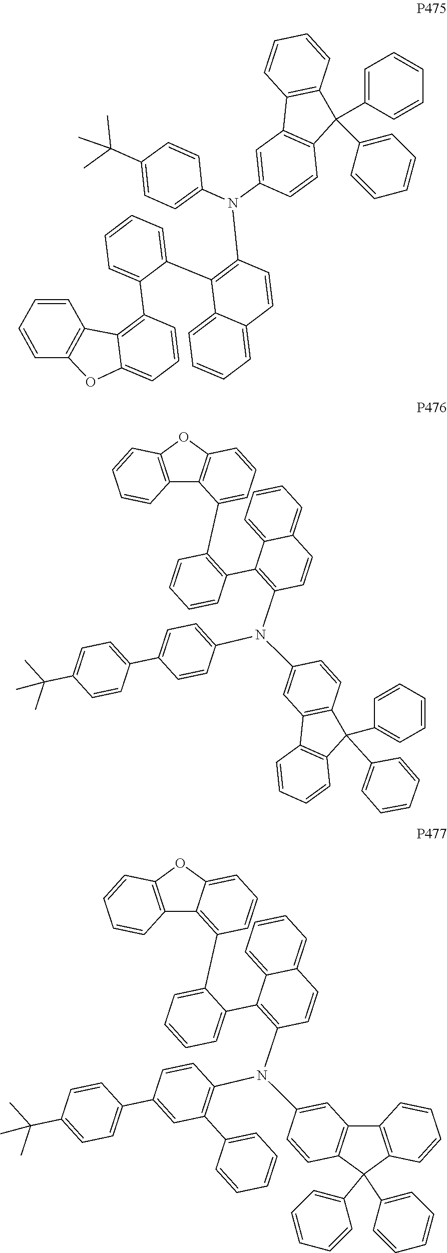 [SMILES: CC(C)(C)c1ccc(N(c2ccc3c(c2)-c2ccccc2C3(c2ccccc2)c2ccccc2)c2ccc3ccccc3c2-c2ccccc2-c2cccc3oc4ccccc4c23)cc1 ; CC(C)(C)c1ccc(-c2ccc(N(c3ccc4c(c3)-c3ccccc3C4(c3ccccc3)c3ccccc3)c3ccc4ccccc4c3-c3ccccc3-c3cccc4oc5ccccc5c34)cc2)cc1 ; CC(C)(C)c1ccc(-c2ccc(N(c3ccc4c(c3)-c3ccccc3C4(c3ccccc3)c3ccccc3)c3ccc4ccccc4c3-c3ccccc3-c3cccc4oc5ccccc5c34)c(-c3ccccc3)c2)cc1]

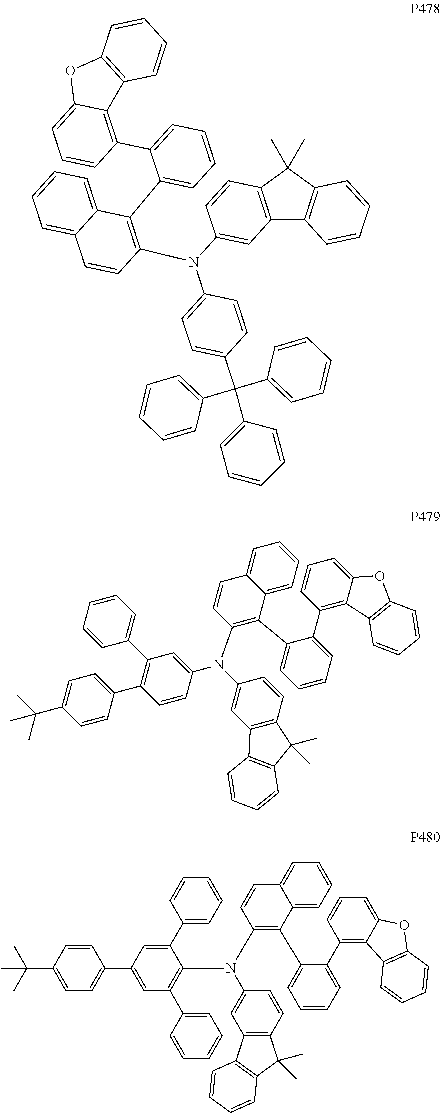 [SMILES: CC1(C)c2ccccc2-c2cc(N(c3ccc(C(c4ccccc4)(c4ccccc4)c4ccccc4)cc3)c3ccc4ccccc4c3-c3ccccc3-c3cccc4oc5ccccc5c34)ccc21 ; CC(C)(C)c1ccc(-c2ccc(N(c3ccc4c(c3)-c3ccccc3C4(C)C)c3ccc4ccccc4c3-c3ccccc3-c3cccc4oc5ccccc5c34)cc2-c2ccccc2)cc1 ; CC(C)(C)c1ccc(-c2cc(-c3ccccc3)c(N(c3ccc4c(c3)-c3ccccc3C4(C)C)c3ccc4ccccc4c3-c3ccccc3-c3cccc4oc5ccccc5c34)c(-c3ccccc3)c2)cc1]

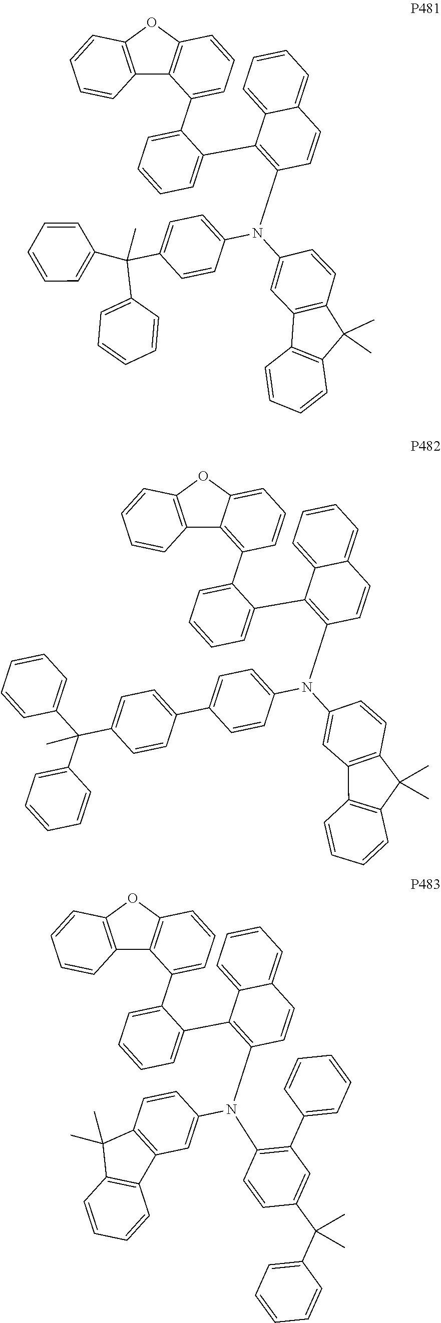 [SMILES: CC1(C)c2ccccc2-c2cc(N(c3ccc(C(C)(c4ccccc4)c4ccccc4)cc3)c3ccc4ccccc4c3-c3ccccc3-c3cccc4oc5ccccc5c34)ccc21 ; CC1(C)c2ccccc2-c2cc(N(c3ccc(-c4ccc(C(C)(c5ccccc5)c5ccccc5)cc4)cc3)c3ccc4ccccc4c3-c3ccccc3-c3cccc4oc5ccccc5c34)ccc21 ; CC(C)(c1ccccc1)c1ccc(N(c2ccc3c(c2)-c2ccccc2C3(C)C)c2ccc3ccccc3c2-c2ccccc2-c2cccc3oc4ccccc4c23)c(-c2ccccc2)c1]

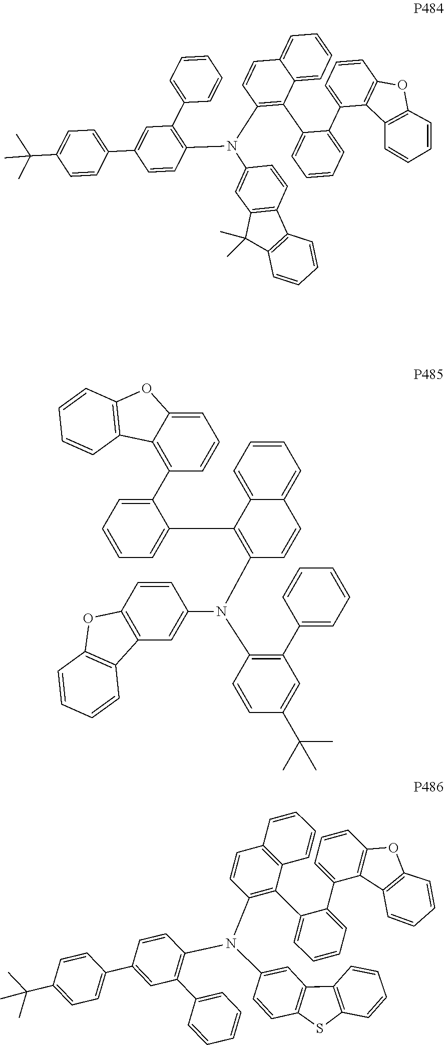 [SMILES: CC(C)(C)c1ccc(-c2ccc(N(c3ccc4c(c3)C(C)(C)c3ccccc3-4)c3ccc4ccccc4c3-c3ccccc3-c3cccc4oc5ccccc5c34)c(-c3ccccc3)c2)cc1 ; CC(C)(C)c1ccc(N(c2ccc3oc4ccccc4c3c2)c2ccc3ccccc3c2-c2ccccc2-c2cccc3oc4ccccc4c23)c(-c2ccccc2)c1 ; CC(C)(C)c1ccc(-c2ccc(N(c3ccc4sc5ccccc5c4c3)c3ccc4ccccc4c3-c3ccccc3-c3cccc4oc5ccccc5c34)c(-c3ccccc3)c2)cc1]

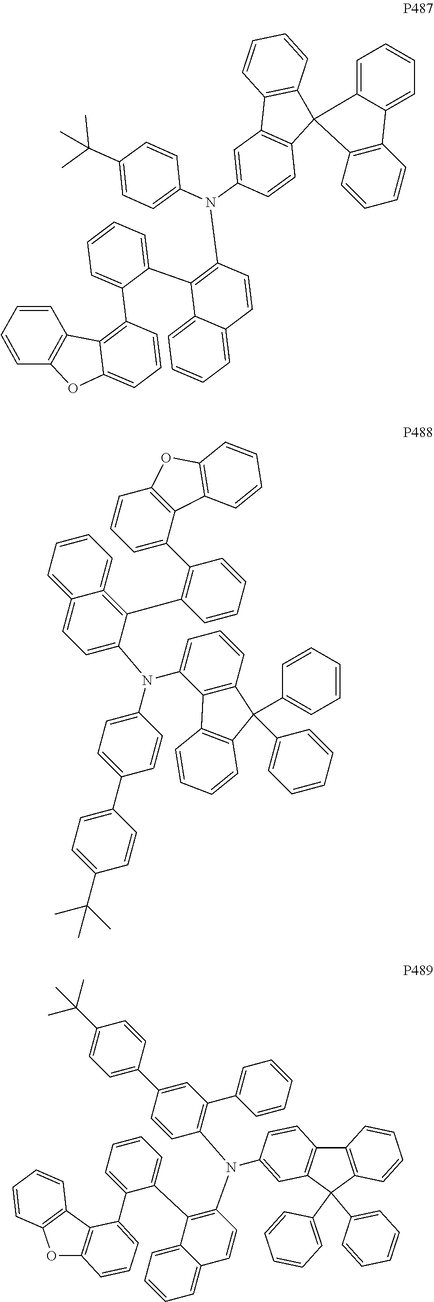

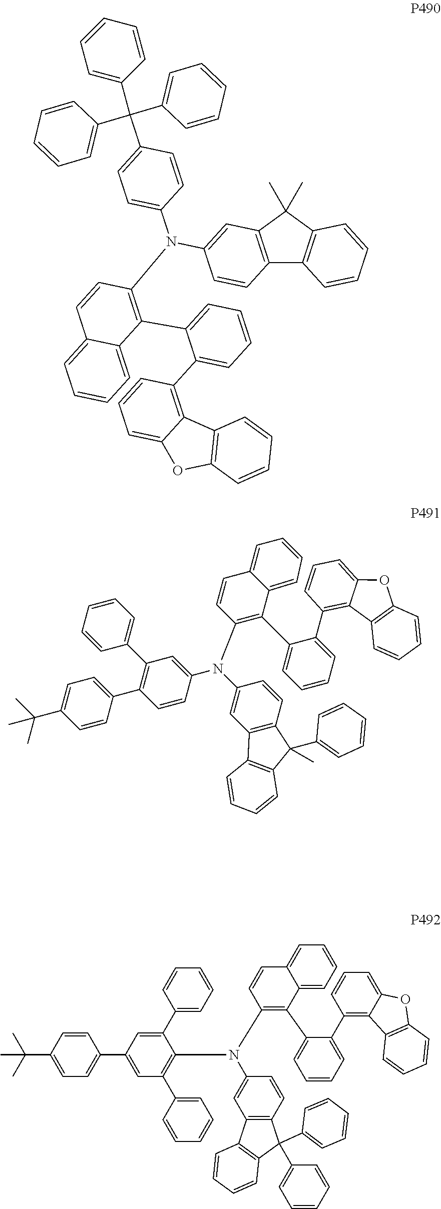 [SMILES: CC1(C)c2ccccc2-c2ccc(N(c3ccc(C(c4ccccc4)(c4ccccc4)c4ccccc4)cc3)c3ccc4ccccc4c3-c3ccccc3-c3cccc4oc5ccccc5c34)cc21 ; CC(C)(C)c1ccc(-c2ccc(N(c3ccc4c(c3)-c3ccccc3C4(C)c3ccccc3)c3ccc4ccccc4c3-c3ccccc3-c3cccc4oc5ccccc5c34)cc2-c2ccccc2)cc1 ; CC(C)(C)c1ccc(-c2cc(-c3ccccc3)c(N(c3ccc4c(c3)-c3ccccc3C4(c3ccccc3)c3ccccc3)c3ccc4ccccc4c3-c3ccccc3-c3cccc4oc5ccccc5c34)c(-c3ccccc3)c2)cc1]

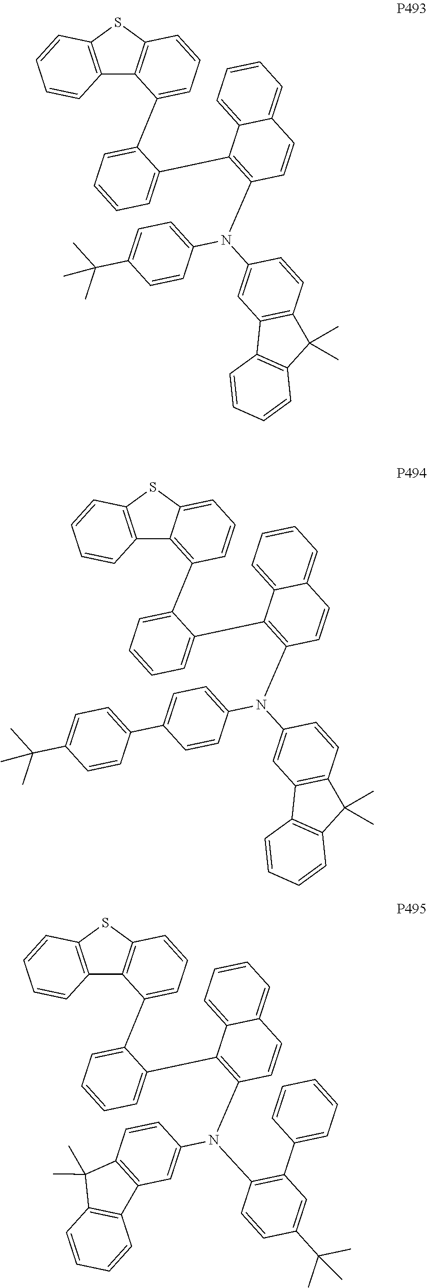

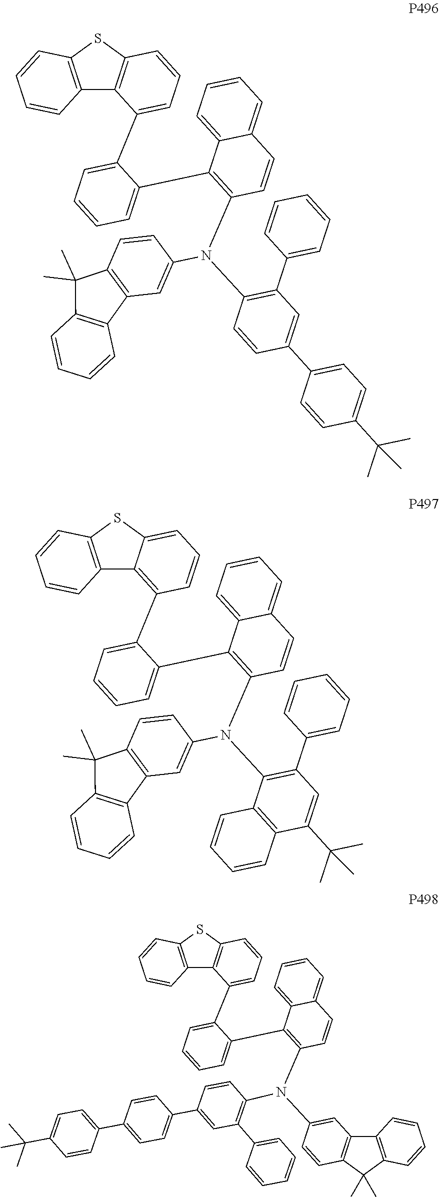

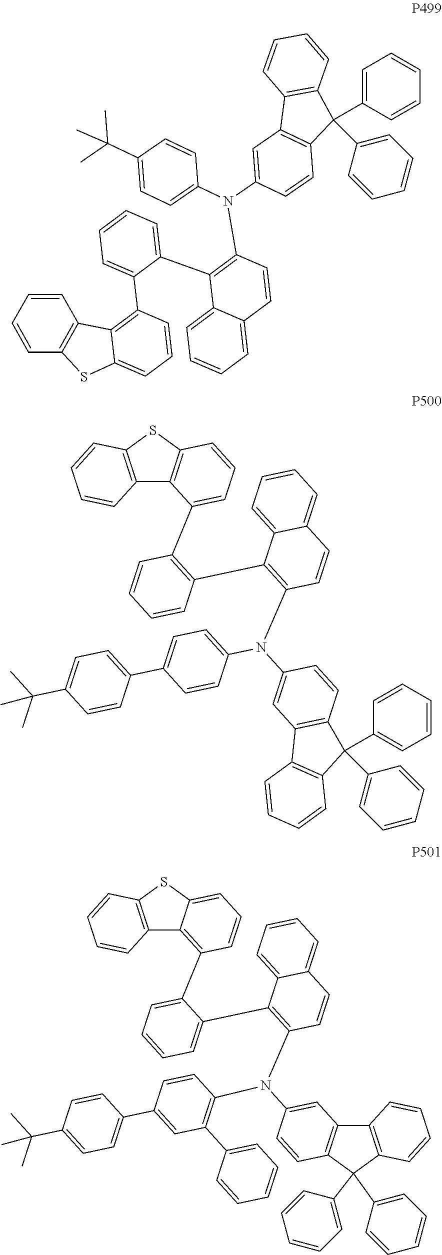 [SMILES: CC(C)(C)c1ccc(N(c2ccc3c(c2)-c2ccccc2C3(c2ccccc2)c2ccccc2)c2ccc3ccccc3c2-c2ccccc2-c2cccc3sc4ccccc4c23)cc1 ; CC(C)(C)c1ccc(-c2ccc(N(c3ccc4c(c3)-c3ccccc3C4(c3ccccc3)c3ccccc3)c3ccc4ccccc4c3-c3ccccc3-c3cccc4sc5ccccc5c34)cc2)cc1 ; CC(C)(C)c1ccc(-c2ccc(N(c3ccc4c(c3)-c3ccccc3C4(c3ccccc3)c3ccccc3)c3ccc4ccccc4c3-c3ccccc3-c3cccc4sc5ccccc5c34)c(-c3ccccc3)c2)cc1]

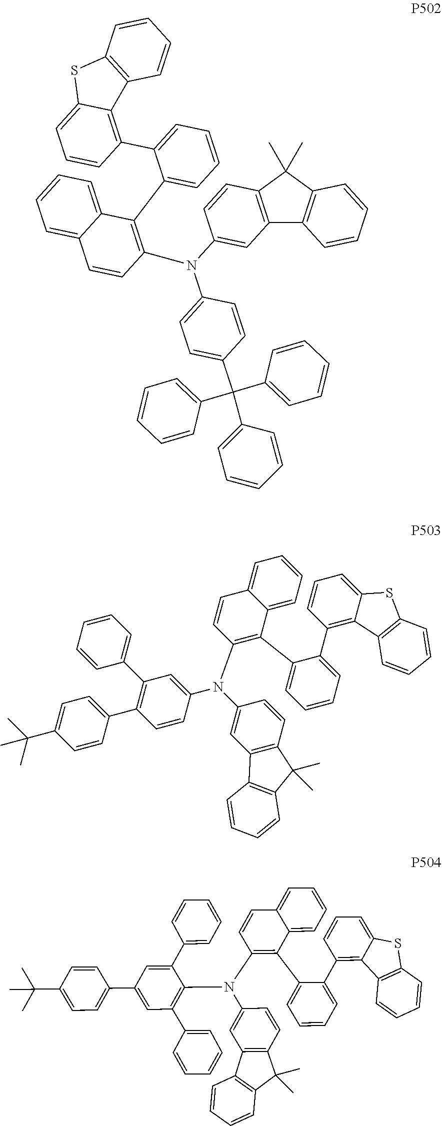 [SMILES: CC1(C)c2ccccc2-c2cc(N(c3ccc(C(c4ccccc4)(c4ccccc4)c4ccccc4)cc3)c3ccc4ccccc4c3-c3ccccc3-c3cccc4sc5ccccc5c34)ccc21 ; CC(C)(C)c1ccc(-c2ccc(N(c3ccc4c(c3)-c3ccccc3C4(C)C)c3ccc4ccccc4c3-c3ccccc3-c3cccc4sc5ccccc5c34)cc2-c2ccccc2)cc1 ; CC(C)(C)c1ccc(-c2cc(-c3ccccc3)c(N(c3ccc4c(c3)-c3ccccc3C4(C)C)c3ccc4ccccc4c3-c3ccccc3-c3cccc4sc5ccccc5c34)c(-c3ccccc3)c2)cc1]

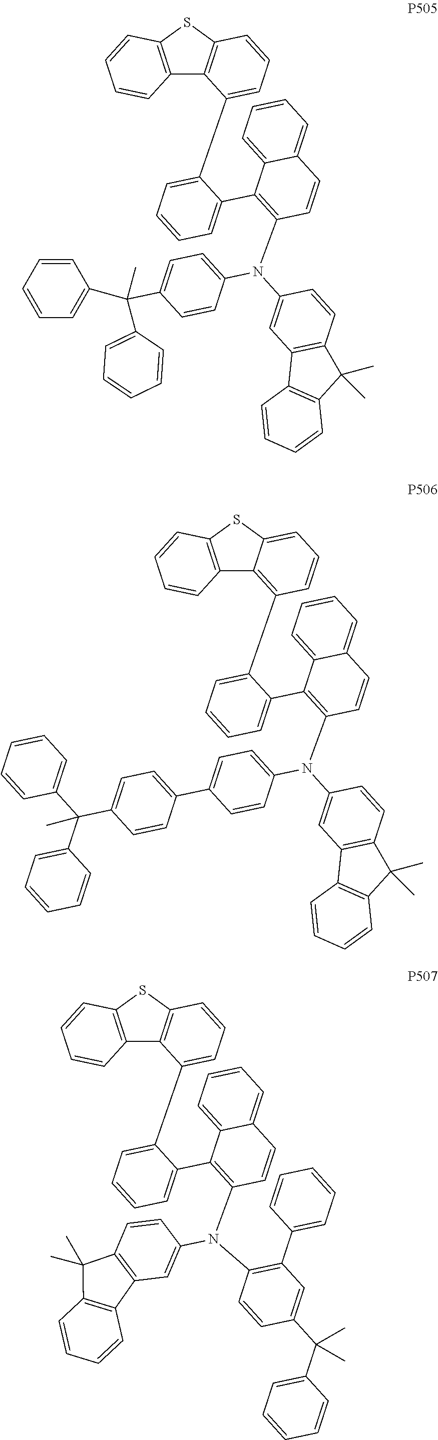

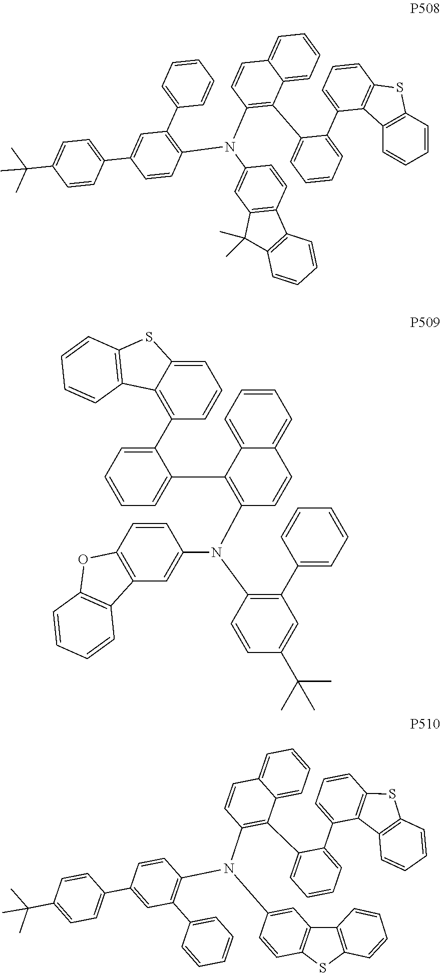 [SMILES: CC(C)(C)c1ccc(-c2ccc(N(c3ccc4c(c3)C(C)(C)c3ccccc3-4)c3ccc4ccccc4c3-c3ccccc3-c3cccc4sc5ccccc5c34)c(-c3ccccc3)c2)cc1 ; CC(C)(C)c1ccc(N(c2ccc3oc4ccccc4c3c2)c2ccc3ccccc3c2-c2ccccc2-c2cccc3sc4ccccc4c23)c(-c2ccccc2)c1 ; CC(C)(C)c1ccc(-c2ccc(N(c3ccc4sc5ccccc5c4c3)c3ccc4ccccc4c3-c3ccccc3-c3cccc4sc5ccccc5c34)c(-c3ccccc3)c2)cc1]

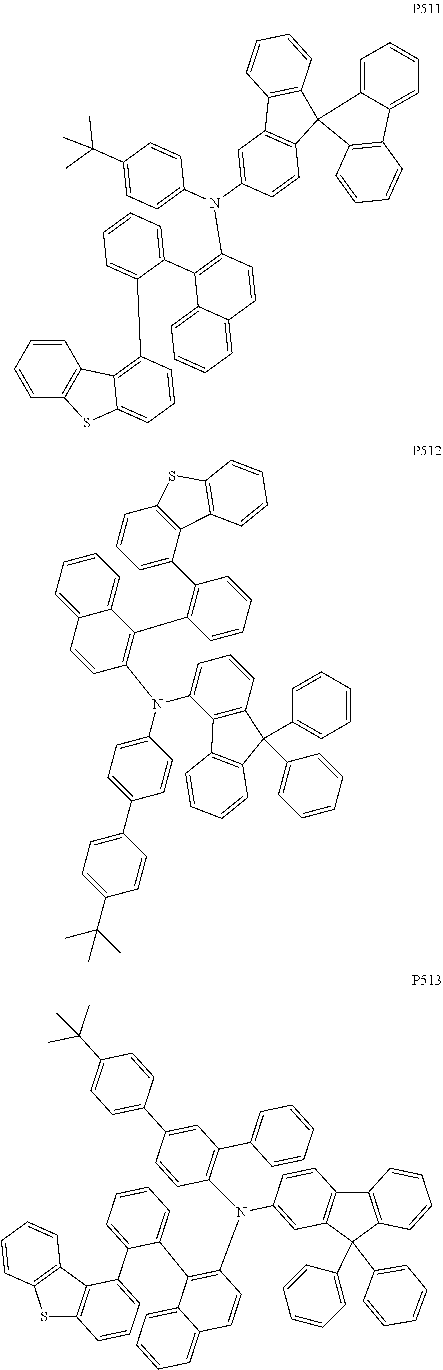

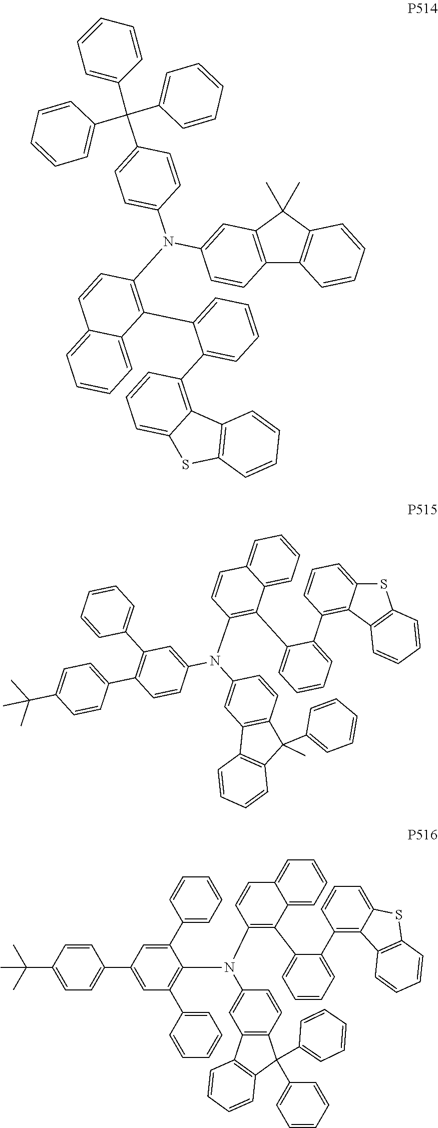 [SMILES: CC1(C)c2ccccc2-c2ccc(N(c3ccc(C(c4ccccc4)(c4ccccc4)c4ccccc4)cc3)c3ccc4ccccc4c3-c3ccccc3-c3cccc4sc5ccccc5c34)cc21 ; CC(C)(C)c1ccc(-c2ccc(N(c3ccc4c(c3)-c3ccccc3C4(C)c3ccccc3)c3ccc4ccccc4c3-c3ccccc3-c3cccc4sc5ccccc5c34)cc2-c2ccccc2)cc1 ; CC(C)(C)c1ccc(-c2cc(-c3ccccc3)c(N(c3ccc4c(c3)-c3ccccc3C4(c3ccccc3)c3ccccc3)c3ccc4ccccc4c3-c3ccccc3-c3cccc4sc5ccccc5c34)c(-c3ccccc3)c2)cc1]

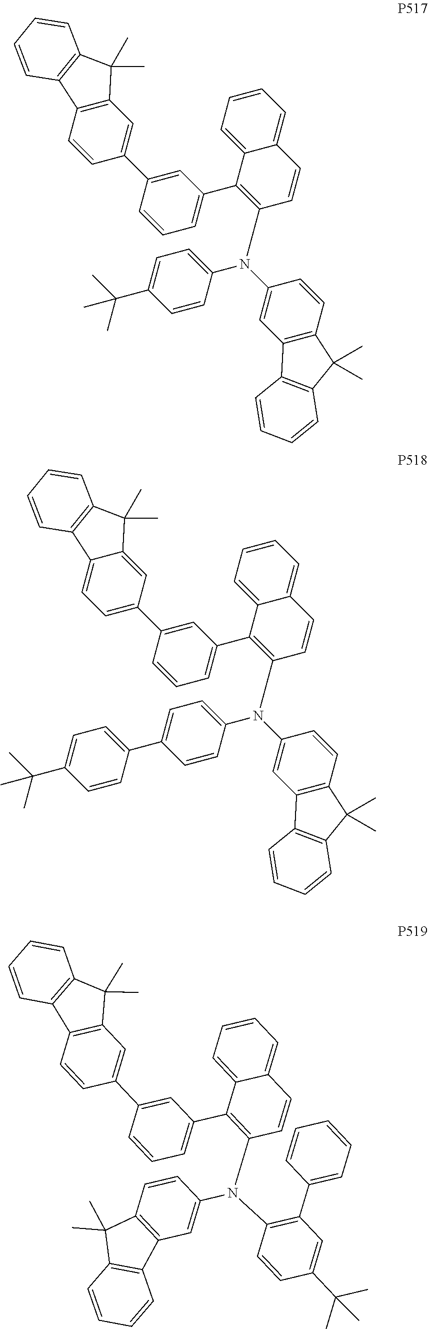

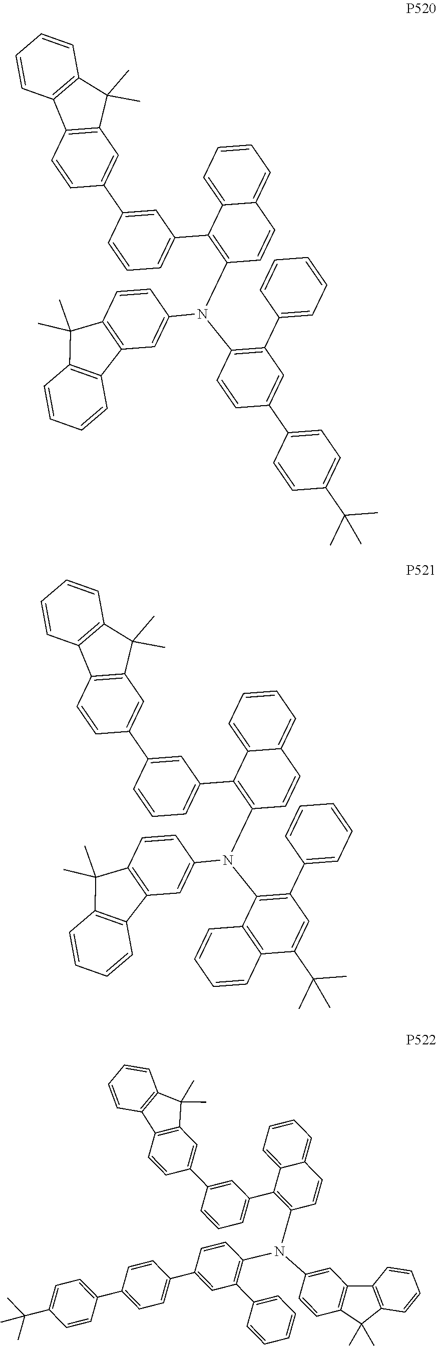

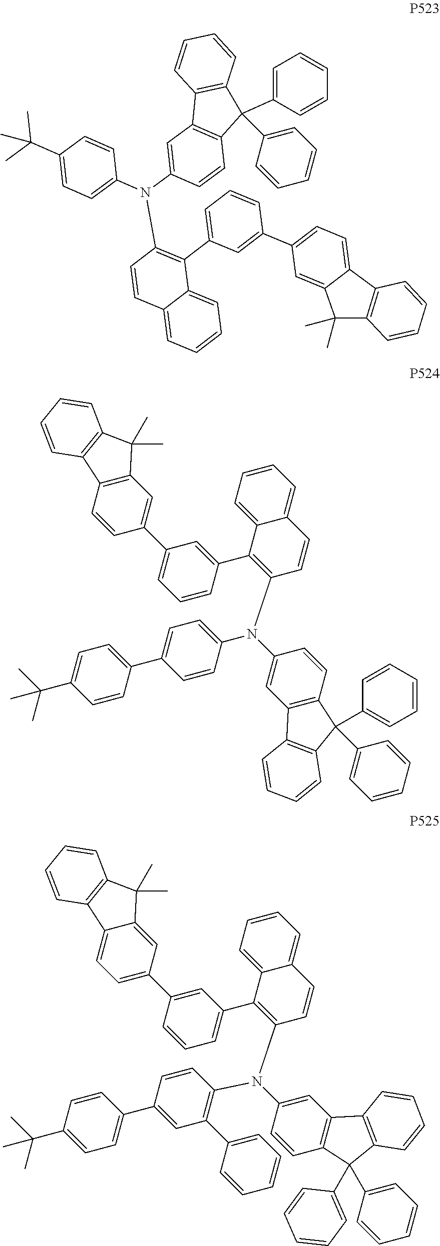 [SMILES: CC(C)(C)c1ccc(N(c2ccc3c(c2)-c2ccccc2C3(c2ccccc2)c2ccccc2)c2ccc3ccccc3c2-c2cccc(-c3ccc4c(c3)C(C)(C)c3ccccc3-4)c2)cc1 ; CC(C)(C)c1ccc(-c2ccc(N(c3ccc4c(c3)-c3ccccc3C4(c3ccccc3)c3ccccc3)c3ccc4ccccc4c3-c3cccc(-c4ccc5c(c4)C(C)(C)c4ccccc4-5)c3)cc2)cc1 ; CC(C)(C)c1ccc(-c2ccc(N(c3ccc4c(c3)-c3ccccc3C4(c3ccccc3)c3ccccc3)c3ccc4ccccc4c3-c3cccc(-c4ccc5c(c4)C(C)(C)c4ccccc4-5)c3)c(-c3ccccc3)c2)cc1]

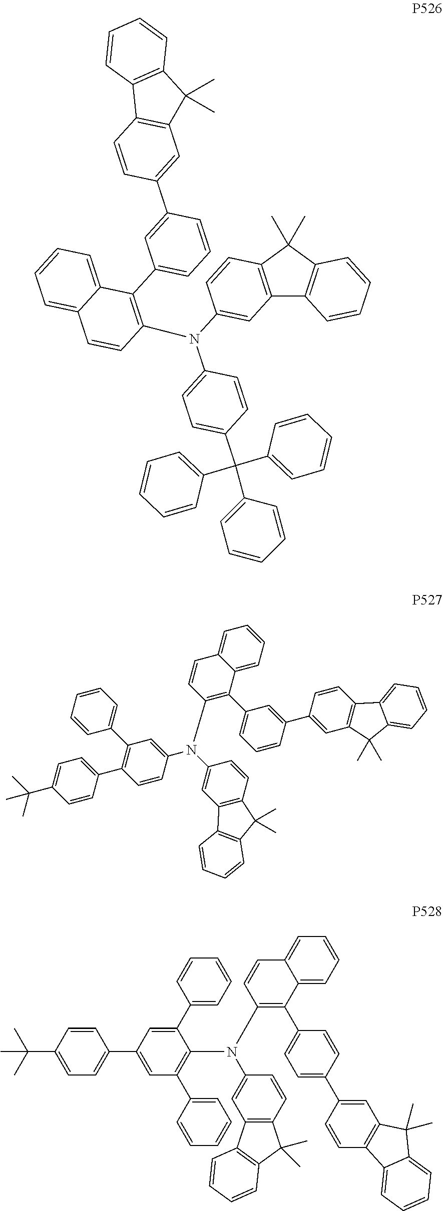 [SMILES: CC1(C)c2ccccc2-c2cc(N(c3ccc(C(c4ccccc4)(c4ccccc4)c4ccccc4)cc3)c3ccc4ccccc4c3-c3cccc(-c4ccc5c(c4)C(C)(C)c4ccccc4-5)c3)ccc21 ; CC(C)(C)c1ccc(-c2ccc(N(c3ccc4c(c3)-c3ccccc3C4(C)C)c3ccc4ccccc4c3-c3cccc(-c4ccc5c(c4)C(C)(C)c4ccccc4-5)c3)cc2-c2ccccc2)cc1 ; CC(C)(C)c1ccc(-c2cc(-c3ccccc3)c(N(c3ccc4c(c3)-c3ccccc3C4(C)C)c3ccc4ccccc4c3-c3ccc(-c4ccc5c(c4)C(C)(C)c4ccccc4-5)cc3)c(-c3ccccc3)c2)cc1]

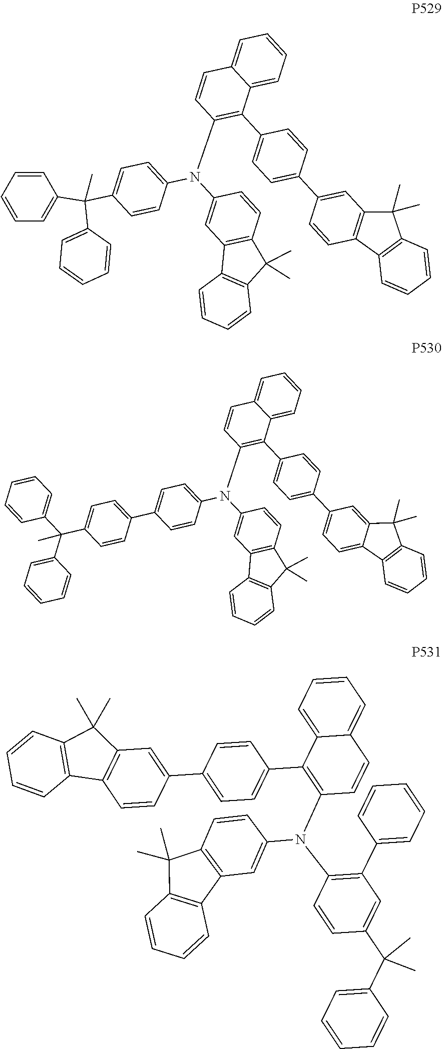 [SMILES: CC1(C)c2ccccc2-c2cc(N(c3ccc(C(C)(c4ccccc4)c4ccccc4)cc3)c3ccc4ccccc4c3-c3ccc(-c4ccc5c(c4)C(C)(C)c4ccccc4-5)cc3)ccc21 ; CC1(C)c2ccccc2-c2cc(N(c3ccc(-c4ccc(C(C)(c5ccccc5)c5ccccc5)cc4)cc3)c3ccc4ccccc4c3-c3ccc(-c4ccc5c(c4)C(C)(C)c4ccccc4-5)cc3)ccc21 ; CC(C)(c1ccccc1)c1ccc(N(c2ccc3c(c2)-c2ccccc2C3(C)C)c2ccc3ccccc3c2-c2ccc(-c3ccc4c(c3)C(C)(C)c3ccccc3-4)cc2)c(-c2ccccc2)c1]

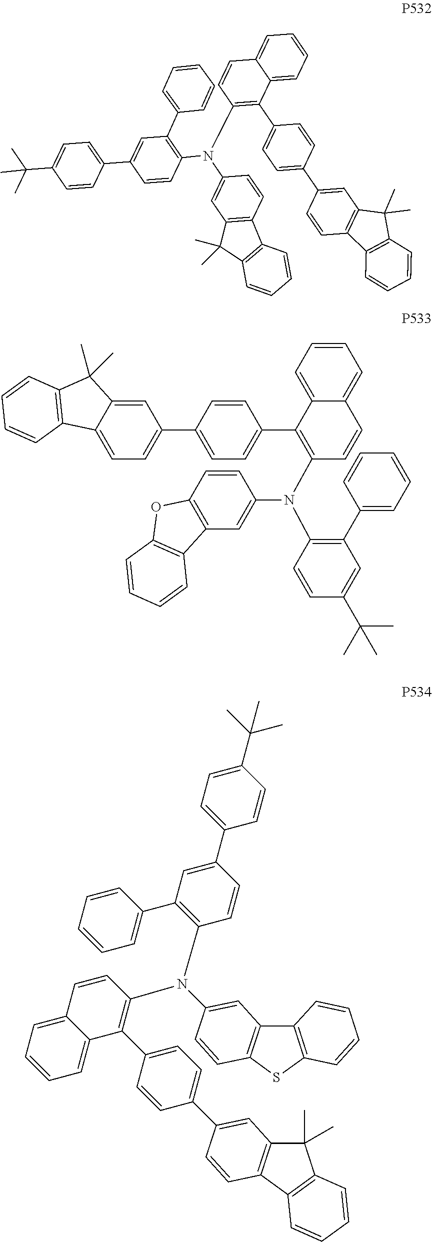 [SMILES: CC(C)(C)c1ccc(-c2ccc(N(c3ccc4c(c3)C(C)(C)c3ccccc3-4)c3ccc4ccccc4c3-c3ccc(-c4ccc5c(c4)C(C)(C)c4ccccc4-5)cc3)c(-c3ccccc3)c2)cc1 ; CC(C)(C)c1ccc(N(c2ccc3oc4ccccc4c3c2)c2ccc3ccccc3c2-c2ccc(-c3ccc4c(c3)C(C)(C)c3ccccc3-4)cc2)c(-c2ccccc2)c1 ; CC(C)(C)c1ccc(-c2ccc(N(c3ccc4sc5ccccc5c4c3)c3ccc4ccccc4c3-c3ccc(-c4ccc5c(c4)C(C)(C)c4ccccc4-5)cc3)c(-c3ccccc3)c2)cc1]

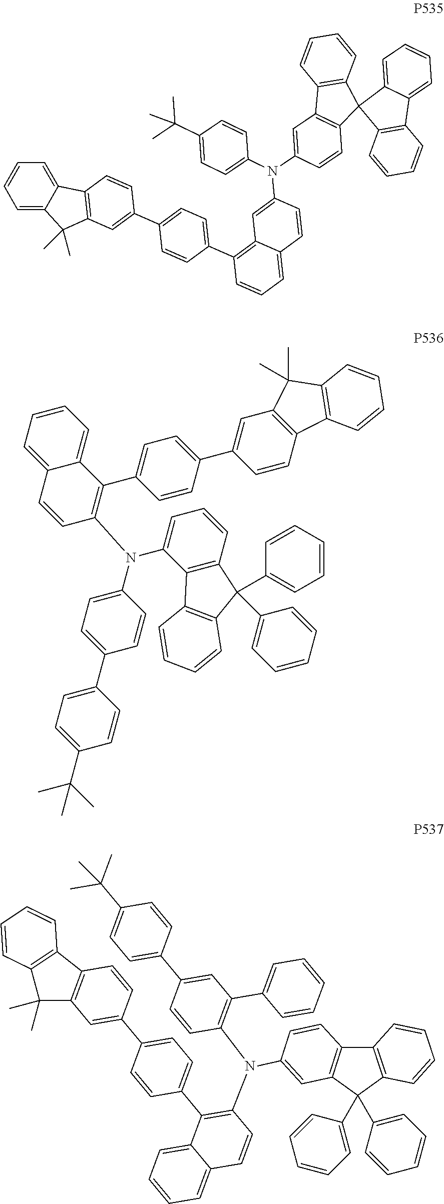 [SMILES: CC(C)(C)c1ccc(N(c2ccc3c(c2)-c2ccccc2C32c3ccccc3-c3ccccc32)c2ccc3cccc(-c4ccc(-c5ccc6c(c5)C(C)(C)c5ccccc5-6)cc4)c3c2)cc1 ; CC(C)(C)c1ccc(-c2ccc(N(c3cccc4c3-c3ccccc3C4(c3ccccc3)c3ccccc3)c3ccc4ccccc4c3-c3ccc(-c4ccc5c(c4)C(C)(C)c4ccccc4-5)cc3)cc2)cc1 ; CC(C)(C)c1ccc(-c2ccc(N(c3ccc4c(c3)C(c3ccccc3)(c3ccccc3)c3ccccc3-4)c3ccc4ccccc4c3-c3ccc(-c4ccc5c(c4)C(C)(C)c4ccccc4-5)cc3)c(-c3ccccc3)c2)cc1]

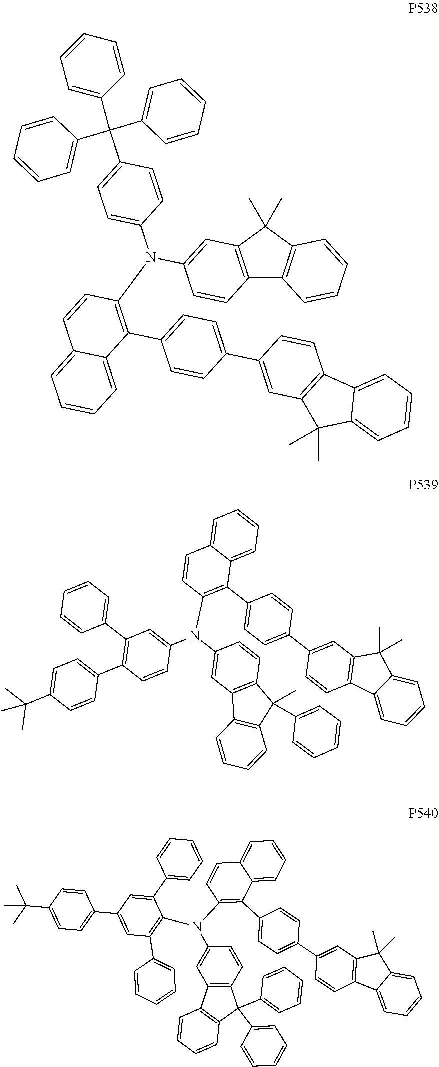 [SMILES: CC1(C)c2ccccc2-c2ccc(-c3ccc(-c4c(N(c5ccc(C(c6ccccc6)(c6ccccc6)c6ccccc6)cc5)c5ccc6c(c5)C(C)(C)c5ccccc5-6)ccc5ccccc45)cc3)cc21 ; CC(C)(C)c1ccc(-c2ccc(N(c3ccc4c(c3)-c3ccccc3C4(C)c3ccccc3)c3ccc4ccccc4c3-c3ccc(-c4ccc5c(c4)C(C)(C)c4ccccc4-5)cc3)cc2-c2ccccc2)cc1 ; CC(C)(C)c1ccc(-c2cc(-c3ccccc3)c(N(c3ccc4c(c3)-c3ccccc3C4(c3ccccc3)c3ccccc3)c3ccc4ccccc4c3-c3ccc(-c4ccc5c(c4)C(C)(C)c4ccccc4-5)cc3)c(-c3ccccc3)c2)cc1]

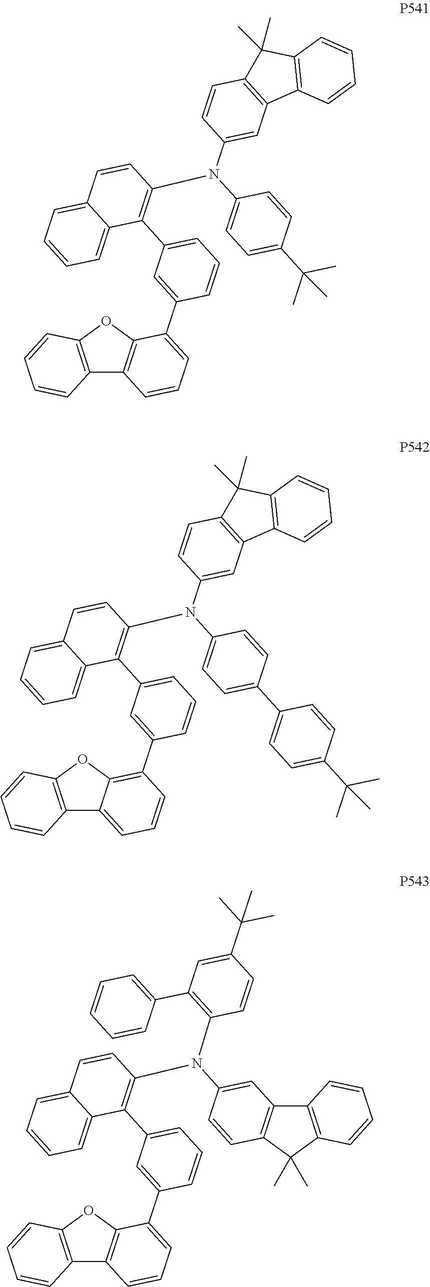 [SMILES: CC(C)(C)c1ccc(N(c2ccc3c(c2)-c2ccccc2C3(C)C)c2ccc3ccccc3c2-c2cccc(-c3cccc4c3oc3ccccc34)c2)cc1 ; CC(C)(C)c1ccc(-c2ccc(N(c3ccc4c(c3)-c3ccccc3C4(C)C)c3ccc4ccccc4c3-c3cccc(-c4cccc5c4oc4ccccc45)c3)cc2)cc1 ; CC(C)(C)c1ccc(N(c2ccc3c(c2)-c2ccccc2C3(C)C)c2ccc3ccccc3c2-c2cccc(-c3cccc4c3oc3ccccc34)c2)c(-c2ccccc2)c1]

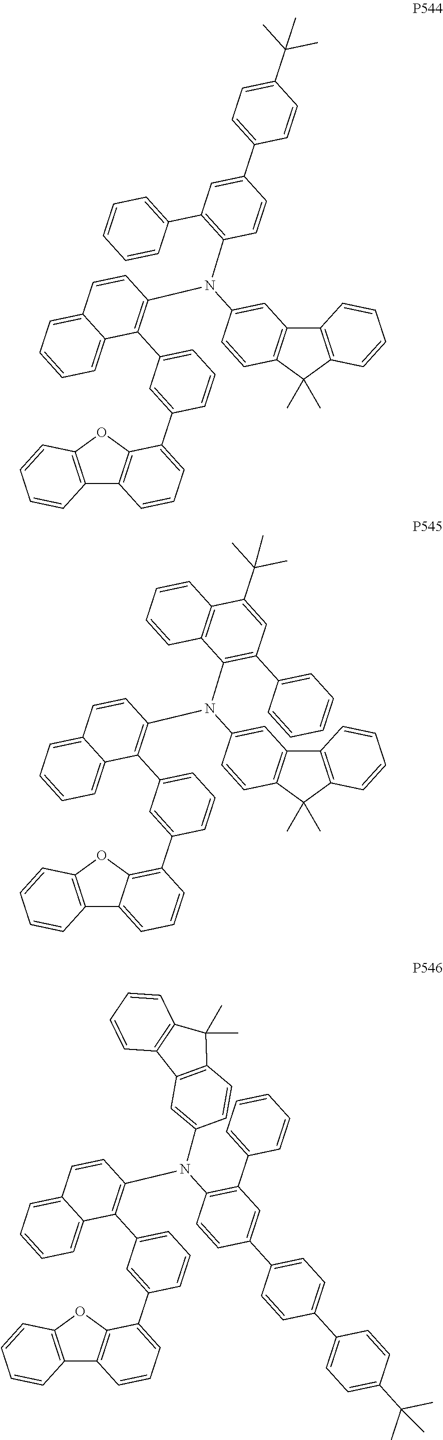 [SMILES: CC(C)(C)c1ccc(-c2ccc(N(c3ccc4c(c3)-c3ccccc3C4(C)C)c3ccc4ccccc4c3-c3cccc(-c4cccc5c4oc4ccccc45)c3)c(-c3ccccc3)c2)cc1 ; CC(C)(C)c1cc(-c2ccccc2)c(N(c2ccc3c(c2)-c2ccccc2C3(C)C)c2ccc3ccccc3c2-c2cccc(-c3cccc4c3oc3ccccc34)c2)c2ccccc12 ; CC(C)(C)c1ccc(-c2ccc(-c3ccc(N(c4ccc5c(c4)-c4ccccc4C5(C)C)c4ccc5ccccc5c4-c4cccc(-c5cccc6c5oc5ccccc56)c4)c(-c4ccccc4)c3)cc2)cc1]

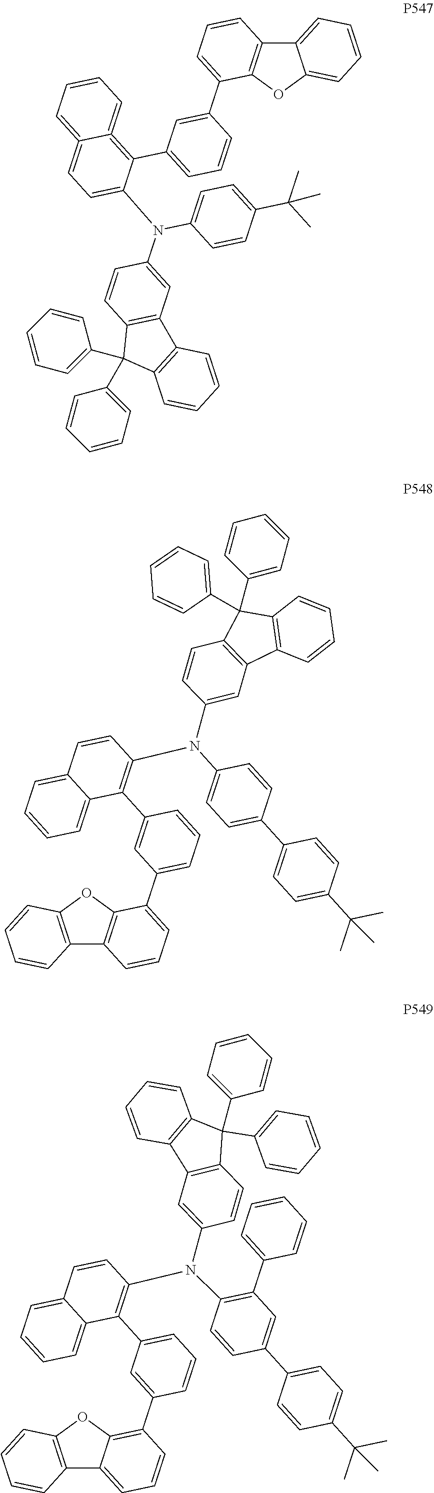

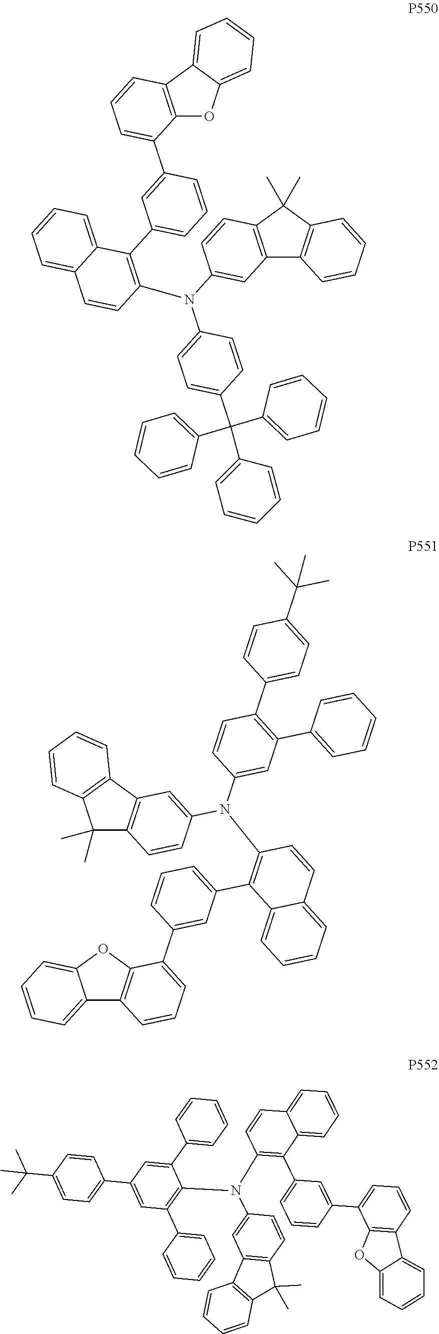

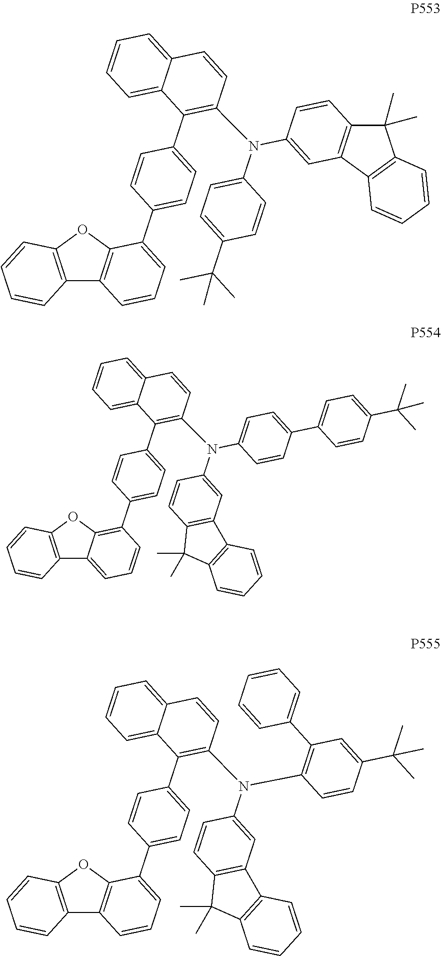 [SMILES: CC(C)(C)c1ccc(N(c2ccc3c(c2)-c2ccccc2C3(C)C)c2ccc3ccccc3c2-c2ccc(-c3cccc4c3oc3ccccc34)cc2)cc1 ; CC(C)(C)c1ccc(-c2ccc(N(c3ccc4c(c3)-c3ccccc3C4(C)C)c3ccc4ccccc4c3-c3ccc(-c4cccc5c4oc4ccccc45)cc3)cc2)cc1 ; CC(C)(C)c1ccc(N(c2ccc3c(c2)-c2ccccc2C3(C)C)c2ccc3ccccc3c2-c2ccc(-c3cccc4c3oc3ccccc34)cc2)c(-c2ccccc2)c1]

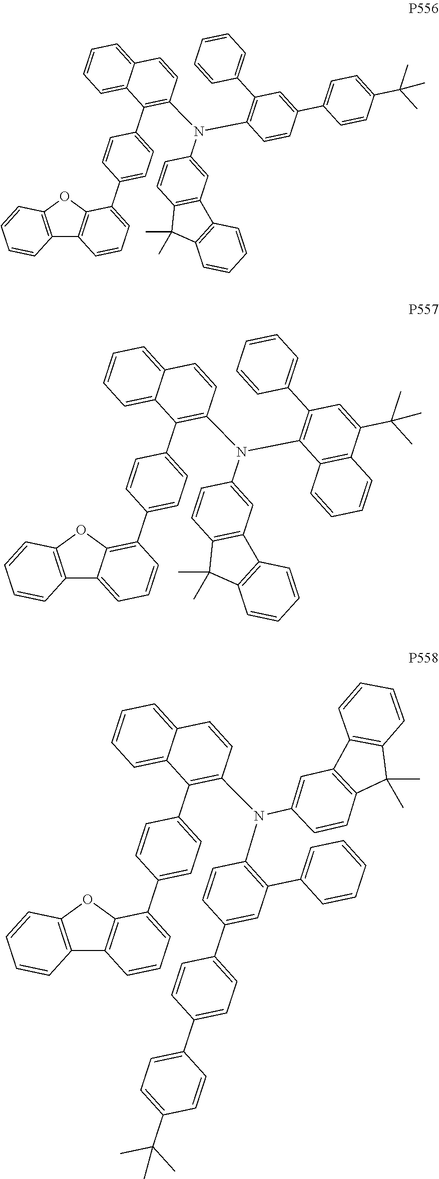 [SMILES: CC(C)(C)c1ccc(-c2ccc(N(c3ccc4c(c3)-c3ccccc3C4(C)C)c3ccc4ccccc4c3-c3ccc(-c4cccc5c4oc4ccccc45)cc3)c(-c3ccccc3)c2)cc1 ; CC(C)(C)c1cc(-c2ccccc2)c(N(c2ccc3c(c2)-c2ccccc2C3(C)C)c2ccc3ccccc3c2-c2ccc(-c3cccc4c3oc3ccccc34)cc2)c2ccccc12 ; CC(C)(C)c1ccc(-c2ccc(-c3ccc(N(c4ccc5c(c4)-c4ccccc4C5(C)C)c4ccc5ccccc5c4-c4ccc(-c5cccc6c5oc5ccccc56)cc4)c(-c4ccccc4)c3)cc2)cc1]

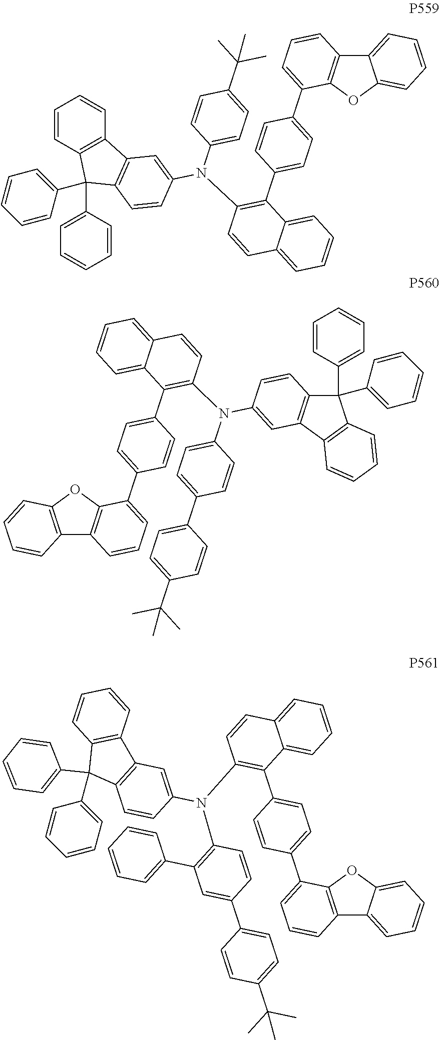 [SMILES: CC(C)(C)c1ccc(N(c2ccc3c(c2)-c2ccccc2C3(c2ccccc2)c2ccccc2)c2ccc3ccccc3c2-c2ccc(-c3cccc4c3oc3ccccc34)cc2)cc1 ; CC(C)(C)c1ccc(-c2ccc(N(c3ccc4c(c3)-c3ccccc3C4(c3ccccc3)c3ccccc3)c3ccc4ccccc4c3-c3ccc(-c4cccc5c4oc4ccccc45)cc3)cc2)cc1 ; CC(C)(C)c1ccc(-c2ccc(N(c3ccc4c(c3)-c3ccccc3C4(c3ccccc3)c3ccccc3)c3ccc4ccccc4c3-c3ccc(-c4cccc5c4oc4ccccc45)cc3)c(-c3ccccc3)c2)cc1]

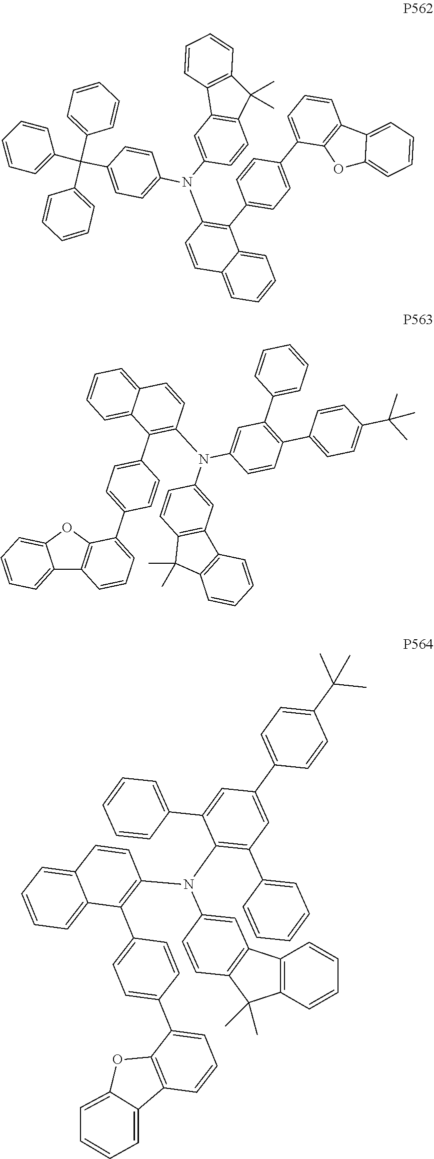 [SMILES: CC1(C)c2ccccc2-c2cc(N(c3ccc(C(c4ccccc4)(c4ccccc4)c4ccccc4)cc3)c3ccc4ccccc4c3-c3ccc(-c4cccc5c4oc4ccccc45)cc3)ccc21 ; CC(C)(C)c1ccc(-c2ccc(N(c3ccc4c(c3)-c3ccccc3C4(C)C)c3ccc4ccccc4c3-c3ccc(-c4cccc5c4oc4ccccc45)cc3)cc2-c2ccccc2)cc1 ; CC(C)(C)c1ccc(-c2cc(-c3ccccc3)c(N(c3ccc4c(c3)-c3ccccc3C4(C)C)c3ccc4ccccc4c3-c3ccc(-c4cccc5c4oc4ccccc45)cc3)c(-c3ccccc3)c2)cc1]

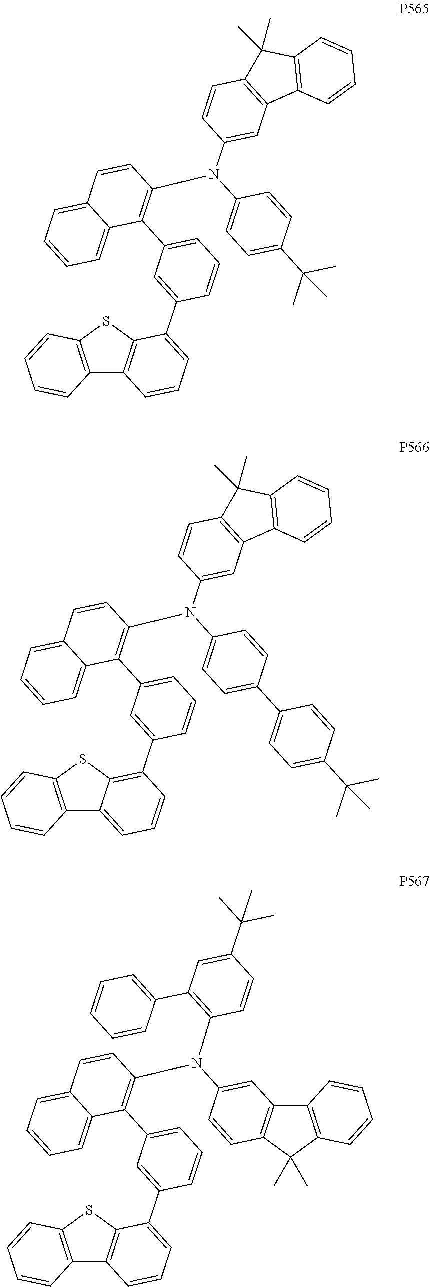 [SMILES: CC(C)(C)c1ccc(N(c2ccc3c(c2)-c2ccccc2C3(C)C)c2ccc3ccccc3c2-c2cccc(-c3cccc4c3sc3ccccc34)c2)cc1 ; CC(C)(C)c1ccc(-c2ccc(N(c3ccc4c(c3)-c3ccccc3C4(C)C)c3ccc4ccccc4c3-c3cccc(-c4cccc5c4sc4ccccc45)c3)cc2)cc1 ; CC(C)(C)c1ccc(N(c2ccc3c(c2)-c2ccccc2C3(C)C)c2ccc3ccccc3c2-c2cccc(-c3cccc4c3sc3ccccc34)c2)c(-c2ccccc2)c1]

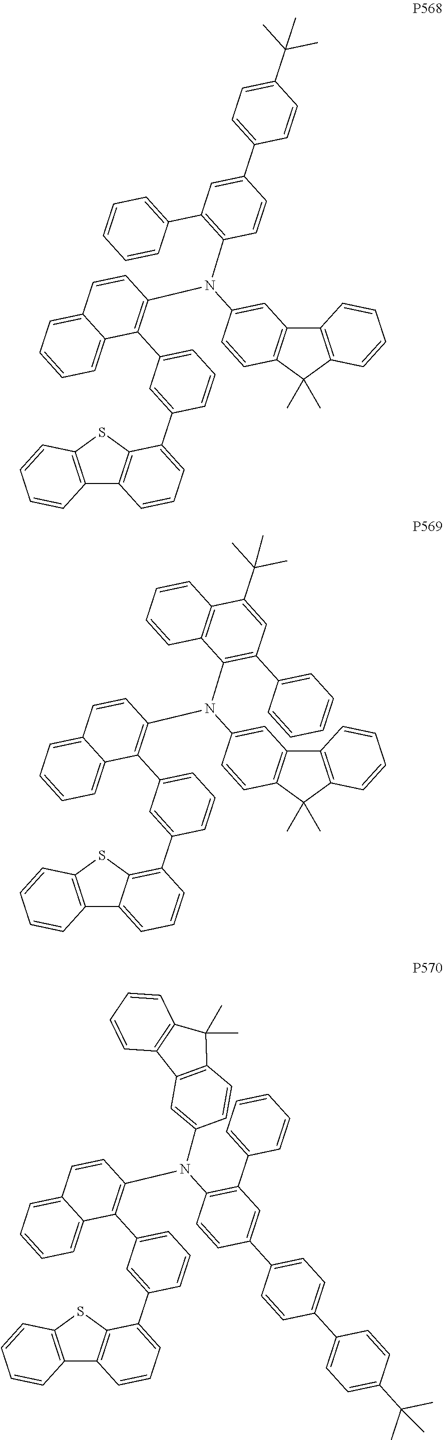 [SMILES: CC(C)(C)c1ccc(-c2ccc(N(c3ccc4c(c3)-c3ccccc3C4(C)C)c3ccc4ccccc4c3-c3cccc(-c4cccc5c4sc4ccccc45)c3)c(-c3ccccc3)c2)cc1 ; CC(C)(C)c1cc(-c2ccccc2)c(N(c2ccc3c(c2)-c2ccccc2C3(C)C)c2ccc3ccccc3c2-c2cccc(-c3cccc4c3sc3ccccc34)c2)c2ccccc12 ; CC(C)(C)c1ccc(-c2ccc(-c3ccc(N(c4ccc5c(c4)-c4ccccc4C5(C)C)c4ccc5ccccc5c4-c4cccc(-c5cccc6c5sc5ccccc56)c4)c(-c4ccccc4)c3)cc2)cc1]

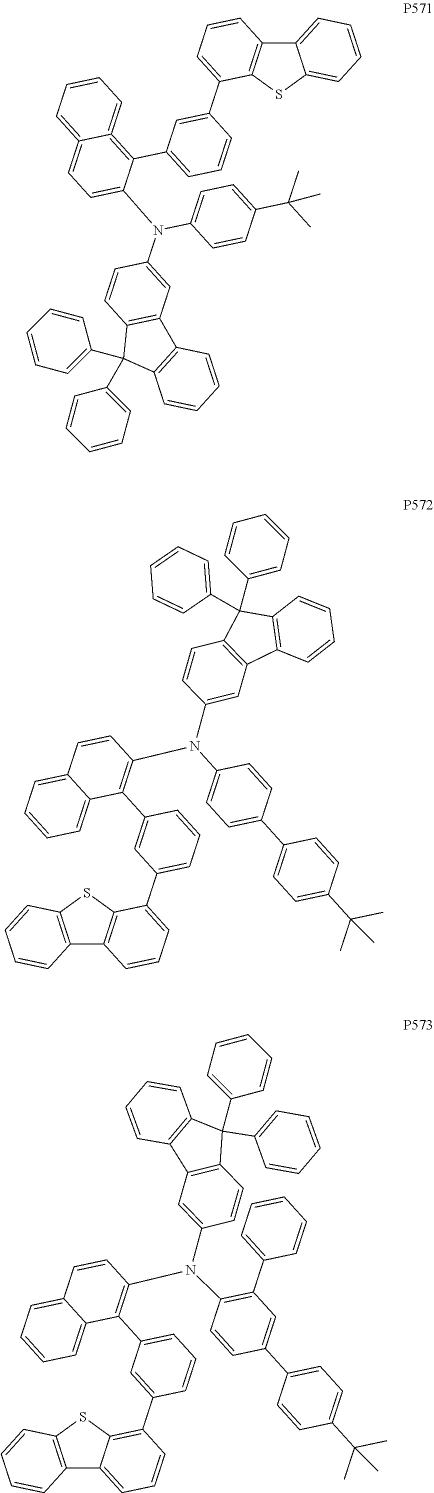

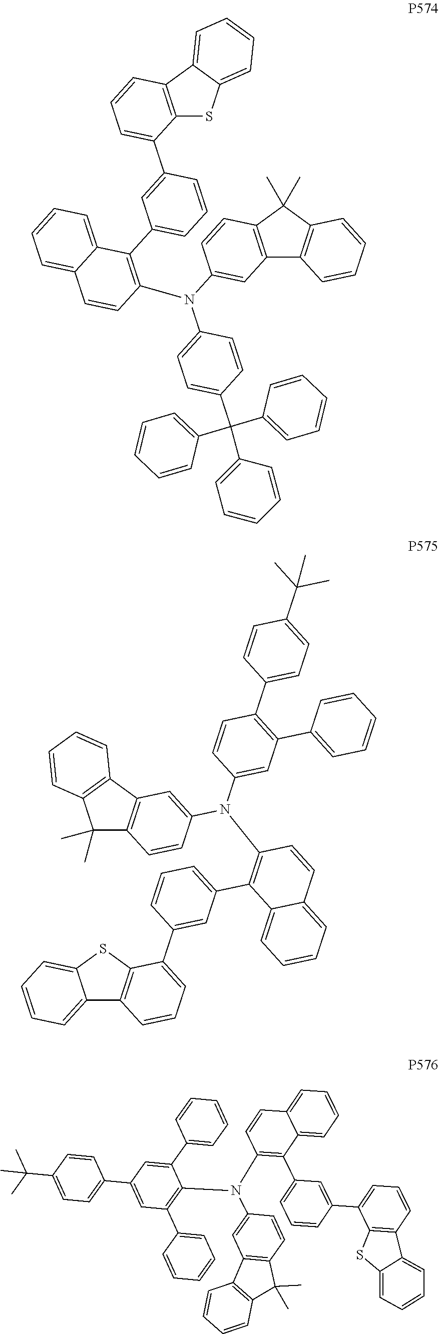

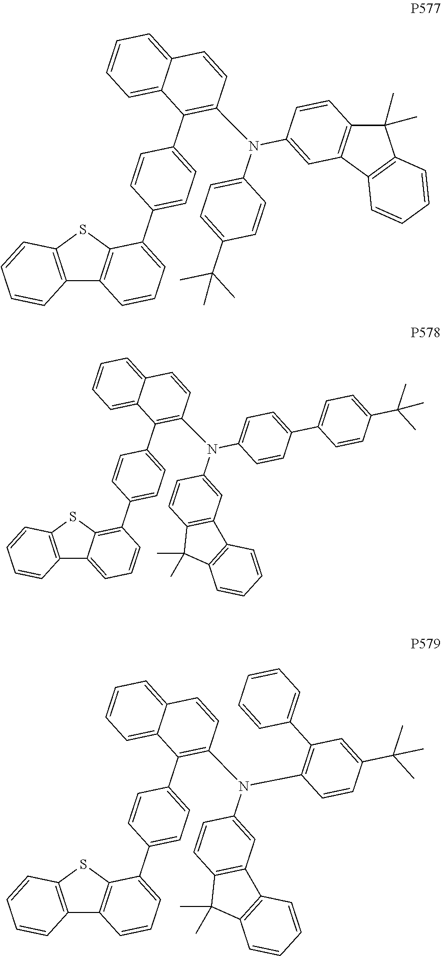 [SMILES: CC(C)(C)c1ccc(N(c2ccc3c(c2)-c2ccccc2C3(C)C)c2ccc3ccccc3c2-c2ccc(-c3cccc4c3sc3ccccc34)cc2)cc1 ; CC(C)(C)c1ccc(-c2ccc(N(c3ccc4c(c3)-c3ccccc3C4(C)C)c3ccc4ccccc4c3-c3ccc(-c4cccc5c4sc4ccccc45)cc3)cc2)cc1 ; CC(C)(C)c1ccc(N(c2ccc3c(c2)-c2ccccc2C3(C)C)c2ccc3ccccc3c2-c2ccc(-c3cccc4c3sc3ccccc34)cc2)c(-c2ccccc2)c1]

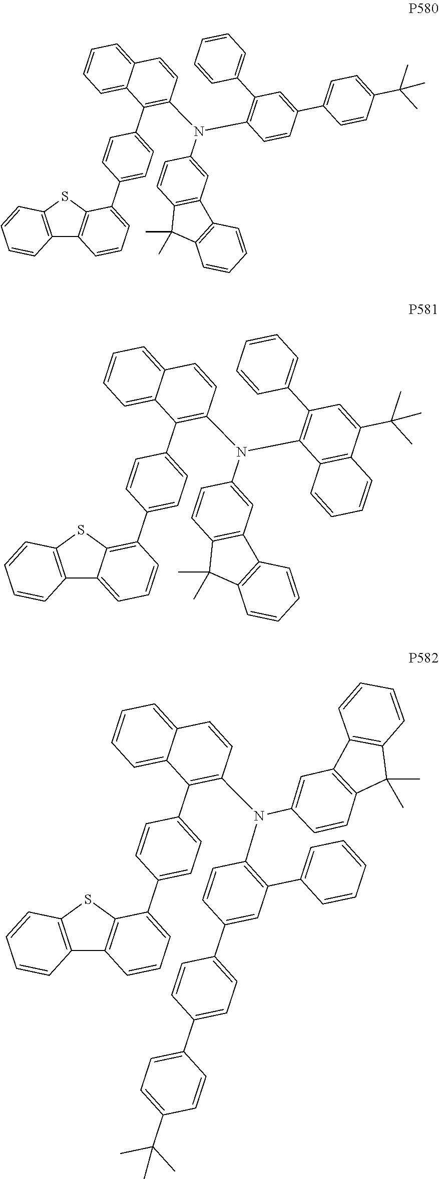 [SMILES: CC(C)(C)c1ccc(-c2ccc(N(c3ccc4c(c3)-c3ccccc3C4(C)C)c3ccc4ccccc4c3-c3ccc(-c4cccc5c4sc4ccccc45)cc3)c(-c3ccccc3)c2)cc1 ; CC(C)(C)c1cc(-c2ccccc2)c(N(c2ccc3c(c2)-c2ccccc2C3(C)C)c2ccc3ccccc3c2-c2ccc(-c3cccc4c3sc3ccccc34)cc2)c2ccccc12 ; CC(C)(C)c1ccc(-c2ccc(-c3ccc(N(c4ccc5c(c4)-c4ccccc4C5(C)C)c4ccc5ccccc5c4-c4ccc(-c5cccc6c5sc5ccccc56)cc4)c(-c4ccccc4)c3)cc2)cc1]

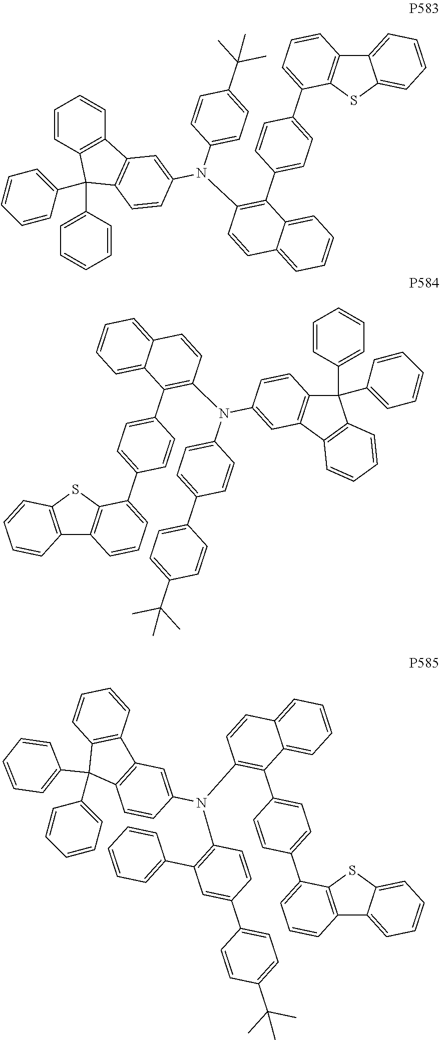 [SMILES: CC(C)(C)c1ccc(N(c2ccc3c(c2)-c2ccccc2C3(c2ccccc2)c2ccccc2)c2ccc3ccccc3c2-c2ccc(-c3cccc4c3sc3ccccc34)cc2)cc1 ; CC(C)(C)c1ccc(-c2ccc(N(c3ccc4c(c3)-c3ccccc3C4(c3ccccc3)c3ccccc3)c3ccc4ccccc4c3-c3ccc(-c4cccc5c4sc4ccccc45)cc3)cc2)cc1 ; CC(C)(C)c1ccc(-c2ccc(N(c3ccc4c(c3)-c3ccccc3C4(c3ccccc3)c3ccccc3)c3ccc4ccccc4c3-c3ccc(-c4cccc5c4sc4ccccc45)cc3)c(-c3ccccc3)c2)cc1]

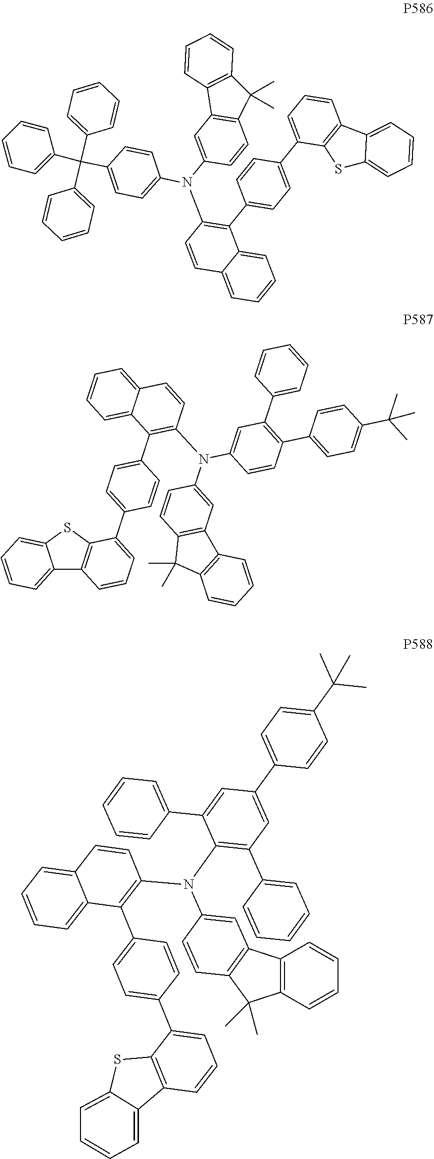 [SMILES: CC1(C)c2ccccc2-c2cc(N(c3ccc(C(c4ccccc4)(c4ccccc4)c4ccccc4)cc3)c3ccc4ccccc4c3-c3ccc(-c4cccc5c4sc4ccccc45)cc3)ccc21 ; CC(C)(C)c1ccc(-c2ccc(N(c3ccc4c(c3)-c3ccccc3C4(C)C)c3ccc4ccccc4c3-c3ccc(-c4cccc5c4sc4ccccc45)cc3)cc2-c2ccccc2)cc1 ; CC(C)(C)c1ccc(-c2cc(-c3ccccc3)c(N(c3ccc4c(c3)-c3ccccc3C4(C)C)c3ccc4ccccc4c3-c3ccc(-c4cccc5c4sc4ccccc45)cc3)c(-c3ccccc3)c2)cc1]

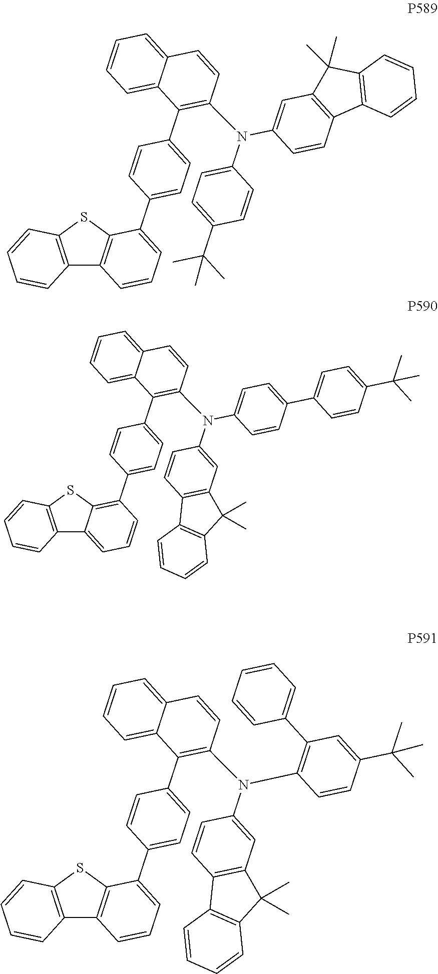

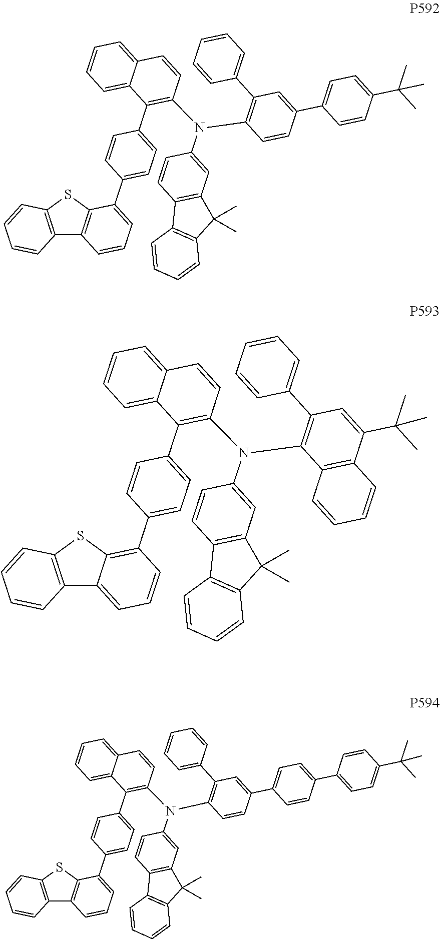 [SMILES: CC(C)(C)c1ccc(-c2ccc(N(c3ccc4c(c3)C(C)(C)c3ccccc3-4)c3ccc4ccccc4c3-c3ccc(-c4cccc5c4sc4ccccc45)cc3)c(-c3ccccc3)c2)cc1 ; CC(C)(C)c1cc(-c2ccccc2)c(N(c2ccc3c(c2)C(C)(C)c2ccccc2-3)c2ccc3ccccc3c2-c2ccc(-c3cccc4c3sc3ccccc34)cc2)c2ccccc12 ; CC(C)(C)c1ccc(-c2ccc(-c3ccc(N(c4ccc5c(c4)C(C)(C)c4ccccc4-5)c4ccc5ccccc5c4-c4ccc(-c5cccc6c5sc5ccccc56)cc4)c(-c4ccccc4)c3)cc2)cc1]

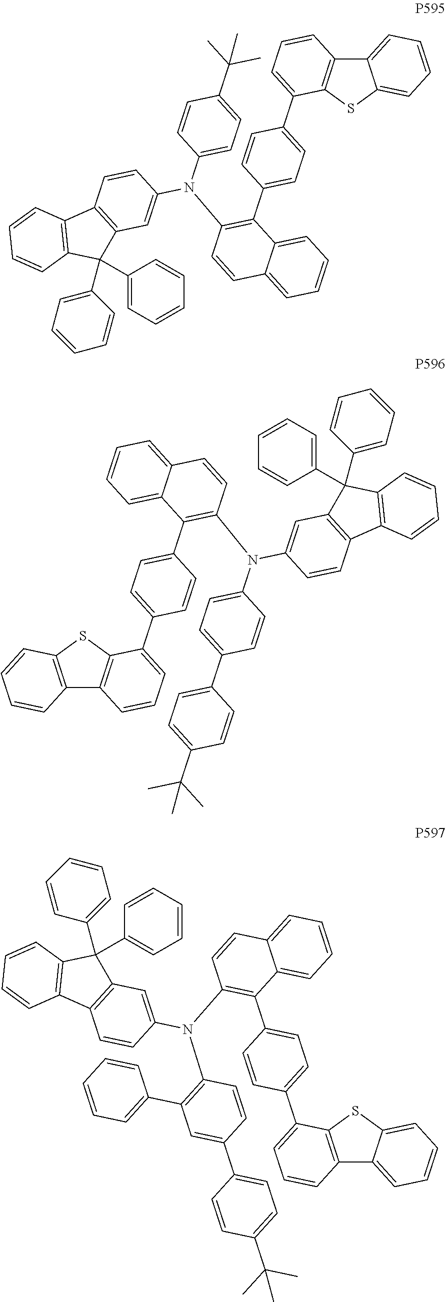 [SMILES: CC(C)(C)c1ccc(N(c2ccc3c(c2)C(c2ccccc2)(c2ccccc2)c2ccccc2-3)c2ccc3ccccc3c2-c2ccc(-c3cccc4c3sc3ccccc34)cc2)cc1 ; CC(C)(C)c1ccc(-c2ccc(N(c3ccc4c(c3)C(c3ccccc3)(c3ccccc3)c3ccccc3-4)c3ccc4ccccc4c3-c3ccc(-c4cccc5c4sc4ccccc45)cc3)cc2)cc1 ; CC(C)(C)c1ccc(-c2ccc(N(c3ccc4c(c3)C(c3ccccc3)(c3ccccc3)c3ccccc3-4)c3ccc4ccccc4c3-c3ccc(-c4cccc5c4sc4ccccc45)cc3)c(-c3ccccc3)c2)cc1]

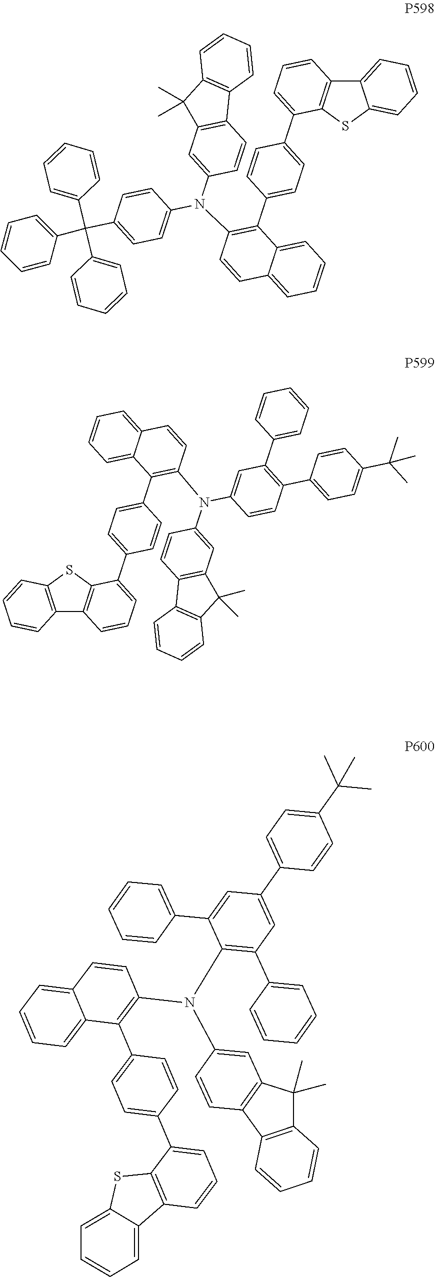 [SMILES: CC1(C)c2ccccc2-c2ccc(N(c3ccc(C(c4ccccc4)(c4ccccc4)c4ccccc4)cc3)c3ccc4ccccc4c3-c3ccc(-c4cccc5c4sc4ccccc45)cc3)cc21 ; CC(C)(C)c1ccc(-c2ccc(N(c3ccc4c(c3)C(C)(C)c3ccccc3-4)c3ccc4ccccc4c3-c3ccc(-c4cccc5c4sc4ccccc45)cc3)cc2-c2ccccc2)cc1 ; CC(C)(C)c1ccc(-c2cc(-c3ccccc3)c(N(c3ccc4c(c3)C(C)(C)c3ccccc3-4)c3ccc4ccccc4c3-c3ccc(-c4cccc5c4sc4ccccc45)cc3)c(-c3ccccc3)c2)cc1]

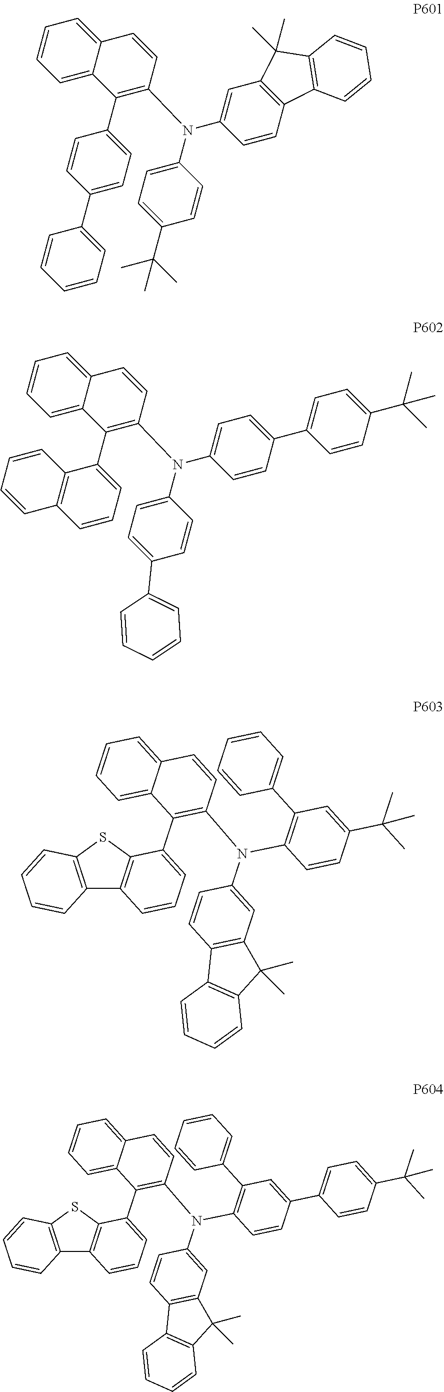 [SMILES: CC(C)(C)c1ccc(N(c2ccc3c(c2)C(C)(C)c2ccccc2-3)c2ccc3ccccc3c2-c2ccc(-c3ccccc3)cc2)cc1 ; CC(C)(C)c1ccc(-c2ccc(N(c3ccc(-c4ccccc4)cc3)c3ccc4ccccc4c3-c3cccc4ccccc34)cc2)cc1 ; CC(C)(C)c1ccc(N(c2ccc3c(c2)C(C)(C)c2ccccc2-3)c2ccc3ccccc3c2-c2cccc3c2sc2ccccc23)c(-c2ccccc2)c1 ; CC(C)(C)c1ccc(-c2ccc(N(c3ccc4c(c3)C(C)(C)c3ccccc3-4)c3ccc4ccccc4c3-c3cccc4c3sc3ccccc34)c(-c3ccccc3)c2)cc1]

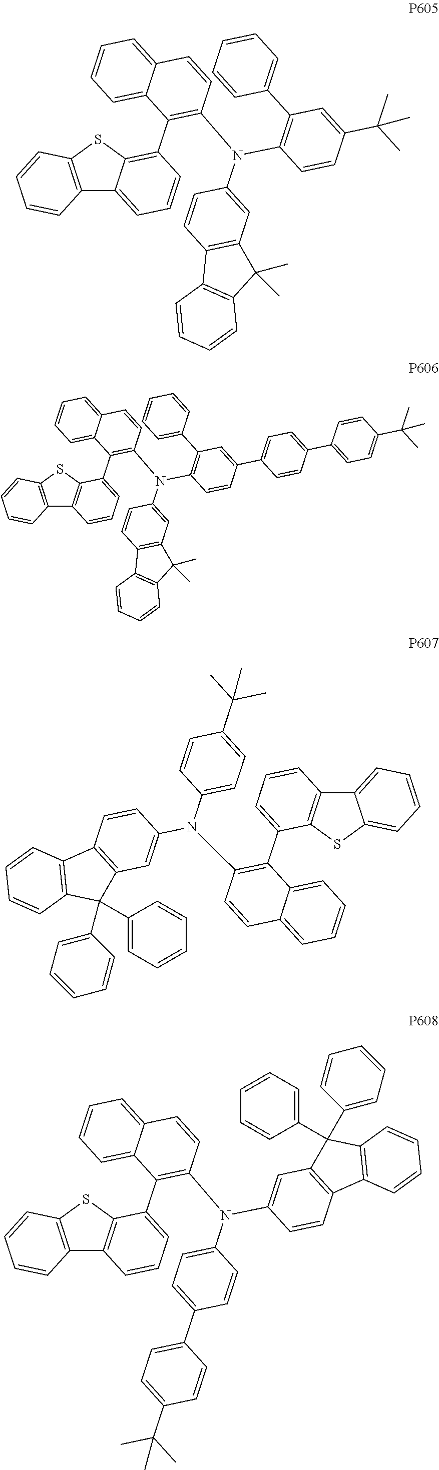 [SMILES: CC(C)(C)c1ccc(N(c2ccc3c(c2)C(C)(C)c2ccccc2-3)c2ccc3ccccc3c2-c2cccc3c2sc2ccccc23)c(-c2ccccc2)c1 ; CC(C)(C)c1ccc(-c2ccc(-c3ccc(N(c4ccc5c(c4)C(C)(C)c4ccccc4-5)c4ccc5ccccc5c4-c4cccc5c4sc4ccccc45)c(-c4ccccc4)c3)cc2)cc1 ; CC(C)(C)c1ccc(N(c2ccc3c(c2)C(c2ccccc2)(c2ccccc2)c2ccccc2-3)c2ccc3ccccc3c2-c2cccc3c2sc2ccccc23)cc1 ; CC(C)(C)c1ccc(-c2ccc(N(c3ccc4c(c3)C(c3ccccc3)(c3ccccc3)c3ccccc3-4)c3ccc4ccccc4c3-c3cccc4c3sc3ccccc34)cc2)cc1]

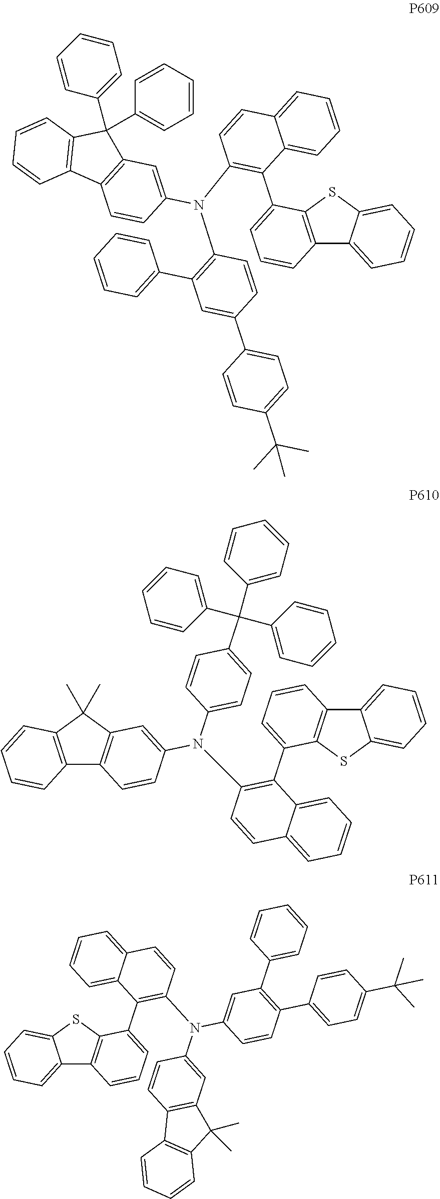 [SMILES: CC(C)(C)c1ccc(-c2ccc(N(c3ccc4c(c3)C(c3ccccc3)(c3ccccc3)c3ccccc3-4)c3ccc4ccccc4c3-c3cccc4c3sc3ccccc34)c(-c3ccccc3)c2)cc1 ; CC1(C)c2ccccc2-c2ccc(N(c3ccc(C(c4ccccc4)(c4ccccc4)c4ccccc4)cc3)c3ccc4ccccc4c3-c3cccc4c3sc3ccccc34)cc21 ; CC(C)(C)c1ccc(-c2ccc(N(c3ccc4c(c3)C(C)(C)c3ccccc3-4)c3ccc4ccccc4c3-c3cccc4c3sc3ccccc34)cc2-c2ccccc2)cc1]

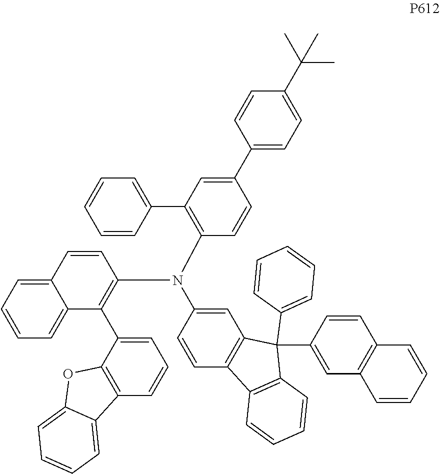 [SMILES: CC(C)(C)c1ccc(-c2ccc(N(c3ccc4c(c3)C(c3ccccc3)(c3ccc5ccccc5c3)c3ccccc3-4)c3ccc4ccccc4c3-c3cccc4c3oc3ccccc34)c(-c3ccccc3)c2)cc1]

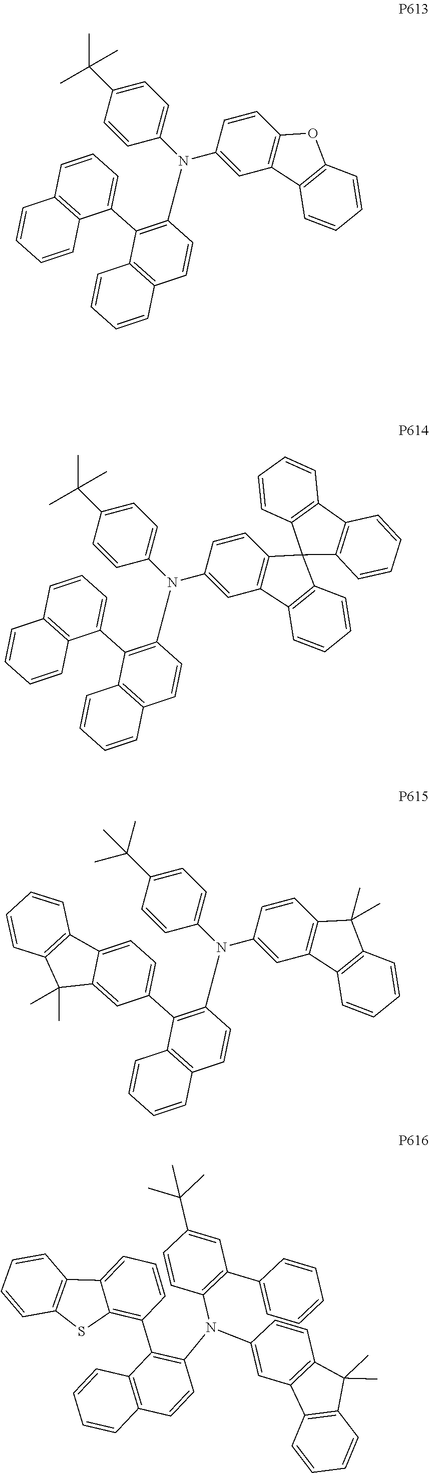 [SMILES: CC(C)(C)c1ccc(N(c2ccc3oc4ccccc4c3c2)c2ccc3ccccc3c2-c2cccc3ccccc23)cc1 ; CC(C)(C)c1ccc(N(c2ccc3c(c2)-c2ccccc2C32c3ccccc3-c3ccccc32)c2ccc3ccccc3c2-c2cccc3ccccc23)cc1 ; CC(C)(C)c1ccc(N(c2ccc3c(c2)-c2ccccc2C3(C)C)c2ccc3ccccc3c2-c2ccc3c(c2)C(C)(C)c2ccccc2-3)cc1 ; CC(C)(C)c1ccc(N(c2ccc3c(c2)-c2ccccc2C3(C)C)c2ccc3ccccc3c2-c2cccc3c2sc2ccccc23)c(-c2ccccc2)c1]

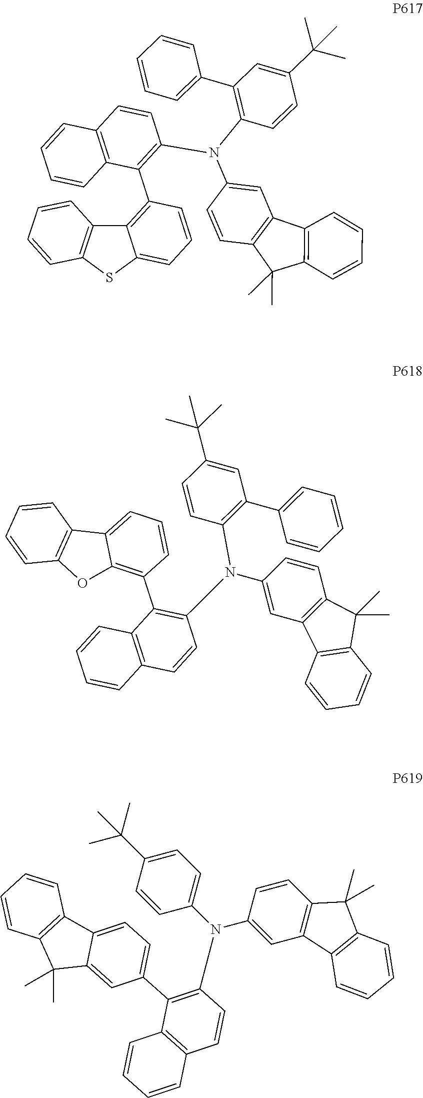

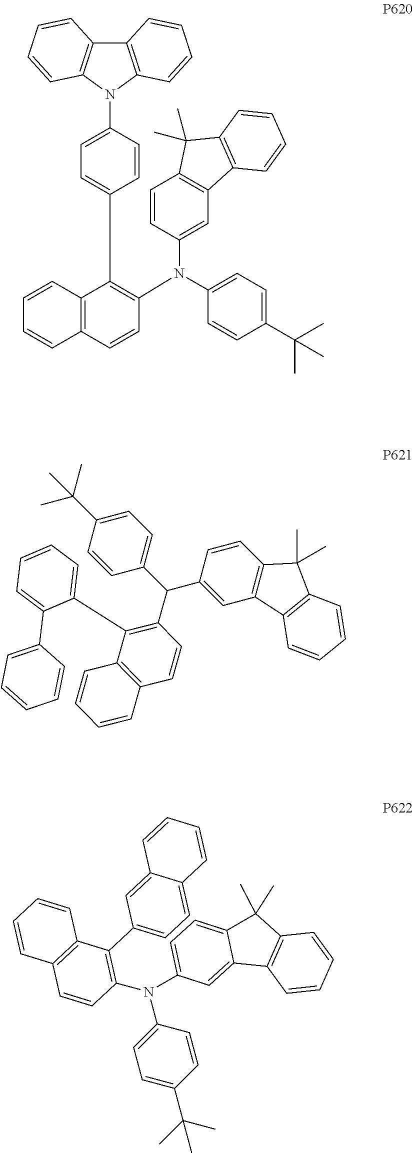

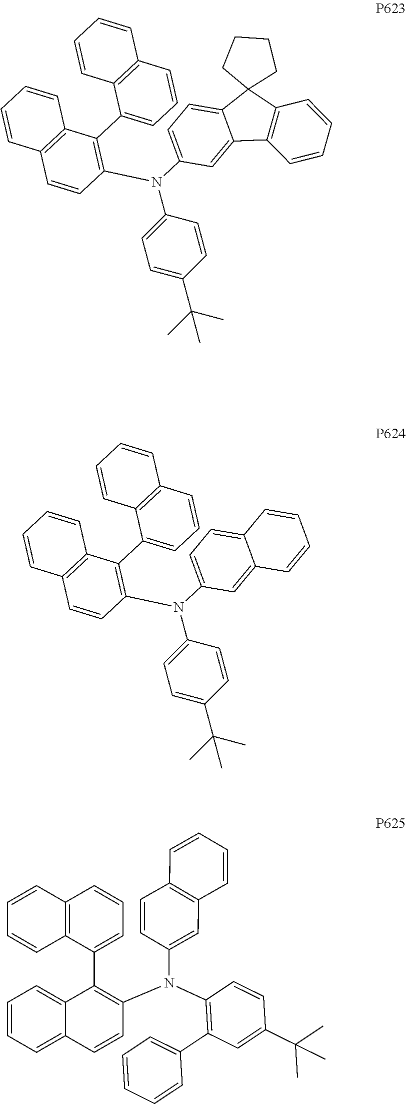 [SMILES: CC(C)(C)c1ccc(N(c2ccc3c(c2)-c2ccccc2C32CCCC2)c2ccc3ccccc3c2-c2cccc3ccccc23)cc1 ; CC(C)(C)c1ccc(N(c2ccc3ccccc3c2)c2ccc3ccccc3c2-c2cccc3ccccc23)cc1 ; CC(C)(C)c1ccc(N(c2ccc3ccccc3c2)c2ccc3ccccc3c2-c2cccc3ccccc23)c(-c2ccccc2)c1]

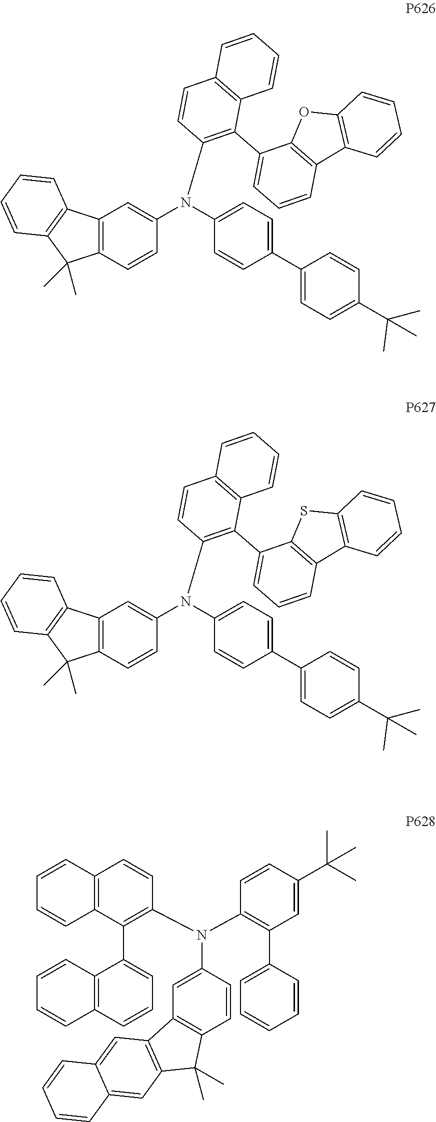 [SMILES: CC(C)(C)c1ccc(-c2ccc(N(c3ccc4c(c3)-c3ccccc3C4(C)C)c3ccc4ccccc4c3-c3cccc4c3oc3ccccc34)cc2)cc1 ; CC(C)(C)c1ccc(-c2ccc(N(c3ccc4c(c3)-c3ccccc3C4(C)C)c3ccc4ccccc4c3-c3cccc4c3sc3ccccc34)cc2)cc1 ; CC(C)(C)c1ccc(N(c2ccc3c(c2)-c2cc4ccccc4cc2C3(C)C)c2ccc3ccccc3c2-c2cccc3ccccc23)c(-c2ccccc2)c1]

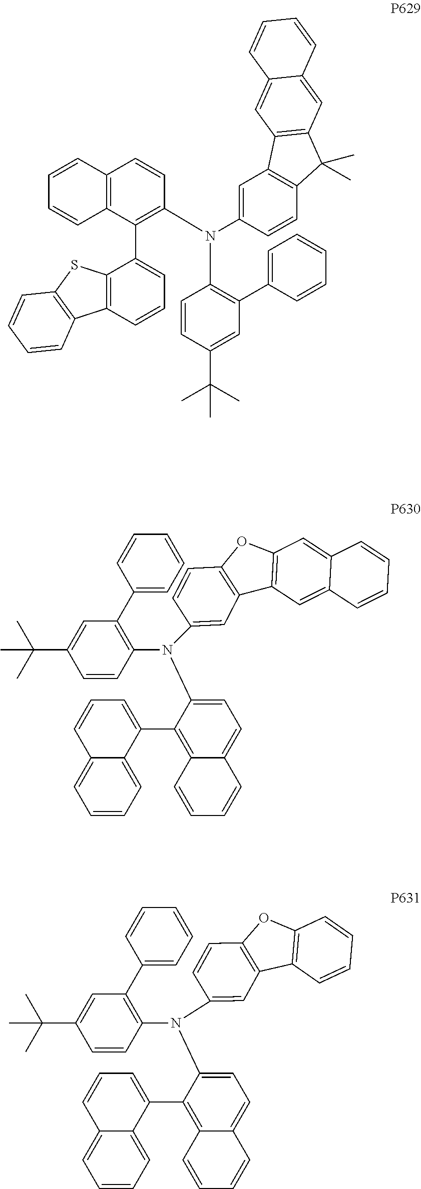

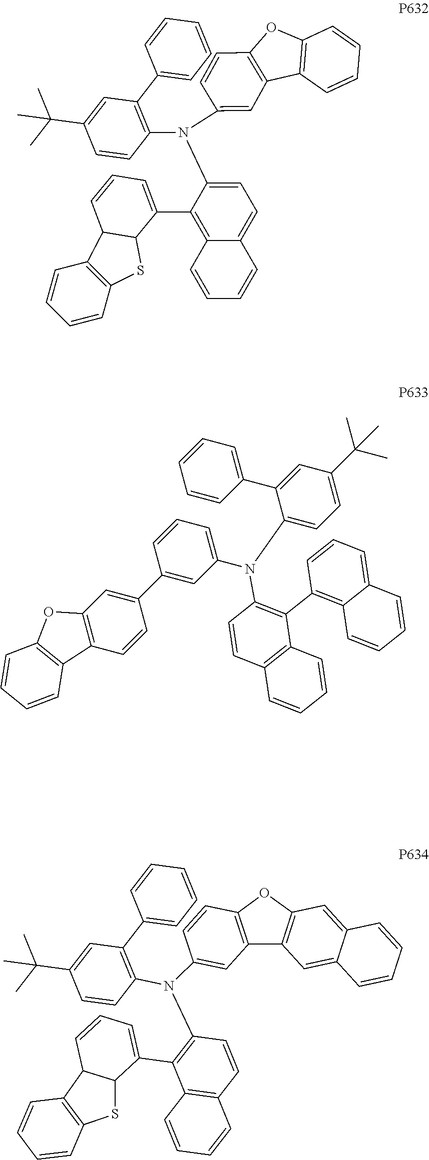 [SMILES: CC(C)(C)c1ccc(N(c2ccc3oc4ccccc4c3c2)c2ccc3ccccc3c2C2=CC=CC3c4ccccc4SC23)c(-c2ccccc2)c1 ; CC(C)(C)c1ccc(N(c2cccc(-c3ccc4c(c3)oc3ccccc34)c2)c2ccc3ccccc3c2-c2cccc3ccccc23)c(-c2ccccc2)c1 ; CC(C)(C)c1ccc(N(c2ccc3oc4cc5ccccc5cc4c3c2)c2ccc3ccccc3c2C2=CC=CC3c4ccccc4SC23)c(-c2ccccc2)c1]

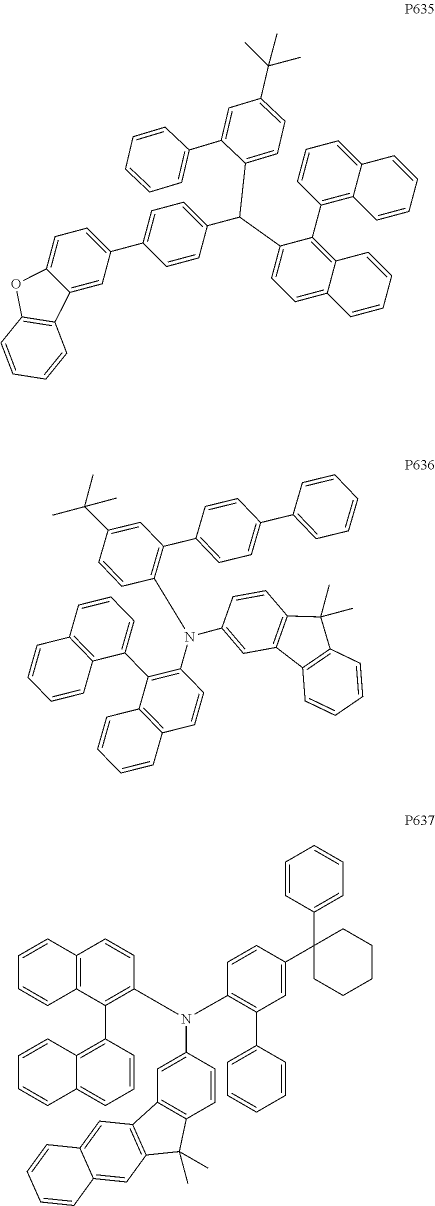 [SMILES: CC(C)(C)c1ccc(C(c2ccc(-c3ccc4oc5ccccc5c4c3)cc2)c2ccc3ccccc3c2-c2cccc3ccccc23)c(-c2ccccc2)c1 ; CC(C)(C)c1ccc(N(c2ccc3c(c2)-c2ccccc2C3(C)C)c2ccc3ccccc3c2-c2cccc3ccccc23)c(-c2ccc(-c3ccccc3)cc2)c1 ; CC1(C)c2ccc(N(c3ccc(C4(c5ccccc5)CCCCC4)cc3-c3ccccc3)c3ccc4ccccc4c3-c3cccc4ccccc34)cc2-c2cc3ccccc3cc21]

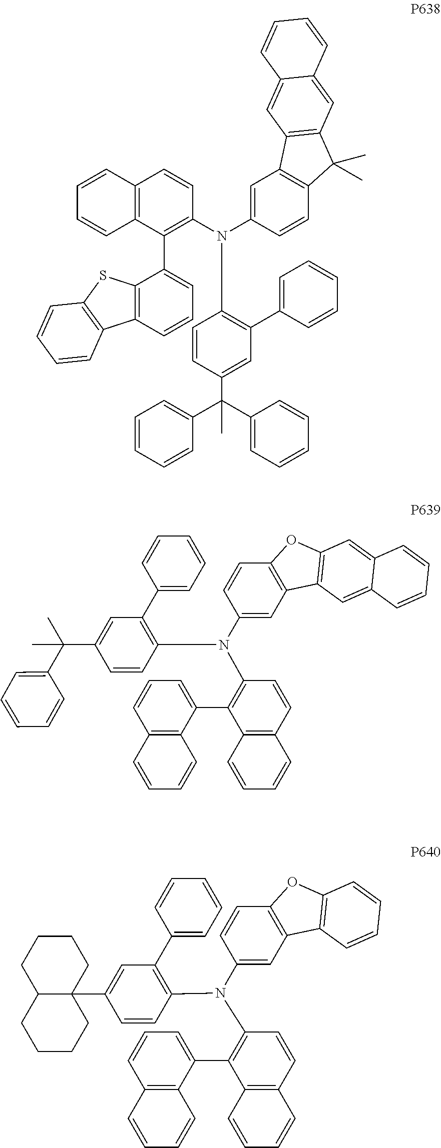 [SMILES: CC1(C)c2ccc(N(c3ccc(C(C)(c4ccccc4)c4ccccc4)cc3-c3ccccc3)c3ccc4ccccc4c3-c3cccc4c3sc3ccccc34)cc2-c2cc3ccccc3cc21 ; CC(C)(c1ccccc1)c1ccc(N(c2ccc3oc4cc5ccccc5cc4c3c2)c2ccc3ccccc3c2-c2cccc3ccccc23)c(-c2ccccc2)c1 ; c1ccc(-c2cc(C34CCCCC3CCCC4)ccc2N(c2ccc3oc4ccccc4c3c2)c2ccc3ccccc3c2-c2cccc3ccccc23)cc1]

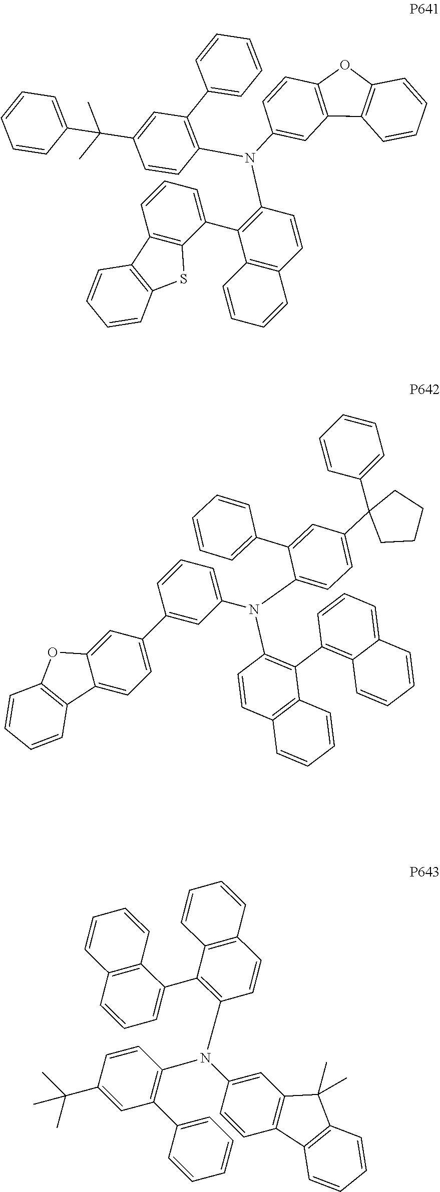 [SMILES: CC(C)(c1ccccc1)c1ccc(N(c2ccc3oc4ccccc4c3c2)c2ccc3ccccc3c2-c2cccc3c2sc2ccccc23)c(-c2ccccc2)c1 ; c1ccc(-c2cc(C3(c4ccccc4)CCCC3)ccc2N(c2cccc(-c3ccc4c(c3)oc3ccccc34)c2)c2ccc3ccccc3c2-c2cccc3ccccc23)cc1 ; CC(C)(C)c1ccc(N(c2ccc3c(c2)C(C)(C)c2ccccc2-3)c2ccc3ccccc3c2-c2cccc3ccccc23)c(-c2ccccc2)c1]

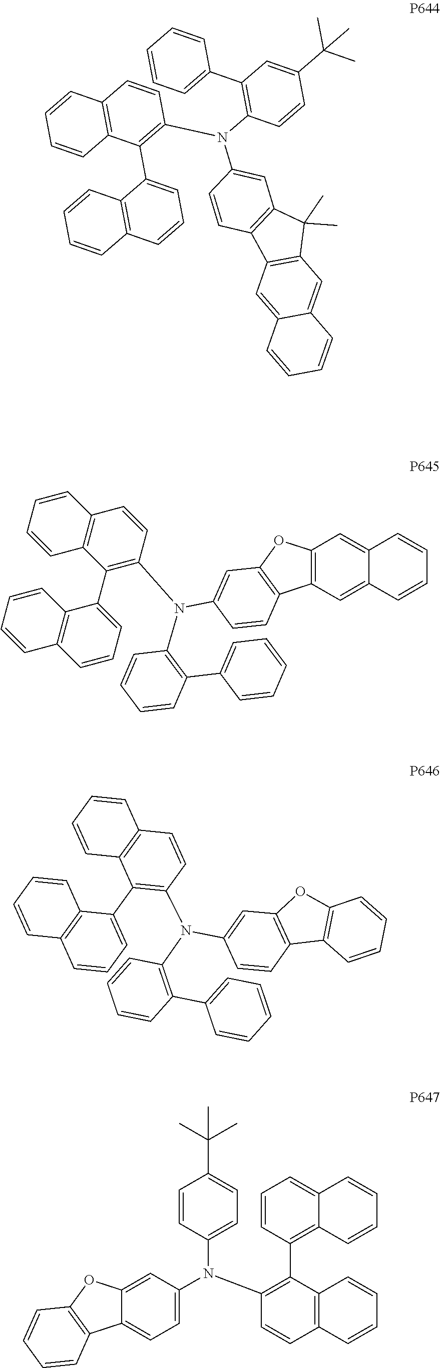 [SMILES: CC(C)(C)c1ccc(N(c2ccc3c(c2)C(C)(C)c2cc4ccccc4cc2-3)c2ccc3ccccc3c2-c2cccc3ccccc23)c(-c2ccccc2)c1 ; c1ccc(-c2ccccc2N(c2ccc3c(c2)oc2cc4ccccc4cc23)c2ccc3ccccc3c2-c2cccc3ccccc23)cc1 ; c1ccc(-c2ccccc2N(c2ccc3c(c2)oc2ccccc23)c2ccc3ccccc3c2-c2cccc3ccccc23)cc1 ; CC(C)(C)c1ccc(N(c2ccc3c(c2)oc2ccccc23)c2ccc3ccccc3c2-c2cccc3ccccc23)cc1]

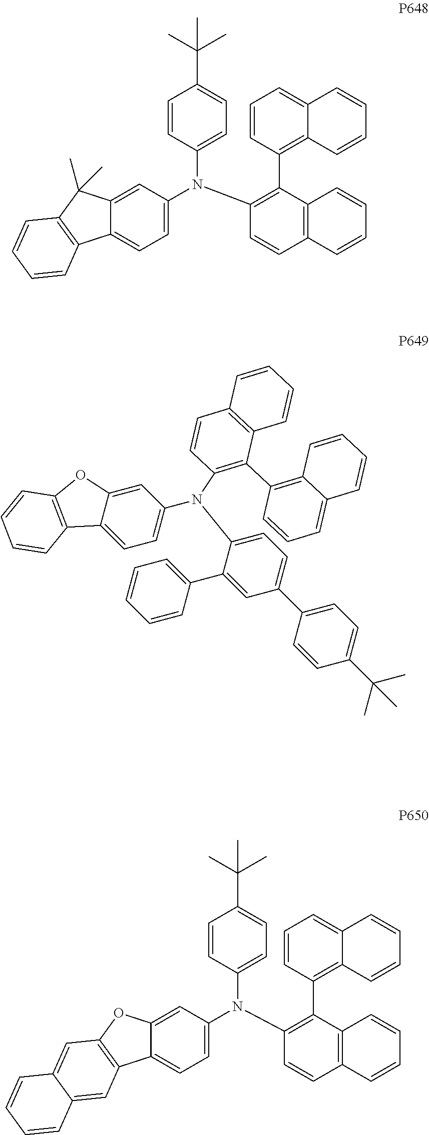 [SMILES: CC(C)(C)c1ccc(N(c2ccc3c(c2)C(C)(C)c2ccccc2-3)c2ccc3ccccc3c2-c2cccc3ccccc23)cc1 ; CC(C)(C)c1ccc(-c2ccc(N(c3ccc4c(c3)oc3ccccc34)c3ccc4ccccc4c3-c3cccc4ccccc34)c(-c3ccccc3)c2)cc1 ; CC(C)(C)c1ccc(N(c2ccc3c(c2)oc2cc4ccccc4cc23)c2ccc3ccccc3c2-c2cccc3ccccc23)cc1]

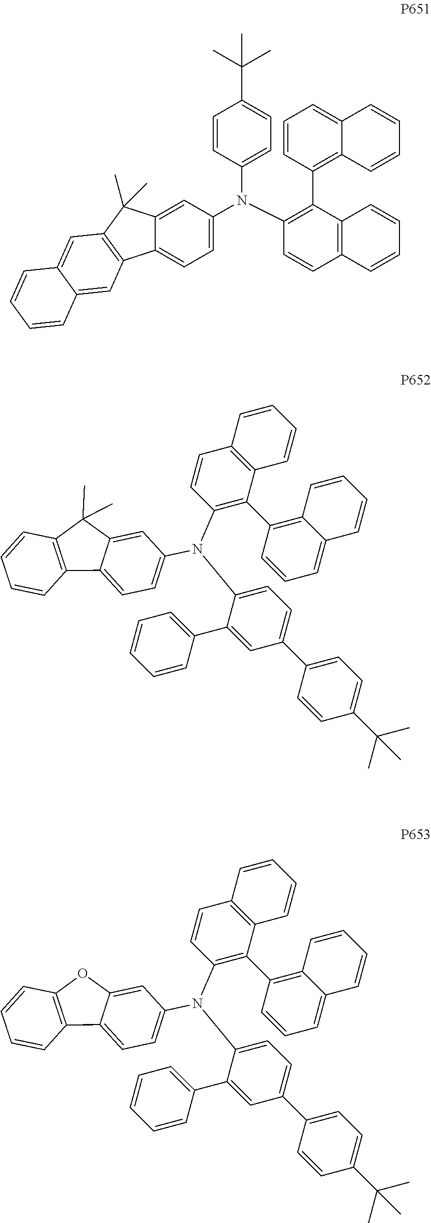 [SMILES: CC(C)(C)c1ccc(N(c2ccc3c(c2)C(C)(C)c2cc4ccccc4cc2-3)c2ccc3ccccc3c2-c2cccc3ccccc23)cc1 ; CC(C)(C)c1ccc(-c2ccc(N(c3ccc4c(c3)C(C)(C)c3ccccc3-4)c3ccc4ccccc4c3-c3cccc4ccccc34)c(-c3ccccc3)c2)cc1 ; CC(C)(C)c1ccc(-c2ccc(N(c3ccc4c(c3)oc3ccccc34)c3ccc4ccccc4c3-c3cccc4ccccc34)c(-c3ccccc3)c2)cc1]

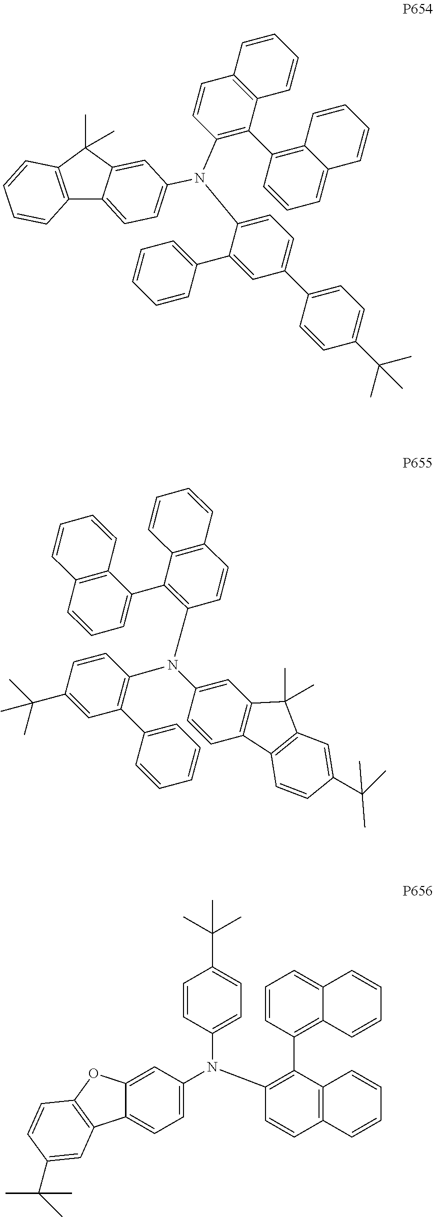

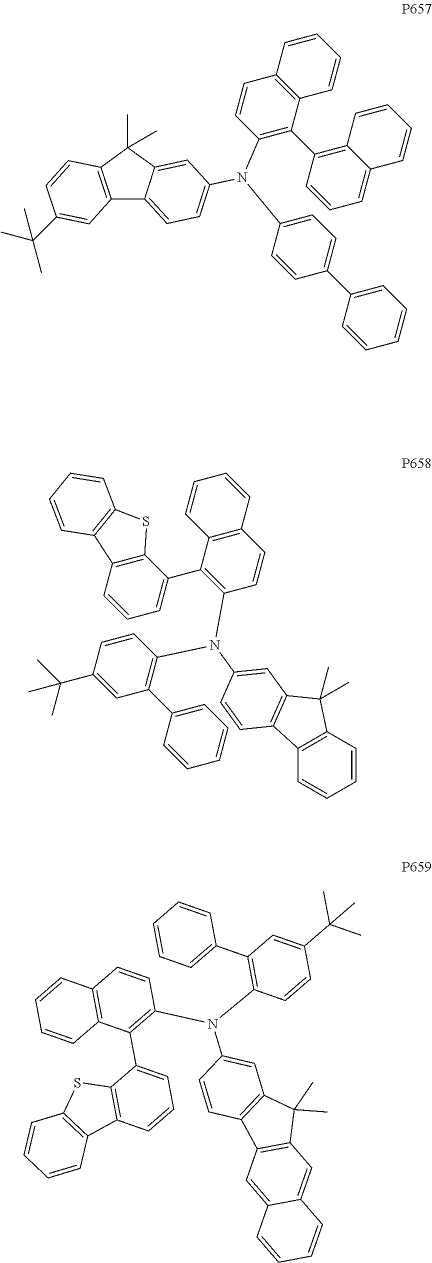 [SMILES: CC(C)(C)c1ccc2c(c1)-c1ccc(N(c3ccc(-c4ccccc4)cc3)c3ccc4ccccc4c3-c3cccc4ccccc34)cc1C2(C)C ; CC(C)(C)c1ccc(N(c2ccc3c(c2)C(C)(C)c2ccccc2-3)c2ccc3ccccc3c2-c2cccc3c2sc2ccccc23)c(-c2ccccc2)c1 ; CC(C)(C)c1ccc(N(c2ccc3c(c2)C(C)(C)c2cc4ccccc4cc2-3)c2ccc3ccccc3c2-c2cccc3c2sc2ccccc23)c(-c2ccccc2)c1]

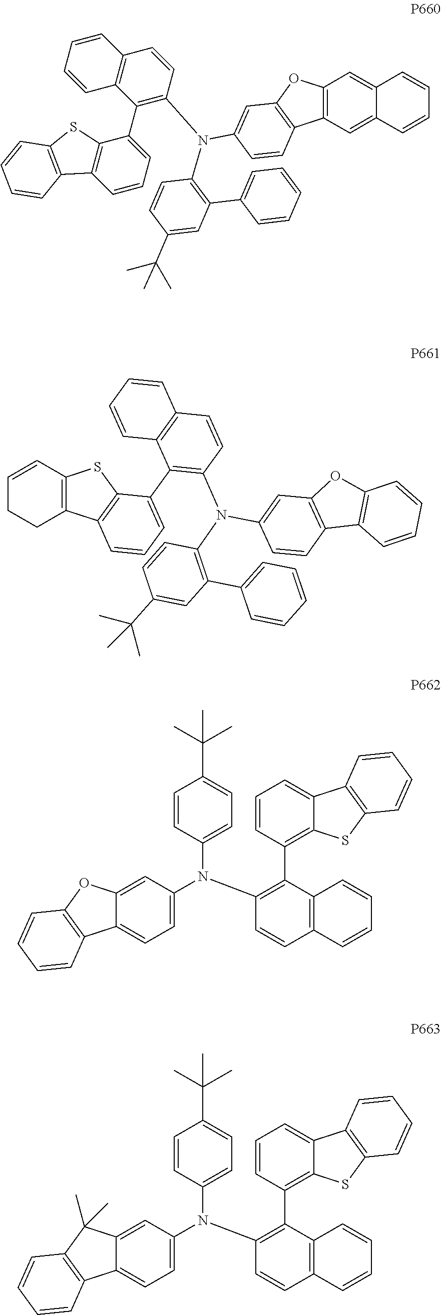 [SMILES: CC(C)(C)c1ccc(N(c2ccc3c(c2)oc2cc4ccccc4cc23)c2ccc3ccccc3c2-c2cccc3c2sc2ccccc23)c(-c2ccccc2)c1 ; CC(C)(C)c1ccc(N(c2ccc3c(c2)oc2ccccc23)c2ccc3ccccc3c2-c2cccc3c4c(sc23)C=CCC4)c(-c2ccccc2)c1 ; CC(C)(C)c1ccc(N(c2ccc3c(c2)oc2ccccc23)c2ccc3ccccc3c2-c2cccc3c2sc2ccccc23)cc1 ; CC(C)(C)c1ccc(N(c2ccc3c(c2)C(C)(C)c2ccccc2-3)c2ccc3ccccc3c2-c2cccc3c2sc2ccccc23)cc1]

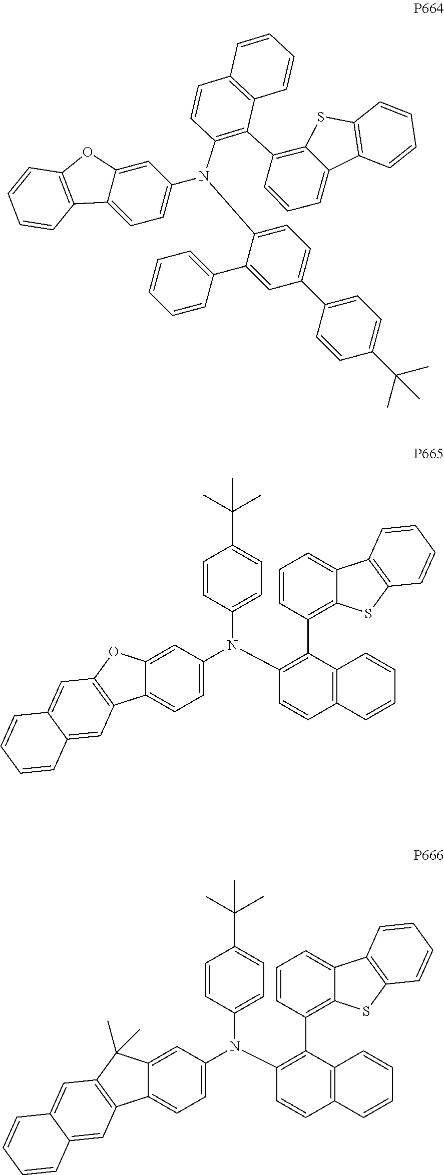 [SMILES: CC(C)(C)c1ccc(-c2ccc(N(c3ccc4c(c3)oc3ccccc34)c3ccc4ccccc4c3-c3cccc4c3sc3ccccc34)c(-c3ccccc3)c2)cc1 ; CC(C)(C)c1ccc(N(c2ccc3c(c2)oc2cc4ccccc4cc23)c2ccc3ccccc3c2-c2cccc3c2sc2ccccc23)cc1 ; CC(C)(C)c1ccc(N(c2ccc3c(c2)C(C)(C)c2cc4ccccc4cc2-3)c2ccc3ccccc3c2-c2cccc3c2sc2ccccc23)cc1]

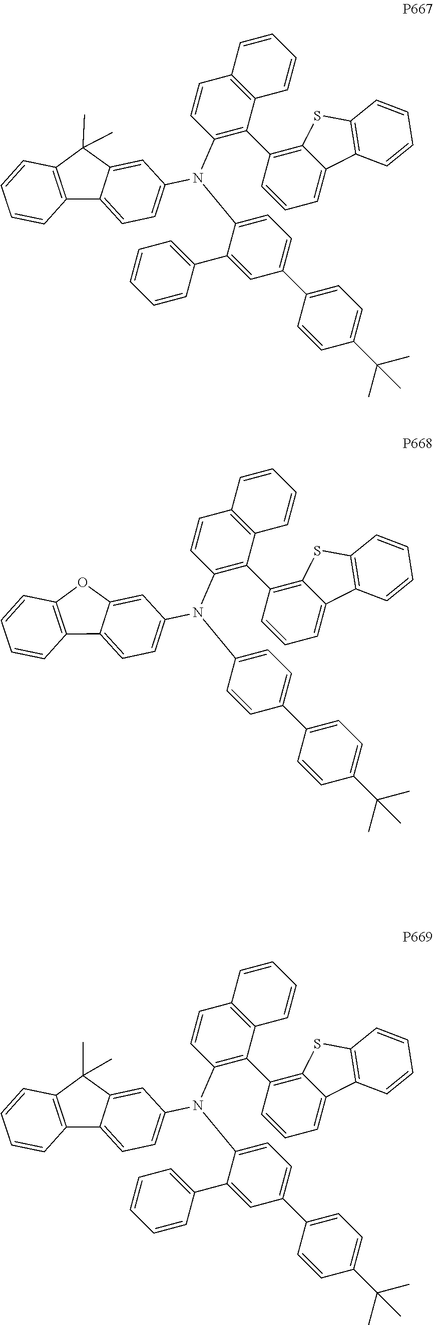 [SMILES: CC(C)(C)c1ccc(-c2ccc(N(c3ccc4c(c3)C(C)(C)c3ccccc3-4)c3ccc4ccccc4c3-c3cccc4c3sc3ccccc34)c(-c3ccccc3)c2)cc1 ; CC(C)(C)c1ccc(-c2ccc(N(c3ccc4c(c3)oc3ccccc34)c3ccc4ccccc4c3-c3cccc4c3sc3ccccc34)cc2)cc1 ; CC(C)(C)c1ccc(-c2ccc(N(c3ccc4c(c3)C(C)(C)c3ccccc3-4)c3ccc4ccccc4c3-c3cccc4c3sc3ccccc34)c(-c3ccccc3)c2)cc1]

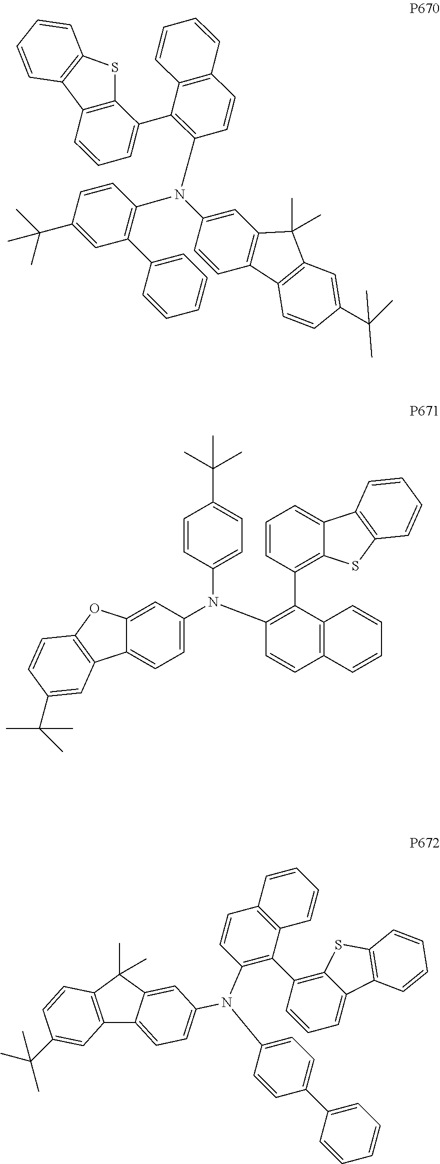

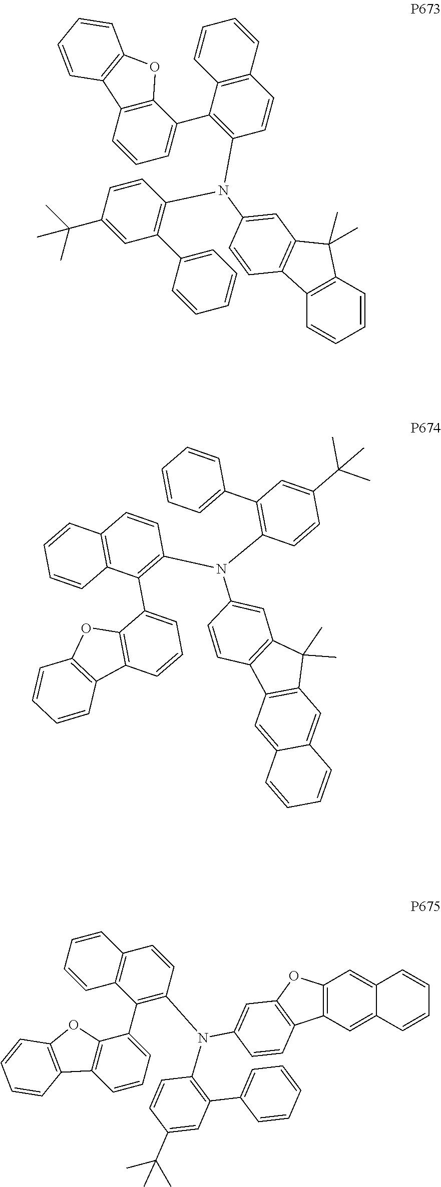 [SMILES: CC(C)(C)c1ccc(N(c2ccc3c(c2)C(C)(C)c2ccccc2-3)c2ccc3ccccc3c2-c2cccc3c2oc2ccccc23)c(-c2ccccc2)c1 ; CC(C)(C)c1ccc(N(c2ccc3c(c2)C(C)(C)c2cc4ccccc4cc2-3)c2ccc3ccccc3c2-c2cccc3c2oc2ccccc23)c(-c2ccccc2)c1 ; CC(C)(C)c1ccc(N(c2ccc3c(c2)oc2cc4ccccc4cc23)c2ccc3ccccc3c2-c2cccc3c2oc2ccccc23)c(-c2ccccc2)c1]

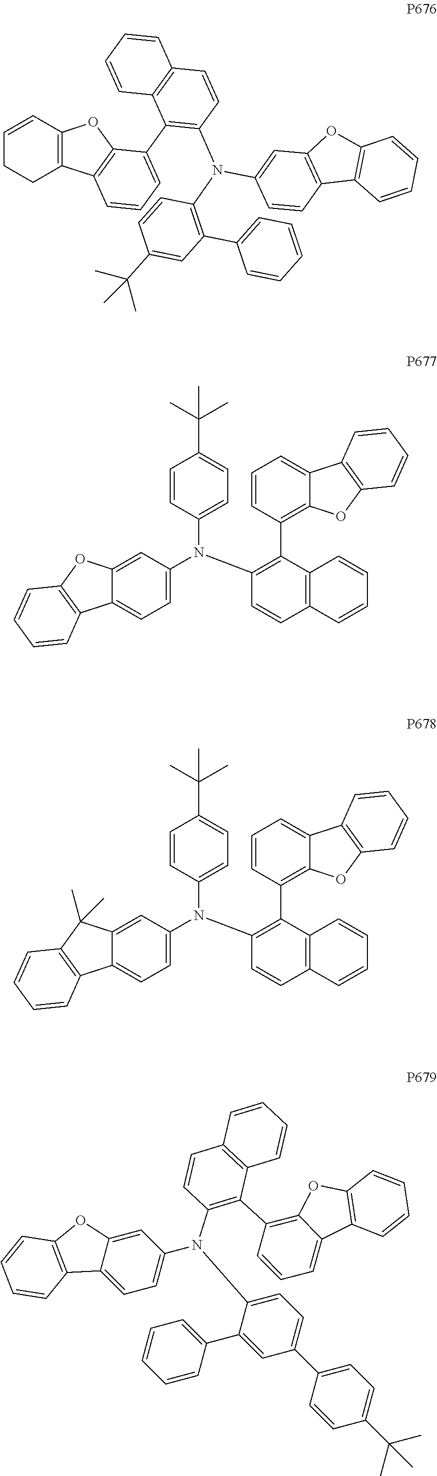 [SMILES: CC(C)(C)c1ccc(N(c2ccc3c(c2)oc2ccccc23)c2ccc3ccccc3c2-c2cccc3c4c(oc23)C=CCC4)c(-c2ccccc2)c1 ; CC(C)(C)c1ccc(N(c2ccc3c(c2)oc2ccccc23)c2ccc3ccccc3c2-c2cccc3c2oc2ccccc23)cc1 ; CC(C)(C)c1ccc(N(c2ccc3c(c2)C(C)(C)c2ccccc2-3)c2ccc3ccccc3c2-c2cccc3c2oc2ccccc23)cc1 ; CC(C)(C)c1ccc(-c2ccc(N(c3ccc4c(c3)oc3ccccc34)c3ccc4ccccc4c3-c3cccc4c3oc3ccccc34)c(-c3ccccc3)c2)cc1]

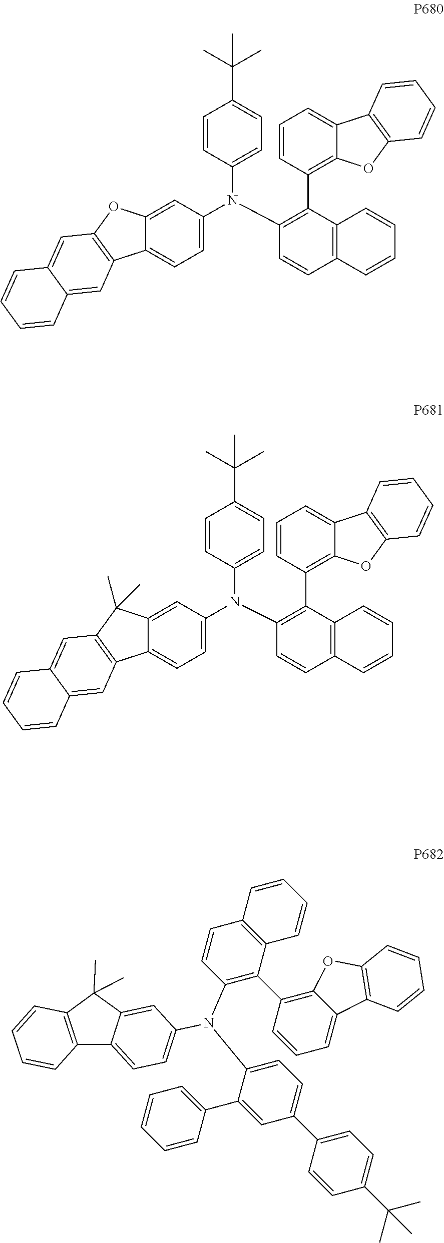 [SMILES: CC(C)(C)c1ccc(N(c2ccc3c(c2)oc2cc4ccccc4cc23)c2ccc3ccccc3c2-c2cccc3c2oc2ccccc23)cc1 ; CC(C)(C)c1ccc(N(c2ccc3c(c2)C(C)(C)c2cc4ccccc4cc2-3)c2ccc3ccccc3c2-c2cccc3c2oc2ccccc23)cc1 ; CC(C)(C)c1ccc(-c2ccc(N(c3ccc4c(c3)C(C)(C)c3ccccc3-4)c3ccc4ccccc4c3-c3cccc4c3oc3ccccc34)c(-c3ccccc3)c2)cc1]

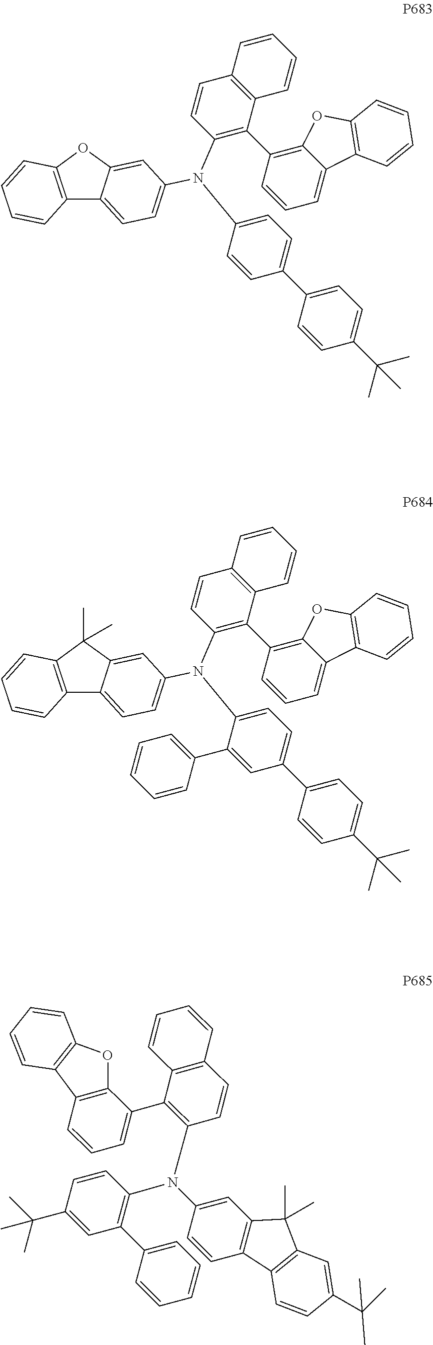 [SMILES: CC(C)(C)c1ccc(-c2ccc(N(c3ccc4c(c3)oc3ccccc34)c3ccc4ccccc4c3-c3cccc4c3oc3ccccc34)cc2)cc1 ; CC(C)(C)c1ccc(-c2ccc(N(c3ccc4c(c3)C(C)(C)c3ccccc3-4)c3ccc4ccccc4c3-c3cccc4c3oc3ccccc34)c(-c3ccccc3)c2)cc1 ; CC(C)(C)c1ccc(N(c2ccc3c(c2)C(C)(C)c2cc(C(C)(C)C)ccc2-3)c2ccc3ccccc3c2-c2cccc3c2oc2ccccc23)c(-c2ccccc2)c1]

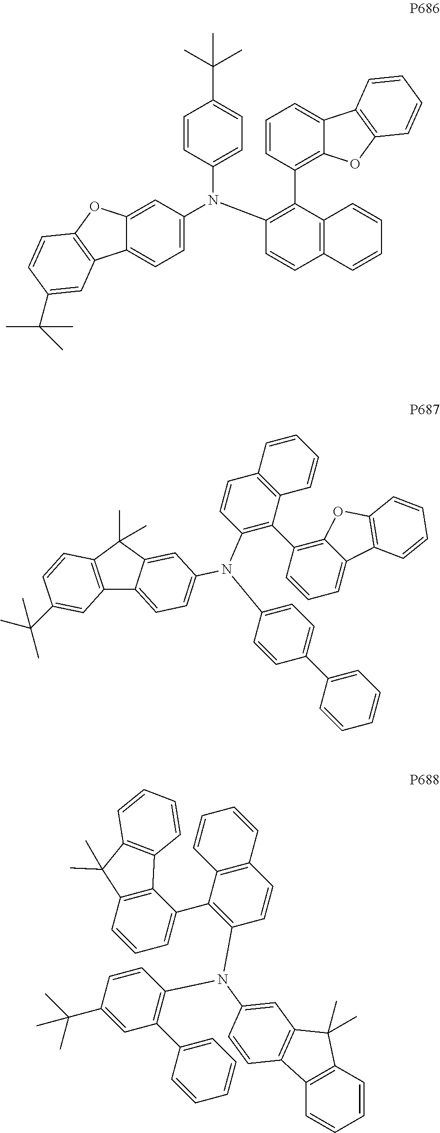 [SMILES: CC(C)(C)c1ccc(N(c2ccc3c(c2)oc2ccc(C(C)(C)C)cc23)c2ccc3ccccc3c2-c2cccc3c2oc2ccccc23)cc1 ; CC(C)(C)c1ccc2c(c1)-c1ccc(N(c3ccc(-c4ccccc4)cc3)c3ccc4ccccc4c3-c3cccc4c3oc3ccccc34)cc1C2(C)C ; CC(C)(C)c1ccc(N(c2ccc3c(c2)C(C)(C)c2ccccc2-3)c2ccc3ccccc3c2-c2cccc3c2-c2ccccc2C3(C)C)c(-c2ccccc2)c1]

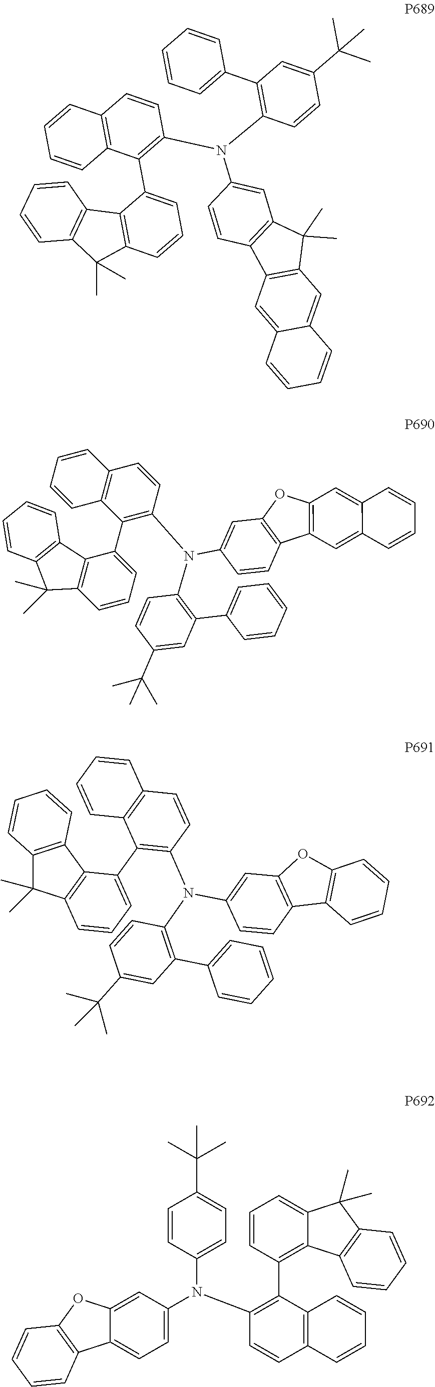

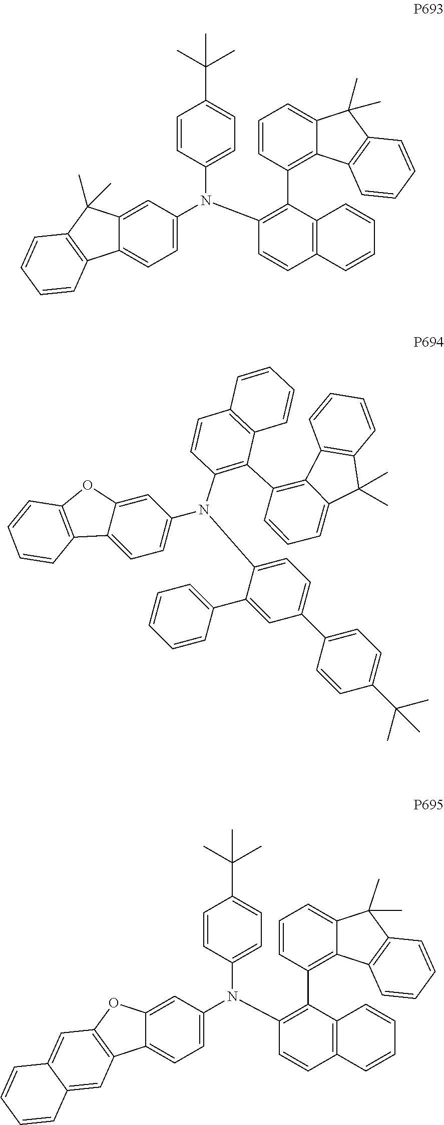 [SMILES: CC(C)(C)c1ccc(N(c2ccc3c(c2)C(C)(C)c2ccccc2-3)c2ccc3ccccc3c2-c2cccc3c2-c2ccccc2C3(C)C)cc1 ; CC(C)(C)c1ccc(-c2ccc(N(c3ccc4c(c3)oc3ccccc34)c3ccc4ccccc4c3-c3cccc4c3-c3ccccc3C4(C)C)c(-c3ccccc3)c2)cc1 ; CC(C)(C)c1ccc(N(c2ccc3c(c2)oc2cc4ccccc4cc23)c2ccc3ccccc3c2-c2cccc3c2-c2ccccc2C3(C)C)cc1]

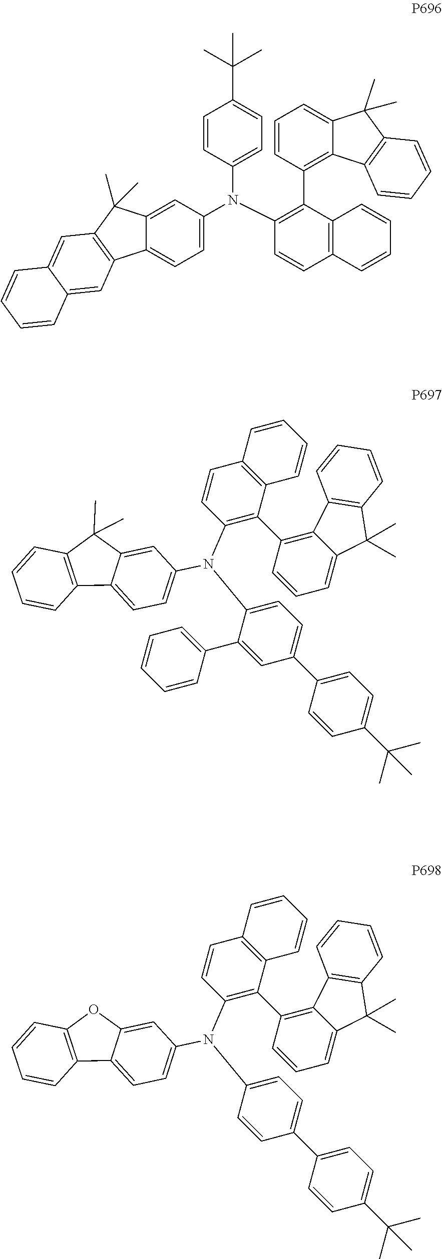 [SMILES: CC(C)(C)c1ccc(N(c2ccc3c(c2)C(C)(C)c2cc4ccccc4cc2-3)c2ccc3ccccc3c2-c2cccc3c2-c2ccccc2C3(C)C)cc1 ; CC(C)(C)c1ccc(-c2ccc(N(c3ccc4c(c3)C(C)(C)c3ccccc3-4)c3ccc4ccccc4c3-c3cccc4c3-c3ccccc3C4(C)C)c(-c3ccccc3)c2)cc1 ; CC(C)(C)c1ccc(-c2ccc(N(c3ccc4c(c3)oc3ccccc34)c3ccc4ccccc4c3-c3cccc4c3-c3ccccc3C4(C)C)cc2)cc1]

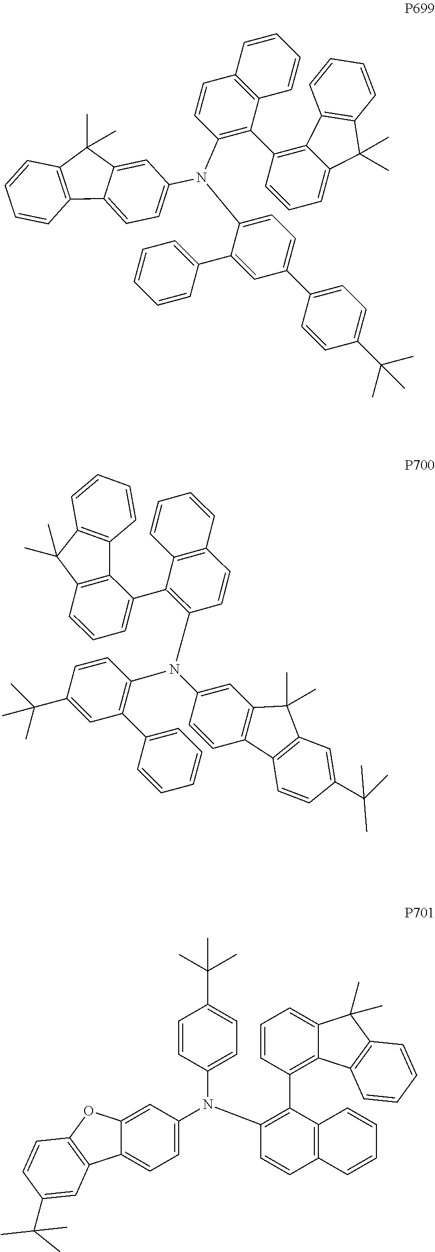

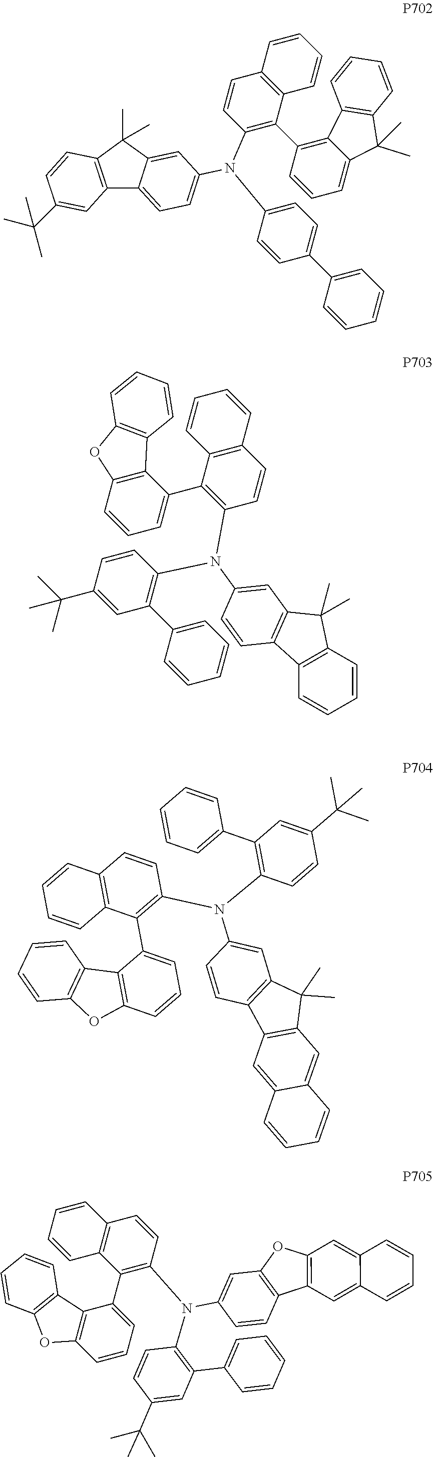

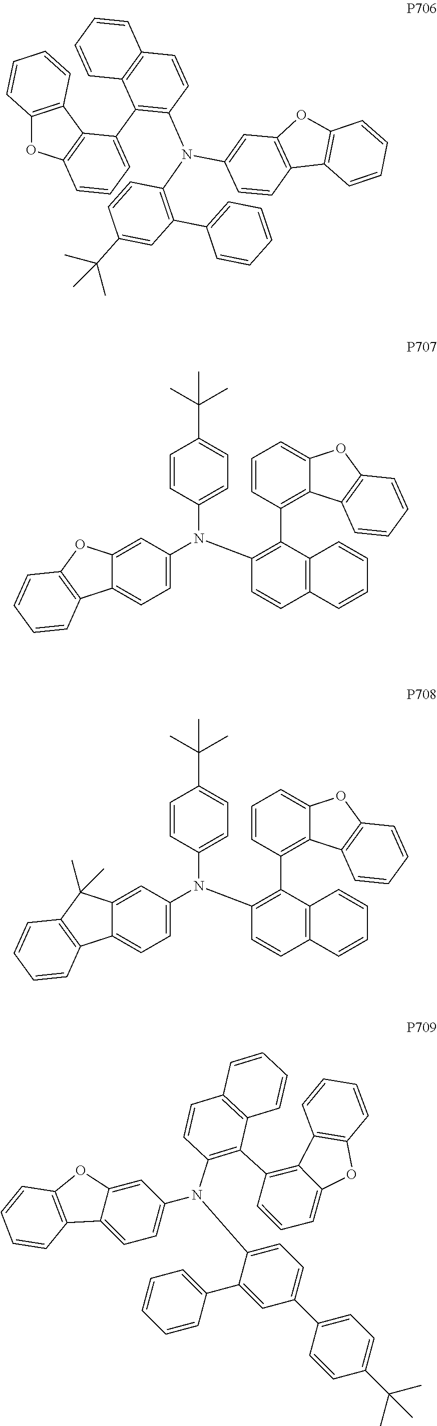 [SMILES: CC(C)(C)c1ccc(N(c2ccc3c(c2)oc2ccccc23)c2ccc3ccccc3c2-c2cccc3oc4ccccc4c23)c(-c2ccccc2)c1 ; CC(C)(C)c1ccc(N(c2ccc3c(c2)oc2ccccc23)c2ccc3ccccc3c2-c2cccc3oc4ccccc4c23)cc1 ; CC(C)(C)c1ccc(N(c2ccc3c(c2)C(C)(C)c2ccccc2-3)c2ccc3ccccc3c2-c2cccc3oc4ccccc4c23)cc1 ; CC(C)(C)c1ccc(-c2ccc(N(c3ccc4c(c3)oc3ccccc34)c3ccc4ccccc4c3-c3cccc4oc5ccccc5c34)c(-c3ccccc3)c2)cc1]

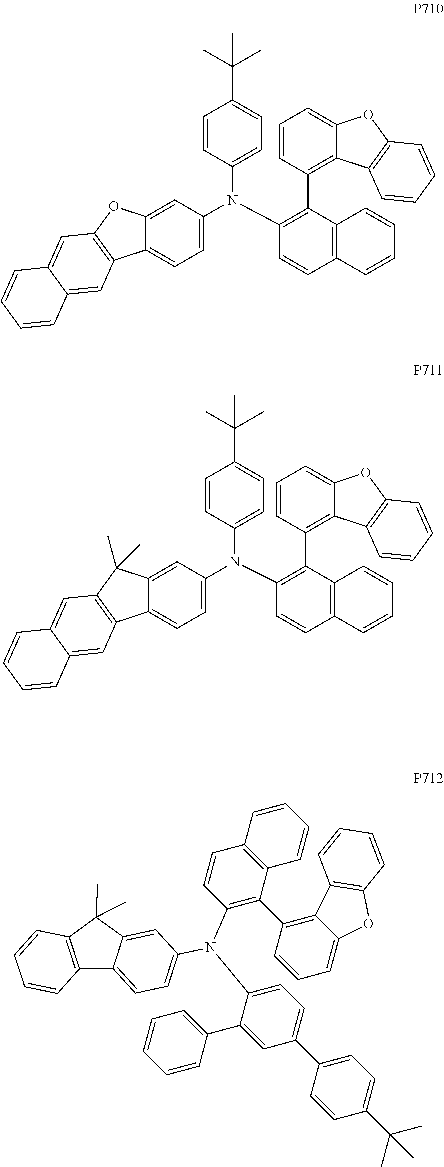 [SMILES: CC(C)(C)c1ccc(N(c2ccc3c(c2)oc2cc4ccccc4cc23)c2ccc3ccccc3c2-c2cccc3oc4ccccc4c23)cc1 ; CC(C)(C)c1ccc(N(c2ccc3c(c2)C(C)(C)c2cc4ccccc4cc2-3)c2ccc3ccccc3c2-c2cccc3oc4ccccc4c23)cc1 ; CC(C)(C)c1ccc(-c2ccc(N(c3ccc4c(c3)C(C)(C)c3ccccc3-4)c3ccc4ccccc4c3-c3cccc4oc5ccccc5c34)c(-c3ccccc3)c2)cc1]

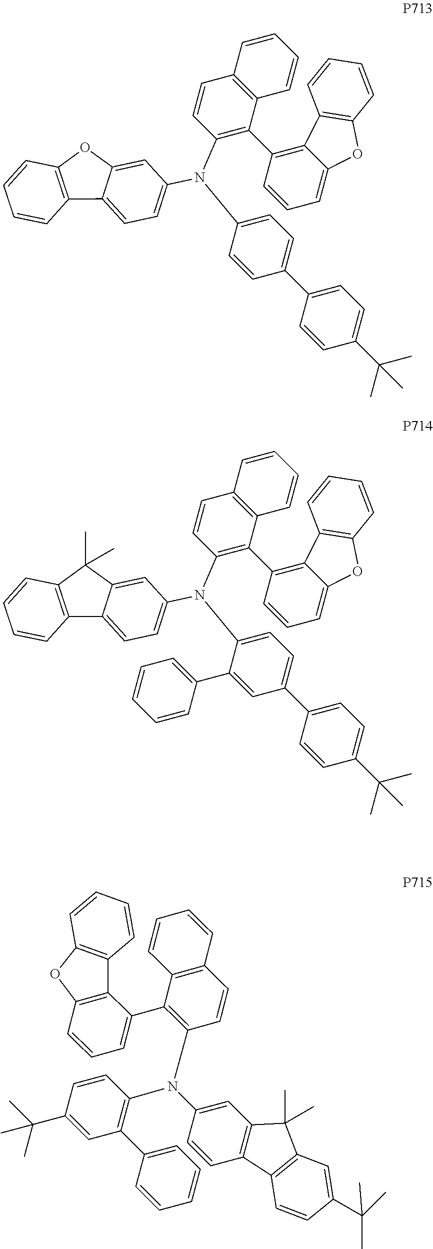

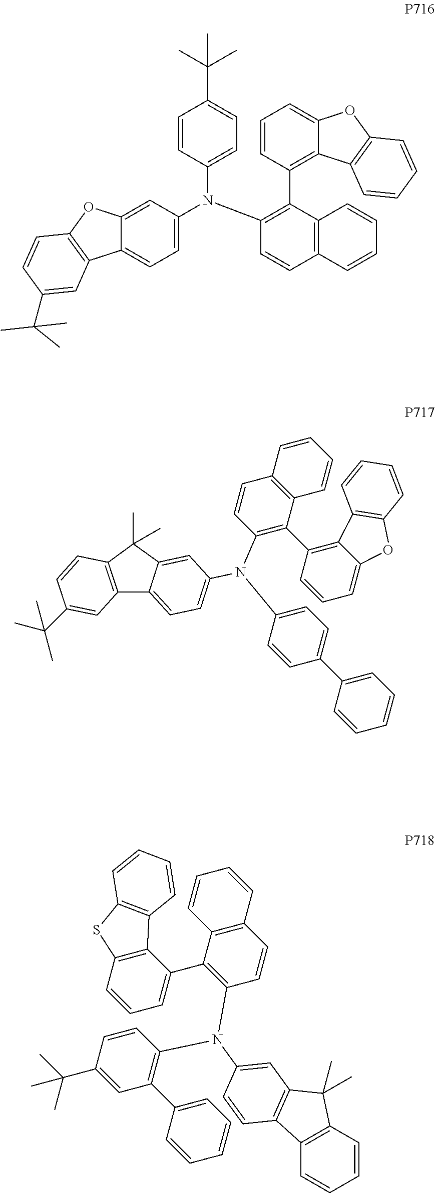 [SMILES: CC(C)(C)c1ccc(N(c2ccc3c(c2)oc2ccc(C(C)(C)C)cc23)c2ccc3ccccc3c2-c2cccc3oc4ccccc4c23)cc1 ; CC(C)(C)c1ccc2c(c1)-c1ccc(N(c3ccc(-c4ccccc4)cc3)c3ccc4ccccc4c3-c3cccc4oc5ccccc5c34)cc1C2(C)C ; CC(C)(C)c1ccc(N(c2ccc3c(c2)C(C)(C)c2ccccc2-3)c2ccc3ccccc3c2-c2cccc3sc4ccccc4c23)c(-c2ccccc2)c1]

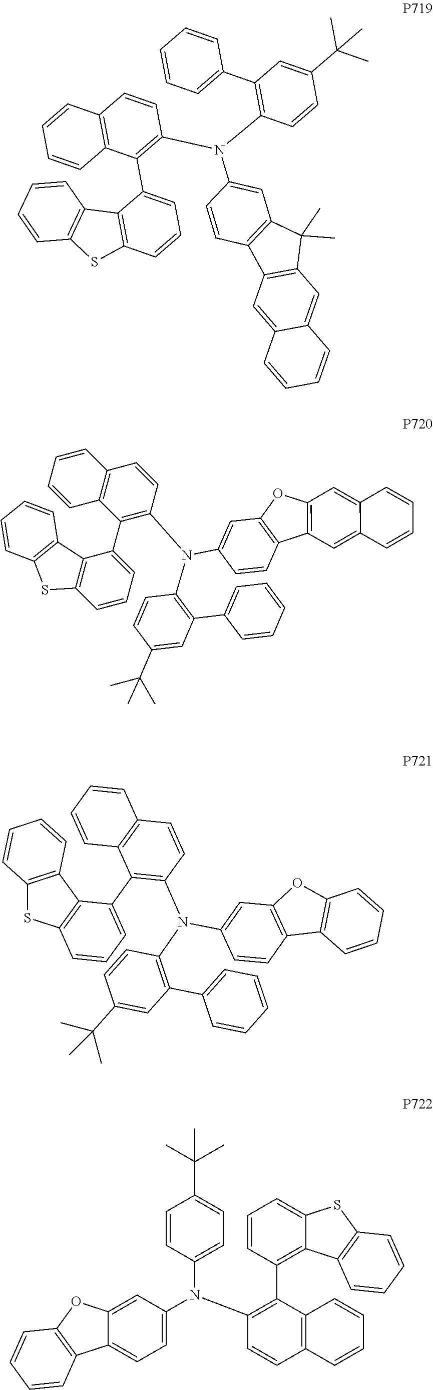 [SMILES: CC(C)(C)c1ccc(N(c2ccc3c(c2)C(C)(C)c2cc4ccccc4cc2-3)c2ccc3ccccc3c2-c2cccc3sc4ccccc4c23)c(-c2ccccc2)c1 ; CC(C)(C)c1ccc(N(c2ccc3c(c2)oc2cc4ccccc4cc23)c2ccc3ccccc3c2-c2cccc3sc4ccccc4c23)c(-c2ccccc2)c1 ; CC(C)(C)c1ccc(N(c2ccc3c(c2)oc2ccccc23)c2ccc3ccccc3c2-c2cccc3sc4ccccc4c23)c(-c2ccccc2)c1 ; CC(C)(C)c1ccc(N(c2ccc3c(c2)oc2ccccc23)c2ccc3ccccc3c2-c2cccc3sc4ccccc4c23)cc1]

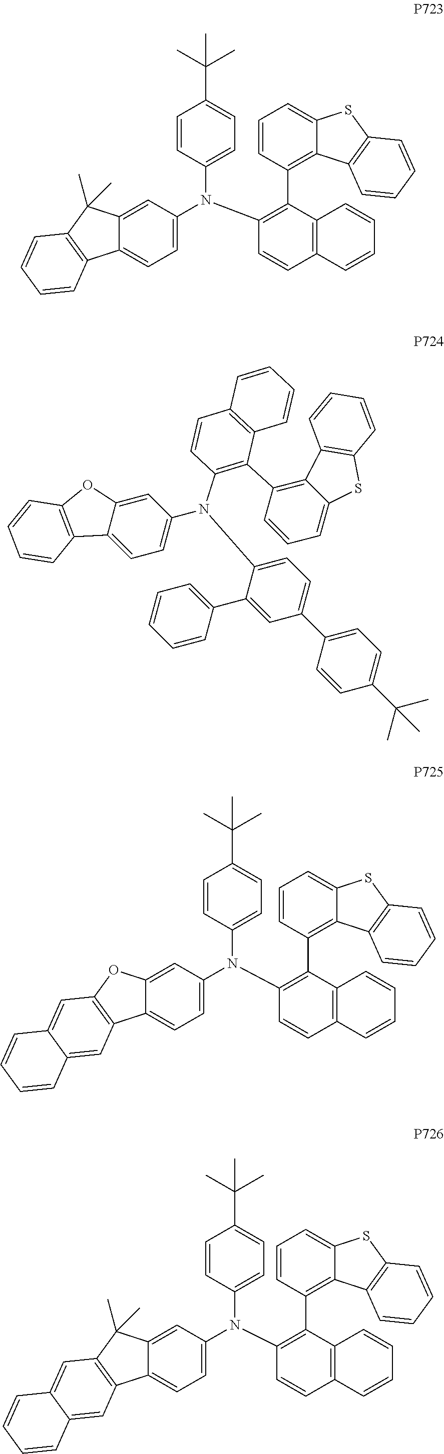 [SMILES: CC(C)(C)c1ccc(N(c2ccc3c(c2)C(C)(C)c2ccccc2-3)c2ccc3ccccc3c2-c2cccc3sc4ccccc4c23)cc1 ; CC(C)(C)c1ccc(-c2ccc(N(c3ccc4c(c3)oc3ccccc34)c3ccc4ccccc4c3-c3cccc4sc5ccccc5c34)c(-c3ccccc3)c2)cc1 ; CC(C)(C)c1ccc(N(c2ccc3c(c2)oc2cc4ccccc4cc23)c2ccc3ccccc3c2-c2cccc3sc4ccccc4c23)cc1 ; CC(C)(C)c1ccc(N(c2ccc3c(c2)C(C)(C)c2cc4ccccc4cc2-3)c2ccc3ccccc3c2-c2cccc3sc4ccccc4c23)cc1]

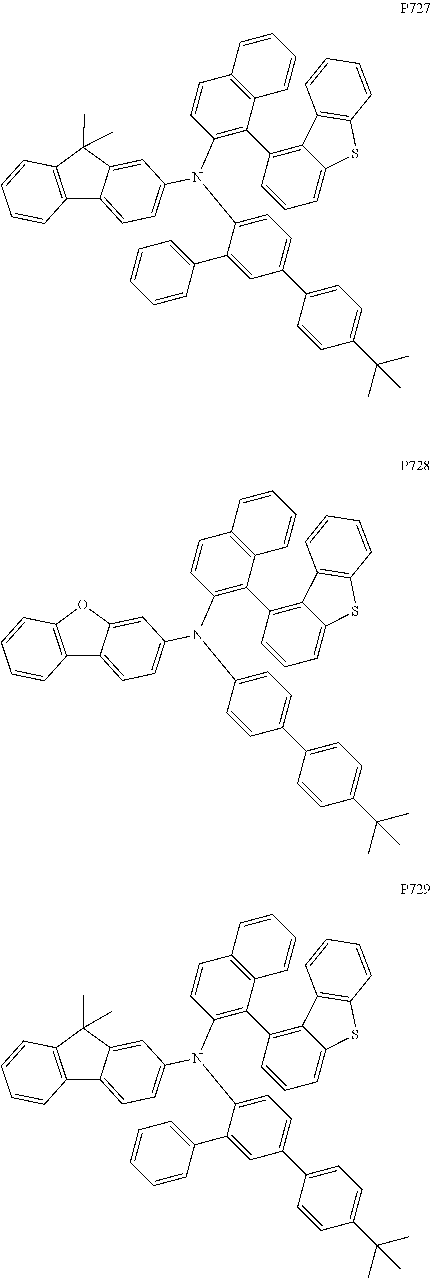 [SMILES: CC(C)(C)c1ccc(-c2ccc(N(c3ccc4c(c3)C(C)(C)c3ccccc3-4)c3ccc4ccccc4c3-c3cccc4sc5ccccc5c34)c(-c3ccccc3)c2)cc1 ; CC(C)(C)c1ccc(-c2ccc(N(c3ccc4c(c3)oc3ccccc34)c3ccc4ccccc4c3-c3cccc4sc5ccccc5c34)cc2)cc1 ; CC(C)(C)c1ccc(-c2ccc(N(c3ccc4c(c3)C(C)(C)c3ccccc3-4)c3ccc4ccccc4c3-c3cccc4sc5ccccc5c34)c(-c3ccccc3)c2)cc1]

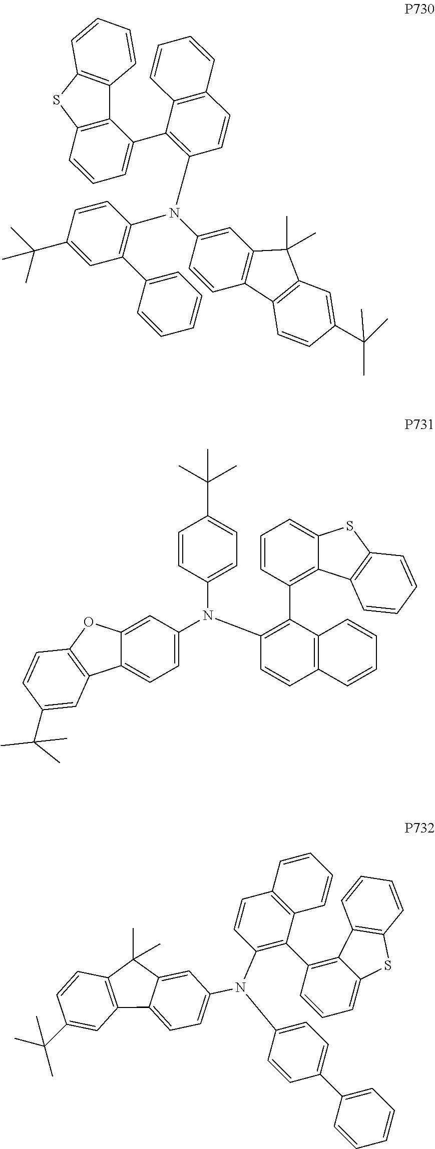

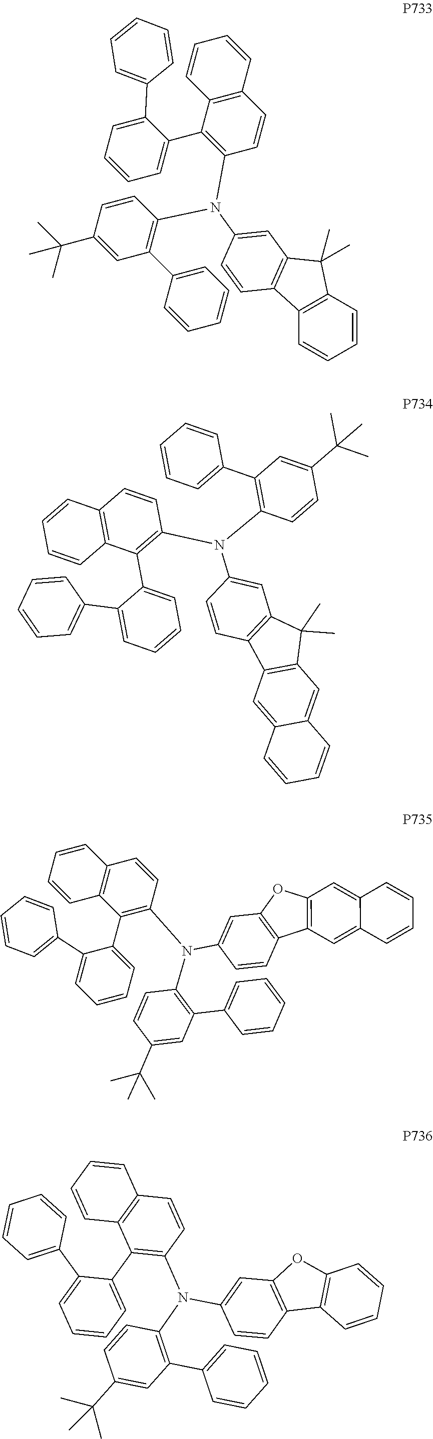 [SMILES: CC(C)(C)c1ccc(N(c2ccc3c(c2)C(C)(C)c2ccccc2-3)c2ccc3ccccc3c2-c2ccccc2-c2ccccc2)c(-c2ccccc2)c1 ; CC(C)(C)c1ccc(N(c2ccc3c(c2)C(C)(C)c2cc4ccccc4cc2-3)c2ccc3ccccc3c2-c2ccccc2-c2ccccc2)c(-c2ccccc2)c1 ; CC(C)(C)c1ccc(N(c2ccc3c(c2)oc2cc4ccccc4cc23)c2ccc3ccccc3c2-c2ccccc2-c2ccccc2)c(-c2ccccc2)c1 ; CC(C)(C)c1ccc(N(c2ccc3c(c2)oc2ccccc23)c2ccc3ccccc3c2-c2ccccc2-c2ccccc2)c(-c2ccccc2)c1]

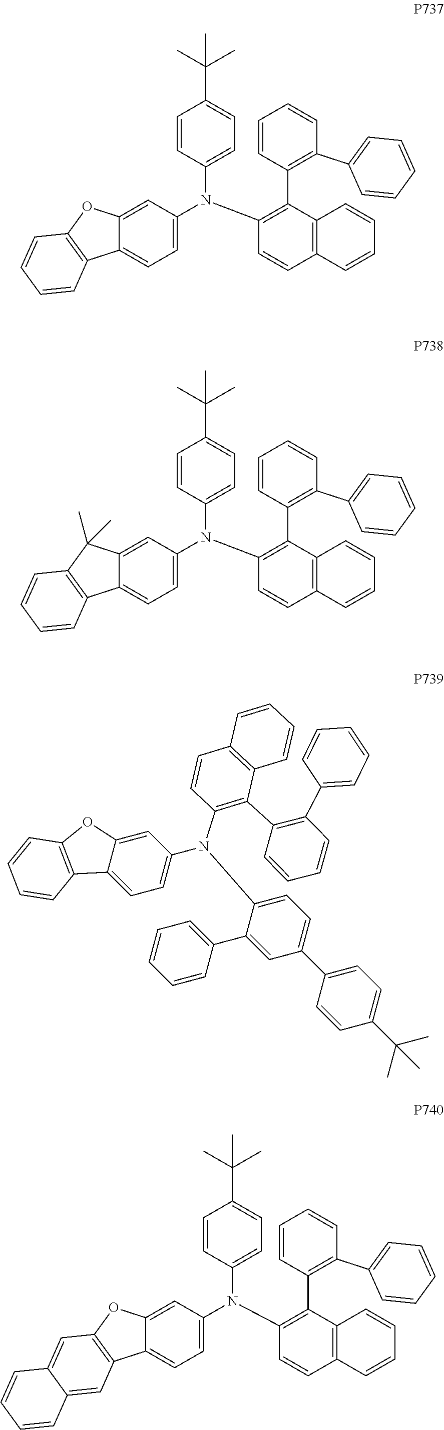 [SMILES: CC(C)(C)c1ccc(N(c2ccc3c(c2)oc2ccccc23)c2ccc3ccccc3c2-c2ccccc2-c2ccccc2)cc1 ; CC(C)(C)c1ccc(N(c2ccc3c(c2)C(C)(C)c2ccccc2-3)c2ccc3ccccc3c2-c2ccccc2-c2ccccc2)cc1 ; CC(C)(C)c1ccc(-c2ccc(N(c3ccc4c(c3)oc3ccccc34)c3ccc4ccccc4c3-c3ccccc3-c3ccccc3)c(-c3ccccc3)c2)cc1 ; CC(C)(C)c1ccc(N(c2ccc3c(c2)oc2cc4ccccc4cc23)c2ccc3ccccc3c2-c2ccccc2-c2ccccc2)cc1]

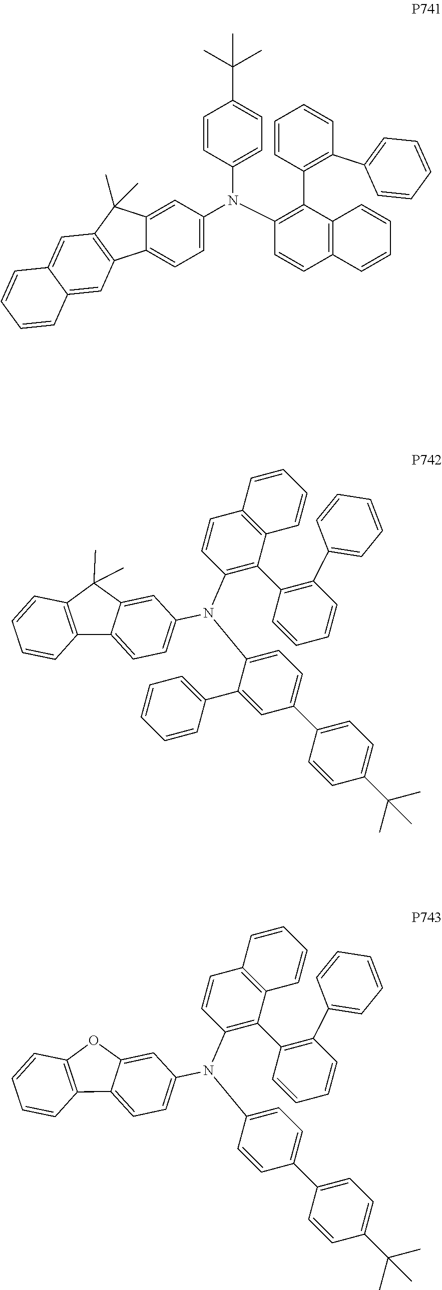 [SMILES: CC(C)(C)c1ccc(N(c2ccc3c(c2)C(C)(C)c2cc4ccccc4cc2-3)c2ccc3ccccc3c2-c2ccccc2-c2ccccc2)cc1 ; CC(C)(C)c1ccc(-c2ccc(N(c3ccc4c(c3)C(C)(C)c3ccccc3-4)c3ccc4ccccc4c3-c3ccccc3-c3ccccc3)c(-c3ccccc3)c2)cc1 ; CC(C)(C)c1ccc(-c2ccc(N(c3ccc4c(c3)oc3ccccc34)c3ccc4ccccc4c3-c3ccccc3-c3ccccc3)cc2)cc1]

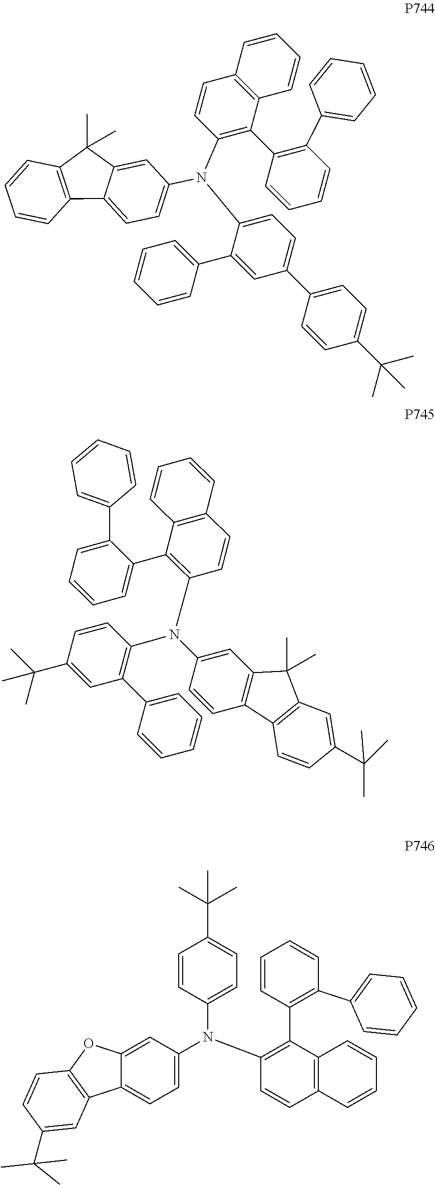

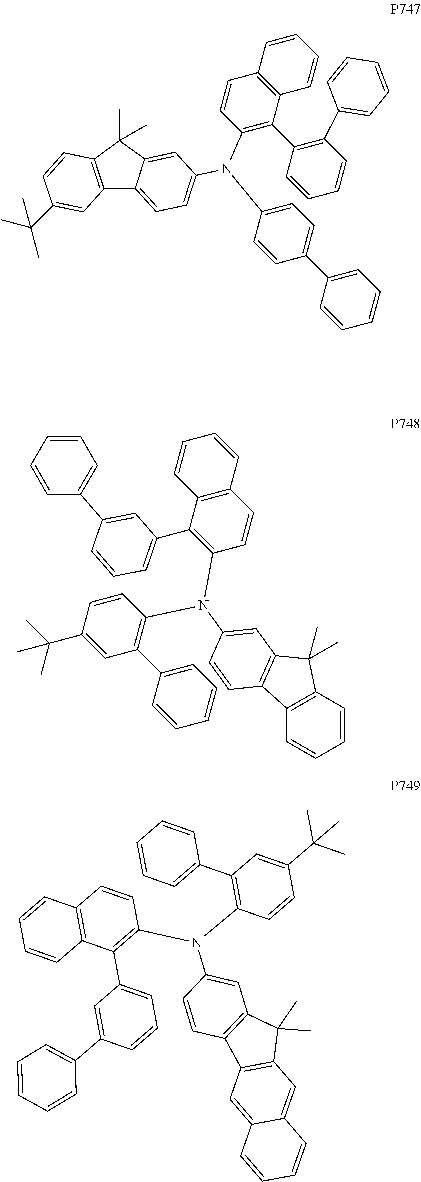 [SMILES: CC(C)(C)c1ccc2c(c1)-c1ccc(N(c3ccc(-c4ccccc4)cc3)c3ccc4ccccc4c3-c3ccccc3-c3ccccc3)cc1C2(C)C ; CC(C)(C)c1ccc(N(c2ccc3c(c2)C(C)(C)c2ccccc2-3)c2ccc3ccccc3c2-c2cccc(-c3ccccc3)c2)c(-c2ccccc2)c1 ; CC(C)(C)c1ccc(N(c2ccc3c(c2)C(C)(C)c2cc4ccccc4cc2-3)c2ccc3ccccc3c2-c2cccc(-c3ccccc3)c2)c(-c2ccccc2)c1]

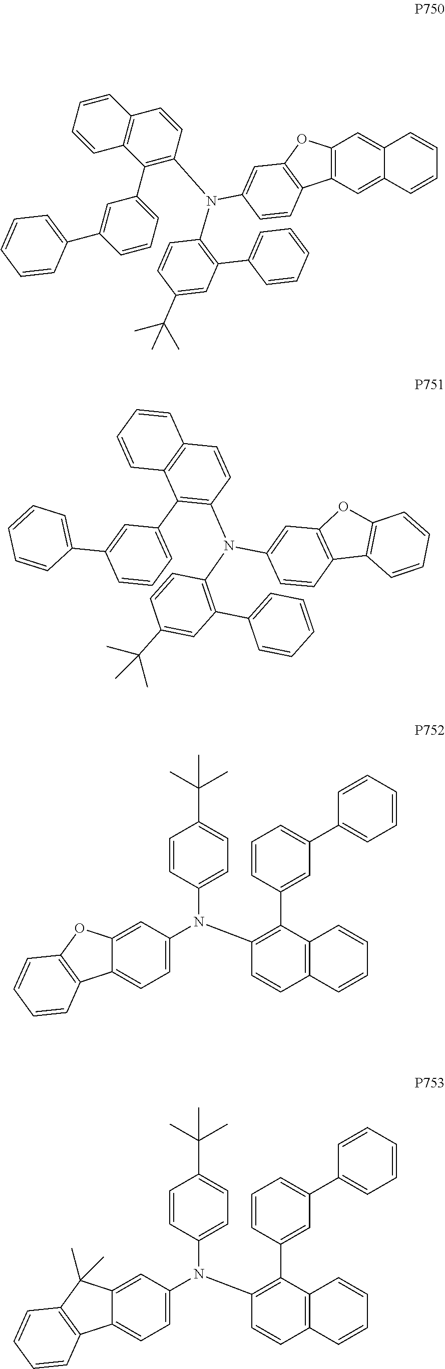

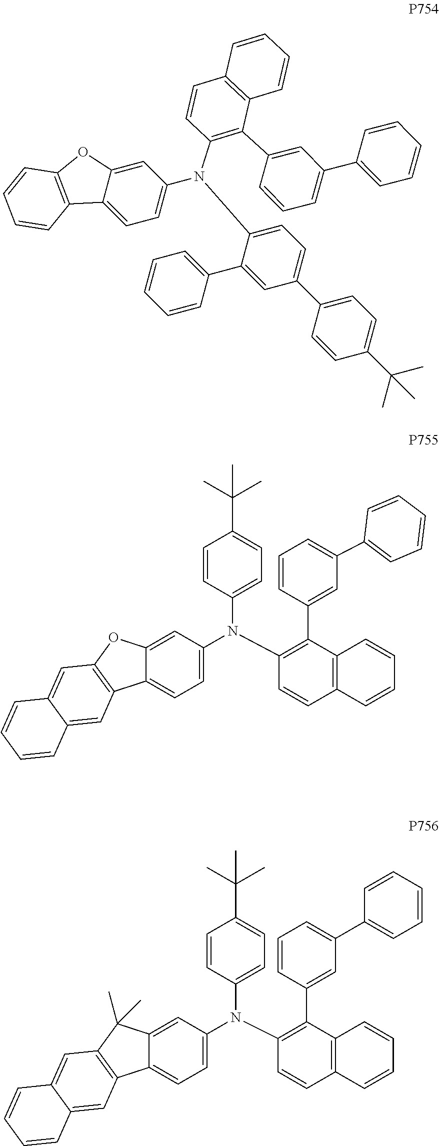 [SMILES: CC(C)(C)c1ccc(-c2ccc(N(c3ccc4c(c3)oc3ccccc34)c3ccc4ccccc4c3-c3cccc(-c4ccccc4)c3)c(-c3ccccc3)c2)cc1 ; CC(C)(C)c1ccc(N(c2ccc3c(c2)oc2cc4ccccc4cc23)c2ccc3ccccc3c2-c2cccc(-c3ccccc3)c2)cc1 ; CC(C)(C)c1ccc(N(c2ccc3c(c2)C(C)(C)c2cc4ccccc4cc2-3)c2ccc3ccccc3c2-c2cccc(-c3ccccc3)c2)cc1]

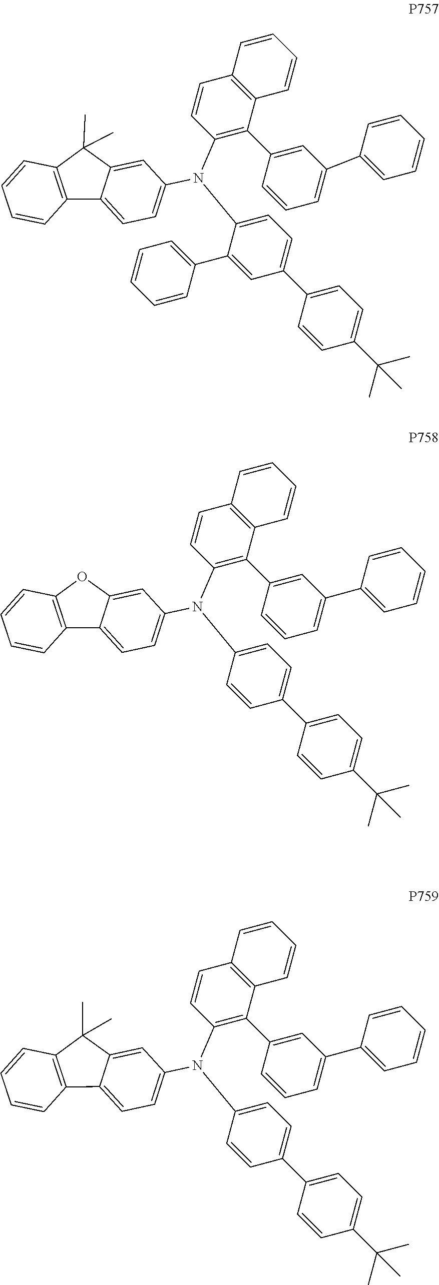 [SMILES: CC(C)(C)c1ccc(-c2ccc(N(c3ccc4c(c3)C(C)(C)c3ccccc3-4)c3ccc4ccccc4c3-c3cccc(-c4ccccc4)c3)c(-c3ccccc3)c2)cc1 ; CC(C)(C)c1ccc(-c2ccc(N(c3ccc4c(c3)oc3ccccc34)c3ccc4ccccc4c3-c3cccc(-c4ccccc4)c3)cc2)cc1 ; CC(C)(C)c1ccc(-c2ccc(N(c3ccc4c(c3)C(C)(C)c3ccccc3-4)c3ccc4ccccc4c3-c3cccc(-c4ccccc4)c3)cc2)cc1]

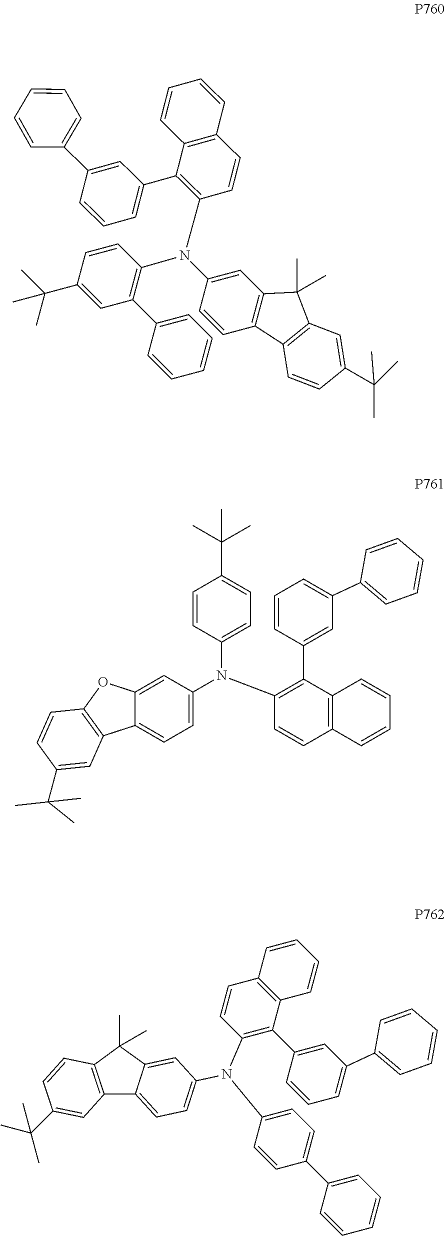

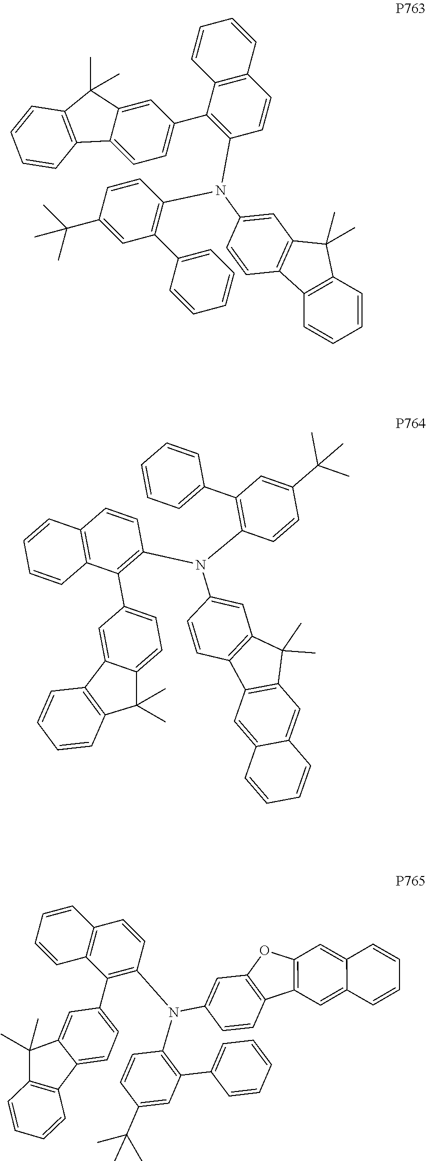

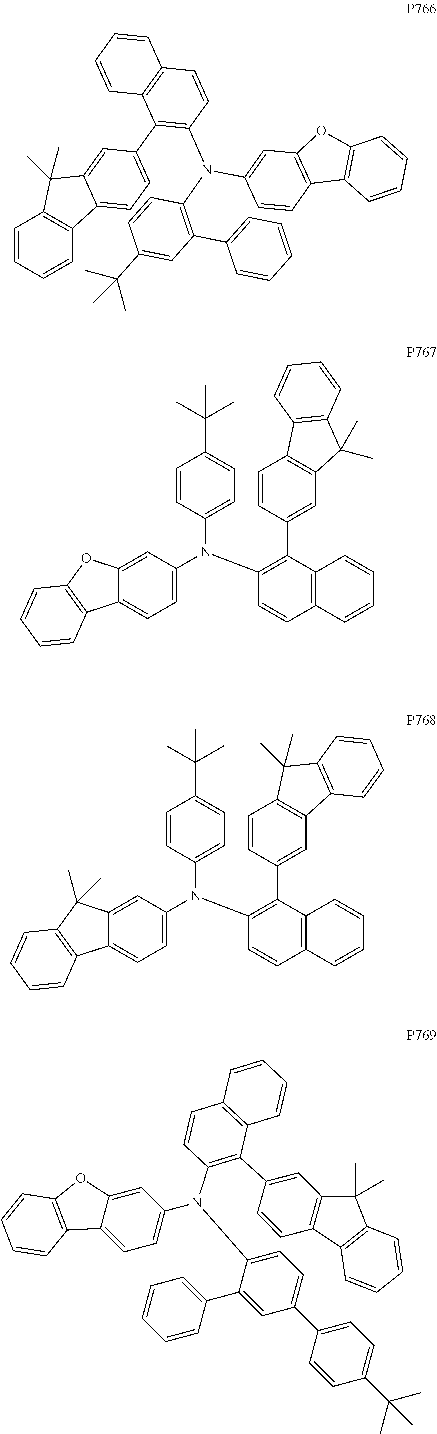 [SMILES: CC(C)(C)c1ccc(N(c2ccc3c(c2)oc2ccccc23)c2ccc3ccccc3c2-c2ccc3c(c2)C(C)(C)c2ccccc2-3)c(-c2ccccc2)c1 ; CC(C)(C)c1ccc(N(c2ccc3c(c2)oc2ccccc23)c2ccc3ccccc3c2-c2ccc3c(c2)C(C)(C)c2ccccc2-3)cc1 ; CC(C)(C)c1ccc(N(c2ccc3c(c2)C(C)(C)c2ccccc2-3)c2ccc3ccccc3c2-c2ccc3c(c2)-c2ccccc2C3(C)C)cc1 ; CC(C)(C)c1ccc(-c2ccc(N(c3ccc4c(c3)oc3ccccc34)c3ccc4ccccc4c3-c3ccc4c(c3)C(C)(C)c3ccccc3-4)c(-c3ccccc3)c2)cc1]

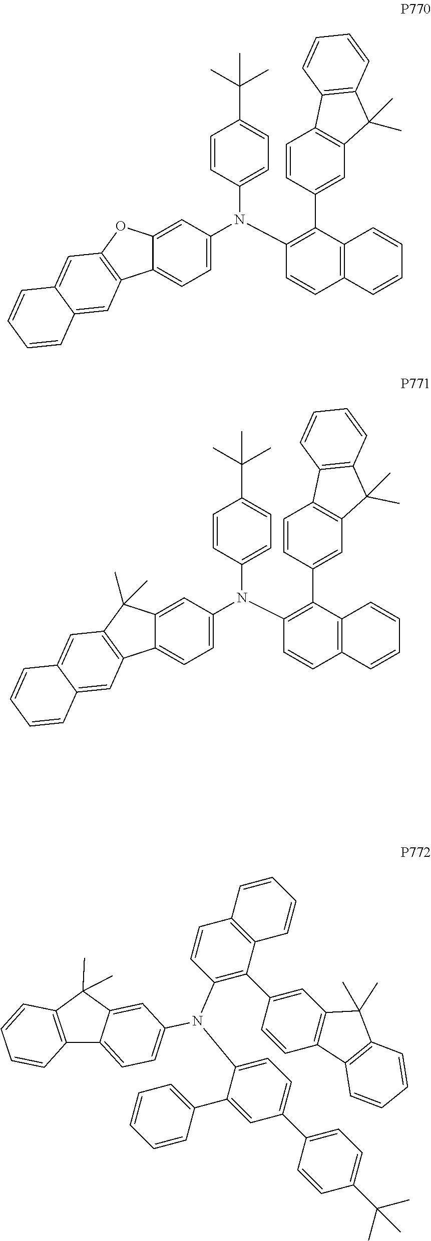 [SMILES: CC(C)(C)c1ccc(N(c2ccc3c(c2)oc2cc4ccccc4cc23)c2ccc3ccccc3c2-c2ccc3c(c2)C(C)(C)c2ccccc2-3)cc1 ; CC(C)(C)c1ccc(N(c2ccc3c(c2)C(C)(C)c2cc4ccccc4cc2-3)c2ccc3ccccc3c2-c2ccc3c(c2)C(C)(C)c2ccccc2-3)cc1 ; CC(C)(C)c1ccc(-c2ccc(N(c3ccc4c(c3)C(C)(C)c3ccccc3-4)c3ccc4ccccc4c3-c3ccc4c(c3)C(C)(C)c3ccccc3-4)c(-c3ccccc3)c2)cc1]

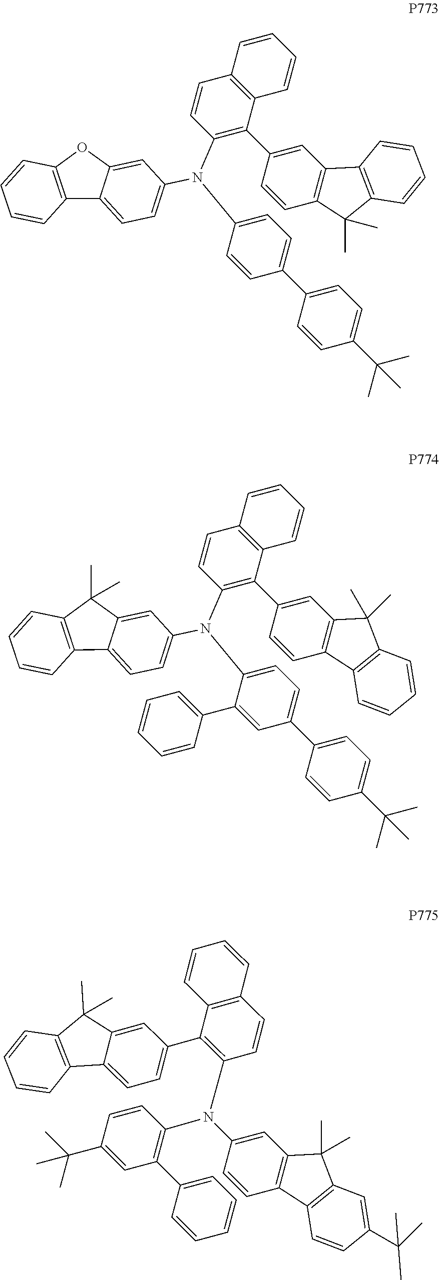 [SMILES: CC(C)(C)c1ccc(-c2ccc(N(c3ccc4c(c3)oc3ccccc34)c3ccc4ccccc4c3-c3ccc4c(c3)-c3ccccc3C4(C)C)cc2)cc1 ; CC(C)(C)c1ccc(-c2ccc(N(c3ccc4c(c3)C(C)(C)c3ccccc3-4)c3ccc4ccccc4c3-c3ccc4c(c3)C(C)(C)c3ccccc3-4)c(-c3ccccc3)c2)cc1 ; CC(C)(C)c1ccc(N(c2ccc3c(c2)C(C)(C)c2cc(C(C)(C)C)ccc2-3)c2ccc3ccccc3c2-c2ccc3c(c2)C(C)(C)c2ccccc2-3)c(-c2ccccc2)c1]

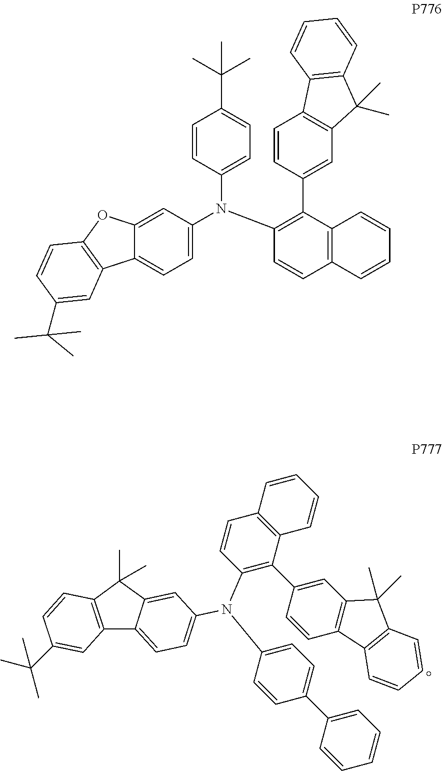 [SMILES: CC(C)(C)c1ccc(N(c2ccc3c(c2)oc2ccc(C(C)(C)C)cc23)c2ccc3ccccc3c2-c2ccc3c(c2)C(C)(C)c2ccccc2-3)cc1 ; CC(C)(C)c1ccc2c(c1)-c1ccc(N(c3ccc(-c4ccccc4)cc3)c3ccc4ccccc4c3-c3ccc4c(c3)C(C)(C)c3ccccc3-4)cc1C2(C)C]

The second object of the disclosure is to provide a use of the compound as described in the first object, and the compound is used in an organic electroluminescent device.

Preferably, the compound is used as an electron blocking layer material in the organic electroluminescent device.

The third object of the disclosure is to provide an organic electroluminescent device, which includes a first electrode, a second electrode and at least one organic layer inserted between the first electrode and the second electrode, and the organic layer contains at least one compound as described in the first object.

When the brightness of the organic electroluminescent device containing the compound of formula I provided by the disclosure reaches 3000 cd/m², the driving voltage is as low as 3.8 V and below, and the current efficiency is as high as 18.2 cd/A and above.

Preferably, the organic layer includes an electron blocking layer, and the electron blocking layer contains at least one compound as described in the first object.

The compound of the disclosure can be applied not only to organic electroluminescent devices, but also to other types of organic electronic devices, including organic field-effect transistors, organic thin film solar cells, information labels, electronic artificial skin sheets, sheet type scanners or electronic papers.

In particular, another technical solution of the disclosure provides an organic electroluminescent device, including a substrate, an anode layer, a plurality of light-emitting functional layers and a cathode layer formed on the substrate in sequence; the light-emitting functional layer includes at least one of a hole injection layer, a hole transporting layer, a light emitting layer, an electron blocking layer, and an electron transporting layer, wherein the electron blocking layer contains at least one of the aforementioned compounds.

The OLED includes an organic material layer at the first electrode and the second electrode as well as between the electrodes. The organic material layer can be divided into multiple regions. For example, the organic material layer may include a hole transporting region, a light emitting layer, and an electron transporting region.

In specific embodiments, a substrate can be used below the first electrode or above the second electrode. The substrate is glass or a polymer material with excellent mechanical strength, thermal stability, water resistance and transparency. In addition, the substrate used as the display can also be equipped with thin film transistors (TFTs).

The first electrode can be formed by sputtering or depositing a material used as the first electrode on the substrate. When the first electrode is used as the anode, an oxide transparent conductive material such as indium tin oxide (ITO), indium zinc oxide (IZO), tin dioxide (SnO₂), zinc oxide (ZnO), etc, and any combination thereof can be used. When the first electrode is used as the cathode, a metal or alloy such as magnesium (Mg), silver (Ag), aluminum (Al), aluminum-lithium (Al—Li), calcium (Ca), magnesium-indium (Mg—In), magnesium-silver (Mg—Ag), etc., as well as any combination thereof can be used.

The organic material layer can be formed on the electrode by methods such as vacuum thermal evaporation, rotary coating, and printing, etc. The compounds used as the organic material layer can be an organic small molecule, an organic macromolecule and a polymer, and a combination thereof.

The hole transporting region is located between the anode and the light emitting layer. The hole transporting region can be a hole transporting layer (HTL) with a single-layer structure, including a single-layer hole transporting layer containing only one compound and a single-layer hole transporting layer containing multiple compounds. The hole transporting region can also be a multilayer structure including at least one of a hole injection layer (HIL), a hole transporting layer (HTL) and an electron blocking layer (EBL). The electron blocking layer uses the compound as shown in Formula I of the disclosure.

The materials in the hole transporting region can be selected from the group consisting of, but not limited to a phthalocyanine derivative such as CuPc, a conductive polymer or a polymer containing a conductive dopant such as polyphenylenevinylene, polyaniline/dodecyl benzene sulfonic acid (Pani/DBSA), poly(3,4-ethylenedioxythiophene)/poly(4-styrenesulfonate) (PEDOT/PSS), polyaniline/camphor sulfonic acid (Pani/CSA), polyaniline/poly(4-styrenesulfonate) (Pani/PSS), an aromatic amine derivative, wherein the aromatic amine derivative comprises compounds as shown in HIT-1 to HT-34 below; or any combination thereof.

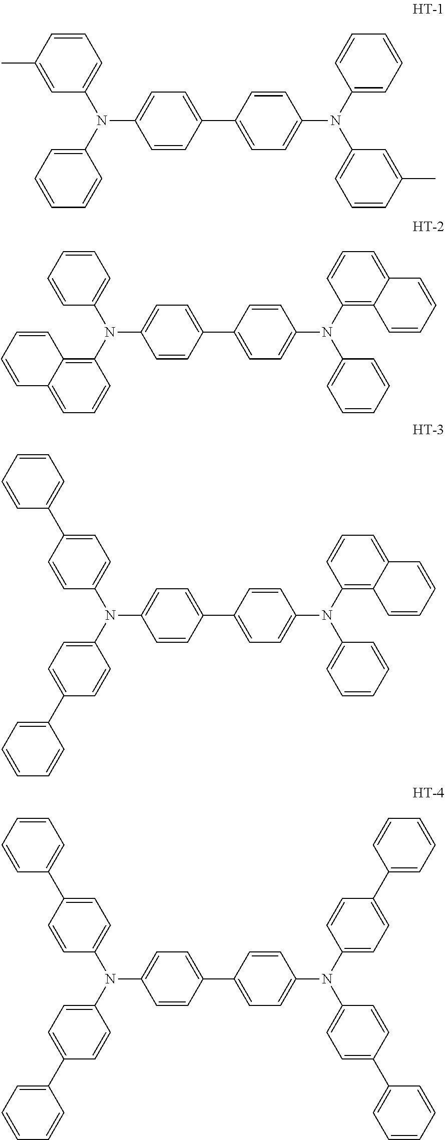

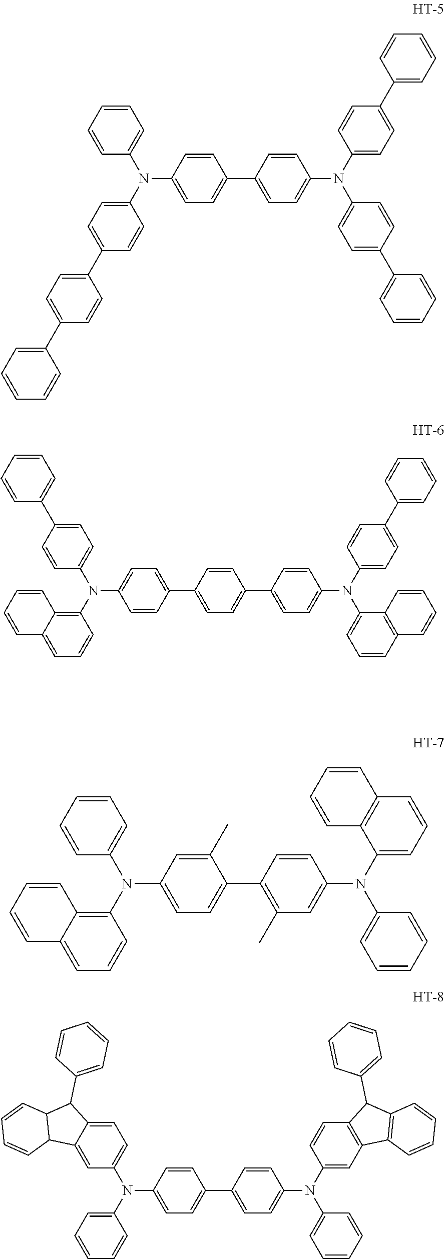 [SMILES: c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(-c2ccc(N(c3ccc(-c4ccc(-c5ccc(N(c6ccc(-c7ccccc7)cc6)c6cccc7ccccc67)cc5)cc4)cc3)c3cccc4ccccc34)cc2)cc1 ; Cc1cc(N(c2ccccc2)c2cccc3ccccc23)ccc1-c1ccc(N(c2ccccc2)c2cccc3ccccc23)cc1C ; C1=CC2c3cc(N(c4ccccc4)c4ccc(-c5ccc(N(c6ccccc6)c6ccc7c(c6)-c6ccccc6C7c6ccccc6)cc5)cc4)ccc3C(c3ccccc3)C2C=C1]

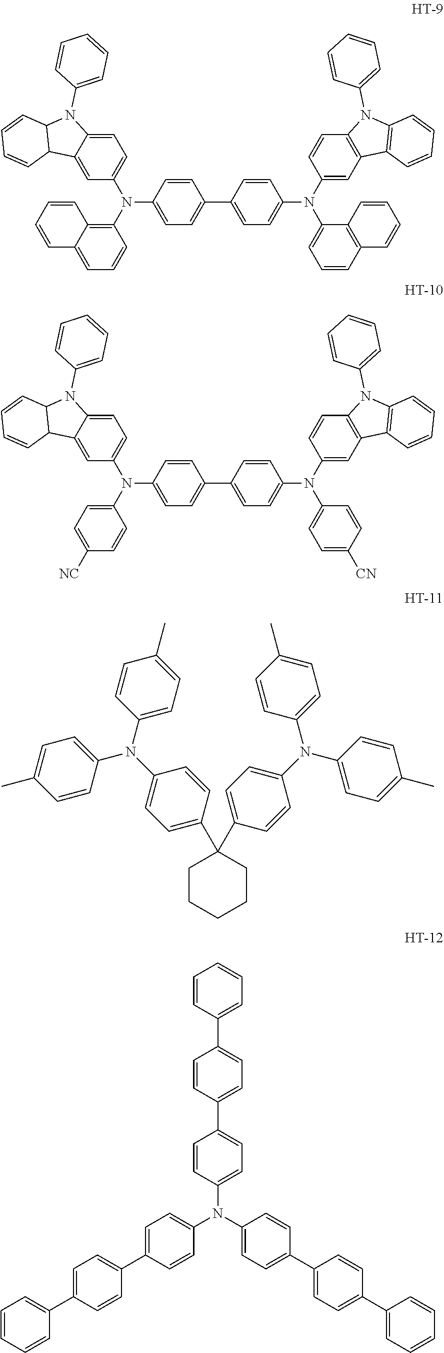

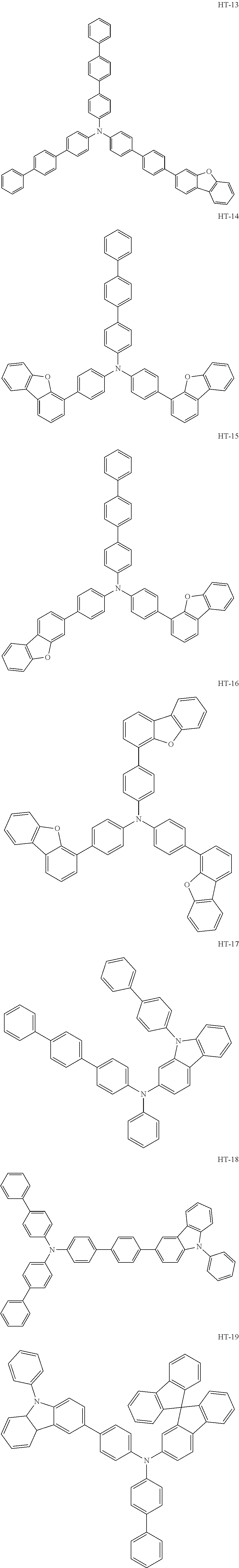

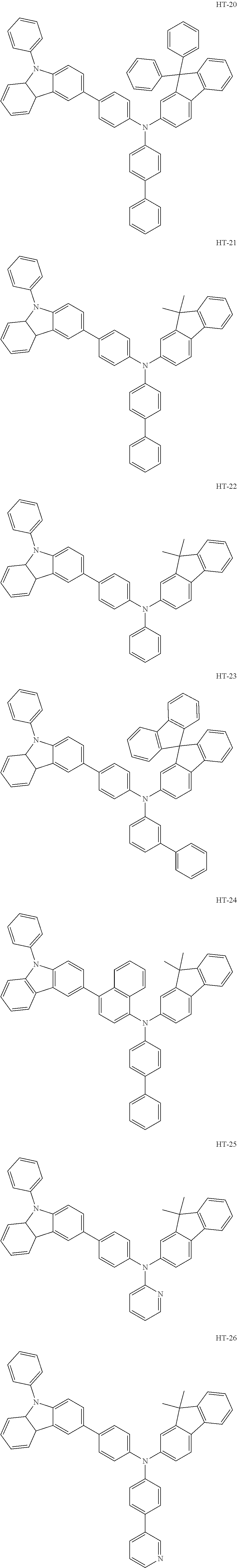

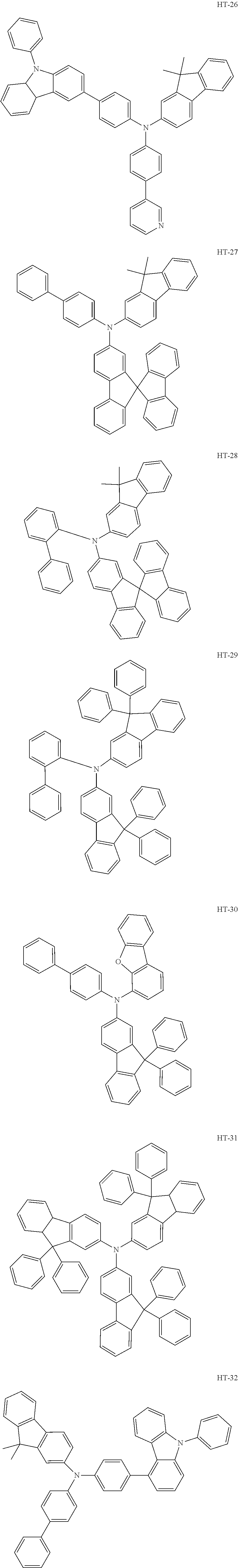

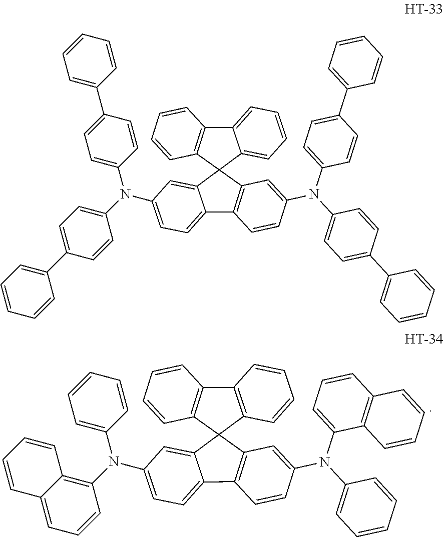 [SMILES: c1ccc(-c2ccc(N(c3ccc(-c4ccccc4)cc3)c3ccc4c(c3)C3(c5ccccc5-c5ccccc53)c3cc(N(c5ccc(-c6ccccc6)cc5)c5ccc(-c6ccccc6)cc5)ccc3-4)cc2)cc1 ; c1ccc(N(c2ccc3c(c2)C2(c4ccccc4-c4ccccc42)c2cc(N(c4ccccc4)c4cccc5ccccc45)ccc2-3)c2cccc3ccccc23)cc1]

The hole injection layer is located between the anode and the hole transporting layer. The hole injection layer can be a single compound material or a combination of multiple compounds. For example, the hole injection layer can adopt one or more compounds of the above mentioned HT-1 to HT-34, or one or more compounds of the following HI-1 to HI-3; or one or more compounds of HT-1 to HT-34 doped with one or more compounds of the following HI-1 to HI-3.

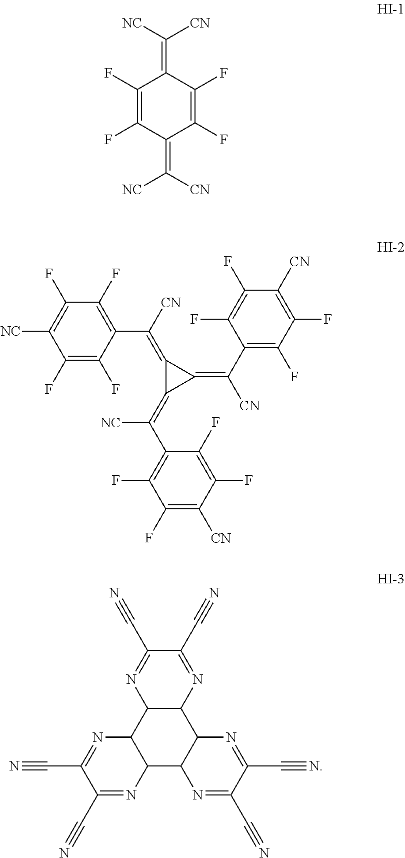 [SMILES: N#CC(C#N)=c1c(F)c(F)c(=C(C#N)C#N)c(F)c1F ; N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; N#CC1=NC2C3N=C(C#N)C(C#N)=NC3C3N=C(C#N)C(C#N)=NC3C2N=C1C#N]

The light emitting layer includes a light emitting dye (i.e. a dopant) that can emit spectra of different wavelengths, and can also include a host material (Host). The light emitting layer may be a monochromatic light emitting layer emitting a single color such as red, green, and blue, etc. The monochromatic light emitting layers with different colors can be arranged in a plane according to the pixel graphics, or stacked together to forma color light emitting layer. When the light emitting layers of different colors are stacked together, they can be separated from each other or connected to each other. The light emitting layer can also be a single color light emitting layer that can emit different colors such as red, green, and blue, etc., at the same time.

According to different technologies, the light emitting layer material can be a fluorescent electroluminescent material, a phosphorescent electroluminescent material, a thermally activated delayed fluorescent light emitting material and other various materials. In an OLED device, a single light emitting technology or a combination of different light emitting technologies can be used. These different light emitting materials classified by technologies can emit light of the same color or light of different colors.

In one aspect of the disclosure, the light emitting layer adopts the technology of fluorescent electroluminescence. The fluorescent host material of the light emitting layer thereof can be selected from the group consisting of, but not limited to one or a combination of more of BFH-1 to BFH-17 as listed below.

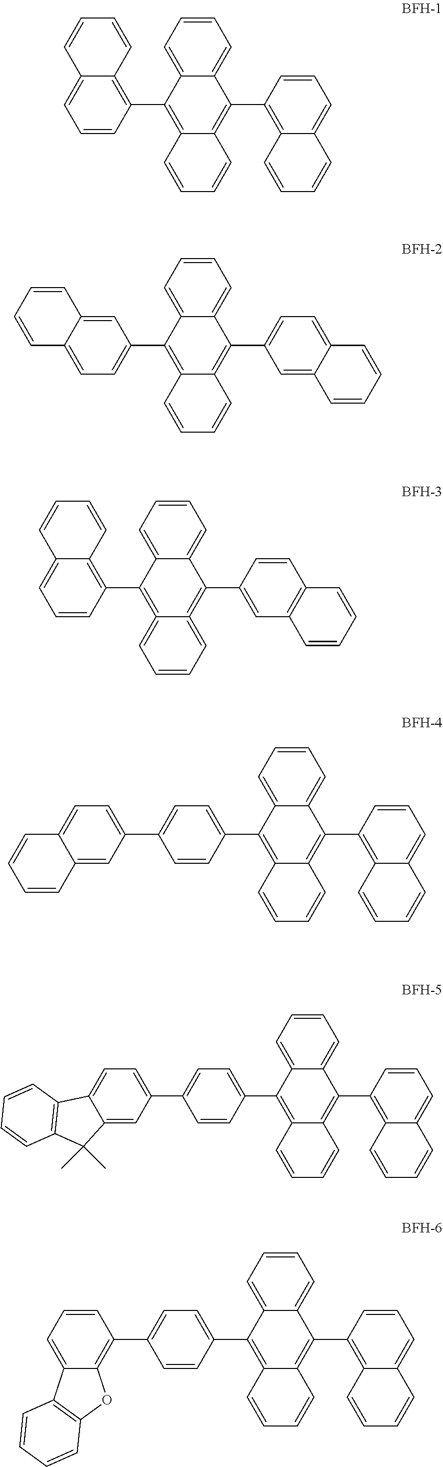

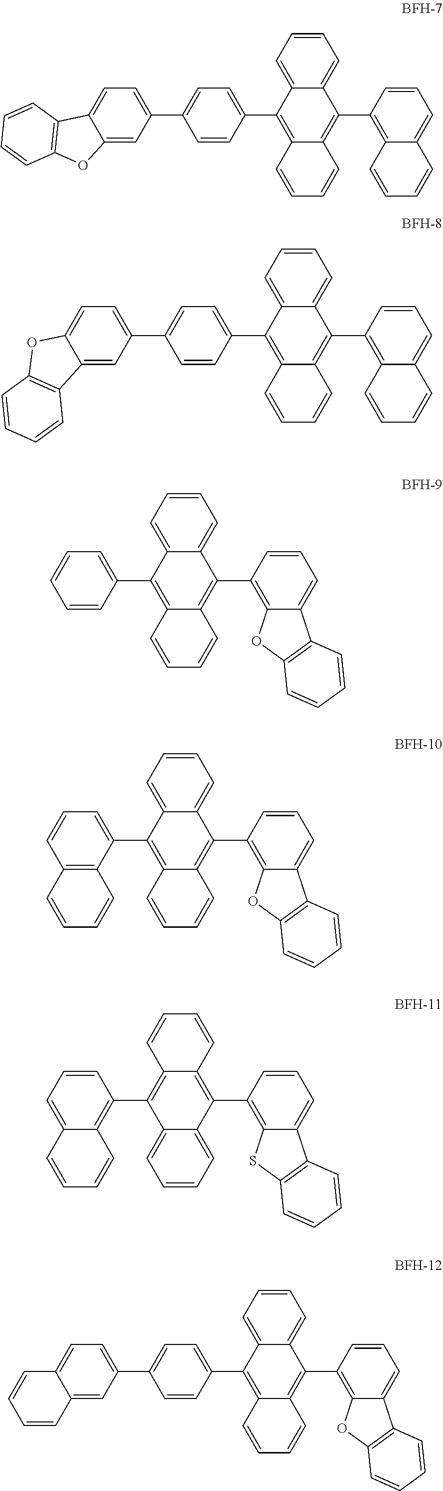 [SMILES: c1ccc2c(-c3c4ccccc4c(-c4ccc(-c5ccc6c(c5)oc5ccccc56)cc4)c4ccccc34)cccc2c1 ; c1ccc2c(-c3c4ccccc4c(-c4ccc(-c5ccc6oc7ccccc7c6c5)cc4)c4ccccc34)cccc2c1 ; c1ccc(-c2c3ccccc3c(-c3cccc4c3oc3ccccc34)c3ccccc23)cc1 ; c1ccc2c(-c3c4ccccc4c(-c4cccc5c4oc4ccccc45)c4ccccc34)cccc2c1 ; c1ccc2c(-c3c4ccccc4c(-c4cccc5c4sc4ccccc45)c4ccccc34)cccc2c1 ; c1ccc2cc(-c3ccc(-c4c5ccccc5c(-c5cccc6c5oc5ccccc56)c5ccccc45)cc3)ccc2c1]

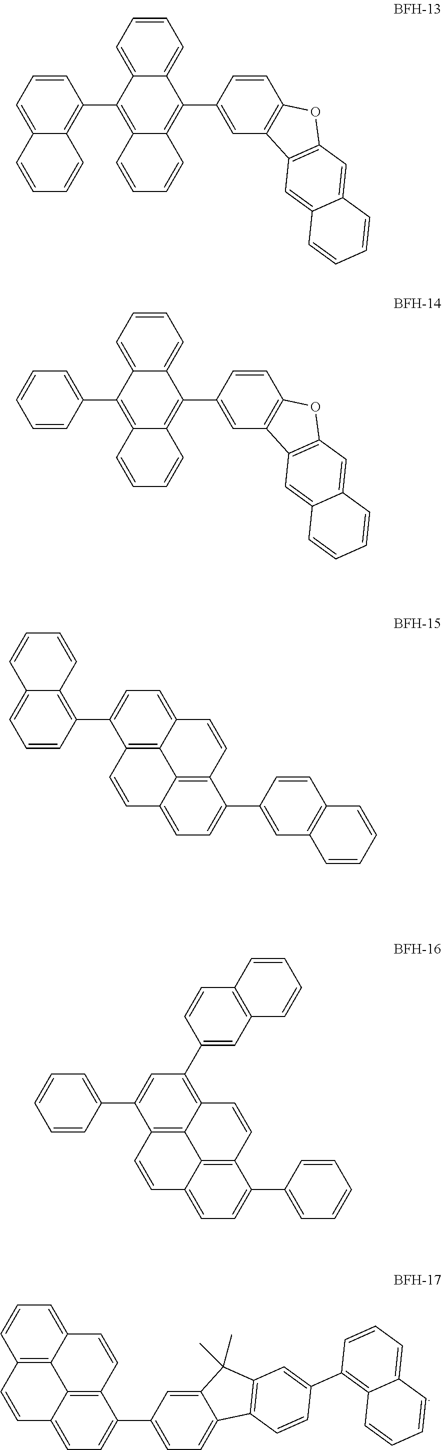

In one aspect of the disclosure, the fluorescent electroluminescence technology is adopted for the light emitting layer. The fluorescent dopant of the light emitting layer thereof can be selected from the group consisting of, but not limited to one or a combination of more of BFD-1 to BFD-12 as listed below.

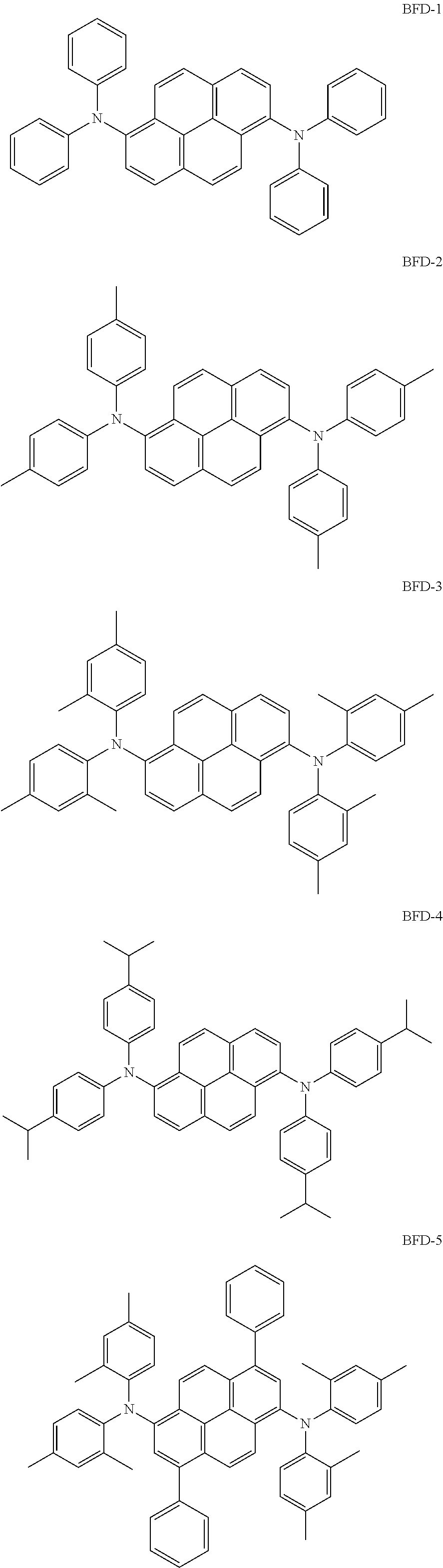

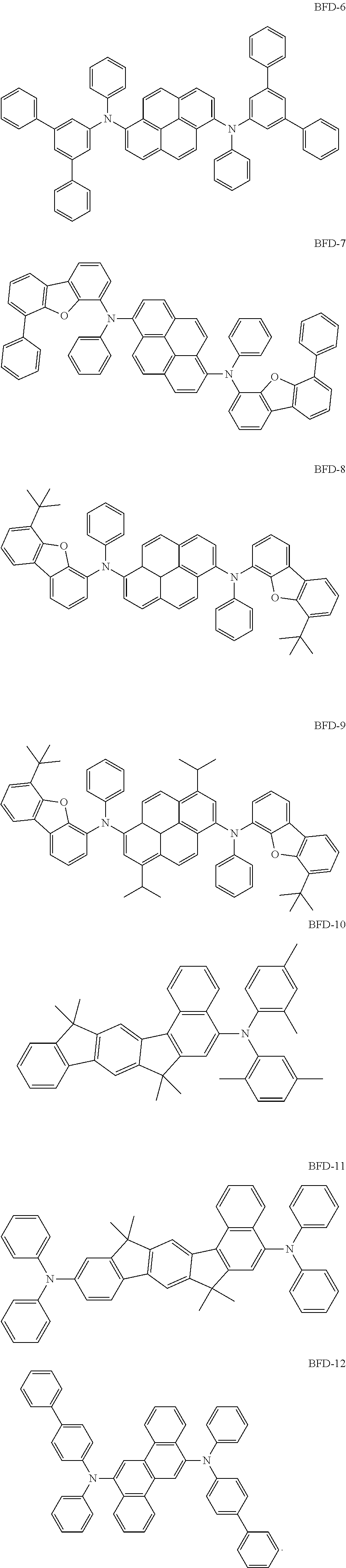

In one aspect of the disclosure, the phosphorescent electroluminescence technology is adopted for the light emitting layer. The host material of the light emitting layer thereof is selected from the group consisting of, but not limited to one or a combination of more of GPH-1 to GPH-80.

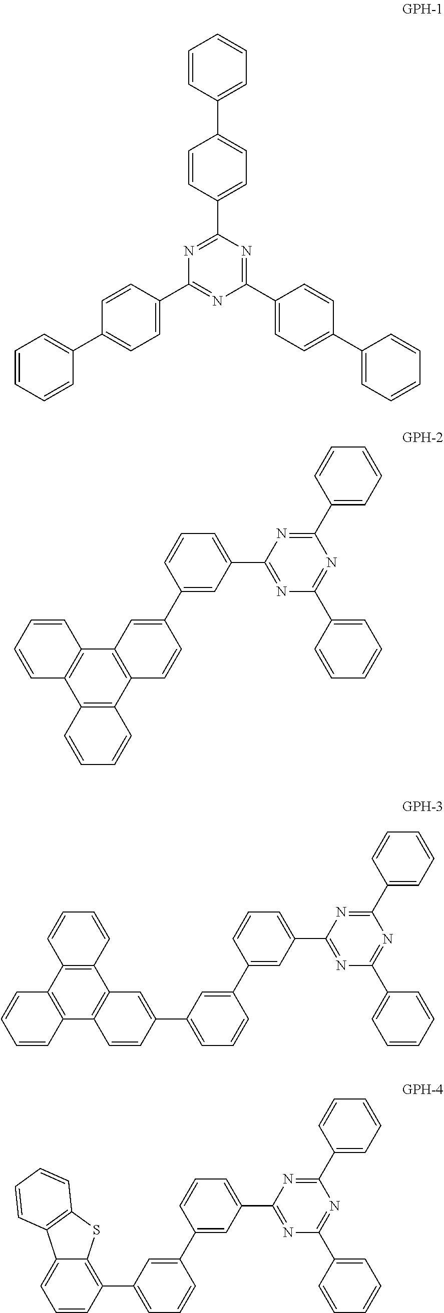 [SMILES: c1ccc(-c2ccc(-c3nc(-c4ccc(-c5ccccc5)cc4)nc(-c4ccc(-c5ccccc5)cc4)n3)cc2)cc1 ; c1ccc(-c2nc(-c3ccccc3)nc(-c3cccc(-c4ccc5c6ccccc6c6ccccc6c5c4)c3)n2)cc1 ; c1ccc(-c2nc(-c3ccccc3)nc(-c3cccc(-c4cccc(-c5ccc6c7ccccc7c7ccccc7c6c5)c4)c3)n2)cc1 ; c1ccc(-c2nc(-c3ccccc3)nc(-c3cccc(-c4cccc(-c5cccc6c5sc5ccccc56)c4)c3)n2)cc1]

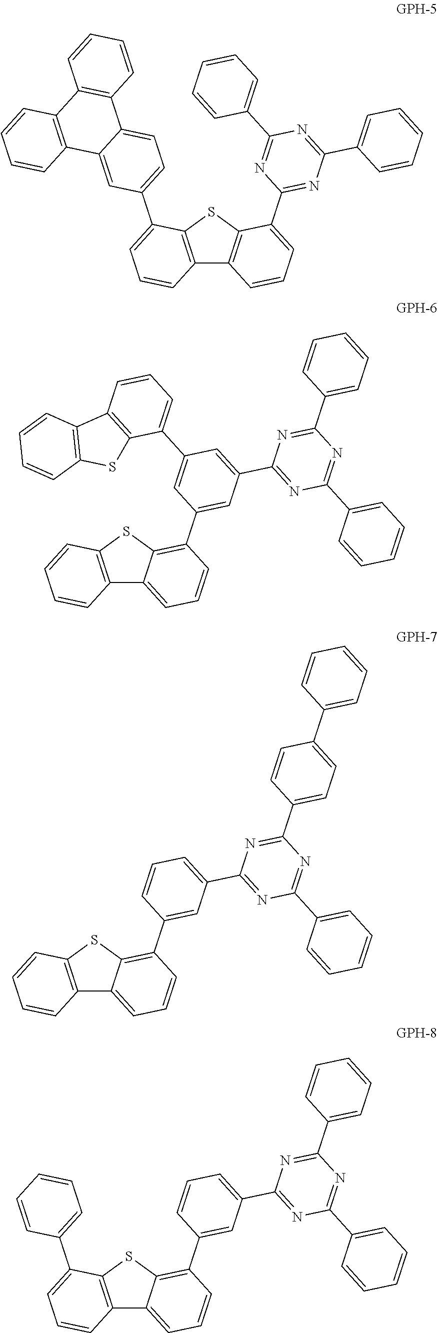 [SMILES: c1ccc(-c2nc(-c3ccccc3)nc(-c3cccc4c3sc3c(-c5ccc6c7ccccc7c7ccccc7c6c5)cccc34)n2)cc1 ; c1ccc(-c2nc(-c3ccccc3)nc(-c3cc(-c4cccc5c4sc4ccccc45)cc(-c4cccc5c4sc4ccccc45)c3)n2)cc1 ; c1ccc(-c2ccc(-c3nc(-c4ccccc4)nc(-c4cccc(-c5cccc6c5sc5ccccc56)c4)n3)cc2)cc1 ; c1ccc(-c2nc(-c3ccccc3)nc(-c3cccc(-c4cccc5c4sc4c(-c6ccccc6)cccc45)c3)n2)cc1]

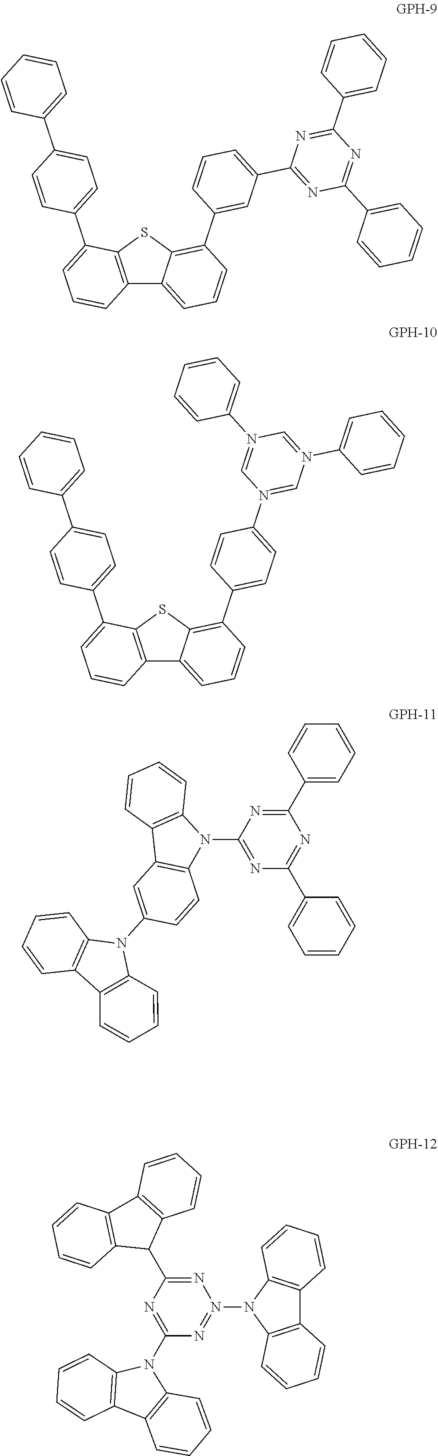

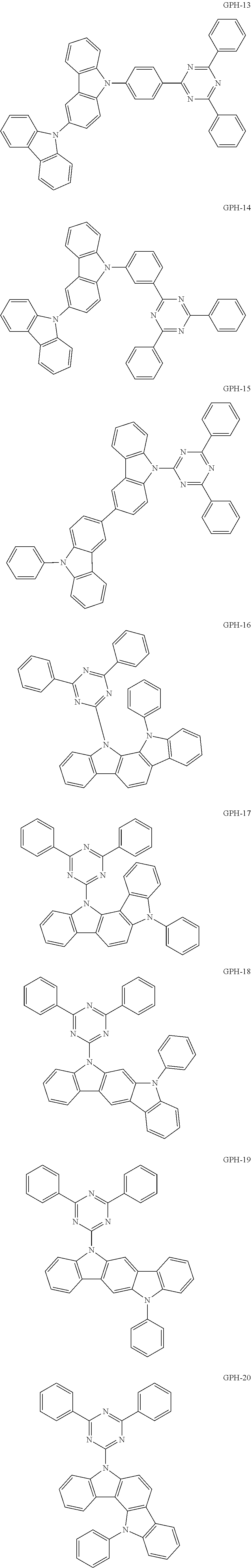

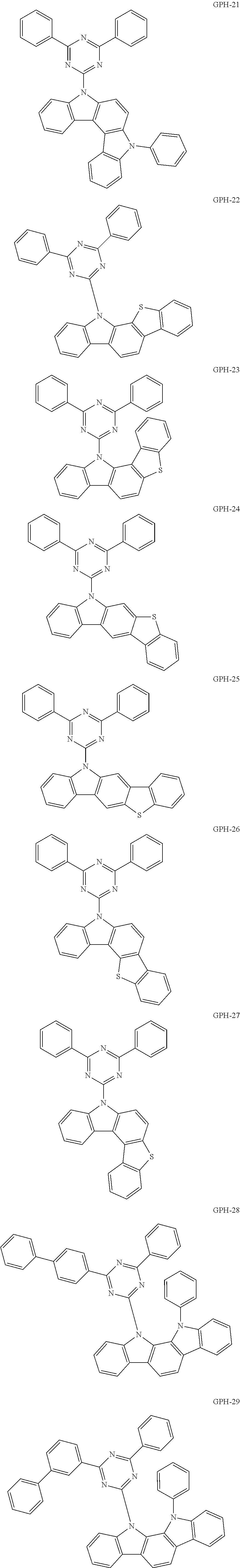

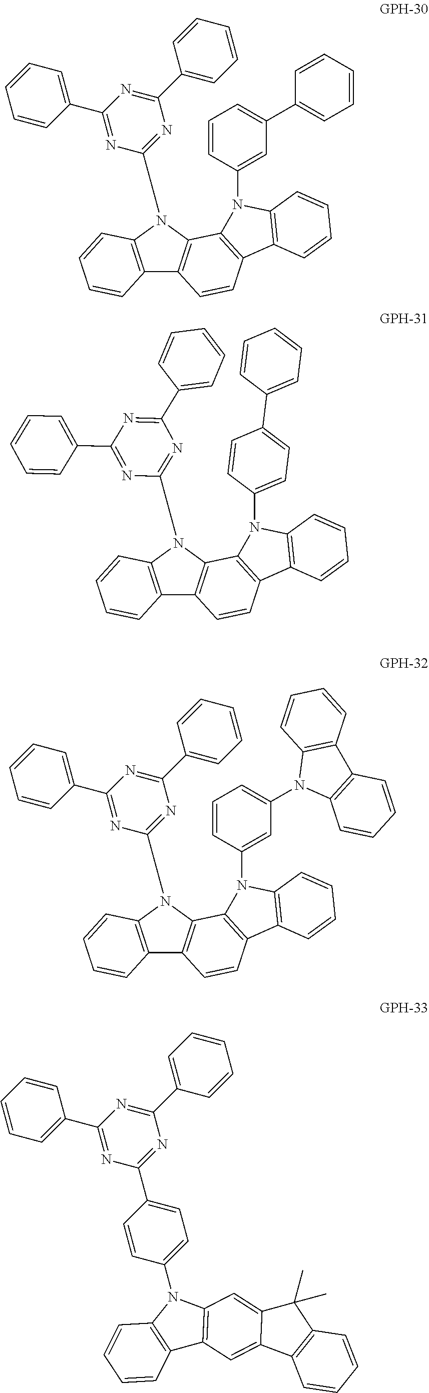

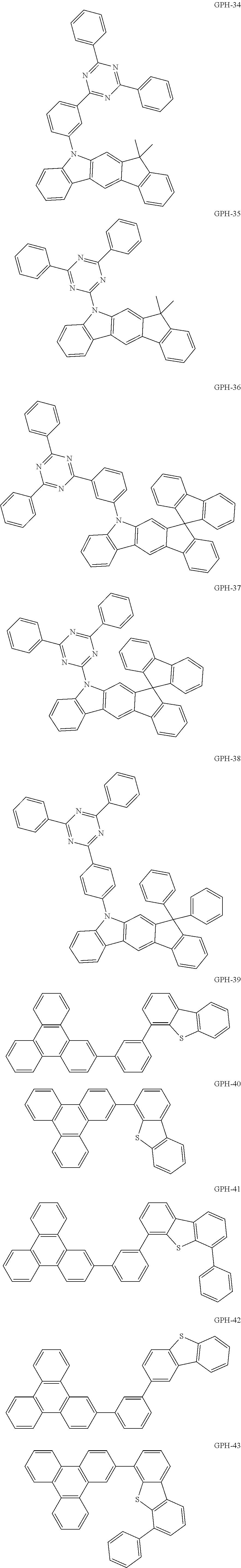

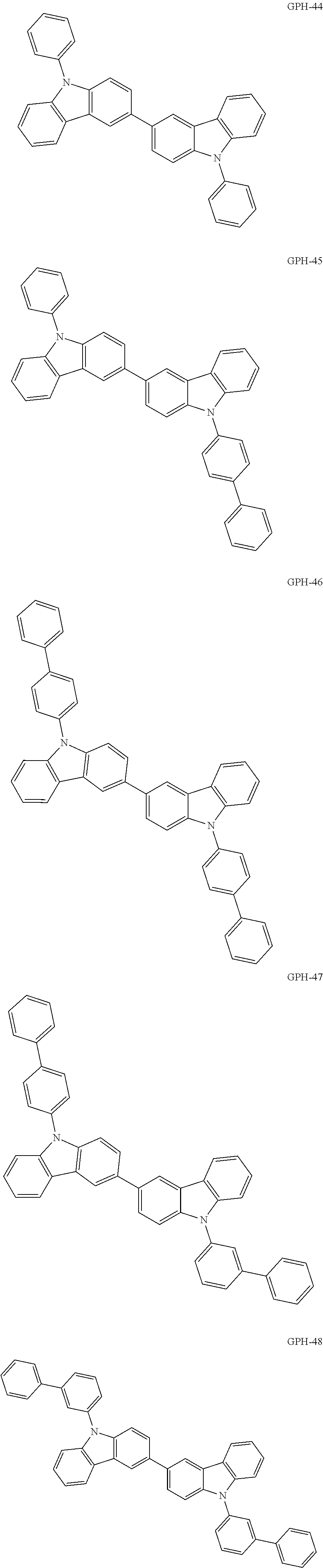 [SMILES: c1ccc(-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccccc5)ccc43)cc2)cc1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1]

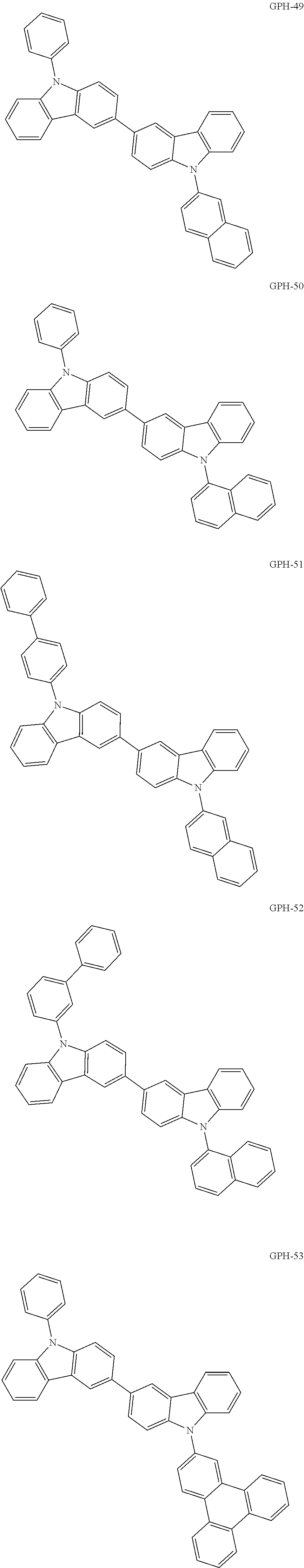 [SMILES: c1ccc(-n2c3ccccc3c3cc(-c4ccc5c(c4)c4ccccc4n5-c4ccc5ccccc5c4)ccc32)cc1 ; c1ccc(-n2c3ccccc3c3cc(-c4ccc5c(c4)c4ccccc4n5-c4cccc5ccccc45)ccc32)cc1 ; c1ccc(-c2ccc(-n3c4ccccc4c4cc(-c5ccc6c(c5)c5ccccc5n6-c5ccc6ccccc6c5)ccc43)cc2)cc1 ; c1ccc(-c2cccc(-n3c4ccccc4c4cc(-c5ccc6c(c5)c5ccccc5n6-c5cccc6ccccc56)ccc43)c2)cc1 ; c1ccc(-n2c3ccccc3c3cc(-c4ccc5c(c4)c4ccccc4n5-c4ccc5c6ccccc6c6ccccc6c5c4)ccc32)cc1]

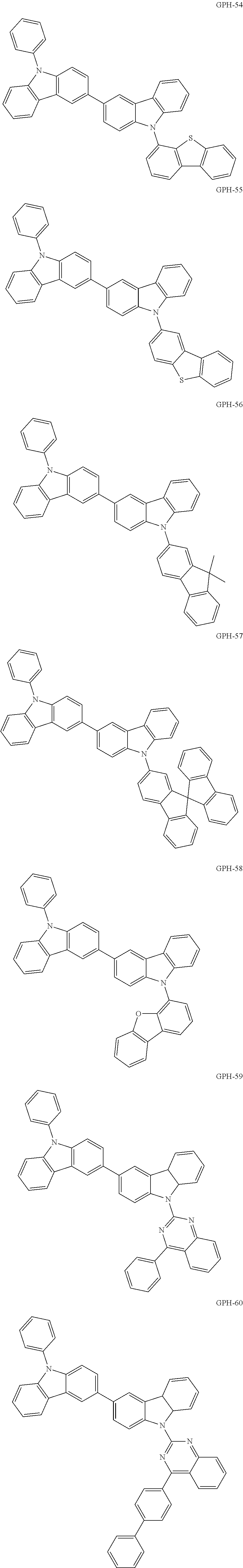

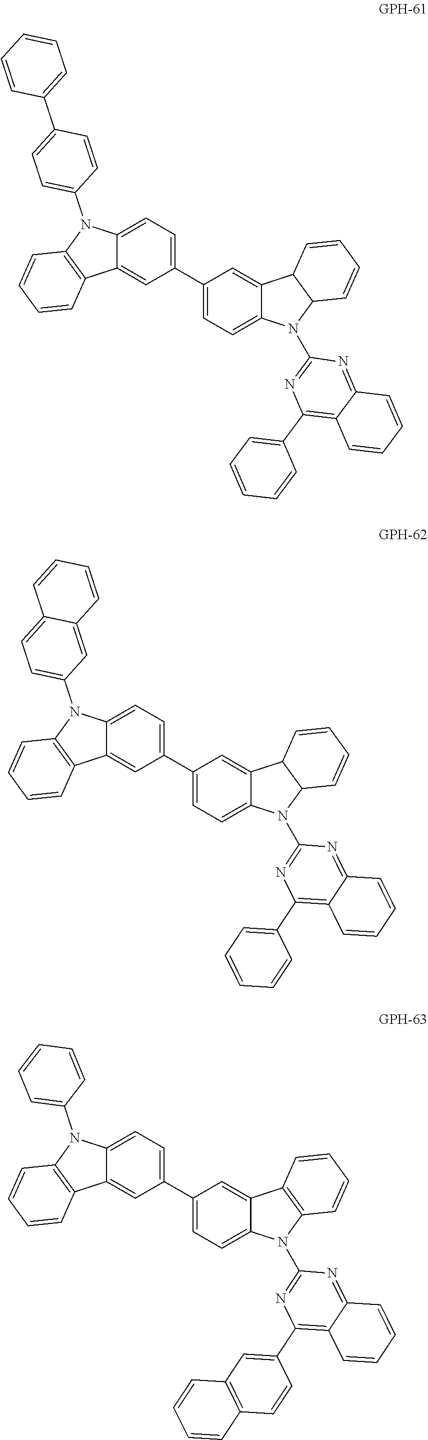 [SMILES: C1=CC2c3cc(-c4ccc5c(c4)c4ccccc4n5-c4ccc(-c5ccccc5)cc4)ccc3N(c3nc(-c4ccccc4)c4ccccc4n3)C2C=C1 ; C1=CC2c3cc(-c4ccc5c(c4)c4ccccc4n5-c4ccc5ccccc5c4)ccc3N(c3nc(-c4ccccc4)c4ccccc4n3)C2C=C1 ; c1ccc(-n2c3ccccc3c3cc(-c4ccc5c(c4)c4ccccc4n5-c4nc(-c5ccc6ccccc6c5)c5ccccc5n4)ccc32)cc1]

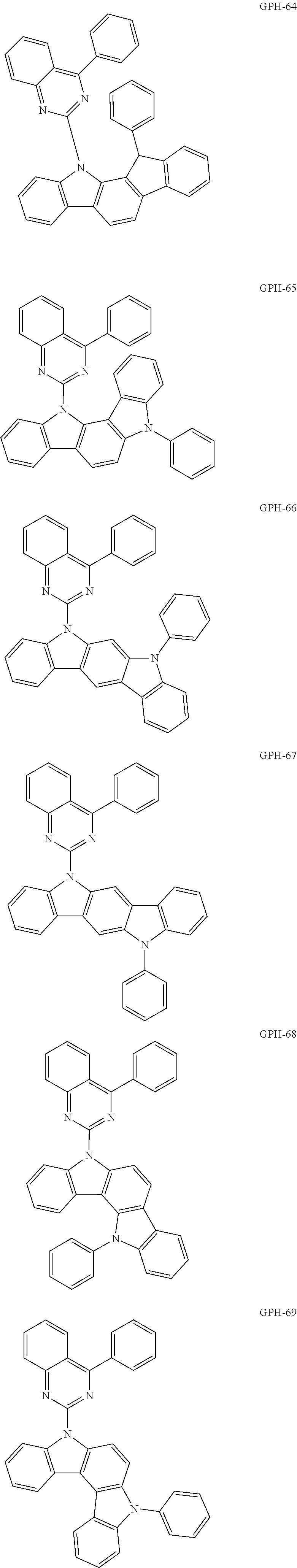

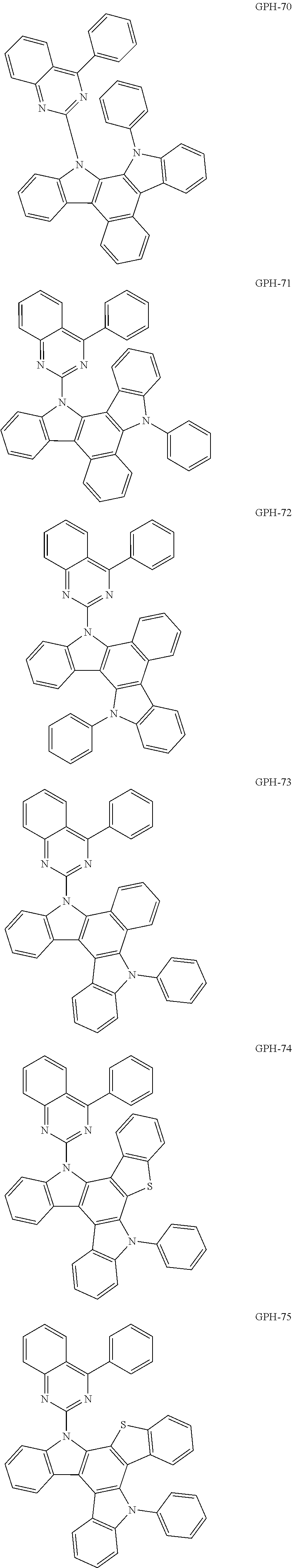

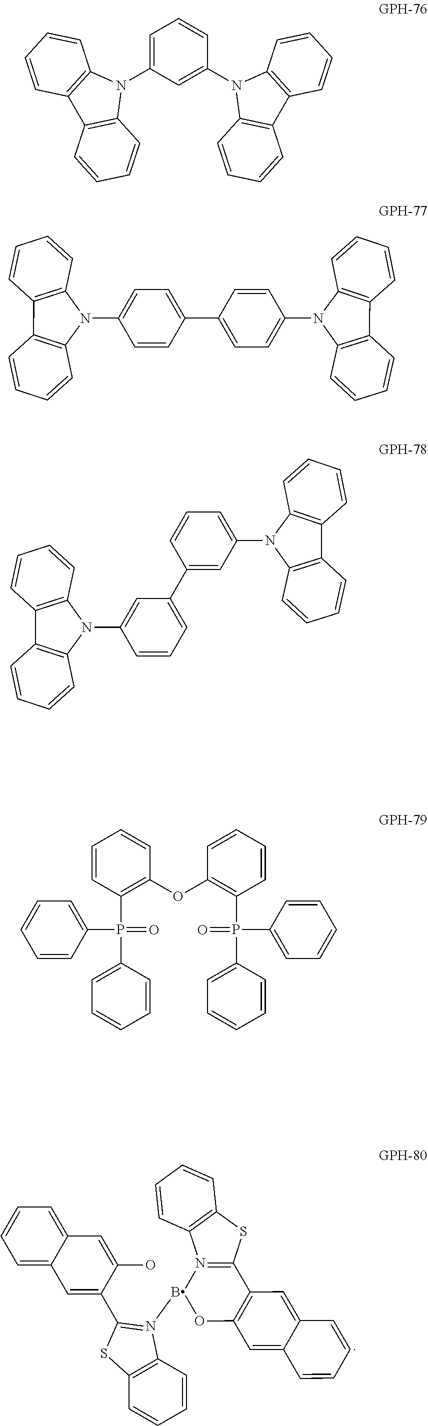

In one aspect of the disclosure, the phosphorescent electroluminescence technology is adopted for the light emitting layer. The phosphorescent dopant of the light emitting layer thereof can be selected from the group consisting of, but not limited to one or a combination of more of GPD-1 to GPD-47 as listed below.

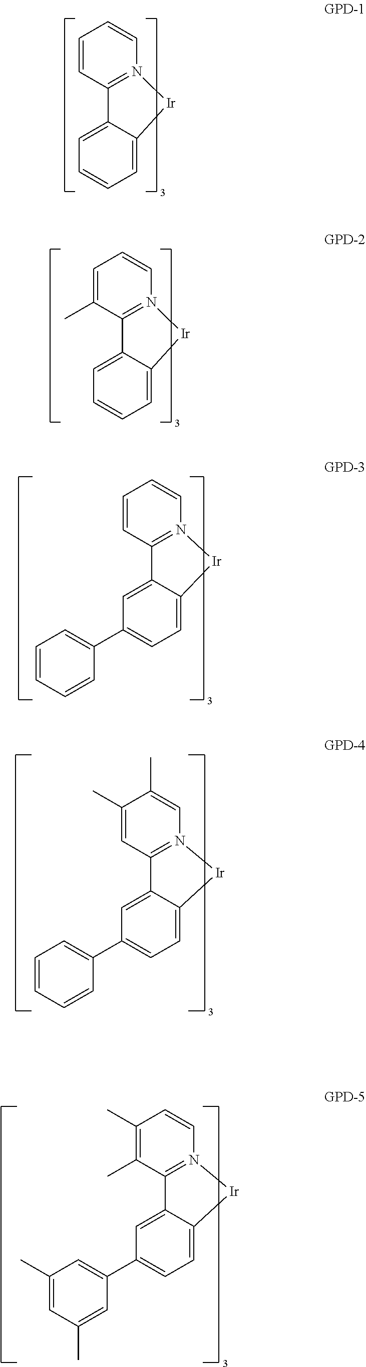

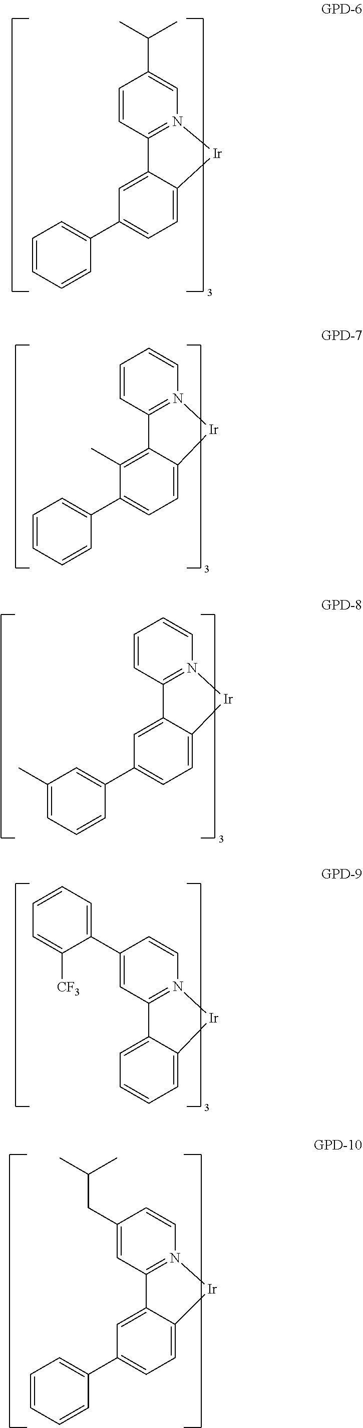

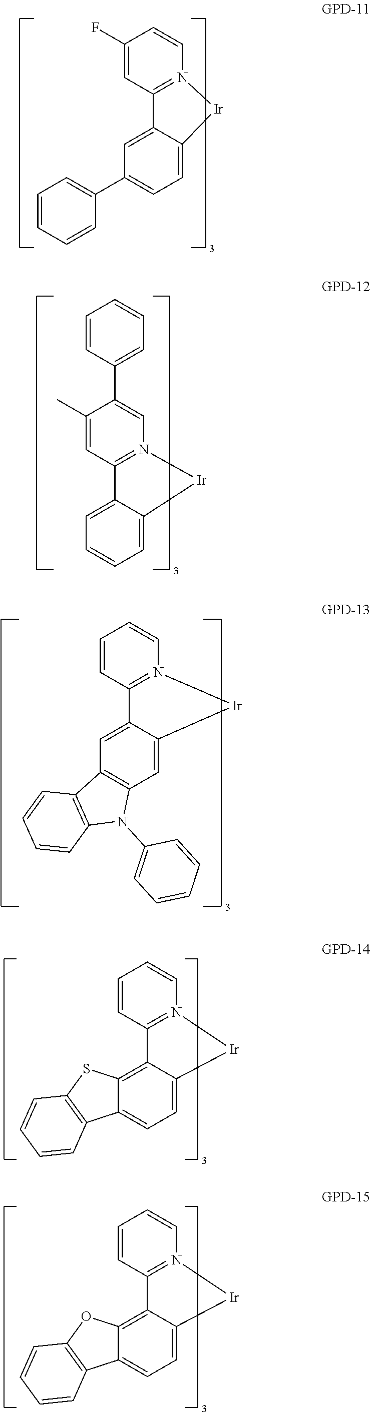

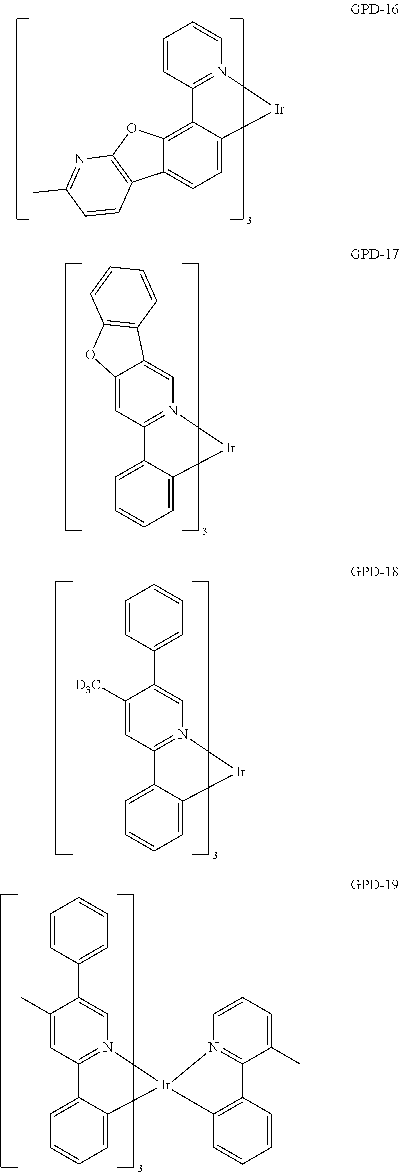

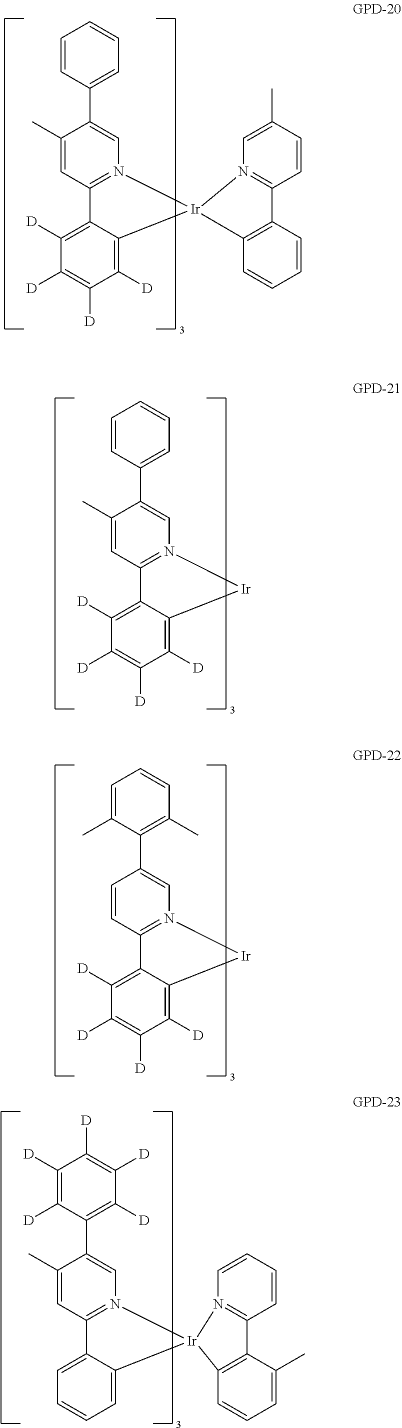

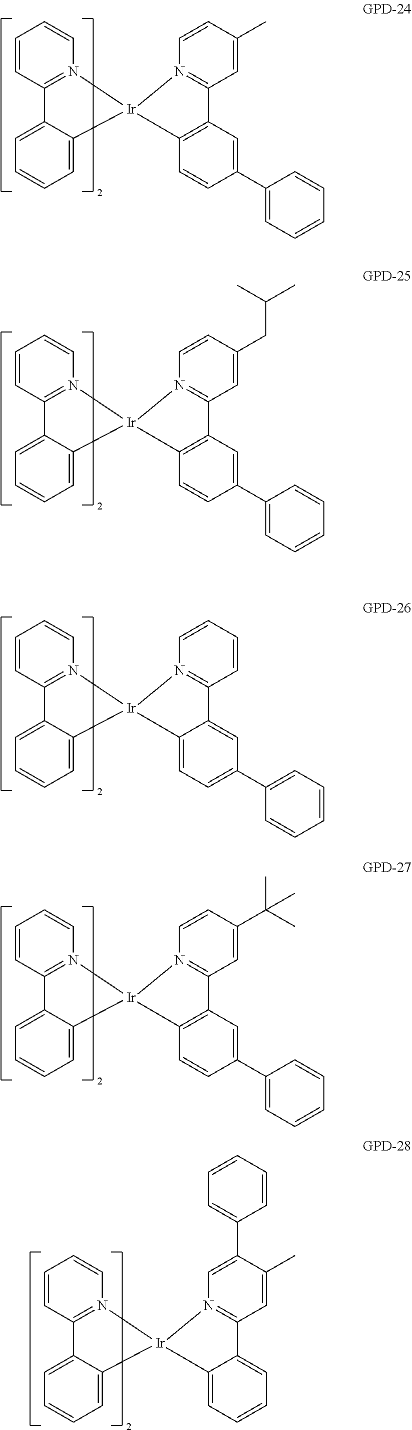

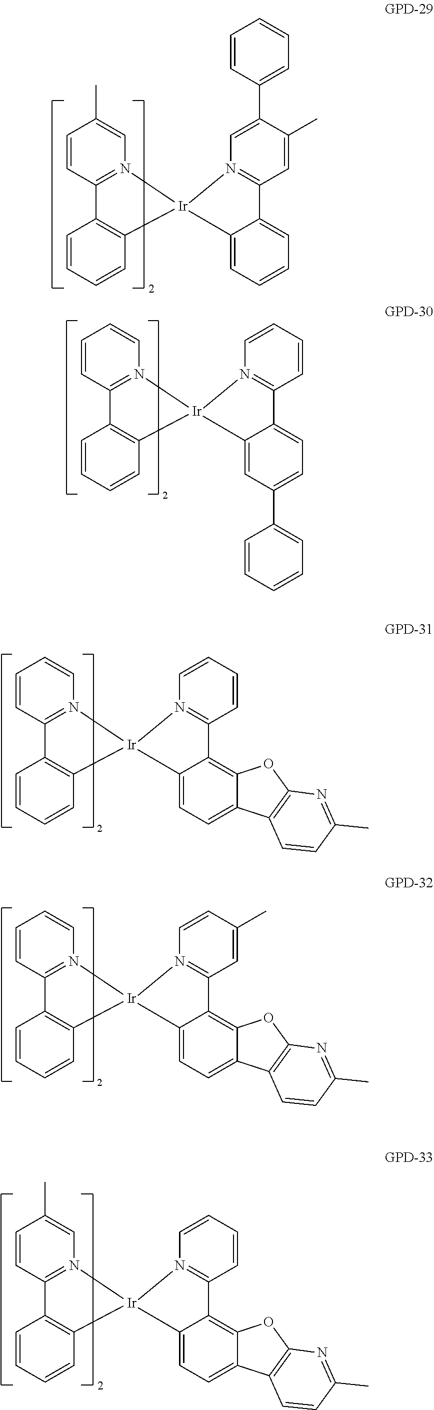

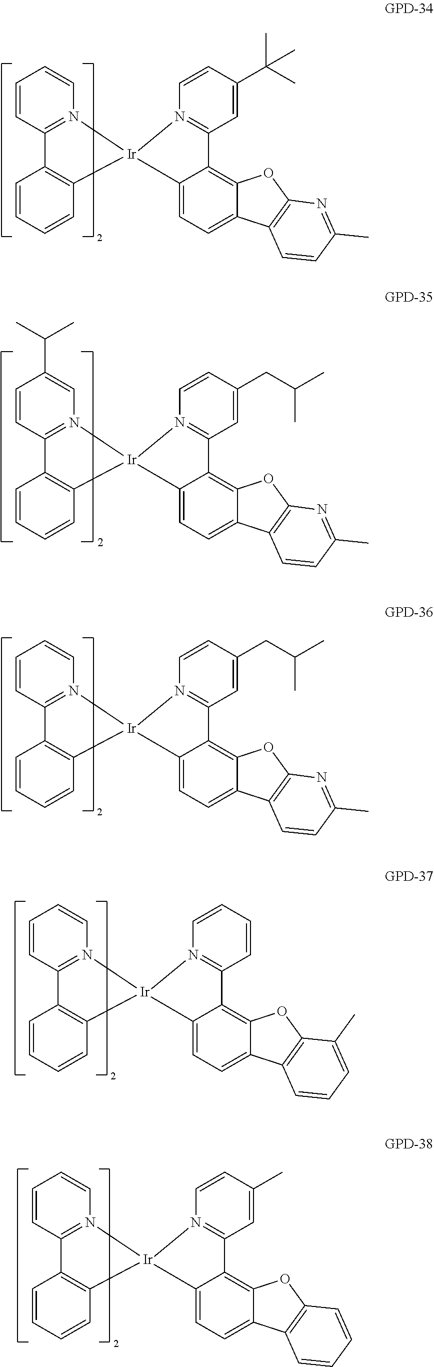

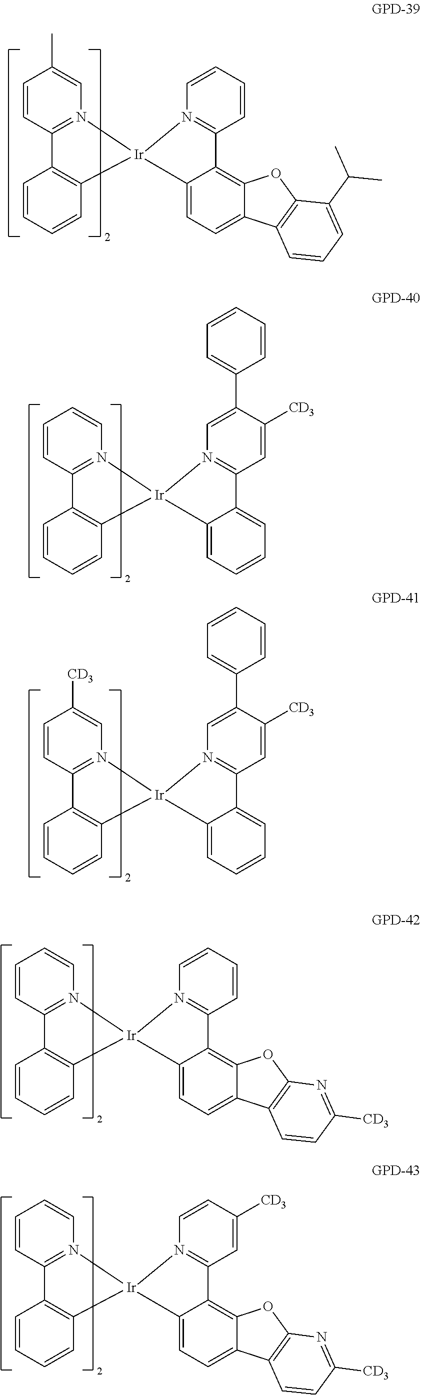

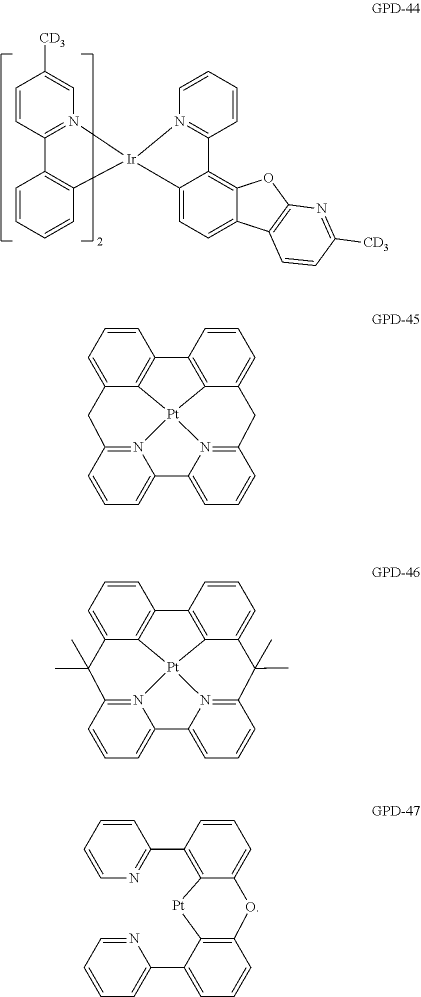

Where D is deuterium.

In one aspect of the disclosure, the phosphorescent electroluminescence technology is adopted for the light emitting layer. The phosphorescent dopant of the light emitting layer thereof can be selected from the group consisting of, but not limited to one or a combination of more of RPD-1 to RPD-28 as listed below.

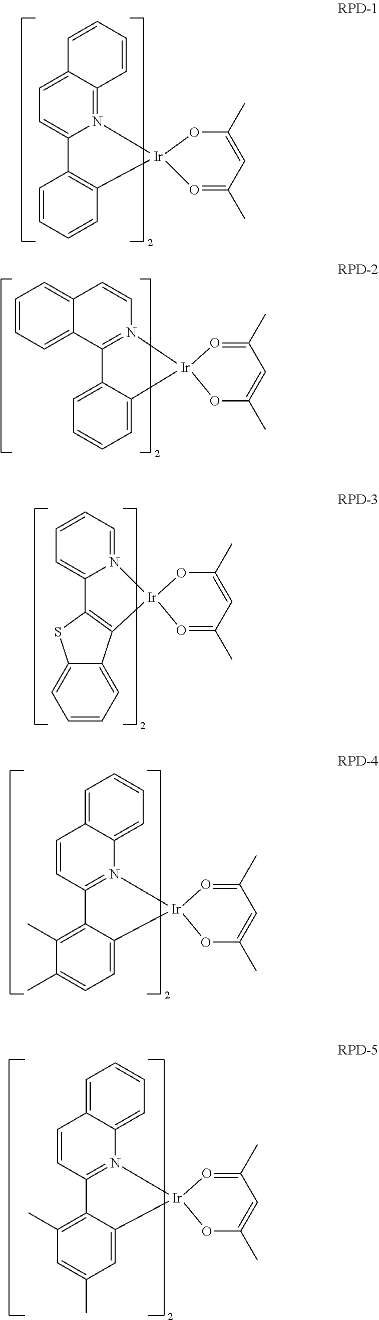

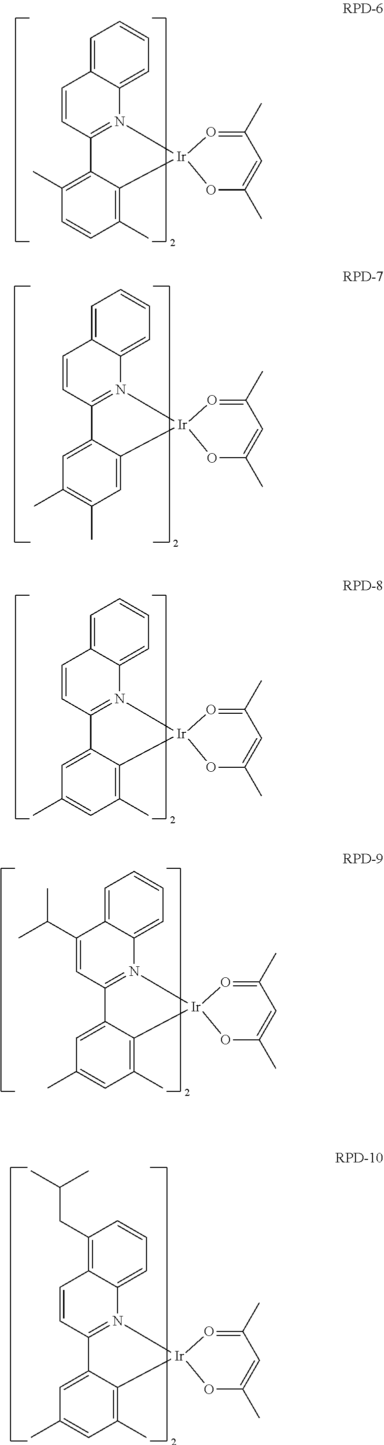

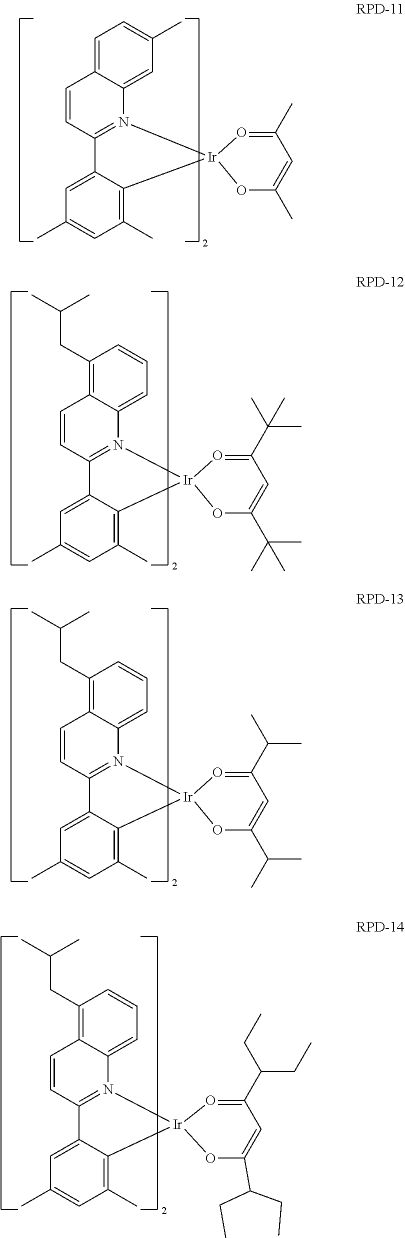

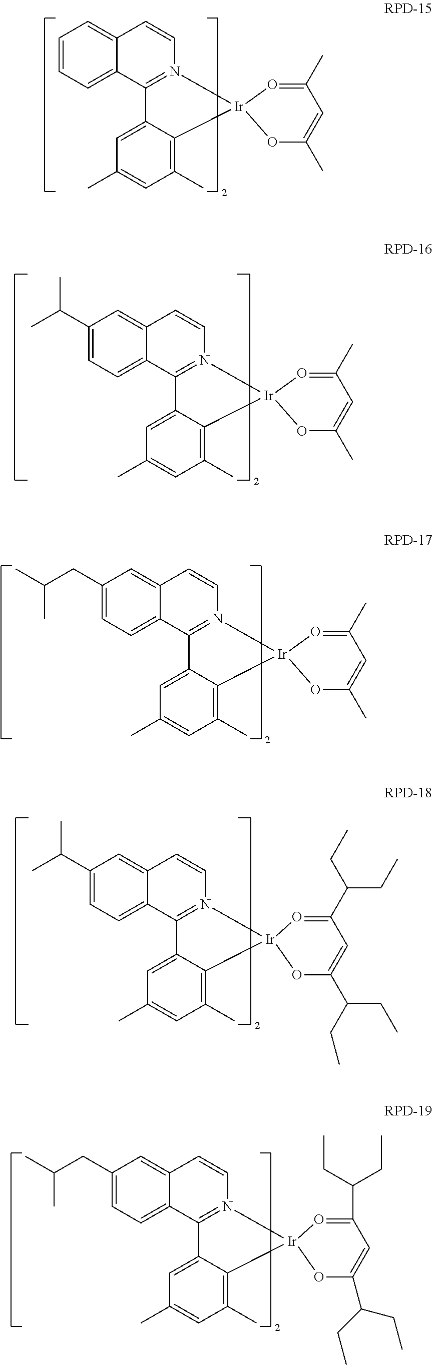

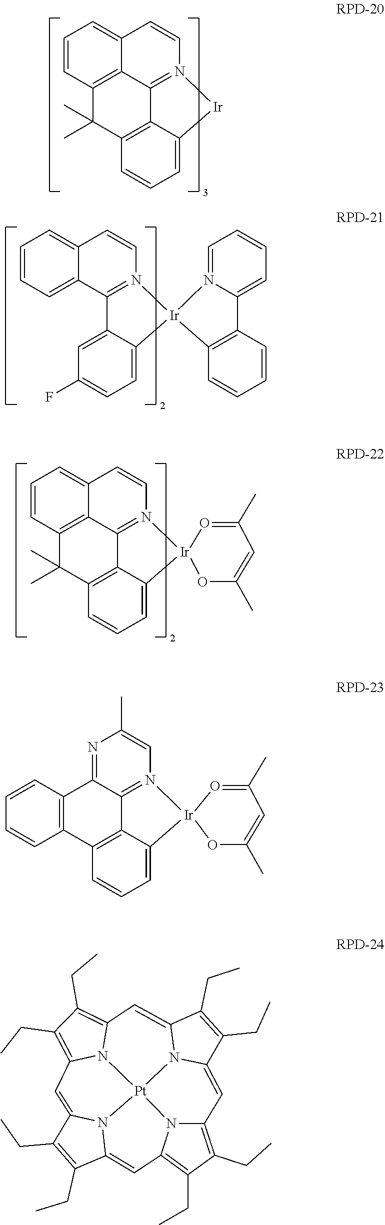

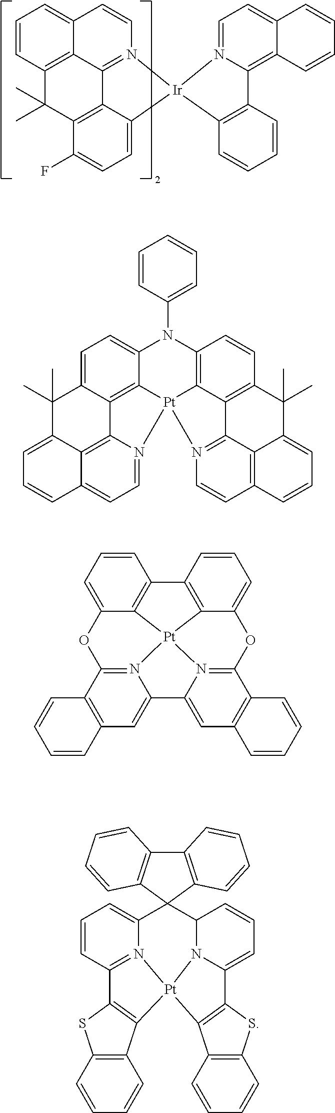

In one aspect of the disclosure, the phosphorescent electroluminescence technology is adopted for the light emitting layer. The phosphorescent dopant of the light emitting layer thereof can be selected from the group consisting of, but not limited to one or a combination of more of YPD-1 to YPD-11 as listed below.

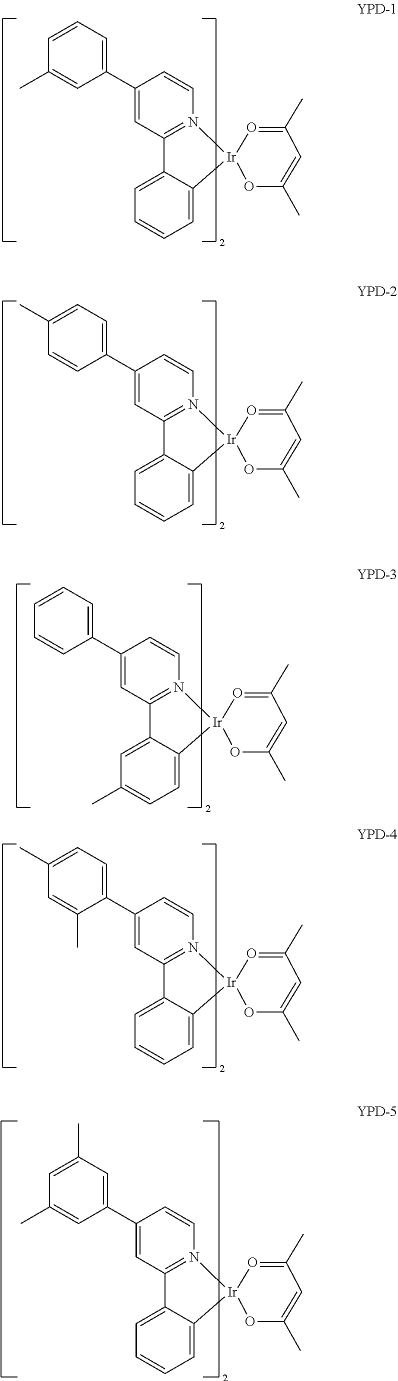

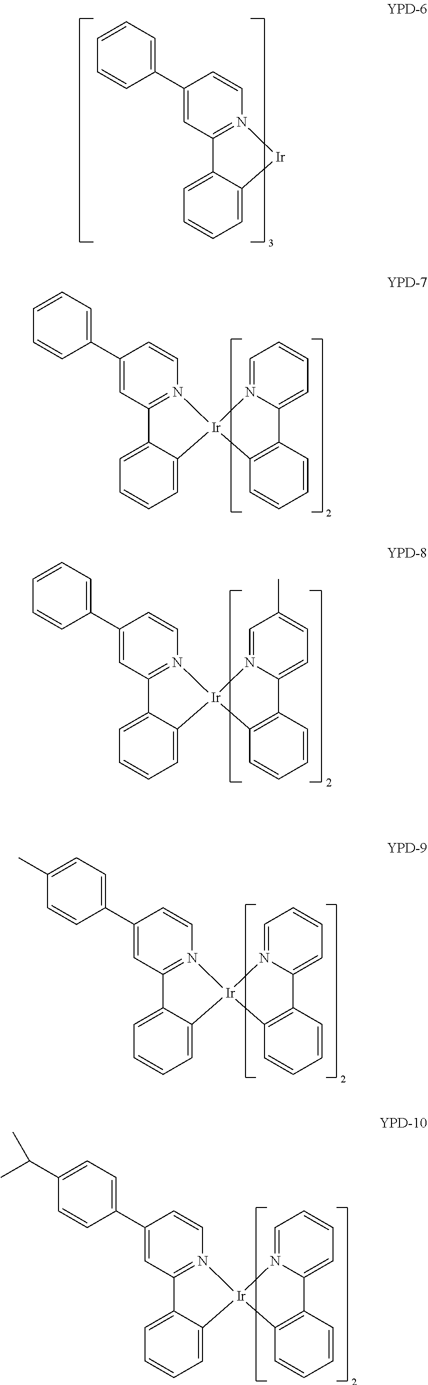

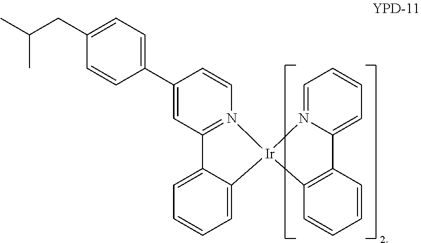

In one aspect of the disclosure, the thermally activated delayed fluorescent light emitting technology is adopted for the light emitting layer. The fluorescent dopant of the light emitting layer thereof can be selected from the group consisting of, but not limited to one or a combination of more of TDE-1 to TDE-39 as listed below.

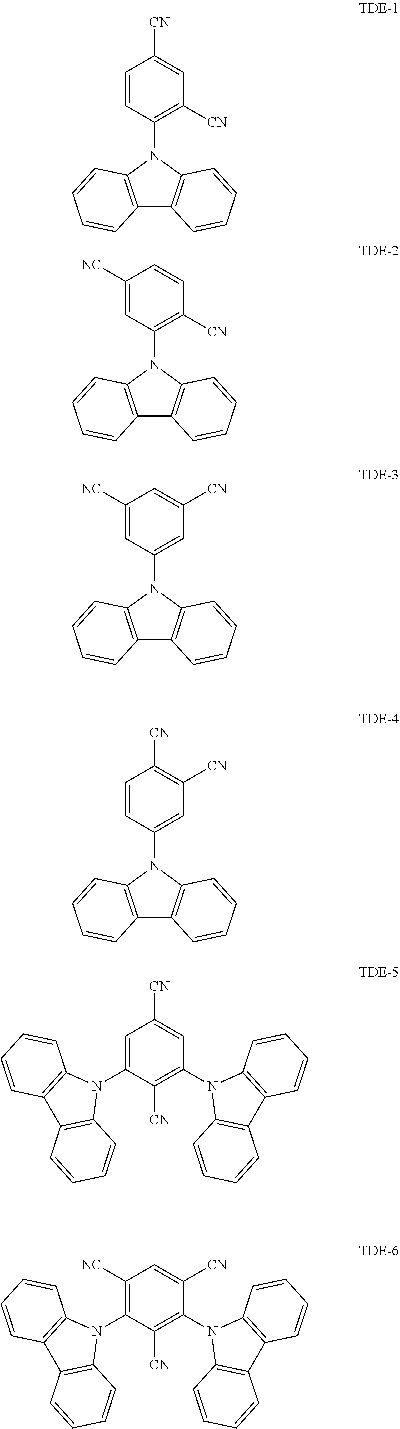

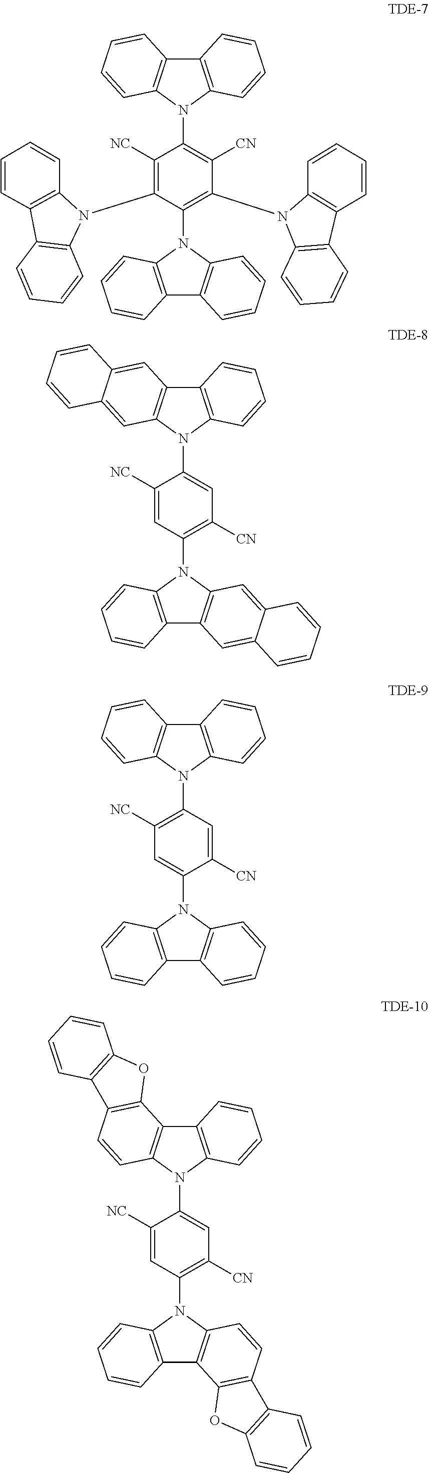

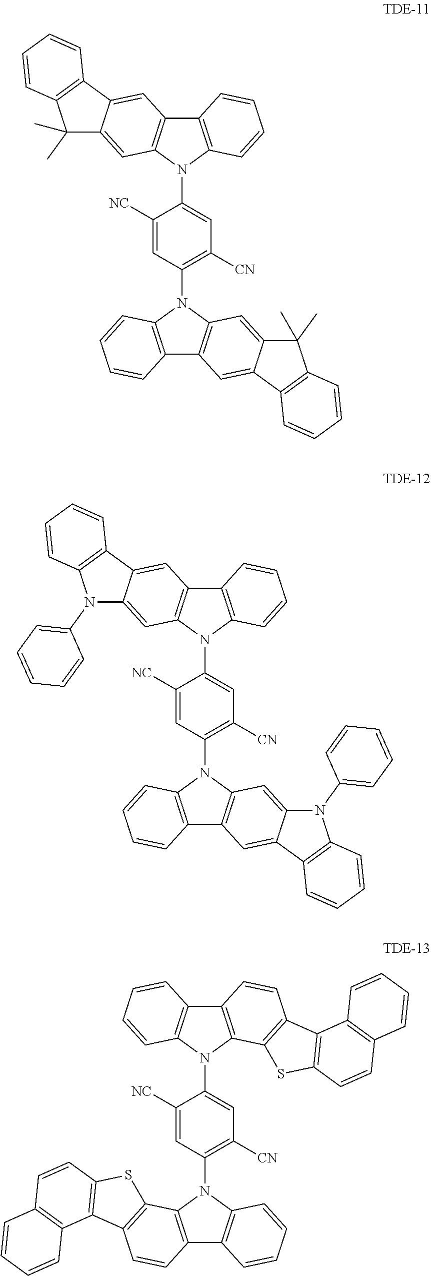 [SMILES: CC1(C)c2ccccc2-c2cc3c4ccccc4n(-c4cc(C#N)c(-n5c6ccccc6c6cc7c(cc65)C(C)(C)c5ccccc5-7)cc4C#N)c3cc21 ; N#Cc1cc(-n2c3ccccc3c3cc4c5ccccc5n(-c5ccccc5)c4cc32)c(C#N)cc1-n1c2ccccc2c2cc3c4ccccc4n(-c4ccccc4)c3cc21 ; N#Cc1cc(-n2c3ccccc3c3ccc4c(sc5ccc6ccccc6c54)c32)c(C#N)cc1-n1c2ccccc2c2ccc3c(sc4ccc5ccccc5c43)c21]

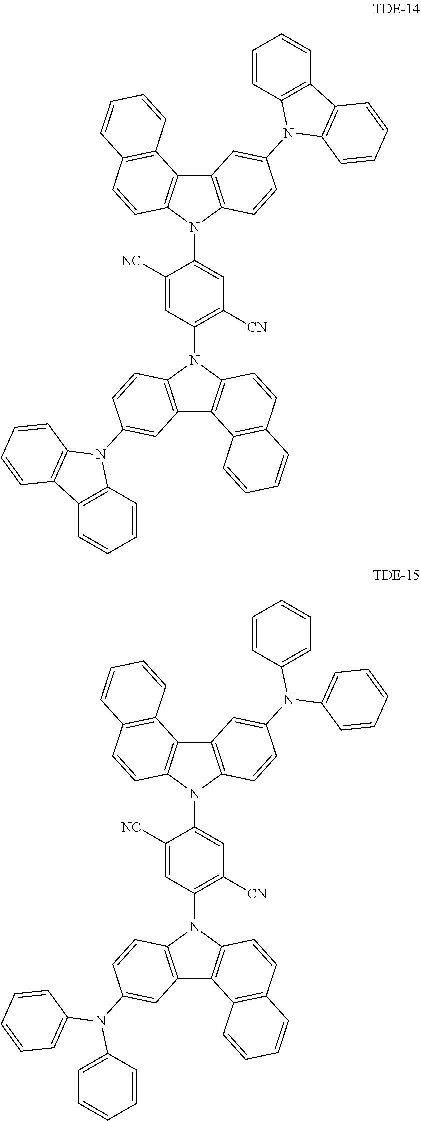 [SMILES: N#Cc1cc(-n2c3ccc(-n4c5ccccc5c5ccccc54)cc3c3c4ccccc4ccc32)c(C#N)cc1-n1c2ccc(-n3c4ccccc4c4ccccc43)cc2c2c3ccccc3ccc21 ; N#Cc1cc(-n2c3ccc(N(c4ccccc4)c4ccccc4)cc3c3c4ccccc4ccc32)c(C#N)cc1-n1c2ccc(N(c3ccccc3)c3ccccc3)cc2c2c3ccccc3ccc21]

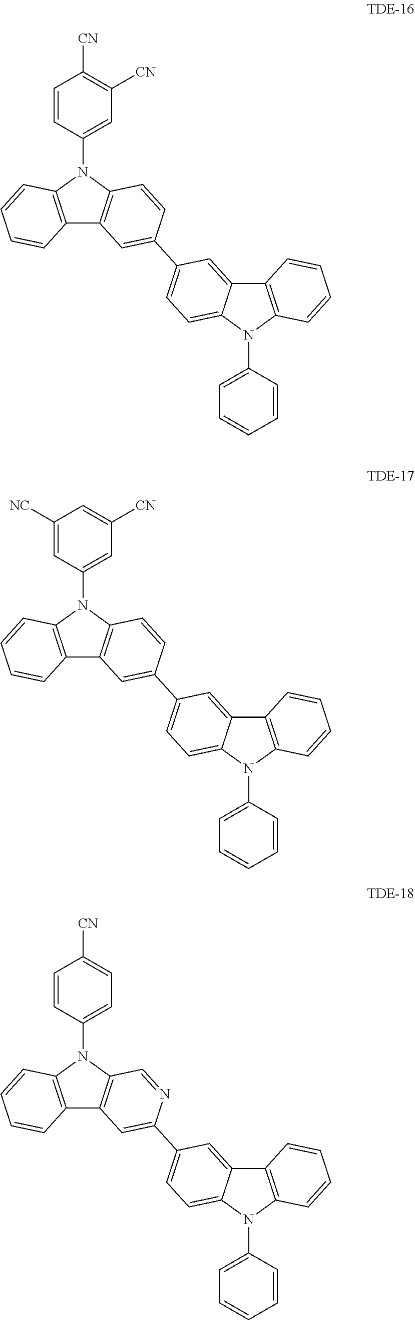 [SMILES: N#Cc1ccc(-n2c3ccccc3c3cc(-c4ccc5c(c4)c4ccccc4n5-c4ccccc4)ccc32)cc1C#N ; N#Cc1cc(C#N)cc(-n2c3ccccc3c3cc(-c4ccc5c(c4)c4ccccc4n5-c4ccccc4)ccc32)c1 ; N#Cc1ccc(-n2c3ccccc3c3cc(-c4ccc5c(c4)c4ccccc4n5-c4ccccc4)ncc32)cc1]

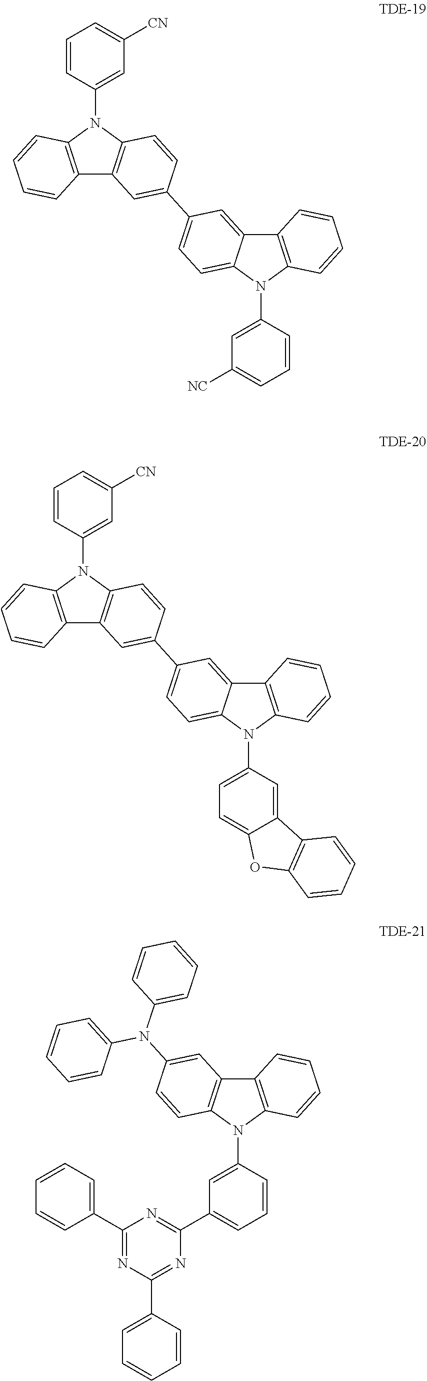 [SMILES: N#Cc1cccc(-n2c3ccccc3c3cc(-c4ccc5c(c4)c4ccccc4n5-c4cccc(C#N)c4)ccc32)c1 ; N#Cc1cccc(-n2c3ccccc3c3cc(-c4ccc5c(c4)c4ccccc4n5-c4ccc5oc6ccccc6c5c4)ccc32)c1 ; c1ccc(-c2nc(-c3ccccc3)nc(-c3cccc(-n4c5ccccc5c5cc(N(c6ccccc6)c6ccccc6)ccc54)c3)n2)cc1]

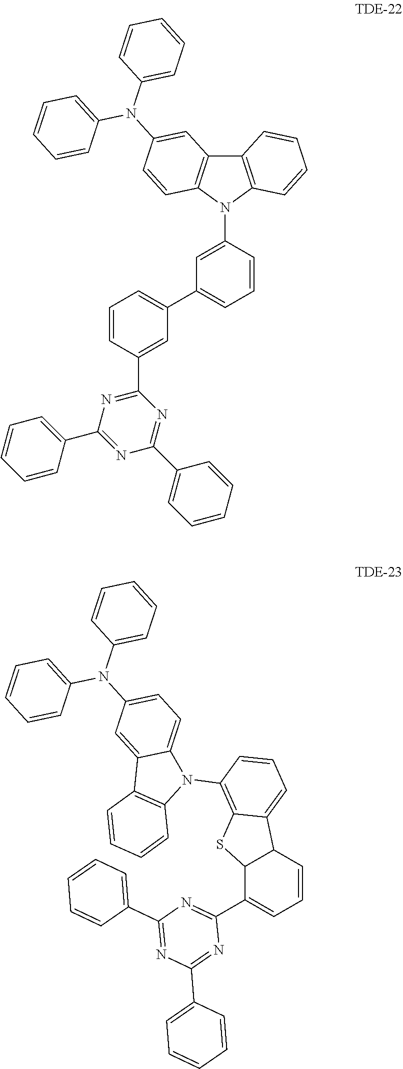 [SMILES: c1ccc(-c2nc(-c3ccccc3)nc(-c3cccc(-c4cccc(-n5c6ccccc6c6cc(N(c7ccccc7)c7ccccc7)ccc65)c4)c3)n2)cc1 ; C1=CC2c3cccc(-n4c5ccccc5c5cc(N(c6ccccc6)c6ccccc6)ccc54)c3SC2C(c2nc(-c3ccccc3)nc(-c3ccccc3)n2)=C1]

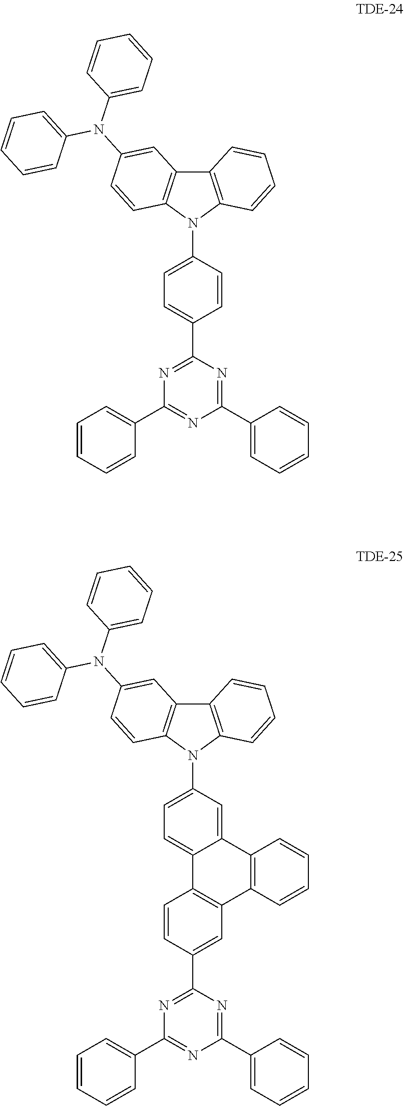 [SMILES: c1ccc(-c2nc(-c3ccccc3)nc(-c3ccc(-n4c5ccccc5c5cc(N(c6ccccc6)c6ccccc6)ccc54)cc3)n2)cc1 ; c1ccc(-c2nc(-c3ccccc3)nc(-c3ccc4c5ccc(-n6c7ccccc7c7cc(N(c8ccccc8)c8ccccc8)ccc76)cc5c5ccccc5c4c3)n2)cc1]

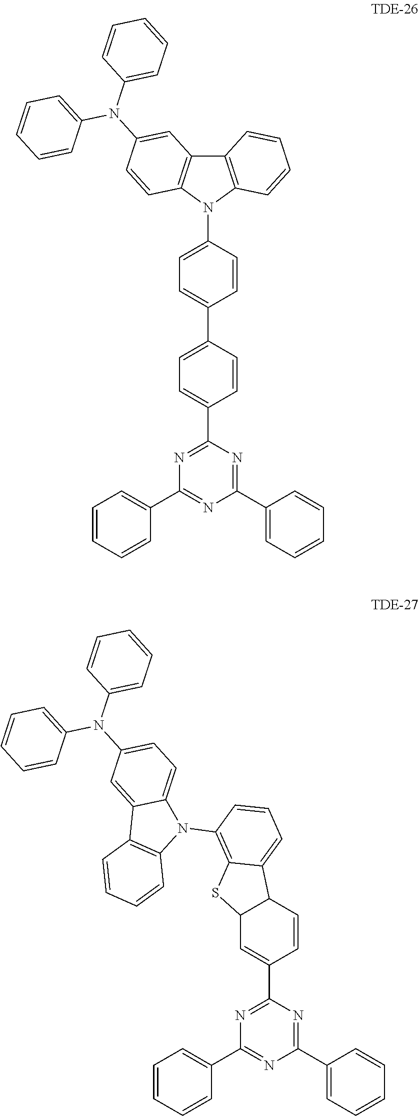 [SMILES: c1ccc(-c2nc(-c3ccccc3)nc(-c3ccc(-c4ccc(-n5c6ccccc6c6cc(N(c7ccccc7)c7ccccc7)ccc65)cc4)cc3)n2)cc1 ; C1=CC2c3cccc(-n4c5ccccc5c5cc(N(c6ccccc6)c6ccccc6)ccc54)c3SC2C=C1c1nc(-c2ccccc2)nc(-c2ccccc2)n1]

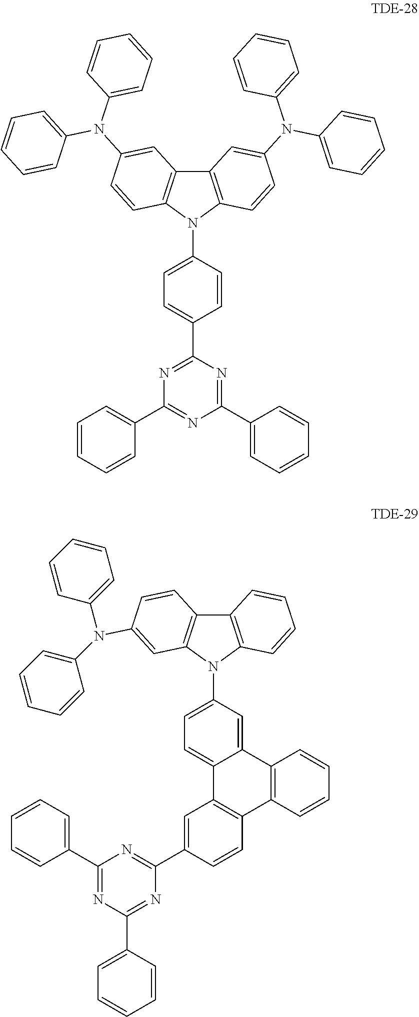 [SMILES: c1ccc(-c2nc(-c3ccccc3)nc(-c3ccc(-n4c5ccc(N(c6ccccc6)c6ccccc6)cc5c5cc(N(c6ccccc6)c6ccccc6)ccc54)cc3)n2)cc1 ; c1ccc(-c2nc(-c3ccccc3)nc(-c3ccc4c5ccccc5c5cc(-n6c7ccccc7c7ccc(N(c8ccccc8)c8ccccc8)cc76)ccc5c4c3)n2)cc1]

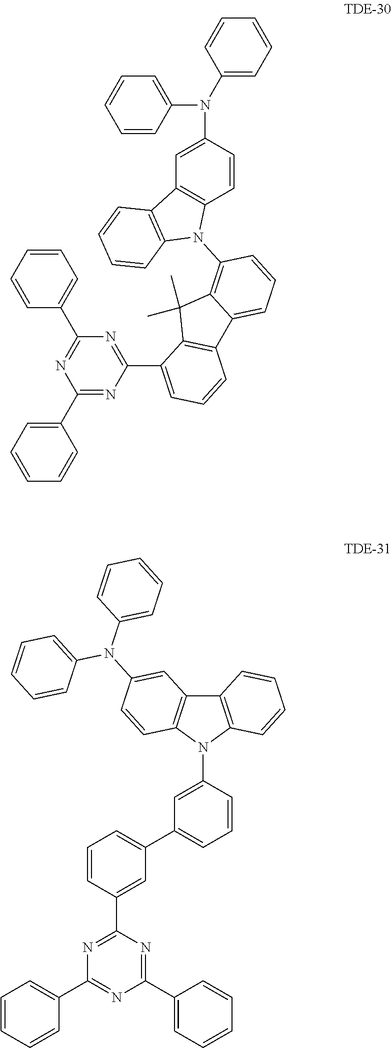 [SMILES: CC1(C)c2c(-c3nc(-c4ccccc4)nc(-c4ccccc4)n3)cccc2-c2cccc(-n3c4ccccc4c4cc(N(c5ccccc5)c5ccccc5)ccc43)c21 ; c1ccc(-c2nc(-c3ccccc3)nc(-c3cccc(-c4cccc(-n5c6ccccc6c6cc(N(c7ccccc7)c7ccccc7)ccc65)c4)c3)n2)cc1]

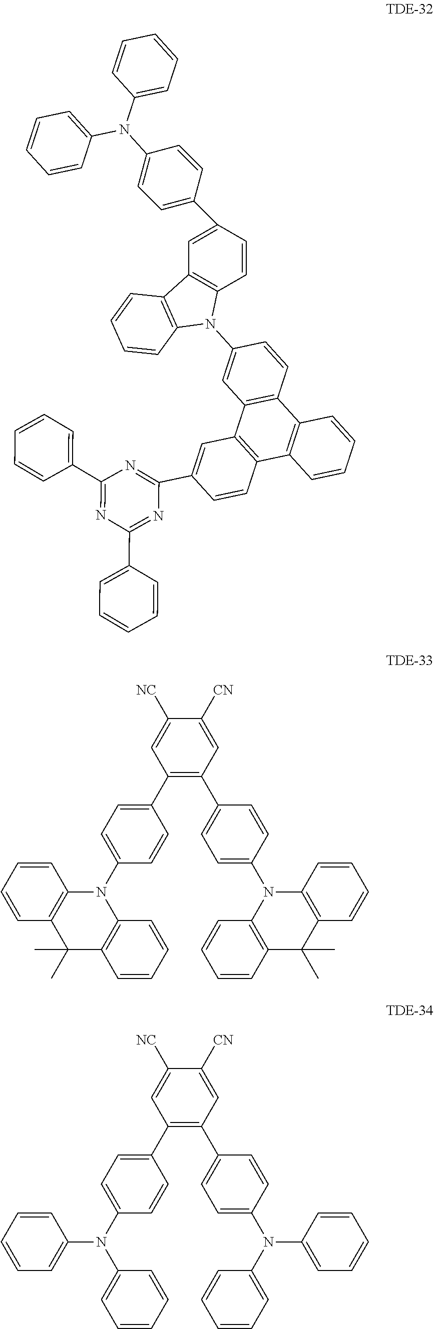 [SMILES: c1ccc(-c2nc(-c3ccccc3)nc(-c3ccc4c5ccccc5c5ccc(-n6c7ccccc7c7cc(-c8ccc(N(c9ccccc9)c9ccccc9)cc8)ccc76)cc5c4c3)n2)cc1 ; CC1(C)c2ccccc2N(c2ccc(-c3cc(C#N)c(C#N)cc3-c3ccc(N4c5ccccc5C(C)(C)c5ccccc54)cc3)cc2)c2ccccc21 ; N#Cc1cc(-c2ccc(N(c3ccccc3)c3ccccc3)cc2)c(-c2ccc(N(c3ccccc3)c3ccccc3)cc2)cc1C#N]

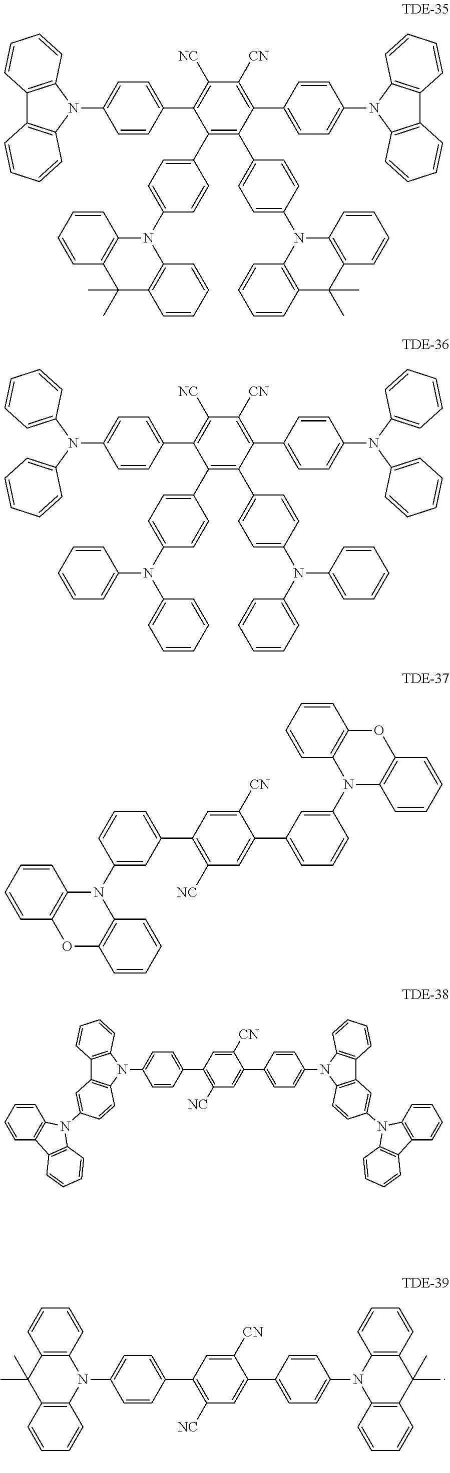

In one aspect of the disclosure, the thermally activated delayed fluorescent light emitting technology is adopted for the light emitting layer. The host material of the light emitting layer thereof is selected from the group consisting of, but not limited to one or a combination of more of TDH1 to TDH24.

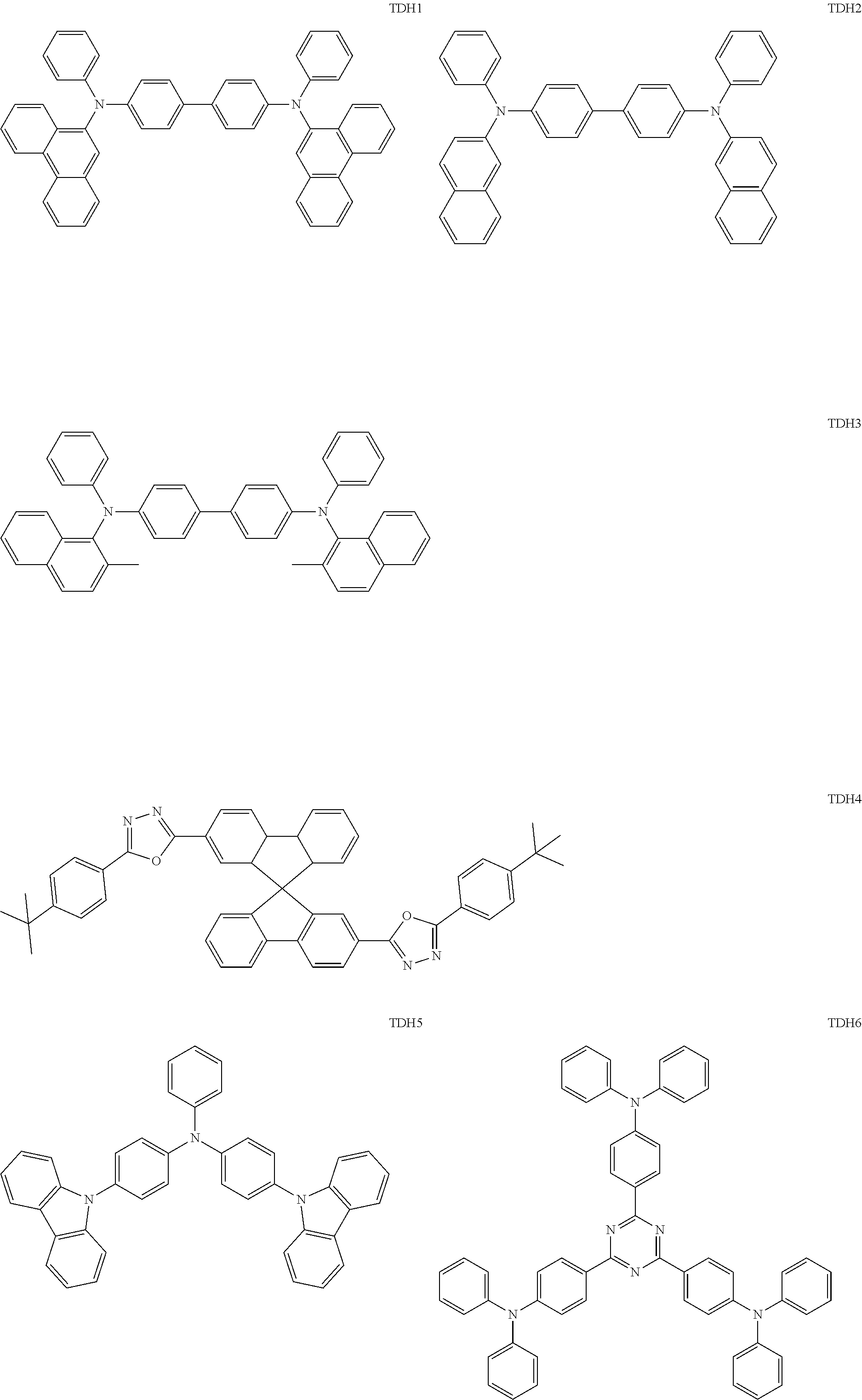

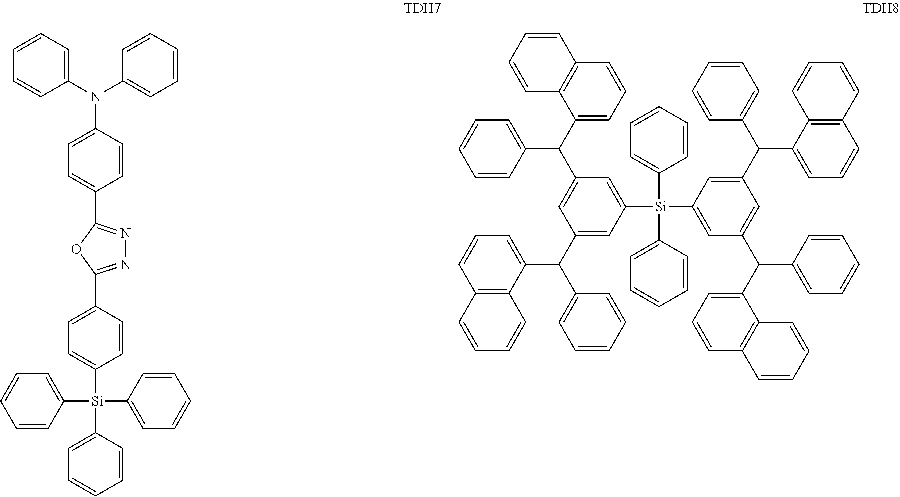 [SMILES: c1ccc(N(c2ccccc2)c2ccc(-c3nnc(-c4ccc([Si](c5ccccc5)(c5ccccc5)c5ccccc5)cc4)o3)cc2)cc1 ; c1ccc(C(c2cc(C(c3ccccc3)c3cccc4ccccc34)cc([Si](c3ccccc3)(c3ccccc3)c3cc(C(c4ccccc4)c4cccc5ccccc45)cc(C(c4ccccc4)c4cccc5ccccc45)c3)c2)c2cccc3ccccc23)cc1]

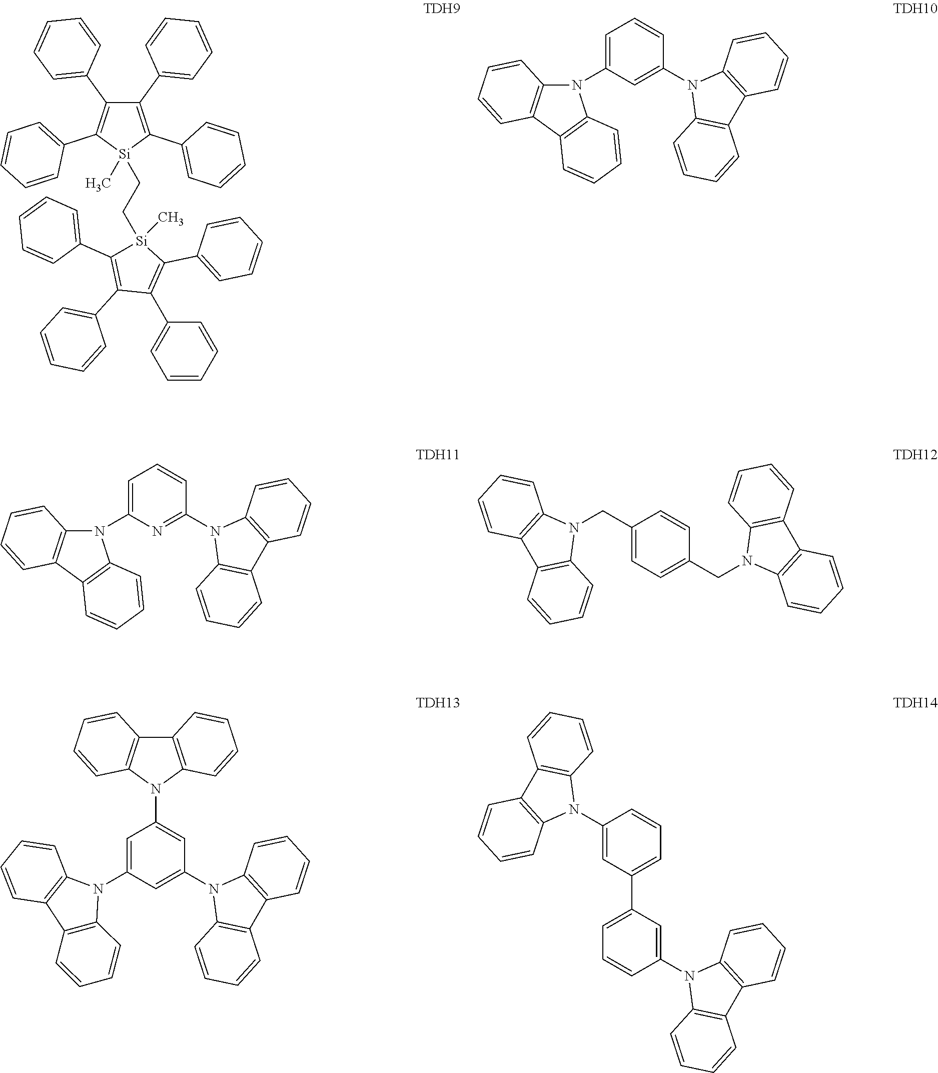 [SMILES: C[Si]1(CC[Si]2(C)C(c3ccccc3)=C(c3ccccc3)C(c3ccccc3)=C2c2ccccc2)C(c2ccccc2)=C(c2ccccc2)C(c2ccccc2)=C1c1ccccc1 ; c1cc(-n2c3ccccc3c3ccccc32)cc(-n2c3ccccc3c3ccccc32)c1 ; c1cc(-n2c3ccccc3c3ccccc32)nc(-n2c3ccccc3c3ccccc32)c1 ; c1ccc2c(c1)c1ccccc1n2Cc1ccc(Cn2c3ccccc3c3ccccc32)cc1 ; c1ccc2c(c1)c1ccccc1n2-c1cc(-n2c3ccccc3c3ccccc32)cc(-n2c3ccccc3c3ccccc32)c1 ; c1cc(-c2cccc(-n3c4ccccc4c4ccccc43)c2)cc(-n2c3ccccc3c3ccccc32)c1]

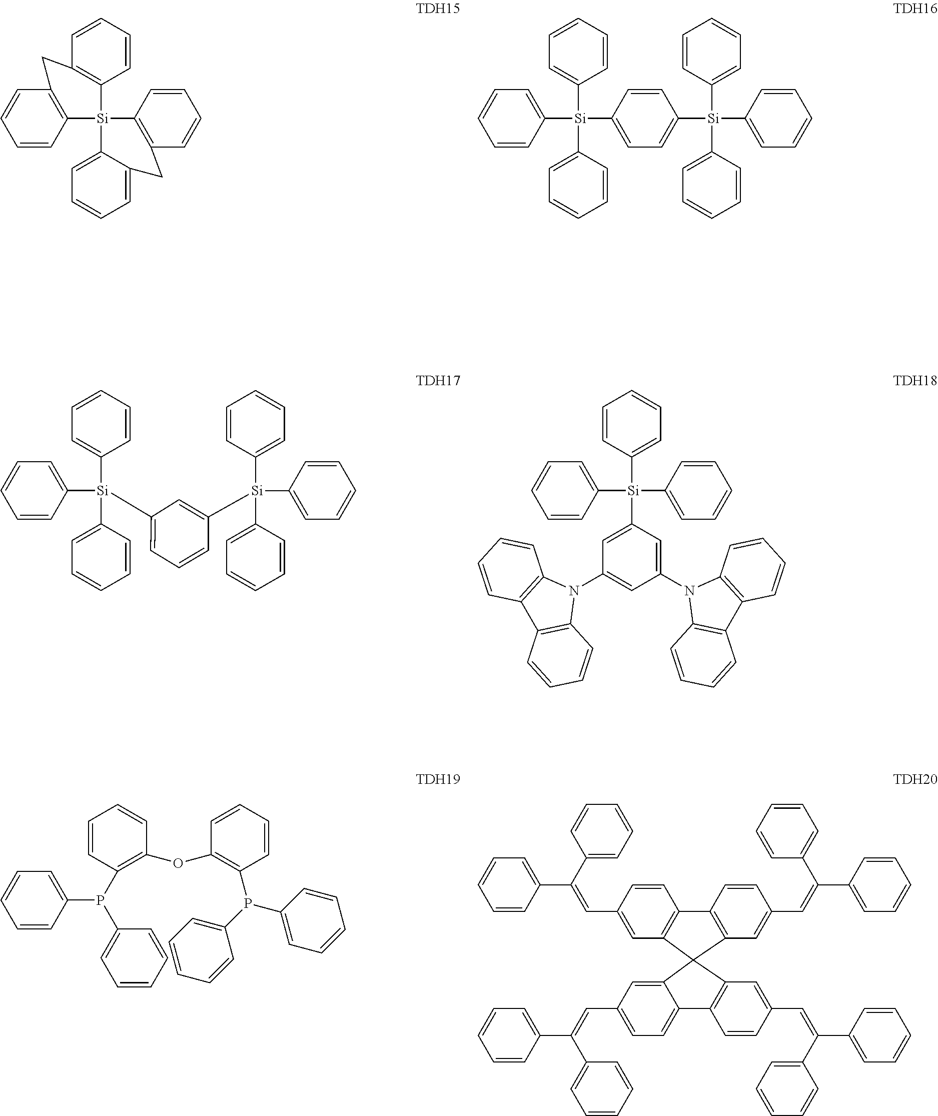

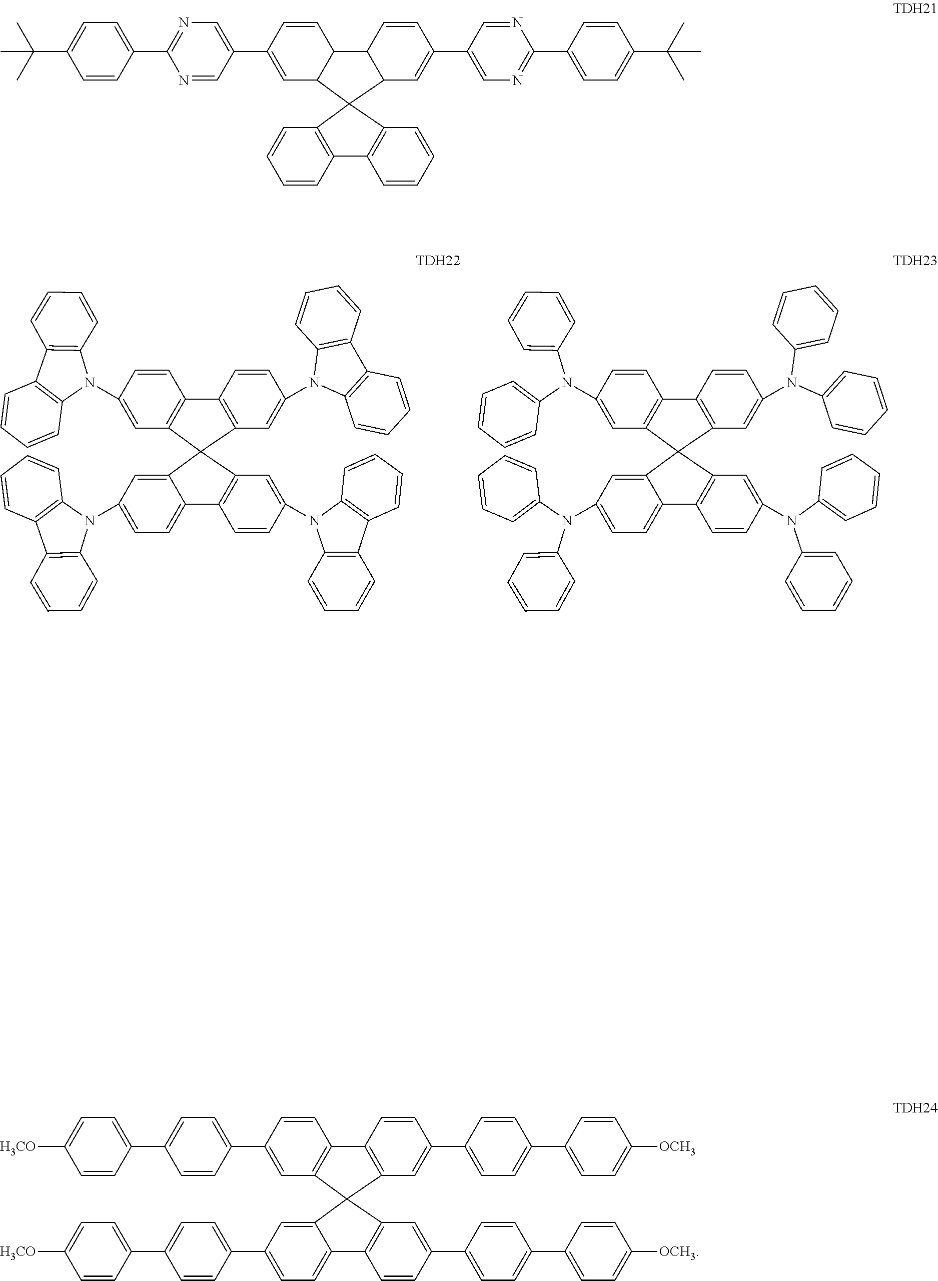

The OLED organic material layer may also include an electron transporting region between the light emitting layer and the cathode. The electron transporting region can be an electron transporting layer (ETL) with a single-layer structure, including a single-layer electron transporting layer containing only one compound and a single-layer electron transporting layer containing multiple compounds. The electron transporting region may also be a multilayer structure comprising at least one of an electron injection layer (EIL), an electron transporting layer (ETL), and a hole blocking layer (HBL).

In one aspect of the disclosure, the electron transporting layer material can be selected from the group consisting of, but not limited to one or a combination of more of ET-1 to ET-57 as listed below.

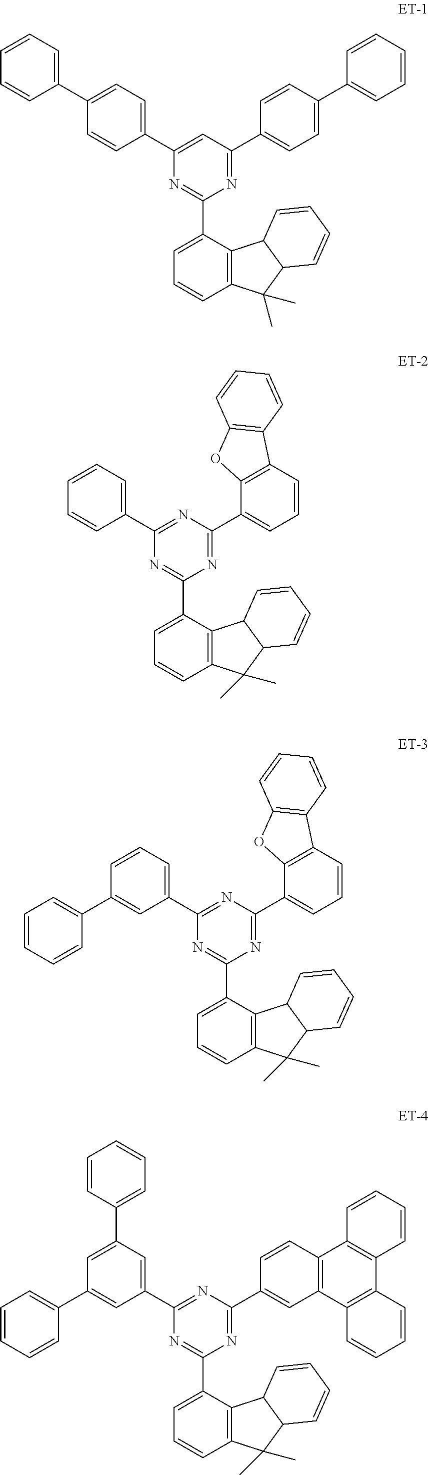 [SMILES: CC1(C)c2cccc(-c3nc(-c4ccc(-c5ccccc5)cc4)cc(-c4ccc(-c5ccccc5)cc4)n3)c2C2C=CC=CC21 ; CC1(C)c2cccc(-c3nc(-c4ccccc4)nc(-c4cccc5c4oc4ccccc45)n3)c2C2C=CC=CC21 ; CC1(C)c2cccc(-c3nc(-c4cccc(-c5ccccc5)c4)nc(-c4cccc5c4oc4ccccc45)n3)c2C2C=CC=CC21 ; CC1(C)c2cccc(-c3nc(-c4cc(-c5ccccc5)cc(-c5ccccc5)c4)nc(-c4ccc5c6ccccc6c6ccccc6c5c4)n3)c2C2C=CC=CC21]

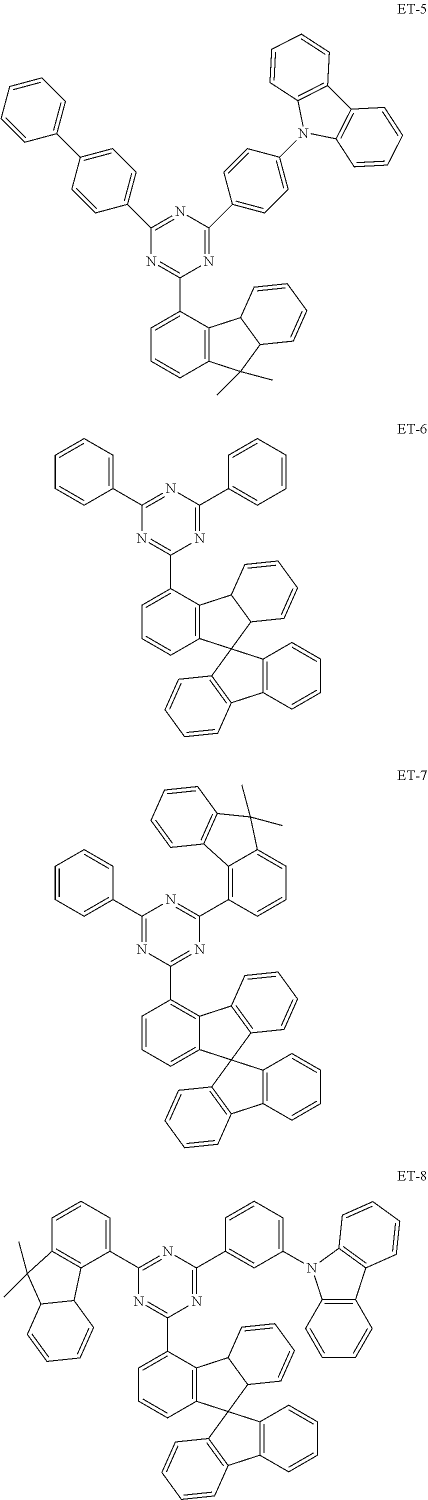 [SMILES: CC1(C)c2cccc(-c3nc(-c4ccc(-c5ccccc5)cc4)nc(-c4ccc(-n5c6ccccc6c6ccccc65)cc4)n3)c2C2C=CC=CC21 ; C1=CC2c3c(-c4nc(-c5ccccc5)nc(-c5ccccc5)n4)cccc3C3(c4ccccc4-c4ccccc43)C2C=C1 ; CC1(C)c2ccccc2-c2c(-c3nc(-c4ccccc4)nc(-c4cccc5c4-c4ccccc4C54c5ccccc5-c5ccccc54)n3)cccc21 ; CC1(C)c2cccc(-c3nc(-c4cccc(-n5c6ccccc6c6ccccc65)c4)nc(-c4cccc5c4C4C=CC=CC4C54c5ccccc5-c5ccccc54)n3)c2C2C=CC=CC21]

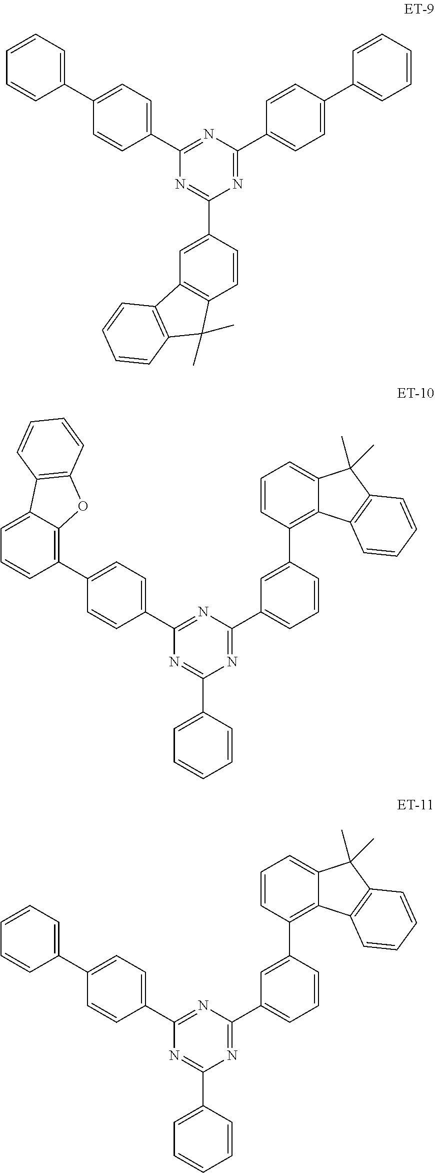 [SMILES: CC1(C)c2ccccc2-c2cc(-c3nc(-c4ccc(-c5ccccc5)cc4)nc(-c4ccc(-c5ccccc5)cc4)n3)ccc21 ; CC1(C)c2ccccc2-c2c(-c3cccc(-c4nc(-c5ccccc5)nc(-c5ccc(-c6cccc7c6oc6ccccc67)cc5)n4)c3)cccc21 ; CC1(C)c2ccccc2-c2c(-c3cccc(-c4nc(-c5ccccc5)nc(-c5ccc(-c6ccccc6)cc5)n4)c3)cccc21]

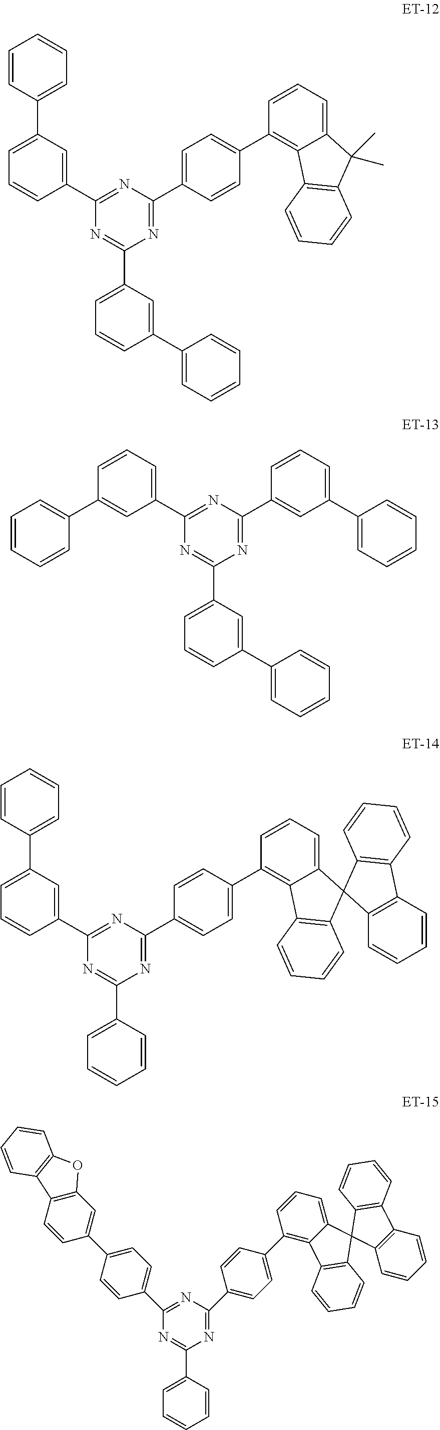 [SMILES: CC1(C)c2ccccc2-c2c(-c3ccc(-c4nc(-c5cccc(-c6ccccc6)c5)nc(-c5cccc(-c6ccccc6)c5)n4)cc3)cccc21 ; c1ccc(-c2cccc(-c3nc(-c4cccc(-c5ccccc5)c4)nc(-c4cccc(-c5ccccc5)c4)n3)c2)cc1 ; c1ccc(-c2cccc(-c3nc(-c4ccccc4)nc(-c4ccc(-c5cccc6c5-c5ccccc5C65c6ccccc6-c6ccccc65)cc4)n3)c2)cc1 ; c1ccc(-c2nc(-c3ccc(-c4ccc5c(c4)oc4ccccc45)cc3)nc(-c3ccc(-c4cccc5c4-c4ccccc4C54c5ccccc5-c5ccccc54)cc3)n2)cc1]

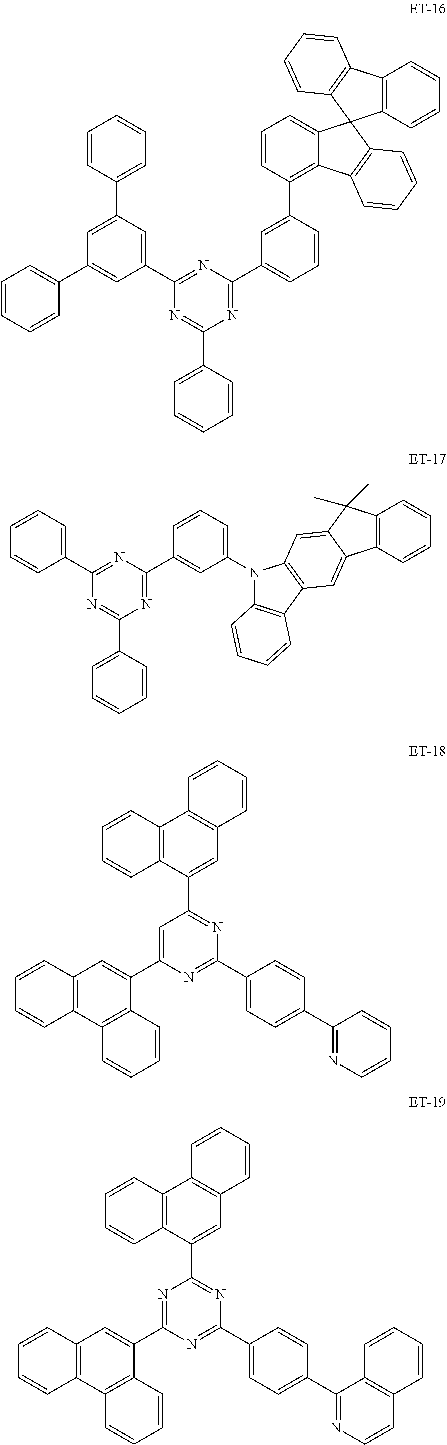 [SMILES: c1ccc(-c2cc(-c3ccccc3)cc(-c3nc(-c4ccccc4)nc(-c4cccc(-c5cccc6c5-c5ccccc5C65c6ccccc6-c6ccccc65)c4)n3)c2)cc1 ; CC1(C)c2ccccc2-c2cc3c4ccccc4n(-c4cccc(-c5nc(-c6ccccc6)nc(-c6ccccc6)n5)c4)c3cc21 ; c1ccc(-c2ccc(-c3nc(-c4cc5ccccc5c5ccccc45)cc(-c4cc5ccccc5c5ccccc45)n3)cc2)nc1 ; c1ccc2c(-c3ccc(-c4nc(-c5cc6ccccc6c6ccccc56)nc(-c5cc6ccccc6c6ccccc56)n4)cc3)nccc2c1]

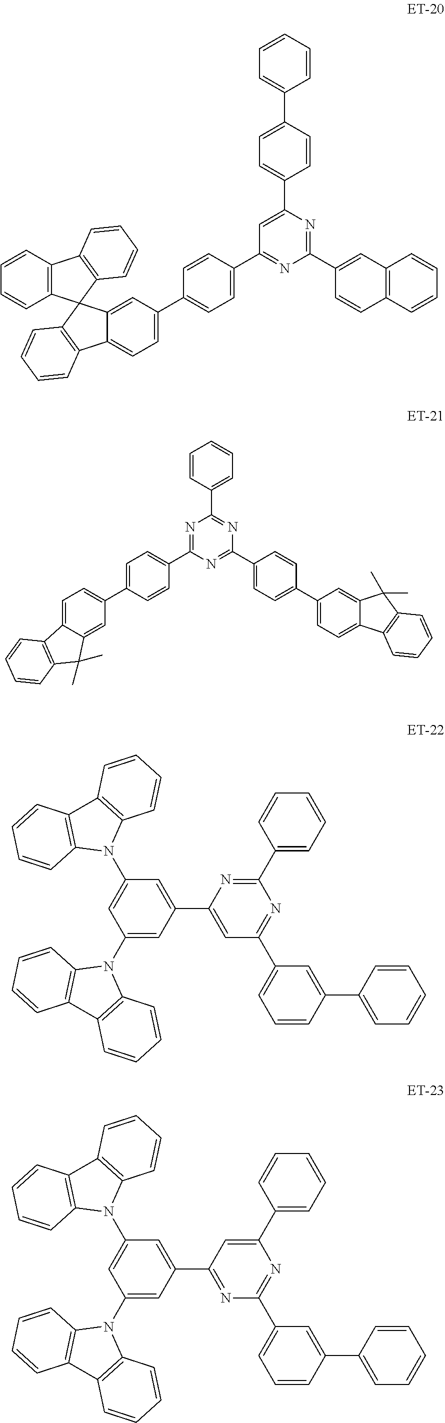 [SMILES: c1ccc(-c2ccc(-c3cc(-c4ccc(-c5ccc6c(c5)C5(c7ccccc7-c7ccccc75)c5ccccc5-6)cc4)nc(-c4ccc5ccccc5c4)n3)cc2)cc1 ; CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2cccc(-c3cc(-c4cc(-n5c6ccccc6c6ccccc65)cc(-n5c6ccccc6c6ccccc65)c4)nc(-c4ccccc4)n3)c2)cc1 ; c1ccc(-c2cccc(-c3nc(-c4ccccc4)cc(-c4cc(-n5c6ccccc6c6ccccc65)cc(-n5c6ccccc6c6ccccc65)c4)n3)c2)cc1]

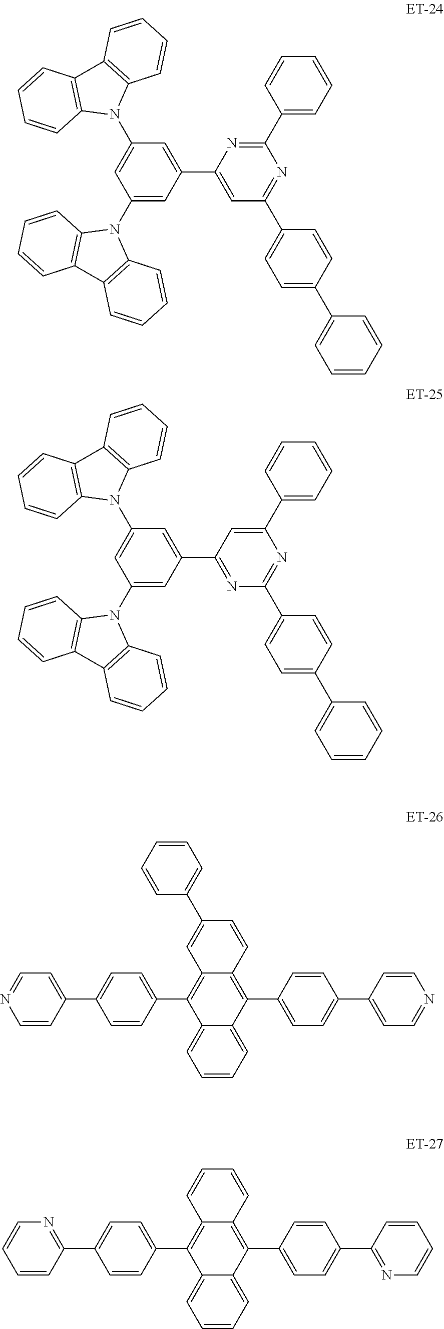 [SMILES: c1ccc(-c2ccc(-c3cc(-c4cc(-n5c6ccccc6c6ccccc65)cc(-n5c6ccccc6c6ccccc65)c4)nc(-c4ccccc4)n3)cc2)cc1 ; c1ccc(-c2ccc(-c3nc(-c4ccccc4)cc(-c4cc(-n5c6ccccc6c6ccccc65)cc(-n5c6ccccc6c6ccccc65)c4)n3)cc2)cc1 ; c1ccc(-c2ccc3c(-c4ccc(-c5ccncc5)cc4)c4ccccc4c(-c4ccc(-c5ccncc5)cc4)c3c2)cc1 ; c1ccc(-c2ccc(-c3c4ccccc4c(-c4ccc(-c5ccccn5)cc4)c4ccccc34)cc2)nc1]

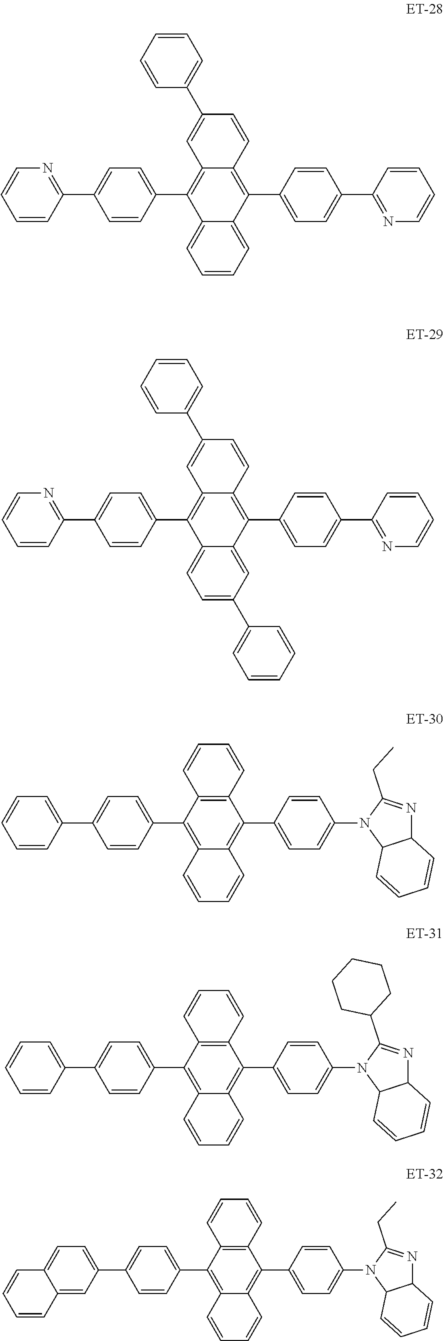 [SMILES: c1ccc(-c2ccc3c(-c4ccc(-c5ccccn5)cc4)c4ccccc4c(-c4ccc(-c5ccccn5)cc4)c3c2)cc1 ; c1ccc(-c2ccc3c(-c4ccc(-c5ccccn5)cc4)c4cc(-c5ccccc5)ccc4c(-c4ccc(-c5ccccn5)cc4)c3c2)cc1 ; CCC1=NC2C=CC=CC2N1c1ccc(-c2c3ccccc3c(-c3ccc(-c4ccccc4)cc3)c3ccccc23)cc1 ; C1=CC2N=C(C3CCCCC3)N(c3ccc(-c4c5ccccc5c(-c5ccc(-c6ccccc6)cc5)c5ccccc45)cc3)C2C=C1 ; CCC1=NC2C=CC=CC2N1c1ccc(-c2c3ccccc3c(-c3ccc(-c4ccc5ccccc5c4)cc3)c3ccccc23)cc1]

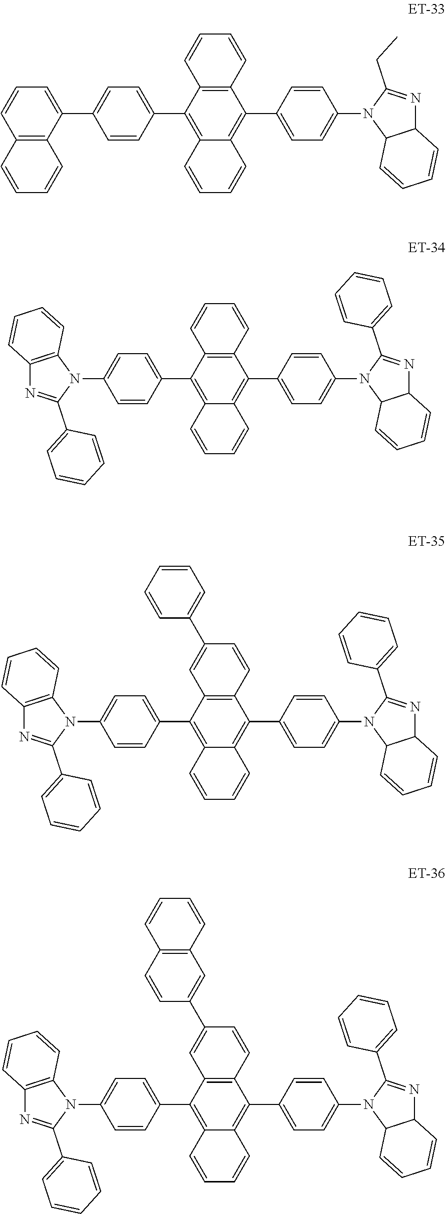 [SMILES: CCC1=NC2C=CC=CC2N1c1ccc(-c2c3ccccc3c(-c3ccc(-c4cccc5ccccc45)cc3)c3ccccc23)cc1 ; C1=CC2N=C(c3ccccc3)N(c3ccc(-c4c5ccccc5c(-c5ccc(-n6c(-c7ccccc7)nc7ccccc76)cc5)c5ccccc45)cc3)C2C=C1 ; C1=CC2N=C(c3ccccc3)N(c3ccc(-c4c5ccccc5c(-c5ccc(-n6c(-c7ccccc7)nc7ccccc76)cc5)c5cc(-c6ccccc6)ccc45)cc3)C2C=C1 ; C1=CC2N=C(c3ccccc3)N(c3ccc(-c4c5ccccc5c(-c5ccc(-n6c(-c7ccccc7)nc7ccccc76)cc5)c5cc(-c6ccc7ccccc7c6)ccc45)cc3)C2C=C1]

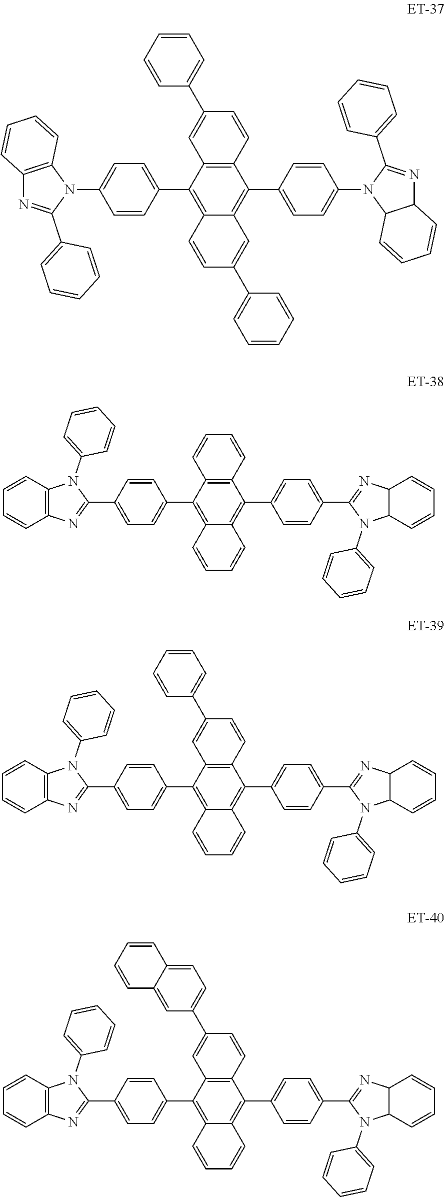 [SMILES: C1=CC2N=C(c3ccccc3)N(c3ccc(-c4c5ccc(-c6ccccc6)cc5c(-c5ccc(-n6c(-c7ccccc7)nc7ccccc76)cc5)c5ccc(-c6ccccc6)cc45)cc3)C2C=C1 ; C1=CC2N=C(c3ccc(-c4c5ccccc5c(-c5ccc(-c6nc7ccccc7n6-c6ccccc6)cc5)c5ccccc45)cc3)N(c3ccccc3)C2C=C1 ; C1=CC2N=C(c3ccc(-c4c5ccccc5c(-c5ccc(-c6nc7ccccc7n6-c6ccccc6)cc5)c5cc(-c6ccccc6)ccc45)cc3)N(c3ccccc3)C2C=C1 ; C1=CC2N=C(c3ccc(-c4c5ccccc5c(-c5ccc(-c6nc7ccccc7n6-c6ccccc6)cc5)c5cc(-c6ccc7ccccc7c6)ccc45)cc3)N(c3ccccc3)C2C=C1]

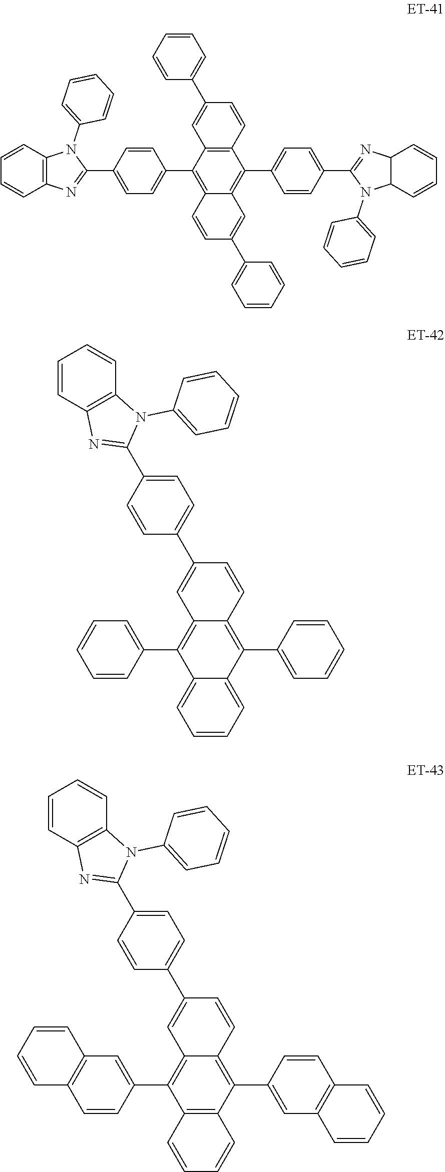 [SMILES: C1=CC2N=C(c3ccc(-c4c5ccc(-c6ccccc6)cc5c(-c5ccc(-c6nc7ccccc7n6-c6ccccc6)cc5)c5ccc(-c6ccccc6)cc45)cc3)N(c3ccccc3)C2C=C1 ; c1ccc(-c2c3ccccc3c(-c3ccccc3)c3cc(-c4ccc(-c5nc6ccccc6n5-c5ccccc5)cc4)ccc23)cc1 ; c1ccc(-n2c(-c3ccc(-c4ccc5c(-c6ccc7ccccc7c6)c6ccccc6c(-c6ccc7ccccc7c6)c5c4)cc3)nc3ccccc32)cc1]

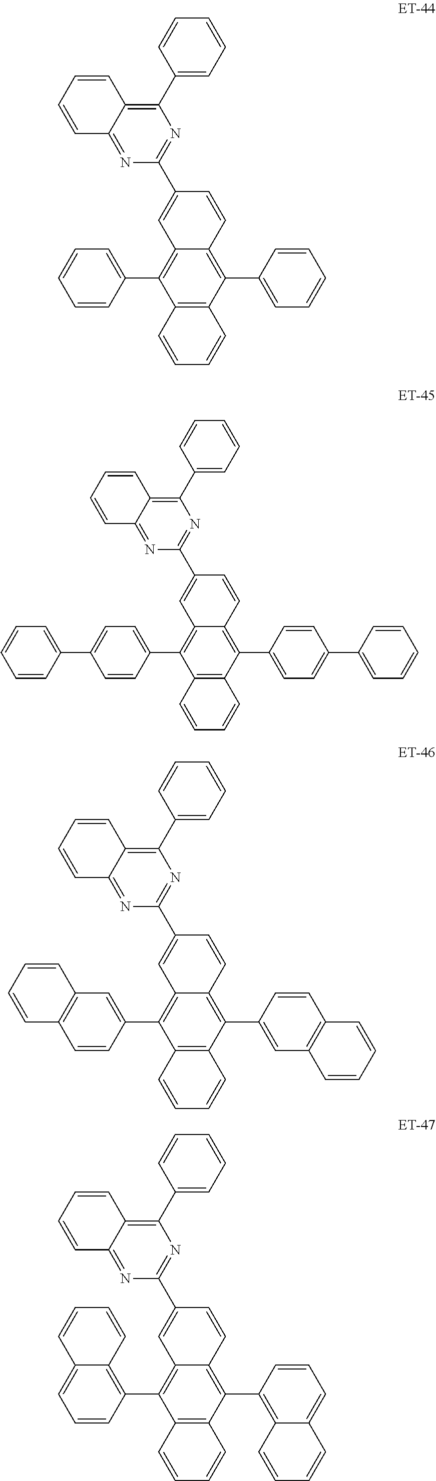

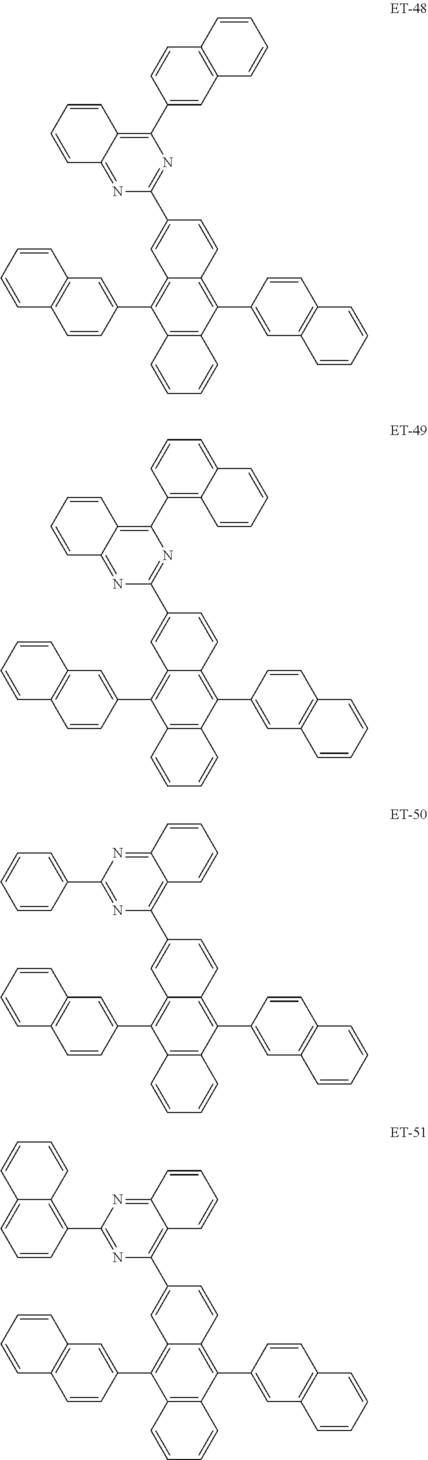

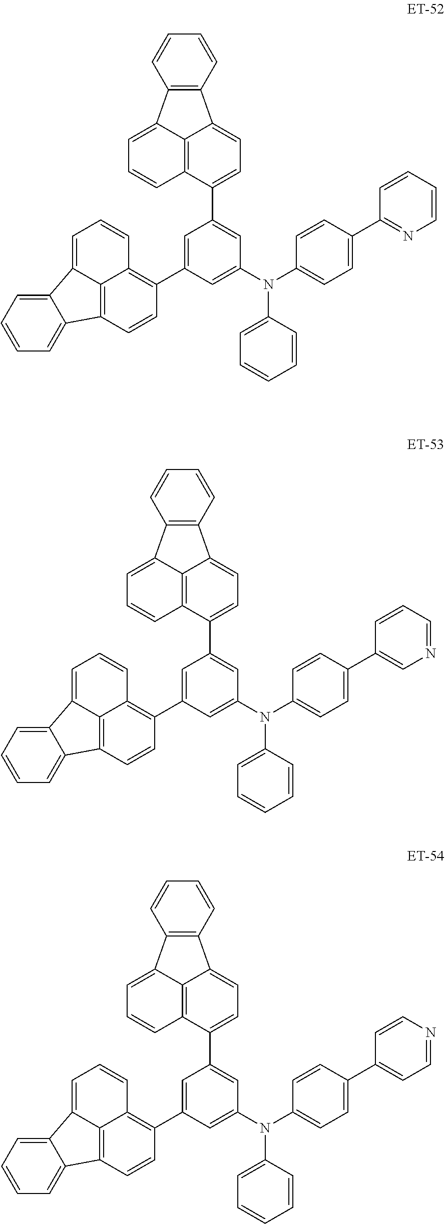 [SMILES: c1ccc(N(c2ccc(-c3ccccn3)cc2)c2cc(-c3ccc4c5c(cccc35)-c3ccccc3-4)cc(-c3ccc4c5c(cccc35)-c3ccccc3-4)c2)cc1 ; c1ccc(N(c2ccc(-c3cccnc3)cc2)c2cc(-c3ccc4c5c(cccc35)-c3ccccc3-4)cc(-c3ccc4c5c(cccc35)-c3ccccc3-4)c2)cc1 ; c1ccc(N(c2ccc(-c3ccncc3)cc2)c2cc(-c3ccc4c5c(cccc35)-c3ccccc3-4)cc(-c3ccc4c5c(cccc35)-c3ccccc3-4)c2)cc1]

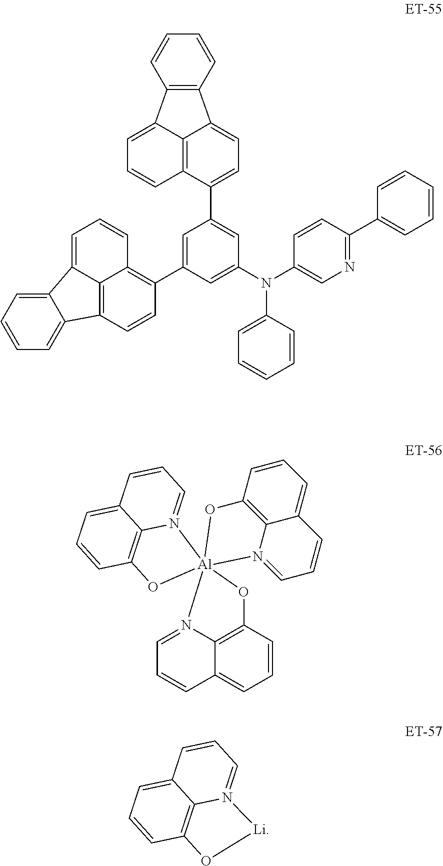

The device can also include an electron injection layer between the electron transporting layer and the cathode. The electron injection layer material includes, but is not limited to, one or a combination of more of LiQ, LiF, NaCl, CsF, Li₂O, Cs₂CO₃, BaO, Na, Li or Ca as listed below.

Compared with the prior art, the above mentioned solution of the disclosure has the following beneficial effects:

In the structure of compounds of the disclosure, the introduction of a substituent Ar² at the 1-position of naphthalene ring can not only adjust the adjacent steric hindrance size, but also effectively regulate the distortion degree of a molecule so as to reduce the crystallinity of the molecule. Secondly, the 2-position is substituted with an arylamino group, and the introduction of a trisubstituted structure on the arylamino group can effectively regulate the stereostructure of the molecule, improve the packing density of the molecule, so that the materials designed can meet the requirements of devices for materials.

In the disclosure, the naphthalene ring parent core structure substituted at the 1- and 2-position at the same time and coordinated with substituents such as Ar¹, Ar², R¹-R⁴, etc, can achieve the best effect, so that the material can improve the luminous efficiency, reduce the starting voltage and prolong the service life of the device when it is applied to the organic electroluminescent device, especially when it is used as an electron blocking layer.

In addition, the preparation process of the compound of the disclosure is simple and practicable, and the raw materials are ready available, which is suitable for large scale production.

When the brightness of the organic electroluminescent device containing the compound of formula I provided by the disclosure reaches 3000 cd/m², the driving voltage is as low as 3.8 V and below, and the current efficiency is as high as 18.2 cd/A and above.

In another preferred embodiments of the disclosure, an organic electroluminescent device with improved photoelectric performance is provided. The organic electroluminescent device includes an anode layer, a cathode layer and an organic layer arranged between the anode layer and the cathode layer;

The organic layer includes a light emitting layer, wherein the light emitting layer includes a host material and a doping material;

The host material includes a first host material and a second host material, the first host material has a structure as shown in Formula I;

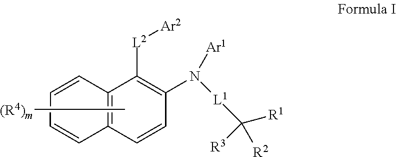

wherein, the Ar¹ and Ar² are independently selected from the group consisting of a substituted or unsubstituted C6-C30 aryl or a substituted or unsubstituted C3-C30 heteroaryl;

the L¹ is selected from the group consisting of a substituted or unsubstituted C6-C30 arylene or a substituted or unsubstituted C3-C30 heteroarylene;

the L² is selected from the group consisting of one of a single bond, a substituted or unsubstituted C6-C30 arylene or a substituted or unsubstituted C3-C30 heteroarylene;

the R¹, R² and R³ are independently selected from the group consisting of any one of a substituted or unsubstituted C1-C20 chain alkyl, a substituted or unsubstituted C3-C20 cycloalkyl, a substituted or unsubstituted C6-C30 arylamino, a substituted or unsubstituted C3-C30 heteroarylamino, a substituted or unsubstituted C6-C30 aryl, a substituted or unsubstituted C3-C30 heteroaryl;

the m is an integer from 0 to 6, for example, 1, 2, 3, 4, 5, etc.;

the R⁴ are independently selected from the group consisting of any one of a substituted or unsubstituted C1-C20 chain alkyl, a substituted or unsubstituted C3-C20 cycloalkyl, a substituted or unsubstituted C6-C30 arylamino, a substituted or unsubstituted C3-C30 heteroarylamino, a substituted or unsubstituted C6-C30 aryl;

in Ar¹, Ar², L¹, L², R¹, R², R³ and R⁴, the substituted or unsubstituted substituent is each independently selected from the group consisting of one or a combination of at least two of halogen, C1-C10 chain alkyl, C3-C10 cycloalkyl, C1-C10 alkoxy, C1-C10 thioalkoxy, C6-C30 arylamino, C3-C30 heteroarylamino, C6-C30 monocyclic aryl, C10-C30 fused ring aryl, C3-C30 monocyclic heteroaryl, and C6-C30 fused ring heteroaryl. The “substituted or unsubstituted substituent” refers to the selection range of the substituent when the “substituted or unsubstituted” group is a substituted group.

The aforementioned “substituted or unsubstituted” groups can be substituted with one substituent or multiple substituents. When the substituents are multiple, they can be selected from different substituents and have the same meaning when the disclosure involves the same expression, and the selection range of the substituents is as shown above and will not be repeated herein.

In the disclosure, the expression of chemical elements includes the concept of isotopes with the same chemical properties. For example, hydrogen (H) includes ¹H (protium or H), ²H (deuterium or D), etc; and carbon (C) includes ¹²C and ¹³C, etc.

In the disclosure, the heteroatom of a heteroaryl group usually refers to one selected from N, O, and S.

In the disclosure, the expression of the ring structure crossed by “—” indicates that the connection site is at any position on the ring structure where bonds can be formed.

The above-mentioned C1-C20 chain alkyl is preferably C1-C10 chain alkyl, more preferably C1-C6 chain alkyl, for example, methyl, ethyl, n-propyl, iso-propyl, n-butyl, iso-butyl, tert-butyl, n-hexyl, n-octyl, n-pentyl, n-heptyl, n-nonyl, n-decyl, etc., can be listed.

The above-mentioned C3-C20 cycloalkyl is preferably cyclopropyl, cyclobutyl, cyclopentyl, and cyclohexyl.

The above mentioned substituted or unsubstituted C6-C30 aryl(ene), preferably C6-C20 aryl(ene) and preferably the aryl is a group from the group consisting of phenyl, biphenyl, terphenyl, naphthyl, anthracyl, phenanthryl, indenyl, fluorenyland derivatives thereof, fluoranthenyl, triphenylene, pyrenyl, perylene, chrysenyl and tetracenyl. The biphenyl is selected from the group consisting of 2-biphenyl, 3-biphenyl and 4-biphenyl; the terphenyl includes para-terphenyl-4-yl, para-terphenyl-3-yl, para-terphenyl-2-yl, meta-terphenyl-4-yl, meta-terphenyl-3-yl and meta-terphenyl-2-yl; the naphthyl includes 1-naphthyl or 2-naphthyl; the anthracyl is selected from the group consisting of 1-anthracyl, 2-anthracyl and 9-anthracyl; the fluorenyl is selected from the group consisting of 1-fluorenyl, 2-fluorenyl, 3-fluorenyl, 4-fluorenyl and 9-fluorenyl; the fluorenyl derivative is selected from the group consisting of 9,9-dimethylfluorene, 9,9-spirobifluorene and benzofluorene; the pyrenyl is selected from the group consisting of 1-pyrenyl, 2-pyrenyl and 4-pyrenyl; and the tetracenyl is selected from the group consisting of 1-tetracenyl, 2-tetracenyl and 9-tetracenyl.

In the disclosure, the aryl includes monocyclic aryl and fused ring aryl, the heteroaryl also includes monocyclic heteroaryl and fused ring heteroaryl.

The above mentioned substituted or unsubstituted C3-C30 heteroaryl(ene), preferably C4-C20 heteroaryl(ene), preferably the heteroaryl is furanyl, thienyl, pyrrolyl, benzofuranyl, benzothienyl, isobenzofuranyl, indolyl, dibenzofuranyl, dibenzothiophenyl, carbazolyland derivatives thereof, wherein, the carbazolyl derivative preferably is 9-phenylcarbazole, 9-naphthylcarbazolebenzocarbazole, dibenzocarbazole, or indolocarbazole.

The above mentioned C6-C30 arylamino refers to a group formed by connecting aryl to amino, the connecting bond can be on the amino group or on the aryl group. The C3-C30 heteroaryl amino group is in a similar way.

The above preferred embodiments provide an organic electroluminescent device using a dual host light emitting layer, in which the first host material selects the compound as shown in Formula I, which has a high hole mobility and a suitable energy level, and can adjust the carrier distribution inside the light emitting layer, so as to regulate the carrier composite region, and has a high spatial packing structure, when it is used as one of the dual hosts, it is combined with the second host material to precisely control the distribution of carriers inside the light emitting layer, so as to improve the light extraction efficiency of the organic electroluminescent devices, and thus improving the photoelectric performance of the devices.

Preferably, In Formula I, the Ar¹ is selected from the group consisting of any of the following substituted or unsubstituted groups: phenyl, biphenyl, terphenyl, naphthyl, phenanthryl, anthracyl, dibenzofuranyl, dibenzothiophenyl, carbazolyl, 9,9dimethylfluorenyl, 9,9diphenylfluorenyl, spirofluorenyl, triphenylene, fluoranthenyl, benzo9,9dimethylfluorenyl, and benzospirofluorenyl.

And/or, In Formula I, the Ar² is selected from the group consisting of any of the following substituted or unsubstituted groups: phenyl, biphenyl, terphenyl, naphthyl, phenanthryl, anthracyl, dibenzofuranyl, dibenzothiophenyl, carbazolyl, 9,9dimethylfluorenyl, 9,9diphenylfluorenyl, spirofluorenyl, triphenylene, fluoranthenyl, benzo9,9dimethylfluorenyl, and benzospirofluorenyl, preferably a substituted or unsubstituted naphthyl.

Preferably, the Ar² is selected from the group consisting of any of the following substituted or unsubstituted groups:

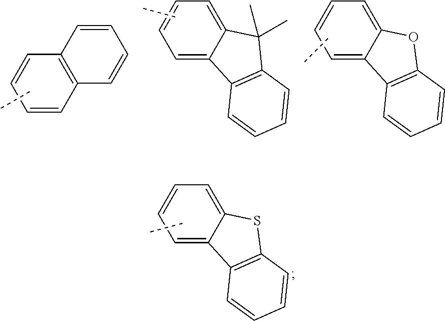

wherein, the dotted line represents a connecting bond of the groups.

Preferably, In Formula I, the R¹, R² and R³ are independently selected from the group consisting of one of methyl, ethyl or phenyl, preferably are all methyl.

Preferably, In Formula I, the L¹ is selected from the group consisting of one of the following substituted or unsubstituted groups: phenylene, biphenylene, naphthylene, dibenzofuranylene, dibenzothiophenylene, and 9,9dimethylfluorenylene.

Preferably, In Formula I, the L² is selected from the group consisting of one of a single bond, a substituted or unsubstituted C6-C20 arylene or a substituted or unsubstituted C3-C20 heteroarylene, preferably a single bond or phenylene.

Preferably, the first host material is selected from the group consisting of any one or a combination of at least two of the aforementioned compound P1 to compound P777.

Preferably, the mass ratio of the first host material to the second host material is 0.01:1-1.5:1, for example, 0.05:1, 0.1:1, 0.2:1, 1:0.3, 0.4:1, 0.5:1, 0.6:1, 0.7:1, 0.8:1, 0.9:1, 1:1.0, 1.1:1, 1.2:1, 1.3:1, 1.4:1, etc., preferably 0.1:1-1:1.

Preferably, in the disclosure, the mass ratio of the first host material (i.e. compound of Formula I) to the second host material is 0.01:1-1.5:1, within the aforementioned range, the photoelectric performance of the device is optimal. If the addition amount of compound of Formula I is too much, the voltage of the device will increase and the device efficiency will decrease, while if the addition amount is too little, the device efficiency will not increase significantly.

Preferably, the HOMO energy level of the second host material is −5.3 eV to −5.7 eV, for example, −5.4 eV, −5.5 eV, −5.6 eV, etc.:

and/or, the LUMO energy level of the second host material is −2.3 eV to −2.6 eV, for example, −2.4 eV, −2.5 eV etc.

Preferably, the HOMO energy level of the second host material is −5.3 eV to −5.7 eV, and the LUMO energy level is −2.3 eV to −2.6 eV.

Preferably, the second host material of the disclosure has the aforementioned specific HOMO energy level and LUMO energy level, so that it can be better matched with the first host material and more accurately regulate the distribution of carriers inside the light emitting layer, thereby further improving the light extraction efficiency of the organic electroluminescent device and improving the device efficiency.

Preferably, the second host material is selected from the group consisting of any one or a combination of at least two of the following compound PH-1 to compound PH-85:

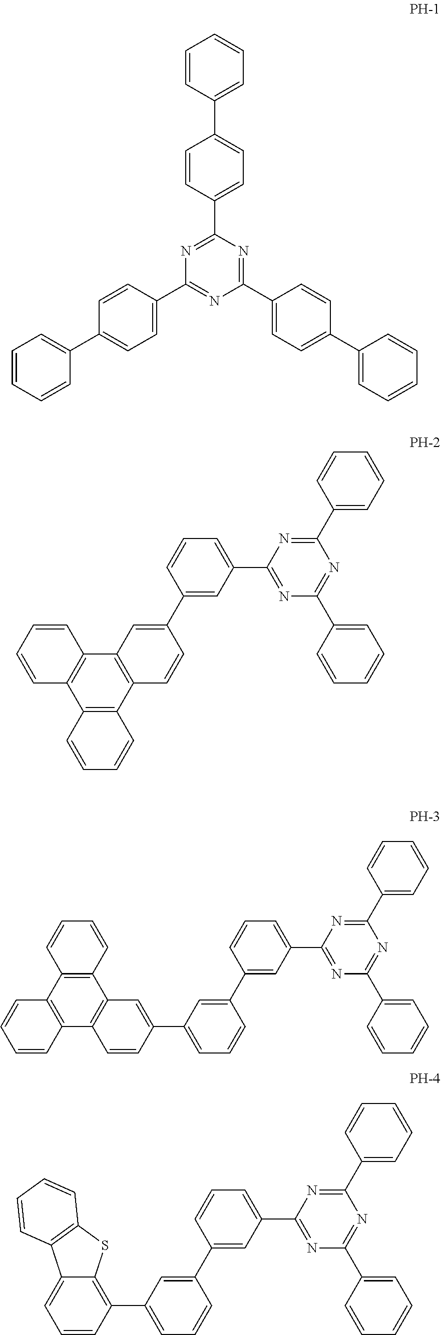 [SMILES: c1ccc(-c2ccc(-c3nc(-c4ccc(-c5ccccc5)cc4)nc(-c4ccc(-c5ccccc5)cc4)n3)cc2)cc1 ; c1ccc(-c2nc(-c3ccccc3)nc(-c3cccc(-c4ccc5c6ccccc6c6ccccc6c5c4)c3)n2)cc1 ; c1ccc(-c2nc(-c3ccccc3)nc(-c3cccc(-c4cccc(-c5ccc6c7ccccc7c7ccccc7c6c5)c4)c3)n2)cc1 ; c1ccc(-c2nc(-c3ccccc3)nc(-c3cccc(-c4cccc(-c5cccc6c5sc5ccccc56)c4)c3)n2)cc1]

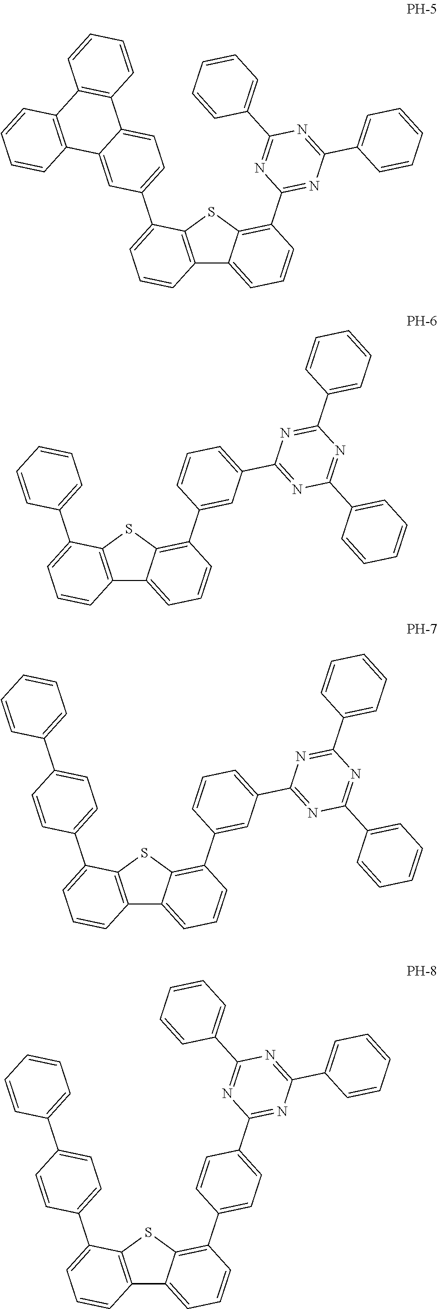 [SMILES: c1ccc(-c2nc(-c3ccccc3)nc(-c3cccc4c3sc3c(-c5ccc6c7ccccc7c7ccccc7c6c5)cccc34)n2)cc1 ; c1ccc(-c2nc(-c3ccccc3)nc(-c3cccc(-c4cccc5c4sc4c(-c6ccccc6)cccc45)c3)n2)cc1 ; c1ccc(-c2ccc(-c3cccc4c3sc3c(-c5cccc(-c6nc(-c7ccccc7)nc(-c7ccccc7)n6)c5)cccc34)cc2)cc1 ; c1ccc(-c2ccc(-c3cccc4c3sc3c(-c5ccc(-c6nc(-c7ccccc7)nc(-c7ccccc7)n6)cc5)cccc34)cc2)cc1]

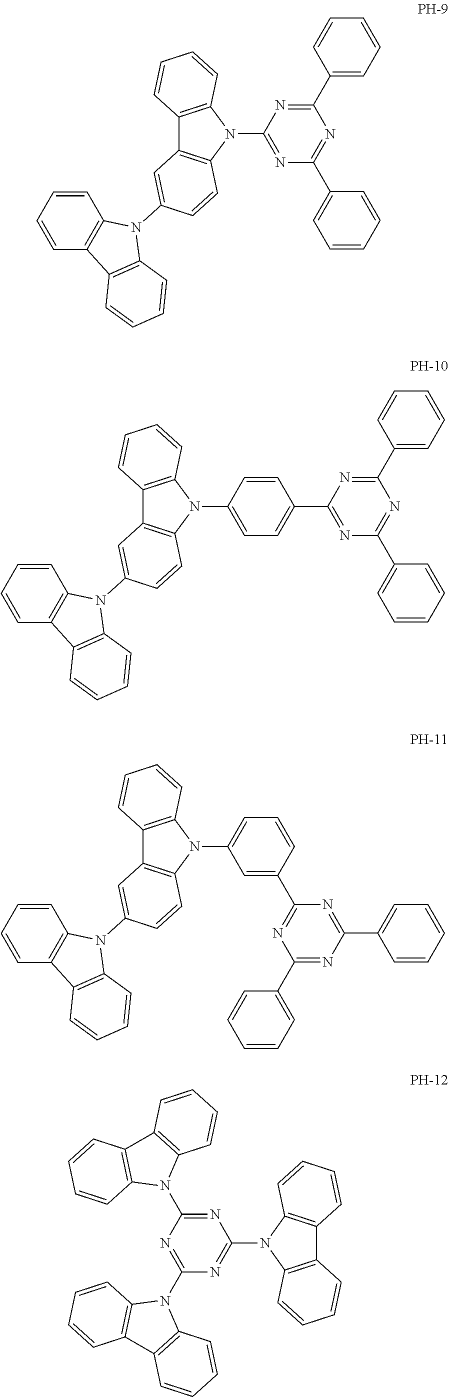 [SMILES: c1ccc(-c2nc(-c3ccccc3)nc(-n3c4ccccc4c4cc(-n5c6ccccc6c6ccccc65)ccc43)n2)cc1 ; c1ccc(-c2nc(-c3ccccc3)nc(-c3ccc(-n4c5ccccc5c5cc(-n6c7ccccc7c7ccccc76)ccc54)cc3)n2)cc1 ; c1ccc(-c2nc(-c3ccccc3)nc(-c3cccc(-n4c5ccccc5c5cc(-n6c7ccccc7c7ccccc76)ccc54)c3)n2)cc1 ; c1ccc2c(c1)c1ccccc1n2-c1nc(-n2c3ccccc3c3ccccc32)nc(-n2c3ccccc3c3ccccc32)n1]

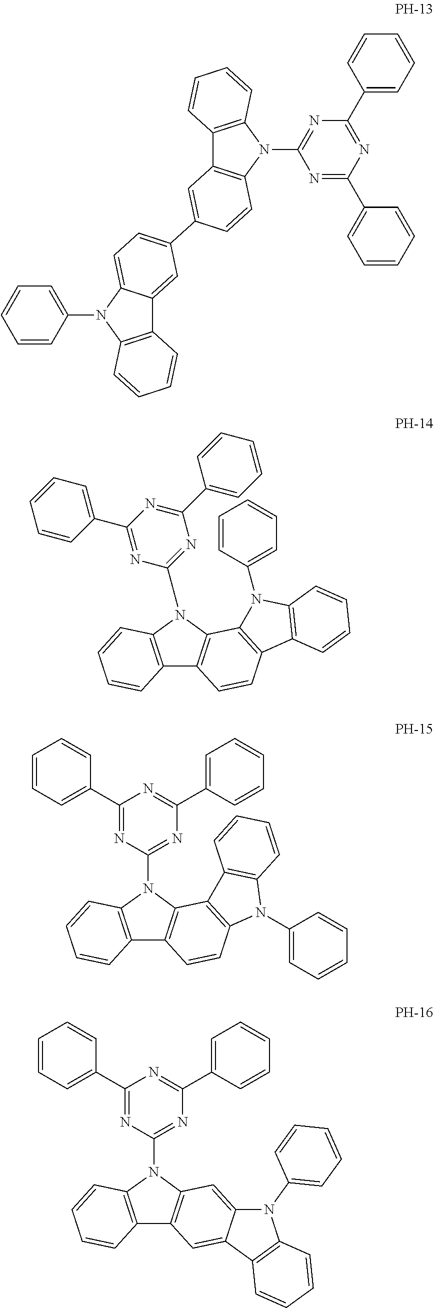 [SMILES: c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2nc(-c3ccccc3)nc(-n3c4ccccc4c4ccc5c6ccccc6n(-c6ccccc6)c5c43)n2)cc1 ; c1ccc(-c2nc(-c3ccccc3)nc(-n3c4ccccc4c4ccc5c(c6ccccc6n5-c5ccccc5)c43)n2)cc1 ; c1ccc(-c2nc(-c3ccccc3)nc(-n3c4ccccc4c4cc5c6ccccc6n(-c6ccccc6)c5cc43)n2)cc1]

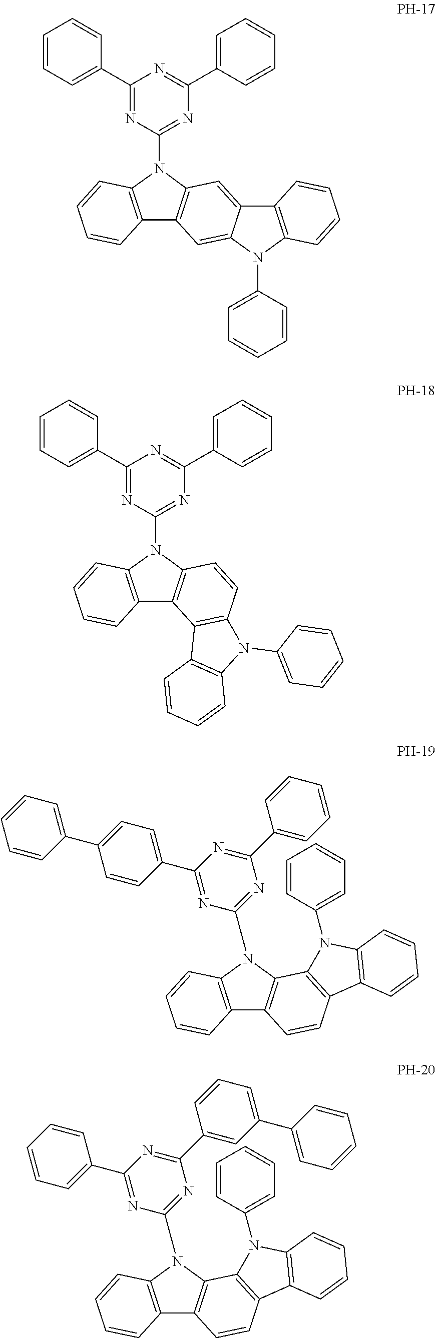

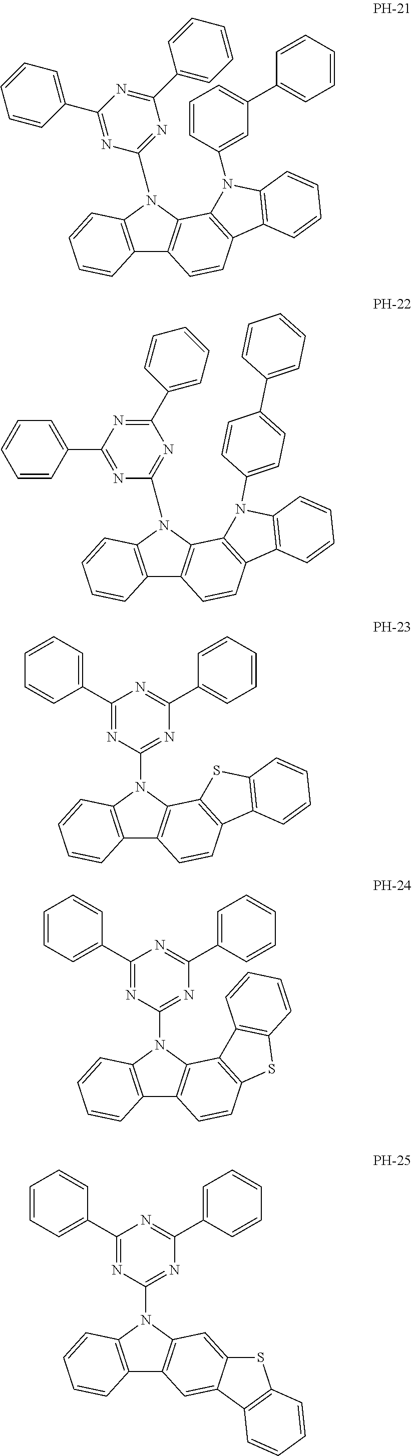

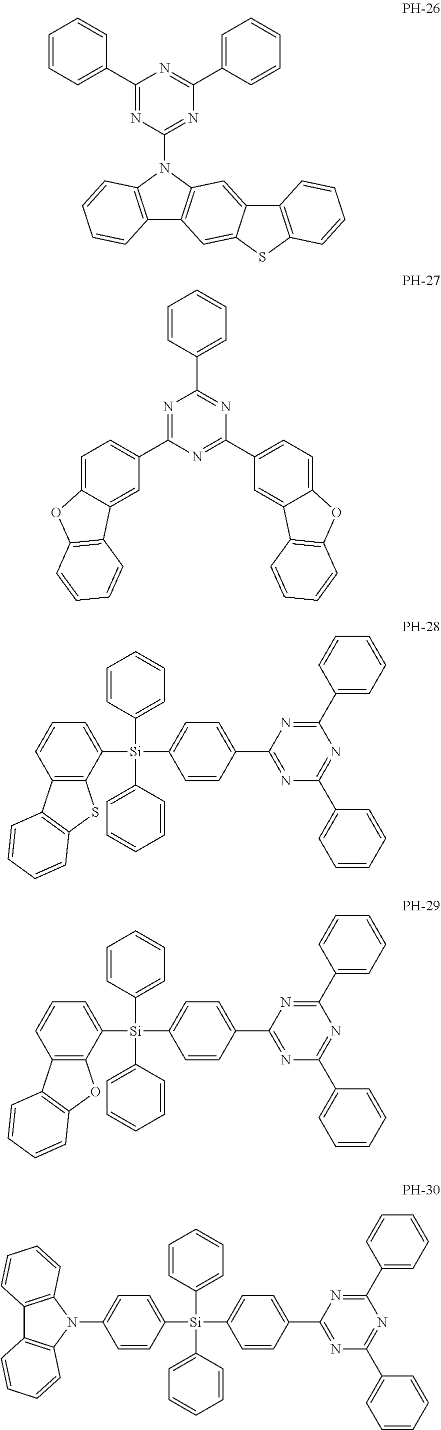

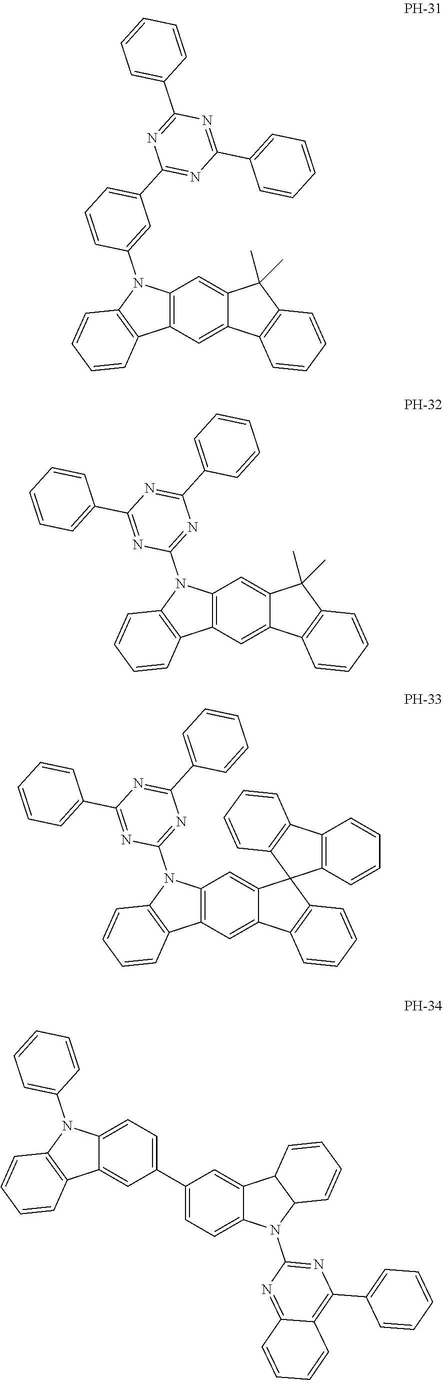 [SMILES: CC1(C)c2ccccc2-c2cc3c4ccccc4n(-c4cccc(-c5nc(-c6ccccc6)nc(-c6ccccc6)n5)c4)c3cc21 ; CC1(C)c2ccccc2-c2cc3c4ccccc4n(-c4nc(-c5ccccc5)nc(-c5ccccc5)n4)c3cc21 ; c1ccc(-c2nc(-c3ccccc3)nc(-n3c4ccccc4c4cc5c(cc43)C3(c4ccccc4-c4ccccc43)c3ccccc3-5)n2)cc1 ; C1=CC2c3cc(-c4ccc5c(c4)c4ccccc4n5-c4ccccc4)ccc3N(c3nc(-c4ccccc4)c4ccccc4n3)C2C=C1]

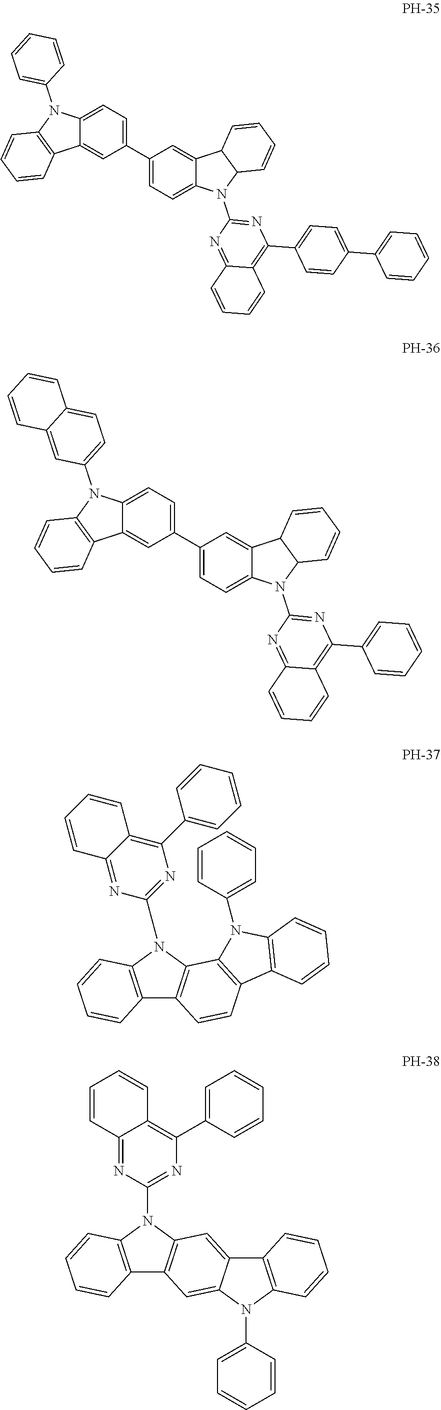 [SMILES: C1=CC2c3cc(-c4ccc5c(c4)c4ccccc4n5-c4ccccc4)ccc3N(c3nc(-c4ccc(-c5ccccc5)cc4)c4ccccc4n3)C2C=C1 ; C1=CC2c3cc(-c4ccc5c(c4)c4ccccc4n5-c4ccc5ccccc5c4)ccc3N(c3nc(-c4ccccc4)c4ccccc4n3)C2C=C1 ; c1ccc(-c2nc(-n3c4ccccc4c4ccc5c6ccccc6n(-c6ccccc6)c5c43)nc3ccccc23)cc1 ; c1ccc(-c2nc(-n3c4ccccc4c4cc5c(cc43)c3ccccc3n5-c3ccccc3)nc3ccccc23)cc1]

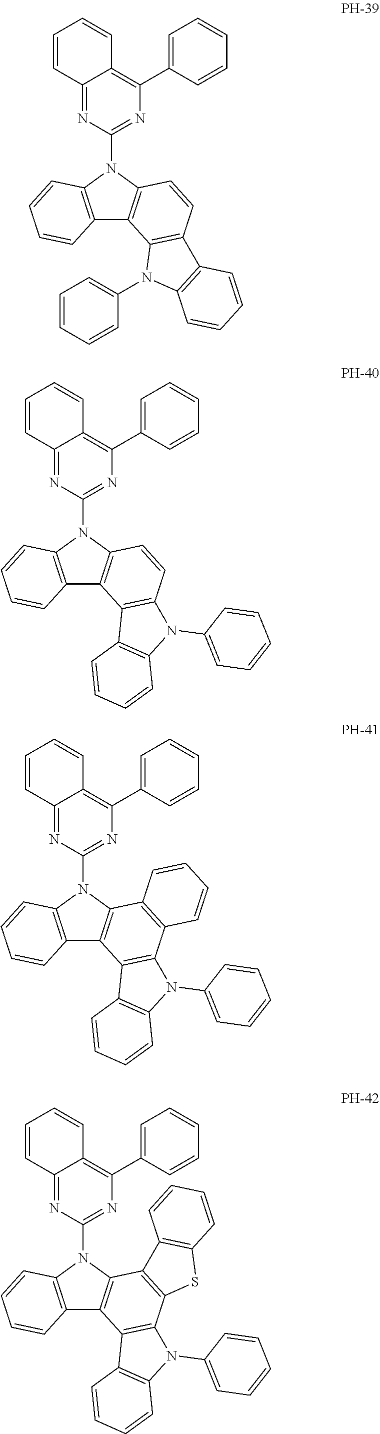 [SMILES: c1ccc(-c2nc(-n3c4ccccc4c4c3ccc3c5ccccc5n(-c5ccccc5)c34)nc3ccccc23)cc1 ; c1ccc(-c2nc(-n3c4ccccc4c4c5c6ccccc6n(-c6ccccc6)c5ccc43)nc3ccccc23)cc1 ; c1ccc(-c2nc(-n3c4ccccc4c4c5c6ccccc6n(-c6ccccc6)c5c5ccccc5c43)nc3ccccc23)cc1 ; c1ccc(-c2nc(-n3c4ccccc4c4c5c6ccccc6n(-c6ccccc6)c5c5sc6ccccc6c5c43)nc3ccccc23)cc1]

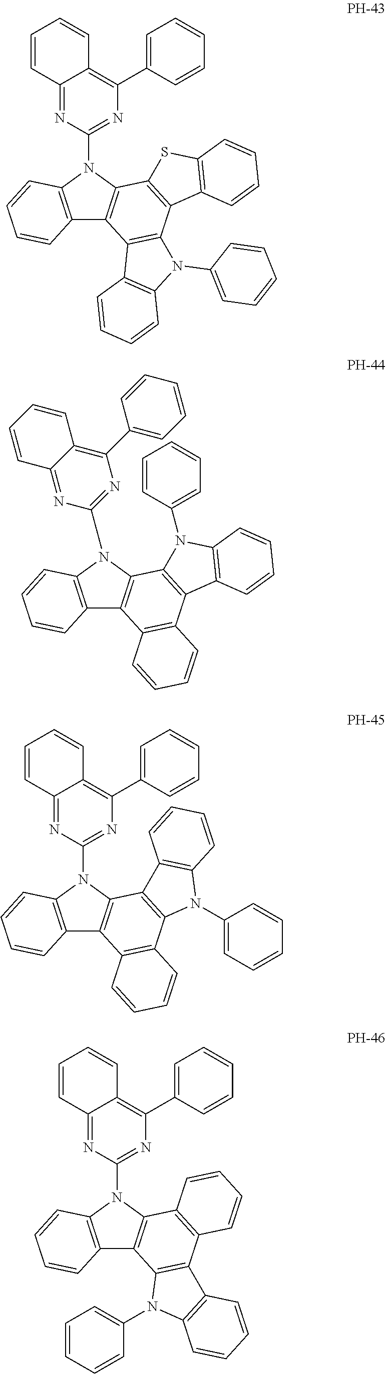

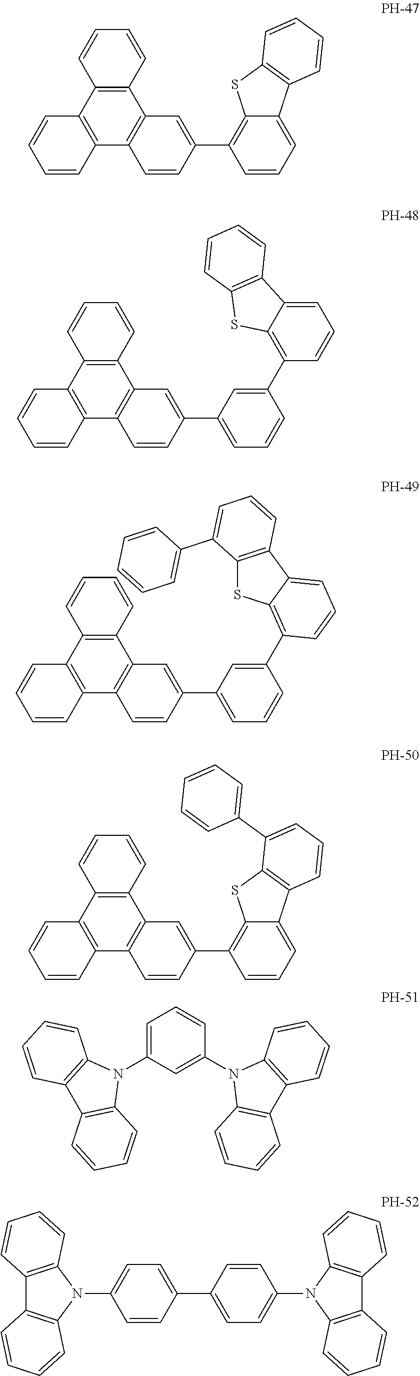

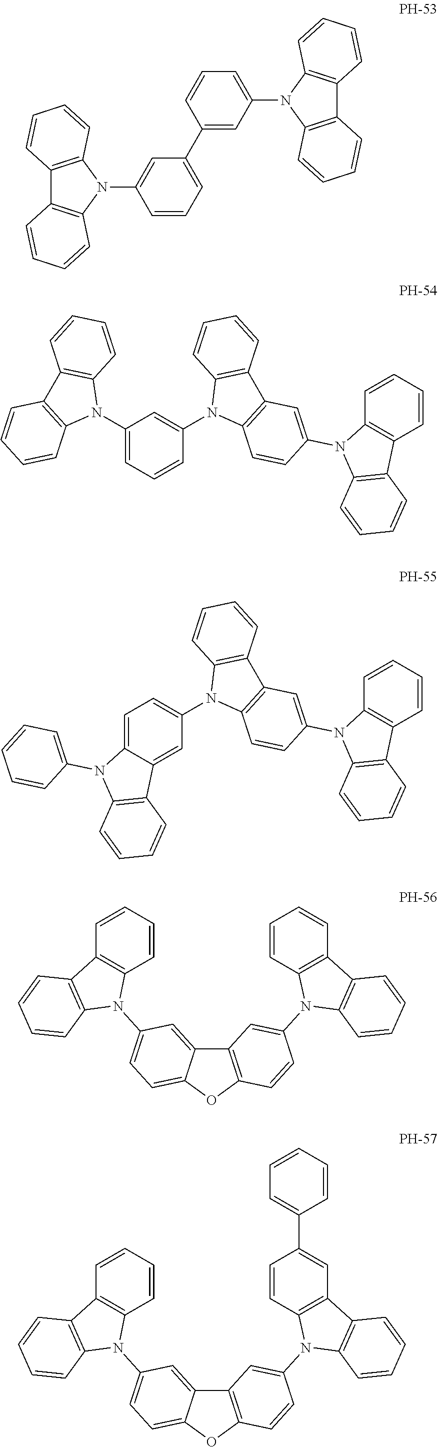 [SMILES: c1cc(-c2cccc(-n3c4ccccc4c4ccccc43)c2)cc(-n2c3ccccc3c3ccccc32)c1 ; c1cc(-n2c3ccccc3c3ccccc32)cc(-n2c3ccccc3c3cc(-n4c5ccccc5c5ccccc54)ccc32)c1 ; c1ccc(-n2c3ccccc3c3cc(-n4c5ccccc5c5cc(-n6c7ccccc7c7ccccc76)ccc54)ccc32)cc1 ; c1ccc2c(c1)c1ccccc1n2-c1ccc2oc3ccc(-n4c5ccccc5c5ccccc54)cc3c2c1 ; c1ccc(-c2ccc3c(c2)c2ccccc2n3-c2ccc3oc4ccc(-n5c6ccccc6c6ccccc65)cc4c3c2)cc1]

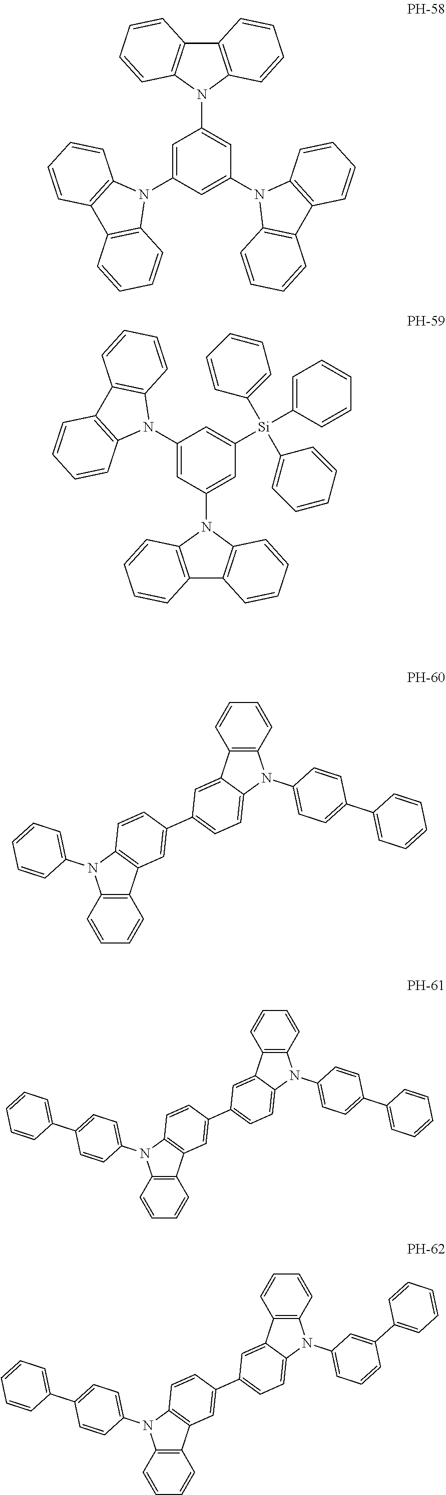 [SMILES: c1ccc2c(c1)c1ccccc1n2-c1cc(-n2c3ccccc3c3ccccc32)cc(-n2c3ccccc3c3ccccc32)c1 ; c1ccc([Si](c2ccccc2)(c2ccccc2)c2cc(-n3c4ccccc4c4ccccc43)cc(-n3c4ccccc4c4ccccc43)c2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-n3c4ccccc4c4cc(-c5ccc6c(c5)c5ccccc5n6-c5cccc(-c6ccccc6)c5)ccc43)cc2)cc1]

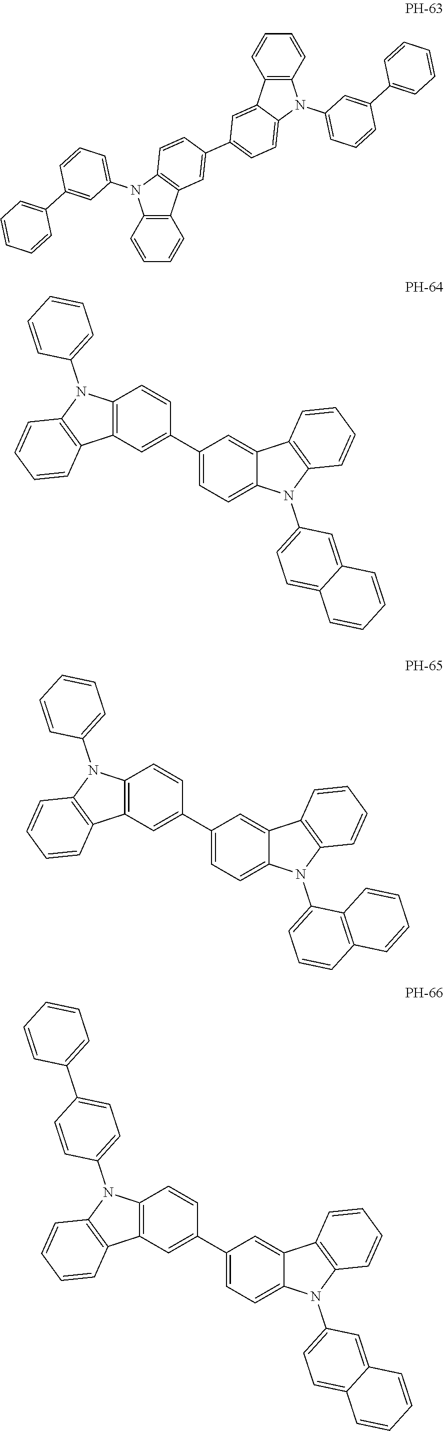 [SMILES: c1ccc(-c2cccc(-n3c4ccccc4c4cc(-c5ccc6c(c5)c5ccccc5n6-c5cccc(-c6ccccc6)c5)ccc43)c2)cc1 ; c1ccc(-n2c3ccccc3c3cc(-c4ccc5c(c4)c4ccccc4n5-c4ccc5ccccc5c4)ccc32)cc1 ; c1ccc(-n2c3ccccc3c3cc(-c4ccc5c(c4)c4ccccc4n5-c4cccc5ccccc45)ccc32)cc1 ; c1ccc(-c2ccc(-n3c4ccccc4c4cc(-c5ccc6c(c5)c5ccccc5n6-c5ccc6ccccc6c5)ccc43)cc2)cc1]

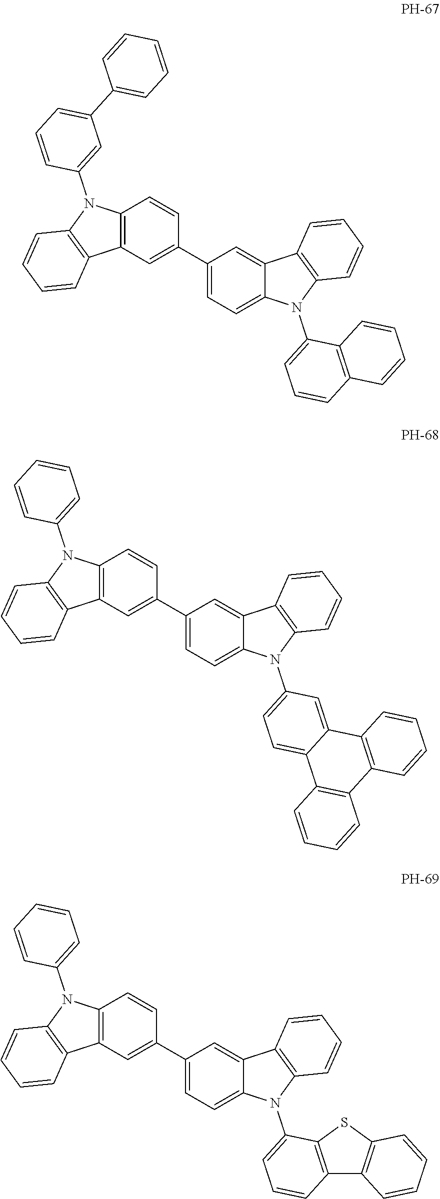 [SMILES: c1ccc(-c2cccc(-n3c4ccccc4c4cc(-c5ccc6c(c5)c5ccccc5n6-c5cccc6ccccc56)ccc43)c2)cc1 ; c1ccc(-n2c3ccccc3c3cc(-c4ccc5c(c4)c4ccccc4n5-c4ccc5c6ccccc6c6ccccc6c5c4)ccc32)cc1 ; c1ccc(-n2c3ccccc3c3cc(-c4ccc5c(c4)c4ccccc4n5-c4cccc5c4sc4ccccc45)ccc32)cc1]

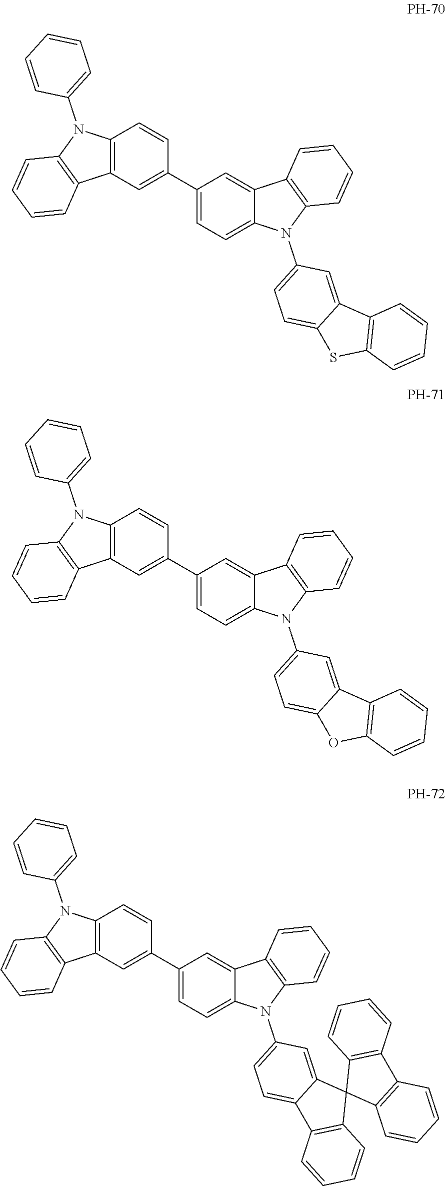 [SMILES: c1ccc(-n2c3ccccc3c3cc(-c4ccc5c(c4)c4ccccc4n5-c4ccc5sc6ccccc6c5c4)ccc32)cc1 ; c1ccc(-n2c3ccccc3c3cc(-c4ccc5c(c4)c4ccccc4n5-c4ccc5oc6ccccc6c5c4)ccc32)cc1 ; c1ccc(-n2c3ccccc3c3cc(-c4ccc5c(c4)c4ccccc4n5-c4ccc5c(c4)C4(c6ccccc6-c6ccccc64)c4ccccc4-5)ccc32)cc1]

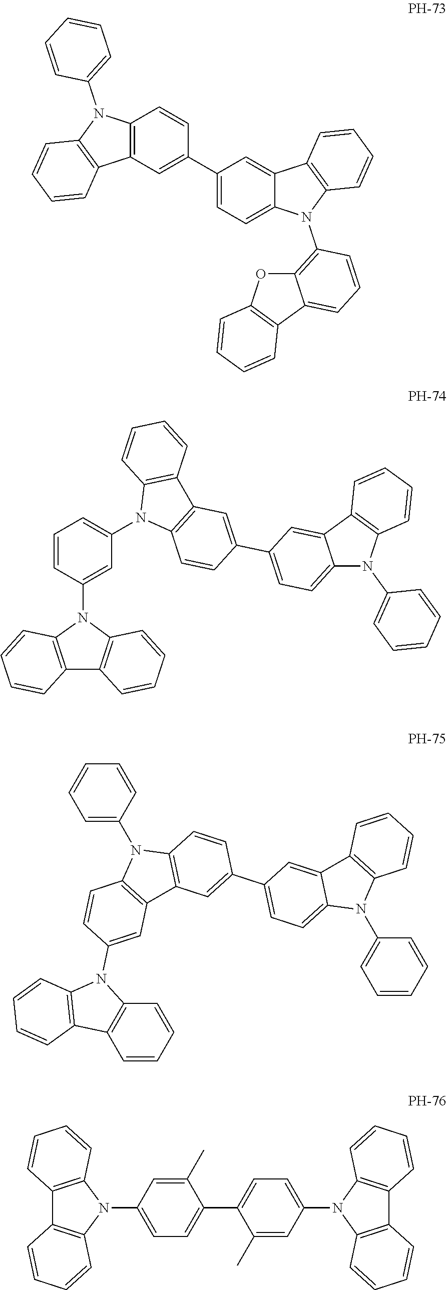 [SMILES: c1ccc(-n2c3ccccc3c3cc(-c4ccc5c(c4)c4ccccc4n5-c4cccc5c4oc4ccccc45)ccc32)cc1 ; c1ccc(-n2c3ccccc3c3cc(-c4ccc5c(c4)c4ccccc4n5-c4cccc(-n5c6ccccc6c6ccccc65)c4)ccc32)cc1 ; c1ccc(-n2c3ccccc3c3cc(-c4ccc5c(c4)c4cc(-n6c7ccccc7c7ccccc76)ccc4n5-c4ccccc4)ccc32)cc1 ; Cc1cc(-n2c3ccccc3c3ccccc32)ccc1-c1ccc(-n2c3ccccc3c3ccccc32)cc1C]

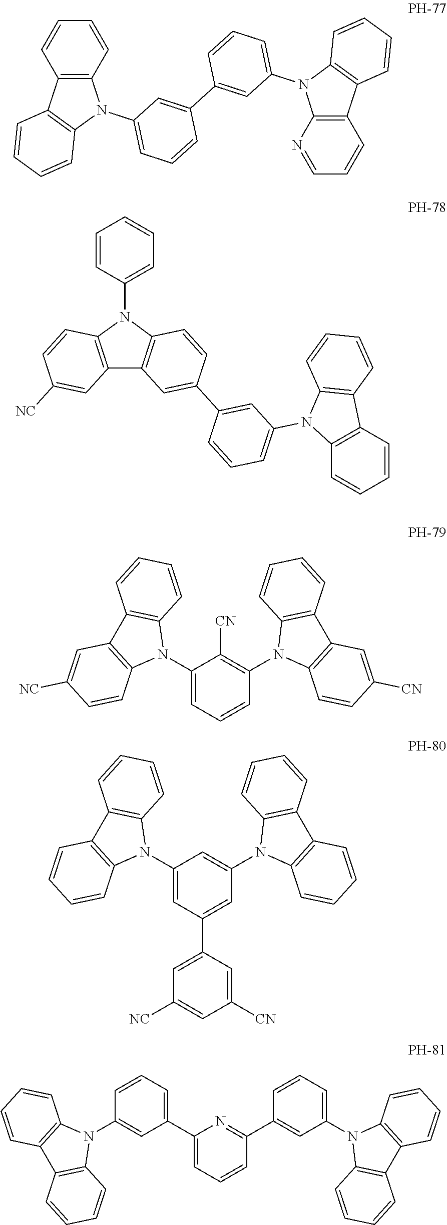 [SMILES: c1cc(-c2cccc(-n3c4ccccc4c4cccnc43)c2)cc(-n2c3ccccc3c3ccccc32)c1 ; N#Cc1ccc2c(c1)c1cc(-c3cccc(-n4c5ccccc5c5ccccc54)c3)ccc1n2-c1ccccc1 ; N#Cc1ccc2c(c1)c1ccccc1n2-c1cccc(-n2c3ccccc3c3cc(C#N)ccc32)c1C#N ; N#Cc1cc(C#N)cc(-c2cc(-n3c4ccccc4c4ccccc43)cc(-n3c4ccccc4c4ccccc43)c2)c1 ; c1cc(-c2cccc(-c3cccc(-n4c5ccccc5c5ccccc54)c3)n2)cc(-n2c3ccccc3c3ccccc32)c1]

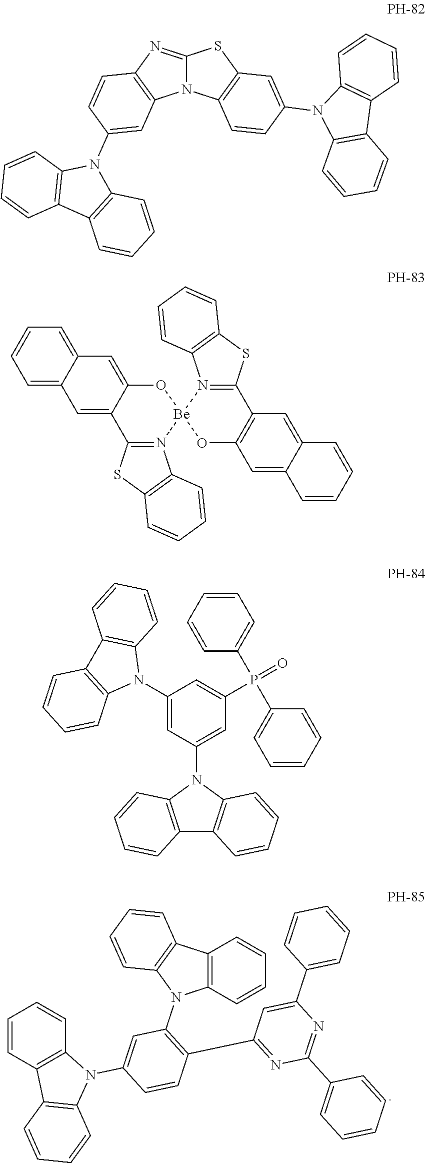

Preferably, the thickness of the light emitting layer is 10-65 nm, for example, 15 nm, 20 nm, 25 nm, 30 nm, 35 nm, 40 nm, 45 nm, 50 nm, 55 nm, 60 nm, etc., preferably 15-55 nm.

Based on the specific material composition of the light emitting layer in the above preferred embodiments, preferably the thickness of the light emitting layer in the disclosure is 10-65 nm. Within this thickness range, the efficiency of the device is further improved. If the thickness is too low, it will lead to the shift of the chromaticity of the device and decrease of efficiency, and if the thickness is too high, it will lead to the increase of the device voltage and the decrease of efficiency.

In the disclosure, the first host material and the second host material can be co-evaporated or pre-mixed to obtain a light emitting layer, but not limited to co-evaporation or pre-mixing.

Preferably, the organic layer also includes any one or a combination of at least two of a hole injection layer, a hole transporting layer, an electron blocking layer, an electron transporting layer or an electron injection layer.

The organic layer in the OLED can be divided into multiple regions. For example, the organic material layer may include a hole transporting region, a light emitting layer, and an electron transporting region.

In specific embodiments, a substrate can be used below the first electrode or above the second electrode. The substrate is glass or a polymer material with excellent mechanical strength, thermal stability water resistance and transparency. In addition, the substrate used as the display can also be equipped with thin film transistors (TFTs).

The first electrode can be formed by sputtering or depositing a material used as the first electrode on the substrate. When the first electrode is used as the anode, an oxide transparent conductive material such as indium tin oxide (ITO), indium zinc oxide (IZO), tin dioxide (SnO₂), zinc oxide (ZnO), etc., and any combination thereof can be used. When the first electrode is used as the cathode, a metal or alloy such as magnesium (Mg), silver (Ag), aluminum (Al), aluminum-lithium (Al—Li), calcium (Ca), ytterbium (Yb) magnesium-indium (Mg—In), magnesium-silver (Mg—Ag), etc., as well as any combination thereof can be used.

The organic material layer can be formed on the electrode by methods such as vacuum thermal evaporation, rotary coating, and printing, etc. The compounds used as the organic material layer can be an organic small molecule, an organic macromolecule and a polymer, and a combination thereof.

The hole transporting region is located between the anode and the light emitting layer. The hole transporting region can be a hole transporting layer (HTL) with a single-layer structure, including a single-layer hole transporting layer containing only one compound and a single-layer hole transporting layer containing multiple compounds. The hole transporting region can be a multilayer structure including at least one of a hole injection layer (HIL), a hole transporting layer (HTL) and an electron blocking layer (EBL), wherein HIL is located between anode and HTL, and EBL is located between HTL and light emitting layer.

The materials of the hole transporting region can be selected from the group consisting of, but not limited to a phthalocyanine derivative such as CuPc, a conductive polymer or a polymer containing a conductive doping material such as polyphenylenevinylene, polyaniline/dodecyl benzene sulfonic acid (Pani/DBSA), poly(3,4-ethylenedioxythiophene)/poly(4-styrenesulfonate) (PEDOT/PSS), polyaniline/camphor sulfonic acid (Pani/CSA), polyaniline/poly(4-styrenesulfonate) (Pani/PSS), an aromatic amine derivative as shown in HT-1 to HT-51 below (wherein HT-1 to HT-34 are as described above, and the structures of HT-35 to HT-51 are as follows); or any combination thereof.

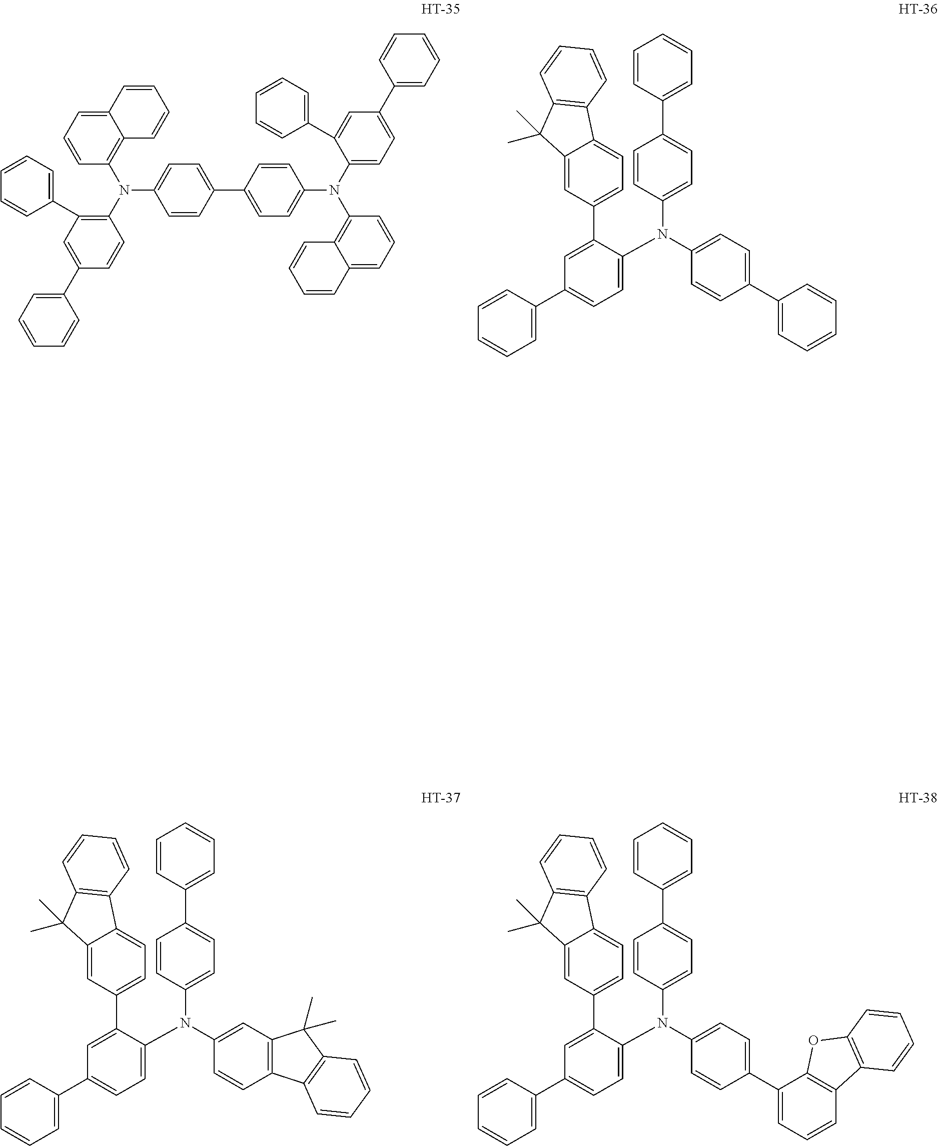 [SMILES: c1ccc(-c2ccc(N(c3ccc(-c4ccc(N(c5ccc(-c6ccccc6)cc5-c5ccccc5)c5cccc6ccccc56)cc4)cc3)c3cccc4ccccc34)c(-c3ccccc3)c2)cc1 ; CC1(C)c2ccccc2-c2ccc(-c3cc(-c4ccccc4)ccc3N(c3ccc(-c4ccccc4)cc3)c3ccc(-c4ccccc4)cc3)cc21 ; CC1(C)c2ccccc2-c2ccc(-c3cc(-c4ccccc4)ccc3N(c3ccc(-c4ccccc4)cc3)c3ccc4c(c3)C(C)(C)c3ccccc3-4)cc21 ; CC1(C)c2ccccc2-c2ccc(-c3cc(-c4ccccc4)ccc3N(c3ccc(-c4ccccc4)cc3)c3ccc(-c4cccc5c4oc4ccccc45)cc3)cc21]

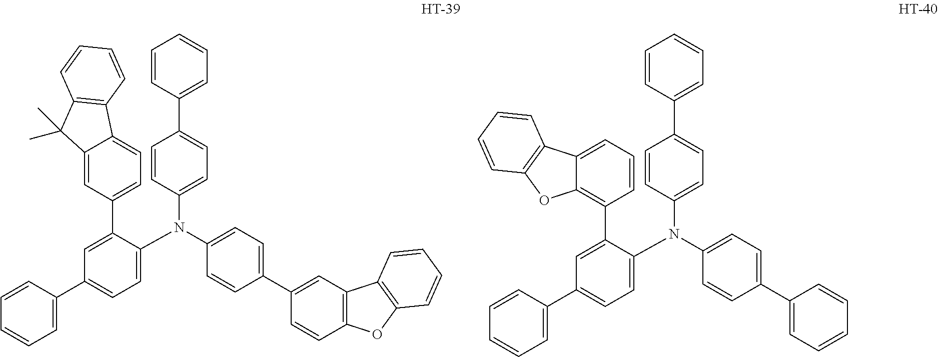 [SMILES: CC1(C)c2ccccc2-c2ccc(-c3cc(-c4ccccc4)ccc3N(c3ccc(-c4ccccc4)cc3)c3ccc(-c4ccc5oc6ccccc6c5c4)cc3)cc21 ; c1ccc(-c2ccc(N(c3ccc(-c4ccccc4)cc3)c3ccc(-c4ccccc4)cc3-c3cccc4c3oc3ccccc34)cc2)cc1]

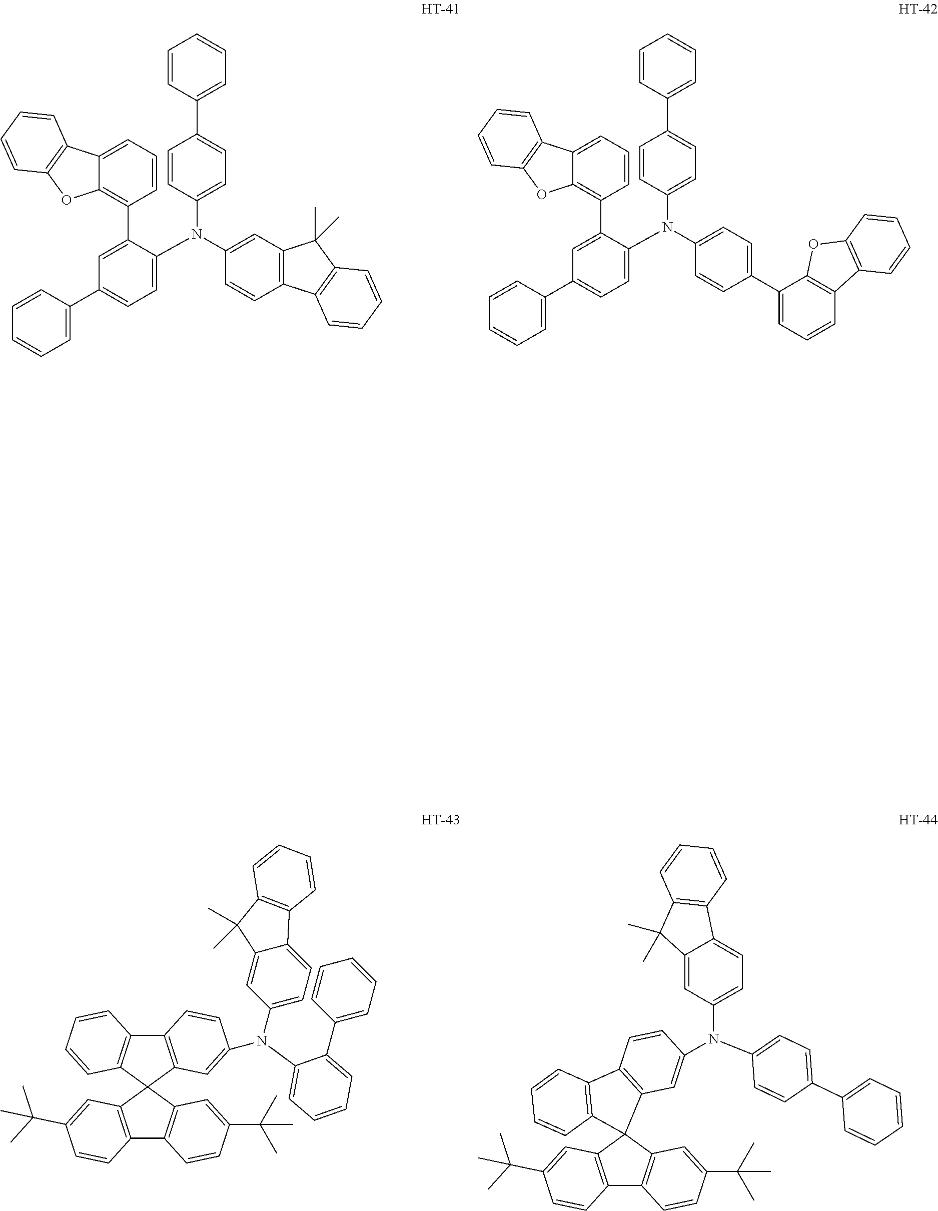 [SMILES: CC1(C)c2ccccc2-c2ccc(N(c3ccc(-c4ccccc4)cc3)c3ccc(-c4ccccc4)cc3-c3cccc4c3oc3ccccc34)cc21 ; c1ccc(-c2ccc(N(c3ccc(-c4cccc5c4oc4ccccc45)cc3)c3ccc(-c4ccccc4)cc3-c3cccc4c3oc3ccccc34)cc2)cc1 ; CC(C)(C)c1ccc2c(c1)C1(c3ccccc3-c3ccc(N(c4ccc5c(c4)C(C)(C)c4ccccc4-5)c4ccccc4-c4ccccc4)cc31)c1cc(C(C)(C)C)ccc1-2 ; CC(C)(C)c1ccc2c(c1)C1(c3ccccc3-c3ccc(N(c4ccc(-c5ccccc5)cc4)c4ccc5c(c4)C(C)(C)c4ccccc4-5)cc31)c1cc(C(C)(C)C)ccc1-2]

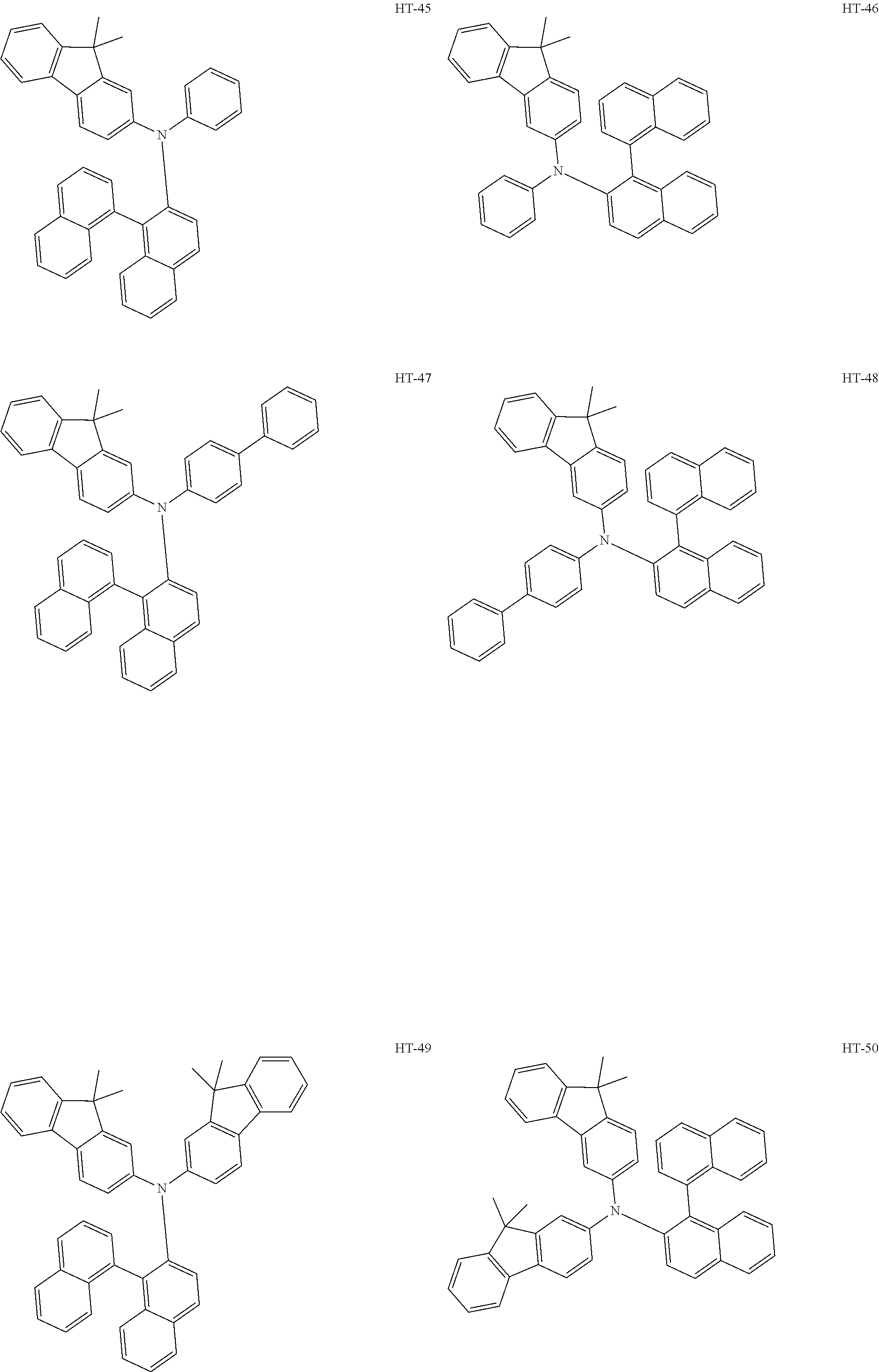 [SMILES: CC1(C)c2ccccc2-c2ccc(N(c3ccccc3)c3ccc4ccccc4c3-c3cccc4ccccc34)cc21 ; CC1(C)c2ccccc2-c2cc(N(c3ccccc3)c3ccc4ccccc4c3-c3cccc4ccccc34)ccc21 ; CC1(C)c2ccccc2-c2ccc(N(c3ccc(-c4ccccc4)cc3)c3ccc4ccccc4c3-c3cccc4ccccc34)cc21 ; CC1(C)c2ccccc2-c2cc(N(c3ccc(-c4ccccc4)cc3)c3ccc4ccccc4c3-c3cccc4ccccc34)ccc21 ; CC1(C)c2ccccc2-c2ccc(N(c3ccc4c(c3)C(C)(C)c3ccccc3-4)c3ccc4ccccc4c3-c3cccc4ccccc34)cc21 ; CC1(C)c2ccccc2-c2cc(N(c3ccc4c(c3)C(C)(C)c3ccccc3-4)c3ccc4ccccc4c3-c3cccc4ccccc34)ccc21]

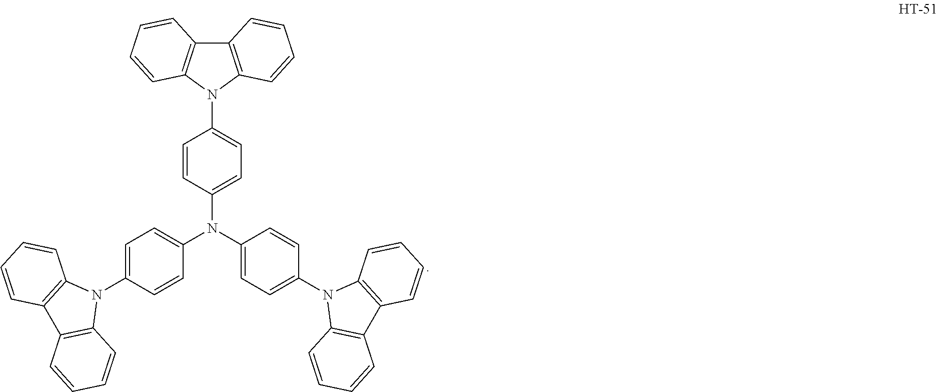 [SMILES: c1ccc2c(c1)c1ccccc1n2-c1ccc(N(c2ccc(-n3c4ccccc4c4ccccc43)cc2)c2ccc(-n3c4ccccc4c4ccccc43)cc2)cc1]

The hole injection layer is located between the anode and the hole transporting layer. The hole injection layer can be a single compound material or a combination of multiple compounds. For example, the hole injection layer can adopt one or more compounds of the above mentioned HT-1 to HT-51, or one or more compounds of the above mentioned HI-1 to HI-3; or one or more compounds of HT-1 to HT-51 doped with one or more compounds of the above mentioned HI-1 to HI-3.

The light emitting layer includes a light emitting dye (i.e. a dopant) that can emit spectra of different wavelengths, and can also include a host material (Host). The light emitting layer may be a monochromatic light emitting layer emitting a single color such as red, green, and blue, etc. The monochromatic light emitting layers with different colors can be arranged in a plane according to the pixel graphics, or stacked together to forma color light emitting layer. When the light emitting layers of different colors are stacked together, they can be separated from each other or connected to each other. The light emitting layer can also be a single color light emitting layer that can emit different colors such as red, green, and blue, etc., at the same time.

According to different technologies, the light emitting layer material can be a phosphorescent electroluminescent material and other various materials.

In one aspect of the disclosure, the phosphorescent electroluminescence technology is adopted for the light emitting layer. The phosphorescent doping material of the light emitting layer thereof can be selected from the group consisting of, but not limited to one or a combination of more of the above mentioned GPD-1 to GPD-47.

In one aspect of the disclosure, the phosphorescent electroluminescence technology is adopted for the light emitting layer. The phosphorescent doping material of the light emitting layer thereof can be selected from the group consisting of, but not limited to one or a combination of more of the above mentioned RPD-1 to RPD-28.

In one aspect of the disclosure, the phosphorescent electroluminescence technology is adopted for the light emitting layer. The phosphorescent doping material of the light emitting layer thereof can be selected from the group consisting of, but not limited to one or a combination of more of the above listed YPD-1 to YPD-11.

The OLED organic material layer may also include an electron transporting region between the light emitting layer and the cathode. The electron transporting region can be an electron transporting layer (ETL) with a single-layer structure, including a single-layer electron transporting layer containing only one compound and a single-layer electron transporting layer containing multiple compounds. The electron transporting region may also be a multilayer structure comprising at least one of an electron injection layer (EIL), an electron transporting layer (ETL), and a hole blocking layer (HBL).

In one aspect of the disclosure, the electron transporting layer material can be selected from the group consisting of, but not limited to one or a combination of more of the above mentioned ET-1 to ET-57 and the ET-58 to ET-73 below.

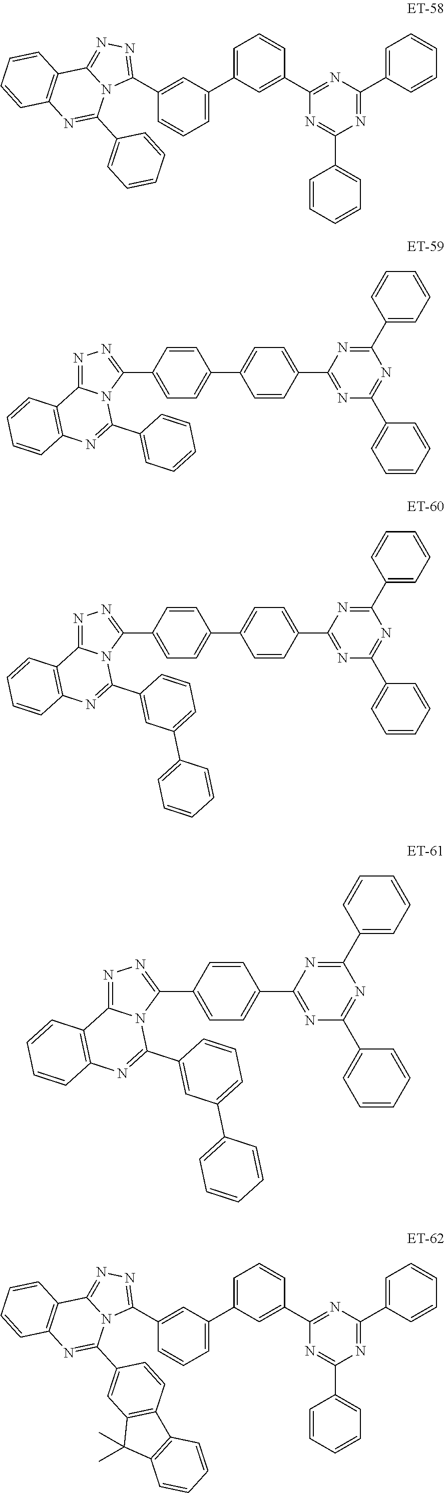 [SMILES: c1ccc(-c2nc(-c3ccccc3)nc(-c3cccc(-c4cccc(-c5nnc6c7ccccc7nc(-c7ccccc7)n56)c4)c3)n2)cc1 ; c1ccc(-c2nc(-c3ccccc3)nc(-c3ccc(-c4ccc(-c5nnc6c7ccccc7nc(-c7ccccc7)n56)cc4)cc3)n2)cc1 ; c1ccc(-c2cccc(-c3nc4ccccc4c4nnc(-c5ccc(-c6ccc(-c7nc(-c8ccccc8)nc(-c8ccccc8)n7)cc6)cc5)n34)c2)cc1 ; c1ccc(-c2cccc(-c3nc4ccccc4c4nnc(-c5ccc(-c6nc(-c7ccccc7)nc(-c7ccccc7)n6)cc5)n34)c2)cc1 ; CC1(C)c2ccccc2-c2ccc(-c3nc4ccccc4c4nnc(-c5cccc(-c6cccc(-c7nc(-c8ccccc8)nc(-c8ccccc8)n7)c6)c5)n34)cc21]

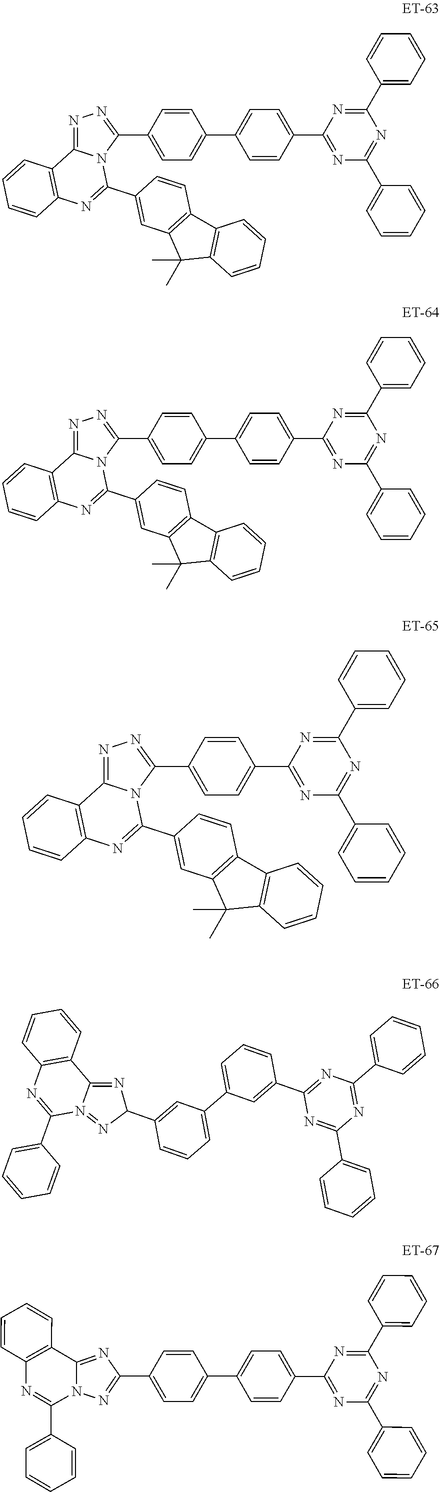

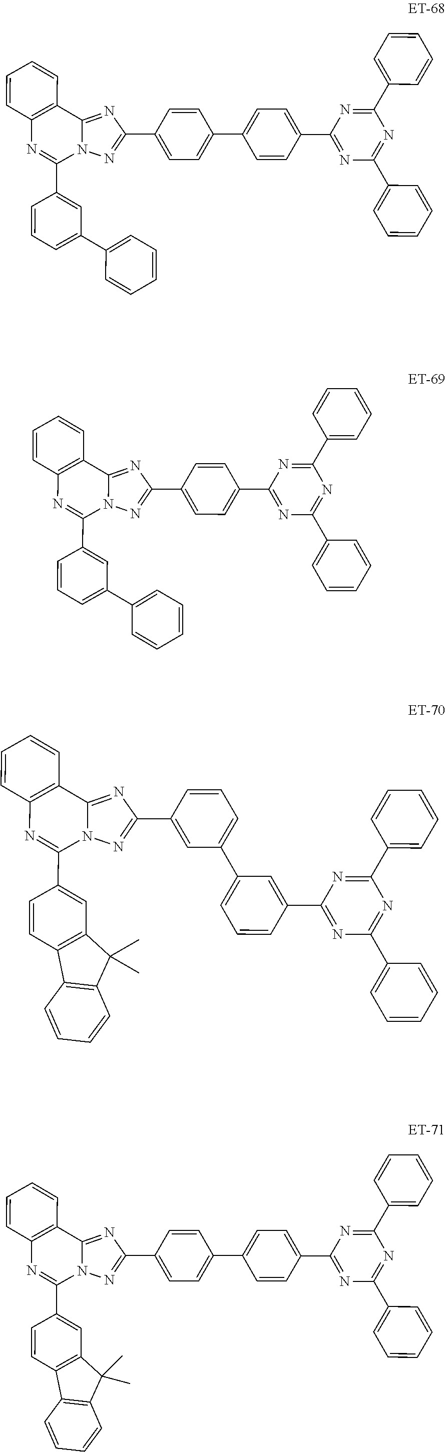 [SMILES: c1ccc(-c2cccc(-c3nc4ccccc4c4nc(-c5ccc(-c6ccc(-c7nc(-c8ccccc8)nc(-c8ccccc8)n7)cc6)cc5)nn34)c2)cc1 ; c1ccc(-c2cccc(-c3nc4ccccc4c4nc(-c5ccc(-c6nc(-c7ccccc7)nc(-c7ccccc7)n6)cc5)nn34)c2)cc1 ; CC1(C)c2ccccc2-c2ccc(-c3nc4ccccc4c4nc(-c5cccc(-c6cccc(-c7nc(-c8ccccc8)nc(-c8ccccc8)n7)c6)c5)nn34)cc21 ; CC1(C)c2ccccc2-c2ccc(-c3nc4ccccc4c4nc(-c5ccc(-c6ccc(-c7nc(-c8ccccc8)nc(-c8ccccc8)n7)cc6)cc5)nn34)cc21]

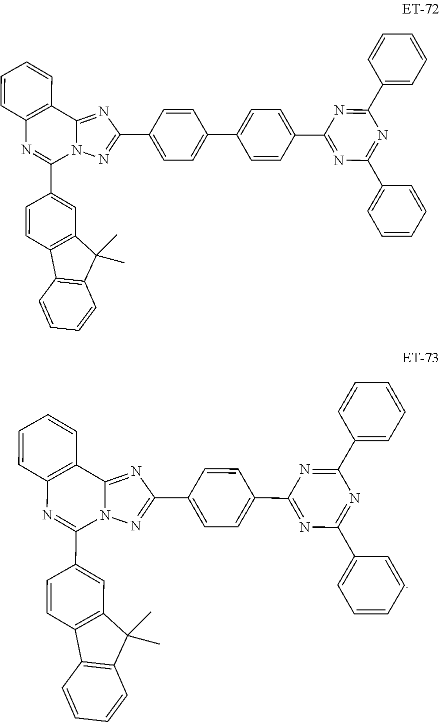 [SMILES: CC1(C)c2ccccc2-c2ccc(-c3nc4ccccc4c4nc(-c5ccc(-c6ccc(-c7nc(-c8ccccc8)nc(-c8ccccc8)n7)cc6)cc5)nn34)cc21 ; CC1(C)c2ccccc2-c2ccc(-c3nc4ccccc4c4nc(-c5ccc(-c6nc(-c7ccccc7)nc(-c7ccccc7)n6)cc5)nn34)cc21]

The device can also include an electron injection layer between the electron transporting layer and the cathode. The electron injection layer material includes, but is not limited to, one or a combination of more of: LiQ, LiF, NaCl, CsF, Li₂O, Cs₂CO₃, BaO, Na, Li, Ca, Mg, Yb as listed below.

In another embodiment of the disclosure, a display device is provided. The display device includes the organic electroluminescent device as described in the first object of the disclosure.

The above preferred embodiments provide an organic electroluminescent device using a dual host light emitting layer, in which the first host material selects the compound as shown in Formula I. Compared with the existing technology, the compound has a high hole mobility and a suitable energy level, and can adjust the carrier distribution inside the light emitting layer, so as to regulate the carrier composite region, and has a high spatial packing structure, when it is used as one of the dual hosts, it is combined with the second host material to precisely control the distribution of carriers inside the light emitting layer, so as to improve the light extraction efficiency of the organic electroluminescent devices, and thus improving the photoelectric performance of the devices. The current efficiency of the organic electroluminescent devices provided by the preferred embodiments above is all above 11.7 cd/A or above, most of which can reach 15 cd/A or above, and the maximum can reach 17 cd/A or above.

According to another aspect of the disclosure, an organic electroluminescent device is provided. The organic electroluminescent device has higher efficiency. The organic electroluminescent device includes an anode layer, a cathode layer and an organic layer arranged between the anode layer and the cathode layer:

the organic layer contains compound I and compound II;

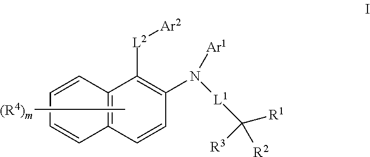

the Ar¹ [symbol] Ar² are independently selected from the group consisting of a substituted or unsubstituted C6-C30 aryl or a substituted or unsubstituted C3-C30 heteroaryl;

the L¹ is selected from the group consisting of a substituted or unsubstituted C6-C30 arylene or a substituted or unsubstituted C3-C30 heteroarylene;

the L² is selected from the group consisting of one of a single bond, a substituted or unsubstituted C6-C30 arylene or a substituted or unsubstituted C3-C30 heteroarylene;

the R¹, R² and R³ are independently selected from the group consisting of any one of a substituted or unsubstituted C1-C20 chain alkyl, a substituted or unsubstituted C3-C20 cycloalkyl, a substituted or unsubstituted C6-C30 arylamino, a substituted or unsubstituted C3-C30 heteroarylamino, a substituted or unsubstituted C6-C30 aryl, a substituted or unsubstituted C3-C30 heteroaryl;

the m is an integer from 0 to 6, for example, 1, 2, 3, 4, 5, etc.;

the R⁴ are independently selected from the group consisting of any one of a substituted or unsubstituted C1-C20 chain alkyl, a substituted or unsubstituted C3-C20 cycloalkyl, a substituted or unsubstituted C6-C30 arylamino, a substituted or unsubstituted C3-C30 heteroarylamino, a substituted or unsubstituted C6-C30 aryl;

the compound II has the structure as shown in Formula (3),

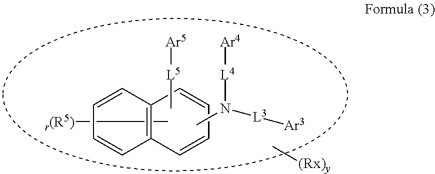

the r is an integer from 0 to 6, for example, 1, 2, 3, 4, 5, etc.;

the Ar³ to Ar⁵ are independently selected from the group consisting of a substituted or unsubstituted C6-C30 aryl or a substituted or unsubstituted C3-C30 heteroaryl;

the L³ to L⁵ are each independently selected from the group consisting of any one of a single bond, a substituted or unsubstituted C6-C30 arylene, and a substituted or unsubstituted C3-C30 heteroarylene;

the R⁵ are independently selected from the group consisting of any one of a substituted or unsubstituted C1-C20 chain alkyl, a substituted or unsubstituted C3-C20 cycloalkyl, a substituted or unsubstituted C6-C30 aryl, and a substituted or unsubstituted C3-C30 heteroaryl;

wherein, Rx is a substituent at any substitutable position, any substitutable position refers to the substitutable position of the structure in the dotted circles, for example, it can be a substitutable position of any one of naphthalene ring, Ar³ to Ar⁵, L³-L⁵ and R⁵; the number of Rx is y, y is an integer from 1 to 15, (e.g., it can be 2, 3, 4, 5, 6, 7, 8, 9, 10, 11, 12, 13, 14, etc.; the Rx is selected from the group consisting of a substituted or unsubstituted C3-C20 cycloalkyl;

In the above Formula I and Formula (3), the substituted group in Ar¹ to Ar⁵, L¹ to L⁵, R¹, R², R³, R⁴, R⁵ and Rx are independently selected from the group consisting of one or a combination of at least two of halogen, C1-C10 chain alkyl, C3-C10 cycloalkyl, C1-C10 alkoxy, C1-C10 thioalkoxy, C6-C30 arylamino, C3-C30 heteroarylamino, C6-C30 monocyclic aryl, C10-C30 fused ring aryl, C3-C30 monocyclic heteroaryl, and C6-C30 fused ring heteroaryl.

The aforementioned “substituted or unsubstituted” groups can be substituted with one substituent or multiple substituents. When the substituents are multiple, they can be selected from different substituents and have the same meaning when the disclosure involves the same expression, and the selection range of the substituents is as shown above and will not be repeated herein.

In the disclosure, the expression of chemical elements includes the concept of isotopes with the same chemical properties. For example, hydrogen (H) includes ¹H (protium or H), ²H (deuterium or D), etc; and carbon (C) includes ¹²C and ¹³C, etc.

In the disclosure, the heteroatom of a heteroaryl group usually refers to one selected from N, O, and S.

In the disclosure, the expression of the ring structure crossed by “—” indicates that the connection site is at any position on the ring structure where bonds can be formed.

The above-mentioned C1-C20 chain alkyl is preferably C1-C10 chain alkyl, more preferably C1-C6 chain alkyl, for example, methyl, ethyl, n-propyl, iso-propyl, n-butyl, iso-butyl, tert-butyl, n-hexyl, n-octyl, n-pentyl, n-heptyl, n-nonyl, n-decyl, etc., can be listed.

The above-mentioned C3-C20 cycloalkyl is preferably cyclopropyl, cyclobutyl, cyclopentyl, and cyclohexyl.

The above-mentioned substituted or unsubstituted C6-C30 aryl(ene), preferably C6-C20 aryl, preferably the aryl is a group consisting of the group of phenyl, biphenyl, terphenyl, naphthyl, anthracyl, phenanthryl, indenyl, fluorenyland derivatives thereof, fluoranthenyl, triphenylene, pyrenyl, perylene, chrysenyl and tetracenyl. The biphenyl is selected from the group consisting of 2-biphenyl, 3-biphenyl and 4-biphenyl; the terphenyl includes para-terphenyl-4-yl, para-terphenyl-3-yl, para-terphenyl-2-yl, meta-terphenyl-4-yl, meta-terphenyl-3-yl and meta-terphenyl-2-yl; the naphthyl includes 1-naphthyl or 2-naphthyl; the anthracyl is selected from the group consisting of 1-anthracyl, 2-anthracyl and 9-anthracyl; the fluorenyl is selected from the group consisting of 1-fluorenyl, 2-fluorenyl, 3-fluorenyl, 4-fluorenyl and 9-fluorenyl; the fluorenyl derivative is selected from the group consisting of 9,9′-dimethylfluorene, 9,9′-spirobifluorene and benzofluorene; the pyrenyl is selected from the group consisting of 1-pyrenyl, 2-pyrenyl and 4-pyrenyl; and the tetracenyl is selected from the group consisting of 1-tetracenyl, 2-tetracenyl and 9-tetracenyl.

In the disclosure, the aryl includes monocyclic aryl and fused ring aryl, the heteroaryl also includes monocyclic heteroaryl and fused ring heteroaryl.

The above mentioned substituted or unsubstituted C3-C30 heteroaryl(ene), preferably C4-C20 heteroaryl, preferably the heteroaryl is furanyl, thienyl, pyrrolyl, benzofuranyl, benzothienyl, isobenzofuranyl, indolyl, dibenzofuranyl, dibenzothiophenyl, carbazolyl and derivatives thereof, wherein the carbazolyl derivative is preferably 9-phenylcarbazole, 9-naphthylcarbazolebenzocarbazole, dibenzocarbazole, or indolocarbazole.

The above mentioned C6-C30 arylamino refers to a group formed by connecting aryl to amino, the connecting bond can be on the amino group or on the aryl group. The C3-C30 heteroaryl amino group is in a similar way.

In this embodiment, compound I and compound II are used as the materials of the organic layer at the same time, wherein compound I can adjust the distribution of carriers inside the light emitting layer, so as to regulate the carrier composite region, and it also has a higher spatial packing structure. At the same time, compound II has a higher molecular plane unfolding properties, so as to achieve faster transferring of holes, improve hole mobility, and has a higher triplet energy level, which can block excess excitons. The combination of compound I and compound II can effectively improve the efficiency of the devices.

Preferably, the compound I has any one of the structures as shown in the above mentioned P1-P777.

Preferably, the compound II has any one of the structures as shown in A1 to A291:

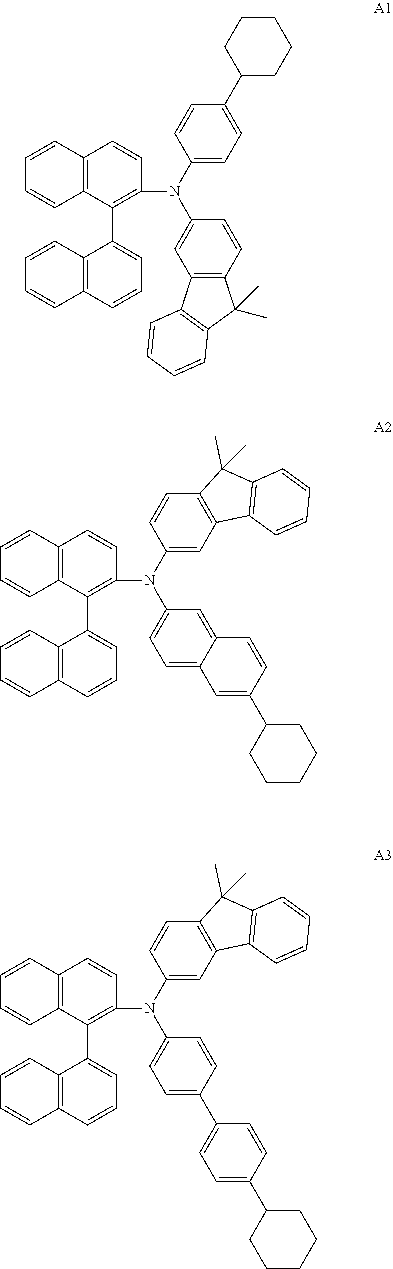 [SMILES: CC1(C)c2ccccc2-c2cc(N(c3ccc(C4CCCCC4)cc3)c3ccc4ccccc4c3-c3cccc4ccccc34)ccc21 ; CC1(C)c2ccccc2-c2cc(N(c3ccc4cc(C5CCCCC5)ccc4c3)c3ccc4ccccc4c3-c3cccc4ccccc34)ccc21 ; CC1(C)c2ccccc2-c2cc(N(c3ccc(-c4ccc(C5CCCCC5)cc4)cc3)c3ccc4ccccc4c3-c3cccc4ccccc34)ccc21]

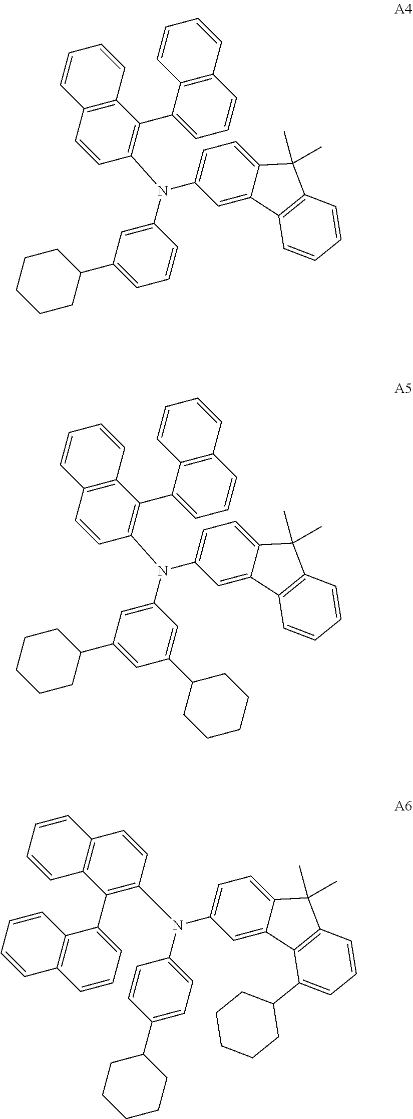 [SMILES: CC1(C)c2ccccc2-c2cc(N(c3cccc(C4CCCCC4)c3)c3ccc4ccccc4c3-c3cccc4ccccc34)ccc21 ; CC1(C)c2ccccc2-c2cc(N(c3cc(C4CCCCC4)cc(C4CCCCC4)c3)c3ccc4ccccc4c3-c3cccc4ccccc34)ccc21 ; CC1(C)c2ccc(N(c3ccc(C4CCCCC4)cc3)c3ccc4ccccc4c3-c3cccc4ccccc34)cc2-c2c(C3CCCCC3)cccc21]

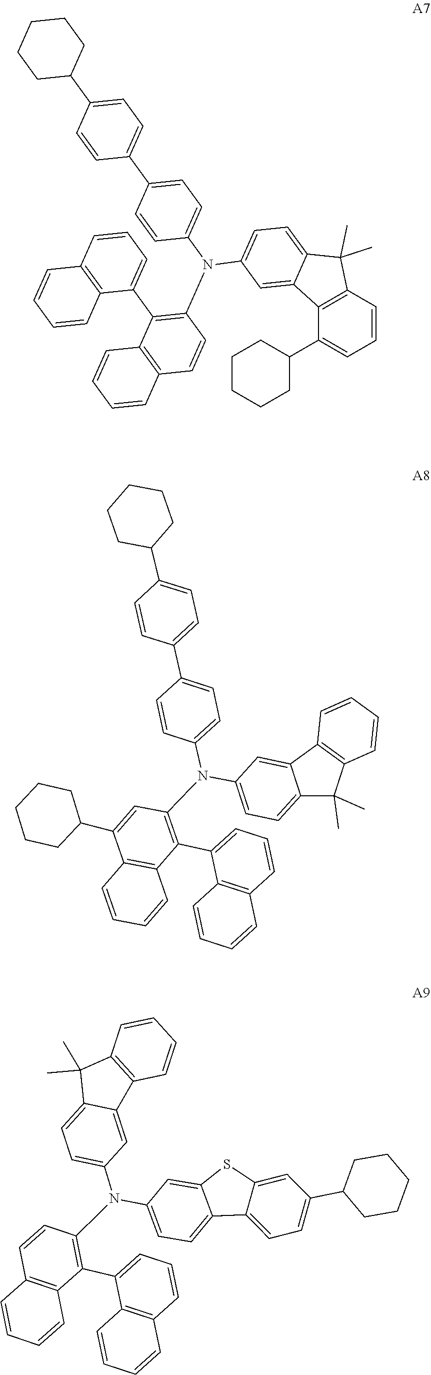 [SMILES: CC1(C)c2ccc(N(c3ccc(-c4ccc(C5CCCCC5)cc4)cc3)c3ccc4ccccc4c3-c3cccc4ccccc34)cc2-c2c(C3CCCCC3)cccc21 ; CC1(C)c2ccccc2-c2cc(N(c3ccc(-c4ccc(C5CCCCC5)cc4)cc3)c3cc(C4CCCCC4)c4ccccc4c3-c3cccc4ccccc34)ccc21 ; CC1(C)c2ccccc2-c2cc(N(c3ccc4c(c3)sc3cc(C5CCCCC5)ccc34)c3ccc4ccccc4c3-c3cccc4ccccc34)ccc21]

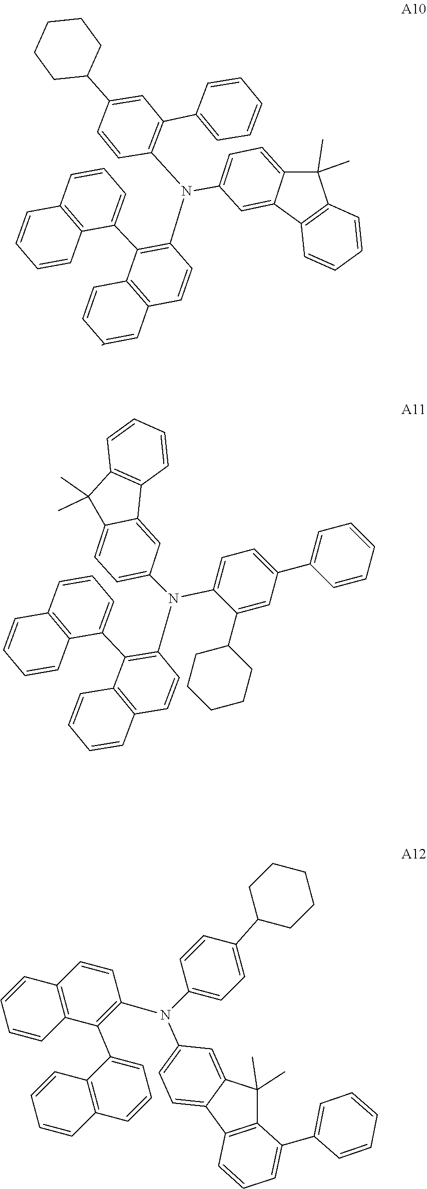 [SMILES: Cc1ccc2c(-c3cccc4ccccc34)c(N(c3ccc4c(c3)-c3ccccc3C4(C)C)c3ccc(C4CCCCC4)cc3-c3ccccc3)ccc2c1 ; CC1(C)c2ccccc2-c2cc(N(c3ccc(-c4ccccc4)cc3C3CCCCC3)c3ccc4ccccc4c3-c3cccc4ccccc34)ccc21 ; CC1(C)c2cc(N(c3ccc(C4CCCCC4)cc3)c3ccc4ccccc4c3-c3cccc4ccccc34)ccc2-c2cccc(-c3ccccc3)c21]

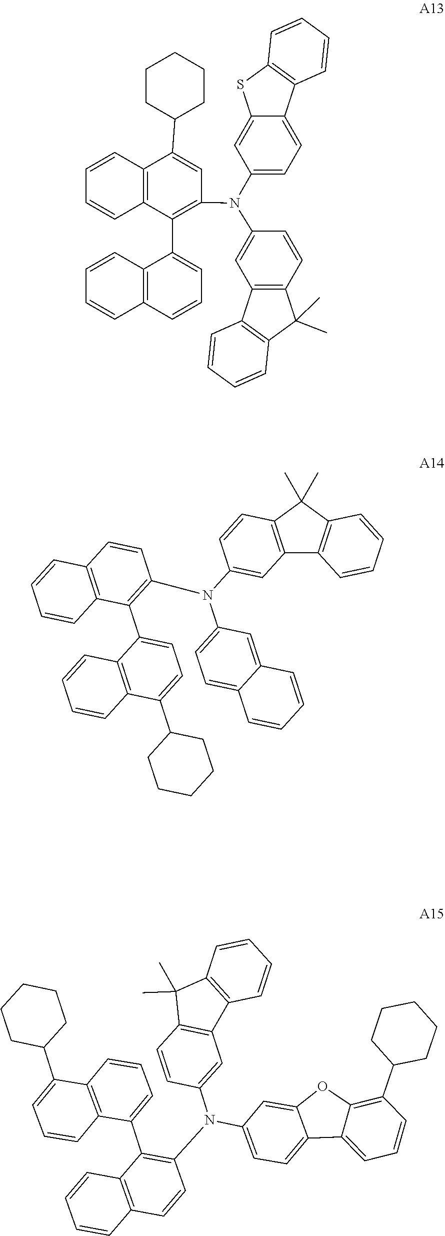 [SMILES: CC1(C)c2ccccc2-c2cc(N(c3ccc4c(c3)sc3ccccc34)c3cc(C4CCCCC4)c4ccccc4c3-c3cccc4ccccc34)ccc21 ; CC1(C)c2ccccc2-c2cc(N(c3ccc4ccccc4c3)c3ccc4ccccc4c3-c3ccc(C4CCCCC4)c4ccccc34)ccc21 ; CC1(C)c2ccccc2-c2cc(N(c3ccc4c(c3)oc3c(C5CCCCC5)cccc34)c3ccc4ccccc4c3-c3cccc4c(C5CCCCC5)cccc34)ccc21]

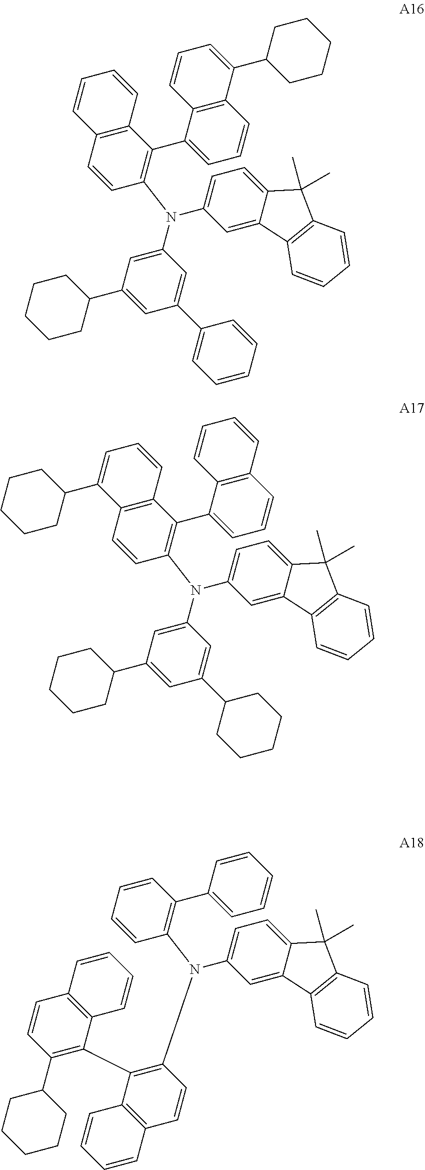

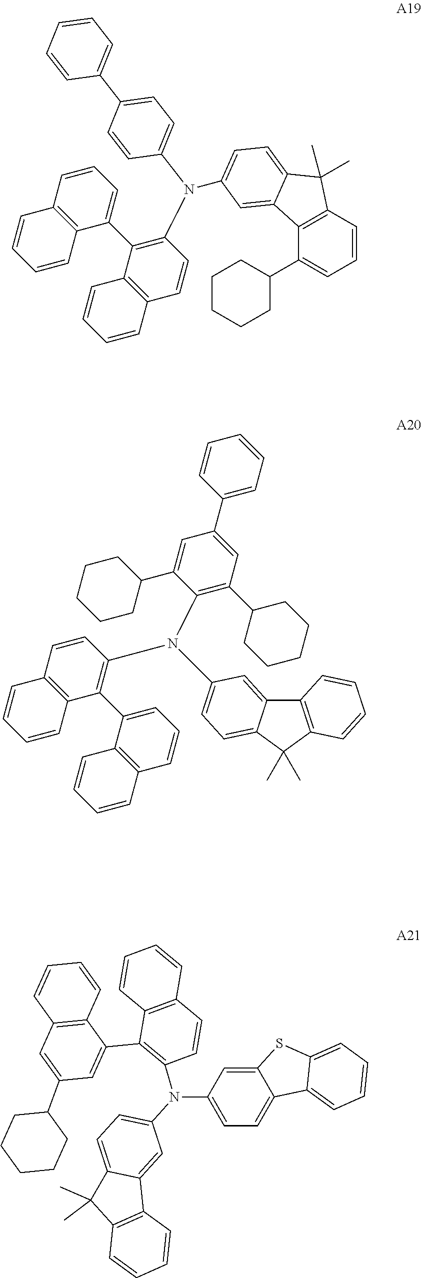 [SMILES: CC1(C)c2ccc(N(c3ccc(-c4ccccc4)cc3)c3ccc4ccccc4c3-c3cccc4ccccc34)cc2-c2c(C3CCCCC3)cccc21 ; CC1(C)c2ccccc2-c2cc(N(c3ccc4ccccc4c3-c3cccc4ccccc34)c3c(C4CCCCC4)cc(-c4ccccc4)cc3C3CCCCC3)ccc21 ; CC1(C)c2ccccc2-c2cc(N(c3ccc4c(c3)sc3ccccc34)c3ccc4ccccc4c3-c3cc(C4CCCCC4)cc4ccccc34)ccc21]

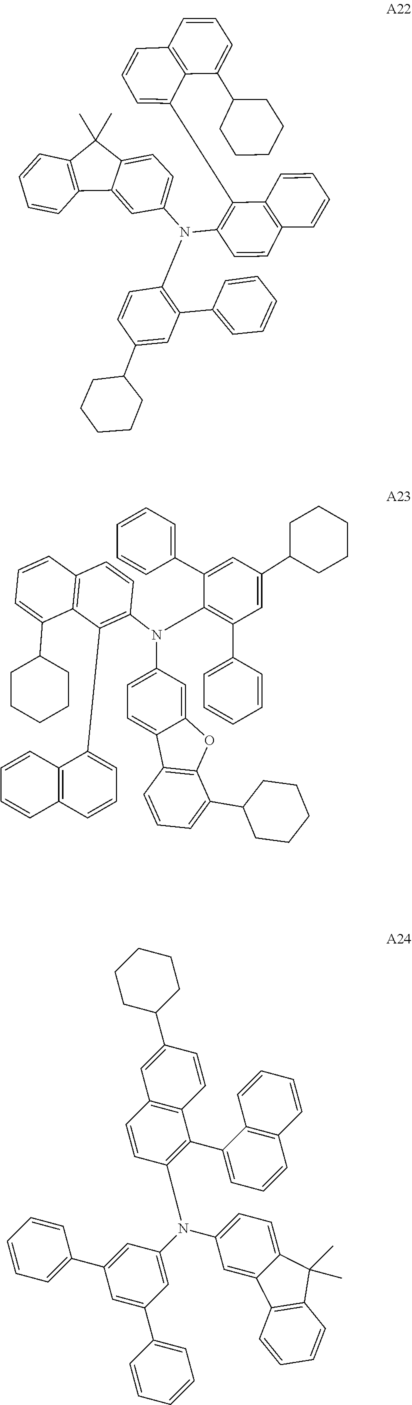 [SMILES: CC1(C)c2ccccc2-c2cc(N(c3ccc(C4CCCCC4)cc3-c3ccccc3)c3ccc4ccccc4c3-c3cccc4cccc(C5CCCCC5)c34)ccc21 ; c1ccc(-c2cc(C3CCCCC3)cc(-c3ccccc3)c2N(c2ccc3c(c2)oc2c(C4CCCCC4)cccc23)c2ccc3cccc(C4CCCCC4)c3c2-c2cccc3ccccc23)cc1 ; CC1(C)c2ccccc2-c2cc(N(c3cc(-c4ccccc4)cc(-c4ccccc4)c3)c3ccc4cc(C5CCCCC5)ccc4c3-c3cccc4ccccc34)ccc21]

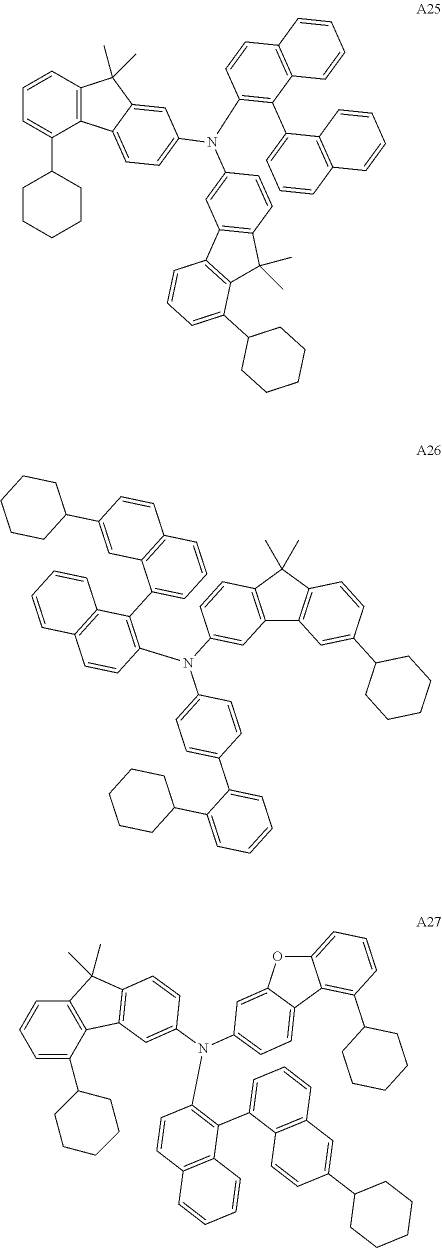 [SMILES: CC1(C)c2cc(N(c3ccc4c(c3)-c3cccc(C5CCCCC5)c3C4(C)C)c3ccc4ccccc4c3-c3cccc4ccccc34)ccc2-c2c(C3CCCCC3)cccc21 ; CC1(C)c2ccc(C3CCCCC3)cc2-c2cc(N(c3ccc(-c4ccccc4C4CCCCC4)cc3)c3ccc4ccccc4c3-c3cccc4ccc(C5CCCCC5)cc34)ccc21 ; CC1(C)c2ccc(N(c3ccc4c(c3)oc3cccc(C5CCCCC5)c34)c3ccc4ccccc4c3-c3cccc4cc(C5CCCCC5)ccc34)cc2-c2c(C3CCCCC3)cccc21]

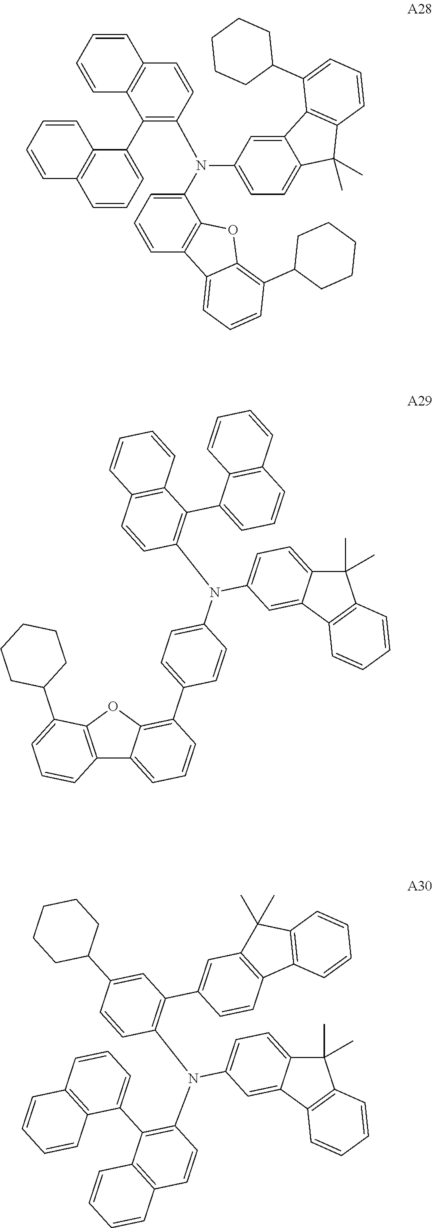 [SMILES: CC1(C)c2ccc(N(c3ccc4ccccc4c3-c3cccc4ccccc34)c3cccc4c3oc3c(C5CCCCC5)cccc34)cc2-c2c(C3CCCCC3)cccc21 ; CC1(C)c2ccccc2-c2cc(N(c3ccc(-c4cccc5c4oc4c(C6CCCCC6)cccc45)cc3)c3ccc4ccccc4c3-c3cccc4ccccc34)ccc21 ; CC1(C)c2ccccc2-c2cc(N(c3ccc(C4CCCCC4)cc3-c3ccc4c(c3)C(C)(C)c3ccccc3-4)c3ccc4ccccc4c3-c3cccc4ccccc34)ccc21]

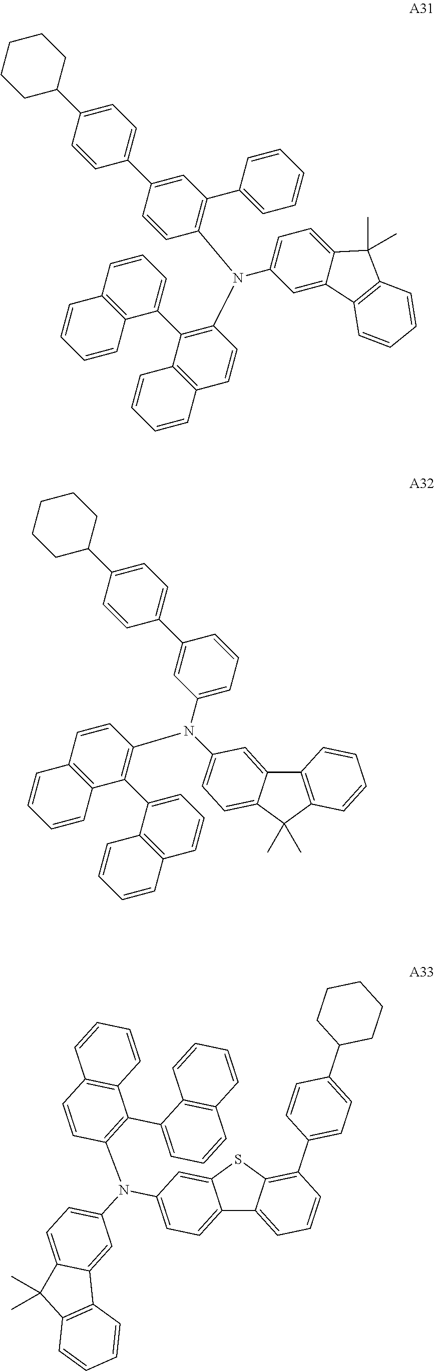 [SMILES: CC1(C)c2ccccc2-c2cc(N(c3ccc(-c4ccc(C5CCCCC5)cc4)cc3-c3ccccc3)c3ccc4ccccc4c3-c3cccc4ccccc34)ccc21 ; CC1(C)c2ccccc2-c2cc(N(c3cccc(-c4ccc(C5CCCCC5)cc4)c3)c3ccc4ccccc4c3-c3cccc4ccccc34)ccc21 ; CC1(C)c2ccccc2-c2cc(N(c3ccc4c(c3)sc3c(-c5ccc(C6CCCCC6)cc5)cccc34)c3ccc4ccccc4c3-c3cccc4ccccc34)ccc21]

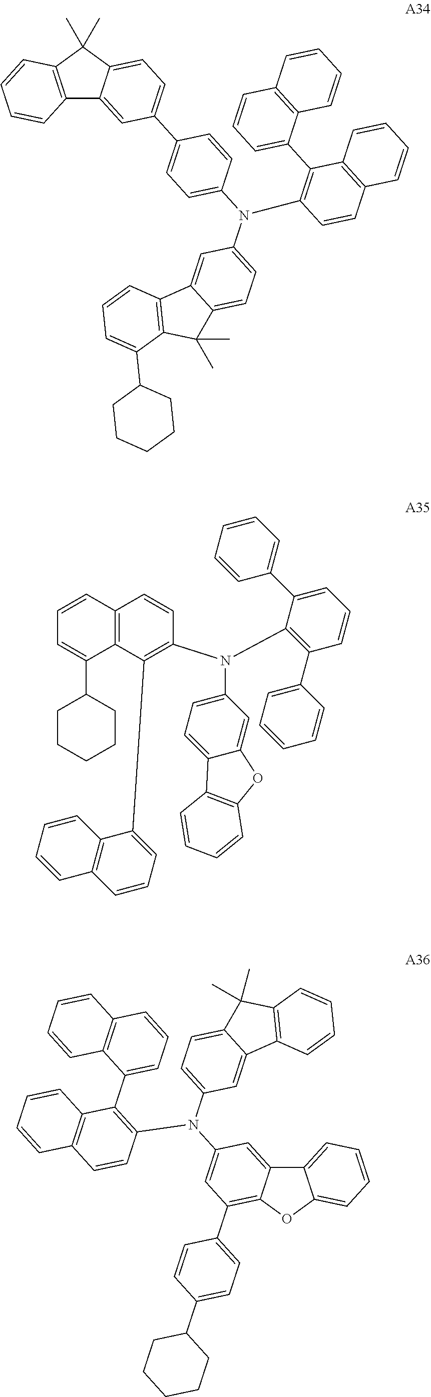 [SMILES: CC1(C)c2ccccc2-c2cc(-c3ccc(N(c4ccc5c(c4)-c4cccc(C6CCCCC6)c4C5(C)C)c4ccc5ccccc5c4-c4cccc5ccccc45)cc3)ccc21 ; c1ccc(-c2cccc(-c3ccccc3)c2N(c2ccc3c(c2)oc2ccccc23)c2ccc3cccc(C4CCCCC4)c3c2-c2cccc3ccccc23)cc1 ; CC1(C)c2ccccc2-c2cc(N(c3cc(-c4ccc(C5CCCCC5)cc4)c4oc5ccccc5c4c3)c3ccc4ccccc4c3-c3cccc4ccccc34)ccc21]

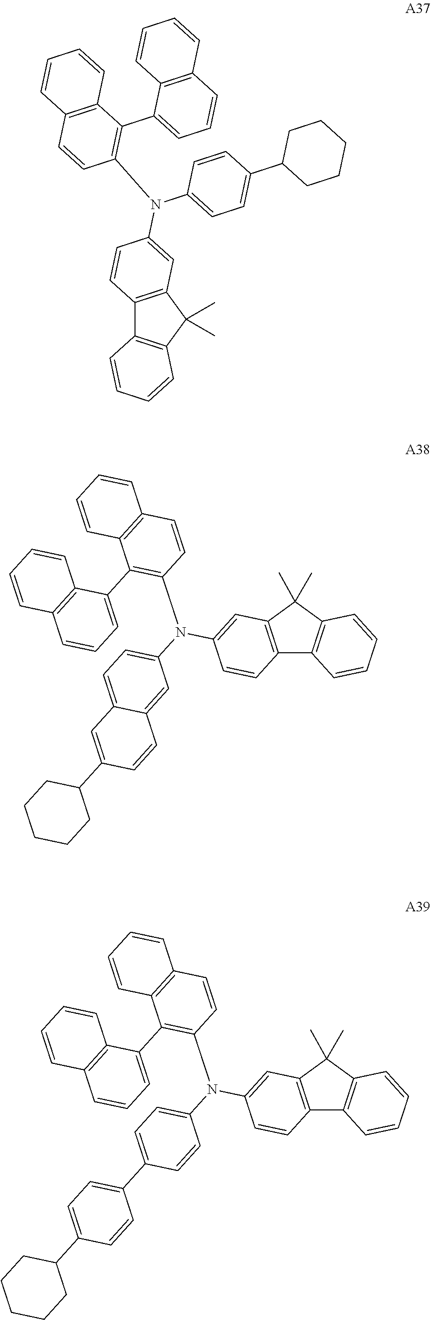 [SMILES: CC1(C)c2ccccc2-c2ccc(N(c3ccc(C4CCCCC4)cc3)c3ccc4ccccc4c3-c3cccc4ccccc34)cc21 ; CC1(C)c2ccccc2-c2ccc(N(c3ccc4cc(C5CCCCC5)ccc4c3)c3ccc4ccccc4c3-c3cccc4ccccc34)cc21 ; CC1(C)c2ccccc2-c2ccc(N(c3ccc(-c4ccc(C5CCCCC5)cc4)cc3)c3ccc4ccccc4c3-c3cccc4ccccc34)cc21]

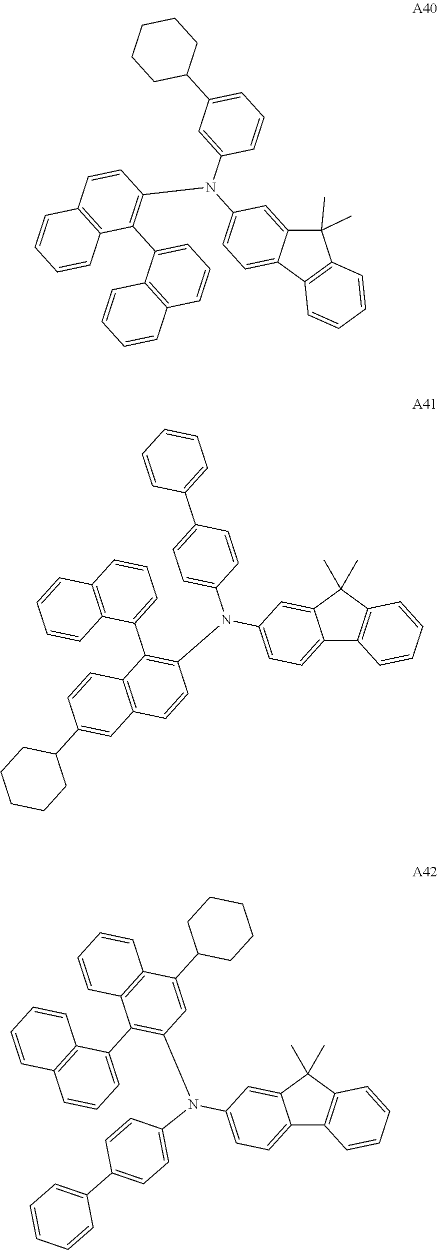 [SMILES: CC1(C)c2ccccc2-c2ccc(N(c3cccc(C4CCCCC4)c3)c3ccc4ccccc4c3-c3cccc4ccccc34)cc21 ; CC1(C)c2ccccc2-c2ccc(N(c3ccc(-c4ccccc4)cc3)c3ccc4cc(C5CCCCC5)ccc4c3-c3cccc4ccccc34)cc21 ; CC1(C)c2ccccc2-c2ccc(N(c3ccc(-c4ccccc4)cc3)c3cc(C4CCCCC4)c4ccccc4c3-c3cccc4ccccc34)cc21]

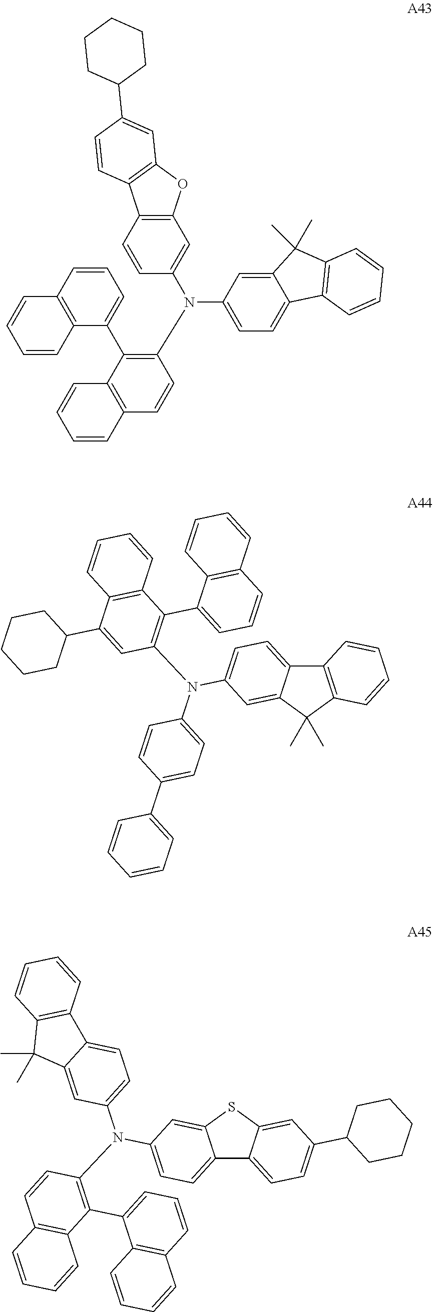 [SMILES: CC1(C)c2ccccc2-c2ccc(N(c3ccc4c(c3)oc3cc(C5CCCCC5)ccc34)c3ccc4ccccc4c3-c3cccc4ccccc34)cc21 ; CC1(C)c2ccccc2-c2ccc(N(c3ccc(-c4ccccc4)cc3)c3cc(C4CCCCC4)c4ccccc4c3-c3cccc4ccccc34)cc21 ; CC1(C)c2ccccc2-c2ccc(N(c3ccc4c(c3)sc3cc(C5CCCCC5)ccc34)c3ccc4ccccc4c3-c3cccc4ccccc34)cc21]

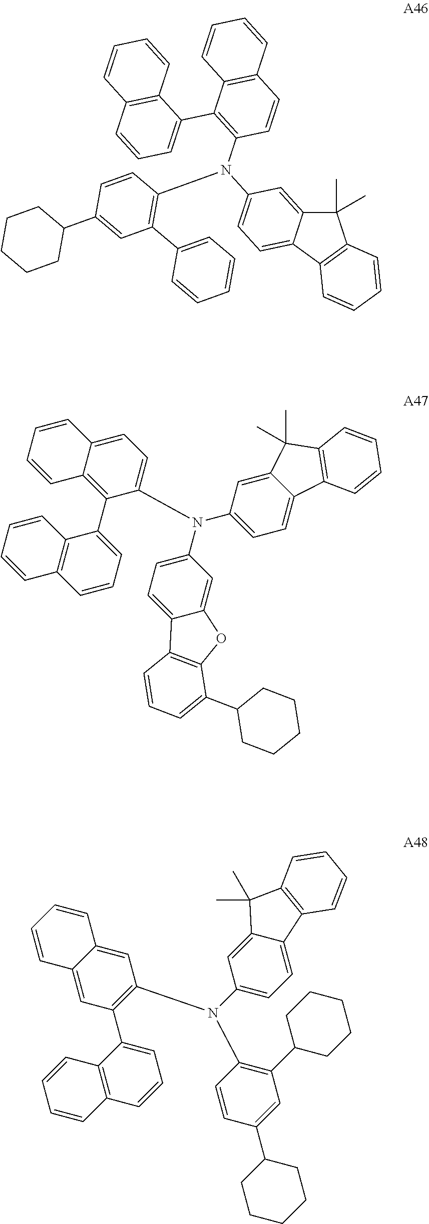 [SMILES: CC1(C)c2ccccc2-c2ccc(N(c3ccc(C4CCCCC4)cc3-c3ccccc3)c3ccc4ccccc4c3-c3cccc4ccccc34)cc21 ; CC1(C)c2ccccc2-c2ccc(N(c3ccc4c(c3)oc3c(C5CCCCC5)cccc34)c3ccc4ccccc4c3-c3cccc4ccccc34)cc21 ; CC1(C)c2ccccc2-c2ccc(N(c3cc4ccccc4cc3-c3cccc4ccccc34)c3ccc(C4CCCCC4)cc3C3CCCCC3)cc21]

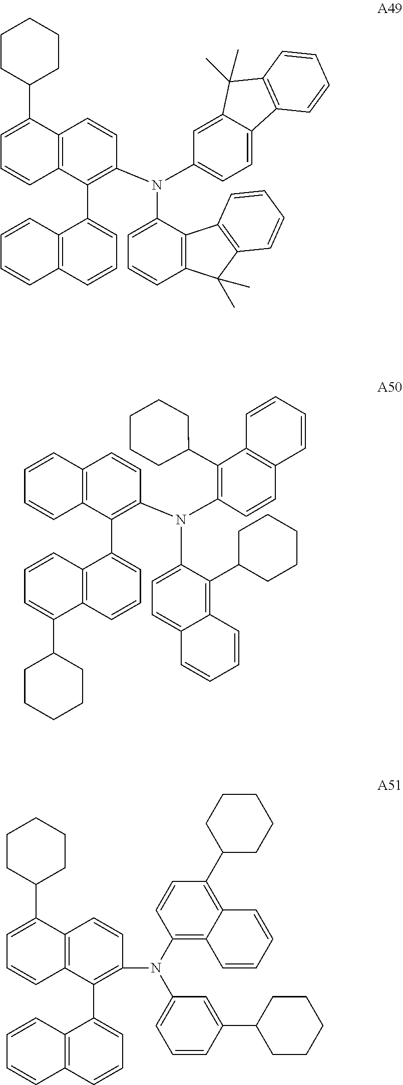 [SMILES: CC1(C)c2ccccc2-c2ccc(N(c3cccc4c3-c3ccccc3C4(C)C)c3ccc4c(C5CCCCC5)cccc4c3-c3cccc4ccccc34)cc21 ; c1ccc2c(-c3cccc4c(C5CCCCC5)cccc34)c(N(c3ccc4ccccc4c3C3CCCCC3)c3ccc4ccccc4c3C3CCCCC3)ccc2c1 ; c1cc(C2CCCCC2)cc(N(c2ccc3c(C4CCCCC4)cccc3c2-c2cccc3ccccc23)c2ccc(C3CCCCC3)c3ccccc23)c1]

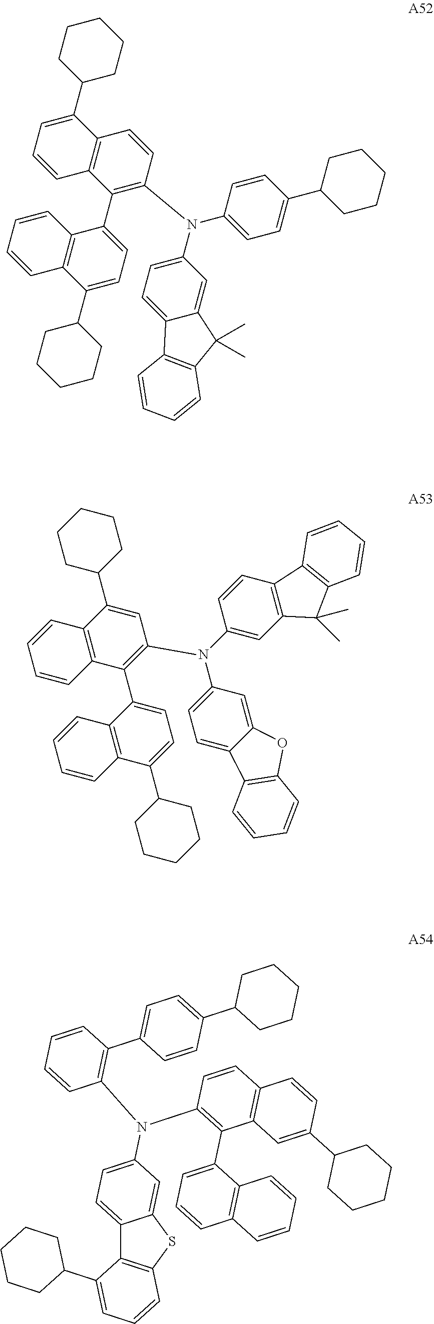 [SMILES: CC1(C)c2ccccc2-c2ccc(N(c3ccc(C4CCCCC4)cc3)c3ccc4c(C5CCCCC5)cccc4c3-c3ccc(C4CCCCC4)c4ccccc34)cc21 ; CC1(C)c2ccccc2-c2ccc(N(c3ccc4c(c3)oc3ccccc34)c3cc(C4CCCCC4)c4ccccc4c3-c3ccc(C4CCCCC4)c4ccccc34)cc21 ; c1ccc(N(c2ccc3c(c2)sc2cccc(C4CCCCC4)c23)c2ccc3ccc(C4CCCCC4)cc3c2-c2cccc3ccccc23)c(-c2ccc(C3CCCCC3)cc2)c1]

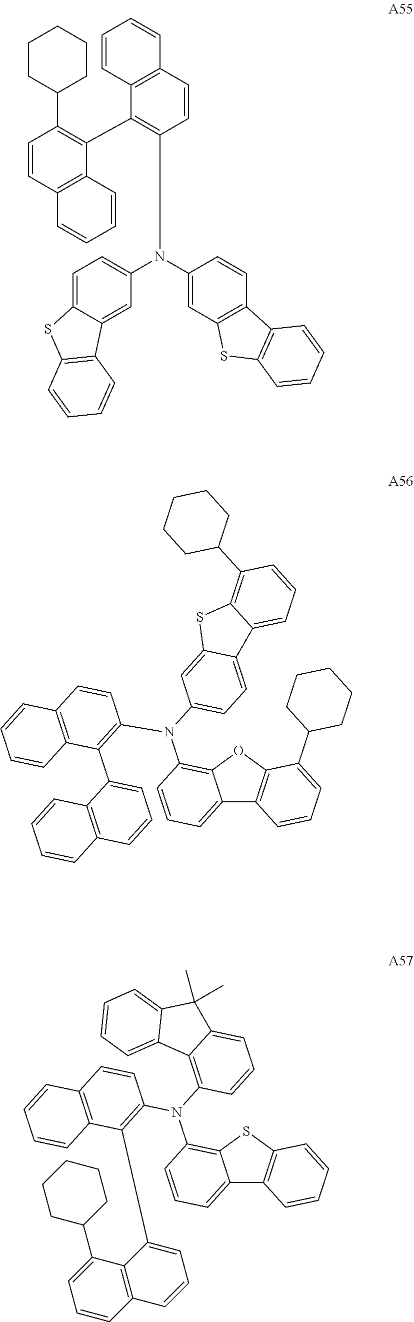

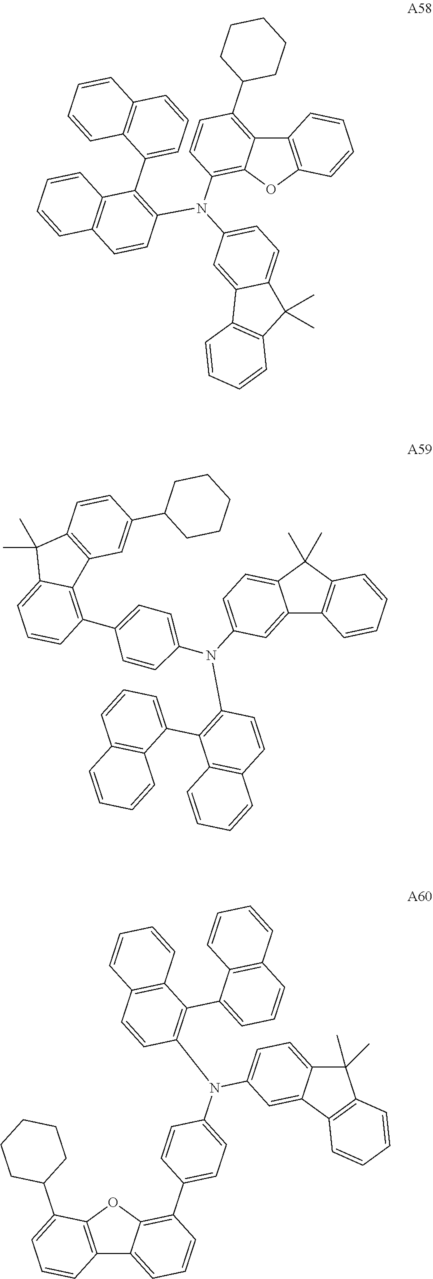 [SMILES: CC1(C)c2ccccc2-c2cc(N(c3ccc4ccccc4c3-c3cccc4ccccc34)c3ccc(C4CCCCC4)c4c3oc3ccccc34)ccc21 ; CC1(C)c2ccccc2-c2cc(N(c3ccc(-c4cccc5c4-c4cc(C6CCCCC6)ccc4C5(C)C)cc3)c3ccc4ccccc4c3-c3cccc4ccccc34)ccc21 ; CC1(C)c2ccccc2-c2cc(N(c3ccc(-c4cccc5c4oc4c(C6CCCCC6)cccc45)cc3)c3ccc4ccccc4c3-c3cccc4ccccc34)ccc21]

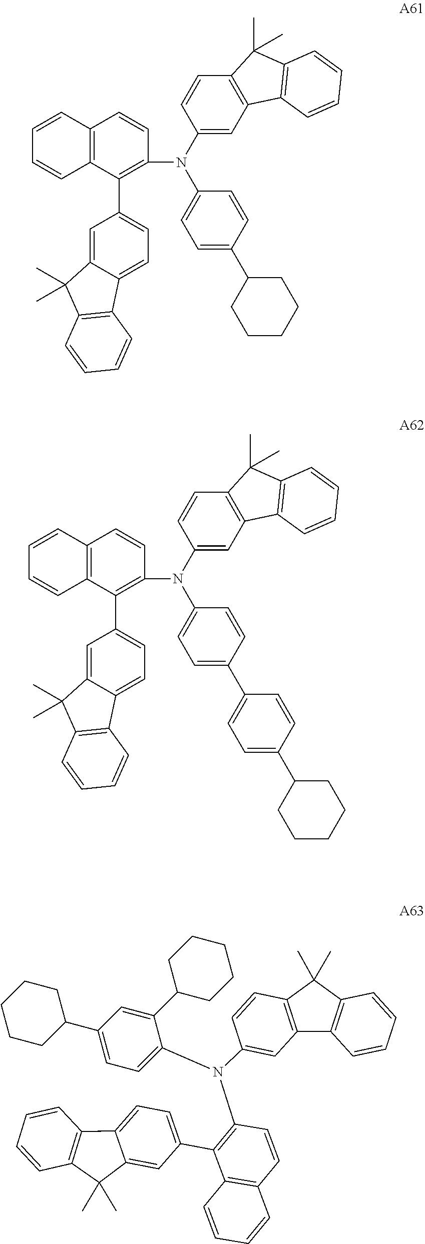 [SMILES: CC1(C)c2ccccc2-c2cc(N(c3ccc(C4CCCCC4)cc3)c3ccc4ccccc4c3-c3ccc4c(c3)C(C)(C)c3ccccc3-4)ccc21 ; CC1(C)c2ccccc2-c2cc(N(c3ccc(-c4ccc(C5CCCCC5)cc4)cc3)c3ccc4ccccc4c3-c3ccc4c(c3)C(C)(C)c3ccccc3-4)ccc21 ; CC1(C)c2ccccc2-c2cc(N(c3ccc(C4CCCCC4)cc3C3CCCCC3)c3ccc4ccccc4c3-c3ccc4c(c3)C(C)(C)c3ccccc3-4)ccc21]

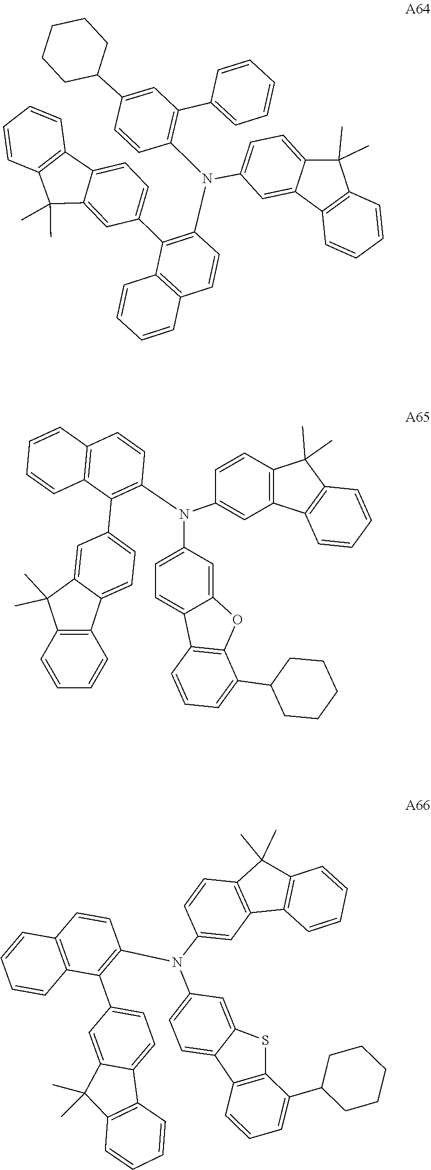 [SMILES: CC1(C)c2ccccc2-c2cc(N(c3ccc(C4CCCCC4)cc3-c3ccccc3)c3ccc4ccccc4c3-c3ccc4c(c3)C(C)(C)c3ccccc3-4)ccc21 ; CC1(C)c2ccccc2-c2cc(N(c3ccc4c(c3)oc3c(C5CCCCC5)cccc34)c3ccc4ccccc4c3-c3ccc4c(c3)C(C)(C)c3ccccc3-4)ccc21 ; CC1(C)c2ccccc2-c2cc(N(c3ccc4c(c3)sc3c(C5CCCCC5)cccc34)c3ccc4ccccc4c3-c3ccc4c(c3)C(C)(C)c3ccccc3-4)ccc21]

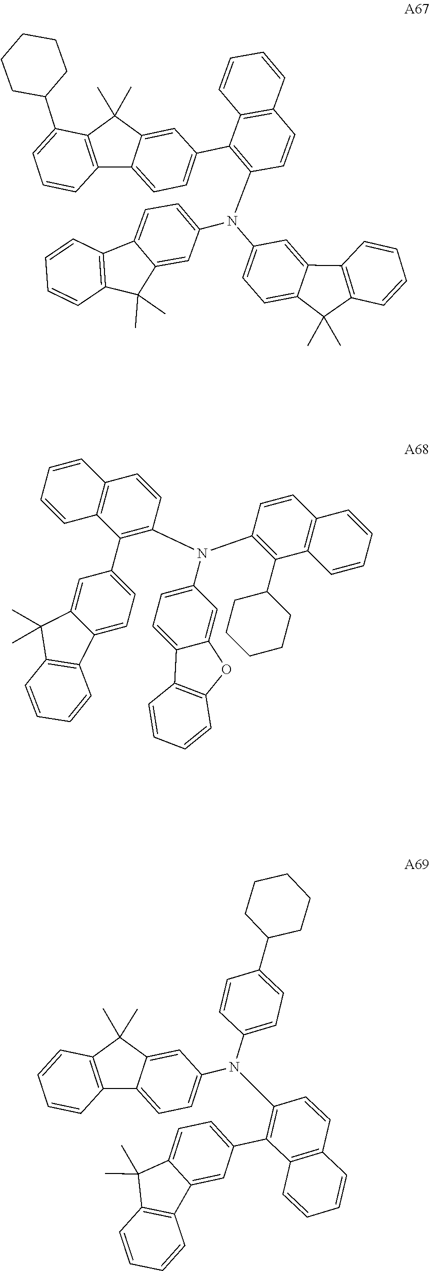 [SMILES: CC1(C)c2ccccc2-c2cc(N(c3ccc4c(c3)C(C)(C)c3ccccc3-4)c3ccc4ccccc4c3-c3ccc4c(c3)C(C)(C)c3c-4cccc3C3CCCCC3)ccc21 ; CC1(C)c2ccccc2-c2ccc(-c3c(N(c4ccc5c(c4)oc4ccccc45)c4ccc5ccccc5c4C4CCCCC4)ccc4ccccc34)cc21 ; CC1(C)c2ccccc2-c2cc(-c3c(N(c4ccc(C5CCCCC5)cc4)c4ccc5c(c4)C(C)(C)c4ccccc4-5)ccc4ccccc34)ccc21]

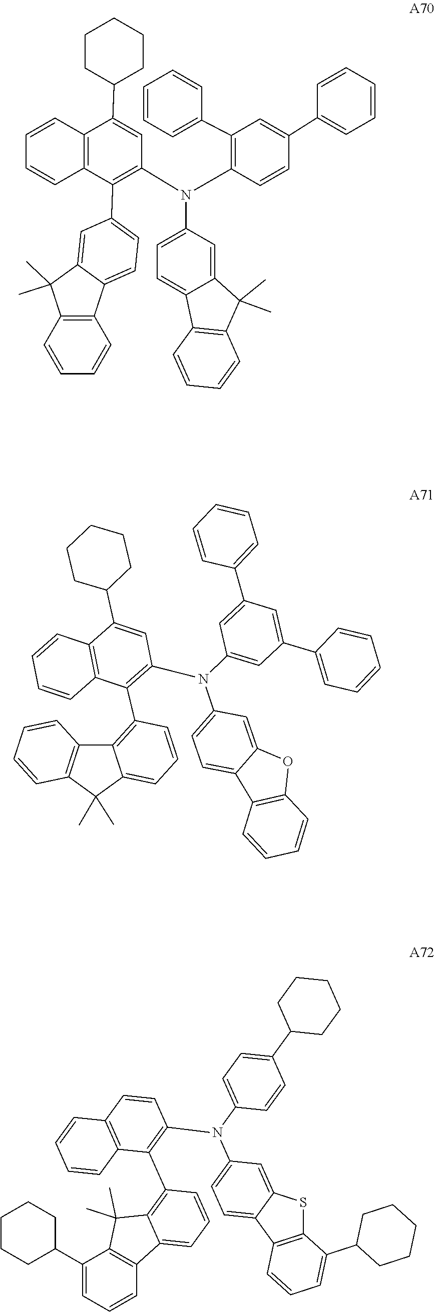 [SMILES: CC1(C)c2ccccc2-c2ccc(-c3c(N(c4ccc5c(c4)C(C)(C)c4ccccc4-5)c4ccc(-c5ccccc5)cc4-c4ccccc4)cc(C4CCCCC4)c4ccccc34)cc21 ; CC1(C)c2ccccc2-c2c(-c3c(N(c4cc(-c5ccccc5)cc(-c5ccccc5)c4)c4ccc5c(c4)oc4ccccc45)cc(C4CCCCC4)c4ccccc34)cccc21 ; CC1(C)c2c(cccc2-c2c(N(c3ccc(C4CCCCC4)cc3)c3ccc4c(c3)sc3c(C5CCCCC5)cccc34)ccc3ccccc23)-c2cccc(C3CCCCC3)c21]

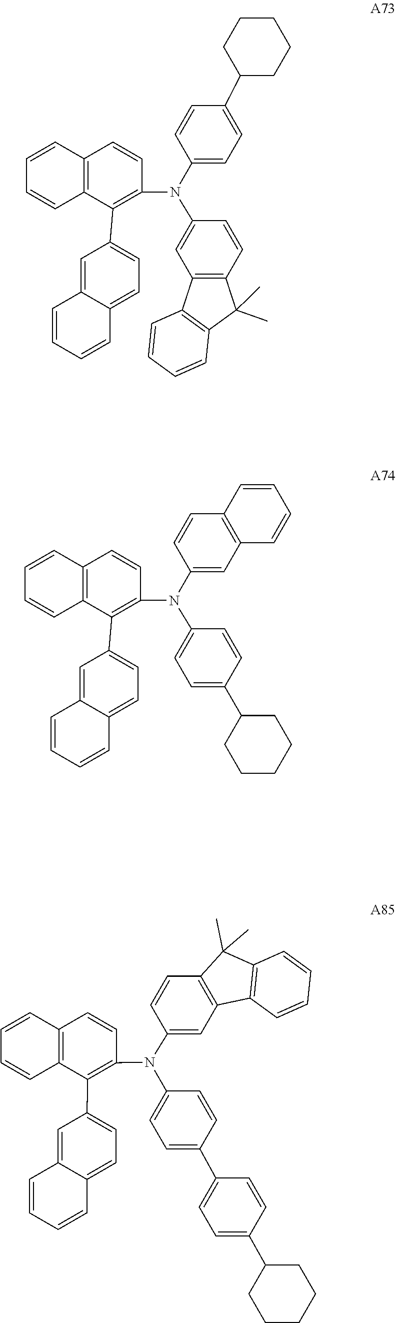 [SMILES: CC1(C)c2ccccc2-c2cc(N(c3ccc(C4CCCCC4)cc3)c3ccc4ccccc4c3-c3ccc4ccccc4c3)ccc21 ; c1ccc2cc(-c3c(N(c4ccc(C5CCCCC5)cc4)c4ccc5ccccc5c4)ccc4ccccc34)ccc2c1 ; CC1(C)c2ccccc2-c2cc(N(c3ccc(-c4ccc(C5CCCCC5)cc4)cc3)c3ccc4ccccc4c3-c3ccc4ccccc4c3)ccc21]

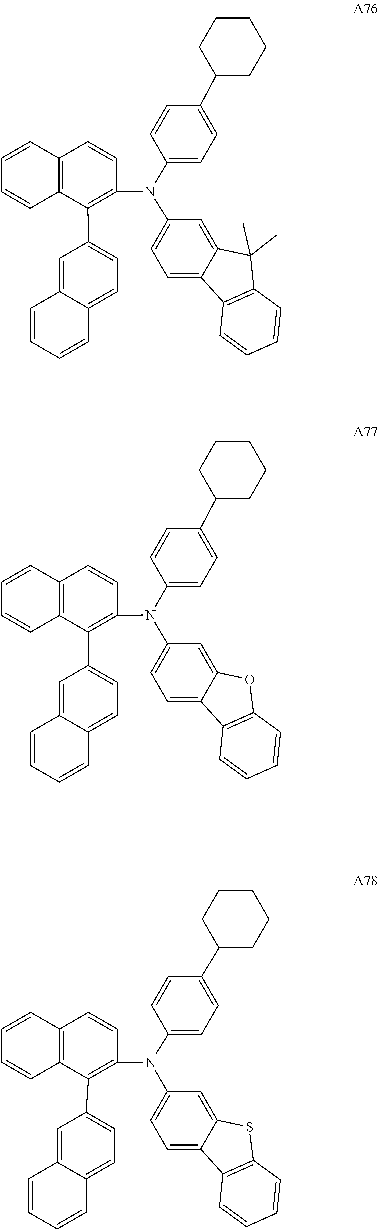 [SMILES: CC1(C)c2ccccc2-c2ccc(N(c3ccc(C4CCCCC4)cc3)c3ccc4ccccc4c3-c3ccc4ccccc4c3)cc21 ; c1ccc2cc(-c3c(N(c4ccc(C5CCCCC5)cc4)c4ccc5c(c4)oc4ccccc45)ccc4ccccc34)ccc2c1 ; c1ccc2cc(-c3c(N(c4ccc(C5CCCCC5)cc4)c4ccc5c(c4)sc4ccccc45)ccc4ccccc34)ccc2c1]

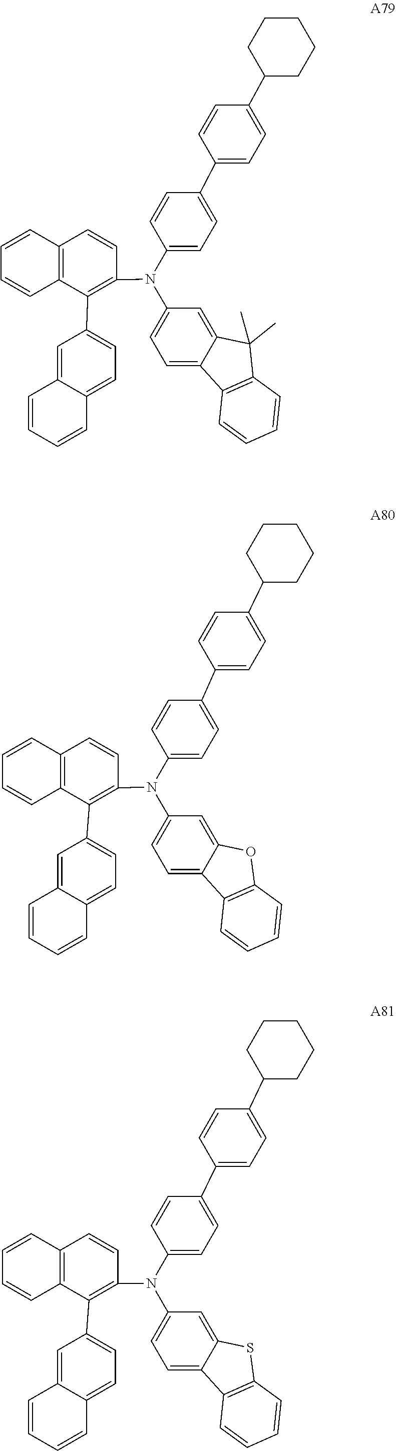 [SMILES: CC1(C)c2ccccc2-c2ccc(N(c3ccc(-c4ccc(C5CCCCC5)cc4)cc3)c3ccc4ccccc4c3-c3ccc4ccccc4c3)cc21 ; c1ccc2cc(-c3c(N(c4ccc(-c5ccc(C6CCCCC6)cc5)cc4)c4ccc5c(c4)oc4ccccc45)ccc4ccccc34)ccc2c1 ; c1ccc2cc(-c3c(N(c4ccc(-c5ccc(C6CCCCC6)cc5)cc4)c4ccc5c(c4)sc4ccccc45)ccc4ccccc34)ccc2c1]

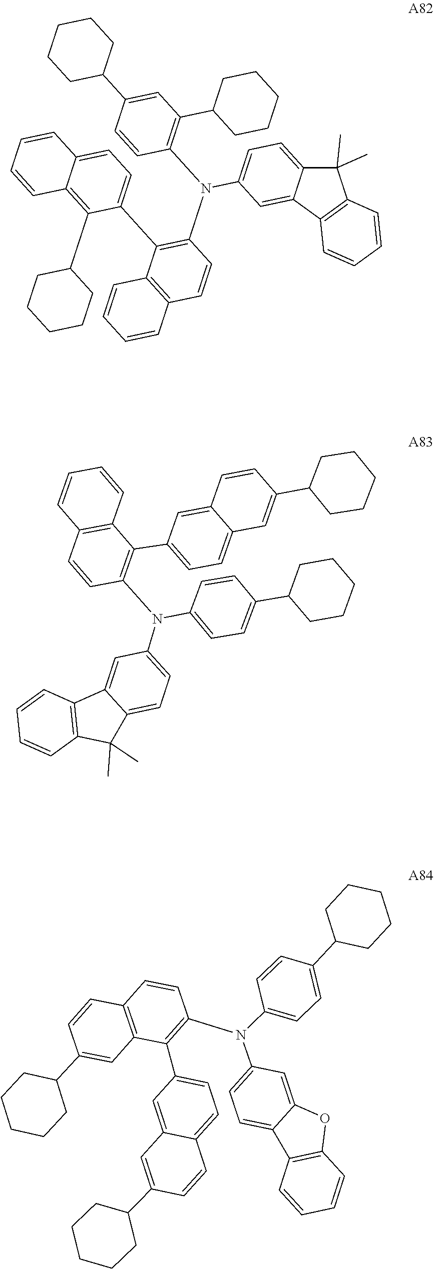 [SMILES: CC1(C)c2ccccc2-c2cc(N(c3ccc(C4CCCCC4)cc3C3CCCCC3)c3ccc4ccccc4c3-c3ccc4ccccc4c3C3CCCCC3)ccc21 ; CC1(C)c2ccccc2-c2cc(N(c3ccc(C4CCCCC4)cc3)c3ccc4ccccc4c3-c3ccc4cc(C5CCCCC5)ccc4c3)ccc21 ; c1ccc2c(c1)oc1cc(N(c3ccc(C4CCCCC4)cc3)c3ccc4ccc(C5CCCCC5)cc4c3-c3ccc4ccc(C5CCCCC5)cc4c3)ccc12]

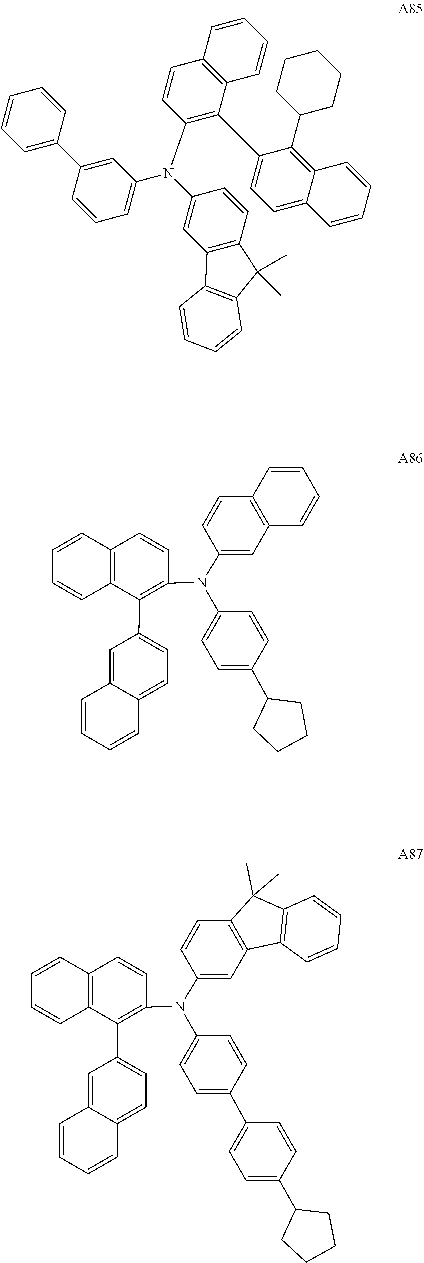 [SMILES: CC1(C)c2ccccc2-c2cc(N(c3cccc(-c4ccccc4)c3)c3ccc4ccccc4c3-c3ccc4ccccc4c3C3CCCCC3)ccc21 ; c1ccc2cc(-c3c(N(c4ccc(C5CCCC5)cc4)c4ccc5ccccc5c4)ccc4ccccc34)ccc2c1 ; CC1(C)c2ccccc2-c2cc(N(c3ccc(-c4ccc(C5CCCC5)cc4)cc3)c3ccc4ccccc4c3-c3ccc4ccccc4c3)ccc21]

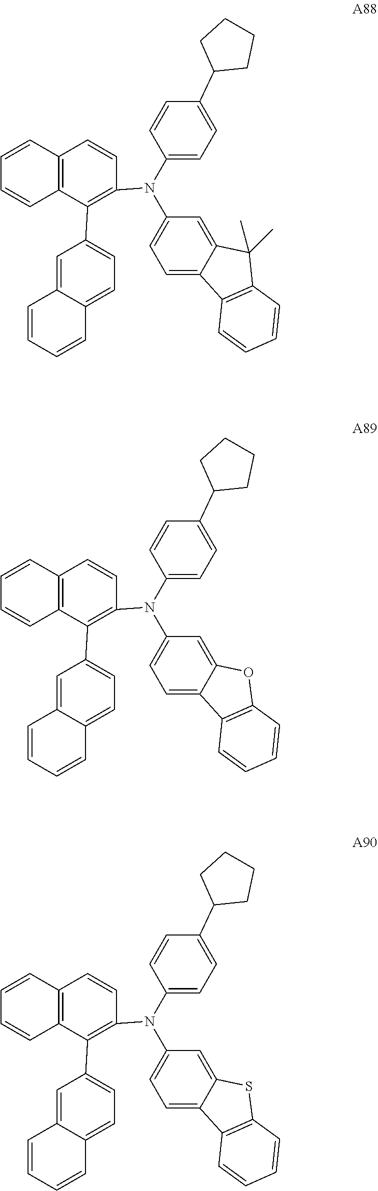 [SMILES: CC1(C)c2ccccc2-c2ccc(N(c3ccc(C4CCCC4)cc3)c3ccc4ccccc4c3-c3ccc4ccccc4c3)cc21 ; c1ccc2cc(-c3c(N(c4ccc(C5CCCC5)cc4)c4ccc5c(c4)oc4ccccc45)ccc4ccccc34)ccc2c1 ; c1ccc2cc(-c3c(N(c4ccc(C5CCCC5)cc4)c4ccc5c(c4)sc4ccccc45)ccc4ccccc34)ccc2c1]

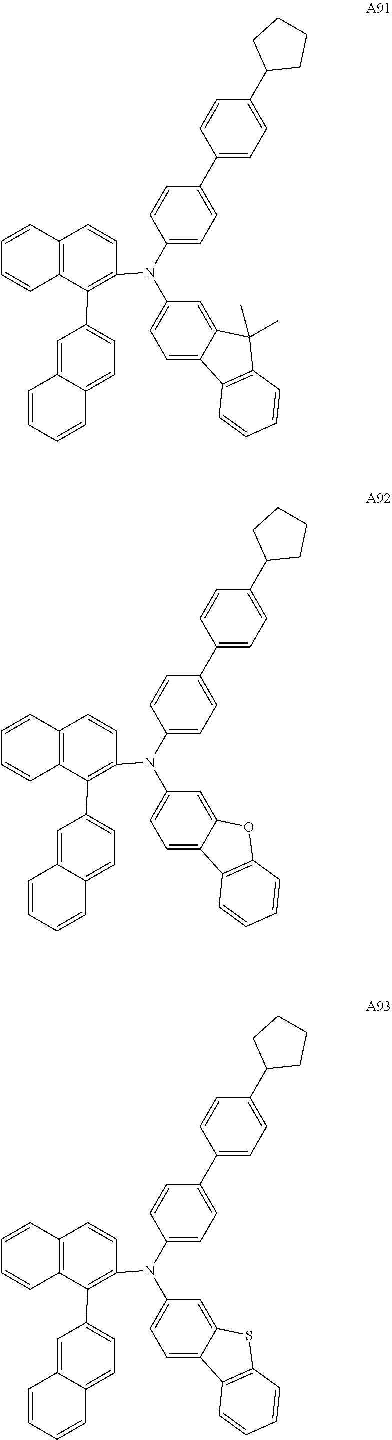 [SMILES: CC1(C)c2ccccc2-c2ccc(N(c3ccc(-c4ccc(C5CCCC5)cc4)cc3)c3ccc4ccccc4c3-c3ccc4ccccc4c3)cc21 ; c1ccc2cc(-c3c(N(c4ccc(-c5ccc(C6CCCC6)cc5)cc4)c4ccc5c(c4)oc4ccccc45)ccc4ccccc34)ccc2c1 ; c1ccc2cc(-c3c(N(c4ccc(-c5ccc(C6CCCC6)cc5)cc4)c4ccc5c(c4)sc4ccccc45)ccc4ccccc34)ccc2c1]

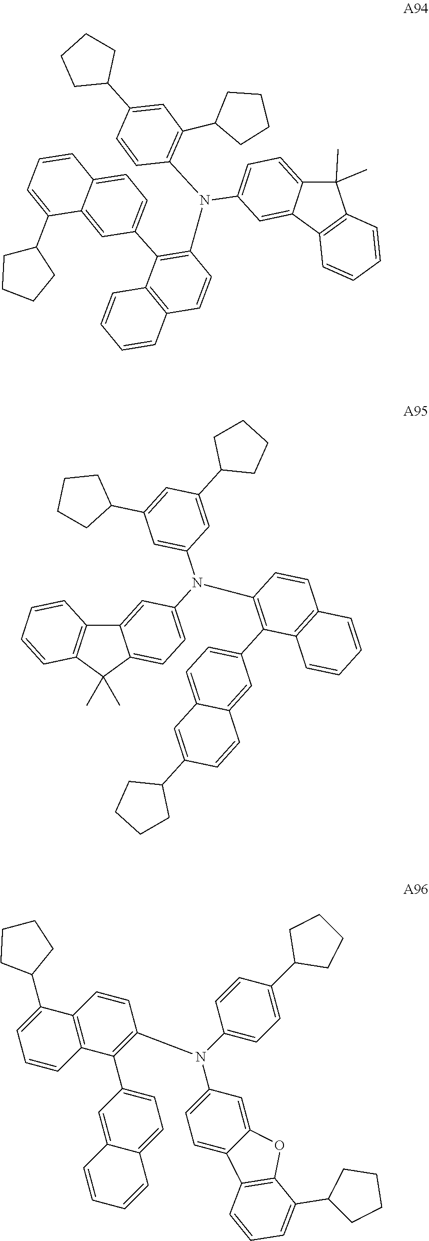 [SMILES: CC1(C)c2ccccc2-c2cc(N(c3ccc(C4CCCC4)cc3C3CCCC3)c3ccc4ccccc4c3-c3ccc4cccc(C5CCCC5)c4c3)ccc21 ; CC1(C)c2ccccc2-c2cc(N(c3cc(C4CCCC4)cc(C4CCCC4)c3)c3ccc4ccccc4c3-c3ccc4cc(C5CCCC5)ccc4c3)ccc21 ; c1ccc2cc(-c3c(N(c4ccc(C5CCCC5)cc4)c4ccc5c(c4)oc4c(C6CCCC6)cccc45)ccc4c(C5CCCC5)cccc34)ccc2c1]

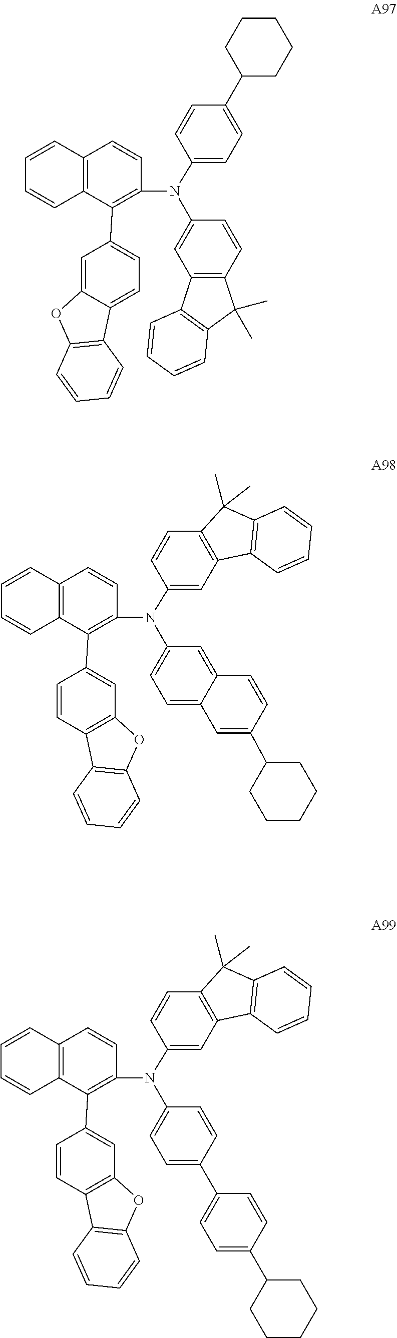 [SMILES: CC1(C)c2ccccc2-c2cc(N(c3ccc(C4CCCCC4)cc3)c3ccc4ccccc4c3-c3ccc4c(c3)oc3ccccc34)ccc21 ; CC1(C)c2ccccc2-c2cc(N(c3ccc4cc(C5CCCCC5)ccc4c3)c3ccc4ccccc4c3-c3ccc4c(c3)oc3ccccc34)ccc21 ; CC1(C)c2ccccc2-c2cc(N(c3ccc(-c4ccc(C5CCCCC5)cc4)cc3)c3ccc4ccccc4c3-c3ccc4c(c3)oc3ccccc34)ccc21]

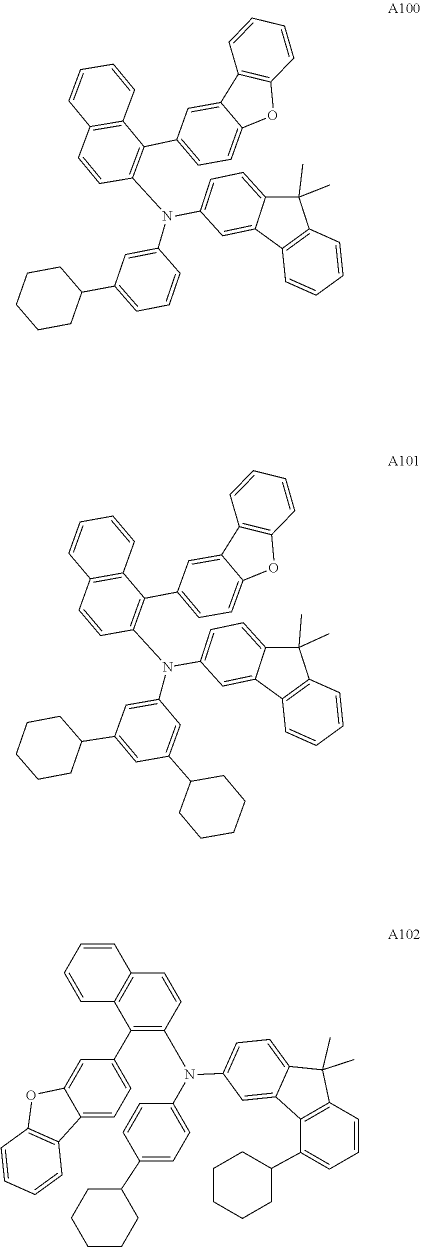 [SMILES: CC1(C)c2ccccc2-c2cc(N(c3cccc(C4CCCCC4)c3)c3ccc4ccccc4c3-c3ccc4oc5ccccc5c4c3)ccc21 ; CC1(C)c2ccccc2-c2cc(N(c3cc(C4CCCCC4)cc(C4CCCCC4)c3)c3ccc4ccccc4c3-c3ccc4oc5ccccc5c4c3)ccc21 ; CC1(C)c2ccc(N(c3ccc(C4CCCCC4)cc3)c3ccc4ccccc4c3-c3ccc4c(c3)oc3ccccc34)cc2-c2c(C3CCCCC3)cccc21]

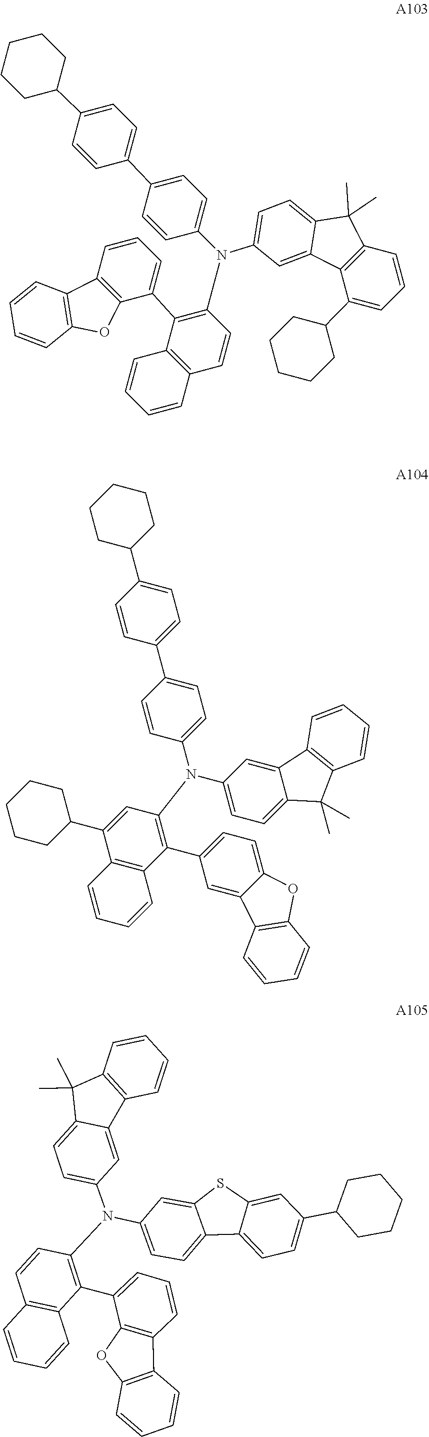 [SMILES: CC1(C)c2ccc(N(c3ccc(-c4ccc(C5CCCCC5)cc4)cc3)c3ccc4ccccc4c3-c3cccc4c3oc3ccccc34)cc2-c2c(C3CCCCC3)cccc21 ; CC1(C)c2ccccc2-c2cc(N(c3ccc(-c4ccc(C5CCCCC5)cc4)cc3)c3cc(C4CCCCC4)c4ccccc4c3-c3ccc4oc5ccccc5c4c3)ccc21 ; CC1(C)c2ccccc2-c2cc(N(c3ccc4c(c3)sc3cc(C5CCCCC5)ccc34)c3ccc4ccccc4c3-c3cccc4c3oc3ccccc34)ccc21]

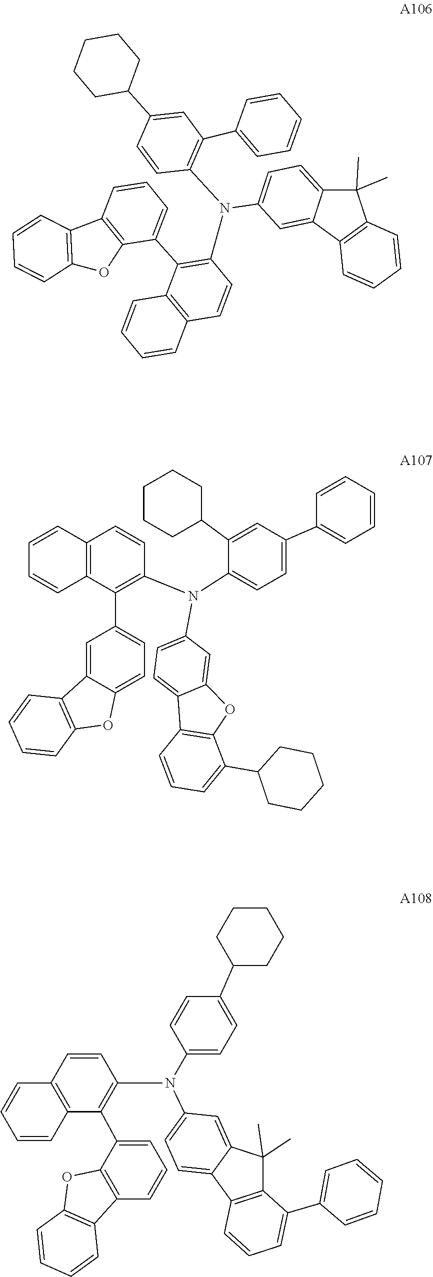 [SMILES: CC1(C)c2ccccc2-c2cc(N(c3ccc(C4CCCCC4)cc3-c3ccccc3)c3ccc4ccccc4c3-c3cccc4c3oc3ccccc34)ccc21 ; c1ccc(-c2ccc(N(c3ccc4c(c3)oc3c(C5CCCCC5)cccc34)c3ccc4ccccc4c3-c3ccc4oc5ccccc5c4c3)c(C3CCCCC3)c2)cc1 ; CC1(C)c2cc(N(c3ccc(C4CCCCC4)cc3)c3ccc4ccccc4c3-c3cccc4c3oc3ccccc34)ccc2-c2cccc(-c3ccccc3)c21]

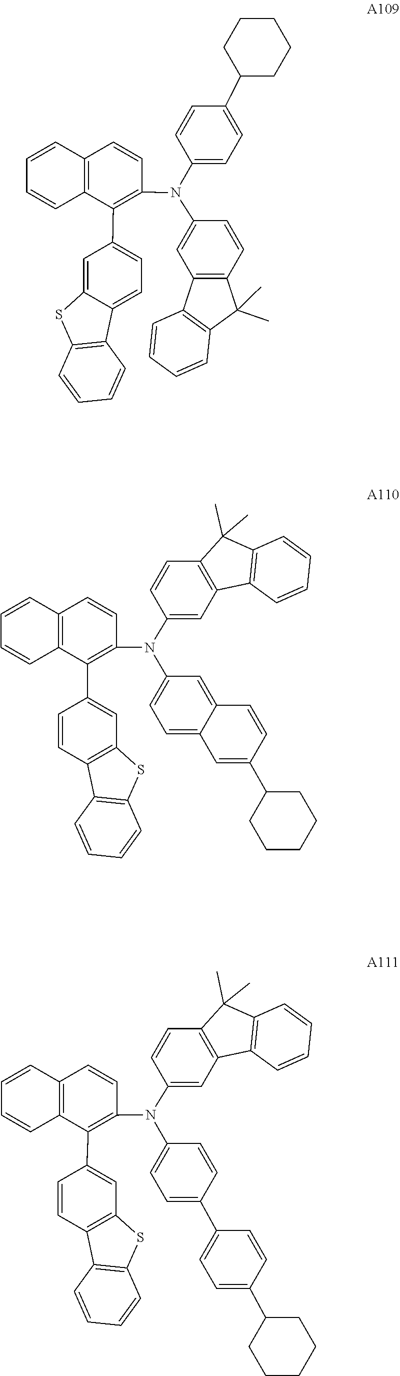 [SMILES: CC1(C)c2ccccc2-c2cc(N(c3ccc(C4CCCCC4)cc3)c3ccc4ccccc4c3-c3ccc4c(c3)sc3ccccc34)ccc21 ; CC1(C)c2ccccc2-c2cc(N(c3ccc4cc(C5CCCCC5)ccc4c3)c3ccc4ccccc4c3-c3ccc4c(c3)sc3ccccc34)ccc21 ; CC1(C)c2ccccc2-c2cc(N(c3ccc(-c4ccc(C5CCCCC5)cc4)cc3)c3ccc4ccccc4c3-c3ccc4c(c3)sc3ccccc34)ccc21]

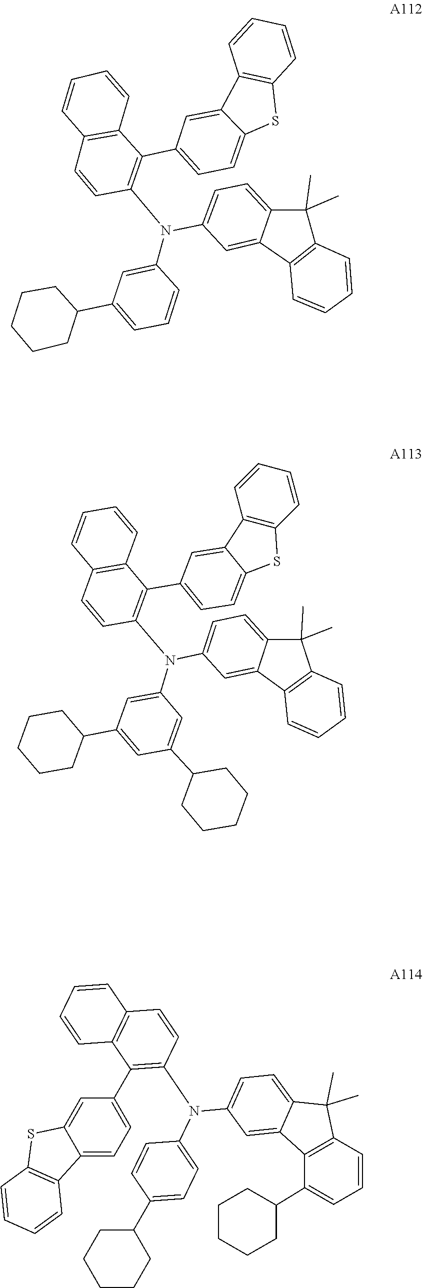 [SMILES: CC1(C)c2ccccc2-c2cc(N(c3cccc(C4CCCCC4)c3)c3ccc4ccccc4c3-c3ccc4sc5ccccc5c4c3)ccc21 ; CC1(C)c2ccccc2-c2cc(N(c3cc(C4CCCCC4)cc(C4CCCCC4)c3)c3ccc4ccccc4c3-c3ccc4sc5ccccc5c4c3)ccc21 ; CC1(C)c2ccc(N(c3ccc(C4CCCCC4)cc3)c3ccc4ccccc4c3-c3ccc4c(c3)sc3ccccc34)cc2-c2c(C3CCCCC3)cccc21]

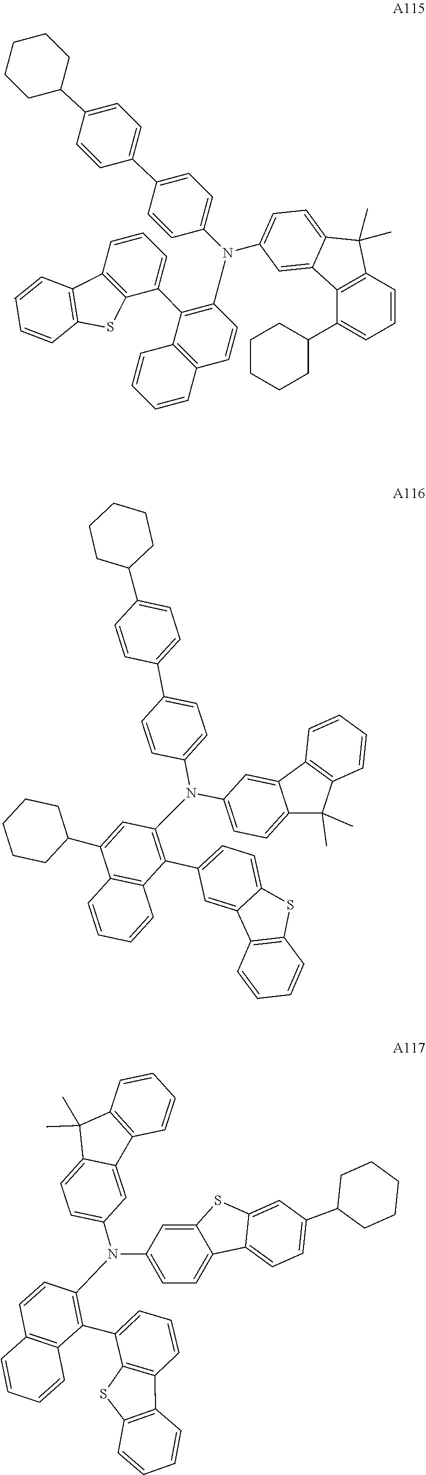 [SMILES: Cc1ccc2sc3c(-c4c(N(c5ccc(-c6ccc(C7CCCCC7)cc6)cc5)c5ccc6c(c5)-c5c(C7CCCCC7)cccc5C6(C)C)ccc5ccccc45)cccc3c2c1 ; CC1(C)c2ccccc2-c2cc(N(c3ccc(-c4ccc(C5CCCCC5)cc4)cc3)c3cc(C4CCCCC4)c4ccccc4c3-c3ccc4sc5ccccc5c4c3)ccc21 ; CC1(C)c2ccccc2-c2cc(N(c3ccc4c(c3)sc3cc(C5CCCCC5)ccc34)c3ccc4ccccc4c3-c3cccc4c3sc3ccccc34)ccc21]

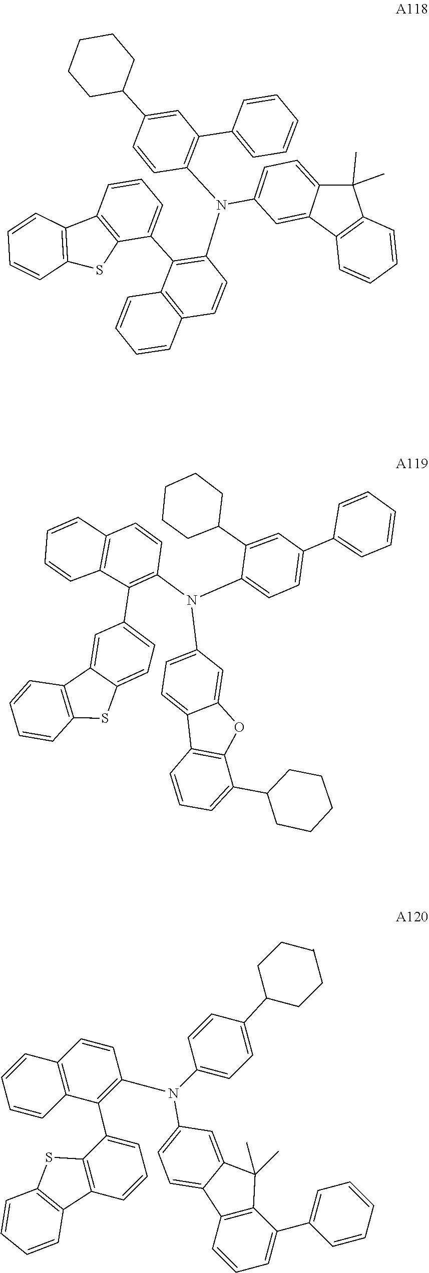 [SMILES: CC1(C)c2ccccc2-c2cc(N(c3ccc(C4CCCCC4)cc3-c3ccccc3)c3ccc4ccccc4c3-c3cccc4c3sc3ccccc34)ccc21 ; c1ccc(-c2ccc(N(c3ccc4c(c3)oc3c(C5CCCCC5)cccc34)c3ccc4ccccc4c3-c3ccc4sc5ccccc5c4c3)c(C3CCCCC3)c2)cc1 ; CC1CCC(c2ccc(N(c3ccc4c(c3)C(C)(C)c3c(-c5ccccc5)cccc3-4)c3ccc4ccccc4c3-c3cccc4c3sc3ccccc34)cc2)CC1]

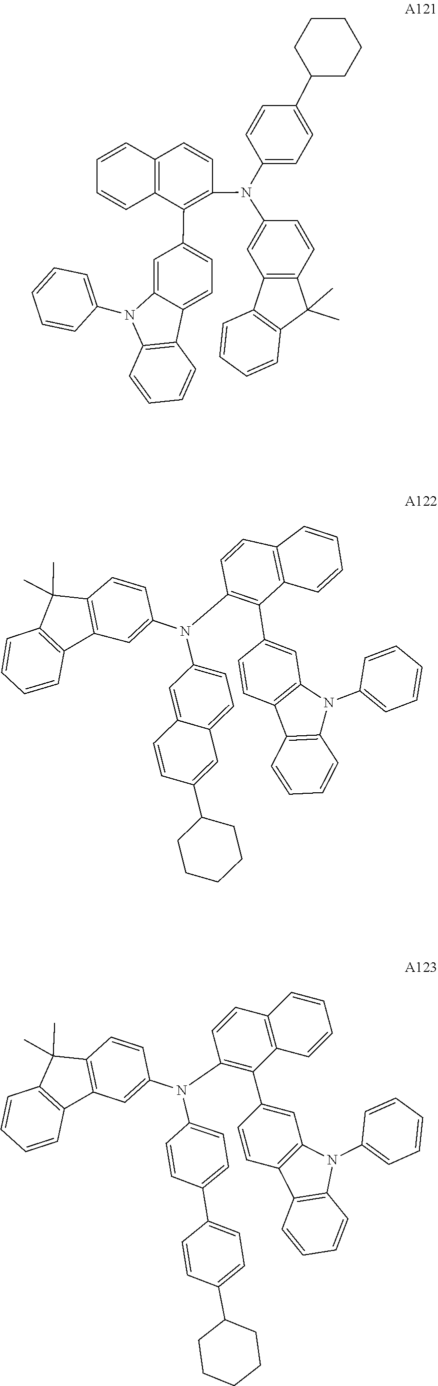 [SMILES: CC1(C)c2ccccc2-c2cc(N(c3ccc(C4CCCCC4)cc3)c3ccc4ccccc4c3-c3ccc4c5ccccc5n(-c5ccccc5)c4c3)ccc21 ; CC1(C)c2ccccc2-c2cc(N(c3ccc4cc(C5CCCCC5)ccc4c3)c3ccc4ccccc4c3-c3ccc4c5ccccc5n(-c5ccccc5)c4c3)ccc21 ; CC1(C)c2ccccc2-c2cc(N(c3ccc(-c4ccc(C5CCCCC5)cc4)cc3)c3ccc4ccccc4c3-c3ccc4c5ccccc5n(-c5ccccc5)c4c3)ccc21]

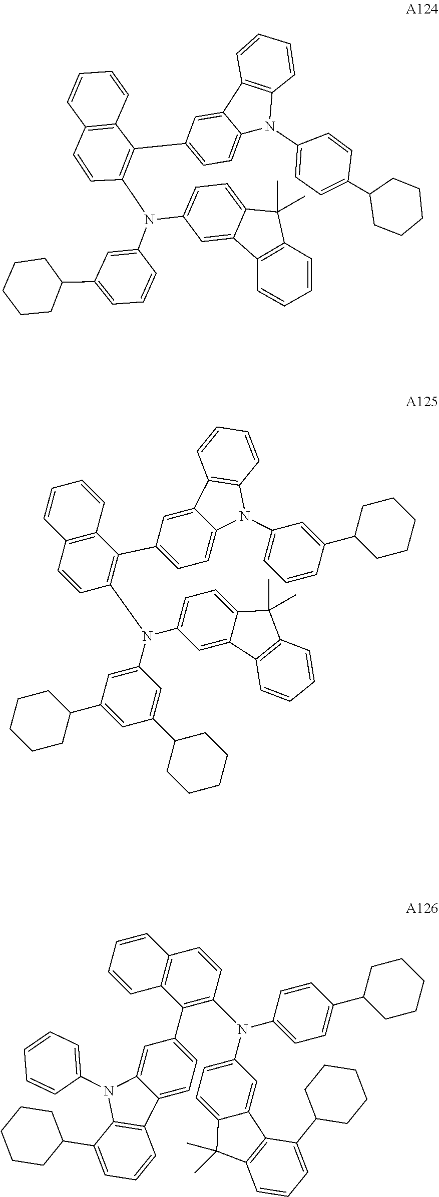 [SMILES: CC1(C)c2ccccc2-c2cc(N(c3cccc(C4CCCCC4)c3)c3ccc4ccccc4c3-c3ccc4c(c3)c3ccccc3n4-c3ccc(C4CCCCC4)cc3)ccc21 ; CC1(C)c2ccccc2-c2cc(N(c3cc(C4CCCCC4)cc(C4CCCCC4)c3)c3ccc4ccccc4c3-c3ccc4c(c3)c3ccccc3n4-c3cccc(C4CCCCC4)c3)ccc21 ; CC1(C)c2ccc(N(c3ccc(C4CCCCC4)cc3)c3ccc4ccccc4c3-c3ccc4c5cccc(C6CCCCC6)c5n(-c5ccccc5)c4c3)cc2-c2c(C3CCCCC3)cccc21]

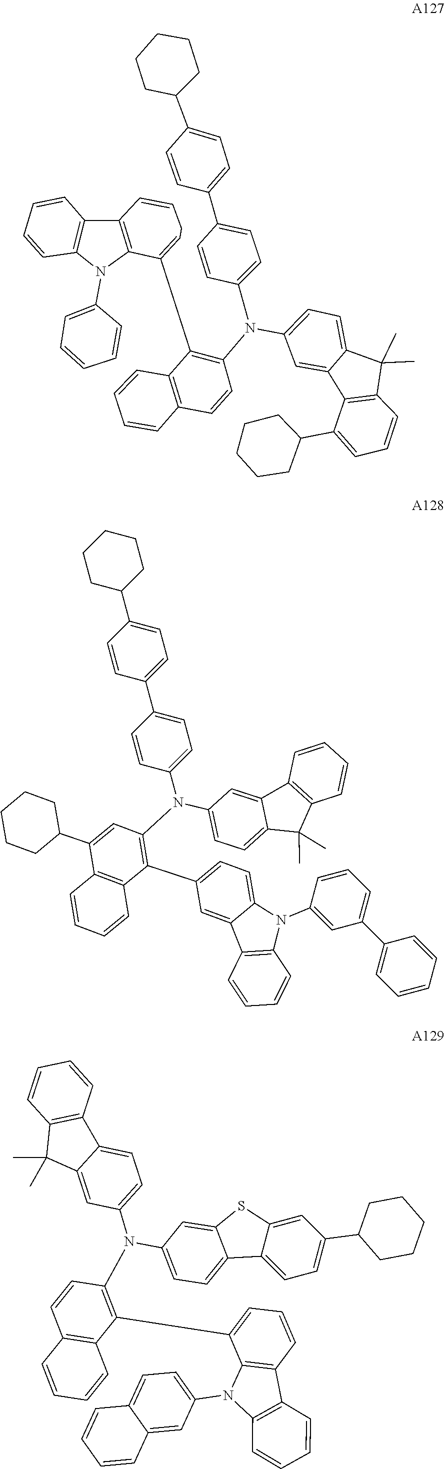

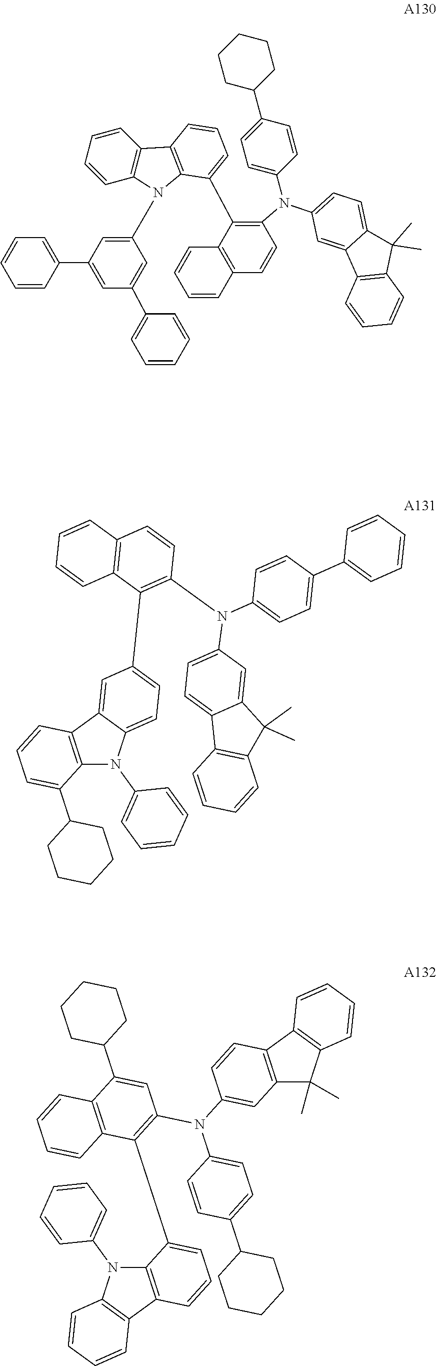 [SMILES: CC1(C)c2ccccc2-c2cc(N(c3ccc(C4CCCCC4)cc3)c3ccc4ccccc4c3-c3cccc4c5ccccc5n(-c5cc(-c6ccccc6)cc(-c6ccccc6)c5)c34)ccc21 ; CC1(C)c2ccccc2-c2ccc(N(c3ccc(-c4ccccc4)cc3)c3ccc4ccccc4c3-c3ccc4c(c3)c3cccc(C5CCCCC5)c3n4-c3ccccc3)cc21 ; CC1(C)c2ccccc2-c2ccc(N(c3ccc(C4CCCCC4)cc3)c3cc(C4CCCCC4)c4ccccc4c3-c3cccc4c5ccccc5n(-c5ccccc5)c34)cc21]

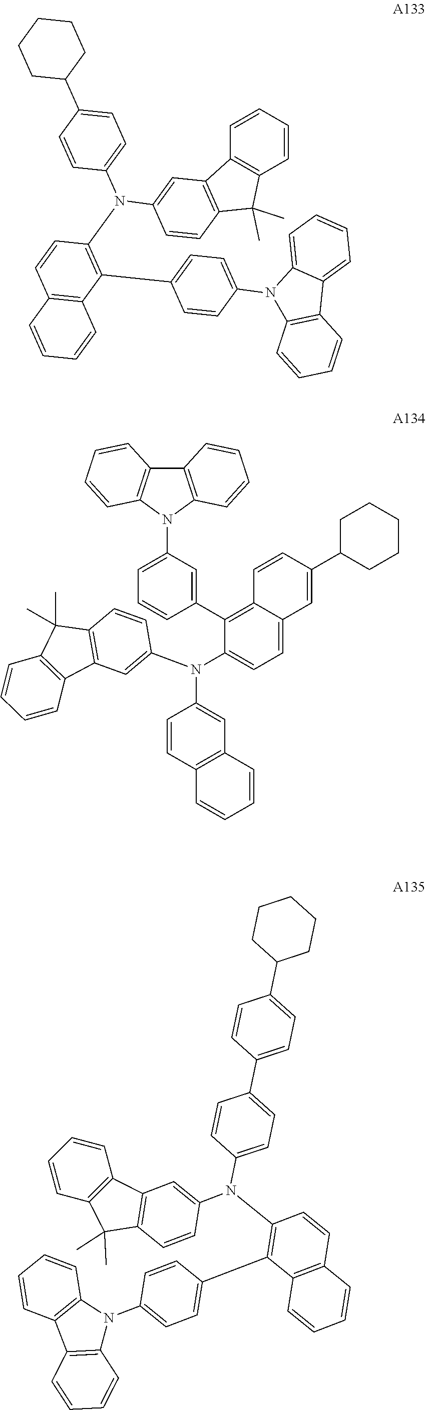 [SMILES: CC1(C)c2ccccc2-c2cc(N(c3ccc(C4CCCCC4)cc3)c3ccc4ccccc4c3-c3ccc(-n4c5ccccc5c5ccccc54)cc3)ccc21 ; CC1(C)c2ccccc2-c2cc(N(c3ccc4ccccc4c3)c3ccc4cc(C5CCCCC5)ccc4c3-c3cccc(-n4c5ccccc5c5ccccc54)c3)ccc21 ; CC1(C)c2ccccc2-c2cc(N(c3ccc(-c4ccc(C5CCCCC5)cc4)cc3)c3ccc4ccccc4c3-c3ccc(-n4c5ccccc5c5ccccc54)cc3)ccc21]

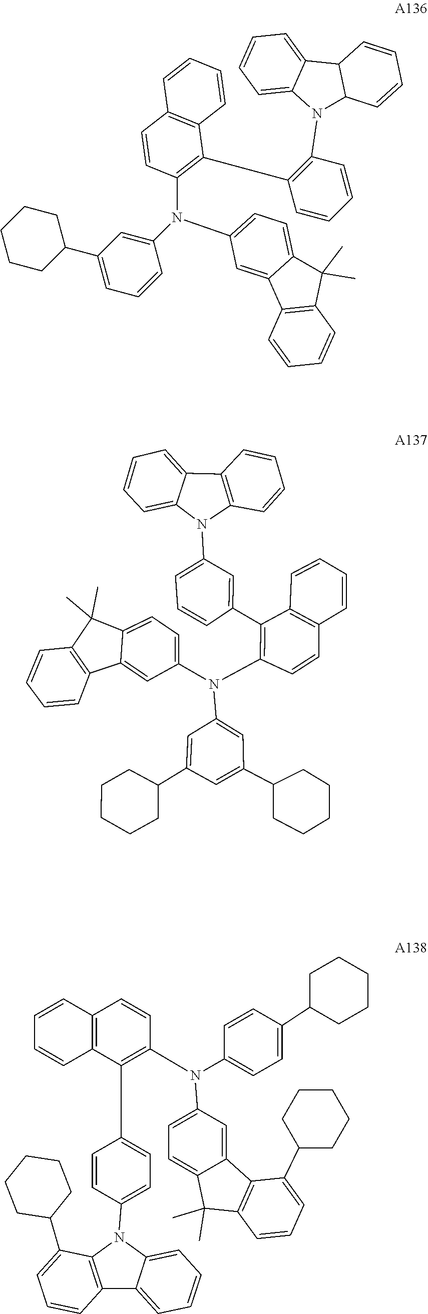 [SMILES: CC1(C)c2ccccc2-c2cc(N(c3cccc(C4CCCCC4)c3)c3ccc4ccccc4c3-c3ccccc3N3c4ccccc4C4C=CC=CC43)ccc21 ; CC1(C)c2ccccc2-c2cc(N(c3cc(C4CCCCC4)cc(C4CCCCC4)c3)c3ccc4ccccc4c3-c3cccc(-n4c5ccccc5c5ccccc54)c3)ccc21 ; CC1(C)c2ccc(N(c3ccc(C4CCCCC4)cc3)c3ccc4ccccc4c3-c3ccc(-n4c5ccccc5c5cccc(C6CCCCC6)c54)cc3)cc2-c2c(C3CCCCC3)cccc21]

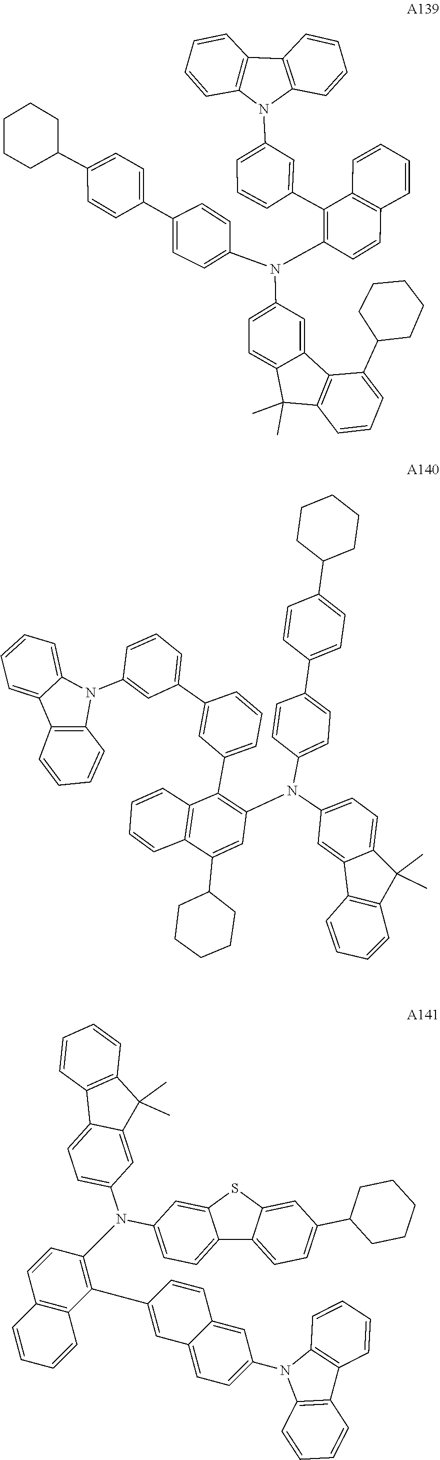 [SMILES: CC1(C)c2ccc(N(c3ccc(-c4ccc(C5CCCCC5)cc4)cc3)c3ccc4ccccc4c3-c3cccc(-n4c5ccccc5c5ccccc54)c3)cc2-c2c(C3CCCCC3)cccc21 ; CC1(C)c2ccccc2-c2cc(N(c3ccc(-c4ccc(C5CCCCC5)cc4)cc3)c3cc(C4CCCCC4)c4ccccc4c3-c3cccc(-c4cccc(-n5c6ccccc6c6ccccc65)c4)c3)ccc21 ; CC1(C)c2ccccc2-c2ccc(N(c3ccc4c(c3)sc3cc(C5CCCCC5)ccc34)c3ccc4ccccc4c3-c3ccc4cc(-n5c6ccccc6c6ccccc65)ccc4c3)cc21]

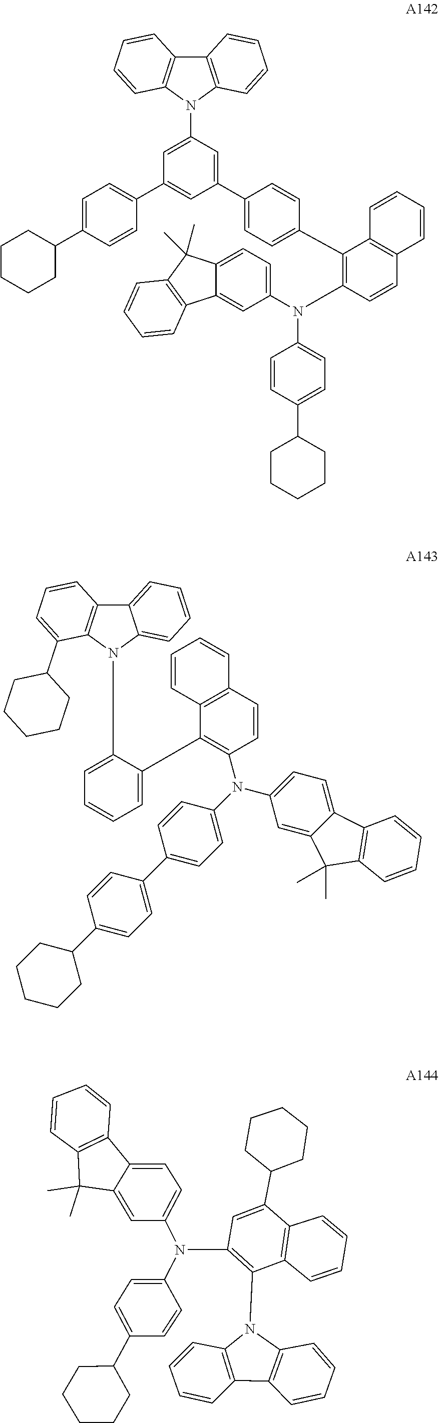 [SMILES: CC1(C)c2ccccc2-c2cc(N(c3ccc(C4CCCCC4)cc3)c3ccc4ccccc4c3-c3ccc(-c4cc(-c5ccc(C6CCCCC6)cc5)cc(-n5c6ccccc6c6ccccc65)c4)cc3)ccc21 ; CC1(C)c2ccccc2-c2ccc(N(c3ccc(-c4ccc(C5CCCCC5)cc4)cc3)c3ccc4ccccc4c3-c3ccccc3-n3c4ccccc4c4cccc(C5CCCCC5)c43)cc21 ; CC1(C)c2ccccc2-c2ccc(N(c3ccc(C4CCCCC4)cc3)c3cc(C4CCCCC4)c4ccccc4c3-n3c4ccccc4c4ccccc43)cc21]

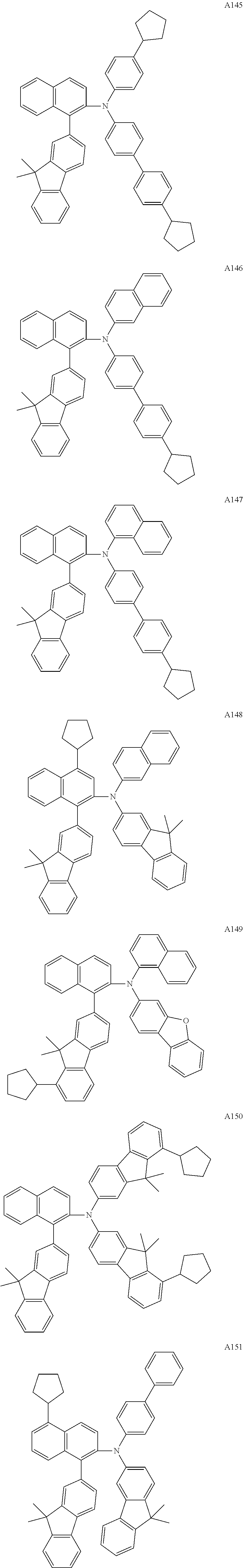

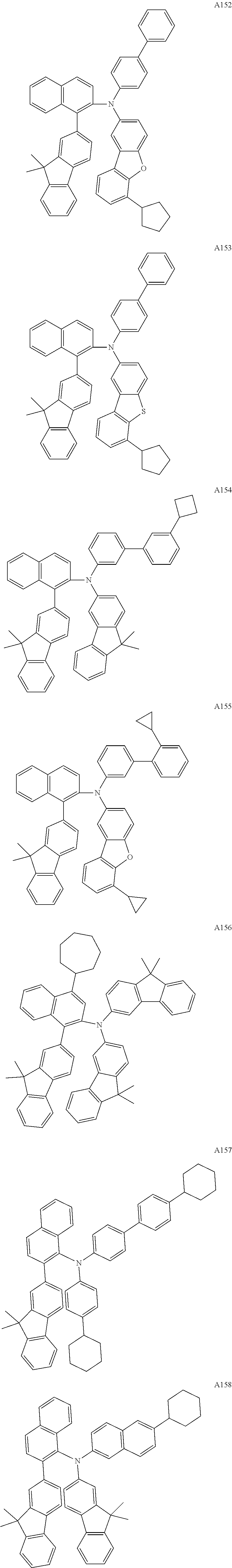

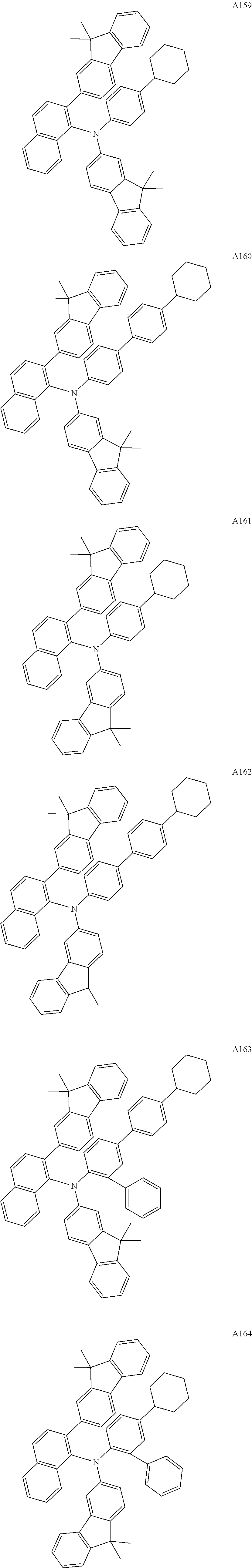

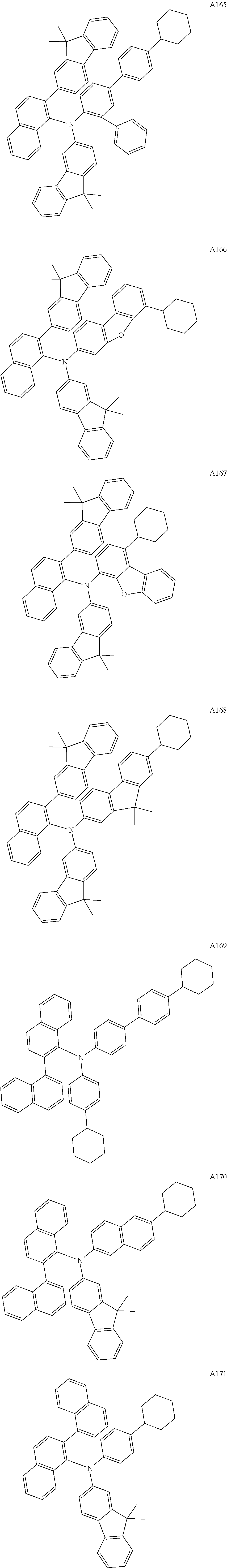

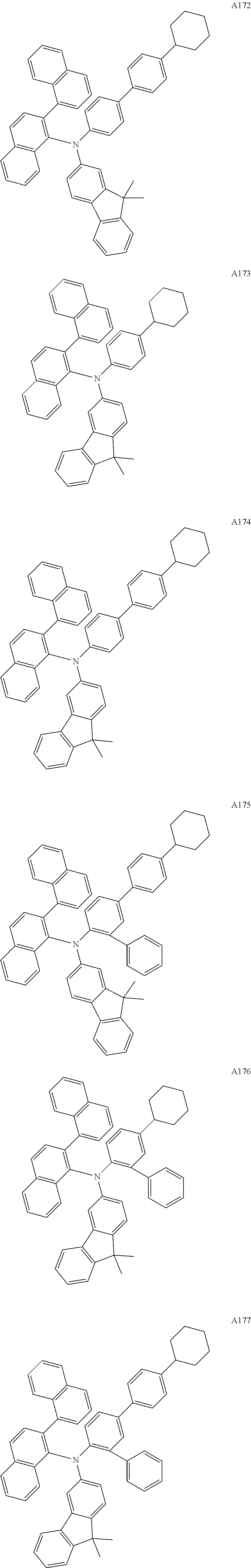

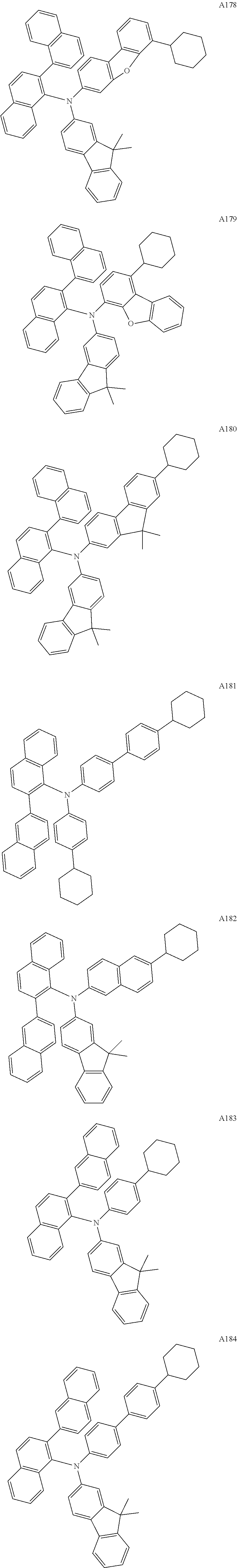

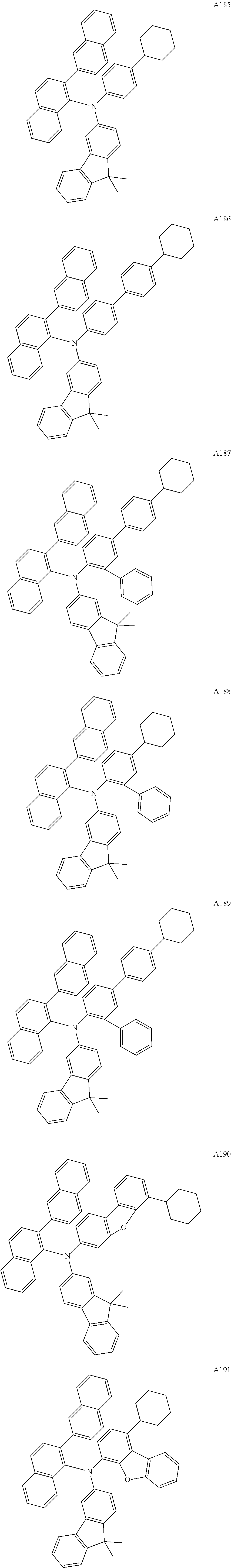

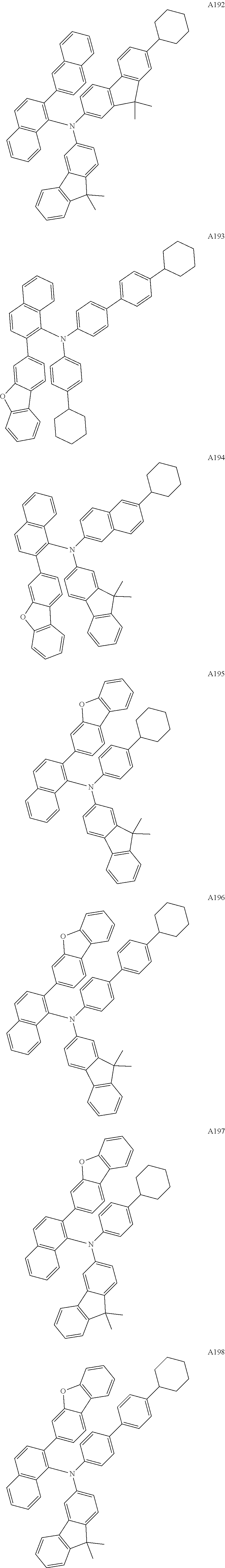

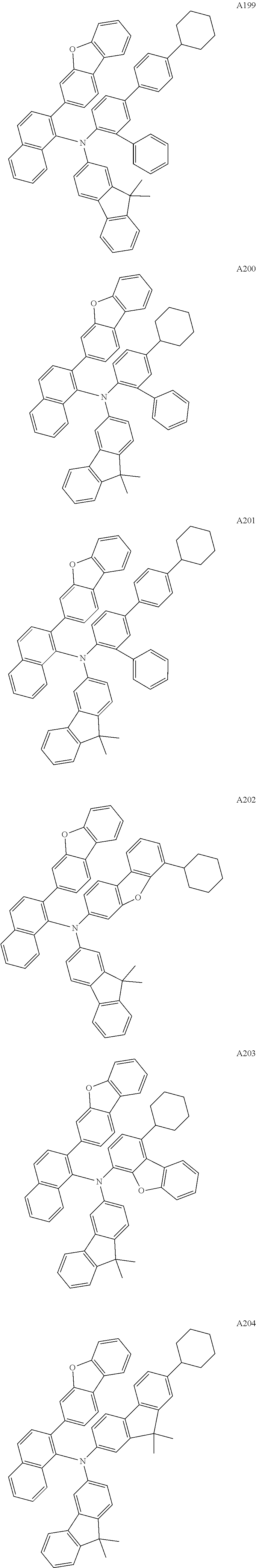

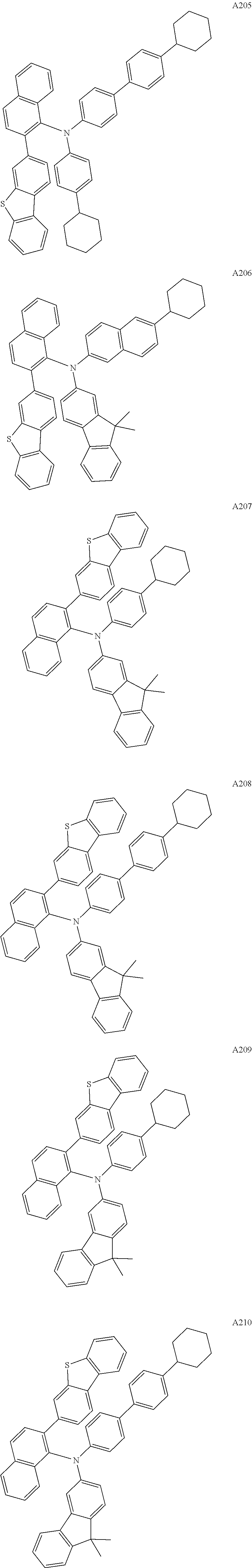

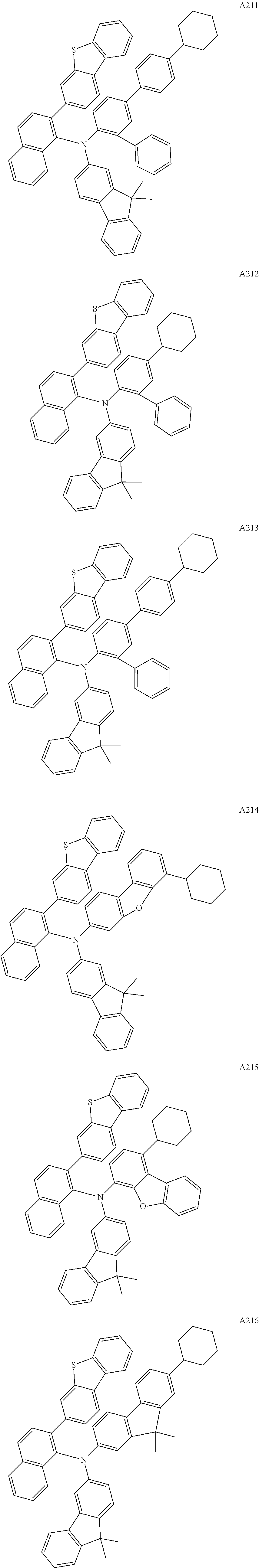

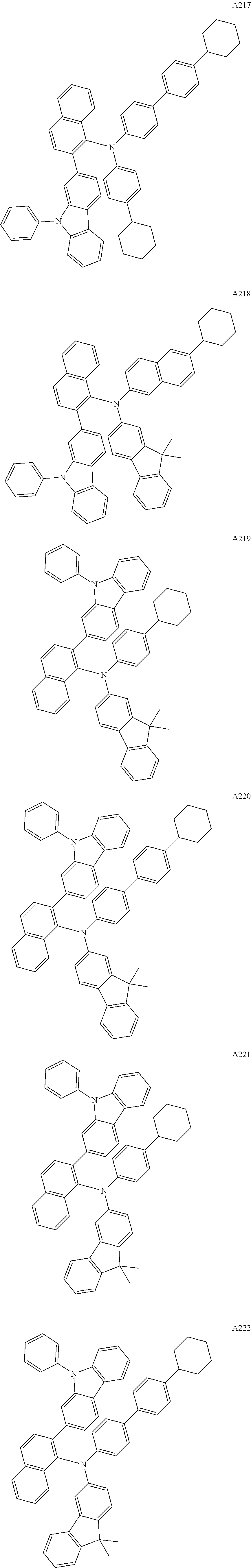

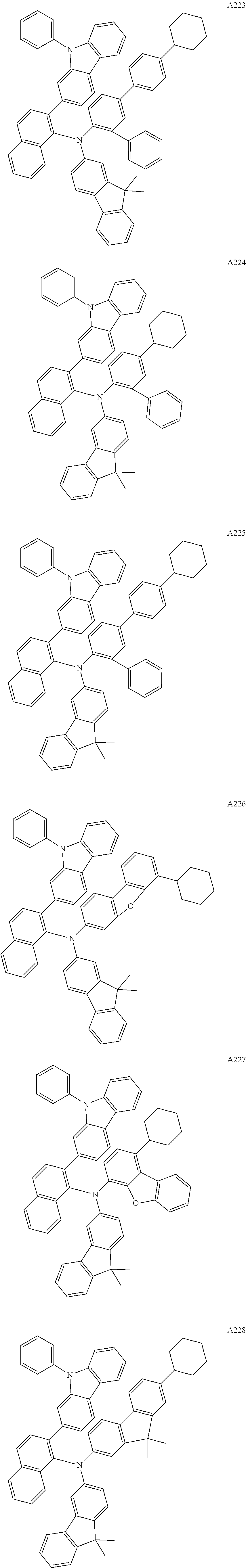

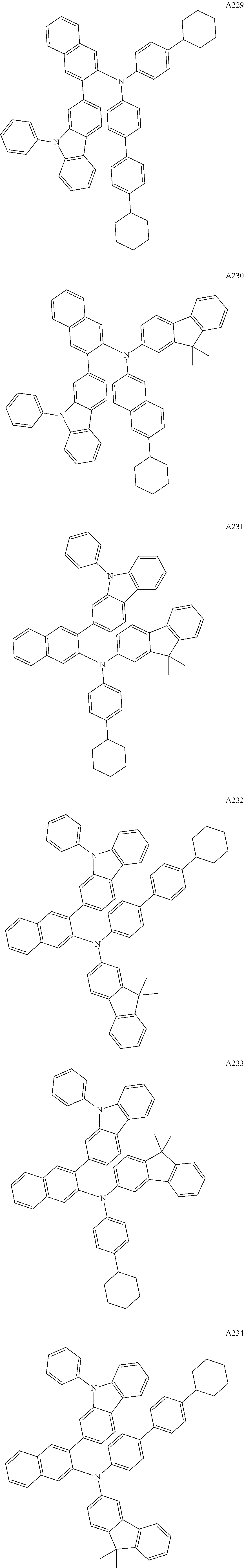

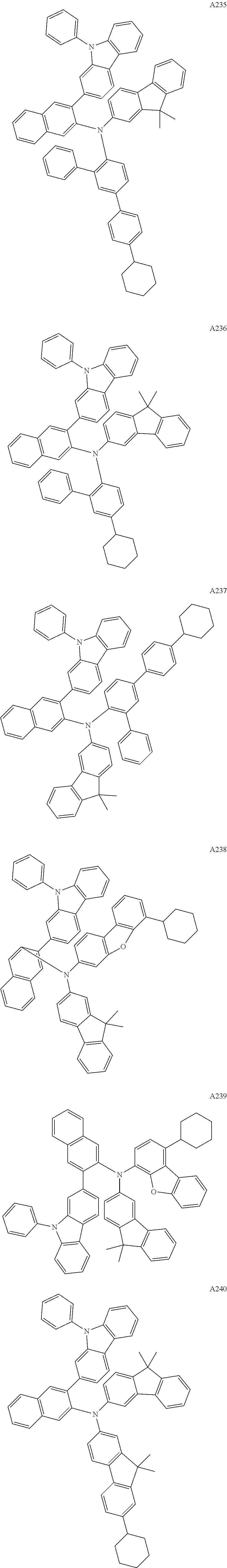

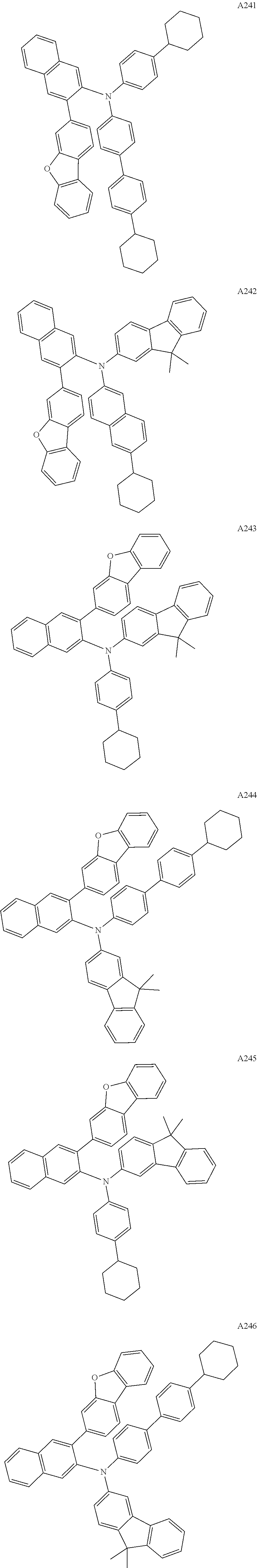

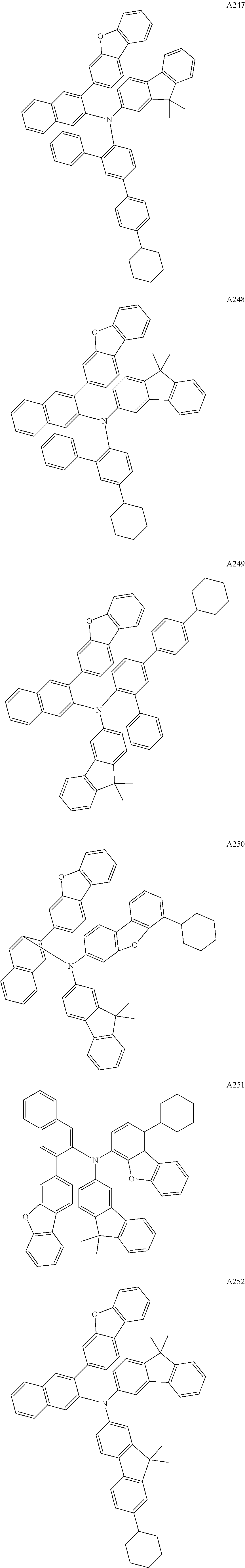

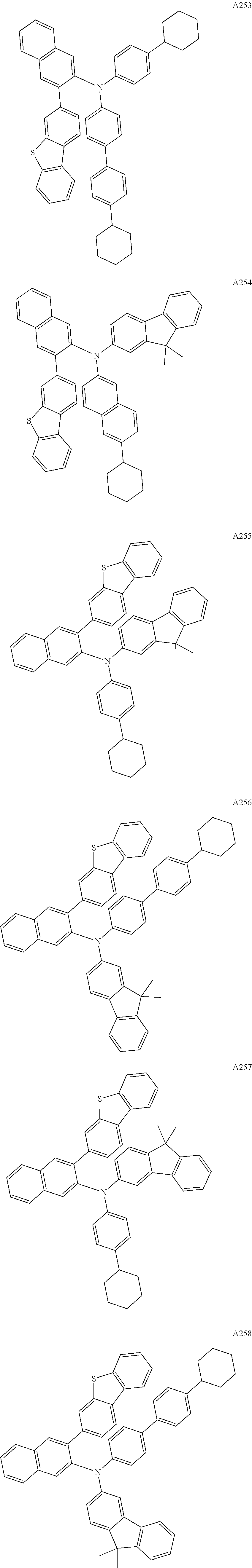

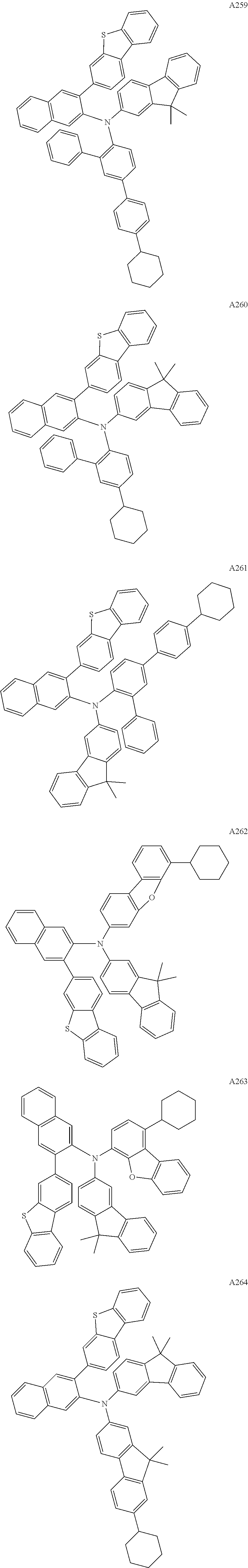

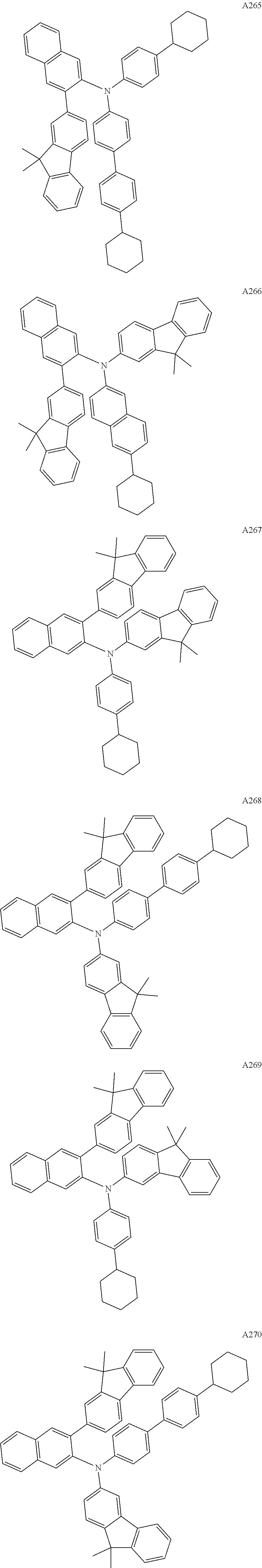

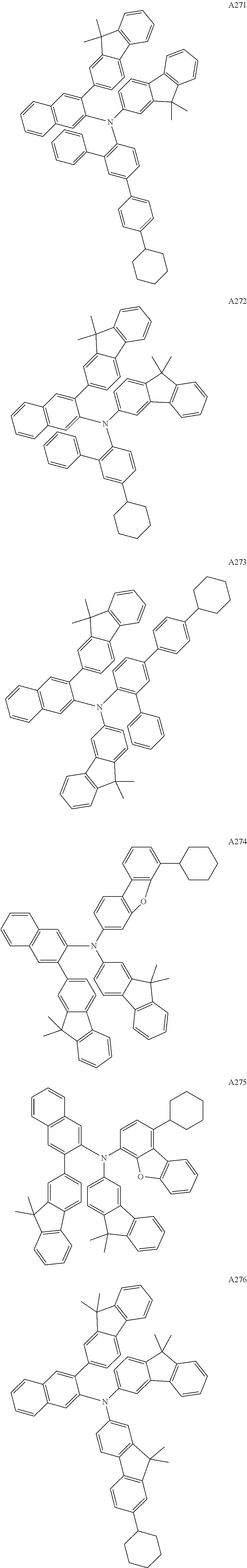

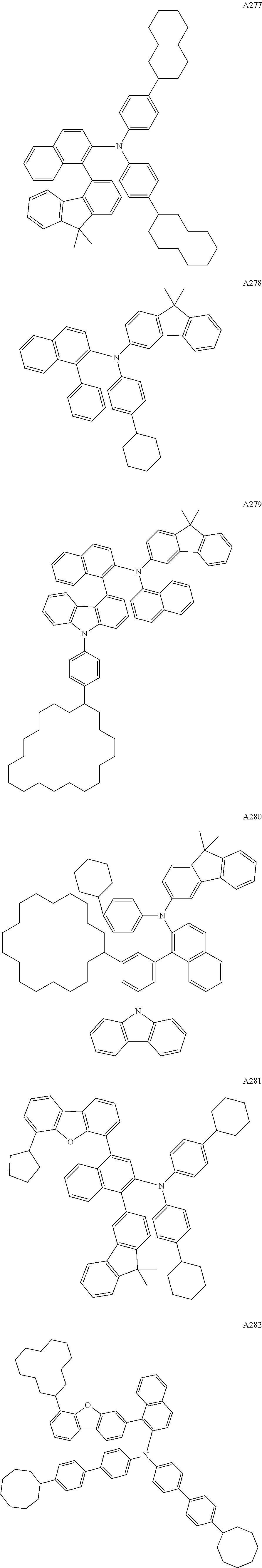

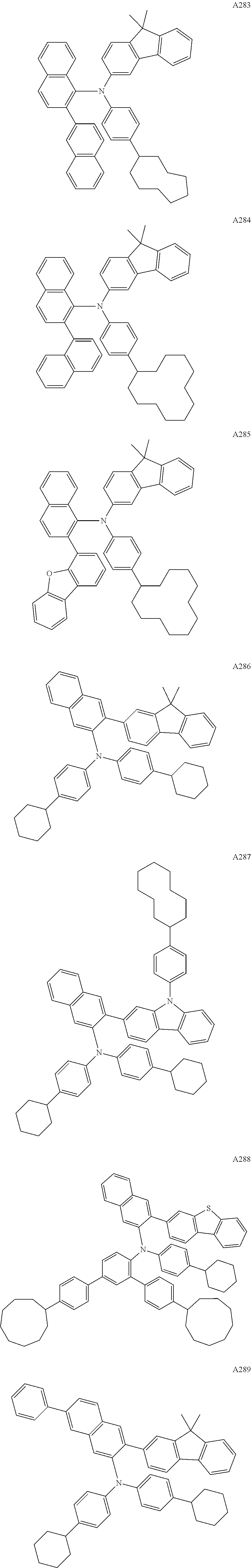

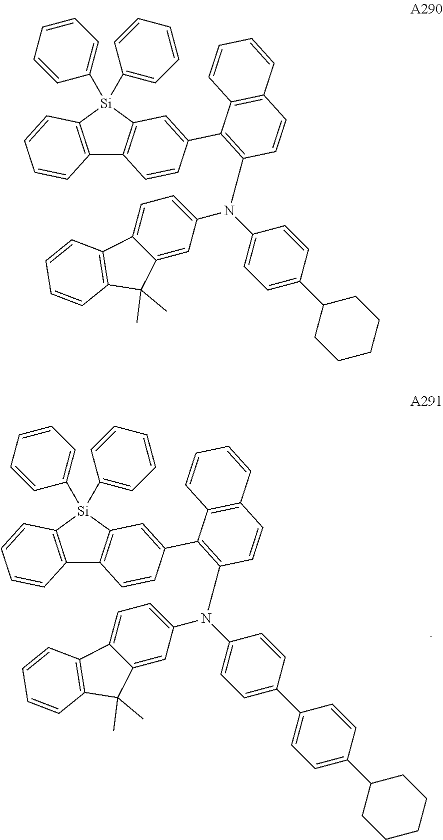 [SMILES: CC1(C)c2ccccc2-c2ccc(N(c3ccc(C4CCCCC4)cc3)c3ccc4ccccc4c3-c3ccc4c(c3)[Si](c3ccccc3)(c3ccccc3)c3ccccc3-4)cc21 ; CC1(C)c2ccccc2-c2ccc(N(c3ccc(-c4ccc(C5CCCCC5)cc4)cc3)c3ccc4ccccc4c3-c3ccc4c(c3)[Si](c3ccccc3)(c3ccccc3)c3ccccc3-4)cc21]

Preferably, the organic layer includes a light emitting layer and an electron blocking layer.

Preferably, the light emitting layer contains the compound I, and the electron blocking layer contains the compound II.

Preferably, the light emitting layer contains the compound II, and the electron blocking layer contains the compound I.

In a preferred technical solution of the disclosure, one of the compound I and compounds II is used as the light emitting layer material and the other is used as the electron blocking layer material, due to both of two materials have hole transporting properties, and can balance the effect of rapid electron transferring when they are used as materials of the electron blocking layer and the light emitting layer, respectively, so that the composite center is located in the center of the light emitting layer, which can further improve the device efficiency.

Preferably, the light emitting layer contains a first host material, a second host material and a doping material.

The first host material is the compound I, the electron blocking layer contains the compound II, or, the first host material is the compound II, and the electron blocking layer contains the compound I.

More further, the disclosure preferably uses compound I or compound II as one of the host materials in the dual host light emitting layer, which can better adjust the distribution of carriers inside the light emitting layer, so as to regulate the carrier composite region, and also has a higher spatial packing structure. As one of the dual hosts, it can improve the light extraction efficiency, and can further improve the efficiency in low gray-scale by cooperating with other host materials, reduce the roll-off degree of device efficiency, thereby further improving the device efficiency.

In a preferred embodiment, the mass ratio of the first host material to the second host material is 0.01:1-1.5:1, for example, 0.05:1, 0.1:1, 0.15:1, 0.2:1, 0.25:1, 0.3:1, 0.35:1, 0.4:1, 0.45:1, 0.5:1, 0.55:1, 0.6.1, 0.65:1, 0.7:1, 0.75:1, 0.8:1, 0.85:1, 0.9:1, 0.95:1, 1:1, 1.05:1, 1.1:1, 1.15:1, 1.2:1, 1.25:1, 1.3:1, 1.35:1, 1.4:1, 1.45:1, etc., preferably 0.1:1-1:1.

In yet another preferred technical solution of the disclosure, the addition amount of compound I or compound II in the dual host light emitting layer is selected within the above mentioned specific range, within which the efficiency of the device can be further improved. If the addition amount is too high, the hole transporting will be too fast, which in turn will damage the internal balance of the device. If the addition amount is too low, the effect of regulation will not be achieved.

Preferably, the second host material includes a phosphorescent material.

Preferably, the thickness of the above mentioned dual host light emitting layer is 10-60 nm, for example, 15 nm, 20 nm, 25 nm, 30 nm, 35 nm, 40 nm, 45 nm, 50 nm, 55 nm, etc., preferably 20-50 nm.

Preferably, the thickness of the light emitting layer containing compound I or compound II in the disclosure is 10-60 nm. Within this thickness range, the efficiency of the device can be further improved. If the thickness is too small, the internal excitons cannot be fully recombined. If the thickness is too large, the hole and electron transporting process will be longer, and the internal loss will become more, which will reduce the efficiency.

Preferably, the thickness of the electron blocking layer is 2-100 nm, for example, 10 nm, 20 nm, 30 nm, 40 nm, 50 nm, 60 nm, 70 nm, 80 nm, 90 nm, etc., preferably 3-90 nm.

Preferably, the thickness of the electron blocking layer containing compound I or compound II in the disclosure is 2-100 nm. Within this thickness range, the efficiency of the device can be further improved. If the thickness is too small, the internal excitons cannot be fully recombined. If the thickness is too large, the hole and electron transporting process will be longer, and the internal loss will become more, which will reduce the efficiency.

Preferably, the organic layer also includes a hole injection layer, a hole transporting layer, an electron transporting layer and an electron injection layer.

Preferably, in compound I, the Ar¹ is selected from the group consisting of any of the following substituted or unsubstituted groups: phenyl, biphenyl, terphenyl, naphthyl, phenanthryl, anthracyl, dibenzofuranyl, dibenzothiophenyl, carbazolyl, 9,9dimethylfluorenyl, 9,9diphenylfluorenyl, spirofluorenyl, triphenylene, fluoranthenyl, benzo9,9dimethylfluorenyl, and benzospirofluorenyl.

Preferably, in compound I, the Ar² is selected from the group consisting of any of the following substituted or unsubstituted groups: phenyl, biphenyl, terphenyl, naphthyl, phenanthryl, anthracyl, dibenzofuranyl, dibenzothiophenyl, carbazolyl, 9,9dimethylfluorenyl, 9,9diphenylfluorenyl, spirofluorenyl, triphenylene, fluoranthenyl, benzo9,9dimethylfluorenyl, and benzospirofluorenyl, preferably a substituted or unsubstituted naphthyl.

Preferably, the Ar² is selected from the group consisting of any of the following substituted or unsubstituted groups:

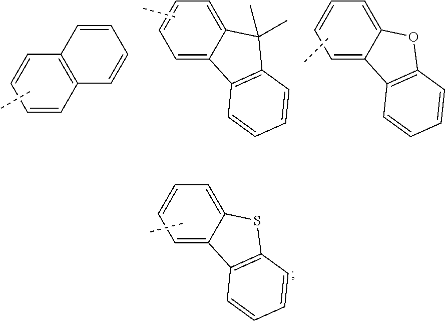

wherein, the dotted line represents a connecting bond of the groups.

Preferably, in compound I, the R¹, R² and R³ are independently selected from the group consisting of one of methyl, ethyl or phenyl, preferably are all methyl.

Preferably, in compound I, the L¹ is selected from the group consisting of one of the following substituted or unsubstituted groups: phenylene, biphenylene, naphthylene, dibenzofuranylene, dibenzothiophenylene, and 9,9dimethylfluorenylene.

Preferably, in compound I, the L² is selected from the group consisting of one of a single bond, a substituted or unsubstituted C6-C20 arylene or a substituted or unsubstituted C3-C20 heteroarylene, preferably a single bond or phenylene.

Preferably, the compound II is a compound formed by substituting at least one Rx at any substitutable position in the structure as shown in Formula (3-1);

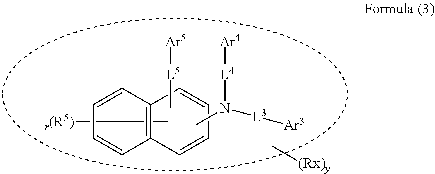

Preferably, the Rx is selected from the group consisting of any one of the following groups:

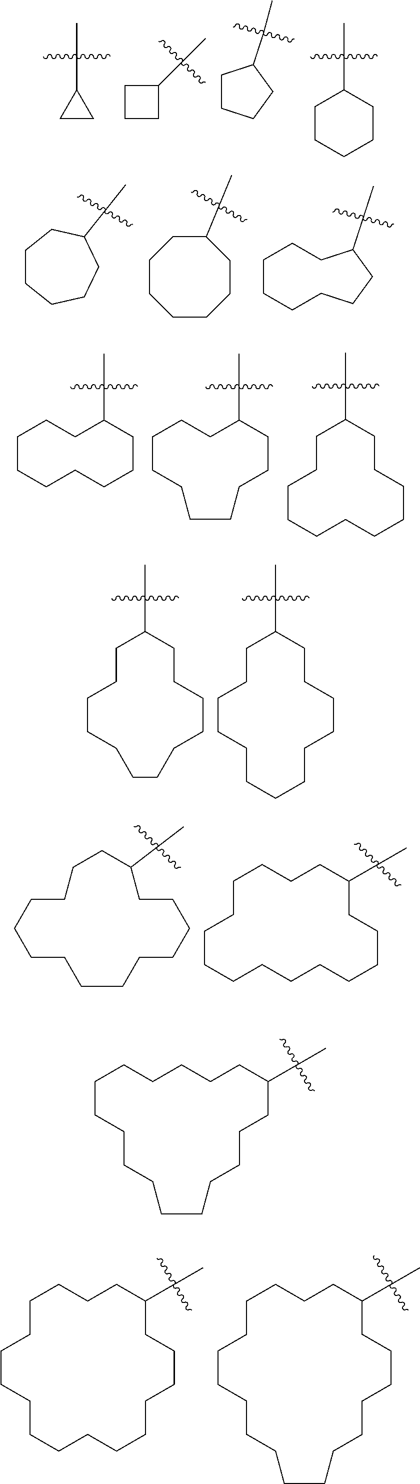

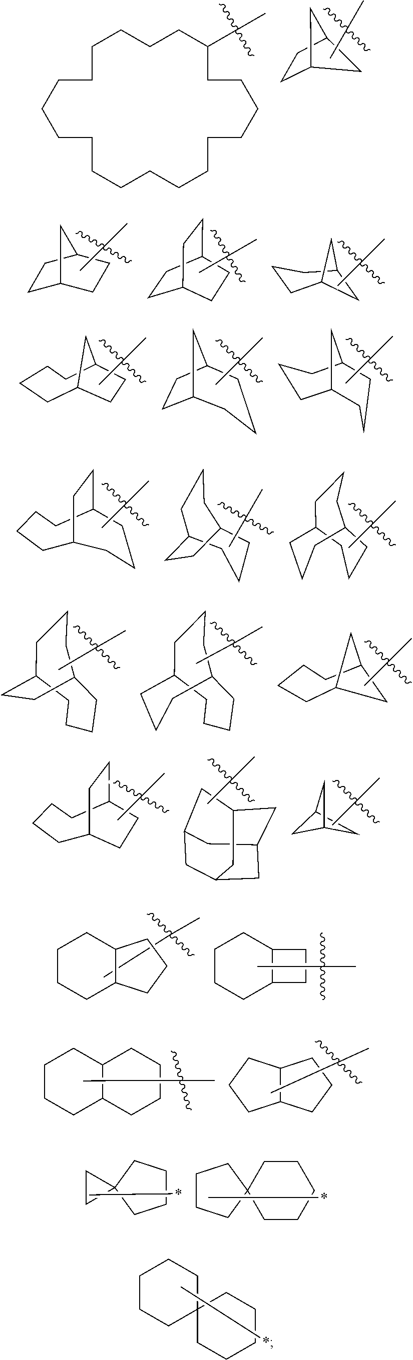

wherein, the wavy line or * label represents a connecting bond of the groups.

Preferably, the Ar³ to Ar⁵ are each independently selected from the group consisting of any of the following substituted or unsubstituted groups:

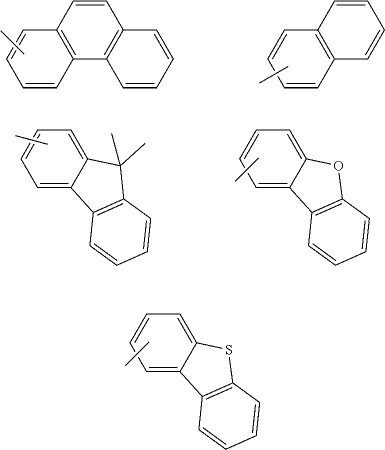

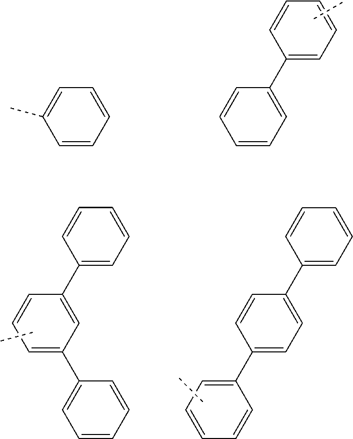

wherein, the dotted line represents a connecting bond of the groups.

Preferably, the L³ and L⁴ are each independently selected from the group consisting of a single bond, phenylene or naphthylene, preferably is a single bond.

Preferably, the L⁵ is a single bond.

The OLED includes an organic material layer at the first electrode and the second electrode as well as between the electrodes. The organic material layer can be divided into multiple regions. For example, the organic material layer may include a hole transporting region, a light emitting layer, and an electron transporting region.

In specific embodiments, a substrate can be used below the first electrode or above the second electrode. The substrate is glass or a polymer material with excellent mechanical strength, thermal stability, water resistance and transparency. In addition, the substrate used as the display can also be equipped with thin film transistors (TFTs).

The first electrode can be formed by sputtering or depositing a material used as the first electrode on the substrate. When the first electrode is used as the anode, an oxide transparent conductive material such as indium tin oxide (ITO), indium zinc oxide (IZO), tin dioxide (SnO₂), zinc oxide (ZnO), etc., and any combination thereof can be used. When the first electrode is used as the cathode, a metal or alloy such as magnesium (Mg), silver (Ag), aluminum (Al), aluminum-lithium (Al—Li), calcium (Ca), ytterbium (Yb) magnesium-indium (Mg—In), magnesium-silver (Mg—Ag), etc., as well as any combination thereof can be used.

The organic material layer can be formed on the electrode by methods such as vacuum thermal evaporation, rotary coating, and printing, etc. The compounds used as the organic material layer can be an organic small molecule, an organic macromolecule and a polymer, and a combination thereof.

The hole transporting region is located between the anode and the light emitting layer. The hole transporting region can be a hole transporting layer (HTL) with a single-layer structure, including a single-layer hole transporting layer containing only one compound and a single-layer hole transporting layer containing multiple compounds. The hole transporting region can be a multilayer structure including at least one of a hole injection layer (HIL), a hole transporting layer (HTL) and an electron blocking layer (EBL), wherein HIL is located between anode and HTL, and EBL is located between HTL and light emitting layer.

The hole transporting layer material can be selected from the group consisting of, but not limited to a phthalocyanine derivative such as CuPc, a conductive polymer or a polymer containing a conductive dopant such as polyphenylenevinylene, polyaniline/dodecyl benzene sulfonic acid (Pani/DBSA), poly(3,4-ethylenedioxythiophene)/poly(4-styrenesulfonate) (PEDOT/PSS), polyaniline/camphor sulfonic acid (Pani/CSA), polyaniline/poly(4-styrenesulfonate) (Pani/PSS), an aromatic amine derivative, wherein the aromatic amine derivative is compounds as shown in the above mentioned HT-1 to HT-51; or any combination thereof.

The hole injection layer is located between the anode and the hole transporting layer. The hole injection layer can be a single compound material or a combination of multiple compounds. For example, the hole injection layer can adopt one or more compounds of the above mentioned HT-1 to HT-51, or one or more compounds of the above mentioned HI-1 to HI-3; or one or more compounds of HT-1 to HT-51 doped with one or more compounds of the above mentioned HI-1 to HI-3.

The light emitting layer includes a light emitting dye (i.e. a dopant) that can emit spectra of different wavelengths, and can also include a host material (Host). The light emitting layer may be a monochromatic light emitting layer emitting a single color such as red, green, and blue, etc. The monochromatic light emitting layers with different colors can be arranged in a plane according to the pixel graphics, or stacked together to forma color light emitting layer. When the light emitting layers of different colors are stacked together, they can be separated from each other or connected to each other. The light emitting layer can also be a single color light emitting layer that can emit different colors such as red, green, and blue, etc., at the same time.

According to different technologies, the light emitting layer material can be a phosphorescent electroluminescent material. In an OLED device, a single light emitting technology or a combination of different light emitting technologies can be used. These different light emitting materials classified by technologies can emit light of the same color or light of different colors.

In one aspect of the disclosure, the second host material of the light emitting layer is a phosphorescent material, and the phosphorescent material is selected from the group consisting of, but not limited to one or a combination of more of PH-1 to PH-86.

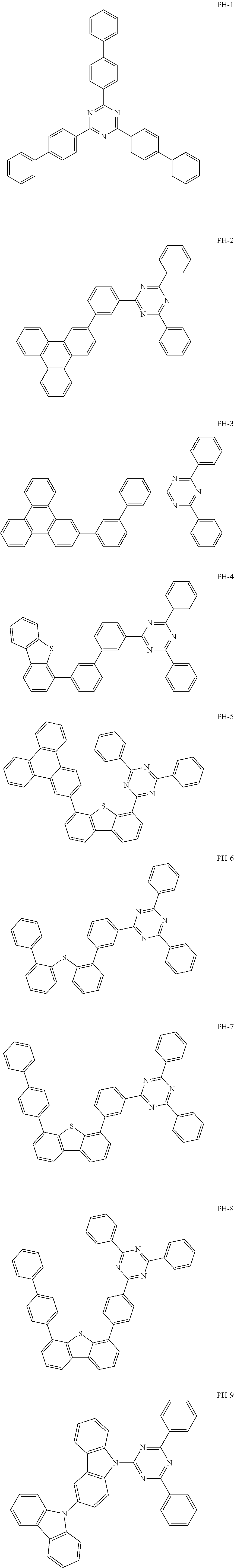

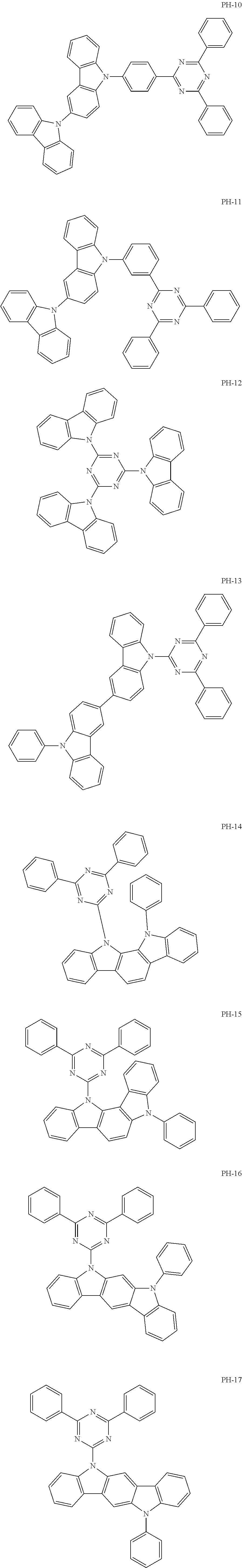

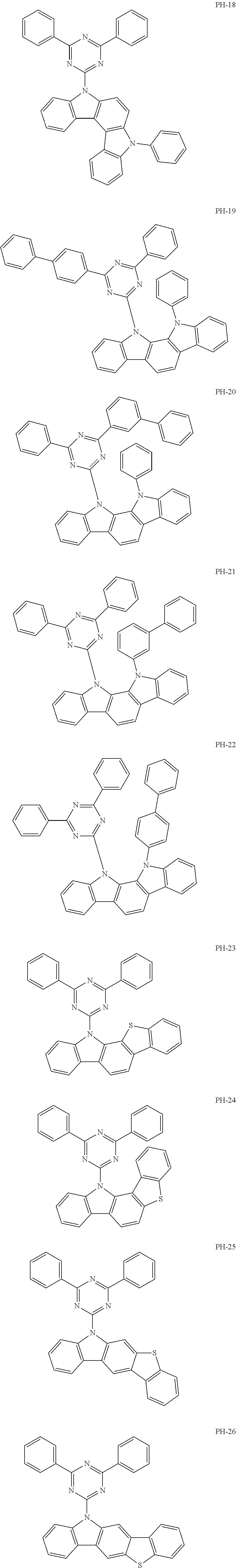

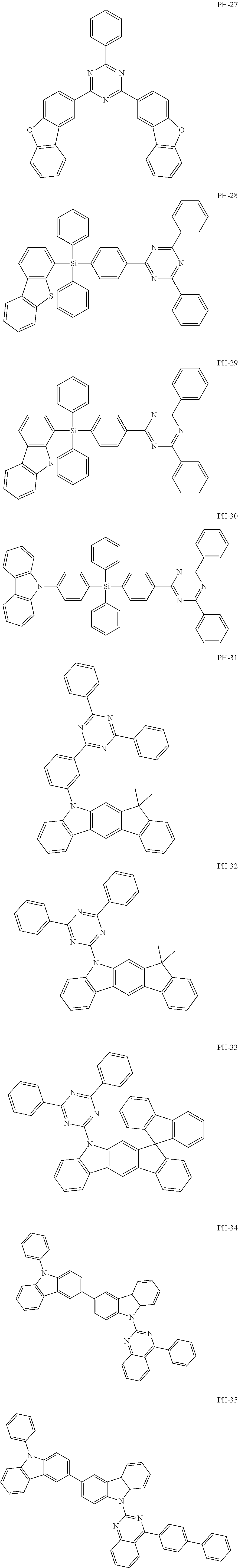

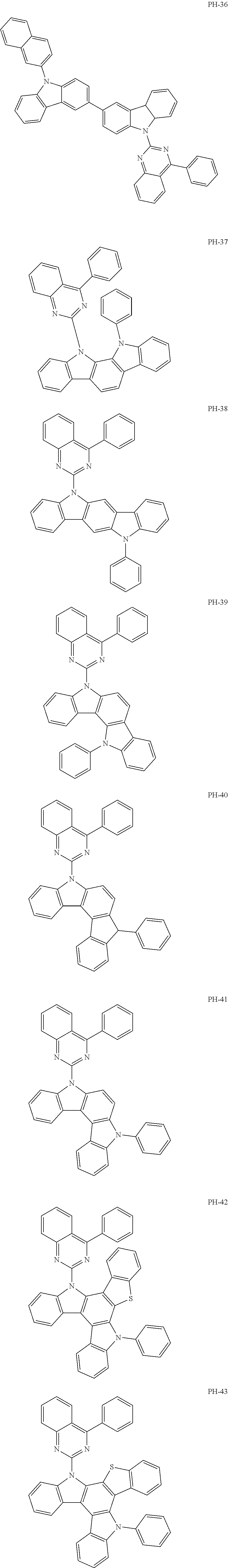

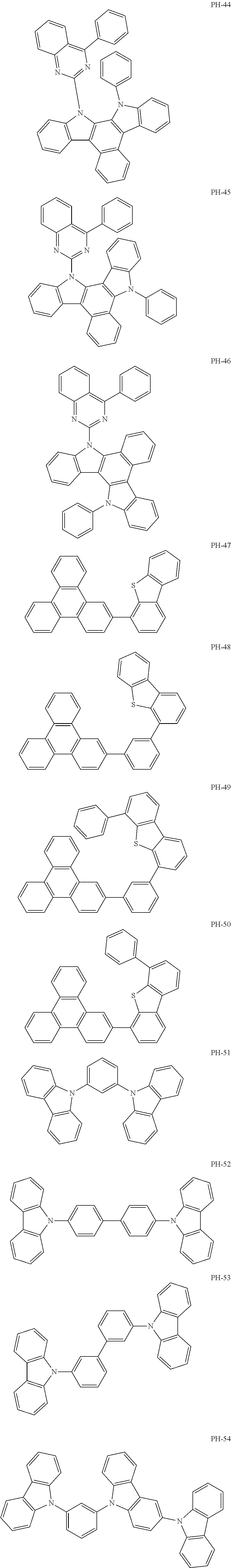

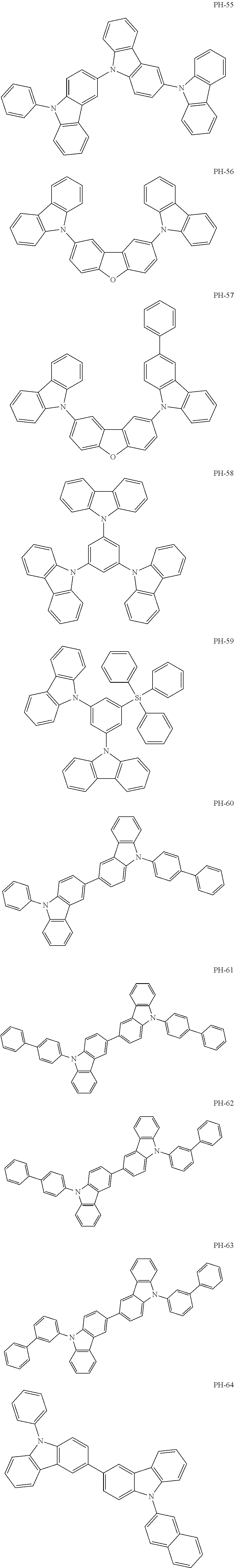

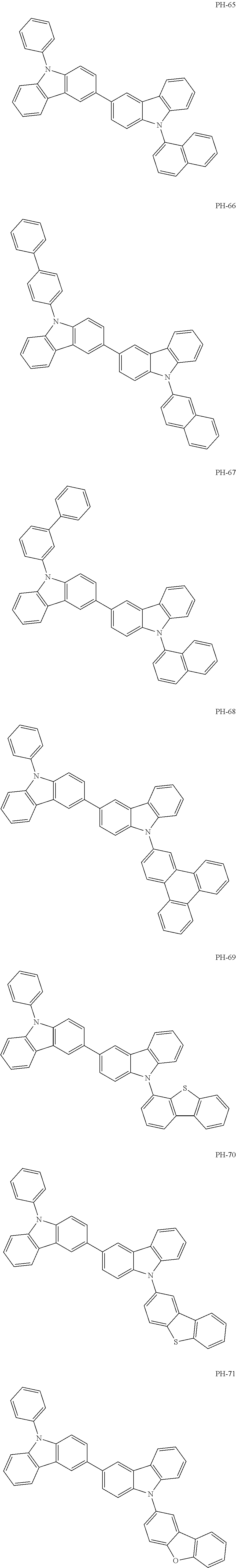

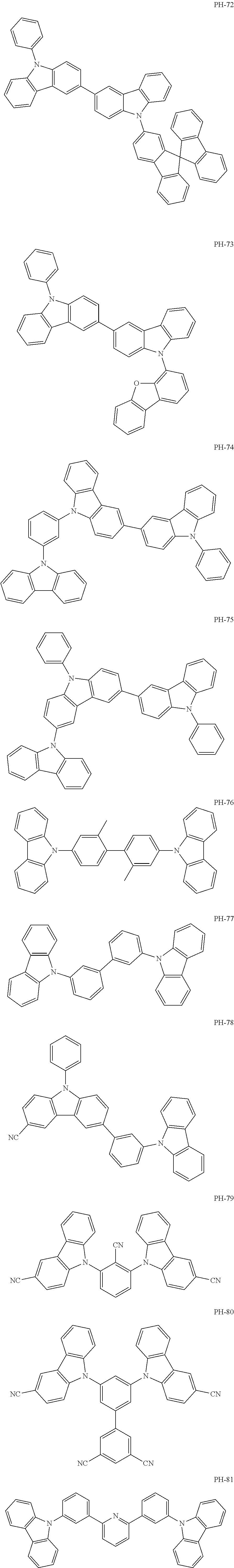

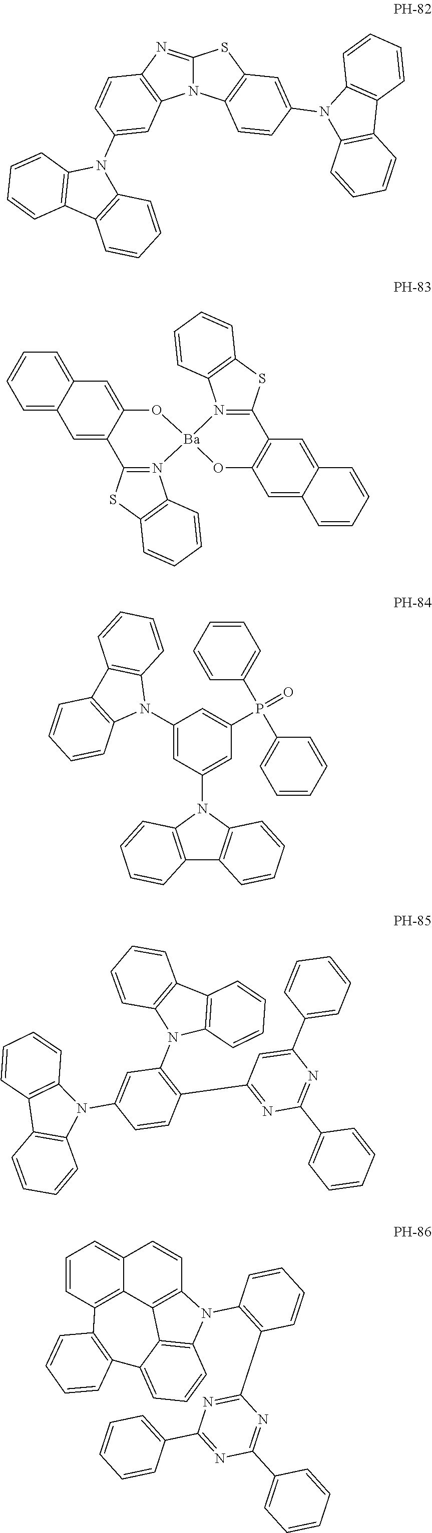

In one aspect of the disclosure, the phosphorescent electroluminescence technology is adopted for the light emitting layer. The phosphorescent dopant of the light emitting layer thereof can be selected from the group consisting of, but not limited to one or a combination of more of the above mentioned GPD-1 to GPD-47.

In one aspect of the disclosure, the phosphorescent electroluminescence technology is adopted for the light emitting layer. The phosphorescent dopant of the light emitting layer thereof can be selected from the group consisting of, but not limited to one or a combination of more of the above mentioned RPD-1 to RPD-28.

In one aspect of the disclosure, the phosphorescent electroluminescence technology is adopted for the light emitting layer. The phosphorescent dopant of the light emitting layer thereof can be selected from the group consisting of, but not limited to one or a combination of more of the above mentioned YPD-1 to YPD-11.

In one aspect of the disclosure, the thermally activated delayed fluorescent light emitting technology is adopted for the light emitting layer. The host material of the light emitting layer thereof is selected from the group consisting of, but not limited to one or a combination of more of the above mentioned PH-1 to PH-86.

In one aspect of the disclosure, the electron blocking layer (EBL) is located between the hole transporting layer and the light emitting layer. The electron blocking layer can adopt, but is not limited to one or more compounds contained in the above mentioned compound I and compound IL.

The OLED organic material layer may also include an electron transporting region between the light emitting layer and the cathode. The electron transporting region can be an electron transporting layer (ETL) with a single-layer structure, including a single-layer electron transporting layer containing only one compound and a single-layer electron transporting layer containing multiple compounds. The electron transporting region may also be a multilayer structure comprising at least one of an electron injection layer (EIL), and an electron transporting layer (ETL).

In one aspect of the disclosure, the electron transporting layer material can be selected from the group consisting of, but is not limited to one or a combination of more of the above mentioned ET-1 to ET-65.

The device can also include an electron injection layer between the electron transporting layer and the cathode. The electron injection layer material includes, but is not limited to, one or a combination of more of: LiQ, LiF, NaCl, CsF, Li₂O, Cs₂CO₃, BaO, Na, Li, Ca, Mg, Yb as listed below.

In another embodiment of the disclosure, a display device is provided. The display device includes the organic electroluminescent device as described in the first object of the disclosure.

Compared with the prior art, the above preferred embodiments of the disclosure have the following beneficial effects:

in the disclosure, compound I and compound II are used as the materials of the organic layer at the same time, wherein compound I can adjust the distribution of carriers inside the light emitting layer, so as to regulate the carrier composite region, and it also has a higher spatial packing structure. At the same time, compound II has a higher molecular plane unfolding properties, so as to achieve faster transferring of holes, improve hole mobility, and has a higher triplet energy level, which can block excess excitons. The combination of compound I and compound II can effectively improve the efficiency of the devices.

### BRIEF DESCRIPTION OF THE DRAWINGS

The accompanying drawings of the description, which form a part of the present application, are used to provide a further understanding of the disclosure. The illustrative examples and their descriptions of the disclosure are used to explain the disclosure, and do not constitute an improper limitation to the disclosure. In the drawings:

FIG. 1 is a structural representation of the organic electroluminescent device provided in a specific embodiment of the disclosure;

wherein, 1—a glass substrate with an anode; 2—a hole injection layer; 3—a hole transporting layer; 4—an electron blocking layer; 5—a light emitting layer; 6—an electron transporting layer; 7—an electron injection layer; 8—a cathode layer; 9—an external power supply.

### DETAILED DESCRIPTION OF THE EMBODIMENTS

It should be noted that the embodiments and features in the embodiments in the application can be combined with each other without conflict. The disclosure will be described in detail below with reference to the embodiments.

The representative synthesis route of compound of Formula I according to the disclosure is as follows:

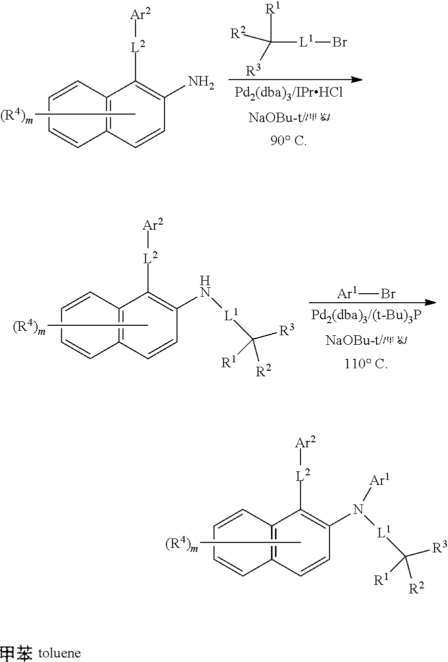

wherein, R₁, R², R₃, R₄, L¹, L², Ar¹ and Ar² have the same meaning as the symbols in Formula I; Pd₂(dba)₃ represents tris(dibenzyl acetone) dipalladium(0), IPr·HCl represents 1, bis(2,-diisopropylphenyl)imidazolium chloride, NaOBu-t represents sodium tert-butoxide, and (t-Bu)₃P represents tri(tert-butyl)phosphine.

More specifically, the disclosure provides a specific synthesis method of the representative compounds in the following synthesis examples. Solvents and reagents used in the following synthesis examples, such as for example, 3-bromo-9,9-dimethylfluorene, 1, bis (2, diisopropyl phenyl) imidazolium chloride, tris(dibenzyl acetone) dipalladium(O), toluene, methanol, ethanol, tri(tert-butyl)phosphine, potassium/sodium tert-butoxide, etc., can be purchased or customized from the domestic chemical product market, for example, purchased from Sinopharm Reagent Co., Ltd., SigmaAldrich Co., Ltd., and J&K Scientific Co., Ltd., and Intermediates M1 to M7 were customized through reagent companies. In addition, they can also be synthesized by those skilled in the art through well-known methods.

### Synthesis Example 1: Synthesis of Compound P1

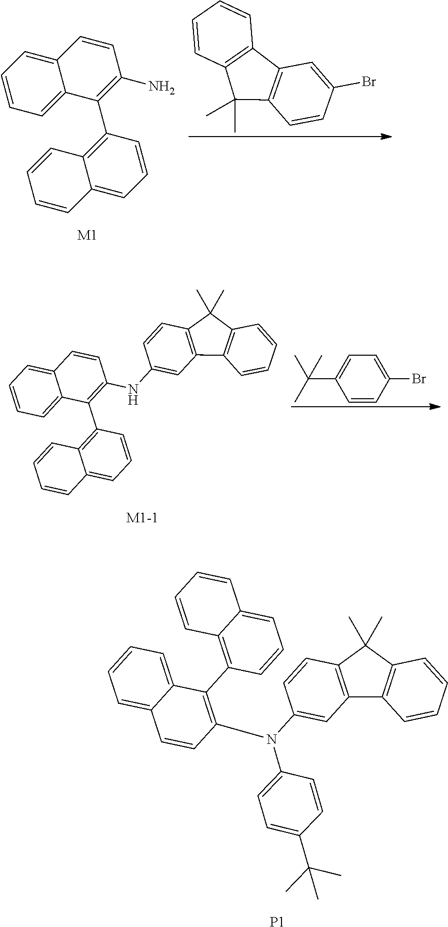

Into a 1000 mL single-necked bottle, 13.5 g (50 mmol) of M1, 13.6 g (50 mmol) of 3-bromo-9,9-dimethylfluorene, 0.9 g (1 mmol) of tris(dibenzylideneacetone)dipalladium (i.e. Pd₂(dba)₃), 0.5 g of IPr·HCl, 500 mL of toluene, and 14.4 g (150 mmol) of sodium tert-butoxide (NaOBu-t) were added, vacuumized and replaced with nitrogen for three times, and the reaction was heated up to 90° C. for reacting 5 hours. After the reaction was completed, it was stopped. The reaction was cooled to room temperature, the reaction solution was layered, the organic phase was concentrated and methanol was added and stirred for 1 hour, suction filtered to obtain M1-1 as a light yellow powder.

Into a 1000 mL single-necked bottle, 23 g (50 mmol) of M1-1, 11 g (50 mmol) of 4-bromo-4′-tert-butylbiphenyl, 0.9 g (1 mmol) of tris(dibenzylideneacetone)dipalladium (i.e. Pd₂(dba)₃), 0.5 mL of Tri-tert-butylphosphine ((t-Bu)₃P), 500 mL of toluene, and 14.4 g (150 mmol) of sodium tert-butoxide (NaOBu-t) were added, vacuumized and replaced with nitrogen for three times, and the reaction was heated up to 110° C. for reacting 5 hours. After the reaction was completed, it was stopped. The reaction was cooled to room temperature, the reaction solution was layered, the organic phase was concentrated and methanol was added and stirred for 1 hour, suction filtered to obtain P1 as a light yellow powder.

M/Z theoretical value: 593; ZAB-HS Model Mass Spectrometer (Manufactured by Micromass, British) M/Z found: 594.

### Synthesis Example 2: Synthesis of Compound P2

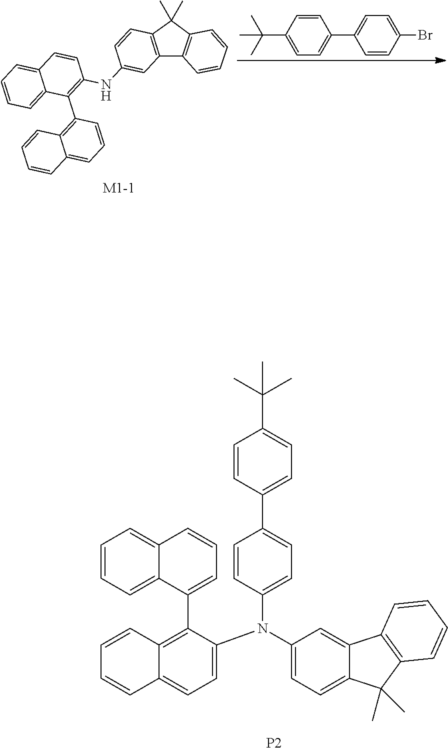

Into a 1000 mL single-necked bottle, 23 g (50 mmol) of M1-1, 14.4 g (50 mmol) of 4-bromo-4′-tert-butylbiphenyl, 0.9 g (1 mmol) of tris(dibenzylideneacetone)dipalladium (i.e. Pd2(dba)3), 0.5 mL of Ti-tert-butylphosphine((t-Bu)3P), 500 mL of toluene, and 14.4 g (150 mmol) of sodium tert-butoxide(NaOBu-t) were added, vacuumized and replaced with nitrogen for three times, and the reaction was heated up to 110° C. for reacting 5 hours. After the reaction was completed, it was stopped. The reaction was cooled to room temperature, the reaction solution was layered, the organic phase was concentrated and methanol was added and stirred for 1 hour, suction filtered to obtain P2 as a light yellow powder.

M/Z theoretical value: 669; ZAB-HS Model Mass Spectrometer (Manufactured by Micromass, British) M/Z found: 670.

### Synthesis Example 3: Synthesis of Compound P5

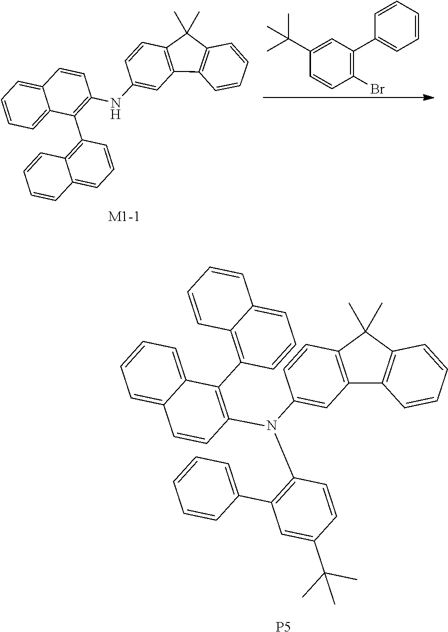

Into a 1000 mL single-necked bottle, 23 g (50 mmol) of M1-1, 14.5 g (50 mmol) of 2-bromo-5-tert-butylbiphenyl, 0.9 g (1 mmol) of tris(dibenzylideneacetone)dipalladium (i.e. Pd₂(dba)₃), 0.5 mL of Tri-tert-butylphosphine((t-Bu)₃P), 500 mL of toluene, 14.4 g (150 mmol) of sodium tert-butoxide(NaOBu-t) were added, vacuumized and replaced with nitrogen for three times, and the reaction was heated up to 110° C. for reacting 5 hours. After the reaction was completed, it was stopped. The reaction was cooled to room temperature, the reaction solution was layered, the organic phase was concentrated and methanol was added and stirred for 1 hour, suction filtered to obtain P5 as a light yellow powder.

M/Z theoretical value: 669; ZAB-HS Model Mass Spectrometer (Manufactured by Micromass, British) M/Z found: 670.

### Synthesis Example 4: Synthesis of Compound P6

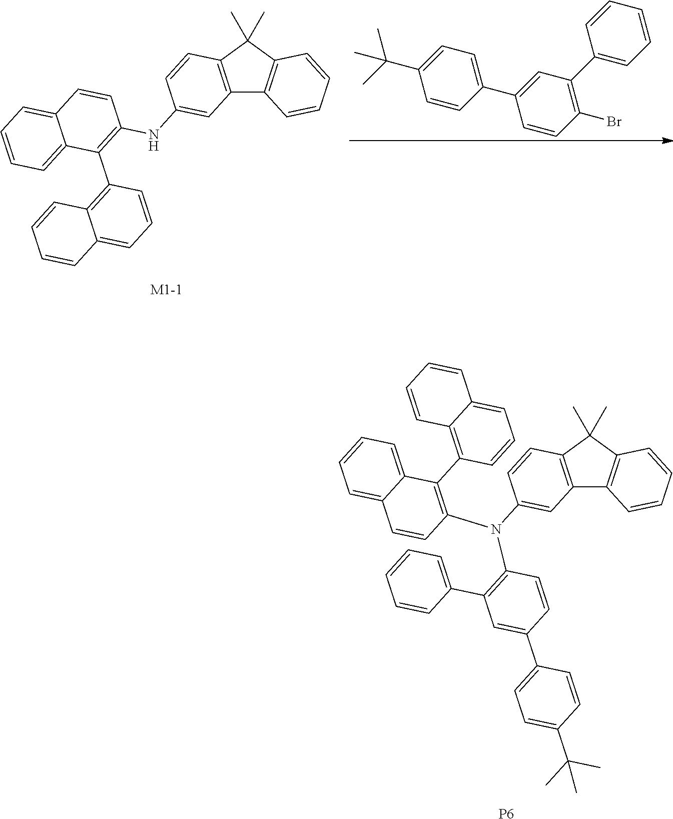

Into a 1000 mL single-necked bottle, 23 g (50 mmol) of M1-1, 18.5 g (50 mmol) of 4-bromo-3phenyl-4′-tert-butylbiphenyl, 0.9 g (1 mmol) of tris(dibenzylideneacetone)dipalladium (i.e. Pd₂(dba)₃), 0.5 mL of Tri-tert-butylphosphine((t-Bu)₃P), 500 mL of toluene, and 14.4 g (150 mmol) of sodium tert-butoxide(NaOBu-t) were added, vacuumized and replaced with nitrogen for three times, and the reaction was heated up to 110° C. for reacting 5 hours. After the reaction was completed, it was stopped. The reaction was cooled to room temperature, the reaction solution was layered, the organic phase was concentrated and methanol was added and stirred for 1 hour, suction filtered to obtain P6 as a light yellow powder.

M/Z theoretical value: 745; ZAB-HS Model Mass Spectrometer (Manufactured by Micromass, British) M/Z found: 746.

### Synthesis Example 5: Synthesis of Compound P7

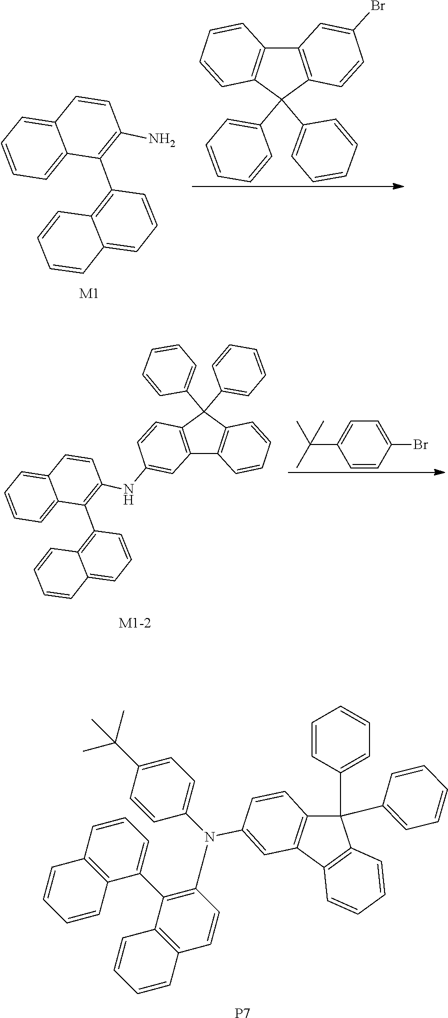

Into a 1000 mL single-necked bottle, 13.5 g (50 mmol) of M1, 20 g (50 mmol) of 3-bromo-9,9-diphenylfluorene, 0.9 g (1 mmol) of tris(dibenzylideneacetone)dipalladium (i.e. Pd₂(dba)₃), 0.5 g of IPr·HCl, 500 mL of toluene, and 14.4 g (150 mmol) of sodium tert-butoxide(NaOBu-t) were added, vacuumized and replaced with nitrogen for three times, and the reaction was heated up to 90° C. for reacting 5 hours. After the reaction was completed, it was stopped. The reaction was cooled to room temperature, the reaction solution was layered, the organic phase was concentrated and methanol was added and stirred for 1 hour, suction filtered to obtain M1-2 as a light yellow powder.

Into a 1000 mL single-necked bottle, 29 g (50 mmol) of M1-2, 11 g (50 mmol) of 4-bromo-4′-tert-butylbiphenyl, 0.9 g (1 mmol) of tris(dibenzylideneacetone)dipalladium (i.e. Pd₂(dba)₃), 0.5 mL of Tri-tert-butylphosphine ((t-Bu)₃P), 500 mL of toluene, and 14.4 g (150 mmol) of sodium tert-butoxide (NaOBu-t) were added, vacuumized and replaced with nitrogen for three times, and the reaction was heated up to 110° C. for reacting 5 hours. After the reaction was completed, it was stopped. The reaction was cooled to room temperature, the reaction solution was layered, the organic phase was concentrated and methanol was added and stirred for 1 hour, suction filtered to obtain P7 as a light yellow powder.

M/Z theoretical value: 717; ZAB-HS Model Mass Spectrometer (Manufactured by Micromass, British) M/Z found: 718.

### Synthesis Example 6: Synthesis of Compound P15

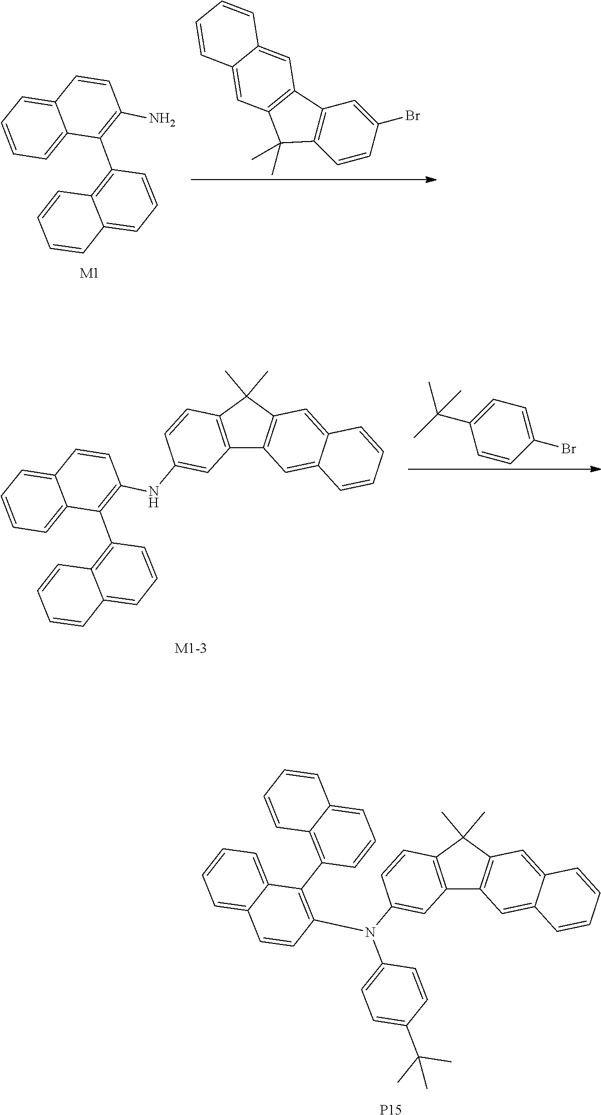

Into a 1000 mL single-necked bottle, 13.5 g (50 mmol) of M1, 16.5 g (50 mmol) of 3-bromo-[6,7]-benzo-9,9-dimethylfluorene, 0.9 g (1 mmol) of tris(dibenzylideneacetone)dipalladium (i.e. Pd₂(dba)₃), 0.5 g of IPr·HCl, 500 mL of toluene, and 14.4 g (150 mmol) of sodium tert-butoxide(NaOBu-t) were added, vacuumized and replaced with nitrogen for three times, and the reaction was heated up to 90° C. for reacting 5 hours. After the reaction was completed, it was stopped. The reaction was cooled to room temperature, the reaction solution was layered, the organic phase was concentrated and methanol was added and stirred for 1 hour, suction filtered to obtain M1-3 as a light yellow powder.

Into a 1000 mL single-necked bottle, 25.5 g (50 mmol) of M1-3, 11 g (50 mmol) of 4-bromo-tert-butylbenzene, 0.9 g (1 mmol) of tris(dibenzylideneacetone)dipalladium (i.e. Pd₂(dba)₃), 0.5 mL of Tri-tert-butylphosphine((t-Bu)₃P), 500 mL of toluene, and 14.4 g (150 mmol) of sodium tert-butoxide(NaOBu-t) were added, vacuumized and replaced with nitrogen for three times, and the reaction was heated up to 110° C. for reacting 5 hours. After the reaction was completed, it was stopped. The reaction was cooled to room temperature, the reaction solution was layered, the organic phase was concentrated and methanol was added and stirred for 1 hour, suction filtered to obtain P15 as a light yellow powder.

M/Z theoretical value: 643; ZAB-HS Model Mass Spectrometer (Manufactured by Micromass, British) M/Z found: 644.

### Synthesis Example 7: Synthesis of Compound P37

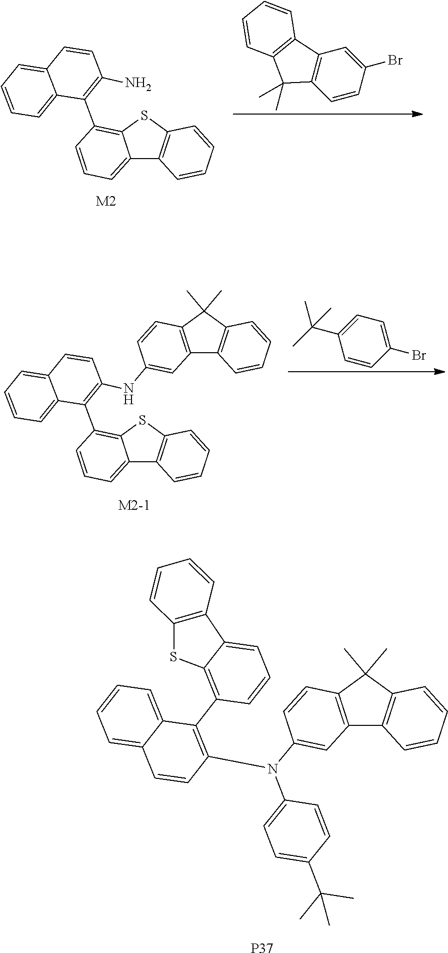

Into a 1000 mL single-necked bottle, 16 g (50 mmol) of M2, 13.5 g (50 mmol) of 3-bromo-9,9-dimethylfluorene, 0.9 g (1 mmol) of tris(dibenzylideneacetone)dipalladium (i.e. Pd₂(dba)₃), 0.5 g of IPr·HCl, 500 mL of toluene, and 14.4 g (150 mmol) of sodium tert-butoxide (NaOBu-t) were added, vacuumized and replaced with nitrogen for three times, and the reaction was heated up to 90° C. for reacting 5 hours. After the reaction was completed, it was stopped. The reaction was cooled to room temperature, the reaction solution was layered, the organic phase was concentrated and methanol was added and stirred for 1 hour, suction filtered to obtain M2-1 as a light yellow powder.

Into a 1000 mL single-necked bottle, 26 g (50 mmol) of M2-1, 11 g (50 mmol) of 4-bromo-tert-butylbenzene, 0.9 g (1 mmol) of tris(dibenzylideneacetone)dipalladium (i.e. Pd₂(dba)₃), 0.5 mL of Tri-tert-butylphosphine((t-Bu)₃P), 500 mL of toluene, and 14.4 g (150 mmol) of sodium tert-butoxide(NaOBu-t) were added, vacuumized and replaced with nitrogen for three times, and the reaction was heated up to 110° C. for reacting 5 hours. After the reaction was completed, it was stopped. The reaction was cooled to room temperature, the reaction solution was layered, the organic phase was concentrated and methanol was added and stirred for 1 hour, suction filtered to obtain P37 as a light yellow powder.

M/Z theoretical value: 649; ZAB-HS Model Mass Spectrometer (Manufactured by Micromass, British) M/Z found: 650.

### Synthesis Example 8: Synthesis of Compound P38

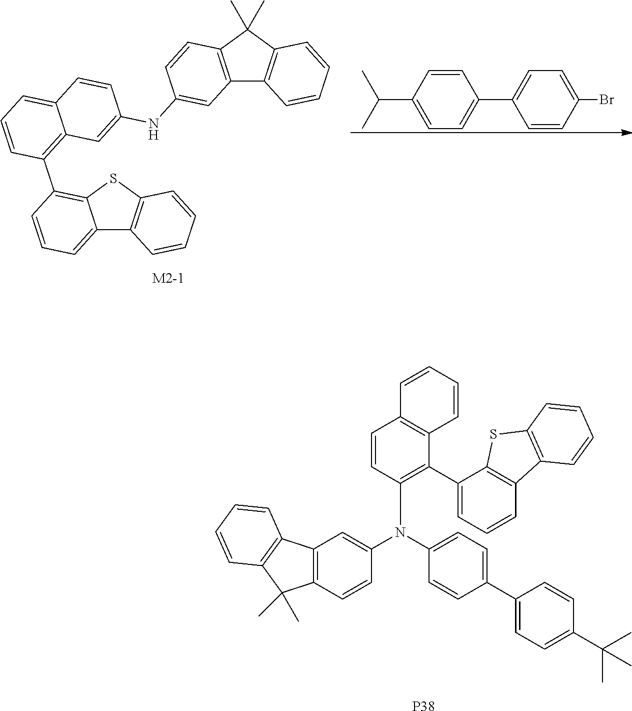

Into a 1000 mL single-necked bottle, 26 g (50 mmol) of M2-1, 14.5 g (50 mmol) of 4-bromo-4′-tert-butylbiphenyl, 0.9 g (1 mmol) of tris(dibenzylideneacetone)dipalladium (i.e. Pd₂(dba)₃), 0.5 mL of Tri-tert-butylphosphine((t-Bu)₃P), 500 mL of toluene, and 14.4 g (150 mmol) of sodium tert-butoxide(NaOBu-t) were added, vacuumized and replaced with nitrogen for three times, and the reaction was heated up to 110° C. for reacting 5 hours. After the reaction was completed, it was stopped. The reaction was cooled to room temperature, the reaction solution was layered, the organic phase was concentrated and methanol was added and stirred for 1 hour, suction filtered to obtain P38 as a light yellow powder.

M/Z theoretical value: 725; ZAB-HS Model Mass Spectrometer (Manufactured by Micromass, British) M/Z found: 726.

### Synthesis Example 9: Synthesis of Compound P74

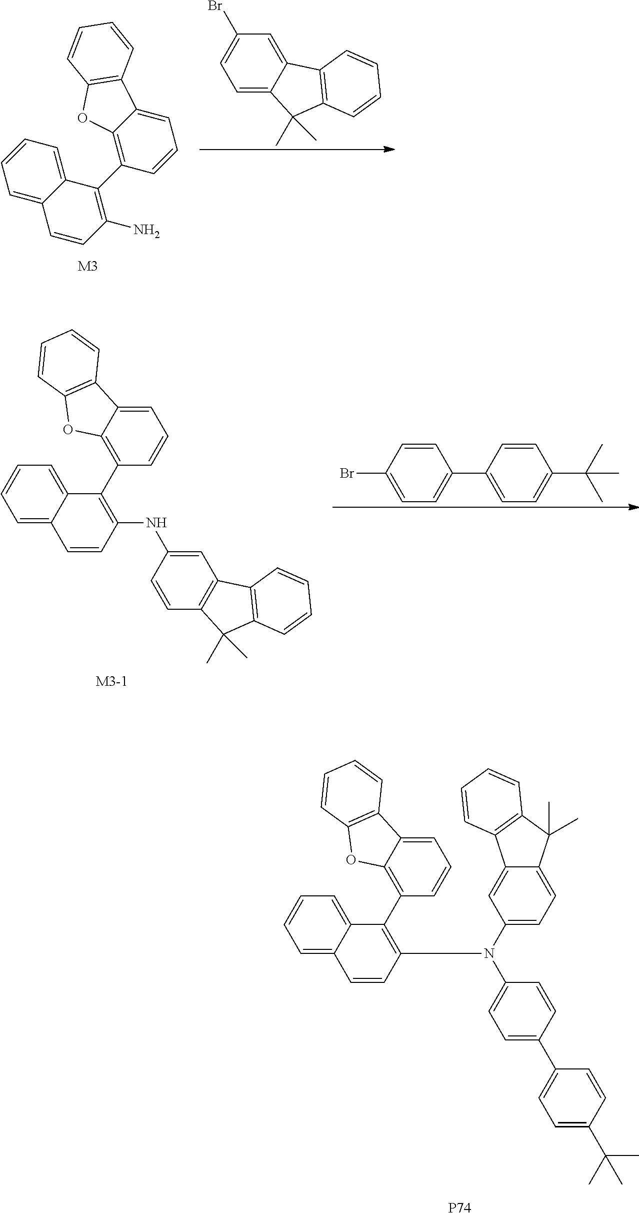

Into a 1000 mL single-necked bottle, 15.5 g (50 mmol) of M3, 13.5 g (50 mmol) of 3-bromo-9,9-dimethylfluorene, 0.9 g (1 mmol) of tris(dibenzylideneacetone)dipalladium (i.e. Pd₂(dba)₃), 0.5 g of IPr·HCl, 500 mL of toluene, and 14.4 g (150 mmol) of sodium tert-butoxide (NaOBu-t) were added, vacuumized and replaced with nitrogen for three times, and the reaction was heated up to 90° C. for reacting 5 hours. After the reaction was completed, it was stopped. The reaction was cooled to room temperature, the reaction solution was layered, the organic phase was concentrated and methanol was added and stirred for 1 hour, suction filtered to obtain M3-1 as a light yellow powder.

Into a 1000 mL single-necked bottle, 25 g (50 mmol) of M3-1, 14.5 g (50 mmol) of 4-bromo-4′-tert-butylbiphenyl, 0.9 g (1 mmol) of tris(dibenzylideneacetone)dipalladium (i.e. Pd₂(dba)₃), 0.5 mL of Tri-tert-butylphosphine((t-Bu)₃P), 500 mL of toluene, and 14.4 g (150 mmol) of sodium tert-butoxide(NaOBu-t) were added, vacuumized and replaced with nitrogen for three times, and the reaction was heated up to 110° C. for reacting 5 hours. After the reaction was completed, it was stopped. The reaction was cooled to room temperature, the reaction solution was layered, the organic phase was concentrated and methanol was added and stirred for 1 hour, suction filtered to obtain P74 as a light yellow powder.

M/Z theoretical value: 709; ZAB-HS Model Mass Spectrometer (Manufactured by Micromass, British) M/Z found: 710.

### Synthesis Example 10: Synthesis of Compound P253

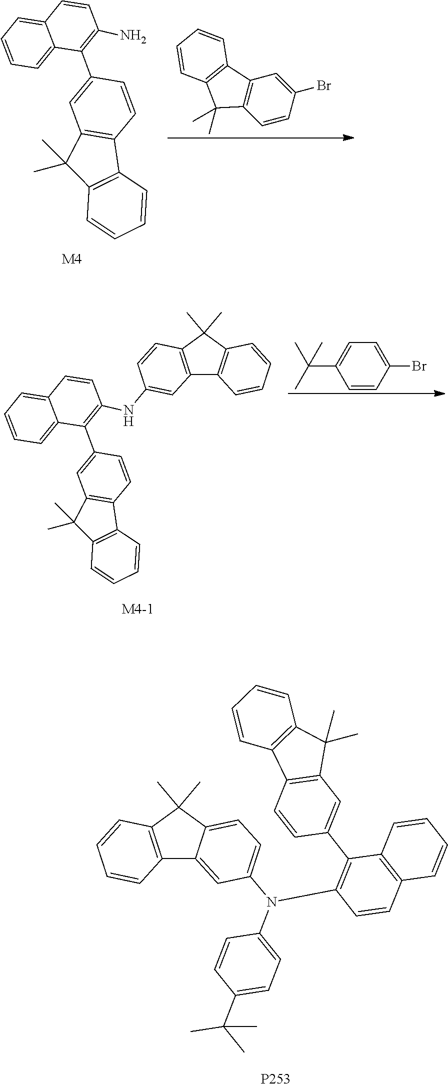

Into a 1000 mL single-necked bottle, 16.5 g (50 mmol) of M4, 13.6 g (50 mmol) of 3-bromo-9,9-dimethylfluorene, 0.9 g (1 mmol) of tris(dibenzylideneacetone)dipalladium (i.e. Pd₂(dba)₃), 0.5 g of IPr·HCl, 500 mL of toluene, and 14.4 g (150 mmol) of sodium tert-butoxide (NaOBu-t) were added, vacuumized and replaced with nitrogen for three times, and the reaction was heated up to 90° C. for reacting 5 hours. After the reaction was completed, it was stopped. The reaction was cooled to room temperature, the reaction solution was layered, the organic phase was concentrated and methanol was added and stirred for 1 hour, suction filtered to obtain M4-1 as a light yellow powder.

Into a 1000 mL single-necked bottle, 26.5 g (50 mmol) of M4-1, 11 g (50 mmol) of 4-bromo-4′-tert-butylbiphenyl, 0.9 g (1 mmol) of tris(dibenzylideneacetone)dipalladium (i.e. Pd₂(dba)₃), 0.5 mL of Tri-tert-butylphosphine ((t-Bu)₃P), 500 mL of toluene, and 14.4 g (150 mmol) of sodium tert-butoxide (NaOBu-t) were added, vacuumized and replaced with nitrogen for three times, and the reaction was heated up to 110° C. for reacting 5 hours. After the reaction was completed, it was stopped. The reaction was cooled to room temperature, the reaction solution was layered, the organic phase was concentrated and methanol was added and stirred for 1 hour, suction filtered to obtain P253 as a light yellow powder.

M/Z theoretical value: 659; ZAB-HS Model Mass Spectrometer (Manufactured by Micromass, British) M/Z found: 660.

### Synthesis Example 11: Synthesis of Compound P284

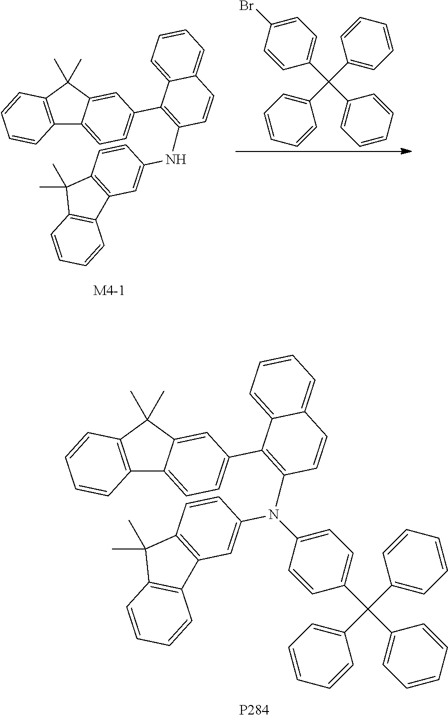 [SMILES: CC1(C)c2ccccc2-c2cc(Nc3ccc4ccccc4c3-c3ccc4c(c3)C(C)(C)c3ccccc3-4)ccc21 ; Brc1ccc(C(c2ccccc2)(c2ccccc2)c2ccccc2)cc1 ; CC1(C)c2ccccc2-c2cc(N(c3ccc(C(c4ccccc4)(c4ccccc4)c4ccccc4)cc3)c3ccc4ccccc4c3-c3ccc4c(c3)C(C)(C)c3ccccc3-4)ccc21 ; "P284"]

Into a 1000 mL single-necked bottle, 26.5 g (50 mmol) of M4-1, 20 g (50 mmol) of 1-(4-bromophenyl)-1,1,1-triphenylmethane, 0.9 g (1 mmol) of tris(dibenzylideneacetone)dipalladium (i.e. Pd₂(dba)₃), 0.5 mL of Tri-tert-butylphosphine((t-Bu)₃P), 500 mL of toluene, and 14.4 g (150 mmol) of sodium tert-butoxide(NaOBu-t) were added, vacuumized and replaced with nitrogen for three times, and the reaction was heated up to 110° C. for reacting 5 hours. After the reaction was completed, it was stopped. The reaction was cooled to room temperature, the reaction solution was layered, the organic phase was concentrated and methanol was added and stirred for 1 hour, suction filtered to obtain P284 as a light yellow powder.

M/Z theoretical value: 845; ZAB-HS Model Mass Spectrometer (Manufactured by Micromass, British) M/Z found: 846.

### Synthesis Example 12: Synthesis of Compound P375

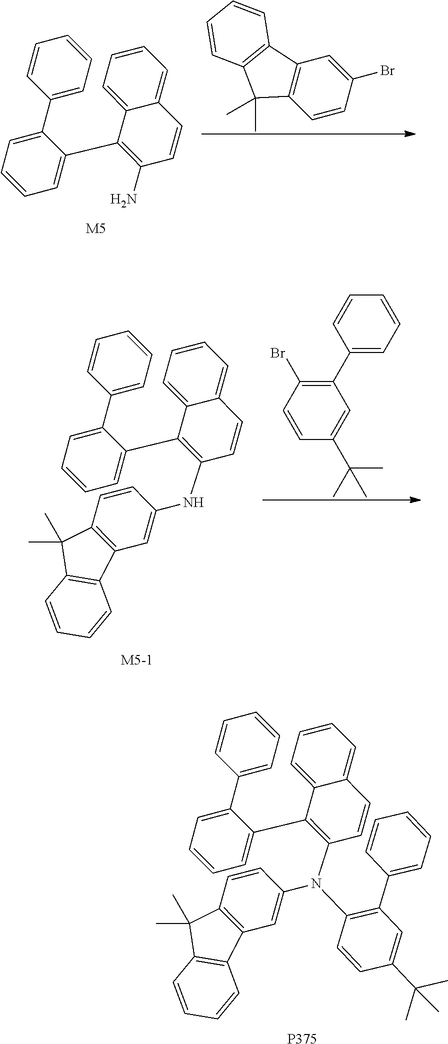

Into a 1000 mL single-necked bottle, 15 g (50 mmol) of M5, 13.5 g (50 mmol) of 3-bromo-9,9-dimethylfluorene, 0.9 g (1 mmol) of tris(dibenzylideneacetone)dipalladium (i.e. Pd₂(dba)₃), 0.5 g of IPr·HCl, 500 mL of toluene, and 14.4 g (150 mmol) of sodium tert-butoxide (NaOBu-t) were added, vacuumized and replaced with nitrogen for three times, and the reaction was heated up to 90° C. for reacting 5 hours. After the reaction was completed, it was stopped. The reaction was cooled to room temperature, the reaction solution was layered, the organic phase was concentrated and methanol was added and stirred for 1 hour, suction filtered to obtain M5-1 as a light yellow powder.

Into a 1000 mL single-necked bottle, 25 g (50 mmol) of MS-1, 14.5 g (50 mmol) of 2-phenyl4-bromotert-butylbenzene, 0.9 g (1 mmol) of tris(dibenzylideneacetone)dipalladium (i.e. Pd₂(dba)₃), 0.5 mL of Tri-tert-butylphosphine((t-Bu)₃P), 500 mL of toluene, and 14.4 g (150 mmol) of sodium tert-butoxide(NaOBu-t) were added, vacuumized and replaced with nitrogen for three times, and the reaction was heated up to 110° C. for reacting 5 hours. After the reaction was completed, it was stopped. The reaction was cooled to room temperature, the reaction solution was layered, the organic phase was concentrated and methanol was added and stirred for 1 hour, suction filtered to obtain P375 as a light yellow powder.

M/Z theoretical value: 695; ZAB-HS Model Mass Spectrometer (Manufactured by Micromass, British) M/Z found: 696.

### Synthesis Example 13: Synthesis of Compound P322

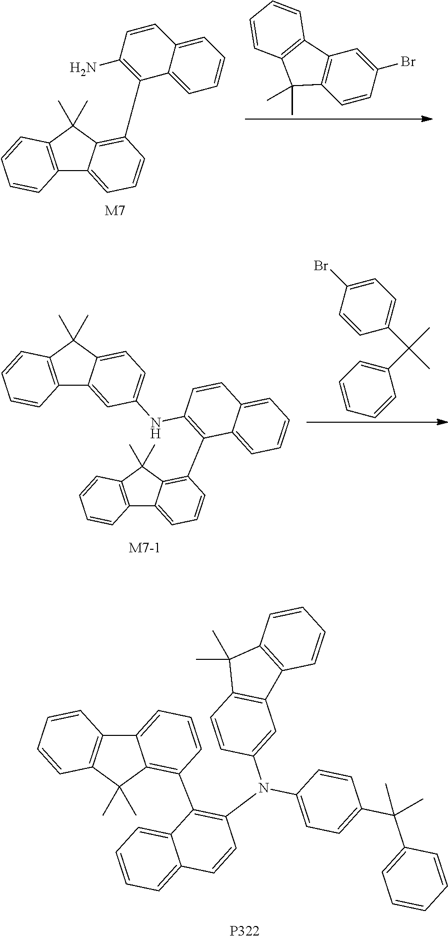

Into a 1000 mL single-necked bottle, 16.5 g (50 mmol) of M7, 13.5 g (50 mmol) of 3-bromo-9,9-dimethylfluorene, 0.9 g (1 mmol) of tris(dibenzylideneacetone)dipalladium (i.e. Pd₂(dba)₃), 0.5 g of IPr·HCl, 500 mL of toluene, and 14.4 g (150 mmol) of sodium tert-butoxide (NaOBu-t) were added, vacuumized and replaced with nitrogen for three times, and the reaction was heated up to 90° C. for reacting 5 hours. After the reaction was completed, it was stopped. The reaction was cooled to room temperature, the reaction solution was layered, the organic phase was concentrated and methanol was added and stirred for 1 hour, suction filtered to obtain M7-1 as a light yellow powder.

Into a 1000 mL single-necked bottle, 26.5 g (50 mmol) of M7-1, 14 g (50 mmol) of 1-(4-bromophenyl)-1,1-dimethyl-1-phenylmethane, 0.9 g (1 mmol) of tris(dibenzylideneacetone)dipalladium (i.e. Pd₂(dba)₃), 0.5 mL of Tri-tert-butylphosphine((t-Bu)₃P), 500 mL of toluene, and 14.4 g (150 mmol) of sodium tert-butoxide(NaOBu-t) were added, vacuumized and replaced with nitrogen for three times, and the reaction was heated up to 110° C. for reacting 5 hours. After the reaction was completed, it was stopped. The reaction was cooled to room temperature, the reaction solution was layered, the organic phase was concentrated and methanol was added and stirred for 1 hour, suction filtered to obtain P322 as a light yellow powder.

M/Z theoretical value: 721; ZAB-HS Model Mass Spectrometer (Manufactured by Micromass, British) M/Z found: 722.

### Synthesis Example 14: Synthesis of Compound P323

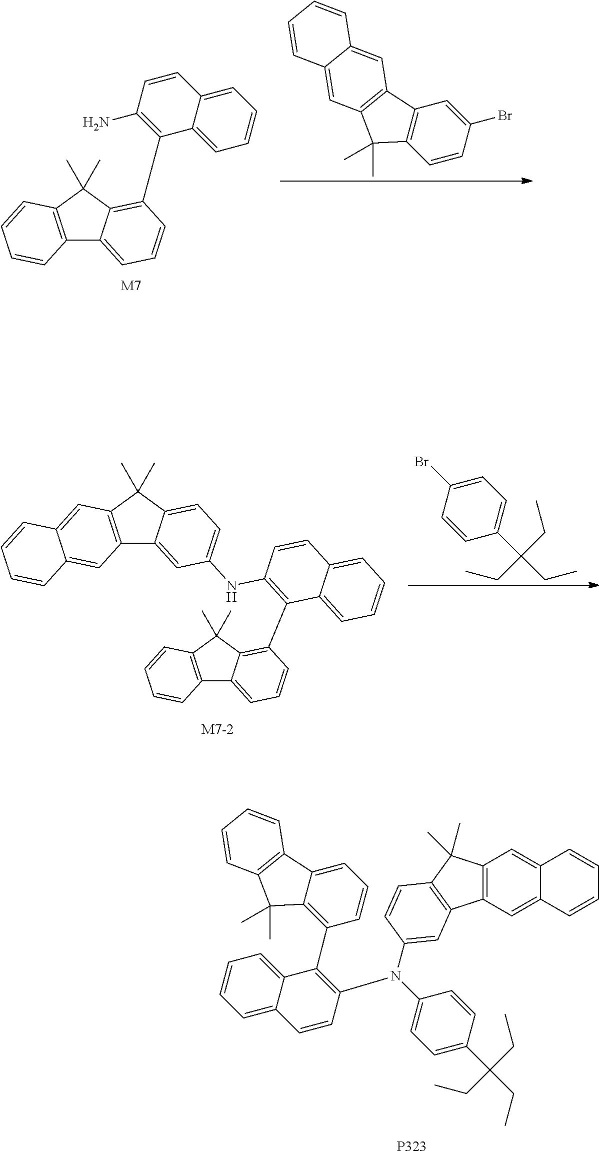

Into a 1000 mL single-necked bottle, 16.5 g (50 mmol) of M7, 16.5 g (50 mmol) of 3-bromo-9,9-dimethyl-6,7-benzofluorene, 0.9 g (1 mmol) of tris(dibenzylideneacetone)dipalladium (i.e. Pd₂(dba)₃), 0.5 g of IPr·HCl, 500 mL of toluene, and 14.4 g (150 mmol) of sodium tert-butoxide(NaOBu-t) were added, vacuumized and replaced with nitrogen for three times, and the reaction was heated up to 90° C. for reacting 5 hours. After the reaction was completed, it was stopped. The reaction was cooled to room temperature, the reaction solution was layered, the organic phase was concentrated and methanol was added and stirred for 1 hour, suction filtered to obtain M7-2 as a light yellow powder.

Into a 1000 mL single-necked bottle, 29 g (50 mmol) of M7-2, 12.7 g (50 mmol) of 1-(4-bromophenyl)-1,1,1-triethylmethane, 0.9 g (1 mmol) of tris(dibenzylideneacetone)dipalladium (i.e. Pd₂(dba)₃), 0.5 mL of Tri-tert-butylphosphine((t-Bu)₃P), 500 mL of toluene, and 14.4 g (150 mmol) of sodium tert-butoxide(NaOBu-t) were added, vacuumized and replaced with nitrogen for three times, and the reaction was heated up to 110° C. for reacting 5 hours. After the reaction was completed, it was stopped. The reaction was cooled to room temperature, the reaction solution was layered, the organic phase was concentrated and methanol was added and stirred for 1 hour, suction filtered to obtain P323 as a light yellow powder.

M/Z theoretical value: 751; ZAB-HS Model Mass Spectrometer (Manufactured by Micromass, British) M/Z found: 752.

### Synthesis Example 15: Synthesis of Compound P20

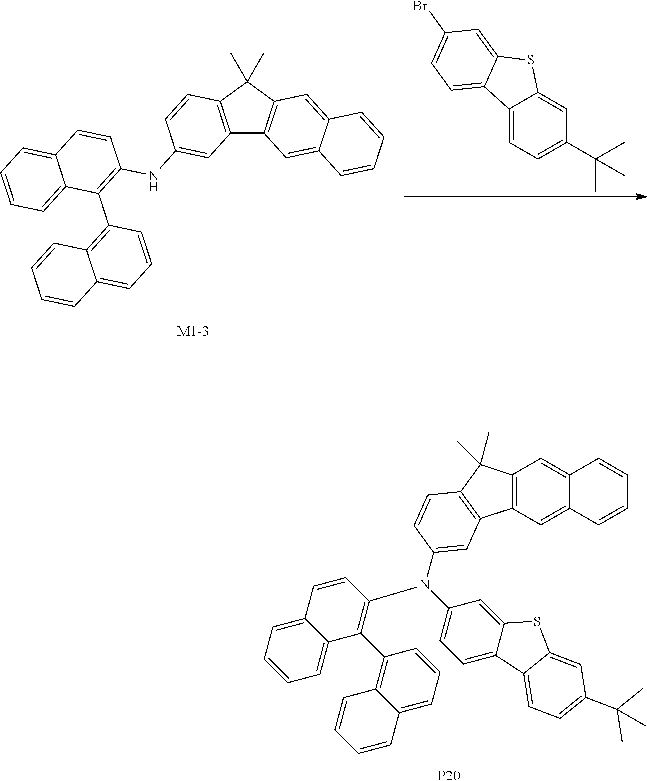

Into a 1000 mL single-necked bottle, 25.5 g (50 mmol) of M1-3, 16 g (50 mmol) of 3-bromo-6-tert-butyldibenzothiophene, 0.9 g (1 mmol) of tris(dibenzylideneacetone)dipalladium (i.e. Pd₂(dba)₃), 0.5 mL of Tri-tert-butylphosphine((t-Bu)₃P), 500 mL of toluene, and 14.4 g (150 mmol) of sodium tert-butoxide(NaOBu-t) were added, vacuumized and replaced with nitrogen for three times, and the reaction was heated up to 110° C. for reacting 5 hours. After the reaction was completed, it was stopped. The reaction was cooled to room temperature, the reaction solution was layered, the organic phase was concentrated and methanol was added and stirred for 1 hour, suction filtered to obtain P20 as a light yellow powder.

M/Z theoretical value: 749; ZAB-HS Model Mass Spectrometer (Manufactured by Micromass, British) M/Z found: 750.

### Synthesis Example 16: Synthesis of Compound P628

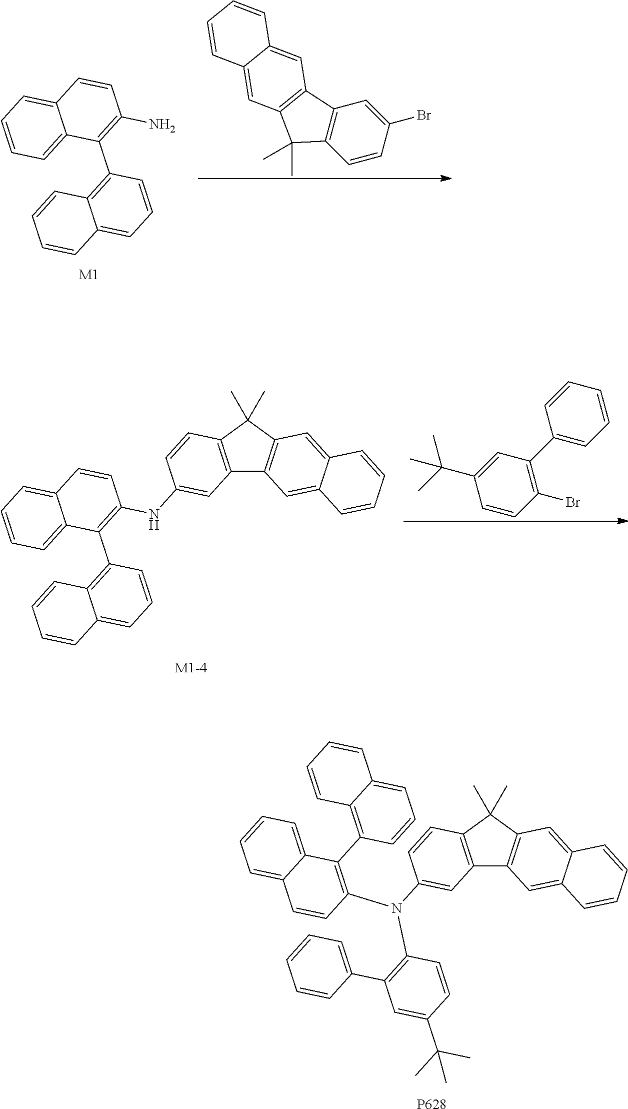

Into a 1000 mL single-necked bottle, 13.5 g (50 mmol) of M1, 16.5 g (50 mmol) of 3-bromo-9,9-dimethyl-5,6-benzofluorene, 0.9 g (1 mmol) of tris(dibenzylideneacetone)dipalladium (i.e. Pd₂(dba)₃), 0.5 g of IPr·HCl, 500 mL of toluene, and 14.4 g (150 mmol) of sodium tert-butoxide(NaOBu-t) were added, vacuumized and replaced with nitrogen for three times, and the reaction was heated up to 90° C. for reacting 5 hours. After the reaction was completed, it was stopped. The reaction was cooled to room temperature, the reaction solution was layered, the organic phase was concentrated and methanol was added and stirred for 1 hour, suction filtered to obtain M1-4 as a light yellow powder.

Into a 1000 mL single-necked bottle, 25.5 g (50 mmol) of M1-4, 14.5 g (50 mmol) of 2-bromo-5-tert-butylbiphenyl, 0.9 g (1 mmol) of tris(dibenzylideneacetone)dipalladium (i.e. Pd₂(dba)₃), 0.5 mL of Tri-tert-butylphosphine((t-Bu)₃P), 500 mL of toluene, 14.4 g (150 mmol) of sodium tert-butoxide(NaOBu-t) were added, vacuumized and replaced with nitrogen for three times, and the reaction was heated up to 110° C. for reacting 5 hours. After the reaction was completed, it was stopped. The reaction was cooled to room temperature, the reaction solution was layered, the organic phase was concentrated and methanol was added and stirred for 1 hour, suction filtered to obtain P628 as a light yellow powder.

M/Z theoretical value: 719; ZAB-HS Model Mass Spectrometer (Manufactured by Micromass, British) M/Z found: 720.

### Synthesis Example 17: Synthesis of Compound P613

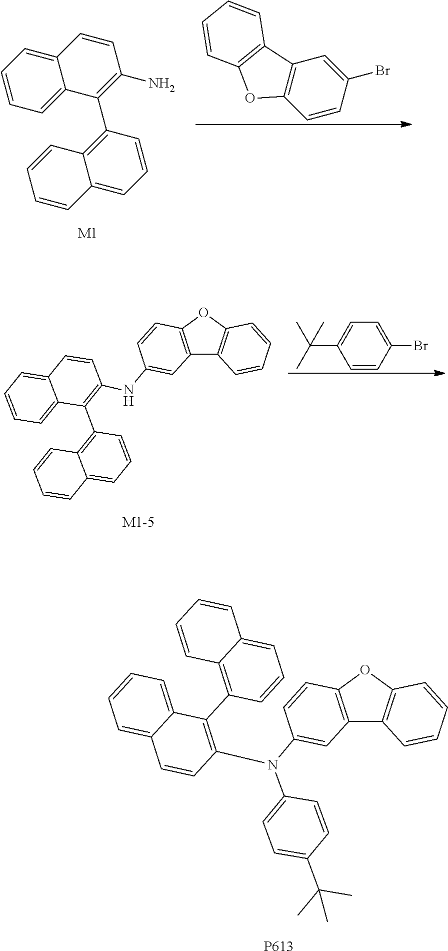

Into a 1000 mL single-necked bottle, 13.5 g (50 mmol) of M1, 12.3 g (50 mmol) of 3-bromo-dibenzofuran, 0.9 g (1 mmol) of tris(dibenzylideneacetone)dipalladium (i.e. Pd₂(dba)₃), 0.5 g of IPr·HCl, 500 mL of toluene, and 14.4 g (150 mmol) of sodium tert-butoxide(NaOBu-t) were added, vacuumized and replaced with nitrogen for three times, and the reaction was heated up to 90° C. for reacting 5 hours. After the reaction was completed, it was stopped. The reaction was cooled to room temperature, the reaction solution was layered, the organic phase was concentrated and methanol was added and stirred for 1 hour, suction filtered to obtain M1-5 as a light yellow powder.

Into a 1000 mL single-necked bottle, 22 g (50 mmol) of M1-5, 11 g (50 mmol) of 4-bromo-4′-tert-butylbiphenyl, 0.9 g (1 mmol) of tris(dibenzylideneacetone)dipalladium (i.e. Pd₂(dba)₃), 0.5 mL of Tri-tert-butylphosphine ((t-Bu)₃P), 500 mL of toluene, and 14.4 g (150 mmol) of sodium tert-butoxide (NaOBu-t) were added, vacuumized and replaced with nitrogen for three times, and the reaction was heated up to 110° C. for reacting 5 hours. After the reaction was completed, it was stopped. The reaction was cooled to room temperature, the reaction solution was layered, the organic phase was concentrated and methanol was added and stirred for 1 hour, suction filtered to obtain P613 as a light yellow powder.

M/Z theoretical value: 567; ZAB-HS Model Mass Spectrometer (Manufactured by Micromass, British) M/Z found: 568.

### Synthesis Example 18: Synthesis of Compound P602

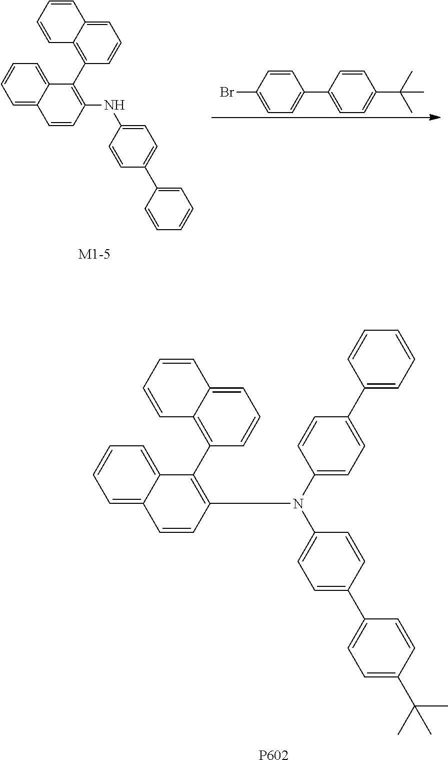 [SMILES: c1ccc(-c2ccc(Nc3ccc4ccccc4c3-c3cccc4ccccc34)cc2)cc1 ; CC(C)(C)c1ccc(-c2ccc(Br)cc2)cc1 ; CC(C)(C)c1ccc(-c2ccc(N(c3ccc(-c4ccccc4)cc3)c3ccc4ccccc4c3-c3cccc4ccccc34)cc2)cc1 ; "P602"]

Into a 1000 mL single-necked bottle, 21 g (50 mmol) of M1-5, 14.5 g (50 mmol) of 4-bromo-4′-tert-butylbiphenyl, 0.9 g (1 mmol) of tris(dibenzylideneacetone)dipalladium (i.e. Pd₂(dba)₃), 0.5 mL of Tri-tert-butylphosphine((t-Bu)₃P), 500 mL of toluene, and 14.4 g (150 mmol) of sodium tert-butoxide(NaOBu-t) were added, vacuumized and replaced with nitrogen for three times, and the reaction was heated up to 110° C. for reacting 5 hours. After the reaction was completed, it was stopped. The reaction was cooled to room temperature, the reaction solution was layered, the organic phase was concentrated and methanol was added and stirred for 1 hour, suction filtered to obtain P602 as a light yellow powder.

M/Z theoretical value: 629; ZAB-HS Model Mass Spectrometer (Manufactured by Micromass, British) M/Z found: 630.

### Example 1

This example provides an organic electroluminescent device. The specific preparation process is as follows:

After ultrasonic treatment of the glass plate coated with ITO transparent conductive layer (as anode) in a commercial cleaning agent, it was rinsed in deionized water, the oil was removed by ultrasonic in a mixed solvent of acetone and ethanol, baked in a clean environment until the water was completely removed, cleaned with ultraviolet light and ozone, and the surface was bombarded with a low-energy cation beam;

the above glass substrate with anode was placed in the vacuum chamber and vacuumized to less than 1×10⁻⁵ Pa, HI-3 was vacuum evaporated on the aforementioned anode layer film as a hole injection layer, the evaporation rate was 0.1 nm/s, and the evaporation film thickness was 10 nm;

HT-4 was vacuum evaporated on the hole injection layer as the hole transporting layer of the device, the evaporation rate was 0.1 nm/s, and the total evaporation film thickness was 80 nm;

Compound P1 was vacuum evaporated on the hole transporting layer as the electron blocking layer material of the device, the evaporation rate was 0.1 nm/s, and the total evaporation film thickness was 80 nm.

A light-emitting layer of the device was vacuum evaporated on the top of the electron blocking layer, the light emitting layer included a host material and a dye material. Using the method of mufti-source co-evaporation, the evaporation rate of the host material GPH-59 was adjusted to 0.1 nm/s, and the evaporation rate of the dye RPD-8 of 3% of the host material was proportionally set, and the total evaporation film thickness was 30 nm;

the electron transporting layer material ET-46 of the device was vacuum evaporated on the light emitting layer, the evaporation rate thereof was 0.1 nm/s, and the total evaporation film thickness was 30 nm;

LiF with a thickness of 0.5 nm was vacuum evaporated on the electron transporting layer (ETL) as the electron injection layer, and Al layer with a thickness of 150 nm was used as the cathode of the device.

In examples 2 to 18, the manufacturing process of the organic electroluminescent devices provided in the comparative examples 1 to 6 is the same as that in example 1, with the difference lying in that the compound P1 of the electron blocking layer material is replaced with the compound as shown in Table 1, respectively.

The structure of the electron blocking layer material in comparative examples 1 to 6 is as follows:

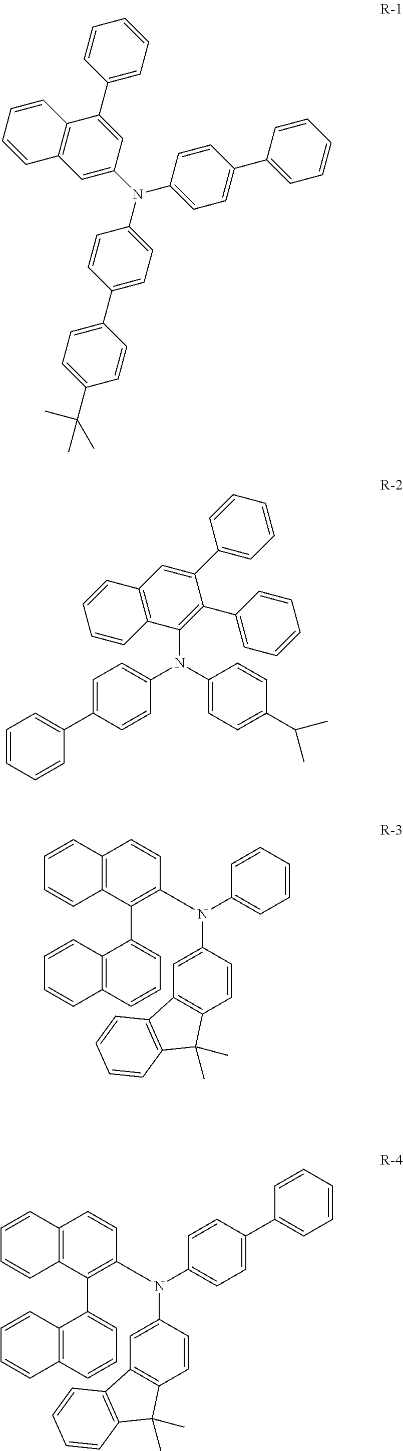

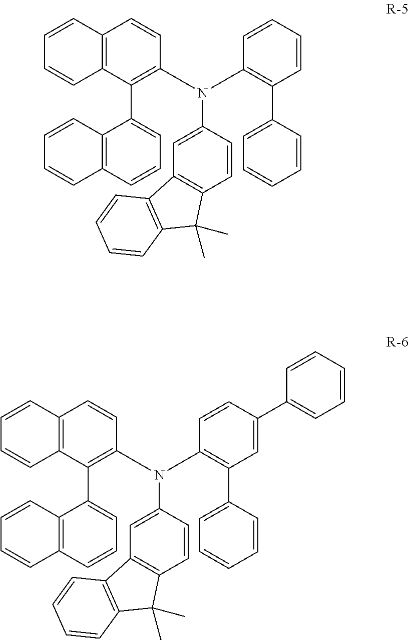

wherein, see compound 39 and compound 94 in patent application CN109749735A for R-1 and R-2, and see compound P405, P389, P406 and compound P418 in patent application CN110511151A for R-3 to R-6.

The performance of the organic electroluminescent device prepared by the above mentioned process is measured as follows:

Under the same brightness, the driving voltage and current efficiency of the organic electroluminescent devices prepared in examples 1 to 18 and comparative examples 1 to 6 were measured with a digital source meter (Keithley 2400), a luminance meter (ST-86LA type luminance meter, Optoelectronic Instrument Factory of Beijing Normal University) and a luminance meter. Specifically, the voltage was increased at a rate of 0.1V per second, the voltage when the brightness of the organic electroluminescent device reached 3000 cd/m² was measured, that is, the driving voltage, at which time the current density was measured; and the ratio of brightness to current density was the current efficiency. See Table 1 for the test results.

TABLE 1

| | electron blocking | | current |
|---|---|---|---|
| | layer material | voltage/V | efficiency/cd/A |
| Comparative example 1 | R-1 | 5.3 | 11 |
| Comparative example 2 | R-2 | 5.8 | 10.1 |
| Comparative example 3 | R-3 | 3.3 | 19.0 |
| Comparative example 4 | R-4 | 3.1 | 19.3 |
| Comparative example 5 | R-5 | 3.4 | 18.5 |
| Comparative example 6 | R-6 | 3.5 | 17.8 |
| Example 1 | P1 | 3.3 | 19.5 |
| Example 2 | P2 | 3.2 | 20.1 |
| Example 3 | P5 | 3.4 | 19.0 |
| Example 4 | P6 | 3.5 | 18.2 |
| Example 5 | P7 | 3.4 | 19.3 |
| Example 6 | P15 | 3.3 | 19.4 |
| Example 7 | P20 | 3.4 | 18.7 |
| Example 8 | P37 | 3.2 | 19.8 |
| Example 9 | P38 | 3.1 | 20.3 |
| Example 10 | P74 | 3.3 | 19.6 |
| Example 11 | P253 | 3.2 | 19.7 |
| Example 12 | P284 | 3.5 | 19.5 |
| Example 13 | P322 | 3.6 | 19.4 |
| Example 14 | P323 | 3.6 | 19.5 |
| Example 15 | P375 | 3.5 | 20.0 |
| Example 16 | P613 | 3.4 | 19.9 |
| Example 17 | P628 | 3.3 | 18.9 |
| Example 18 | P602 | 3.5 | 18.4 |

It can be seen from the data in Table 1 that when the compound of the disclosure is used for the electron blocking layer material of the organic electro luminescent device, when the device brightness reaches 3000 cd/m², the driving voltage is as low as 3.8 V or less, and the current efficiency is as high as 18.2 cd/A or more, which can effectively improve the driving voltage and current efficiency, and is an electron blocking layer material with good performance.

Compared the compound R-1 of comparative example 1 with the compound P602 of example 18, the difference lies in that in the structure of compound R-1, there is no substitution at 1-position of naphthalene and there is a benzene ring substitution at 4-position. When the compound is used as the electron blocking layer material of the organic electroluminescent device, the driving voltage of the devices is 5.3 V and the current efficiency is 11 cd/A. The starting voltage and current efficiency of the compound are lower than those of P602, which may be attributed to the better space packing of compound P602, which improves the hole transporting properties.

The arylamino group of compound R-2 in comparative example 2 is substituted on the 1-position of the naphthalene ring, and the benzone ring is substituted on the 2-position and the 3-position, without containing the substituents of the tert-butyl structure. When the compound is used as the electron blocking layer material of the organic electroluminescent device, the driving voltage of the device is 5.8V, the current efficiency is 10.1 cd/A, and the effect is significantly worse than that of examples 1 to 18.

Compared the compound R-3 of comparative example 3 with the compound P1 of example 1, the difference lies in that there is no tert-butyl substitution at the 4-position of the phenyl group connected with N. When the compound is used as the electron blocking layer material of the organic electroluminescent device, the driving voltage of the device is 3.3 V, and the current efficiency is 19 cd/A. The current efficiency of the compound is lower than that of P1, which may be attributed to the fact that the tert-butyl at the 4-position in compound P1 can not only provide strong electron donating ability, but also improve the molecular space packing structure, thus effectively improving the hole transporting performance of the material.

Compared the compound R-4 of comparative example 4 with the compound P2 of example 2, the difference lies in that there is no tert-butyl substitution at the biphenyl end connected with N in the molecule. When the compound is used as the electron blocking layer material of the organic electroluminescent device, the driving voltage of the device is 3.1V, and the current efficiency is 19.3 cd/A. The current efficiency of the compound is lower than that of P2, which may be attributed to the fact that the tert-butyl at the 4-position in compound P2 can not only provide electron donating ability, but also improve the molecular space packing structure, thus effectively improving the hole transporting performance of the material.

Compared the compound R-5 of comparative example 5 with the compound P5 of example 3, the difference lies in that there is no tert-butyl substitution at the 4-position of the biphenyl group connected with N. When the compound is used as the electron blocking layer material of the organic electroluminescent device, the driving voltage of the device is 3.4V, and the current efficiency is 18.5 cd/A. The current efficiency of the compound is lower than that of P5, which may be attributed to the fact that the tert-butyl at the 4-position in compound P5 can not only provide strong electron donating ability, but also improve the molecular space packing structure, thus effectively improving the hole transporting performance of the material.

Compared the compound R-6 of comparative example 6 with the compound P6 of example 4, the difference lies in that there is no tert-butyl substitution at the 2-phenylbiphenyl end connected with N in the molecule. When the compound is used as the electron blocking layer material of the organic electroluminescent device, the driving voltage of the device is 3.5V, and the current efficiency is 17.8 cd/A. The current efficiency of the compound is lower than that of P6, which may be attributed to the fact that the tert-butyl in compound P6 compound can not only provide electron donating ability, but also improve the molecular space packing structure, thus effectively improving the hole transporting performance of the material.

It can be seen that in the compounds provided by the disclosure, the substituent Ar² at 1-position of the naphthalene ring, the substituent arylamino group at 2-position as well as the tert-butyl structure substituent are important factors that enable the compounds to bring excellent performances when applied to the organic electroluminescent devices.

### Example 19

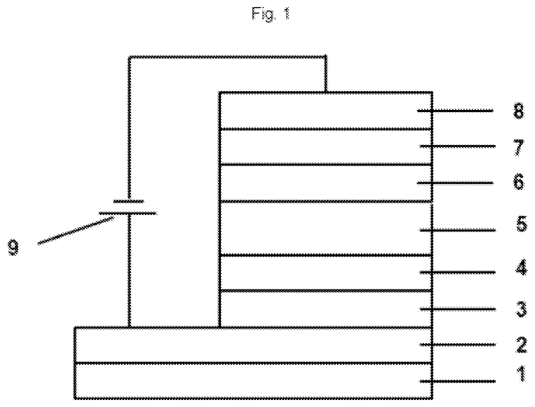

This example provides an organic electroluminescent device, whose structure is as shown in FIG. 1, including a glass substrate 1 with an anode, a hole injection layer 2, a hole transporting layer 3, an electron blocking layer 4, a light emitting layer 5, an electron transporting layer 6, an electron injection layer 7, a cathode layer 8 and an external power supply 9.

The preparation method of the organic electroluminescent devices is as follows:

After ultrasonic treatment of the glass plate coated with ITO transparent conductive layer in a commercial cleaning agent, it was rinsed in deionized water, the oil was removed by ultrasonic in a mixed solvent of acetone and ethanol, baked in a clean environment until the water was completely removed, cleaned with ultraviolet light and ozone, and the surface was bombarded with a low-energy cation beam;

the glass substrate with an anode was placed in the vacuum chamber and vacuumized to less than 1×10⁻⁵ Pa, the HT-4:HI-3 (97/3, w/w) mixture of 10 nm was vacuum hot evaporated on the aforementioned anode layer film as the hole injection layer: 60 nm of compound HT-4 as the hole transporting layer; 5 nm of compound HT-48 as the electron blocking layer; 40 nm of PH-34:P1:RPD-10 (100:30:3, w/w/w) ternary mixture as the light emitting layer; 5 nm of ET-23 as the hole blocking layer, 25 nm of compound ET-69: ET-57 (50/50, w/w) mixture as the electron transporting layer, 1 nm of LiF as the electron injection layer, and 150 nm of the metal aluminum as the cathode in sequence. The total evaporation rate of all organic layers and LiF is controlled at 0.1 nm/s, and the evaporation rate of metal electrode is controlled at 1 nm/s. wherein, “97/3, w/W” represents the mass ratio of 97:3.

The differences among examples 19-42, comparative examples 7-8 and example 19 are listed in Table 3, respectively, and the parts not mentioned in Table 3 are the same as those in example 19.

Performance Testing

(1) The HOMO energy level and LUMO energy level of the second host material used in the previous examples and comparative examples are shown in Table 2 in details.

TABLE 2

| | HOMO energy level (eV) | LUMO energy level (eV) |
|---|---|---|
| PH-34 | −5.4 eV | −2.4 eV |
| PH-27 | −5.2 eV | −2.2 eV |

(2) Under the same brightness, the current efficiencies of the organic electroluminescent devices prepared in the examples and the comparative examples were measured. Specifically, the voltage was increased at a rate of 0.1 V per second, the current density when the brightness of the organic electroluminescent device reached 3000 cd/m² was measured; and the ratio of brightness to current density was the current efficiency. The test results are shown in Table 3.

TABLE 3

| | Host materials of the | Thickness of | | |
|---|---|---|---|---|
| | light emitting layer | the light | Required | Current |
| | and proportions | emitting layer | brightness | efficiency |
| Compound No. | (w/w) | nm | cd/m² | cd/A |
| Comparative | PH-34 | 40 | 3000 | 10.36 |
| example 7 | | | | |
| Comparative | P1 | 40 | | 10.11 |
| example 8 | | | | |
| Example 19 | P1:PH-34 = 30:100 | 40 | | 17.68 |
| Example 20 | P1:PH-34 = 80:100 | 40 | | 15.73 |
| Example 21 | P1:PH-34 = 30:100 | 50 | | 15.21 |
| Example 22 | P1:PH-34 = 1:100 | 40 | | 15.47 |
| Example 23 | P1:PH-34 = 150:100 | 40 | | 15.08 |
| Example 24 | P1:PH-34 = 10:100 | 40 | | 17.29 |
| Example 25 | P1:PH-34 = 100:100 | 40 | | 17.81 |
| Example 26 | P1:PH-34 = 0.9:100 | 40 | | 11.7 |
| Example 27 | P1:PH-34 = 160:100 | 40 | | 13 |
| Example 28 | P1:PH-34 = 30:100 | 10 | | 15.86 |
| Example 29 | P1:PH-34 = 30:100 | 65 | | 15.47 |
| Example 30 | P1:PH-34 = 30:100 | 15 | | 16.9 |
| Example 31 | P1:PH-34 = 30:100 | 55 | | 17.03 |
| Example 32 | P1:PH-34 = 30:100 | 9 | | 12.74 |
| Example 33 | P1:PH-34 = 30:100 | 66 | | 12.87 |
| Example 34 | P1:PH-27 = 30:100 | 40 | | 14.56 |
| Example 35 | P12:PH-34 = 30:100 | 40 | | 16.38 |
| Example 36 | P23:PH-34 = 30:100 | 40 | | 16.9 |
| Example 37 | P25:PH-34 = 30:100 | 40 | | 15.34 |
| Example 38 | P56:PH-34 = 30:100 | 40 | | 16.64 |
| Example 39 | P68:PH-34 = 30:100 | 40 | | 16.25 |
| Example 40 | P107:PH-34 = 30:100 | 40 | | 15.86 |
| Example 41 | P142:PH-34 = 30:100 | 40 | | 17.03 |
| Example 42 | P509:PH-34 = 30:100 | 40 | | 15.6 |

The comparative example 7 is a single host device, where the mass ratio of PH-34 to RPD-10 is 130:3; the comparative example 8 is also a single host device, where the mass ratio of P1 to RPD-10 is 130:3.

It can be seen from Table 3 that the organic electroluminescent device containing the dual host light emitting layer provided by the disclosure has excellent photoelectric performance, and its current efficiency is 11.7 cd/A or more, most of which can reach 15 cd/A or more, and the maximum can reach 17 cd/A or more. The single host device is adopted for the comparative examples 7 and 8, and the effect is obviously inferior to that of the disclosure.

By comparing examples 19 and 22-27, it can be seen that when the mass ratio of the first host material to the second host material is 0.01:1-1.5:1 (examples 19, 22-27), the device efficiency can be further improved, where the effect is the best at 0.1:1-1:1 (examples 19, 24 and 25).

By comparing examples 19 and 28-33, it can be seen that when the thickness of the dual host light emitting layer is 10-65 nm (examples 19, 28-31), the device efficiency can be further improved, where the effect is the best when the thickness is within the range of 15-55 nm (examples 19, 30 and 31).

It can be seen from the comparison between example 19 and example 34 that when the second host material meets the specific LUMO energy level and HOMO energy level (example 19), it is beneficial to further improve the device efficiency.

In the following examples, the synthesis method of compound I is as described above. For the synthesis method of compound II, reference can be made to the Chinese patent application with the publication number CN110950762A (application number 201910857132.9).

### Example 43

This example provides an organic electroluminescent device, whose structure is as shown in FIG. 1, including a glass substrate 1 with an anode, a hole injection layer 2, a hole transporting layer 3, an electron blocking layer 4, a light emitting layer 5, an electron transporting layer 6, an electron injection layer 7, a cathode layer 8 and an external power supply 9.

The preparation method of the organic electroluminescent devices is as follows:

After ultrasonic treatment of the glass plate coated with ITO transparent conductive laver in a commercial cleaning agent, it was rinsed in deionized water, the oil was removed by ultrasonic in a mixed solvent of acetone and ethanol, baked in a clean environment until the water was completely removed, cleaned with ultraviolet light and ozone, and the surface was bombarded with a low-energy cation beam;

the glass substrate with an anode was placed in the vacuum chamber and vacuumized to less than 1×10⁻⁵ Pa, the HT-4:HI-3 (97/3, w/w) mixture of 10 nm was vacuum hot evaporated on the aforementioned anode layer film as the hole injection layer; 60 nm of compound HT-4 as the hole transporting layer; 60 nm of compound A1 as the electron blocking layer; 30 nm of compound PH86:P1:RPD-10 (1:0.01:0.05, w/w/w) ternary mixture as the light emitting layer (wherein, PH86 and P1 were the host materials): 25 nm of compound ET-61:ET-57(50/50, w/w) mixture as the electron transporting layer, 1 nm of LiF as the electron injection layer, and 150 nm of the metal aluminum as the cathode in sequence. The total evaporation rate of all organic layers and LiF is controlled at 0.1 nm/s, and the evaporation rate of metal electrode is controlled at 1 nm/s.

The differences among examples 44-68, comparative examples 9-12 and example 43 are all listed in Table 4, and the parts not mentioned in Table 4 are the same as those in example 43.

Performance Testing

Under the same brightness, the external quantum efficiency (EQE, %) of the organic electroluminescent devices prepared in the examples and the comparative examples were measured, the required brightness is 3000 cd/m².

TABLE 4

| | | | Thickness of | Thickness | |
|---|---|---|---|---|---|
| | Light emitting | | light emitting | of electron | |
| | layer material | electron blocking | layer | blocking layer | |
| | and proportions | layer material | (nm) | (nm) | EQE % |
| Comparative | PH86 | HT5 | 30 | 60 | 12.76 |
| example 9 | | | | | |
| Comparative | PH86:A1 = 1:0.3 | HT5 | 30 | 60 | 11.98 |
| example 10 | | | | | |
| Comparative | PH86:P1 = 1:0.3 | HT5 | 30 | 60 | 12.66 |
| example 11 | | | | | |
| Comparative | PH86:HT-10 = 1:0.3 | A1 | 30 | 60 | 11.87 |
| example 12 | | | | | |
| Example 43 | PH86:P1 = 1:0.01 | A1 | 30 | 60 | 15.33 |
| Example 44 | PH86:P1 = 1:0.1 | A1 | 30 | 60 | 16.67 |
| Example 45 | PH86:P1 = 1:0.3 | A1 | 30 | 60 | 19.63 |
| Example 46 | PH86:P1 = 1:1 | A1 | 30 | 60 | 19.22 |
| Example 47 | PH86:P1 = 1:1.5 | A1 | 30 | 60 | 18.58 |
| Example 48 | PH86:P1 = 1:0.3 | A1 | 10 | 60 | 18.96 |
| Example 49 | PH86:P1 = 1:0.3 | A1 | 20 | 60 | 19.10 |
| Example 50 | PH86:P1 = 1:0.3 | A1 | 50 | 60 | 18.92 |
| Example 51 | PH86:P1 = 1:0.3 | A1 | 60 | 60 | 18.23 |
| Example 52 | PH86:P1 = 1:0.3 | A1 | 30 | 2 | 15.49 |
| Example 53 | PH86:P1 = 1:0.3 | A1 | 30 | 3 | 16.03 |
| Example 54 | PH86:P1 = 1:0.3 | A1 | 30 | 90 | 18.85 |
| Example 55 | PH86:P1 = 1:0.3 | A1 | 30 | 100 | 17.98 |
| Example 56 | PH86:A1 = 1:0.3 | P1 | 30 | 60 | 18.18 |
| Example 57 | P1:RPD-10 = 1:5 | A1 | 30 | 60 | 14.42 |
| Example 58 | A1:RPD-10 = 0.3:0.05 | P1 | 30 | 60 | 14.64 |
| Example 59 | PH86:P1 = 1:0.005 | A1 | 30 | 60 | 13.28 |
| Example 60 | PH86:P1 = 1:1.51 | A1 | 30 | 60 | 13.87 |
| Example 61 | PH86:P1 = 1:0.3 | A1 | 9 | 60 | 14.10 |
| Example 62 | PH86:P1 = 1:0.3 | A1 | 61 | 60 | 14.55 |
| Example 63 | PH86:P1 = 1:0.3 | A1 | 9 | 1 | 13.98 |
| Example 64 | PH86:P1 = 1:0.3 | A1 | 30 | 101 | 13.87 |
| Example 65 | PH86:P2 = 1:0.3 | A3 | 30 | 60 | 16.28 |
| Example 66 | PH86:P5 = 1:0.3 | A8 | 30 | 60 | 15.46 |
| Example 67 | PH86:P6 = 1:0.3 | A35 | 30 | 60 | 15.89 |
| Example 68 | PH86:P32 = 1:0.3 | A56 | 30 | 60 | 16.00 |

It can be seen from Table 4 that compound I and compound II are used in organic electroluminescent devices in the disclosure, which can effectively improve the external quantum efficiency, thereby improving the device performance, and ultimately exhibiting excellent characteristics such as reduced device energy consumption, and improved brightness, etc., they are electron blocking layer and light emitting layer materials with good performances, and the external quantum efficiency of the device can reach 19%.

The difference between comparative example 10 and example 56 only lies in that the electron blocking layer material is HT5, and its external quantum efficiency is significantly lower than that of example 14; The difference between comparative example 11 and example 45 only lies in that the electron blocking layer material is HT5, and its external quantum efficiency is significantly lower than that of example 45; The difference between comparative example 4 and example 45 only lies in that the host material is PH86 and HT-10, and its external quantum efficiency is significantly reduced relative to example 45; The above results prove that the combination of compound I and compound II in the disclosure can effectively improve the device efficiency, and replacing any one of them will reduce the efficiency.

By comparing example 45 with example 57, example 56 and example 58, it can be seen that when compound I or compound II is applied to the dual host light emitting layer, the device efficiency can be further improved (example 45, and example 56), and the effect becomes worse when used alone (example 57, and example 58).

By comparing examples 43-47, 59 and 60, it can be seen that when the mass ratio of the first host material to the second host material is 0.01:1-1.5:1 (examples 43-47), the external quantum efficiency can be further improved. Too low (example 59) or too high (example 60) of the addition amount will reduce the efficiency.

By comparing examples 45, 48-51, 61, and 62, it can be seen that controlling the thickness of the light emitting layer at 10-60 nm (examples 45, 48-51) can further improve the external quantum efficiency of the device. If the thickness is too small (example 61) or too large (examples 62), the efficiency will be reduced.

By comparing examples 45, 52-55 and 63, 64, it can be seen that controlling the thickness of the electron blocking layer at 2-100 nm (examples 45, 52-55) can further improve the external quantum efficiency of the device. If the thickness is too small (example 63) or too large (examples 64), the efficiency will be reduced.

The above descriptions are only the preferred examples of the present invention, and is not intended to limit thereto. For those skilled in the art, various modifications and changes can be made to the present invention. Any modifications, equivalent substitutions, improvements, and the like made within the spirit and principle of the present invention shall be included into the protection scope of the present invention.

1. A compound, wherein, the compound has a structure as shown in Formula I; 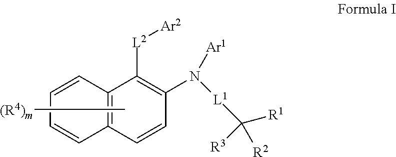
In Formula I, the Ar¹ and Ar² are independently selected from the group consisting of a substituted or unsubstituted C6-C30 aryl group or a substituted or unsubstituted C3-C30 heteroaryl group;
In Formula I, the L¹ is selected from the group consisting of a substituted or unsubstituted C6-C30 arylene or a substituted or unsubstituted C3-C30 heteroarylene;
In Formula I, the L² is selected from one of a single bond, a substituted or unsubstituted C6-C30 arylene or a substituted or unsubstituted C3-C30 heteroarylene;
In Formula I, the R¹, R² and R³ are independently selected from the group consisting of any one of a substituted or unsubstituted C1-C20 chain alkyl, a substituted or unsubstituted C3-C20 cycloalkyl, a substituted or unsubstituted C1-C20 chain alkoxy, a substituted or unsubstituted C3-C20 cycloalkoxy, a substituted or unsubstituted C2-C10 alkenyl, a substituted or unsubstituted C2-C10 alkynyl, halogen, cyano, nitro, hydroxyl, C1-C20 silyl, amino, a substituted or unsubstituted C6-C30 arylamino, a substituted or unsubstituted C3-C30 heteroarylamino, a substituted or unsubstituted C6-C30 aryl, a substituted or unsubstituted C3-C30 heteroaryl; the R¹, R² and R³ are connected to form a ring or not connected to form a ring;
In Formula I, the m is an integer from 0 to 6;
In Formula I, the R⁴ is independently selected from the group consisting of one of a substituted or unsubstituted C1-C20 chain alkyl, a substituted or unsubstituted C3-C20 cycloalkyl, a substituted or unsubstituted C1-C20 chain alkoxy, a substituted or unsubstituted C3-C20 cycloalkoxy, a substituted or unsubstituted C2-C10 alkenyl, a substituted or unsubstituted C2-C10 alkynyl, halogen, cyano, nitro, hydroxyl, a substituted or unsubstituted C1-C20 silyl, amino, a substituted or unsubstituted C6-C30 arylamino, a substituted or unsubstituted C3-C30 heteroarylamino, a substituted or unsubstituted C6-C30 aryl, a substituted or unsubstituted C3-C30 heteroaryl;
In Ar¹, Ar², L¹, L², R¹, R², R³ and R⁴, the substituted groups are each independently selected from the group consisting of one or a combination of at least two of halogen, C1-C10 chain alkyl, C3-C10 cycloalkyl, C1-C10 alkoxy, C1-C10 thioalkoxy, C6-C30 arylamino, C3-C30 heteroarylamino, C6-C30 monocyclic aryl, C10-C30 fused ring aryl, C3-C30 monocyclic heteroaryl, C6-C30 fused ring heteroaryl.

2. The compound according to claim 1, wherein, the L² is selected from the group consisting of one of a single bond, a substituted or unsubstituted C6-C20 arylene or a substituted or unsubstituted C3-C20 heteroarylene, preferably a single bond or phenylene.

3. The compound according to claim 1, wherein, the Ar² is selected from the group consisting of a substituted or unsubstituted C6-C20 aryl or a substituted or unsubstituted C3-C20 heteroaryl;
preferably, the Ar² is selected from the group consisting of any of the following substituted or unsubstituted groups: phenyl, biphenyl, terphenyl, naphthyl, phenanthryl, anthracyl, dibenzofuranyl, dibenzothiophenyl, carbazolyl, 9,9 dimethylfluorenyl, 9,9 diphenylfluorenyl, spirofluorenyl, triphenylene, fluoranthenyl, benzo9,9 dimethylfluorenyl, and benzospirofluorenyl;
preferably, the Ar² is selected from the group consisting of any of the following substituted or unsubstituted groups: 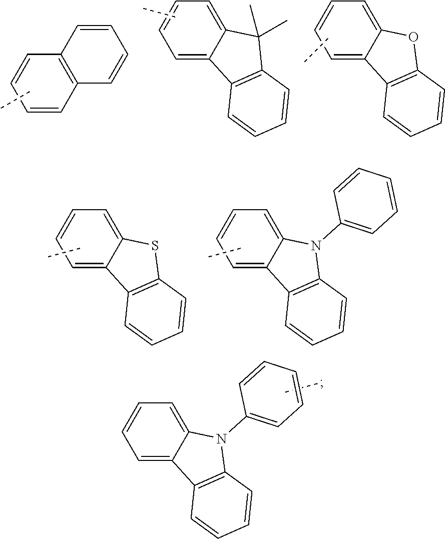
wherein, the dotted line represents a connecting bond of the groups.

4. (canceled)

5. The compound according to claim 1, wherein, the L² is a single bond, and the Ar² is selected from the group consisting of a substituted or unsubstituted C10-C30 fused ring aryl or a substituted or unsubstituted C6-C30 fused ring heteroaryl;
preferably, the L² is a single bond, and the Ar² is selected from the group consisting of any of the following substituted or unsubstituted groups: naphthyl, phenanthryl, anthracyl, dibenzofuranyl, dibenzothiophenyl, carbazolyl, 9,9dimethylfluorenyl, 9,9diphenylfluorenyl, spirofluorenyl, triphenylene, fluoranthenyl, benzo9,9dimethylfluorenyl, and benzospirofluorenyl;
preferably, the L² is a single bond, and the Ar² is selected from the group consisting of any of the following substituted or unsubstituted groups: 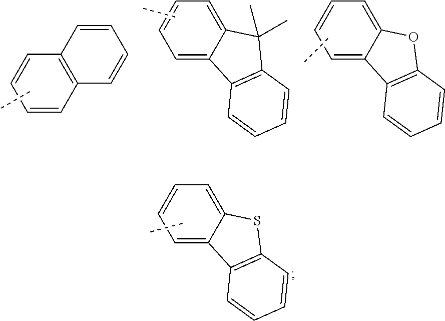
wherein, the dotted line represents a connecting bond of the groups;
preferably, the L² is the phenylene, and the Ar² is selected from the group consisting of a substituted or unsubstituted C6-C30 aryl or a substituted or unsubstituted C3-C30 heteroaryl;
preferably, the L² is the phenylene, and the Ar² is selected from the group consisting of any of the following substituted or unsubstituted groups: phenyl, biphenyl, terphenyl, naphthyl, phenanthryl, anthracyl, dibenzofuranyl, dibenzothiophenyl, carbazolyl, 9,9dimethylfluorenyl, 9,9diphenylfluorenyl, spirofluorenyl, triphenylene, fluoranthenyl, benzo9,9dimethylfluorenyl, and benzospirofluorenyl;
preferably, the L² is the phenylene, and the Ar² is selected from the group consisting of any of the following substituted or unsubstituted groups: 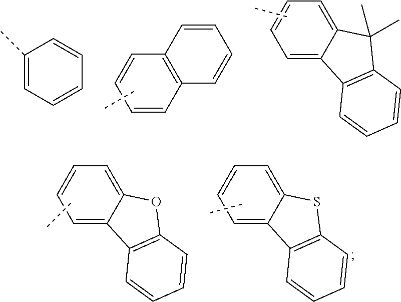
wherein, the dotted line represents a connecting bond of the groups.

6. (canceled)

7. The compound according to claim 1, wherein, the R¹, R² and R³ are independently selected from the group consisting of one of methyl, ethyl, or phenyl;
preferably, the R¹, R² and R³ are all methyl;
preferably, the L¹ is selected from the group consisting of one of the following substituted or unsubstituted groups: phenylene, biphenylene, naphthylene, dibenzofuranylene, dibenzothiophenylene, and 9,9dimethylfluorenylene;
preferably, the L¹ is selected from the group consisting of any of the following substituted or unsubstituted groups: 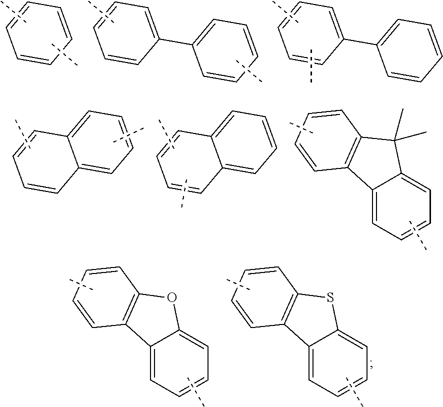
wherein, the dotted line represents a connecting bond of the groups;
preferably, the m is 0.

8. (canceled)

9. (canceled)

10. (canceled)

11. The compound according to claim 1, wherein, the compound has any one of the following structures as shown in P1-P777:

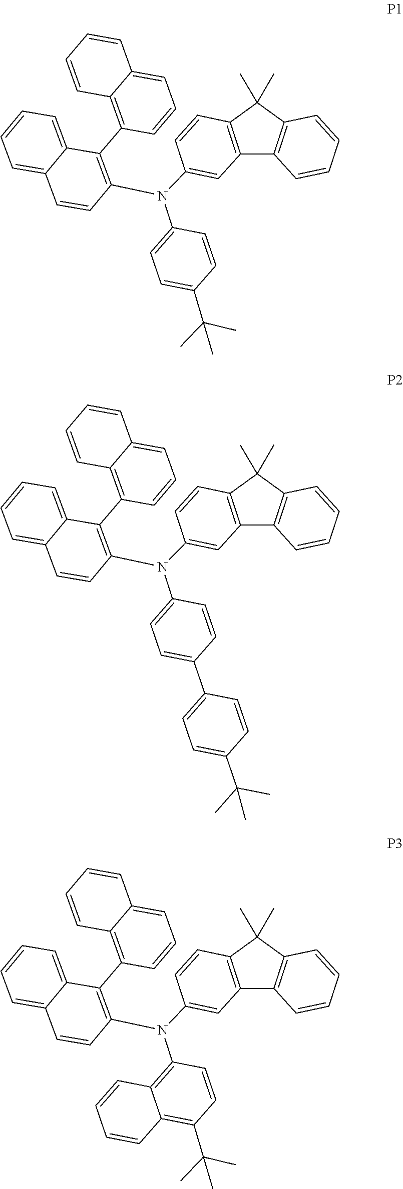 [SMILES: CC(C)(C)c1ccc(N(c2ccc3c(c2)-c2ccccc2C3(C)C)c2ccc3ccccc3c2-c2cccc3ccccc23)cc1 ; CC(C)(C)c1ccc(-c2ccc(N(c3ccc4c(c3)-c3ccccc3C4(C)C)c3ccc4ccccc4c3-c3cccc4ccccc34)cc2)cc1 ; CC(C)(C)c1ccc(N(c2ccc3c(c2)-c2ccccc2C3(C)C)c2ccc3ccccc3c2-c2cccc3ccccc23)c2ccccc12]

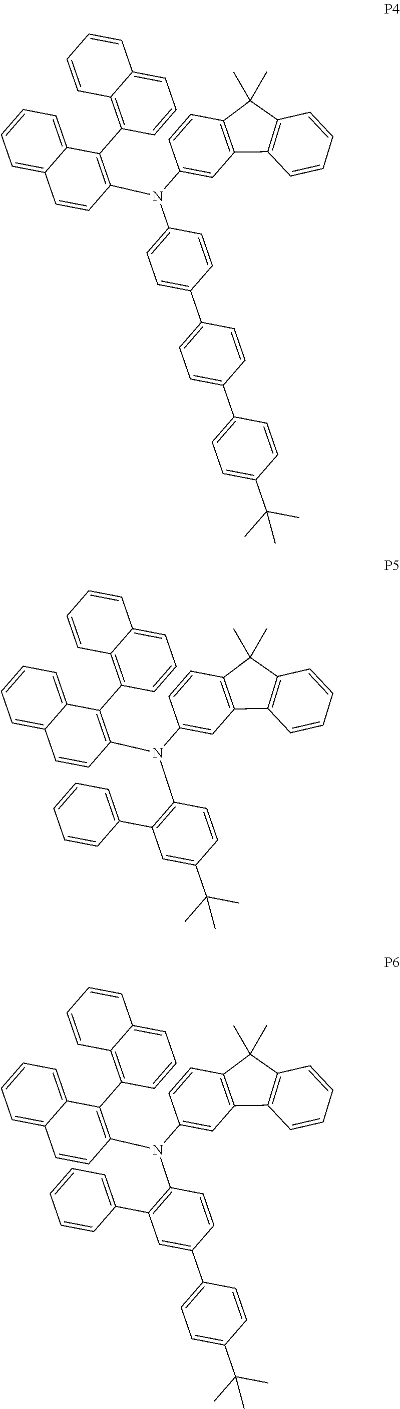 [SMILES: CC(C)(C)c1ccc(-c2ccc(-c3ccc(N(c4ccc5c(c4)-c4ccccc4C5(C)C)c4ccc5ccccc5c4-c4cccc5ccccc45)cc3)cc2)cc1 ; CC(C)(C)c1ccc(N(c2ccc3c(c2)-c2ccccc2C3(C)C)c2ccc3ccccc3c2-c2cccc3ccccc23)c(-c2ccccc2)c1 ; CC(C)(C)c1ccc(-c2ccc(N(c3ccc4c(c3)-c3ccccc3C4(C)C)c3ccc4ccccc4c3-c3cccc4ccccc34)c(-c3ccccc3)c2)cc1]

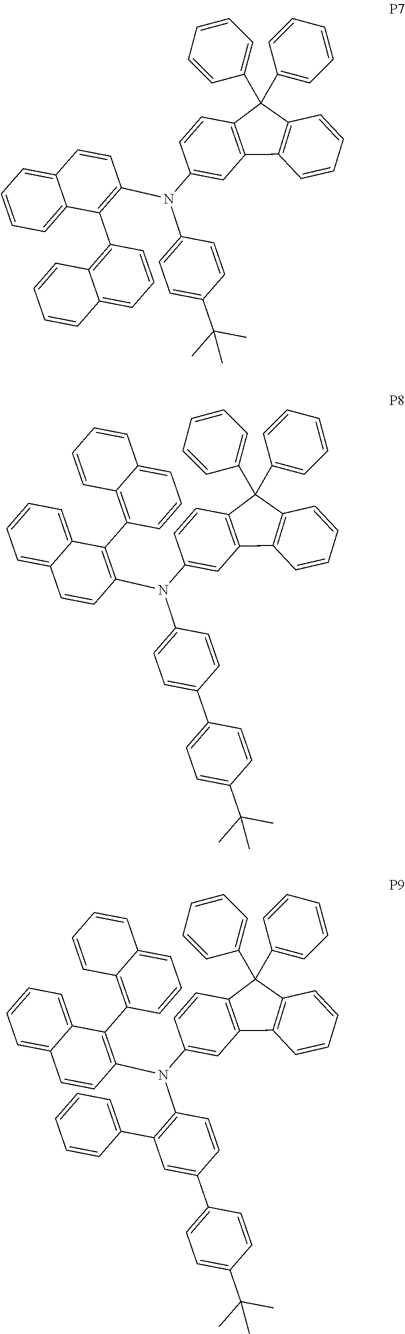 [SMILES: CC(C)(C)c1ccc(N(c2ccc3c(c2)-c2ccccc2C3(c2ccccc2)c2ccccc2)c2ccc3ccccc3c2-c2cccc3ccccc23)cc1 ; CC(C)(C)c1ccc(-c2ccc(N(c3ccc4c(c3)-c3ccccc3C4(c3ccccc3)c3ccccc3)c3ccc4ccccc4c3-c3cccc4ccccc34)cc2)cc1 ; CC(C)(C)c1ccc(-c2ccc(N(c3ccc4c(c3)-c3ccccc3C4(c3ccccc3)c3ccccc3)c3ccc4ccccc4c3-c3cccc4ccccc34)c(-c3ccccc3)c2)cc1]

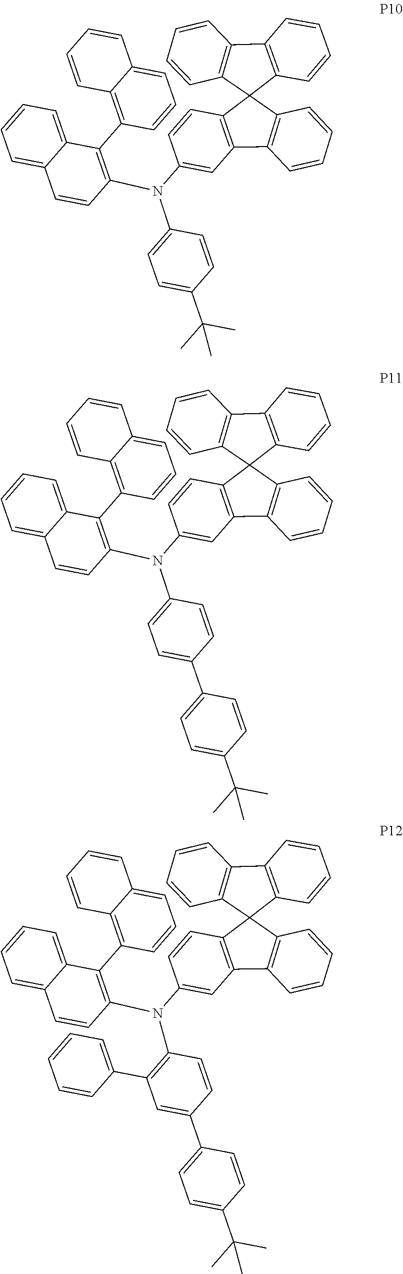

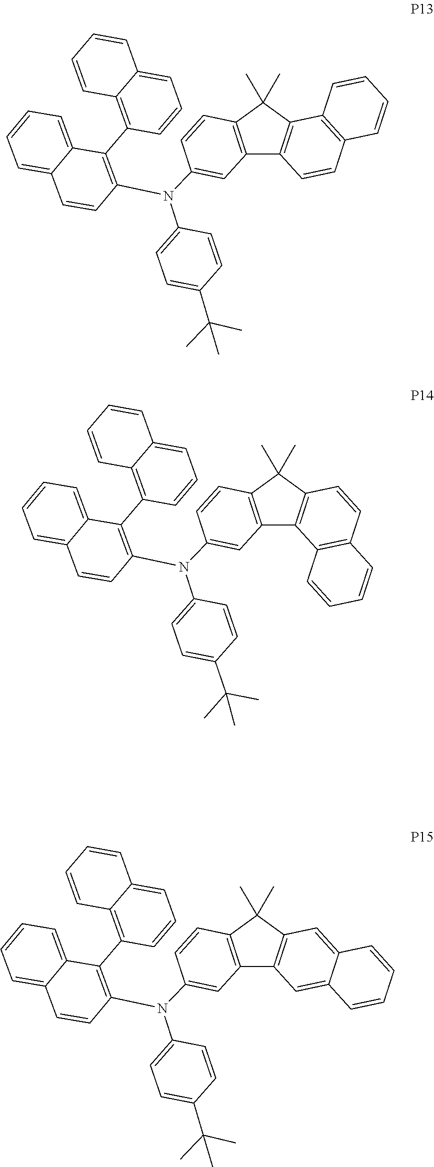 [SMILES: CC(C)(C)c1ccc(N(c2ccc3c(c2)-c2ccc4ccccc4c2C3(C)C)c2ccc3ccccc3c2-c2cccc3ccccc23)cc1 ; CC(C)(C)c1ccc(N(c2ccc3c(c2)-c2c(ccc4ccccc24)C3(C)C)c2ccc3ccccc3c2-c2cccc3ccccc23)cc1 ; CC(C)(C)c1ccc(N(c2ccc3c(c2)-c2cc4ccccc4cc2C3(C)C)c2ccc3ccccc3c2-c2cccc3ccccc23)cc1]

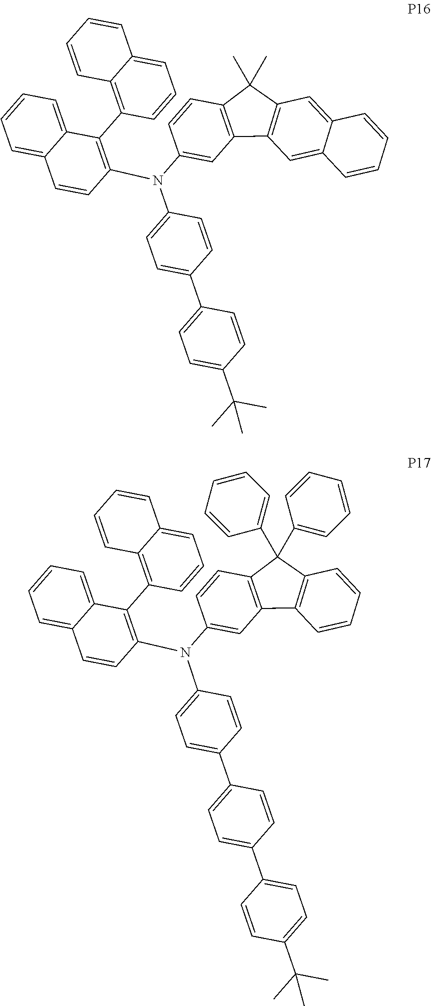

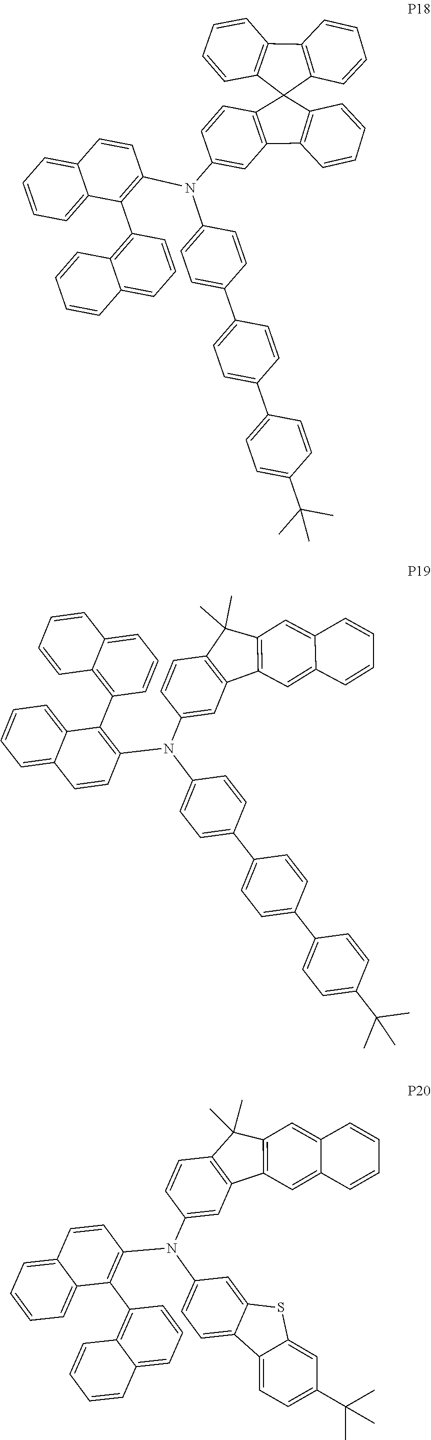 [SMILES: CC(C)(C)c1ccc(-c2ccc(-c3ccc(N(c4ccc5c(c4)-c4ccccc4C54c5ccccc5-c5ccccc54)c4ccc5ccccc5c4-c4cccc5ccccc45)cc3)cc2)cc1 ; CC(C)(C)c1ccc(-c2ccc(-c3ccc(N(c4ccc5c(c4)-c4cc6ccccc6cc4C5(C)C)c4ccc5ccccc5c4-c4cccc5ccccc45)cc3)cc2)cc1 ; CC(C)(C)c1ccc2c(c1)sc1cc(N(c3ccc4c(c3)-c3cc5ccccc5cc3C4(C)C)c3ccc4ccccc4c3-c3cccc4ccccc34)ccc12]

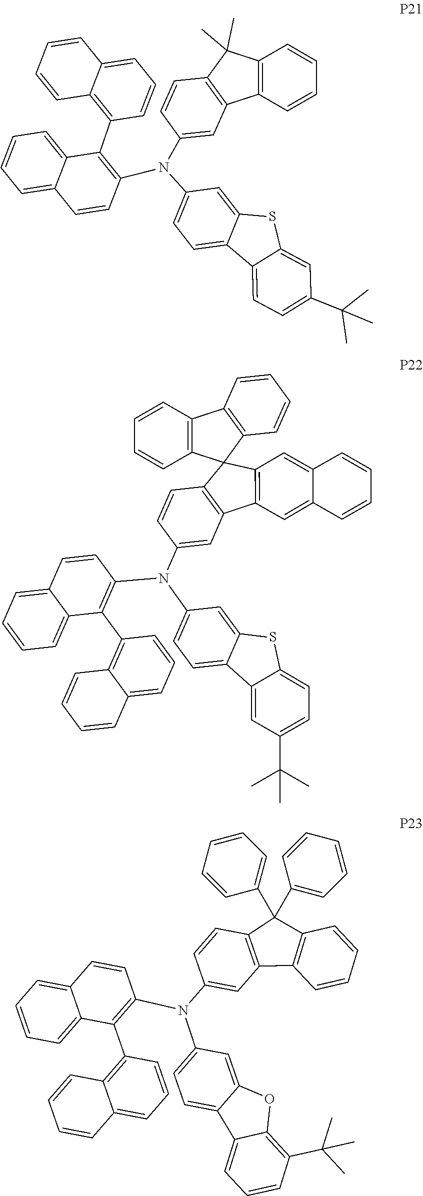

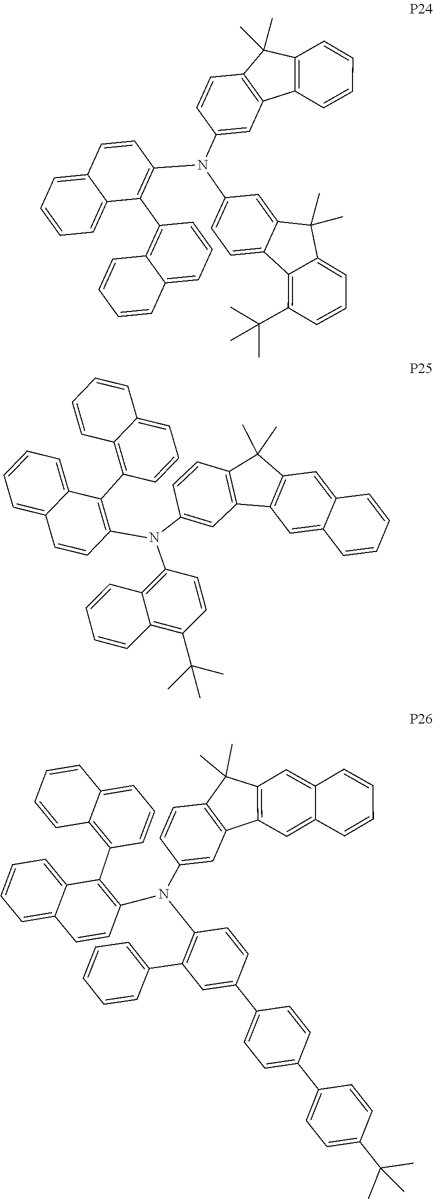 [SMILES: CC(C)(C)c1cccc2c1-c1ccc(N(c3ccc4c(c3)-c3ccccc3C4(C)C)c3ccc4ccccc4c3-c3cccc4ccccc34)cc1C2(C)C ; CC(C)(C)c1ccc(N(c2ccc3c(c2)-c2cc4ccccc4cc2C3(C)C)c2ccc3ccccc3c2-c2cccc3ccccc23)c2ccccc12 ; CC(C)(C)c1ccc(-c2ccc(-c3ccc(N(c4ccc5c(c4)-c4cc6ccccc6cc4C5(C)C)c4ccc5ccccc5c4-c4cccc5ccccc45)c(-c4ccccc4)c3)cc2)cc1]

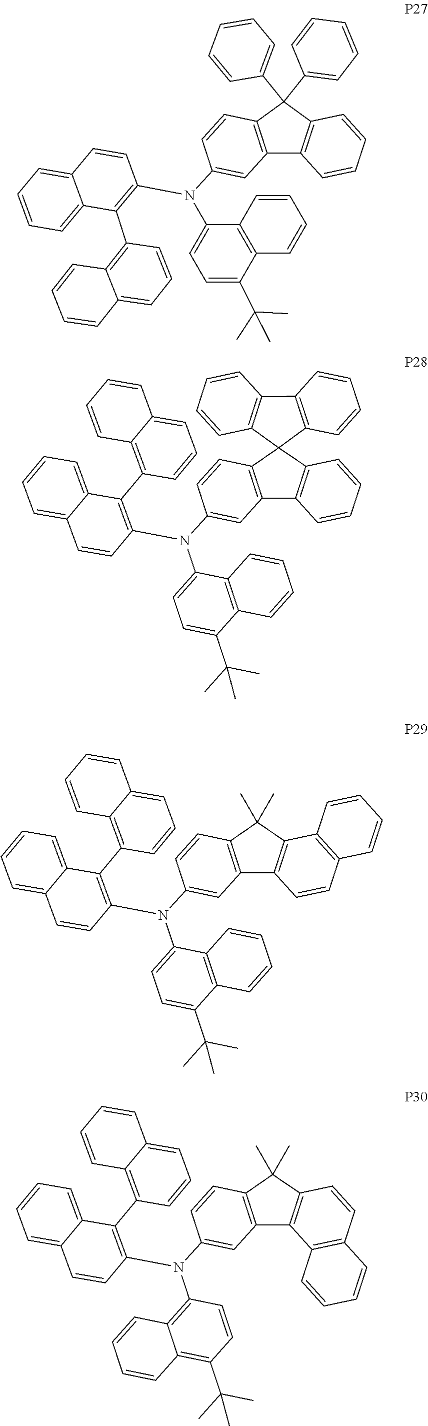

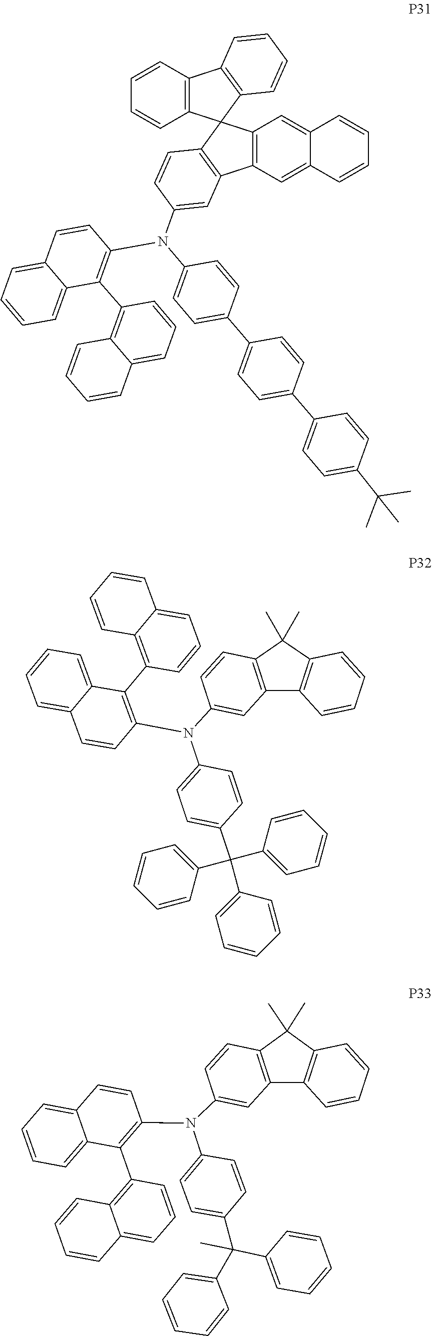 [SMILES: CC(C)(C)c1ccc(-c2ccc(-c3ccc(N(c4ccc5c(c4)-c4cc6ccccc6cc4C54c5ccccc5-c5ccccc54)c4ccc5ccccc5c4-c4cccc5ccccc45)cc3)cc2)cc1 ; CC1(C)c2ccccc2-c2cc(N(c3ccc(C(c4ccccc4)(c4ccccc4)c4ccccc4)cc3)c3ccc4ccccc4c3-c3cccc4ccccc34)ccc21 ; CC1(C)c2ccccc2-c2cc(N(c3ccc(C(C)(c4ccccc4)c4ccccc4)cc3)c3ccc4ccccc4c3-c3cccc4ccccc34)ccc21]

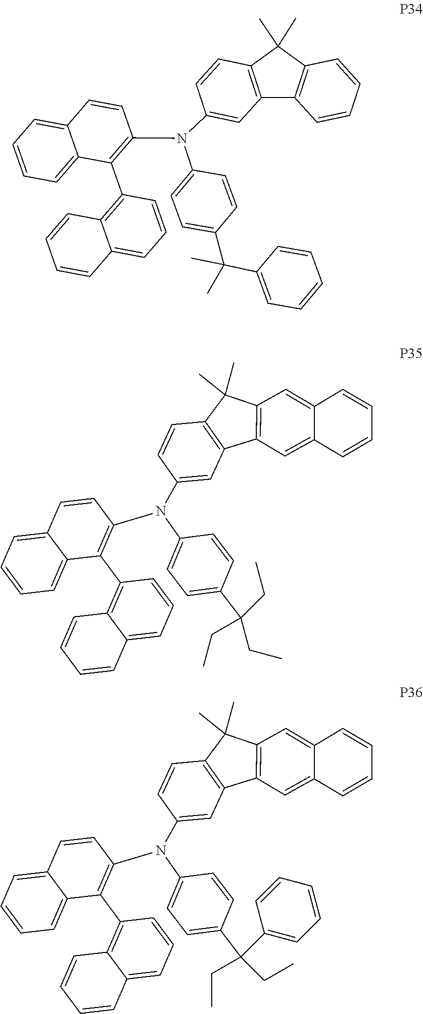

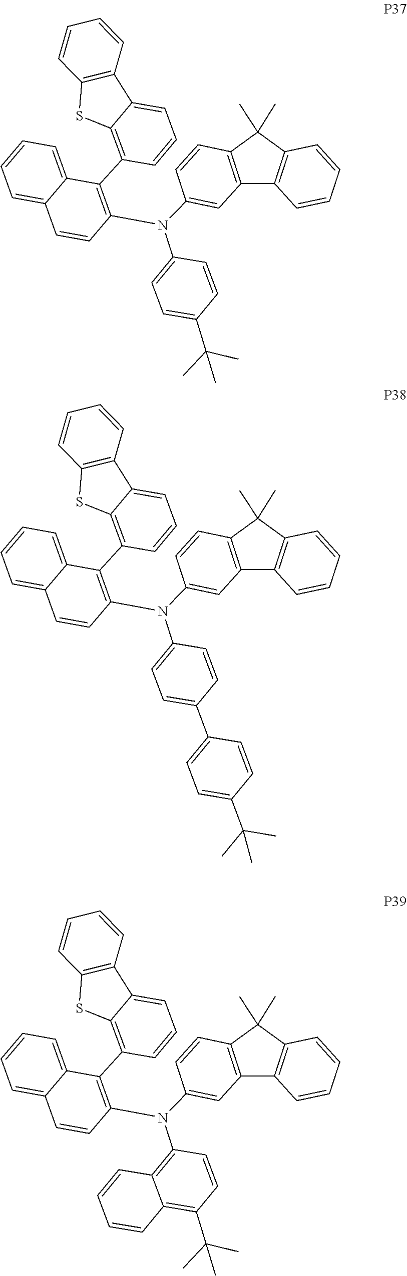 [SMILES: CC(C)(C)c1ccc(N(c2ccc3c(c2)-c2ccccc2C3(C)C)c2ccc3ccccc3c2-c2cccc3c2sc2ccccc23)cc1 ; CC(C)(C)c1ccc(-c2ccc(N(c3ccc4c(c3)-c3ccccc3C4(C)C)c3ccc4ccccc4c3-c3cccc4c3sc3ccccc34)cc2)cc1 ; CC(C)(C)c1ccc(N(c2ccc3c(c2)-c2ccccc2C3(C)C)c2ccc3ccccc3c2-c2cccc3c2sc2ccccc23)c2ccccc12]

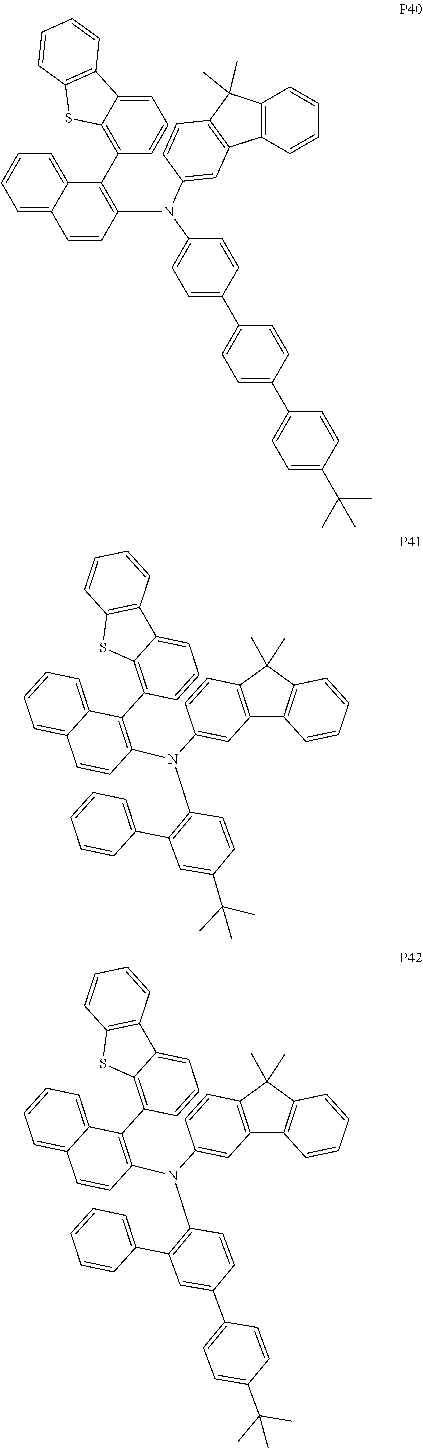 [SMILES: CC(C)(C)c1ccc(-c2ccc(-c3ccc(N(c4ccc5c(c4)-c4ccccc4C5(C)C)c4ccc5ccccc5c4-c4cccc5c4sc4ccccc45)cc3)cc2)cc1 ; CC(C)(C)c1ccc(N(c2ccc3c(c2)-c2ccccc2C3(C)C)c2ccc3ccccc3c2-c2cccc3c2sc2ccccc23)c(-c2ccccc2)c1 ; CC(C)(C)c1ccc(-c2ccc(N(c3ccc4c(c3)-c3ccccc3C4(C)C)c3ccc4ccccc4c3-c3cccc4c3sc3ccccc34)c(-c3ccccc3)c2)cc1]

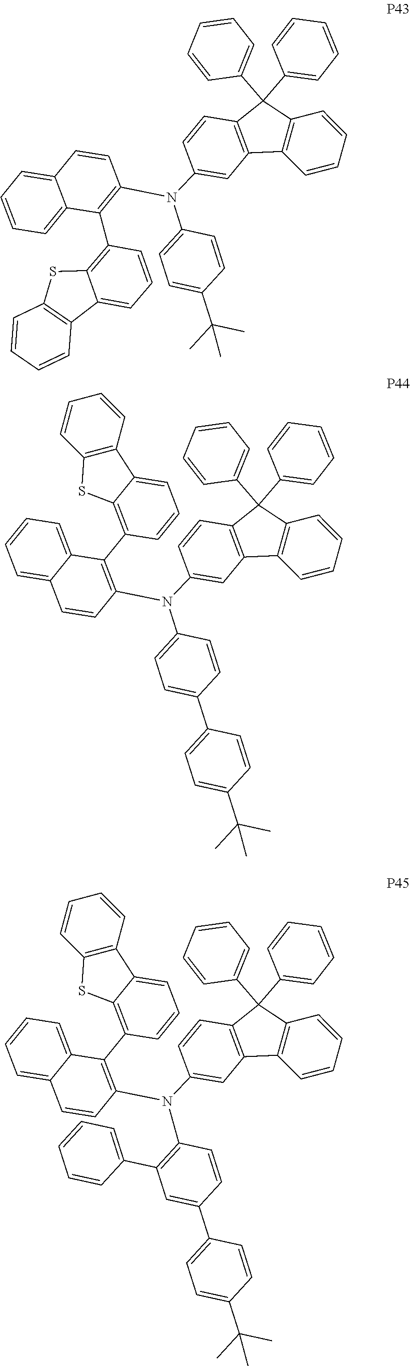

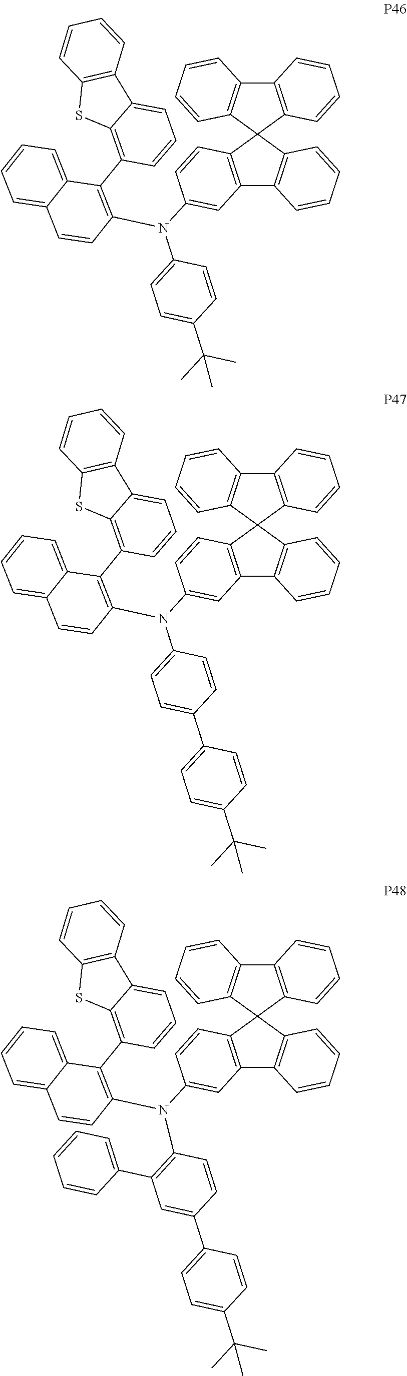 [SMILES: CC(C)(C)c1ccc(N(c2ccc3c(c2)-c2ccccc2C32c3ccccc3-c3ccccc32)c2ccc3ccccc3c2-c2cccc3c2sc2ccccc23)cc1 ; CC(C)(C)c1ccc(-c2ccc(N(c3ccc4c(c3)-c3ccccc3C43c4ccccc4-c4ccccc43)c3ccc4ccccc4c3-c3cccc4c3sc3ccccc34)cc2)cc1 ; CC(C)(C)c1ccc(-c2ccc(N(c3ccc4c(c3)-c3ccccc3C43c4ccccc4-c4ccccc43)c3ccc4ccccc4c3-c3cccc4c3sc3ccccc34)c(-c3ccccc3)c2)cc1]

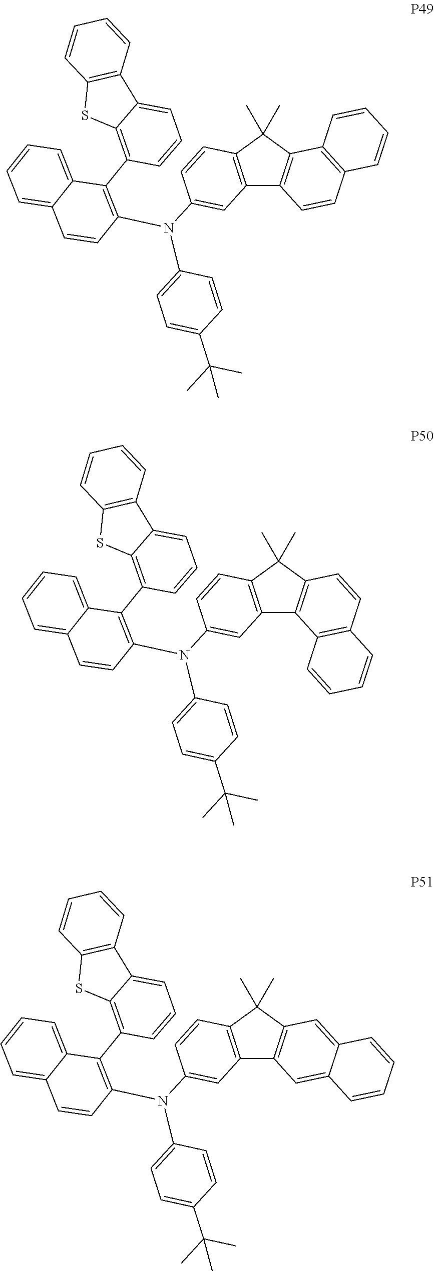 [SMILES: CC(C)(C)c1ccc(N(c2ccc3c(c2)-c2ccc4ccccc4c2C3(C)C)c2ccc3ccccc3c2-c2cccc3c2sc2ccccc23)cc1 ; CC(C)(C)c1ccc(N(c2ccc3c(c2)-c2c(ccc4ccccc24)C3(C)C)c2ccc3ccccc3c2-c2cccc3c2sc2ccccc23)cc1 ; CC(C)(C)c1ccc(N(c2ccc3c(c2)-c2cc4ccccc4cc2C3(C)C)c2ccc3ccccc3c2-c2cccc3c2sc2ccccc23)cc1]

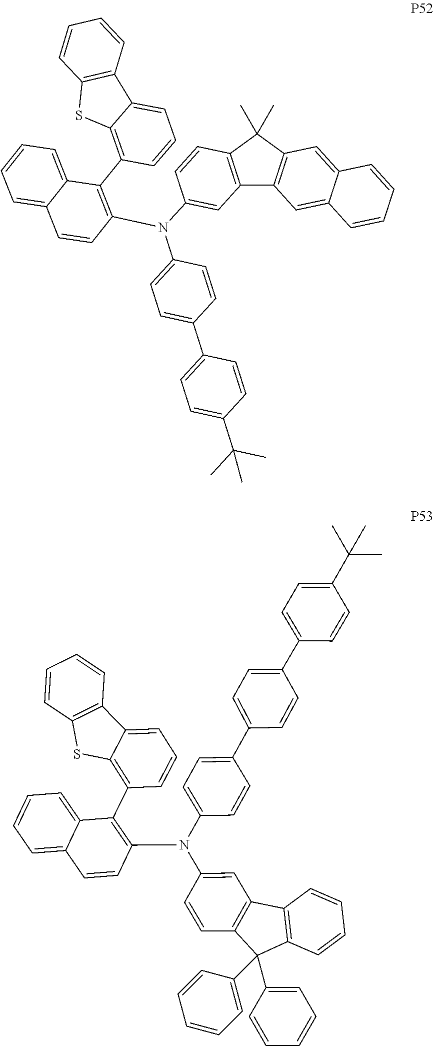 [SMILES: CC(C)(C)c1ccc(-c2ccc(N(c3ccc4c(c3)-c3cc5ccccc5cc3C4(C)C)c3ccc4ccccc4c3-c3cccc4c3sc3ccccc34)cc2)cc1 ; CC(C)(C)c1ccc(-c2ccc(-c3ccc(N(c4ccc5c(c4)-c4ccccc4C5(c4ccccc4)c4ccccc4)c4ccc5ccccc5c4-c4cccc5c4sc4ccccc45)cc3)cc2)cc1]

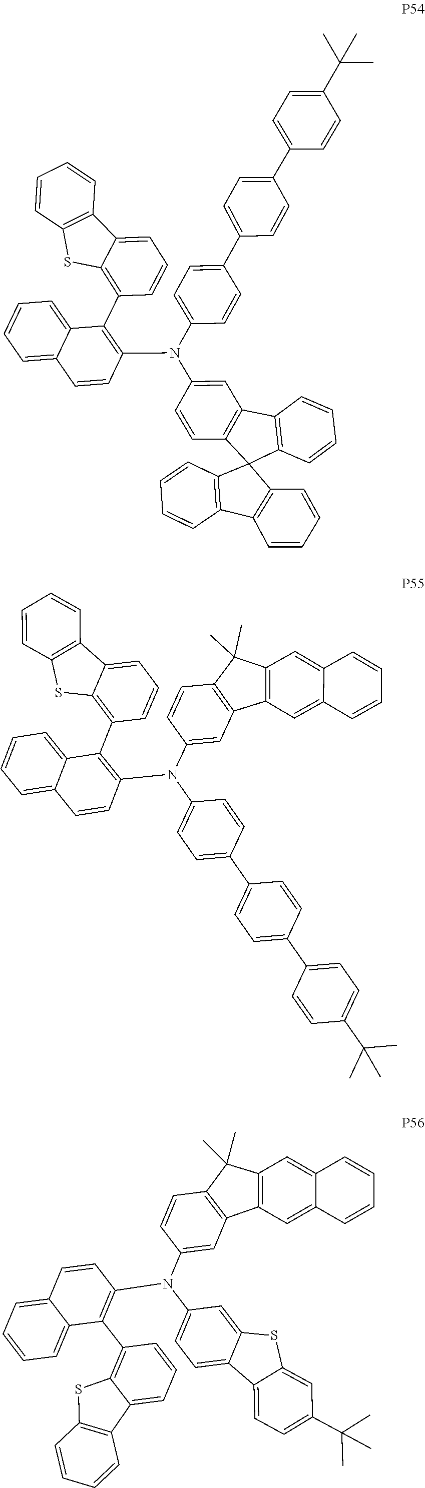 [SMILES: CC(C)(C)c1ccc(-c2ccc(-c3ccc(N(c4ccc5c(c4)-c4ccccc4C54c5ccccc5-c5ccccc54)c4ccc5ccccc5c4-c4cccc5c4sc4ccccc45)cc3)cc2)cc1 ; CC(C)(C)c1ccc(-c2ccc(-c3ccc(N(c4ccc5c(c4)-c4cc6ccccc6cc4C5(C)C)c4ccc5ccccc5c4-c4cccc5c4sc4ccccc45)cc3)cc2)cc1 ; CC(C)(C)c1ccc2c(c1)sc1cc(N(c3ccc4c(c3)-c3cc5ccccc5cc3C4(C)C)c3ccc4ccccc4c3-c3cccc4c3sc3ccccc34)ccc12]

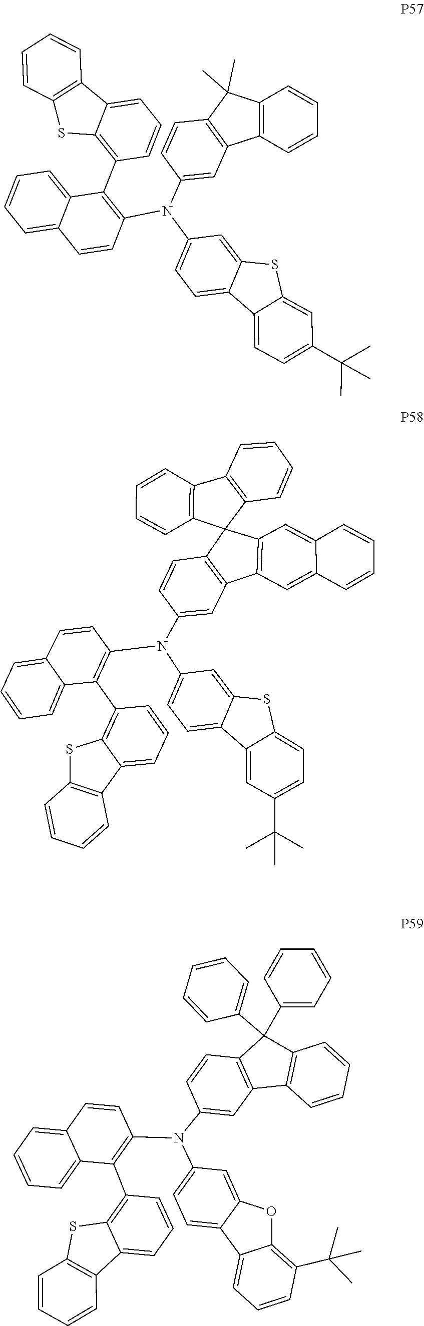 [SMILES: CC(C)(C)c1ccc2c(c1)sc1cc(N(c3ccc4c(c3)-c3ccccc3C4(C)C)c3ccc4ccccc4c3-c3cccc4c3sc3ccccc34)ccc12 ; CC(C)(C)c1ccc2sc3cc(N(c4ccc5c(c4)-c4cc6ccccc6cc4C54c5ccccc5-c5ccccc54)c4ccc5ccccc5c4-c4cccc5c4sc4ccccc45)ccc3c2c1 ; CC(C)(C)c1cccc2c1oc1cc(N(c3ccc4c(c3)-c3ccccc3C4(c3ccccc3)c3ccccc3)c3ccc4ccccc4c3-c3cccc4c3sc3ccccc34)ccc12]

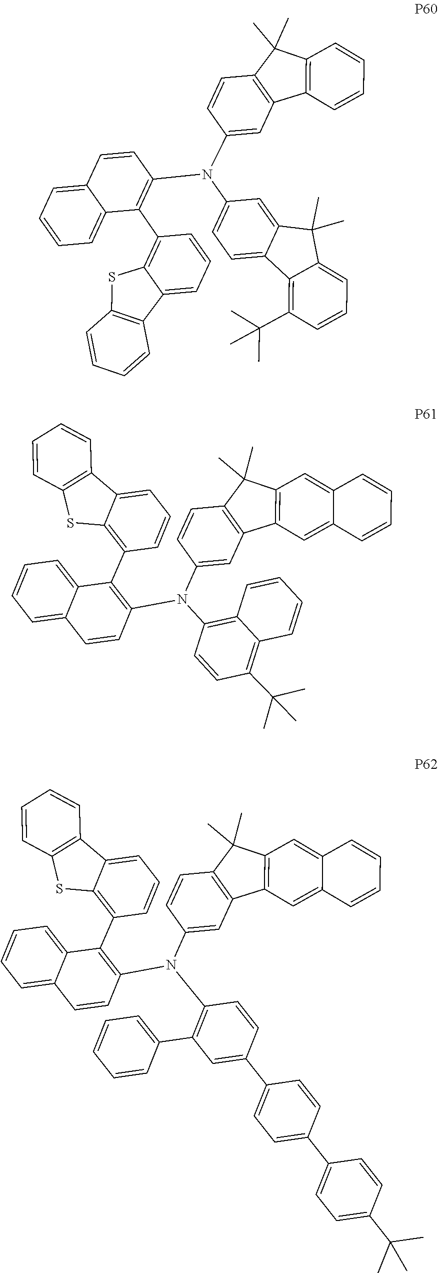 [SMILES: CC(C)(C)c1cccc2c1-c1ccc(N(c3ccc4c(c3)-c3ccccc3C4(C)C)c3ccc4ccccc4c3-c3cccc4c3sc3ccccc34)cc1C2(C)C ; CC(C)(C)c1ccc(N(c2ccc3c(c2)-c2cc4ccccc4cc2C3(C)C)c2ccc3ccccc3c2-c2cccc3c2sc2ccccc23)c2ccccc12 ; CC(C)(C)c1ccc(-c2ccc(-c3ccc(N(c4ccc5c(c4)-c4cc6ccccc6cc4C5(C)C)c4ccc5ccccc5c4-c4cccc5c4sc4ccccc45)c(-c4ccccc4)c3)cc2)cc1]

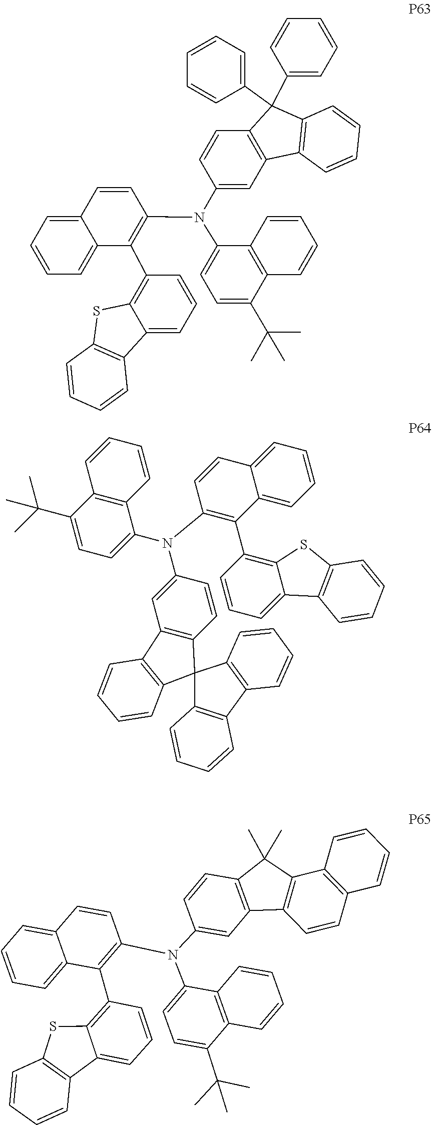

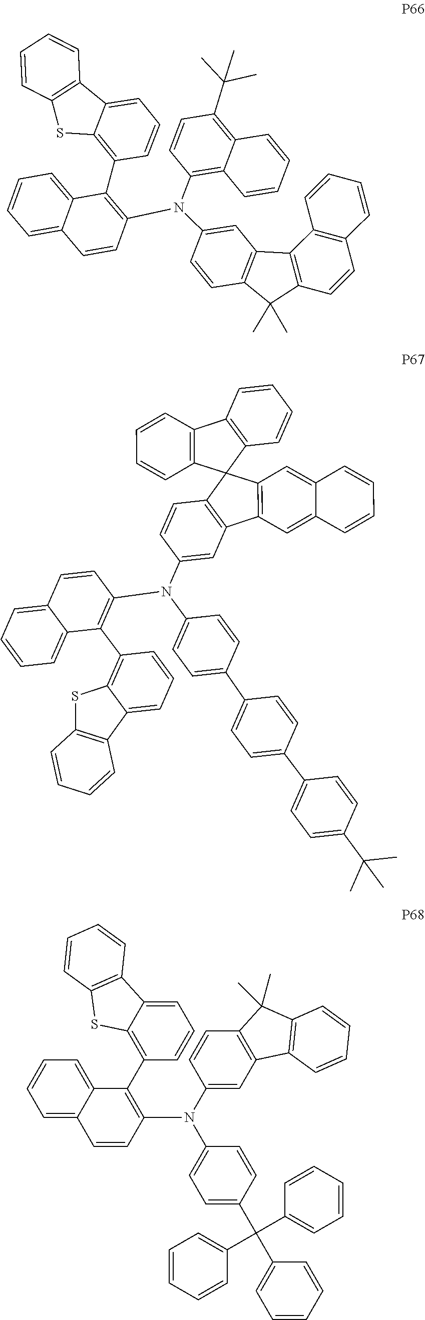 [SMILES: CC(C)(C)c1ccc(N(c2ccc3c(c2)-c2c(ccc4ccccc24)C3(C)C)c2ccc3ccccc3c2-c2cccc3c2sc2ccccc23)c2ccccc12 ; CC(C)(C)c1ccc(-c2ccc(-c3ccc(N(c4ccc5c(c4)-c4cc6ccccc6cc4C54c5ccccc5-c5ccccc54)c4ccc5ccccc5c4-c4cccc5c4sc4ccccc45)cc3)cc2)cc1 ; CC1(C)c2ccccc2-c2cc(N(c3ccc(C(c4ccccc4)(c4ccccc4)c4ccccc4)cc3)c3ccc4ccccc4c3-c3cccc4c3sc3ccccc34)ccc21]

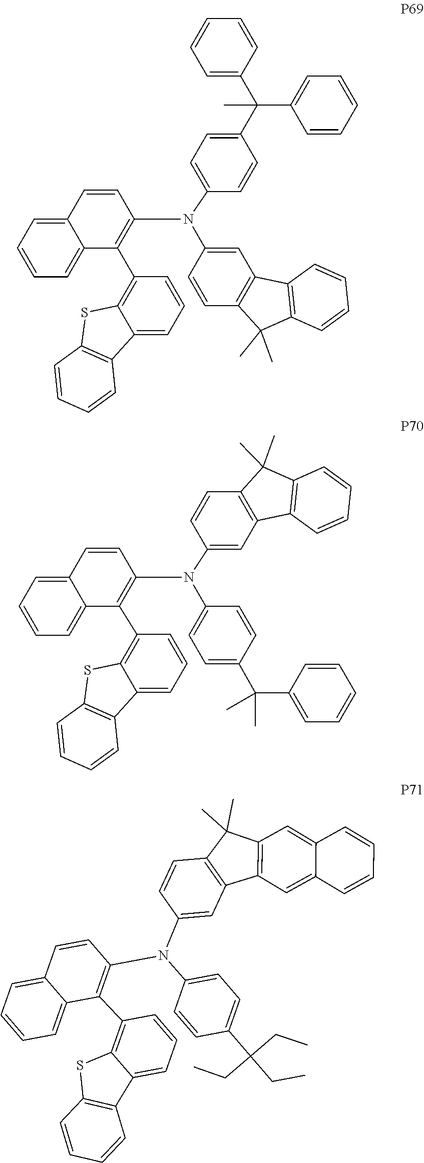 [SMILES: CC1(C)c2ccccc2-c2cc(N(c3ccc(C(C)(c4ccccc4)c4ccccc4)cc3)c3ccc4ccccc4c3-c3cccc4c3sc3ccccc34)ccc21 ; CC(C)(c1ccccc1)c1ccc(N(c2ccc3c(c2)-c2ccccc2C3(C)C)c2ccc3ccccc3c2-c2cccc3c2sc2ccccc23)cc1 ; CCC(CC)(CC)c1ccc(N(c2ccc3c(c2)-c2cc4ccccc4cc2C3(C)C)c2ccc3ccccc3c2-c2cccc3c2sc2ccccc23)cc1]

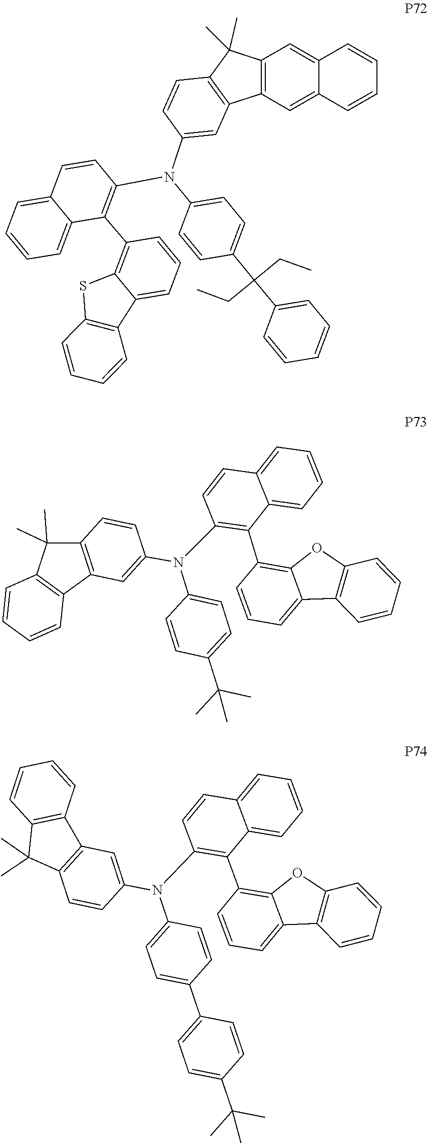 [SMILES: CCC(CC)(c1ccccc1)c1ccc(N(c2ccc3c(c2)-c2cc4ccccc4cc2C3(C)C)c2ccc3ccccc3c2-c2cccc3c2sc2ccccc23)cc1 ; CC(C)(C)c1ccc(N(c2ccc3c(c2)-c2ccccc2C3(C)C)c2ccc3ccccc3c2-c2cccc3c2oc2ccccc23)cc1 ; CC(C)(C)c1ccc(-c2ccc(N(c3ccc4c(c3)-c3ccccc3C4(C)C)c3ccc4ccccc4c3-c3cccc4c3oc3ccccc34)cc2)cc1]

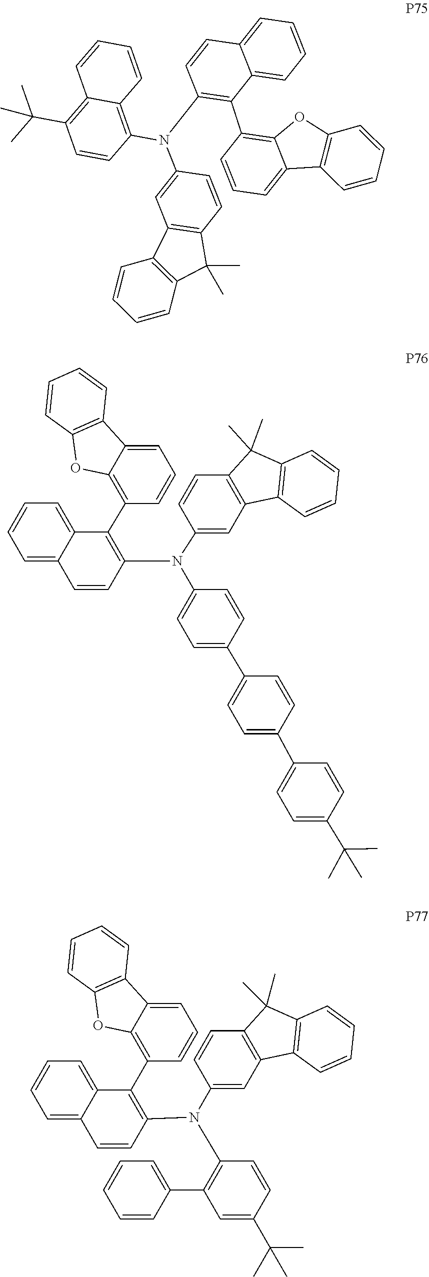 [SMILES: CC(C)(C)c1ccc(N(c2ccc3c(c2)-c2ccccc2C3(C)C)c2ccc3ccccc3c2-c2cccc3c2oc2ccccc23)c2ccccc12 ; CC(C)(C)c1ccc(-c2ccc(-c3ccc(N(c4ccc5c(c4)-c4ccccc4C5(C)C)c4ccc5ccccc5c4-c4cccc5c4oc4ccccc45)cc3)cc2)cc1 ; CC(C)(C)c1ccc(N(c2ccc3c(c2)-c2ccccc2C3(C)C)c2ccc3ccccc3c2-c2cccc3c2oc2ccccc23)c(-c2ccccc2)c1]

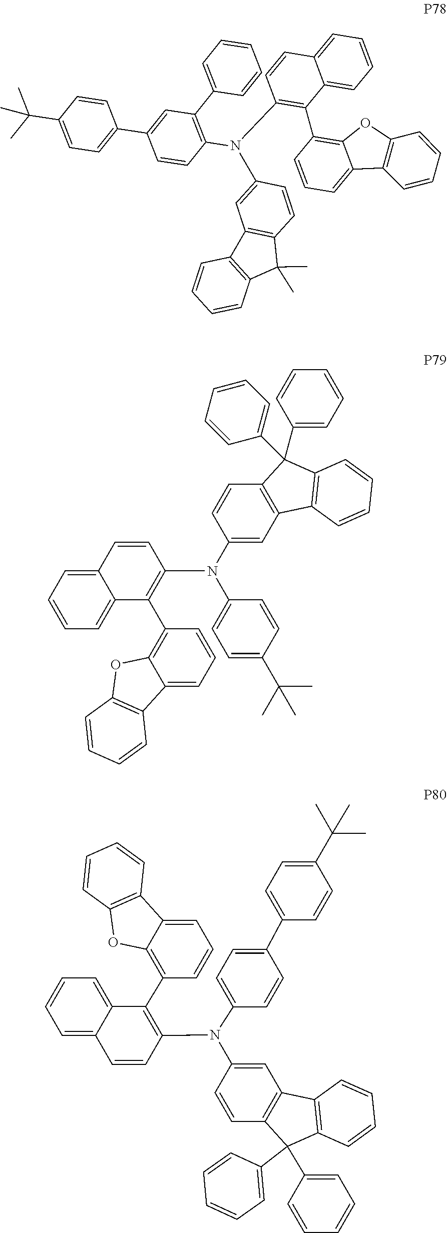 [SMILES: CC(C)(C)c1ccc(-c2ccc(N(c3ccc4c(c3)-c3ccccc3C4(C)C)c3ccc4ccccc4c3-c3cccc4c3oc3ccccc34)c(-c3ccccc3)c2)cc1 ; CC(C)(C)c1ccc(N(c2ccc3c(c2)-c2ccccc2C3(c2ccccc2)c2ccccc2)c2ccc3ccccc3c2-c2cccc3c2oc2ccccc23)cc1 ; CC(C)(C)c1ccc(-c2ccc(N(c3ccc4c(c3)-c3ccccc3C4(c3ccccc3)c3ccccc3)c3ccc4ccccc4c3-c3cccc4c3oc3ccccc34)cc2)cc1]

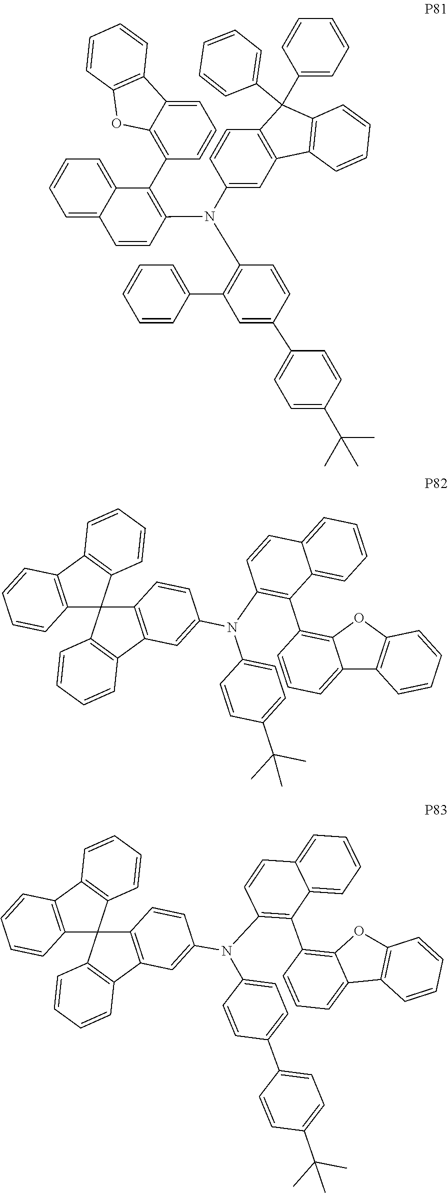 [SMILES: C=C/C(=C\C1=C(C)C(c2ccccc2)(c2ccccc2)c2ccccc21)N(c1ccc(-c2ccc(C(C)(C)C)cc2)cc1-c1ccccc1)c1ccc2ccccc2c1-c1cccc2c1oc1ccccc12 ; CC(C)(C)c1ccc(N(c2ccc3c(c2)-c2ccccc2C32c3ccccc3-c3ccccc32)c2ccc3ccccc3c2-c2cccc3c2oc2ccccc23)cc1 ; CC(C)(C)c1ccc(-c2ccc(N(c3ccc4c(c3)-c3ccccc3C43c4ccccc4-c4ccccc43)c3ccc4ccccc4c3-c3cccc4c3oc3ccccc34)cc2)cc1]

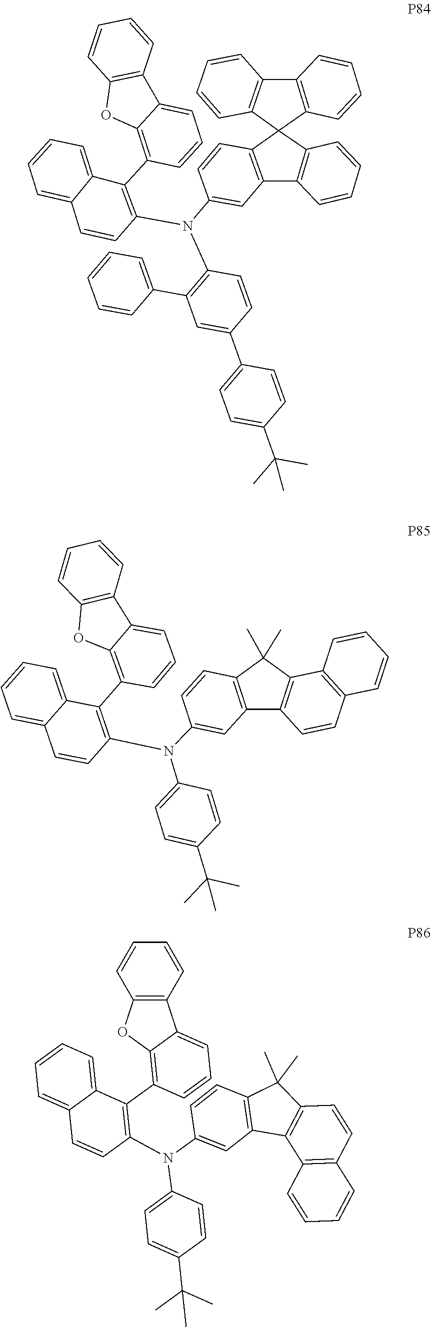

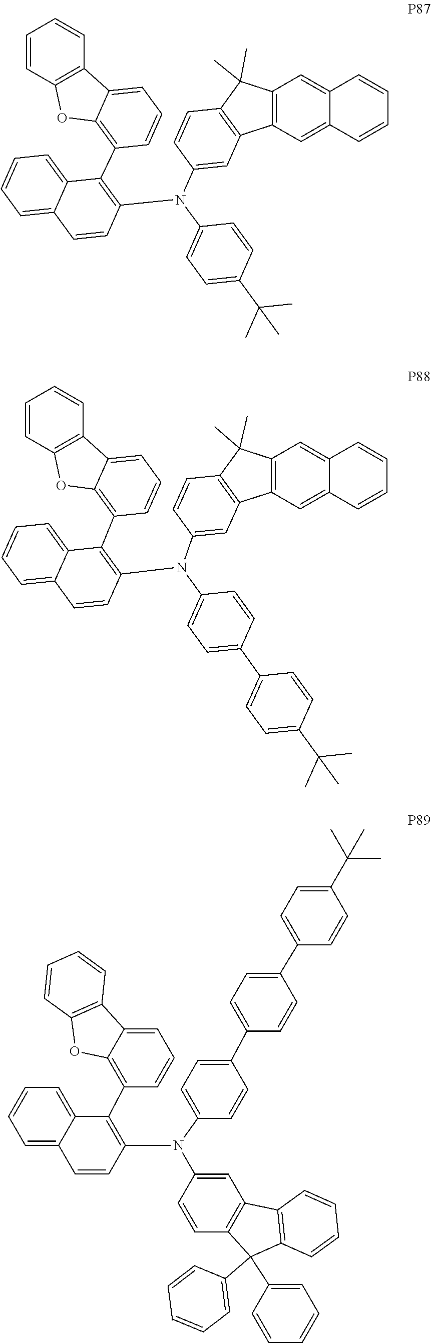

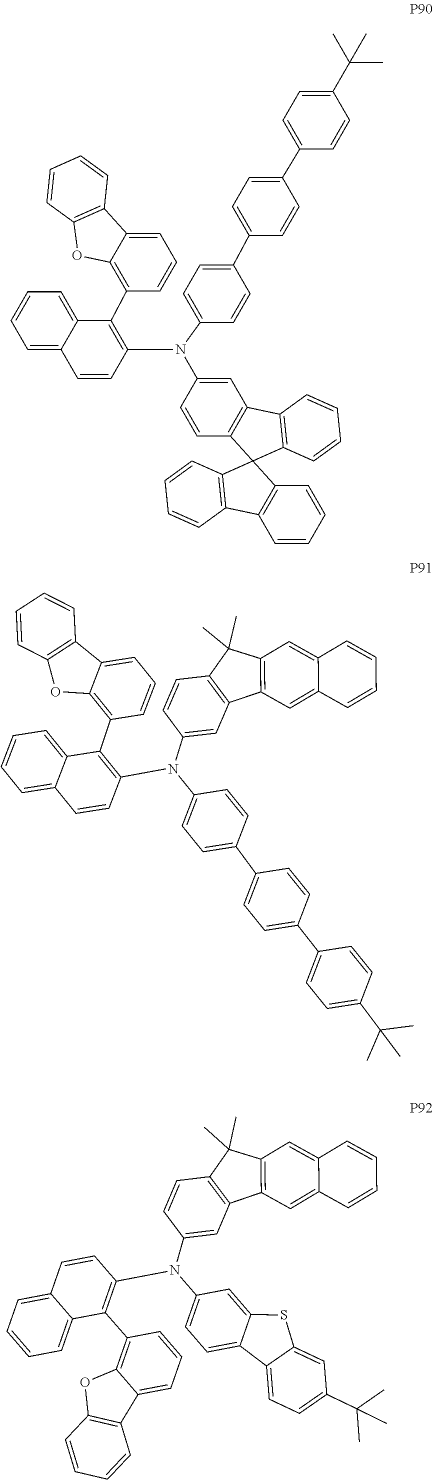 [SMILES: CC(C)(C)c1ccc(-c2ccc(-c3ccc(N(c4ccc5c(c4)-c4ccccc4C54c5ccccc5-c5ccccc54)c4ccc5ccccc5c4-c4cccc5c4oc4ccccc45)cc3)cc2)cc1 ; CC(C)(C)c1ccc(-c2ccc(-c3ccc(N(c4ccc5c(c4)-c4cc6ccccc6cc4C5(C)C)c4ccc5ccccc5c4-c4cccc5c4oc4ccccc45)cc3)cc2)cc1 ; CC(C)(C)c1ccc2c(c1)sc1cc(N(c3ccc4c(c3)-c3cc5ccccc5cc3C4(C)C)c3ccc4ccccc4c3-c3cccc4c3oc3ccccc34)ccc12]

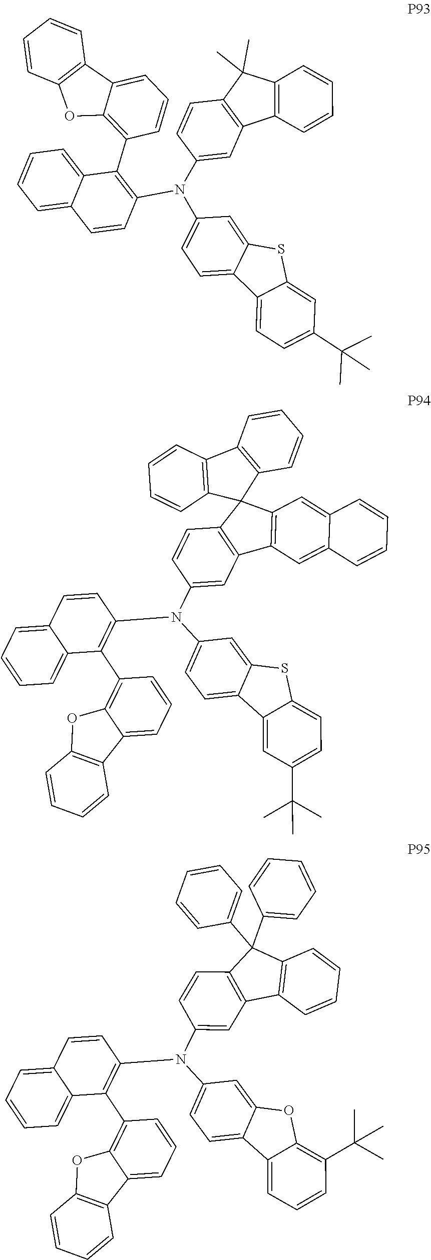

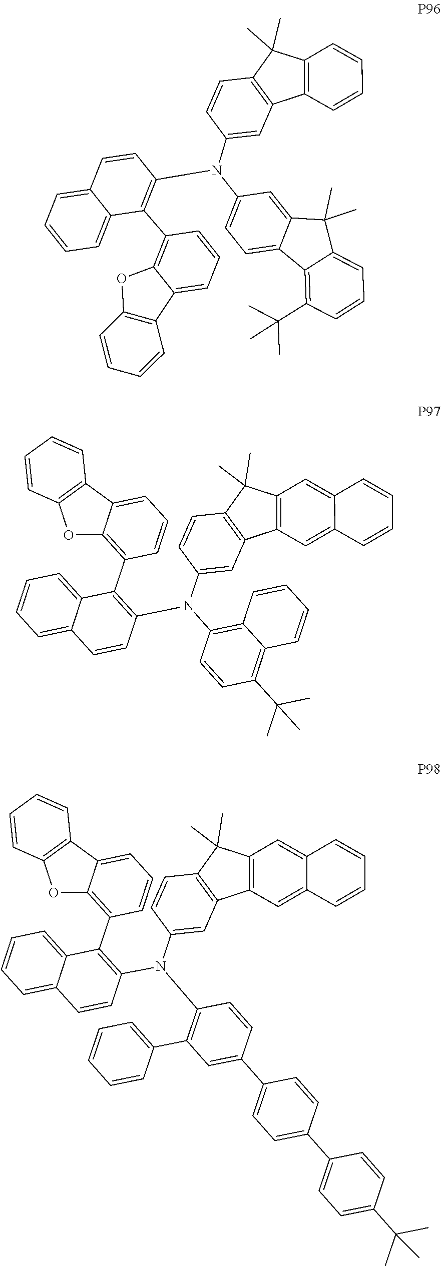 [SMILES: CC(C)(C)c1cccc2c1-c1ccc(N(c3ccc4c(c3)-c3ccccc3C4(C)C)c3ccc4ccccc4c3-c3cccc4c3oc3ccccc34)cc1C2(C)C ; CC(C)(C)c1ccc(N(c2ccc3c(c2)-c2cc4ccccc4cc2C3(C)C)c2ccc3ccccc3c2-c2cccc3c2oc2ccccc23)c2ccccc12 ; CC(C)(C)c1ccc(-c2ccc(-c3ccc(N(c4ccc5c(c4)-c4cc6ccccc6cc4C5(C)C)c4ccc5ccccc5c4-c4cccc5c4oc4ccccc45)c(-c4ccccc4)c3)cc2)cc1]

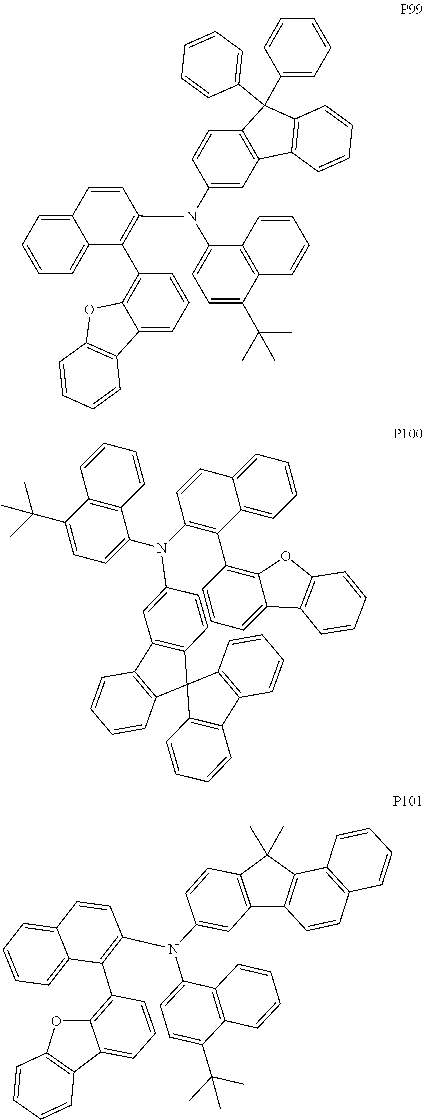

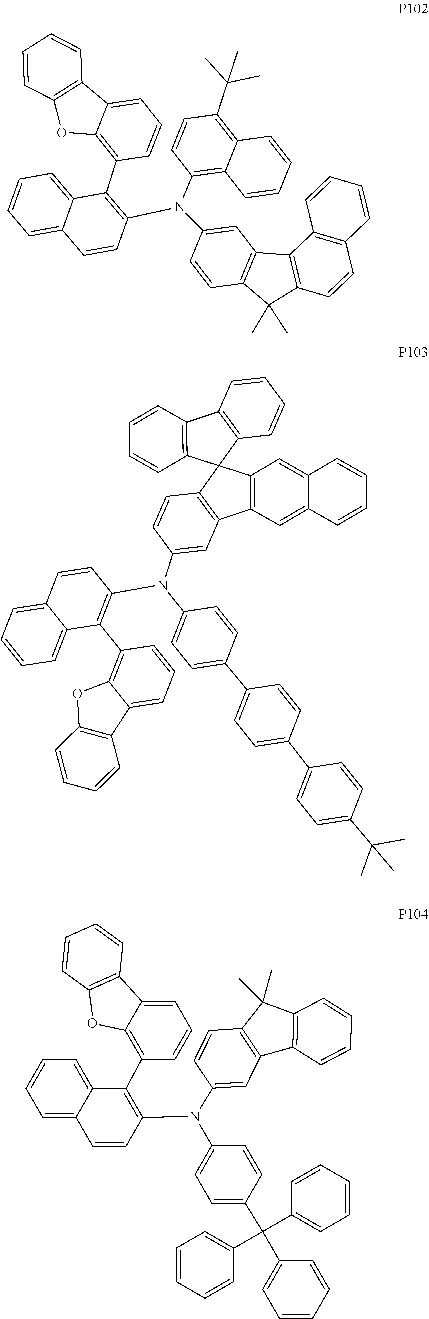

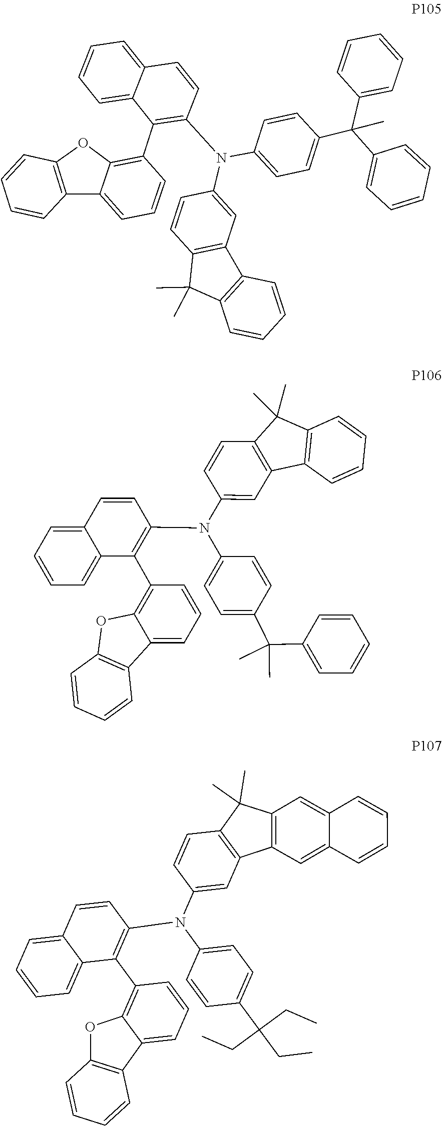 [SMILES: CC1(C)c2ccccc2-c2cc(N(c3ccc(C(C)(c4ccccc4)c4ccccc4)cc3)c3ccc4ccccc4c3-c3cccc4c3oc3ccccc34)ccc21 ; CC(C)(c1ccccc1)c1ccc(N(c2ccc3c(c2)-c2ccccc2C3(C)C)c2ccc3ccccc3c2-c2cccc3c2oc2ccccc23)cc1 ; CCC(CC)(CC)c1ccc(N(c2ccc3c(c2)-c2cc4ccccc4cc2C3(C)C)c2ccc3ccccc3c2-c2cccc3c2oc2ccccc23)cc1]

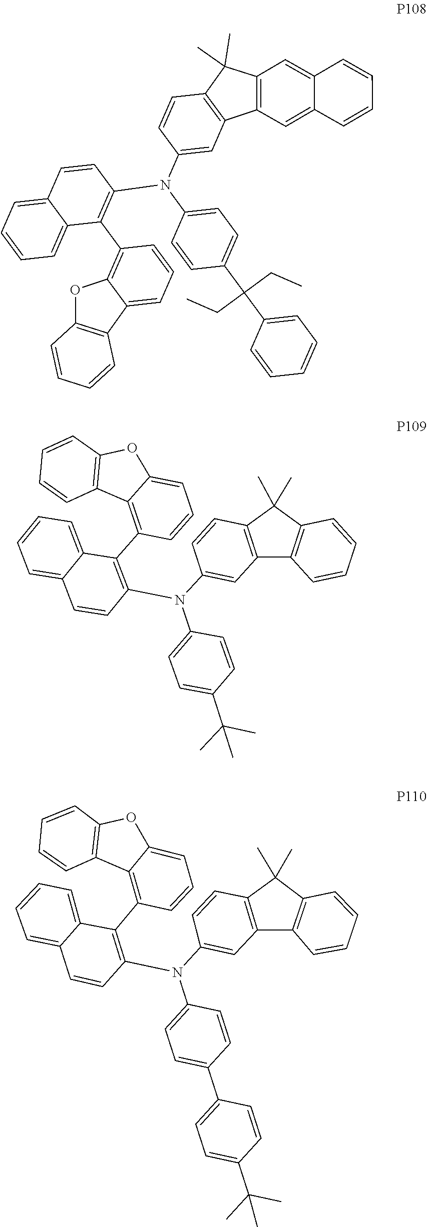 [SMILES: CCC(CC)(c1ccccc1)c1ccc(N(c2ccc3c(c2)-c2cc4ccccc4cc2C3(C)C)c2ccc3ccccc3c2-c2cccc3c2oc2ccccc23)cc1 ; CC(C)(C)c1ccc(N(c2ccc3c(c2)-c2ccccc2C3(C)C)c2ccc3ccccc3c2-c2cccc3oc4ccccc4c23)cc1 ; CC(C)(C)c1ccc(-c2ccc(N(c3ccc4c(c3)-c3ccccc3C4(C)C)c3ccc4ccccc4c3-c3cccc4oc5ccccc5c34)cc2)cc1]

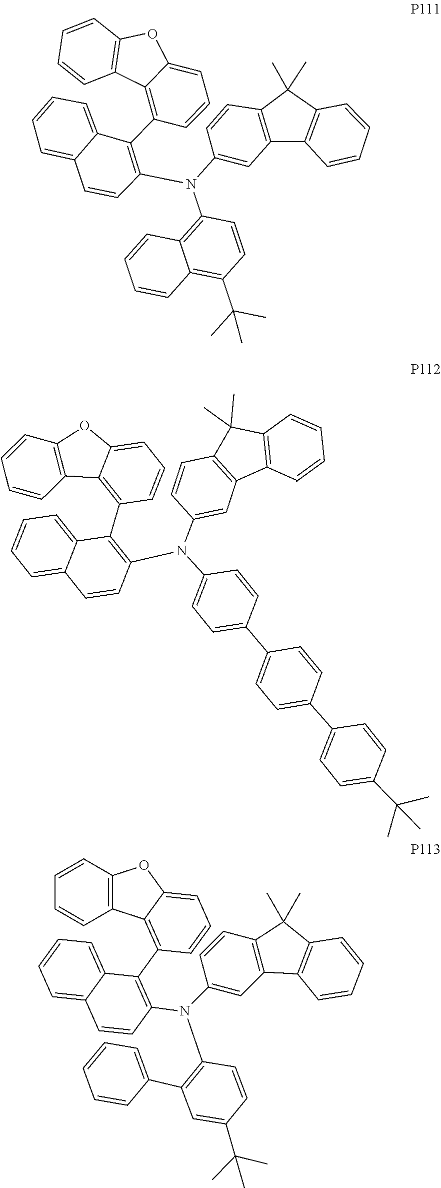 [SMILES: CC(C)(C)c1ccc(N(c2ccc3c(c2)-c2ccccc2C3(C)C)c2ccc3ccccc3c2-c2cccc3oc4ccccc4c23)c2ccccc12 ; Cc1cccc2c1-c1cc(N(c3ccc(-c4ccc(-c5ccc(C(C)(C)C)cc5)cc4)cc3)c3ccc4ccccc4c3-c3cccc4oc5ccccc5c34)ccc1C2(C)C ; CC(C)(C)c1ccc(N(c2ccc3c(c2)-c2ccccc2C3(C)C)c2ccc3ccccc3c2-c2cccc3oc4ccccc4c23)c(-c2ccccc2)c1]

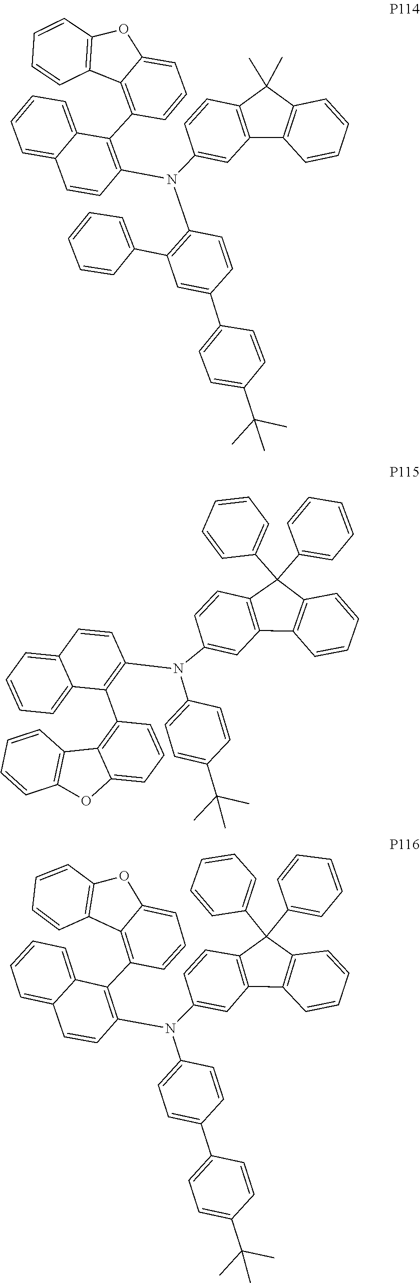

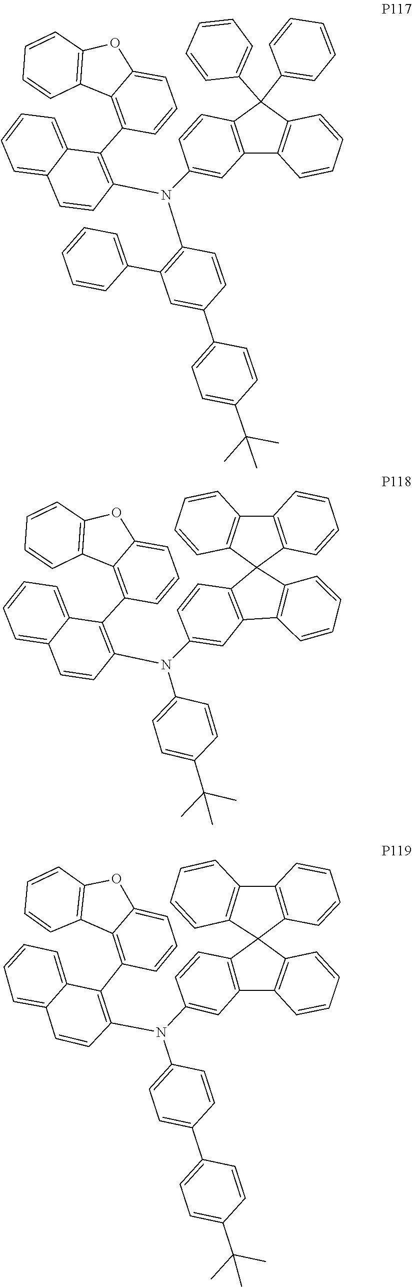

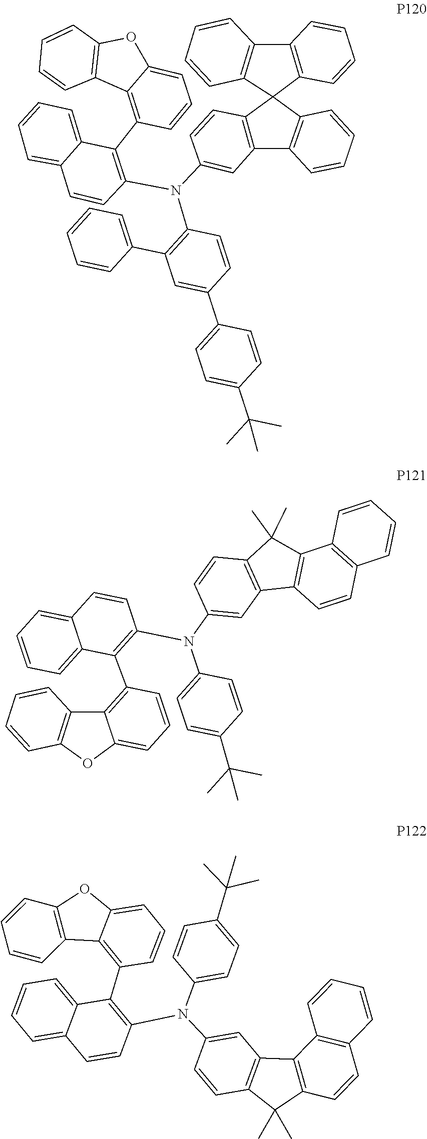 [SMILES: CC(C)(C)c1ccc(-c2ccc(N(c3ccc4c(c3)-c3ccccc3C43c4ccccc4-c4ccccc43)c3ccc4ccccc4c3-c3cccc4oc5ccccc5c34)c(-c3ccccc3)c2)cc1 ; CC(C)(C)c1ccc(N(c2ccc3c(c2)-c2ccc4ccccc4c2C3(C)C)c2ccc3ccccc3c2-c2cccc3oc4ccccc4c23)cc1 ; CC(C)(C)c1ccc(N(c2ccc3c(c2)-c2c(ccc4ccccc24)C3(C)C)c2ccc3ccccc3c2-c2cccc3oc4ccccc4c23)cc1]

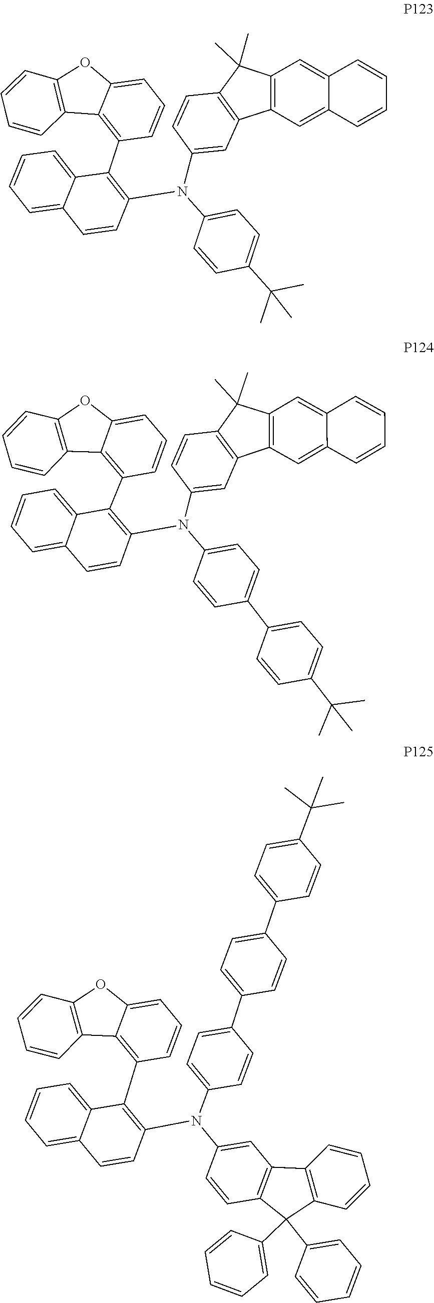

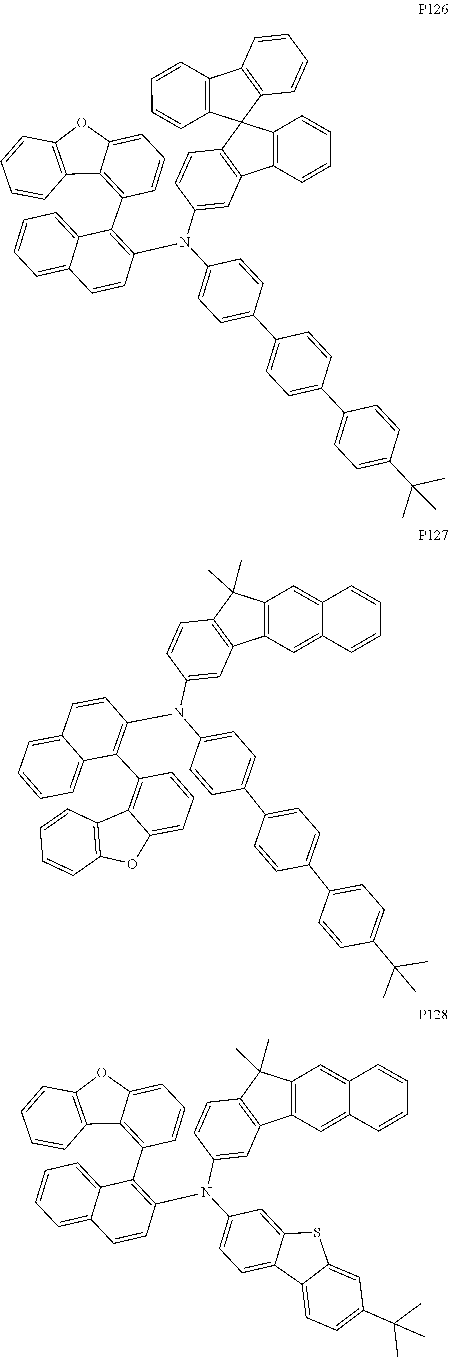 [SMILES: CC(C)(C)c1ccc(-c2ccc(-c3ccc(N(c4ccc5c(c4)-c4ccccc4C54c5ccccc5-c5ccccc54)c4ccc5ccccc5c4-c4cccc5oc6ccccc6c45)cc3)cc2)cc1 ; CC(C)(C)c1ccc(-c2ccc(-c3ccc(N(c4ccc5c(c4)-c4cc6ccccc6cc4C5(C)C)c4ccc5ccccc5c4-c4cccc5oc6ccccc6c45)cc3)cc2)cc1 ; CC(C)(C)c1ccc2c(c1)sc1cc(N(c3ccc4c(c3)-c3cc5ccccc5cc3C4(C)C)c3ccc4ccccc4c3-c3cccc4oc5ccccc5c34)ccc12]

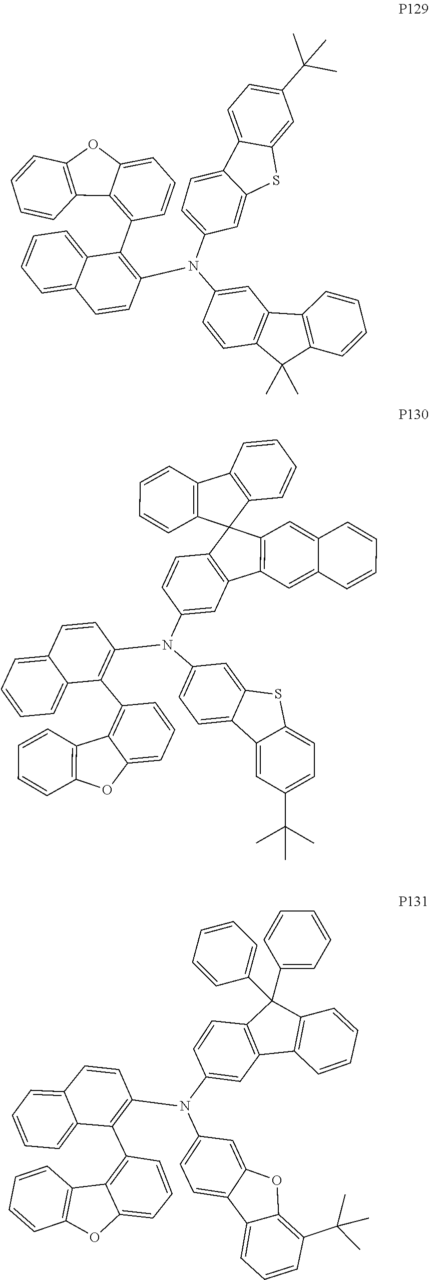 [SMILES: CC(C)(C)c1ccc2c(c1)sc1cc(N(c3ccc4c(c3)-c3ccccc3C4(C)C)c3ccc4ccccc4c3-c3cccc4oc5ccccc5c34)ccc12 ; CC(C)(C)c1ccc2sc3cc(N(c4ccc5c(c4)-c4cc6ccccc6cc4C54c5ccccc5-c5ccccc54)c4ccc5ccccc5c4-c4cccc5oc6ccccc6c45)ccc3c2c1 ; CC(C)(C)c1cccc2c1oc1cc(N(c3ccc4c(c3)-c3ccccc3C4(c3ccccc3)c3ccccc3)c3ccc4ccccc4c3-c3cccc4oc5ccccc5c34)ccc12]

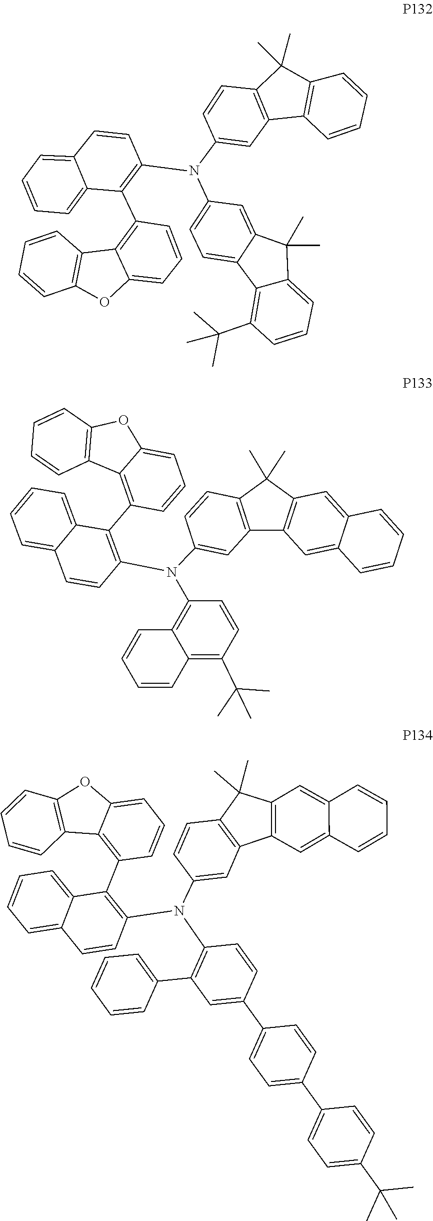

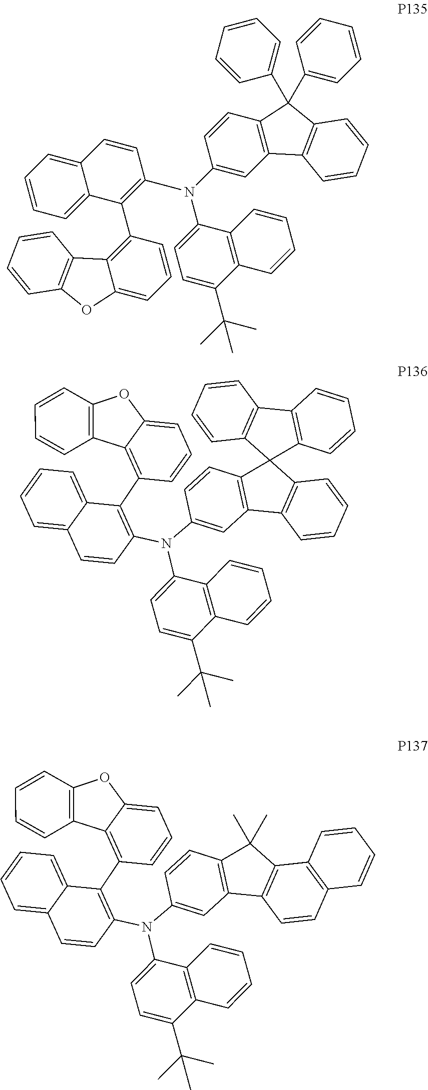

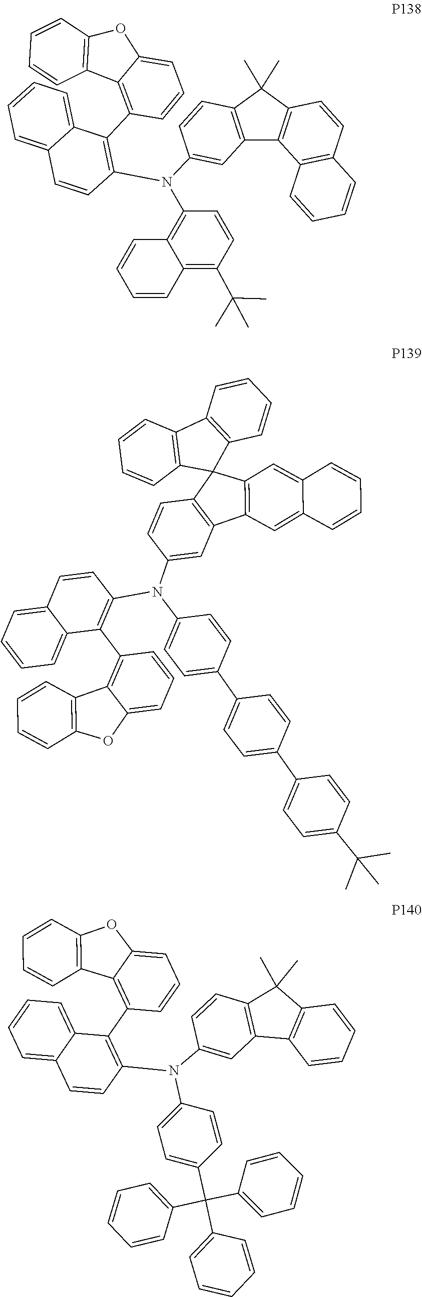 [SMILES: CC(C)(C)c1ccc(N(c2ccc3c(c2)-c2c(ccc4ccccc24)C3(C)C)c2ccc3ccccc3c2-c2cccc3oc4ccccc4c23)c2ccccc12 ; CC(C)(C)c1ccc(-c2ccc(-c3ccc(N(c4ccc5c(c4)-c4cc6ccccc6cc4C54c5ccccc5-c5ccccc54)c4ccc5ccccc5c4-c4cccc5oc6ccccc6c45)cc3)cc2)cc1 ; CC1(C)c2ccccc2-c2cc(N(c3ccc(C(c4ccccc4)(c4ccccc4)c4ccccc4)cc3)c3ccc4ccccc4c3-c3cccc4oc5ccccc5c34)ccc21]

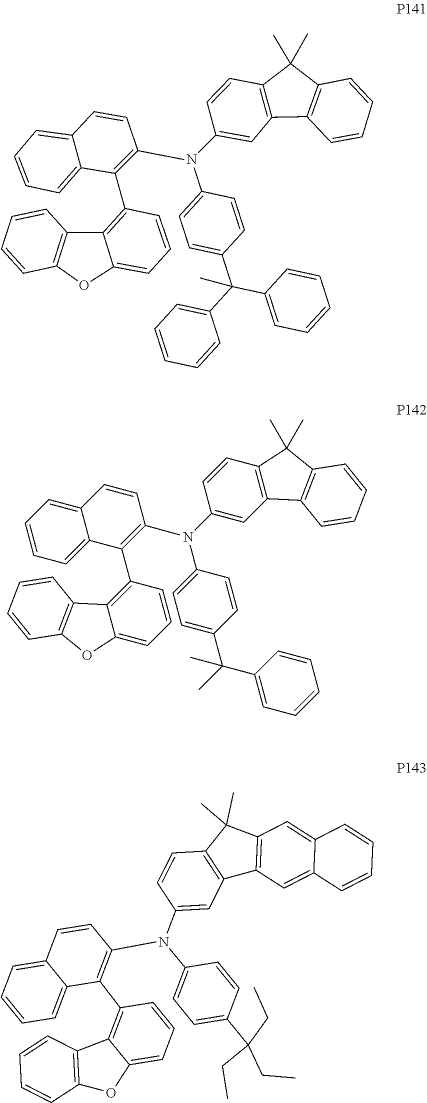 [SMILES: CC1(C)c2ccccc2-c2cc(N(c3ccc(C(C)(c4ccccc4)c4ccccc4)cc3)c3ccc4ccccc4c3-c3cccc4oc5ccccc5c34)ccc21 ; CC(C)(c1ccccc1)c1ccc(N(c2ccc3c(c2)-c2ccccc2C3(C)C)c2ccc3ccccc3c2-c2cccc3oc4ccccc4c23)cc1 ; CCC(CC)(CC)c1ccc(N(c2ccc3c(c2)-c2cc4ccccc4cc2C3(C)C)c2ccc3ccccc3c2-c2cccc3oc4ccccc4c23)cc1]

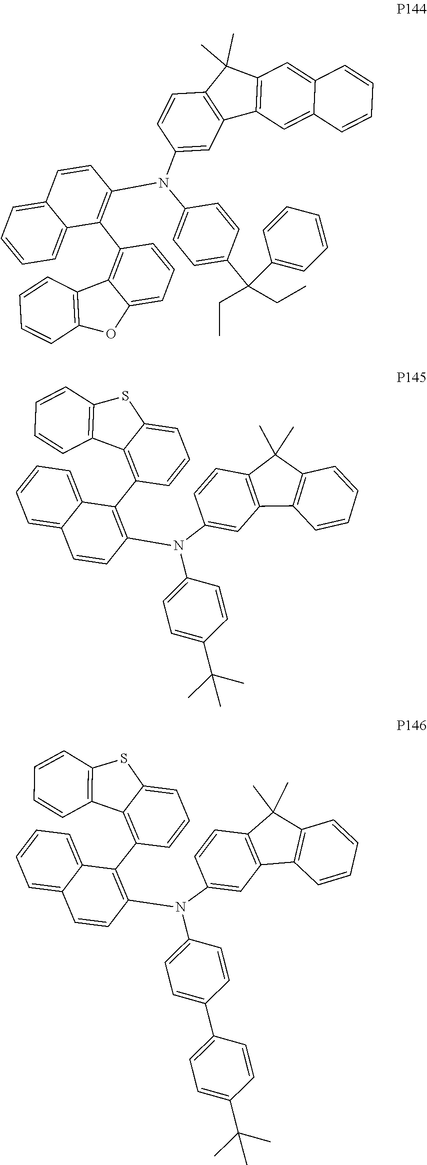

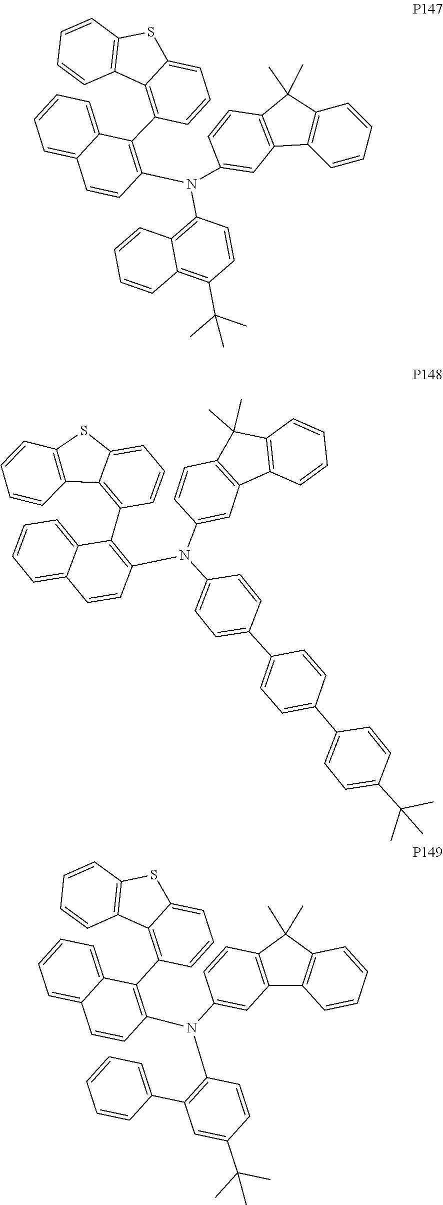 [SMILES: CC(C)(C)c1ccc(N(c2ccc3c(c2)-c2ccccc2C3(C)C)c2ccc3ccccc3c2-c2cccc3sc4ccccc4c23)c2ccccc12 ; CC(C)(C)c1ccc(-c2ccc(-c3ccc(N(c4ccc5c(c4)-c4ccccc4C5(C)C)c4ccc5ccccc5c4-c4cccc5sc6ccccc6c45)cc3)cc2)cc1 ; CC(C)(C)c1ccc(N(c2ccc3c(c2)-c2ccccc2C3(C)C)c2ccc3ccccc3c2-c2cccc3sc4ccccc4c23)c(-c2ccccc2)c1]

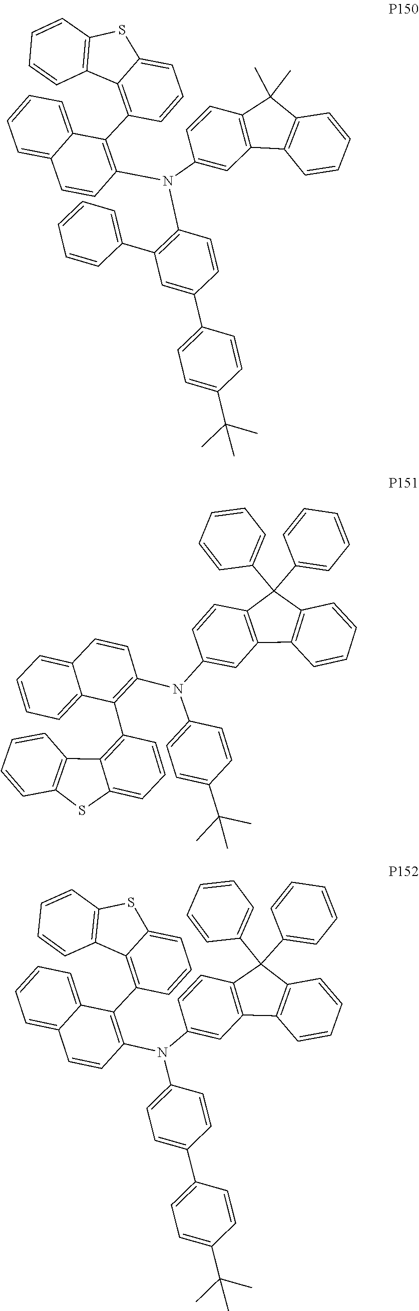

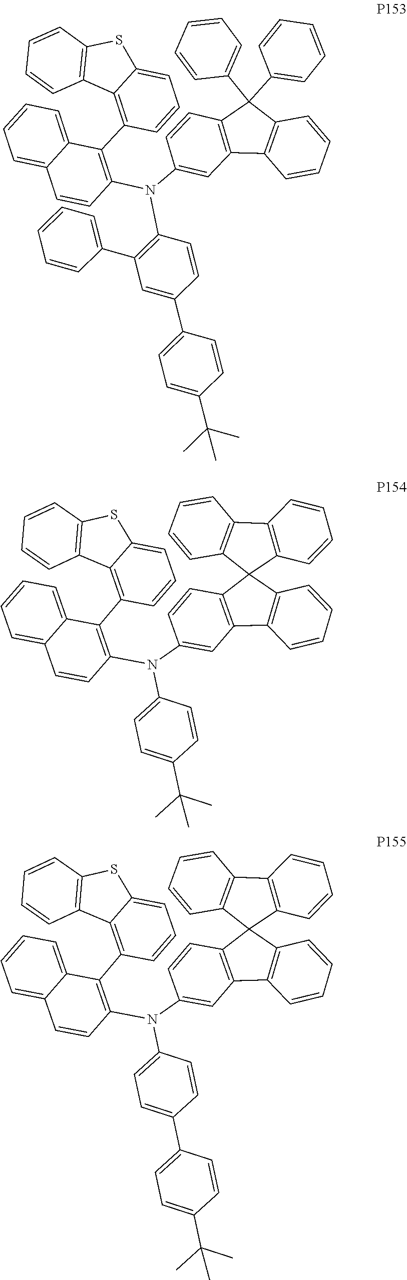

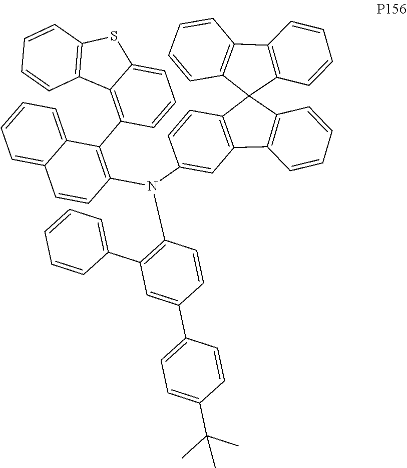 [SMILES: CC(C)(C)c1ccc(-c2ccc(N(c3ccc4c(c3)-c3ccccc3C43c4ccccc4-c4ccccc43)c3ccc4ccccc4c3-c3cccc4sc5ccccc5c34)c(-c3ccccc3)c2)cc1]

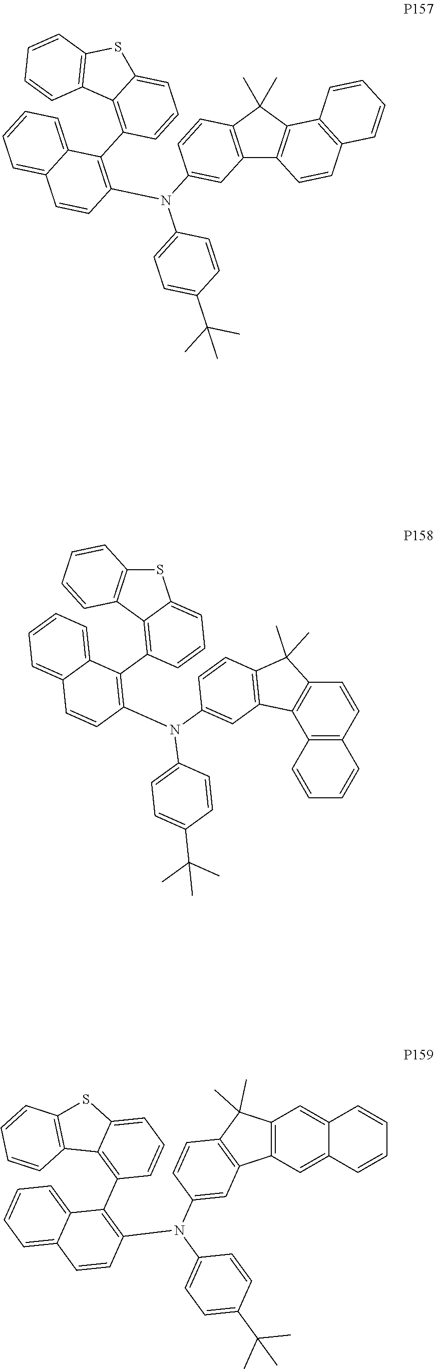 [SMILES: CC(C)(C)c1ccc(N(c2ccc3c(c2)-c2ccc4ccccc4c2C3(C)C)c2ccc3ccccc3c2-c2cccc3sc4ccccc4c23)cc1 ; CC(C)(C)c1ccc(N(c2ccc3c(c2)-c2c(ccc4ccccc24)C3(C)C)c2ccc3ccccc3c2-c2cccc3sc4ccccc4c23)cc1 ; CC(C)(C)c1ccc(N(c2ccc3c(c2)-c2cc4ccccc4cc2C3(C)C)c2ccc3ccccc3c2-c2cccc3sc4ccccc4c23)cc1]

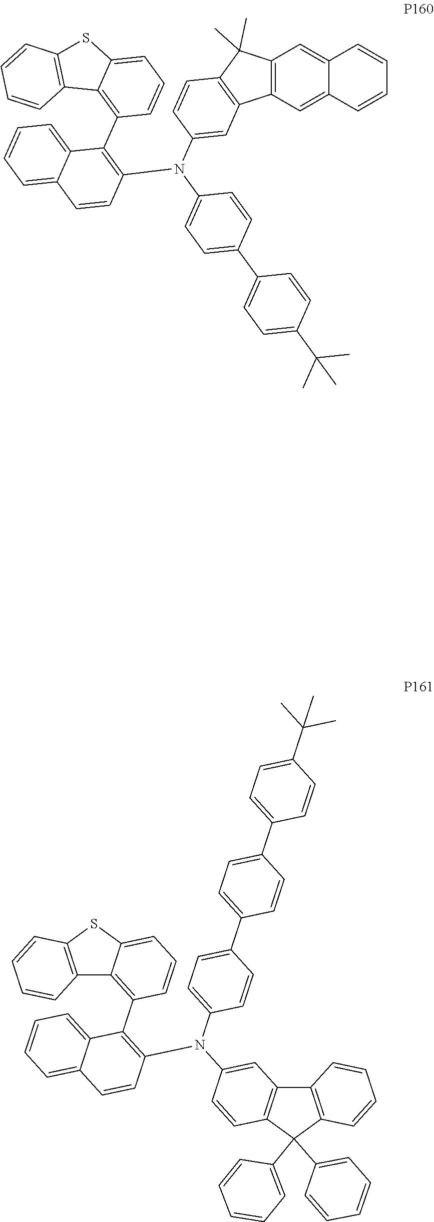 [SMILES: CC(C)(C)c1ccc(-c2ccc(N(c3ccc4c(c3)-c3cc5ccccc5cc3C4(C)C)c3ccc4ccccc4c3-c3cccc4sc5ccccc5c34)cc2)cc1 ; CC(C)(C)c1ccc(-c2ccc(-c3ccc(N(c4ccc5c(c4)-c4ccccc4C5(c4ccccc4)c4ccccc4)c4ccc5ccccc5c4-c4cccc5sc6ccccc6c45)cc3)cc2)cc1]

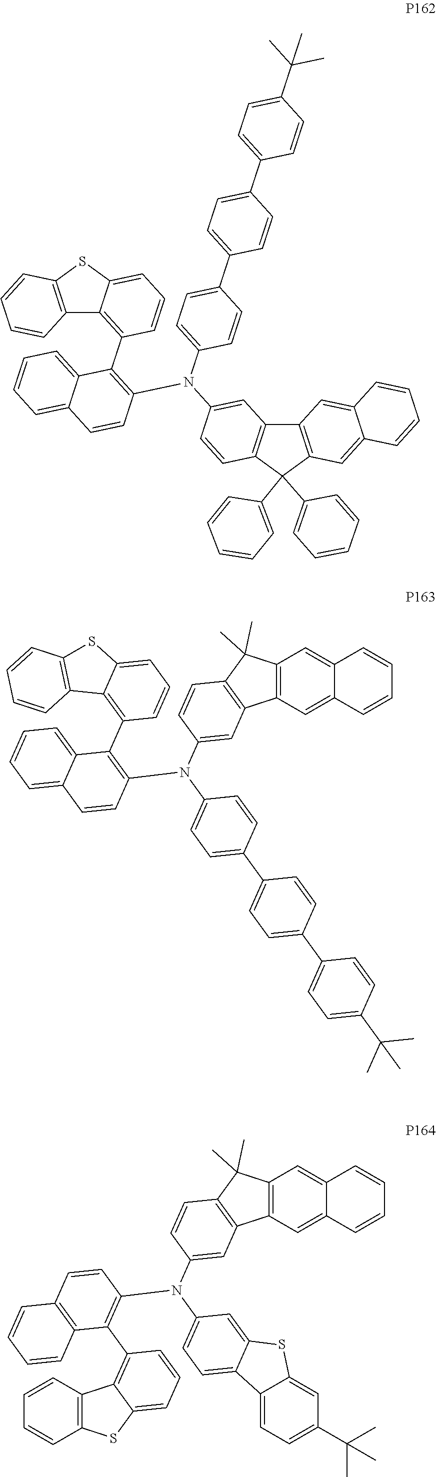 [SMILES: CC(C)(C)c1ccc(-c2ccc(-c3ccc(N(c4ccc5c(c4)-c4cc6ccccc6cc4C5(c4ccccc4)c4ccccc4)c4ccc5ccccc5c4-c4cccc5sc6ccccc6c45)cc3)cc2)cc1 ; CC(C)(C)c1ccc(-c2ccc(-c3ccc(N(c4ccc5c(c4)-c4cc6ccccc6cc4C5(C)C)c4ccc5ccccc5c4-c4cccc5sc6ccccc6c45)cc3)cc2)cc1 ; CC(C)(C)c1ccc2c(c1)sc1cc(N(c3ccc4c(c3)-c3cc5ccccc5cc3C4(C)C)c3ccc4ccccc4c3-c3cccc4sc5ccccc5c34)ccc12]

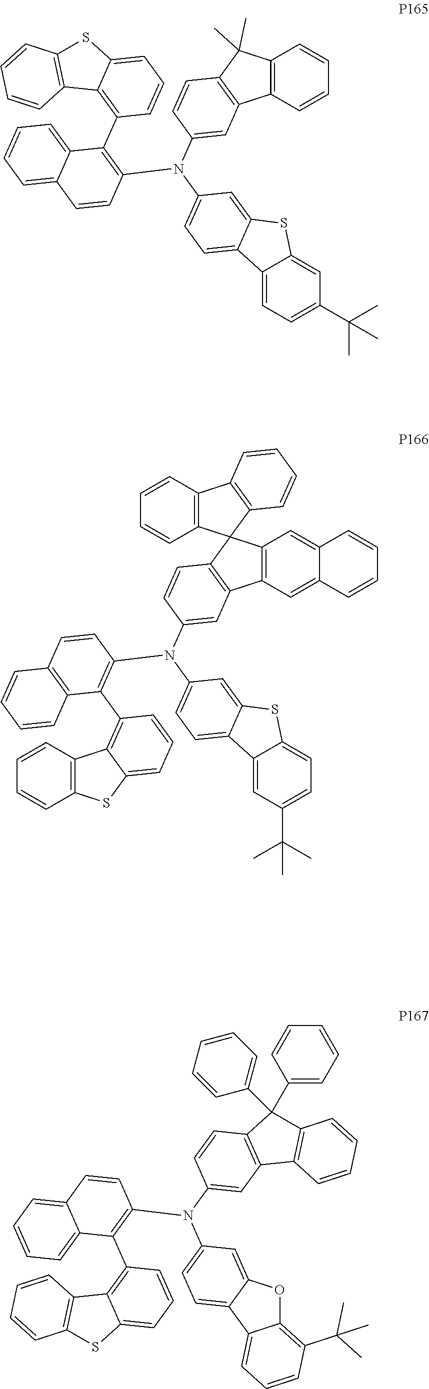 [SMILES: CC(C)(C)c1ccc2c(c1)sc1cc(N(c3ccc4c(c3)-c3ccccc3C4(C)C)c3ccc4ccccc4c3-c3cccc4sc5ccccc5c34)ccc12 ; CC(C)(C)c1ccc2sc3cc(N(c4ccc5c(c4)-c4cc6ccccc6cc4C54c5ccccc5-c5ccccc54)c4ccc5ccccc5c4-c4cccc5sc6ccccc6c45)ccc3c2c1 ; CC(C)(C)c1cccc2c1oc1cc(N(c3ccc4c(c3)-c3ccccc3C4(c3ccccc3)c3ccccc3)c3ccc4ccccc4c3-c3cccc4sc5ccccc5c34)ccc12]

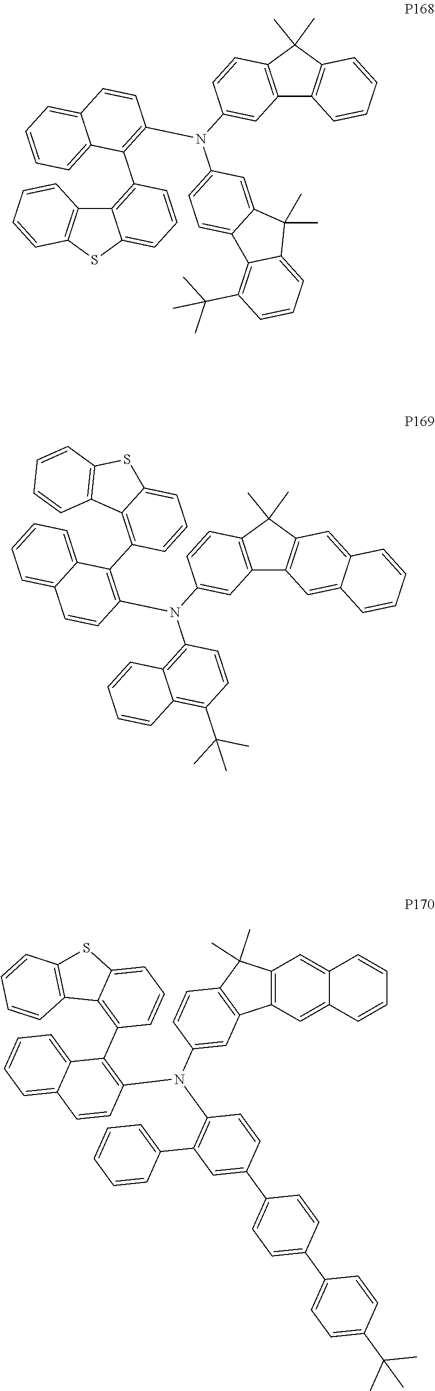 [SMILES: CC(C)(C)c1cccc2c1-c1ccc(N(c3ccc4c(c3)-c3ccccc3C4(C)C)c3ccc4ccccc4c3-c3cccc4sc5ccccc5c34)cc1C2(C)C ; CC(C)(C)c1ccc(N(c2ccc3c(c2)-c2cc4ccccc4cc2C3(C)C)c2ccc3ccccc3c2-c2cccc3sc4ccccc4c23)c2ccccc12 ; CC(C)(C)c1ccc(-c2ccc(-c3ccc(N(c4ccc5c(c4)-c4cc6ccccc6cc4C5(C)C)c4ccc5ccccc5c4-c4cccc5sc6ccccc6c45)c(-c4ccccc4)c3)cc2)cc1]

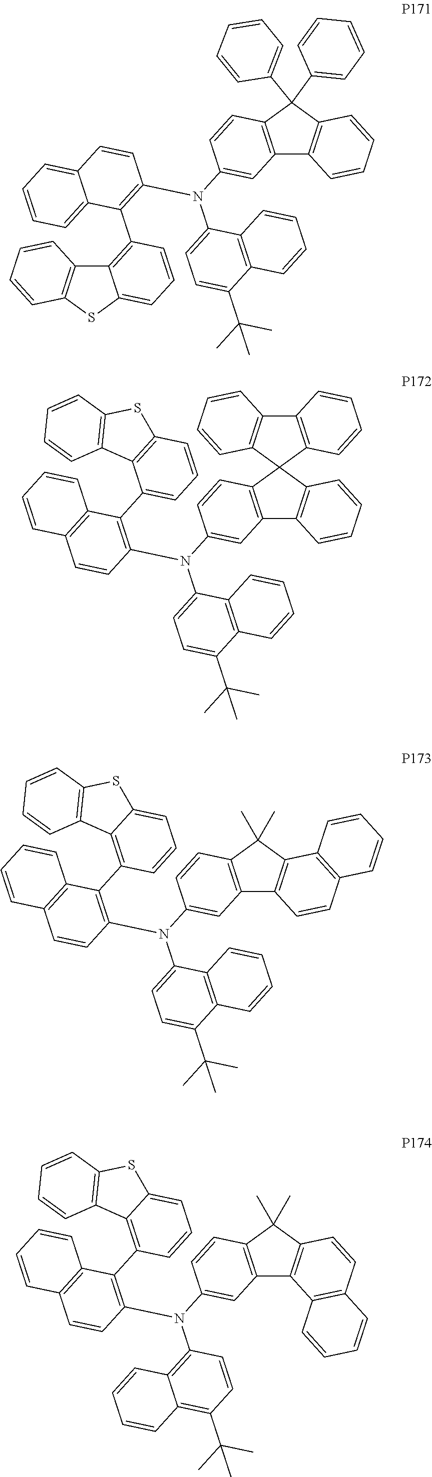

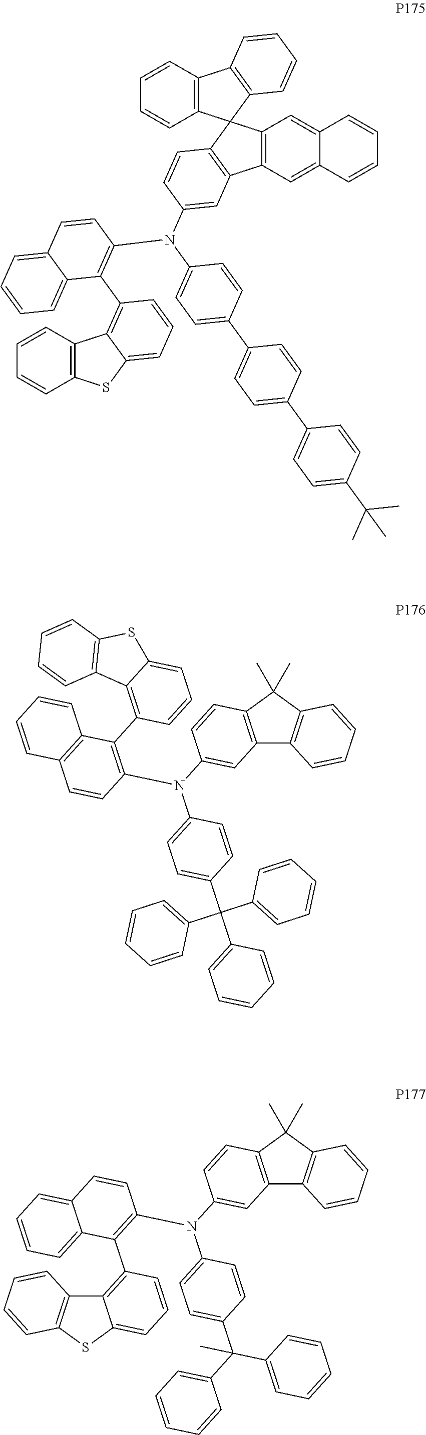 [SMILES: CC(C)(C)c1ccc(-c2ccc(-c3ccc(N(c4ccc5c(c4)-c4cc6ccccc6cc4C54c5ccccc5-c5ccccc54)c4ccc5ccccc5c4-c4cccc5sc6ccccc6c45)cc3)cc2)cc1 ; CC1(C)c2ccccc2-c2cc(N(c3ccc(C(c4ccccc4)(c4ccccc4)c4ccccc4)cc3)c3ccc4ccccc4c3-c3cccc4sc5ccccc5c34)ccc21 ; CC1(C)c2ccccc2-c2cc(N(c3ccc(C(C)(c4ccccc4)c4ccccc4)cc3)c3ccc4ccccc4c3-c3cccc4sc5ccccc5c34)ccc21]

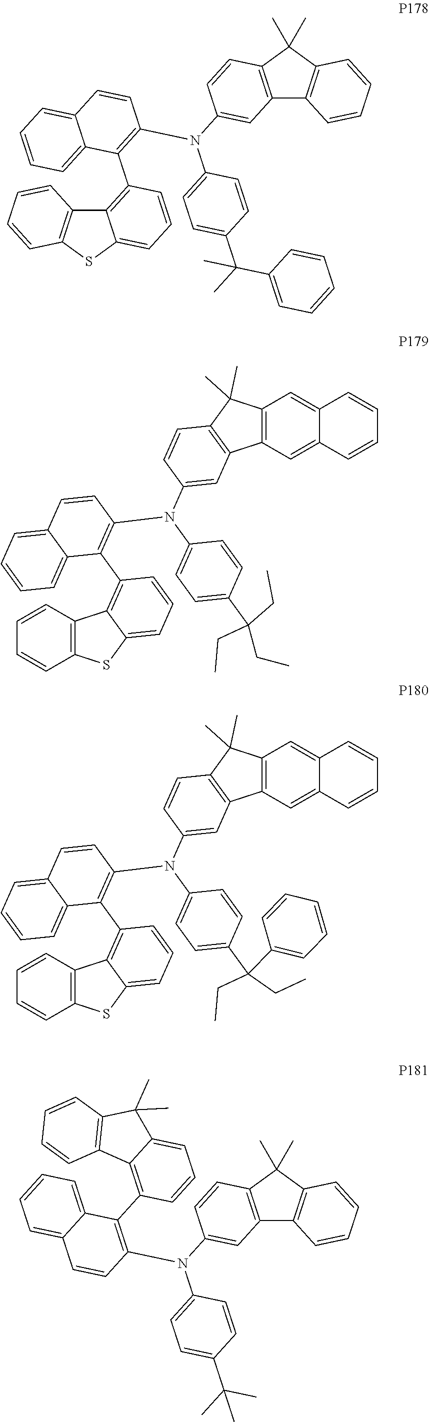 [SMILES: CC(C)(c1ccccc1)c1ccc(N(c2ccc3c(c2)-c2ccccc2C3(C)C)c2ccc3ccccc3c2-c2cccc3sc4ccccc4c23)cc1 ; CCC(CC)(CC)c1ccc(N(c2ccc3c(c2)-c2cc4ccccc4cc2C3(C)C)c2ccc3ccccc3c2-c2cccc3sc4ccccc4c23)cc1 ; CCC(CC)(c1ccccc1)c1ccc(N(c2ccc3c(c2)-c2cc4ccccc4cc2C3(C)C)c2ccc3ccccc3c2-c2cccc3sc4ccccc4c23)cc1 ; CC(C)(C)c1ccc(N(c2ccc3c(c2)-c2ccccc2C3(C)C)c2ccc3ccccc3c2-c2cccc3c2-c2ccccc2C3(C)C)cc1]

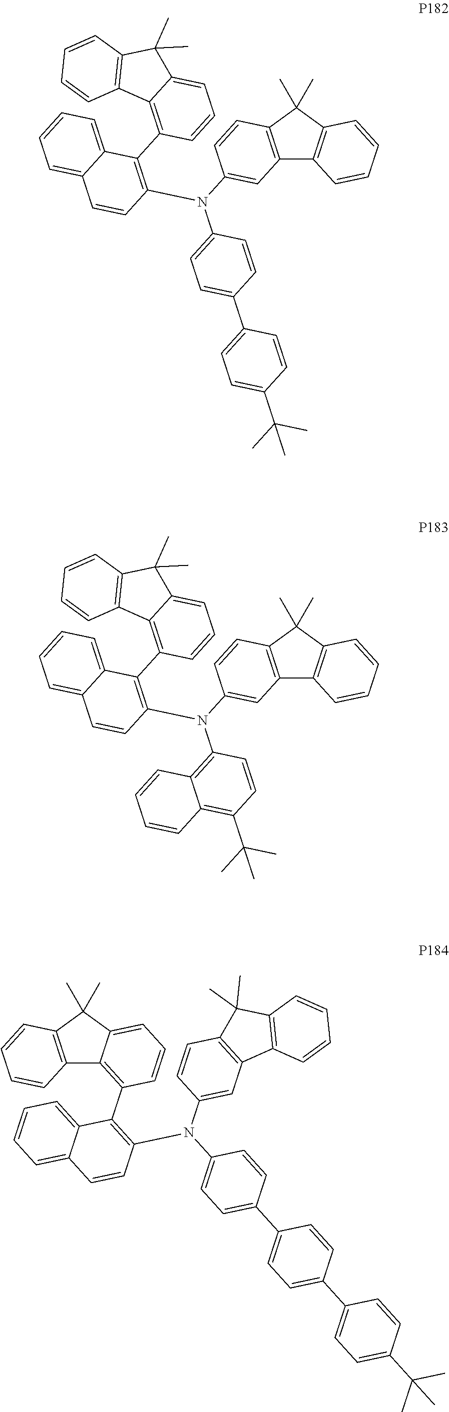 [SMILES: CC(C)(C)c1ccc(-c2ccc(N(c3ccc4c(c3)-c3ccccc3C4(C)C)c3ccc4ccccc4c3-c3cccc4c3-c3ccccc3C4(C)C)cc2)cc1 ; CC(C)(C)c1ccc(N(c2ccc3c(c2)-c2ccccc2C3(C)C)c2ccc3ccccc3c2-c2cccc3c2-c2ccccc2C3(C)C)c2ccccc12 ; CC(C)(C)c1ccc(-c2ccc(-c3ccc(N(c4ccc5c(c4)-c4ccccc4C5(C)C)c4ccc5ccccc5c4-c4cccc5c4-c4ccccc4C5(C)C)cc3)cc2)cc1]

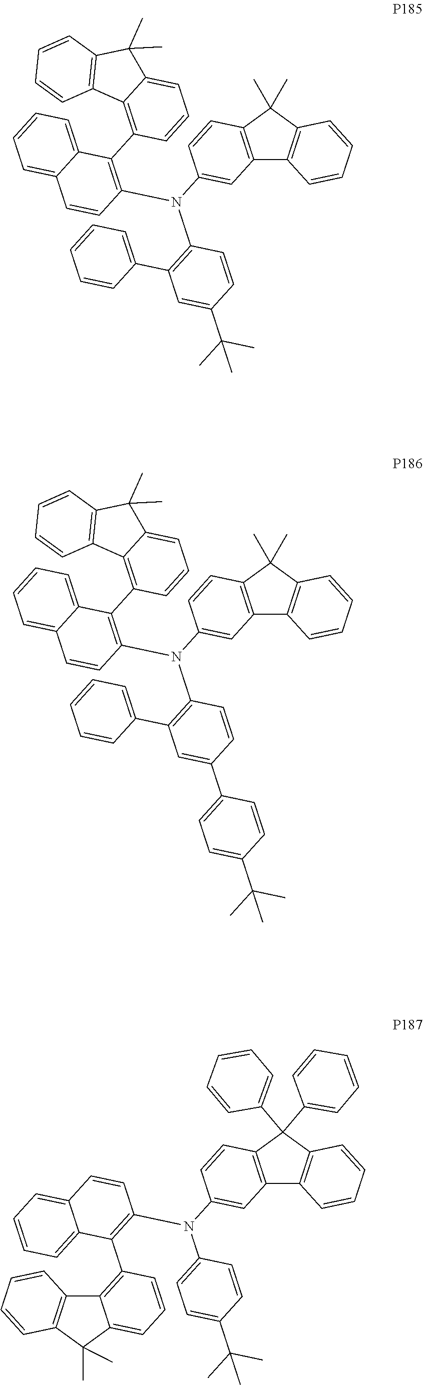 [SMILES: CC(C)(C)c1ccc(N(c2ccc3c(c2)-c2ccccc2C3(C)C)c2ccc3ccccc3c2-c2cccc3c2-c2ccccc2C3(C)C)c(-c2ccccc2)c1 ; CC(C)(C)c1ccc(-c2ccc(N(c3ccc4c(c3)-c3ccccc3C4(C)C)c3ccc4ccccc4c3-c3cccc4c3-c3ccccc3C4(C)C)c(-c3ccccc3)c2)cc1 ; CC(C)(C)c1ccc(N(c2ccc3c(c2)-c2ccccc2C3(c2ccccc2)c2ccccc2)c2ccc3ccccc3c2-c2cccc3c2-c2ccccc2C3(C)C)cc1]

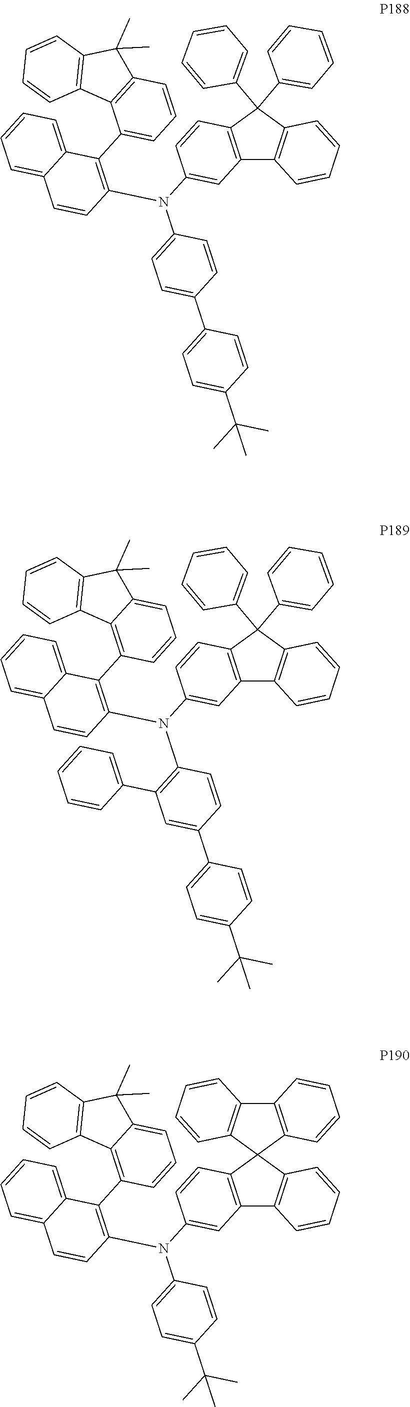 [SMILES: CC(C)(C)c1ccc(-c2ccc(N(c3ccc4c(c3)-c3ccccc3C4(c3ccccc3)c3ccccc3)c3ccc4ccccc4c3-c3cccc4c3-c3ccccc3C4(C)C)cc2)cc1 ; CC(C)(C)c1ccc(-c2ccc(N(c3ccc4c(c3)-c3ccccc3C4(c3ccccc3)c3ccccc3)c3ccc4ccccc4c3-c3cccc4c3-c3ccccc3C4(C)C)c(-c3ccccc3)c2)cc1 ; CC(C)(C)c1ccc(N(c2ccc3c(c2)-c2ccccc2C32c3ccccc3-c3ccccc32)c2ccc3ccccc3c2-c2cccc3c2-c2ccccc2C3(C)C)cc1]

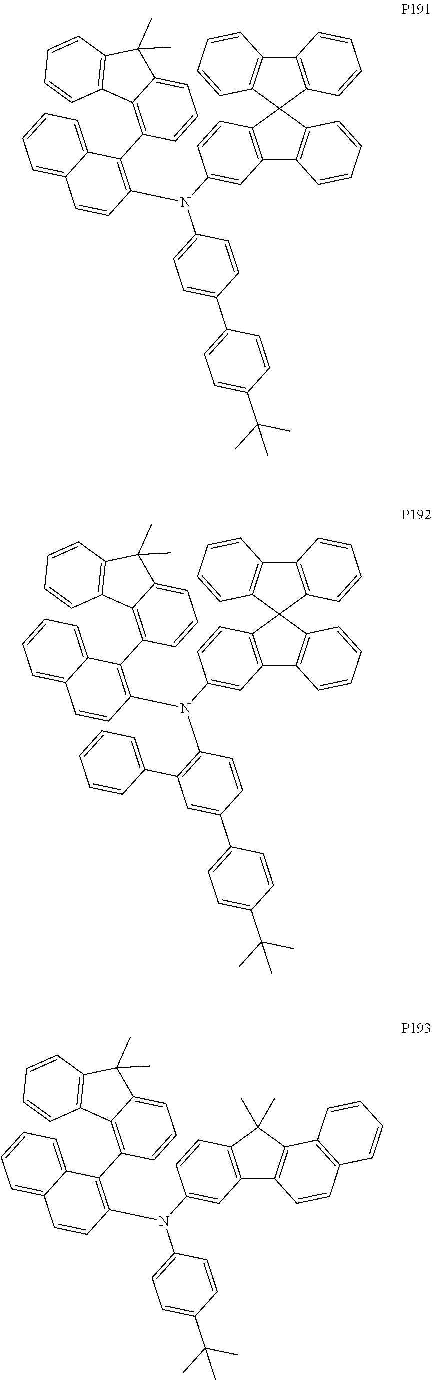 [SMILES: CC(C)(C)c1ccc(-c2ccc(N(c3ccc4c(c3)-c3ccccc3C43c4ccccc4-c4ccccc43)c3ccc4ccccc4c3-c3cccc4c3-c3ccccc3C4(C)C)cc2)cc1 ; CC(C)(C)c1ccc(-c2ccc(N(c3ccc4c(c3)-c3ccccc3C43c4ccccc4-c4ccccc43)c3ccc4ccccc4c3-c3cccc4c3-c3ccccc3C4(C)C)c(-c3ccccc3)c2)cc1 ; CC(C)(C)c1ccc(N(c2ccc3c(c2)-c2ccc4ccccc4c2C3(C)C)c2ccc3ccccc3c2-c2cccc3c2-c2ccccc2C3(C)C)cc1]

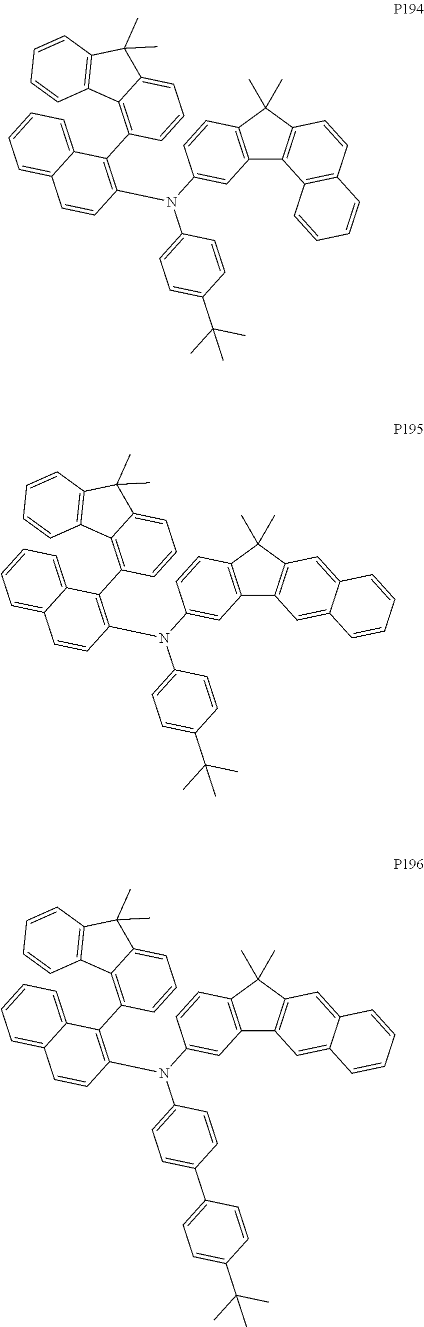 [SMILES: CC(C)(C)c1ccc(N(c2ccc3c(c2)-c2c(ccc4ccccc24)C3(C)C)c2ccc3ccccc3c2-c2cccc3c2-c2ccccc2C3(C)C)cc1 ; CC(C)(C)c1ccc(N(c2ccc3c(c2)-c2cc4ccccc4cc2C3(C)C)c2ccc3ccccc3c2-c2cccc3c2-c2ccccc2C3(C)C)cc1 ; CC(C)(C)c1ccc(-c2ccc(N(c3ccc4c(c3)-c3cc5ccccc5cc3C4(C)C)c3ccc4ccccc4c3-c3cccc4c3-c3ccccc3C4(C)C)cc2)cc1]

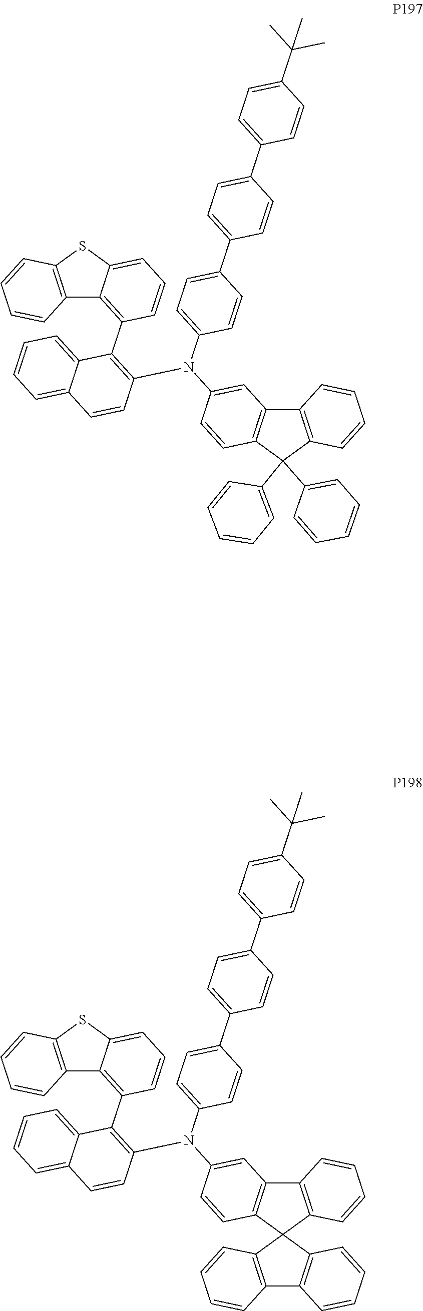 [SMILES: CC(C)(C)c1ccc(-c2ccc(-c3ccc(N(c4ccc5c(c4)-c4ccccc4C5(c4ccccc4)c4ccccc4)c4ccc5ccccc5c4-c4cccc5sc6ccccc6c45)cc3)cc2)cc1 ; CC(C)(C)c1ccc(-c2ccc(-c3ccc(N(c4ccc5c(c4)-c4ccccc4C54c5ccccc5-c5ccccc54)c4ccc5ccccc5c4-c4cccc5sc6ccccc6c45)cc3)cc2)cc1]

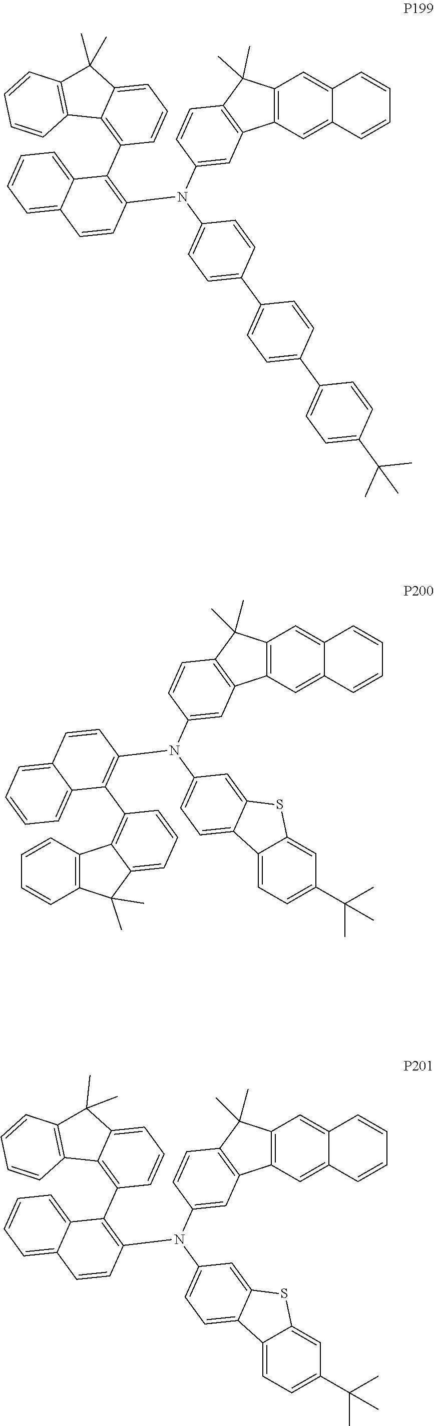 [SMILES: CC(C)(C)c1ccc(-c2ccc(-c3ccc(N(c4ccc5c(c4)-c4cc6ccccc6cc4C5(C)C)c4ccc5ccccc5c4-c4cccc5c4-c4ccccc4C5(C)C)cc3)cc2)cc1 ; CC(C)(C)c1ccc2c(c1)sc1cc(N(c3ccc4c(c3)-c3cc5ccccc5cc3C4(C)C)c3ccc4ccccc4c3-c3cccc4c3-c3ccccc3C4(C)C)ccc12 ; CC(C)(C)c1ccc2c(c1)sc1cc(N(c3ccc4c(c3)-c3cc5ccccc5cc3C4(C)C)c3ccc4ccccc4c3-c3cccc4c3-c3ccccc3C4(C)C)ccc12]

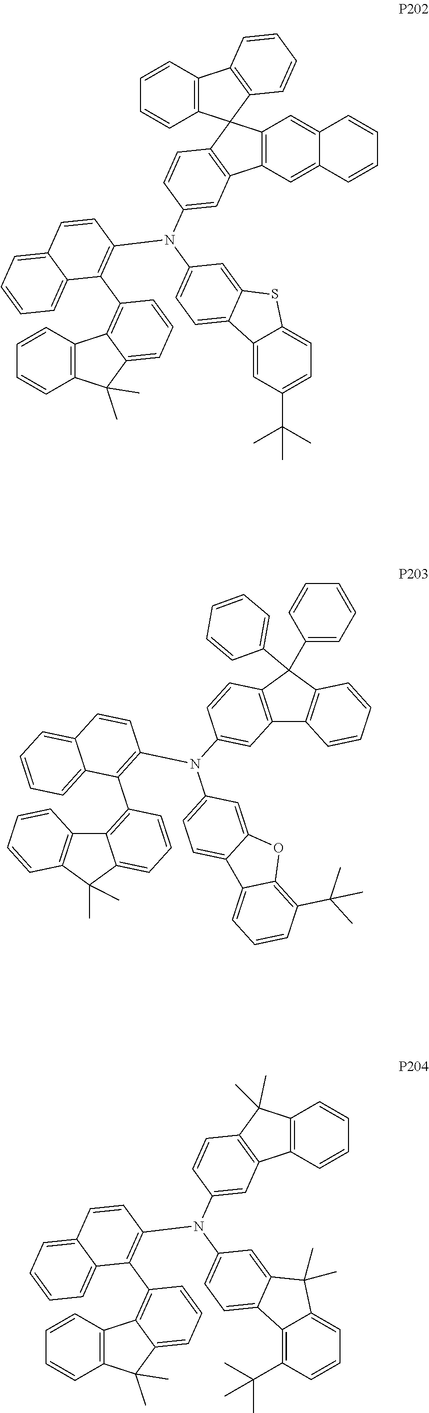 [SMILES: CC(C)(C)c1ccc2sc3cc(N(c4ccc5c(c4)-c4cc6ccccc6cc4C54c5ccccc5-c5ccccc54)c4ccc5ccccc5c4-c4cccc5c4-c4ccccc4C5(C)C)ccc3c2c1 ; CC(C)(C)c1cccc2c1oc1cc(N(c3ccc4c(c3)-c3ccccc3C4(c3ccccc3)c3ccccc3)c3ccc4ccccc4c3-c3cccc4c3-c3ccccc3C4(C)C)ccc12 ; CC(C)(C)c1cccc2c1-c1ccc(N(c3ccc4c(c3)-c3ccccc3C4(C)C)c3ccc4ccccc4c3-c3cccc4c3-c3ccccc3C4(C)C)cc1C2(C)C]

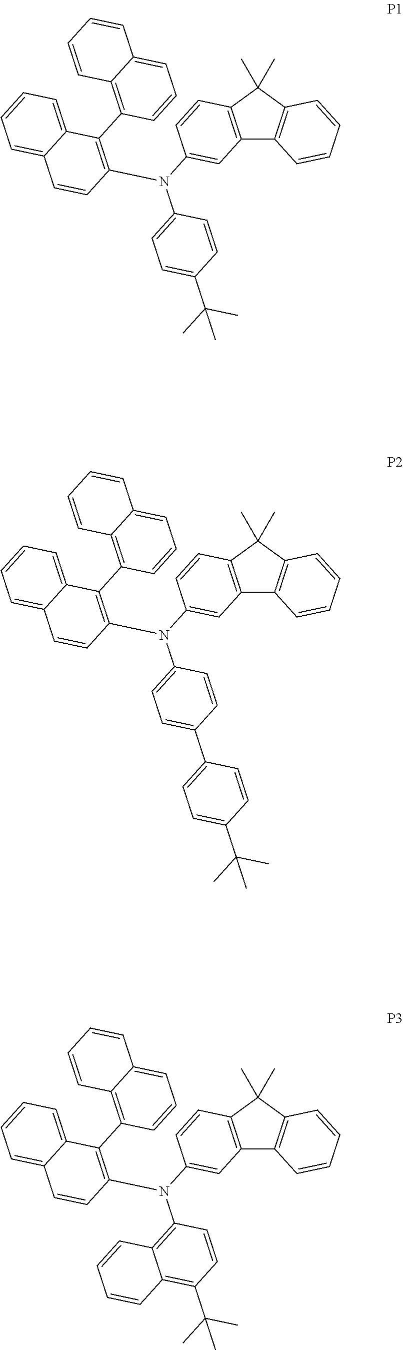 [SMILES: CC(C)(C)c1ccc(N(c2ccc3c(c2)-c2ccccc2C3(C)C)c2ccc3ccccc3c2-c2cccc3ccccc23)cc1 ; CC(C)(C)c1ccc(-c2ccc(N(c3ccc4c(c3)-c3ccccc3C4(C)C)c3ccc4ccccc4c3-c3cccc4ccccc34)cc2)cc1 ; CC(C)(C)c1ccc(N(c2ccc3c(c2)-c2ccccc2C3(C)C)c2ccc3ccccc3c2-c2cccc3ccccc23)c2ccccc12]

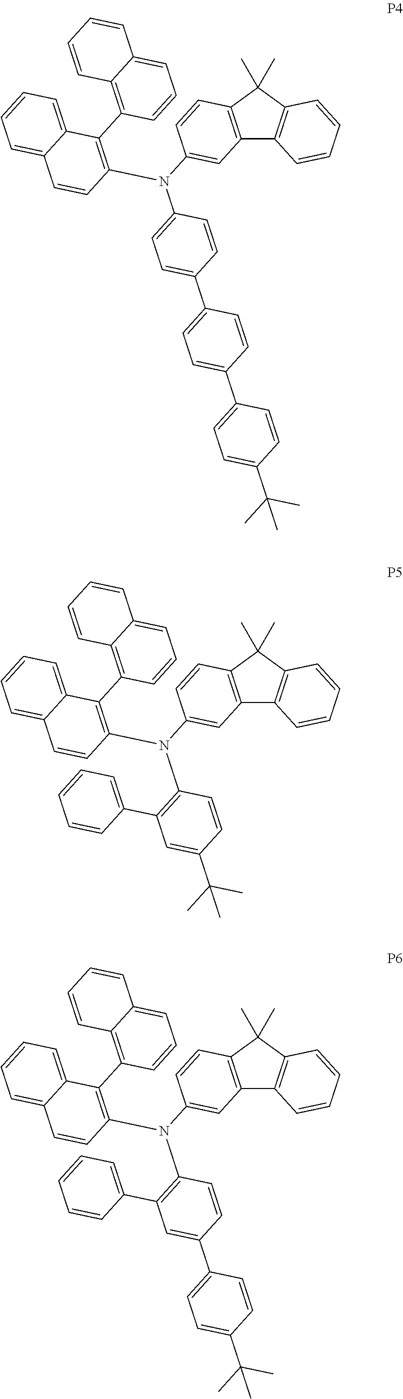 [SMILES: CC(C)(C)c1ccc(-c2ccc(-c3ccc(N(c4ccc5c(c4)-c4ccccc4C5(C)C)c4ccc5ccccc5c4-c4cccc5ccccc45)cc3)cc2)cc1 ; CC(C)(C)c1ccc(N(c2ccc3c(c2)-c2ccccc2C3(C)C)c2ccc3ccccc3c2-c2cccc3ccccc23)c(-c2ccccc2)c1 ; CC(C)(C)c1ccc(-c2ccc(N(c3ccc4c(c3)-c3ccccc3C4(C)C)c3ccc4ccccc4c3-c3cccc4ccccc34)c(-c3ccccc3)c2)cc1]

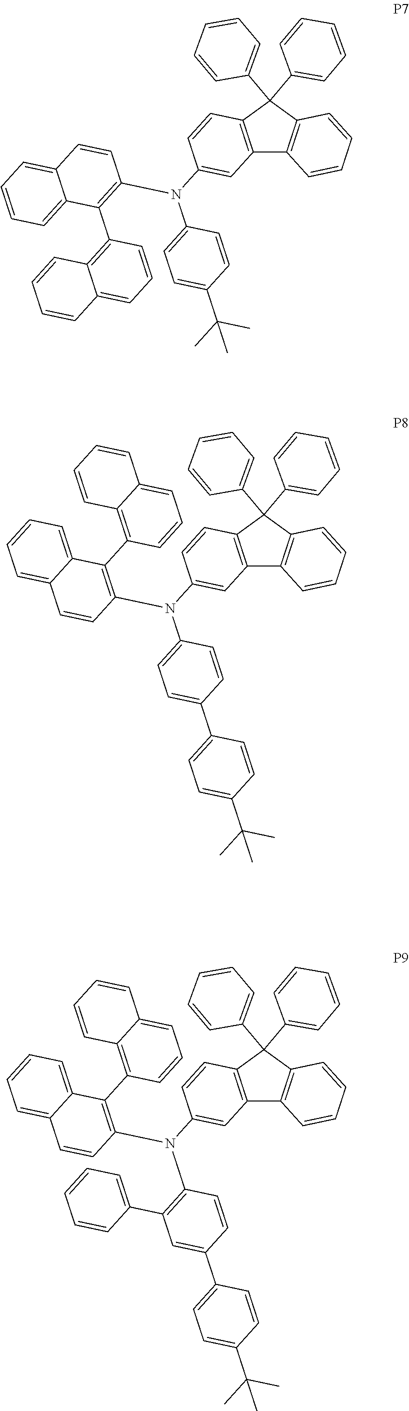 [SMILES: CC(C)(C)c1ccc(N(c2ccc3c(c2)-c2ccccc2C3(c2ccccc2)c2ccccc2)c2ccc3ccccc3c2-c2cccc3ccccc23)cc1 ; CC(C)(C)c1ccc(-c2ccc(N(c3ccc4c(c3)-c3ccccc3C4(c3ccccc3)c3ccccc3)c3ccc4ccccc4c3-c3cccc4ccccc34)cc2)cc1 ; CC(C)(C)c1ccc(-c2ccc(N(c3ccc4c(c3)-c3ccccc3C4(c3ccccc3)c3ccccc3)c3ccc4ccccc4c3-c3cccc4ccccc34)c(-c3ccccc3)c2)cc1]

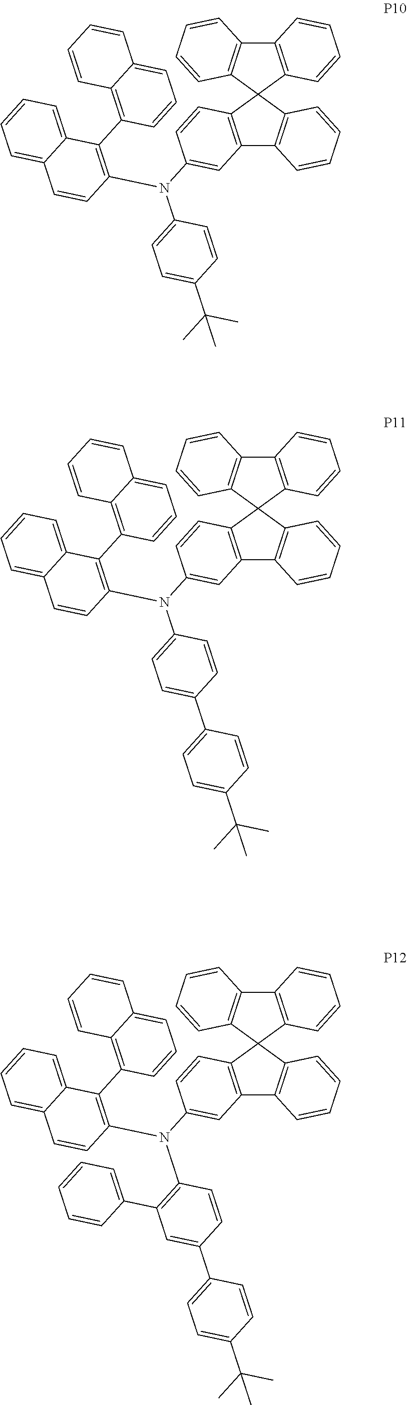 [SMILES: CC(C)(C)c1ccc(N(c2ccc3c(c2)-c2ccccc2C32c3ccccc3-c3ccccc32)c2ccc3ccccc3c2-c2cccc3ccccc23)cc1 ; CC(C)(C)c1ccc(-c2ccc(N(c3ccc4c(c3)-c3ccccc3C43c4ccccc4-c4ccccc43)c3ccc4ccccc4c3-c3cccc4ccccc34)cc2)cc1 ; CC(C)(C)c1ccc(-c2ccc(N(c3ccc4c(c3)-c3ccccc3C43c4ccccc4-c4ccccc43)c3ccc4ccccc4c3-c3cccc4ccccc34)c(-c3ccccc3)c2)cc1]

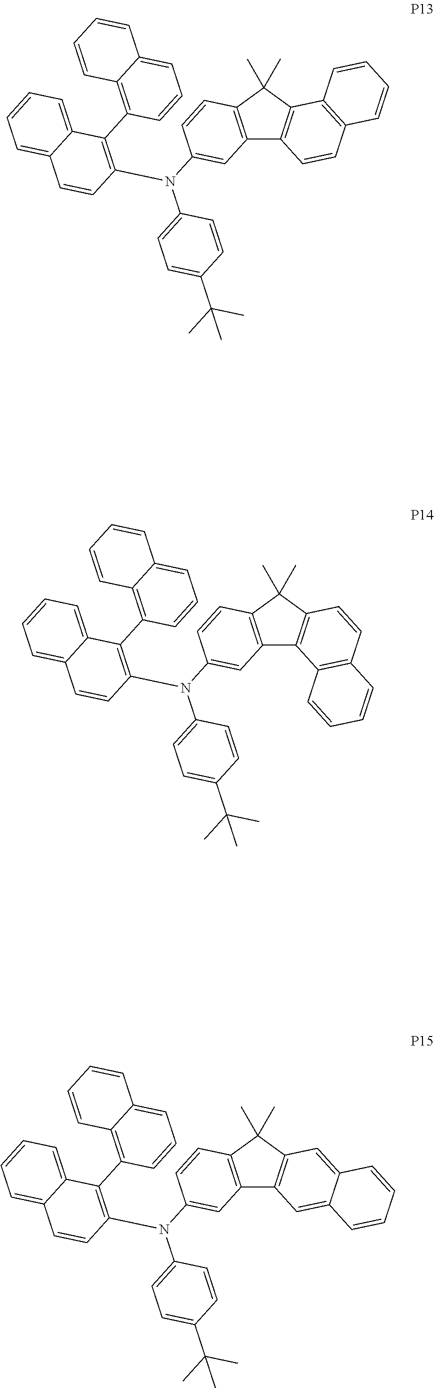 [SMILES: CC(C)(C)c1ccc(N(c2ccc3c(c2)-c2ccc4ccccc4c2C3(C)C)c2ccc3ccccc3c2-c2cccc3ccccc23)cc1 ; CC(C)(C)c1ccc(N(c2ccc3c(c2)-c2c(ccc4ccccc24)C3(C)C)c2ccc3ccccc3c2-c2cccc3ccccc23)cc1 ; CC(C)(C)c1ccc(N(c2ccc3c(c2)-c2cc4ccccc4cc2C3(C)C)c2ccc3ccccc3c2-c2cccc3ccccc23)cc1]

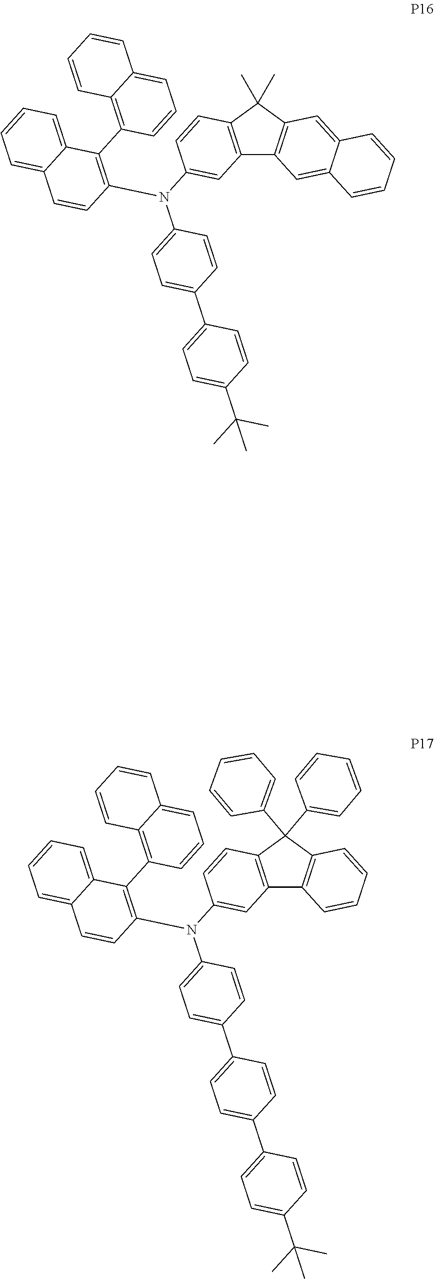 [SMILES: CC(C)(C)c1ccc(-c2ccc(N(c3ccc4c(c3)-c3cc5ccccc5cc3C4(C)C)c3ccc4ccccc4c3-c3cccc4ccccc34)cc2)cc1 ; CC(C)(C)c1ccc(-c2ccc(-c3ccc(N(c4ccc5c(c4)-c4ccccc4C5(c4ccccc4)c4ccccc4)c4ccc5ccccc5c4-c4cccc5ccccc45)cc3)cc2)cc1]

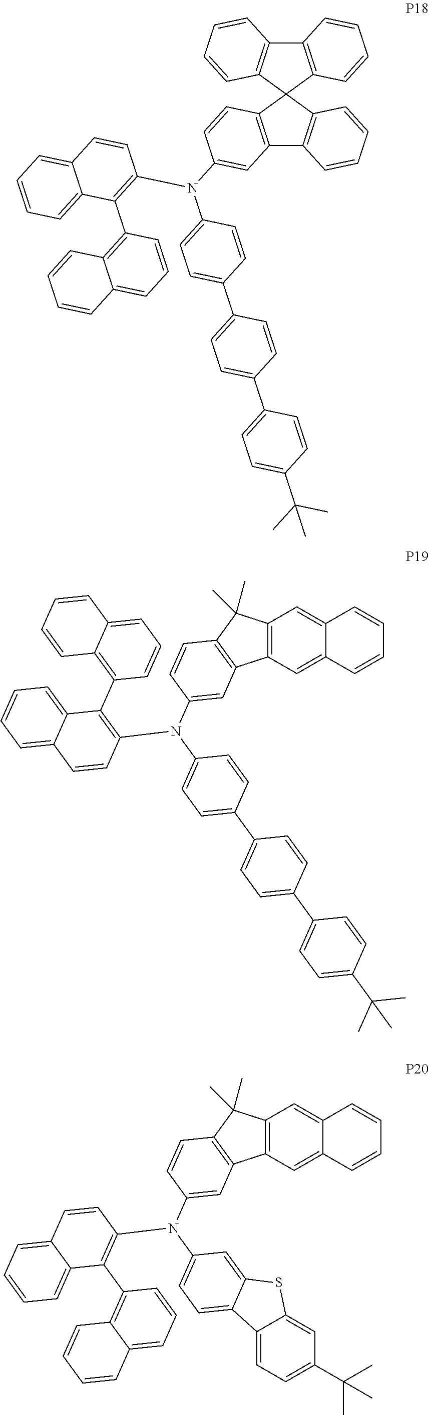 [SMILES: CC(C)(C)c1ccc(-c2ccc(-c3ccc(N(c4ccc5c(c4)-c4ccccc4C54c5ccccc5-c5ccccc54)c4ccc5ccccc5c4-c4cccc5ccccc45)cc3)cc2)cc1 ; CC(C)(C)c1ccc(-c2ccc(-c3ccc(N(c4ccc5c(c4)-c4cc6ccccc6cc4C5(C)C)c4ccc5ccccc5c4-c4cccc5ccccc45)cc3)cc2)cc1 ; CC(C)(C)c1ccc2c(c1)sc1cc(N(c3ccc4c(c3)-c3cc5ccccc5cc3C4(C)C)c3ccc4ccccc4c3-c3cccc4ccccc34)ccc12]

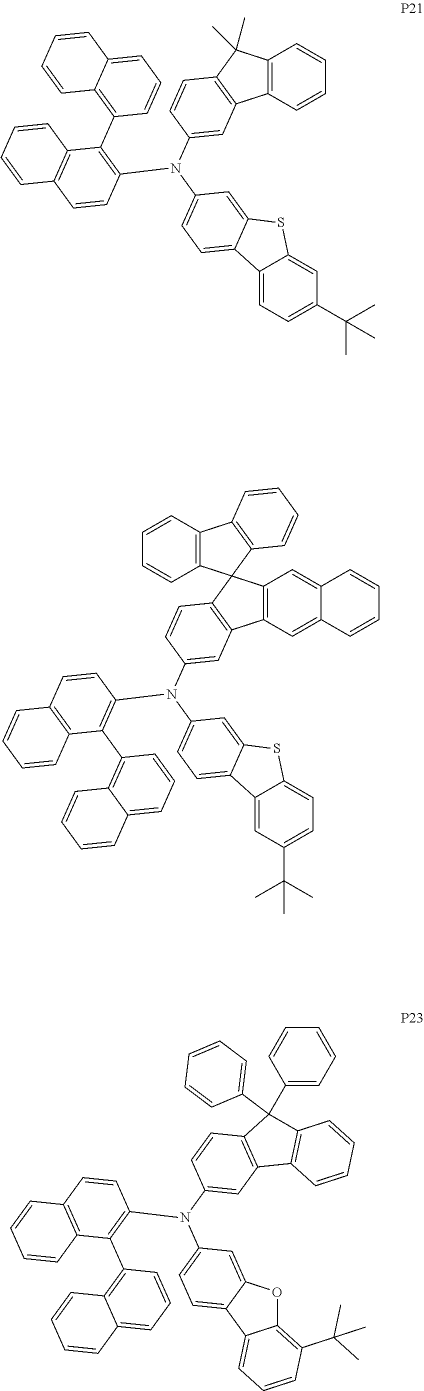 [SMILES: CC(C)(C)c1ccc2c(c1)sc1cc(N(c3ccc4c(c3)-c3ccccc3C4(C)C)c3ccc4ccccc4c3-c3cccc4ccccc34)ccc12 ; CC(C)(C)c1ccc2sc3cc(N(c4ccc5c(c4)-c4cc6ccccc6cc4C54c5ccccc5-c5ccccc54)c4ccc5ccccc5c4-c4cccc5ccccc45)ccc3c2c1 ; CC(C)(C)c1cccc2c1oc1cc(N(c3ccc4c(c3)-c3ccccc3C4(c3ccccc3)c3ccccc3)c3ccc4ccccc4c3-c3cccc4ccccc34)ccc12]

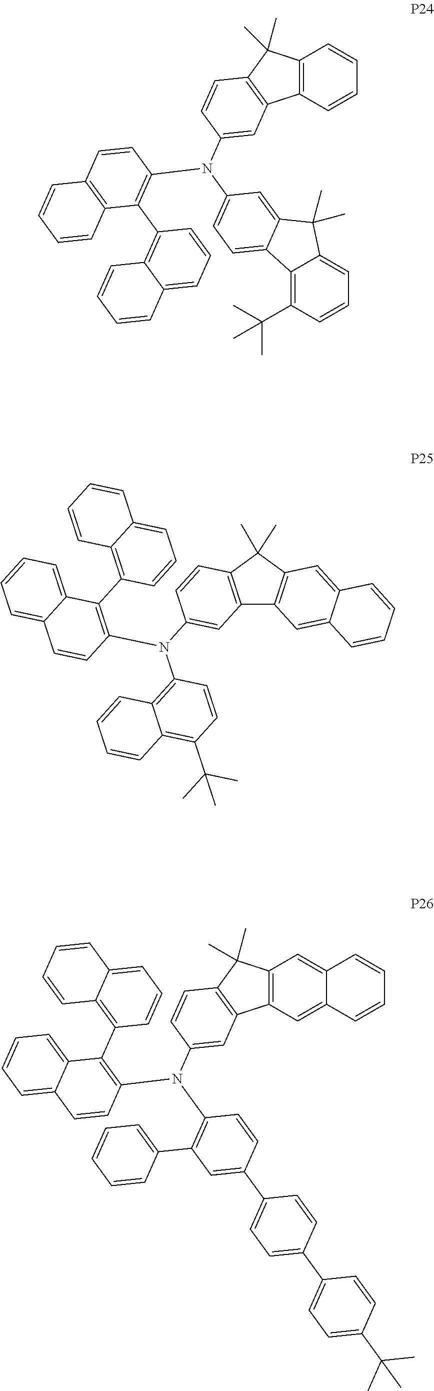 [SMILES: CC(C)(C)c1cccc2c1-c1ccc(N(c3ccc4c(c3)-c3ccccc3C4(C)C)c3ccc4ccccc4c3-c3cccc4ccccc34)cc1C2(C)C ; CC(C)(C)c1ccc(N(c2ccc3c(c2)-c2cc4ccccc4cc2C3(C)C)c2ccc3ccccc3c2-c2cccc3ccccc23)c2ccccc12 ; CC(C)(C)c1ccc(-c2ccc(-c3ccc(N(c4ccc5c(c4)-c4cc6ccccc6cc4C5(C)C)c4ccc5ccccc5c4-c4cccc5ccccc45)c(-c4ccccc4)c3)cc2)cc1]

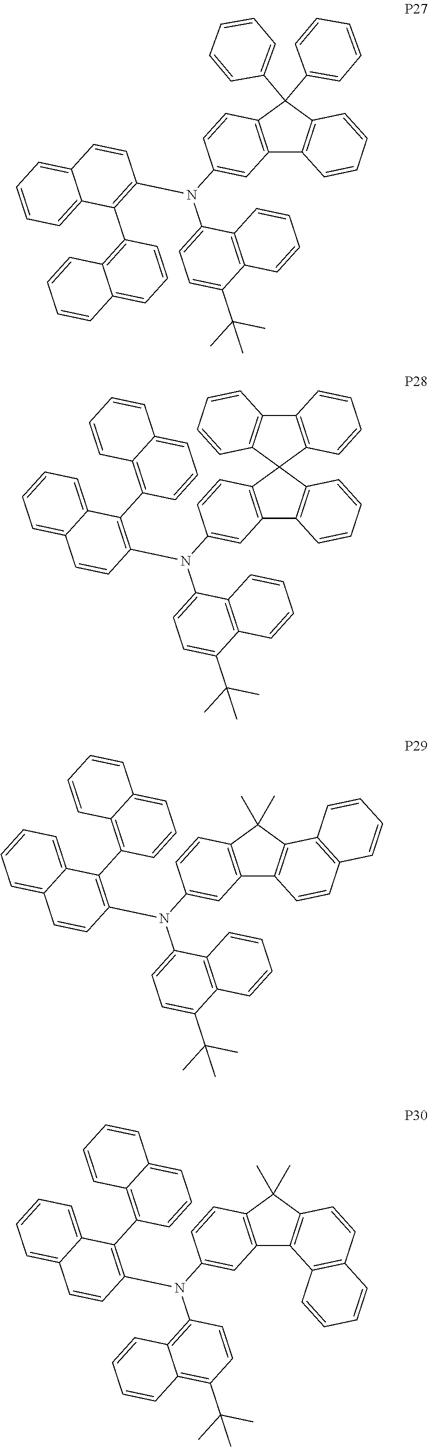

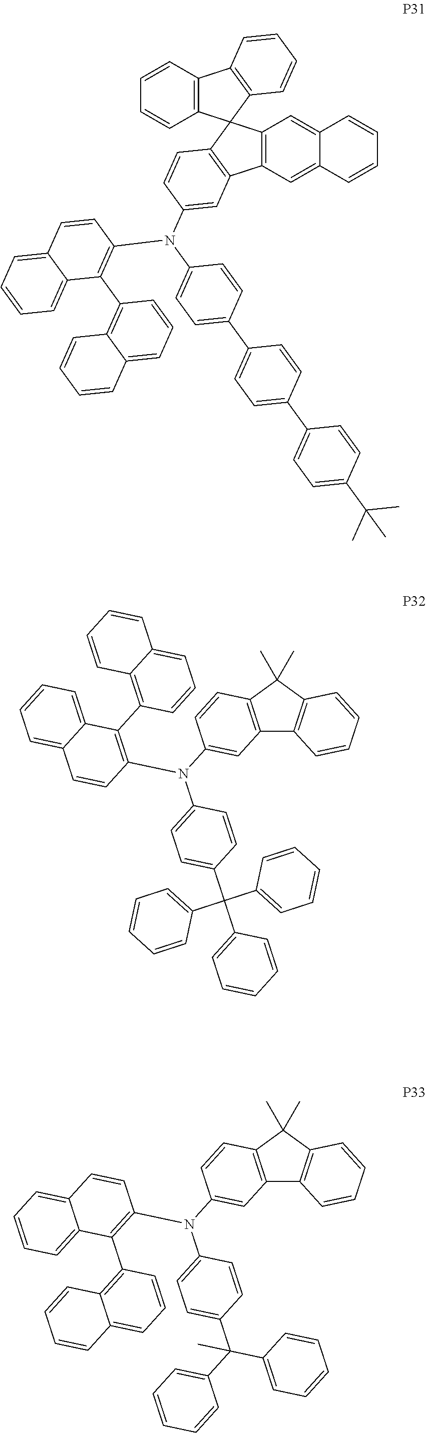 [SMILES: CC(C)(C)c1ccc(-c2ccc(-c3ccc(N(c4ccc5c(c4)-c4cc6ccccc6cc4C54c5ccccc5-c5ccccc54)c4ccc5ccccc5c4-c4cccc5ccccc45)cc3)cc2)cc1 ; CC1(C)c2ccccc2-c2cc(N(c3ccc(C(c4ccccc4)(c4ccccc4)c4ccccc4)cc3)c3ccc4ccccc4c3-c3cccc4ccccc34)ccc21 ; CC1(C)c2ccccc2-c2cc(N(c3ccc(C(C)(c4ccccc4)c4ccccc4)cc3)c3ccc4ccccc4c3-c3cccc4ccccc34)ccc21]

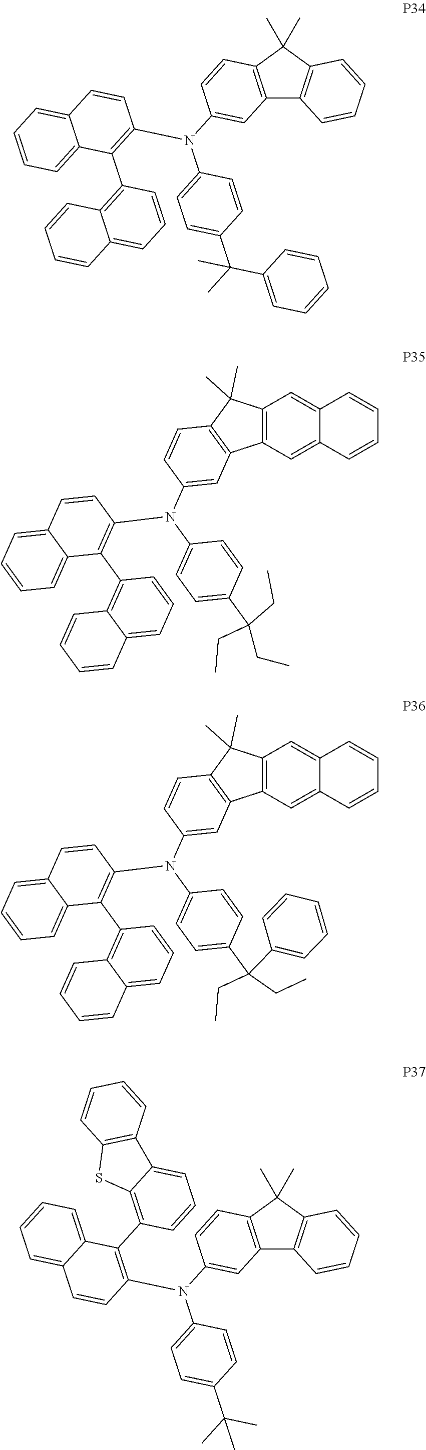 [SMILES: CC(C)(c1ccccc1)c1ccc(N(c2ccc3c(c2)-c2ccccc2C3(C)C)c2ccc3ccccc3c2-c2cccc3ccccc23)cc1 ; CCC(CC)(CC)c1ccc(N(c2ccc3c(c2)-c2cc4ccccc4cc2C3(C)C)c2ccc3ccccc3c2-c2cccc3ccccc23)cc1 ; CCC(CC)(c1ccccc1)c1ccc(N(c2ccc3c(c2)-c2cc4ccccc4cc2C3(C)C)c2ccc3ccccc3c2-c2cccc3ccccc23)cc1 ; CC(C)(C)c1ccc(N(c2ccc3c(c2)-c2ccccc2C3(C)C)c2ccc3ccccc3c2-c2cccc3c2sc2ccccc23)cc1]

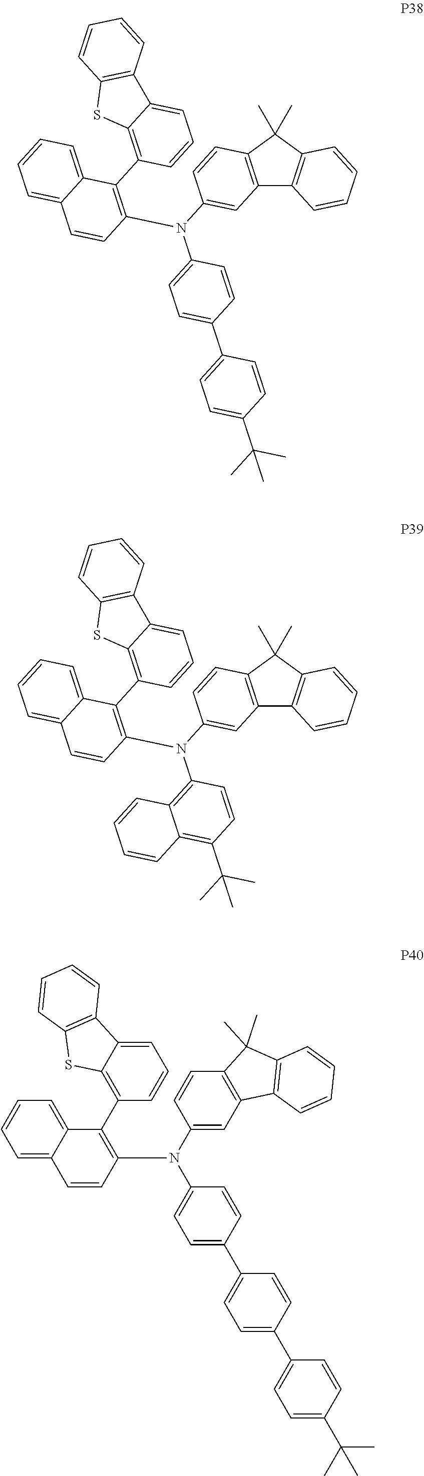 [SMILES: CC(C)(C)c1ccc(-c2ccc(N(c3ccc4c(c3)-c3ccccc3C4(C)C)c3ccc4ccccc4c3-c3cccc4c3sc3ccccc34)cc2)cc1 ; CC(C)(C)c1ccc(N(c2ccc3c(c2)-c2ccccc2C3(C)C)c2ccc3ccccc3c2-c2cccc3c2sc2ccccc23)c2ccccc12 ; CC(C)(C)c1ccc(-c2ccc(-c3ccc(N(c4ccc5c(c4)-c4ccccc4C5(C)C)c4ccc5ccccc5c4-c4cccc5c4sc4ccccc45)cc3)cc2)cc1]

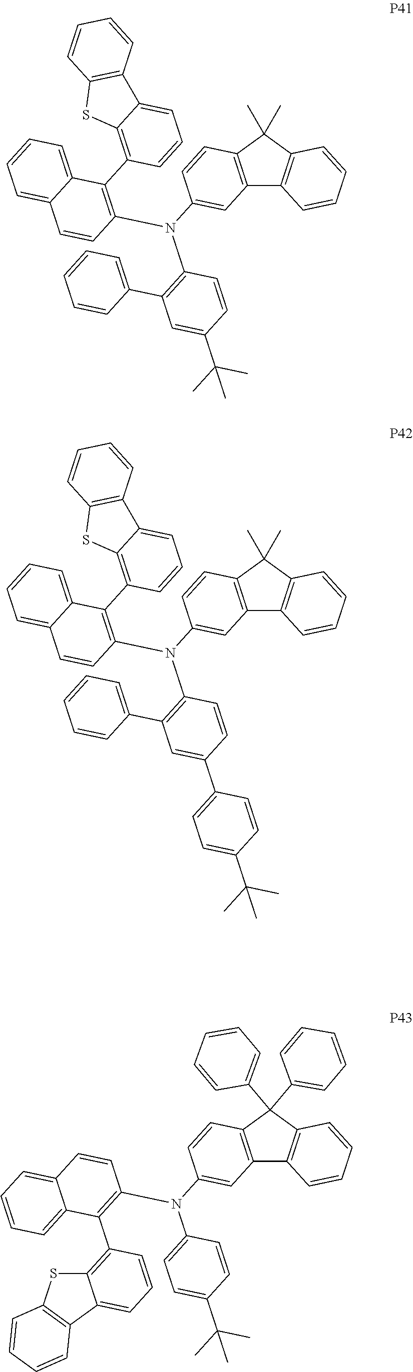 [SMILES: CC(C)(C)c1ccc(N(c2ccc3c(c2)-c2ccccc2C3(C)C)c2ccc3ccccc3c2-c2cccc3c2sc2ccccc23)c(-c2ccccc2)c1 ; CC(C)(C)c1ccc(-c2ccc(N(c3ccc4c(c3)-c3ccccc3C4(C)C)c3ccc4ccccc4c3-c3cccc4c3sc3ccccc34)c(-c3ccccc3)c2)cc1 ; CC(C)(C)c1ccc(N(c2ccc3c(c2)-c2ccccc2C3(c2ccccc2)c2ccccc2)c2ccc3ccccc3c2-c2cccc3c2sc2ccccc23)cc1]

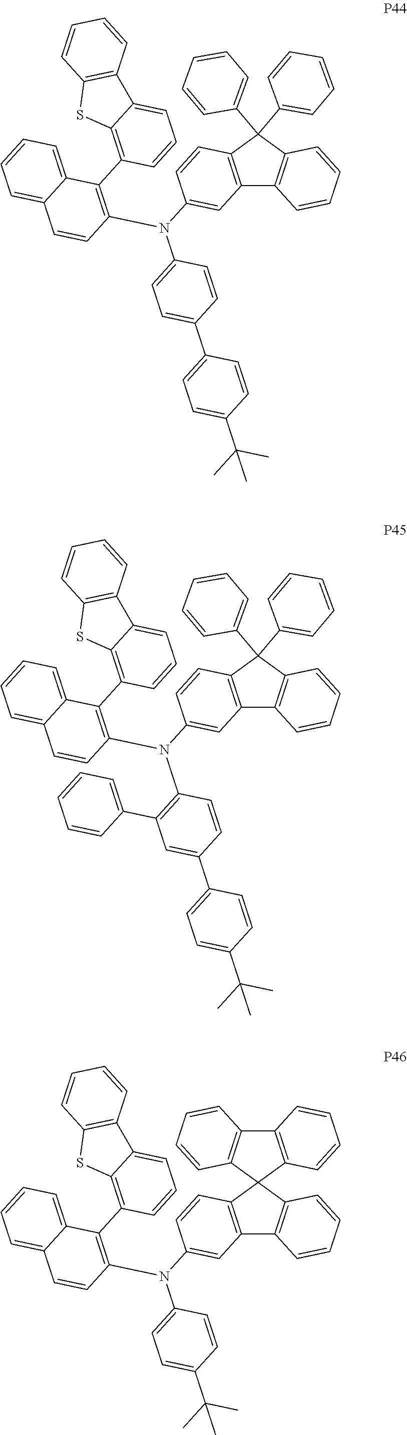 [SMILES: CC(C)(C)c1ccc(-c2ccc(N(c3ccc4c(c3)-c3ccccc3C4(c3ccccc3)c3ccccc3)c3ccc4ccccc4c3-c3cccc4c3sc3ccccc34)cc2)cc1 ; CC(C)(C)c1ccc(-c2ccc(N(c3ccc4c(c3)-c3ccccc3C4(c3ccccc3)c3ccccc3)c3ccc4ccccc4c3-c3cccc4c3sc3ccccc34)c(-c3ccccc3)c2)cc1 ; CC(C)(C)c1ccc(N(c2ccc3c(c2)-c2ccccc2C32c3ccccc3-c3ccccc32)c2ccc3ccccc3c2-c2cccc3c2sc2ccccc23)cc1]

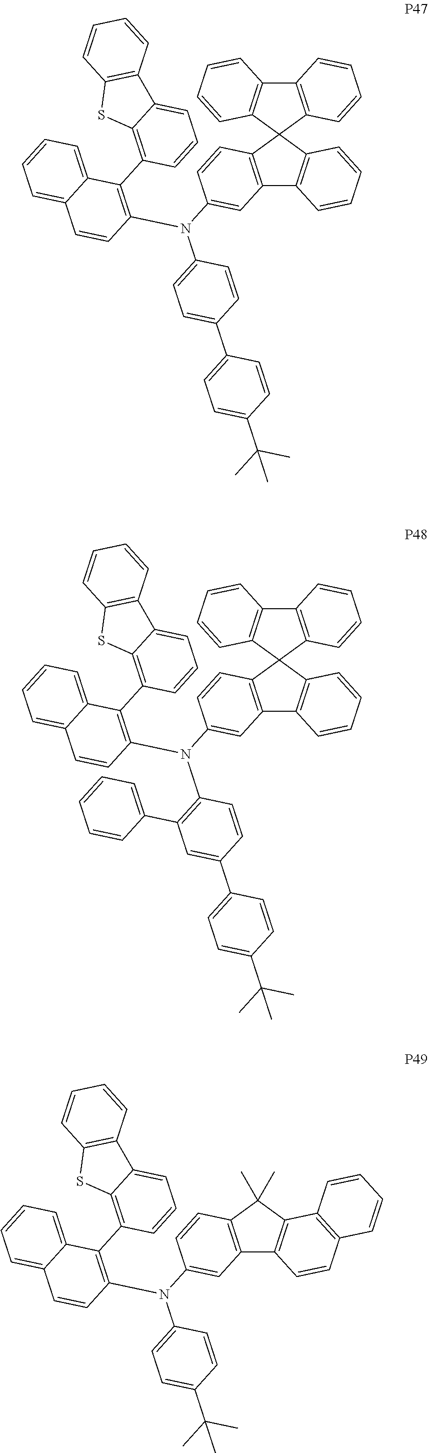 [SMILES: CC(C)(C)c1ccc(-c2ccc(N(c3ccc4c(c3)-c3ccccc3C43c4ccccc4-c4ccccc43)c3ccc4ccccc4c3-c3cccc4c3sc3ccccc34)cc2)cc1 ; CC(C)(C)c1ccc(-c2ccc(N(c3ccc4c(c3)-c3ccccc3C43c4ccccc4-c4ccccc43)c3ccc4ccccc4c3-c3cccc4c3sc3ccccc34)c(-c3ccccc3)c2)cc1 ; CC(C)(C)c1ccc(N(c2ccc3c(c2)-c2ccc4ccccc4c2C3(C)C)c2ccc3ccccc3c2-c2cccc3c2sc2ccccc23)cc1]

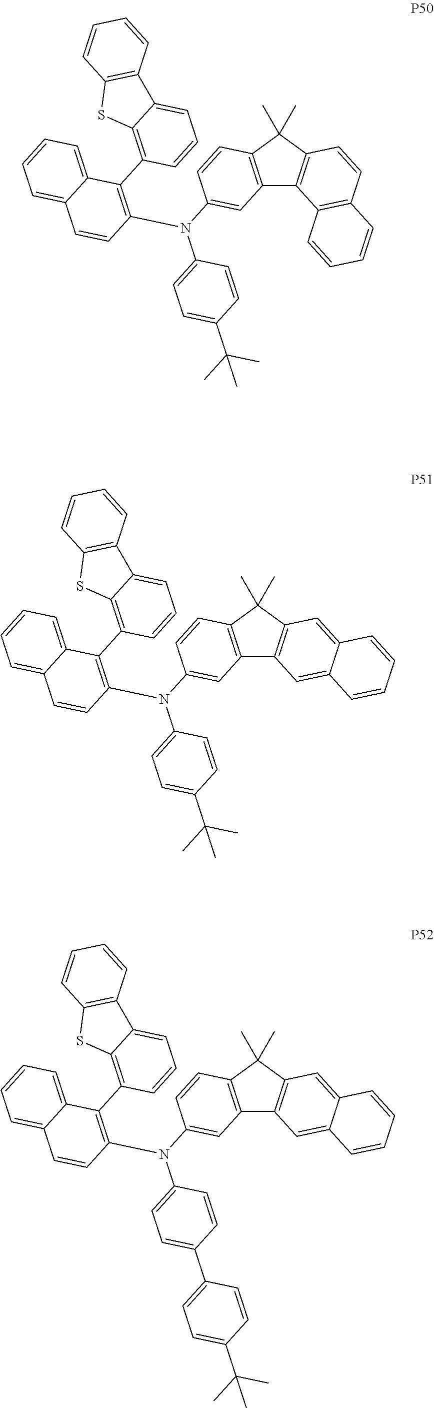 [SMILES: CC(C)(C)c1ccc(N(c2ccc3c(c2)-c2c(ccc4ccccc24)C3(C)C)c2ccc3ccccc3c2-c2cccc3c2sc2ccccc23)cc1 ; CC(C)(C)c1ccc(N(c2ccc3c(c2)-c2cc4ccccc4cc2C3(C)C)c2ccc3ccccc3c2-c2cccc3c2sc2ccccc23)cc1 ; CC(C)(C)c1ccc(-c2ccc(N(c3ccc4c(c3)-c3cc5ccccc5cc3C4(C)C)c3ccc4ccccc4c3-c3cccc4c3sc3ccccc34)cc2)cc1]

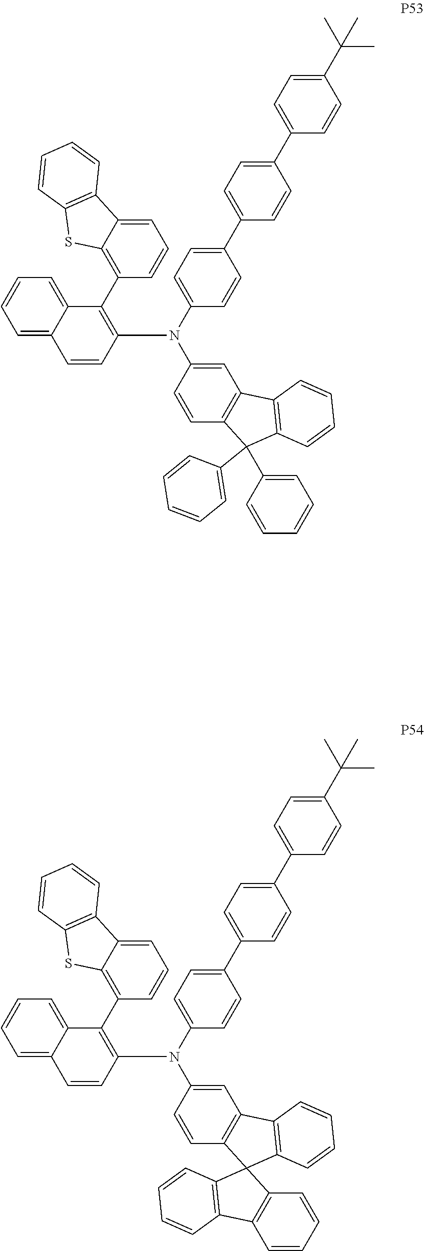 [SMILES: CC(C)(C)c1ccc(-c2ccc(-c3ccc(N(c4ccc5c(c4)-c4ccccc4C5(c4ccccc4)c4ccccc4)c4ccc5ccccc5c4-c4cccc5c4sc4ccccc45)cc3)cc2)cc1 ; CC(C)(C)c1ccc(-c2ccc(-c3ccc(N(c4ccc5c(c4)-c4ccccc4C54c5ccccc5-c5ccccc54)c4ccc5ccccc5c4-c4cccc5c4sc4ccccc45)cc3)cc2)cc1]

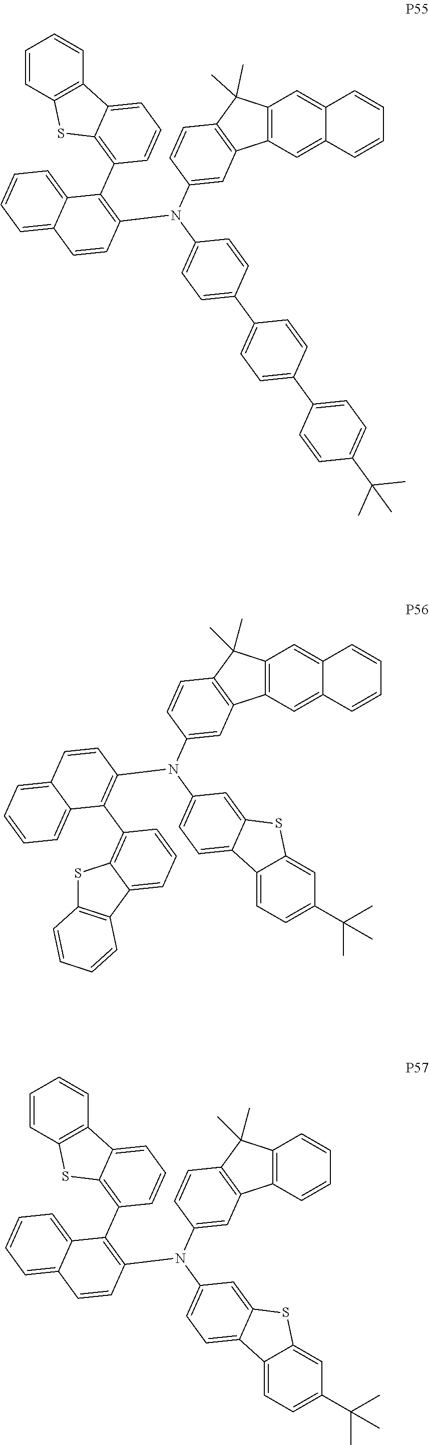 [SMILES: CC(C)(C)c1ccc(-c2ccc(-c3ccc(N(c4ccc5c(c4)-c4cc6ccccc6cc4C5(C)C)c4ccc5ccccc5c4-c4cccc5c4sc4ccccc45)cc3)cc2)cc1 ; CC(C)(C)c1ccc2c(c1)sc1cc(N(c3ccc4c(c3)-c3cc5ccccc5cc3C4(C)C)c3ccc4ccccc4c3-c3cccc4c3sc3ccccc34)ccc12 ; CC(C)(C)c1ccc2c(c1)sc1cc(N(c3ccc4c(c3)-c3ccccc3C4(C)C)c3ccc4ccccc4c3-c3cccc4c3sc3ccccc34)ccc12]

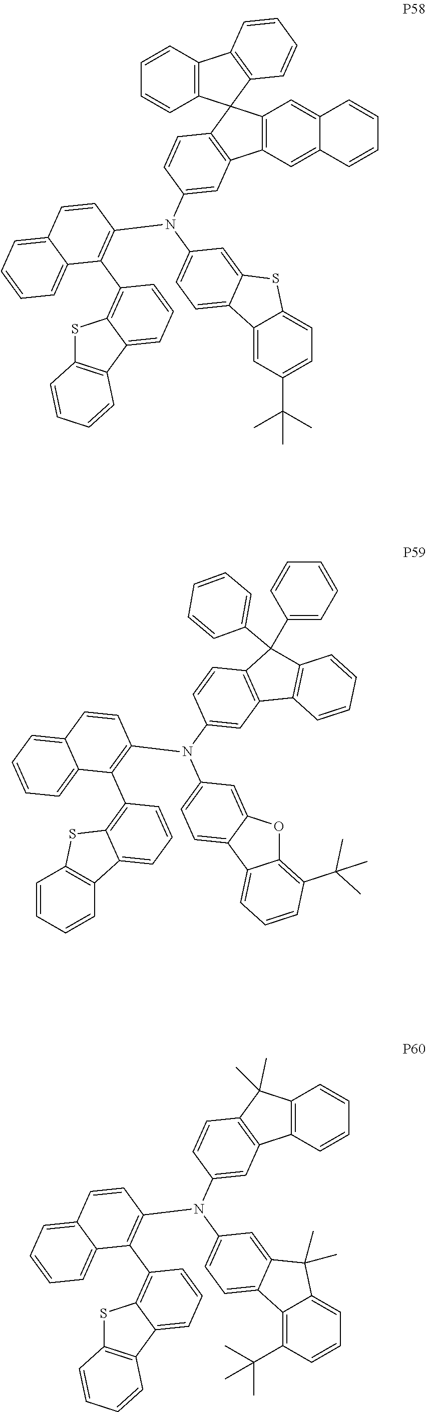 [SMILES: CC(C)(C)c1ccc2sc3cc(N(c4ccc5c(c4)-c4cc6ccccc6cc4C54c5ccccc5-c5ccccc54)c4ccc5ccccc5c4-c4cccc5c4sc4ccccc45)ccc3c2c1 ; CC(C)(C)c1cccc2c1oc1cc(N(c3ccc4c(c3)-c3ccccc3C4(c3ccccc3)c3ccccc3)c3ccc4ccccc4c3-c3cccc4c3sc3ccccc34)ccc12 ; CC(C)(C)c1cccc2c1-c1ccc(N(c3ccc4c(c3)-c3ccccc3C4(C)C)c3ccc4ccccc4c3-c3cccc4c3sc3ccccc34)cc1C2(C)C]

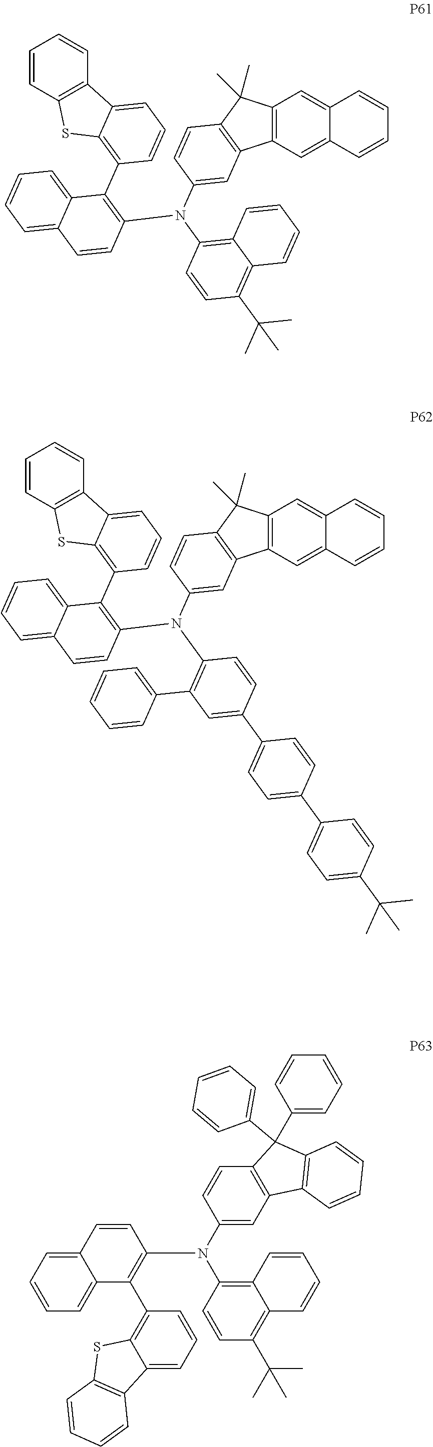 [SMILES: CC(C)(C)c1ccc(N(c2ccc3c(c2)-c2cc4ccccc4cc2C3(C)C)c2ccc3ccccc3c2-c2cccc3c2sc2ccccc23)c2ccccc12 ; CC(C)(C)c1ccc(-c2ccc(-c3ccc(N(c4ccc5c(c4)-c4cc6ccccc6cc4C5(C)C)c4ccc5ccccc5c4-c4cccc5c4sc4ccccc45)c(-c4ccccc4)c3)cc2)cc1 ; CC(C)(C)c1ccc(N(c2ccc3c(c2)-c2ccccc2C3(c2ccccc2)c2ccccc2)c2ccc3ccccc3c2-c2cccc3c2sc2ccccc23)c2ccccc12]

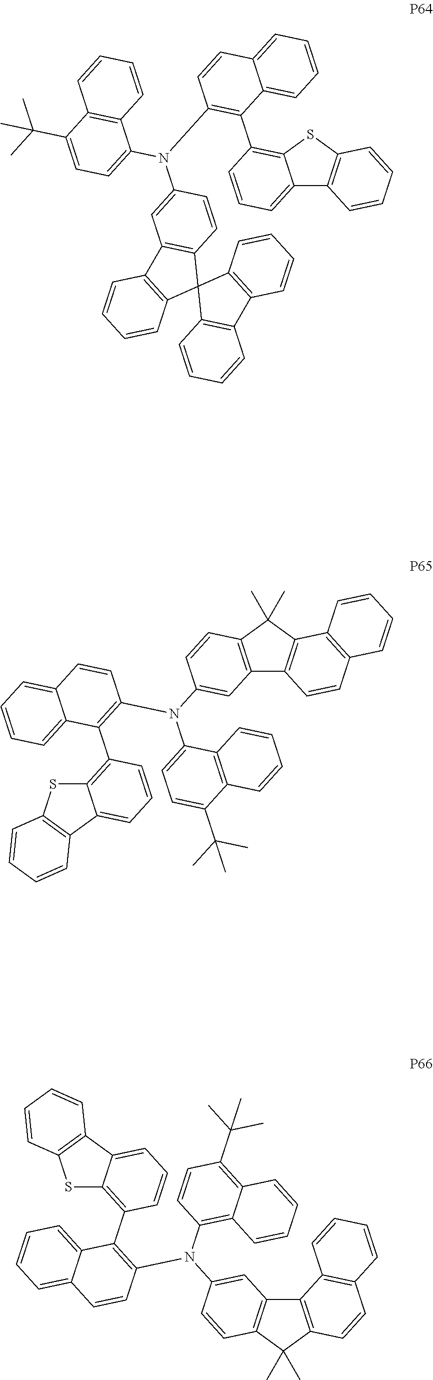 [SMILES: CC(C)(C)c1ccc(N(c2ccc3c(c2)-c2ccccc2C32c3ccccc3-c3ccccc32)c2ccc3ccccc3c2-c2cccc3c2sc2ccccc23)c2ccccc12 ; CC(C)(C)c1ccc(N(c2ccc3c(c2)-c2ccc4ccccc4c2C3(C)C)c2ccc3ccccc3c2-c2cccc3c2sc2ccccc23)c2ccccc12 ; CC(C)(C)c1ccc(N(c2ccc3c(c2)-c2c(ccc4ccccc24)C3(C)C)c2ccc3ccccc3c2-c2cccc3c2sc2ccccc23)c2ccccc12]

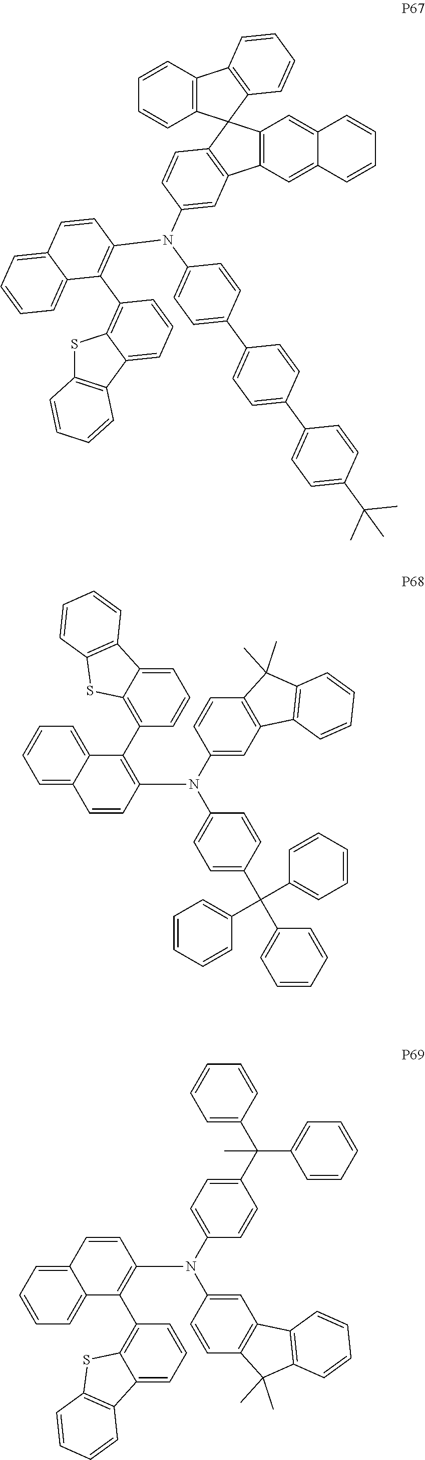 [SMILES: CC(C)(C)c1ccc(-c2ccc(-c3ccc(N(c4ccc5c(c4)-c4cc6ccccc6cc4C54c5ccccc5-c5ccccc54)c4ccc5ccccc5c4-c4cccc5c4sc4ccccc45)cc3)cc2)cc1 ; CC1(C)c2ccccc2-c2cc(N(c3ccc(C(c4ccccc4)(c4ccccc4)c4ccccc4)cc3)c3ccc4ccccc4c3-c3cccc4c3sc3ccccc34)ccc21 ; CC1(C)c2ccccc2-c2cc(N(c3ccc(C(C)(c4ccccc4)c4ccccc4)cc3)c3ccc4ccccc4c3-c3cccc4c3sc3ccccc34)ccc21]

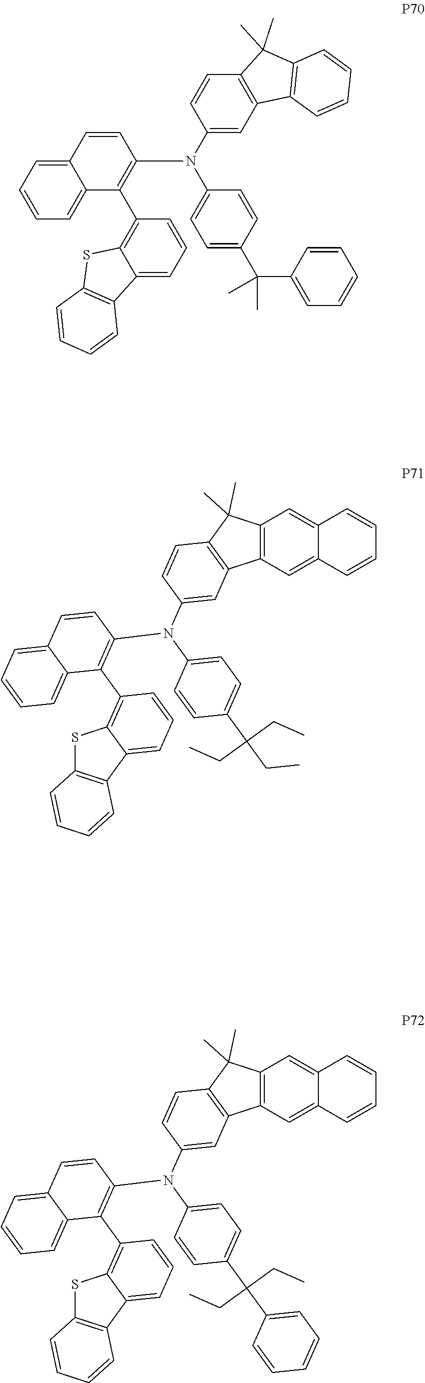 [SMILES: CC(C)(c1ccccc1)c1ccc(N(c2ccc3c(c2)-c2ccccc2C3(C)C)c2ccc3ccccc3c2-c2cccc3c2sc2ccccc23)cc1 ; CCC(CC)(CC)c1ccc(N(c2ccc3c(c2)-c2cc4ccccc4cc2C3(C)C)c2ccc3ccccc3c2-c2cccc3c2sc2ccccc23)cc1 ; CCC(CC)(c1ccccc1)c1ccc(N(c2ccc3c(c2)-c2cc4ccccc4cc2C3(C)C)c2ccc3ccccc3c2-c2cccc3c2sc2ccccc23)cc1]

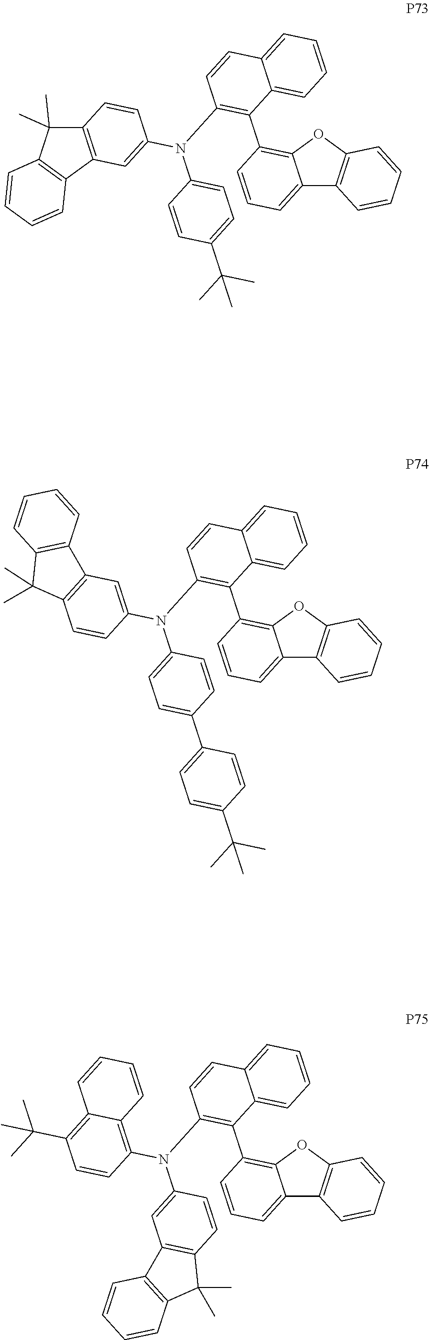 [SMILES: CC(C)(C)c1ccc(N(c2ccc3c(c2)-c2ccccc2C3(C)C)c2ccc3ccccc3c2-c2cccc3c2oc2ccccc23)cc1 ; CC(C)(C)c1ccc(-c2ccc(N(c3ccc4c(c3)-c3ccccc3C4(C)C)c3ccc4ccccc4c3-c3cccc4c3oc3ccccc34)cc2)cc1 ; CC(C)(C)c1ccc(N(c2ccc3c(c2)-c2ccccc2C3(C)C)c2ccc3ccccc3c2-c2cccc3c2oc2ccccc23)c2ccccc12]

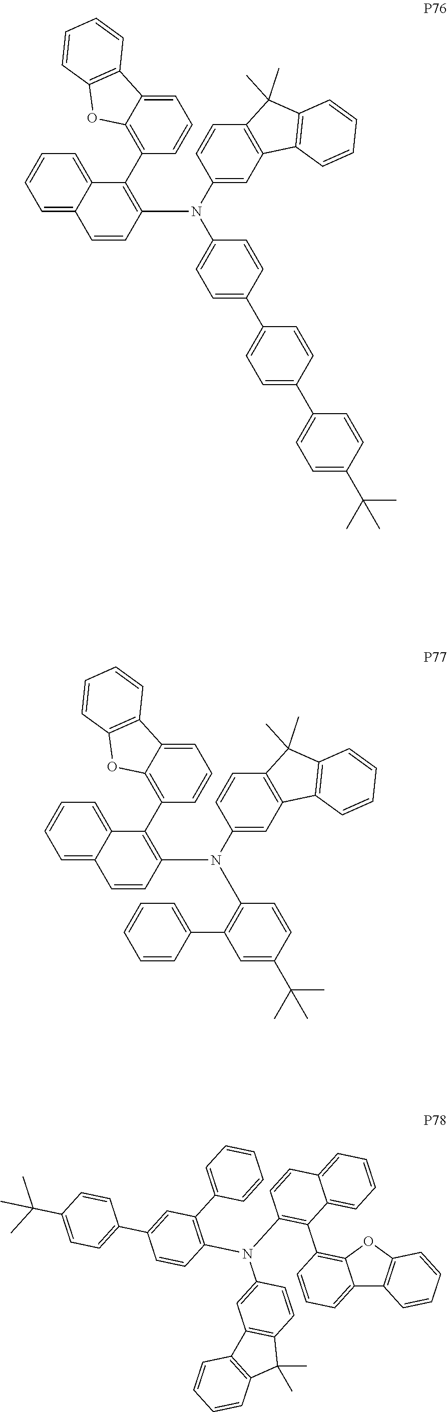

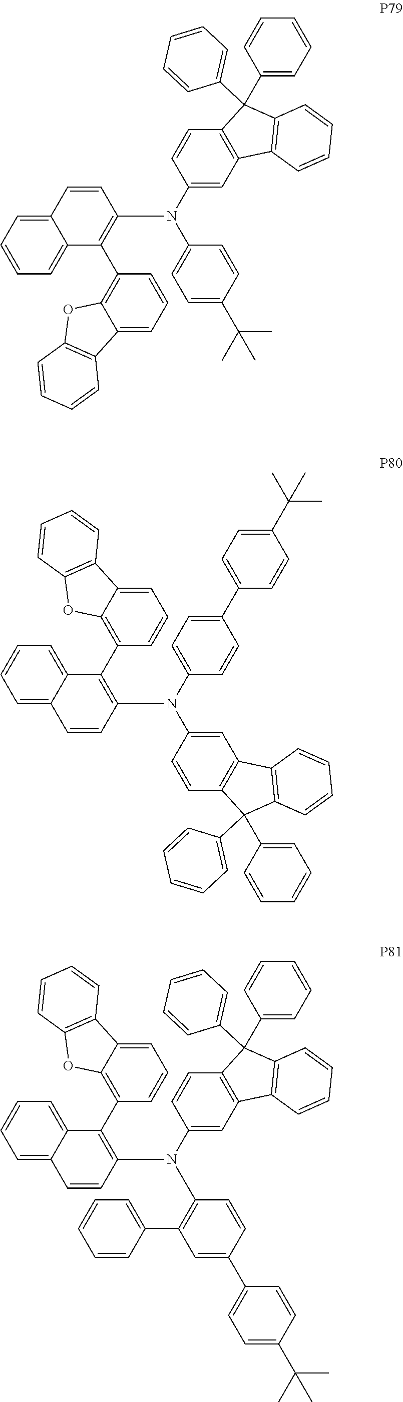 [SMILES: CC(C)(C)c1ccc(N(c2ccc3c(c2)-c2ccccc2C3(c2ccccc2)c2ccccc2)c2ccc3ccccc3c2-c2cccc3c2oc2ccccc23)cc1 ; CC(C)(C)c1ccc(-c2ccc(N(c3ccc4c(c3)-c3ccccc3C4(c3ccccc3)c3ccccc3)c3ccc4ccccc4c3-c3cccc4c3oc3ccccc34)cc2)cc1 ; CC(C)(C)c1ccc(-c2ccc(N(c3ccc4c(c3)-c3ccccc3C4(c3ccccc3)c3ccccc3)c3ccc4ccccc4c3-c3cccc4c3oc3ccccc34)c(-c3ccccc3)c2)cc1]

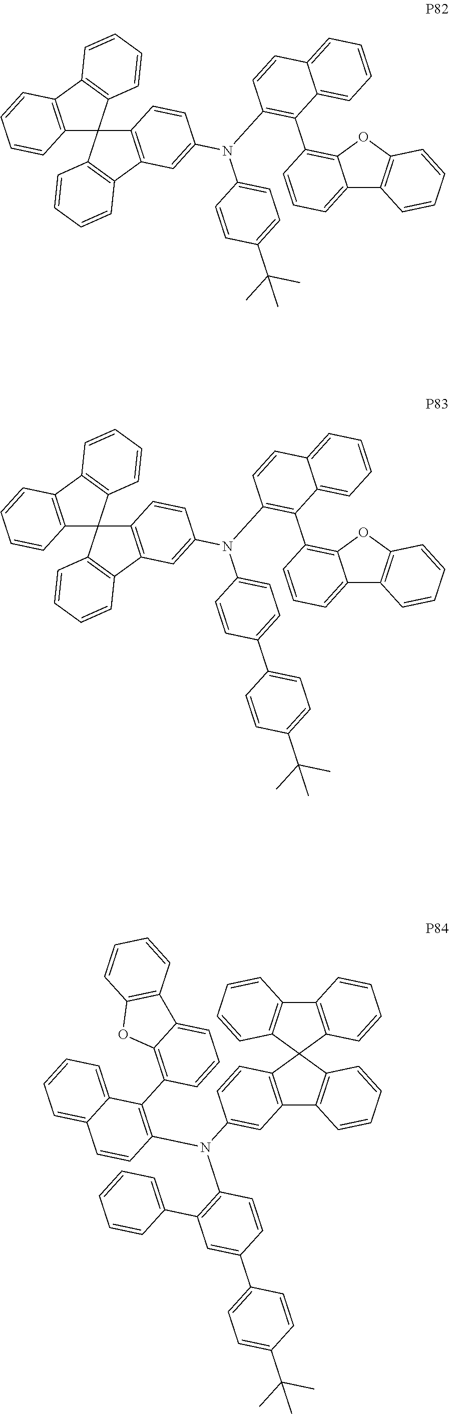 [SMILES: CC(C)(C)c1ccc(N(c2ccc3c(c2)-c2ccccc2C32c3ccccc3-c3ccccc32)c2ccc3ccccc3c2-c2cccc3c2oc2ccccc23)cc1 ; CC(C)(C)c1ccc(-c2ccc(N(c3ccc4c(c3)-c3ccccc3C43c4ccccc4-c4ccccc43)c3ccc4ccccc4c3-c3cccc4c3oc3ccccc34)cc2)cc1 ; CC(C)(C)c1ccc(-c2ccc(N(c3ccc4c(c3)-c3ccccc3C43c4ccccc4-c4ccccc43)c3ccc4ccccc4c3-c3cccc4c3oc3ccccc34)c(-c3ccccc3)c2)cc1]

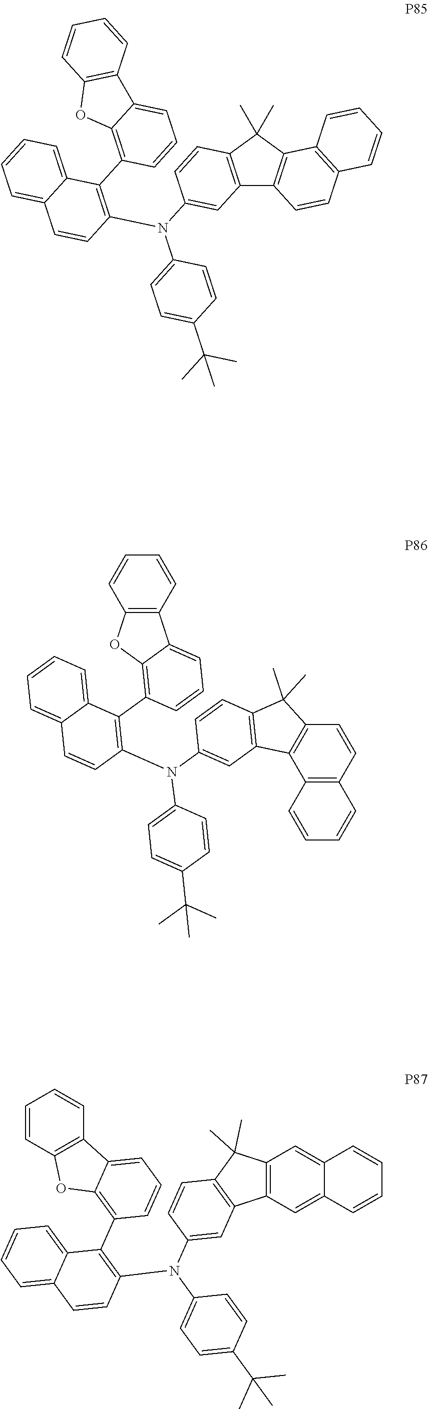 [SMILES: CC(C)(C)c1ccc(N(c2ccc3c(c2)-c2ccc4ccccc4c2C3(C)C)c2ccc3ccccc3c2-c2cccc3c2oc2ccccc23)cc1 ; CC(C)(C)c1ccc(N(c2ccc3c(c2)-c2c(ccc4ccccc24)C3(C)C)c2ccc3ccccc3c2-c2cccc3c2oc2ccccc23)cc1 ; CC(C)(C)c1ccc(N(c2ccc3c(c2)-c2cc4ccccc4cc2C3(C)C)c2ccc3ccccc3c2-c2cccc3c2oc2ccccc23)cc1]

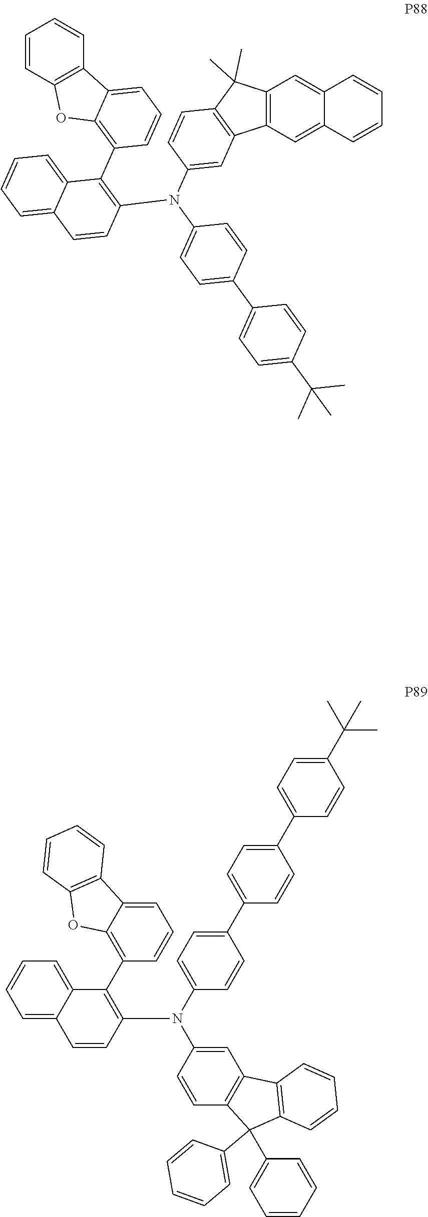 [SMILES: CC(C)(C)c1ccc(-c2ccc(N(c3ccc4c(c3)-c3cc5ccccc5cc3C4(C)C)c3ccc4ccccc4c3-c3cccc4c3oc3ccccc34)cc2)cc1 ; CC(C)(C)c1ccc(-c2ccc(-c3ccc(N(c4ccc5c(c4)-c4ccccc4C5(c4ccccc4)c4ccccc4)c4ccc5ccccc5c4-c4cccc5c4oc4ccccc45)cc3)cc2)cc1]

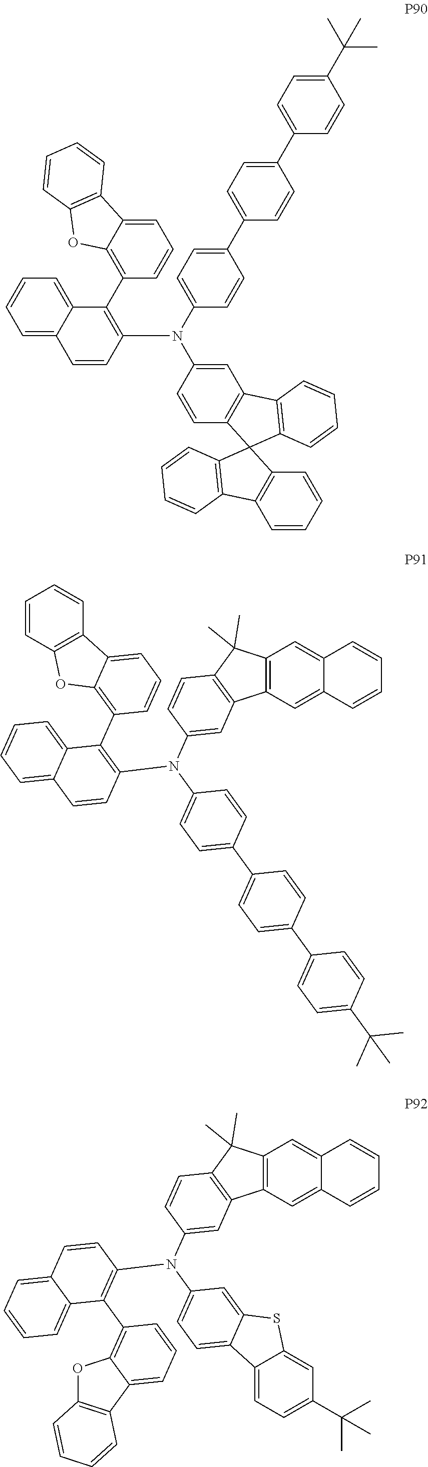 [SMILES: CC(C)(C)c1ccc(-c2ccc(-c3ccc(N(c4ccc5c(c4)-c4ccccc4C54c5ccccc5-c5ccccc54)c4ccc5ccccc5c4-c4cccc5c4oc4ccccc45)cc3)cc2)cc1 ; CC(C)(C)c1ccc(-c2ccc(-c3ccc(N(c4ccc5c(c4)-c4cc6ccccc6cc4C5(C)C)c4ccc5ccccc5c4-c4cccc5c4oc4ccccc45)cc3)cc2)cc1 ; CC(C)(C)c1ccc2c(c1)sc1cc(N(c3ccc4c(c3)-c3cc5ccccc5cc3C4(C)C)c3ccc4ccccc4c3-c3cccc4c3oc3ccccc34)ccc12]

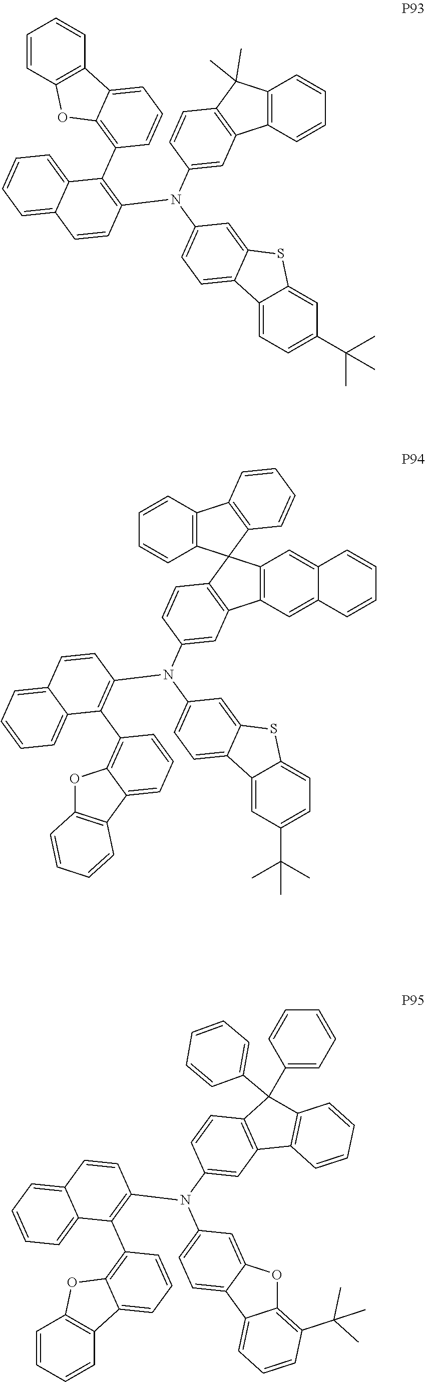 [SMILES: CC(C)(C)c1ccc2c(c1)sc1cc(N(c3ccc4c(c3)-c3ccccc3C4(C)C)c3ccc4ccccc4c3-c3cccc4c3oc3ccccc34)ccc12 ; CC(C)(C)c1ccc2sc3cc(N(c4ccc5c(c4)-c4cc6ccccc6cc4C54c5ccccc5-c5ccccc54)c4ccc5ccccc5c4-c4cccc5c4oc4ccccc45)ccc3c2c1 ; CC(C)(C)c1cccc2c1oc1cc(N(c3ccc4c(c3)-c3ccccc3C4(c3ccccc3)c3ccccc3)c3ccc4ccccc4c3-c3cccc4c3oc3ccccc34)ccc12]

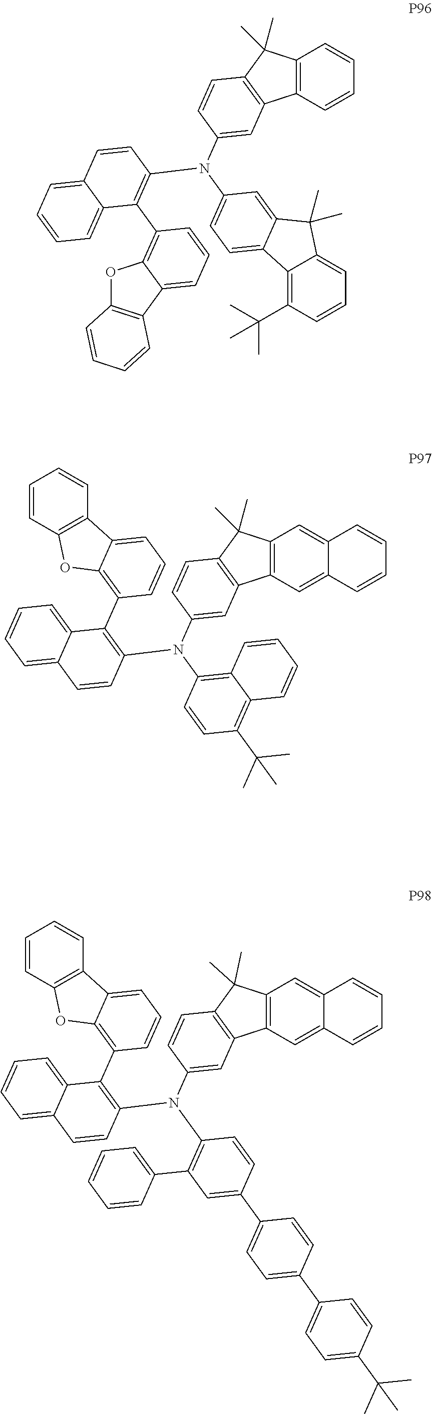 [SMILES: CC(C)(C)c1cccc2c1-c1ccc(N(c3ccc4c(c3)-c3ccccc3C4(C)C)c3ccc4ccccc4c3-c3cccc4c3oc3ccccc34)cc1C2(C)C ; CC(C)(C)c1ccc(N(c2ccc3c(c2)-c2cc4ccccc4cc2C3(C)C)c2ccc3ccccc3c2-c2cccc3c2oc2ccccc23)c2ccccc12 ; CC(C)(C)c1ccc(-c2ccc(-c3ccc(N(c4ccc5c(c4)-c4cc6ccccc6cc4C5(C)C)c4ccc5ccccc5c4-c4cccc5c4oc4ccccc45)c(-c4ccccc4)c3)cc2)cc1]

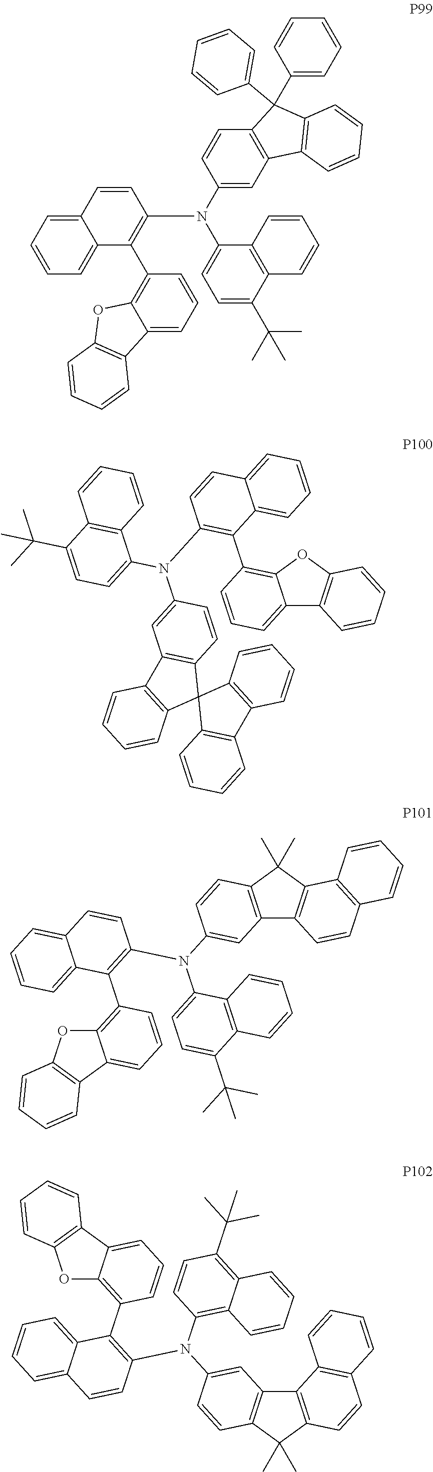 [SMILES: CC(C)(C)c1ccc(N(c2ccc3c(c2)-c2ccccc2C3(c2ccccc2)c2ccccc2)c2ccc3ccccc3c2-c2cccc3c2oc2ccccc23)c2ccccc12 ; CC(C)(C)c1ccc(N(c2ccc3c(c2)-c2ccccc2C32c3ccccc3-c3ccccc32)c2ccc3ccccc3c2-c2cccc3c2oc2ccccc23)c2ccccc12 ; CC(C)(C)c1ccc(N(c2ccc3c(c2)-c2ccc4ccccc4c2C3(C)C)c2ccc3ccccc3c2-c2cccc3c2oc2ccccc23)c2ccccc12 ; CC(C)(C)c1ccc(N(c2ccc3c(c2)-c2c(ccc4ccccc24)C3(C)C)c2ccc3ccccc3c2-c2cccc3c2oc2ccccc23)c2ccccc12]

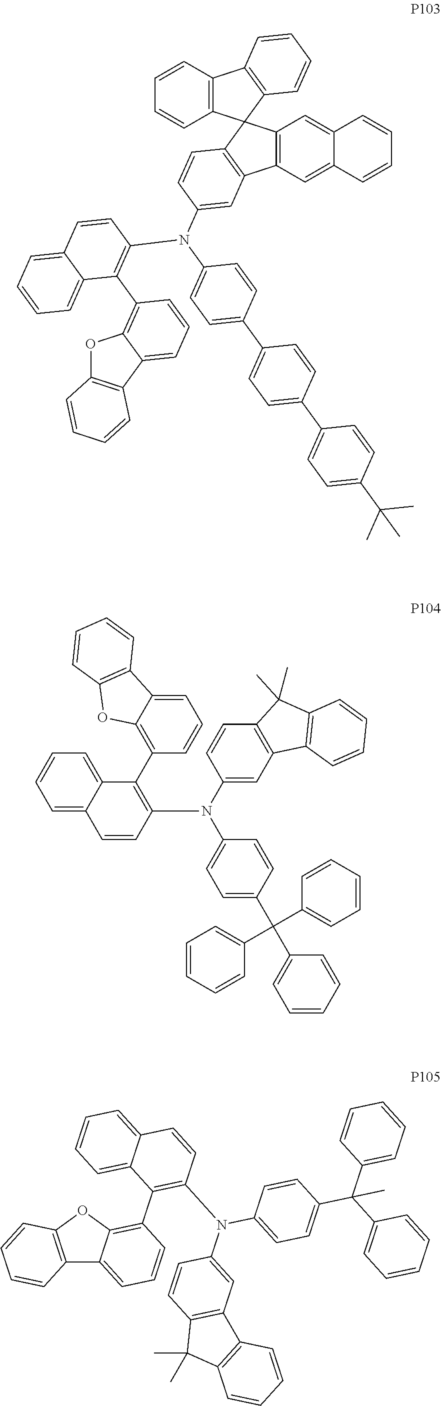 [SMILES: CC(C)(C)c1ccc(-c2ccc(-c3ccc(N(c4ccc5c(c4)-c4cc6ccccc6cc4C54c5ccccc5-c5ccccc54)c4ccc5ccccc5c4-c4cccc5c4oc4ccccc45)cc3)cc2)cc1 ; CC1(C)c2ccccc2-c2cc(N(c3ccc(C(c4ccccc4)(c4ccccc4)c4ccccc4)cc3)c3ccc4ccccc4c3-c3cccc4c3oc3ccccc34)ccc21 ; CC1(C)c2ccccc2-c2cc(N(c3ccc(C(C)(c4ccccc4)c4ccccc4)cc3)c3ccc4ccccc4c3-c3cccc4c3oc3ccccc34)ccc21]

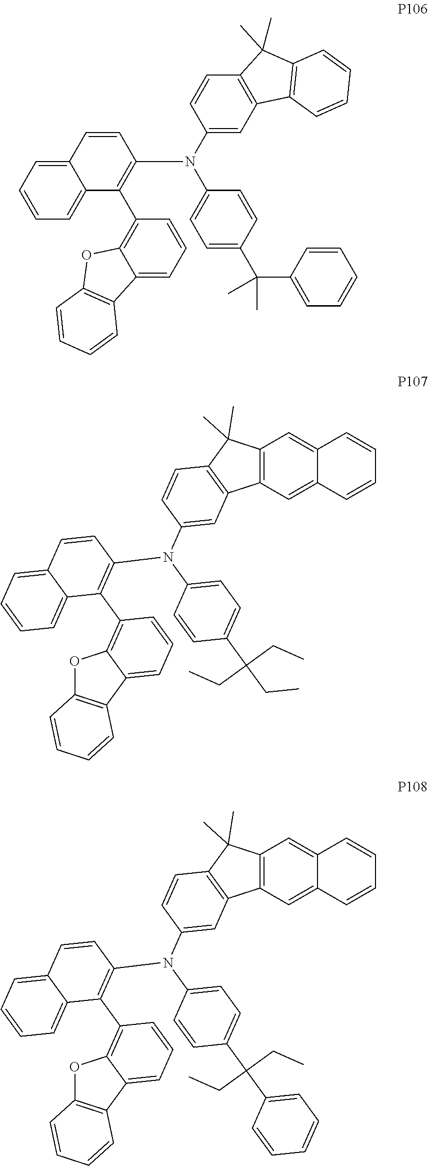 [SMILES: CC(C)(c1ccccc1)c1ccc(N(c2ccc3c(c2)-c2ccccc2C3(C)C)c2ccc3ccccc3c2-c2cccc3c2oc2ccccc23)cc1 ; CCC(CC)(CC)c1ccc(N(c2ccc3c(c2)-c2cc4ccccc4cc2C3(C)C)c2ccc3ccccc3c2-c2cccc3c2oc2ccccc23)cc1 ; CCC(CC)(c1ccccc1)c1ccc(N(c2ccc3c(c2)-c2cc4ccccc4cc2C3(C)C)c2ccc3ccccc3c2-c2cccc3c2oc2ccccc23)cc1]

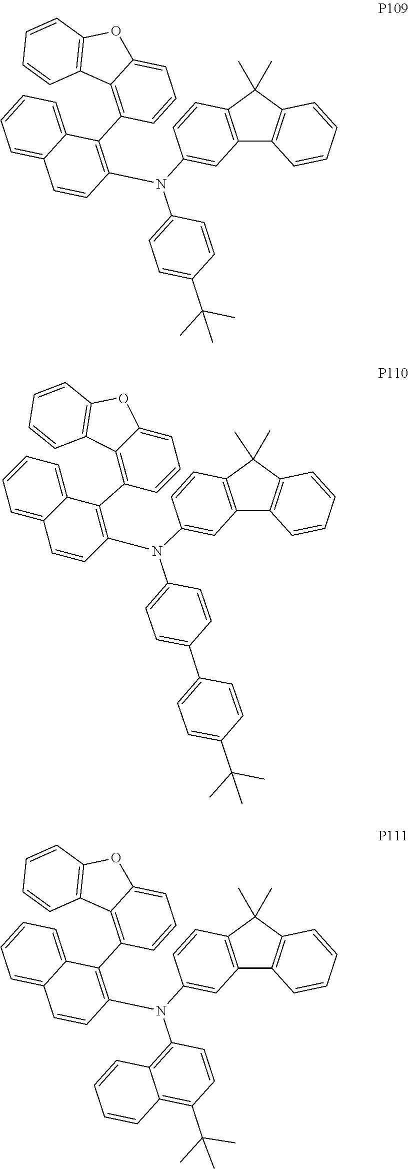 [SMILES: CC(C)(C)c1ccc(N(c2ccc3c(c2)-c2ccccc2C3(C)C)c2ccc3ccccc3c2-c2cccc3oc4ccccc4c23)cc1 ; CC(C)(C)c1ccc(-c2ccc(N(c3ccc4c(c3)-c3ccccc3C4(C)C)c3ccc4ccccc4c3-c3cccc4oc5ccccc5c34)cc2)cc1 ; CC(C)(C)c1ccc(N(c2ccc3c(c2)-c2ccccc2C3(C)C)c2ccc3ccccc3c2-c2cccc3oc4ccccc4c23)c2ccccc12]

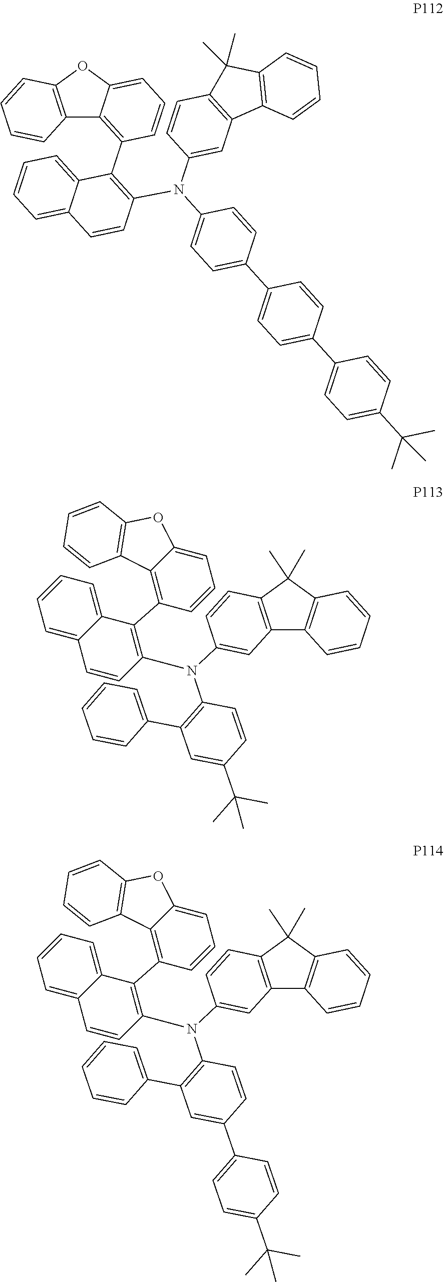 [SMILES: CC(C)(C)c1ccc(-c2ccc(-c3ccc(N(c4ccc5c(c4)-c4ccccc4C5(C)C)c4ccc5ccccc5c4-c4cccc5oc6ccccc6c45)cc3)cc2)cc1 ; CC(C)(C)c1ccc(N(c2ccc3c(c2)-c2ccccc2C3(C)C)c2ccc3ccccc3c2-c2cccc3oc4ccccc4c23)c(-c2ccccc2)c1 ; CC(C)(C)c1ccc(-c2ccc(N(c3ccc4c(c3)-c3ccccc3C4(C)C)c3ccc4ccccc4c3-c3cccc4oc5ccccc5c34)c(-c3ccccc3)c2)cc1]

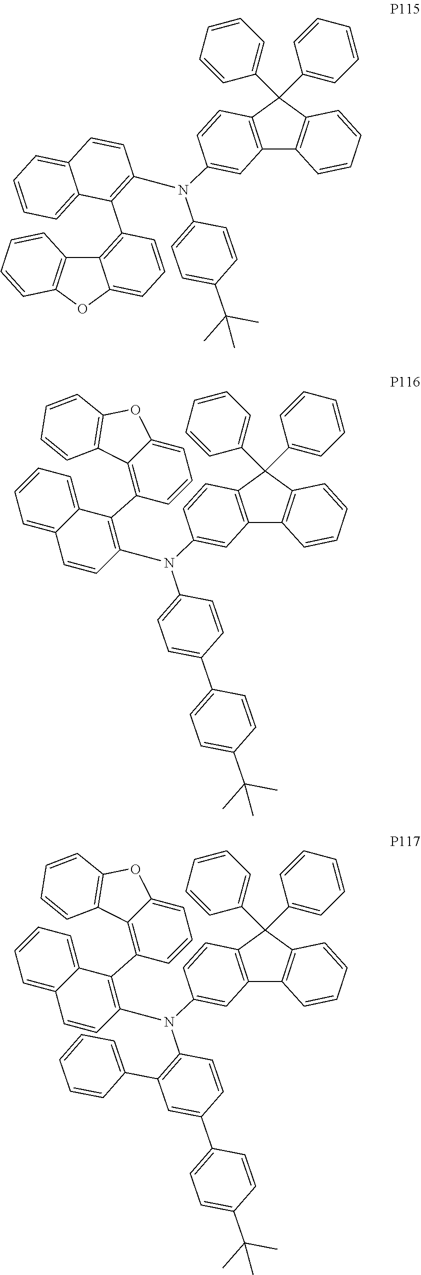

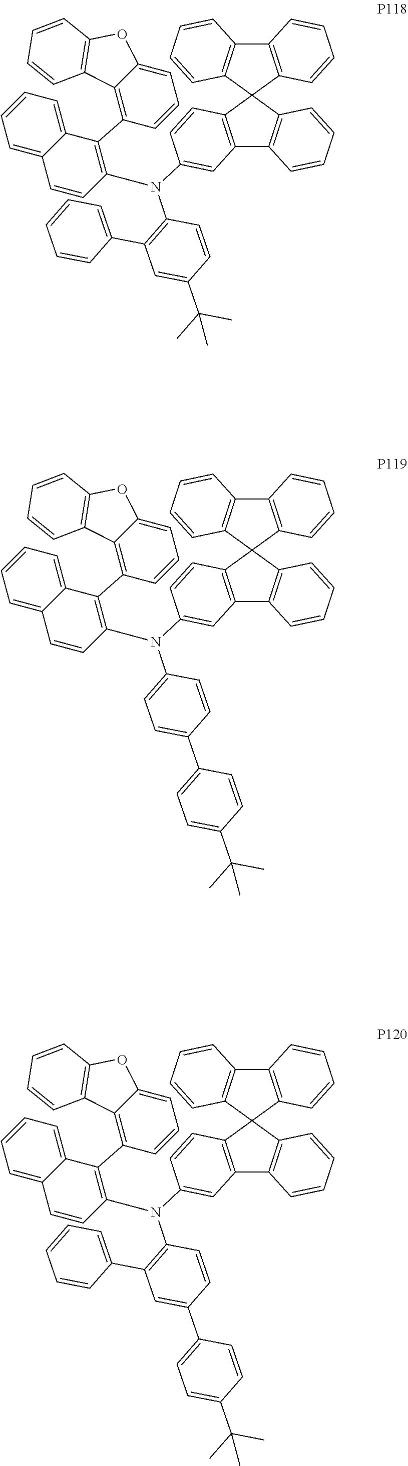 [SMILES: CC(C)(C)c1ccc(N(c2ccc3c(c2)-c2ccccc2C32c3ccccc3-c3ccccc32)c2ccc3ccccc3c2-c2cccc3oc4ccccc4c23)c(-c2ccccc2)c1 ; CC(C)(C)c1ccc(-c2ccc(N(c3ccc4c(c3)-c3ccccc3C43c4ccccc4-c4ccccc43)c3ccc4ccccc4c3-c3cccc4oc5ccccc5c34)cc2)cc1 ; CC(C)(C)c1ccc(-c2ccc(N(c3ccc4c(c3)-c3ccccc3C43c4ccccc4-c4ccccc43)c3ccc4ccccc4c3-c3cccc4oc5ccccc5c34)c(-c3ccccc3)c2)cc1]

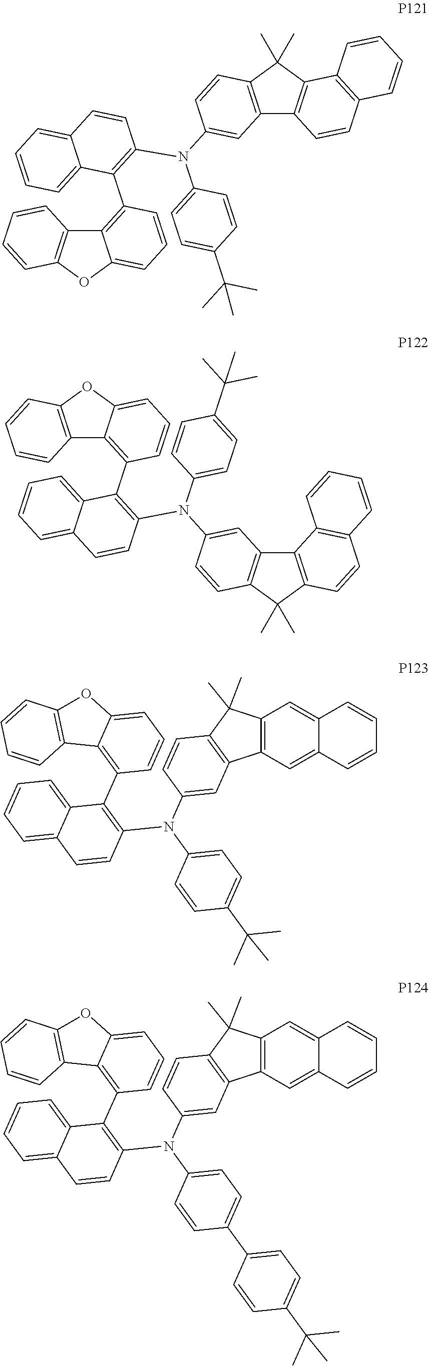

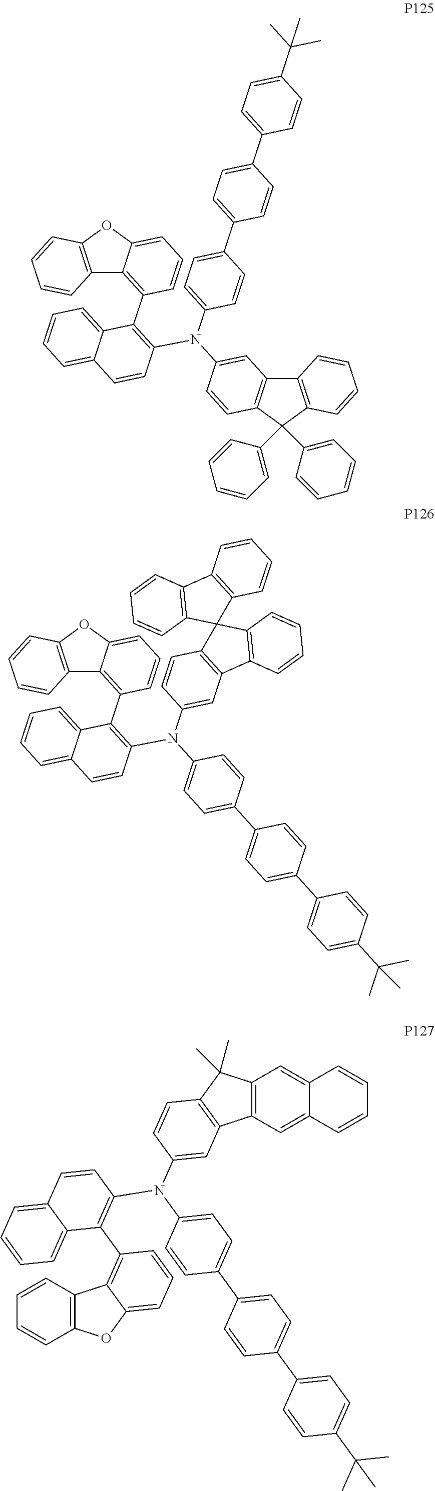

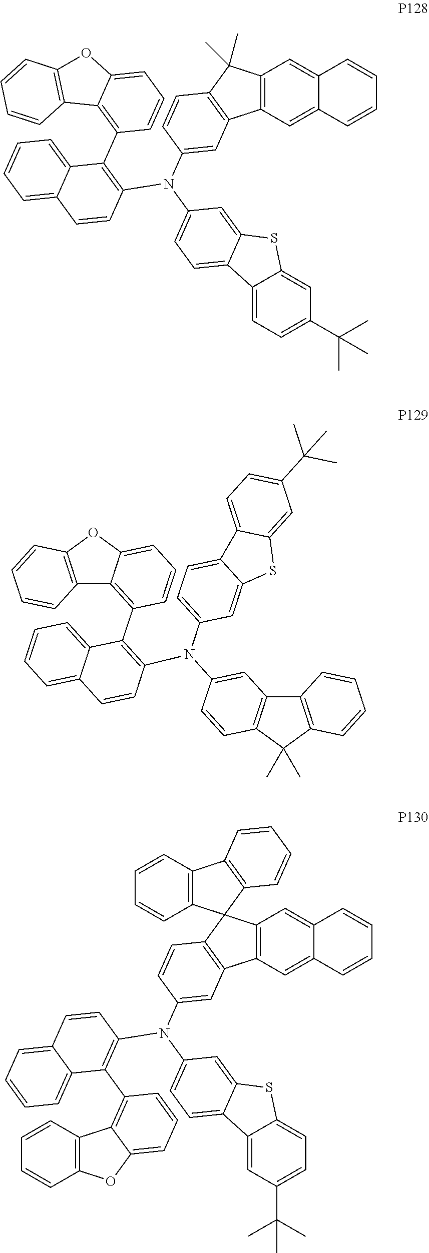 [SMILES: CC(C)(C)c1ccc2c(c1)sc1cc(N(c3ccc4c(c3)-c3cc5ccccc5cc3C4(C)C)c3ccc4ccccc4c3-c3cccc4oc5ccccc5c34)ccc12 ; CC(C)(C)c1ccc2c(c1)sc1cc(N(c3ccc4c(c3)-c3ccccc3C4(C)C)c3ccc4ccccc4c3-c3cccc4oc5ccccc5c34)ccc12 ; CC(C)(C)c1ccc2sc3cc(N(c4ccc5c(c4)-c4cc6ccccc6cc4C54c5ccccc5-c5ccccc54)c4ccc5ccccc5c4-c4cccc5oc6ccccc6c45)ccc3c2c1]

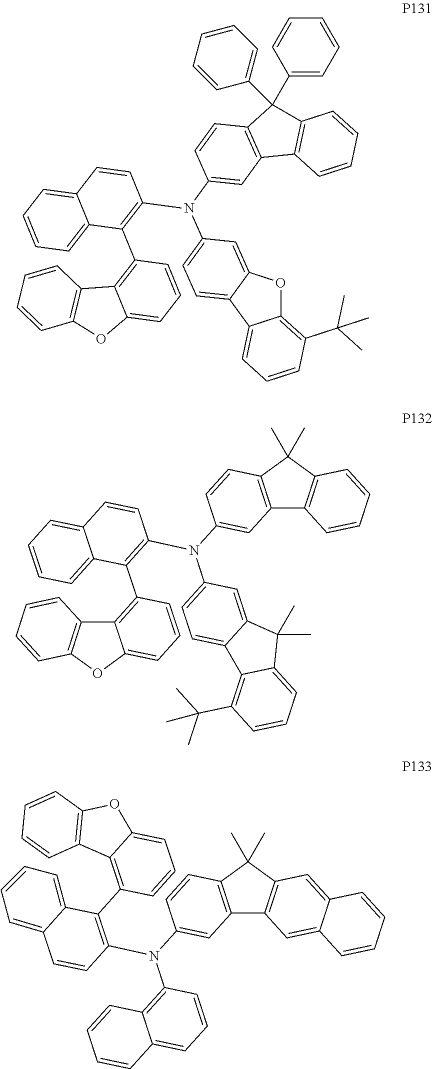 [SMILES: CC(C)(C)c1cccc2c1oc1cc(N(c3ccc4c(c3)-c3ccccc3C4(c3ccccc3)c3ccccc3)c3ccc4ccccc4c3-c3cccc4oc5ccccc5c34)ccc12 ; CC(C)(C)c1cccc2c1-c1ccc(N(c3ccc4c(c3)-c3ccccc3C4(C)C)c3ccc4ccccc4c3-c3cccc4oc5ccccc5c34)cc1C2(C)C ; CC1(C)c2ccc(N(c3ccc4ccccc4c3-c3cccc4oc5ccccc5c34)c3cccc4ccccc34)cc2-c2cc3ccccc3cc21]

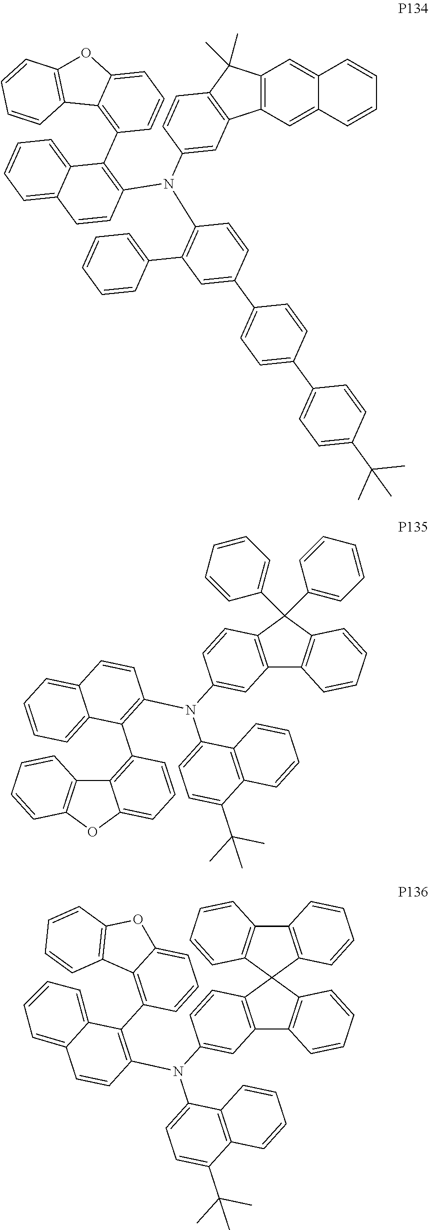

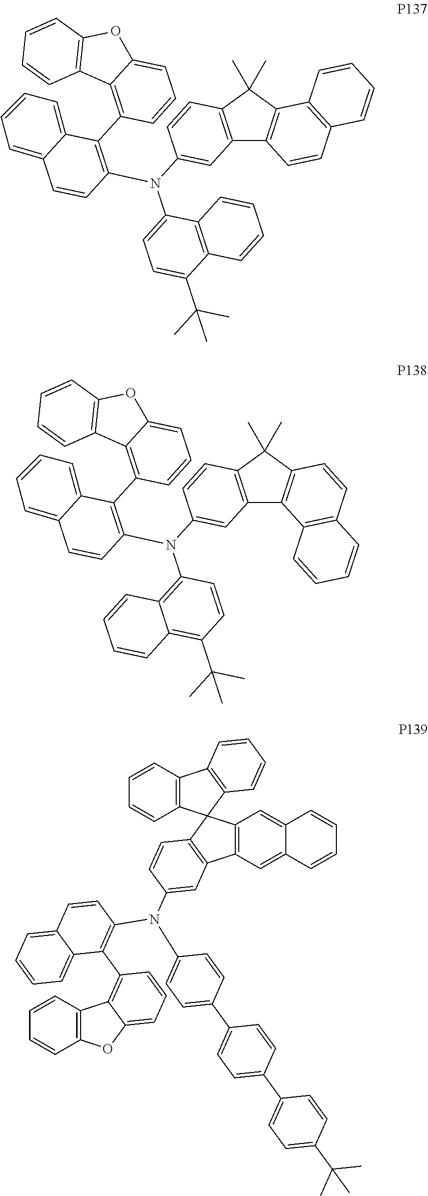

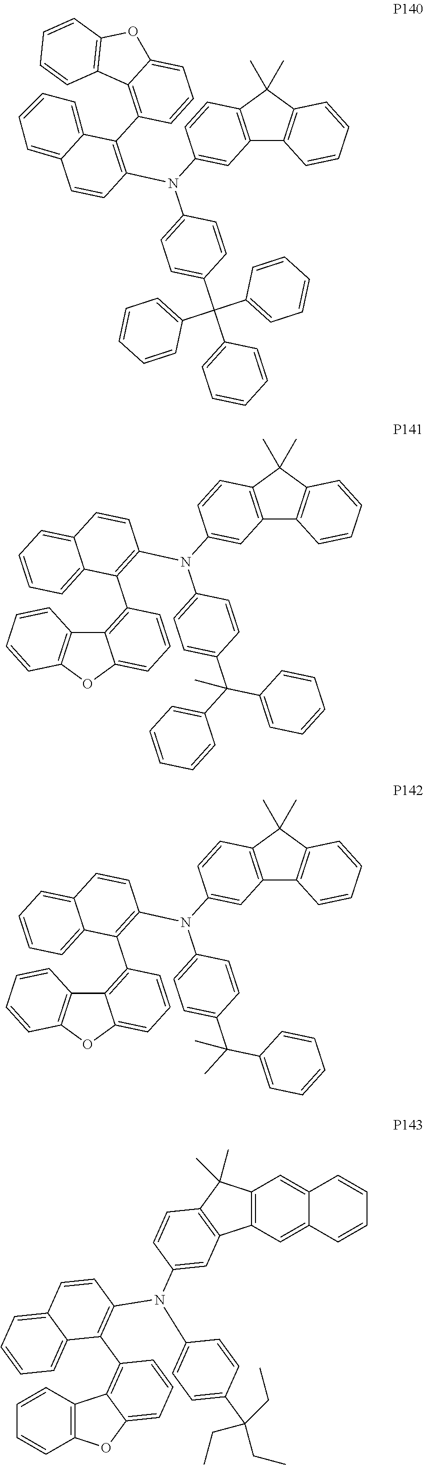

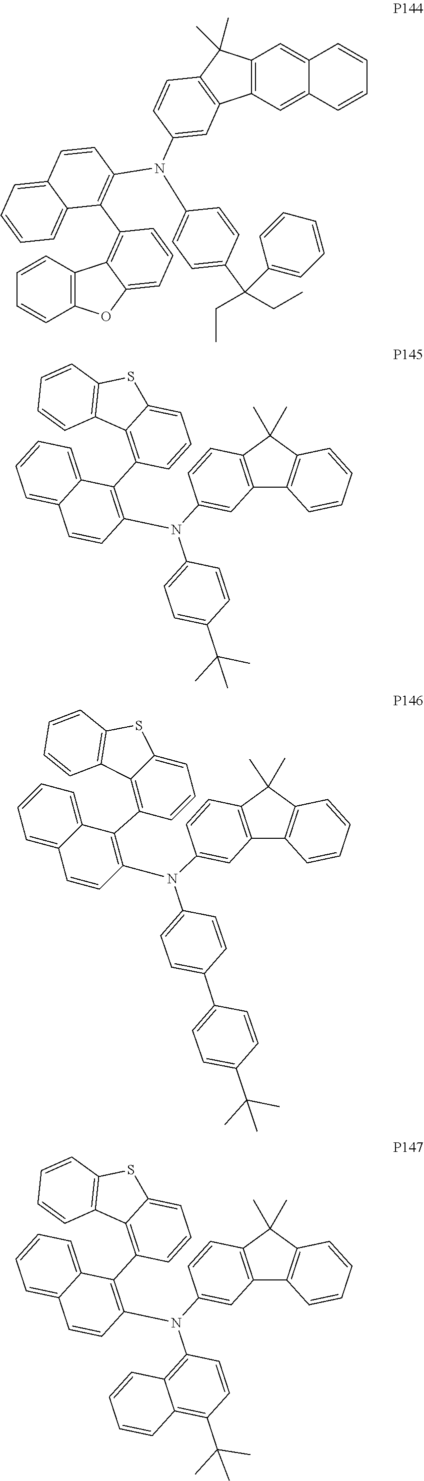 [SMILES: CCC(CC)(c1ccccc1)c1ccc(N(c2ccc3c(c2)-c2cc4ccccc4cc2C3(C)C)c2ccc3ccccc3c2-c2cccc3oc4ccccc4c23)cc1 ; CC(C)(C)c1ccc(N(c2ccc3c(c2)-c2ccccc2C3(C)C)c2ccc3ccccc3c2-c2cccc3sc4ccccc4c23)cc1 ; CC(C)(C)c1ccc(-c2ccc(N(c3ccc4c(c3)-c3ccccc3C4(C)C)c3ccc4ccccc4c3-c3cccc4sc5ccccc5c34)cc2)cc1 ; CC(C)(C)c1ccc(N(c2ccc3c(c2)-c2ccccc2C3(C)C)c2ccc3ccccc3c2-c2cccc3sc4ccccc4c23)c2ccccc12]

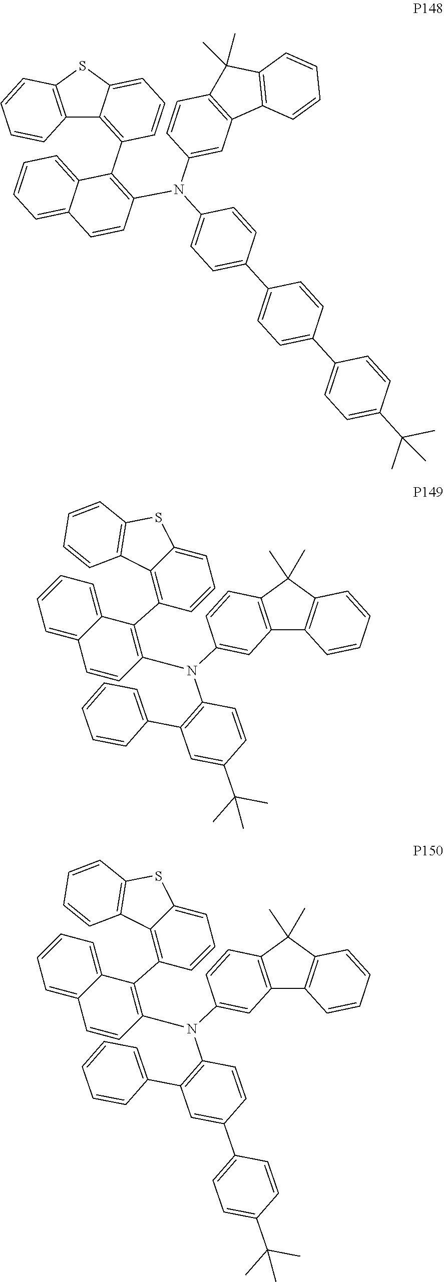 [SMILES: CC(C)(C)c1ccc(-c2ccc(-c3ccc(N(c4ccc5c(c4)-c4ccccc4C5(C)C)c4ccc5ccccc5c4-c4cccc5sc6ccccc6c45)cc3)cc2)cc1 ; CC(C)(C)c1ccc(N(c2ccc3c(c2)-c2ccccc2C3(C)C)c2ccc3ccccc3c2-c2cccc3sc4ccccc4c23)c(-c2ccccc2)c1 ; CC(C)(C)c1ccc(-c2ccc(N(c3ccc4c(c3)-c3ccccc3C4(C)C)c3ccc4ccccc4c3-c3cccc4sc5ccccc5c34)c(-c3ccccc3)c2)cc1]

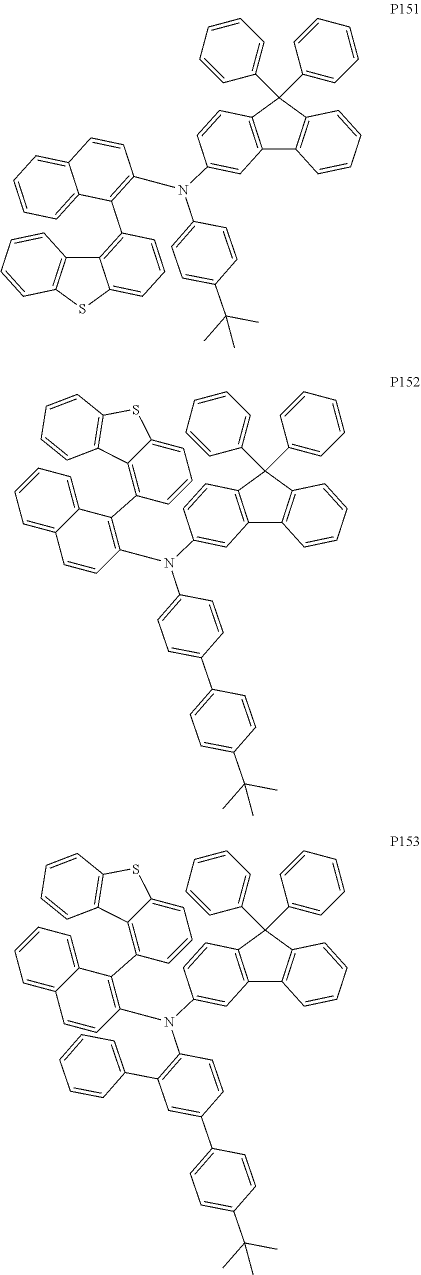

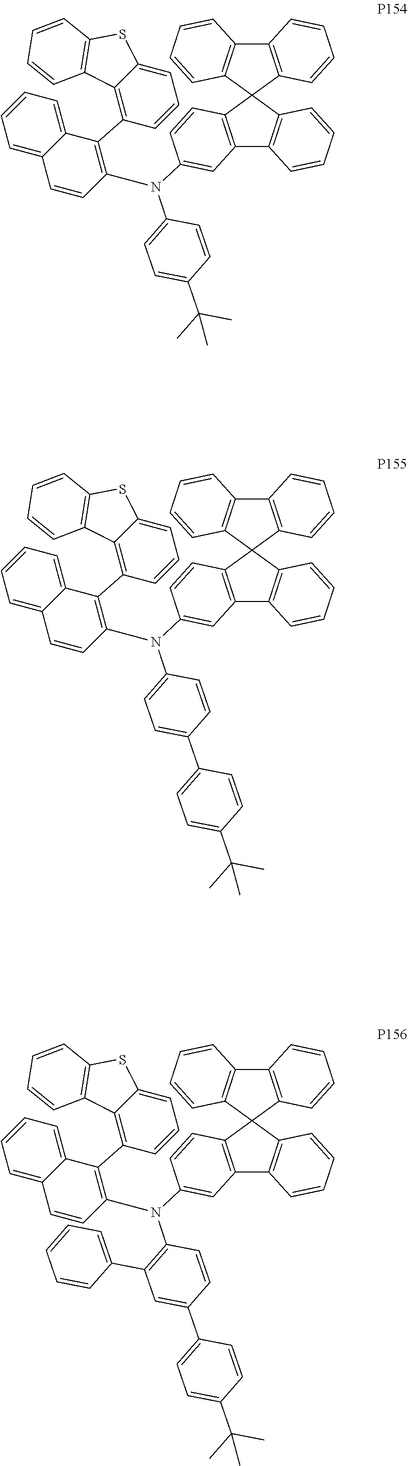 [SMILES: CC(C)(C)c1ccc(N(c2ccc3c(c2)-c2ccccc2C32c3ccccc3-c3ccccc32)c2ccc3ccccc3c2-c2cccc3sc4ccccc4c23)cc1 ; CC(C)(C)c1ccc(-c2ccc(N(c3ccc4c(c3)-c3ccccc3C43c4ccccc4-c4ccccc43)c3ccc4ccccc4c3-c3cccc4sc5ccccc5c34)cc2)cc1 ; CC(C)(C)c1ccc(-c2ccc(N(c3ccc4c(c3)-c3ccccc3C43c4ccccc4-c4ccccc43)c3ccc4ccccc4c3-c3cccc4sc5ccccc5c34)c(-c3ccccc3)c2)cc1]

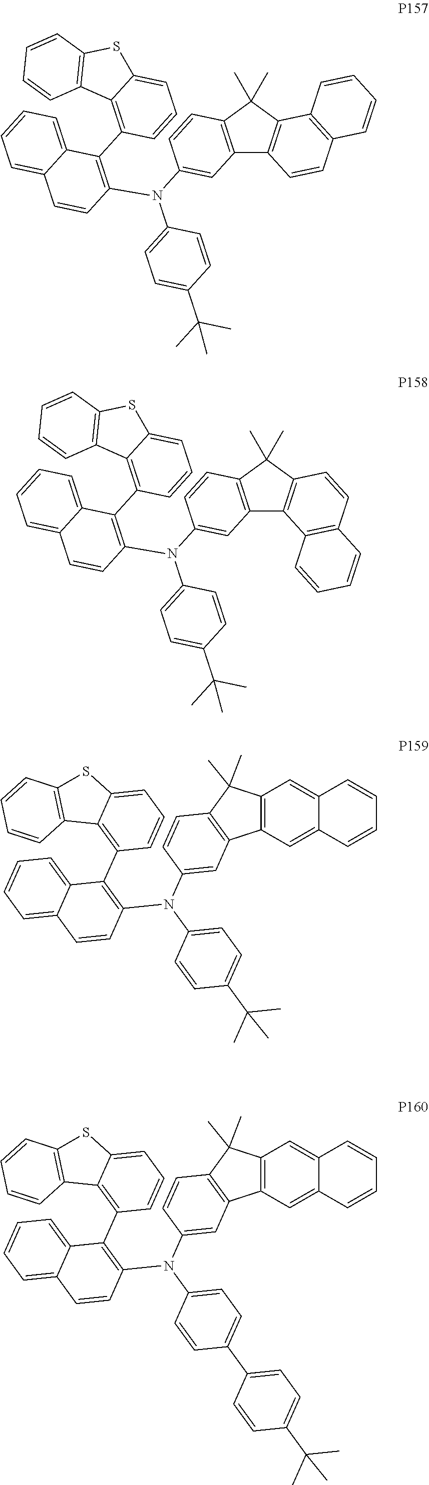

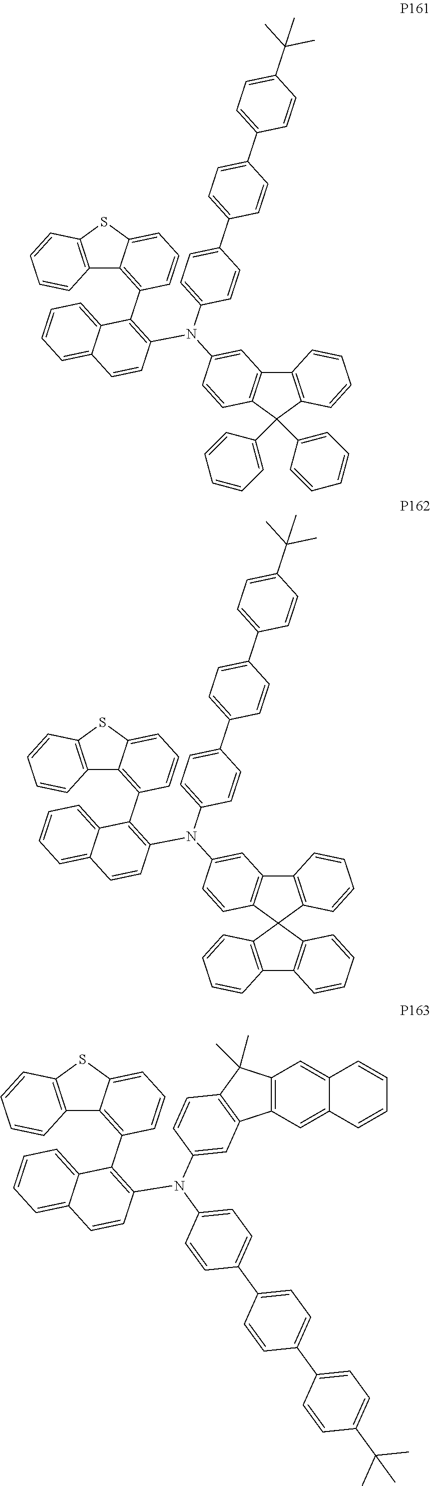

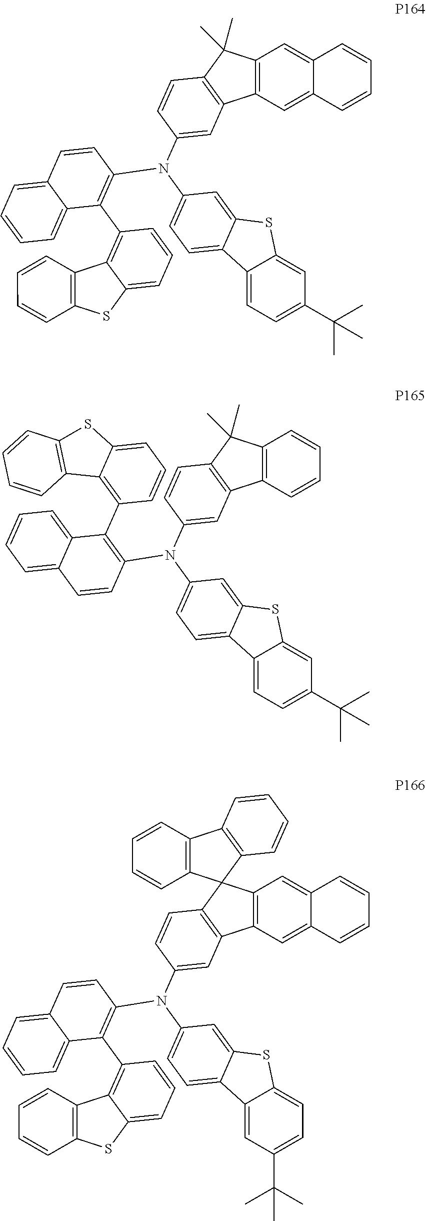 [SMILES: CC(C)(C)c1ccc2c(c1)sc1cc(N(c3ccc4c(c3)-c3cc5ccccc5cc3C4(C)C)c3ccc4ccccc4c3-c3cccc4sc5ccccc5c34)ccc12 ; CC(C)(C)c1ccc2c(c1)sc1cc(N(c3ccc4c(c3)-c3ccccc3C4(C)C)c3ccc4ccccc4c3-c3cccc4sc5ccccc5c34)ccc12 ; CC(C)(C)c1ccc2sc3cc(N(c4ccc5c(c4)-c4cc6ccccc6cc4C54c5ccccc5-c5ccccc54)c4ccc5ccccc5c4-c4cccc5sc6ccccc6c45)ccc3c2c1]

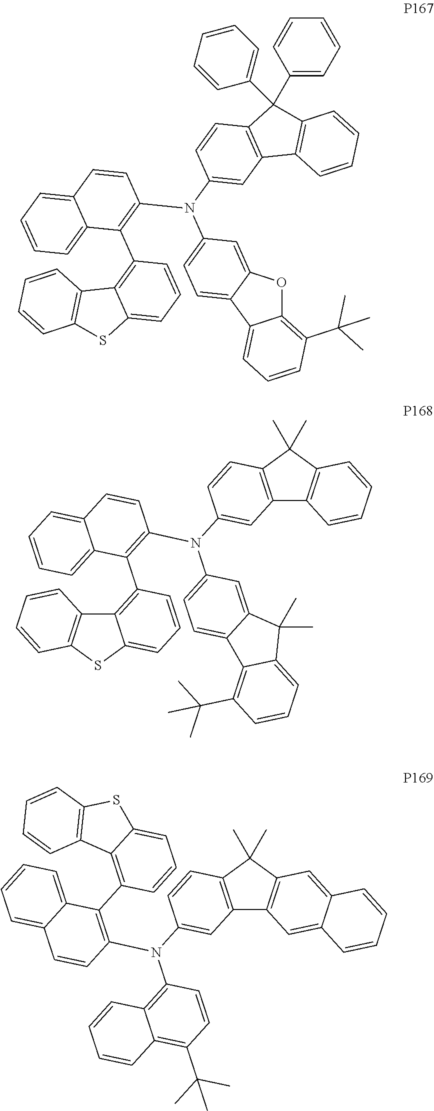

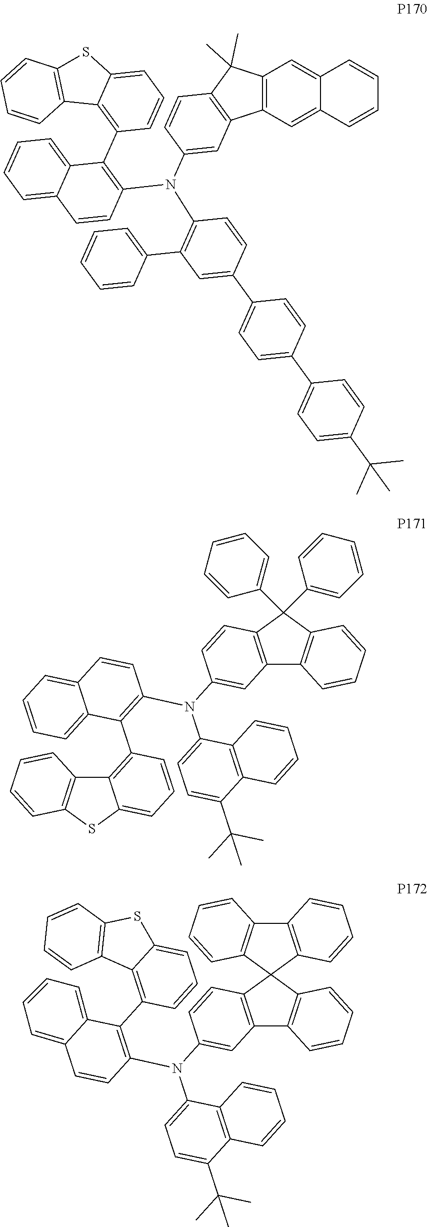

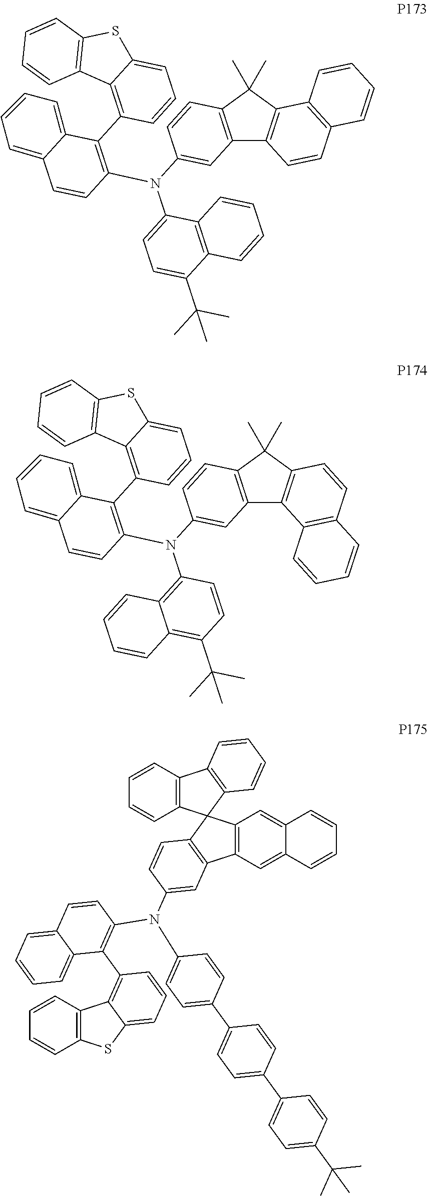

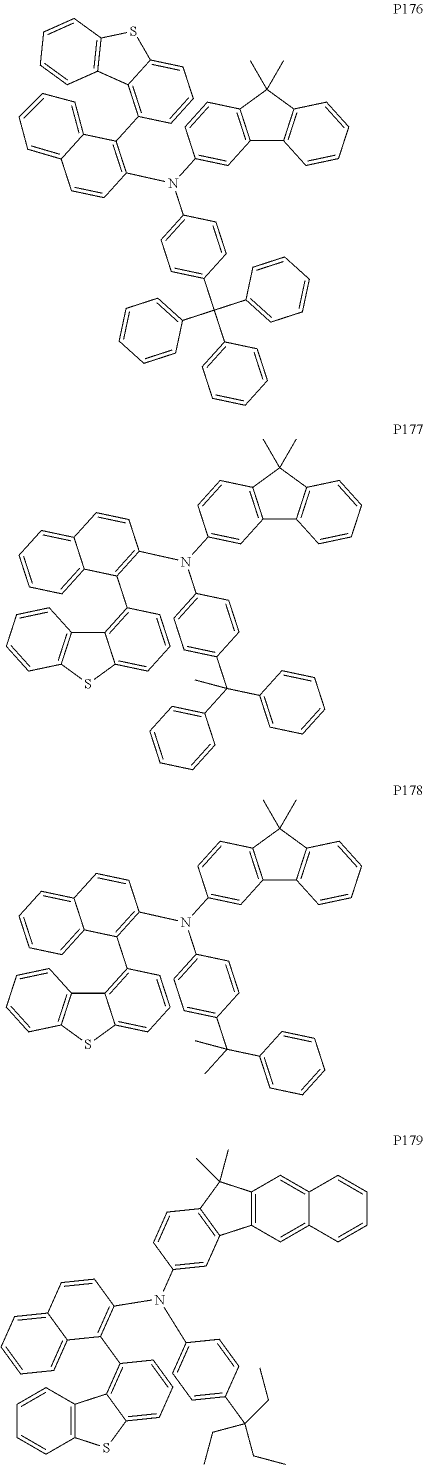

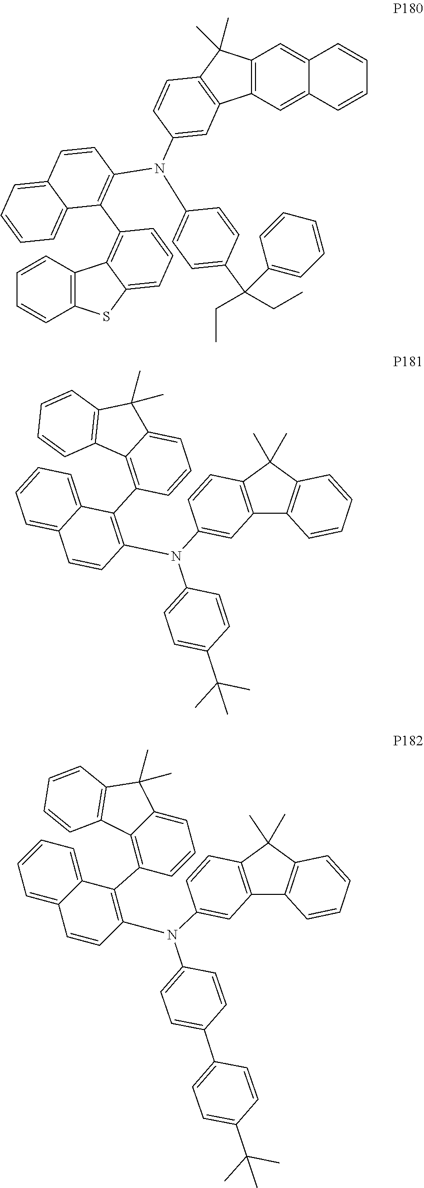 [SMILES: CCC(CC)(c1ccccc1)c1ccc(N(c2ccc3c(c2)-c2cc4ccccc4cc2C3(C)C)c2ccc3ccccc3c2-c2cccc3sc4ccccc4c23)cc1 ; CC(C)(C)c1ccc(N(c2ccc3c(c2)-c2ccccc2C3(C)C)c2ccc3ccccc3c2-c2cccc3c2-c2ccccc2C3(C)C)cc1 ; CC(C)(C)c1ccc(-c2ccc(N(c3ccc4c(c3)-c3ccccc3C4(C)C)c3ccc4ccccc4c3-c3cccc4c3-c3ccccc3C4(C)C)cc2)cc1]

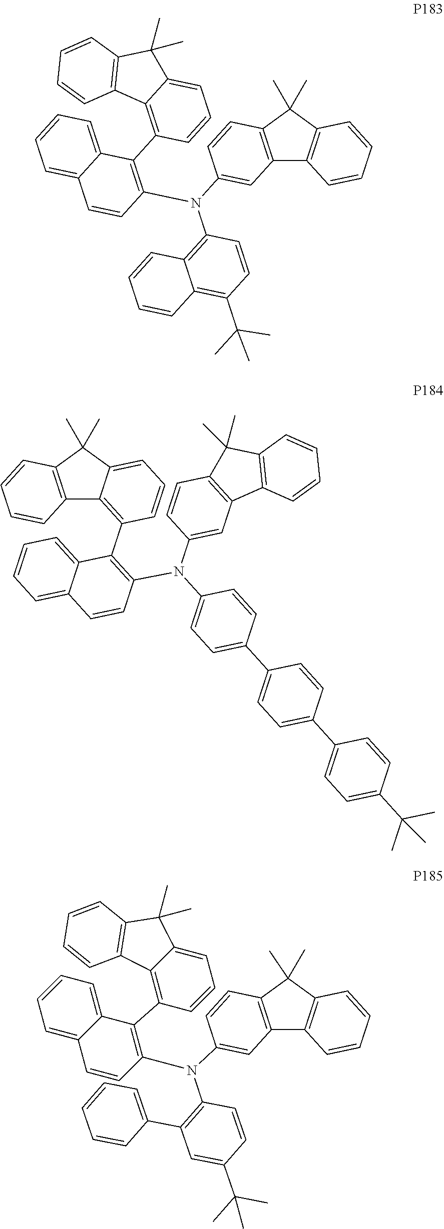 [SMILES: CC(C)(C)c1ccc(N(c2ccc3c(c2)-c2ccccc2C3(C)C)c2ccc3ccccc3c2-c2cccc3c2-c2ccccc2C3(C)C)c2ccccc12 ; CC(C)(C)c1ccc(-c2ccc(-c3ccc(N(c4ccc5c(c4)-c4ccccc4C5(C)C)c4ccc5ccccc5c4-c4cccc5c4-c4ccccc4C5(C)C)cc3)cc2)cc1 ; CC(C)(C)c1ccc(N(c2ccc3c(c2)-c2ccccc2C3(C)C)c2ccc3ccccc3c2-c2cccc3c2-c2ccccc2C3(C)C)c(-c2ccccc2)c1]

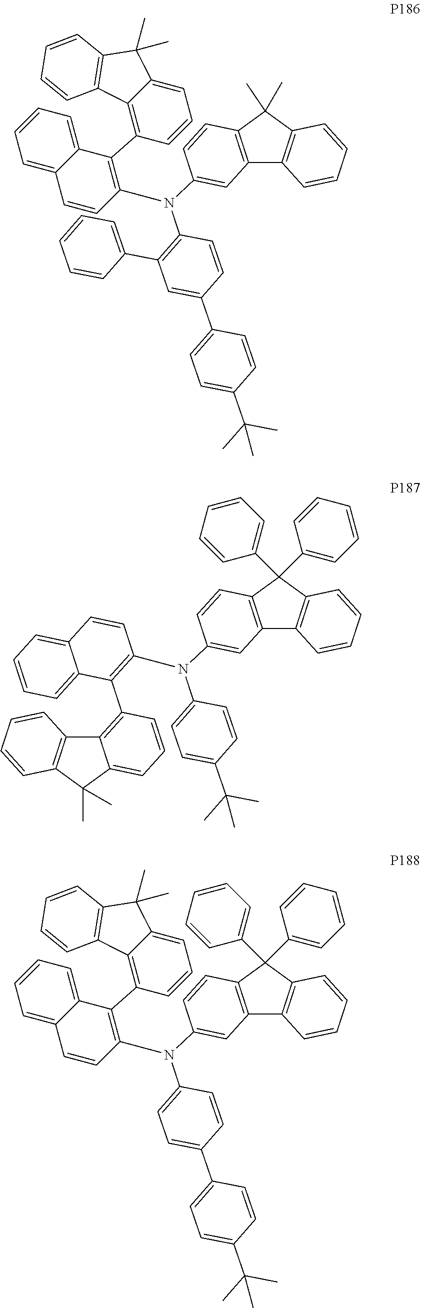

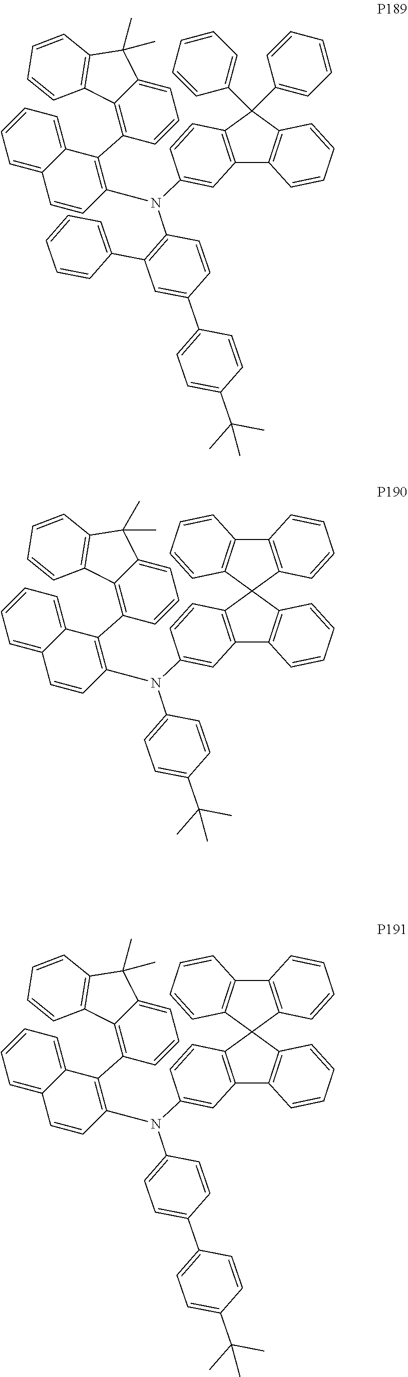 [SMILES: CC(C)(C)c1ccc(-c2ccc(N(c3ccc4c(c3)-c3ccccc3C4(c3ccccc3)c3ccccc3)c3ccc4ccccc4c3-c3cccc4c3-c3ccccc3C4(C)C)c(-c3ccccc3)c2)cc1 ; CC(C)(C)c1ccc(N(c2ccc3c(c2)-c2ccccc2C32c3ccccc3-c3ccccc32)c2ccc3ccccc3c2-c2cccc3c2-c2ccccc2C3(C)C)cc1 ; CC(C)(C)c1ccc(-c2ccc(N(c3ccc4c(c3)-c3ccccc3C43c4ccccc4-c4ccccc43)c3ccc4ccccc4c3-c3cccc4c3-c3ccccc3C4(C)C)cc2)cc1]

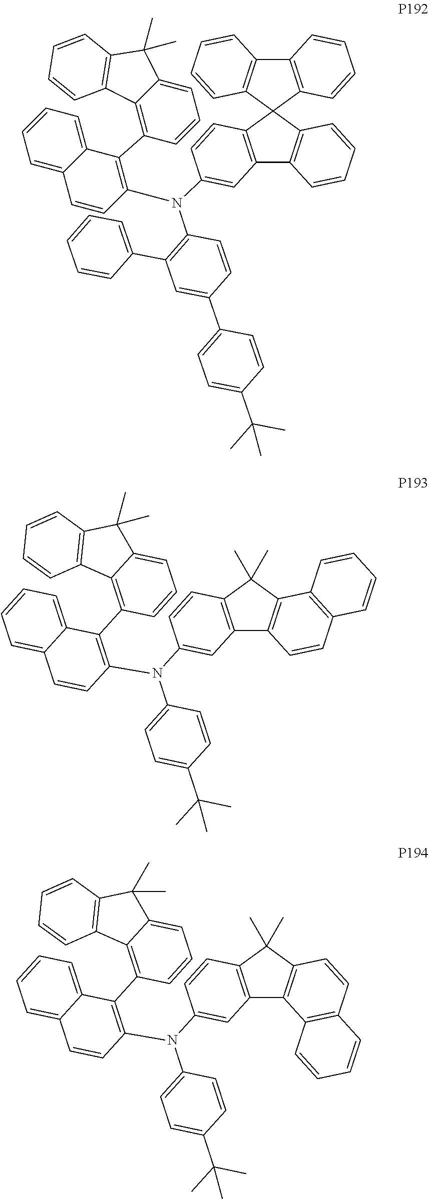

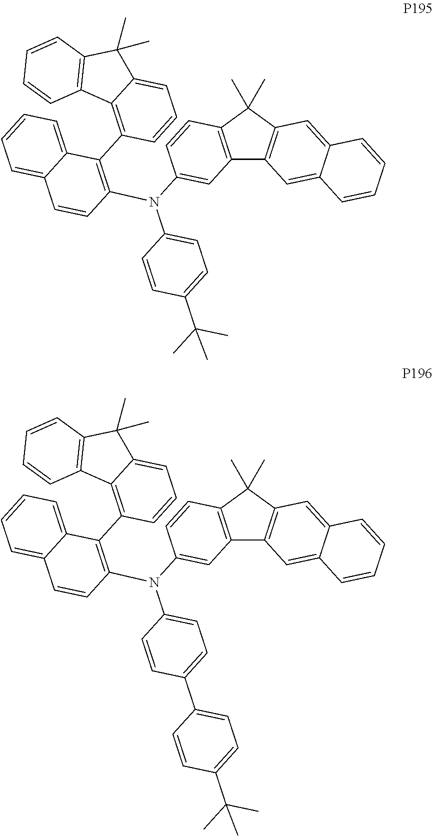 [SMILES: CC(C)(C)c1ccc(N(c2ccc3c(c2)-c2cc4ccccc4cc2C3(C)C)c2ccc3ccccc3c2-c2cccc3c2-c2ccccc2C3(C)C)cc1 ; CC(C)(C)c1ccc(-c2ccc(N(c3ccc4c(c3)-c3cc5ccccc5cc3C4(C)C)c3ccc4ccccc4c3-c3cccc4c3-c3ccccc3C4(C)C)cc2)cc1]

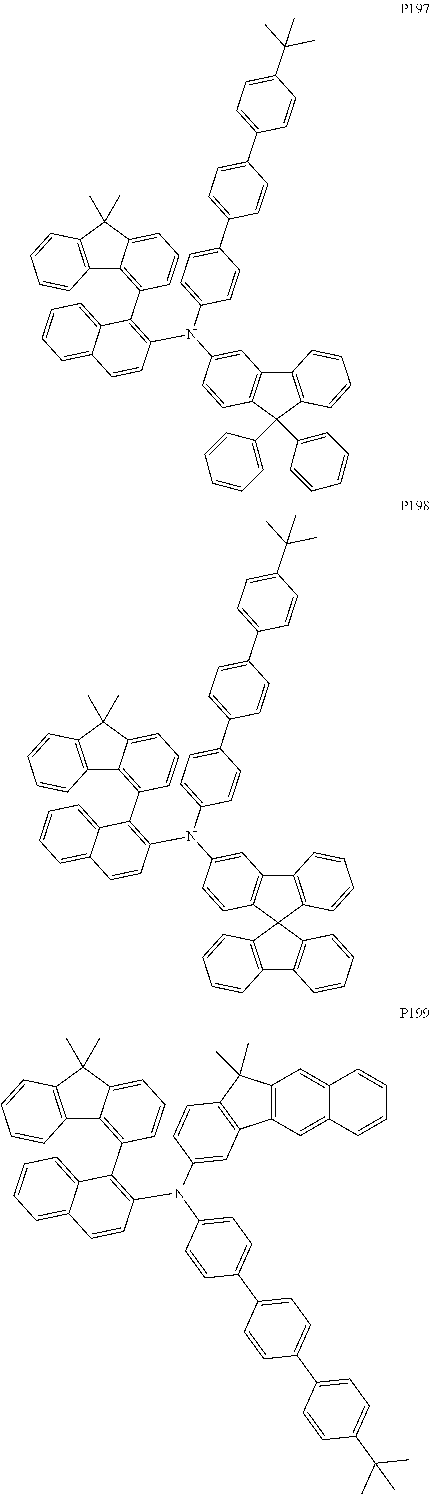

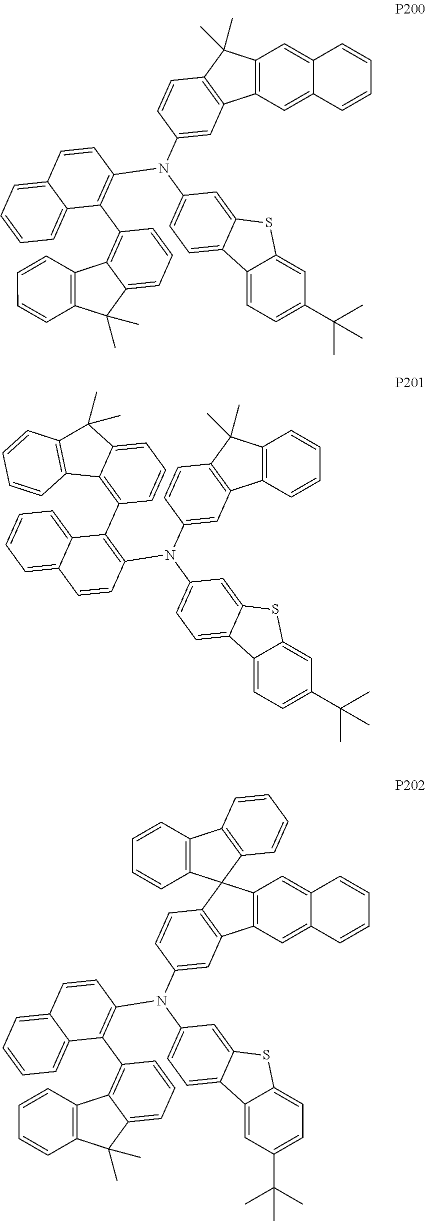 [SMILES: CC(C)(C)c1ccc2c(c1)sc1cc(N(c3ccc4c(c3)-c3cc5ccccc5cc3C4(C)C)c3ccc4ccccc4c3-c3cccc4c3-c3ccccc3C4(C)C)ccc12 ; CC(C)(C)c1ccc2c(c1)sc1cc(N(c3ccc4c(c3)-c3ccccc3C4(C)C)c3ccc4ccccc4c3-c3cccc4c3-c3ccccc3C4(C)C)ccc12 ; CC(C)(C)c1ccc2sc3cc(N(c4ccc5c(c4)-c4cc6ccccc6cc4C54c5ccccc5-c5ccccc54)c4ccc5ccccc5c4-c4cccc5c4-c4ccccc4C5(C)C)ccc3c2c1]

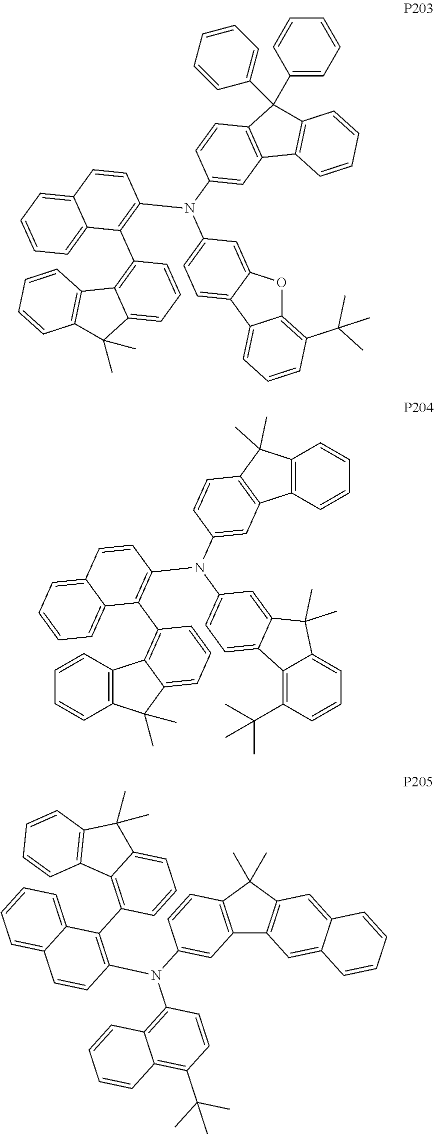

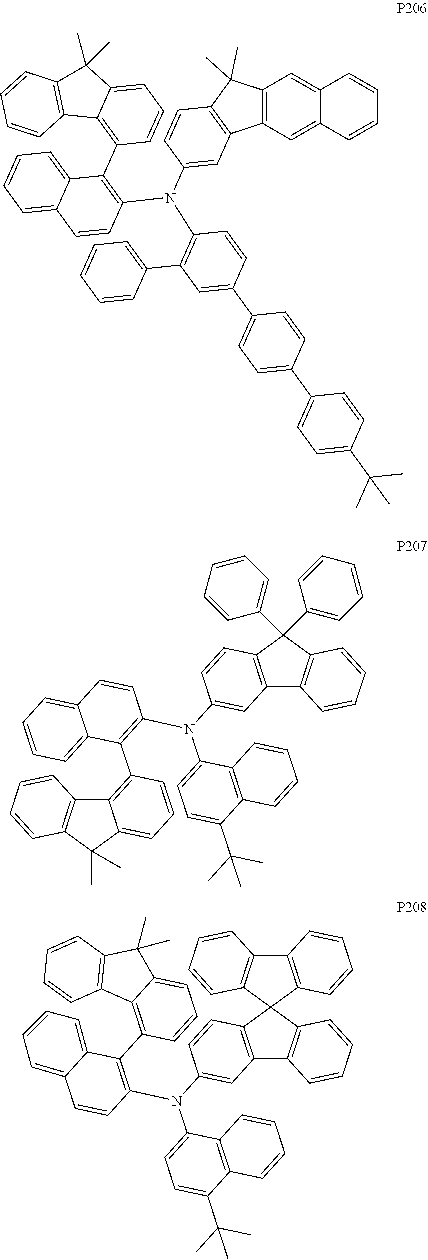

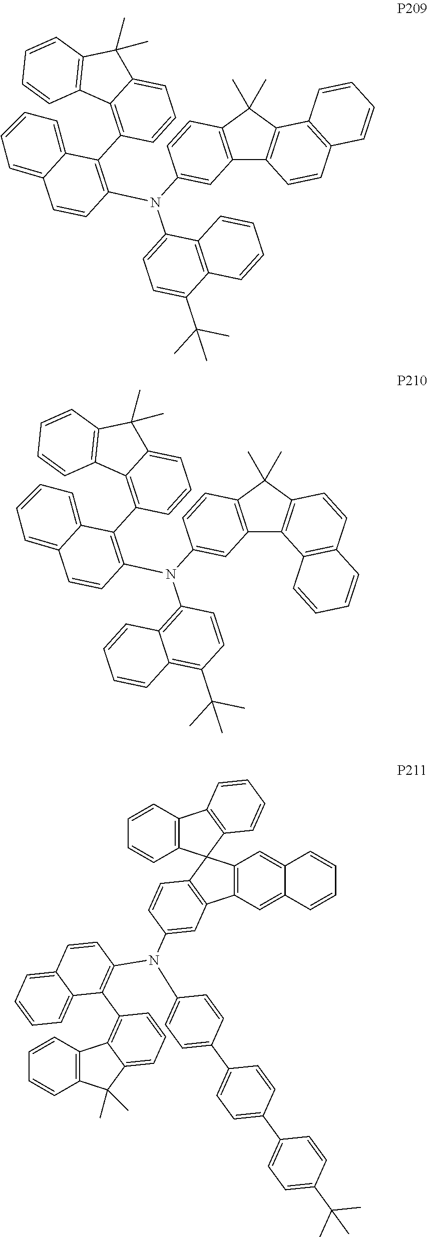 [SMILES: CC(C)(C)c1ccc(N(c2ccc3c(c2)-c2ccc4ccccc4c2C3(C)C)c2ccc3ccccc3c2-c2cccc3c2-c2ccccc2C3(C)C)c2ccccc12 ; CC(C)(C)c1ccc(N(c2ccc3c(c2)-c2c(ccc4ccccc24)C3(C)C)c2ccc3ccccc3c2-c2cccc3c2-c2ccccc2C3(C)C)c2ccccc12 ; CC(C)(C)c1ccc(-c2ccc(-c3ccc(N(c4ccc5c(c4)-c4cc6ccccc6cc4C54c5ccccc5-c5ccccc54)c4ccc5ccccc5c4-c4cccc5c4-c4ccccc4C5(C)C)cc3)cc2)cc1]

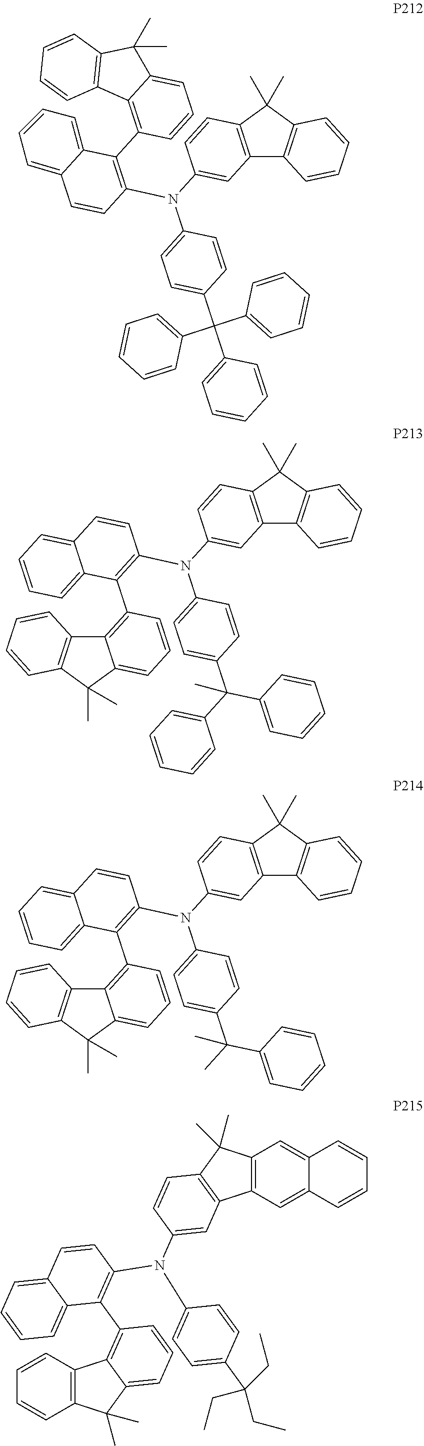

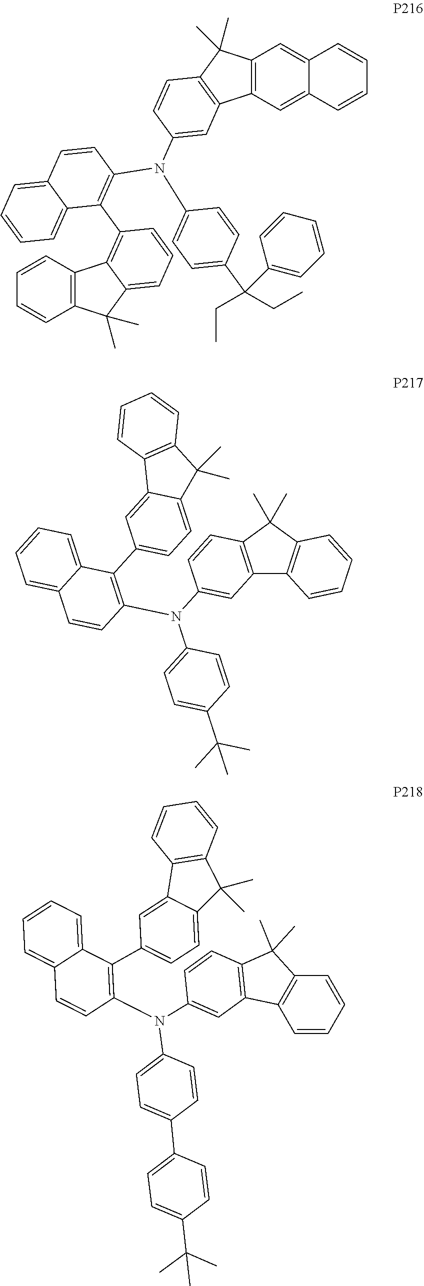

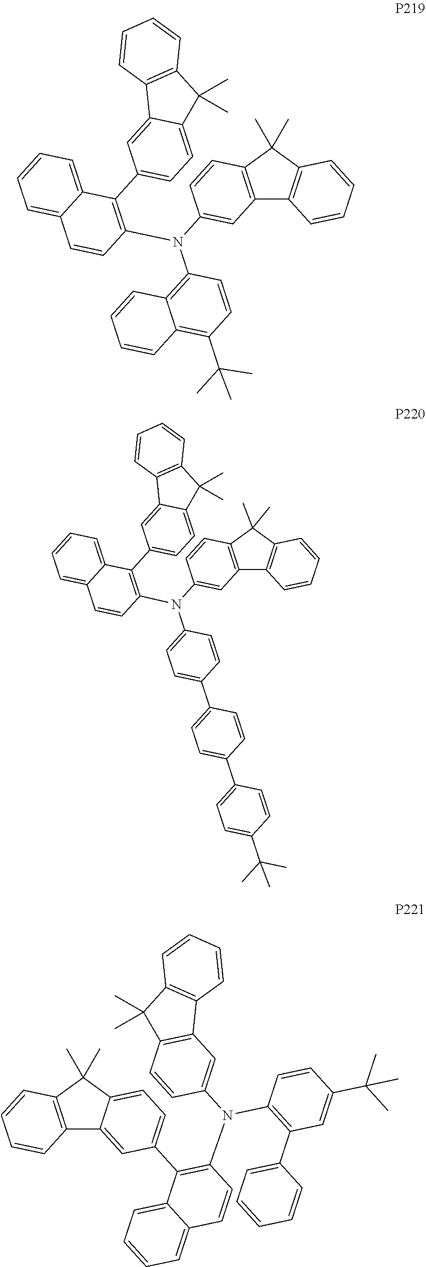

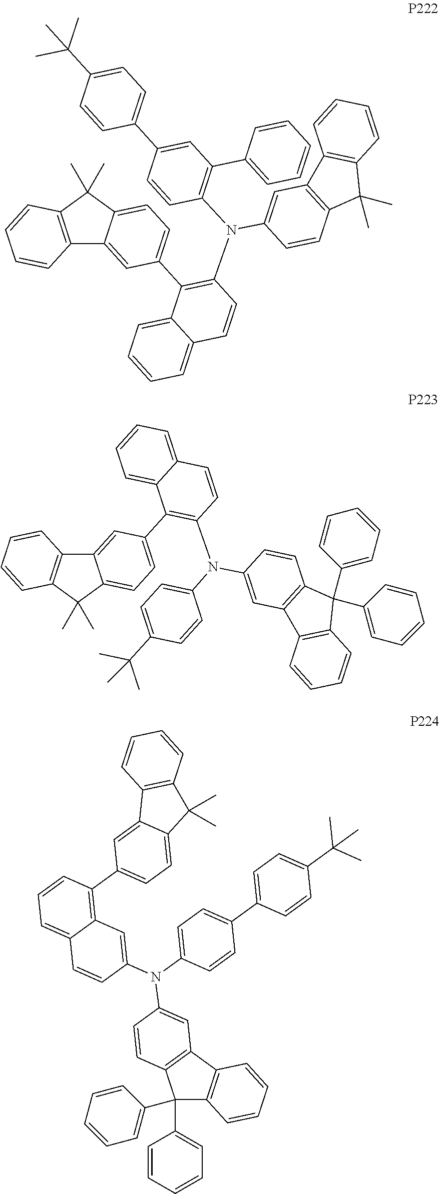 [SMILES: CC(C)(C)c1ccc(-c2ccc(N(c3ccc4c(c3)-c3ccccc3C4(C)C)c3ccc4ccccc4c3-c3ccc4c(c3)-c3ccccc3C4(C)C)c(-c3ccccc3)c2)cc1 ; CC(C)(C)c1ccc(N(c2ccc3c(c2)-c2ccccc2C3(c2ccccc2)c2ccccc2)c2ccc3ccccc3c2-c2ccc3c(c2)-c2ccccc2C3(C)C)cc1 ; CC(C)(C)c1ccc(-c2ccc(N(c3ccc4c(c3)-c3ccccc3C4(c3ccccc3)c3ccccc3)c3ccc4cccc(-c5ccc6c(c5)-c5ccccc5C6(C)C)c4c3)cc2)cc1]

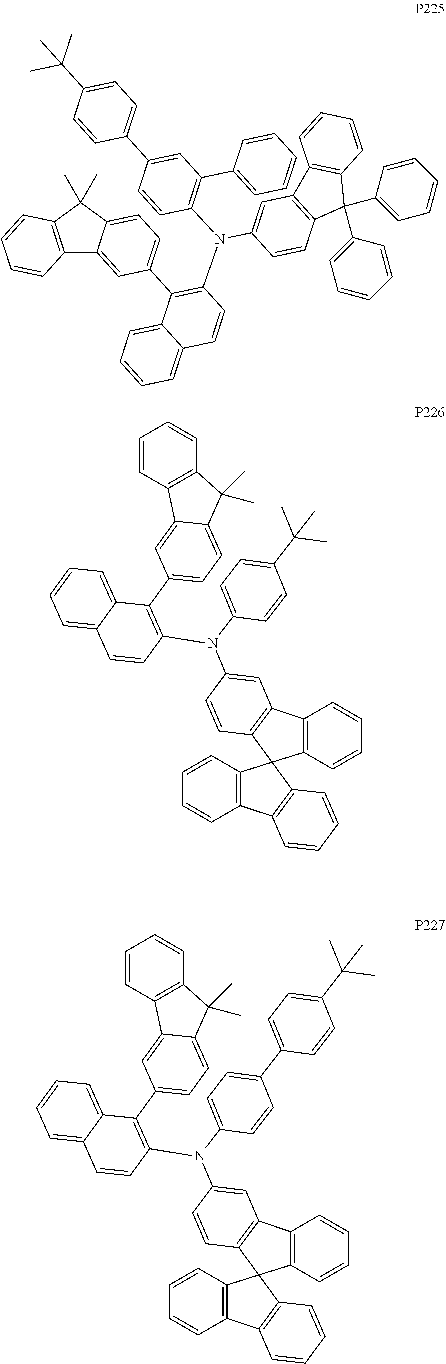

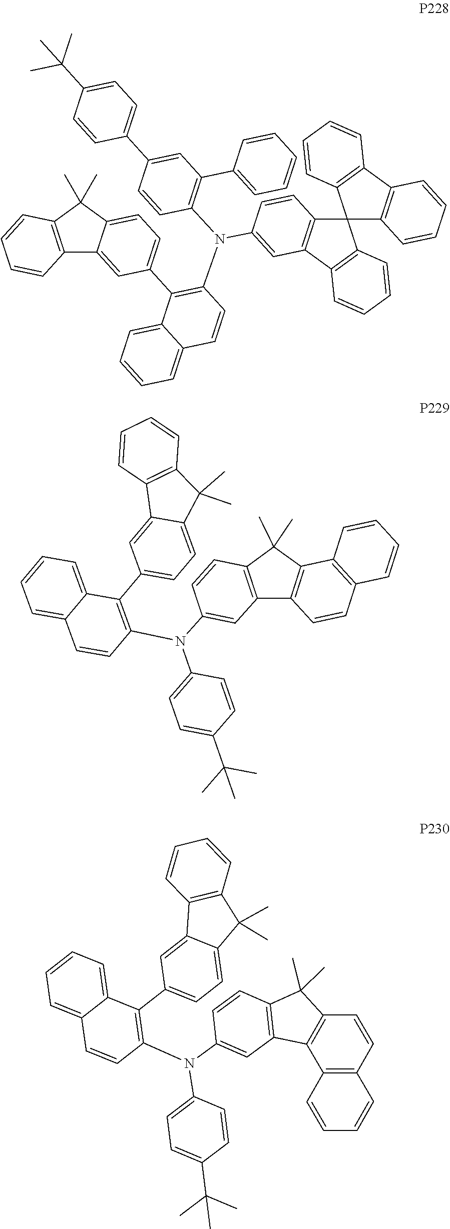

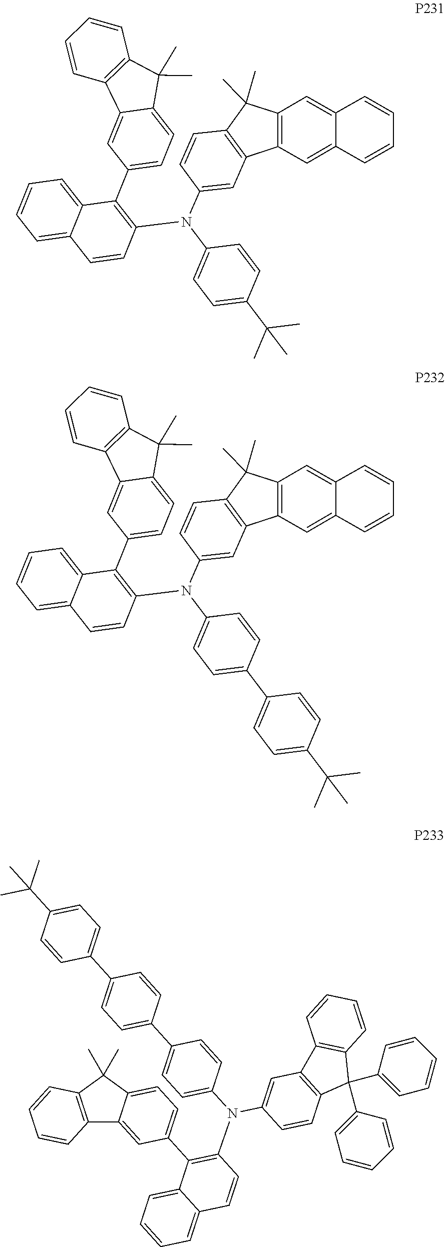 [SMILES: CC(C)(C)c1ccc(N(c2ccc3c(c2)-c2cc4ccccc4cc2C3(C)C)c2ccc3ccccc3c2-c2ccc3c(c2)-c2ccccc2C3(C)C)cc1 ; CC(C)(C)c1ccc(-c2ccc(N(c3ccc4c(c3)-c3cc5ccccc5cc3C4(C)C)c3ccc4ccccc4c3-c3ccc4c(c3)-c3ccccc3C4(C)C)cc2)cc1 ; CC(C)(C)c1ccc(-c2ccc(-c3ccc(N(c4ccc5c(c4)-c4ccccc4C5(c4ccccc4)c4ccccc4)c4ccc5ccccc5c4-c4ccc5c(c4)-c4ccccc4C5(C)C)cc3)cc2)cc1]

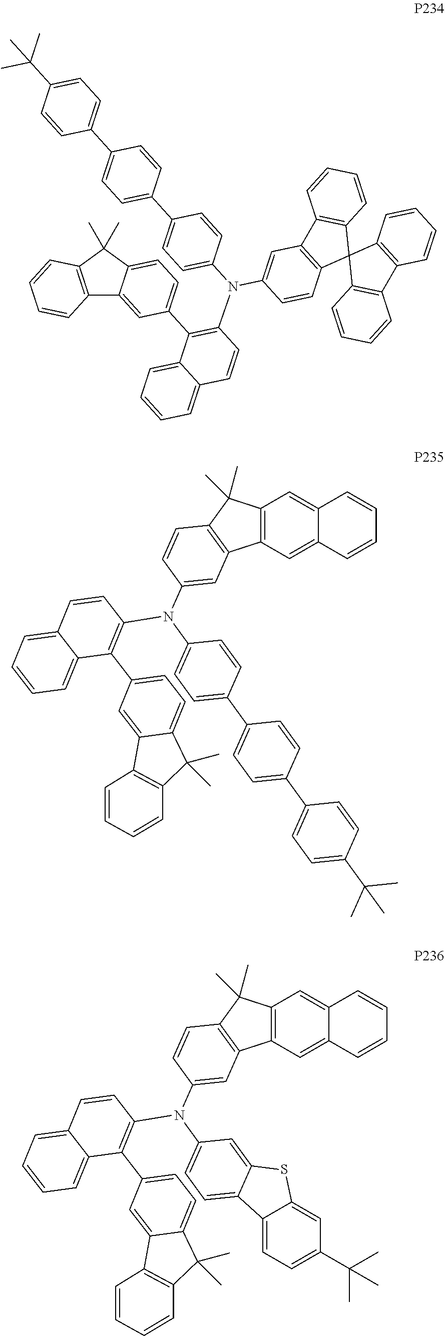 [SMILES: CC(C)(C)c1ccc(-c2ccc(-c3ccc(N(c4ccc5c(c4)-c4ccccc4C54c5ccccc5-c5ccccc54)c4ccc5ccccc5c4-c4ccc5c(c4)-c4ccccc4C5(C)C)cc3)cc2)cc1 ; CC(C)(C)c1ccc(-c2ccc(-c3ccc(N(c4ccc5c(c4)-c4cc6ccccc6cc4C5(C)C)c4ccc5ccccc5c4-c4ccc5c(c4)-c4ccccc4C5(C)C)cc3)cc2)cc1 ; CC(C)(C)c1ccc2c(c1)sc1cc(N(c3ccc4c(c3)-c3cc5ccccc5cc3C4(C)C)c3ccc4ccccc4c3-c3ccc4c(c3)-c3ccccc3C4(C)C)ccc12]

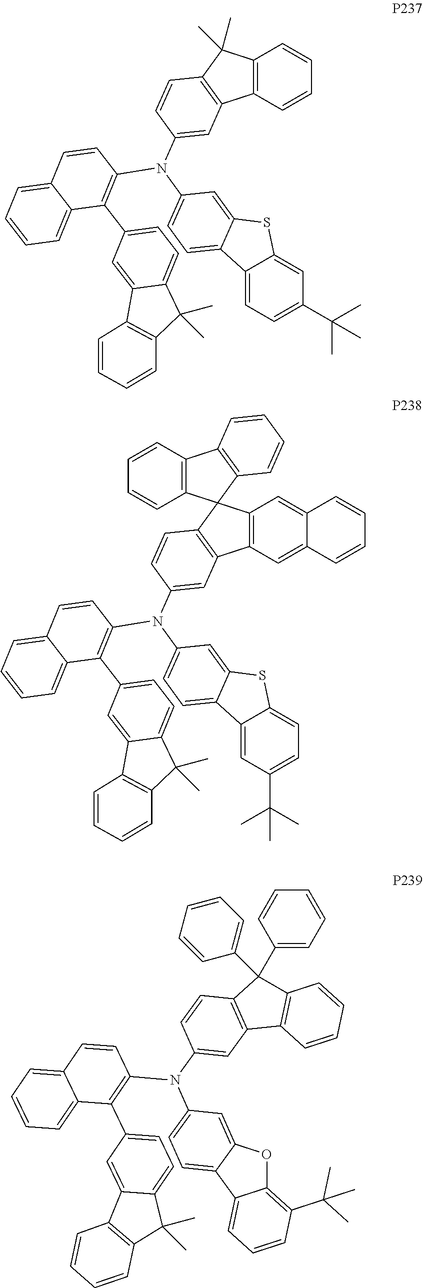 [SMILES: CC(C)(C)c1ccc2c(c1)sc1cc(N(c3ccc4c(c3)-c3ccccc3C4(C)C)c3ccc4ccccc4c3-c3ccc4c(c3)-c3ccccc3C4(C)C)ccc12 ; CC(C)(C)c1ccc2sc3cc(N(c4ccc5c(c4)-c4cc6ccccc6cc4C54c5ccccc5-c5ccccc54)c4ccc5ccccc5c4-c4ccc5c(c4)-c4ccccc4C5(C)C)ccc3c2c1 ; CC(C)(C)c1cccc2c1oc1cc(N(c3ccc4c(c3)-c3ccccc3C4(c3ccccc3)c3ccccc3)c3ccc4ccccc4c3-c3ccc4c(c3)-c3ccccc3C4(C)C)ccc12]

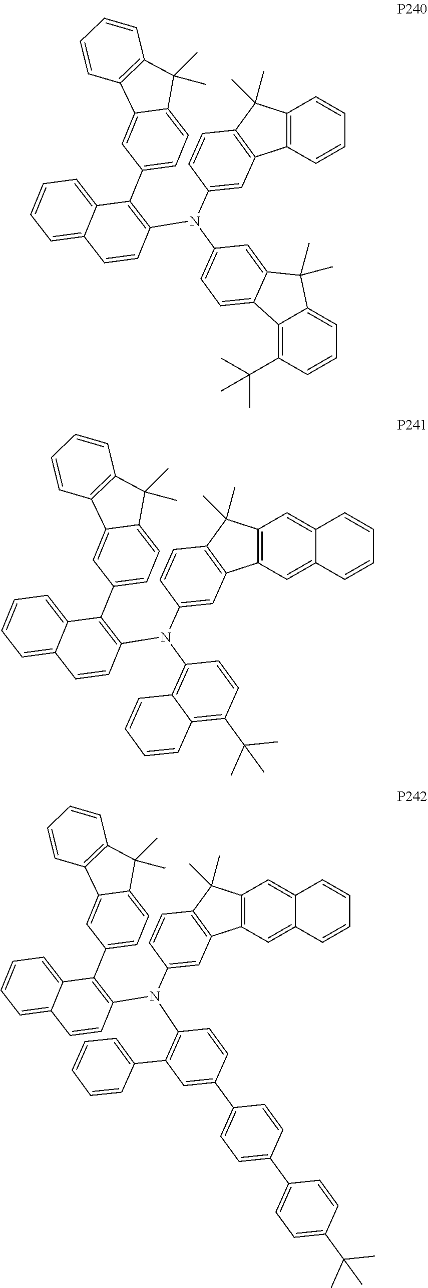 [SMILES: CC(C)(C)c1cccc2c1-c1ccc(N(c3ccc4c(c3)-c3ccccc3C4(C)C)c3ccc4ccccc4c3-c3ccc4c(c3)-c3ccccc3C4(C)C)cc1C2(C)C ; CC(C)(C)c1ccc(N(c2ccc3c(c2)-c2cc4ccccc4cc2C3(C)C)c2ccc3ccccc3c2-c2ccc3c(c2)-c2ccccc2C3(C)C)c2ccccc12 ; CC(C)(C)c1ccc(-c2ccc(-c3ccc(N(c4ccc5c(c4)-c4cc6ccccc6cc4C5(C)C)c4ccc5ccccc5c4-c4ccc5c(c4)-c4ccccc4C5(C)C)c(-c4ccccc4)c3)cc2)cc1]

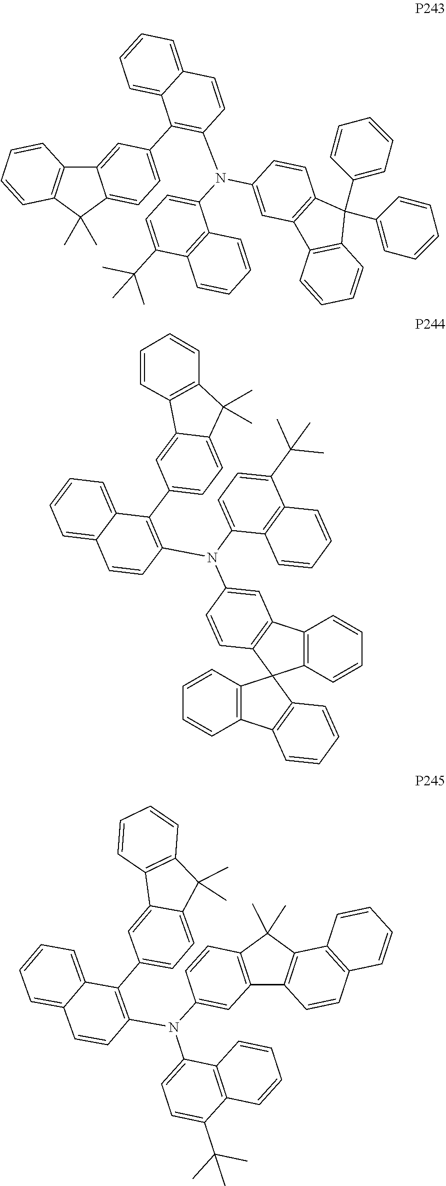

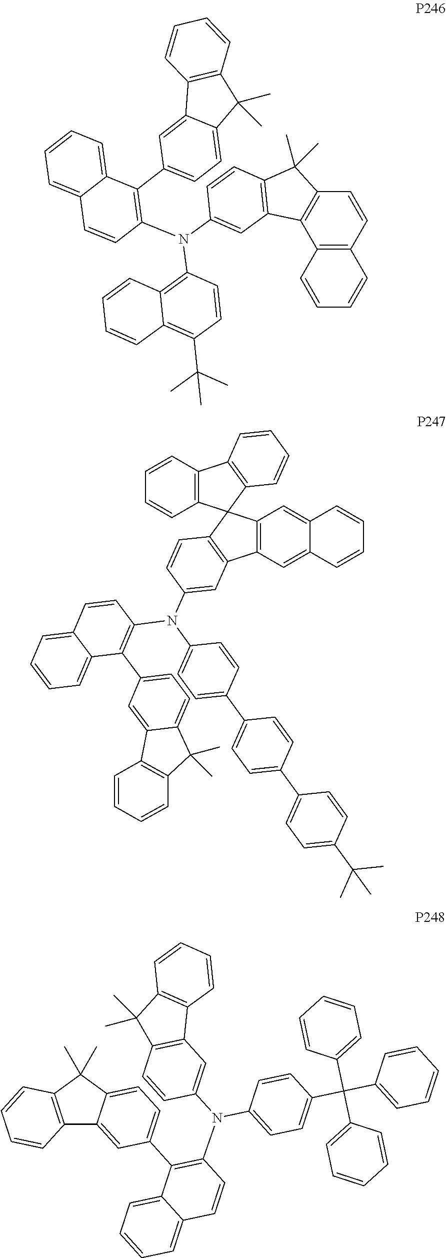 [SMILES: CC(C)(C)c1ccc(N(c2ccc3c(c2)-c2c(ccc4ccccc24)C3(C)C)c2ccc3ccccc3c2-c2ccc3c(c2)-c2ccccc2C3(C)C)c2ccccc12 ; CC(C)(C)c1ccc(-c2ccc(-c3ccc(N(c4ccc5c(c4)-c4cc6ccccc6cc4C54c5ccccc5-c5ccccc54)c4ccc5ccccc5c4-c4ccc5c(c4)-c4ccccc4C5(C)C)cc3)cc2)cc1 ; CC1(C)c2ccccc2-c2cc(-c3c(N(c4ccc(C(c5ccccc5)(c5ccccc5)c5ccccc5)cc4)c4ccc5c(c4)-c4ccccc4C5(C)C)ccc4ccccc34)ccc21]

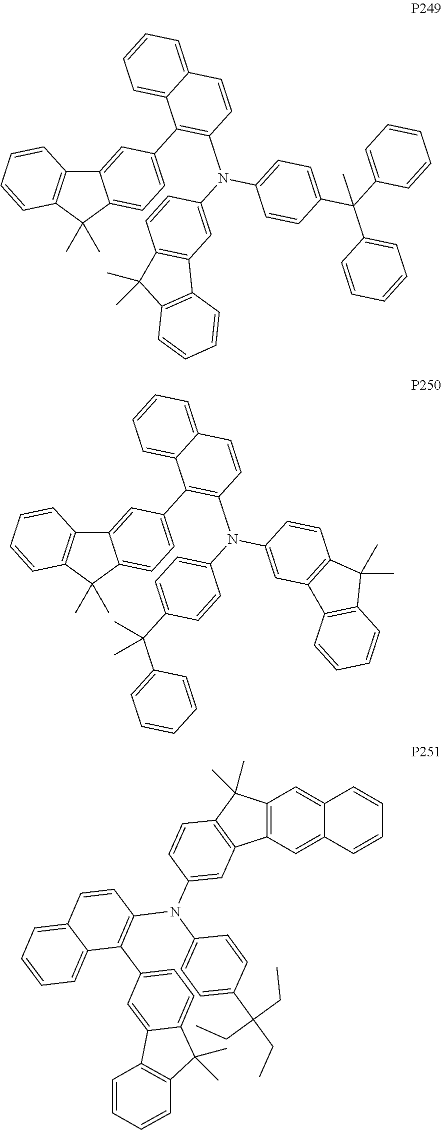

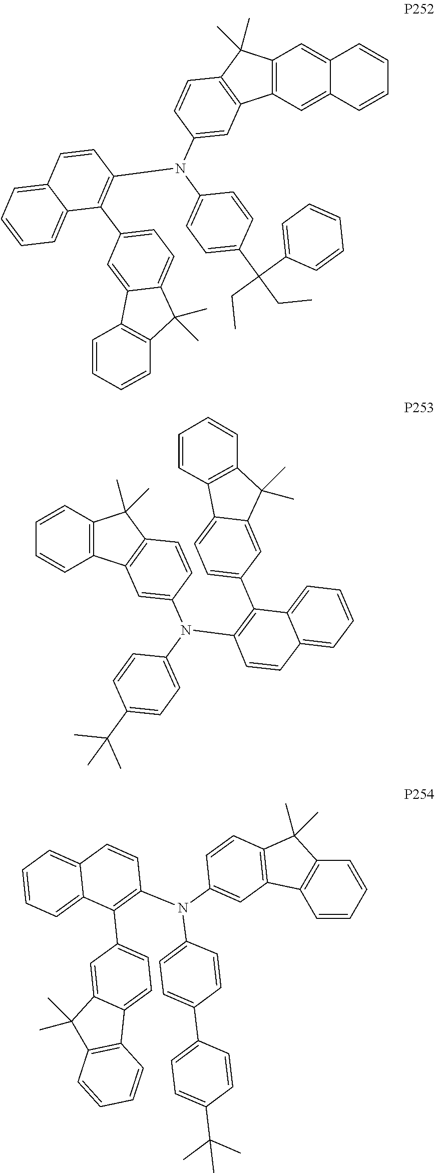 [SMILES: CCC(CC)(c1ccccc1)c1ccc(N(c2ccc3c(c2)-c2cc4ccccc4cc2C3(C)C)c2ccc3ccccc3c2-c2ccc3c(c2)-c2ccccc2C3(C)C)cc1 ; CC(C)(C)c1ccc(N(c2ccc3c(c2)-c2ccccc2C3(C)C)c2ccc3ccccc3c2-c2ccc3c(c2)C(C)(C)c2ccccc2-3)cc1 ; CC(C)(C)c1ccc(-c2ccc(N(c3ccc4c(c3)-c3ccccc3C4(C)C)c3ccc4ccccc4c3-c3ccc4c(c3)C(C)(C)c3ccccc3-4)cc2)cc1]

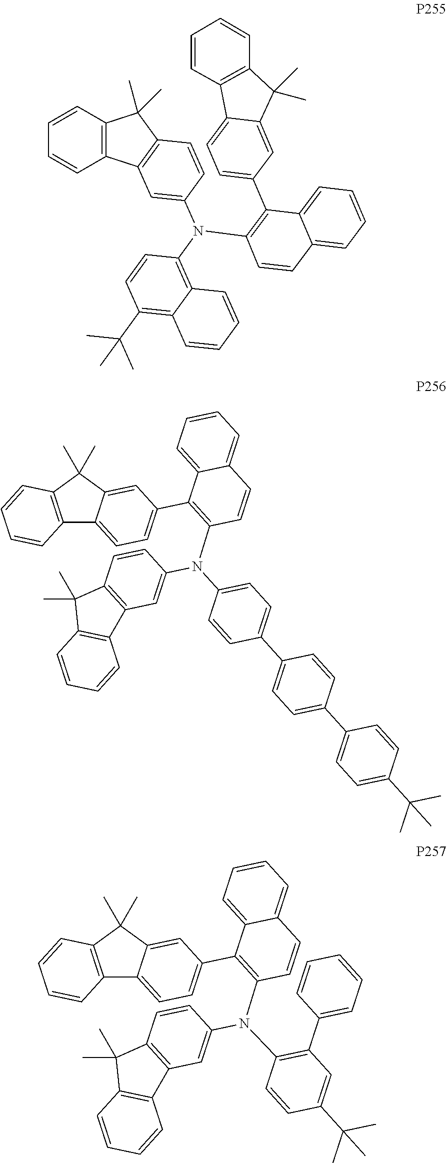 [SMILES: CC(C)(C)c1ccc(N(c2ccc3c(c2)-c2ccccc2C3(C)C)c2ccc3ccccc3c2-c2ccc3c(c2)C(C)(C)c2ccccc2-3)c2ccccc12 ; CC(C)(C)c1ccc(-c2ccc(-c3ccc(N(c4ccc5c(c4)-c4ccccc4C5(C)C)c4ccc5ccccc5c4-c4ccc5c(c4)C(C)(C)c4ccccc4-5)cc3)cc2)cc1 ; CC(C)(C)c1ccc(N(c2ccc3c(c2)-c2ccccc2C3(C)C)c2ccc3ccccc3c2-c2ccc3c(c2)C(C)(C)c2ccccc2-3)c(-c2ccccc2)c1]

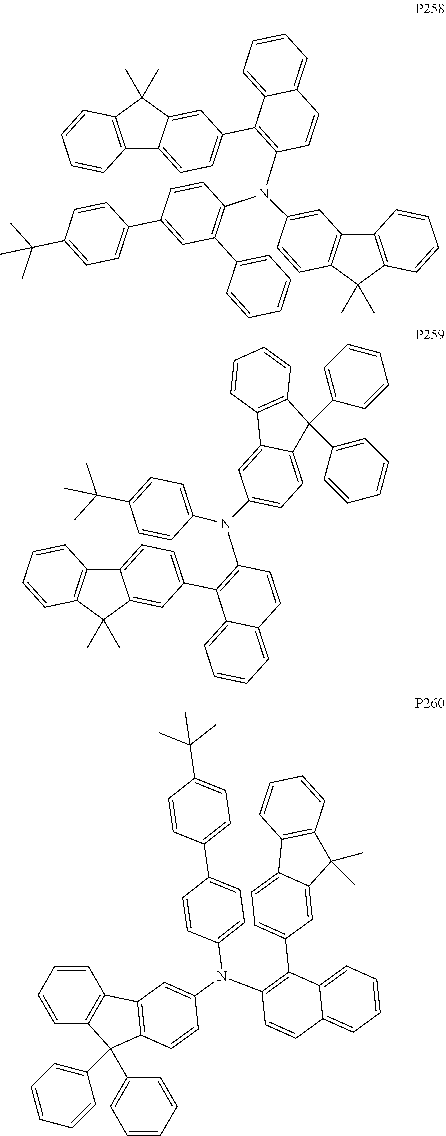

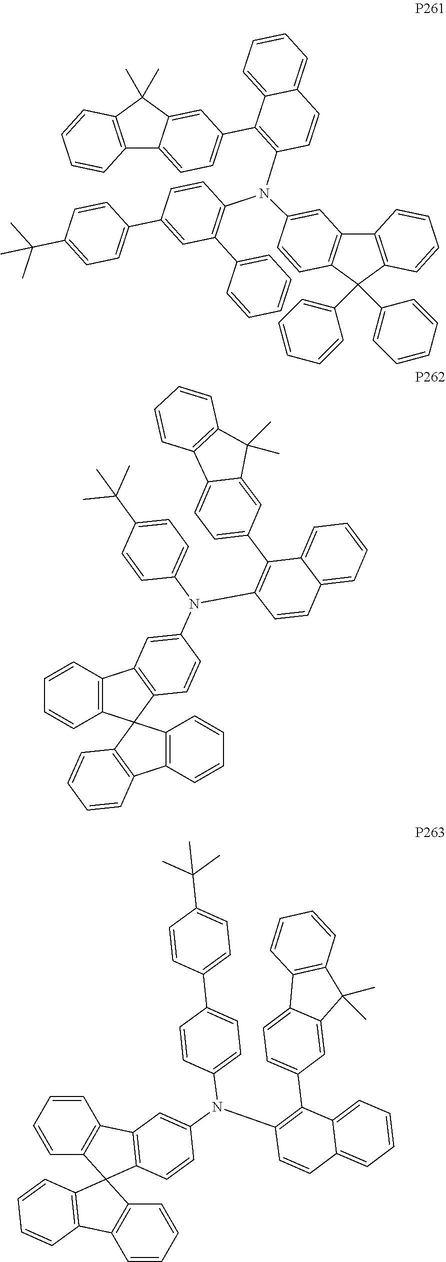 [SMILES: CC(C)(C)c1ccc(-c2ccc(N(c3ccc4c(c3)-c3ccccc3C4(c3ccccc3)c3ccccc3)c3ccc4ccccc4c3-c3ccc4c(c3)C(C)(C)c3ccccc3-4)c(-c3ccccc3)c2)cc1 ; CC(C)(C)c1ccc(N(c2ccc3c(c2)-c2ccccc2C32c3ccccc3-c3ccccc32)c2ccc3ccccc3c2-c2ccc3c(c2)C(C)(C)c2ccccc2-3)cc1 ; CC(C)(C)c1ccc(-c2ccc(N(c3ccc4c(c3)-c3ccccc3C43c4ccccc4-c4ccccc43)c3ccc4ccccc4c3-c3ccc4c(c3)C(C)(C)c3ccccc3-4)cc2)cc1]

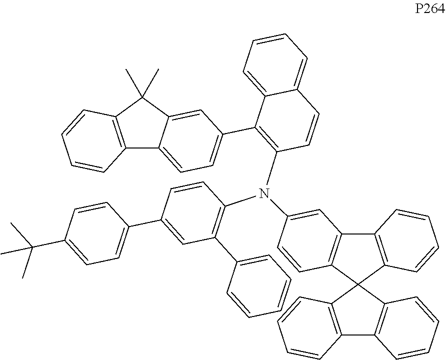 [SMILES: CC(C)(C)c1ccc(-c2ccc(N(c3ccc4c(c3)-c3ccccc3C43c4ccccc4-c4ccccc43)c3ccc4ccccc4c3-c3ccc4c(c3)C(C)(C)c3ccccc3-4)c(-c3ccccc3)c2)cc1]

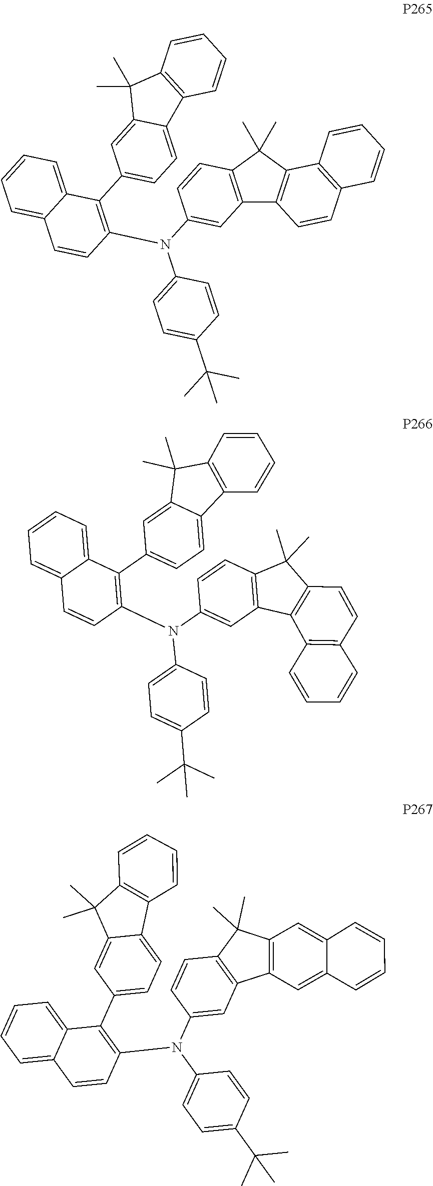

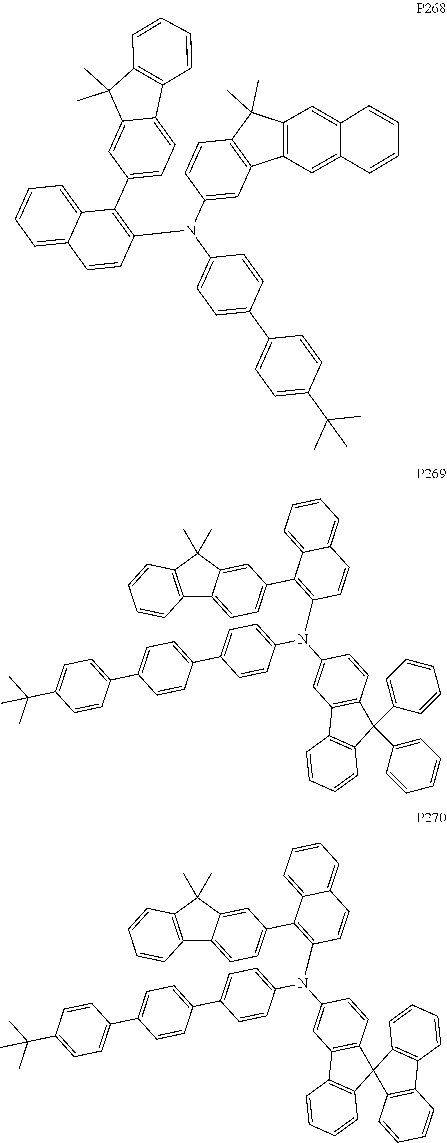 [SMILES: CC(C)(C)c1ccc(-c2ccc(N(c3ccc4c(c3)-c3cc5ccccc5cc3C4(C)C)c3ccc4ccccc4c3-c3ccc4c(c3)C(C)(C)c3ccccc3-4)cc2)cc1 ; CC(C)(C)c1ccc(-c2ccc(-c3ccc(N(c4ccc5c(c4)-c4ccccc4C5(c4ccccc4)c4ccccc4)c4ccc5ccccc5c4-c4ccc5c(c4)C(C)(C)c4ccccc4-5)cc3)cc2)cc1 ; CC(C)(C)c1ccc(-c2ccc(-c3ccc(N(c4ccc5c(c4)-c4ccccc4C54c5ccccc5-c5ccccc54)c4ccc5ccccc5c4-c4ccc5c(c4)C(C)(C)c4ccccc4-5)cc3)cc2)cc1]

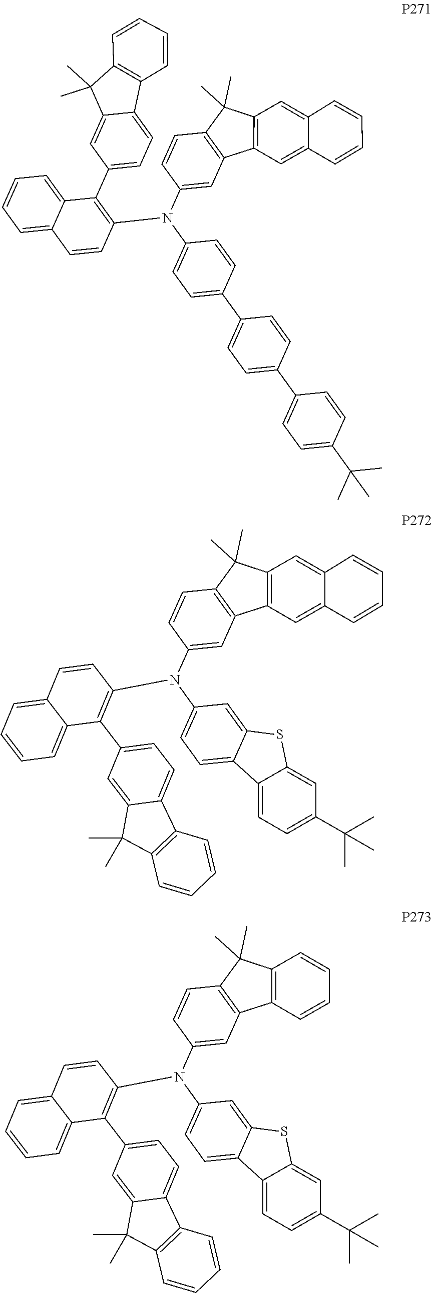 [SMILES: CC(C)(C)c1ccc(-c2ccc(-c3ccc(N(c4ccc5c(c4)-c4cc6ccccc6cc4C5(C)C)c4ccc5ccccc5c4-c4ccc5c(c4)C(C)(C)c4ccccc4-5)cc3)cc2)cc1 ; CC(C)(C)c1ccc2c(c1)sc1cc(N(c3ccc4c(c3)-c3cc5ccccc5cc3C4(C)C)c3ccc4ccccc4c3-c3ccc4c(c3)C(C)(C)c3ccccc3-4)ccc12 ; CC(C)(C)c1ccc2c(c1)sc1cc(N(c3ccc4c(c3)-c3ccccc3C4(C)C)c3ccc4ccccc4c3-c3ccc4c(c3)C(C)(C)c3ccccc3-4)ccc12]

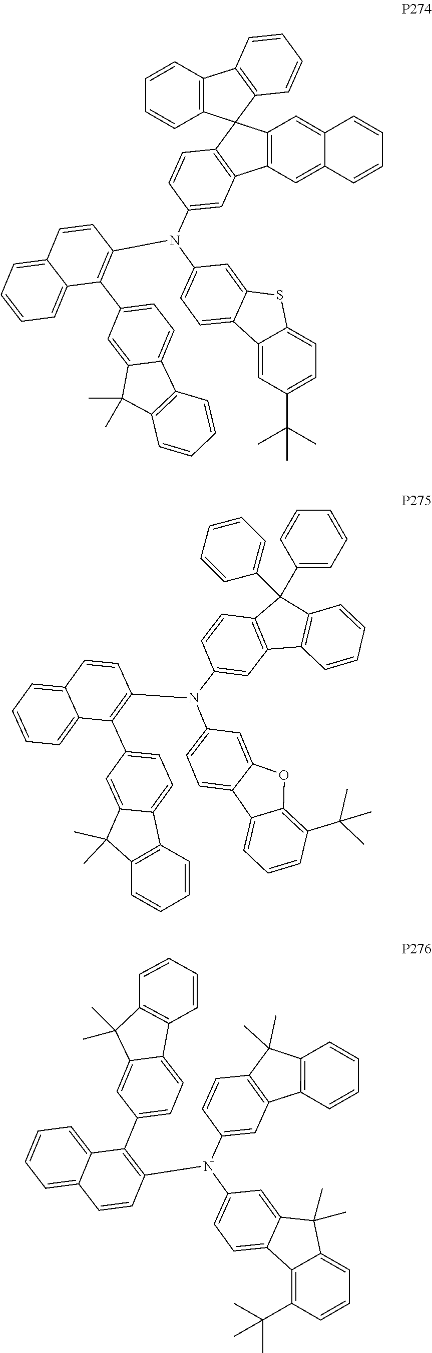 [SMILES: CC(C)(C)c1ccc2sc3cc(N(c4ccc5c(c4)-c4cc6ccccc6cc4C54c5ccccc5-c5ccccc54)c4ccc5ccccc5c4-c4ccc5c(c4)C(C)(C)c4ccccc4-5)ccc3c2c1 ; CC(C)(C)c1cccc2c1oc1cc(N(c3ccc4c(c3)-c3ccccc3C4(c3ccccc3)c3ccccc3)c3ccc4ccccc4c3-c3ccc4c(c3)C(C)(C)c3ccccc3-4)ccc12 ; CC(C)(C)c1cccc2c1-c1ccc(N(c3ccc4c(c3)-c3ccccc3C4(C)C)c3ccc4ccccc4c3-c3ccc4c(c3)C(C)(C)c3ccccc3-4)cc1C2(C)C]

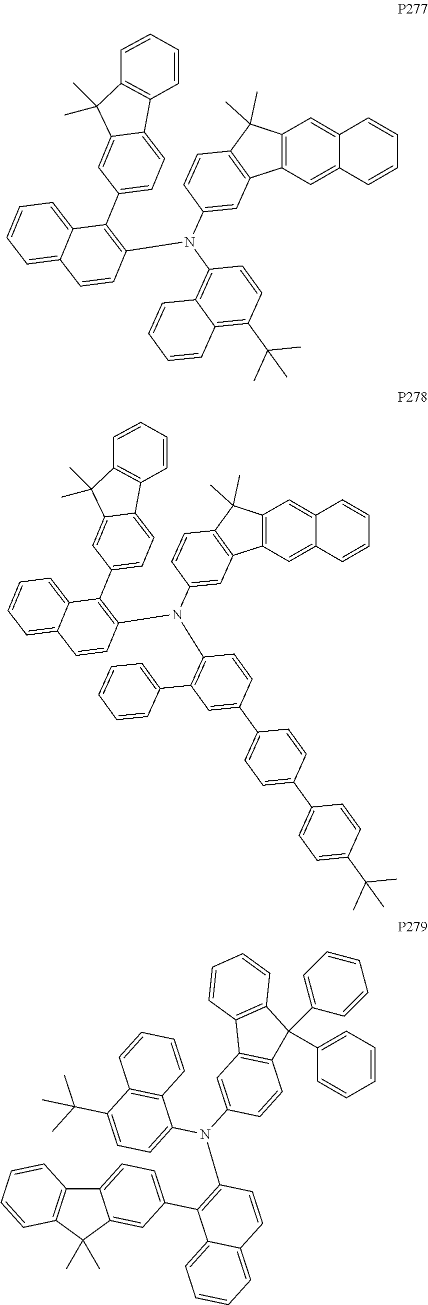 [SMILES: CC(C)(C)c1ccc(N(c2ccc3c(c2)-c2cc4ccccc4cc2C3(C)C)c2ccc3ccccc3c2-c2ccc3c(c2)C(C)(C)c2ccccc2-3)c2ccccc12 ; CC(C)(C)c1ccc(-c2ccc(-c3ccc(N(c4ccc5c(c4)-c4cc6ccccc6cc4C5(C)C)c4ccc5ccccc5c4-c4ccc5c(c4)C(C)(C)c4ccccc4-5)c(-c4ccccc4)c3)cc2)cc1 ; CC(C)(C)c1ccc(N(c2ccc3c(c2)-c2ccccc2C3(c2ccccc2)c2ccccc2)c2ccc3ccccc3c2-c2ccc3c(c2)C(C)(C)c2ccccc2-3)c2ccccc12]

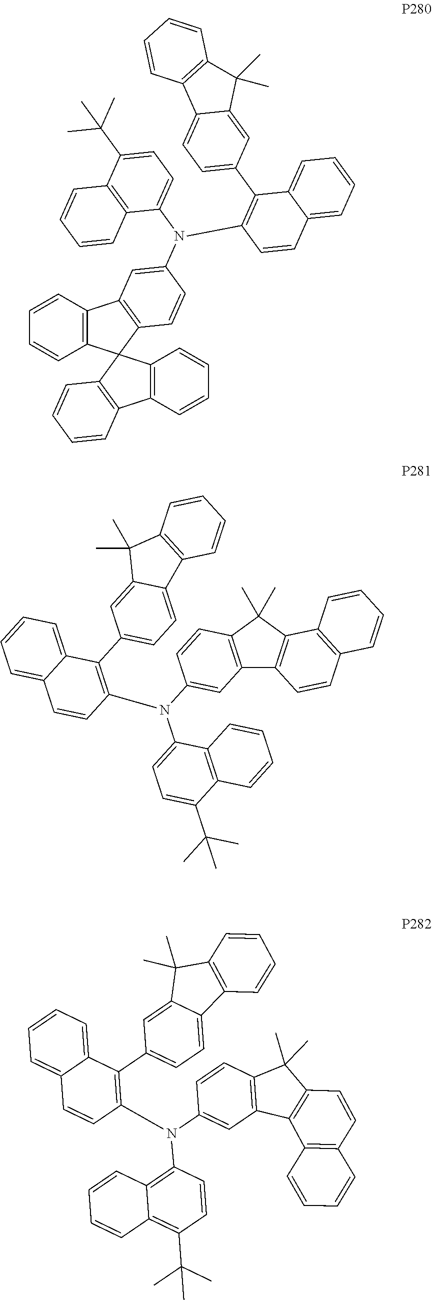 [SMILES: CC(C)(C)c1ccc(N(c2ccc3c(c2)-c2ccccc2C32c3ccccc3-c3ccccc32)c2ccc3ccccc3c2-c2ccc3c(c2)C(C)(C)c2ccccc2-3)c2ccccc12 ; CC(C)(C)c1ccc(N(c2ccc3c(c2)-c2ccc4ccccc4c2C3(C)C)c2ccc3ccccc3c2-c2ccc3c(c2)C(C)(C)c2ccccc2-3)c2ccccc12 ; CC(C)(C)c1ccc(N(c2ccc3c(c2)-c2c(ccc4ccccc24)C3(C)C)c2ccc3ccccc3c2-c2ccc3c(c2)C(C)(C)c2ccccc2-3)c2ccccc12]

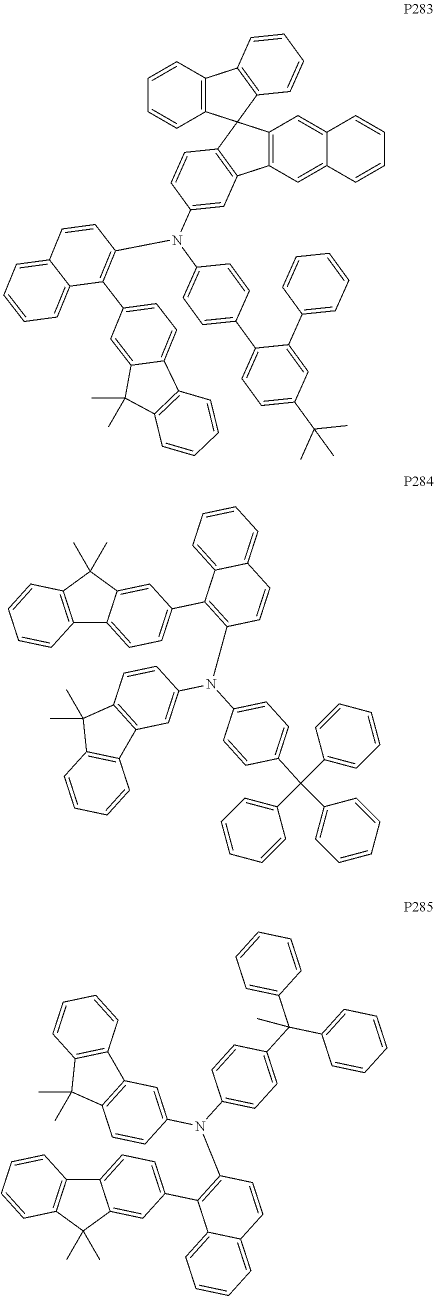 [SMILES: CC(C)(C)c1ccc(-c2ccc(N(c3ccc4c(c3)-c3cc5ccccc5cc3C43c4ccccc4-c4ccccc43)c3ccc4ccccc4c3-c3ccc4c(c3)C(C)(C)c3ccccc3-4)cc2)c(-c2ccccc2)c1 ; CC1(C)c2ccccc2-c2cc(N(c3ccc(C(c4ccccc4)(c4ccccc4)c4ccccc4)cc3)c3ccc4ccccc4c3-c3ccc4c(c3)C(C)(C)c3ccccc3-4)ccc21 ; CC1(C)c2ccccc2-c2cc(N(c3ccc(C(C)(c4ccccc4)c4ccccc4)cc3)c3ccc4ccccc4c3-c3ccc4c(c3)C(C)(C)c3ccccc3-4)ccc21]

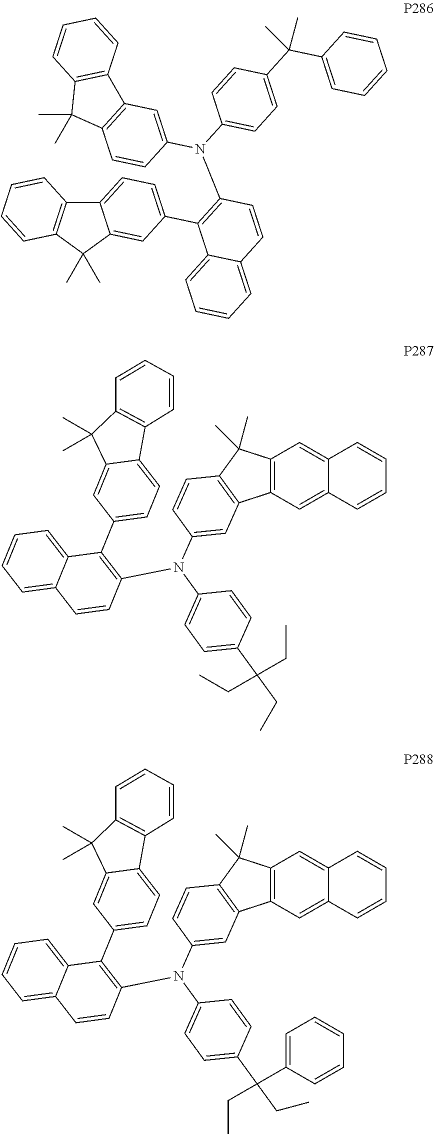 [SMILES: CC(C)(c1ccccc1)c1ccc(N(c2ccc3c(c2)-c2ccccc2C3(C)C)c2ccc3ccccc3c2-c2ccc3c(c2)C(C)(C)c2ccccc2-3)cc1 ; CCC(CC)(CC)c1ccc(N(c2ccc3c(c2)-c2cc4ccccc4cc2C3(C)C)c2ccc3ccccc3c2-c2ccc3c(c2)C(C)(C)c2ccccc2-3)cc1 ; CCC(CC)(c1ccccc1)c1ccc(N(c2ccc3c(c2)-c2cc4ccccc4cc2C3(C)C)c2ccc3ccccc3c2-c2ccc3c(c2)C(C)(C)c2ccccc2-3)cc1]

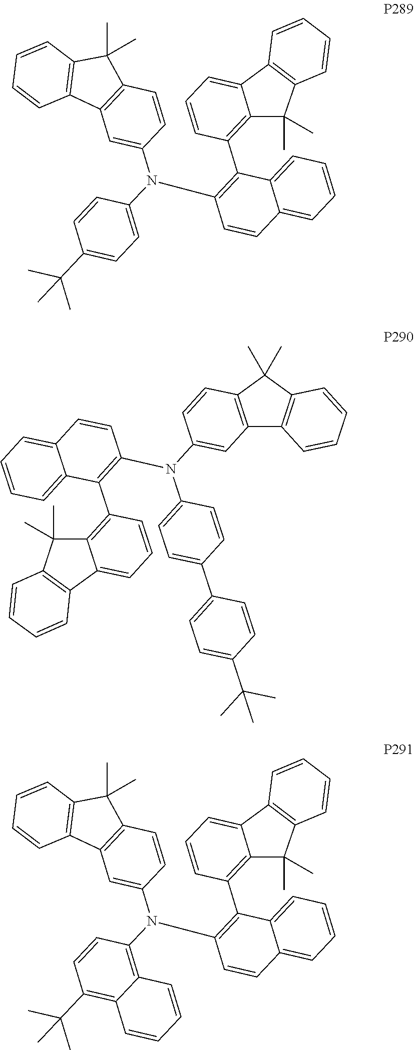 [SMILES: CC(C)(C)c1ccc(N(c2ccc3c(c2)-c2ccccc2C3(C)C)c2ccc3ccccc3c2-c2cccc3c2C(C)(C)c2ccccc2-3)cc1 ; CC(C)(C)c1ccc(-c2ccc(N(c3ccc4c(c3)-c3ccccc3C4(C)C)c3ccc4ccccc4c3-c3cccc4c3C(C)(C)c3ccccc3-4)cc2)cc1 ; CC(C)(C)c1ccc(N(c2ccc3c(c2)-c2ccccc2C3(C)C)c2ccc3ccccc3c2-c2cccc3c2C(C)(C)c2ccccc2-3)c2ccccc12]

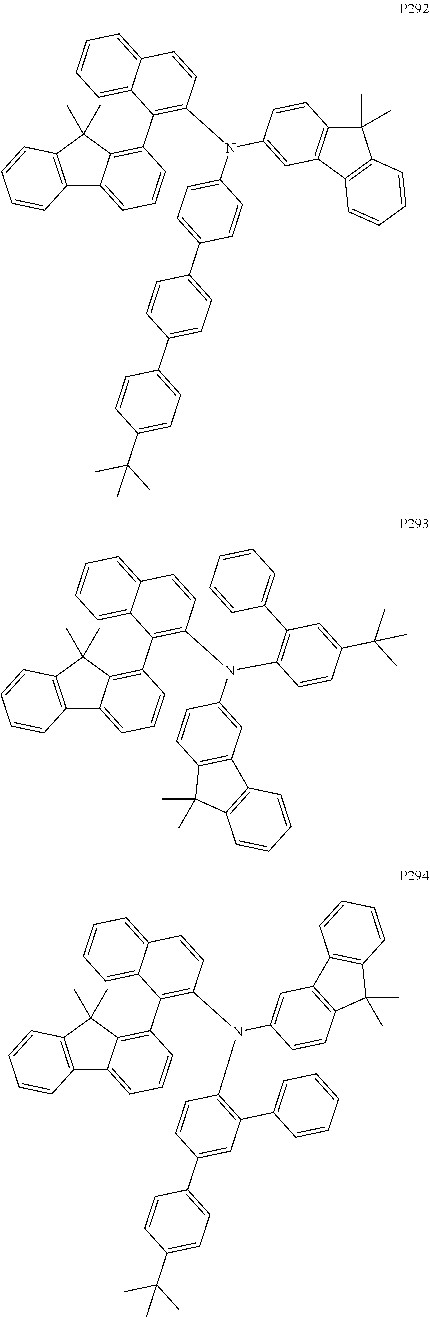 [SMILES: CC(C)(C)c1ccc(-c2ccc(-c3ccc(N(c4ccc5c(c4)-c4ccccc4C5(C)C)c4ccc5ccccc5c4-c4cccc5c4C(C)(C)c4ccccc4-5)cc3)cc2)cc1 ; CC(C)(C)c1ccc(N(c2ccc3c(c2)-c2ccccc2C3(C)C)c2ccc3ccccc3c2-c2cccc3c2C(C)(C)c2ccccc2-3)c(-c2ccccc2)c1 ; CC(C)(C)c1ccc(-c2ccc(N(c3ccc4c(c3)-c3ccccc3C4(C)C)c3ccc4ccccc4c3-c3cccc4c3C(C)(C)c3ccccc3-4)c(-c3ccccc3)c2)cc1]

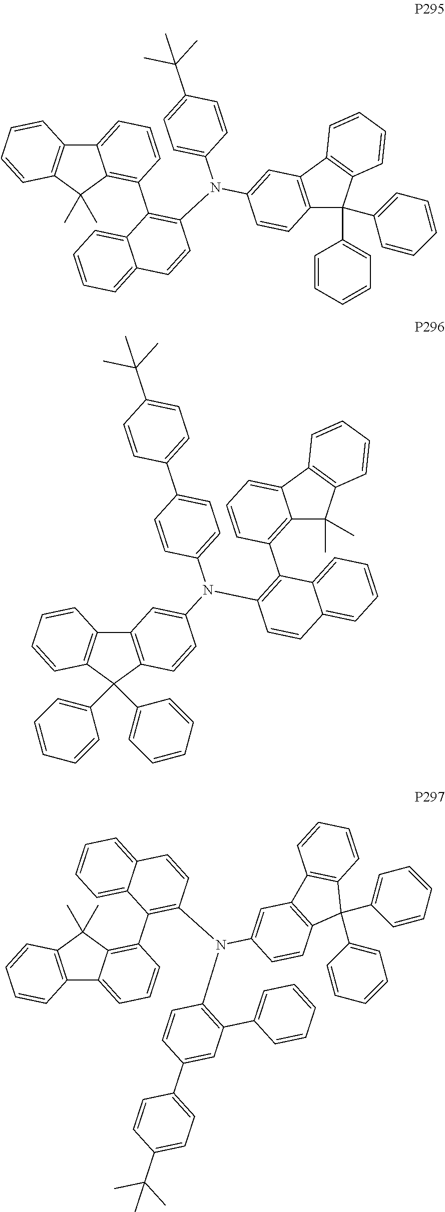 [SMILES: CC(C)(C)c1ccc(N(c2ccc3c(c2)-c2ccccc2C3(c2ccccc2)c2ccccc2)c2ccc3ccccc3c2-c2cccc3c2C(C)(C)c2ccccc2-3)cc1 ; CC(C)(C)c1ccc(-c2ccc(N(c3ccc4c(c3)-c3ccccc3C4(c3ccccc3)c3ccccc3)c3ccc4ccccc4c3-c3cccc4c3C(C)(C)c3ccccc3-4)cc2)cc1 ; CC(C)(C)c1ccc(-c2ccc(N(c3ccc4c(c3)-c3ccccc3C4(c3ccccc3)c3ccccc3)c3ccc4ccccc4c3-c3cccc4c3C(C)(C)c3ccccc3-4)c(-c3ccccc3)c2)cc1]

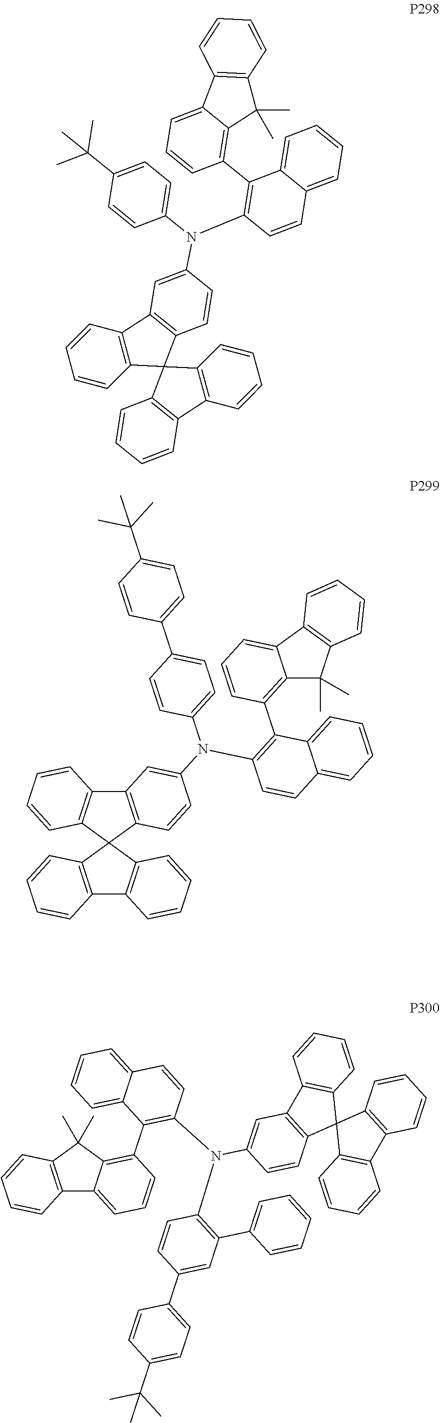

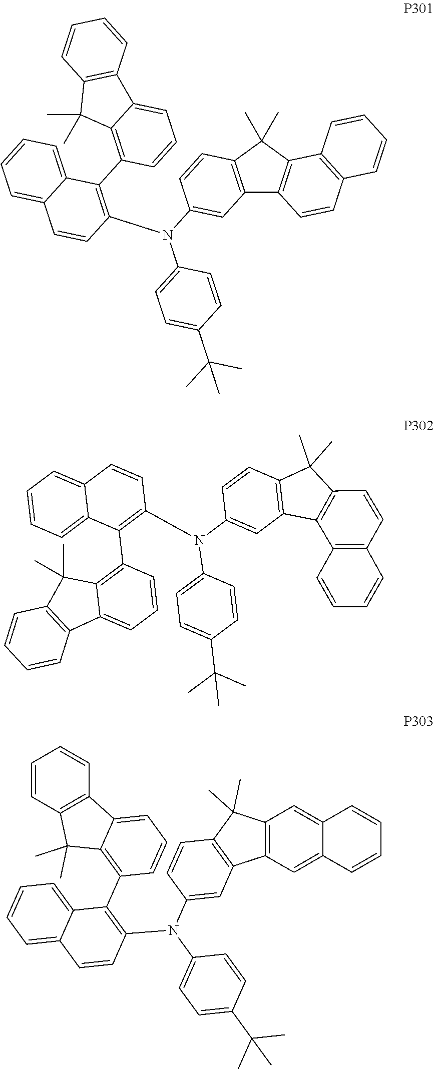 [SMILES: CC(C)(C)c1ccc(N(c2ccc3c(c2)-c2ccc4ccccc4c2C3(C)C)c2ccc3ccccc3c2-c2cccc3c2C(C)(C)c2ccccc2-3)cc1 ; CC(C)(C)c1ccc(N(c2ccc3c(c2)-c2c(ccc4ccccc24)C3(C)C)c2ccc3ccccc3c2-c2cccc3c2C(C)(C)c2ccccc2-3)cc1 ; CC(C)(C)c1ccc(N(c2ccc3c(c2)-c2cc4ccccc4cc2C3(C)C)c2ccc3ccccc3c2-c2cccc3c2C(C)(C)c2ccccc2-3)cc1]

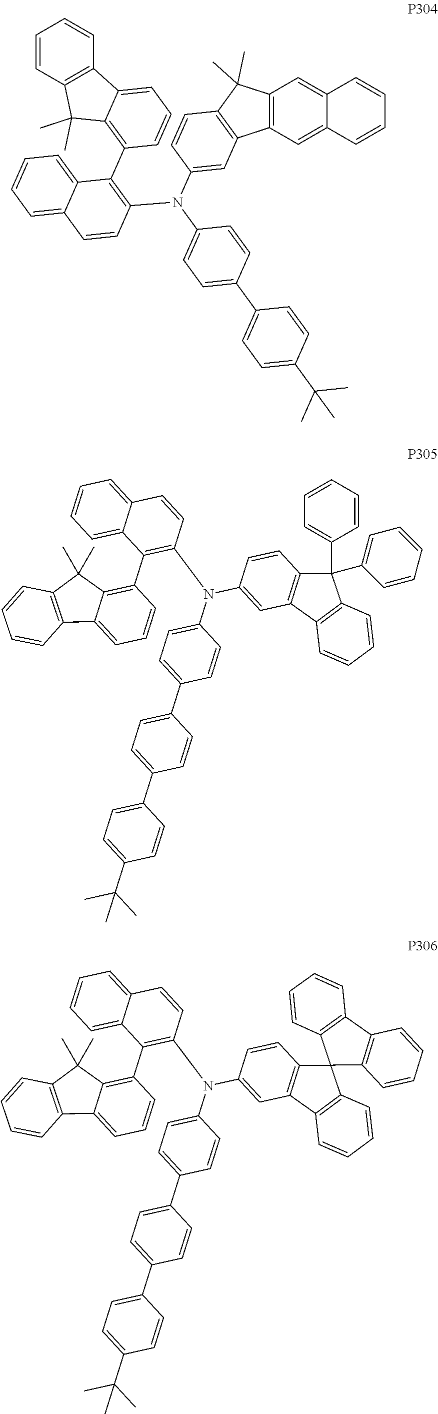 [SMILES: CC(C)(C)c1ccc(-c2ccc(N(c3ccc4c(c3)-c3cc5ccccc5cc3C4(C)C)c3ccc4ccccc4c3-c3cccc4c3C(C)(C)c3ccccc3-4)cc2)cc1 ; CC(C)(C)c1ccc(-c2ccc(-c3ccc(N(c4ccc5c(c4)-c4ccccc4C5(c4ccccc4)c4ccccc4)c4ccc5ccccc5c4-c4cccc5c4C(C)(C)c4ccccc4-5)cc3)cc2)cc1 ; CC(C)(C)c1ccc(-c2ccc(-c3ccc(N(c4ccc5c(c4)-c4ccccc4C54c5ccccc5-c5ccccc54)c4ccc5ccccc5c4-c4cccc5c4C(C)(C)c4ccccc4-5)cc3)cc2)cc1]

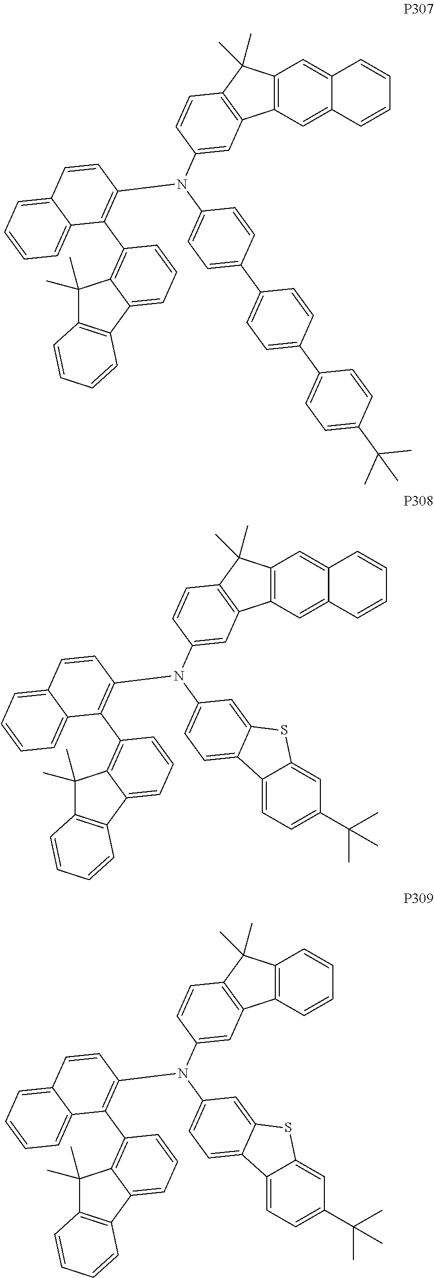 [SMILES: CC(C)(C)c1ccc(-c2ccc(-c3ccc(N(c4ccc5c(c4)-c4cc6ccccc6cc4C5(C)C)c4ccc5ccccc5c4-c4cccc5c4C(C)(C)c4ccccc4-5)cc3)cc2)cc1 ; CC(C)(C)c1ccc2c(c1)sc1cc(N(c3ccc4c(c3)-c3cc5ccccc5cc3C4(C)C)c3ccc4ccccc4c3-c3cccc4c3C(C)(C)c3ccccc3-4)ccc12 ; CC(C)(C)c1ccc2c(c1)sc1cc(N(c3ccc4c(c3)-c3ccccc3C4(C)C)c3ccc4ccccc4c3-c3cccc4c3C(C)(C)c3ccccc3-4)ccc12]

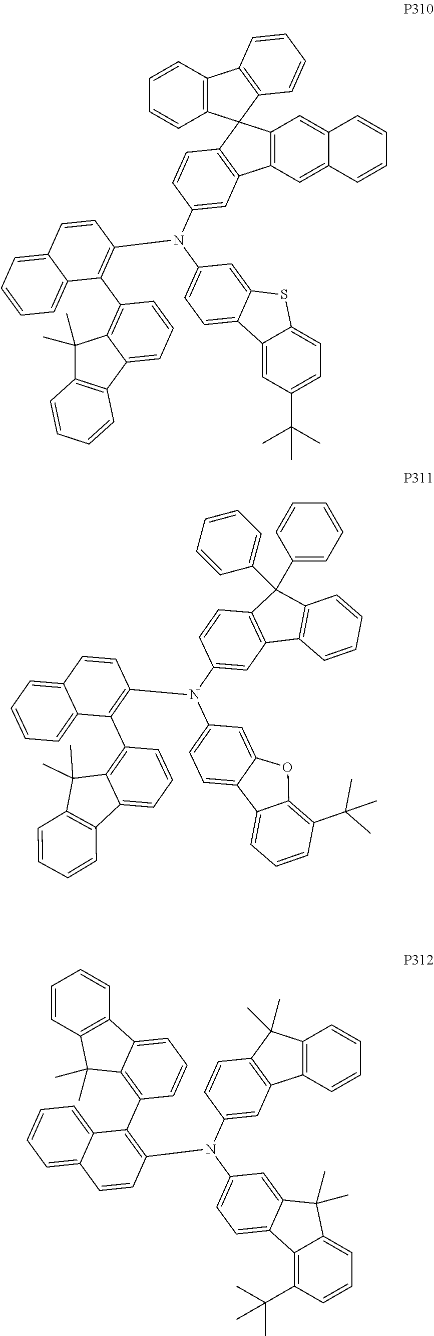

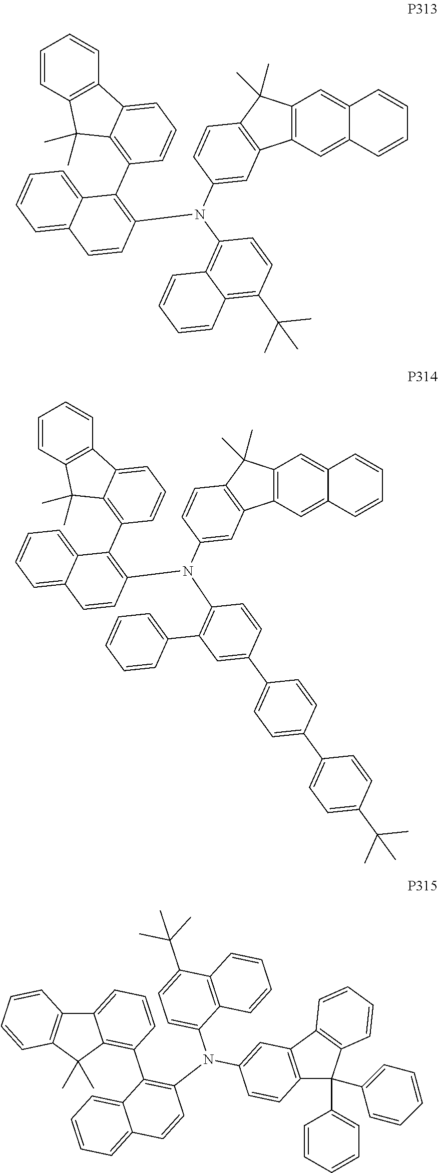 [SMILES: CC(C)(C)c1ccc(N(c2ccc3c(c2)-c2cc4ccccc4cc2C3(C)C)c2ccc3ccccc3c2-c2cccc3c2C(C)(C)c2ccccc2-3)c2ccccc12 ; CC(C)(C)c1ccc(-c2ccc(-c3ccc(N(c4ccc5c(c4)-c4cc6ccccc6cc4C5(C)C)c4ccc5ccccc5c4-c4cccc5c4C(C)(C)c4ccccc4-5)c(-c4ccccc4)c3)cc2)cc1 ; CC(C)(C)c1ccc(N(c2ccc3c(c2)-c2ccccc2C3(c2ccccc2)c2ccccc2)c2ccc3ccccc3c2-c2cccc3c2C(C)(C)c2ccccc2-3)c2ccccc12]

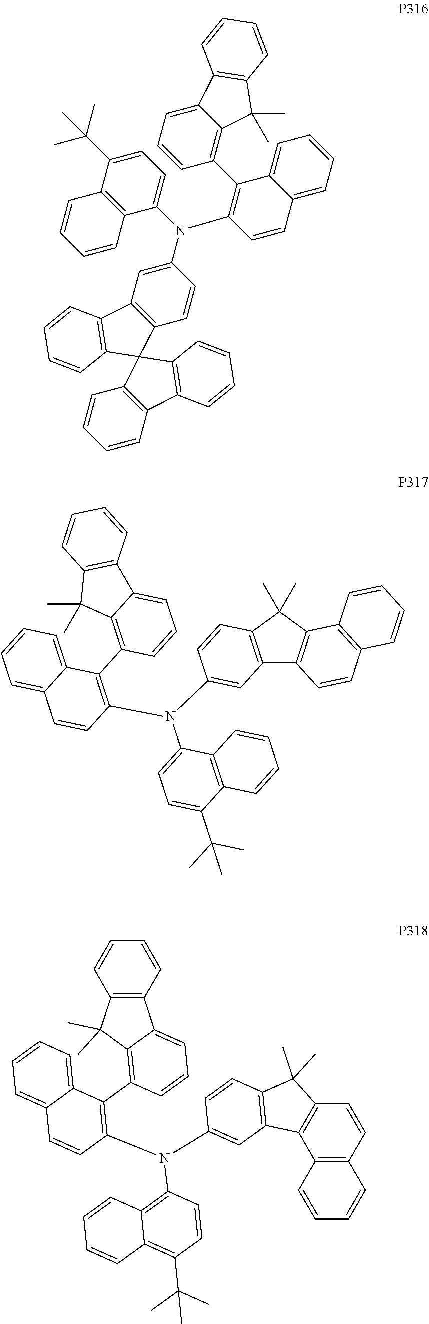 [SMILES: CC(C)(C)c1ccc(N(c2ccc3c(c2)-c2ccccc2C32c3ccccc3-c3ccccc32)c2ccc3ccccc3c2-c2cccc3c2C(C)(C)c2ccccc2-3)c2ccccc12 ; CC(C)(C)c1ccc(N(c2ccc3c(c2)-c2ccc4ccccc4c2C3(C)C)c2ccc3ccccc3c2-c2cccc3c2C(C)(C)c2ccccc2-3)c2ccccc12 ; CC(C)(C)c1ccc(N(c2ccc3c(c2)-c2c(ccc4ccccc24)C3(C)C)c2ccc3ccccc3c2-c2cccc3c2C(C)(C)c2ccccc2-3)c2ccccc12]

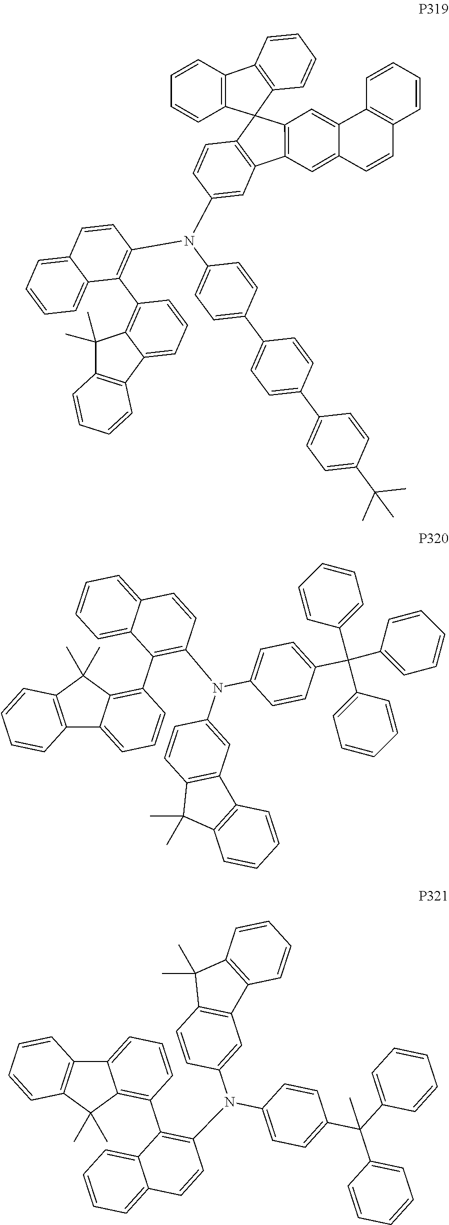 [SMILES: CC(C)(C)c1ccc(-c2ccc(-c3ccc(N(c4ccc5c(c4)-c4cc6ccc7ccccc7c6cc4C54c5ccccc5-c5ccccc54)c4ccc5ccccc5c4-c4cccc5c4C(C)(C)c4ccccc4-5)cc3)cc2)cc1 ; CC1(C)c2ccccc2-c2cc(N(c3ccc(C(c4ccccc4)(c4ccccc4)c4ccccc4)cc3)c3ccc4ccccc4c3-c3cccc4c3C(C)(C)c3ccccc3-4)ccc21 ; CC1(C)c2ccccc2-c2cc(N(c3ccc(C(C)(c4ccccc4)c4ccccc4)cc3)c3ccc4ccccc4c3-c3cccc4c3C(C)(C)c3ccccc3-4)ccc21]

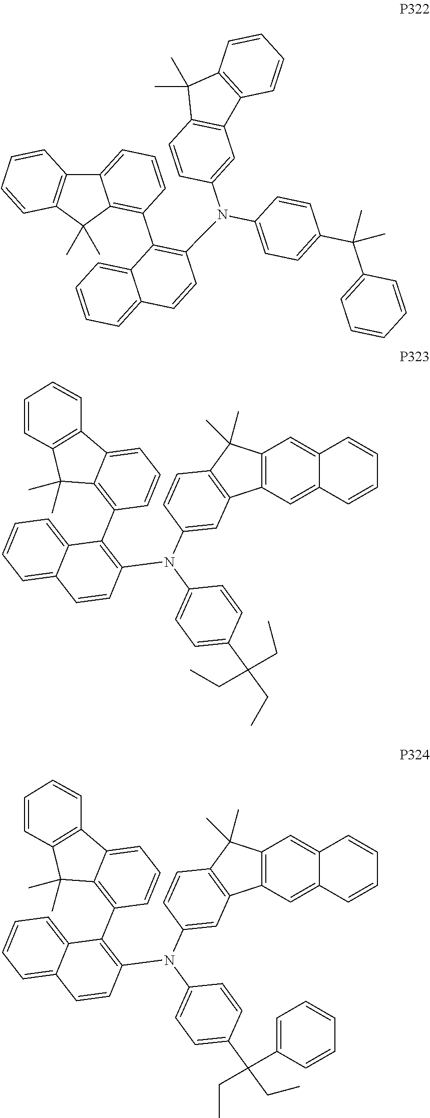 [SMILES: CC(C)(c1ccccc1)c1ccc(N(c2ccc3c(c2)-c2ccccc2C3(C)C)c2ccc3ccccc3c2-c2cccc3c2C(C)(C)c2ccccc2-3)cc1 ; CCC(CC)(CC)c1ccc(N(c2ccc3c(c2)-c2cc4ccccc4cc2C3(C)C)c2ccc3ccccc3c2-c2cccc3c2C(C)(C)c2ccccc2-3)cc1 ; CCC(CC)(c1ccccc1)c1ccc(N(c2ccc3c(c2)-c2cc4ccccc4cc2C3(C)C)c2ccc3ccccc3c2-c2cccc3c2C(C)(C)c2ccccc2-3)cc1]

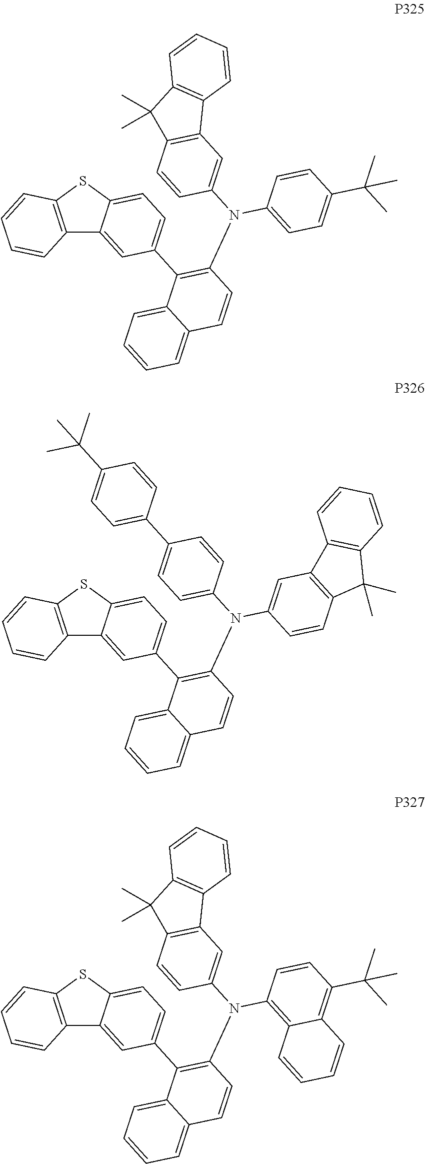 [SMILES: CC(C)(C)c1ccc(N(c2ccc3c(c2)-c2ccccc2C3(C)C)c2ccc3ccccc3c2-c2ccc3sc4ccccc4c3c2)cc1 ; CC(C)(C)c1ccc(-c2ccc(N(c3ccc4c(c3)-c3ccccc3C4(C)C)c3ccc4ccccc4c3-c3ccc4sc5ccccc5c4c3)cc2)cc1 ; CC(C)(C)c1ccc(N(c2ccc3c(c2)-c2ccccc2C3(C)C)c2ccc3ccccc3c2-c2ccc3sc4ccccc4c3c2)c2ccccc12]

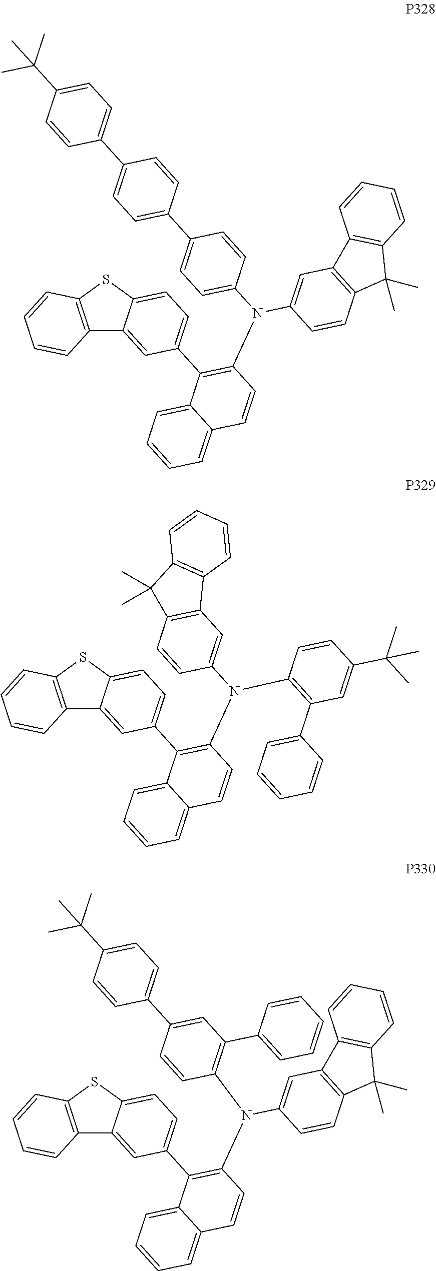 [SMILES: CC(C)(C)c1ccc(-c2ccc(-c3ccc(N(c4ccc5c(c4)-c4ccccc4C5(C)C)c4ccc5ccccc5c4-c4ccc5sc6ccccc6c5c4)cc3)cc2)cc1 ; CC(C)(C)c1ccc(N(c2ccc3c(c2)-c2ccccc2C3(C)C)c2ccc3ccccc3c2-c2ccc3sc4ccccc4c3c2)c(-c2ccccc2)c1 ; CC(C)(C)c1ccc(-c2ccc(N(c3ccc4c(c3)-c3ccccc3C4(C)C)c3ccc4ccccc4c3-c3ccc4sc5ccccc5c4c3)c(-c3ccccc3)c2)cc1]

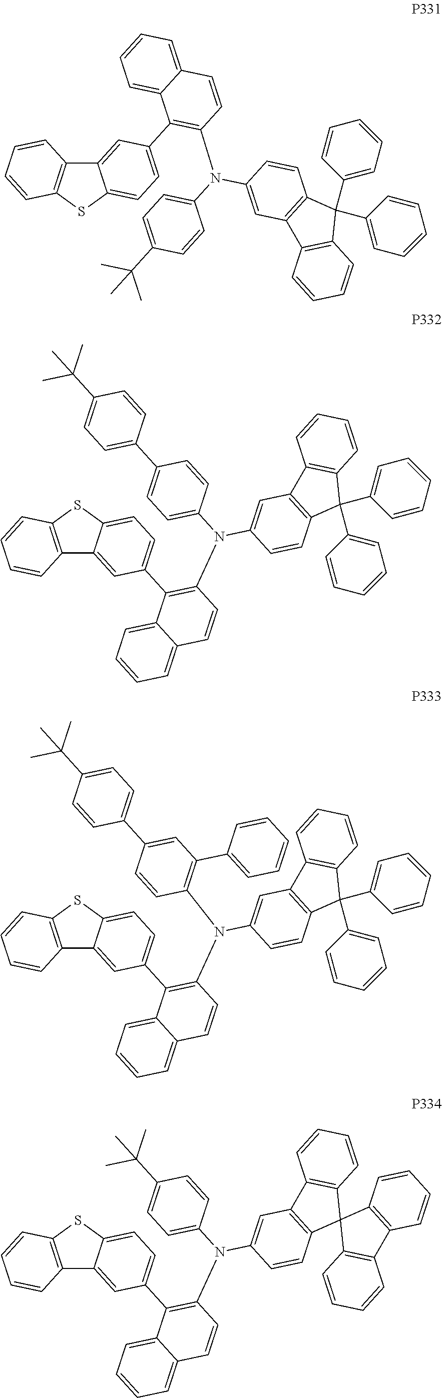 [SMILES: CC(C)(C)c1ccc(N(c2ccc3c(c2)-c2ccccc2C3(c2ccccc2)c2ccccc2)c2ccc3ccccc3c2-c2ccc3sc4ccccc4c3c2)cc1 ; CC(C)(C)c1ccc(-c2ccc(N(c3ccc4c(c3)-c3ccccc3C4(c3ccccc3)c3ccccc3)c3ccc4ccccc4c3-c3ccc4sc5ccccc5c4c3)cc2)cc1 ; CC(C)(C)c1ccc(-c2ccc(N(c3ccc4c(c3)-c3ccccc3C4(c3ccccc3)c3ccccc3)c3ccc4ccccc4c3-c3ccc4sc5ccccc5c4c3)c(-c3ccccc3)c2)cc1 ; CC(C)(C)c1ccc(N(c2ccc3c(c2)-c2ccccc2C32c3ccccc3-c3ccccc32)c2ccc3ccccc3c2-c2ccc3sc4ccccc4c3c2)cc1]

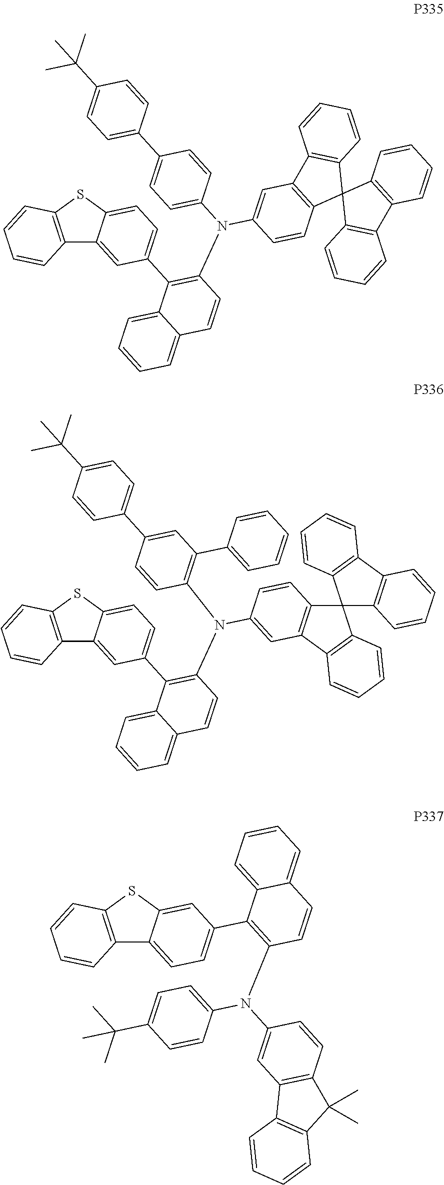 [SMILES: CC(C)(C)c1ccc(-c2ccc(N(c3ccc4c(c3)-c3ccccc3C43c4ccccc4-c4ccccc43)c3ccc4ccccc4c3-c3ccc4sc5ccccc5c4c3)cc2)cc1 ; CC(C)(C)c1ccc(-c2ccc(N(c3ccc4c(c3)-c3ccccc3C43c4ccccc4-c4ccccc43)c3ccc4ccccc4c3-c3ccc4sc5ccccc5c4c3)c(-c3ccccc3)c2)cc1 ; CC(C)(C)c1ccc(N(c2ccc3c(c2)-c2ccccc2C3(C)C)c2ccc3ccccc3c2-c2ccc3c(c2)sc2ccccc23)cc1]

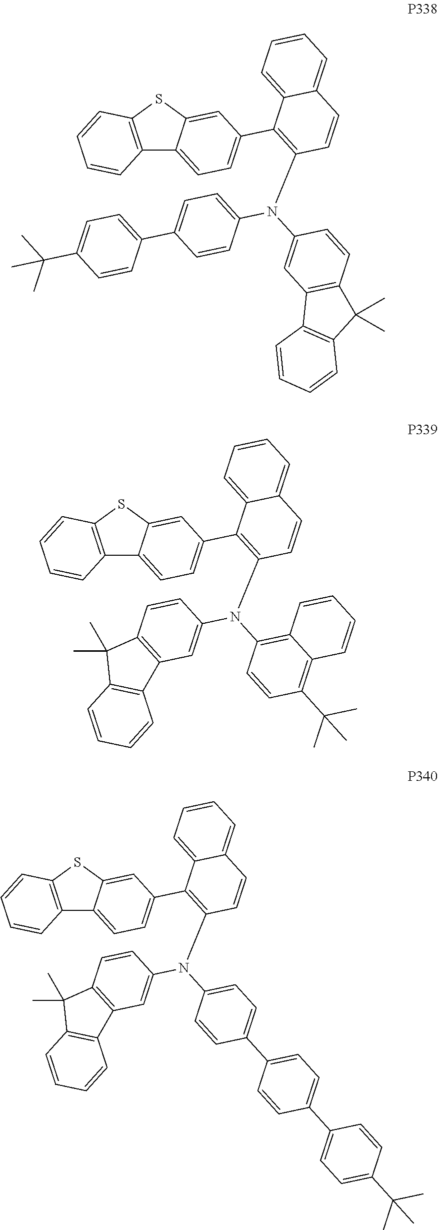 [SMILES: CC(C)(C)c1ccc(-c2ccc(N(c3ccc4c(c3)-c3ccccc3C4(C)C)c3ccc4ccccc4c3-c3ccc4c(c3)sc3ccccc34)cc2)cc1 ; CC(C)(C)c1ccc(N(c2ccc3c(c2)-c2ccccc2C3(C)C)c2ccc3ccccc3c2-c2ccc3c(c2)sc2ccccc23)c2ccccc12 ; CC(C)(C)c1ccc(-c2ccc(-c3ccc(N(c4ccc5c(c4)-c4ccccc4C5(C)C)c4ccc5ccccc5c4-c4ccc5c(c4)sc4ccccc45)cc3)cc2)cc1]

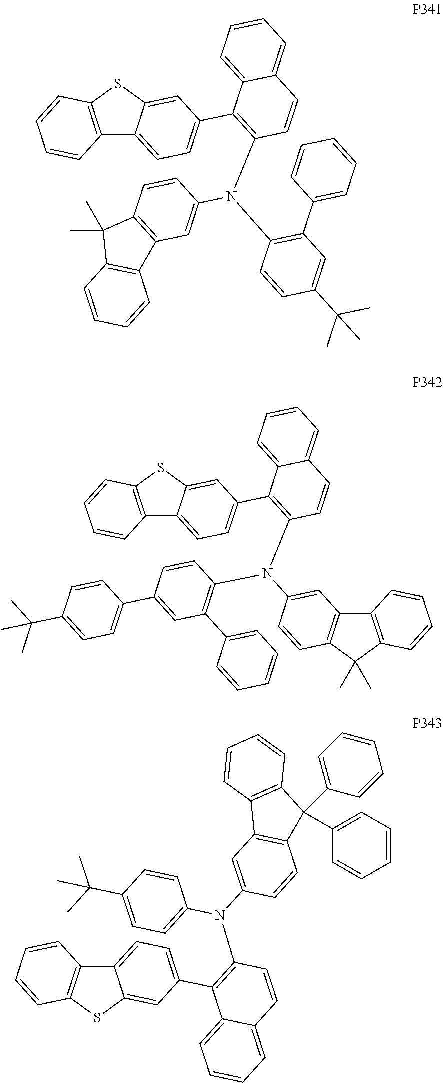

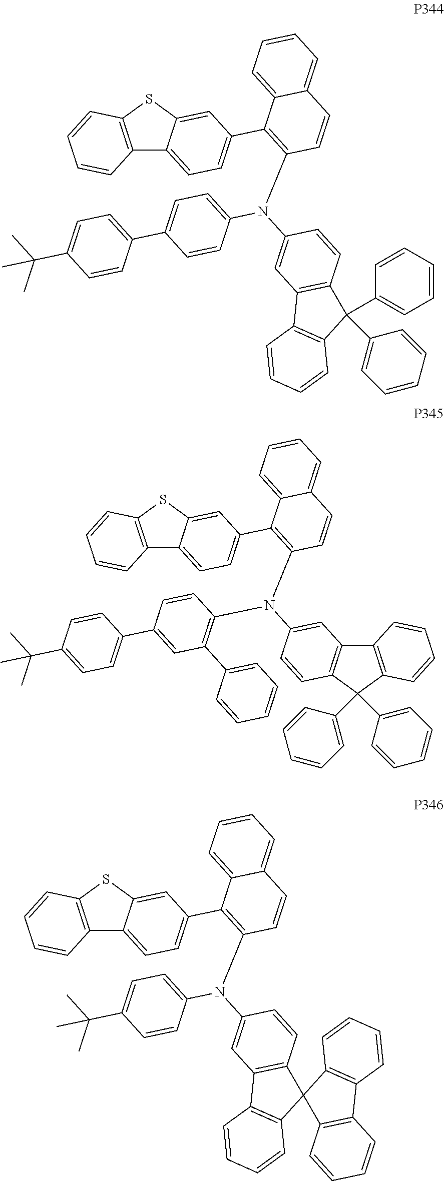 [SMILES: CC(C)(C)c1ccc(-c2ccc(N(c3ccc4c(c3)-c3ccccc3C4(c3ccccc3)c3ccccc3)c3ccc4ccccc4c3-c3ccc4c(c3)sc3ccccc34)cc2)cc1 ; CC(C)(C)c1ccc(-c2ccc(N(c3ccc4c(c3)-c3ccccc3C4(c3ccccc3)c3ccccc3)c3ccc4ccccc4c3-c3ccc4c(c3)sc3ccccc34)c(-c3ccccc3)c2)cc1 ; CC(C)(C)c1ccc(N(c2ccc3c(c2)-c2ccccc2C32c3ccccc3-c3ccccc32)c2ccc3ccccc3c2-c2ccc3c(c2)sc2ccccc23)cc1]

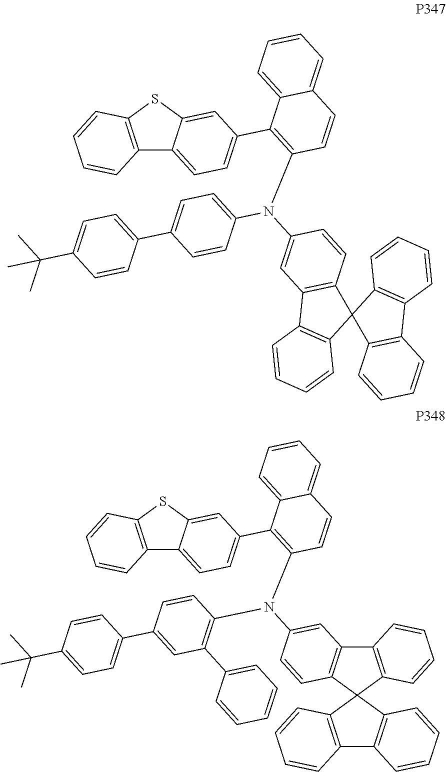 [SMILES: CC(C)(C)c1ccc(-c2ccc(N(c3ccc4c(c3)-c3ccccc3C43c4ccccc4-c4ccccc43)c3ccc4ccccc4c3-c3ccc4c(c3)sc3ccccc34)cc2)cc1 ; CC(C)(C)c1ccc(-c2ccc(N(c3ccc4c(c3)-c3ccccc3C43c4ccccc4-c4ccccc43)c3ccc4ccccc4c3-c3ccc4c(c3)sc3ccccc34)c(-c3ccccc3)c2)cc1]

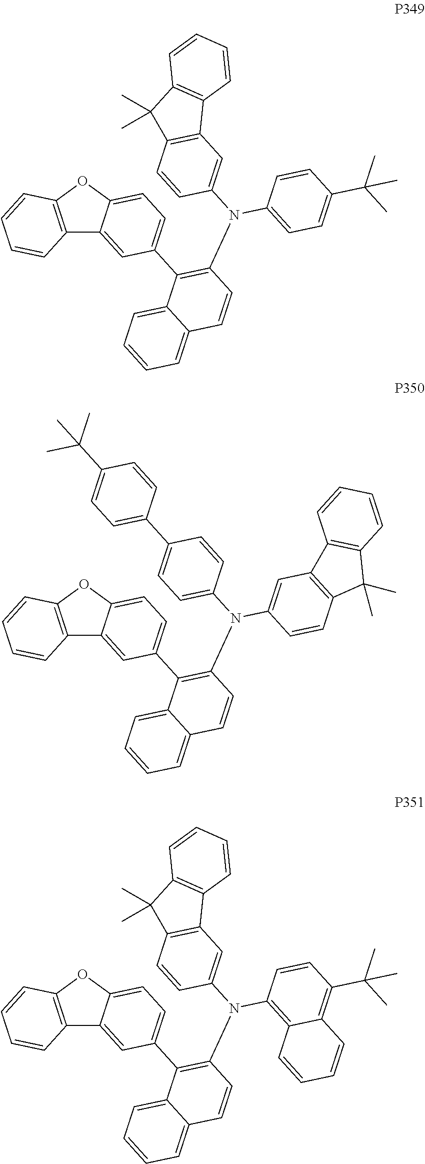 [SMILES: CC(C)(C)c1ccc(N(c2ccc3c(c2)-c2ccccc2C3(C)C)c2ccc3ccccc3c2-c2ccc3oc4ccccc4c3c2)cc1 ; CC(C)(C)c1ccc(-c2ccc(N(c3ccc4c(c3)-c3ccccc3C4(C)C)c3ccc4ccccc4c3-c3ccc4oc5ccccc5c4c3)cc2)cc1 ; CC(C)(C)c1ccc(N(c2ccc3c(c2)-c2ccccc2C3(C)C)c2ccc3ccccc3c2-c2ccc3oc4ccccc4c3c2)c2ccccc12]

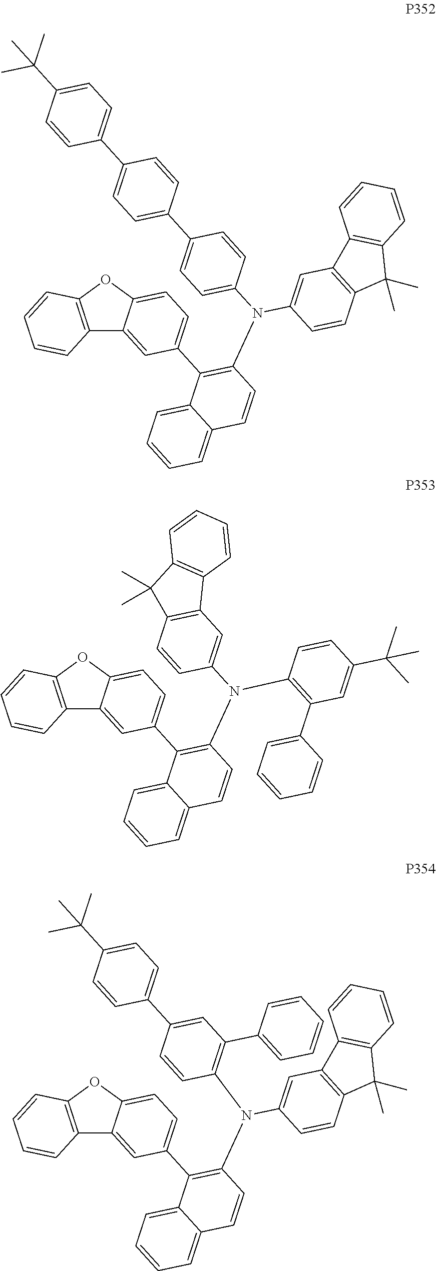 [SMILES: CC(C)(C)c1ccc(-c2ccc(-c3ccc(N(c4ccc5c(c4)-c4ccccc4C5(C)C)c4ccc5ccccc5c4-c4ccc5oc6ccccc6c5c4)cc3)cc2)cc1 ; CC(C)(C)c1ccc(N(c2ccc3c(c2)-c2ccccc2C3(C)C)c2ccc3ccccc3c2-c2ccc3oc4ccccc4c3c2)c(-c2ccccc2)c1 ; CC(C)(C)c1ccc(-c2ccc(N(c3ccc4c(c3)-c3ccccc3C4(C)C)c3ccc4ccccc4c3-c3ccc4oc5ccccc5c4c3)c(-c3ccccc3)c2)cc1]

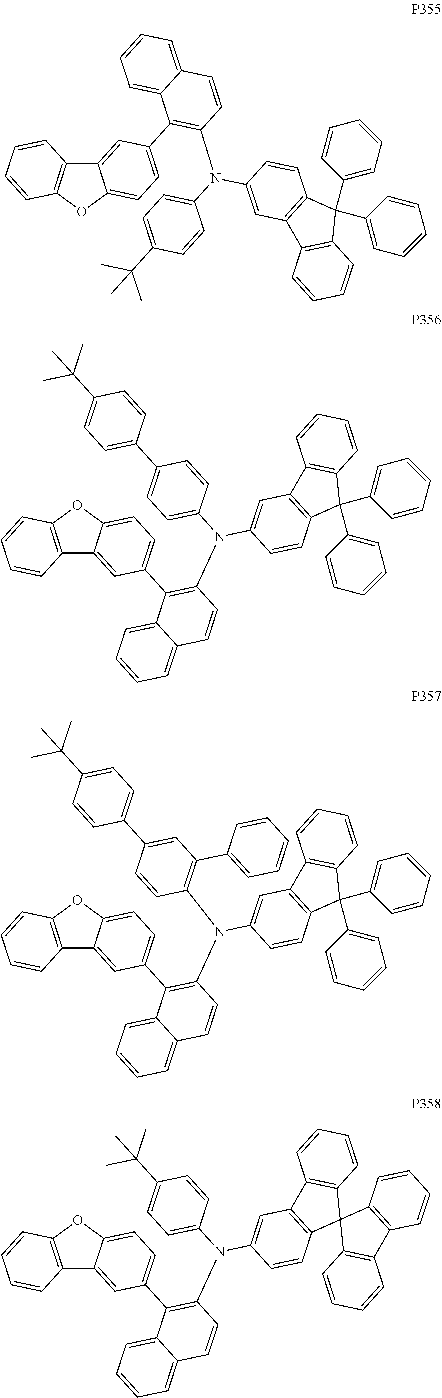 [SMILES: CC(C)(C)c1ccc(N(c2ccc3c(c2)-c2ccccc2C3(c2ccccc2)c2ccccc2)c2ccc3ccccc3c2-c2ccc3oc4ccccc4c3c2)cc1 ; CC(C)(C)c1ccc(-c2ccc(N(c3ccc4c(c3)-c3ccccc3C4(c3ccccc3)c3ccccc3)c3ccc4ccccc4c3-c3ccc4oc5ccccc5c4c3)cc2)cc1 ; CC(C)(C)c1ccc(-c2ccc(N(c3ccc4c(c3)-c3ccccc3C4(c3ccccc3)c3ccccc3)c3ccc4ccccc4c3-c3ccc4oc5ccccc5c4c3)c(-c3ccccc3)c2)cc1 ; CC(C)(C)c1ccc(N(c2ccc3c(c2)-c2ccccc2C32c3ccccc3-c3ccccc32)c2ccc3ccccc3c2-c2ccc3oc4ccccc4c3c2)cc1]

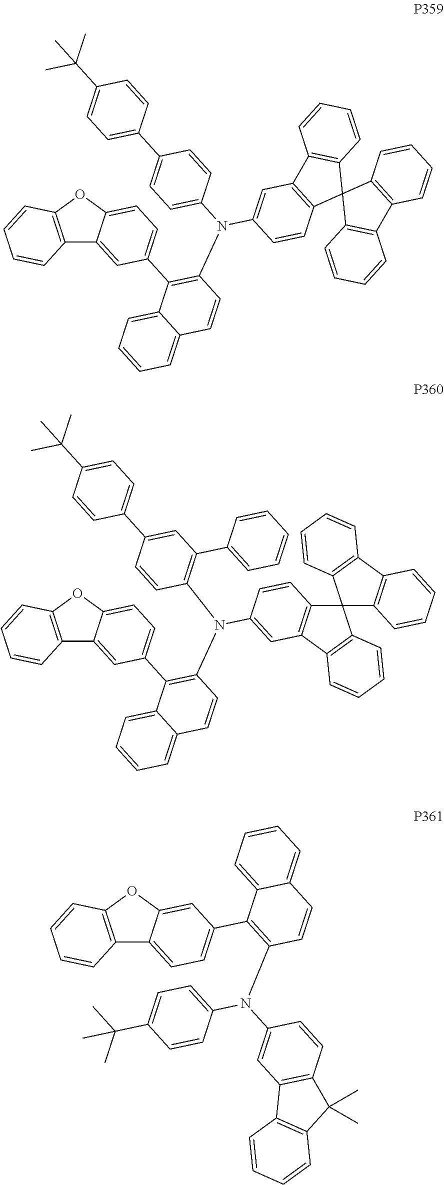 [SMILES: CC(C)(C)c1ccc(-c2ccc(N(c3ccc4c(c3)-c3ccccc3C43c4ccccc4-c4ccccc43)c3ccc4ccccc4c3-c3ccc4oc5ccccc5c4c3)cc2)cc1 ; CC(C)(C)c1ccc(-c2ccc(N(c3ccc4c(c3)-c3ccccc3C43c4ccccc4-c4ccccc43)c3ccc4ccccc4c3-c3ccc4oc5ccccc5c4c3)c(-c3ccccc3)c2)cc1 ; CC(C)(C)c1ccc(N(c2ccc3c(c2)-c2ccccc2C3(C)C)c2ccc3ccccc3c2-c2ccc3c(c2)oc2ccccc23)cc1]

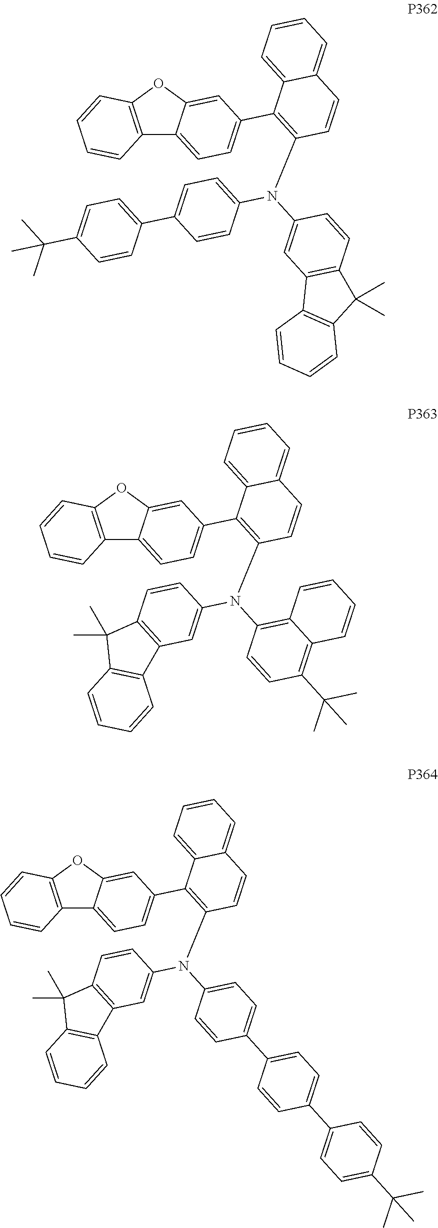 [SMILES: CC(C)(C)c1ccc(-c2ccc(N(c3ccc4c(c3)-c3ccccc3C4(C)C)c3ccc4ccccc4c3-c3ccc4c(c3)oc3ccccc34)cc2)cc1 ; CC(C)(C)c1ccc(N(c2ccc3c(c2)-c2ccccc2C3(C)C)c2ccc3ccccc3c2-c2ccc3c(c2)oc2ccccc23)c2ccccc12 ; CC(C)(C)c1ccc(-c2ccc(-c3ccc(N(c4ccc5c(c4)-c4ccccc4C5(C)C)c4ccc5ccccc5c4-c4ccc5c(c4)oc4ccccc45)cc3)cc2)cc1]

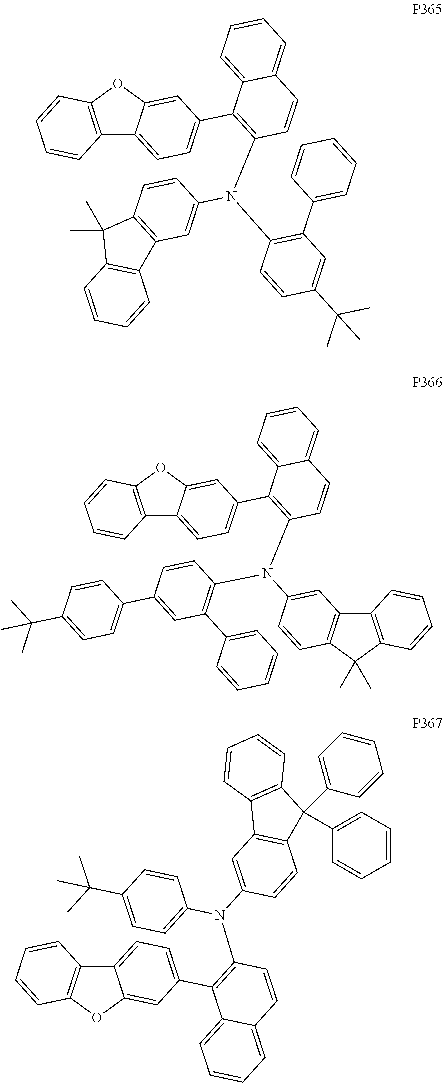

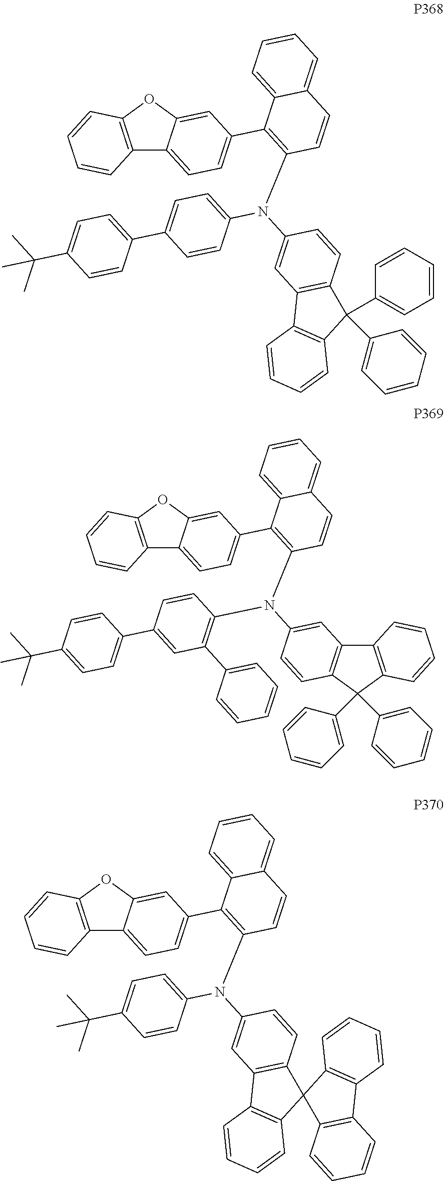 [SMILES: CC(C)(C)c1ccc(-c2ccc(N(c3ccc4c(c3)-c3ccccc3C4(c3ccccc3)c3ccccc3)c3ccc4ccccc4c3-c3ccc4c(c3)oc3ccccc34)cc2)cc1 ; CC(C)(C)c1ccc(-c2ccc(N(c3ccc4c(c3)-c3ccccc3C4(c3ccccc3)c3ccccc3)c3ccc4ccccc4c3-c3ccc4c(c3)oc3ccccc34)c(-c3ccccc3)c2)cc1 ; CC(C)(C)c1ccc(N(c2ccc3c(c2)-c2ccccc2C32c3ccccc3-c3ccccc32)c2ccc3ccccc3c2-c2ccc3c(c2)oc2ccccc23)cc1]

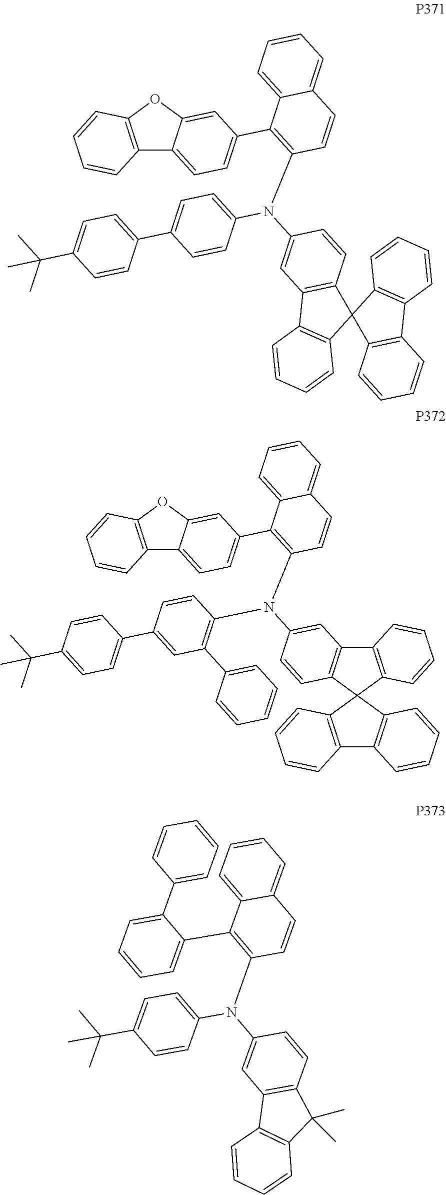 [SMILES: CC(C)(C)c1ccc(-c2ccc(N(c3ccc4c(c3)-c3ccccc3C43c4ccccc4-c4ccccc43)c3ccc4ccccc4c3-c3ccc4c(c3)oc3ccccc34)cc2)cc1 ; CC(C)(C)c1ccc(-c2ccc(N(c3ccc4c(c3)-c3ccccc3C43c4ccccc4-c4ccccc43)c3ccc4ccccc4c3-c3ccc4c(c3)oc3ccccc34)c(-c3ccccc3)c2)cc1 ; CC(C)(C)c1ccc(N(c2ccc3c(c2)-c2ccccc2C3(C)C)c2ccc3ccccc3c2-c2ccccc2-c2ccccc2)cc1]

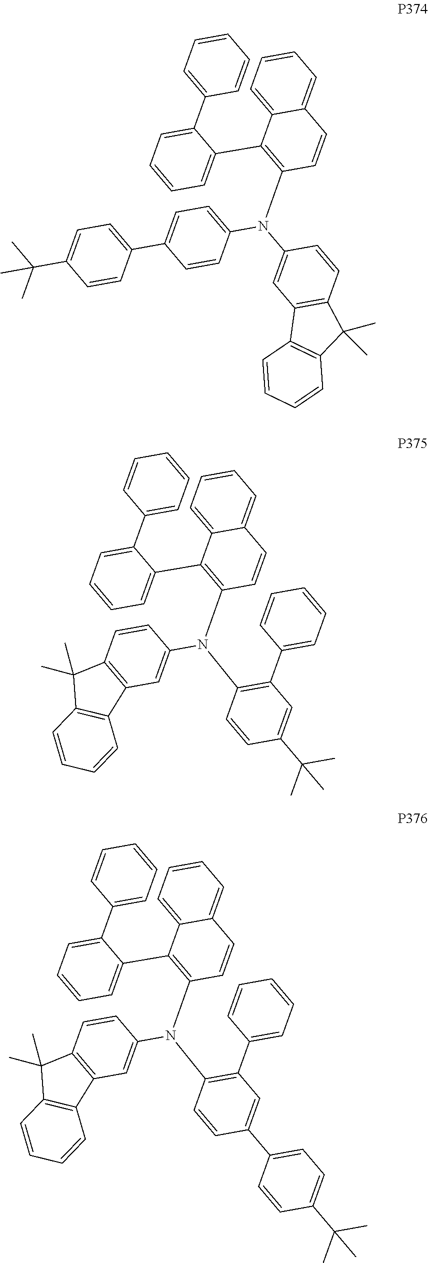

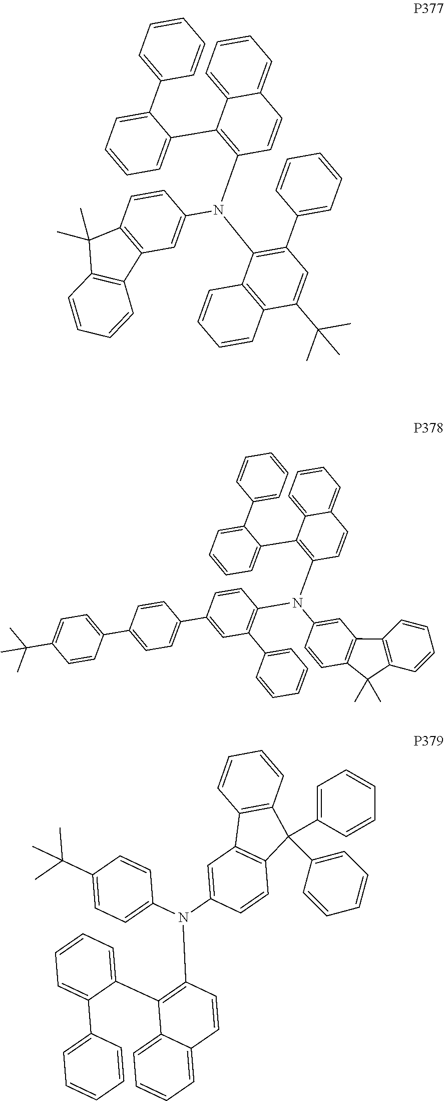

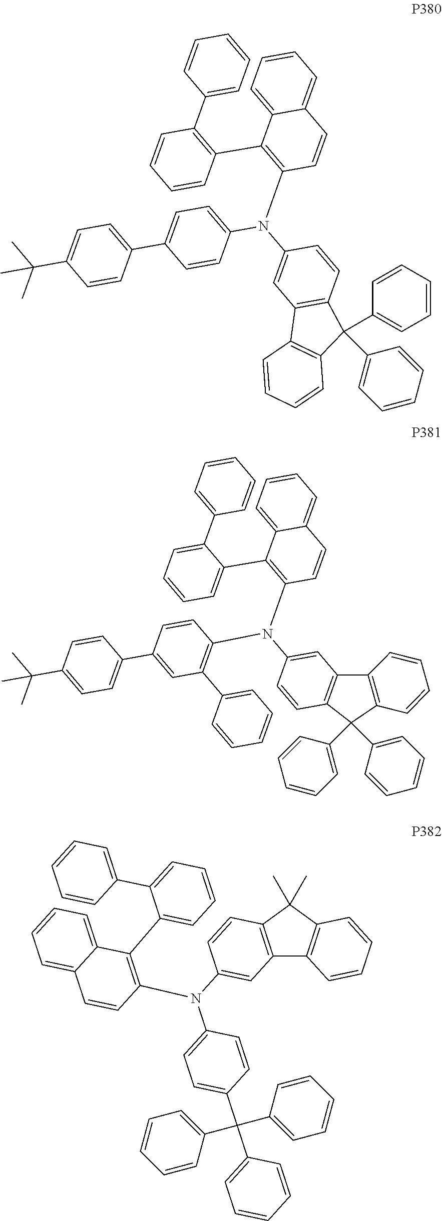 [SMILES: CC(C)(C)c1ccc(-c2ccc(N(c3ccc4c(c3)-c3ccccc3C4(c3ccccc3)c3ccccc3)c3ccc4ccccc4c3-c3ccccc3-c3ccccc3)cc2)cc1 ; CC(C)(C)c1ccc(-c2ccc(N(c3ccc4c(c3)-c3ccccc3C4(c3ccccc3)c3ccccc3)c3ccc4ccccc4c3-c3ccccc3-c3ccccc3)c(-c3ccccc3)c2)cc1 ; CC1(C)c2ccccc2-c2cc(N(c3ccc(C(c4ccccc4)(c4ccccc4)c4ccccc4)cc3)c3ccc4ccccc4c3-c3ccccc3-c3ccccc3)ccc21]

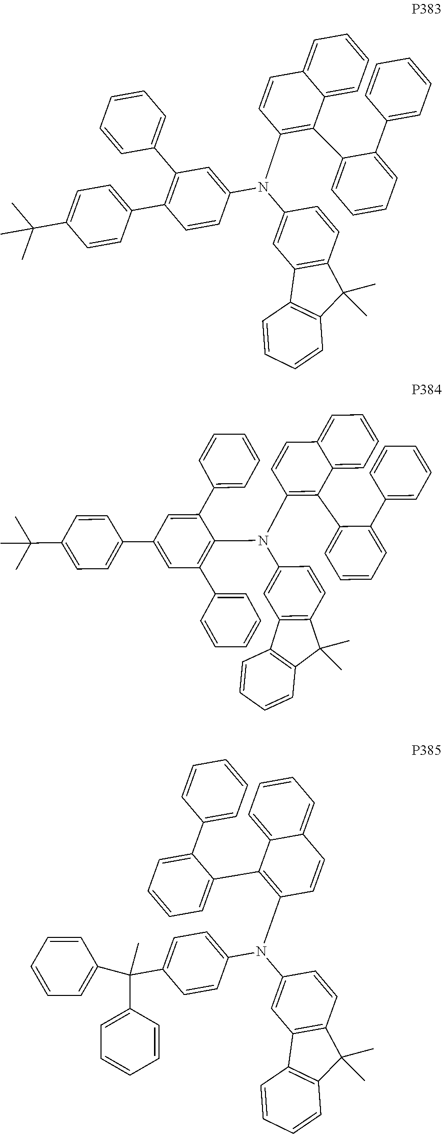 [SMILES: CC(C)(C)c1ccc(-c2ccc(N(c3ccc4c(c3)-c3ccccc3C4(C)C)c3ccc4ccccc4c3-c3ccccc3-c3ccccc3)cc2-c2ccccc2)cc1 ; CC(C)(C)c1ccc(-c2cc(-c3ccccc3)c(N(c3ccc4c(c3)-c3ccccc3C4(C)C)c3ccc4ccccc4c3-c3ccccc3-c3ccccc3)c(-c3ccccc3)c2)cc1 ; CC1(C)c2ccccc2-c2cc(N(c3ccc(C(C)(c4ccccc4)c4ccccc4)cc3)c3ccc4ccccc4c3-c3ccccc3-c3ccccc3)ccc21]

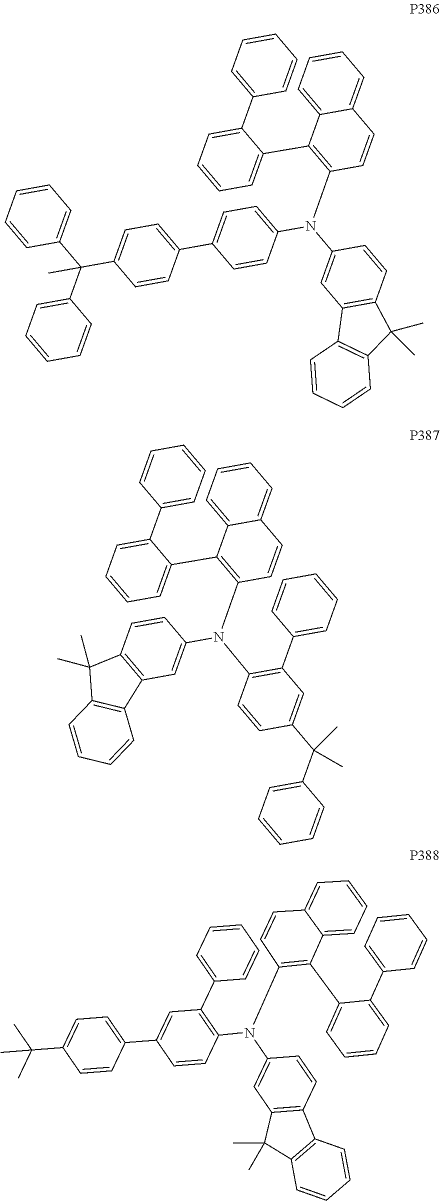

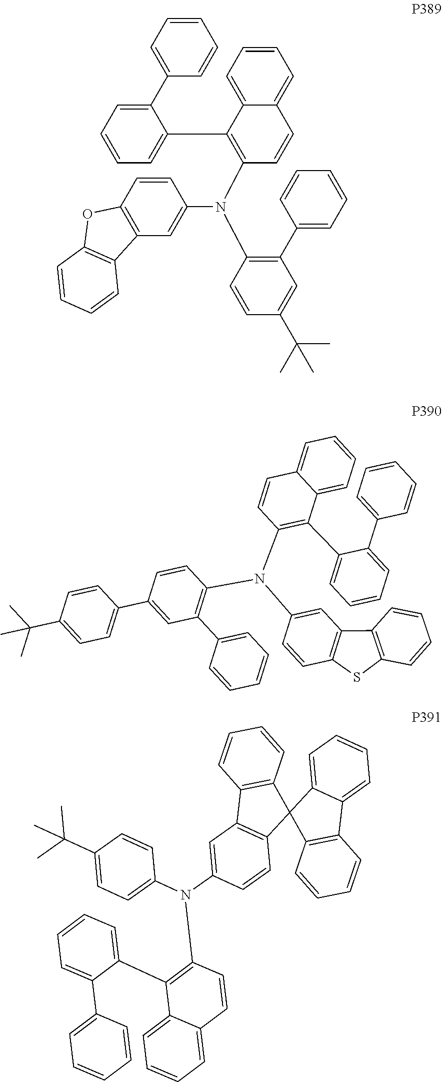

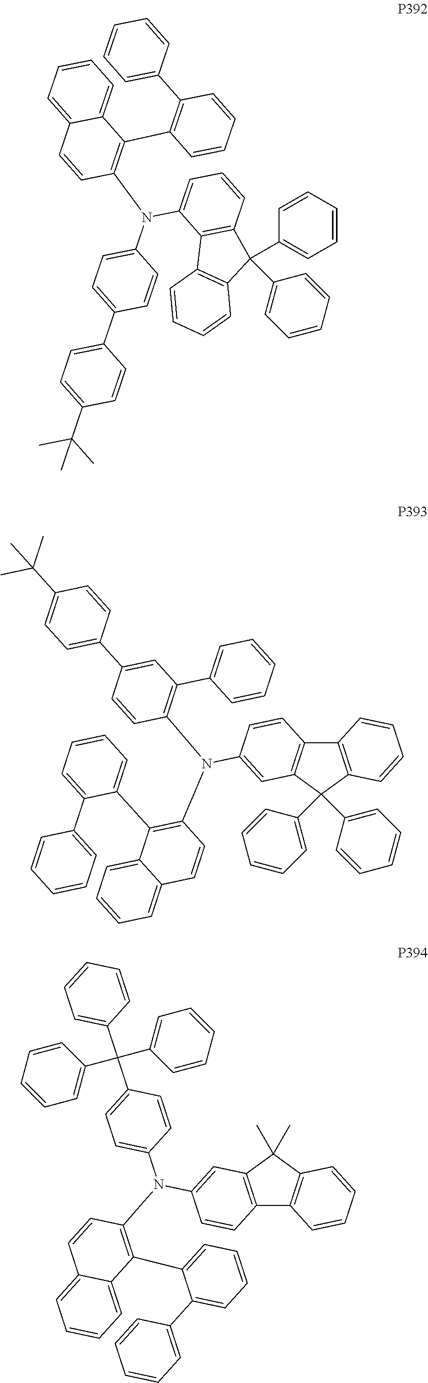

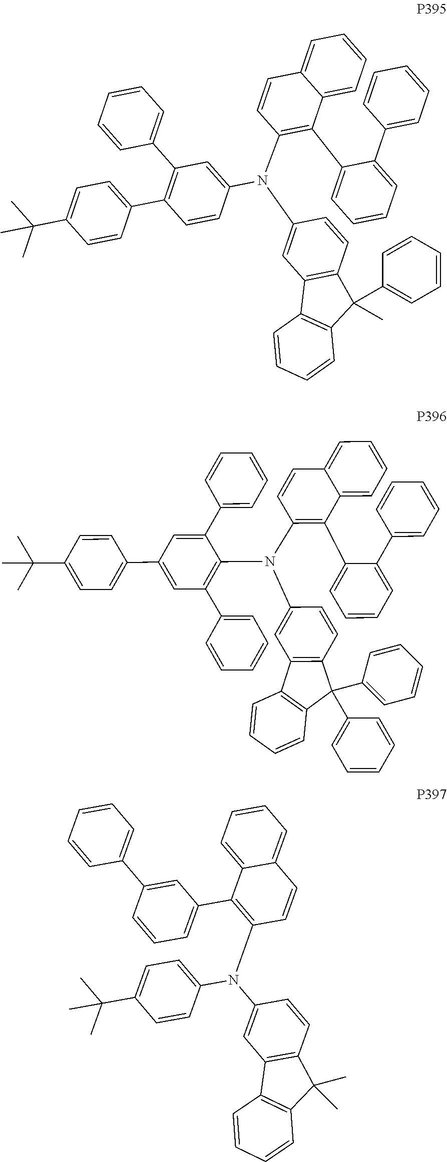 [SMILES: CC(C)(C)c1ccc(-c2ccc(N(c3ccc4c(c3)-c3ccccc3C4(C)c3ccccc3)c3ccc4ccccc4c3-c3ccccc3-c3ccccc3)cc2-c2ccccc2)cc1 ; CC(C)(C)c1ccc(-c2cc(-c3ccccc3)c(N(c3ccc4c(c3)-c3ccccc3C4(c3ccccc3)c3ccccc3)c3ccc4ccccc4c3-c3ccccc3-c3ccccc3)c(-c3ccccc3)c2)cc1 ; CC(C)(C)c1ccc(N(c2ccc3c(c2)-c2ccccc2C3(C)C)c2ccc3ccccc3c2-c2cccc(-c3ccccc3)c2)cc1]

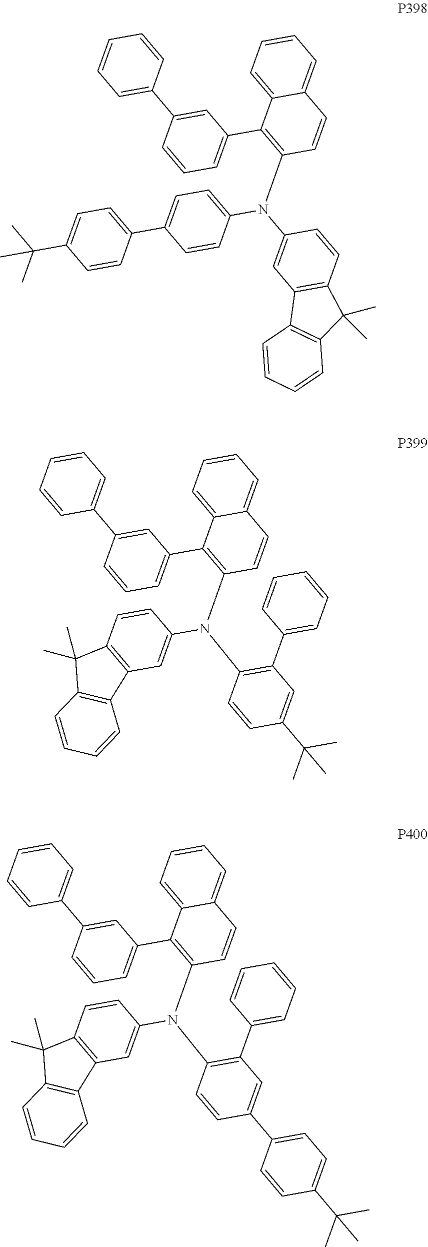 [SMILES: CC(C)(C)c1ccc(-c2ccc(N(c3ccc4c(c3)-c3ccccc3C4(C)C)c3ccc4ccccc4c3-c3cccc(-c4ccccc4)c3)cc2)cc1 ; CC(C)(C)c1ccc(N(c2ccc3c(c2)-c2ccccc2C3(C)C)c2ccc3ccccc3c2-c2cccc(-c3ccccc3)c2)c(-c2ccccc2)c1 ; CC(C)(C)c1ccc(-c2ccc(N(c3ccc4c(c3)-c3ccccc3C4(C)C)c3ccc4ccccc4c3-c3cccc(-c4ccccc4)c3)c(-c3ccccc3)c2)cc1]

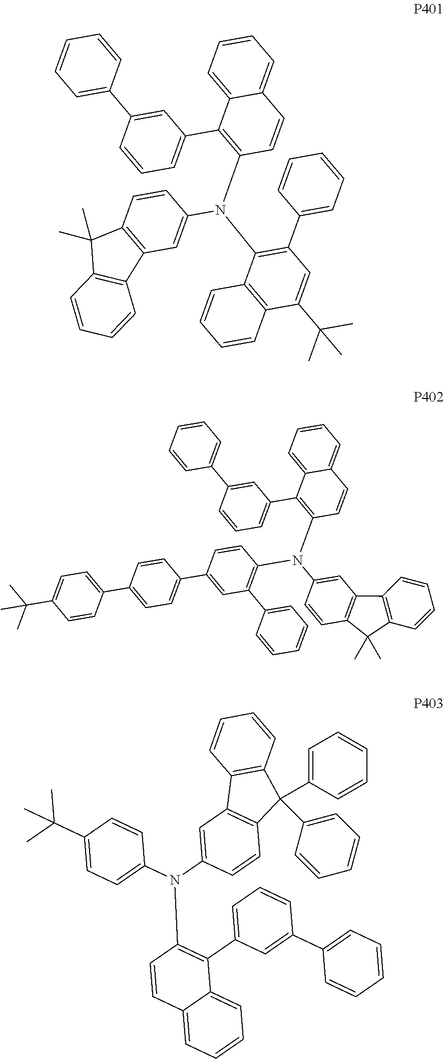

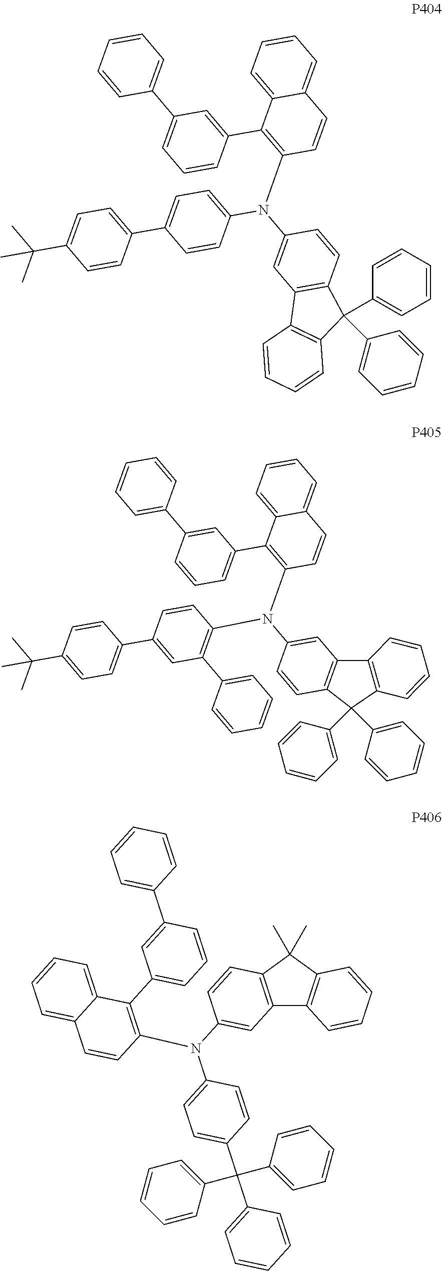 [SMILES: CC(C)(C)c1ccc(-c2ccc(N(c3ccc4c(c3)-c3ccccc3C4(c3ccccc3)c3ccccc3)c3ccc4ccccc4c3-c3cccc(-c4ccccc4)c3)cc2)cc1 ; CC(C)(C)c1ccc(-c2ccc(N(c3ccc4c(c3)-c3ccccc3C4(c3ccccc3)c3ccccc3)c3ccc4ccccc4c3-c3cccc(-c4ccccc4)c3)c(-c3ccccc3)c2)cc1 ; CC1(C)c2ccccc2-c2cc(N(c3ccc(C(c4ccccc4)(c4ccccc4)c4ccccc4)cc3)c3ccc4ccccc4c3-c3cccc(-c4ccccc4)c3)ccc21]

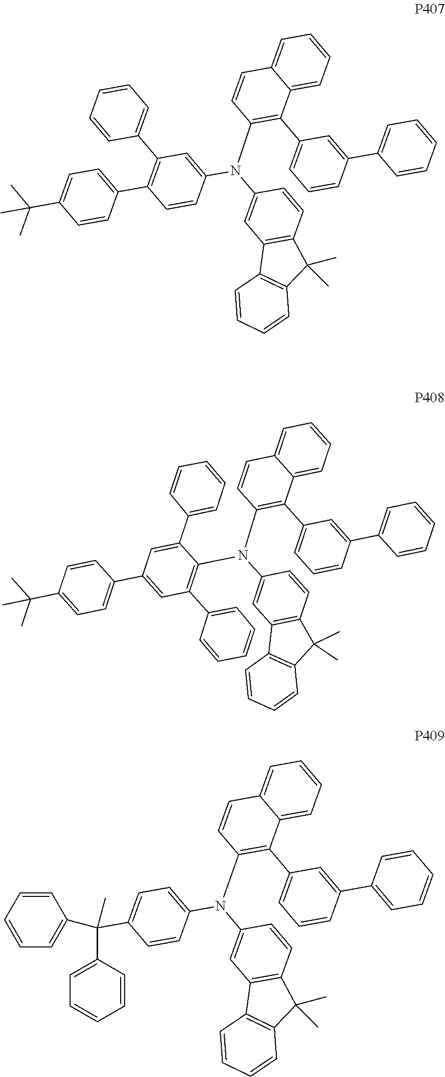 [SMILES: CC(C)(C)c1ccc(-c2ccc(N(c3ccc4c(c3)-c3ccccc3C4(C)C)c3ccc4ccccc4c3-c3cccc(-c4ccccc4)c3)cc2-c2ccccc2)cc1 ; CC(C)(C)c1ccc(-c2cc(-c3ccccc3)c(N(c3ccc4c(c3)-c3ccccc3C4(C)C)c3ccc4ccccc4c3-c3cccc(-c4ccccc4)c3)c(-c3ccccc3)c2)cc1 ; CC1(C)c2ccccc2-c2cc(N(c3ccc(C(C)(c4ccccc4)c4ccccc4)cc3)c3ccc4ccccc4c3-c3cccc(-c4ccccc4)c3)ccc21]

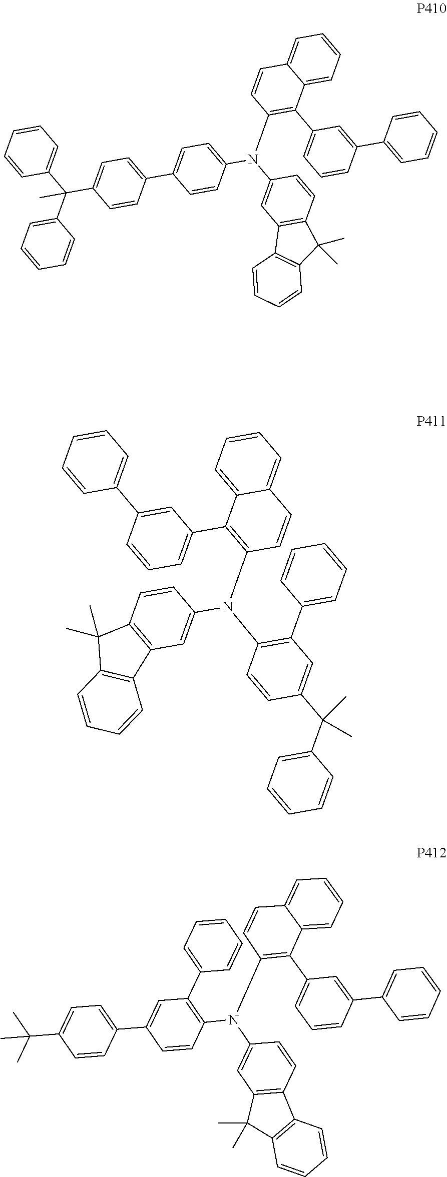

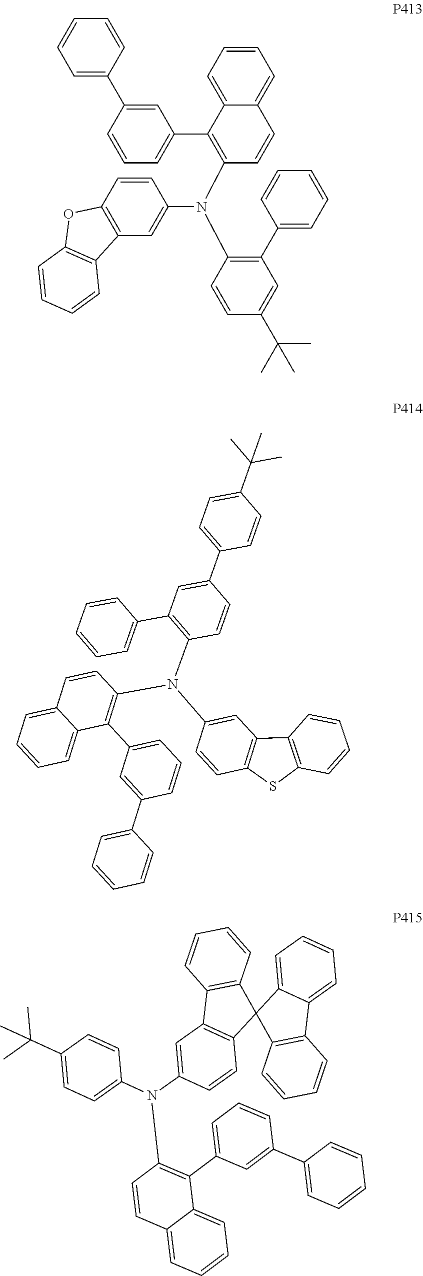 [SMILES: CC(C)(C)c1ccc(N(c2ccc3oc4ccccc4c3c2)c2ccc3ccccc3c2-c2cccc(-c3ccccc3)c2)c(-c2ccccc2)c1 ; CC(C)(C)c1ccc(-c2ccc(N(c3ccc4sc5ccccc5c4c3)c3ccc4ccccc4c3-c3cccc(-c4ccccc4)c3)c(-c3ccccc3)c2)cc1 ; CC(C)(C)c1ccc(N(c2ccc3c(c2)-c2ccccc2C32c3ccccc3-c3ccccc32)c2ccc3ccccc3c2-c2cccc(-c3ccccc3)c2)cc1]

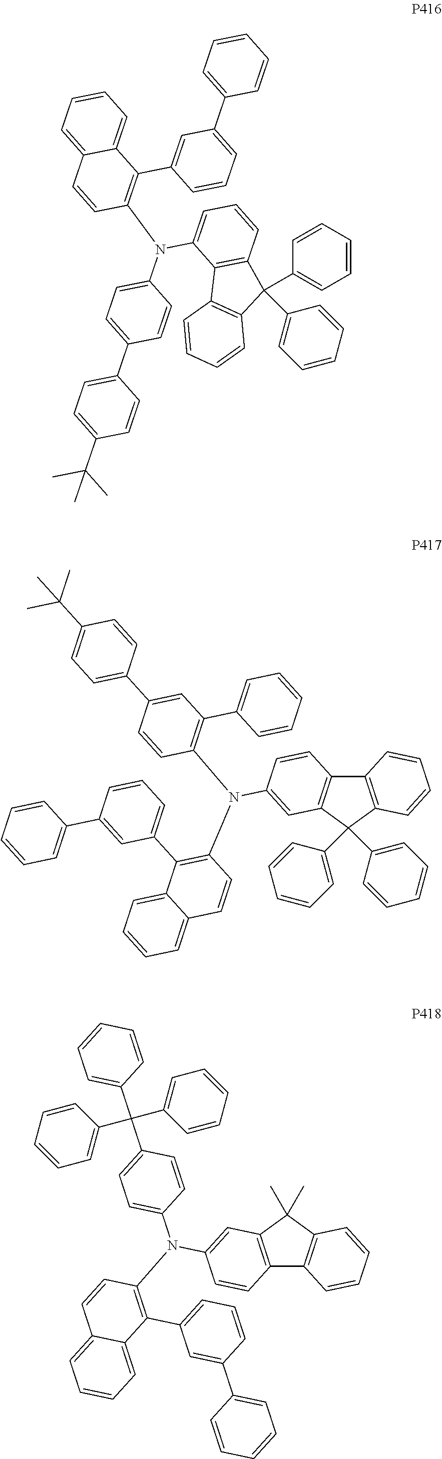 [SMILES: CC(C)(C)c1ccc(-c2ccc(N(c3cccc4c3-c3ccccc3C4(c3ccccc3)c3ccccc3)c3ccc4ccccc4c3-c3cccc(-c4ccccc4)c3)cc2)cc1 ; CC(C)(C)c1ccc(-c2ccc(N(c3ccc4c(c3)C(c3ccccc3)(c3ccccc3)c3ccccc3-4)c3ccc4ccccc4c3-c3cccc(-c4ccccc4)c3)c(-c3ccccc3)c2)cc1 ; CC1(C)c2ccccc2-c2ccc(N(c3ccc(C(c4ccccc4)(c4ccccc4)c4ccccc4)cc3)c3ccc4ccccc4c3-c3cccc(-c4ccccc4)c3)cc21]

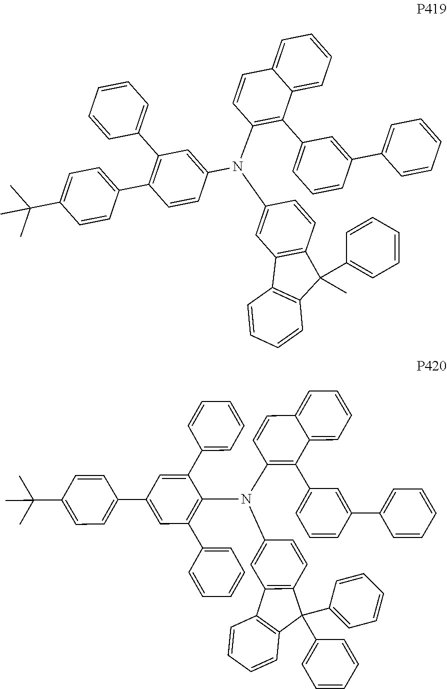 [SMILES: CC(C)(C)c1ccc(-c2ccc(N(c3ccc4c(c3)-c3ccccc3C4(C)c3ccccc3)c3ccc4ccccc4c3-c3cccc(-c4ccccc4)c3)cc2-c2ccccc2)cc1 ; CC(C)(C)c1ccc(-c2cc(-c3ccccc3)c(N(c3ccc4c(c3)-c3ccccc3C4(c3ccccc3)c3ccccc3)c3ccc4ccccc4c3-c3cccc(-c4ccccc4)c3)c(-c3ccccc3)c2)cc1]

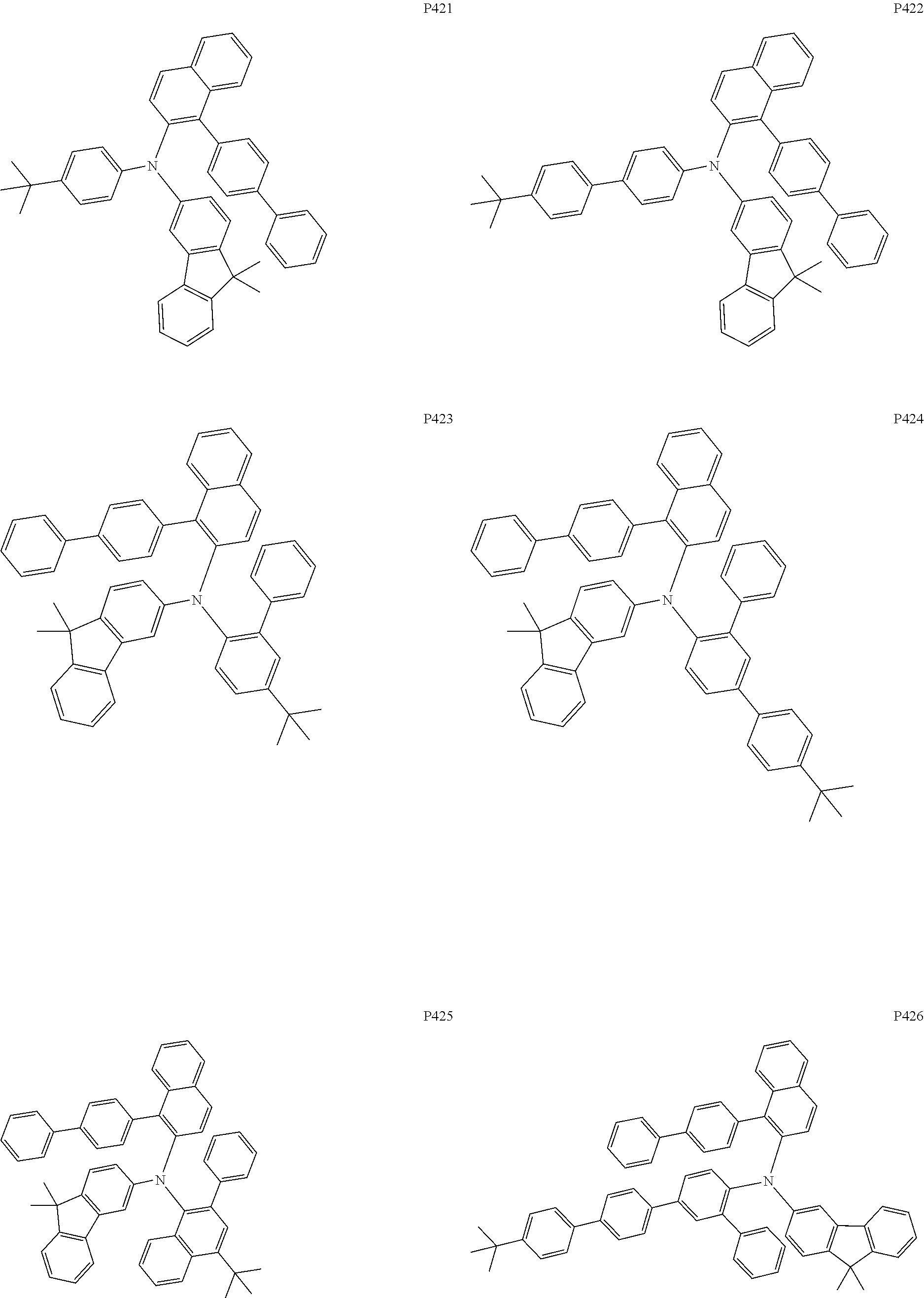

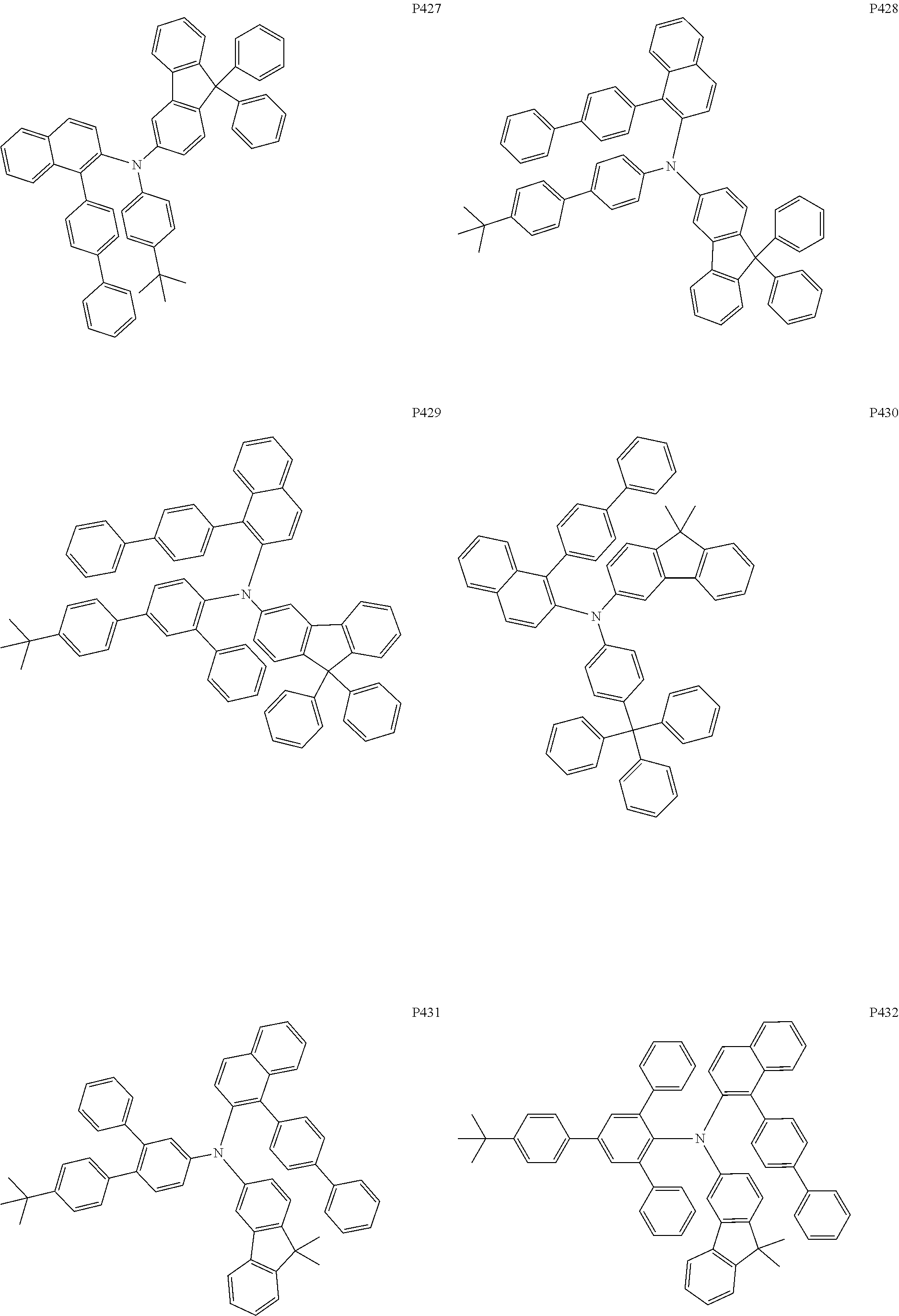

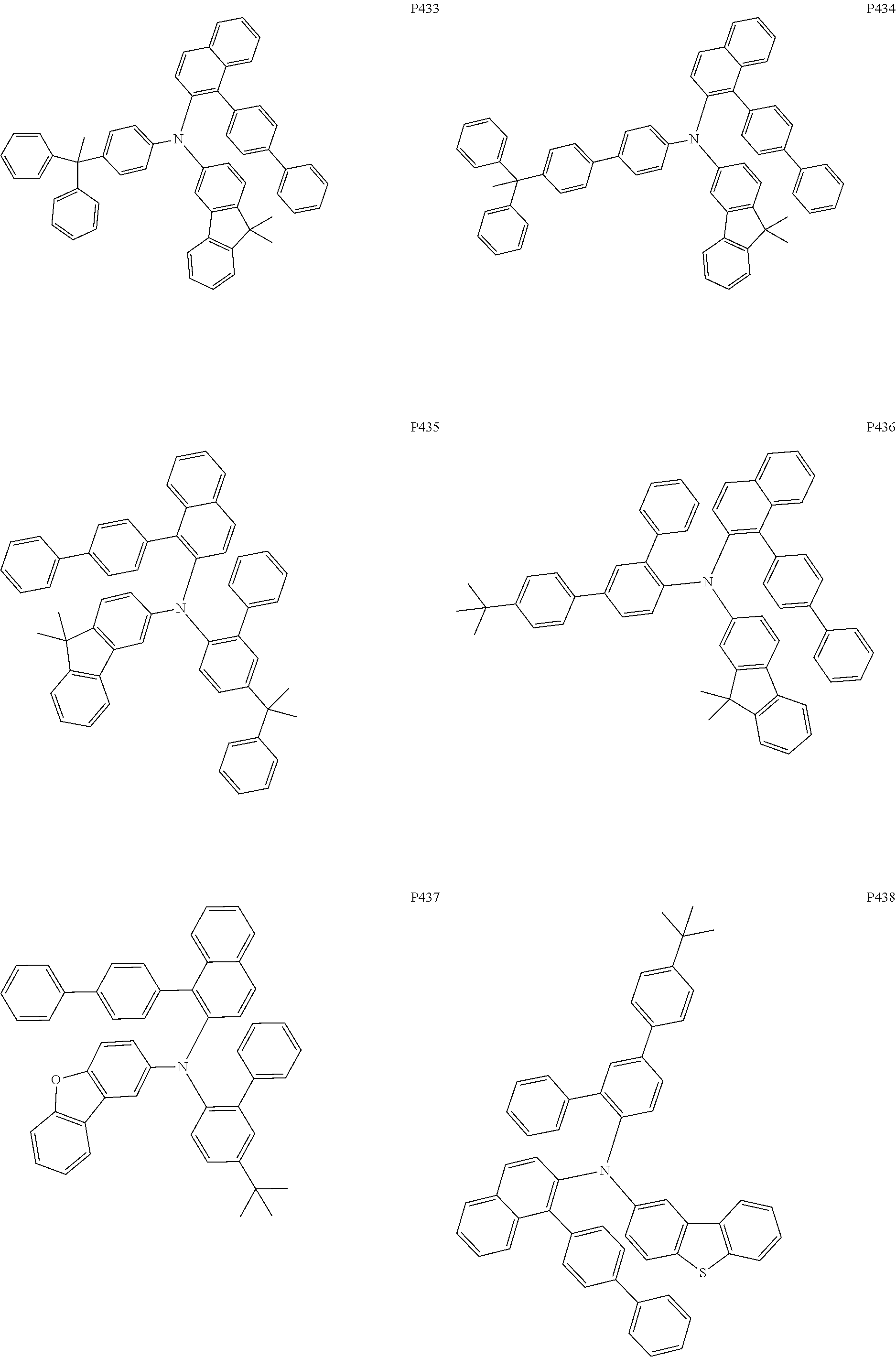

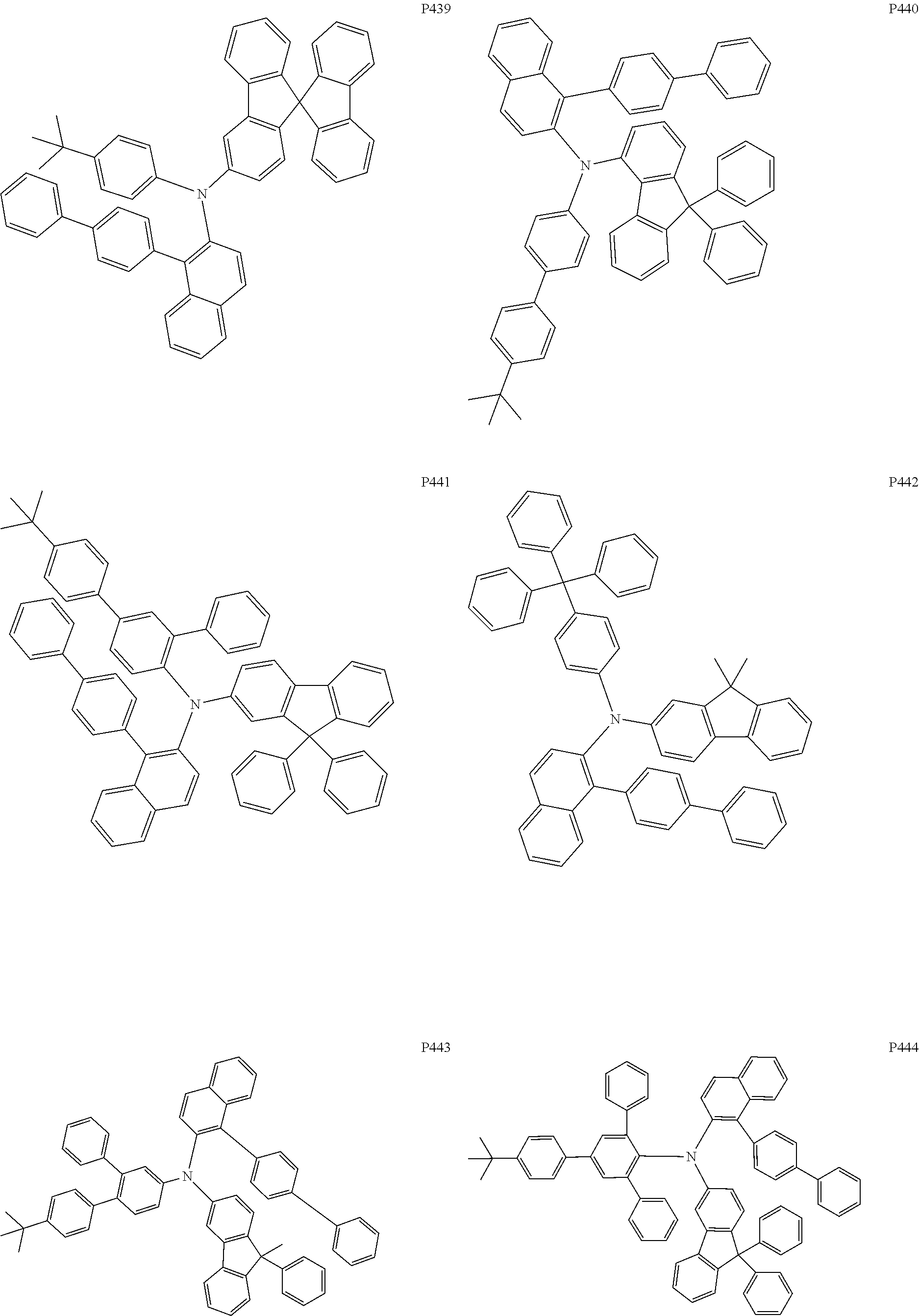

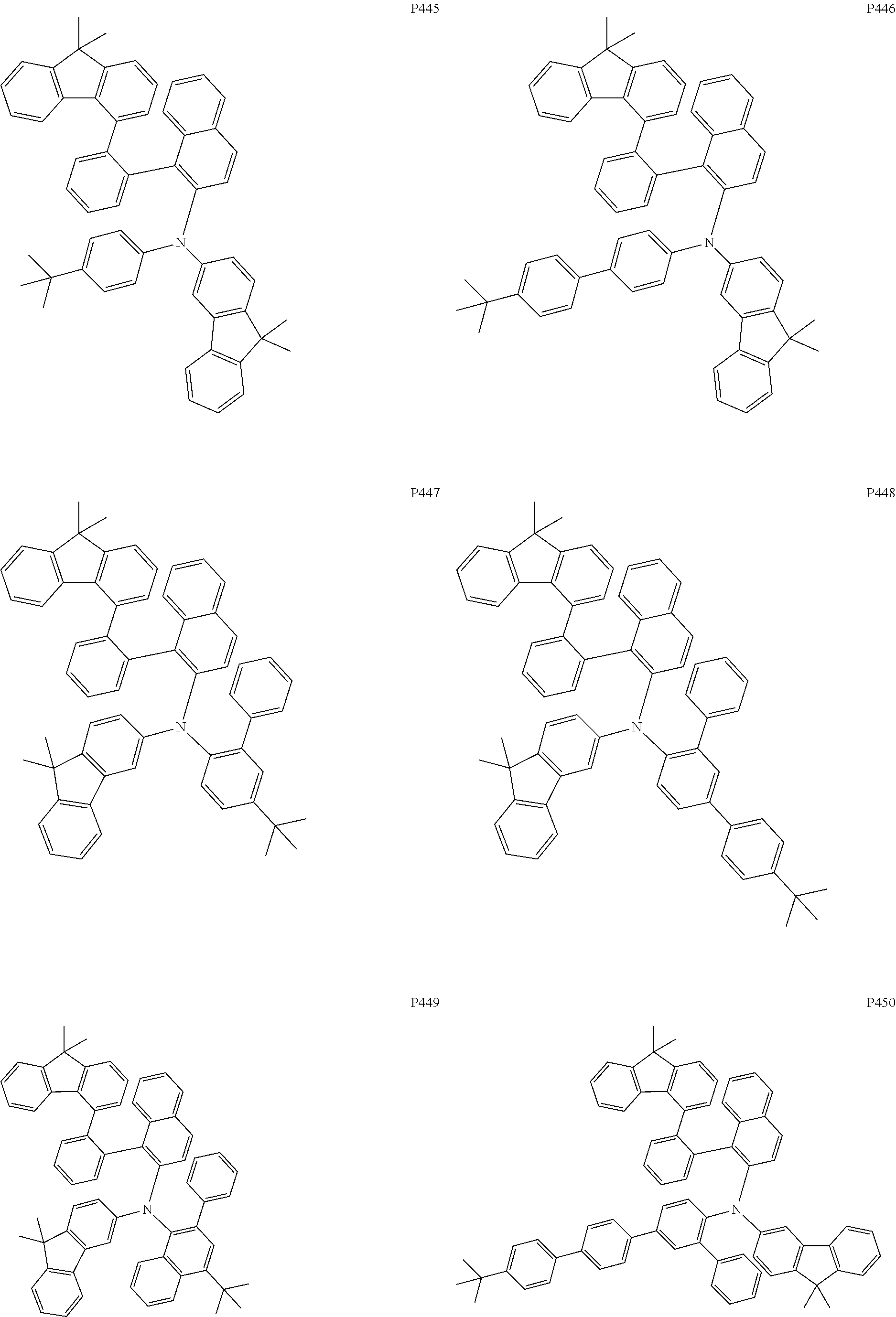

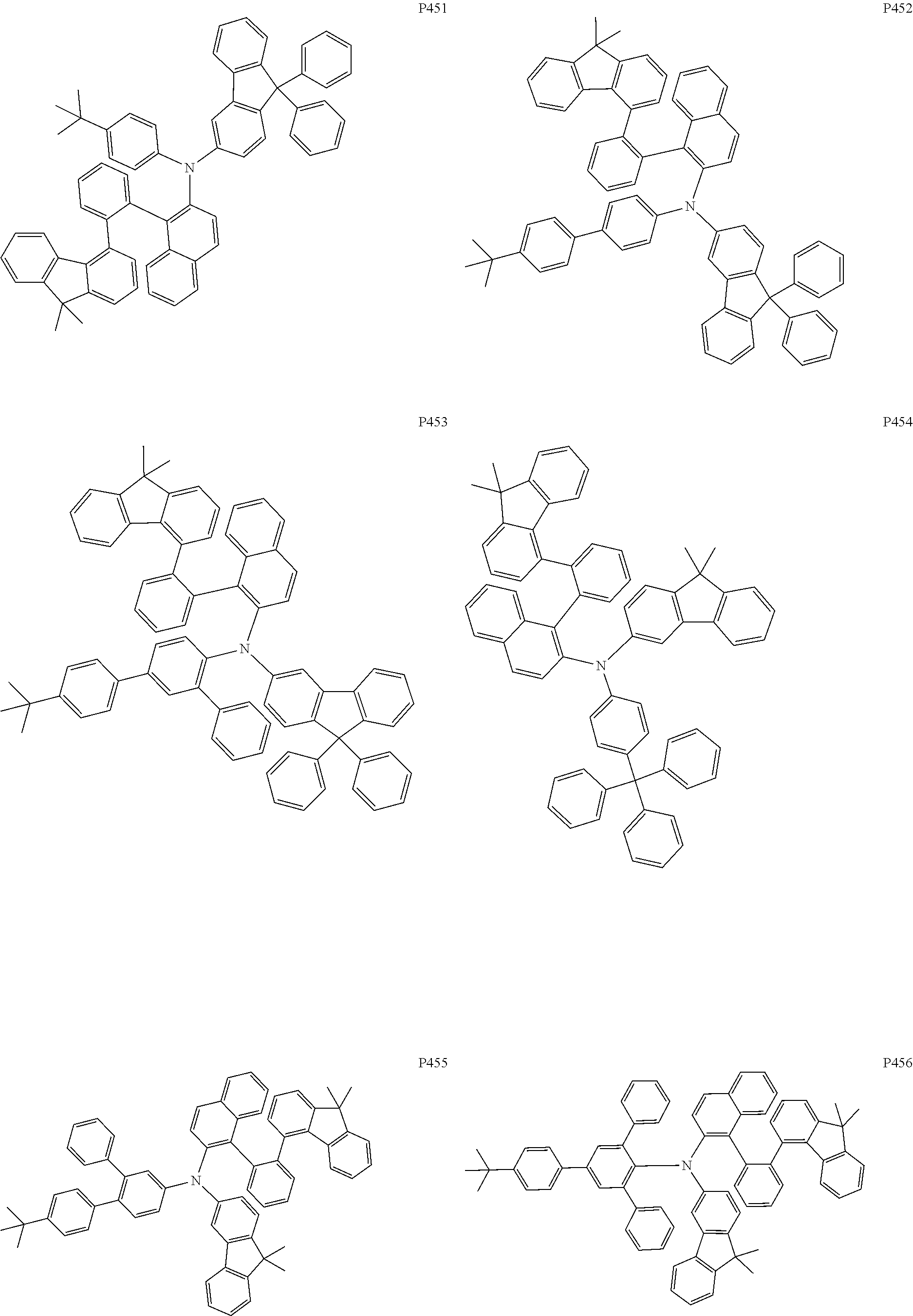

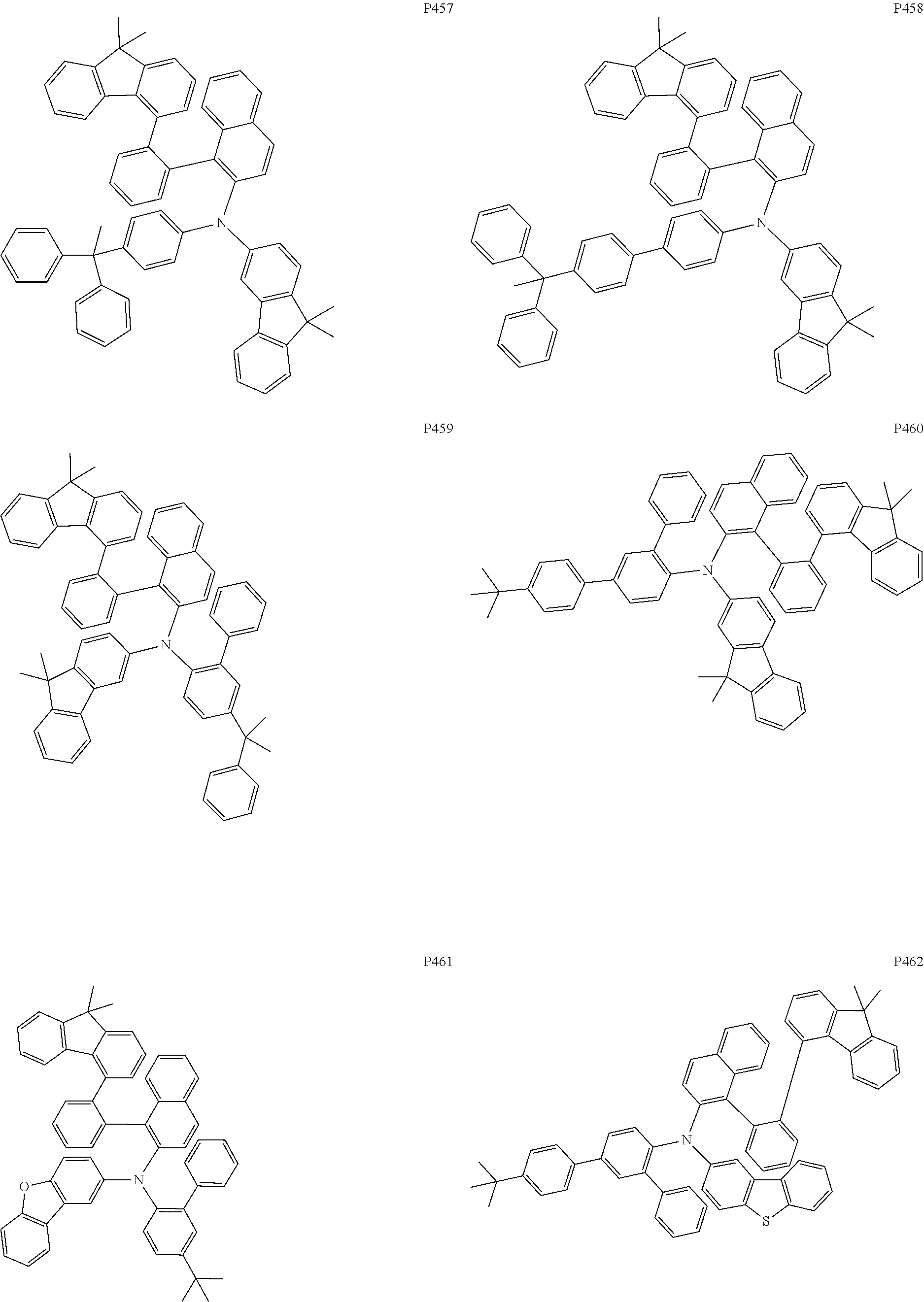

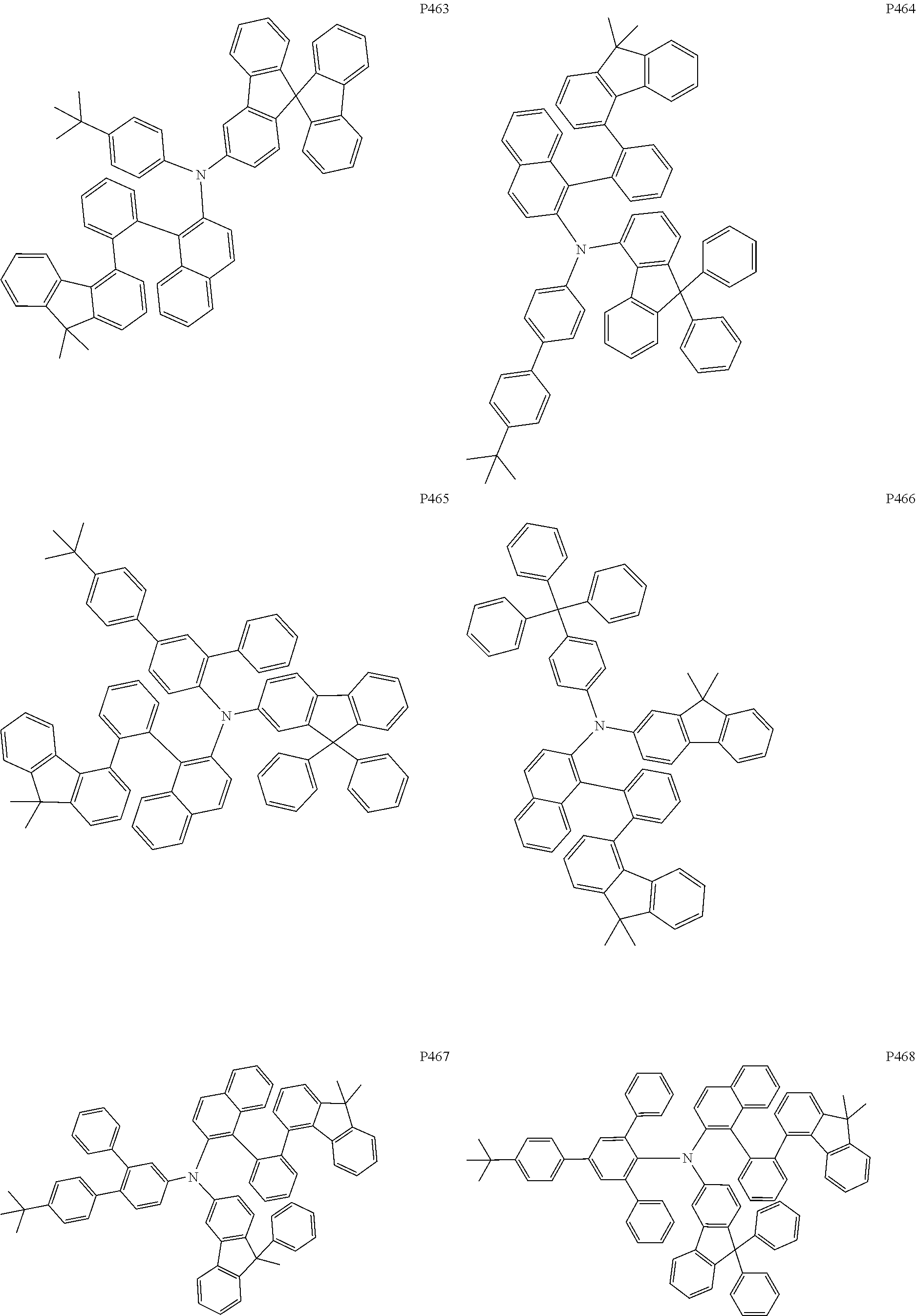

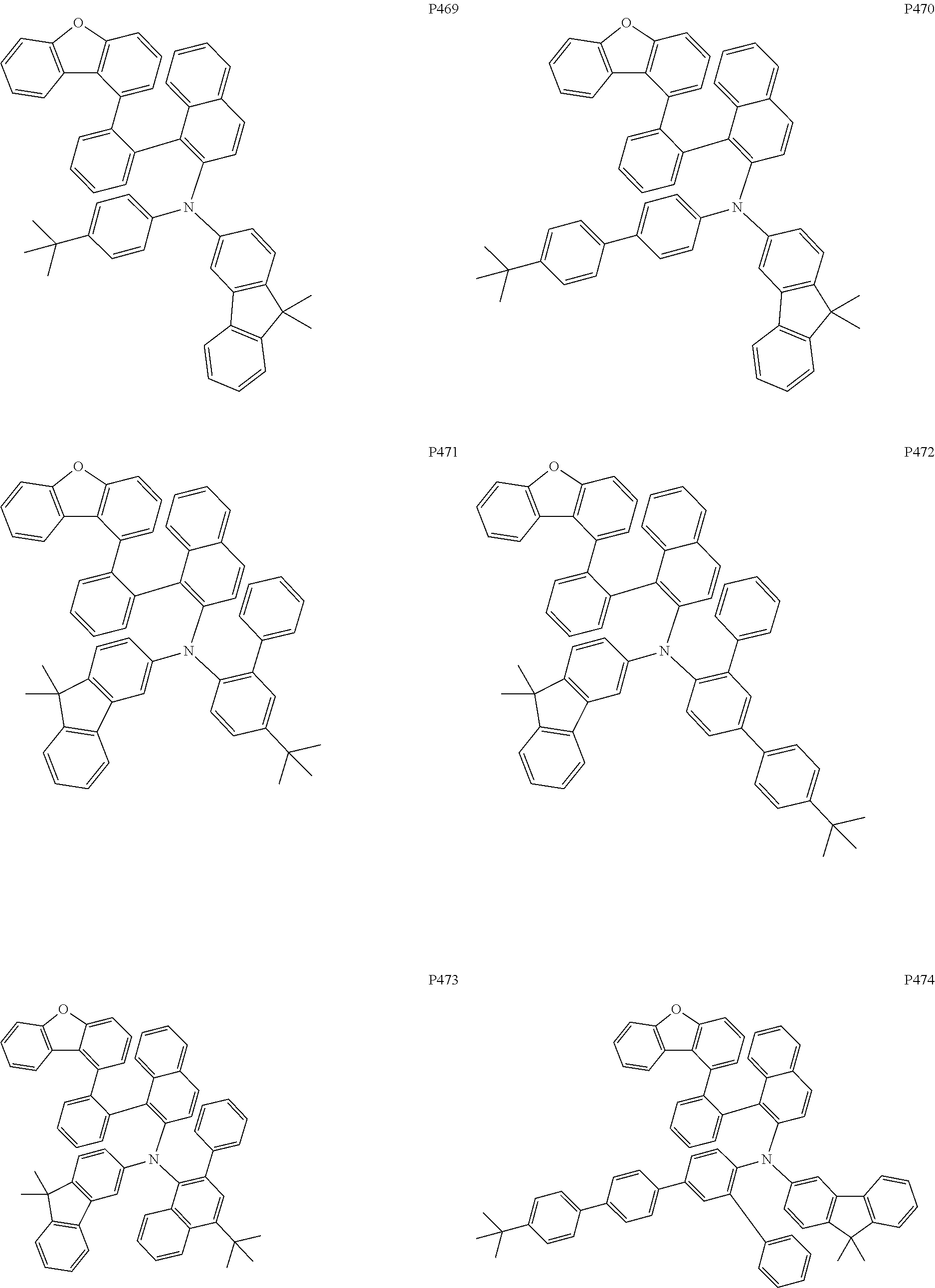

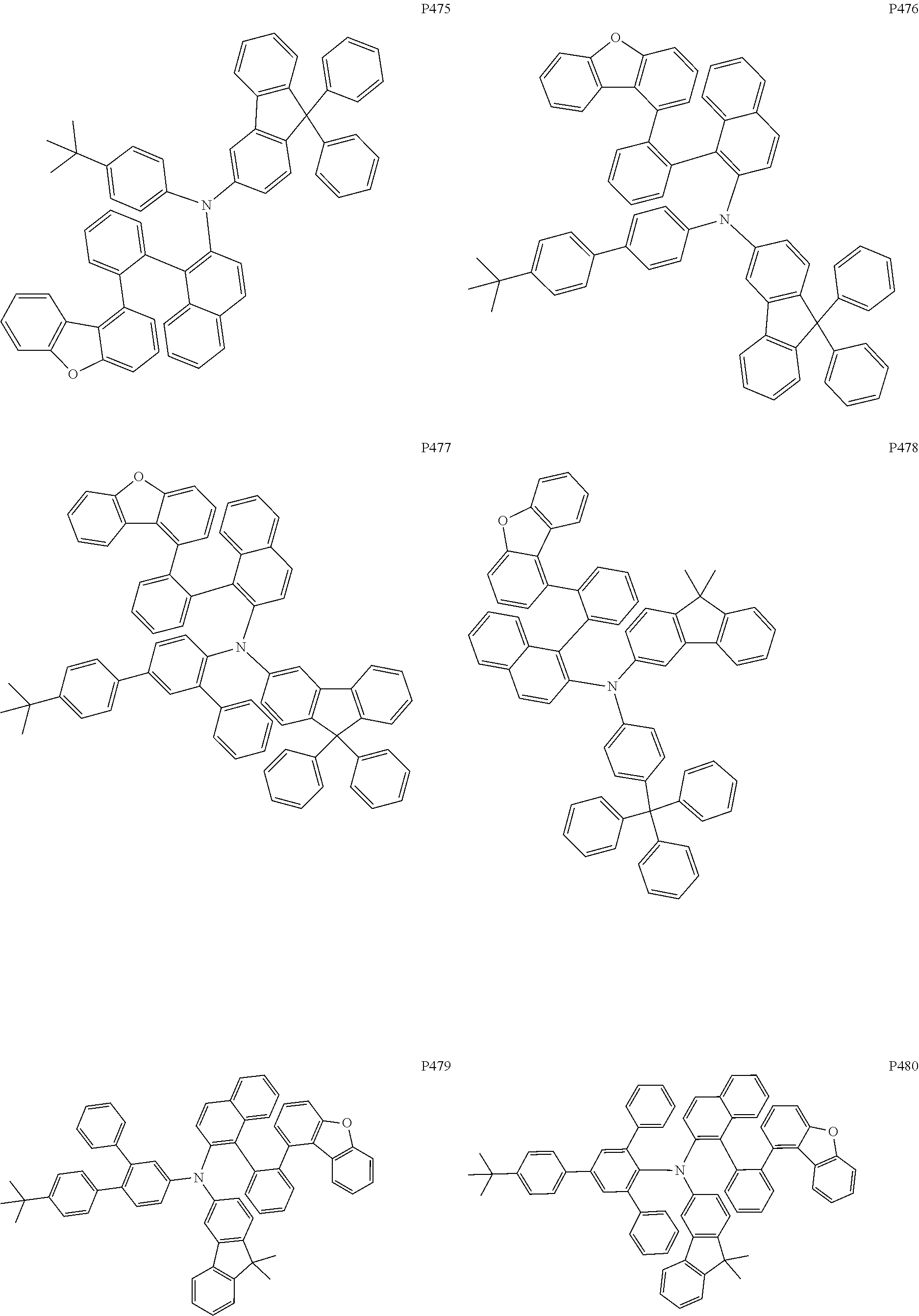

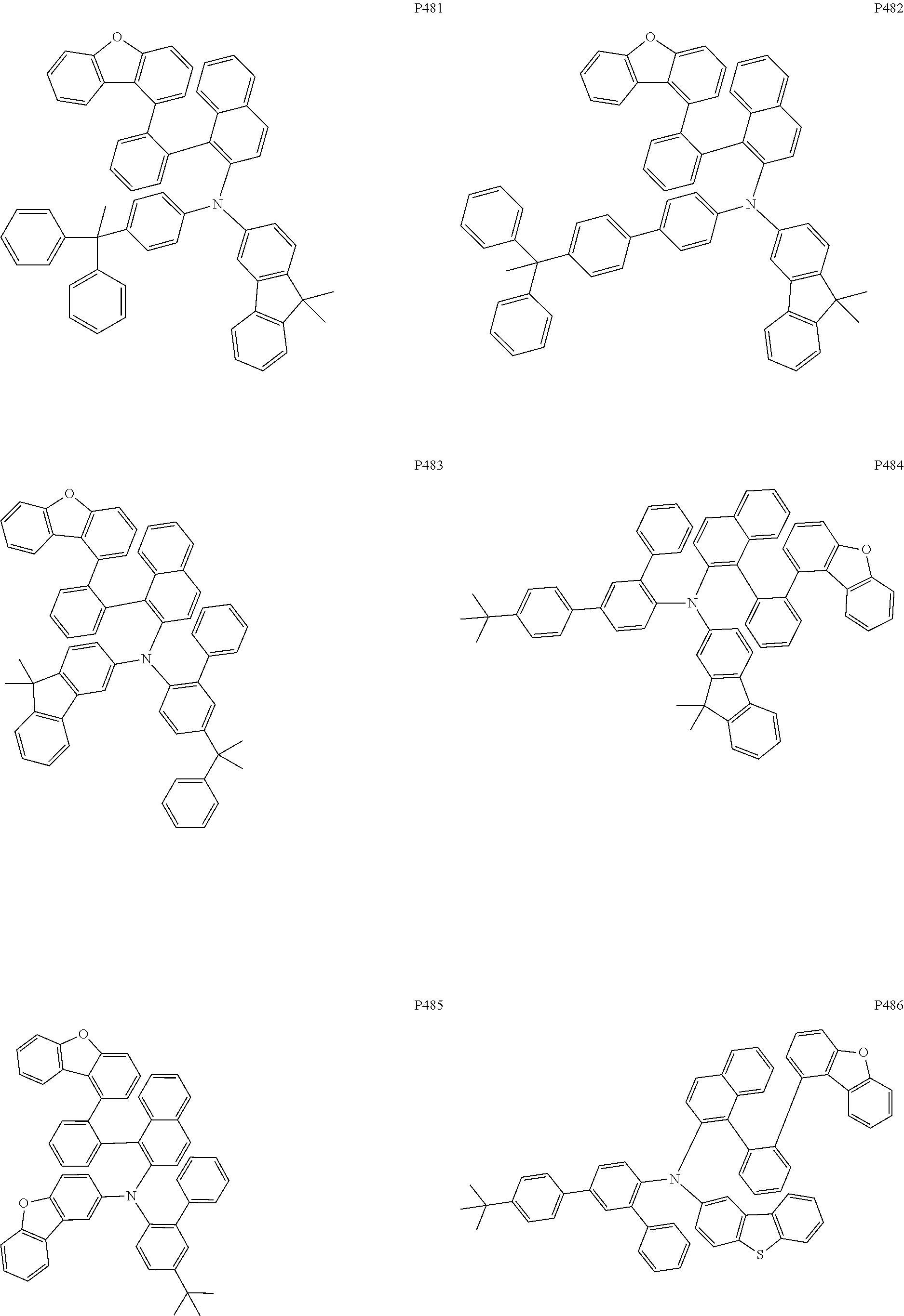

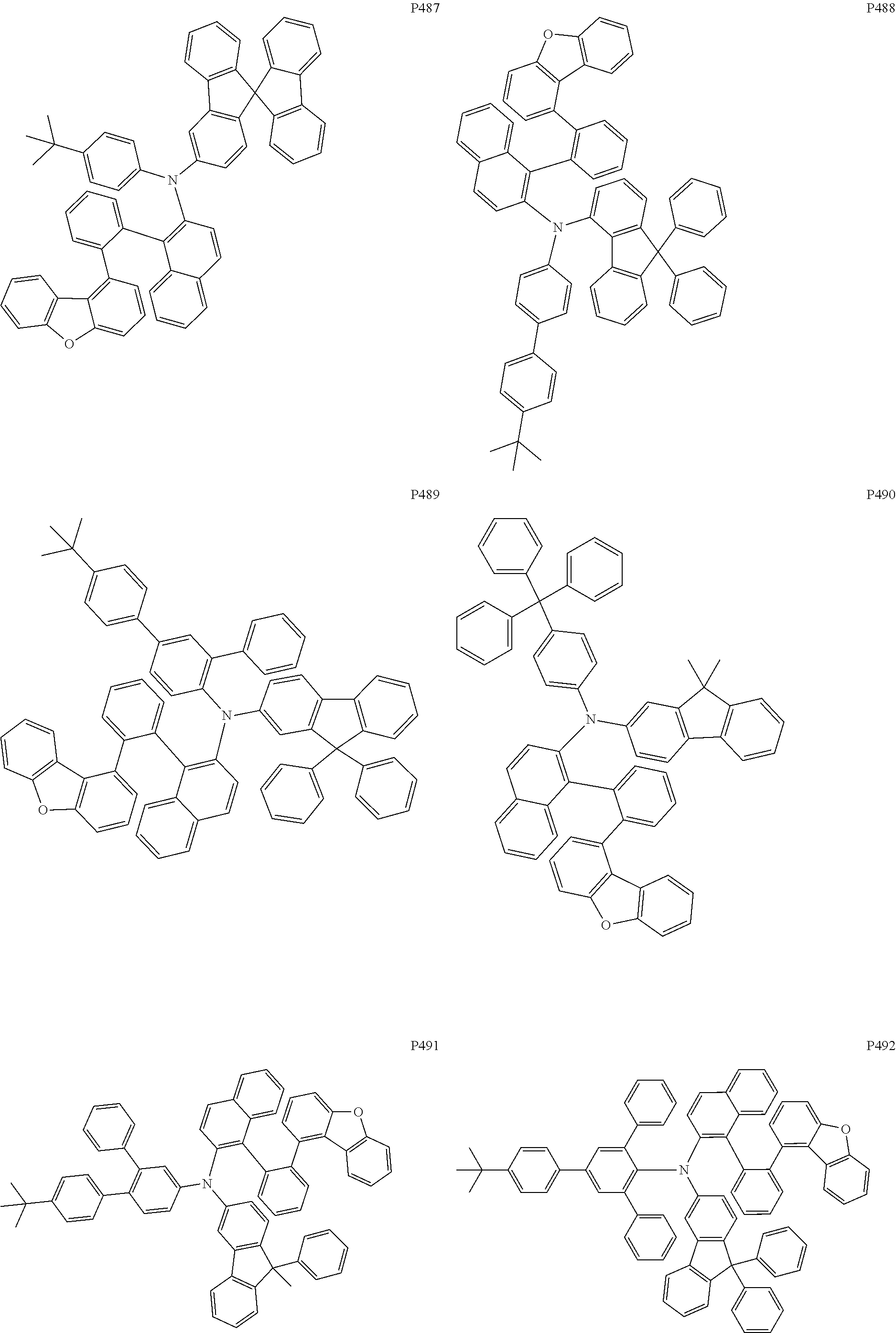

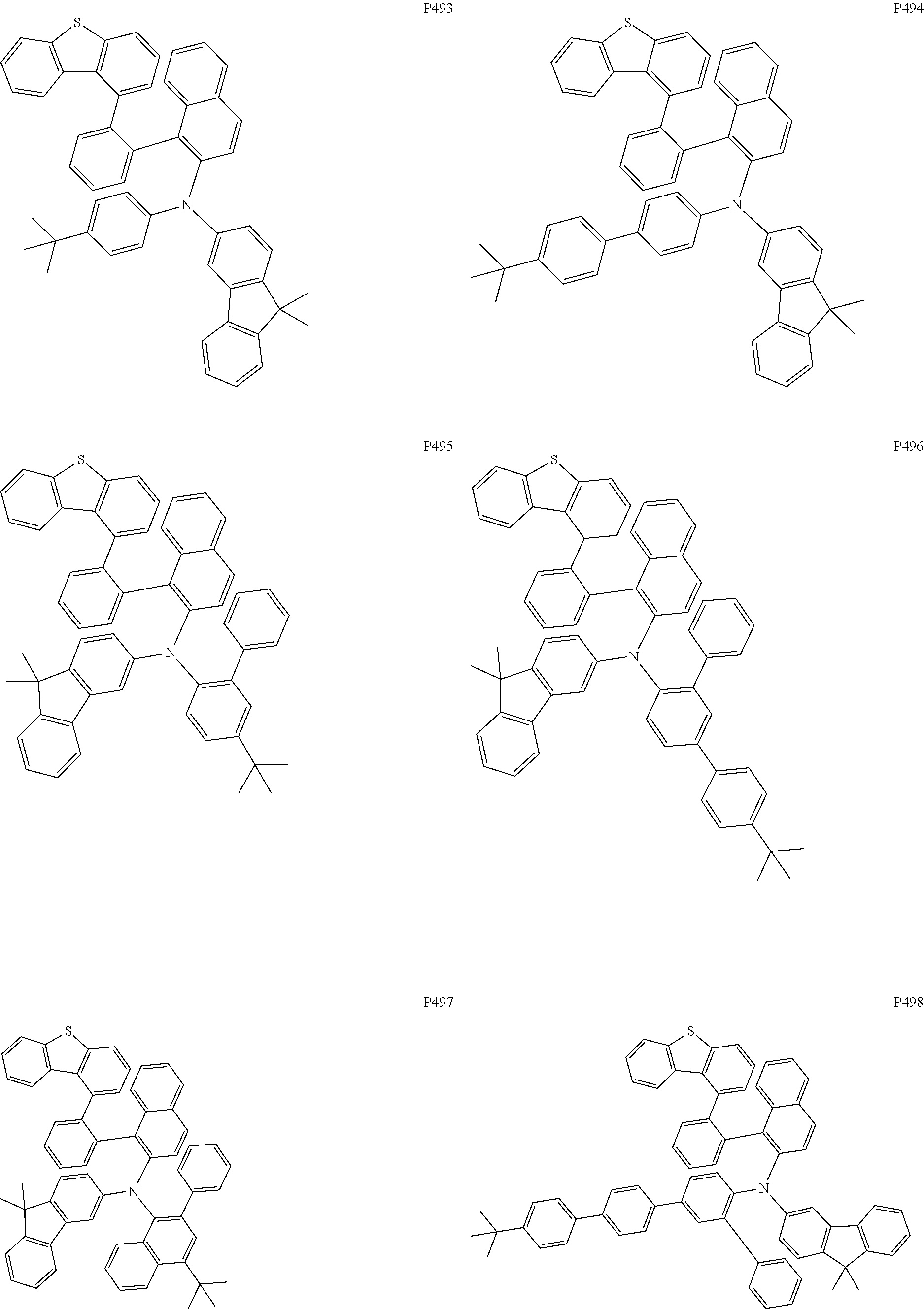

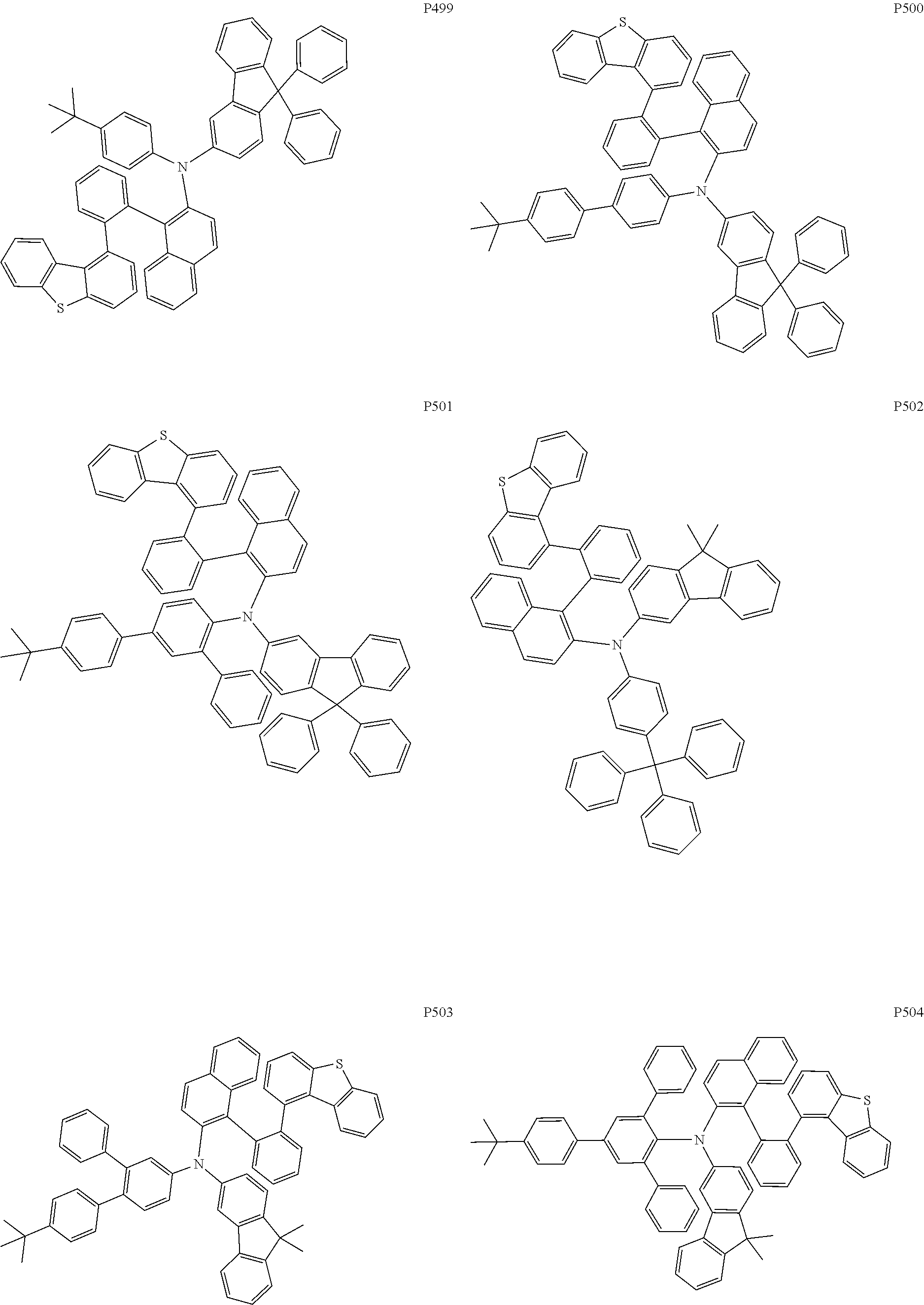

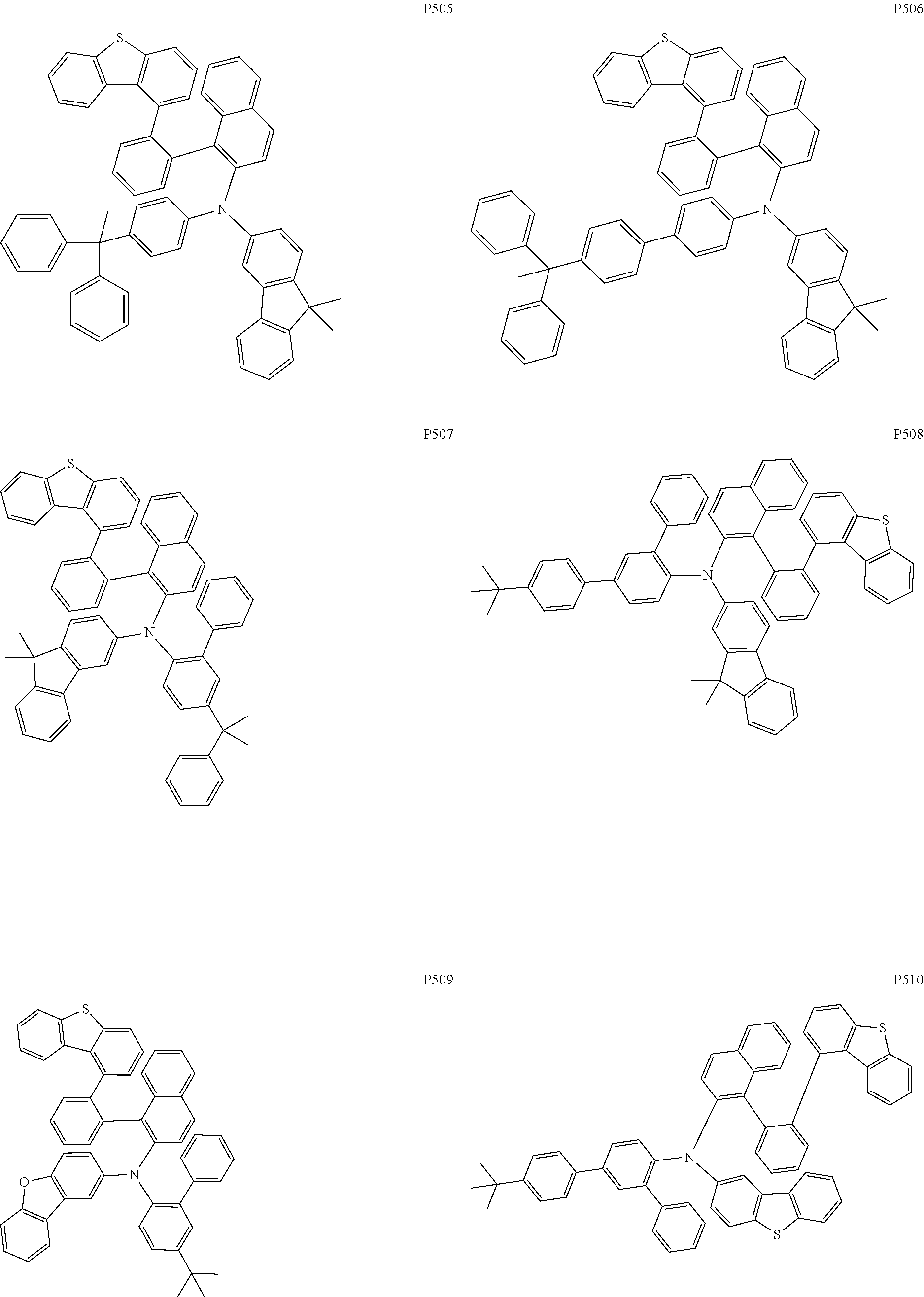

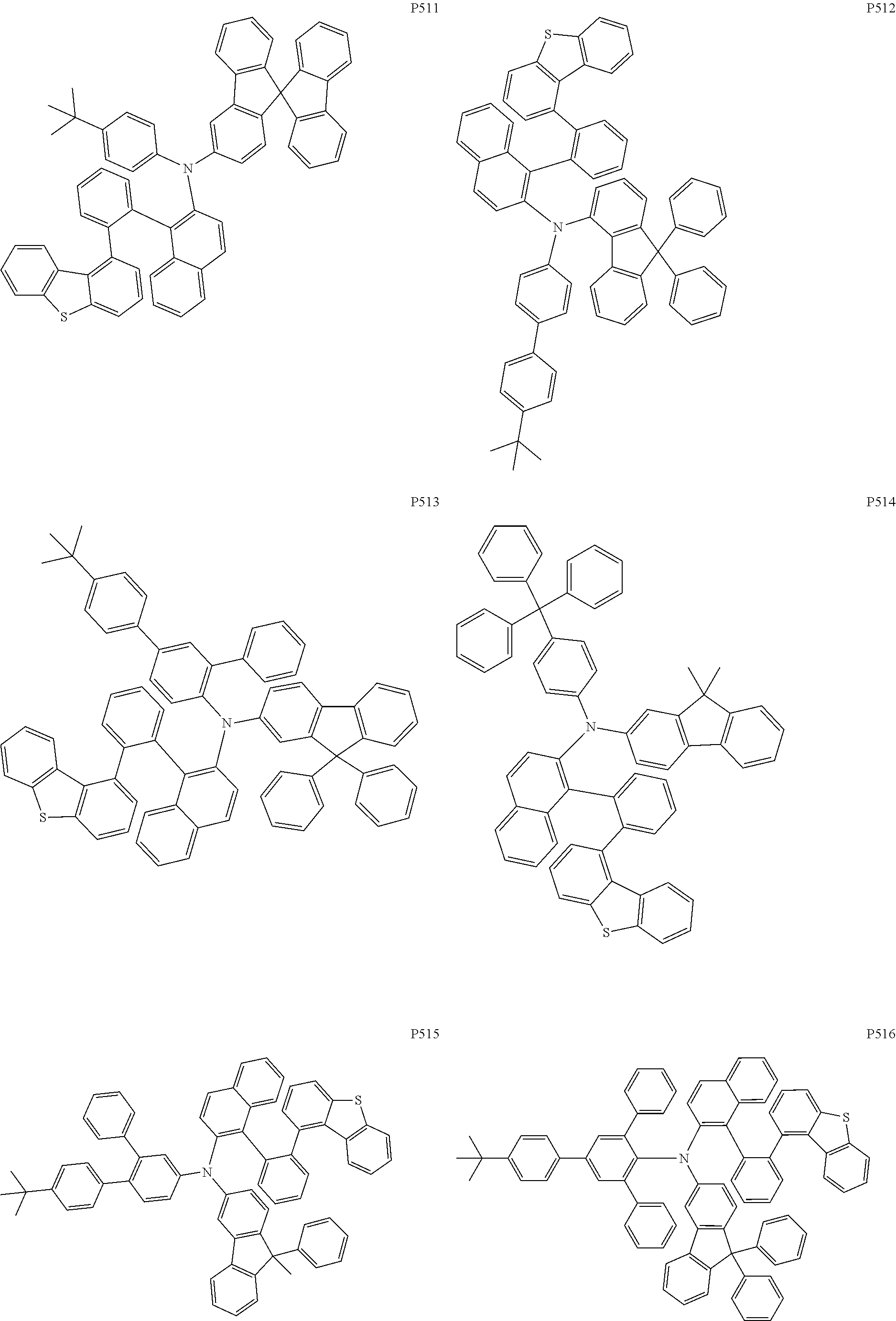

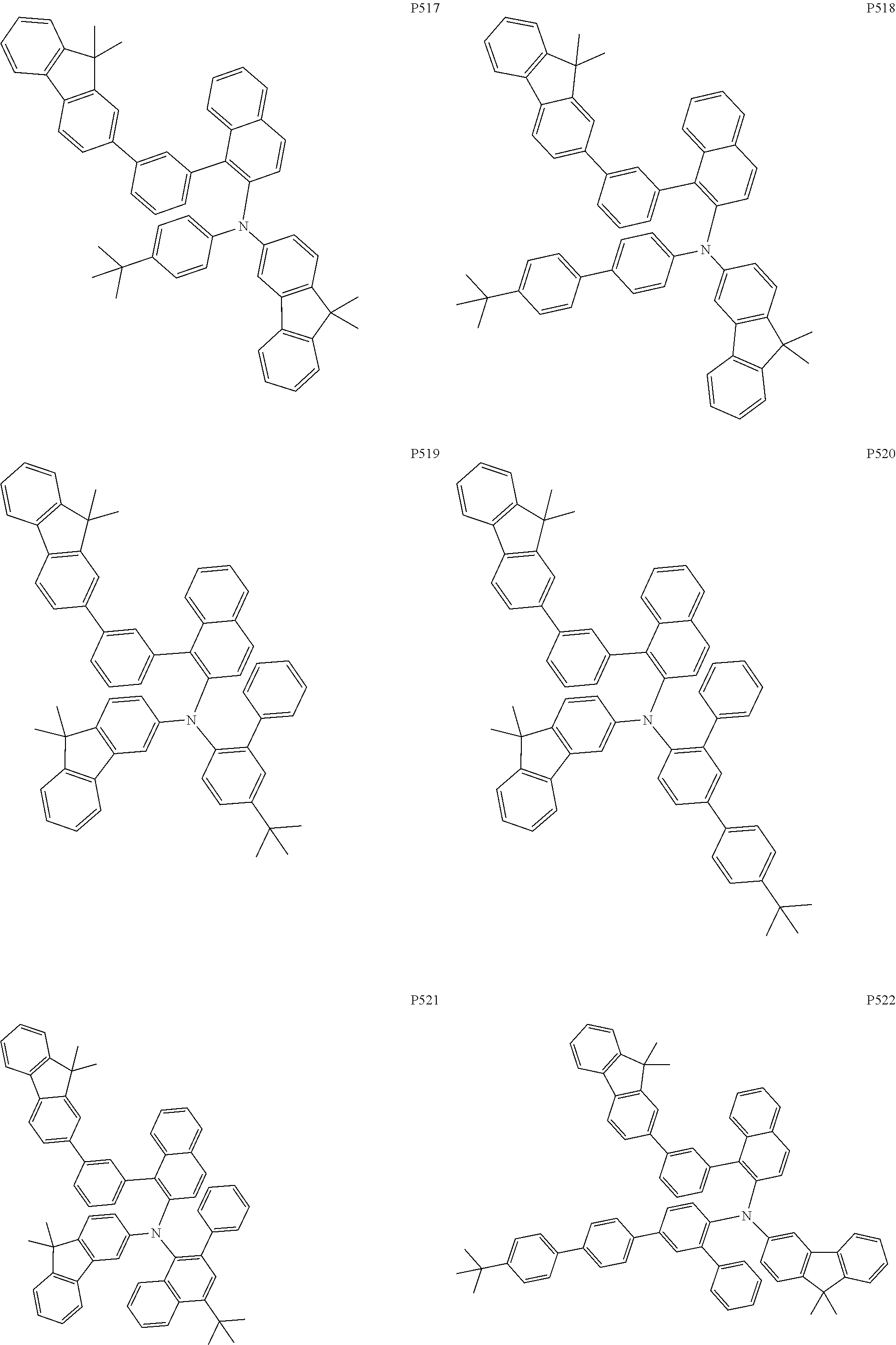

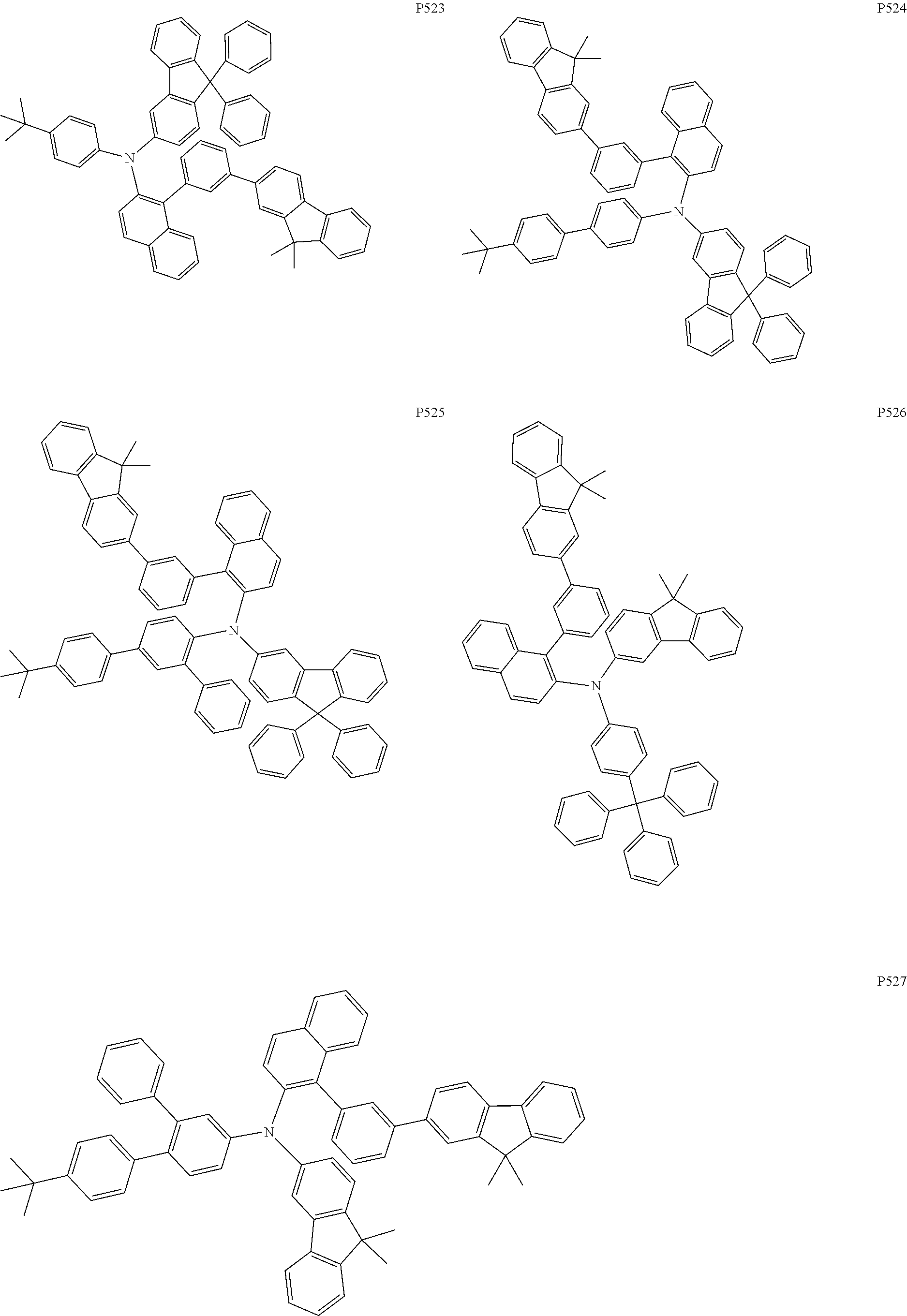

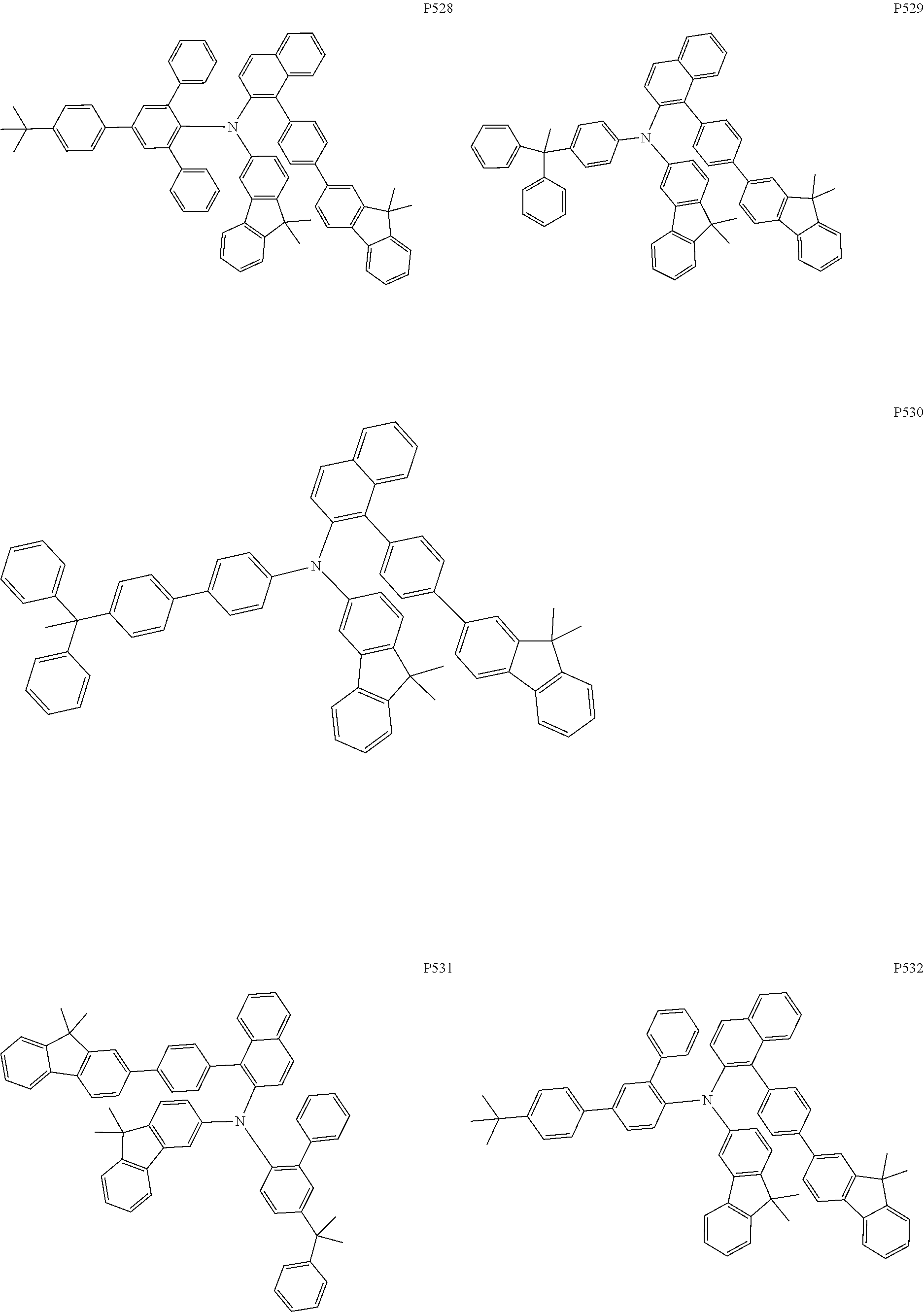

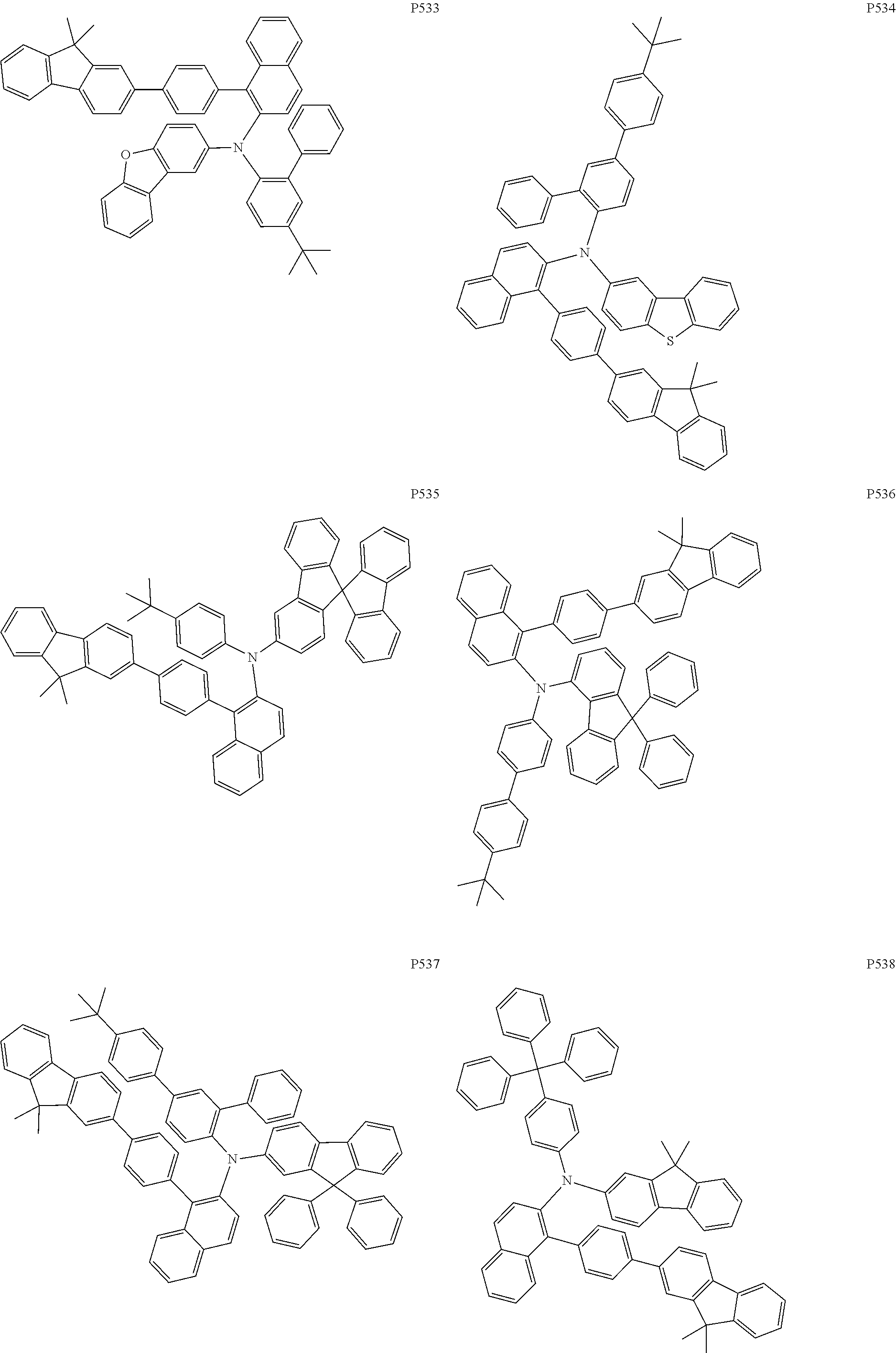

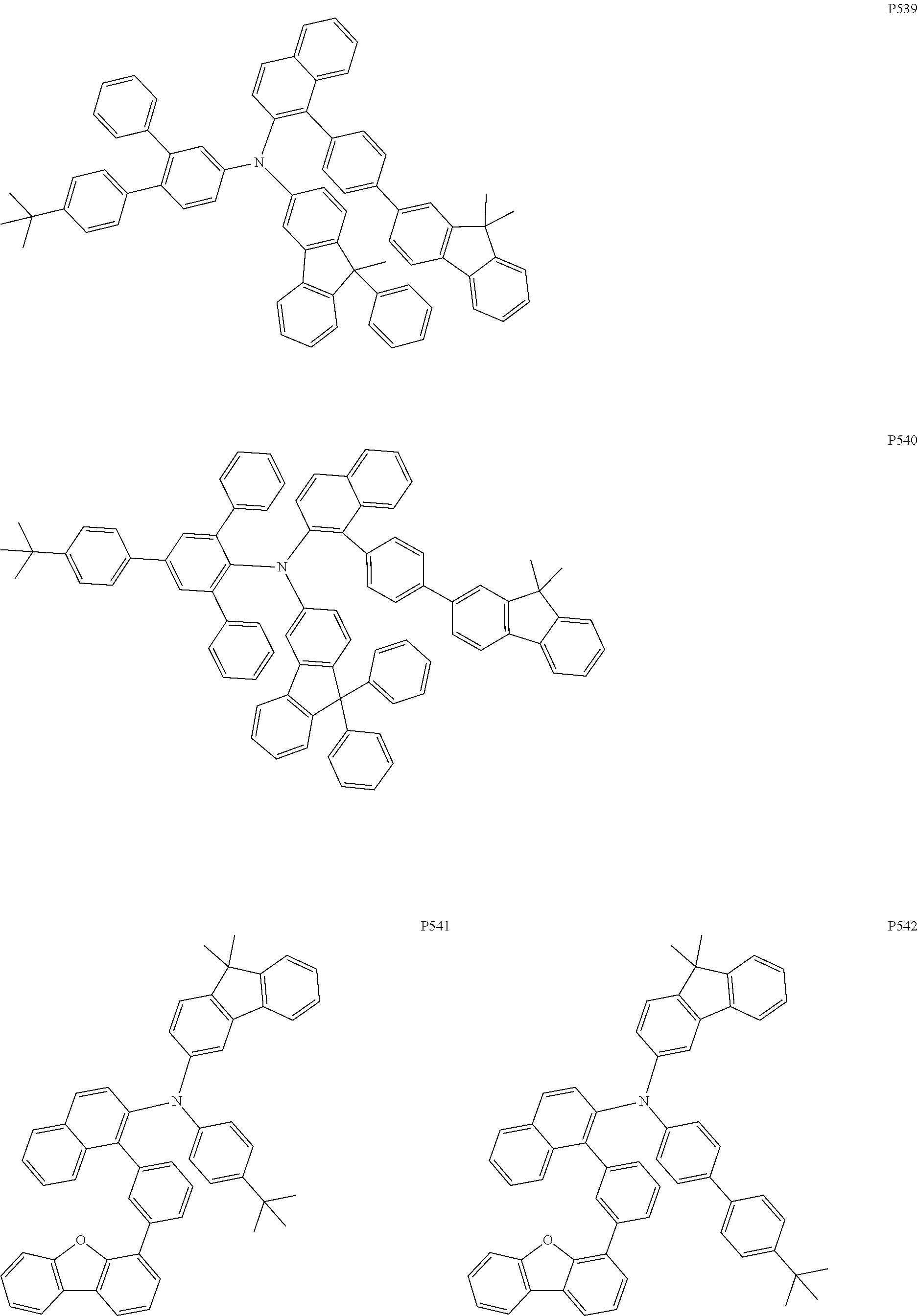 [SMILES: CC(C)(C)c1ccc(-c2ccc(N(c3ccc4c(c3)-c3ccccc3C4(C)c3ccccc3)c3ccc4ccccc4c3-c3ccc(-c4ccc5c(c4)C(C)(C)c4ccccc4-5)cc3)cc2-c2ccccc2)cc1 ; CC(C)(C)c1ccc(-c2cc(-c3ccccc3)c(N(c3ccc4c(c3)-c3ccccc3C4(c3ccccc3)c3ccccc3)c3ccc4ccccc4c3-c3ccc(-c4ccc5c(c4)C(C)(C)c4ccccc4-5)cc3)c(-c3ccccc3)c2)cc1 ; CC(C)(C)c1ccc(N(c2ccc3c(c2)-c2ccccc2C3(C)C)c2ccc3ccccc3c2-c2cccc(-c3cccc4c3oc3ccccc34)c2)cc1 ; CC(C)(C)c1ccc(-c2ccc(N(c3ccc4c(c3)-c3ccccc3C4(C)C)c3ccc4ccccc4c3-c3cccc(-c4cccc5c4oc4ccccc45)c3)cc2)cc1]

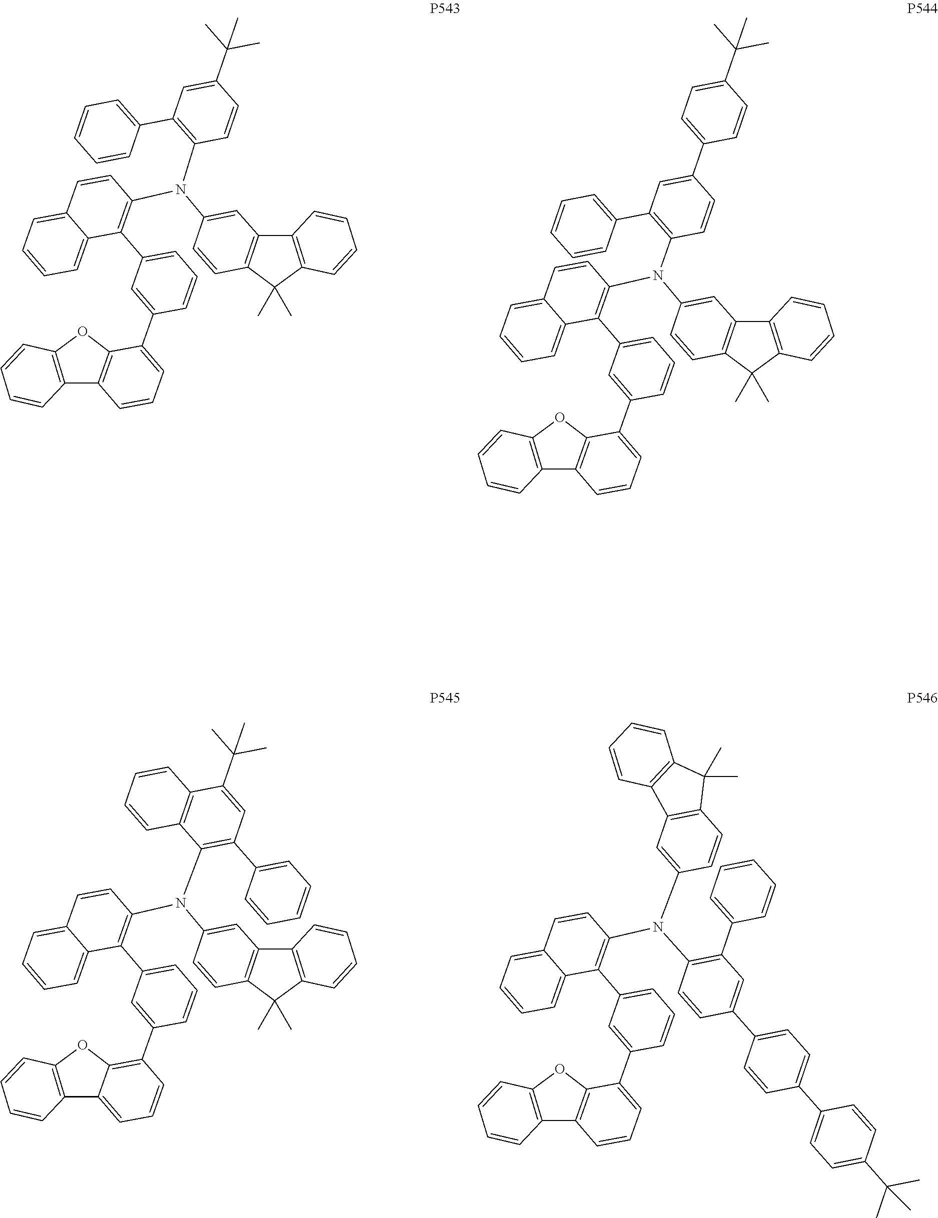 [SMILES: CC(C)(C)c1ccc(N(c2ccc3c(c2)-c2ccccc2C3(C)C)c2ccc3ccccc3c2-c2cccc(-c3cccc4c3oc3ccccc34)c2)c(-c2ccccc2)c1 ; CC(C)(C)c1ccc(-c2ccc(N(c3ccc4c(c3)-c3ccccc3C4(C)C)c3ccc4ccccc4c3-c3cccc(-c4cccc5c4oc4ccccc45)c3)c(-c3ccccc3)c2)cc1 ; CC(C)(C)c1cc(-c2ccccc2)c(N(c2ccc3c(c2)-c2ccccc2C3(C)C)c2ccc3ccccc3c2-c2cccc(-c3cccc4c3oc3ccccc34)c2)c2ccccc12 ; CC(C)(C)c1ccc(-c2ccc(-c3ccc(N(c4ccc5c(c4)-c4ccccc4C5(C)C)c4ccc5ccccc5c4-c4cccc(-c5cccc6c5oc5ccccc56)c4)c(-c4ccccc4)c3)cc2)cc1]

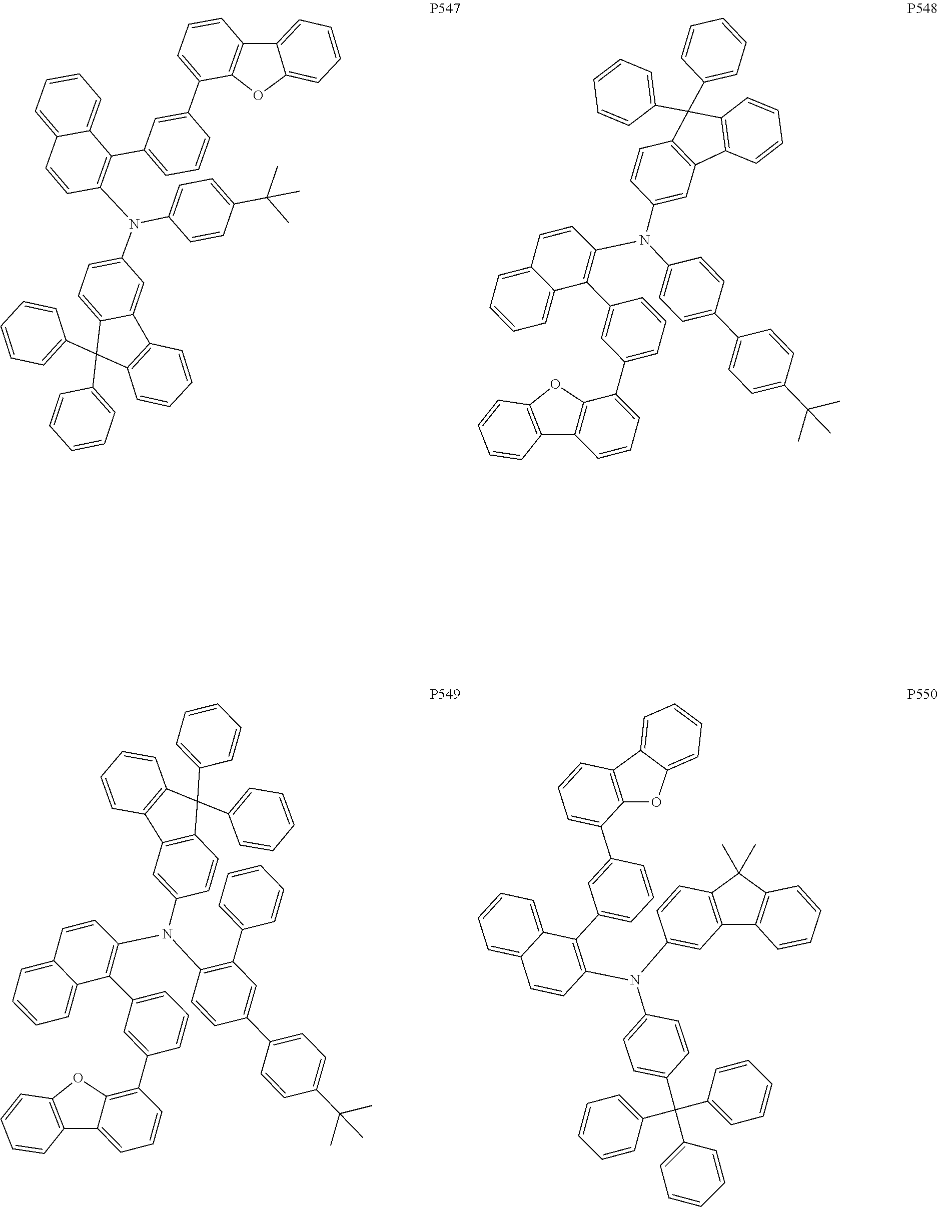 [SMILES: CC(C)(C)c1ccc(N(c2ccc3c(c2)-c2ccccc2C3(c2ccccc2)c2ccccc2)c2ccc3ccccc3c2-c2cccc(-c3cccc4c3oc3ccccc34)c2)cc1 ; CC(C)(C)c1ccc(-c2ccc(N(c3ccc4c(c3)-c3ccccc3C4(c3ccccc3)c3ccccc3)c3ccc4ccccc4c3-c3cccc(-c4cccc5c4oc4ccccc45)c3)cc2)cc1 ; CC(C)(C)c1ccc(-c2ccc(N(c3ccc4c(c3)-c3ccccc3C4(c3ccccc3)c3ccccc3)c3ccc4ccccc4c3-c3cccc(-c4cccc5c4oc4ccccc45)c3)c(-c3ccccc3)c2)cc1 ; CC1(C)c2ccccc2-c2cc(N(c3ccc(C(c4ccccc4)(c4ccccc4)c4ccccc4)cc3)c3ccc4ccccc4c3-c3cccc(-c4cccc5c4oc4ccccc45)c3)ccc21]

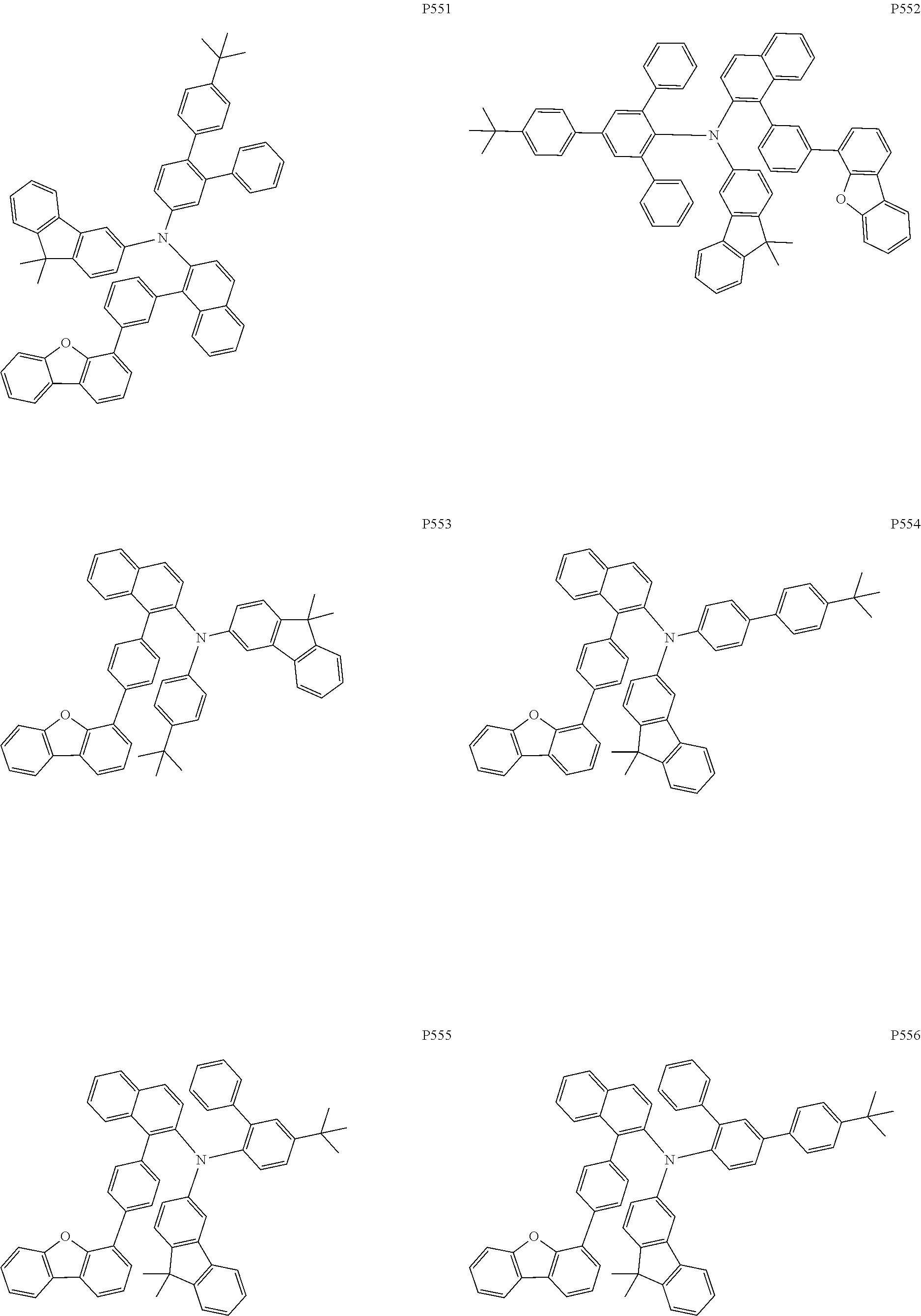

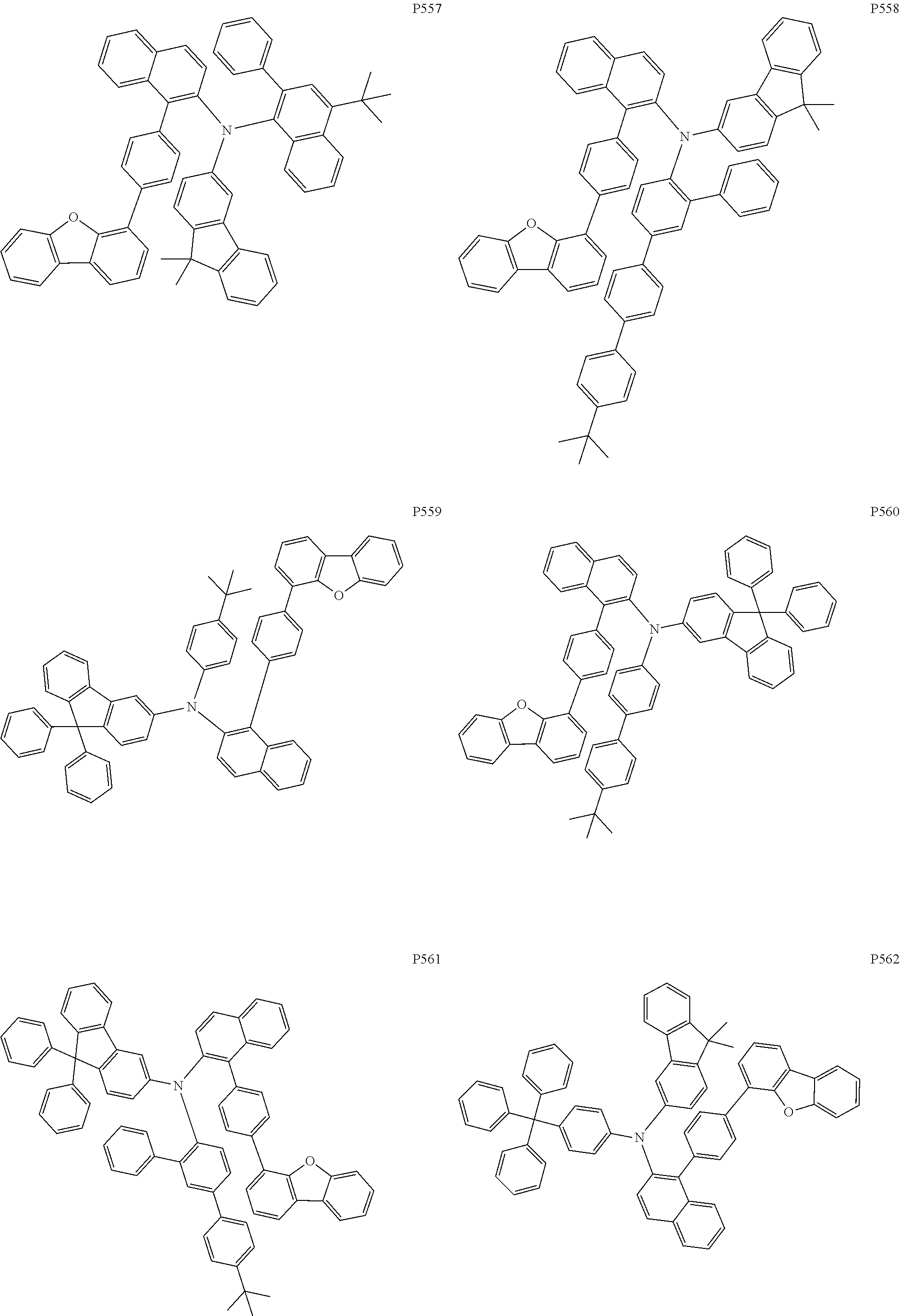

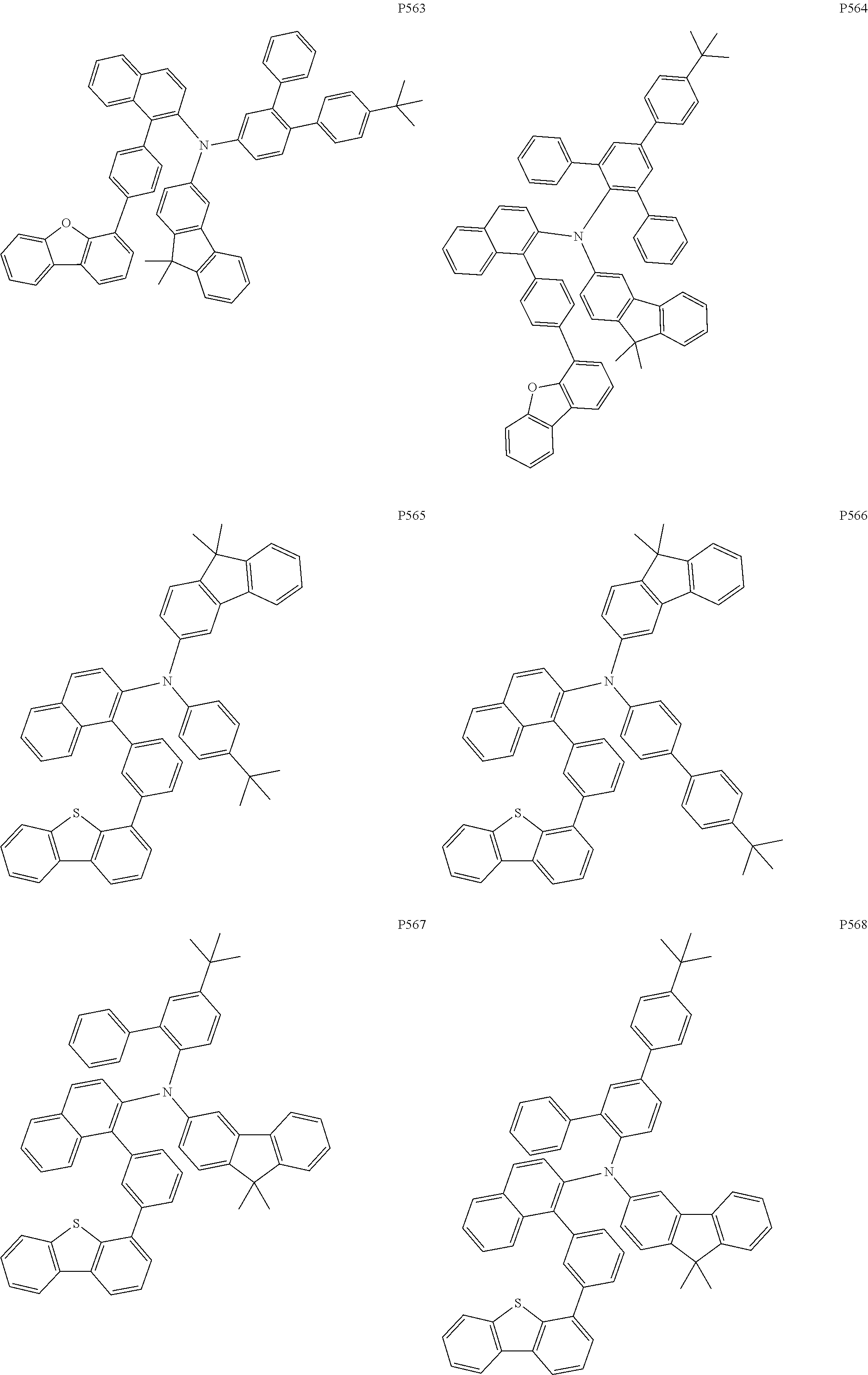

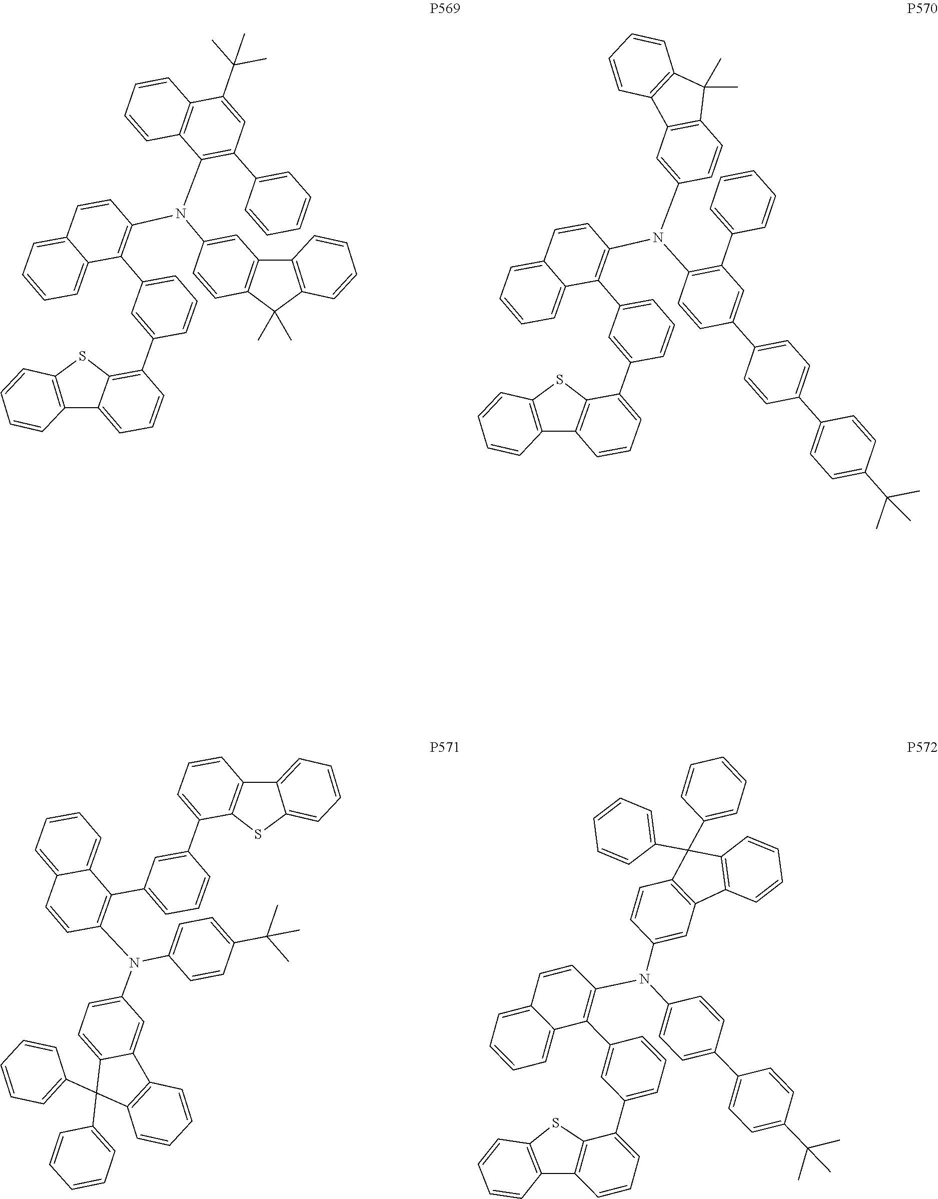 [SMILES: CC(C)(C)c1cc(-c2ccccc2)c(N(c2ccc3c(c2)-c2ccccc2C3(C)C)c2ccc3ccccc3c2-c2cccc(-c3cccc4c3sc3ccccc34)c2)c2ccccc12 ; CC(C)(C)c1ccc(-c2ccc(-c3ccc(N(c4ccc5c(c4)-c4ccccc4C5(C)C)c4ccc5ccccc5c4-c4cccc(-c5cccc6c5sc5ccccc56)c4)c(-c4ccccc4)c3)cc2)cc1 ; CC(C)(C)c1ccc(N(c2ccc3c(c2)-c2ccccc2C3(c2ccccc2)c2ccccc2)c2ccc3ccccc3c2-c2cccc(-c3cccc4c3sc3ccccc34)c2)cc1 ; CC(C)(C)c1ccc(-c2ccc(N(c3ccc4c(c3)-c3ccccc3C4(c3ccccc3)c3ccccc3)c3ccc4ccccc4c3-c3cccc(-c4cccc5c4sc4ccccc45)c3)cc2)cc1]

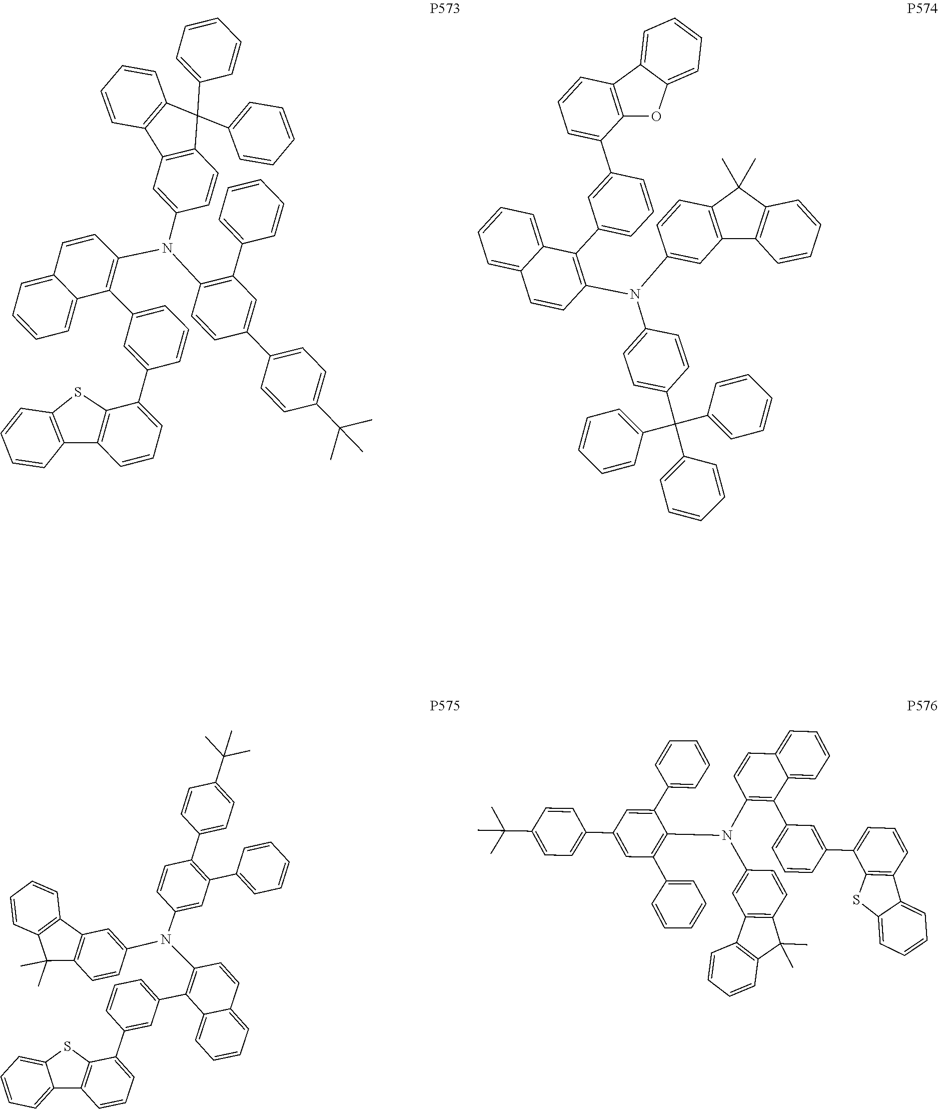 [SMILES: CC(C)(C)c1ccc(-c2ccc(N(c3ccc4c(c3)-c3ccccc3C4(c3ccccc3)c3ccccc3)c3ccc4ccccc4c3-c3cccc(-c4cccc5c4sc4ccccc45)c3)c(-c3ccccc3)c2)cc1 ; CC1(C)c2ccccc2-c2cc(N(c3ccc(C(c4ccccc4)(c4ccccc4)c4ccccc4)cc3)c3ccc4ccccc4c3-c3cccc(-c4cccc5c4oc4ccccc45)c3)ccc21 ; CC(C)(C)c1ccc(-c2ccc(N(c3ccc4c(c3)-c3ccccc3C4(C)C)c3ccc4ccccc4c3-c3cccc(-c4cccc5c4sc4ccccc45)c3)cc2-c2ccccc2)cc1 ; CC(C)(C)c1ccc(-c2cc(-c3ccccc3)c(N(c3ccc4c(c3)-c3ccccc3C4(C)C)c3ccc4ccccc4c3-c3cccc(-c4cccc5c4sc4ccccc45)c3)c(-c3ccccc3)c2)cc1]

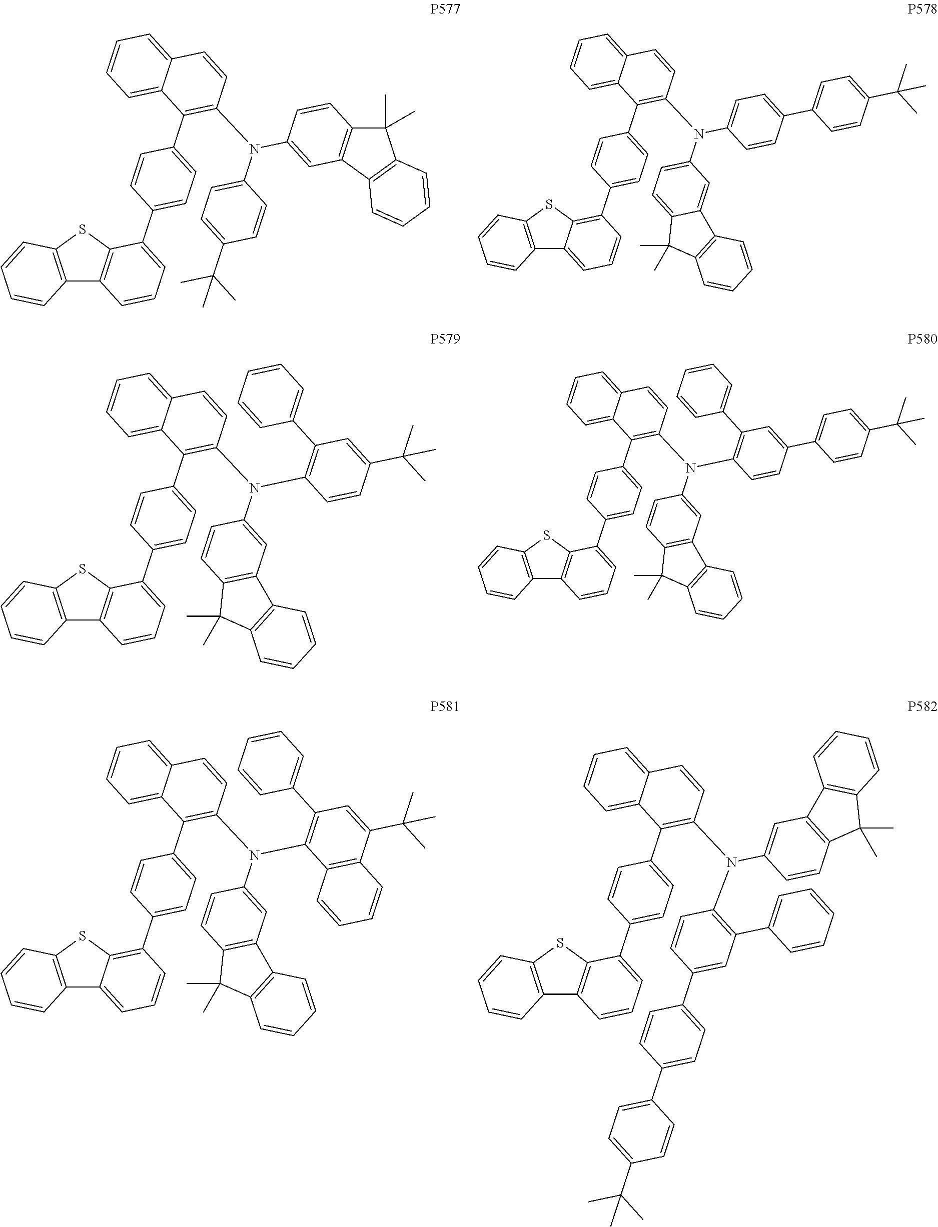

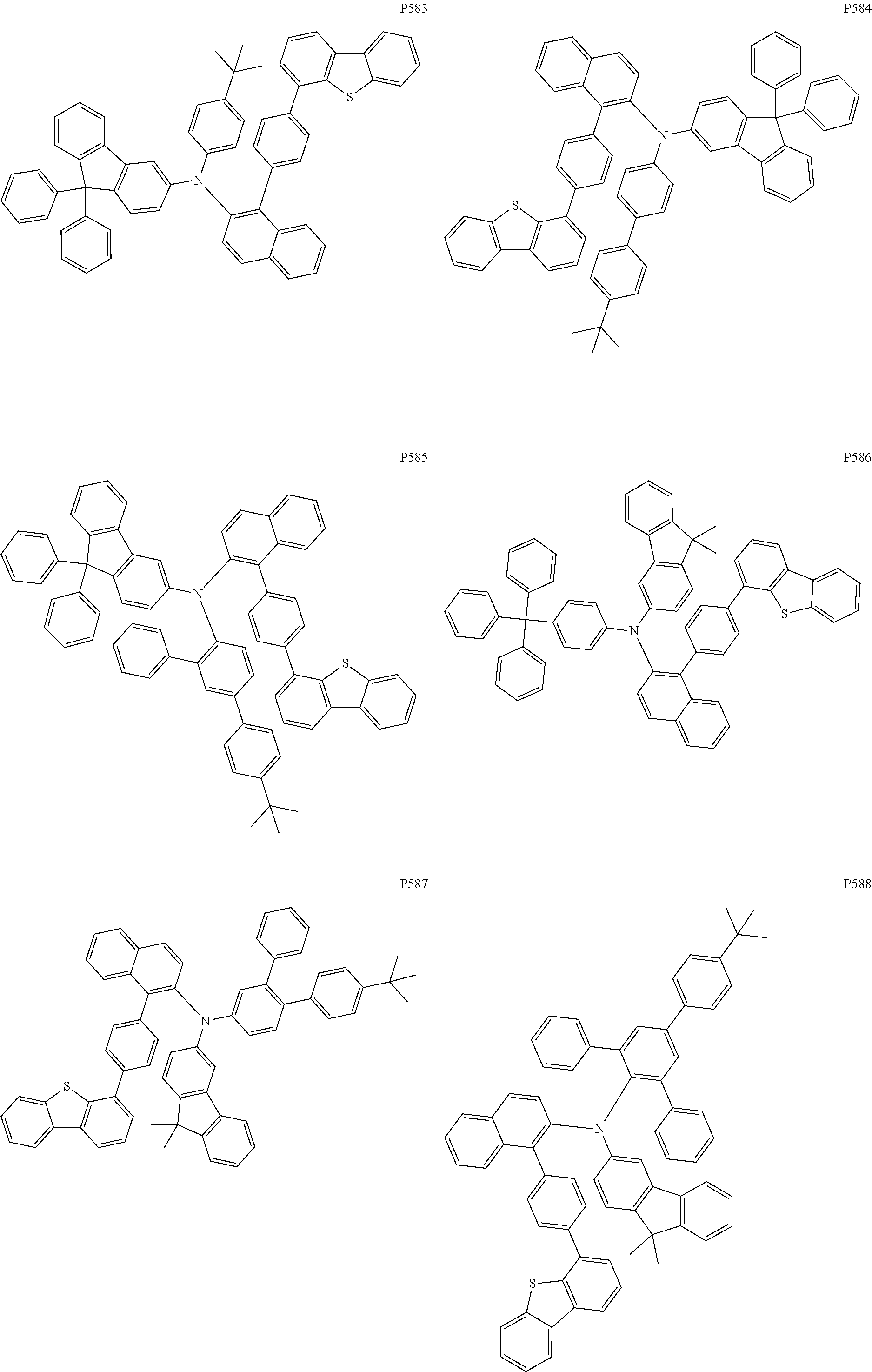

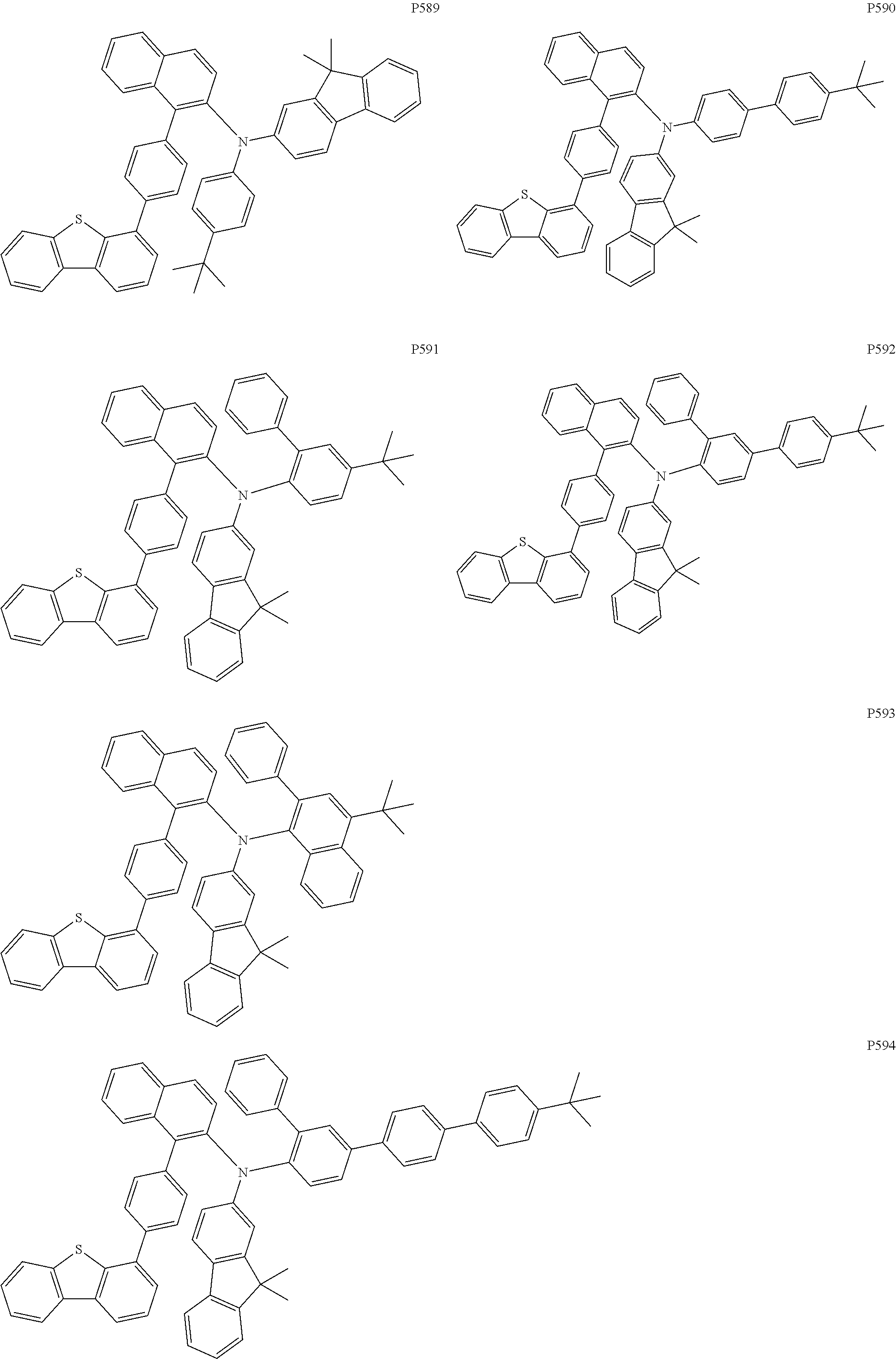

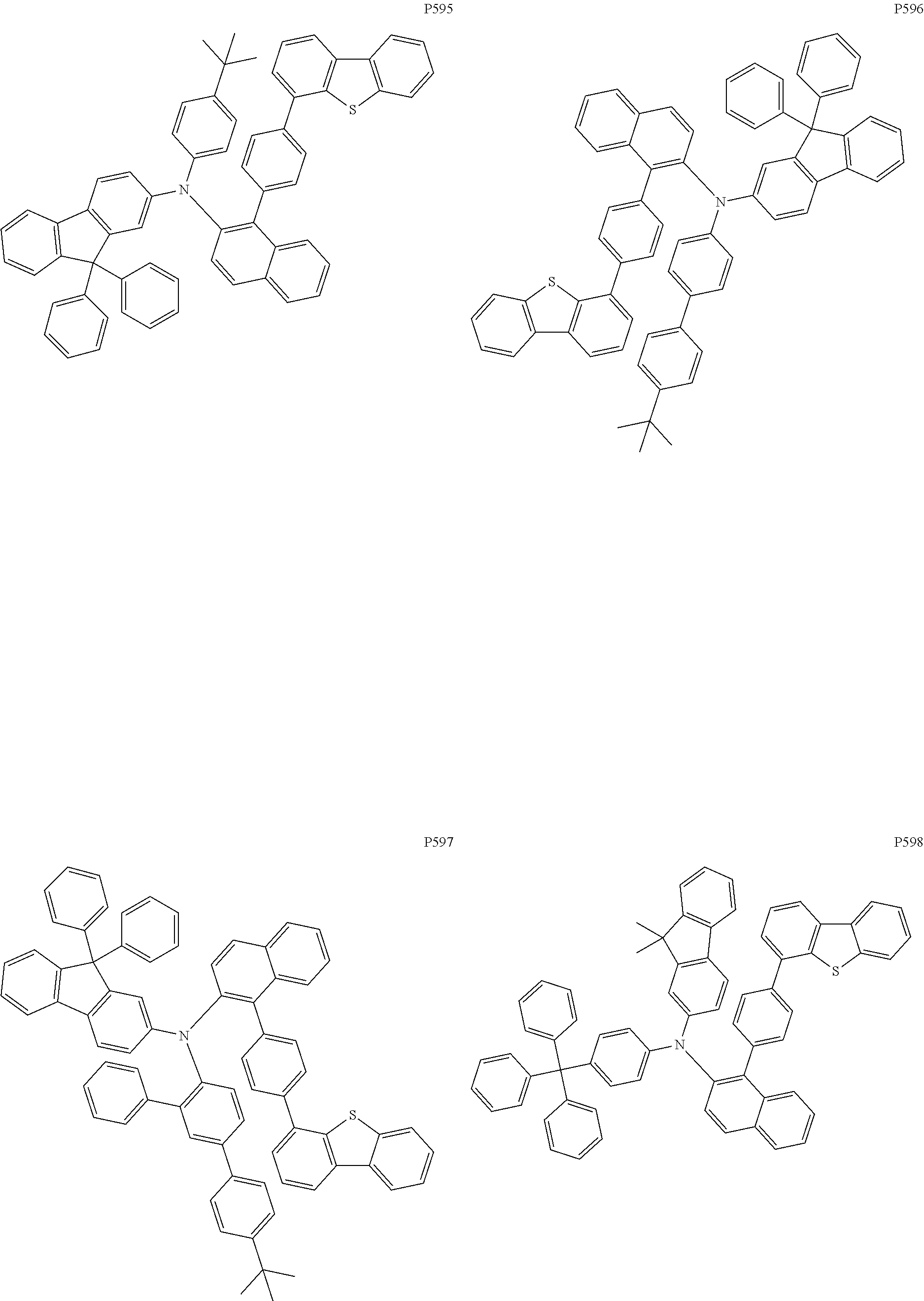 [SMILES: CC(C)(C)c1ccc(N(c2ccc3c(c2)C(c2ccccc2)(c2ccccc2)c2ccccc2-3)c2ccc3ccccc3c2-c2ccc(-c3cccc4c3sc3ccccc34)cc2)cc1 ; CC(C)(C)c1ccc(-c2ccc(N(c3ccc4c(c3)C(c3ccccc3)(c3ccccc3)c3ccccc3-4)c3ccc4ccccc4c3-c3ccc(-c4cccc5c4sc4ccccc45)cc3)cc2)cc1 ; CC(C)(C)c1ccc(-c2ccc(N(c3ccc4c(c3)C(c3ccccc3)(c3ccccc3)c3ccccc3-4)c3ccc4ccccc4c3-c3ccc(-c4cccc5c4sc4ccccc45)cc3)c(-c3ccccc3)c2)cc1 ; CC1(C)c2ccccc2-c2ccc(N(c3ccc(C(c4ccccc4)(c4ccccc4)c4ccccc4)cc3)c3ccc4ccccc4c3-c3ccc(-c4cccc5c4sc4ccccc45)cc3)cc21]

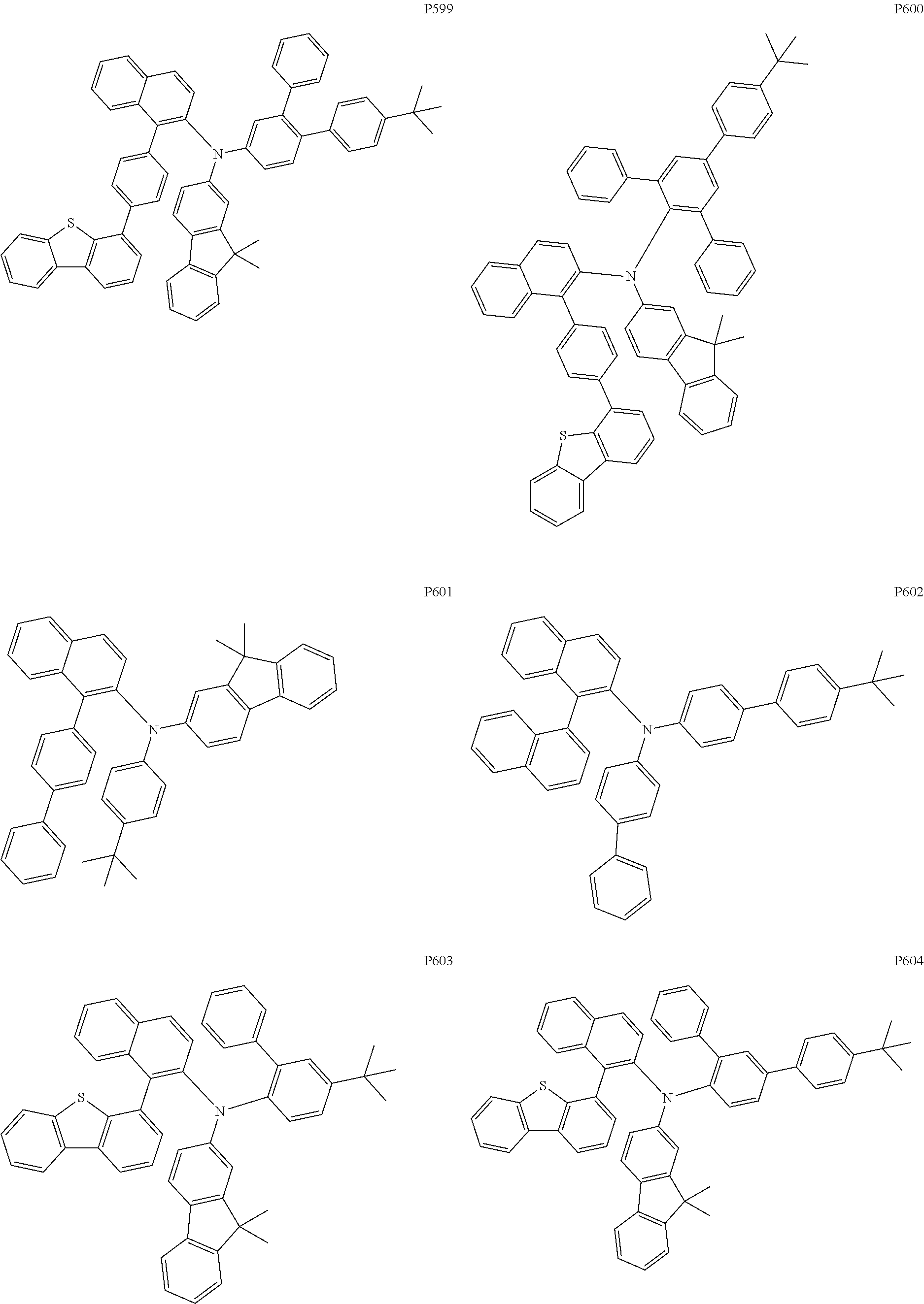

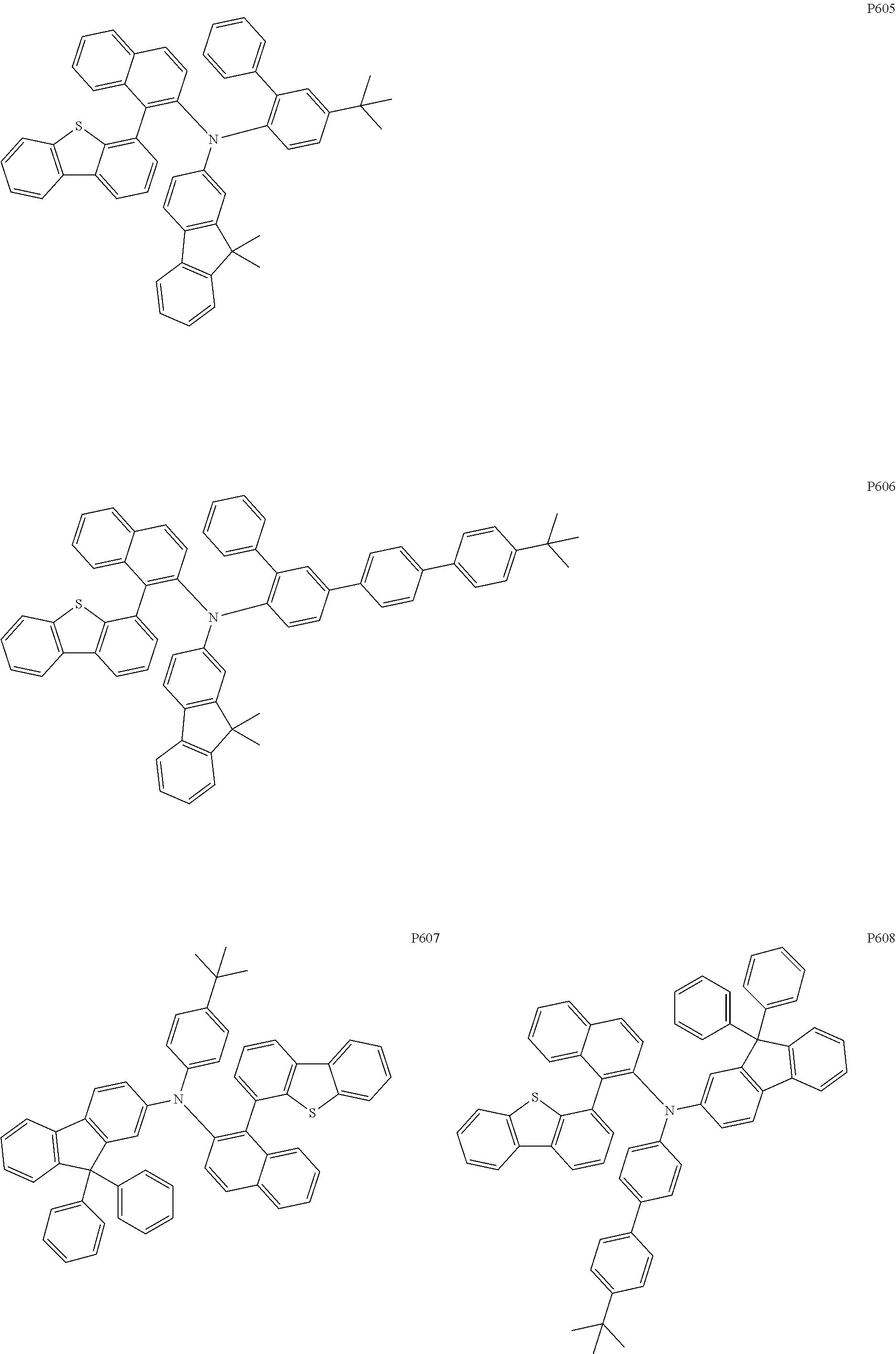 [SMILES: CC(C)(C)c1ccc(N(c2ccc3c(c2)C(C)(C)c2ccccc2-3)c2ccc3ccccc3c2-c2cccc3c2sc2ccccc23)c(-c2ccccc2)c1 ; CC(C)(C)c1ccc(-c2ccc(-c3ccc(N(c4ccc5c(c4)C(C)(C)c4ccccc4-5)c4ccc5ccccc5c4-c4cccc5c4sc4ccccc45)c(-c4ccccc4)c3)cc2)cc1 ; CC(C)(C)c1ccc(N(c2ccc3c(c2)C(c2ccccc2)(c2ccccc2)c2ccccc2-3)c2ccc3ccccc3c2-c2cccc3c2sc2ccccc23)cc1 ; CC(C)(C)c1ccc(-c2ccc(N(c3ccc4c(c3)C(c3ccccc3)(c3ccccc3)c3ccccc3-4)c3ccc4ccccc4c3-c3cccc4c3sc3ccccc34)cc2)cc1]

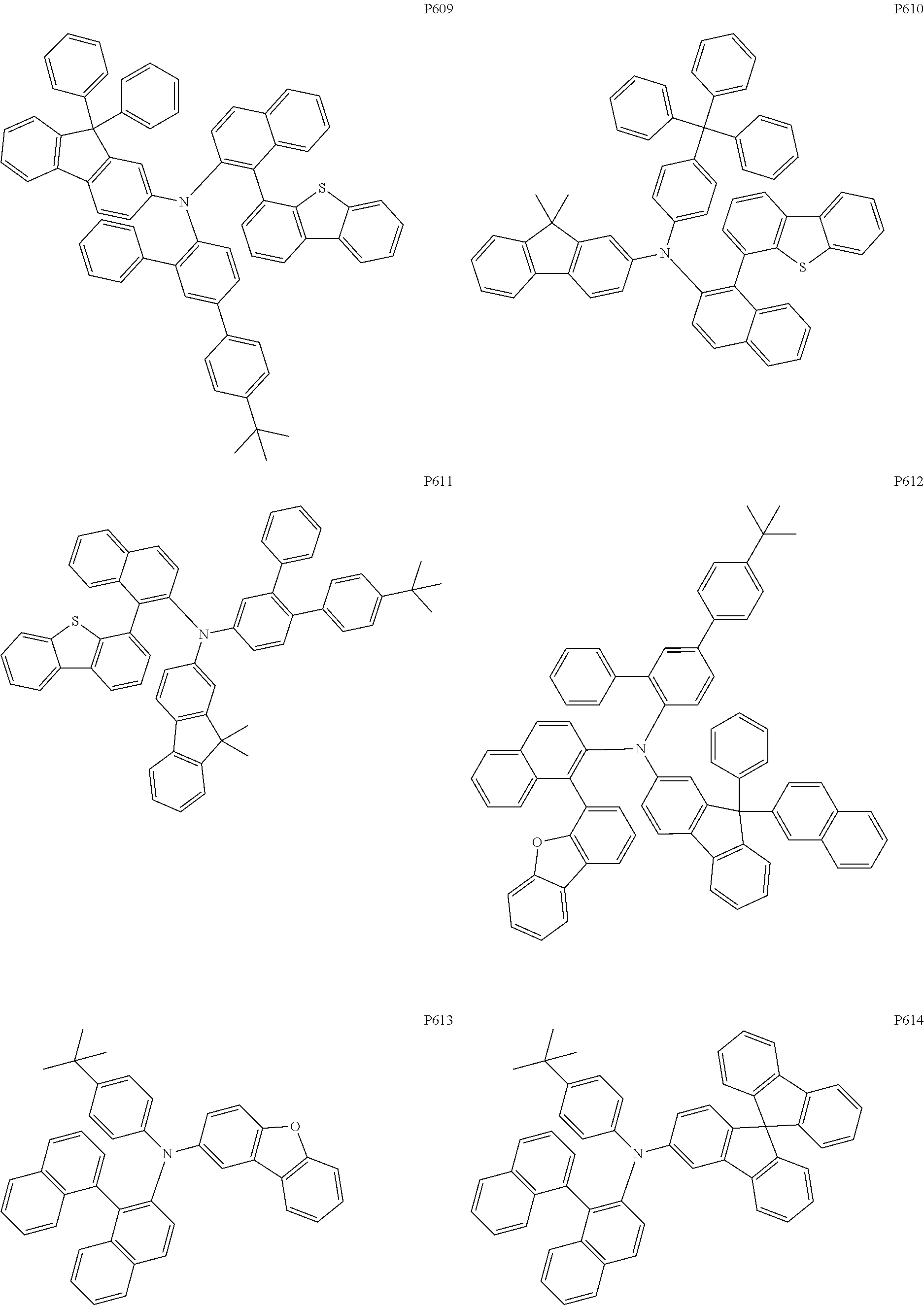

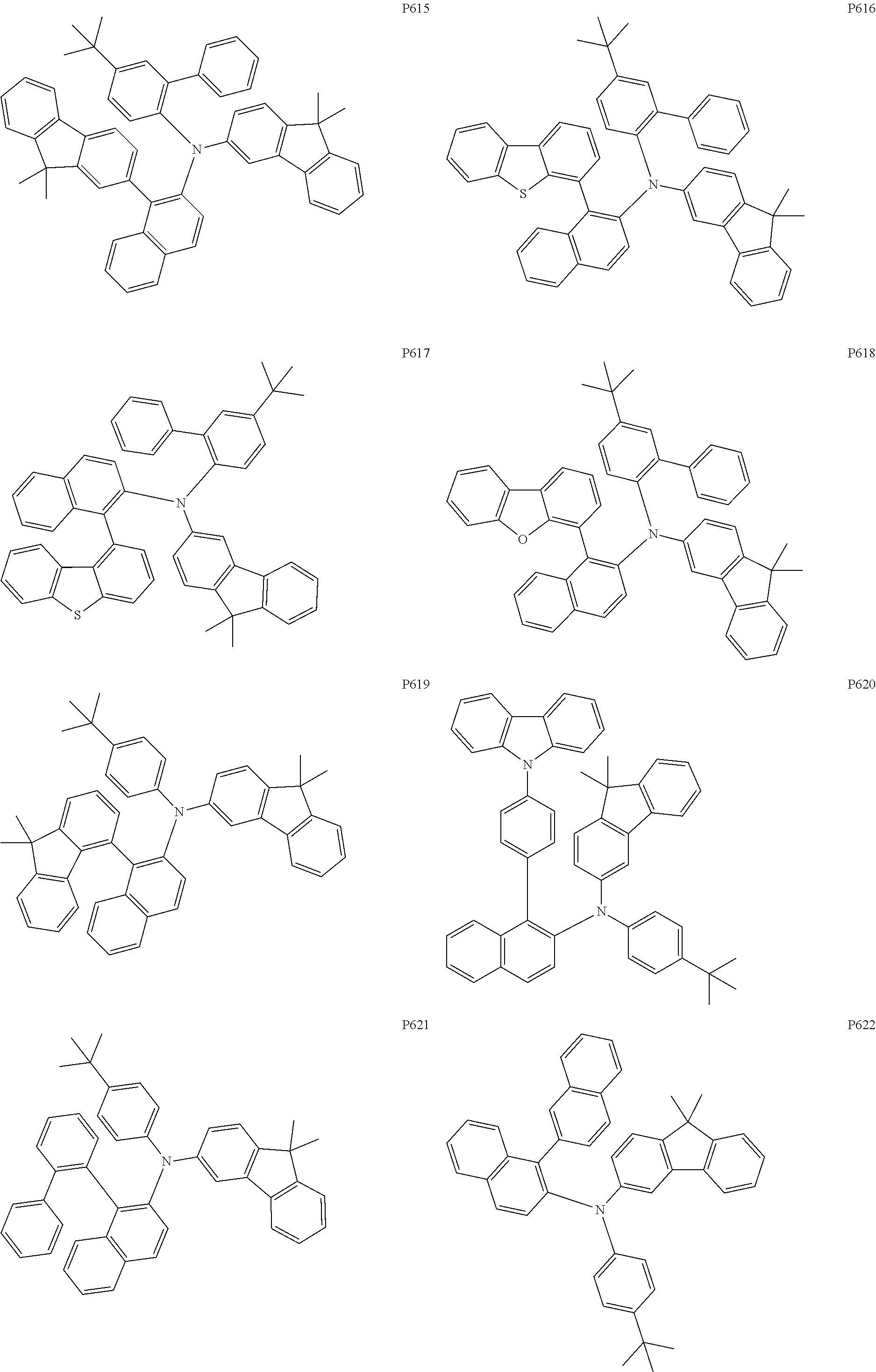

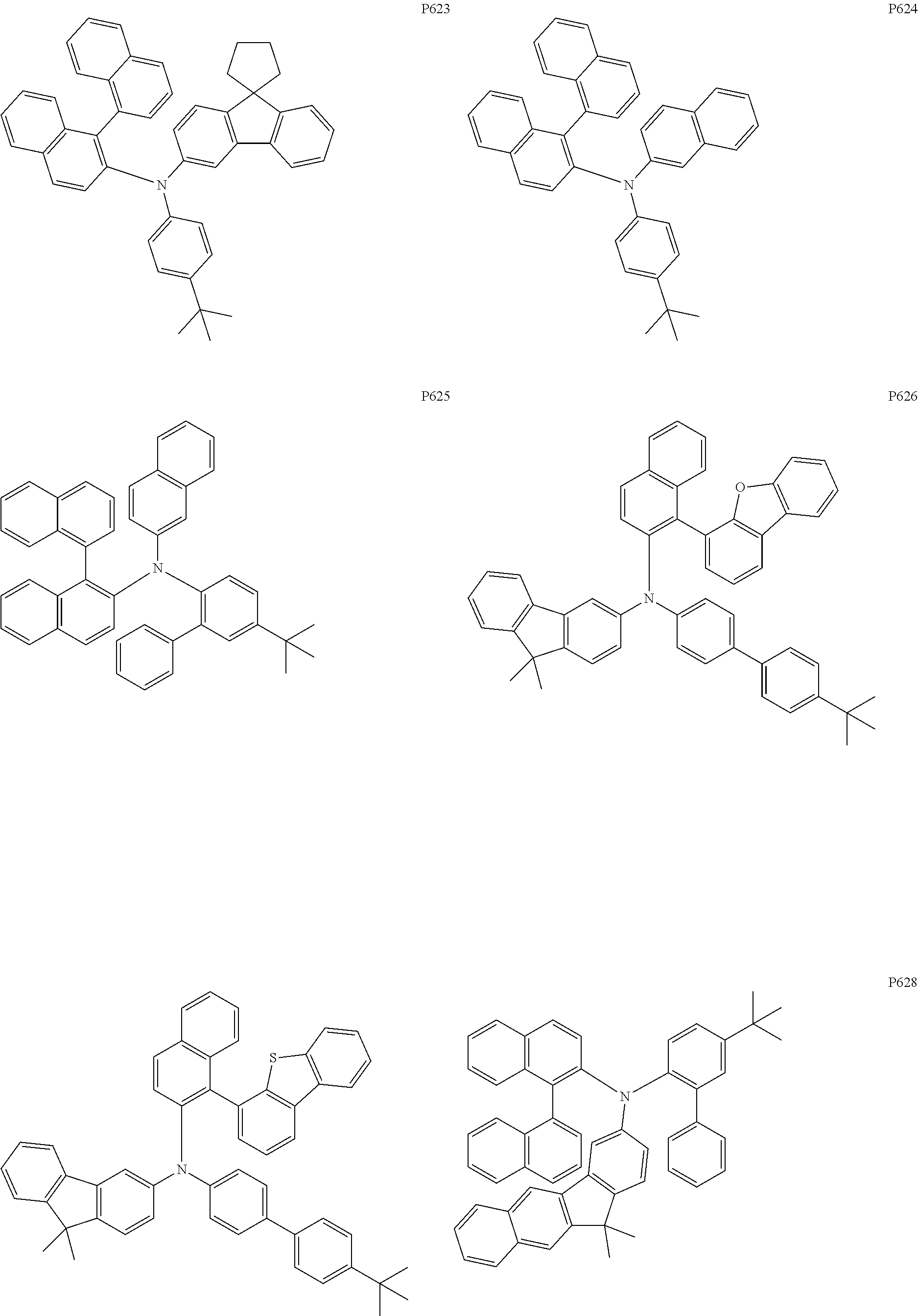

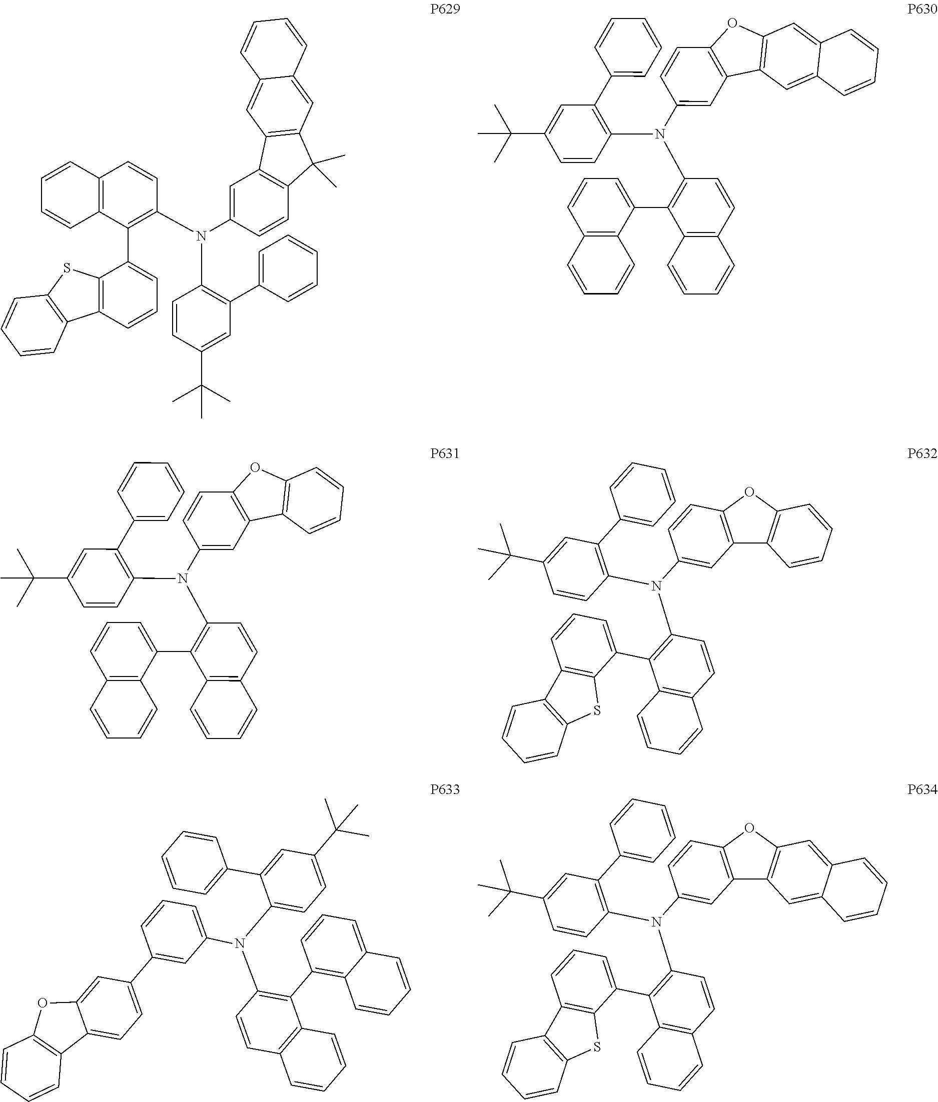

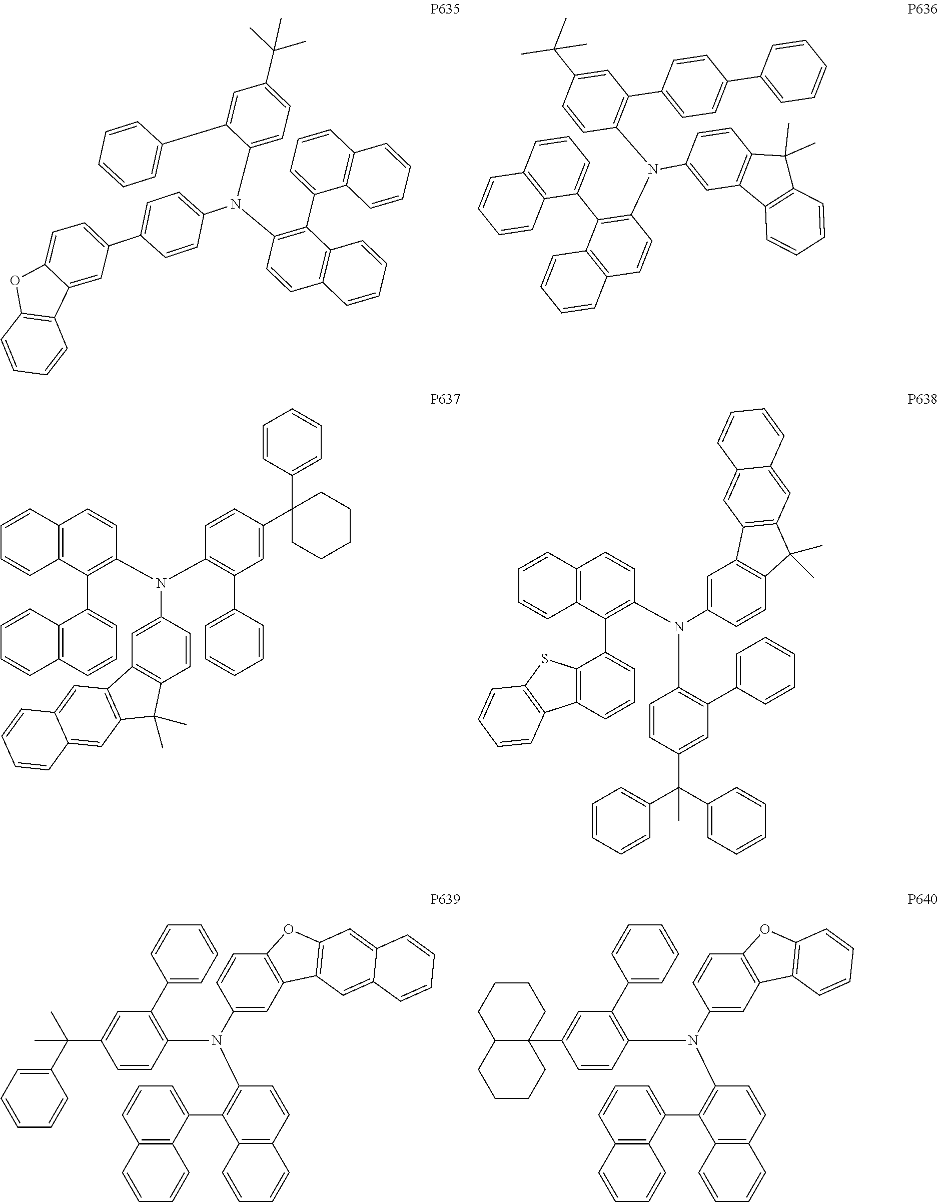

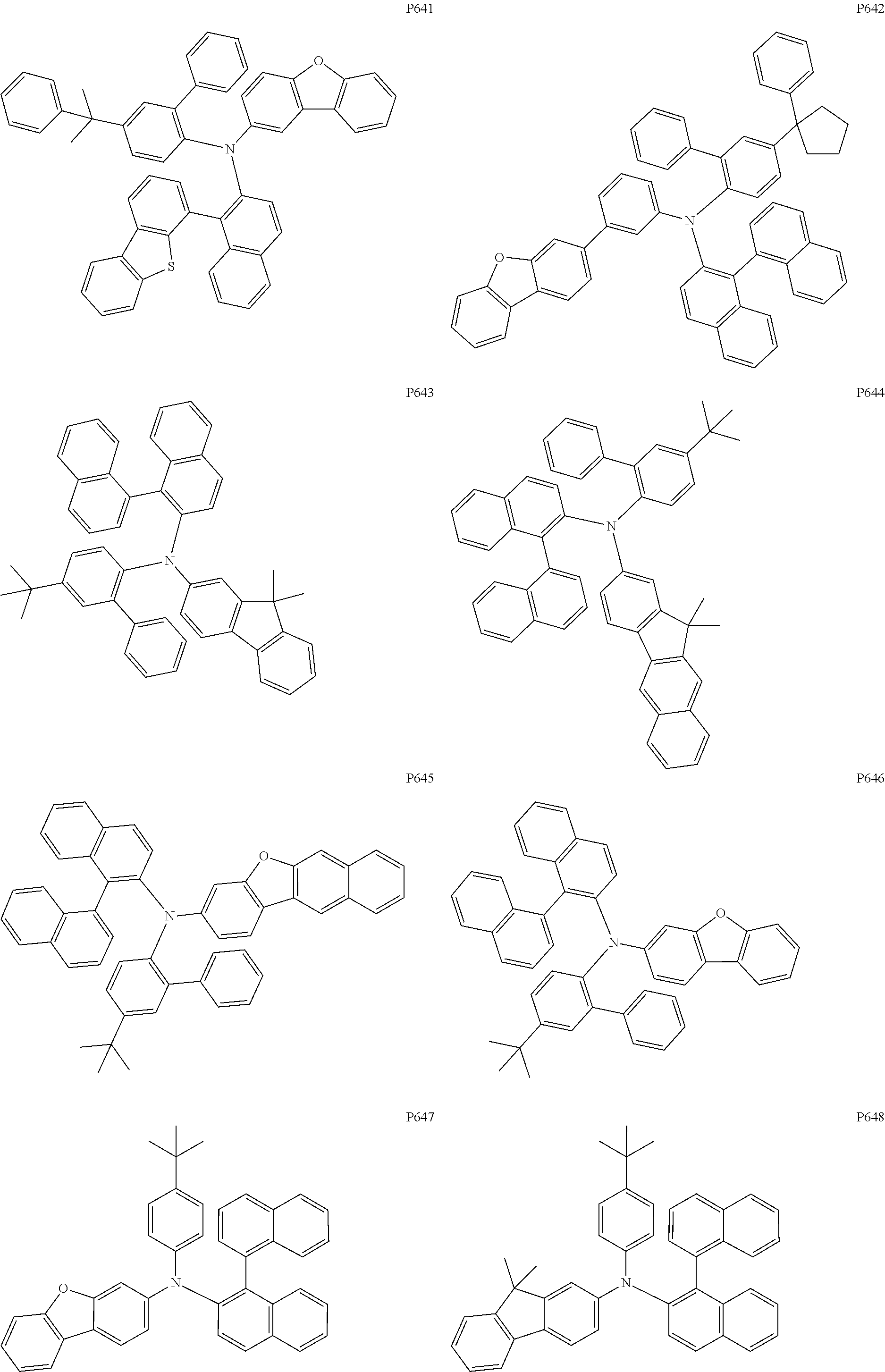

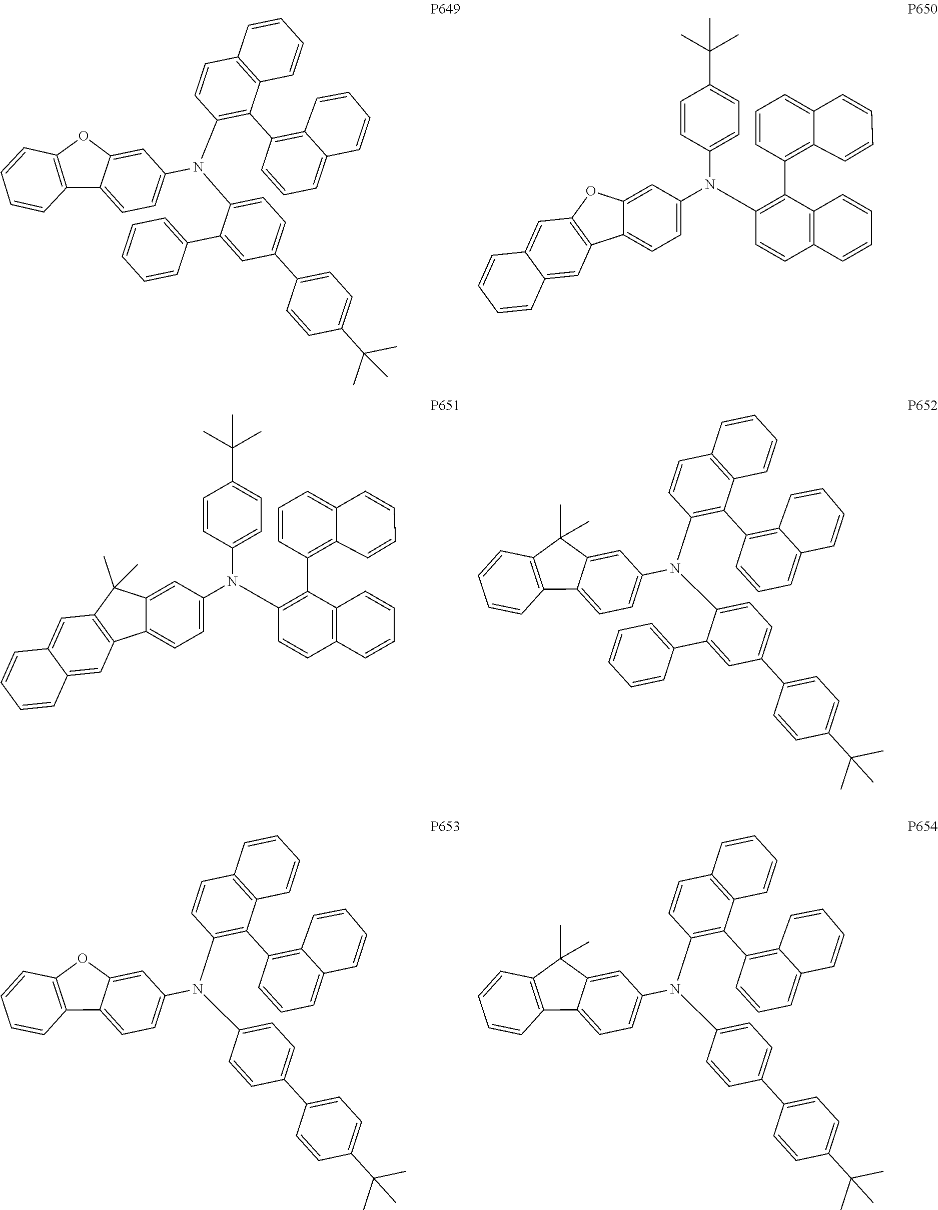

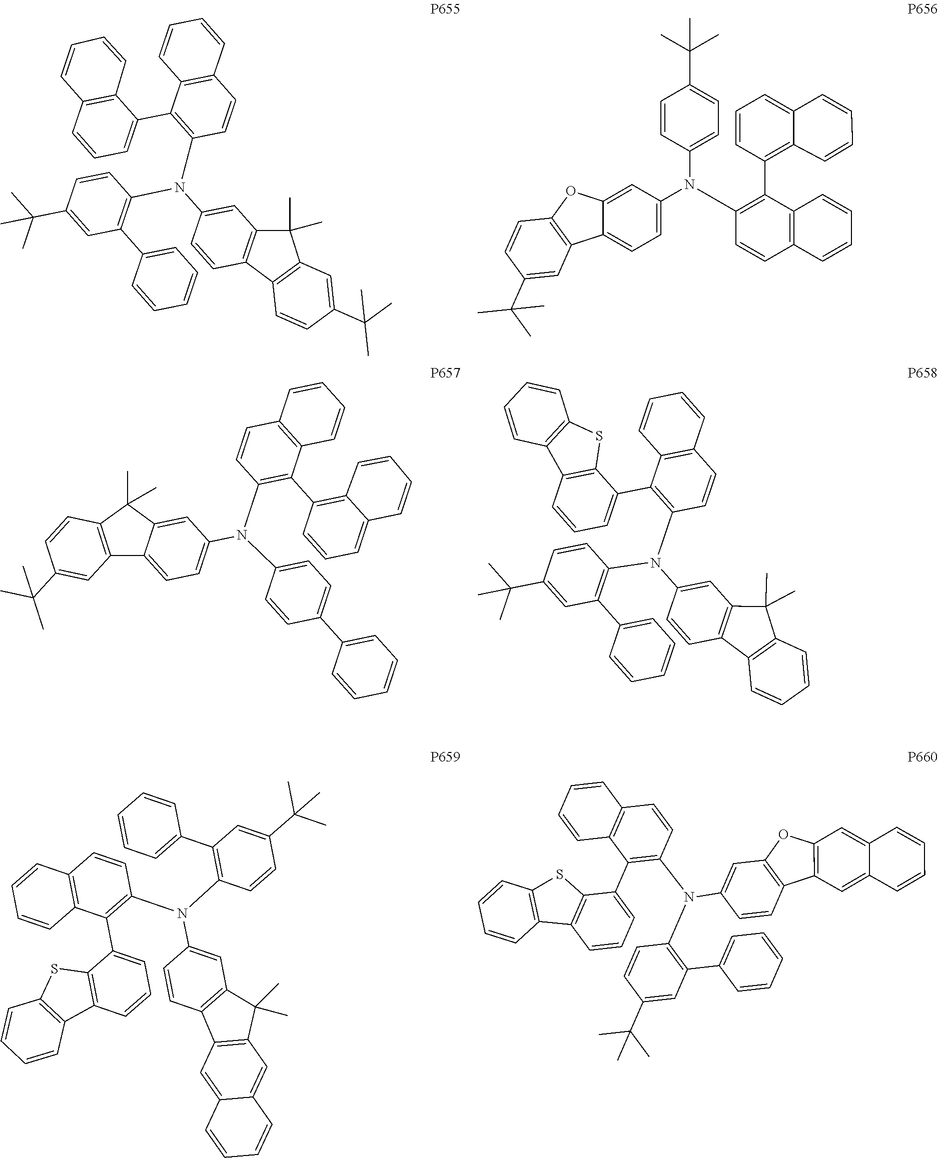

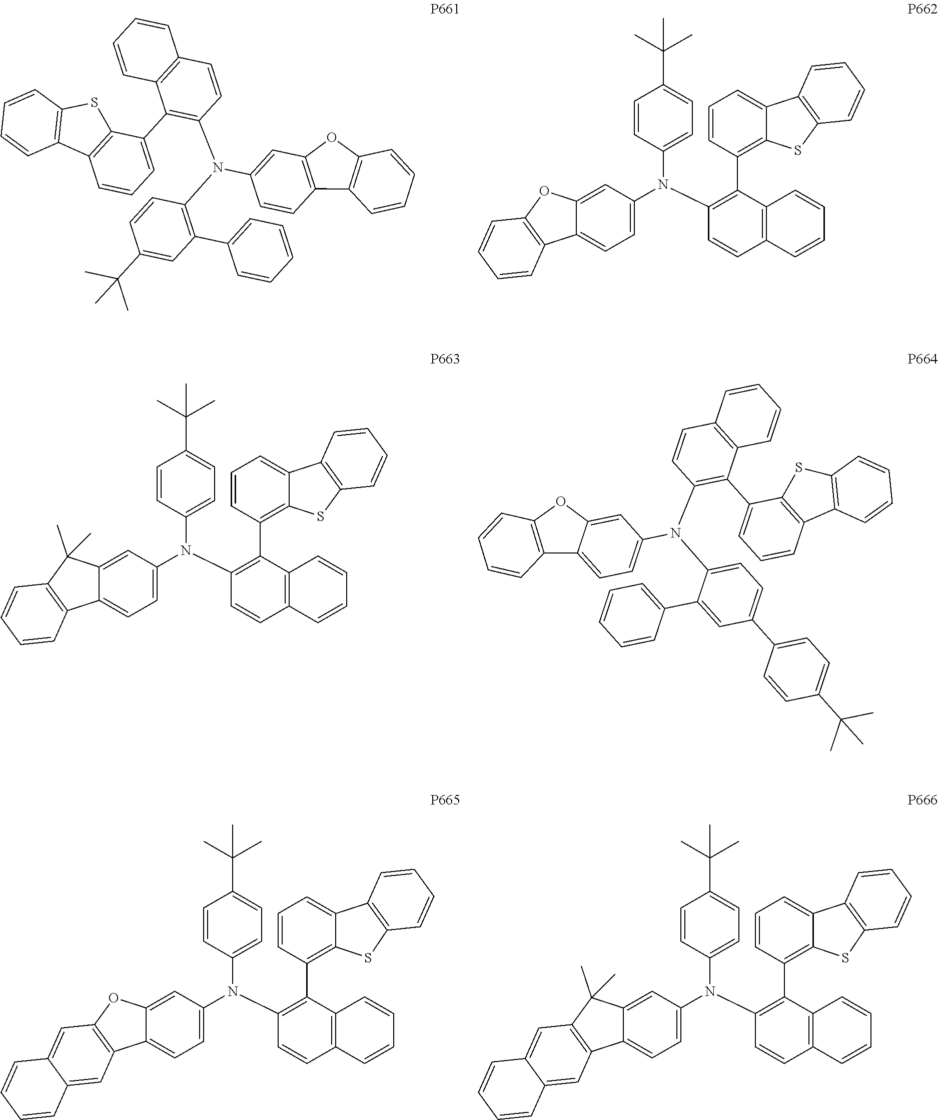

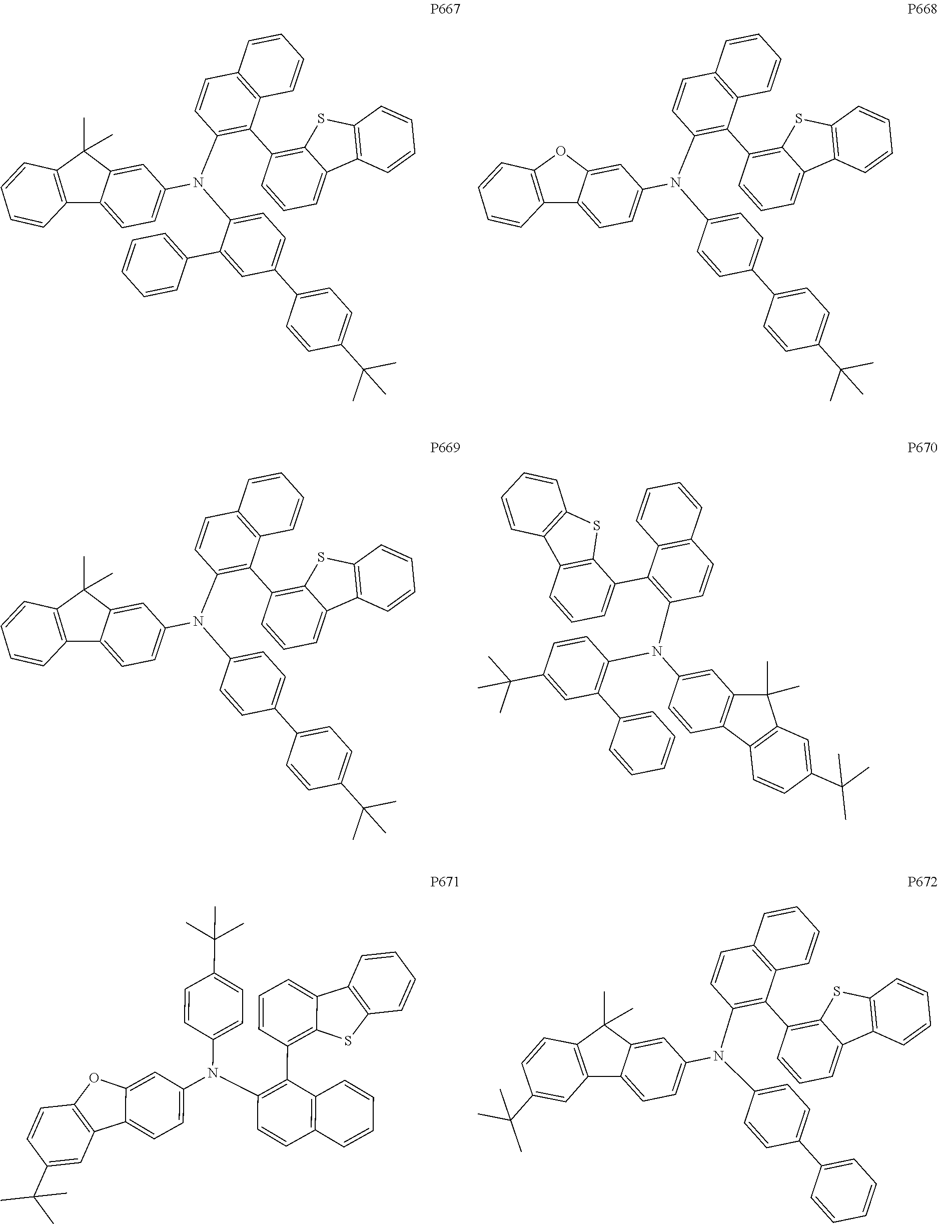

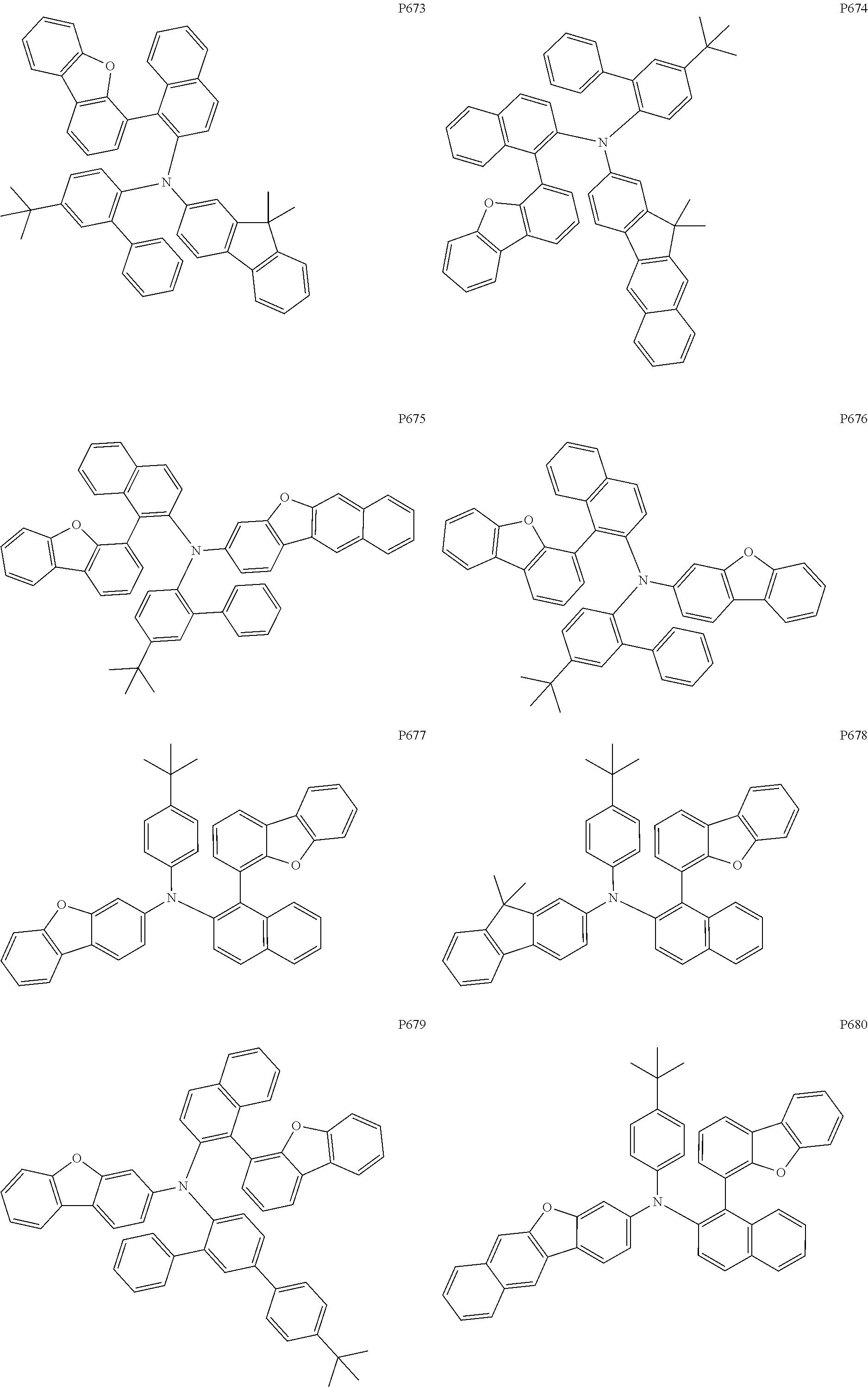

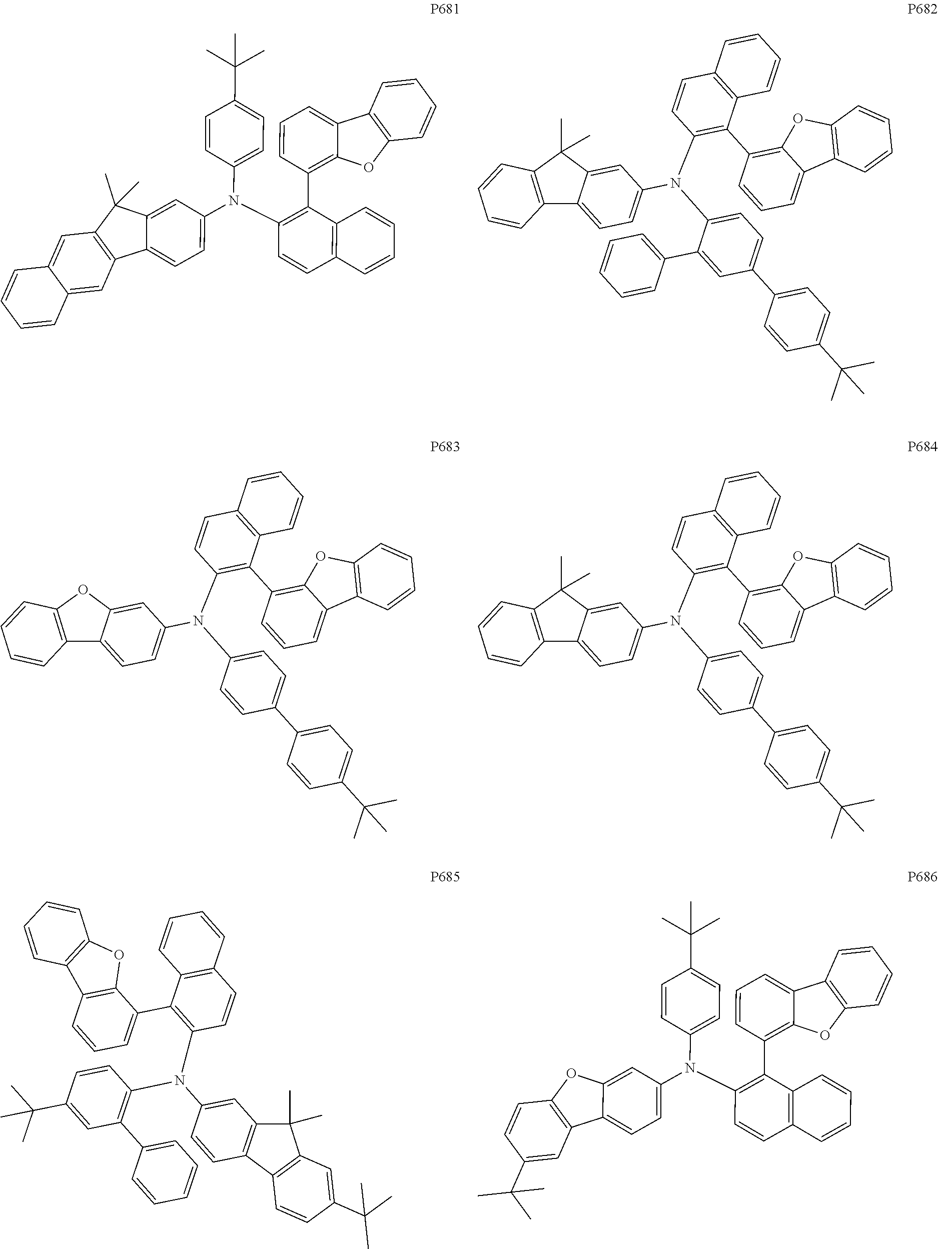

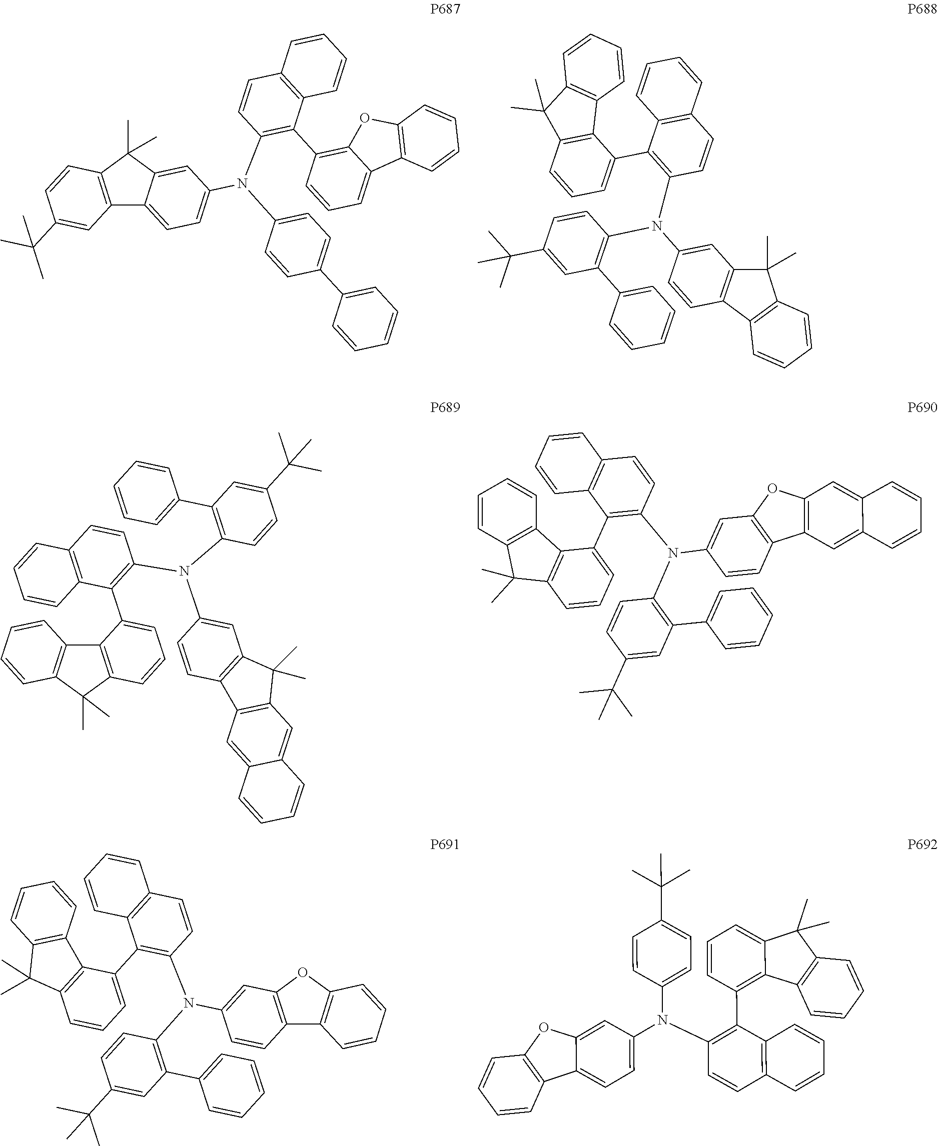

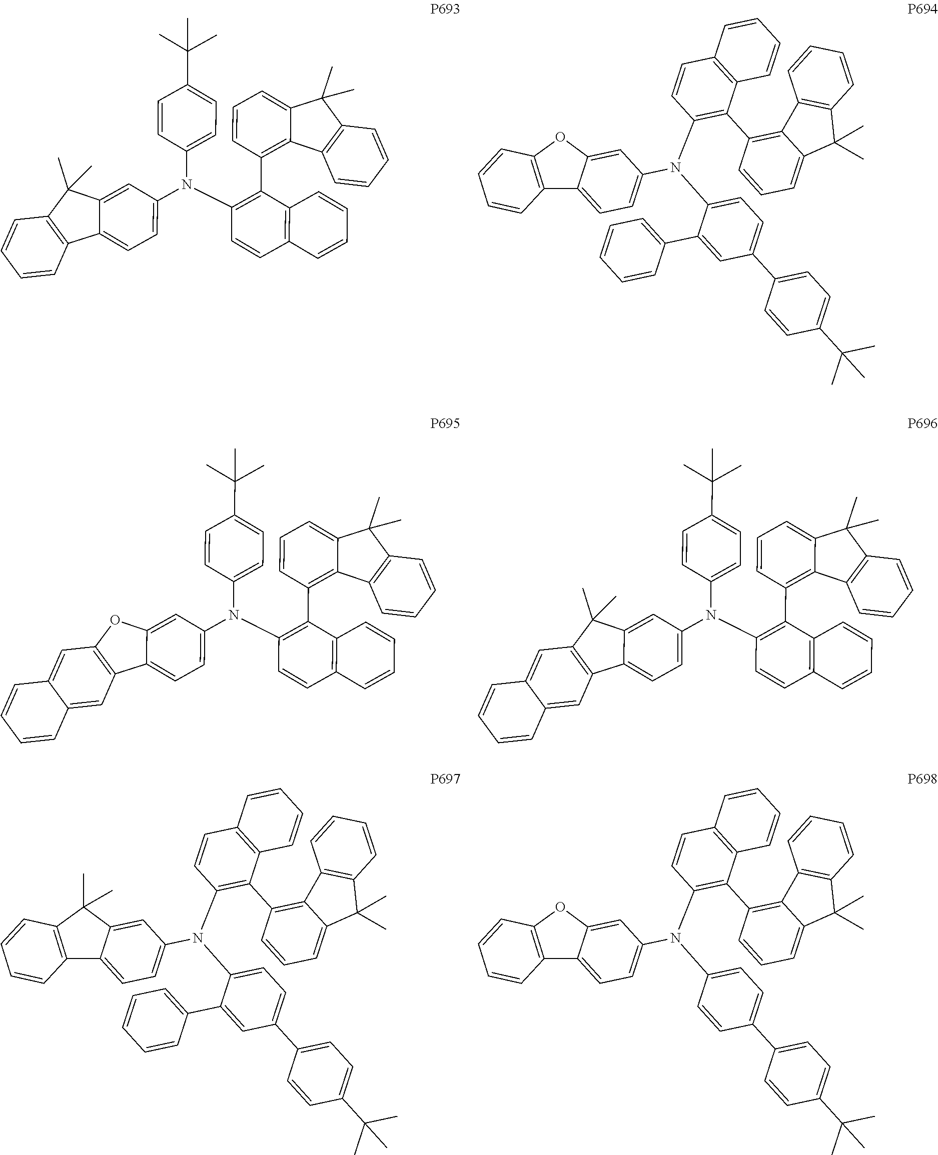

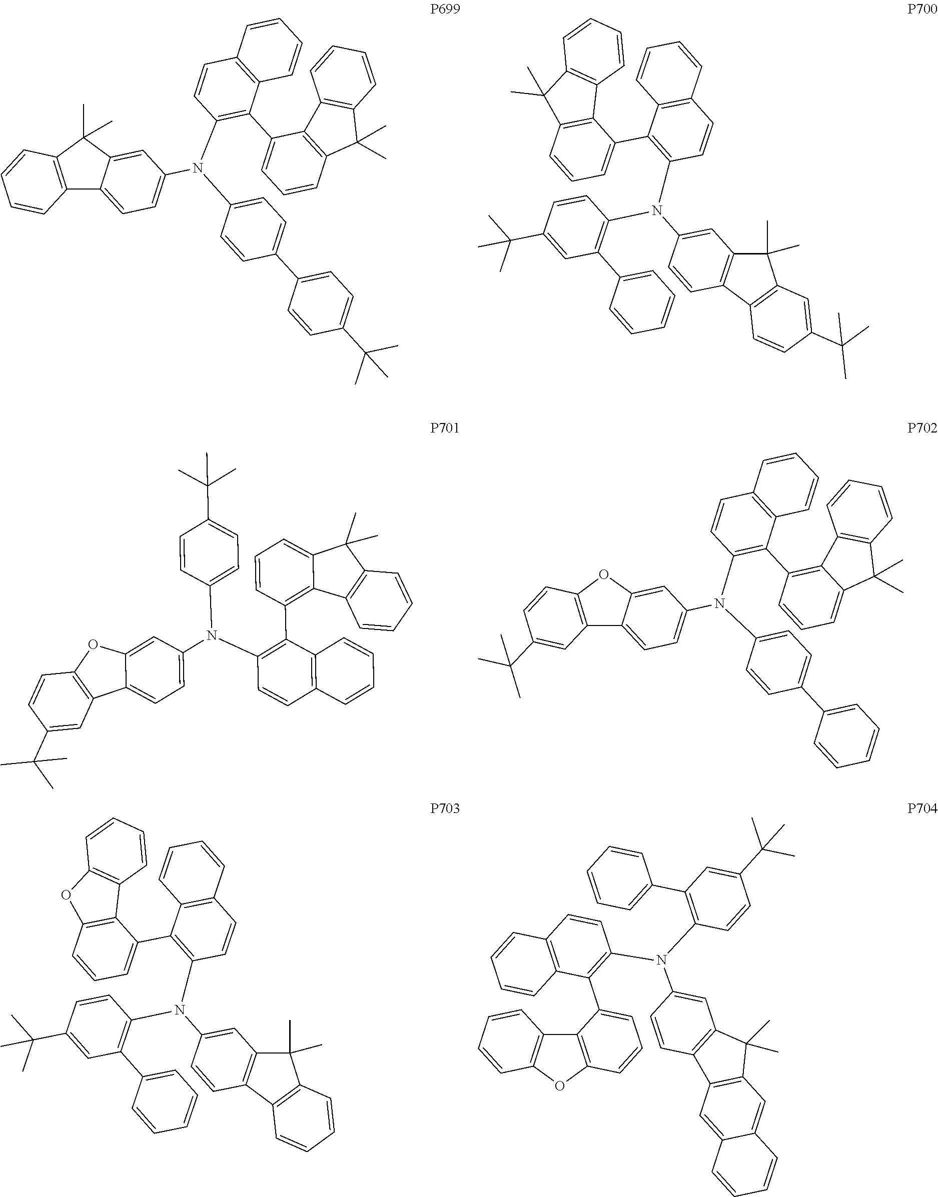

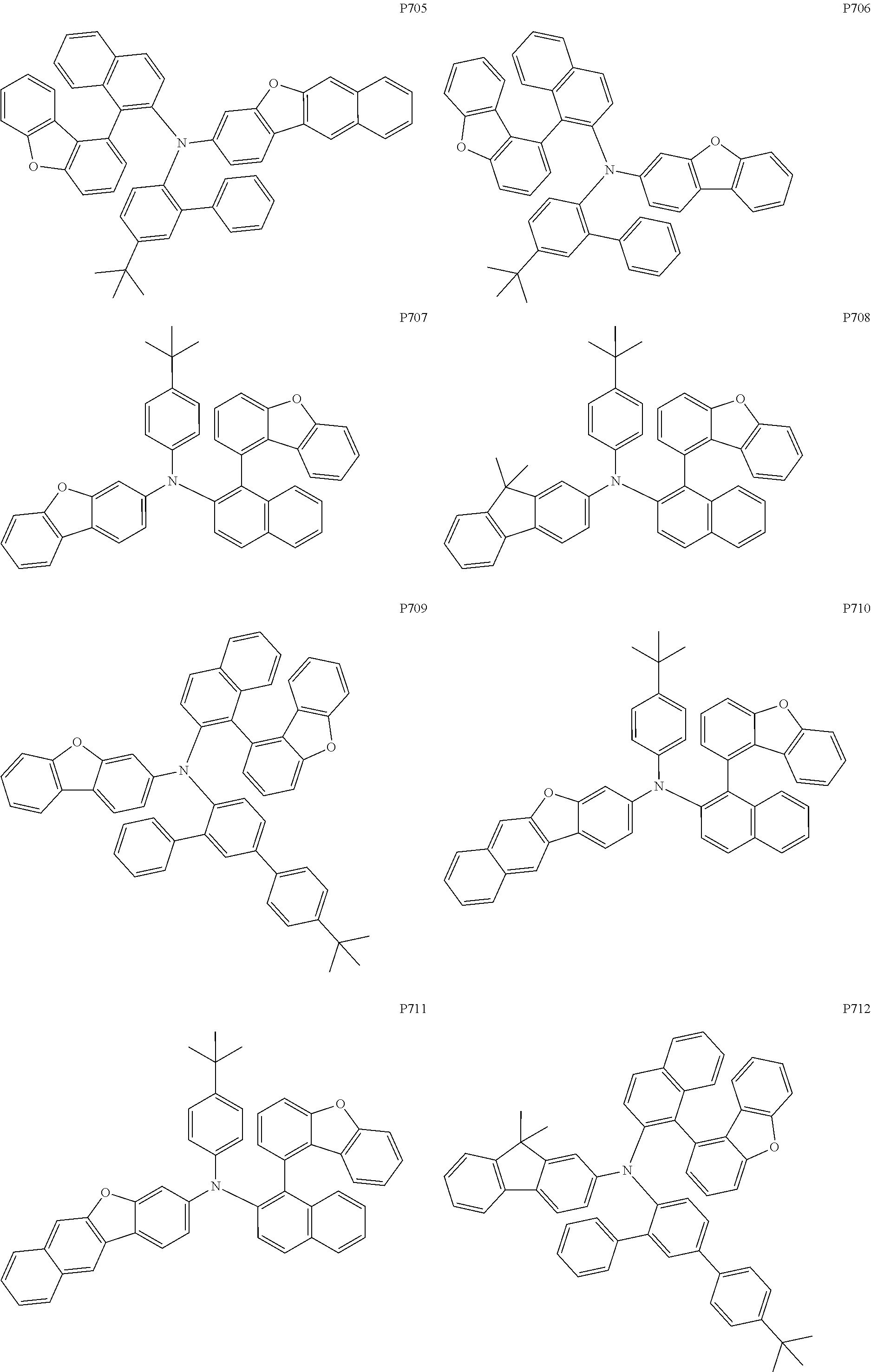

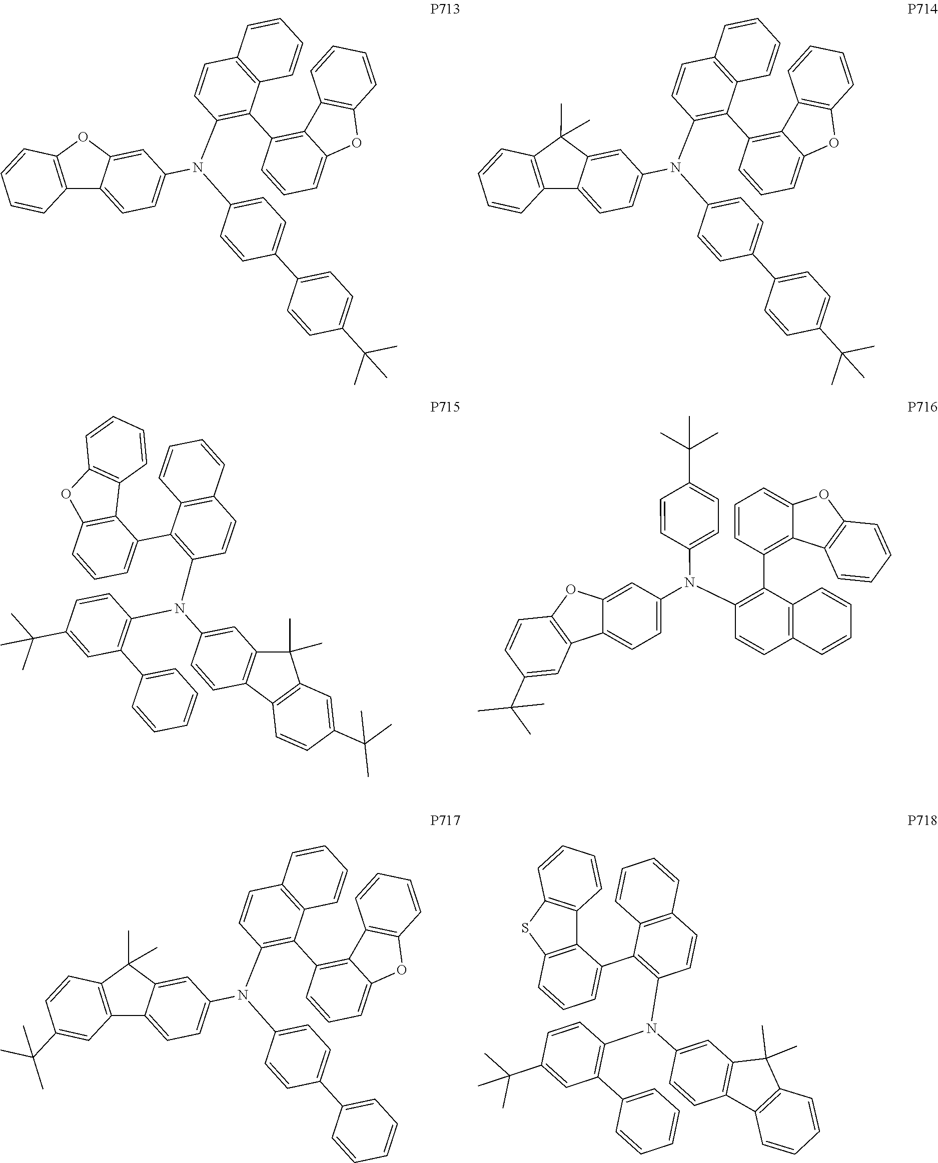

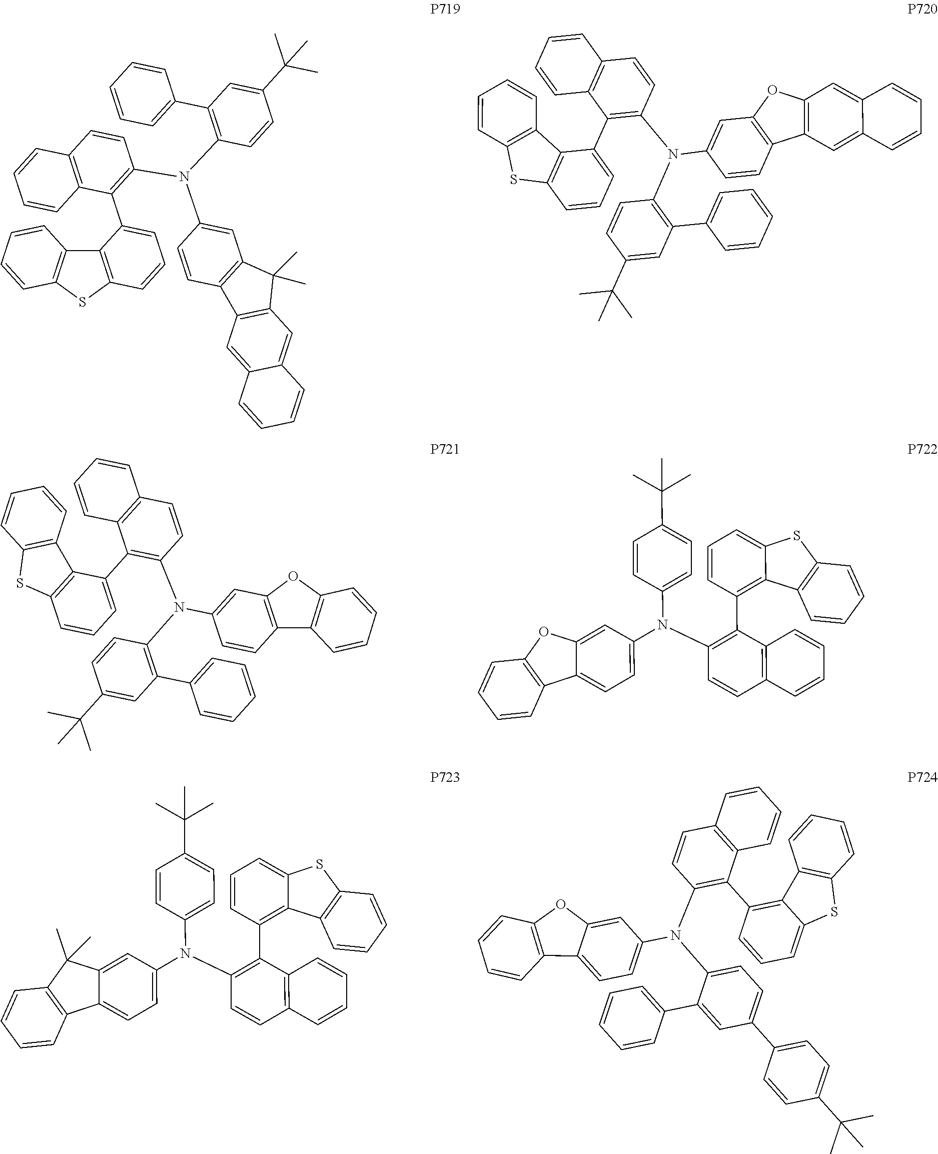

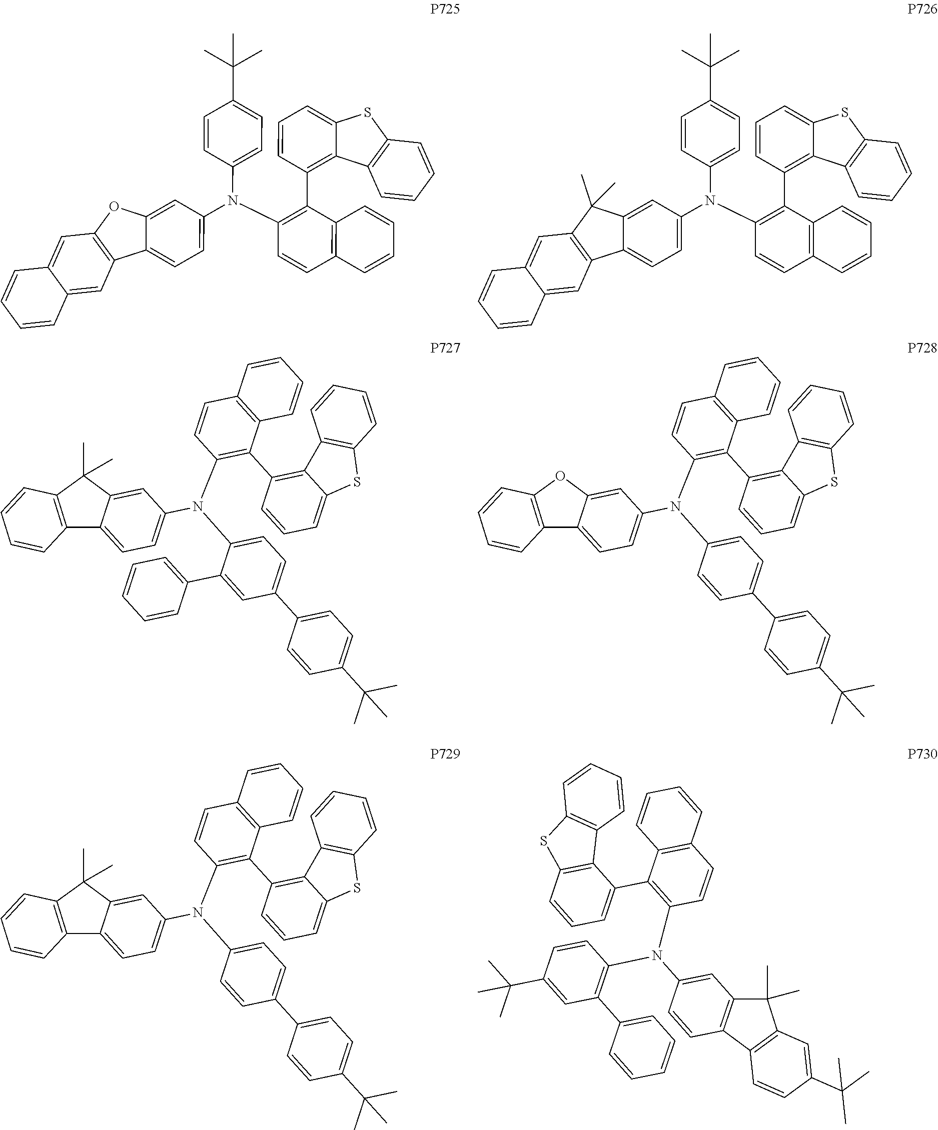

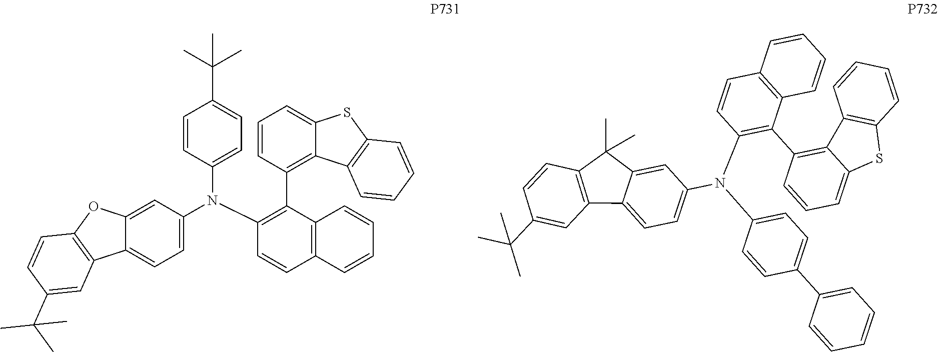 [SMILES: CC(C)(C)c1ccc(N(c2ccc3c(c2)oc2ccc(C(C)(C)C)cc23)c2ccc3ccccc3c2-c2cccc3sc4ccccc4c23)cc1 ; CC(C)(C)c1ccc2c(c1)-c1ccc(N(c3ccc(-c4ccccc4)cc3)c3ccc4ccccc4c3-c3cccc4sc5ccccc5c34)cc1C2(C)C]

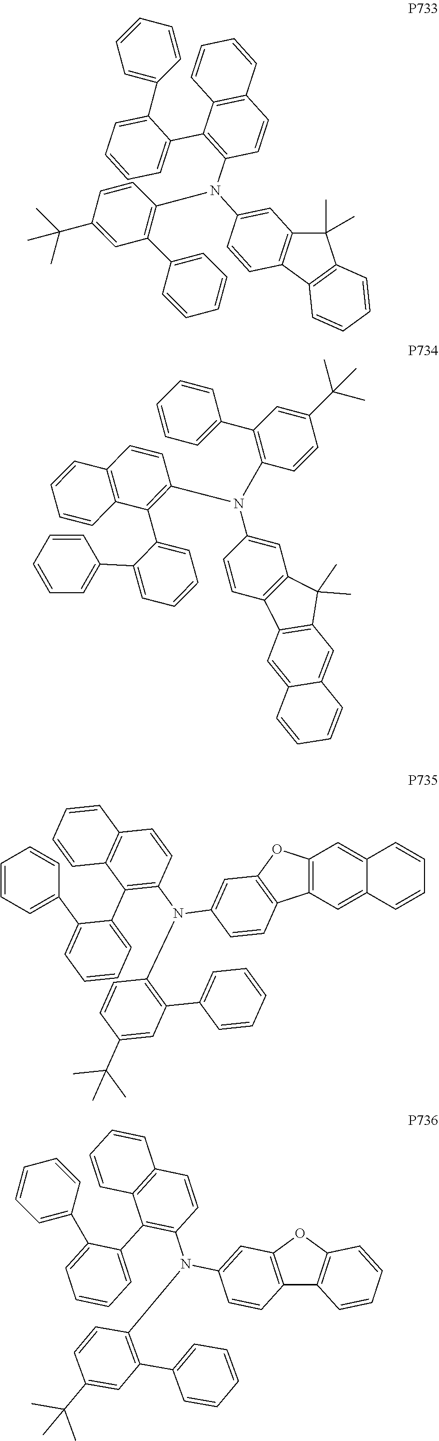

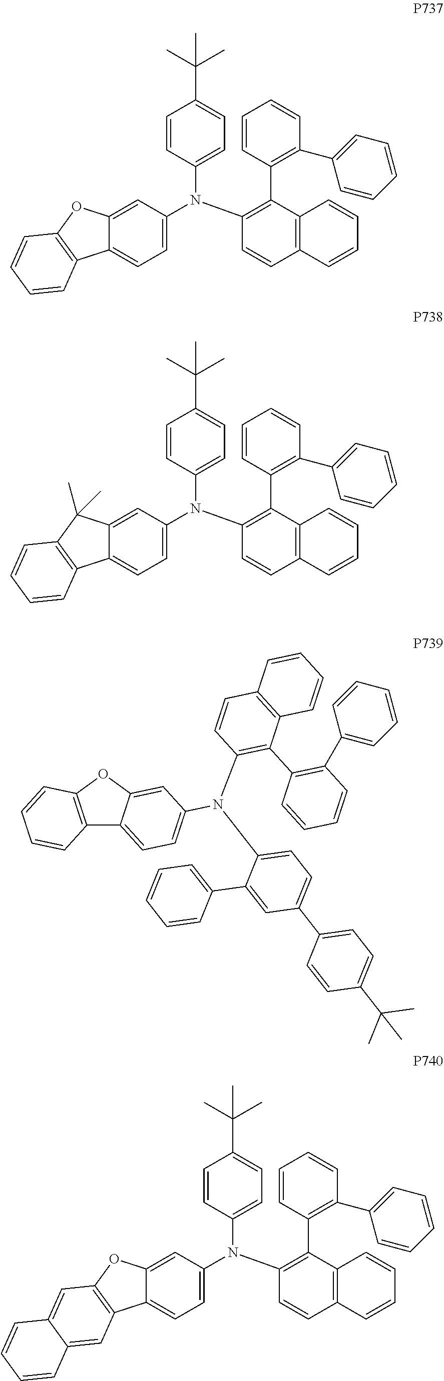 [SMILES: CC(C)(C)c1ccc(N(c2ccc3c(c2)oc2ccccc23)c2ccc3ccccc3c2-c2ccccc2-c2ccccc2)cc1 ; CC(C)(C)c1ccc(N(c2ccc3c(c2)C(C)(C)c2ccccc2-3)c2ccc3ccccc3c2-c2ccccc2-c2ccccc2)cc1 ; CC(C)(C)c1ccc(-c2ccc(N(c3ccc4c(c3)oc3ccccc34)c3ccc4ccccc4c3-c3ccccc3-c3ccccc3)c(-c3ccccc3)c2)cc1 ; CC(C)(C)c1ccc(N(c2ccc3c(c2)oc2cc4ccccc4cc23)c2ccc3ccccc3c2-c2ccccc2-c2ccccc2)cc1]

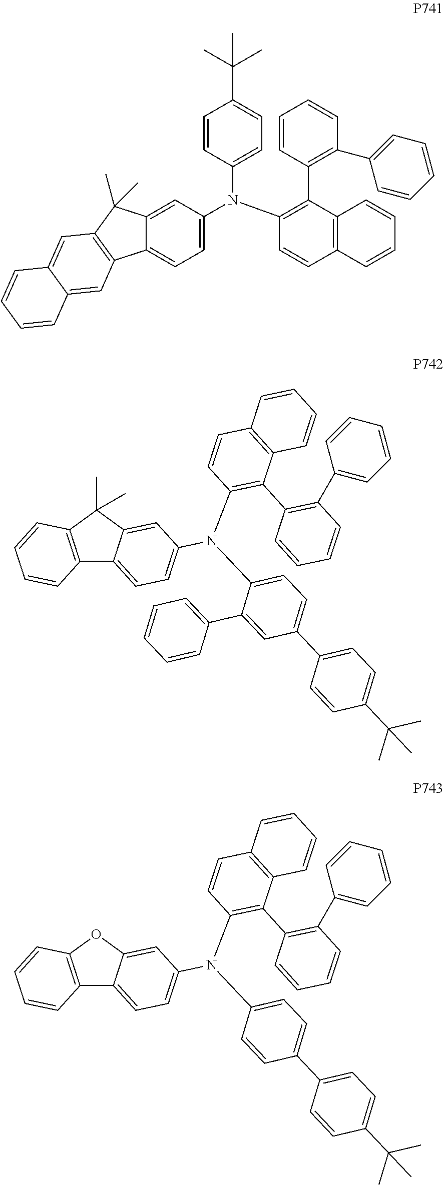 [SMILES: CC(C)(C)c1ccc(N(c2ccc3c(c2)C(C)(C)c2cc4ccccc4cc2-3)c2ccc3ccccc3c2-c2ccccc2-c2ccccc2)cc1 ; CC(C)(C)c1ccc(-c2ccc(N(c3ccc4c(c3)C(C)(C)c3ccccc3-4)c3ccc4ccccc4c3-c3ccccc3-c3ccccc3)c(-c3ccccc3)c2)cc1 ; CC(C)(C)c1ccc(-c2ccc(N(c3ccc4c(c3)oc3ccccc34)c3ccc4ccccc4c3-c3ccccc3-c3ccccc3)cc2)cc1]

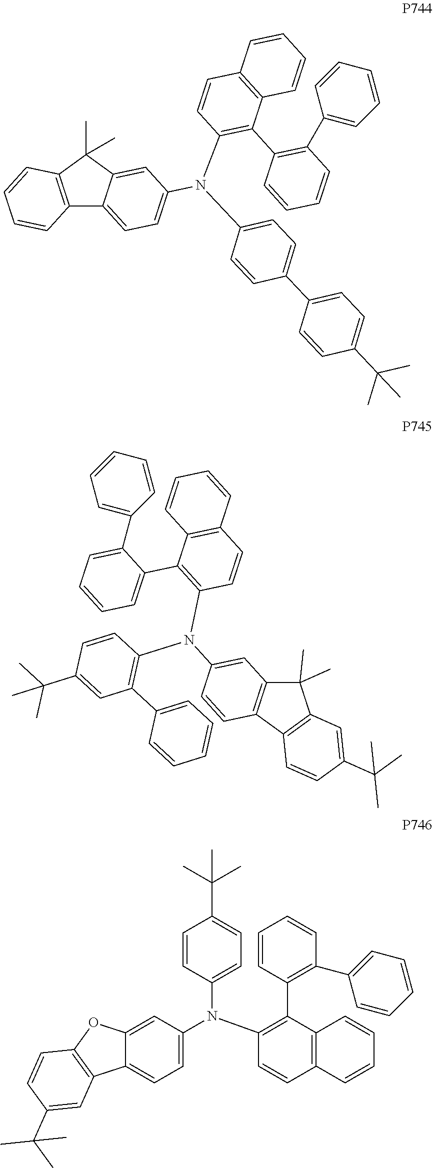

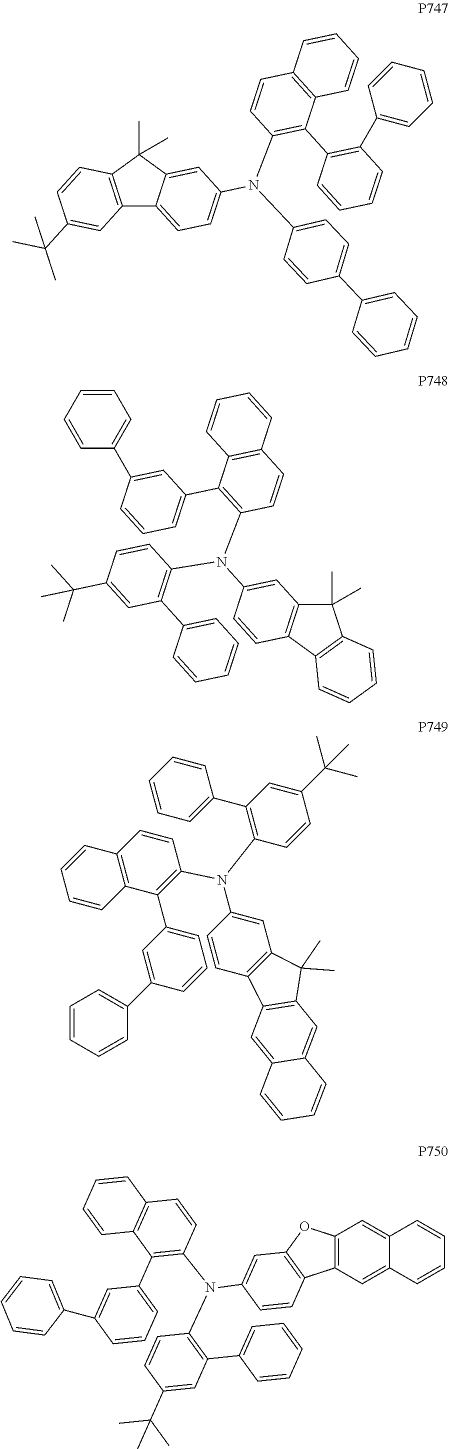

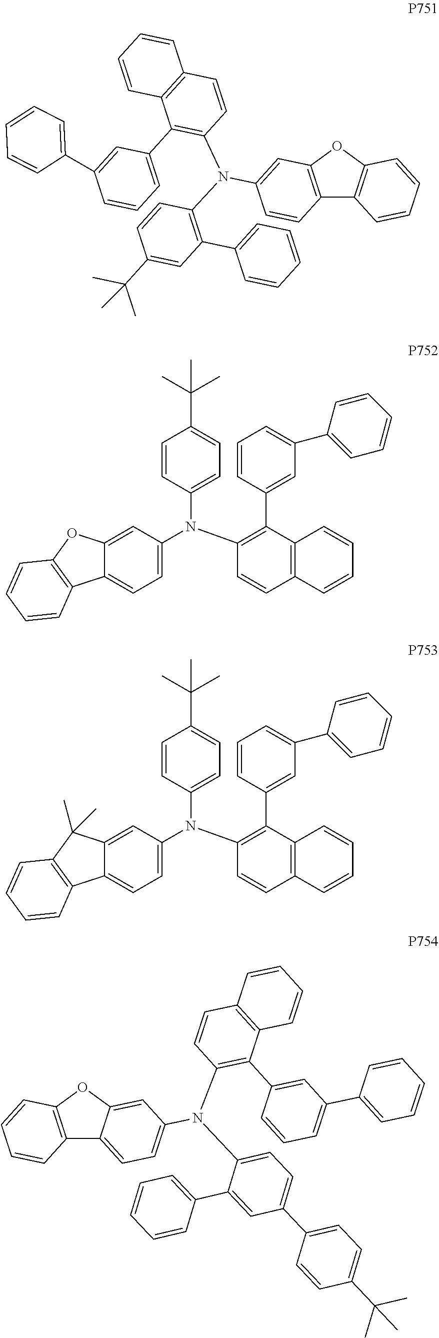 [SMILES: CC(C)(C)c1ccc(N(c2ccc3c(c2)oc2ccccc23)c2ccc3ccccc3c2-c2cccc(-c3ccccc3)c2)c(-c2ccccc2)c1 ; CC(C)(C)c1ccc(N(c2ccc3c(c2)oc2ccccc23)c2ccc3ccccc3c2-c2cccc(-c3ccccc3)c2)cc1 ; CC(C)(C)c1ccc(N(c2ccc3c(c2)C(C)(C)c2ccccc2-3)c2ccc3ccccc3c2-c2cccc(-c3ccccc3)c2)cc1 ; CC(C)(C)c1ccc(-c2ccc(N(c3ccc4c(c3)oc3ccccc34)c3ccc4ccccc4c3-c3cccc(-c4ccccc4)c3)c(-c3ccccc3)c2)cc1]

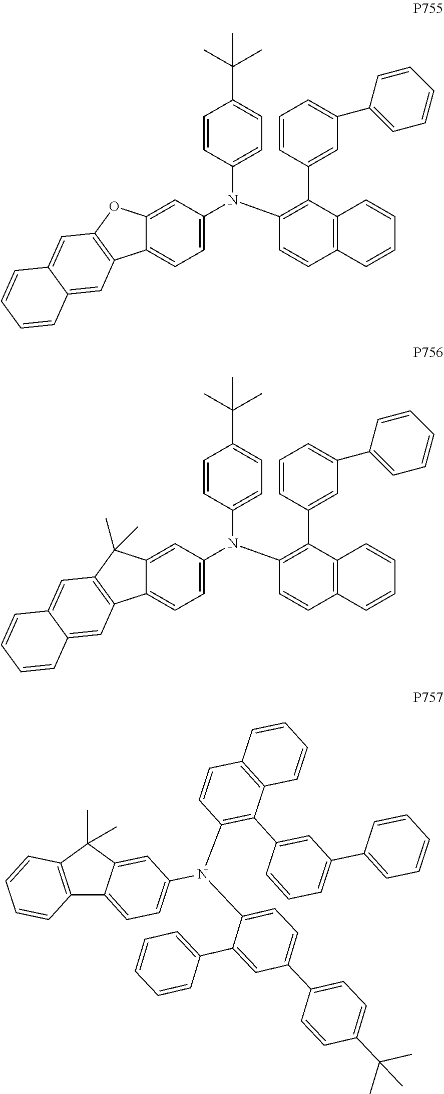 [SMILES: CC(C)(C)c1ccc(N(c2ccc3c(c2)oc2cc4ccccc4cc23)c2ccc3ccccc3c2-c2cccc(-c3ccccc3)c2)cc1 ; CC(C)(C)c1ccc(N(c2ccc3c(c2)C(C)(C)c2cc4ccccc4cc2-3)c2ccc3ccccc3c2-c2cccc(-c3ccccc3)c2)cc1 ; CC(C)(C)c1ccc(-c2ccc(N(c3ccc4c(c3)C(C)(C)c3ccccc3-4)c3ccc4ccccc4c3-c3cccc(-c4ccccc4)c3)c(-c3ccccc3)c2)cc1]

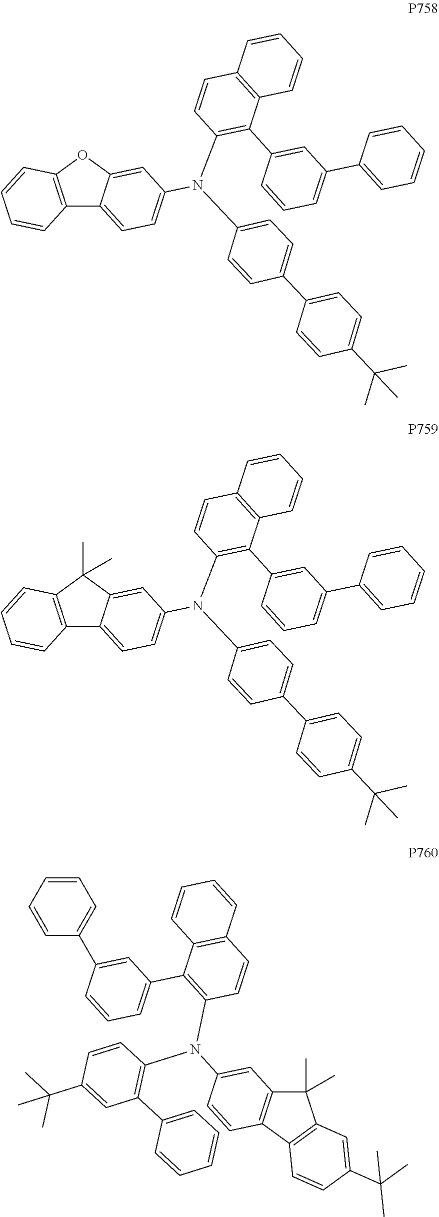 [SMILES: CC(C)(C)c1ccc(-c2ccc(N(c3ccc4c(c3)oc3ccccc34)c3ccc4ccccc4c3-c3cccc(-c4ccccc4)c3)cc2)cc1 ; CC(C)(C)c1ccc(-c2ccc(N(c3ccc4c(c3)C(C)(C)c3ccccc3-4)c3ccc4ccccc4c3-c3cccc(-c4ccccc4)c3)cc2)cc1 ; CC(C)(C)c1ccc(N(c2ccc3c(c2)C(C)(C)c2cc(C(C)(C)C)ccc2-3)c2ccc3ccccc3c2-c2cccc(-c3ccccc3)c2)c(-c2ccccc2)c1]

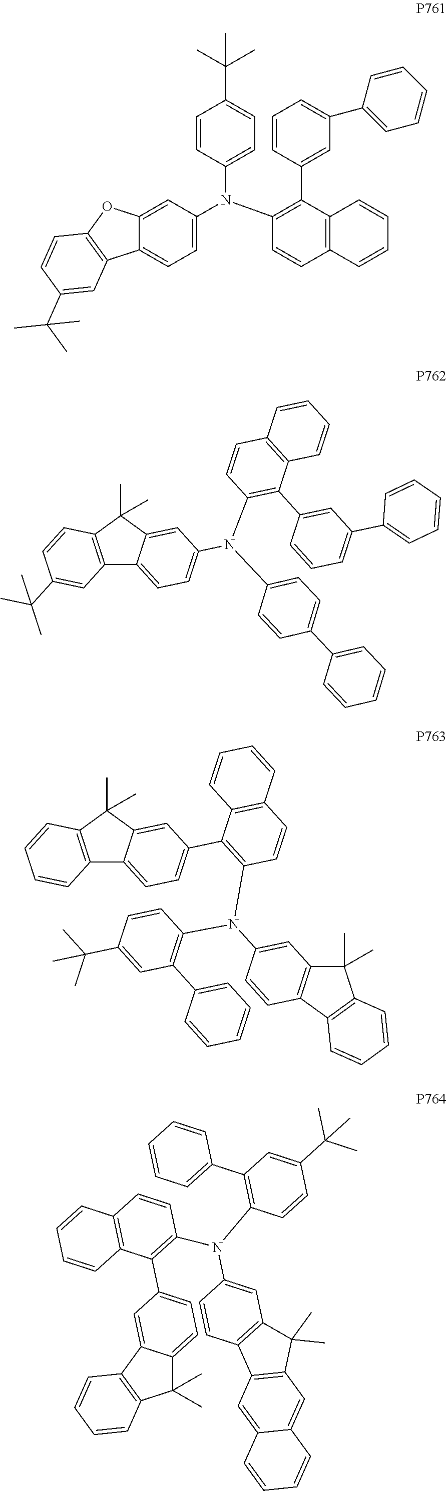 [SMILES: CC(C)(C)c1ccc(N(c2ccc3c(c2)oc2ccc(C(C)(C)C)cc23)c2ccc3ccccc3c2-c2cccc(-c3ccccc3)c2)cc1 ; CC(C)(C)c1ccc2c(c1)-c1ccc(N(c3ccc(-c4ccccc4)cc3)c3ccc4ccccc4c3-c3cccc(-c4ccccc4)c3)cc1C2(C)C ; CC(C)(C)c1ccc(N(c2ccc3c(c2)C(C)(C)c2ccccc2-3)c2ccc3ccccc3c2-c2ccc3c(c2)C(C)(C)c2ccccc2-3)c(-c2ccccc2)c1 ; CC(C)(C)c1ccc(N(c2ccc3c(c2)C(C)(C)c2cc4ccccc4cc2-3)c2ccc3ccccc3c2-c2ccc3c(c2)-c2ccccc2C3(C)C)c(-c2ccccc2)c1]

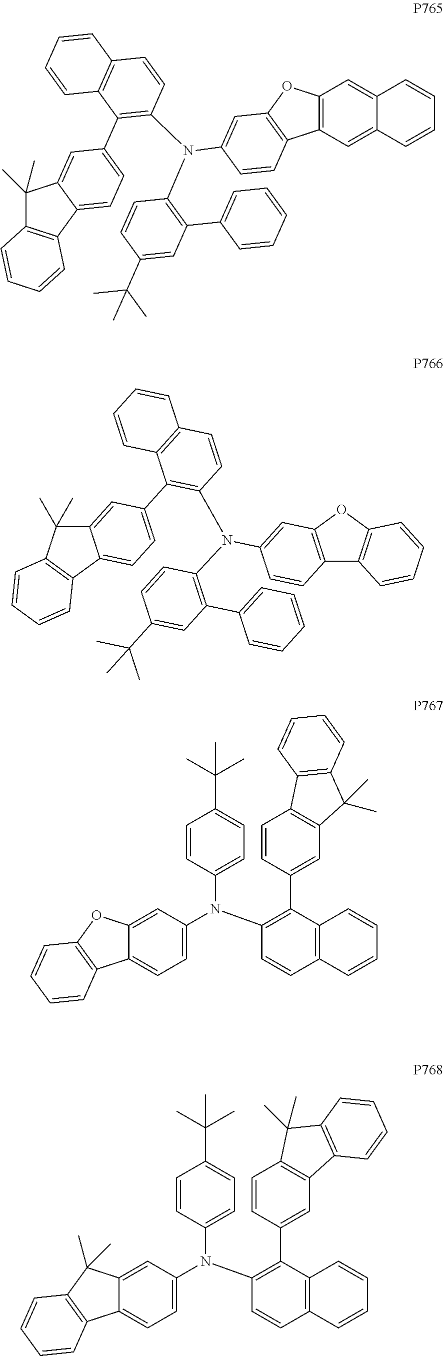 [SMILES: CC(C)(C)c1ccc(N(c2ccc3c(c2)oc2cc4ccccc4cc23)c2ccc3ccccc3c2-c2ccc3c(c2)C(C)(C)c2ccccc2-3)c(-c2ccccc2)c1 ; CC(C)(C)c1ccc(N(c2ccc3c(c2)oc2ccccc23)c2ccc3ccccc3c2-c2ccc3c(c2)C(C)(C)c2ccccc2-3)c(-c2ccccc2)c1 ; CC(C)(C)c1ccc(N(c2ccc3c(c2)oc2ccccc23)c2ccc3ccccc3c2-c2ccc3c(c2)C(C)(C)c2ccccc2-3)cc1 ; CC(C)(C)c1ccc(N(c2ccc3c(c2)C(C)(C)c2ccccc2-3)c2ccc3ccccc3c2-c2ccc3c(c2)-c2ccccc2C3(C)C)cc1]

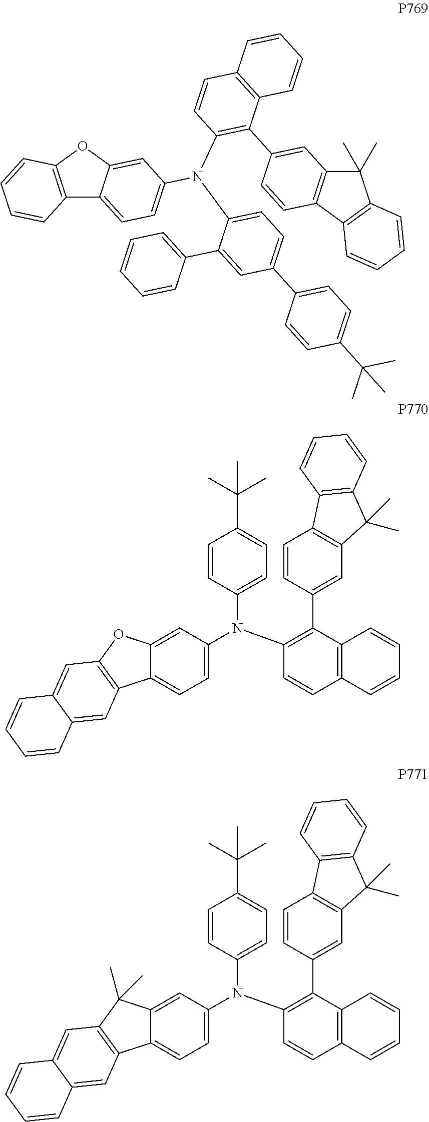

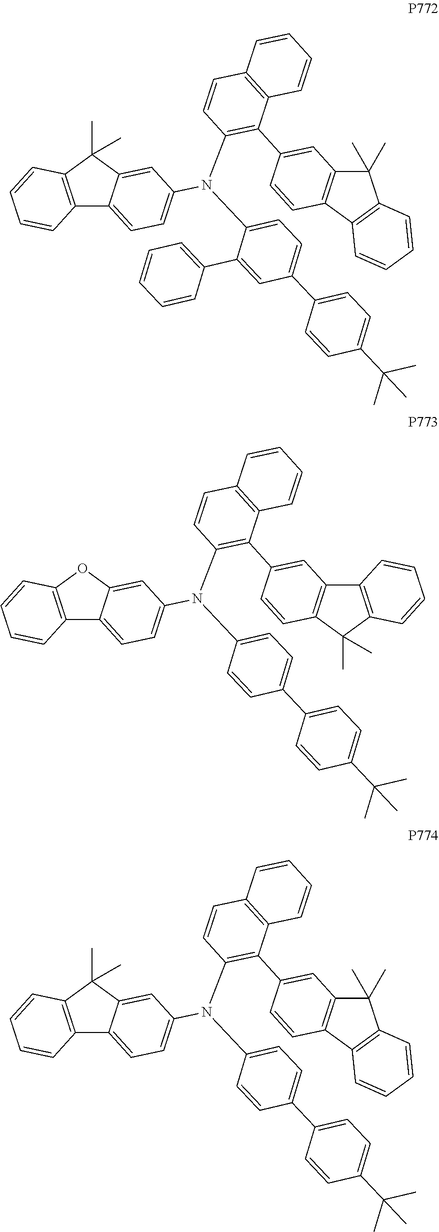 [SMILES: CC(C)(C)c1ccc(-c2ccc(N(c3ccc4c(c3)C(C)(C)c3ccccc3-4)c3ccc4ccccc4c3-c3ccc4c(c3)C(C)(C)c3ccccc3-4)c(-c3ccccc3)c2)cc1 ; CC(C)(C)c1ccc(-c2ccc(N(c3ccc4c(c3)oc3ccccc34)c3ccc4ccccc4c3-c3ccc4c(c3)-c3ccccc3C4(C)C)cc2)cc1 ; CC(C)(C)c1ccc(-c2ccc(N(c3ccc4c(c3)C(C)(C)c3ccccc3-4)c3ccc4ccccc4c3-c3ccc4c(c3)C(C)(C)c3ccccc3-4)cc2)cc1]

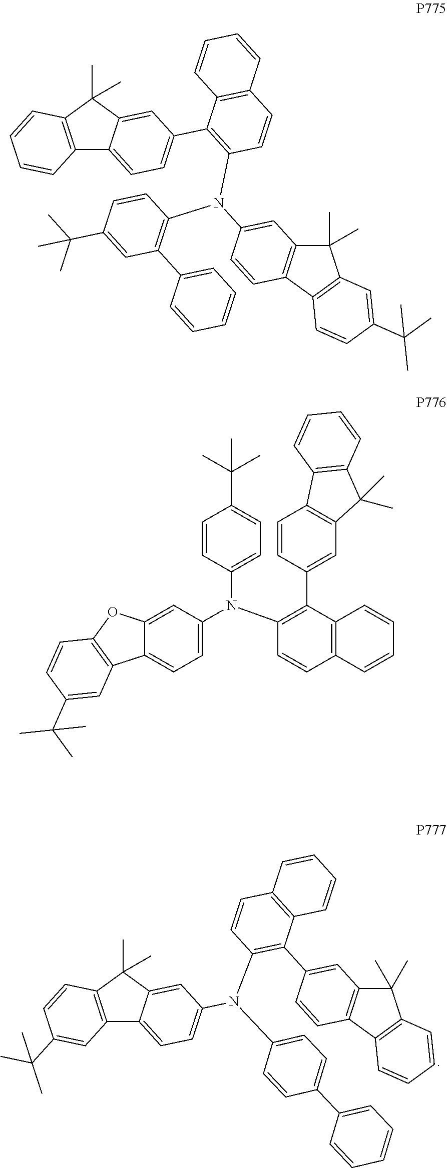

12. An organic electroluminescent device, wherein, the organic electroluminescent device comprises a first electrode, a second electrode and at least one organic layer inserted between the first electrode and the second electrode, wherein the organic layer contains at least one compound according to claim 1.

13. The organic electroluminescent device according to claim 12, wherein, the organic layer comprises an electron blocking layer, wherein the electron blocking layer comprises at least one compound according to claim 1.

14. The organic electroluminescent device according to claim 12, wherein, the first electrode is an anode layer, the second electrode is a cathode layer, the organic layer comprises a light emitting layer, and the light emitting layer contains a host material and a doping material; the host material comprises a first host material and a second host material, and the first host material is a compound according to claim 1.

15. The organic electroluminescent device according to claim 14, wherein, In Formula I, the Ar¹ is selected from the group consisting of any of the following substituted or unsubstituted groups: phenyl, biphenyl, terphenyl, naphthyl, phenanthryl, anthracyl, dibenzofuranyl, dibenzothiophenyl, carbazolyl, 9,9dimethylfluorenyl, 9,9diphenylfluorenyl, spirofluorenyl, triphenylene, fluoranthenyl, benzo9,9dimethylfluorenyl, and benzospirofluorenyl;
and/or, In Formula I, the Ar² is selected from the group consisting of any of the following substituted or unsubstituted groups: phenyl, biphenyl, terphenyl, naphthyl, phenanthryl, anthracyl, dibenzofuranyl, dibenzothiophenyl, carbazolyl, 9,9dimethylfluorenyl, 9,9diphenylfluorenyl, spirofluorenyl, triphenylene, fluoranthenyl, benzo9,9dimethylfluorenyl, benzospirofluorenyl, preferably a substituted or unsubstituted naphthyl;
preferably, In Formula I, the R¹, R² and R³ are independently selected from the group consisting of one of methyl, ethyl or phenyl, preferably all are methyl;
preferably, the first host material is selected from the group consisting of any one or a combination of at least two of the compounds according to claim 6;
preferably, the mass ratio of the first host material to the second host material is 0.01:1-1.5:1, preferably 0.1:1-1:1.

16. (canceled)

17. (canceled)

18. (canceled)

19. The organic electroluminescent device according to claim 14, wherein, the HOMO energy level of the second host material is −5.3 eV to −5.7 eV;
and/or, the LUMO energy level of the second host material is −2.3 eV to −2.6 eV;
preferably, the HOMO energy level of the second host material is −5.3 eV to −5.7 eV, and the LUMO energy level is −2.3 eV to −2.6 eV;
preferably, the second host material is selected from the group consisting of any one or a combination of at least two of the following compound PH-1 to compound PH-85:

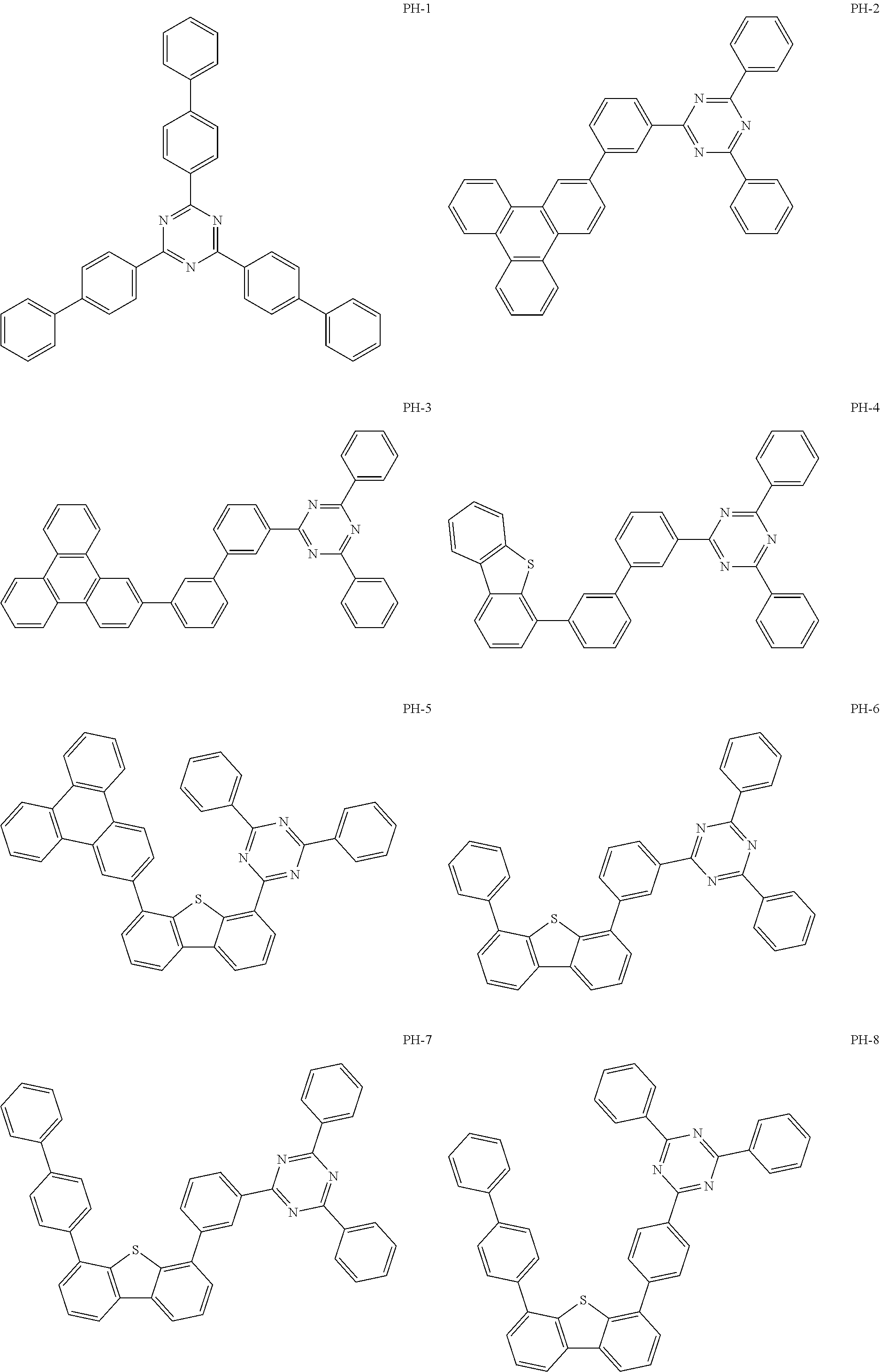

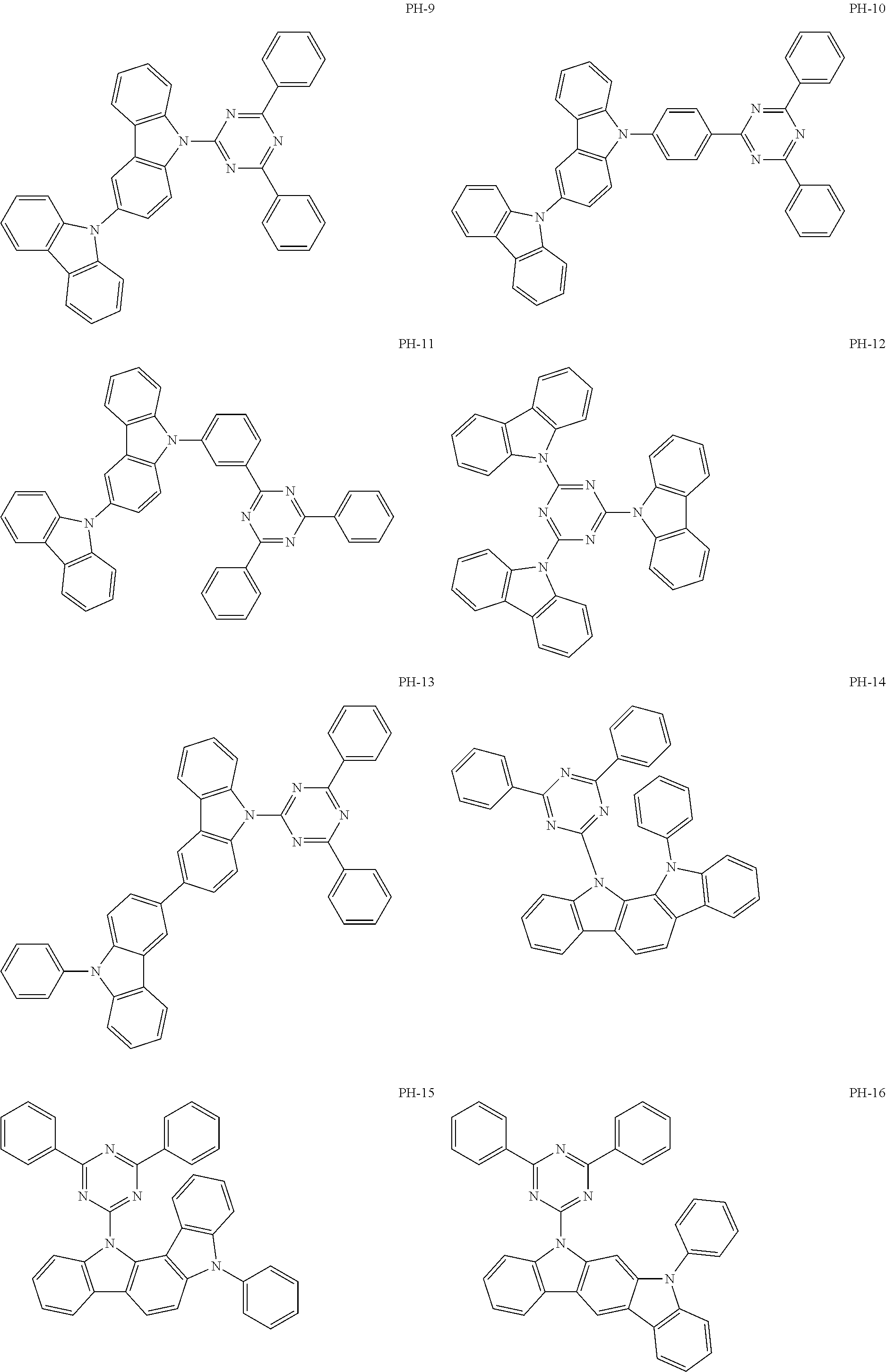

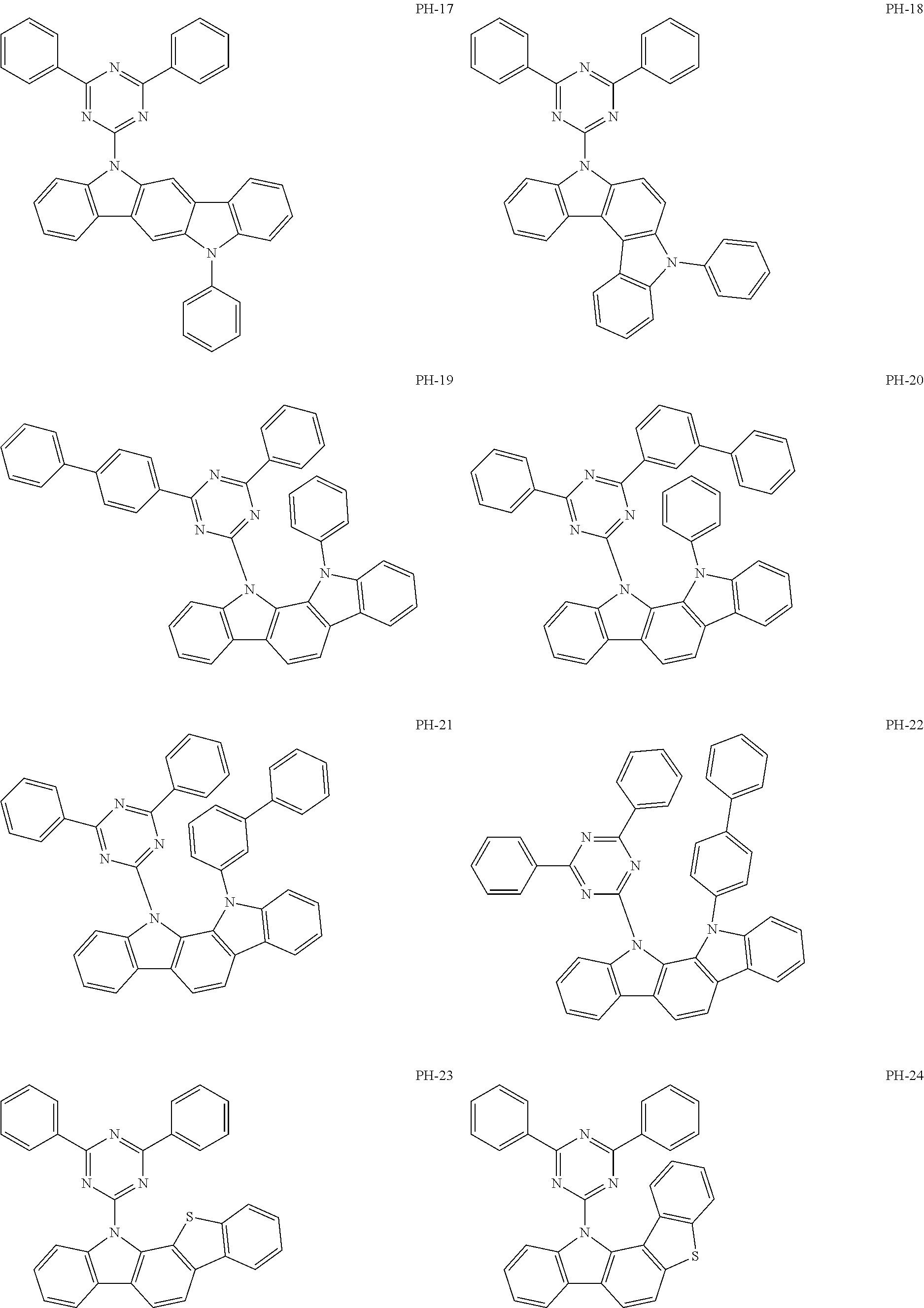

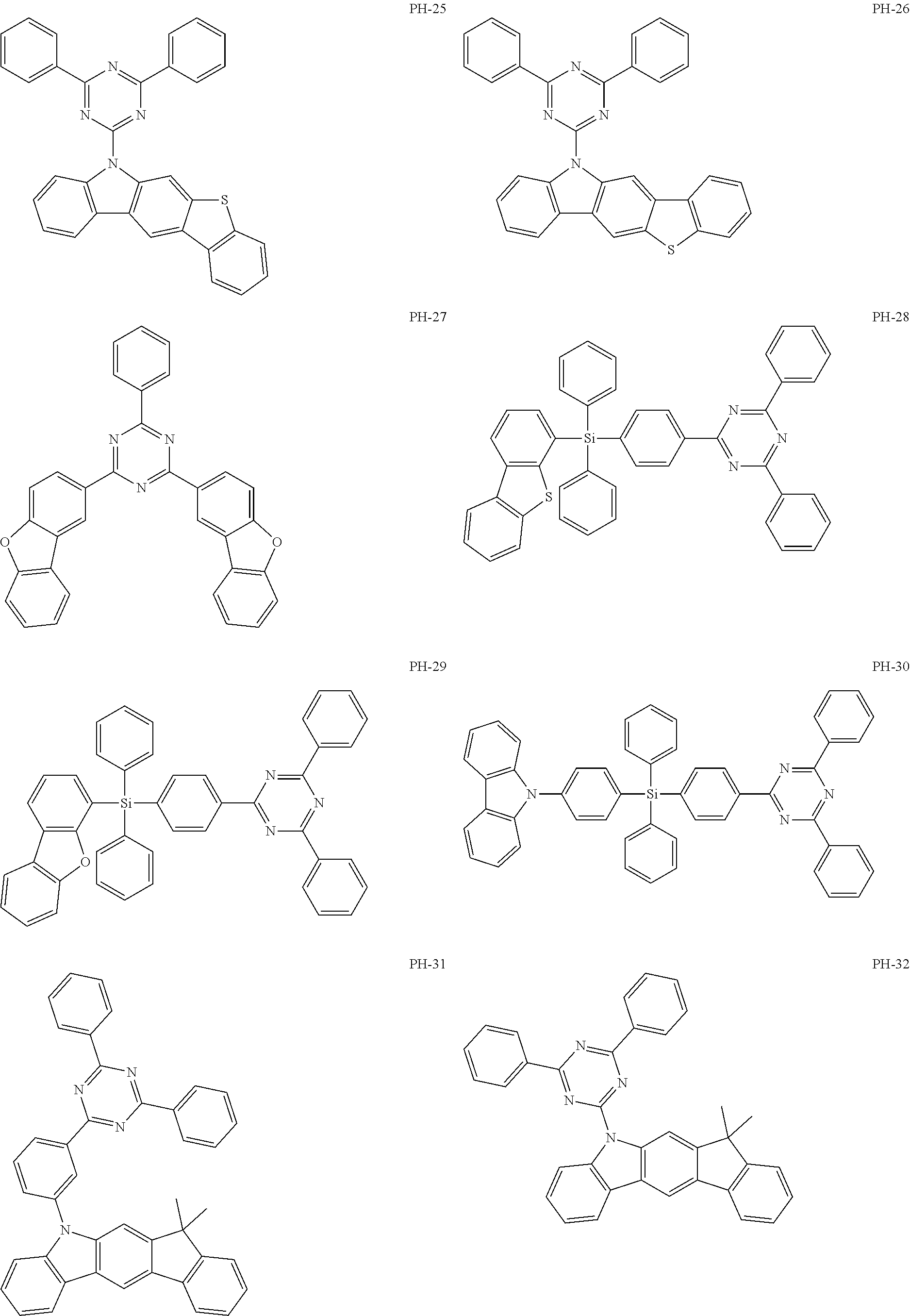

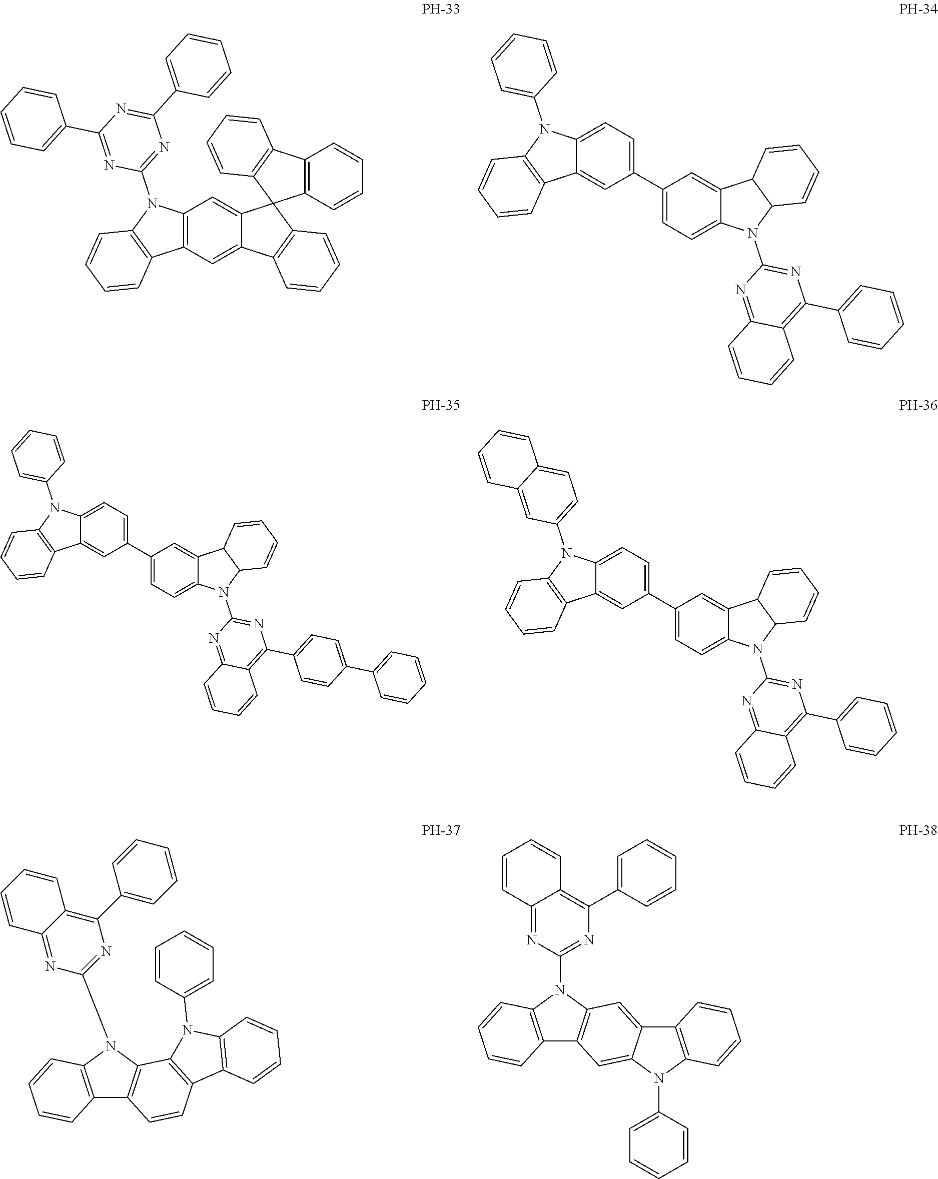 [SMILES: c1ccc(-c2nc(-c3ccccc3)nc(-n3c4ccccc4c4cc5c(cc43)C3(c4ccccc4-c4ccccc43)c3ccccc3-5)n2)cc1 ; C1=CC2c3cc(-c4ccc5c(c4)c4ccccc4n5-c4ccccc4)ccc3N(c3nc(-c4ccccc4)c4ccccc4n3)C2C=C1 ; C1=CC2c3cc(-c4ccc5c(c4)c4ccccc4n5-c4ccccc4)ccc3N(c3nc(-c4ccc(-c5ccccc5)cc4)c4ccccc4n3)C2C=C1 ; C1=CC2c3cc(-c4ccc5c(c4)c4ccccc4n5-c4ccc5ccccc5c4)ccc3N(c3nc(-c4ccccc4)c4ccccc4n3)C2C=C1 ; c1ccc(-c2nc(-n3c4ccccc4c4ccc5c6ccccc6n(-c6ccccc6)c5c43)nc3ccccc23)cc1 ; c1ccc(-c2nc(-n3c4ccccc4c4cc5c(cc43)c3ccccc3n5-c3ccccc3)nc3ccccc23)cc1]

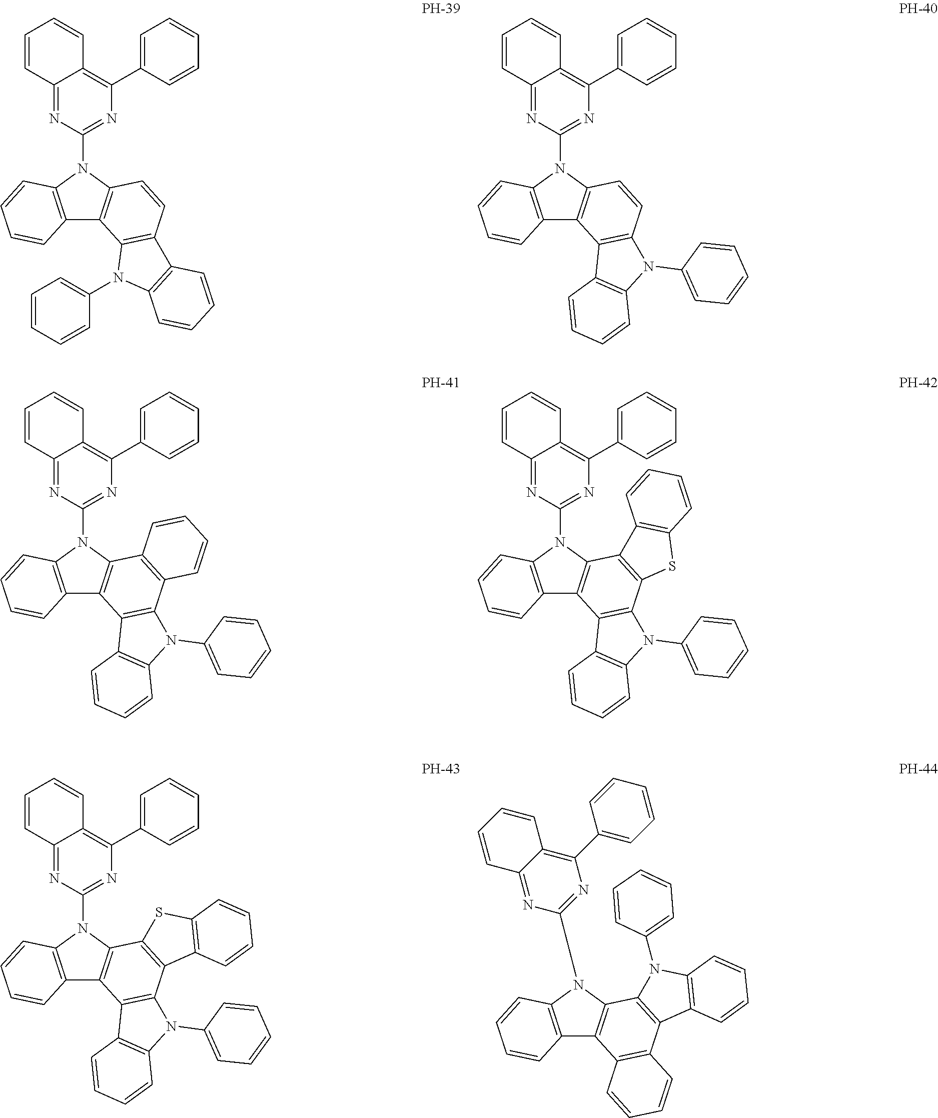 [SMILES: c1ccc(-c2nc(-n3c4ccccc4c4c3ccc3c5ccccc5n(-c5ccccc5)c34)nc3ccccc23)cc1 ; c1ccc(-c2nc(-n3c4ccccc4c4c5c6ccccc6n(-c6ccccc6)c5ccc43)nc3ccccc23)cc1 ; c1ccc(-c2nc(-n3c4ccccc4c4c5c6ccccc6n(-c6ccccc6)c5c5ccccc5c43)nc3ccccc23)cc1 ; c1ccc(-c2nc(-n3c4ccccc4c4c5c6ccccc6n(-c6ccccc6)c5c5sc6ccccc6c5c43)nc3ccccc23)cc1 ; c1ccc(-c2nc(-n3c4ccccc4c4c5c6ccccc6n(-c6ccccc6)c5c5c6ccccc6sc5c43)nc3ccccc23)cc1 ; c1ccc(-c2nc(-n3c4ccccc4c4c5ccccc5c5c6ccccc6n(-c6ccccc6)c5c43)nc3ccccc23)cc1]

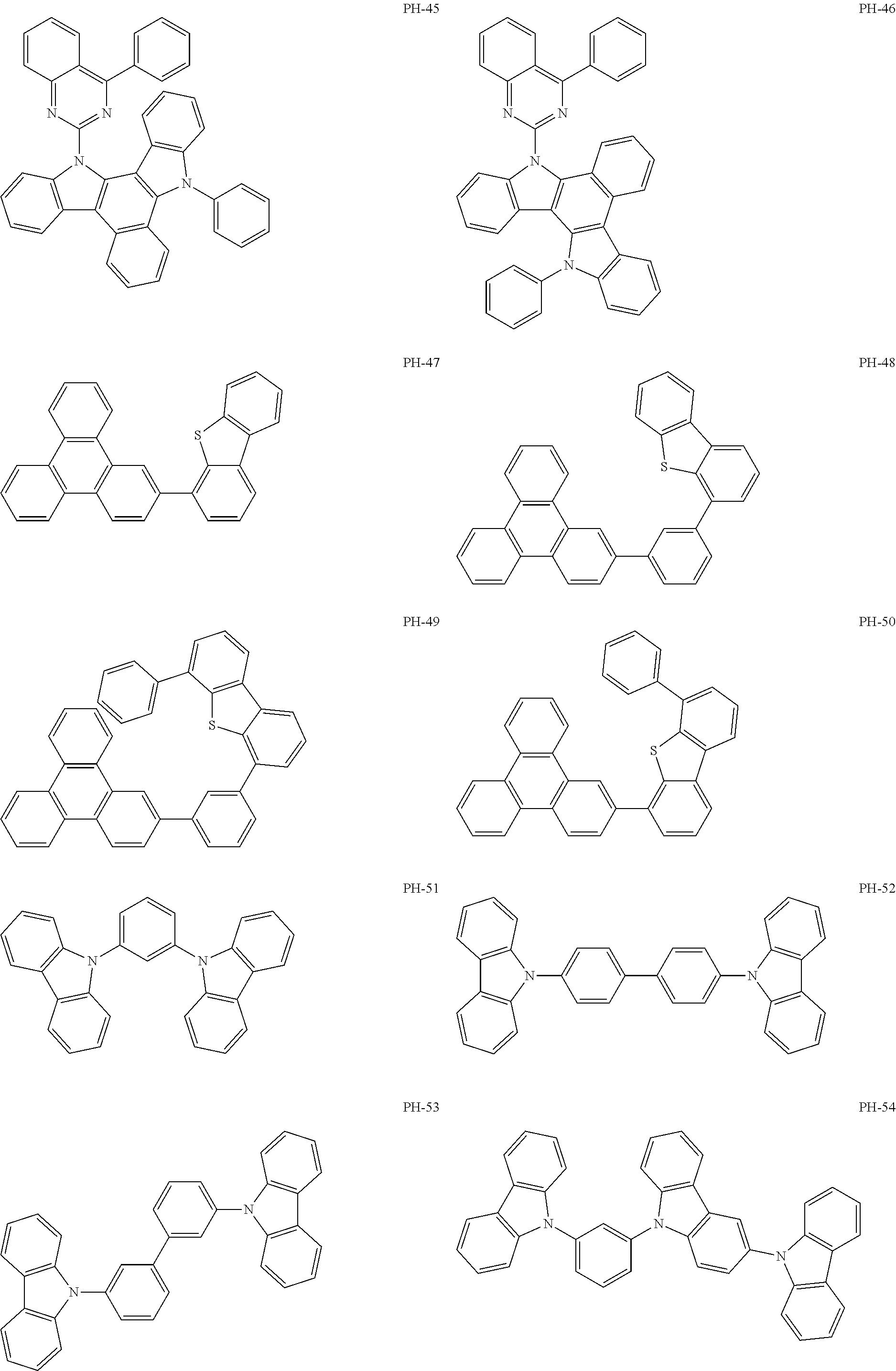

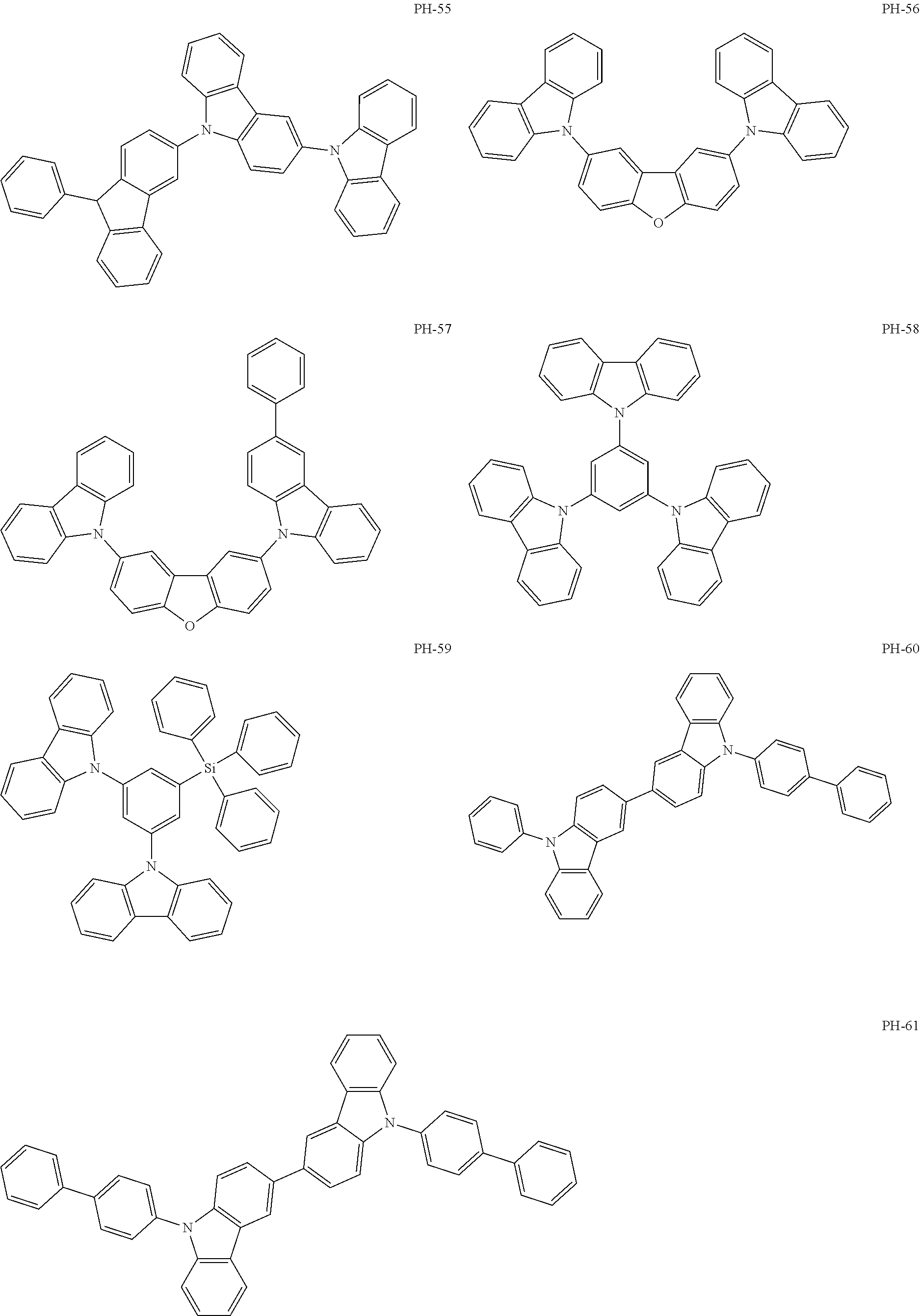

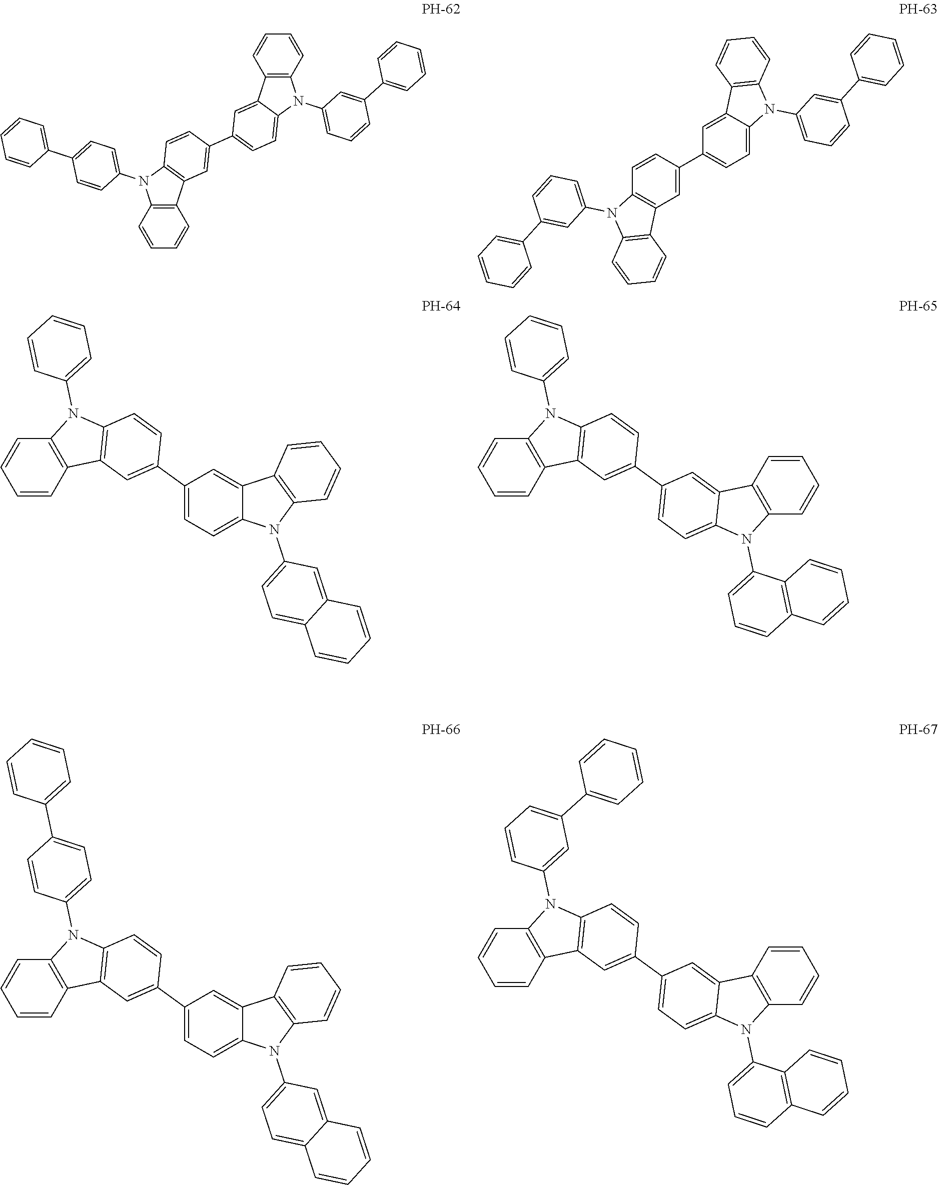

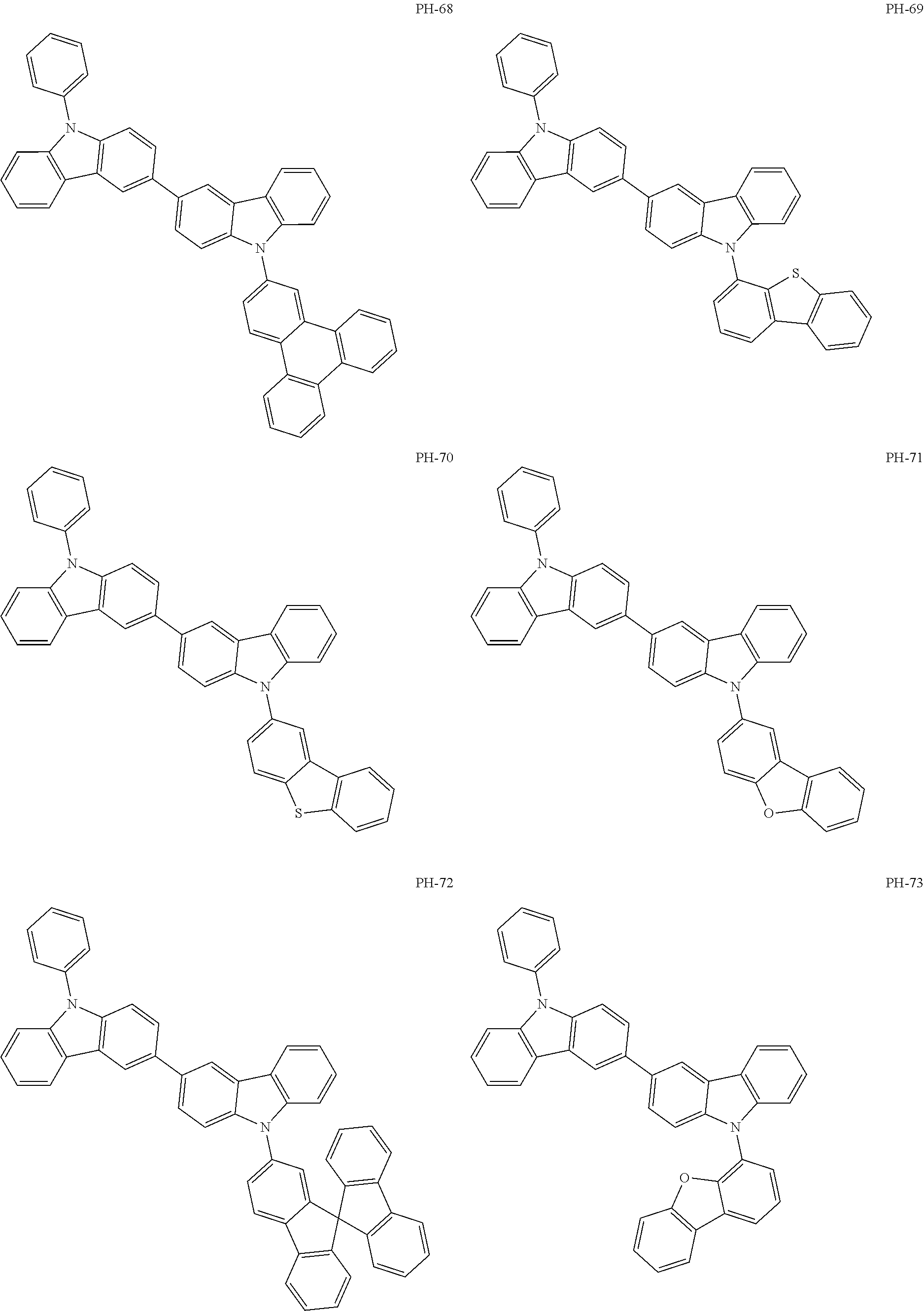 [SMILES: c1ccc(-n2c3ccccc3c3cc(-c4ccc5c(c4)c4ccccc4n5-c4ccc5c6ccccc6c6ccccc6c5c4)ccc32)cc1 ; c1ccc(-n2c3ccccc3c3cc(-c4ccc5c(c4)c4ccccc4n5-c4cccc5c4sc4ccccc45)ccc32)cc1 ; c1ccc(-n2c3ccccc3c3cc(-c4ccc5c(c4)c4ccccc4n5-c4ccc5sc6ccccc6c5c4)ccc32)cc1 ; c1ccc(-n2c3ccccc3c3cc(-c4ccc5c(c4)c4ccccc4n5-c4ccc5oc6ccccc6c5c4)ccc32)cc1 ; c1ccc(-n2c3ccccc3c3cc(-c4ccc5c(c4)c4ccccc4n5-c4ccc5c(c4)C4(c6ccccc6-c6ccccc64)c4ccccc4-5)ccc32)cc1 ; c1ccc(-n2c3ccccc3c3cc(-c4ccc5c(c4)c4ccccc4n5-c4cccc5c4oc4ccccc45)ccc32)cc1]

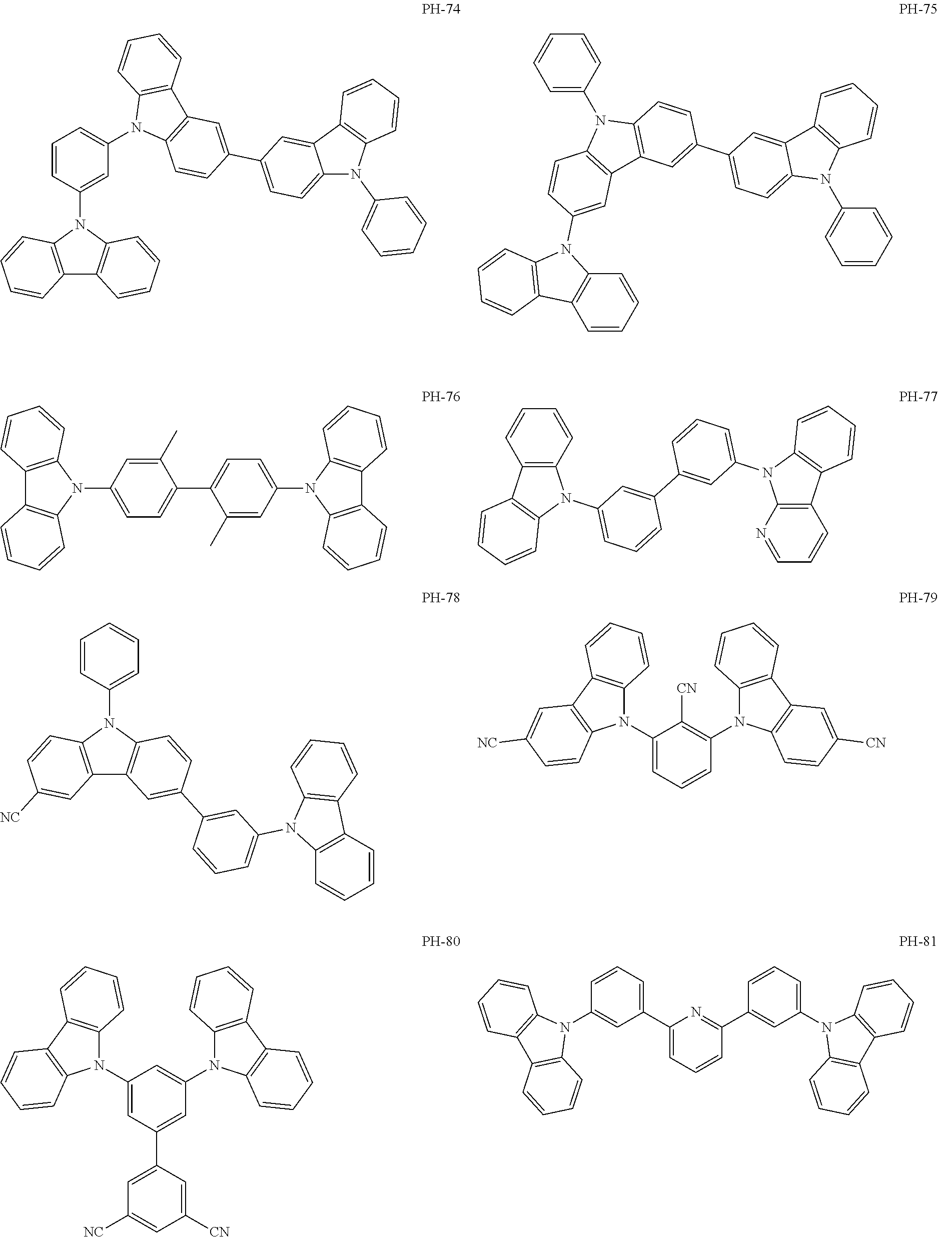

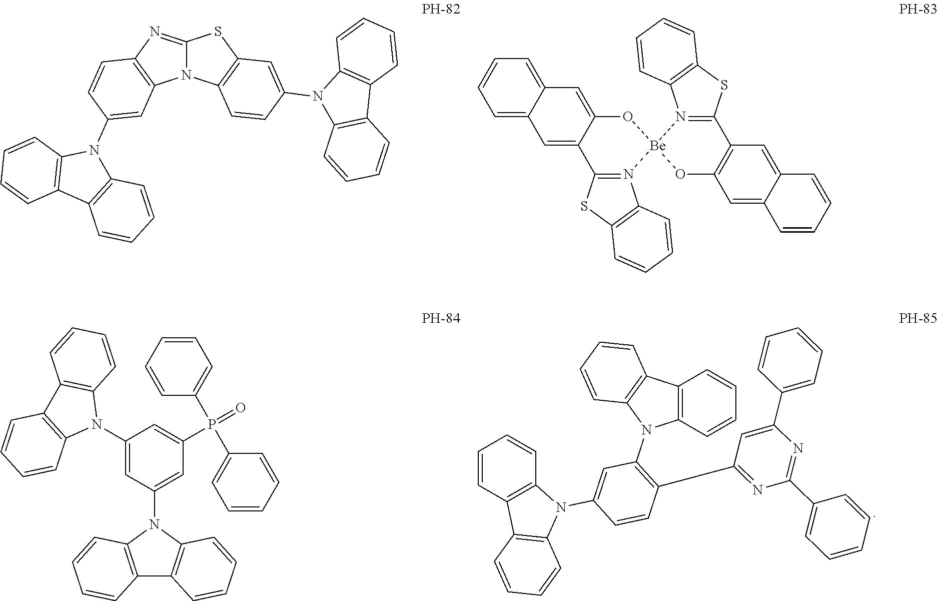

20. (canceled)

21. The organic electroluminescent device according to any one of claim 1, wherein, the thickness of the light emitting layer is 10-65 nm, preferably 15-55 nm;
preferably, the organic layer further comprises any one or a combination of at least two of a hole injection layer, a hole transporting layer, an electron blocking layer, an electron transporting layer or an electron injection layer, wherein the hole injection layer, the hole transporting layer, the electron blocking layer, the light emitting layer, the electron transporting layer and the electron injection layer are arranged in sequence from the anode layer to the cathode layer.

22. (canceled)

23. The organic electroluminescent device according to claim 12, wherein, the first electrode is an anode layer, the second electrode is a con cathode layer, the organic layer contains compound I and compound II, and the compound I is the compound according to claim 1;
the compound II has the structure as shown in Formula (3), 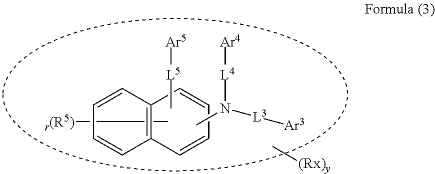
the r is an integer from 0 to 6;
the Ar³ to Ar⁵ are independently selected from the group consisting of a substituted or unsubstituted C6-C30 aryl or a substituted or unsubstituted C3-C30 heteroaryl;
the L³ to L⁵ are each independently selected from the group consisting of any one of a single bond, a substituted or unsubstituted C6-C30 arylene, and a substituted or unsubstituted C3-C30 heteroarylene;
the R⁵ are independently selected from the group consisting of any one of a substituted or unsubstituted C1-C20 chain alkyl, a substituted or unsubstituted C3-C20 cycloalkyl, a substituted or unsubstituted C6-C30 aryl, and a substituted or unsubstituted C3-C30 heteroaryl;
the y is an integer from 1 to 15, the Rx is a substituent at any substitutable position, and at least one Rx is selected from the group consisting of a substituted or unsubstituted C3-C20 cycloalkyl;
In Formula I and Formula (3), in Ar¹ to Ar⁵, L¹ to L⁵, R¹, R², R³, R⁴, R⁵ and Rx, the substituted groups are independently selected from the group consisting of one or a combination of at least two of halogen, C1-C10 chain alkyl, C3-C10 cycloalkyl, C1-C10 alkoxy, C1-C10 thioalkoxy, C6-C30 arylamino, C3-C30 heteroarylamino, C6-C30 monocyclic aryl, C10-C30 fused ring aryl, C3-C30 monocyclic heteroaryl, and C6-C30 fused ring heteroaryl.

24. The organic electroluminescent device according to claim 23, wherein, the compound I is any of the compounds according to claim 11.

25. The organic electroluminescent device according to claim 23, wherein, the compound II has any one of the structures as shown in A1 to A291:

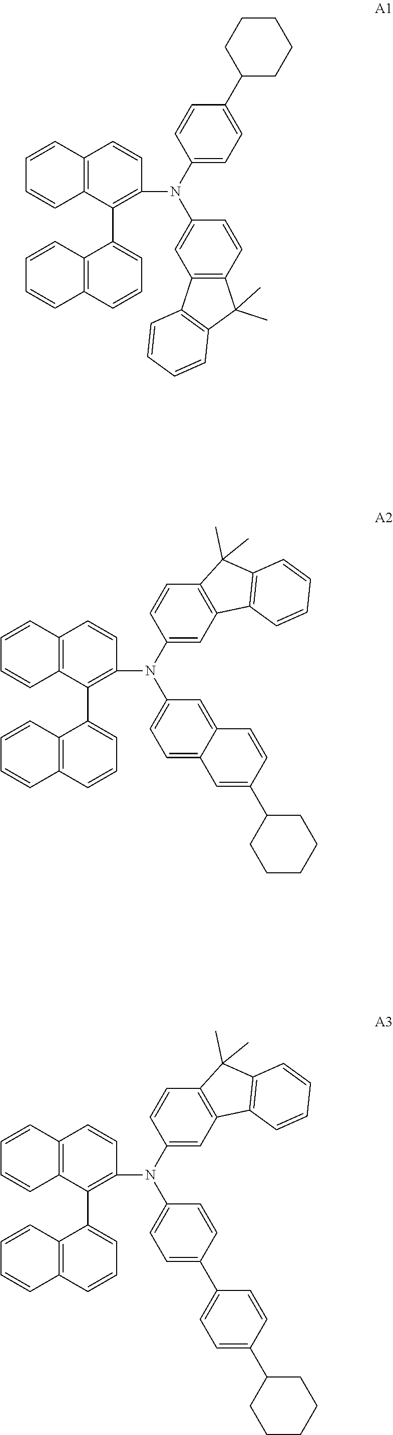

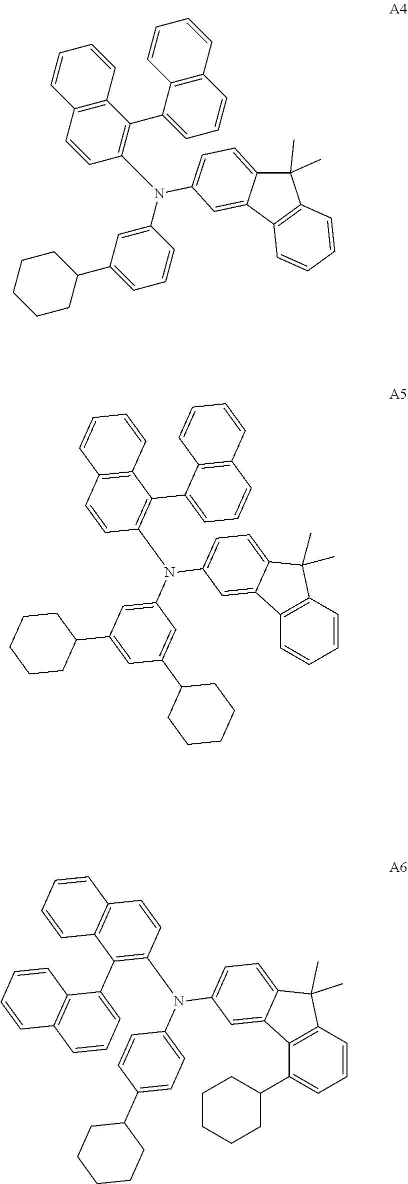 [SMILES: CC1(C)c2ccccc2-c2cc(N(c3cccc(C4CCCCC4)c3)c3ccc4ccccc4c3-c3cccc4ccccc34)ccc21 ; CC1(C)c2ccccc2-c2cc(N(c3cc(C4CCCCC4)cc(C4CCCCC4)c3)c3ccc4ccccc4c3-c3cccc4ccccc34)ccc21 ; CC1(C)c2ccc(N(c3ccc(C4CCCCC4)cc3)c3ccc4ccccc4c3-c3cccc4ccccc34)cc2-c2c(C3CCCCC3)cccc21]

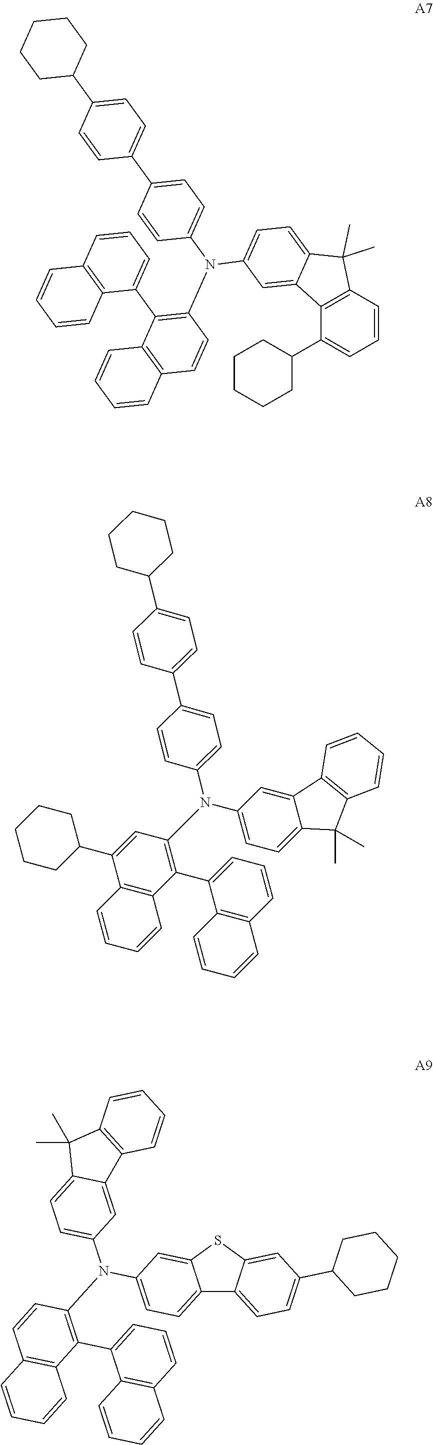 [SMILES: CC1(C)c2ccc(N(c3ccc(-c4ccc(C5CCCCC5)cc4)cc3)c3ccc4ccccc4c3-c3cccc4ccccc34)cc2-c2c(C3CCCCC3)cccc21 ; CC1(C)c2ccccc2-c2cc(N(c3ccc(-c4ccc(C5CCCCC5)cc4)cc3)c3cc(C4CCCCC4)c4ccccc4c3-c3cccc4ccccc34)ccc21 ; CC1(C)c2ccccc2-c2cc(N(c3ccc4c(c3)sc3cc(C5CCCCC5)ccc34)c3ccc4ccccc4c3-c3cccc4ccccc34)ccc21]

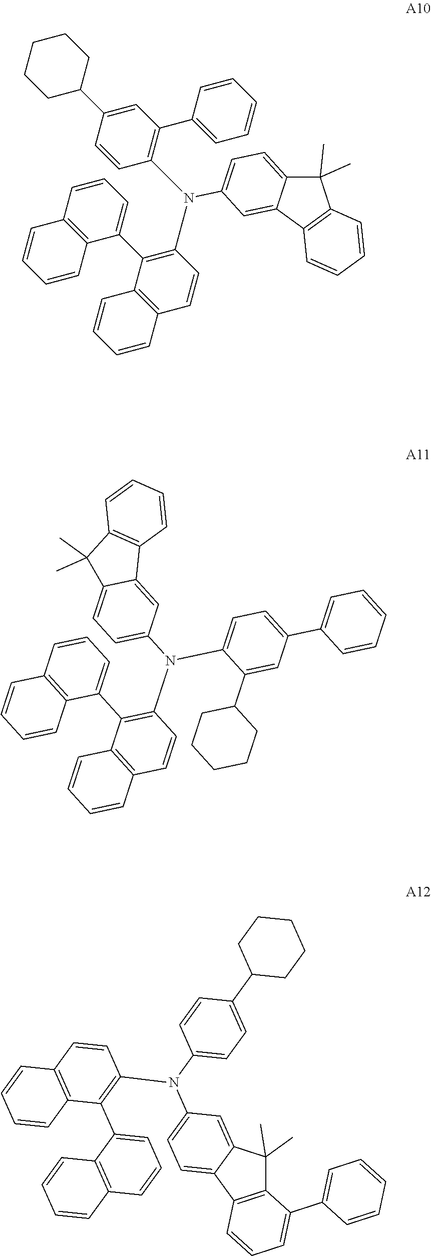 [SMILES: CC1(C)c2ccccc2-c2cc(N(c3ccc(C4CCCCC4)cc3-c3ccccc3)c3ccc4ccccc4c3-c3cccc4ccccc34)ccc21 ; CC1(C)c2ccccc2-c2cc(N(c3ccc(-c4ccccc4)cc3C3CCCCC3)c3ccc4ccccc4c3-c3cccc4ccccc34)ccc21 ; CC1(C)c2cc(N(c3ccc(C4CCCCC4)cc3)c3ccc4ccccc4c3-c3cccc4ccccc34)ccc2-c2cccc(-c3ccccc3)c21]

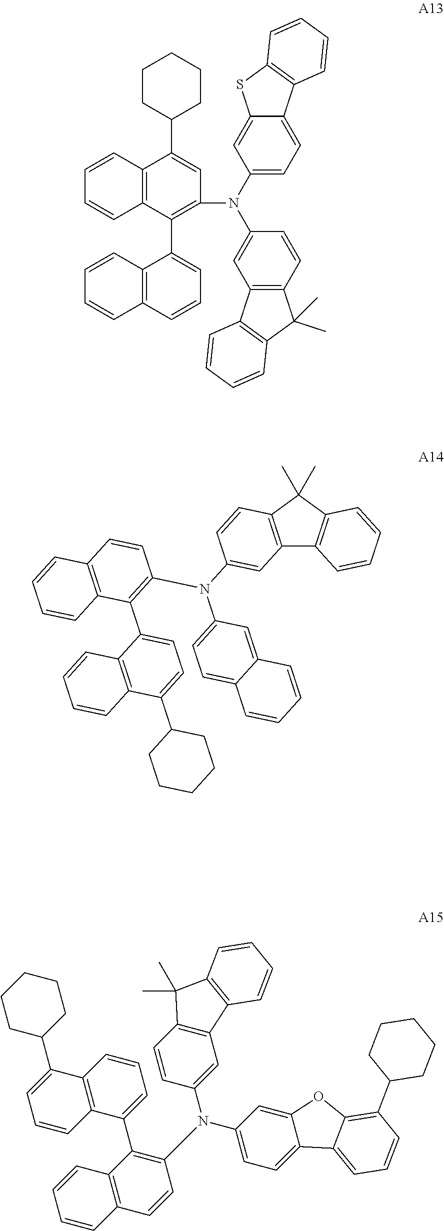 [SMILES: CC1(C)c2ccccc2-c2cc(N(c3ccc4c(c3)sc3ccccc34)c3cc(C4CCCCC4)c4ccccc4c3-c3cccc4ccccc34)ccc21 ; CC1(C)c2ccccc2-c2cc(N(c3ccc4ccccc4c3)c3ccc4ccccc4c3-c3ccc(C4CCCCC4)c4ccccc34)ccc21 ; CC1(C)c2ccccc2-c2cc(N(c3ccc4c(c3)oc3c(C5CCCCC5)cccc34)c3ccc4ccccc4c3-c3cccc4c(C5CCCCC5)cccc34)ccc21]

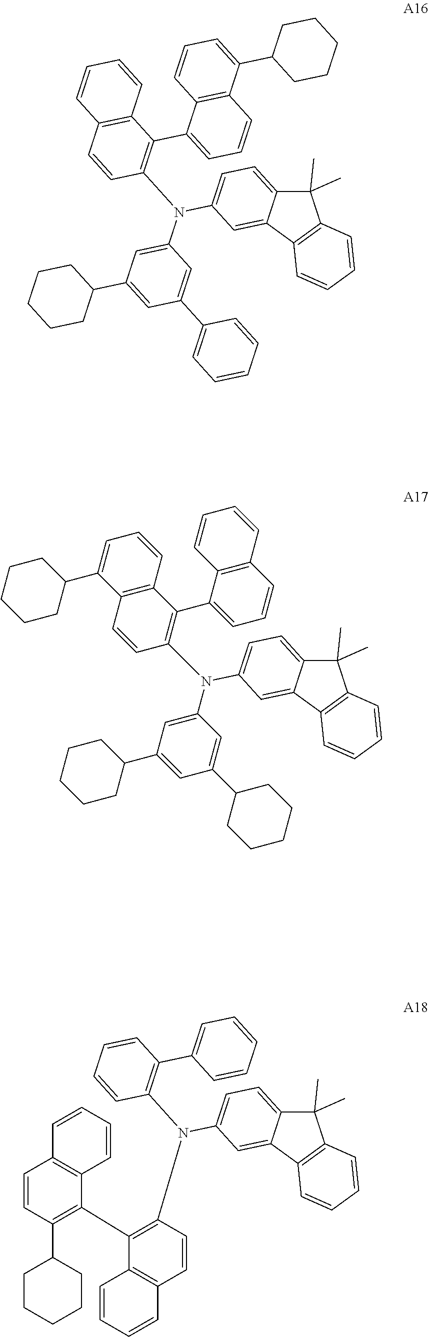 [SMILES: CC1(C)c2ccccc2-c2cc(N(c3cc(-c4ccccc4)cc(C4CCCCC4)c3)c3ccc4ccccc4c3-c3cccc4c(C5CCCCC5)cccc34)ccc21 ; CC1(C)c2ccccc2-c2cc(N(c3cc(C4CCCCC4)cc(C4CCCCC4)c3)c3ccc4c(C5CCCCC5)cccc4c3-c3cccc4ccccc34)ccc21 ; CC1(C)c2ccccc2-c2cc(N(c3ccccc3-c3ccccc3)c3ccc4ccccc4c3-c3c(C4CCCCC4)ccc4ccccc34)ccc21]

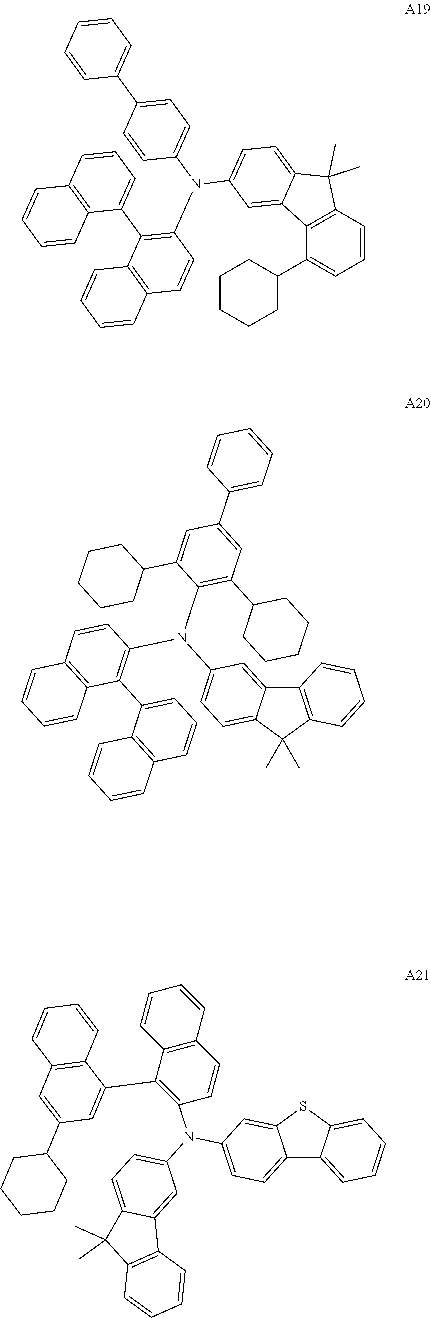 [SMILES: CC1(C)c2ccc(N(c3ccc(-c4ccccc4)cc3)c3ccc4ccccc4c3-c3cccc4ccccc34)cc2-c2c(C3CCCCC3)cccc21 ; CC1(C)c2ccccc2-c2cc(N(c3ccc4ccccc4c3-c3cccc4ccccc34)c3c(C4CCCCC4)cc(-c4ccccc4)cc3C3CCCCC3)ccc21 ; CC1(C)c2ccccc2-c2cc(N(c3ccc4c(c3)sc3ccccc34)c3ccc4ccccc4c3-c3cc(C4CCCCC4)cc4ccccc34)ccc21]

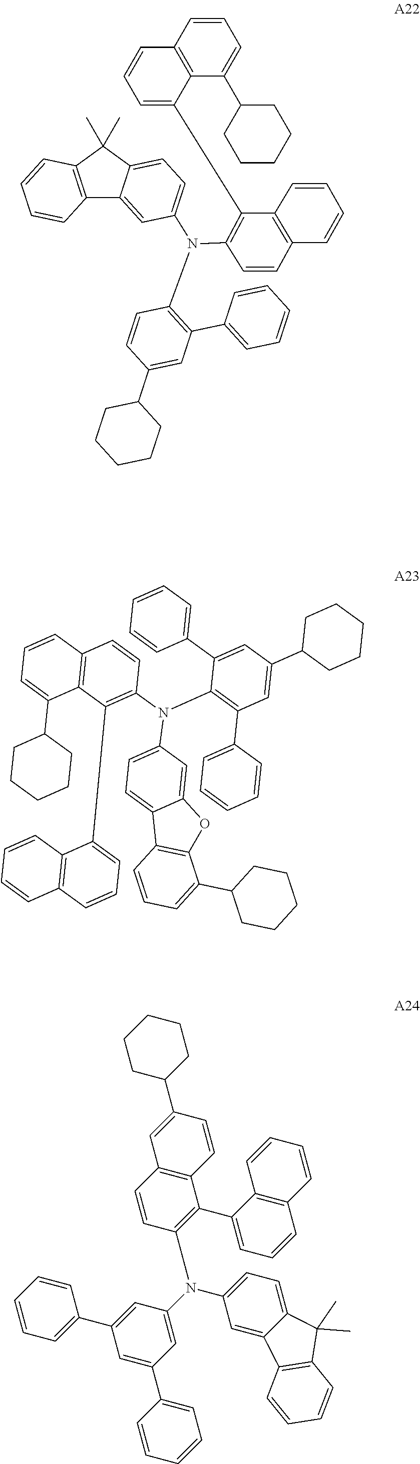 [SMILES: CC1(C)c2ccccc2-c2cc(N(c3ccc(C4CCCCC4)cc3-c3ccccc3)c3ccc4ccccc4c3-c3cccc4cccc(C5CCCCC5)c34)ccc21 ; c1ccc(-c2cc(C3CCCCC3)cc(-c3ccccc3)c2N(c2ccc3c(c2)oc2c(C4CCCCC4)cccc23)c2ccc3cccc(C4CCCCC4)c3c2-c2cccc3ccccc23)cc1 ; CC1(C)c2ccccc2-c2cc(N(c3cc(-c4ccccc4)cc(-c4ccccc4)c3)c3ccc4cc(C5CCCCC5)ccc4c3-c3cccc4ccccc34)ccc21]

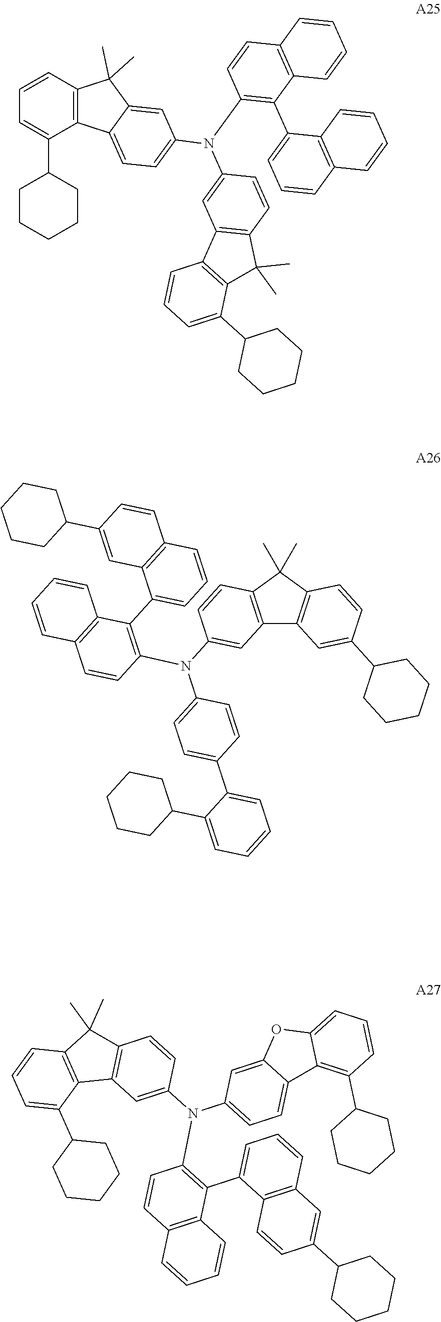 [SMILES: CC1(C)c2cc(N(c3ccc4c(c3)-c3cccc(C5CCCCC5)c3C4(C)C)c3ccc4ccccc4c3-c3cccc4ccccc34)ccc2-c2c(C3CCCCC3)cccc21 ; CC1(C)c2ccc(C3CCCCC3)cc2-c2cc(N(c3ccc(-c4ccccc4C4CCCCC4)cc3)c3ccc4ccccc4c3-c3cccc4ccc(C5CCCCC5)cc34)ccc21 ; CC1(C)c2ccc(N(c3ccc4c(c3)oc3cccc(C5CCCCC5)c34)c3ccc4ccccc4c3-c3cccc4cc(C5CCCCC5)ccc34)cc2-c2c(C3CCCCC3)cccc21]

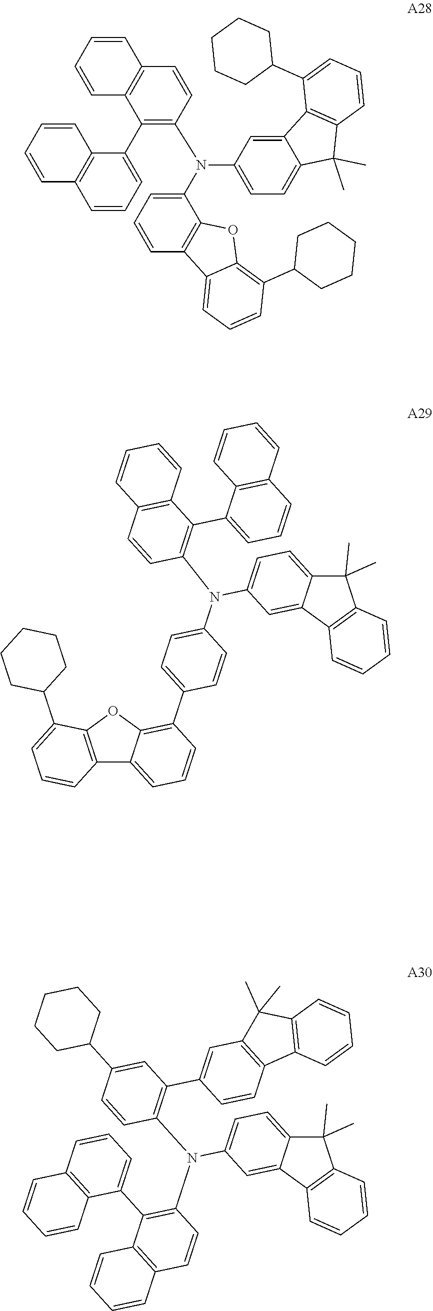 [SMILES: CC1(C)c2ccc(N(c3ccc4ccccc4c3-c3cccc4ccccc34)c3cccc4c3oc3c(C5CCCCC5)cccc34)cc2-c2c(C3CCCCC3)cccc21 ; CC1(C)c2ccccc2-c2cc(N(c3ccc(-c4cccc5c4oc4c(C6CCCCC6)cccc45)cc3)c3ccc4ccccc4c3-c3cccc4ccccc34)ccc21 ; CC1(C)c2ccccc2-c2cc(N(c3ccc(C4CCCCC4)cc3-c3ccc4c(c3)C(C)(C)c3ccccc3-4)c3ccc4ccccc4c3-c3cccc4ccccc34)ccc21]

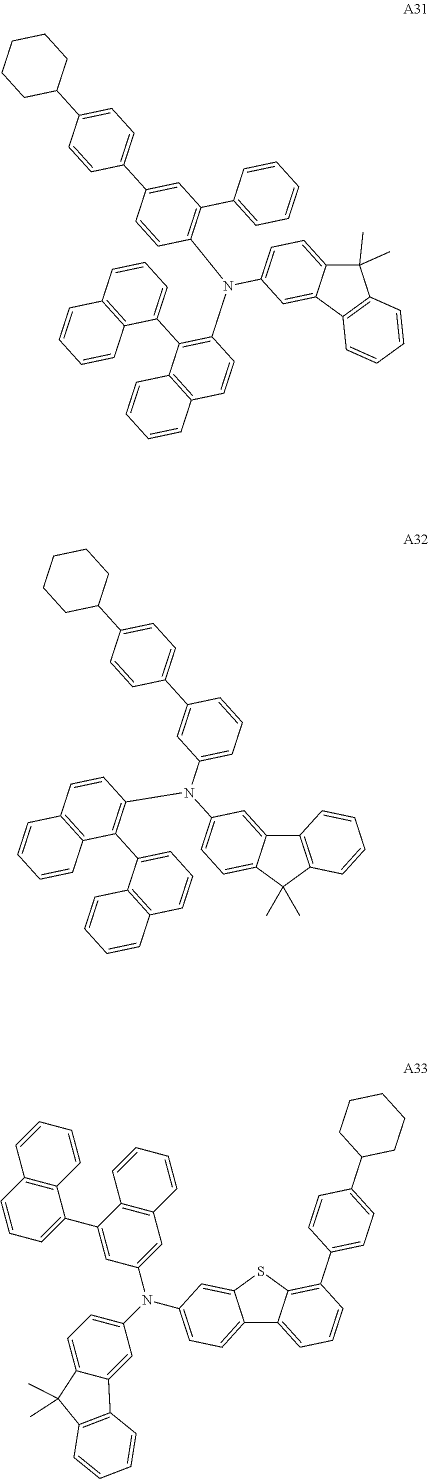 [SMILES: CC1(C)c2ccccc2-c2cc(N(c3ccc(-c4ccc(C5CCCCC5)cc4)cc3-c3ccccc3)c3ccc4ccccc4c3-c3cccc4ccccc34)ccc21 ; CC1(C)c2ccccc2-c2cc(N(c3cccc(-c4ccc(C5CCCCC5)cc4)c3)c3ccc4ccccc4c3-c3cccc4ccccc34)ccc21 ; CC1(C)c2ccccc2-c2cc(N(c3cc(-c4cccc5ccccc45)c4ccccc4c3)c3ccc4c(c3)sc3c(-c5ccc(C6CCCCC6)cc5)cccc34)ccc21]

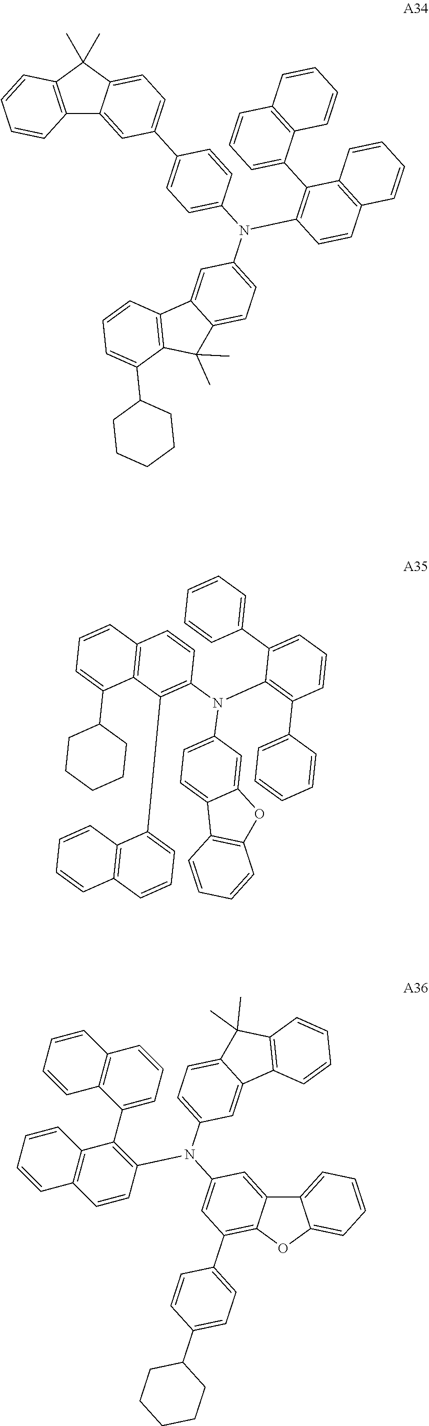 [SMILES: CC1(C)c2ccccc2-c2cc(-c3ccc(N(c4ccc5c(c4)-c4cccc(C6CCCCC6)c4C5(C)C)c4ccc5ccccc5c4-c4cccc5ccccc45)cc3)ccc21 ; c1ccc(-c2cccc(-c3ccccc3)c2N(c2ccc3c(c2)oc2ccccc23)c2ccc3cccc(C4CCCCC4)c3c2-c2cccc3ccccc23)cc1 ; CC1(C)c2ccccc2-c2cc(N(c3cc(-c4ccc(C5CCCCC5)cc4)c4oc5ccccc5c4c3)c3ccc4ccccc4c3-c3cccc4ccccc34)ccc21]

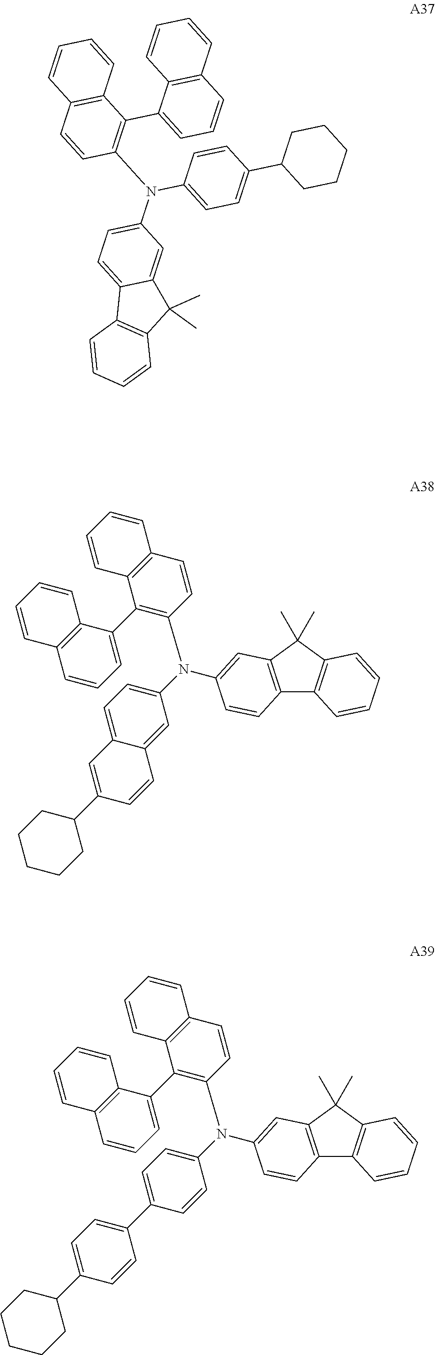 [SMILES: CC1(C)c2ccccc2-c2ccc(N(c3ccc(C4CCCCC4)cc3)c3ccc4ccccc4c3-c3cccc4ccccc34)cc21 ; CC1(C)c2ccccc2-c2ccc(N(c3ccc4cc(C5CCCCC5)ccc4c3)c3ccc4ccccc4c3-c3cccc4ccccc34)cc21 ; CC1(C)c2ccccc2-c2ccc(N(c3ccc(-c4ccc(C5CCCCC5)cc4)cc3)c3ccc4ccccc4c3-c3cccc4ccccc34)cc21]

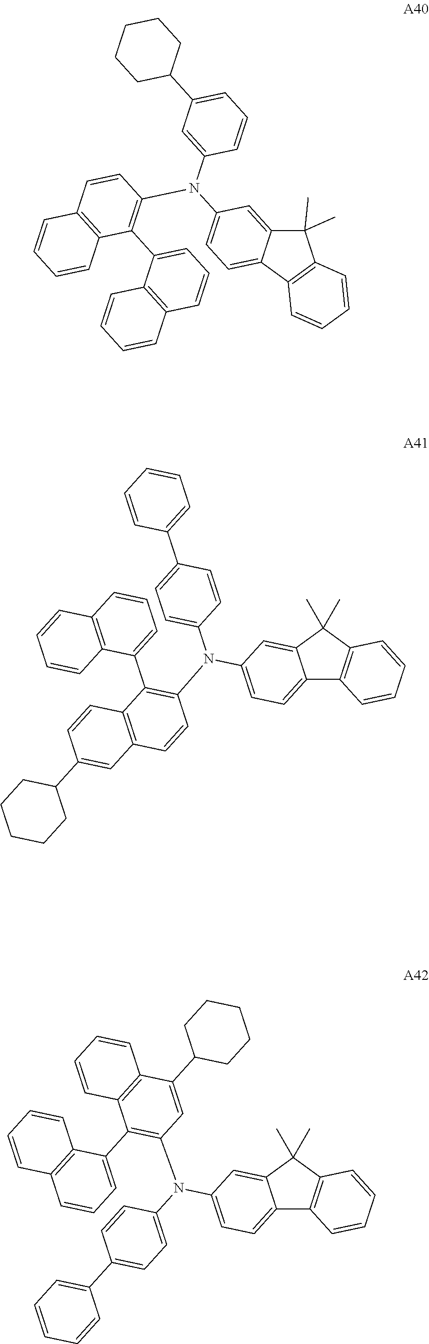 [SMILES: CC1(C)c2ccccc2-c2ccc(N(c3cccc(C4CCCCC4)c3)c3ccc4ccccc4c3-c3cccc4ccccc34)cc21 ; CC1(C)c2ccccc2-c2ccc(N(c3ccc(-c4ccccc4)cc3)c3ccc4cc(C5CCCCC5)ccc4c3-c3cccc4ccccc34)cc21 ; CC1(C)c2ccccc2-c2ccc(N(c3ccc(-c4ccccc4)cc3)c3cc(C4CCCCC4)c4ccccc4c3-c3cccc4ccccc34)cc21]

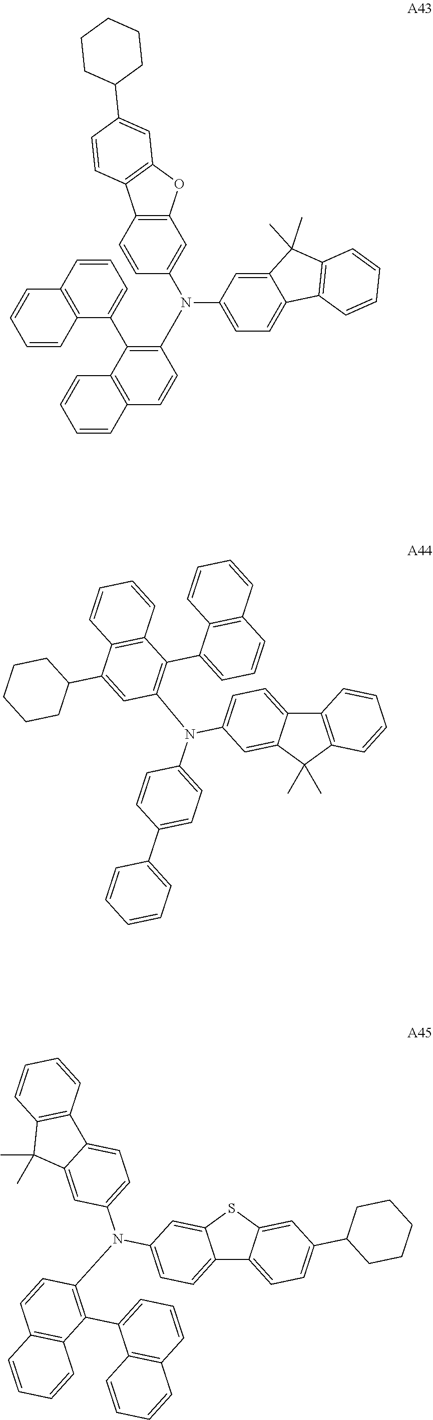 [SMILES: CC1(C)c2ccccc2-c2ccc(N(c3ccc4c(c3)oc3cc(C5CCCCC5)ccc34)c3ccc4ccccc4c3-c3cccc4ccccc34)cc21 ; CC1(C)c2ccccc2-c2ccc(N(c3ccc(-c4ccccc4)cc3)c3cc(C4CCCCC4)c4ccccc4c3-c3cccc4ccccc34)cc21 ; CC1(C)c2ccccc2-c2ccc(N(c3ccc4c(c3)sc3cc(C5CCCCC5)ccc34)c3ccc4ccccc4c3-c3cccc4ccccc34)cc21]

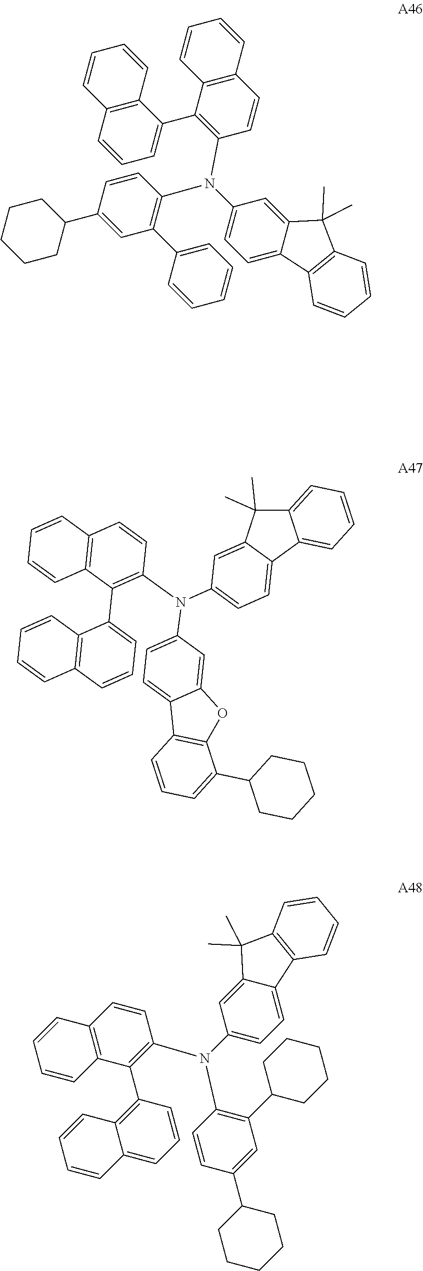 [SMILES: CC1(C)c2ccccc2-c2ccc(N(c3ccc(C4CCCCC4)cc3-c3ccccc3)c3ccc4ccccc4c3-c3cccc4ccccc34)cc21 ; CC1(C)c2ccccc2-c2ccc(N(c3ccc4c(c3)oc3c(C5CCCCC5)cccc34)c3ccc4ccccc4c3-c3cccc4ccccc34)cc21 ; CC1(C)c2ccccc2-c2ccc(N(c3ccc(C4CCCCC4)cc3C3CCCCC3)c3ccc4ccccc4c3-c3cccc4ccccc34)cc21]

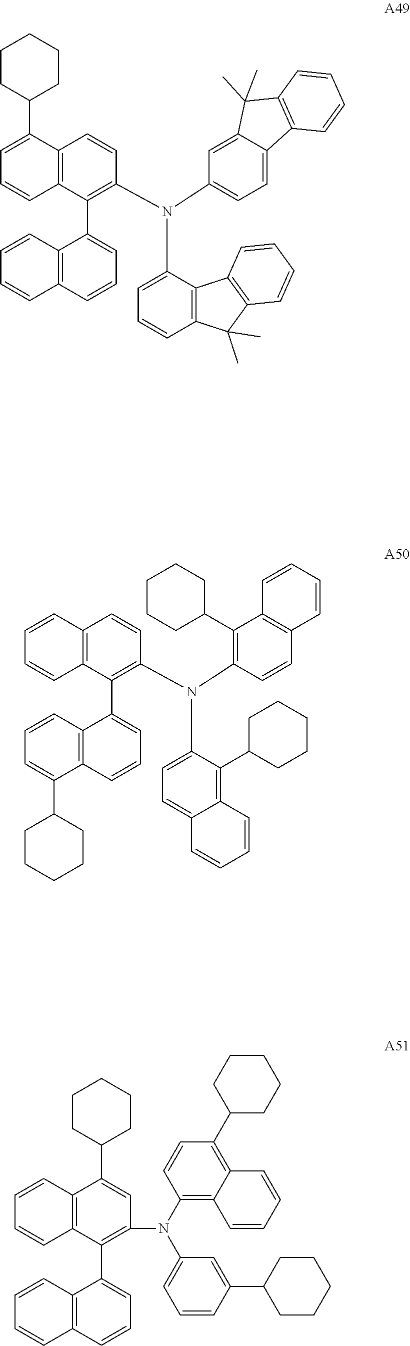 [SMILES: CC1(C)c2ccccc2-c2ccc(N(c3cccc4c3-c3ccccc3C4(C)C)c3ccc4c(C5CCCCC5)cccc4c3-c3cccc4ccccc34)cc21 ; c1ccc2c(-c3cccc4c(C5CCCCC5)cccc34)c(N(c3ccc4ccccc4c3C3CCCCC3)c3ccc4ccccc4c3C3CCCCC3)ccc2c1 ; c1cc(C2CCCCC2)cc(N(c2cc(C3CCCCC3)c3ccccc3c2-c2cccc3ccccc23)c2ccc(C3CCCCC3)c3ccccc23)c1]

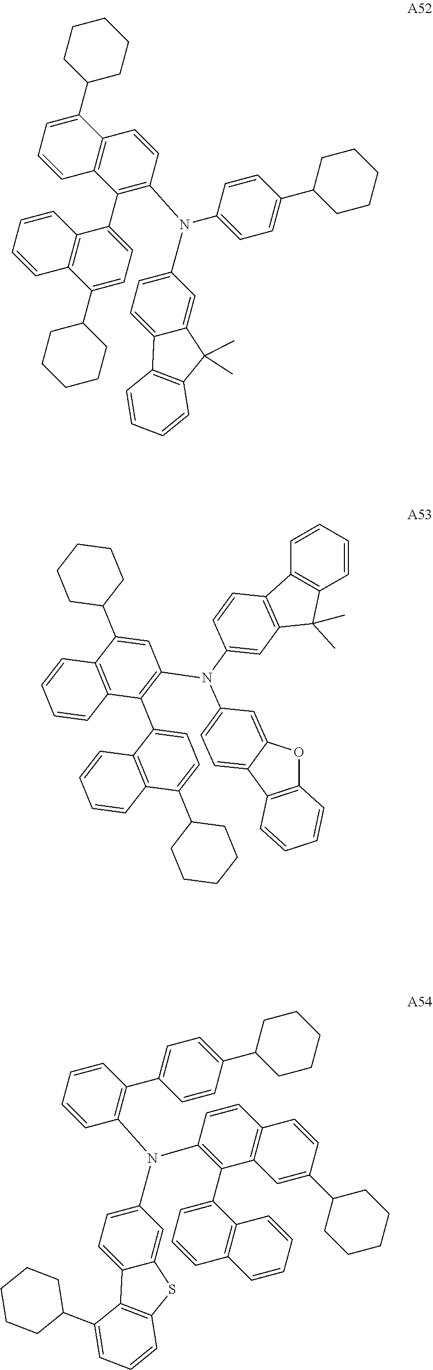 [SMILES: CC1(C)c2ccccc2-c2ccc(N(c3ccc(C4CCCCC4)cc3)c3ccc4c(C5CCCCC5)cccc4c3-c3ccc(C4CCCCC4)c4ccccc34)cc21 ; CC1(C)c2ccccc2-c2ccc(N(c3ccc4c(c3)oc3ccccc34)c3cc(C4CCCCC4)c4ccccc4c3-c3ccc(C4CCCCC4)c4ccccc34)cc21 ; c1ccc(N(c2ccc3c(c2)sc2cccc(C4CCCCC4)c23)c2ccc3ccc(C4CCCCC4)cc3c2-c2cccc3ccccc23)c(-c2ccc(C3CCCCC3)cc2)c1]

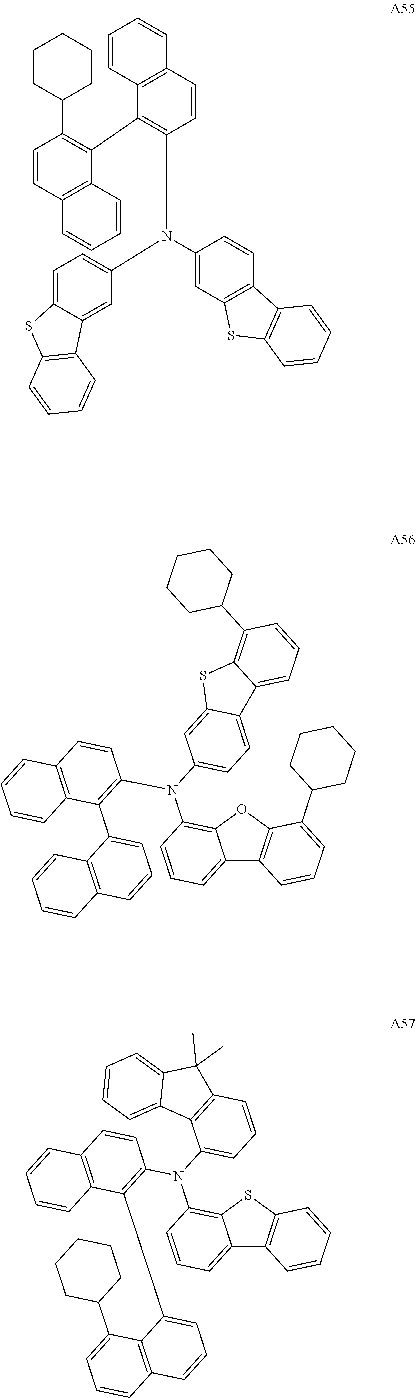 [SMILES: c1ccc2c(-c3c(N(c4ccc5c(c4)sc4ccccc45)c4ccc5sc6ccccc6c5c4)ccc4ccccc34)c(C3CCCCC3)ccc2c1 ; c1ccc2c(-c3c(N(c4ccc5c(c4)sc4c(C6CCCCC6)cccc45)c4cccc5c4oc4c(C6CCCCC6)cccc45)ccc4ccccc34)cccc2c1 ; CC1(C)c2ccccc2-c2c(N(c3ccc4ccccc4c3-c3cccc4cccc(C5CCCCC5)c34)c3cccc4c3sc3ccccc34)cccc21]

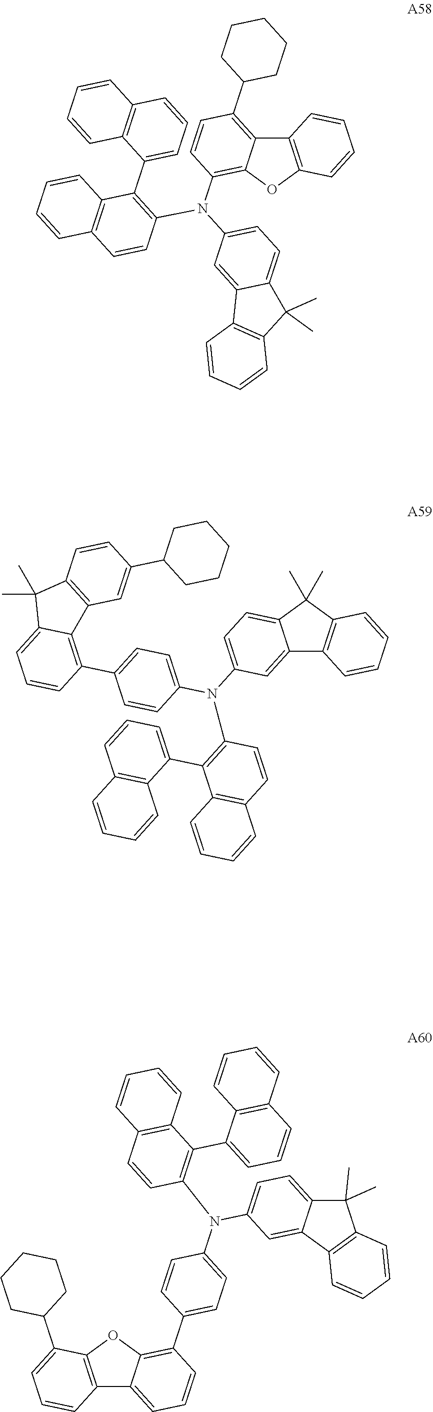 [SMILES: CC1(C)c2ccccc2-c2cc(N(c3ccc4ccccc4c3-c3cccc4ccccc34)c3ccc(C4CCCCC4)c4c3oc3ccccc34)ccc21 ; CC1(C)c2ccccc2-c2cc(N(c3ccc(-c4cccc5c4-c4cc(C6CCCCC6)ccc4C5(C)C)cc3)c3ccc4ccccc4c3-c3cccc4ccccc34)ccc21 ; CC1(C)c2ccccc2-c2cc(N(c3ccc(-c4cccc5c4oc4c(C6CCCCC6)cccc45)cc3)c3ccc4ccccc4c3-c3cccc4ccccc34)ccc21]

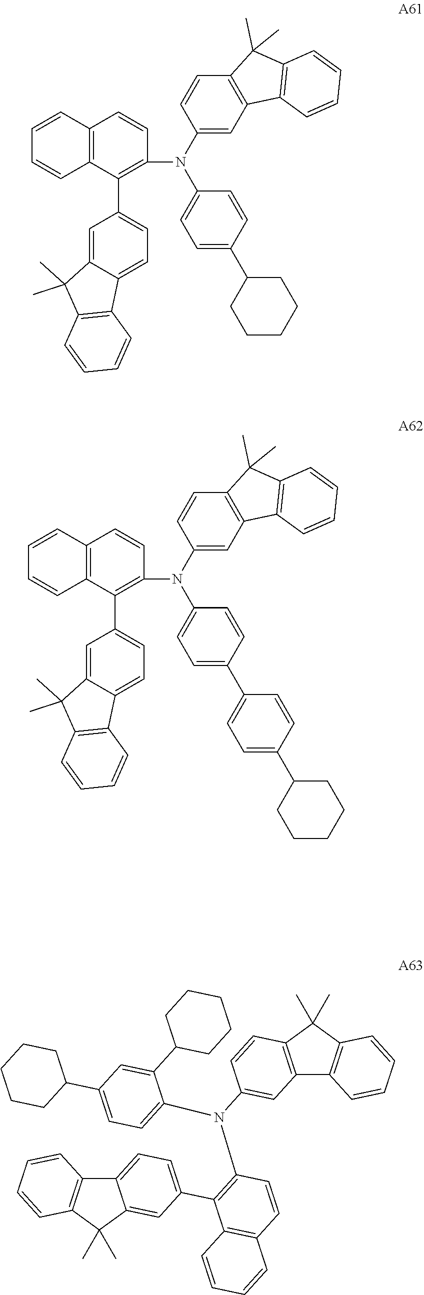 [SMILES: CC1(C)c2ccccc2-c2cc(N(c3ccc(C4CCCCC4)cc3)c3ccc4ccccc4c3-c3ccc4c(c3)C(C)(C)c3ccccc3-4)ccc21 ; CC1(C)c2ccccc2-c2cc(N(c3ccc(-c4ccc(C5CCCCC5)cc4)cc3)c3ccc4ccccc4c3-c3ccc4c(c3)C(C)(C)c3ccccc3-4)ccc21 ; CC1(C)c2ccccc2-c2cc(N(c3ccc(C4CCCCC4)cc3C3CCCCC3)c3ccc4ccccc4c3-c3ccc4c(c3)C(C)(C)c3ccccc3-4)ccc21]

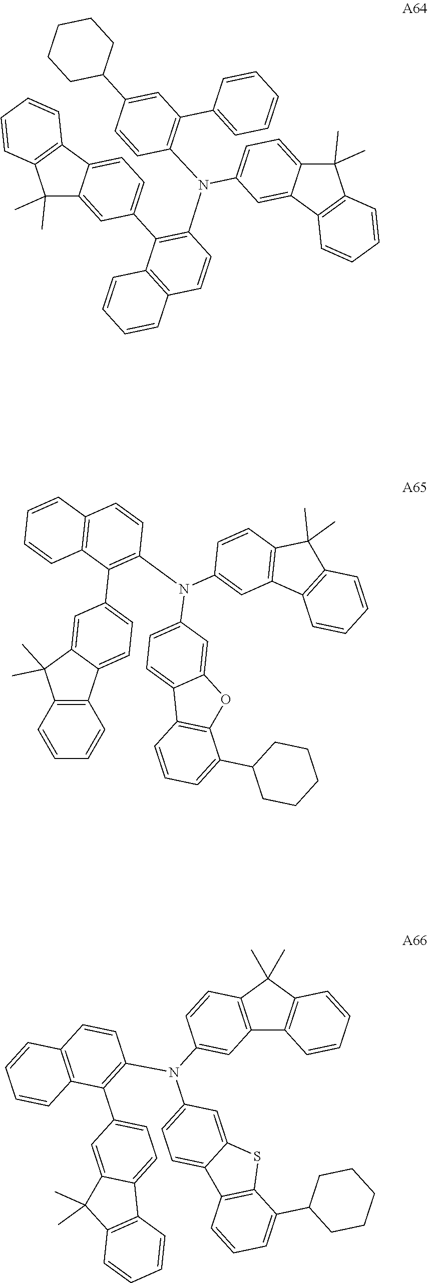 [SMILES: CC1(C)c2ccccc2-c2cc(N(c3ccc(C4CCCCC4)cc3-c3ccccc3)c3ccc4ccccc4c3-c3ccc4c(c3)C(C)(C)c3ccccc3-4)ccc21 ; CC1(C)c2ccccc2-c2cc(N(c3ccc4c(c3)oc3c(C5CCCCC5)cccc34)c3ccc4ccccc4c3-c3ccc4c(c3)C(C)(C)c3ccccc3-4)ccc21 ; CC1(C)c2ccccc2-c2cc(N(c3ccc4c(c3)sc3c(C5CCCCC5)cccc34)c3ccc4ccccc4c3-c3ccc4c(c3)C(C)(C)c3ccccc3-4)ccc21]

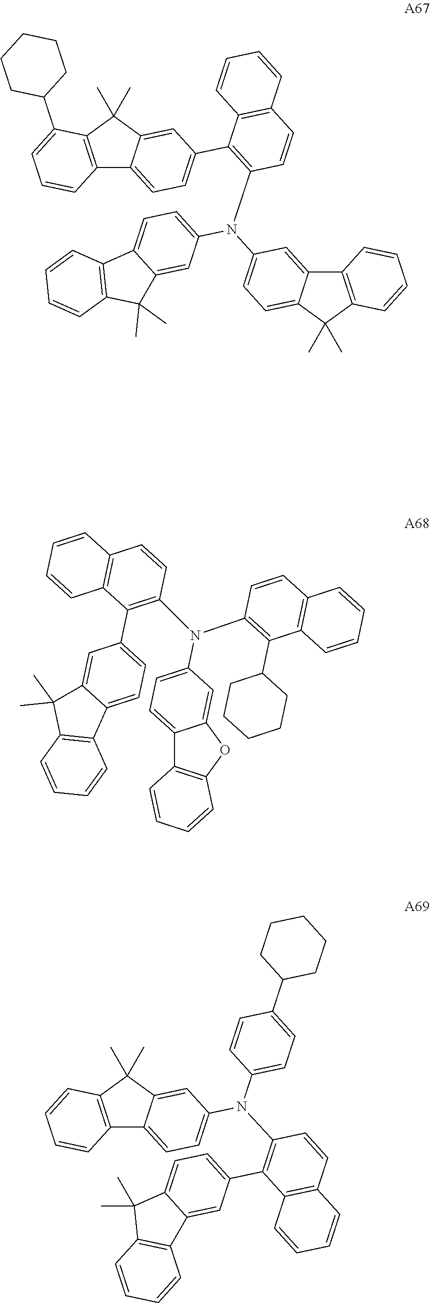 [SMILES: CC1(C)c2ccccc2-c2cc(N(c3ccc4c(c3)C(C)(C)c3ccccc3-4)c3ccc4ccccc4c3-c3ccc4c(c3)C(C)(C)c3c-4cccc3C3CCCCC3)ccc21 ; CC1(C)c2ccccc2-c2ccc(-c3c(N(c4ccc5c(c4)oc4ccccc45)c4ccc5ccccc5c4C4CCCCC4)ccc4ccccc34)cc21 ; CC1(C)c2ccccc2-c2cc(-c3c(N(c4ccc(C5CCCCC5)cc4)c4ccc5c(c4)C(C)(C)c4ccccc4-5)ccc4ccccc34)ccc21]

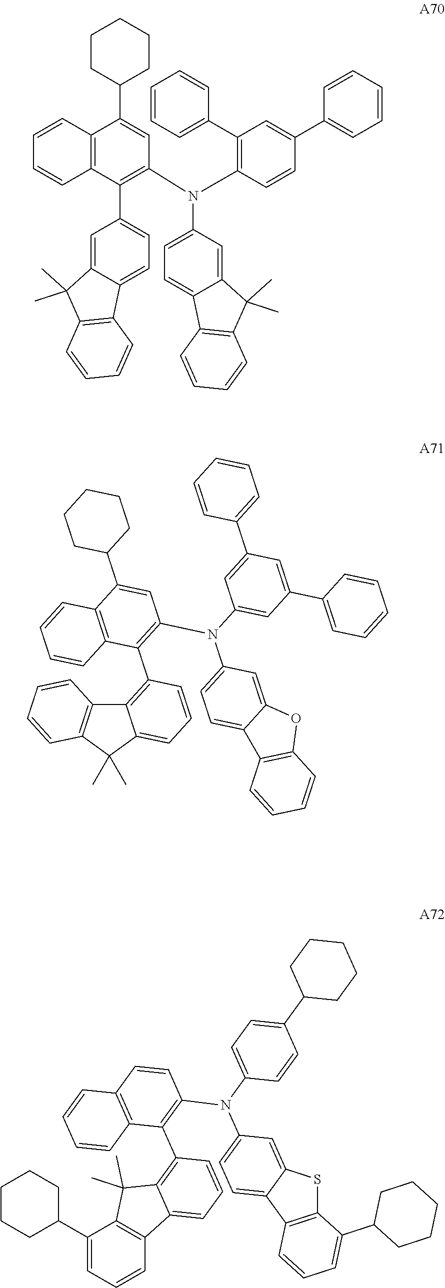 [SMILES: CC1(C)c2ccccc2-c2ccc(-c3c(N(c4ccc5c(c4)C(C)(C)c4ccccc4-5)c4ccc(-c5ccccc5)cc4-c4ccccc4)cc(C4CCCCC4)c4ccccc34)cc21 ; CC1(C)c2ccccc2-c2c(-c3c(N(c4cc(-c5ccccc5)cc(-c5ccccc5)c4)c4ccc5c(c4)oc4ccccc45)cc(C4CCCCC4)c4ccccc34)cccc21 ; CC1(C)c2c(cccc2-c2c(N(c3ccc(C4CCCCC4)cc3)c3ccc4c(c3)sc3c(C5CCCCC5)cccc34)ccc3ccccc23)-c2cccc(C3CCCCC3)c21]

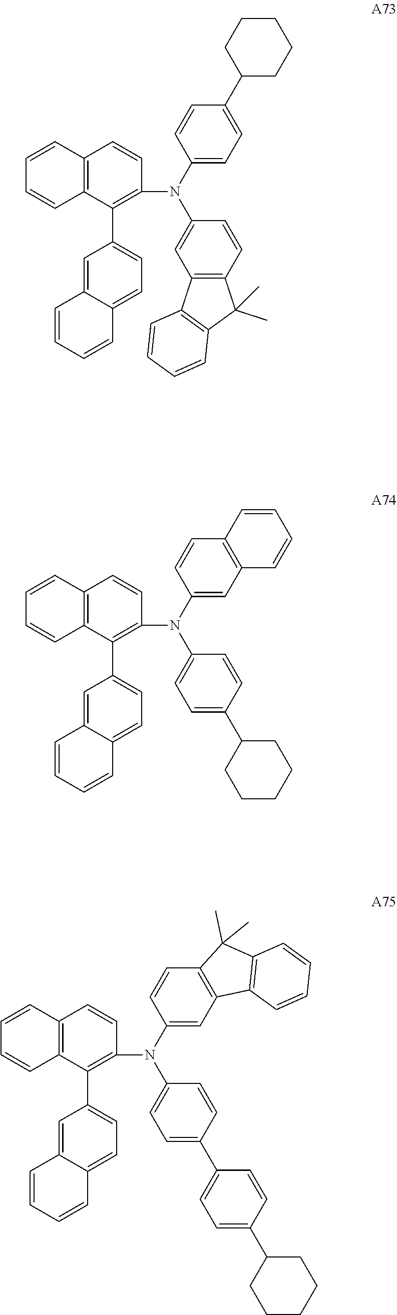 [SMILES: CC1(C)c2ccccc2-c2cc(N(c3ccc(C4CCCCC4)cc3)c3ccc4ccccc4c3-c3ccc4ccccc4c3)ccc21 ; c1ccc2cc(-c3c(N(c4ccc(C5CCCCC5)cc4)c4ccc5ccccc5c4)ccc4ccccc34)ccc2c1 ; CC1(C)c2ccccc2-c2cc(N(c3ccc(-c4ccc(C5CCCCC5)cc4)cc3)c3ccc4ccccc4c3-c3ccc4ccccc4c3)ccc21]

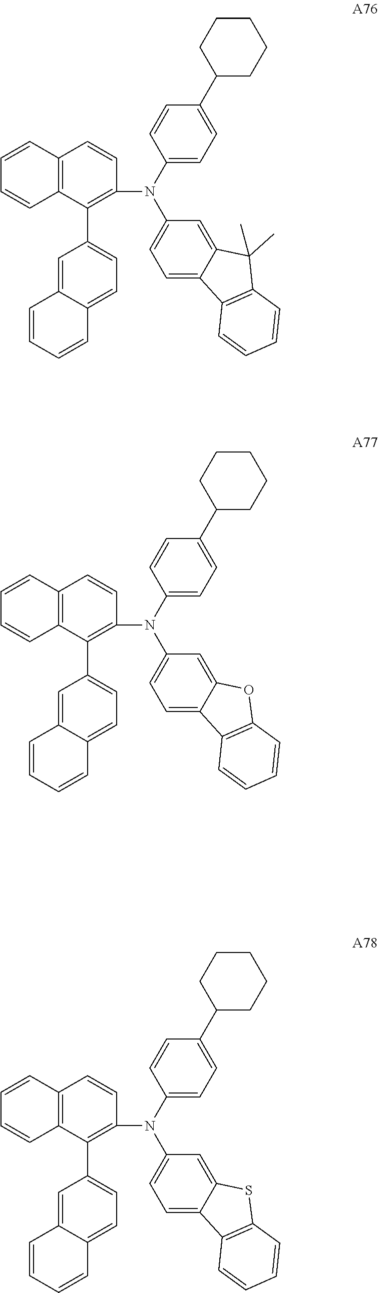 [SMILES: CC1(C)c2ccccc2-c2ccc(N(c3ccc(C4CCCCC4)cc3)c3ccc4ccccc4c3-c3ccc4ccccc4c3)cc21 ; c1ccc2cc(-c3c(N(c4ccc(C5CCCCC5)cc4)c4ccc5c(c4)oc4ccccc45)ccc4ccccc34)ccc2c1 ; c1ccc2cc(-c3c(N(c4ccc(C5CCCCC5)cc4)c4ccc5c(c4)sc4ccccc45)ccc4ccccc34)ccc2c1]

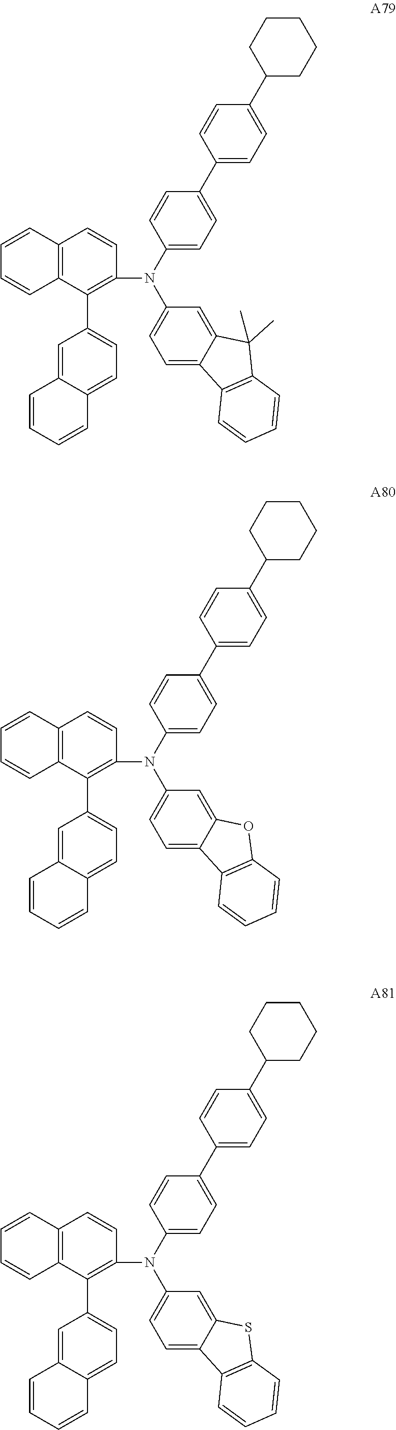 [SMILES: CC1(C)c2ccccc2-c2ccc(N(c3ccc(-c4ccc(C5CCCCC5)cc4)cc3)c3ccc4ccccc4c3-c3ccc4ccccc4c3)cc21 ; c1ccc2cc(-c3c(N(c4ccc(-c5ccc(C6CCCCC6)cc5)cc4)c4ccc5c(c4)oc4ccccc45)ccc4ccccc34)ccc2c1 ; c1ccc2cc(-c3c(N(c4ccc(-c5ccc(C6CCCCC6)cc5)cc4)c4ccc5c(c4)sc4ccccc45)ccc4ccccc34)ccc2c1]

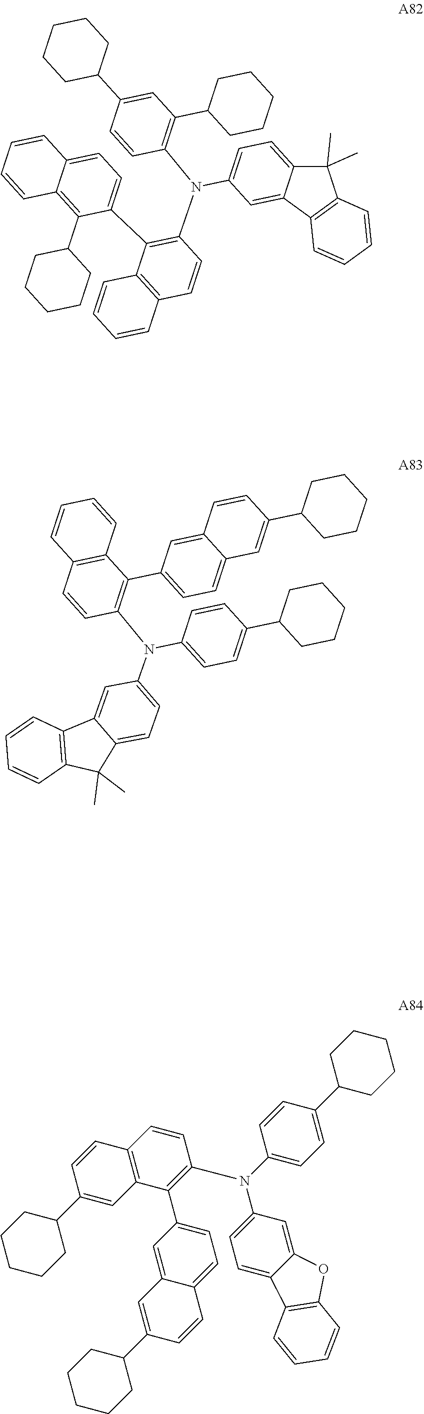 [SMILES: CC1(C)c2ccccc2-c2cc(N(c3ccc(C4CCCCC4)cc3C3CCCCC3)c3ccc4ccccc4c3-c3ccc4ccccc4c3C3CCCCC3)ccc21 ; CC1(C)c2ccccc2-c2cc(N(c3ccc(C4CCCCC4)cc3)c3ccc4ccccc4c3-c3ccc4cc(C5CCCCC5)ccc4c3)ccc21 ; c1ccc2c(c1)oc1cc(N(c3ccc(C4CCCCC4)cc3)c3ccc4ccc(C5CCCCC5)cc4c3-c3ccc4ccc(C5CCCCC5)cc4c3)ccc12]

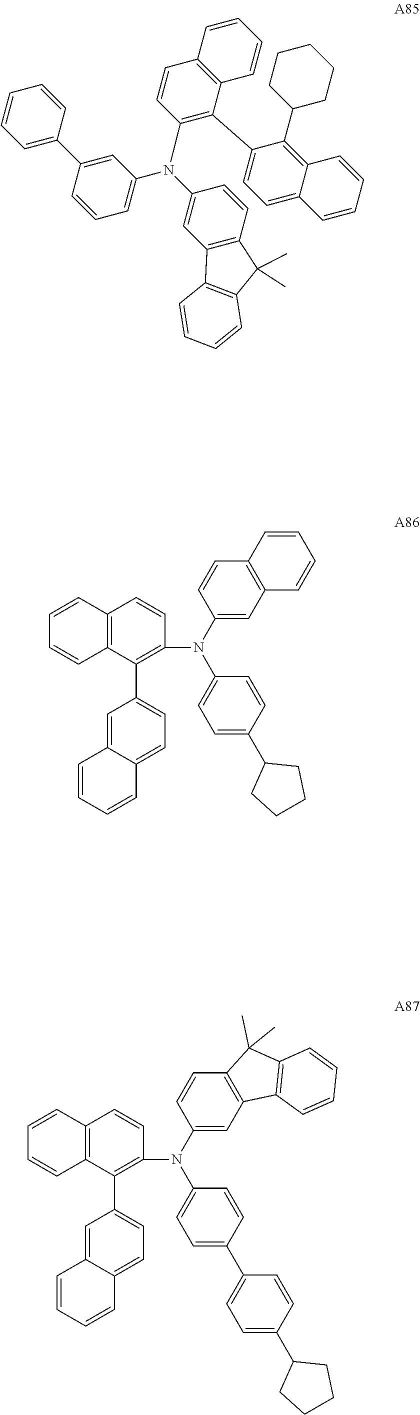 [SMILES: CC1(C)c2ccccc2-c2cc(N(c3cccc(-c4ccccc4)c3)c3ccc4ccccc4c3-c3ccc4ccccc4c3C3CCCCC3)ccc21 ; c1ccc2cc(-c3c(N(c4ccc(C5CCCC5)cc4)c4ccc5ccccc5c4)ccc4ccccc34)ccc2c1 ; CC1(C)c2ccccc2-c2cc(N(c3ccc(-c4ccc(C5CCCC5)cc4)cc3)c3ccc4ccccc4c3-c3ccc4ccccc4c3)ccc21]

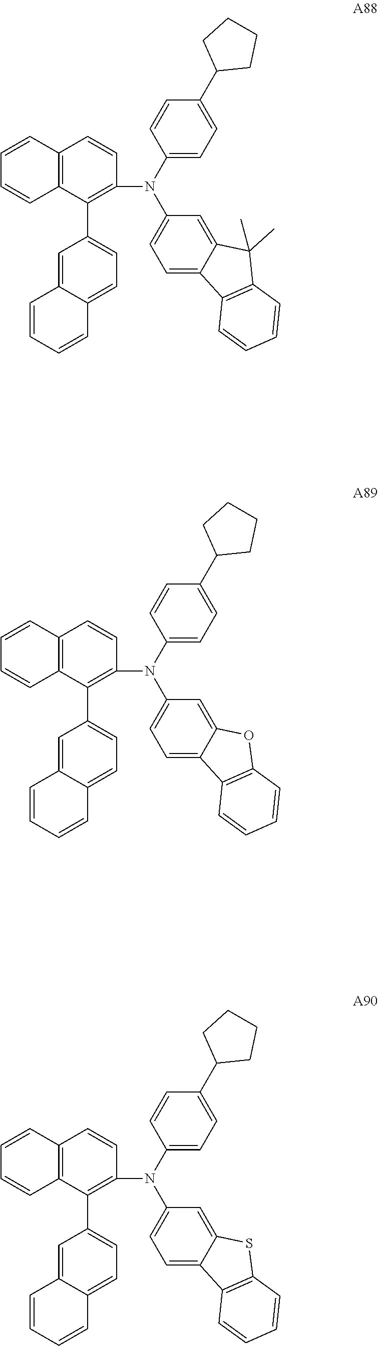 [SMILES: CC1(C)c2ccccc2-c2ccc(N(c3ccc(C4CCCC4)cc3)c3ccc4ccccc4c3-c3ccc4ccccc4c3)cc21 ; c1ccc2cc(-c3c(N(c4ccc(C5CCCC5)cc4)c4ccc5c(c4)oc4ccccc45)ccc4ccccc34)ccc2c1 ; c1ccc2cc(-c3c(N(c4ccc(C5CCCC5)cc4)c4ccc5c(c4)sc4ccccc45)ccc4ccccc34)ccc2c1]

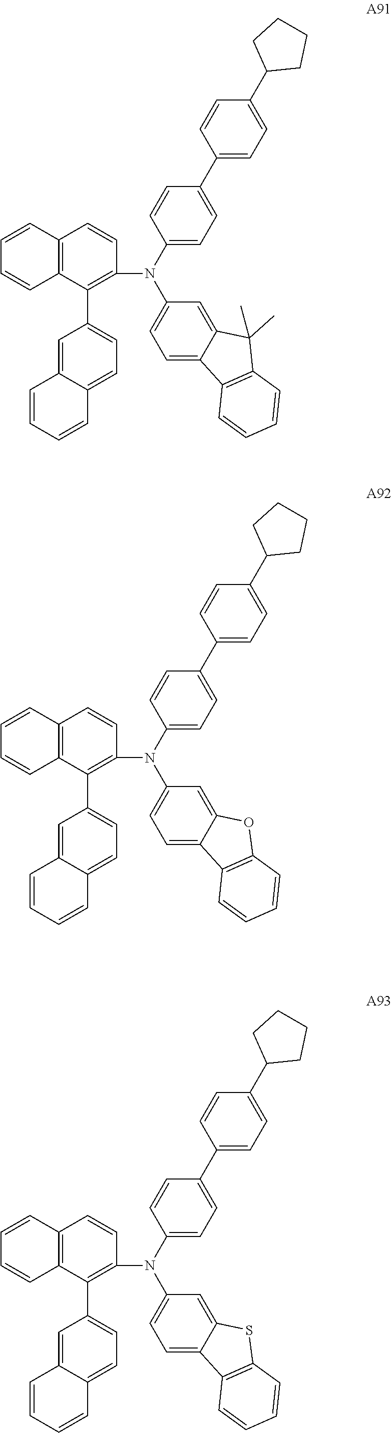 [SMILES: CC1(C)c2ccccc2-c2ccc(N(c3ccc(-c4ccc(C5CCCC5)cc4)cc3)c3ccc4ccccc4c3-c3ccc4ccccc4c3)cc21 ; c1ccc2cc(-c3c(N(c4ccc(-c5ccc(C6CCCC6)cc5)cc4)c4ccc5c(c4)oc4ccccc45)ccc4ccccc34)ccc2c1 ; c1ccc2cc(-c3c(N(c4ccc(-c5ccc(C6CCCC6)cc5)cc4)c4ccc5c(c4)sc4ccccc45)ccc4ccccc34)ccc2c1]

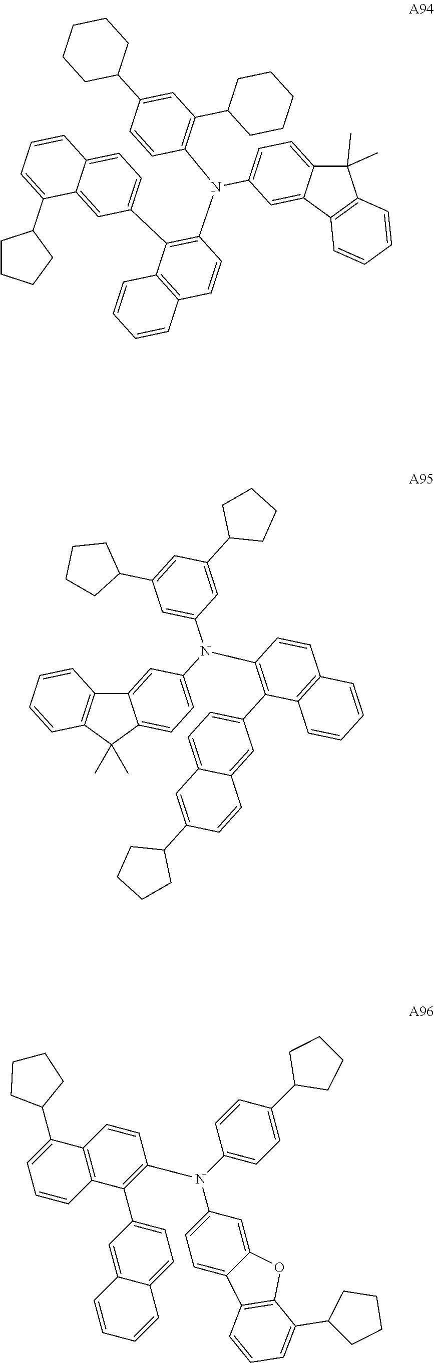 [SMILES: CC1(C)c2ccccc2-c2cc(N(c3ccc(C4CCCCC4)cc3C3CCCCC3)c3ccc4ccccc4c3-c3ccc4cccc(C5CCCC5)c4c3)ccc21 ; CC1(C)c2ccccc2-c2cc(N(c3cc(C4CCCC4)cc(C4CCCC4)c3)c3ccc4ccccc4c3-c3ccc4cc(C5CCCC5)ccc4c3)ccc21 ; c1ccc2cc(-c3c(N(c4ccc(C5CCCC5)cc4)c4ccc5c(c4)oc4c(C6CCCC6)cccc45)ccc4c(C5CCCC5)cccc34)ccc2c1]

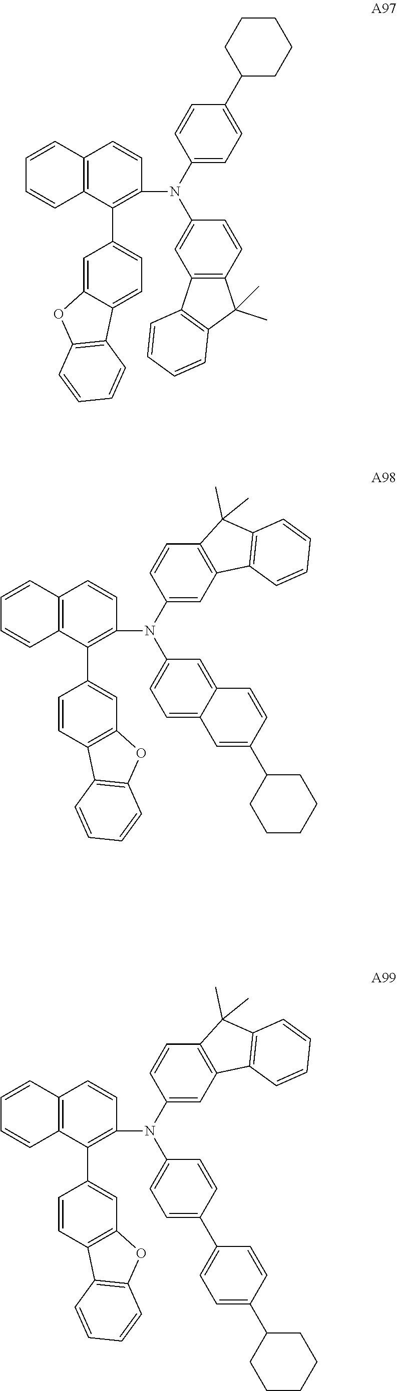 [SMILES: CC1(C)c2ccccc2-c2cc(N(c3ccc(C4CCCCC4)cc3)c3ccc4ccccc4c3-c3ccc4c(c3)oc3ccccc34)ccc21 ; CC1(C)c2ccccc2-c2cc(N(c3ccc4cc(C5CCCCC5)ccc4c3)c3ccc4ccccc4c3-c3ccc4c(c3)oc3ccccc34)ccc21 ; CC1(C)c2ccccc2-c2cc(N(c3ccc(-c4ccc(C5CCCCC5)cc4)cc3)c3ccc4ccccc4c3-c3ccc4c(c3)oc3ccccc34)ccc21]

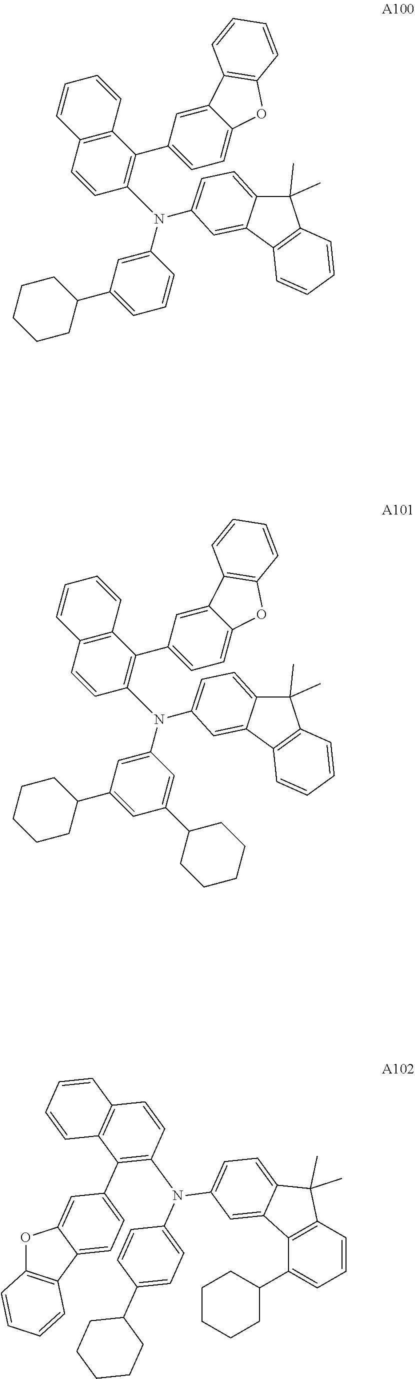 [SMILES: CC1(C)c2ccccc2-c2cc(N(c3cccc(C4CCCCC4)c3)c3ccc4ccccc4c3-c3ccc4oc5ccccc5c4c3)ccc21 ; CC1(C)c2ccccc2-c2cc(N(c3cc(C4CCCCC4)cc(C4CCCCC4)c3)c3ccc4ccccc4c3-c3ccc4oc5ccccc5c4c3)ccc21 ; CC1(C)c2ccc(N(c3ccc(C4CCCCC4)cc3)c3ccc4ccccc4c3-c3ccc4c(c3)oc3ccccc34)cc2-c2c(C3CCCCC3)cccc21]

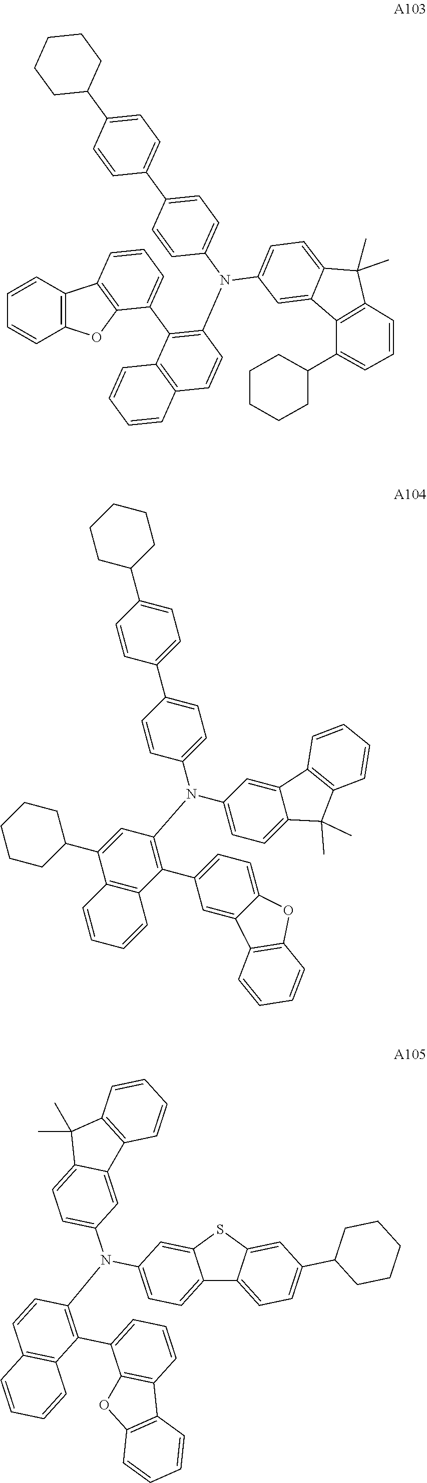 [SMILES: CC1(C)c2ccc(N(c3ccc(-c4ccc(C5CCCCC5)cc4)cc3)c3ccc4ccccc4c3-c3cccc4c3oc3ccccc34)cc2-c2c(C3CCCCC3)cccc21 ; CC1(C)c2ccccc2-c2cc(N(c3ccc(-c4ccc(C5CCCCC5)cc4)cc3)c3cc(C4CCCCC4)c4ccccc4c3-c3ccc4oc5ccccc5c4c3)ccc21 ; CC1(C)c2ccccc2-c2cc(N(c3ccc4c(c3)sc3cc(C5CCCCC5)ccc34)c3ccc4ccccc4c3-c3cccc4c3oc3ccccc34)ccc21]

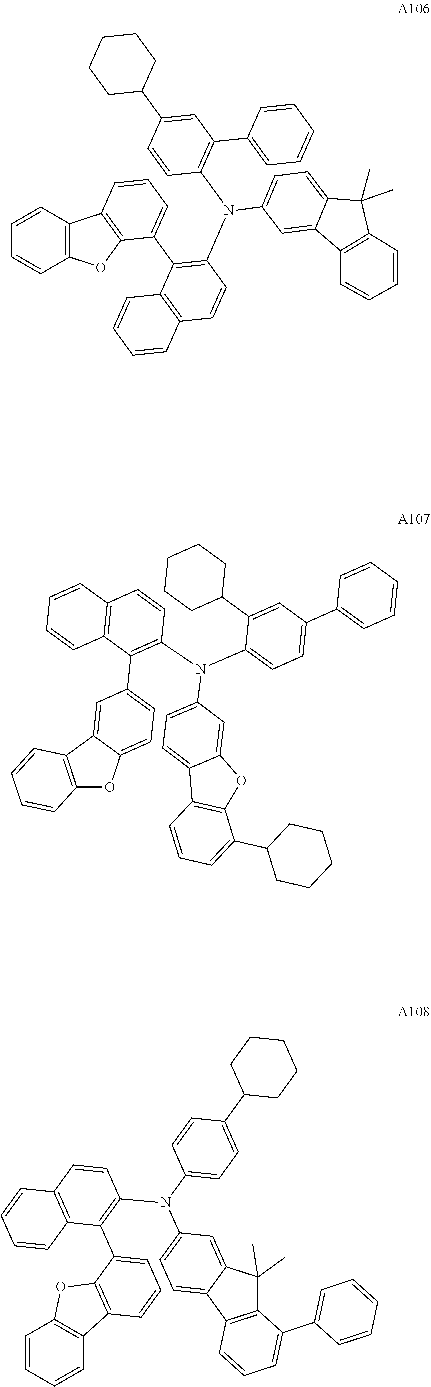 [SMILES: CC1(C)c2ccccc2-c2cc(N(c3ccc(C4CCCCC4)cc3-c3ccccc3)c3ccc4ccccc4c3-c3cccc4c3oc3ccccc34)ccc21 ; c1ccc(-c2ccc(N(c3ccc4c(c3)oc3c(C5CCCCC5)cccc34)c3ccc4ccccc4c3-c3ccc4oc5ccccc5c4c3)c(C3CCCCC3)c2)cc1 ; CC1(C)c2cc(N(c3ccc(C4CCCCC4)cc3)c3ccc4ccccc4c3-c3cccc4c3oc3ccccc34)ccc2-c2cccc(-c3ccccc3)c21]

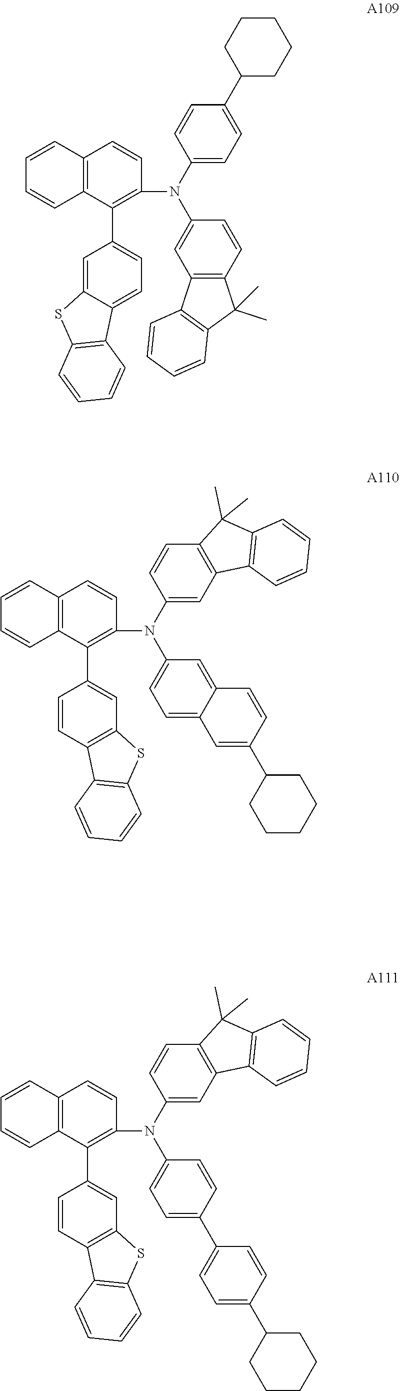 [SMILES: CC1(C)c2ccccc2-c2cc(N(c3ccc(C4CCCCC4)cc3)c3ccc4ccccc4c3-c3ccc4c(c3)sc3ccccc34)ccc21 ; CC1(C)c2ccccc2-c2cc(N(c3ccc4cc(C5CCCCC5)ccc4c3)c3ccc4ccccc4c3-c3ccc4c(c3)sc3ccccc34)ccc21 ; CC1(C)c2ccccc2-c2cc(N(c3ccc(-c4ccc(C5CCCCC5)cc4)cc3)c3ccc4ccccc4c3-c3ccc4c(c3)sc3ccccc34)ccc21]

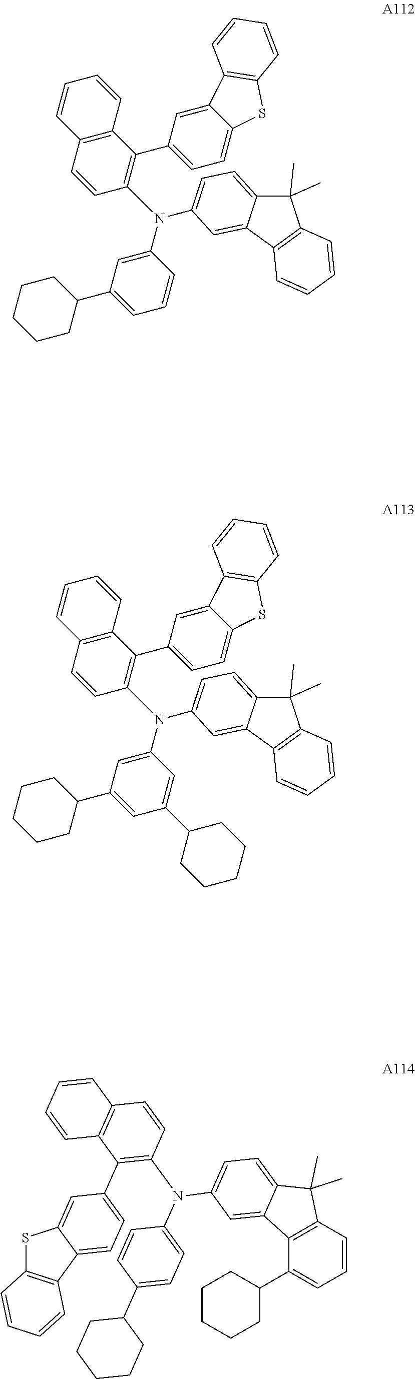 [SMILES: CC1(C)c2ccccc2-c2cc(N(c3cccc(C4CCCCC4)c3)c3ccc4ccccc4c3-c3ccc4sc5ccccc5c4c3)ccc21 ; CC1(C)c2ccccc2-c2cc(N(c3cc(C4CCCCC4)cc(C4CCCCC4)c3)c3ccc4ccccc4c3-c3ccc4sc5ccccc5c4c3)ccc21 ; CC1(C)c2ccc(N(c3ccc(C4CCCCC4)cc3)c3ccc4ccccc4c3-c3ccc4c(c3)sc3ccccc34)cc2-c2c(C3CCCCC3)cccc21]

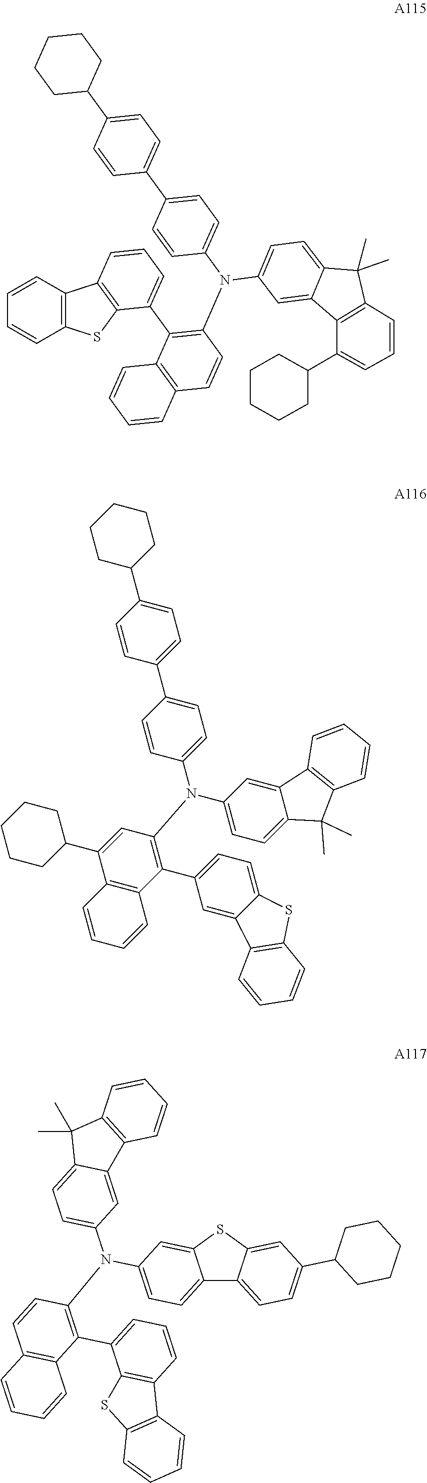 [SMILES: CC1(C)c2ccc(N(c3ccc(-c4ccc(C5CCCCC5)cc4)cc3)c3ccc4ccccc4c3-c3cccc4c3sc3ccccc34)cc2-c2c(C3CCCCC3)cccc21 ; CC1(C)c2ccccc2-c2cc(N(c3ccc(-c4ccc(C5CCCCC5)cc4)cc3)c3cc(C4CCCCC4)c4ccccc4c3-c3ccc4sc5ccccc5c4c3)ccc21 ; CC1(C)c2ccccc2-c2cc(N(c3ccc4c(c3)sc3cc(C5CCCCC5)ccc34)c3ccc4ccccc4c3-c3cccc4c3sc3ccccc34)ccc21]

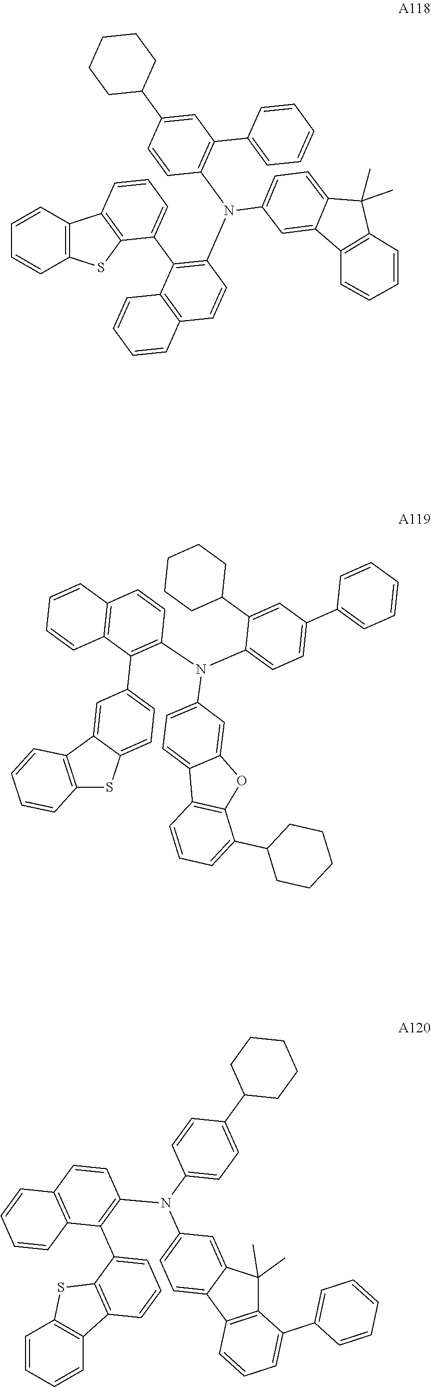 [SMILES: CC1(C)c2ccccc2-c2cc(N(c3ccc(C4CCCCC4)cc3-c3ccccc3)c3ccc4ccccc4c3-c3cccc4c3sc3ccccc34)ccc21 ; c1ccc(-c2ccc(N(c3ccc4c(c3)oc3c(C5CCCCC5)cccc34)c3ccc4ccccc4c3-c3ccc4sc5ccccc5c4c3)c(C3CCCCC3)c2)cc1 ; CC1(C)c2cc(N(c3ccc(C4CCCCC4)cc3)c3ccc4ccccc4c3-c3cccc4c3sc3ccccc34)ccc2-c2cccc(-c3ccccc3)c21]

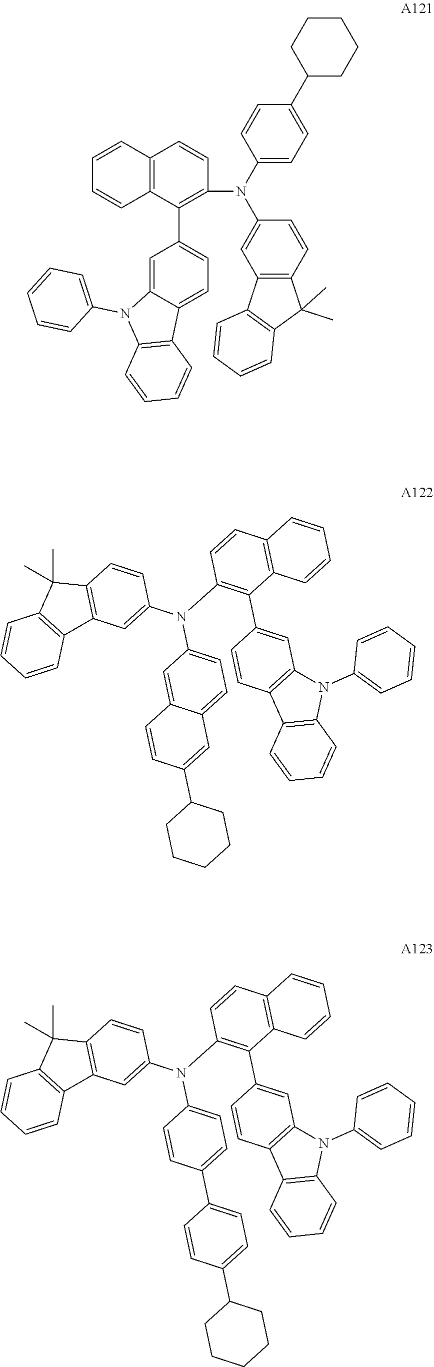 [SMILES: CC1(C)c2ccccc2-c2cc(N(c3ccc(C4CCCCC4)cc3)c3ccc4ccccc4c3-c3ccc4c5ccccc5n(-c5ccccc5)c4c3)ccc21 ; CC1(C)c2ccccc2-c2cc(N(c3ccc4cc(C5CCCCC5)ccc4c3)c3ccc4ccccc4c3-c3ccc4c5ccccc5n(-c5ccccc5)c4c3)ccc21 ; CC1(C)c2ccccc2-c2cc(N(c3ccc(-c4ccc(C5CCCCC5)cc4)cc3)c3ccc4ccccc4c3-c3ccc4c5ccccc5n(-c5ccccc5)c4c3)ccc21]

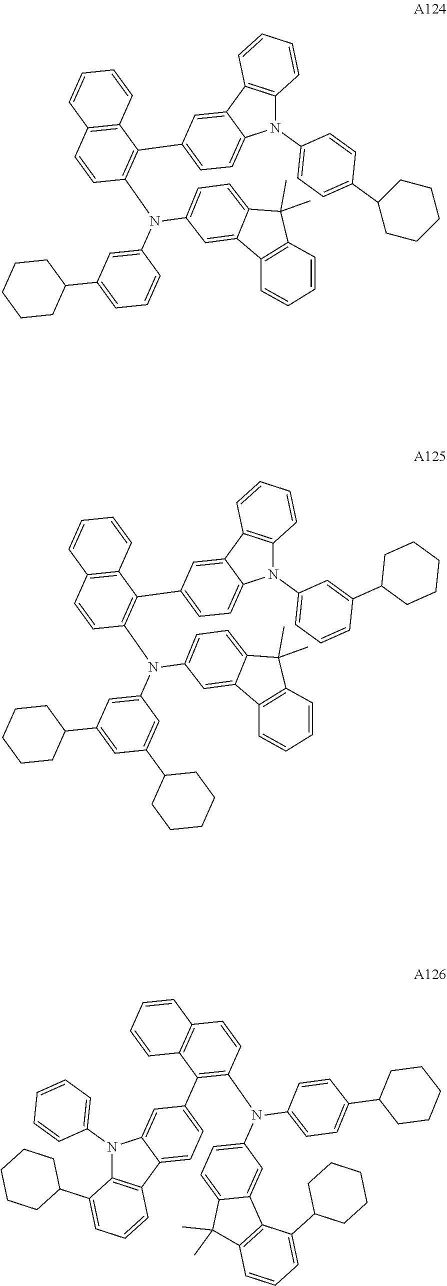 [SMILES: CC1(C)c2ccccc2-c2cc(N(c3cccc(C4CCCCC4)c3)c3ccc4ccccc4c3-c3ccc4c(c3)c3ccccc3n4-c3ccc(C4CCCCC4)cc3)ccc21 ; CC1(C)c2ccccc2-c2cc(N(c3cc(C4CCCCC4)cc(C4CCCCC4)c3)c3ccc4ccccc4c3-c3ccc4c(c3)c3ccccc3n4-c3cccc(C4CCCCC4)c3)ccc21 ; CC1(C)c2ccc(N(c3ccc(C4CCCCC4)cc3)c3ccc4ccccc4c3-c3ccc4c5cccc(C6CCCCC6)c5n(-c5ccccc5)c4c3)cc2-c2c(C3CCCCC3)cccc21]

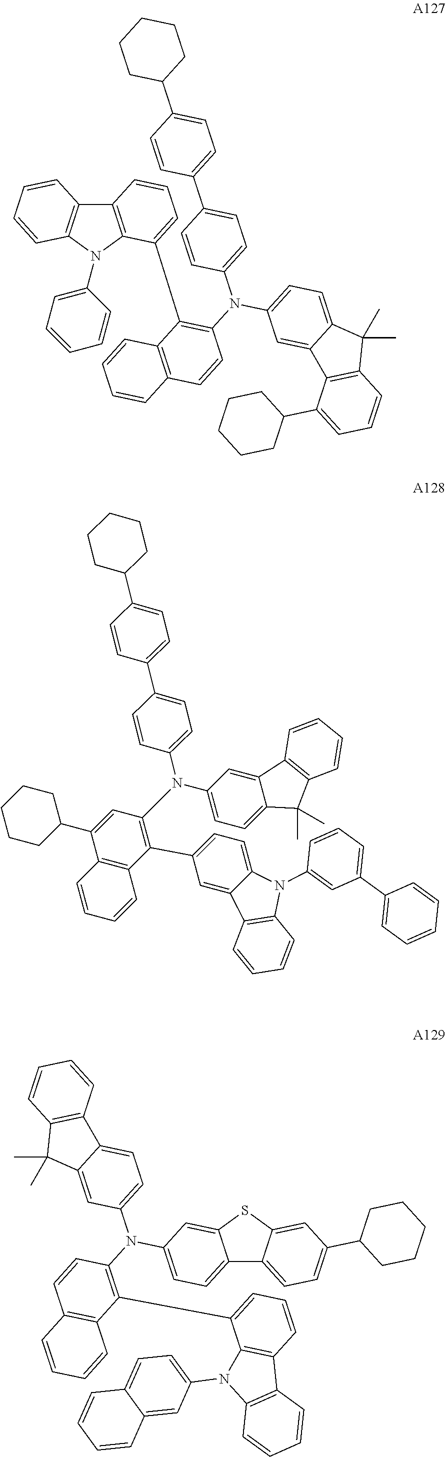

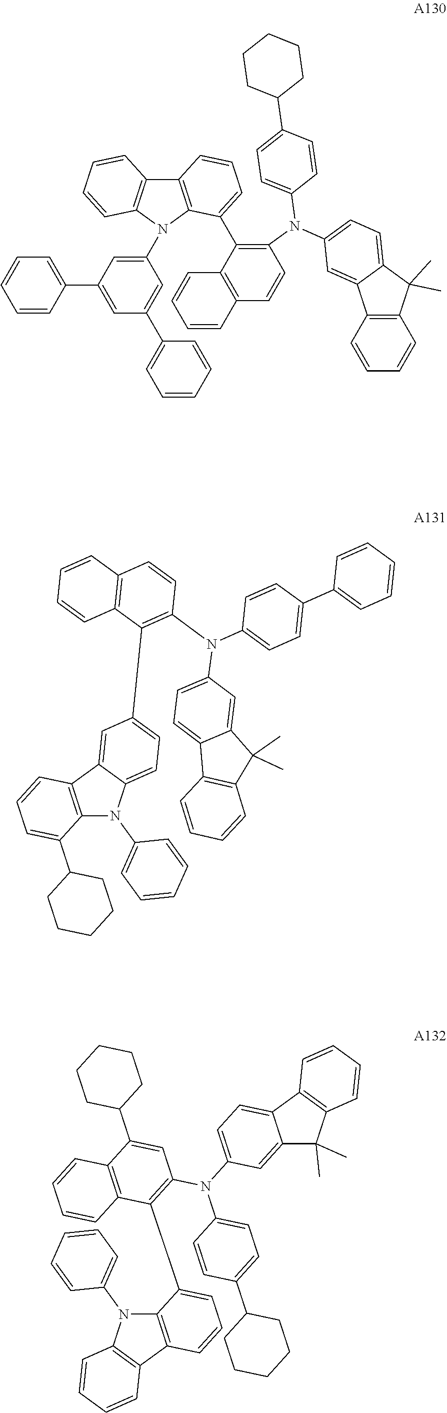 [SMILES: CC1(C)c2ccccc2-c2cc(N(c3ccc(C4CCCCC4)cc3)c3ccc4ccccc4c3-c3cccc4c5ccccc5n(-c5cc(-c6ccccc6)cc(-c6ccccc6)c5)c34)ccc21 ; CC1(C)c2ccccc2-c2ccc(N(c3ccc(-c4ccccc4)cc3)c3ccc4ccccc4c3-c3ccc4c(c3)c3cccc(C5CCCCC5)c3n4-c3ccccc3)cc21 ; CC1(C)c2ccccc2-c2ccc(N(c3ccc(C4CCCCC4)cc3)c3cc(C4CCCCC4)c4ccccc4c3-c3cccc4c5ccccc5n(-c5ccccc5)c34)cc21]

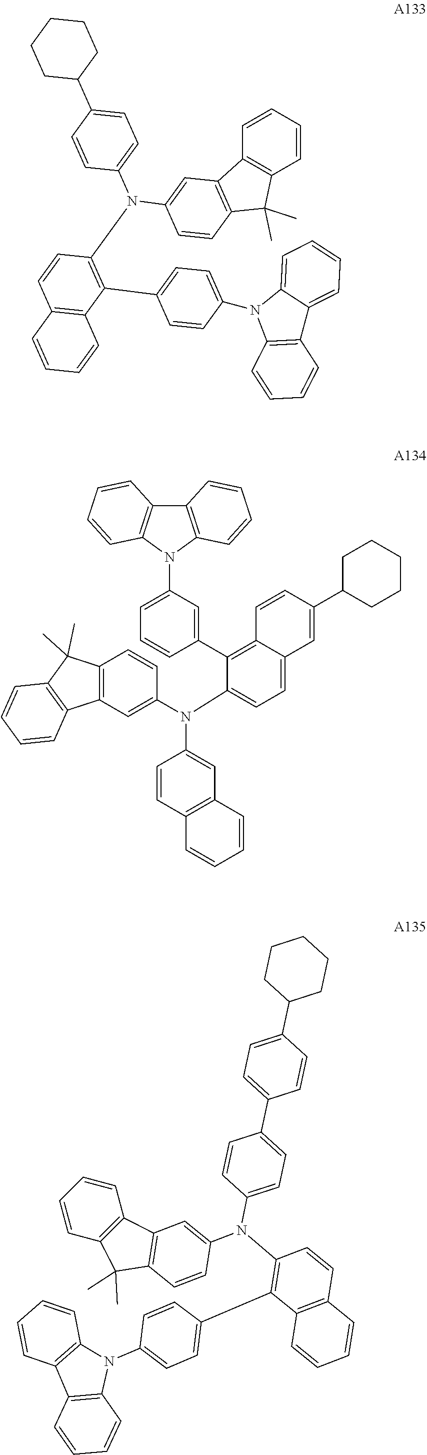 [SMILES: CC1(C)c2ccccc2-c2cc(N(c3ccc(C4CCCCC4)cc3)c3ccc4ccccc4c3-c3ccc(-n4c5ccccc5c5ccccc54)cc3)ccc21 ; CC1(C)c2ccccc2-c2cc(N(c3ccc4ccccc4c3)c3ccc4cc(C5CCCCC5)ccc4c3-c3cccc(-n4c5ccccc5c5ccccc54)c3)ccc21 ; CC1(C)c2ccccc2-c2cc(N(c3ccc(-c4ccc(C5CCCCC5)cc4)cc3)c3ccc4ccccc4c3-c3ccc(-n4c5ccccc5c5ccccc54)cc3)ccc21]

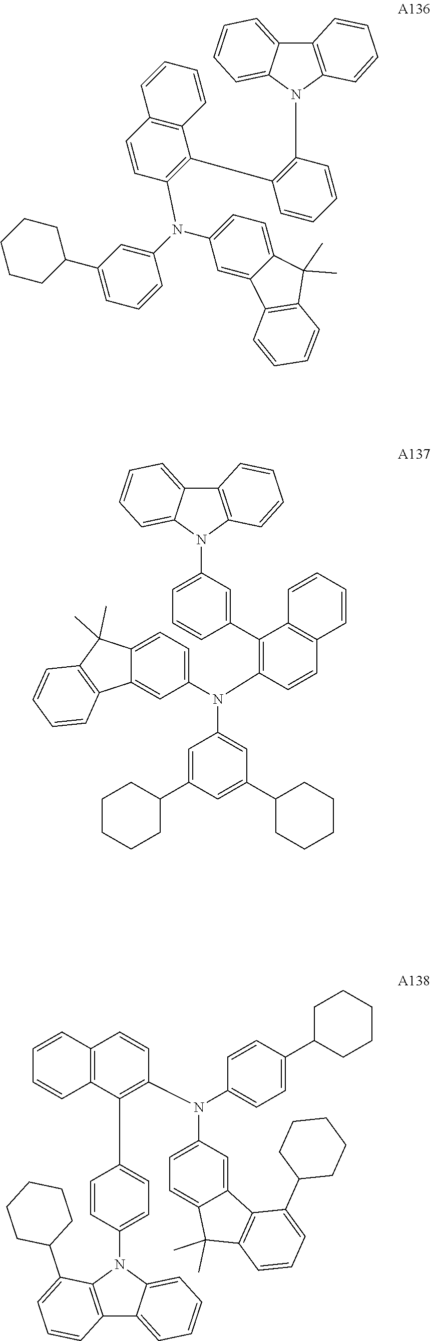 [SMILES: CC1(C)c2ccccc2-c2cc(N(c3cccc(C4CCCCC4)c3)c3ccc4ccccc4c3-c3ccccc3-n3c4ccccc4c4ccccc43)ccc21 ; CC1(C)c2ccccc2-c2cc(N(c3cc(C4CCCCC4)cc(C4CCCCC4)c3)c3ccc4ccccc4c3-c3cccc(-n4c5ccccc5c5ccccc54)c3)ccc21 ; CC1(C)c2ccc(N(c3ccc(C4CCCCC4)cc3)c3ccc4ccccc4c3-c3ccc(-n4c5ccccc5c5cccc(C6CCCCC6)c54)cc3)cc2-c2c(C3CCCCC3)cccc21]

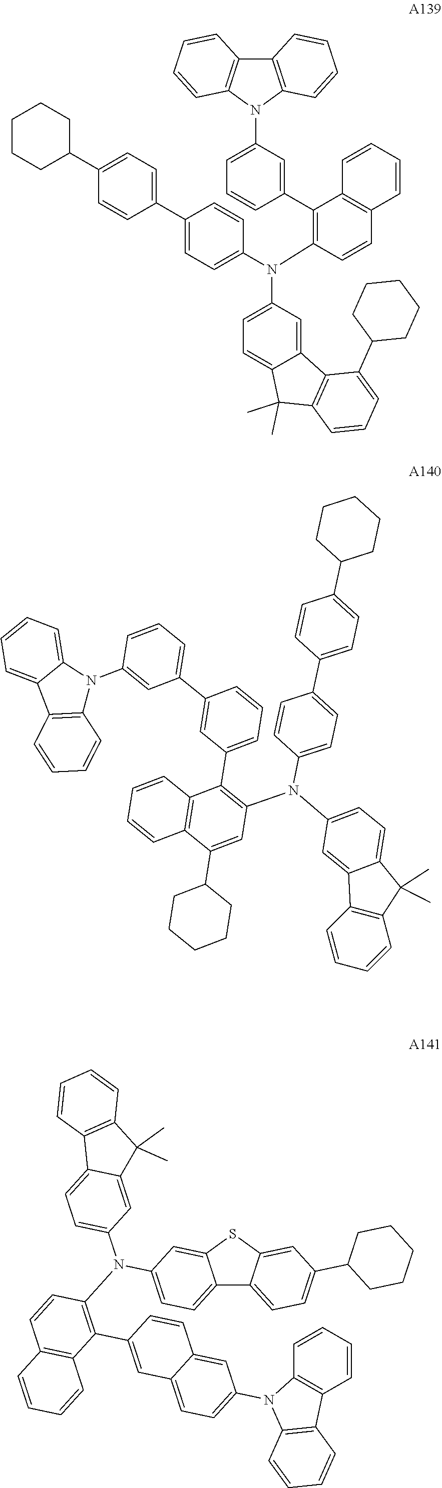 [SMILES: CC1(C)c2ccc(N(c3ccc(-c4ccc(C5CCCCC5)cc4)cc3)c3ccc4ccccc4c3-c3cccc(-n4c5ccccc5c5ccccc54)c3)cc2-c2c(C3CCCCC3)cccc21 ; CC1(C)c2ccccc2-c2cc(N(c3ccc(-c4ccc(C5CCCCC5)cc4)cc3)c3cc(C4CCCCC4)c4ccccc4c3-c3cccc(-c4cccc(-n5c6ccccc6c6ccccc65)c4)c3)ccc21 ; CC1(C)c2ccccc2-c2ccc(N(c3ccc4c(c3)sc3cc(C5CCCCC5)ccc34)c3ccc4ccccc4c3-c3ccc4cc(-n5c6ccccc6c6ccccc65)ccc4c3)cc21]

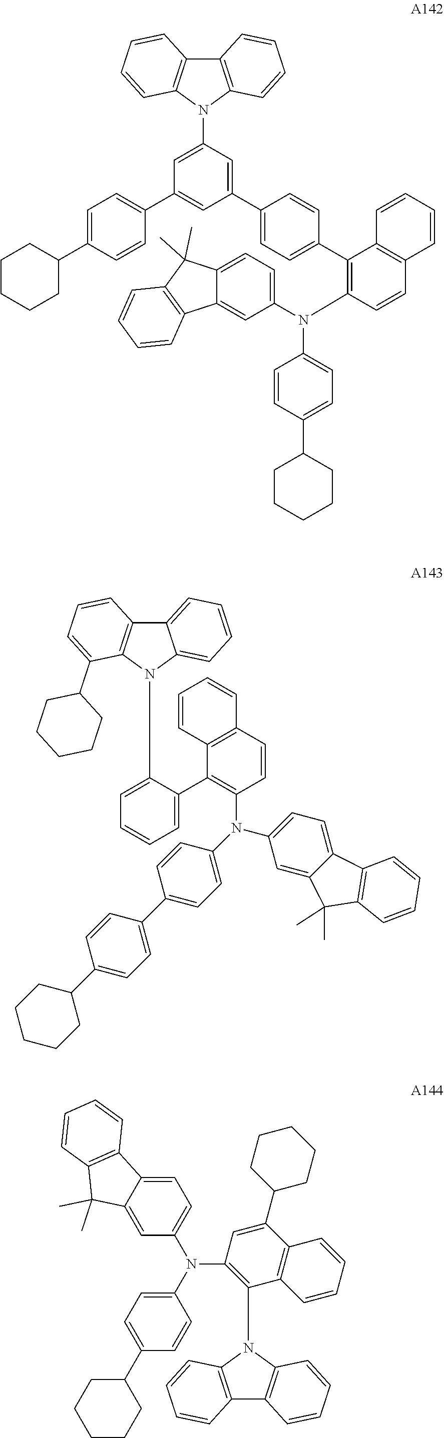 [SMILES: CC1(C)c2ccccc2-c2cc(N(c3ccc(C4CCCCC4)cc3)c3ccc4ccccc4c3-c3ccc(-c4cc(-c5ccc(C6CCCCC6)cc5)cc(-n5c6ccccc6c6ccccc65)c4)cc3)ccc21 ; CC1(C)c2ccccc2-c2ccc(N(c3ccc(-c4ccc(C5CCCCC5)cc4)cc3)c3ccc4ccccc4c3-c3ccccc3-n3c4ccccc4c4cccc(C5CCCCC5)c43)cc21 ; CC1(C)c2ccccc2-c2ccc(N(c3ccc(C4CCCCC4)cc3)c3cc(C4CCCCC4)c4ccccc4c3-n3c4ccccc4c4ccccc43)cc21]

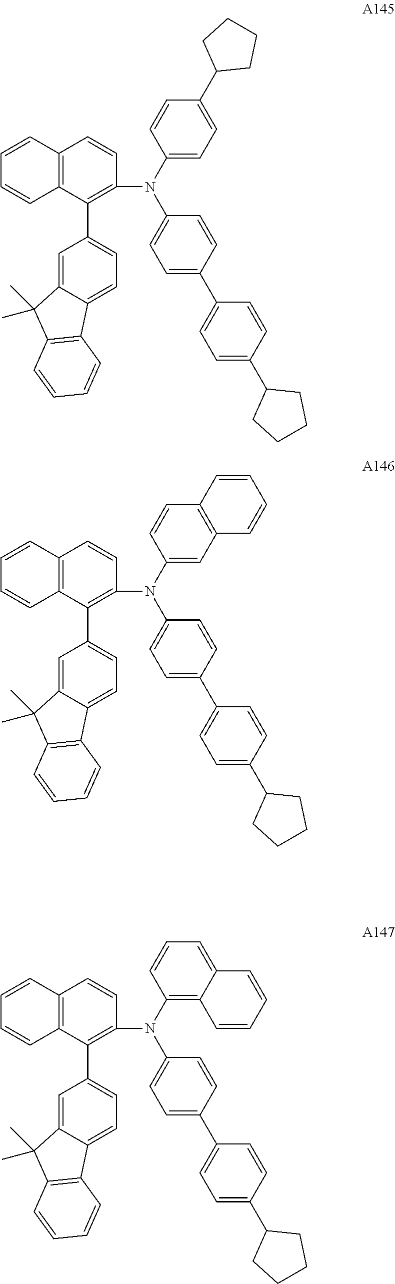

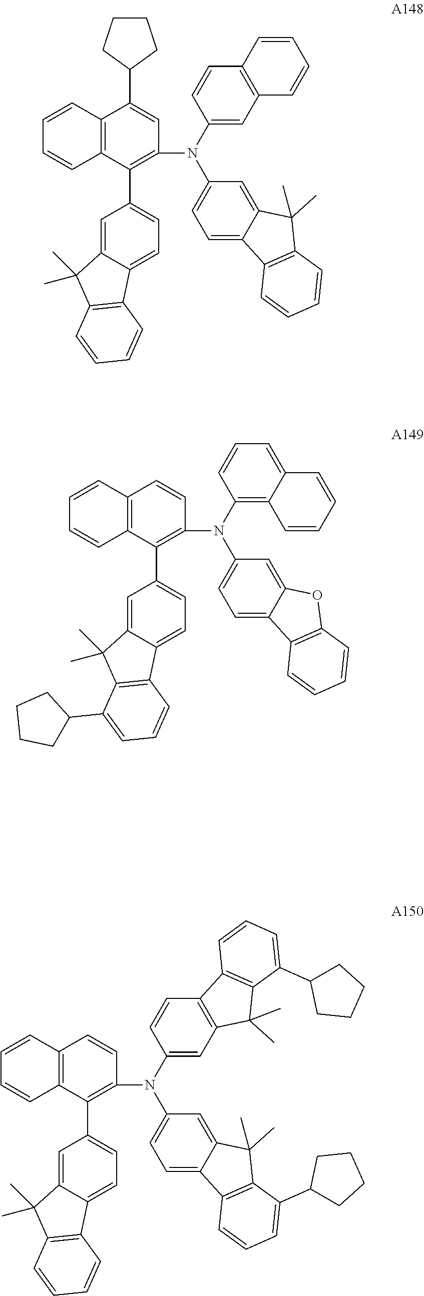 [SMILES: CC1(C)c2ccccc2-c2ccc(-c3c(N(c4ccc5c(c4)C(C)(C)c4ccccc4-5)c4ccc5ccccc5c4)cc(C4CCCC4)c4ccccc34)cc21 ; CC1(C)c2cc(-c3c(N(c4ccc5c(c4)oc4ccccc45)c4cccc5ccccc45)ccc4ccccc34)ccc2-c2cccc(C3CCCC3)c21 ; CC1(C)c2ccccc2-c2ccc(-c3c(N(c4ccc5c(c4)C(C)(C)c4c-5cccc4C4CCCC4)c4ccc5c(c4)C(C)(C)c4c-5cccc4C4CCCC4)ccc4ccccc34)cc21]

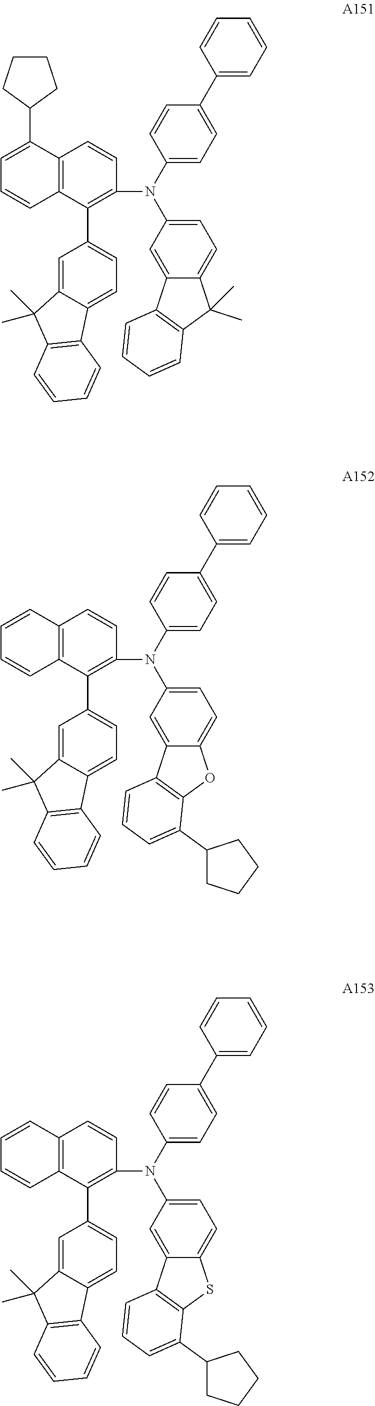 [SMILES: CC1(C)c2ccccc2-c2cc(N(c3ccc(-c4ccccc4)cc3)c3ccc4c(C5CCCC5)cccc4c3-c3ccc4c(c3)C(C)(C)c3ccccc3-4)ccc21 ; CC1(C)c2ccccc2-c2ccc(-c3c(N(c4ccc(-c5ccccc5)cc4)c4ccc5oc6c(C7CCCC7)cccc6c5c4)ccc4ccccc34)cc21 ; CC1(C)c2ccccc2-c2ccc(-c3c(N(c4ccc(-c5ccccc5)cc4)c4ccc5sc6c(C7CCCC7)cccc6c5c4)ccc4ccccc34)cc21]

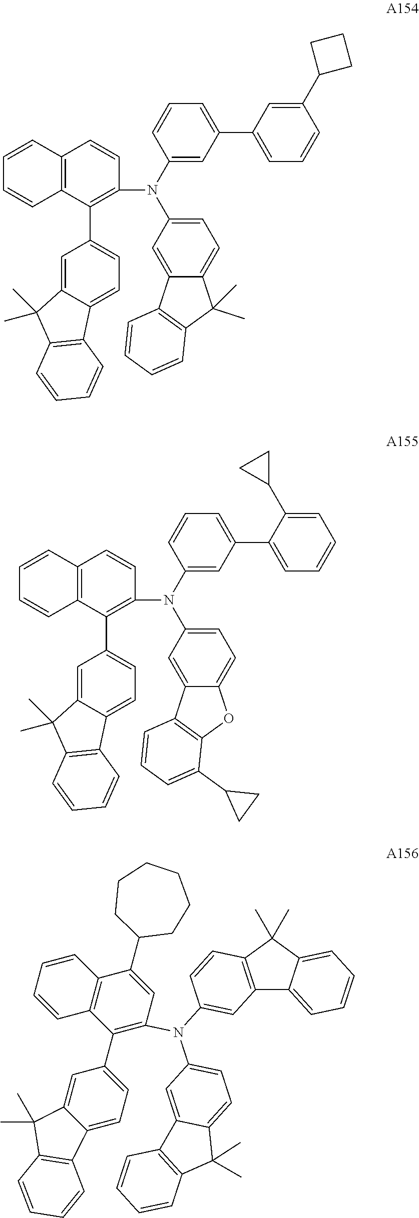 [SMILES: CC1(C)c2ccccc2-c2cc(N(c3cccc(-c4cccc(C5CCC5)c4)c3)c3ccc4ccccc4c3-c3ccc4c(c3)C(C)(C)c3ccccc3-4)ccc21 ; CC1(C)c2ccccc2-c2ccc(-c3c(N(c4cccc(-c5ccccc5C5CC5)c4)c4ccc5oc6c(C7CC7)cccc6c5c4)ccc4ccccc34)cc21 ; CC1(C)c2ccccc2-c2cc(N(c3ccc4c(c3)-c3ccccc3C4(C)C)c3cc(C4CCCCCC4)c4ccccc4c3-c3ccc4c(c3)C(C)(C)c3ccccc3-4)ccc21]

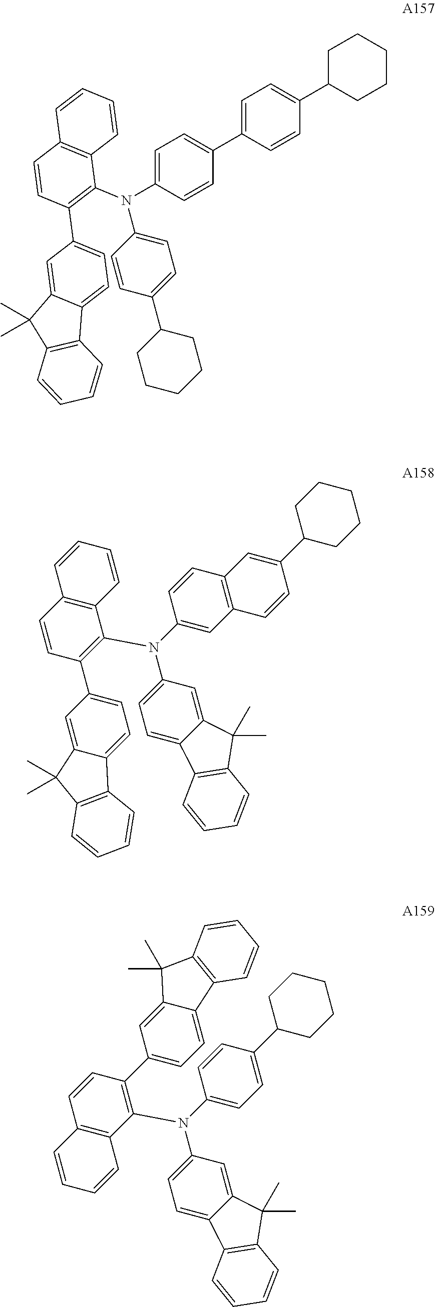 [SMILES: CC1(C)c2ccccc2-c2ccc(-c3ccc4ccccc4c3N(c3ccc(-c4ccc(C5CCCCC5)cc4)cc3)c3ccc(C4CCCCC4)cc3)cc21 ; CC1(C)c2ccccc2-c2ccc(-c3ccc4ccccc4c3N(c3ccc4c(c3)C(C)(C)c3ccccc3-4)c3ccc4cc(C5CCCCC5)ccc4c3)cc21 ; CC1(C)c2ccccc2-c2ccc(-c3ccc4ccccc4c3N(c3ccc(C4CCCCC4)cc3)c3ccc4c(c3)C(C)(C)c3ccccc3-4)cc21]

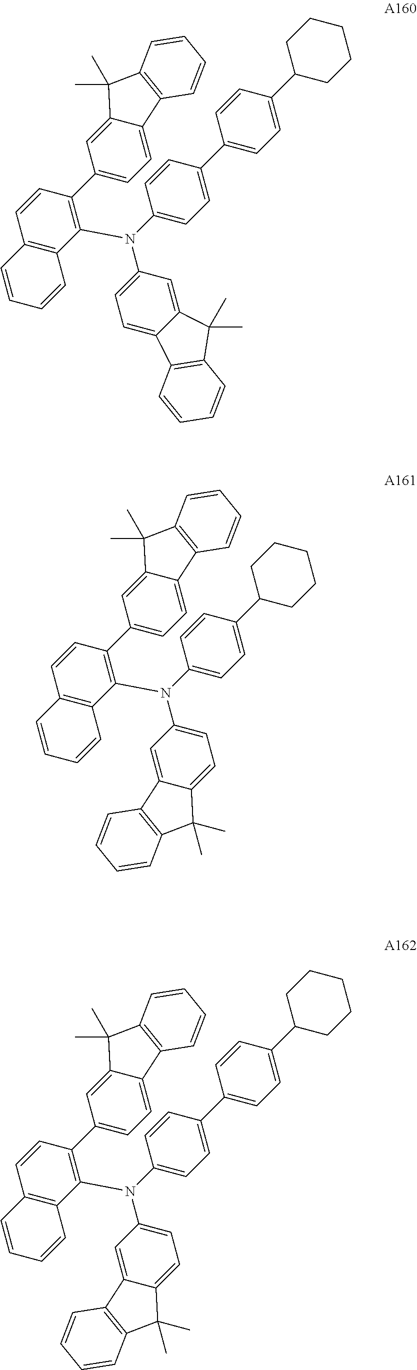 [SMILES: CC1(C)c2ccccc2-c2ccc(-c3ccc4ccccc4c3N(c3ccc(-c4ccc(C5CCCCC5)cc4)cc3)c3ccc4c(c3)C(C)(C)c3ccccc3-4)cc21 ; CC1(C)c2ccccc2-c2cc(N(c3ccc(C4CCCCC4)cc3)c3c(-c4ccc5c(c4)C(C)(C)c4ccccc4-5)ccc4ccccc34)ccc21 ; CC1(C)c2ccccc2-c2cc(N(c3ccc(-c4ccc(C5CCCCC5)cc4)cc3)c3c(-c4ccc5c(c4)C(C)(C)c4ccccc4-5)ccc4ccccc34)ccc21]

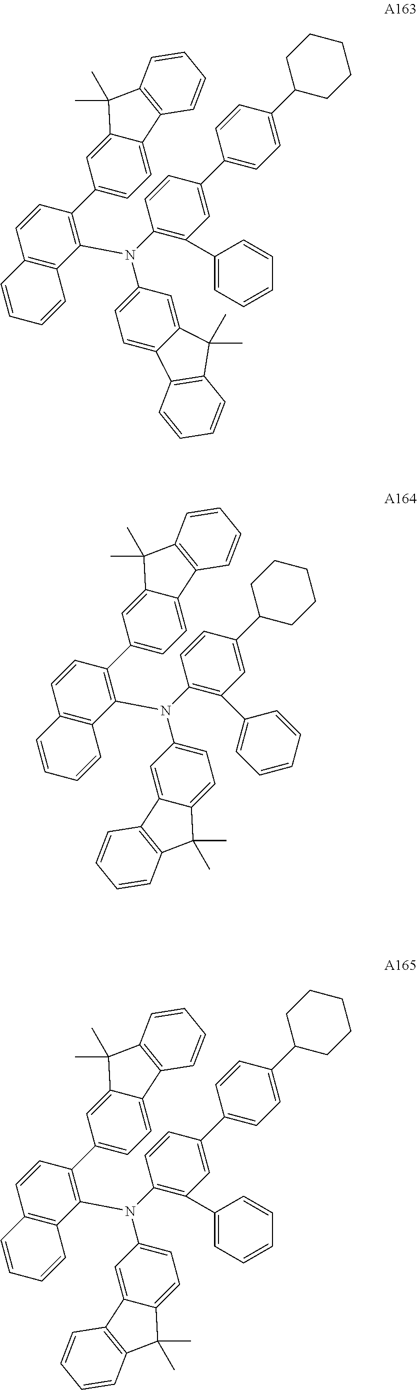 [SMILES: CC1(C)c2ccccc2-c2ccc(-c3ccc4ccccc4c3N(c3ccc4c(c3)C(C)(C)c3ccccc3-4)c3ccc(-c4ccc(C5CCCCC5)cc4)cc3-c3ccccc3)cc21 ; CC1(C)c2ccccc2-c2cc(N(c3ccc(C4CCCCC4)cc3-c3ccccc3)c3c(-c4ccc5c(c4)C(C)(C)c4ccccc4-5)ccc4ccccc34)ccc21 ; CC1(C)c2ccccc2-c2cc(N(c3ccc(-c4ccc(C5CCCCC5)cc4)cc3-c3ccccc3)c3c(-c4ccc5c(c4)C(C)(C)c4ccccc4-5)ccc4ccccc34)ccc21]

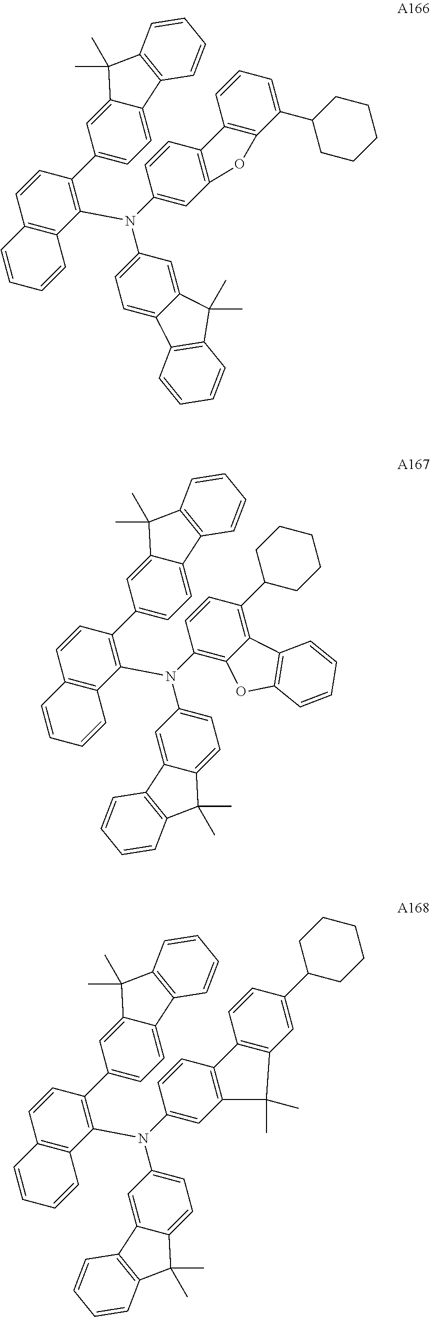 [SMILES: CC1(C)c2ccccc2-c2ccc(-c3ccc4ccccc4c3N(c3ccc4c(c3)C(C)(C)c3ccccc3-4)c3ccc4c(c3)oc3c(C5CCCCC5)cccc34)cc21 ; CC1(C)c2ccccc2-c2cc(N(c3c(-c4ccc5c(c4)C(C)(C)c4ccccc4-5)ccc4ccccc34)c3ccc(C4CCCCC4)c4c3oc3ccccc34)ccc21 ; CC1(C)c2ccccc2-c2cc(N(c3ccc4c(c3)C(C)(C)c3cc(C5CCCCC5)ccc3-4)c3c(-c4ccc5c(c4)C(C)(C)c4ccccc4-5)ccc4ccccc34)ccc21]

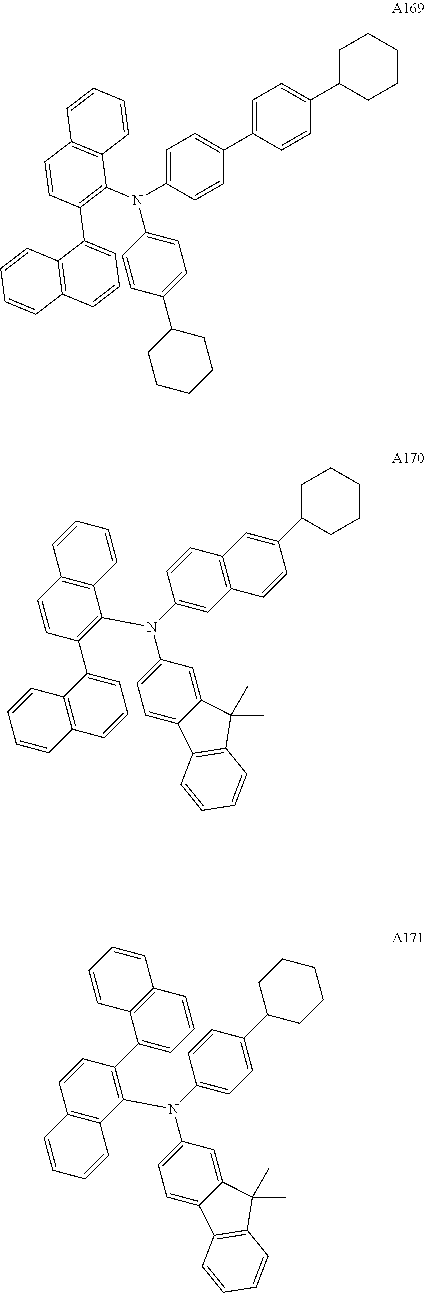 [SMILES: c1ccc2c(-c3ccc4ccccc4c3N(c3ccc(-c4ccc(C5CCCCC5)cc4)cc3)c3ccc(C4CCCCC4)cc3)cccc2c1 ; CC1(C)c2ccccc2-c2ccc(N(c3ccc4cc(C5CCCCC5)ccc4c3)c3c(-c4cccc5ccccc45)ccc4ccccc34)cc21 ; CC1(C)c2ccccc2-c2ccc(N(c3ccc(C4CCCCC4)cc3)c3c(-c4cccc5ccccc45)ccc4ccccc34)cc21]

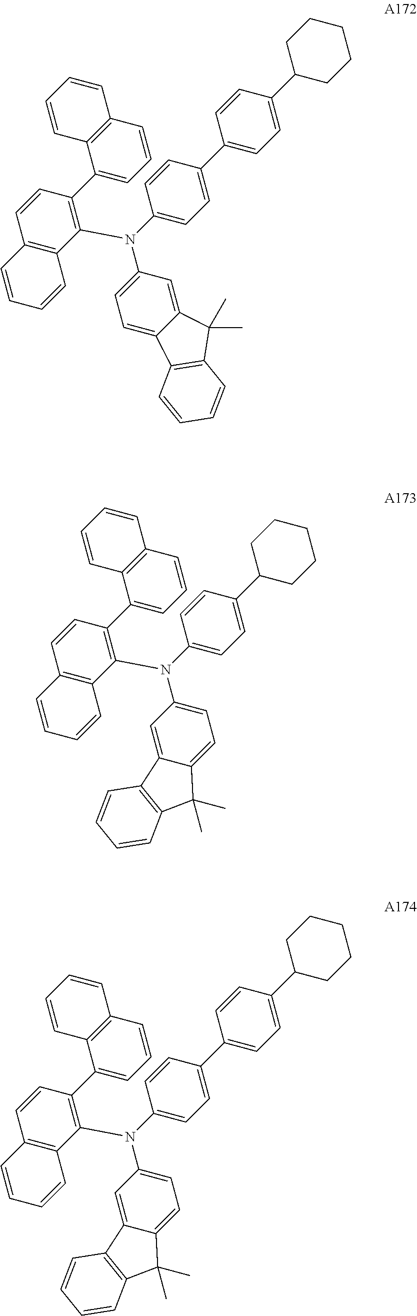 [SMILES: CC1(C)c2ccccc2-c2ccc(N(c3ccc(-c4ccc(C5CCCCC5)cc4)cc3)c3c(-c4cccc5ccccc45)ccc4ccccc34)cc21 ; CC1(C)c2ccccc2-c2cc(N(c3ccc(C4CCCCC4)cc3)c3c(-c4cccc5ccccc45)ccc4ccccc34)ccc21 ; CC1(C)c2ccccc2-c2cc(N(c3ccc(-c4ccc(C5CCCCC5)cc4)cc3)c3c(-c4cccc5ccccc45)ccc4ccccc34)ccc21]

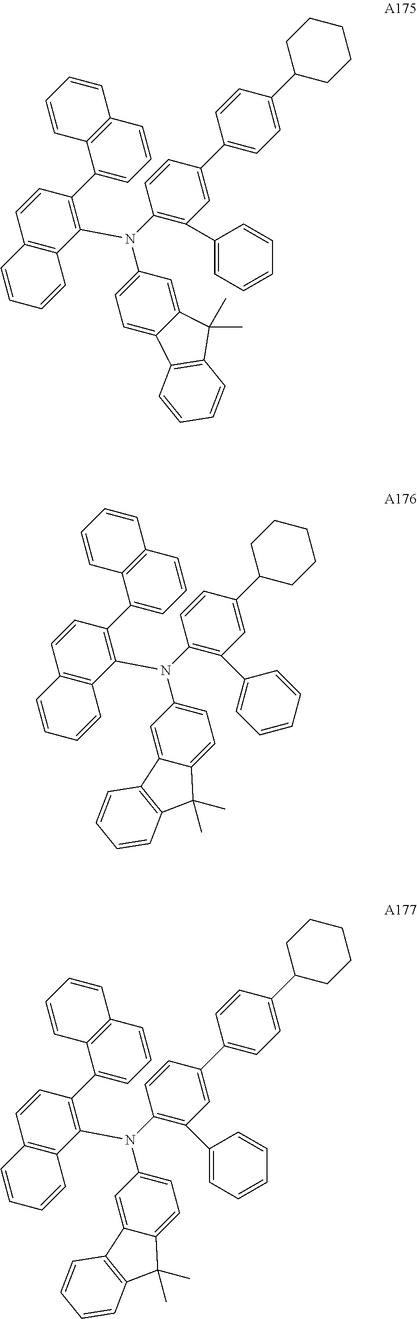 [SMILES: CC1(C)c2ccccc2-c2ccc(N(c3ccc(-c4ccc(C5CCCCC5)cc4)cc3-c3ccccc3)c3c(-c4cccc5ccccc45)ccc4ccccc34)cc21 ; CC1(C)c2ccccc2-c2cc(N(c3ccc(C4CCCCC4)cc3-c3ccccc3)c3c(-c4cccc5ccccc45)ccc4ccccc34)ccc21 ; CC1(C)c2ccccc2-c2cc(N(c3ccc(-c4ccc(C5CCCCC5)cc4)cc3-c3ccccc3)c3c(-c4cccc5ccccc45)ccc4ccccc34)ccc21]

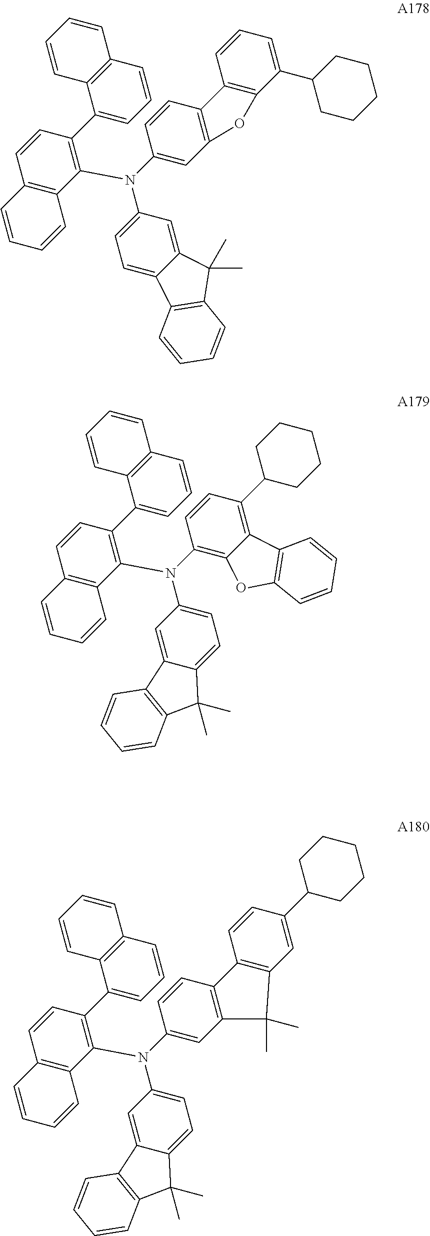 [SMILES: CC1(C)c2ccccc2-c2ccc(N(c3ccc4c(c3)oc3c(C5CCCCC5)cccc34)c3c(-c4cccc5ccccc45)ccc4ccccc34)cc21 ; CC1(C)c2ccccc2-c2cc(N(c3c(-c4cccc5ccccc45)ccc4ccccc34)c3ccc(C4CCCCC4)c4c3oc3ccccc34)ccc21 ; CC1(C)c2ccccc2-c2cc(N(c3ccc4c(c3)C(C)(C)c3cc(C5CCCCC5)ccc3-4)c3c(-c4cccc5ccccc45)ccc4ccccc34)ccc21]

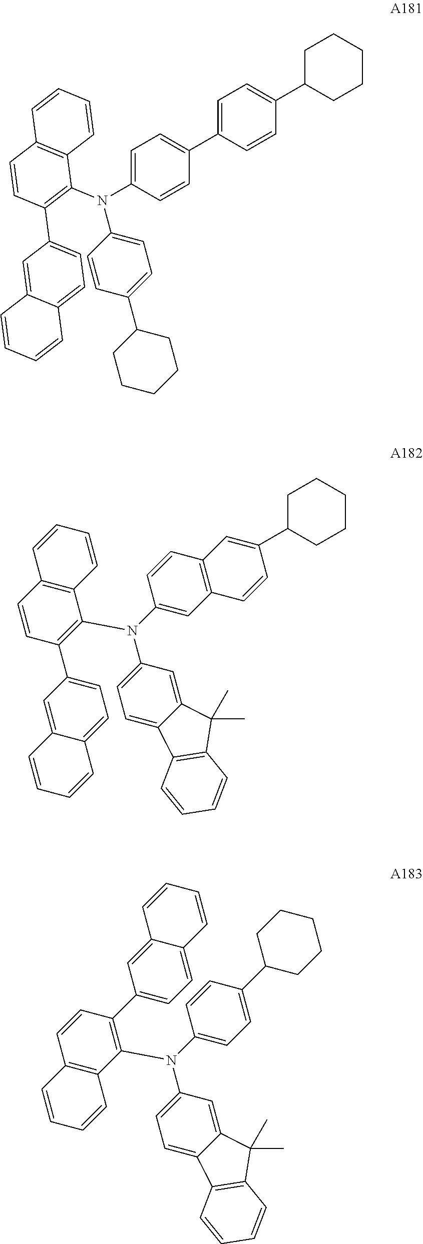 [SMILES: c1ccc2cc(-c3ccc4ccccc4c3N(c3ccc(-c4ccc(C5CCCCC5)cc4)cc3)c3ccc(C4CCCCC4)cc3)ccc2c1 ; CC1(C)c2ccccc2-c2ccc(N(c3ccc4cc(C5CCCCC5)ccc4c3)c3c(-c4ccc5ccccc5c4)ccc4ccccc34)cc21 ; CC1(C)c2ccccc2-c2ccc(N(c3ccc(C4CCCCC4)cc3)c3c(-c4ccc5ccccc5c4)ccc4ccccc34)cc21]

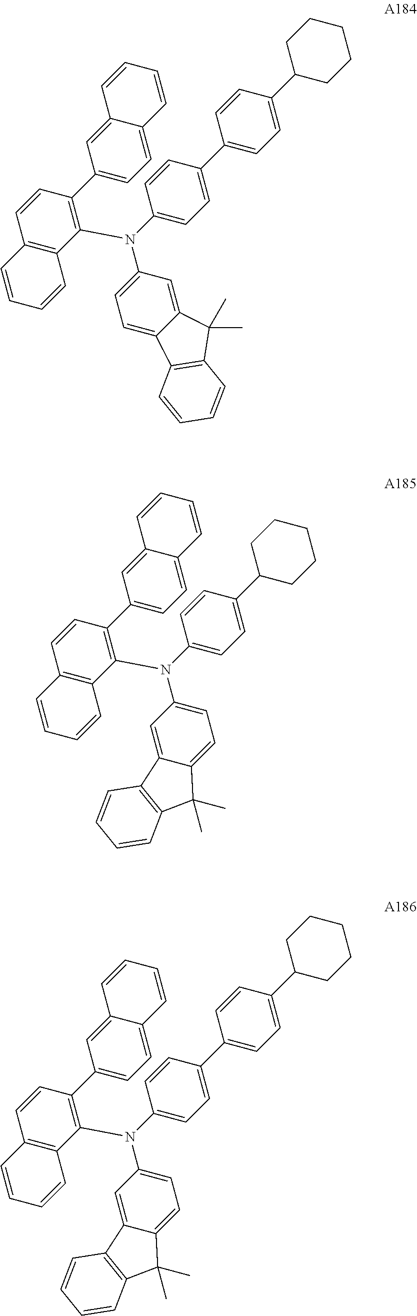 [SMILES: CC1(C)c2ccccc2-c2ccc(N(c3ccc(-c4ccc(C5CCCCC5)cc4)cc3)c3c(-c4ccc5ccccc5c4)ccc4ccccc34)cc21 ; CC1(C)c2ccccc2-c2cc(N(c3ccc(C4CCCCC4)cc3)c3c(-c4ccc5ccccc5c4)ccc4ccccc34)ccc21 ; CC1(C)c2ccccc2-c2cc(N(c3ccc(-c4ccc(C5CCCCC5)cc4)cc3)c3c(-c4ccc5ccccc5c4)ccc4ccccc34)ccc21]

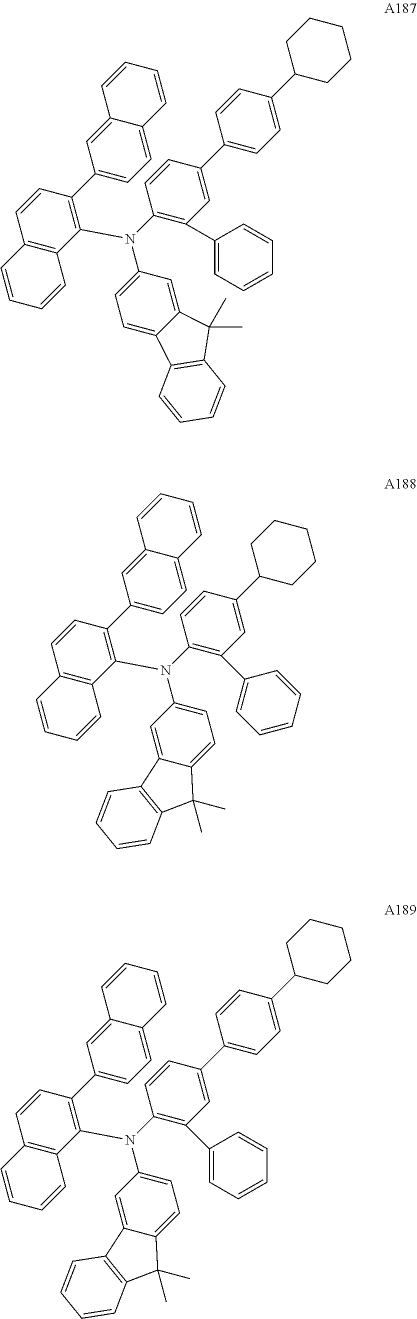 [SMILES: CC1(C)c2ccccc2-c2ccc(N(c3ccc(-c4ccc(C5CCCCC5)cc4)cc3-c3ccccc3)c3c(-c4ccc5ccccc5c4)ccc4ccccc34)cc21 ; CC1(C)c2ccccc2-c2cc(N(c3ccc(C4CCCCC4)cc3-c3ccccc3)c3c(-c4ccc5ccccc5c4)ccc4ccccc34)ccc21 ; CC1(C)c2ccccc2-c2cc(N(c3ccc(-c4ccc(C5CCCCC5)cc4)cc3-c3ccccc3)c3c(-c4ccc5ccccc5c4)ccc4ccccc34)ccc21]

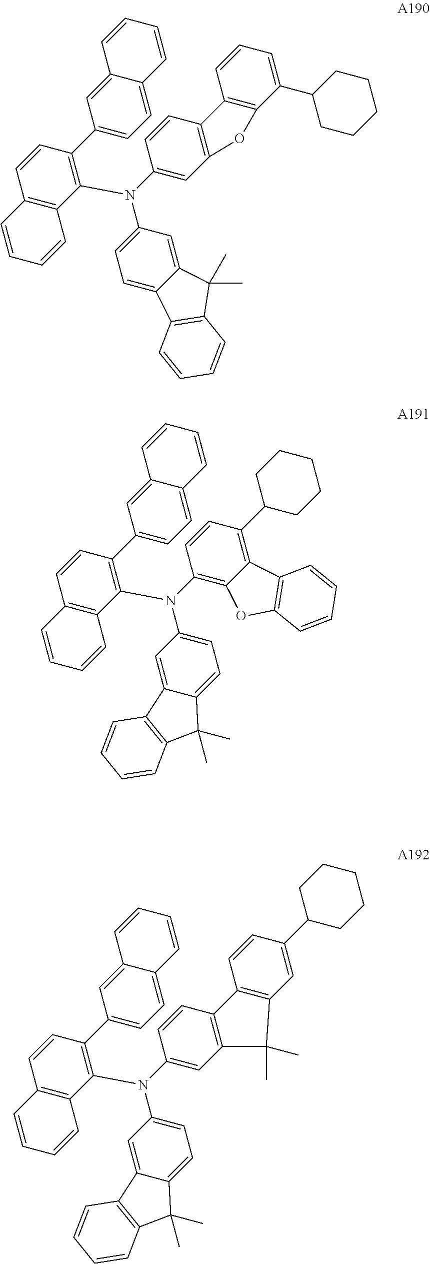 [SMILES: CC1(C)c2ccccc2-c2ccc(N(c3ccc4c(c3)oc3c(C5CCCCC5)cccc34)c3c(-c4ccc5ccccc5c4)ccc4ccccc34)cc21 ; CC1(C)c2ccccc2-c2cc(N(c3c(-c4ccc5ccccc5c4)ccc4ccccc34)c3ccc(C4CCCCC4)c4c3oc3ccccc34)ccc21 ; CC1(C)c2ccccc2-c2cc(N(c3ccc4c(c3)C(C)(C)c3cc(C5CCCCC5)ccc3-4)c3c(-c4ccc5ccccc5c4)ccc4ccccc34)ccc21]

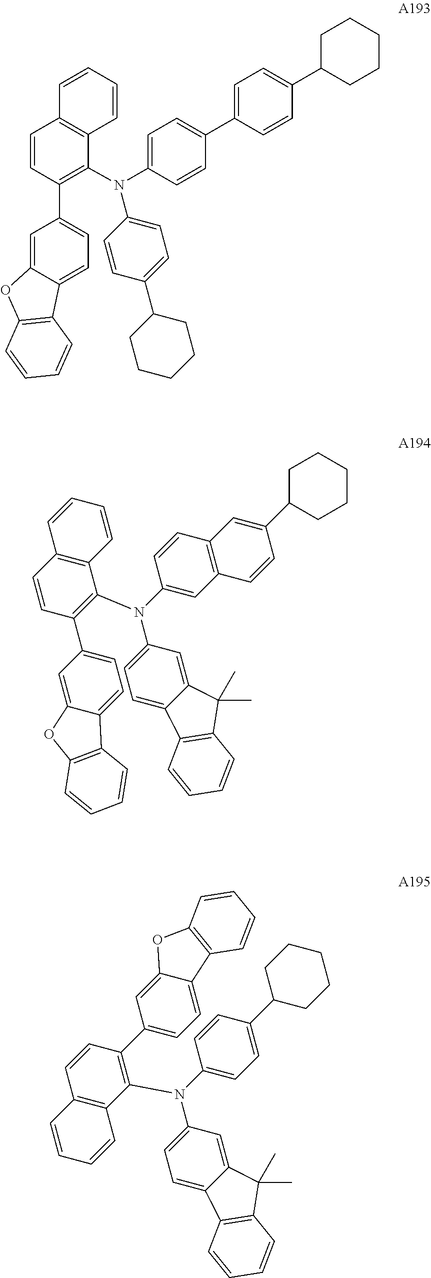 [SMILES: c1ccc2c(N(c3ccc(-c4ccc(C5CCCCC5)cc4)cc3)c3ccc(C4CCCCC4)cc3)c(-c3ccc4c(c3)oc3ccccc34)ccc2c1 ; CC1(C)c2ccccc2-c2ccc(N(c3ccc4cc(C5CCCCC5)ccc4c3)c3c(-c4ccc5c(c4)oc4ccccc45)ccc4ccccc34)cc21 ; CC1(C)c2ccccc2-c2ccc(N(c3ccc(C4CCCCC4)cc3)c3c(-c4ccc5c(c4)oc4ccccc45)ccc4ccccc34)cc21]

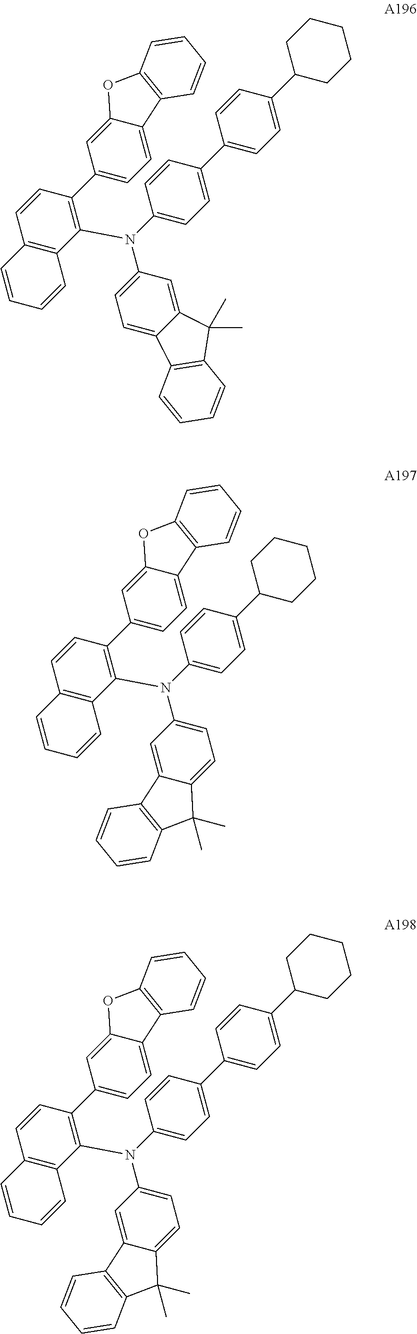 [SMILES: CC1(C)c2ccccc2-c2ccc(N(c3ccc(-c4ccc(C5CCCCC5)cc4)cc3)c3c(-c4ccc5c(c4)oc4ccccc45)ccc4ccccc34)cc21 ; CC1(C)c2ccccc2-c2cc(N(c3ccc(C4CCCCC4)cc3)c3c(-c4ccc5c(c4)oc4ccccc45)ccc4ccccc34)ccc21 ; CC1(C)c2ccccc2-c2cc(N(c3ccc(-c4ccc(C5CCCCC5)cc4)cc3)c3c(-c4ccc5c(c4)oc4ccccc45)ccc4ccccc34)ccc21]

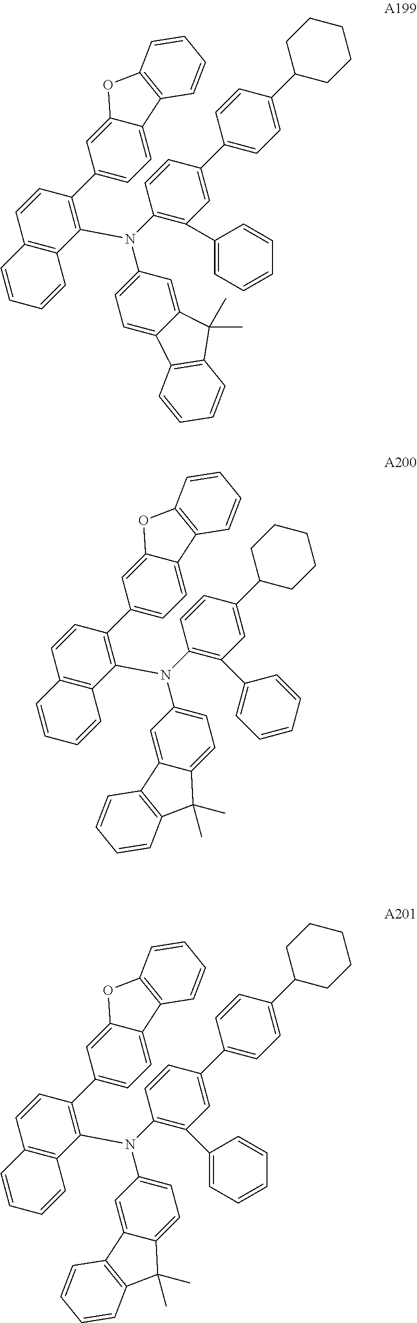 [SMILES: CC1(C)c2ccccc2-c2ccc(N(c3ccc(-c4ccc(C5CCCCC5)cc4)cc3-c3ccccc3)c3c(-c4ccc5c(c4)oc4ccccc45)ccc4ccccc34)cc21 ; CC1(C)c2ccccc2-c2cc(N(c3ccc(C4CCCCC4)cc3-c3ccccc3)c3c(-c4ccc5c(c4)oc4ccccc45)ccc4ccccc34)ccc21 ; CC1(C)c2ccccc2-c2cc(N(c3ccc(-c4ccc(C5CCCCC5)cc4)cc3-c3ccccc3)c3c(-c4ccc5c(c4)oc4ccccc45)ccc4ccccc34)ccc21]

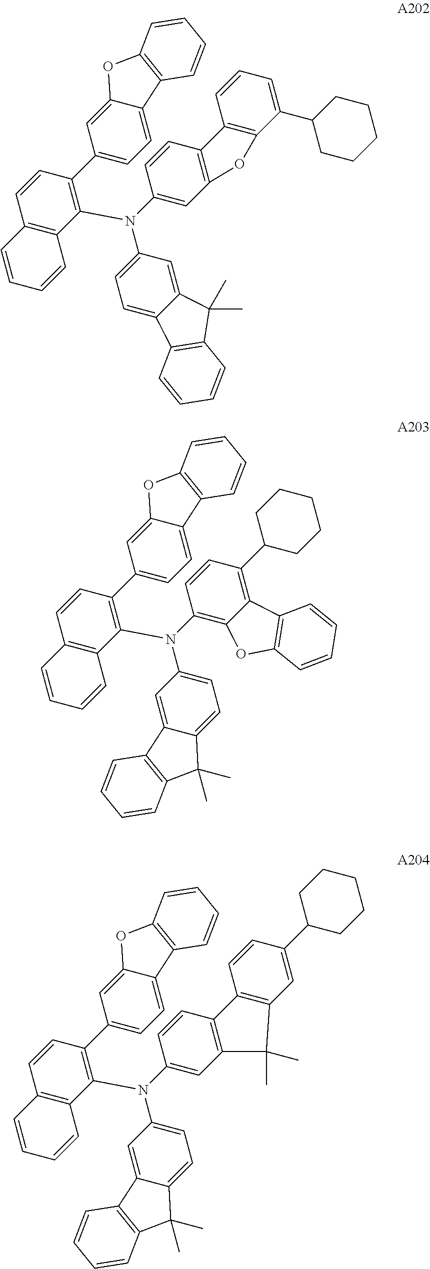

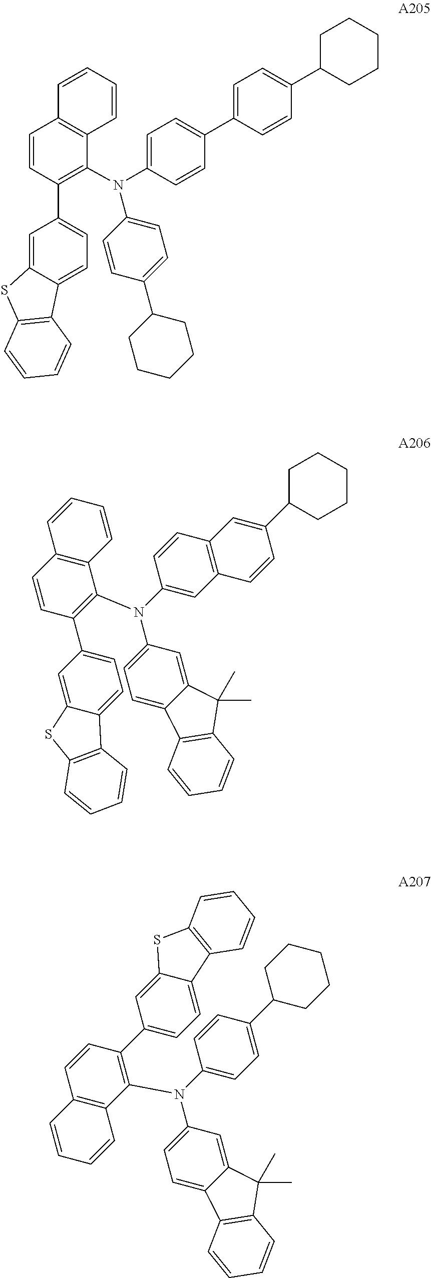 [SMILES: c1ccc2c(N(c3ccc(-c4ccc(C5CCCCC5)cc4)cc3)c3ccc(C4CCCCC4)cc3)c(-c3ccc4c(c3)sc3ccccc34)ccc2c1 ; CC1(C)c2ccccc2-c2ccc(N(c3ccc4cc(C5CCCCC5)ccc4c3)c3c(-c4ccc5c(c4)sc4ccccc45)ccc4ccccc34)cc21 ; CC1(C)c2ccccc2-c2ccc(N(c3ccc(C4CCCCC4)cc3)c3c(-c4ccc5c(c4)sc4ccccc45)ccc4ccccc34)cc21]

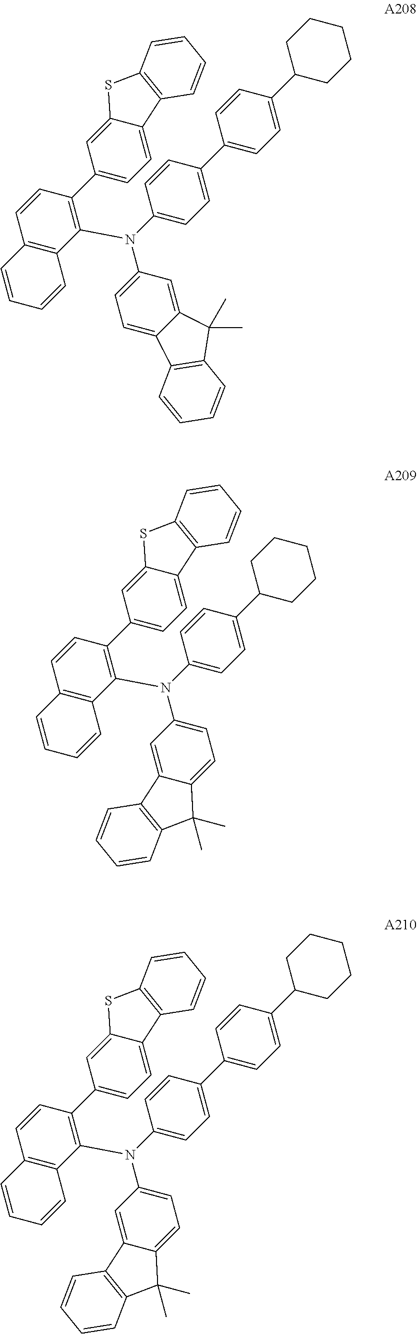 [SMILES: CC1(C)c2ccccc2-c2ccc(N(c3ccc(-c4ccc(C5CCCCC5)cc4)cc3)c3c(-c4ccc5c(c4)sc4ccccc45)ccc4ccccc34)cc21 ; CC1(C)c2ccccc2-c2cc(N(c3ccc(C4CCCCC4)cc3)c3c(-c4ccc5c(c4)sc4ccccc45)ccc4ccccc34)ccc21 ; CC1(C)c2ccccc2-c2cc(N(c3ccc(-c4ccc(C5CCCCC5)cc4)cc3)c3c(-c4ccc5c(c4)sc4ccccc45)ccc4ccccc34)ccc21]

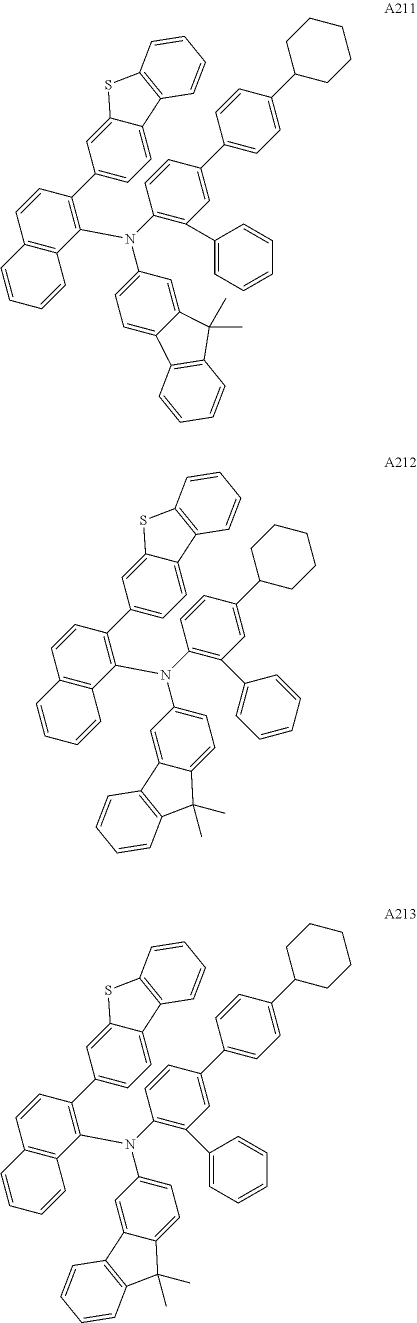 [SMILES: CC1(C)c2ccccc2-c2ccc(N(c3ccc(-c4ccc(C5CCCCC5)cc4)cc3-c3ccccc3)c3c(-c4ccc5c(c4)sc4ccccc45)ccc4ccccc34)cc21 ; CC1(C)c2ccccc2-c2cc(N(c3ccc(C4CCCCC4)cc3-c3ccccc3)c3c(-c4ccc5c(c4)sc4ccccc45)ccc4ccccc34)ccc21 ; CC1(C)c2ccccc2-c2cc(N(c3ccc(-c4ccc(C5CCCCC5)cc4)cc3-c3ccccc3)c3c(-c4ccc5c(c4)sc4ccccc45)ccc4ccccc34)ccc21]

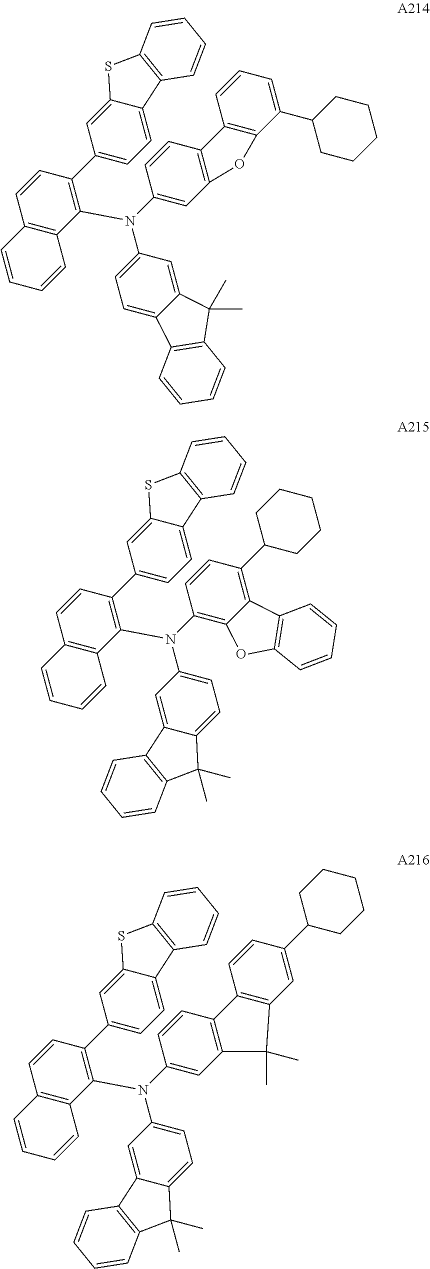

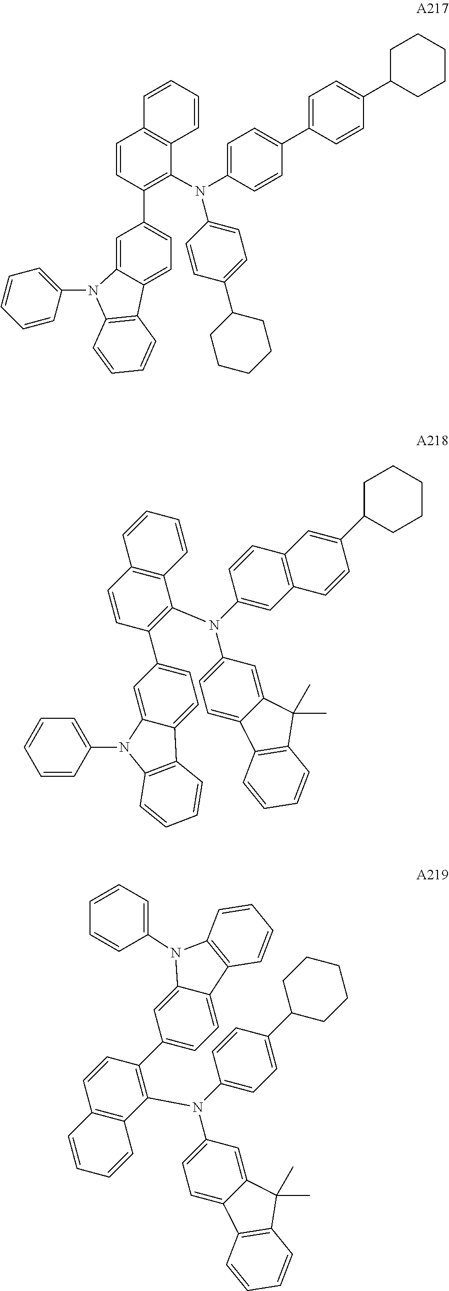 [SMILES: c1ccc(-n2c3ccccc3c3ccc(-c4ccc5ccccc5c4N(c4ccc(-c5ccc(C6CCCCC6)cc5)cc4)c4ccc(C5CCCCC5)cc4)cc32)cc1 ; CC1(C)c2ccccc2-c2ccc(N(c3ccc4cc(C5CCCCC5)ccc4c3)c3c(-c4ccc5c6ccccc6n(-c6ccccc6)c5c4)ccc4ccccc34)cc21 ; CC1(C)c2ccccc2-c2ccc(N(c3ccc(C4CCCCC4)cc3)c3c(-c4ccc5c6ccccc6n(-c6ccccc6)c5c4)ccc4ccccc34)cc21]

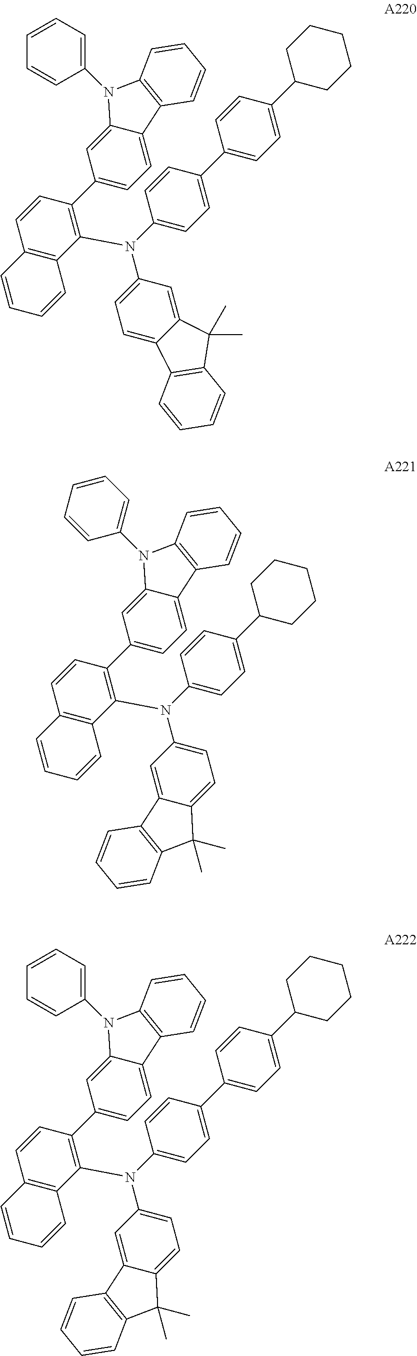 [SMILES: CC1(C)c2ccccc2-c2ccc(N(c3ccc(-c4ccc(C5CCCCC5)cc4)cc3)c3c(-c4ccc5c6ccccc6n(-c6ccccc6)c5c4)ccc4ccccc34)cc21 ; CC1(C)c2ccccc2-c2cc(N(c3ccc(C4CCCCC4)cc3)c3c(-c4ccc5c6ccccc6n(-c6ccccc6)c5c4)ccc4ccccc34)ccc21 ; CC1(C)c2ccccc2-c2cc(N(c3ccc(-c4ccc(C5CCCCC5)cc4)cc3)c3c(-c4ccc5c6ccccc6n(-c6ccccc6)c5c4)ccc4ccccc34)ccc21]

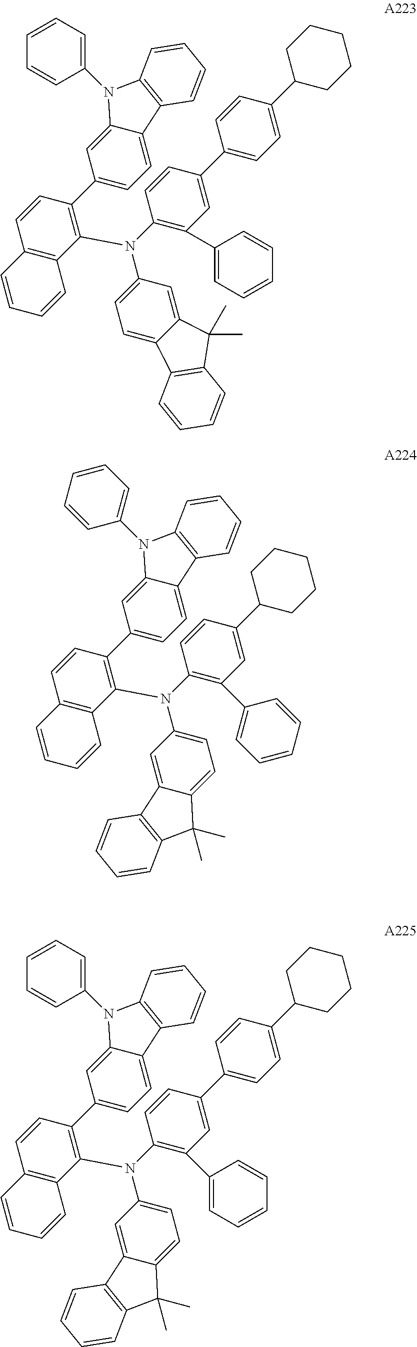 [SMILES: CC1(C)c2ccccc2-c2ccc(N(c3ccc(-c4ccc(C5CCCCC5)cc4)cc3-c3ccccc3)c3c(-c4ccc5c6ccccc6n(-c6ccccc6)c5c4)ccc4ccccc34)cc21 ; CC1(C)c2ccccc2-c2cc(N(c3ccc(C4CCCCC4)cc3-c3ccccc3)c3c(-c4ccc5c6ccccc6n(-c6ccccc6)c5c4)ccc4ccccc34)ccc21 ; CC1(C)c2ccccc2-c2cc(N(c3ccc(-c4ccc(C5CCCCC5)cc4)cc3-c3ccccc3)c3c(-c4ccc5c6ccccc6n(-c6ccccc6)c5c4)ccc4ccccc34)ccc21]

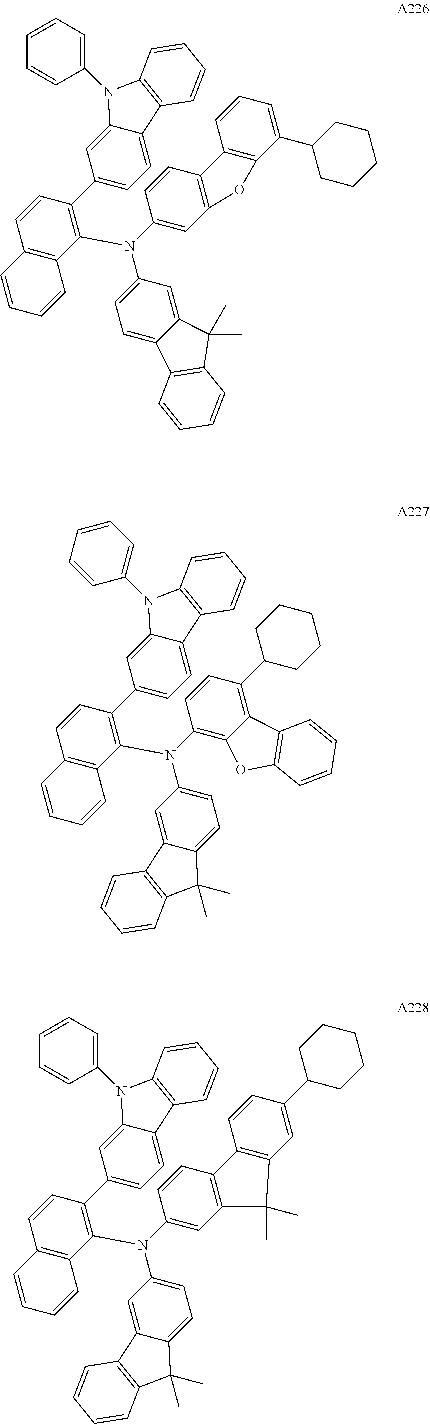 [SMILES: CC1(C)c2ccccc2-c2ccc(N(c3ccc4c(c3)oc3c(C5CCCCC5)cccc34)c3c(-c4ccc5c6ccccc6n(-c6ccccc6)c5c4)ccc4ccccc34)cc21 ; CC1(C)c2ccccc2-c2cc(N(c3c(-c4ccc5c6ccccc6n(-c6ccccc6)c5c4)ccc4ccccc34)c3ccc(C4CCCCC4)c4c3oc3ccccc34)ccc21 ; CC1(C)c2ccccc2-c2cc(N(c3ccc4c(c3)C(C)(C)c3cc(C5CCCCC5)ccc3-4)c3c(-c4ccc5c6ccccc6n(-c6ccccc6)c5c4)ccc4ccccc34)ccc21]

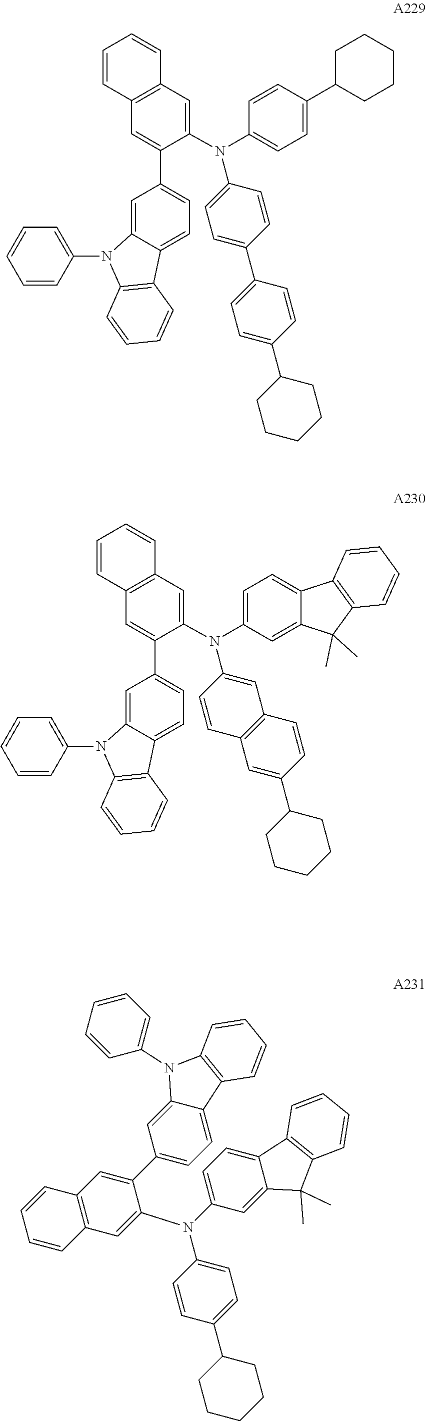 [SMILES: c1ccc(-n2c3ccccc3c3ccc(-c4cc5ccccc5cc4N(c4ccc(-c5ccc(C6CCCCC6)cc5)cc4)c4ccc(C5CCCCC5)cc4)cc32)cc1 ; CC1(C)c2ccccc2-c2ccc(N(c3ccc4cc(C5CCCCC5)ccc4c3)c3cc4ccccc4cc3-c3ccc4c5ccccc5n(-c5ccccc5)c4c3)cc21 ; CC1(C)c2ccccc2-c2ccc(N(c3ccc(C4CCCCC4)cc3)c3cc4ccccc4cc3-c3ccc4c5ccccc5n(-c5ccccc5)c4c3)cc21]

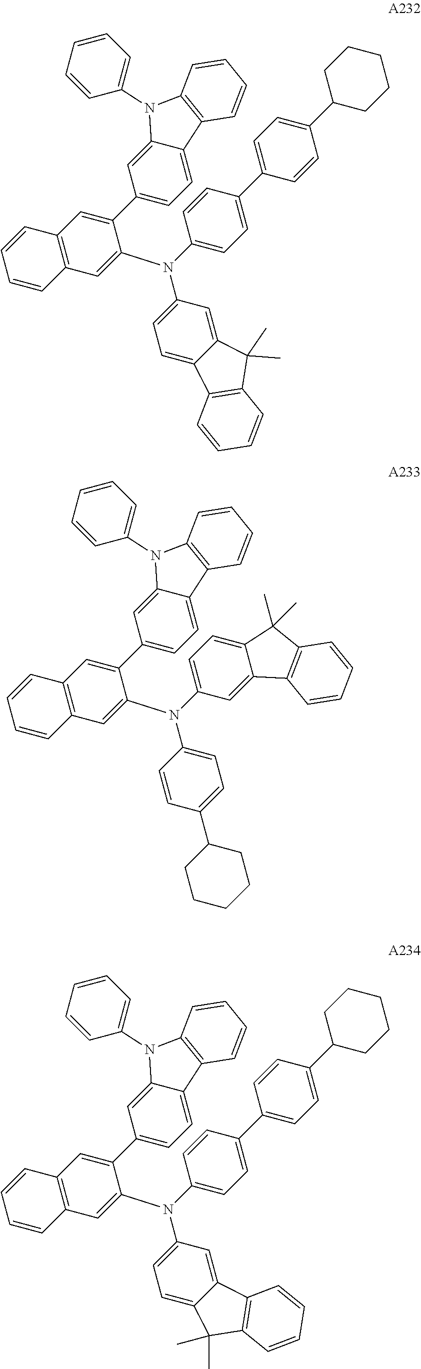 [SMILES: CC1(C)c2ccccc2-c2ccc(N(c3ccc(-c4ccc(C5CCCCC5)cc4)cc3)c3cc4ccccc4cc3-c3ccc4c5ccccc5n(-c5ccccc5)c4c3)cc21 ; CC1(C)c2ccccc2-c2cc(N(c3ccc(C4CCCCC4)cc3)c3cc4ccccc4cc3-c3ccc4c5ccccc5n(-c5ccccc5)c4c3)ccc21 ; CC1(C)c2ccccc2-c2cc(N(c3ccc(-c4ccc(C5CCCCC5)cc4)cc3)c3cc4ccccc4cc3-c3ccc4c5ccccc5n(-c5ccccc5)c4c3)ccc21]

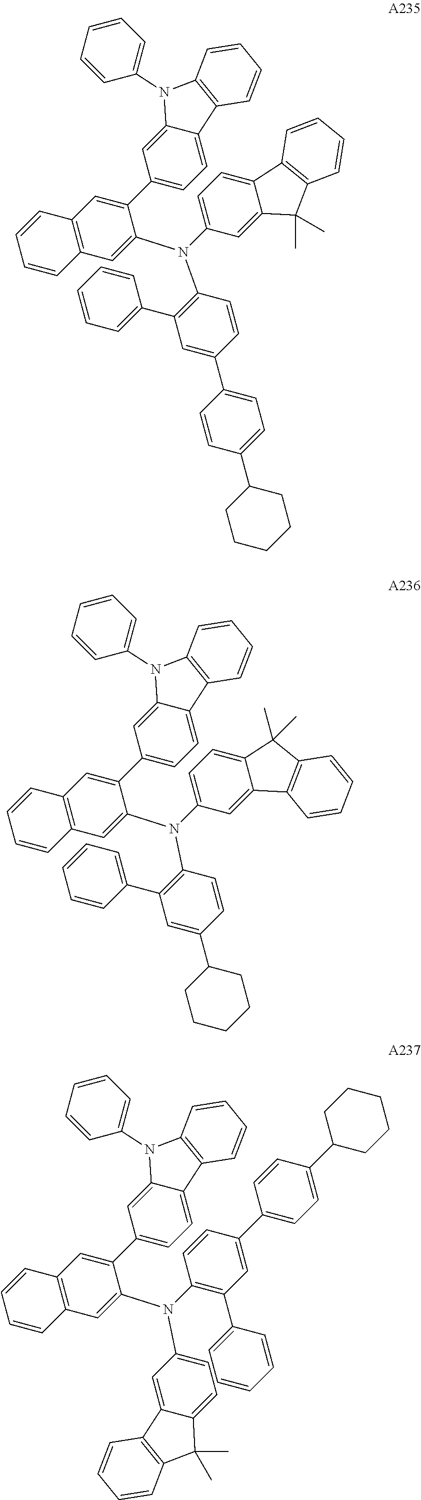 [SMILES: CC1(C)c2ccccc2-c2ccc(N(c3ccc(-c4ccc(C5CCCCC5)cc4)cc3-c3ccccc3)c3cc4ccccc4cc3-c3ccc4c5ccccc5n(-c5ccccc5)c4c3)cc21 ; CC1(C)c2ccccc2-c2cc(N(c3ccc(C4CCCCC4)cc3-c3ccccc3)c3cc4ccccc4cc3-c3ccc4c5ccccc5n(-c5ccccc5)c4c3)ccc21 ; CC1(C)c2ccccc2-c2cc(N(c3ccc(-c4ccc(C5CCCCC5)cc4)cc3-c3ccccc3)c3cc4ccccc4cc3-c3ccc4c5ccccc5n(-c5ccccc5)c4c3)ccc21]

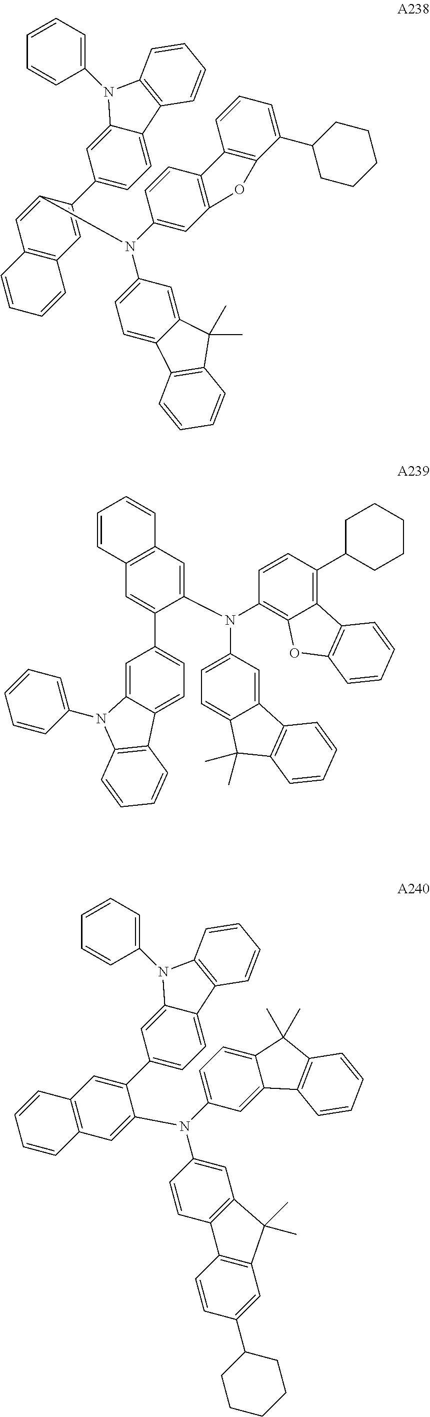 [SMILES: CC1(C)c2ccccc2-c2ccc(N(c3ccc4c(c3)oc3c(C5CCCCC5)cccc34)c3cc4ccccc4cc3-c3ccc4c5ccccc5n(-c5ccccc5)c4c3)cc21 ; CC1(C)c2ccccc2-c2cc(N(c3cc4ccccc4cc3-c3ccc4c5ccccc5n(-c5ccccc5)c4c3)c3ccc(C4CCCCC4)c4c3oc3ccccc34)ccc21 ; CC1(C)c2ccccc2-c2cc(N(c3ccc4c(c3)C(C)(C)c3cc(C5CCCCC5)ccc3-4)c3cc4ccccc4cc3-c3ccc4c5ccccc5n(-c5ccccc5)c4c3)ccc21]

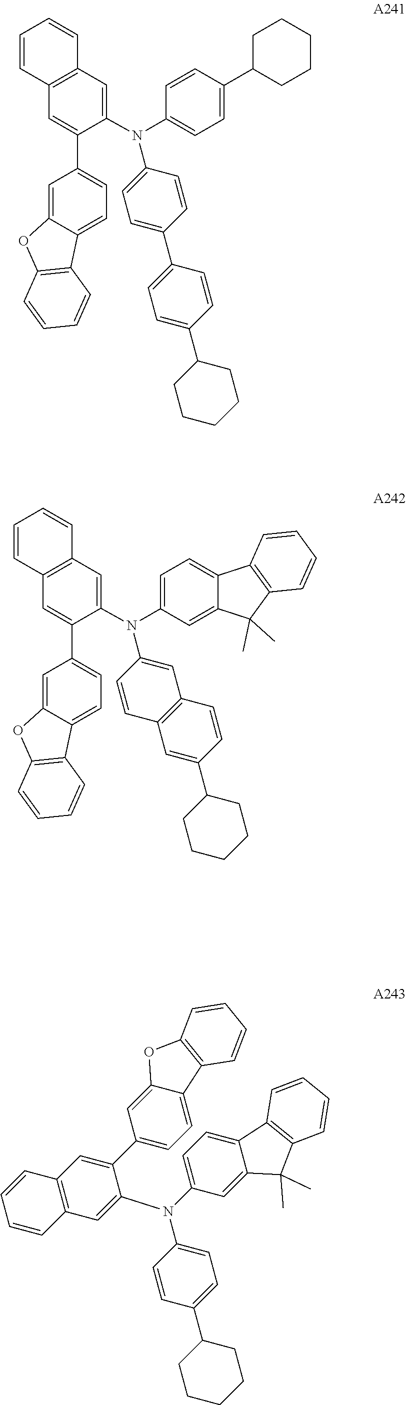 [SMILES: c1ccc2cc(N(c3ccc(-c4ccc(C5CCCCC5)cc4)cc3)c3ccc(C4CCCCC4)cc3)c(-c3ccc4c(c3)oc3ccccc34)cc2c1 ; CC1(C)c2ccccc2-c2ccc(N(c3ccc4cc(C5CCCCC5)ccc4c3)c3cc4ccccc4cc3-c3ccc4c(c3)oc3ccccc34)cc21 ; CC1(C)c2ccccc2-c2ccc(N(c3ccc(C4CCCCC4)cc3)c3cc4ccccc4cc3-c3ccc4c(c3)oc3ccccc34)cc21]

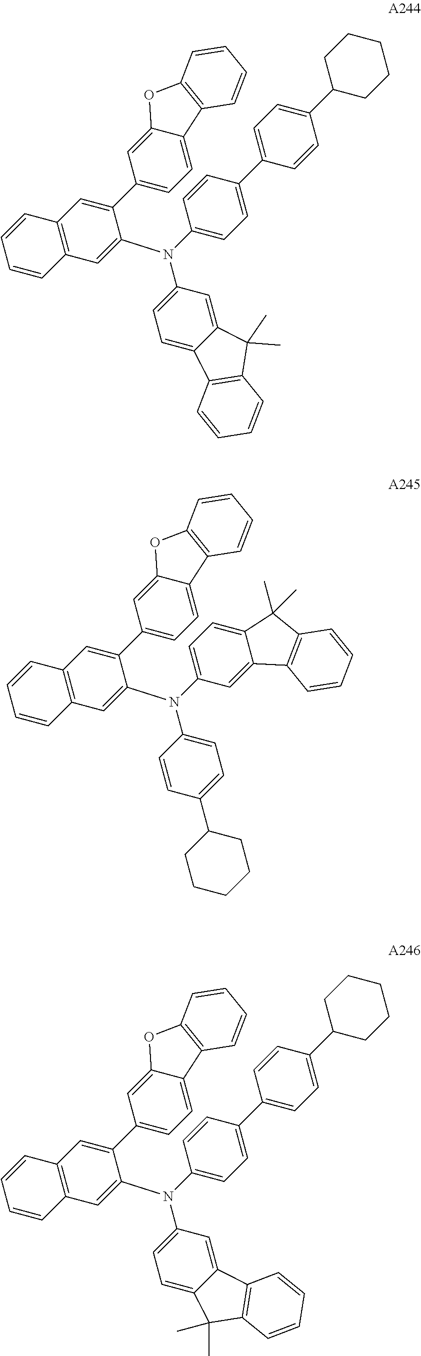 [SMILES: CC1(C)c2ccccc2-c2ccc(N(c3ccc(-c4ccc(C5CCCCC5)cc4)cc3)c3cc4ccccc4cc3-c3ccc4c(c3)oc3ccccc34)cc21 ; CC1(C)c2ccccc2-c2cc(N(c3ccc(C4CCCCC4)cc3)c3cc4ccccc4cc3-c3ccc4c(c3)oc3ccccc34)ccc21 ; CC1(C)c2ccccc2-c2cc(N(c3ccc(-c4ccc(C5CCCCC5)cc4)cc3)c3cc4ccccc4cc3-c3ccc4c(c3)oc3ccccc34)ccc21]

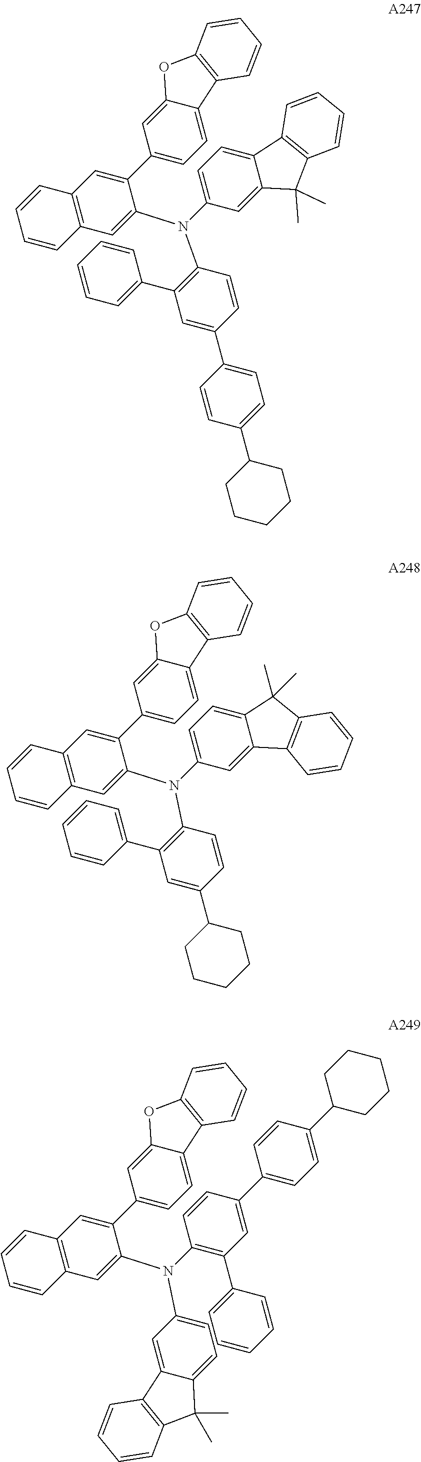 [SMILES: CC1(C)c2ccccc2-c2ccc(N(c3ccc(-c4ccc(C5CCCCC5)cc4)cc3-c3ccccc3)c3cc4ccccc4cc3-c3ccc4c(c3)oc3ccccc34)cc21 ; CC1(C)c2ccccc2-c2cc(N(c3ccc(C4CCCCC4)cc3-c3ccccc3)c3cc4ccccc4cc3-c3ccc4c(c3)oc3ccccc34)ccc21 ; CC1(C)c2ccccc2-c2cc(N(c3ccc(-c4ccc(C5CCCCC5)cc4)cc3-c3ccccc3)c3cc4ccccc4cc3-c3ccc4c(c3)oc3ccccc34)ccc21]

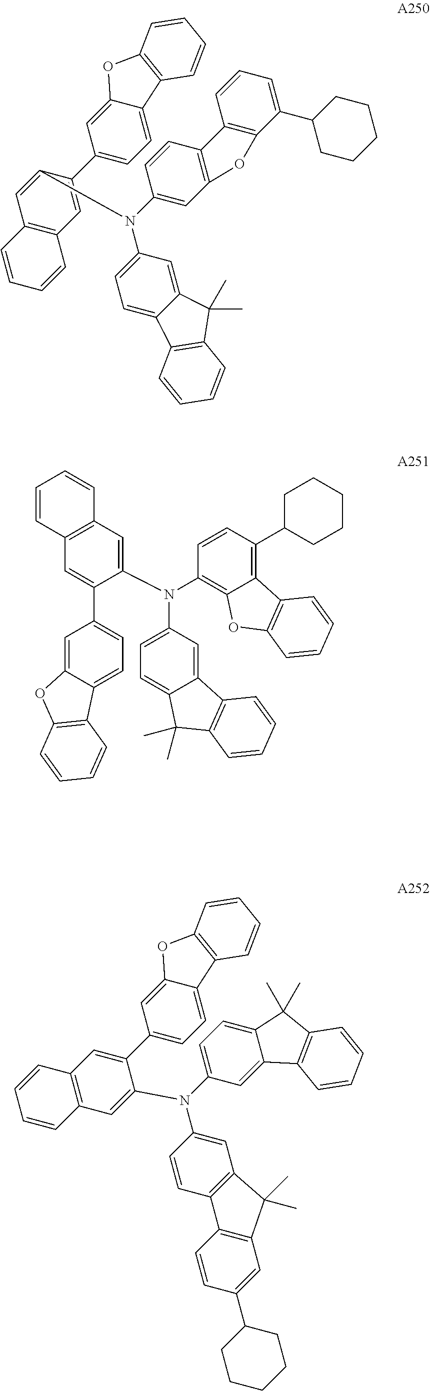 [SMILES: CC1(C)c2ccccc2-c2ccc(N(c3ccc4c(c3)oc3c(C5CCCCC5)cccc34)c3cc4ccccc4cc3-c3ccc4c(c3)oc3ccccc34)cc21 ; CC1(C)c2ccccc2-c2cc(N(c3cc4ccccc4cc3-c3ccc4c(c3)oc3ccccc34)c3ccc(C4CCCCC4)c4c3oc3ccccc34)ccc21 ; CC1(C)c2ccccc2-c2cc(N(c3ccc4c(c3)C(C)(C)c3cc(C5CCCCC5)ccc3-4)c3cc4ccccc4cc3-c3ccc4c(c3)oc3ccccc34)ccc21]

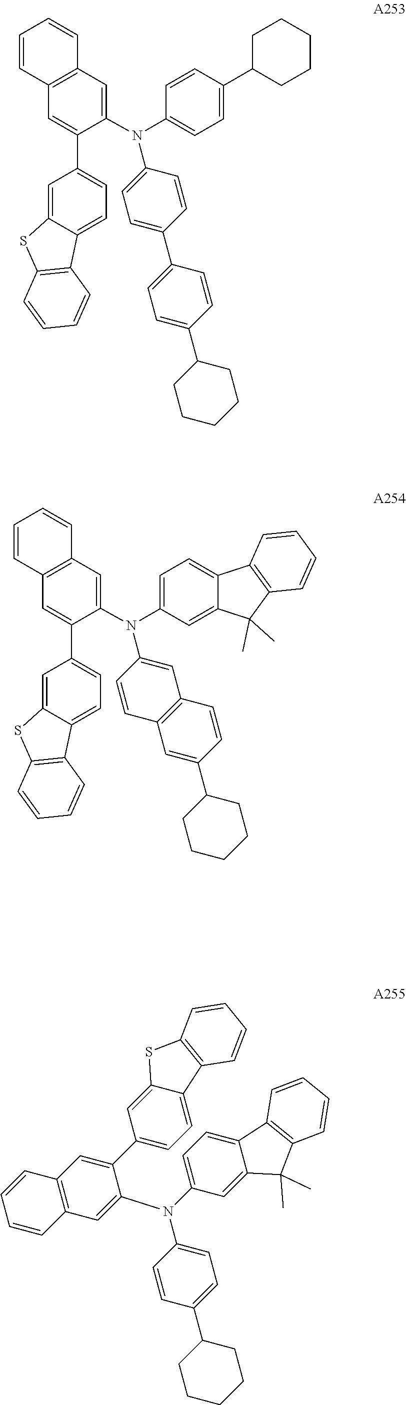 [SMILES: c1ccc2cc(N(c3ccc(-c4ccc(C5CCCCC5)cc4)cc3)c3ccc(C4CCCCC4)cc3)c(-c3ccc4c(c3)sc3ccccc34)cc2c1 ; CC1(C)c2ccccc2-c2ccc(N(c3ccc4cc(C5CCCCC5)ccc4c3)c3cc4ccccc4cc3-c3ccc4c(c3)sc3ccccc34)cc21 ; CC1(C)c2ccccc2-c2ccc(N(c3ccc(C4CCCCC4)cc3)c3cc4ccccc4cc3-c3ccc4c(c3)sc3ccccc34)cc21]

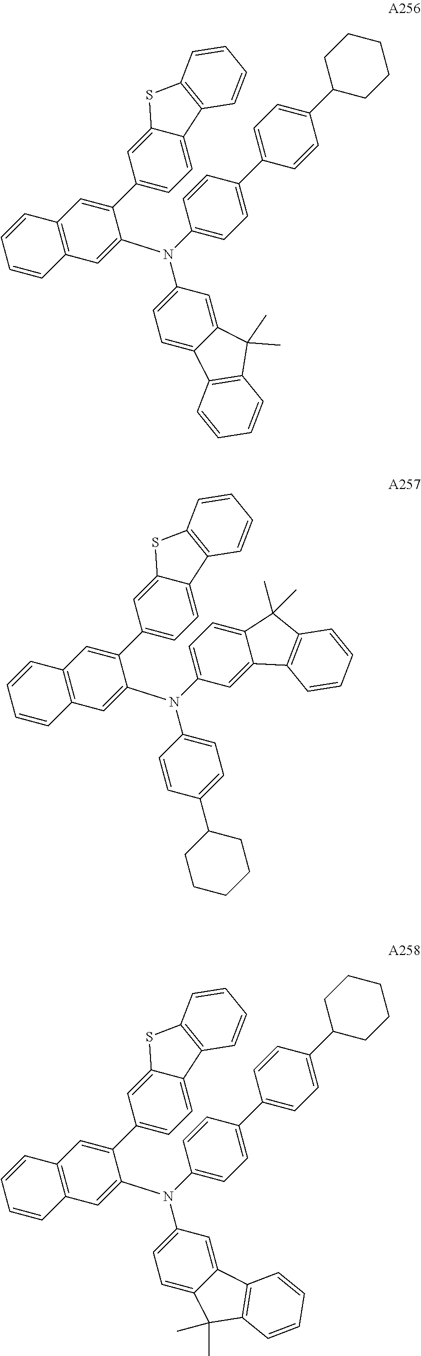 [SMILES: CC1(C)c2ccccc2-c2ccc(N(c3ccc(-c4ccc(C5CCCCC5)cc4)cc3)c3cc4ccccc4cc3-c3ccc4c(c3)sc3ccccc34)cc21 ; CC1(C)c2ccccc2-c2cc(N(c3ccc(C4CCCCC4)cc3)c3cc4ccccc4cc3-c3ccc4c(c3)sc3ccccc34)ccc21 ; CC1(C)c2ccccc2-c2cc(N(c3ccc(-c4ccc(C5CCCCC5)cc4)cc3)c3cc4ccccc4cc3-c3ccc4c(c3)sc3ccccc34)ccc21]

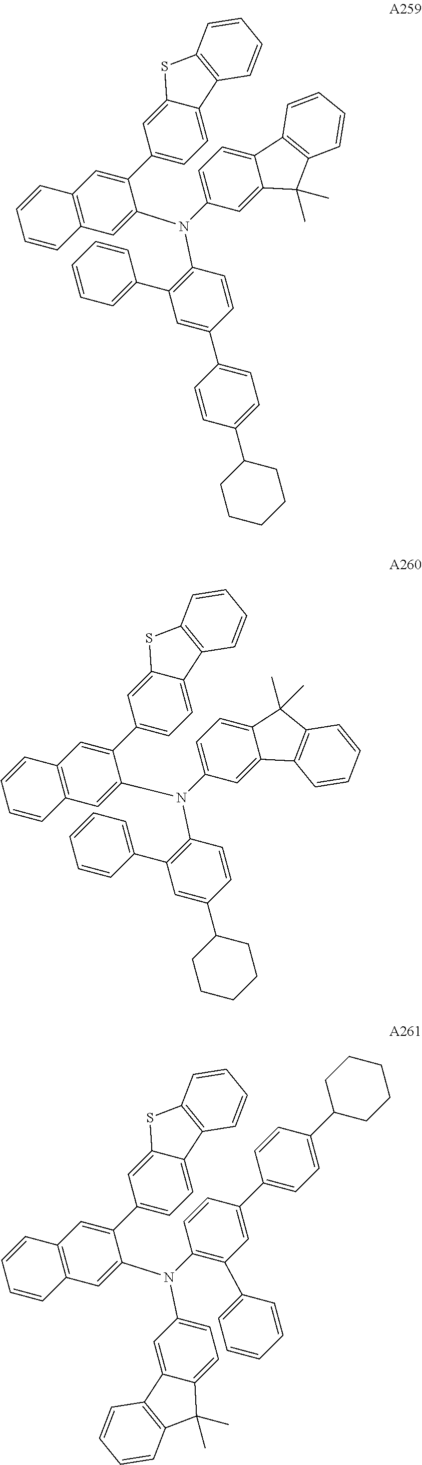 [SMILES: CC1(C)c2ccccc2-c2ccc(N(c3ccc(-c4ccc(C5CCCCC5)cc4)cc3-c3ccccc3)c3cc4ccccc4cc3-c3ccc4c(c3)sc3ccccc34)cc21 ; CC1(C)c2ccccc2-c2cc(N(c3ccc(C4CCCCC4)cc3-c3ccccc3)c3cc4ccccc4cc3-c3ccc4c(c3)sc3ccccc34)ccc21 ; CC1(C)c2ccccc2-c2cc(N(c3ccc(-c4ccc(C5CCCCC5)cc4)cc3-c3ccccc3)c3cc4ccccc4cc3-c3ccc4c(c3)sc3ccccc34)ccc21]

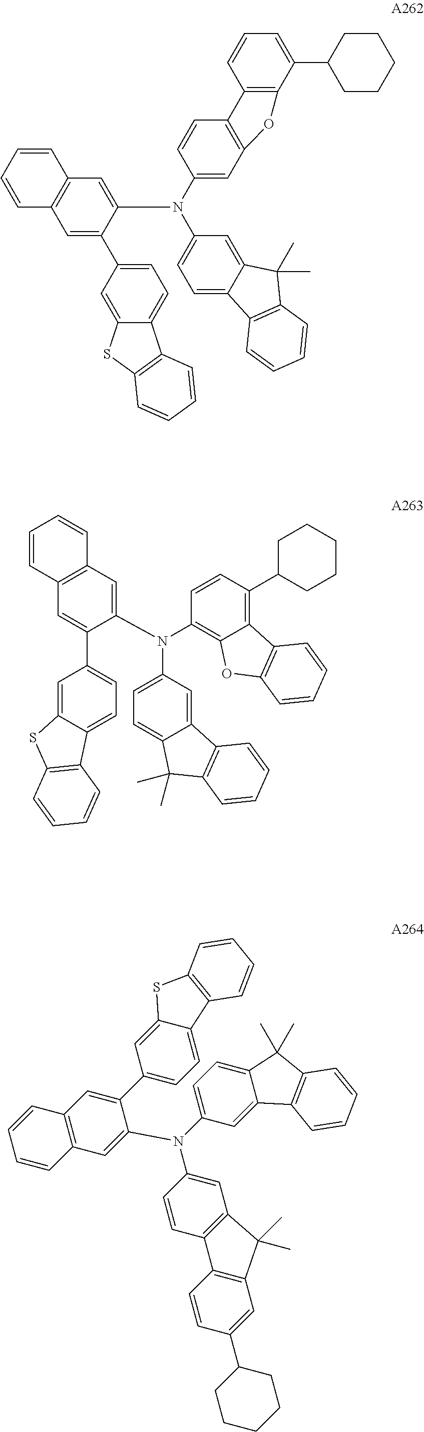 [SMILES: CC1(C)c2ccccc2-c2ccc(N(c3ccc4c(c3)oc3c(C5CCCCC5)cccc34)c3cc4ccccc4cc3-c3ccc4c(c3)sc3ccccc34)cc21 ; CC1(C)c2ccccc2-c2cc(N(c3cc4ccccc4cc3-c3ccc4c(c3)sc3ccccc34)c3ccc(C4CCCCC4)c4c3oc3ccccc34)ccc21 ; CC1(C)c2ccccc2-c2cc(N(c3ccc4c(c3)C(C)(C)c3cc(C5CCCCC5)ccc3-4)c3cc4ccccc4cc3-c3ccc4c(c3)sc3ccccc34)ccc21]

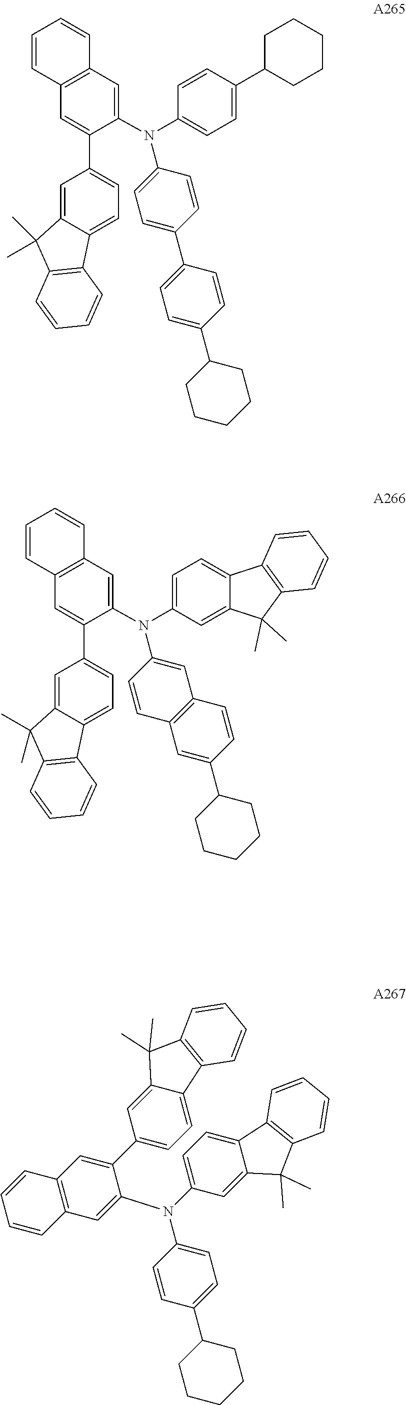 [SMILES: CC1(C)c2ccccc2-c2ccc(-c3cc4ccccc4cc3N(c3ccc(-c4ccc(C5CCCCC5)cc4)cc3)c3ccc(C4CCCCC4)cc3)cc21 ; CC1(C)c2ccccc2-c2ccc(-c3cc4ccccc4cc3N(c3ccc4c(c3)C(C)(C)c3ccccc3-4)c3ccc4cc(C5CCCCC5)ccc4c3)cc21 ; CC1(C)c2ccccc2-c2ccc(-c3cc4ccccc4cc3N(c3ccc(C4CCCCC4)cc3)c3ccc4c(c3)C(C)(C)c3ccccc3-4)cc21]

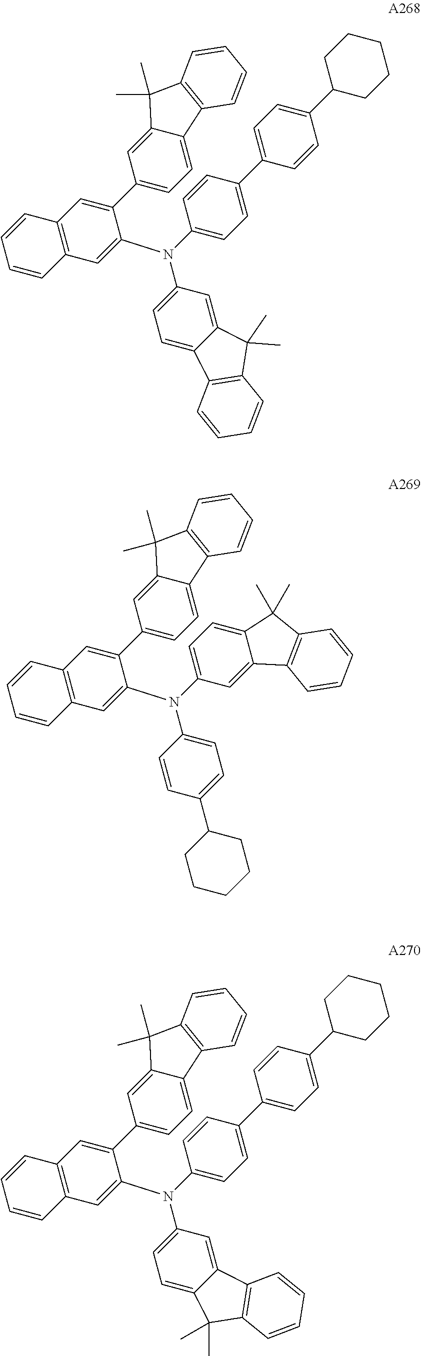 [SMILES: CC1(C)c2ccccc2-c2ccc(-c3cc4ccccc4cc3N(c3ccc(-c4ccc(C5CCCCC5)cc4)cc3)c3ccc4c(c3)C(C)(C)c3ccccc3-4)cc21 ; CC1(C)c2ccccc2-c2cc(N(c3ccc(C4CCCCC4)cc3)c3cc4ccccc4cc3-c3ccc4c(c3)C(C)(C)c3ccccc3-4)ccc21 ; CC1(C)c2ccccc2-c2cc(N(c3ccc(-c4ccc(C5CCCCC5)cc4)cc3)c3cc4ccccc4cc3-c3ccc4c(c3)C(C)(C)c3ccccc3-4)ccc21]

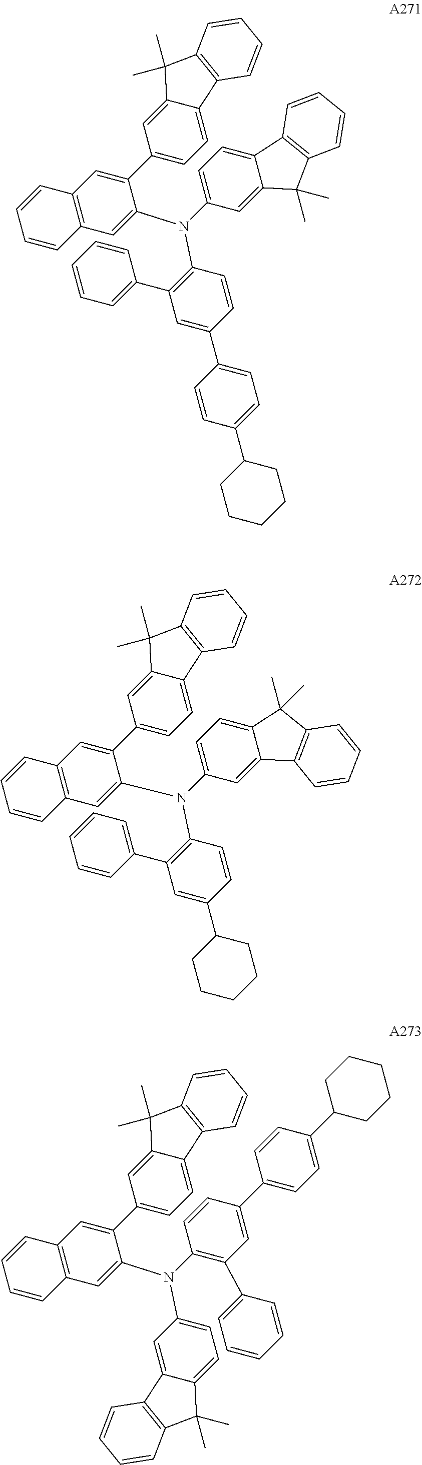 [SMILES: CC1(C)c2ccccc2-c2ccc(-c3cc4ccccc4cc3N(c3ccc4c(c3)C(C)(C)c3ccccc3-4)c3ccc(-c4ccc(C5CCCCC5)cc4)cc3-c3ccccc3)cc21 ; CC1(C)c2ccccc2-c2cc(N(c3ccc(C4CCCCC4)cc3-c3ccccc3)c3cc4ccccc4cc3-c3ccc4c(c3)C(C)(C)c3ccccc3-4)ccc21 ; CC1(C)c2ccccc2-c2cc(N(c3ccc(-c4ccc(C5CCCCC5)cc4)cc3-c3ccccc3)c3cc4ccccc4cc3-c3ccc4c(c3)C(C)(C)c3ccccc3-4)ccc21]

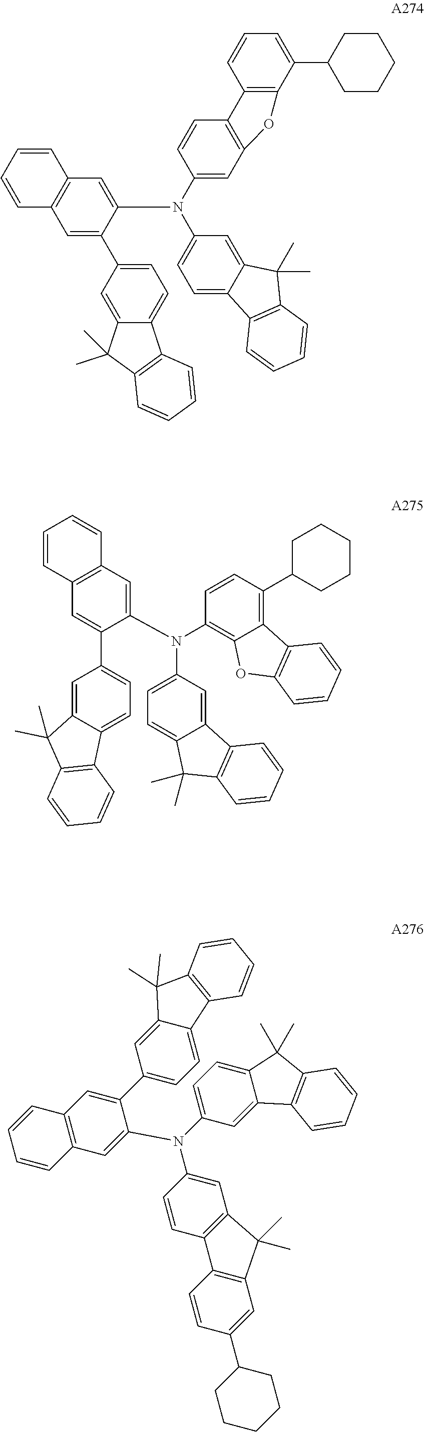 [SMILES: CC1(C)c2ccccc2-c2ccc(-c3cc4ccccc4cc3N(c3ccc4c(c3)C(C)(C)c3ccccc3-4)c3ccc4c(c3)oc3c(C5CCCCC5)cccc34)cc21 ; CC1(C)c2ccccc2-c2cc(N(c3cc4ccccc4cc3-c3ccc4c(c3)C(C)(C)c3ccccc3-4)c3ccc(C4CCCCC4)c4c3oc3ccccc34)ccc21 ; CC1(C)c2ccccc2-c2cc(N(c3ccc4c(c3)C(C)(C)c3cc(C5CCCCC5)ccc3-4)c3cc4ccccc4cc3-c3ccc4c(c3)C(C)(C)c3ccccc3-4)ccc21]

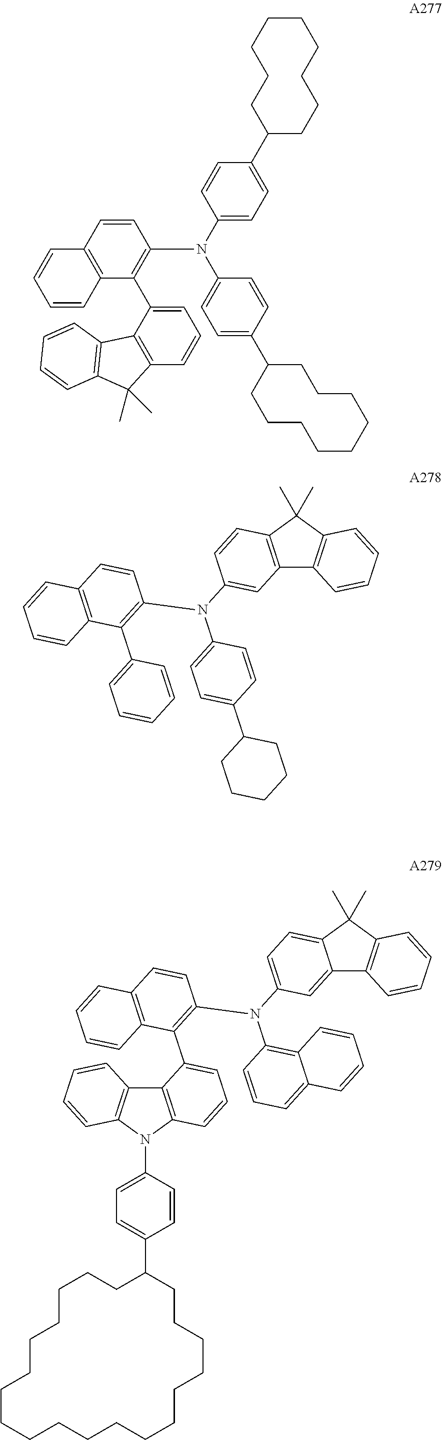

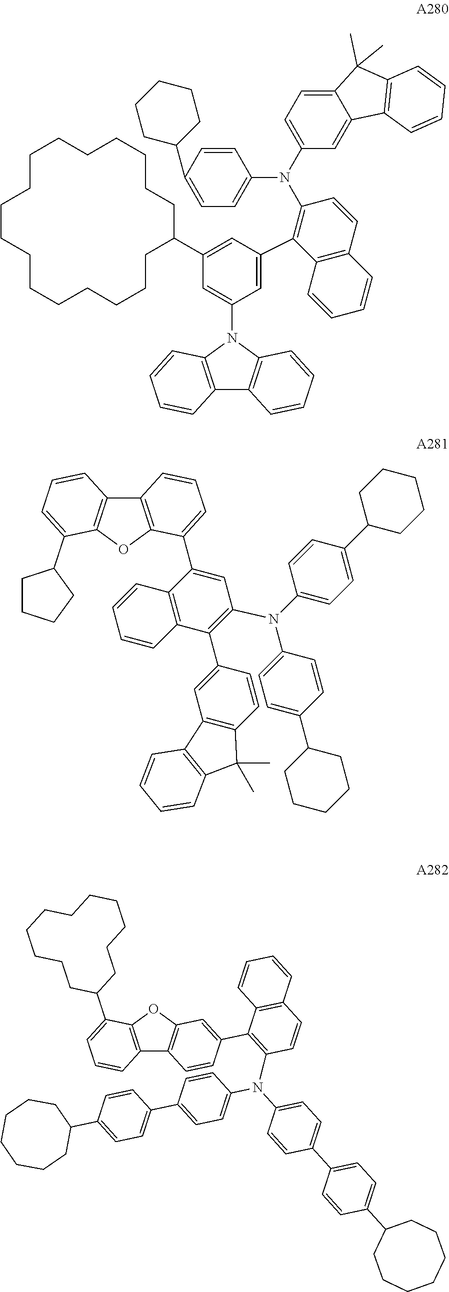 [SMILES: CC1(C)c2ccccc2-c2cc(N(c3ccc(C4CCCCC4)cc3)c3ccc4ccccc4c3-c3cc(C4CCCCCCCCCCCCCCCCC4)cc(-n4c5ccccc5c5ccccc54)c3)ccc21 ; CC1(C)c2ccccc2-c2cc(-c3c(N(c4ccc(C5CCCCC5)cc4)c4ccc(C5CCCCC5)cc4)cc(-c4cccc5c4oc4c(C6CCCC6)cccc45)c4ccccc34)ccc21 ; c1ccc2c(-c3ccc4c(c3)oc3c(C5CCCCCCCCCCC5)cccc34)c(N(c3ccc(-c4ccc(C5CCCCCCC5)cc4)cc3)c3ccc(-c4ccc(C5CCCCCCC5)cc4)cc3)ccc2c1]

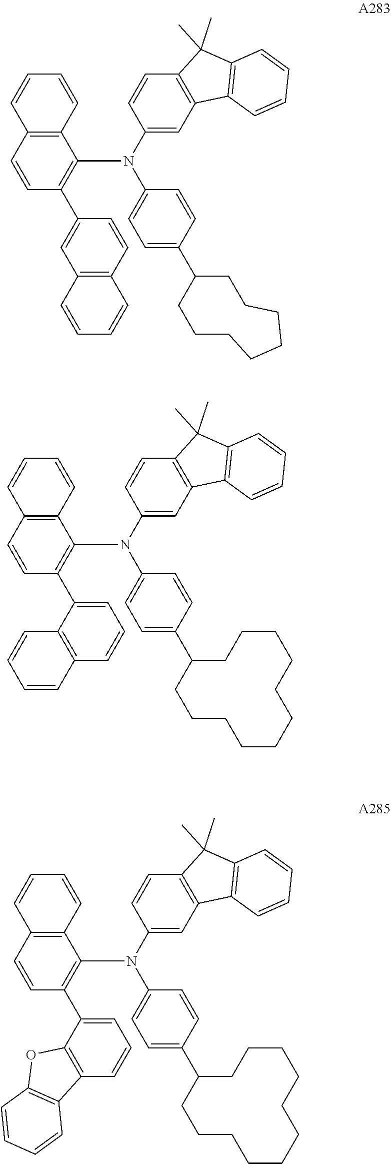 [SMILES: CC1(C)c2ccccc2-c2cc(N(c3ccc(C4CCCCCCCC4)cc3)c3c(-c4ccc5ccccc5c4)ccc4ccccc34)ccc21 ; CC1(C)c2ccccc2-c2cc(N(c3ccc(C4CCCCCCCCCCC4)cc3)c3c(-c4cccc5ccccc45)ccc4ccccc34)ccc21 ; CC1(C)c2ccccc2-c2cc(N(c3ccc(C4CCCCCCCCCCC4)cc3)c3c(-c4cccc5c4oc4ccccc45)ccc4ccccc34)ccc21]

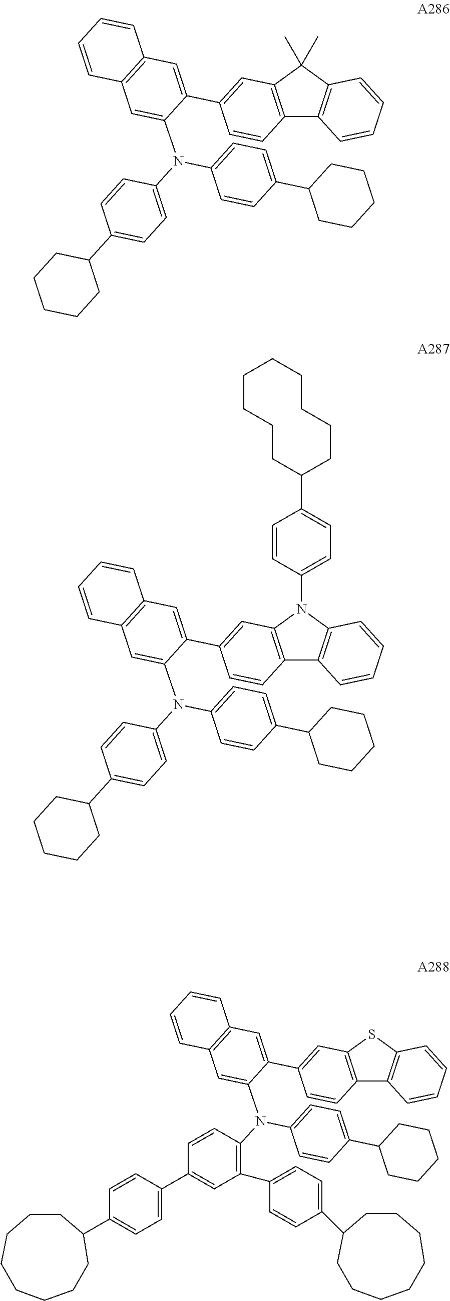 [SMILES: CC1(C)c2ccccc2-c2ccc(-c3cc4ccccc4cc3N(c3ccc(C4CCCCC4)cc3)c3ccc(C4CCCCC4)cc3)cc21 ; c1ccc2cc(N(c3ccc(C4CCCCC4)cc3)c3ccc(C4CCCCC4)cc3)c(-c3ccc4c5ccccc5n(-c5ccc(C6CCCCCCCCC6)cc5)c4c3)cc2c1 ; c1ccc2cc(N(c3ccc(C4CCCCC4)cc3)c3ccc(-c4ccc(C5CCCCCCCC5)cc4)cc3-c3ccc(C4CCCCCCCC4)cc3)c(-c3ccc4c(c3)sc3ccccc34)cc2c1]

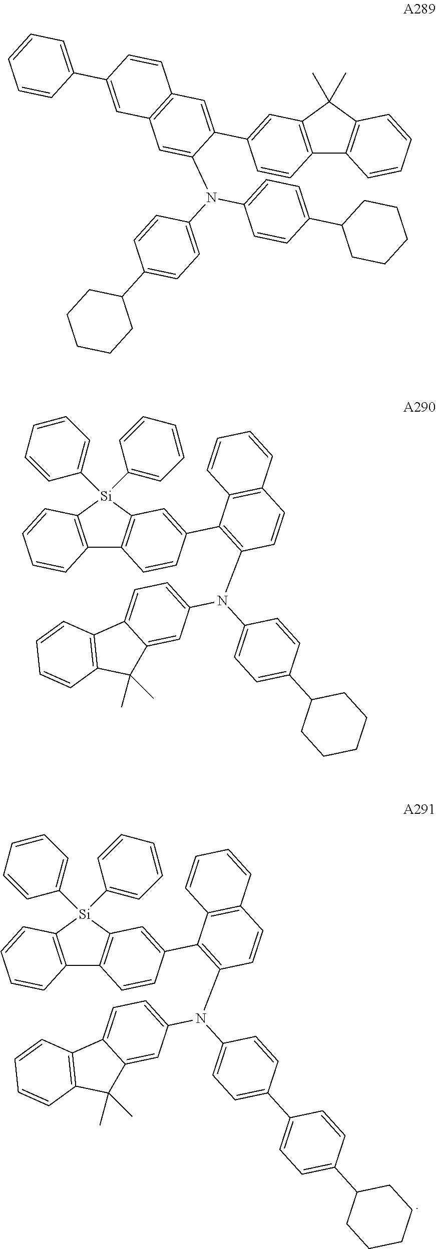 [SMILES: CC1(C)c2ccccc2-c2ccc(-c3cc4ccc(-c5ccccc5)cc4cc3N(c3ccc(C4CCCCC4)cc3)c3ccc(C4CCCCC4)cc3)cc21 ; CC1(C)c2ccccc2-c2ccc(N(c3ccc(C4CCCCC4)cc3)c3ccc4ccccc4c3-c3ccc4c(c3)[Si](c3ccccc3)(c3ccccc3)c3ccccc3-4)cc21 ; CC1(C)c2ccccc2-c2ccc(N(c3ccc(-c4ccc(C5CCCCC5)cc4)cc3)c3ccc4ccccc4c3-c3ccc4c(c3)[Si](c3ccccc3)(c3ccccc3)c3ccccc3-4)cc21]

26. The organic electroluminescent device according to claim 23, wherein, in the direction from the cathode layer to the anode layer, the organic layer comprises a light emitting layer and an electron blocking layer in sequence,
the light emitting layer contains the compound I, and the electron blocking layer contains the compound II; or
the light emitting layer contains the compound II, and the electron blocking layer contains the compound I.

27. The organic electroluminescent device according to claim 26, wherein, the light emitting layer contains a first host material, a second host material and a doping material;
the first host material is the compound I, the electron blocking layer contains the compound II, or, the first host material is the compound II, and the electron blocking layer contains the compound I;
preferably, the mass ratio of the first host material to the second host material is 0.01:1-1.5:1, preferably is 0-1:1-1:1.

28. (canceled)

29. The organic electroluminescent device according to claim 26, wherein, the thickness of the light emitting layer is 10-60 nm, preferably is 20-50 nm;
preferably, the thickness of the electron blocking layer is 2-100 nm, preferably is 3-90 nm.

30. (canceled)

31. The organic electroluminescent device according to claim 27, wherein, the organic layer further comprises a hole injection layer, a hole transporting layer, an electron transporting layer and an electron injection layer, and in the direction from the anode layer to the cathode layer, the organic layer comprises the hole injection layer, the hole transporting layer, the electron blocking layer, the light emitting layer, the electron transporting layer and the electron injection layer in sequence.

32. A display device, wherein, the display device comprises the organic electroluminescent device according to claim 1 or according to claim 25.
